(19) United States
(12) Patent Application Publication
(10) Pub. No.: US 2024/0150453 A1
(43) Pub. Date: May 9, 2024

(54) METHODS OF PREDICTING RESPONSE TO ANTI-TNF BLOCKADE IN INFLAMMATORY BOWEL DISEASE

(71) Applicants: Massachusetts Institute of Technology, Cambridge, MA (US); Seattle Children's Hospital DBA Seattle Children's Research Institute, Seattle, WA (US); The Children's Medical Center Corporation, Boston, MA (US)

(72) Inventors: Alexander K. Shalek, Cambridge, MA (US); Leslie Kean, Seattle, WA (US); Jose Ordovas-Montanes, Boston, MA (US); Hengqi Zheng, Seattle, WA (US); Benjamin Doran, Cambridge, MA (US); Kyle Kimler, Boston, MA (US)

(21) Appl. No.: 18/280,547

(22) PCT Filed: Mar. 9, 2022
(86) PCT No.: PCT/US2022/019582

§ 371 Date: Sep. 6, 2023

Related U.S. Application Data
(60) Provisional application No. 63/158,711, filed on Mar. 9, 2021.

(51) Int. Cl. C07K 16/24; A61P 1/00; A61P 29/00; C12Q 1/6883; G01N 33/50

(52) U.S. Cl. CPC C07K 16/241; A61P 1/00; A61P 29/00; C12Q 1/6883; G01N 33/5091; C12Q 2600/106; C12Q 2600/158; G01N 2800/065; G01N 2800/52

(57) ABSTRACT

The subject matter disclosed herein is generally directed to stratifying and treating inflammatory diseases. In particular, the present invention provides for detecting treatment naive cell states that predict the response of a subject having inflammatory bowel disease to anti-TNF-blockade. The cells are T cells, NK cells, innate lymphoid cells, myeloid and/or epithelial cell subsets defined by their gene expression as determined by single cell RNA sequencing. Alternatively, the responders are identified by gene expression in a sample.

### CROSS-REFERENCE TO RELATED APPLICATIONS

This application claims the benefit of U.S. Provisional Application No. 63/158,711, filed Mar. 9, 2021. The entire contents of the above-identified application are hereby fully incorporated herein by reference.

### STATEMENT REGARDING FEDERALLY SPONSORED RESEARCH

This invention was made with government support under Grant Nos. DK034854, AI118672, HL095791, HL158504, and AI051731 awarded by the National Institutes of Health. The government has certain rights in the invention.

### TECHNICAL FIELD

The subject matter disclosed herein is generally directed to determining whether a subject suffering from inflammatory bowel disease (IBD) will respond to anti-TNF-blockade and treating the subject.

### BACKGROUND

Inflammatory bowel diseases (IBDs) arise when homeostatic mechanisms regulating gastrointestinal (GI) tract tissue integrity, nutrient absorption, and protective immunity are replaced by pathogenic inflammation (Baumgart and Sandborn, 2012; Chang, 2020; Corridoni et al., 2020a; Friedrich et al., 2019; Graham and Xavier, 2020; Selin et al., 2021). The initiating triggers are not fully known, but host genetics and the microbiome are being increasingly appreciated to play important, and in some cases causal roles in the IBDs (Chang, 2020; Cohen et al., 2019; Franzosa et al., 2019; Jain et al., 2021; Limon et al., 2019). Though symptoms are widespread and overlapping, the endoscopic inflammation in the IBDs is often anatomically restricted: ulcerative colitis (UC) manifests primarily as an superficial inflammatory response restricted to the colon, while Crohn's disease (CD) presents predominantly in the terminal ileum and the proximal colon, though lesions may develop anywhere along the gastrointestinal tract (Baumgart and Sandborn, 2012; Chang, 2020; Kobayashi et al., 2020; Roda et al., 2020). Among the IBDs, pediatric-onset Crohn's disease (pediCD) is particularly common (25% of all IBD cases, 60-70% of pediatric IBD) and is a debilitating form due to its early presentation, impact on the terminal ileum and proximal colon, and the lack of disease-specific therapies developed with children in mind (Hyams et al., 1991; Ruemmele et al., 2014; Sykora et al., 2018; Turner et al., 2012; Ye et al., 2020). In contrast to pediCD, a group of pediatric disorders termed functional gastrointestinal disorders (FGIDs) include GI symptoms but lack laboratory markers, endoscopic findings, and histologic evidence associated with inflammation (Black et al., 2020; Hyams et al., 2016; McOmber and Shulman, 2008; Santucci et al., 2020). FGID thus represents a critical non-inflamed control cohort with which to contextualize the inflammation observed in pediCD.

The current standard of care for pediCD (as with adult CD) is tailored to the patient's disease location, clinical behavior and severity, though use of prednisone, immunomodulators, as well as biologics including anti-TNF-α monoclonal antibodies, are common (Hyams et al., 1991; Ruemmele et al., 2014; Turner et al., 2012). While targeting TNF is common across many autoimmune and inflammatory conditions, it is not successful in all patients, and many go on to develop anti-TNF-refractory disease. It is of tremendous importance for the field to precisely understand and characterize for which patients anti-TNF therapy is not necessary, in which patients it may succeed in controlling disease, and which patients will be refractory to treatment. Several ideas based on individual immunogenicity and pharmacokinetics have been proposed to explain TNF-refractory disease, including gender (M>F), low albumin levels, high BMI, and high baseline C-Reactive Protein (CRP) (Atreya et al., 2020; Digby-Bell et al., 2020). However, no single identifiable clinical or biochemical biomarker reliably predicts disease response versus resistance to anti-TNF antibodies suggesting a more complex etiology (Stevens et al., 2018). It would be of tremendous interest for the field to precisely understand in which patients anti-TNF therapy is not necessary (not on anti-TNF: NOA), may succeed in controlling disease (full-responders: FRs), and which patients will either immediately or progressively gain resistance to treatment (partial-responders: PRs).

The primary cellular lineages sampled from intestinal biopsies of CD patients (from the terminal ileum or colon) represent both the epithelium and lamina propria, and include epithelial cells, stromal cells, hematopoietic cells and neuronal processes whose cell bodies are present outside of these regions (Buisine et al., 2001; Leeb et al., 2003; Leonard et al., 1995; Lilja et al., 2000; Müller et al., 1998; Souza et al., 1999; Stappenbeck and McGovern, 2017; Takayama et al., 2010). Alterations to all cellular lineages have been implicated in CD (Furey et al., 2019; Martin et al., 2019). Histologically, CD is characterized by a granulomatous inflammation, and noted alterations in almost every leukocyte cell type studied including an increase of cytotoxic lymphocytes, potential but equivocal alterations in γδ T cells, increases in mast cells and their production of TNF, activation and shifts in antibody isotypes towards IgM and IgG from B cells and plasma cells, and cytokine production by macrophages (Catalan-Serra et al., 2017; Lilja et al., 2000; Meijer et al., 1979; Mitsialis et al., 2020; Müller et al., 1998; Sieber et al., 1984; Takayama et al., 2010). In the stromal compartment, there is evidence for enhanced vascularization and increased expression of ICAM-1 and MAdCAM-1 by vascular beds, substantial remodeling of collecting lymphatics, and altered migratory potential of fibroblasts (Leeb et al., 2003; Souza et al., 1999). Epithelial barrier dysfunction has also been noted, including alterations to mucus production, microvilli, and Paneth cell dysfunction (Buisine et al., 2001; Stappenbeck and McGovern, 2017). Collectively previous studies have identified that all cell types may be meaningfully altered during CD, and highlight the important need to comprehensively understand the concerted cellular changes that define CD. Importantly, which changes are predictive of more severe disease or treatment resistance in pediCD remain largely unknown.

Massively parallel single-cell RNA-sequencing (scRNA-seq) is enhancing our ability to comprehensively map and resolve the cell types, subsets, and states present during health and disease. This has been particularly evident in the elucidation of novel human cell subsets and states within epithelial, stromal, immune, and neuronal cell lineages. Recent work has generated cellular atlases of previously treated (pre-treated) adult CD and UC, though a comprehensive single-cell atlas for untreated pediatric disease with follow-up of patient outcomes has yet to be reported for IBD (Corridoni et al., 2020a, 2020b; Drokhlyansky et al., 2019; Elmentaite et al., 2020; Huang et al., 2019; Kinchen et al., 2018; Martin et al., 2019; Parikh et al., 2019; Smillie et al., 2019). The potential impact of scRNA-seq on our understanding of IBD is evidenced by studies of adult UC, which have identified potential functional roles for poorly understood colonic cell subsets, such as the BEST4+ enterocyte, and identified pathological alterations in UC pinch biopsies compared to healthy controls, including an expansion of microfold-like cells, IL13RA2+IL11+ inflammatory fibroblasts, CD4+CD8+IL17A+ T cells and CD8+GZMK+ T cells (Smillie et al., 2019). A single-cell study of pre-treated adult CD patients comparing non-inflamed and inflamed tissue from surgically-resected bowel found IgG+ plasma cells, inflammatory mononuclear phagocytes, activated T cells and stromal cells comprising the “GIMATS” pathogenic cellular module (Martin et al., 2019). This module was used to derive a gene signature associated with resistance to anti-TNF therapy in a distinct cohort profiled by bulk RNA-seq. Very recent work has profiled how fetal transcription factors are reactivated in Crohn's disease epithelium (Elmentaite et al., 2020). Together, these and other studies have demonstrated the power of scRNA-seq to nominate individual and collective cell states that are associated with disease, and have also underscored the unmet need to apply these techniques to untreated disease and associate them with disease severity in order to more specifically identify pathognomonic and prognostic cell states.

Citation or identification of any document in this application is not an admission that such a document is available as prior art to the present invention.

### SUMMARY

In one aspect, the present invention provides for a method of treating a subject suffering from inflammatory bowel disease (IBD) comprising: determining whether the subject belongs to a risk group selected from: (i) well controlled without anti-TNF-blockade (NOA), (ii) anti-TNF-blockade full responder (FR), and (iii) anti-TNF-blockade partial responder (PR) by: detecting in a sample obtained from the subject at diagnosis or before treatment the frequency of one or more T cell/Natural Killer/Innate lymphoid cell (T/NK/ILC), myeloid and/or epithelial cell subsets selected from Table 1, determining the risk group of the subject by comparing the frequency of the detected cell subsets to a control frequency for the subsets along a trajectory of disease severity from NOA to FR to PR; and if the subject is in the NOA group, then treating the subject with a treatment that does not comprise anti-TNF-blockade; if the subject is in the FR group, then treating the subject with a treatment comprising anti-TNF-blockade; if the subject is in the PR group, then treating the subject with a treatment comprising anti-TNF-blockade and/or an additional treatment. In certain embodiments, the cell subsets are selected from the group consisting of: CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, CD.Goblet.TFF1.TPSG1, CD.T.LAG3.BATF, CD.T.IFI44L.PTGER4, CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15, wherein the frequency of the CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, and CD.Goblet.TFF1.TPSG1 subsets is increased in PR subjects as compared to NOA subjects, and wherein the frequency of the CD.T.LAG3.BATF, CD.T.IFI44L.PTGER4, and CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15 subsets is decreased in PR subjects as compared to NOA subjects. In certain embodiments, the cell subsets are selected from the group consisting of: CD.NK.MKI67.GZMA, CD.T.MKI67.IL22, CD.Fibro.CCL19.IRF7 and CD.EC.SLC28A2.GSTA2, wherein the frequency of the CD.NK.MKI67.GZMA and CD.T.MKI67.IL22 subsets is increased in FR and PR subjects as compared to NOA subjects, and wherein the frequency of the CD.Fibro.CCL19.IRF7 and CD.EC.SLC28A2.GSTA2 subsets is decreased in FR and PR subjects as compared to NOA subjects. In certain embodiments, the cell subsets are selected from the group consisting of: cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, Mac.DC.CXCL10.CLEC4E, NK.GNLY.FCER1G, T.MKI67.IL22, NK.GNLY.IFNG, EC.OLFM4.MT.ND2, NK.GNLY.GZMB, Mono.Mac.CXCL10.CXCL11, Mono.FCN1.S100A4, T.CARD16.GB2, Mono.CXCL10.TNF, and NK.MKI67.GZMA, wherein the frequency of at least one subset from each of the T/NK/ILLC, myeloid and epithelial cell states subsets is increased in PR subjects as compared to FR and NOA subjects. In certain embodiments, the cell subsets are selected from the group consisting of: CD.EpithStem.LINC00176.RPS4Y1, CD.MCell.CSRP2.SPIB, CD.EC.FABP6.PLCG2, and CD.EC.FABP1.ADIRF, wherein the frequency of the CD.EpithStem.LINC00176.RPS4Y1, CD.MCell.CSRP2.SPIB, CD.EC.FABP6.PLCG2, and CD.EC.FABP1.ADIRF subsets is decreased in FR subjects as compared to NOA subjects. In certain embodiments, the cell subset is the CD.B/DZ.HIST1H1B.MKI67 subset, wherein the frequency of the CD.B/DZ.HIST1H1B.MKI67 subset is increased in PR subjects as compared to FR subjects. In certain embodiments, the anti-TNF-blockade is a monoclonal antibody.

In another aspect, the present invention provides for a method of treating a subject suffering from inflammatory bowel disease (IBD) comprising: detecting in a sample obtained from the subject at diagnosis or before treatment the expression of one or more genes selected from Table 2; determining whether the subject is in the FR or PR risk group by comparing to a control level in FR and/or PR subjects; and if the subject is in the FR group, then treating the subject with a treatment comprising anti-TNF-blockade; if the subject is in the PR group, then treating the subject with a treatment comprising anti-TNF-blockade and/or an additional treatment. In certain embodiments, the one or more genes are detected in one or more cell subsets selected from the group consisting of CD.NK.CCL3.CD160, CD.Fibro.TFPI2.CCL13, CD.Paneth.DEFA6.ITLN2 and CD.Mac.APOE.PTGDS, wherein the one or more cell subsets are detected according to one or more genes in Table 1. In certain embodiments, the one or more genes are selected from the group consisting of IFITM1, APOA1, TPT1, FABP6, NACA, APOA4, MIF, HOPX, SPINK4, CMC1, TNFRSF11B, BRI3, COL1A2, NKG7, APOE, TFPI2, AREG, KLRC1, HTRA3, COL1A1, HIF1A, STAT1, SLC16A4, SERPINE2, CCL11, SAMHD1, TAX1BP1, TXN, GPR65, CEBPB, GSN, EMILIN1, CTNNB1, COL4A1, CLEC12A, PTGER4, BDKRB1, SKIL, and PFN1, wherein APOA1, FABP6, NACA, APOA4, TPT1, SPINK4, MIF, IFITM1, and HOPX are increased in FR relative to PR, and wherein TNFRSF11B, TFPI2, SERPINE2, GSN, COL1A1, HIF1A, COL1A2, CTNNB1, CCL11, EMILIN1, CEBPB, SLC16A4, HTRA3, CMC1, AREG, COL4A1, SKIL, KLRC1, PTGER4, BRI3, APOE, BDKRB1, TXN, GPR65, NKG7, SAMIID1, CLEC12A, STAT1, PFN1, and TAX1BP1 are increased in PR relative to FR. In certain embodiments, the anti-TNF-blockade is a monoclonal antibody.

In another aspect, the present invention provides for a method of treating a subject suffering from inflammatory bowel disease (IBD) comprising: detecting in a sample obtained from the subject at diagnosis or before treatment the expression of one or more genes selected from the group consisting of TNFAIP6, GZMB, S100A8, CSF2, CLEC4E, S100A9, IL1RN, FCGR1A, CLIC3, CD14, PLA2G7, FAM26F, IL3RA, NKG7, IL32, CCL3, OLR1, LILRA4, APOC1, and MYBL2; or Table 14; and if the subject has decreased expression of the one or more genes compared to a control, then treating the subject with a treatment comprising anti-TNF-blockade; if the subject has increased expression of the one or more genes compared to a control, then treating the subject with a treatment comprising anti-TNF-blockade and/or an additional treatment. In certain embodiments, the anti-TNF-blockade is a monoclonal antibody.

In another aspect, the present invention provides for a method of stratifying subjects suffering from IBD into a risk group comprising detecting in a sample obtained from a subject at diagnosis or before treatment the frequency of one or more T cell/Natural Killer/Innate lymphoid cell (T/NK/ILC), myeloid and/or epithelial cell subsets selected from Table 1, and determining if the subject is in a well-controlled without anti-TNF-blockade (NOA) risk group, an anti-TNF-blockade full responder (FR) risk group, or anti-TNF-blockade partial responder (PR) risk group of the subject by comparing the frequency of the detected cell subsets to a control frequency for the subsets along a trajectory of disease severity from NOA to FR to PR. In certain embodiments, the cell subsets are selected from the group consisting of: CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, CD.Goblet.TFF1.TPSG1, CD.T.LAG3.BATF, CD.T.IFI44L.PTGER4, CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15, wherein the frequency of the CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, and CD.Goblet.TFF1.TPSG1 subsets is increased in PR subjects as compared to NOA subjects, and wherein the frequency of the CD.T.LAG3.BATF, CD.T.IFI44L.PTGER4, and CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15 subsets is decreased in PR subjects as compared to NOA subjects. In certain embodiments, the cell subsets are selected from the group consisting of: CD.NK.MKI67.GZMA, CD.T.MKI67.IL22, CD.Fibro.CCL19.IRF7 and CD.EC.SLC28A2.GSTA2, wherein the frequency of the CD.NK.MKI67.GZMA and CD.T.MKI67.IL22 subsets is increased in FR and PR subjects as compared to NOA subjects, and wherein the frequency of the CD.Fibro.CCL19.IRF7 and CD.EC.SLC28A2.GSTA2 subsets is decreased in FR and PR subjects as compared to NOA subjects. In certain embodiments, the cell subsets are selected from the group consisting of: cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, Mac.DC.CXCL10.CLEC4E, NK.GNLY.FCER1G, T.MKI67.IL22, NK.GNLY.IFNG, EC.OLFM4.MT.ND2, NK.GNLY.GZMB, Mono.Mac.CXCL10.CXCL11, Mono.FCN1.S100A4, T.CARD16.GB2, Mono.CXCL10.TNF, and NK.MKI67.GZMA, wherein the frequency of at least one subset from each of the T/NK/ILC, myeloid and epithelial cell states subsets is increased in PR subjects as compared to FR and NOA subjects. In certain embodiments, the cell subsets are selected from the group consisting of: CD.EpithStem.LINC00176.RPS4Y1, CD.MCell.CSRP2.SPIB, CD.EC.FABP6.PLCG2, and CD.EC.FABP1.ADIRF, wherein the frequency of the CD.EpithStem.LINC00176.RPS4Y1, CD.MCell.CSRP2.SPIB, CD.EC.FABP6.PLCG2, and CD.EC.FABP1.ADIRF subsets is decreased in FR subjects as compared to NOA subjects. In certain embodiments, the cell subset is the CD.B/DZ.HIST1H1B.MKI67 subset, wherein the frequency of the CD.B/DZ.HIST1H1B.MKI67 subset is increased in PR subjects as compared to FR subjects. In certain embodiments, the IBD is Crohn's Disease (CD).

In another aspect, the present invention provides for a method of stratifying subjects suffering from IBD into a risk group comprising: detecting in a sample obtained from a subject at diagnosis or before treatment the expression of one or more genes selected from the group consisting of TNFAIP6, GZMB, S100A8, CSF2, CLEC4E, S100A9, IL1RN, FCGR1A, CLIC3, CD14, PLA2G7, FAM26F, IL3RA, NKG7, IL32, CCL3, OLR1, LILRA4, APOC1, and MYBL2; or Table 14, and determining if the subject is in a well-controlled without anti-TNF-blockade (NOA) risk group, an anti-TNF-blockade full responder (FR) risk group, or anti-TNF-blockade partial responder (PR) risk group by comparing the expression of the one or more genes to a control expression for the subsets along a trajectory of disease severity from NOA to FR to PR. In certain embodiments, the IBD is Crohn's Disease (CD).

In certain embodiments, the cell states or genes are detected by RNA-seq, immunohistochemistry (IHC), fluorescently bar-coded oligonucleotide probes, RNA FISH, FACS, or any combination thereof. In certain embodiments, the cell states are inferred from bulk RNA-seq. In certain embodiments, the cell states are determined by single cell RNA-seq. In certain embodiments, the sample is obtained by biopsy. In certain embodiments, the subject is younger than 35, 25, 20, or 18 years old. In certain embodiments, when the frequency of a cell state increases, the frequency of a cell state in the parent cells for the control subject is less than 0, 5, 10, or 50 percent of the parent cell. In certain embodiments, when the frequency of a cell state decreases, the frequency of a cell state in the parent cells for the control subject is greater than 0, 5, 10, or 50 percent of the parent cell.

In certain embodiments, the CD.NK.MKI67.GZMA cell state is detected by detecting one or more genes selected from the group consisting of GNLY, CCL3, KLRD1, IL2RB and EOMES. In certain embodiments, the CD.T.MKI67.IL22 cell state is detected by detecting one or more genes selected from the group consisting of IFNG, CCL20, IL22, IL26, CD40LG and ITGAE. In certain embodiments, the CD.Fibro.CCL9.IRF7 cell state is detected by detecting one or more genes selected from the group consisting of CCL19, CCL11, CXCL1, CCL2, OAS1 and IRF7. In certain embodiments, the CD.EC.SLC28A2.GSTA2 cell state is detected by detecting one or more genes selected from the group consisting of SLC28A2 and GSTA2. In certain embodiments, the CD.T.MKI67.IFNG cell state is detected by detecting one or more genes selected from the group consisting of IFNG, GNLY, HOPX, ITGAE and IL26. In certain embodiments, the CD.T.MKI67.FOXP3cell state is detected by detecting one or more genes selected from the group consisting of IL2RA, BATF, CTLA4, TNFRSF1B, CXCR3, and FOXP3. In certain embodiments, the CD.T.GNLY.CSF2 cell state is detected by detecting one or more genes selected from the group consisting of GNLY, GZMB, GZMA, PRF1, IFNG, CXCR6, and CSF2. In certain embodiments, the CD.NK.GNLY.FCER1G cell state is detected by detecting one or more genes selected from the group consisting of GNLY, GZMB, GZMA, PRF1, AREG, TYROBP, and KLRF1. In certain embodiments, the CD.Mac.CXCL3.APOC1 cell state is detected by detecting one or more genes selected from the group consisting of CCL3, CCL4, CXCL3, CXCL2, CXCL1, CCL20, CCL8, TNF and IL1B. In certain embodiments, the CD.Mono/Mac.CXCL10.FCN1 cell state is detected by detecting one or more genes selected from the group consisting of CXCL9, CXCL10, CXCL11, GBP1, GBP2, GBP4, GBP5, and Type II IFN-gamma. In certain embodiments, the CD.Mono.FCN1.S100A4 cell state is detected by detecting one or more genes selected from the group consisting of S100A4, S100A6, and FCN1.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1E—Study design with patient diagnosis, criteria and histopathology. FIG. 1a, Schematic showing cohorts, analysis of cells by flow cytometry and analysis of cells by single cell RNA sequencing. FIG. 1b, Demographics of cohorts. FIG. 1c, Clinical parameters. FIG. 1d, histopathology. FIG. 1e, Treatment response grading.

FIG. 2A-2B—Flow cytometry does not reveal significant changes in FGID vs CD or across the CD treatment response spectrum. FIG. 2a, Flow cytometry of leukocytes, monocytes and Natural Killer cells in CD and FGID samples. FIG. 2b, Flow cytometry of dendritic cells, plasmacytoid dendritic cells and T cells in CD and FGID samples.

FIG. 3A-3E—A comprehensive atlas of terminal ileum in non-inflammatory FGID. FIG. 3a, Force-directed/UMAP layout for all cell types. FIG. 3b, UMAP layout for each patient individually. FIG. 3c, UMAP layout for each cell type individually. FIG. 3d, Taxonomy w/ subset and donor distribution. FIG. 3e, Dot-Plot for some top genes that help classify each of the overarching cell types.

FIG. 4A-4E—A comprehensive atlas of terminal ileum in Crohn's disease. FIG. 4a, Force-directed/UMAP layout for all cell types. FIG. 4b, UMAP layout for each patient individually. FIG. 4c, UMAP layout for each cell type individually. FIG. 4d, Taxonomy w/ subset and donor distribution. FIG. 4e, Dot-Plot for some top genes that help classify each of the overarching cell types.

FIG. 5A-5E—PCA of cell composition in pediCD reveals predictive axes of disease trajectory and treatment response. FIG. 5a, Spearman rank clustered heatmap. FIG. 5b, Volcano plots of T/NK/TLC cell cluster composition. FIG. 5c, Volcano plots of myeloid cell cluster composition. FIG. 5d, Graphs showing indicated cell cluster frequency of parent cell type in NOA, responders and partial responders. FIG. 5e, Graph showing correlation of T/NK/ILC and Myeloid and Epithelial each with PC2 with anti-TNF_NOA_FR_PR (r=−0.87).

FIG. 6A-6F—Random Forest Classifier applied to cellular taxonomies reveals changes in cell state composition across disease severity spectrum (Correspondence, Bias, Hierarchy, NOA vs FR vs PR). FIG. 6a, B cells. FIG. 6b, Endothelial cells. FIG. 6c, Epithelial cells. FIG. 6d, Fibroblasts. FIG. 6e, Myeloid cells. FIG. 6f, T cells.

FIG. 7A-7E—Pseudotime over a shared gene expression space of the T/NK/ILCs. FIG. 7a, T cell “deep dive” pseudotime. FIG. 7b, Genes that correspond with specific subsets of interest. FIG. 7c-e, Quantification of the overall differences in distribution of FGID, NOA, FR and PR.

FIG. 8A-8G—Pseudotime over a shared gene expression space of the monocytes/macrophages. FIG. 8a, Macrophage “deep dive” pseudotime. FIG. 8b, Genes that correspond with specific subsets of interest. FIG. 8c-e, Quantification of the overall differences in distribution of FGID, NOA, FR and PR. FIG. 8f, TNF expression in specific subtypes in FGID, NOA, FR and PR across pseudotime. FIG. 8g, Heatmap showing single cell gene expression in chemokine macrophages and resting macrophages.

FIG. 9A-9C—Medication timelines for all patients in CD cohorts. FIG. 9a, Full responders (FR). FIG. 9b, Partial responders (PR). FIG. 9c, Not on anti-TNF (NOA).

FIG. 10A-10E—PREDICT Study Design with Patient Diagnostic Criteria and Histopathology. FIG. 10a, Study overview depicting clinical and cellular measurements from 13 functional gastrointestinal disorder (FGID) patients and 14 pediatric Crohn's disease (pediCD) patients. Terminal ileum biopsies were isolated at a treatment-naïve diagnostic visit, and pediCD patients were followed up to determine their anti-TNF response and categorized as not on anti-TNF (NOA), Full Response (FR), or Partial Response (PR) (see Methods). Two panels of flow cytometry allowed for relative frequency quantification of 32 cell types and subsets, and 10×3′ v2 single-cell RNA-sequencing (scRNA-seq) captured 245,911 total cells including 138 FGID and 305 pediCD end clusters through an iterative tiered clustering (ITC) approach, ARBOL (see Methods). FIG. 10b, Demographic data, weight, height, and BMI for cohort (see Table 5 and FIG. 18. FIG. 10c, Clinical inflammatory laboratory values for cohort (see Table 5 and FIG. 18). FIG. 10d, Representative histopathology of FGID (top) and pediCD (bottom) at 10× (scale bar=100 um) and 40× (scale bar=20 um) magnification. FIG. 10e, Representative treatment history and clinical inflammatory parameters used for determination of NOA, FR and PR status (see Methods, Table 5, and FIG. 18; ADA: adalimumab, INF: infliximab; MTX: methotrexate; Pred: prednisone; mSCD: modified specific carbohydrate diet; EEN: exclusive enteral nutrition).

FIG. 11A-11E—Flow Cytometry of Ileal Biopsies Does Not Reveal Significant Changes in Cell Composition in FGID vs. pediCD or across the pediCD Treatment Response Spectrum. FIG. 11a, Representative flow cytometry end gates for selected cell subsets (left: epithelial and hematopoietic; middle: naïve and effector T cells; right: pDCs and antigen-presenting cells) from single-cell dissociated samples from one terminal ileum biopsy for pediCD patients (see FIG. 19 for full gating strategy). FIG. 11b, Fractional composition of selected cell subsets of CD45+ cells from 13 FGID and 14 pediCD patients (error bars are s.e.m). FIG. 11c, Fractional composition of selected cell subsets of CD45+ cells from 4 NOA, 5 FR and 5 PR patients. FIG. 11d, Fractional composition of dendritic, pDC, central memory (CM) and effector memory (EM) CD4+ and CD8+ cells from 13 FGID vs 14 pediCD patients. Dendritic cells and pDC plotted as percentage of CD45+ cells. CM/EM CD4+ and CD8+ cells plotted as percentage of total CD4+ and CD8+ cells, respectively. p<0.05 by Mann-Whitney for pediCD versus FGID and 1-way ANOVA for pediCD cohorts). FIG. 11e, Fractional composition of dendritic cells, pDCs, central memory (CM) and effector memory (EM) CD4+ and CD8+ cells from 4 NOA, 5FR, and 5 PR patients. Graphs plotted as in d.

FIG. 12A-12E—A Comprehensive Cell Atlas of Terminal Ileum in Non-Inflammatory FGID. FIG. 12a, tSNE of 99,488 single-cells isolated from terminal ileal biopsies of 13 FGID patients. Colors represent major cell type groups determined via Louvain clustering with resolution set by optimized silhouette score. FIG. 12b, tSNE as in a with individual patients plotted. For specific proportions please see FIG. 21. FIG. 12c, tSNE of each major cell type which was used as input into iterative tiered clustering (ITC). FIG. 12d, Hierarchical clustering of complete FGID data set with input clusters determined based on results of ITC and performed on the median expression of 4,428 pairwise differentially expressed genes, using complete linkage and distance calculated with Pearson correlation, between each end cell cluster. Simpson's Index of Diversity represented as 1-Simpson's where 1 (black) indicates equivalent richness of all patients in that cluster, and 0 (white) indicates a completely patient-specific subset. Numbers represent the number of cells in that cluster. Names of subsets are determined by Disease.CellType.GeneA.GeneB as in Methods. FIG. 12e, Dot plot of 2 defining genes for each cell type. Dot size represents fraction of cells expressing the gene, and intensity represents binned count-based expression level (log(scaled UMI+1)) amongst expressing cells. Cluster defining genes are provided in Table 4.

FIG. 13A-13E—A Comprehensive Cell Atlas of Terminal Ileum in pediCD. FIG. 13a, tSNE of 124,054 single-cells isolated from terminal ileal biopsies of 14 pediCD patients. Colors represent major cell type groups determined via Louvain clustering with resolution set by optimized silhouette score. FIG. 13b, tSNE as in a with individual patients plotted. For specific proportions please see FIG. 21. FIG. 13c, tSNE of each major cell type which was used as input into iterative tiered clustering (ITC). FIG. 13d, Hierarchical clustering of complete pediCD data set with input clusters determined based on results of ITC, and performed on the median expression of 4,428 pairwise differentially expressed genes, using complete linkage and distance calculated with Pearson correlation, between each end cell cluster. Simpson's Index of Diversity represented as 1-Simpson's where 1 (black) indicates equivalent richness of all patients in that cluster, and 0 (white) indicates a completely patient-specific subset. Numbers represent the number of cells in that cluster. Names of subsets are determined by Disease.CellType.GeneA.GeneB as in Methods. FIG. 13e, Dot plot of 2 defining genes for each cell type. Dot size represents fraction of cells expressing the gene, and intensity represents binned count-based expression level (log(scaled UMI+1)) amongst expressing cells. Cluster defining genes are provided in Table 1.

FIG. 14A-14D—A Collective Cell Vector in pediCD Reveals Predictive Axes of Disease Trajectory and Treatment Response. FIG. 14a, Spearman rank correlation heatmap of principal components calculated from the frequencies of each end cluster per main cell type together with clinical metadata. Correlation is represented by both the intensity and size of the box and those which are FDR <0.05 have a bounding box (inset highlights the specific correlation between PC2 of the T, Myeloid, Epithelial cell frequency analysis with anti-TNF response). FIG. 14b, Volcano plots for T/NK/ILC and myeloid cell clusters between NOA, FR and PR, where named clusters are significant by Fisher's exact test and those in pink are significant by Mann-Whitney U test. FIG. 14c, Cell cluster frequencies of the parent cell type found to be significant by Mann-Whitney U test between selected clusters (see FIG. 24 for all graphs; Table 12). FIG. 14d, Heatmap showing cell frequencies per patient of most positive and negative cell subsets of PC2 from PCA performed on T/NK/ILC, myeloid and epithelial cell subsets (Table 13). Cell subsets are sorted by PC2 score, and patients were sorted by anti-TNF response. Heatmap is not normalized and displaying the log counts-per million of each cell subset normalized per cell type. *Patient p022's response category changed from FR to PR after database lock in December of 2020. No other patient's categorization has changed.

FIG. 15A-15F—Random Forest (RF) Classifier Applied to Myeloid Cellular Taxonomies Identifies Correspondence between FGID and pediCD. FIG. 15a, Correspondence between cell subsets from FGID-to-pediCD and pediCD-to-FGID. Top left heatmaps: RF probabilities for each cell averaged over subset to gain probability of each FGID matching onto each pediCD subset (left), and pediCD onto FGID (right). Bubble plot (center): size=sum(probability matrices) for confidence of predictions, marker color=diff(probability matrices) to show which direction the RF model is more confident on, e.g. more likely for FGID subset to belong to pediCD subset or pediCD subset to belong to FGID subset. Markers are filtered to show the top 10^{th} percentile of correspondence. Dendrograms: separated-tiered clustering on prediction probabilities of FGID (blue) and pediCD (red) using complete linkage with correlation distance metric, clusters are cut at height 0.7 (range 0-1). Heatmap: 1-Gini-Simpson index based on patient diversity, mono-patient clusters (white), full representation (black). Right 3 columns show row-normalized of frequency of NOA, FR, PR representation in each CD cell subset. Significant differences (Mann-Whitney, alpha=0.05) are marked, triangle NOA vs. PR and circle NOA vs. FR. FIG. 15b, Distribution of Gini-Simpson's index of patient diversity in FGID (top) and pediCD (bottom) for myeloid cell clusters. FIG. 15c, Sankey plot comparing joined traditional single-level clustering (left) to disease-separated iterative tiered clustering (right). Each line follows each cell as it moves between in the two cluster sets (back bar split based on cluster identity). FIG. 15d, Gini-Simpson index on representation of traditional clusters in each of the separated tiered clusters (i.e., from how many of the higher-level clusters does the deep clustering pull). Calculated separately for FGID (blue) and pediCD (red). FIG. 15e, Similar to d but showing the total counts of how many traditional clusters are represented in a single tiered cluster per disease. FIG. 15f, UMAP of combined Myeloid cells: red shows example end clusters from ITC that are split across the traditional-clustering joint-disease UMAP.

FIG. 16A-16G—Distinct Distributions of Macrophages Across the pediCD Treatment Response Spectrum Relative to FGID. FIG. 16a, UMAP representation of macrophages (27 patients; 10,134 cells) from FG and pediCD datasets, run across 50 principal components based on 539 genes significantly upregulated (Wilcoxon; p.adj<0.05) in macrophages versus all other cell types and not significantly differentially expressed between FG and pediCD sets. UMAP parameters [min-dist=0.1, N-neighbors=ceiling(sqrt(Ncells)/2)]. Labels are set at median of IQR for each subset. Clinical metadata showing future response to anti-TNF treatment: FGID (grey), NOA (blue), FR (yellow), PR (red). FIG. 16b, Same UMAP as in a colored to isolate single subsets. Subsets chosen based on significant Mann-Whitney tests (FIG. 5; Supplemental FIG. 7), (black) cells from subset, (grey) rest of macrophages. FIG. 16c, Same UMAP as in a separated into FGID and pediCD. FIG. 16d, Same UMAP as in a split into each treatment response group. Shaded area captures 80% most densely populated regions of plot area calculated using 2d KDE estimate from MASS R package. FIG. 16e, Permutation test results using Hellinger distance to measure if 2 conditions are sampled from the same distribution (0=complete overlap, 1=no overlap). Hellinger distance is computed with sqrt(1−sum(sqrt(kdel*kde2))) with a KDE estimation for each condition group calculated across 1000 points uniformly distributed across plot area, with bandwidth selected using ks::Hpio function. Black distribution shows test statistic varying min-dist parameter with 11 evenly spaced values between 0.01 and 1. Vertical line shows test statistic using UMAP parameters [min-dist=0.1, Neighbors=ceiling(sqrt(Ncells)), Npcs=50, nDim=2]. Grey distribution shows results of 11,000 permutations to treatment response group varied across same min-dist umap parameters between 0.01 and 1. All tests are significant beyond a 0.001 threshold. FIG. 16f, Clockwise from left: UMAP of macrophages with color intensity displaying amount of TNF expression based on ((log(scaledUMI+1)). Plot (top) showing fraction of macrophages expressing TNF with colored dots showing fraction of TNF+ cells within each treatment response group and grey violins showing results of 10,000 permutations of treatment response labels. Violin plot (bottom) of ((log(scaledUMI+1)) TNF expression split on treatment response group. FIG. 16g, Diversity of macrophage clusters in FGID and pediCD: (top) each dot represents a cell subset, y-axis shows how many patients are included within the subset. (bottom) each dot represents a subset, with y position showing (1-Gini-Simpson's Diversity Index), Subsets below red dashed line set at 0.1 diversity were excluded.

FIG. 17A-17G—Distinct Distributions of Lymphocytes Across the pediCD Treatment Response Spectrum Relative to FGID. FIG. 17a, UMAP representation of T/NK/ILCs (27 patients; 67,579 cells) from FG and CD datasets, run across 50 principal components based on 345 genes significantly upregulated (Wilcoxon; p.adj<0.05) in lymphocytes versus all other cell types and not significantly differentially expressed between FG and pediCD sets. UMAP parameters [min-dist=0.1, N-neighbors=ceiling(sqrt(Ncells)/2)] Labels are set at median of IQR for each subset. Clinical metadata showing future response to anti-TNF treatment: FGID (grey), NOA (blue), FR (yellow), PR (red). FIG. 17b, Same UMAP as in a colored to isolate single subsets. Subsets chosen based on significant Mann-Whitney tests (FIG. 5), (black) cells from subset, (grey) rest of lymphocytes. FIG. 17c, Same UMAP as in a separated into FG and pediCD. FIG. 17d, Same UMAP as in a split into each treatment response group. Shaded area captures 80% most densely populated regions of plot area calculated using 2d KDE estimate from MASS R package. FIG. 17e, Permutation test results using Hellinger distance to measure if 2 conditions are sampled from the same distribution (0=complete overlap, 1=no overlap). Hellinger distance is computed with sqrt(1−sum(sqrt(kdel*kde2))) with a KDE estimation for each condition group calculated across 1000 points uniformly distributed across plot area, with bandwidth selected using ks::Hpio function. Black distribution shows test statistic varying min-dist parameter with 11 evenly spaced values between 0.01 and 1. Vertical line shows test statistic using UMAP parameters [min-dist=0.1, Neighbors=ceiling(sqrt(Ncells)), Npcs=50, nDim=2]. Grey distribution shows results of 11,000 permutations to treatment response group varied across same min-dist umap parameters between 0.01 and 1. All tests are significant beyond a 0.001 threshold. FIG. 17f, Violin plot (left) of ((log(scaledUMI+1))MKI67 expression split on treatment response group. UMAP (right) of lymphocytes with color intensity displaying MKI67 expression based on ((log(scaledUMI+1)) (right). FIG. 17g, Diversity of lymphocyte clusters in FGID and CD: (top) each dot represents a cell subset, y-axis shows how many patients are included within the subset. (bottom) each dot represents a subset, with y position showing (1-Gini-Simpson's Diversity Index), Subsets below red dashed line set at 0.1 diversity were excluded.

FIG. 18—Clinical trajectory and treatments for all pediCD patients. Representative treatment history and clinical inflammatory parameters used for determination of NOA, FR and PR status for all pediCD patients (see Methods, Table 5, and FIG. 1; ADA: adalimumab, INF: infliximab; MES: mesalamine MTX: methotrexate; Pred: prednisone; mSCD: modified specific carbohydrate diet; EEN: exclusive enteral nutrition).

FIG. 19A-19B—Representative gating strategies for flow cytometry. FIG. 19a, Representative gating strategy for Panel 1 focused on T cells and myeloid cells, for antibodies see Table 7. FIG. 19b Representative gating strategy for Panel 2 focused on non-classical T cells and innate lymphoid cells (NB: Lineage=CD14, CD20, CD11c, CD11b, CD56), for antibodies see Table 7.

FIG. 20A-20C—Comparison of quality control measures reveals similar sequencing depths and gene capture between FGID and pediCD. FIG. 20a, Quality control measures for scRNA-seq of ileal biopsies of 27 patients (13 FGID, 14 pediCD) included in the study. Top two graphs denote total genes (nFeature) and UMIs (nCount) after normalization with SCTransform. Lower graphs denote total genes (nFeature), UMIs (nCount) and mitochondrial read percentage (mt.percentage) of pre-processed 10×3′ v2 single-cell RNA-sequenced samples. FIG. 20b, Quality control measures as in a split by cell type. FIG. 20c, Comparison of total genes captured (nFeature, left) and total UMIs (nCount, right) between FGID (blue) and pediCD (red) split by cell type.

FIG. 21A-21G—Traditional clustering with SCTransform normalization reveals similarities across cell types in FGID and pediCD. FIG. 21a, UMAPs representing one round of clustering of 197,281 single-cells across FGID and pediCD samples. Traditional clustering performed on disease states together. Colors represent major cell types determined by one round of clustering with Seurat RunUMAP parameters (PCs=1:50, n.neighbors=50, min.dist=1). Cell types were assigned based on significantly upregulated marker genes (Wilcoxon; p.adj<0.05) obtained from comparison of specific cell type versus all other cell types. FIG. 21b, UMAPs as in a colored to highlight FGID (blue) and pediCD (red) cells. FIG. 21c, UMAP as in a colored by Tier 1 ITC clusters performed separately for FGID and pediCD. FIG. 21d, Comparison of cell cluster frequencies between FGID (blue) and pediCD (red). Patient contributions denoted by circles (FGID) and triangles (pediCD). FIG. 21e, Differentially expressed genes across cell type in FGID vs pediCD determined to be significant by Wilcoxon test (log FC>0.25, FDR<0.001). FIG. 21f, Volcano plots for Myeloid, Epithelial, T-cell clusters denoting differentially expressed genes in FGID vs. pediCD. Those in pink are significant by Wilcoxon test. FIG. 21g, UMAPs of jointly clustered pediCD and FGID Myeloid cells.

FIG. 22A-22C—Schematic for iterative tiered clustering and random forest classifier approach. FIG. 22a, Flowchart depicting iterative tiered clustering (ITC) used for generating FGID and pediCD cellular atlases. After sequencing, cells underwent quality control and a cell by gene expression matrix was derived from the 27 ileal samples. Dimensionality reduction and graph-based clustering were performed using the standard Seurat workflow to annotate cell types. Resulting clusters were then iteratively processed through the same pipeline unless end conditions were met. Each cluster was checked for three end conditions which included: only one cluster remaining, two clusters remaining with no more than 5 up and down regulated genes as determined by Wilcoxon test (log FC>1.5, FDR<0.001), and/or less than 100 cells in the cluster. Iterative clustering stopped if any of the three conditions are met. Unlike traditional Seurat clustering, in ITC principal component and clustering resolution parameters are chosen automatically. Stop conditions are built in as parameters to the ITC pipeline, allowing customization to the dataset. FIG. 22b, Cell and cluster numbers after various processing steps tabulated. FIG. 22c, Random forest classifier approach for integrating FGID and pediCD datasets. FGID and pediCD datasets were used as training datasets to create random forest predictors used in downstream sub-clustering of cell types and subsets. The opposing dataset was then tested by each algorithm independently to determine correspondence and bias as depicted in FIG. 15 and FIG. 25.

FIG. 23A-23B—Representative marker genes for myeloid and T cells. FIG. 23a, Dot plot of curated genes related to myeloid biology. Dot size represents fraction of cells expressing the gene, and color intensity represents binned count-based expression level (log(scaled UMI+1)) amongst expressing cells. Cluster defining genes are provided in Table 1 and Table 4. Dot size is only plotted if more than 5% of cells are expressing the transcript. Names are descriptive names generated from inspection of ITC output which were then converted to standardized naming scheme as in Methods. FIG. 23b, Dot plot of marker genes related to T/NK/ILC lymphoid biology as in a.

FIG. 24A-24E—Cell types associated with pediCD severity after PCA analysis. FIG. 24a, Cell cluster frequencies of the parent cell type found to be significant by Mann-Whitney U test between selected clusters. FIG. 24b, Cell cluster frequencies of the parent cell type between NOA and FR (as above). FIG. 24c, Cell cluster frequencies of the parent cell type between NOA and PR (as above). FIG. 24d, Cell cluster frequencies of the parent cell type between FR and PR (as above). FIG. 24e, GSEA analysis showing the ranks of 92 PREDICT markers (markers of top 25 cell states associated with disease severity and treatment outcomes) in bulk RNA sequencing of illeal or colonic mucosa of two other treatment-naïve cohorts (pediatric RISK cohort, n=69, adult E-MTAB-7604 cohort, n=43) comparing pediCD patients who did or did not respond to anti-TNF therapy. P-value is estimated based on an adaptive multi-level split Monte-Carlo scheme.

FIG. 25A-25C—Random Forest classification applied to T cell subsets and integration using STACAS. FIG. 25a, Correspondence between cell subsets from FGID-to-pediCD and pediCD-to-FGID. Top left heatmaps: RF probabilities for each cell averaged over subset to gain probability of each FGID matching onto each pediCD subset (left), and pediCD onto FGID (right). Bubble plot (center): size=sum(probability matrices) for confidence of predictions, marker color=diff(probability matrices) to show which direction the RF model is more confident on, e.g. more likely for FGID subset to belong to pediCD subset or pediCD subset to belong to FGID subset. Markers are filtered to show the top 10^{th} percentile of correspondence. Dendrograms: separated-tiered clustering on prediction probabilities of FGID (blue) and pediCD (red) using complete linkage with correlation distance metric, clusters are cut at height 0.7 (range 0-1). Heatmap: 1-Gini-Simpson index based on patient diversity, mono-patient clusters (white), full representation (black). Right 3 columns show row-normalized of frequency of NOA, FR, PR representation in each pediCD cell subset. Significant differences (Mann-Whitney, alpha=0.05) are marked, triangle NOA vs. PR and circle NOA vs. FR. FIG. 25b, T cells from the main FGID (n=29,640 cells) and pediCD (n=38,031) datasets were integrated using identification of mutual nearest neighbors in a reduced space (reciprocal PCA method) with the STACAS package. UMAP plots show distribution of cells coming from FGID (blue) and pediCD (red) datasets and 11 clusters obtained using Louvain algorithm. Sankey plot shows the contribution of ARBOL clusters to each Louvain cluster in the integrated dataset. FIG. 25c, Spearman rank correlation heatmap of the counts-per-million for each of the top 25 clusters defining PC2 positive (NOA-associated) and PC2 negative (PR-associated) vectors. Correlation is represented by both the intensity and size of the box and those which are FDR<0.05 have a bounding box.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of treating IBD based on detection of specific cell types, subsets, and states in the subject that indicate whether the subject will respond to anti-TNF-blockade. Single-cell approaches are transforming our ability to understand the barrier tissue biology of inflammatory diseases. Crohn's disease is an inflammatory bowel disease (IBD) which most often presents with patchy lesions in the terminal ileum and proximal colon and requires complex clinical care. Recent advances in the targeting of cytokines and leukocyte migration have greatly advanced treatment options, but most patients still relapse and inevitably progress. As comprehensive single-cell RNA-sequencing (scRNA-seq) atlases of IBD to date have been conflated by sampling treated patients with established disease, there is a lack of a rigorous understanding of which cell types, subsets, and states at diagnosis are predictive of disease severity and response to treatment. Here, through the combined clinical, flow cytometric, and single-cell RNA-sequencing study, Applicants profile primary human biopsies from the terminal ileum of treatment-naïve pediatric patients with non-inflammatory functional gastrointestinal disorder (FGID; n=13) or Crohn's disease (pediCD; n=14). Applicants report transcriptomes of 201,883 cells which enabled for deploying a principled and unbiased tiered clustering approach, ARBOL, to fully resolve and annotate epithelial, stromal, and immune cell states, yielding 138 FGID and 305 pediCD end cell clusters. Thus, Applicants have generated a single cell pediatric Crohn's disease (pediCD) and FGID atlas. Notably, through both flow cytometry and scRNA-seq, Applicants observe that at the level of broad cell types, treatment-naïve Crohn's disease (pediCD) does not significantly vary from FGID in cellular composition. However, by using the high-resolution scRNA-seq analysis, Applicants identified significant differences in cell states that arise during Crohn's disease relative to FGID. Furthermore, by closely linking the scRNA-seq analysis with clinical meta-data, Applicants resolved a vector of T/NK/ILC (lymphoid), myeloid, and epithelial cell states in treatment-naïve samples which can distinguish patients with less severe disease (those not on anti-TNF therapies (NOA)), from those with more severe disease at presentation who require anti-TNF therapies. Moreover, this vector was also able to distinguish those patients that achieve a full response (FR) to anti-TNF blockade from those more treatment-resistant patients who only achieve a partial response (PR). Applicants find significant changes in cell states across all cell types in PRs relative to NOAs and FRs, highlighting cytotoxic lymphocytes (NK.MKI67.GZMA, NK.GNLY.FCER1G), substantial remodeling of the myeloid compartment (Mono.FCN1.S100A4, Mono/Mac.CXCL10.FCN1, Mac.CXCL3.APOC1) and shifts in epithelial cell phenotypes (Goblet.RETNLB.ITLN1, EC.NUPR1.LCN2) associated with increased disease severity and anti-TNF treatment non-response. Cell subsets described further herein are defined by the specific cell states identified and the terms can be used interchangeably. This study jointly leverages a treatment-naïve cohort, high-resolution principled scRNA-seq data analysis, and clinical outcomes to understand which baseline cell states can be used to predict inflammatory disease trajectory. Thus, the present invention advantageously provides for predicting patient response in IBD. Applicants provide a first treatment naïve atlas from any inflammatory disease. Applicants identify cell states specific in severe ileal Crohn's. Baseline cell states are disclosed that can predict treatment response and non-response in IBD. Applicants provide for novel analysis methods.

As used herein, the terms “NOA” or “Not On Anti-TNE” refers to a subject having biopsy-proven pediCD, but for whom clinical symptoms were sufficiently mild that the treating physician did not prescribe anti-TNF agents. NOA can also refer to subjects in which anti-TNF therapy is not necessary. As used herein, the terms “FR” and “full responder” refers to a subject having pediCD and treated with anti-TNF agents who achieved a full response (FR). FR can also refer to subjects in which anti-TNF therapy may succeed in controlling disease. As used herein, the terms “PR” and “partial responder” refers to a subject having pediCD and treated with anti-TNF agents who achieved a partial response (PR). PR can also refer to subjects in which subjects will either immediately or progressively gain resistance to anti-TNF therapy. PR can also refer to subjects that will not succeed in controlling disease. As used herein, “controlling disease” refers to clinical symptom control and biochemical response (measuring CRP, ESR, albumin, and complete blood counts (CBC)), and with a weighted Pediatric Crohn's Disease Activity Index (PCDAI) score of <12.5 on maintenance anti-TNF therapy with no dose adjustments required (Cappello and Morreale, 2016; Hyams et al., 1991; Sandborn, 2014; Turner et al., 2012, 2017). FR can be defined as clinical symptom control and biochemical response. PR to anti-TNF therapy can be defined as a lack of full clinical symptom control as determined by the treating physician or lack of full biochemical response, with documented escalation of anti-TNF therapy or addition of other agents.

### Therapeutic and Diagnostic Methods

In certain embodiments, shifts in cell types or subsets of a cell type are used to predict a disease state and for selecting a treatment. In certain embodiments, shifts in cell states in cell types or subsets of a cell type and are used to predict a disease state and for selecting a treatment. As used herein, cell state refers to the differential expression of genes in specific cell subsets. As used herein, gene expression is not limited to mRNA expression and may also include protein expression. In certain embodiments, the cell subset frequency and/or cell states can be detected for screening novel therapeutics. The present invention, provides for subsets of cell types in CD and FGID. In certain embodiments, the frequency of the cell subsets are shifted in disease states. Disease states may include disease severity or response to any treatment in the standard of care for the disease. In certain embodiments, the disease is an inflammatory disease. In certain embodiments, the inflammatory disease is a disease of a barrier tissue. As used herein a “barrier cell” or “barrier tissues” refers generally to various epithelial tissues of the body such, but not limited to, those that line the respiratory system, digestive system, urinary system, and reproductive system as well as cutaneous systems. The epithelial barrier may vary in composition between tissues but is composed of basal and apical components, or crypt/villus components in the case of intestine.

In certain embodiments, disease states or conditions are treated, monitored or detected. In certain embodiments, diseases relevant to the present invention are inflammatory diseases of a barrier tissue. In certain embodiments, the cell subset composition or frequency and cell states are shifted in any such inflammatory disease. In certain embodiments, detection of specific cell subsets and/or cell states indicates whether the disease can be treated with anti-TNF blockade. Exemplary diseases include, but are not limited to inflammatory bowel disease (IBD) including Crohn's disease (CD) and ulcerative colitis (UC), asthma, allergy, allergic rhinitis, allergic airway inflammation, atopic dermatitis (AD), chronic obstructive pulmonary disease (COPD), Irritable bowel syndrome (IBS), arthritis, psoriasis, eosinophilic esophagitis, eosinophilic pneumonia, eosinophilic psoriasis, hypereosinophilic syndrome, and Eosinophilic Granulomatosis with Polyangiitis (Churg-Strauss Syndrome).

In certain embodiments, the methods of the present invention use control values for the frequency of subsets and cell states. In example embodiments, the control values can be determined for control samples that represent different states of severity along a trajectory from least severe to most severe (e.g., NOA to FR to PR). As used herein, “cell subset” refers to cells that belong to a specific cell type, such as T cells, goblet cells, dendritic cells, but can be distinguished among the specific cell type by a specific cell state or expression of specific genes. For example, subsets of T cells can include proliferating T cells, subsets of NK cells can include cytotoxic NK cells, subsets of monocytes/macrophages can include specific monocytes/macrophages, subsets of dendritic cells can include plasmacytoid dendritic cells (pDCs) and subsets of epithelial cells can include metabolically-specialized epithelial cell subsets. For example, the present cell atlases provide for the frequency of cell subsets and cell states for each of NOA, FR and PR, but control values can also be determined using additional annotated samples. As used herein the frequency of cell subsets (i.e., comparison of the number of cells) may be determined by the frequency of a subset amongst total cells or the frequency of a subset amongst its own cell type (e.g., T cell/Natural Killer/Innate lymphoid cell (T/NK/ILC), myeloid and/or epithelial cell subsets; or individual cell types within T cell/Natural Killer/Innate lymphoid cell (T/NK/ILC), myeloid and/or epithelial cell subsets). Applicants determined that the composition of cell types does not significantly differ between CD and FG samples. Thus, a change in frequency of a subset of the cell types in a sample can be detected by comparing the number of cells of a subset to the total of all cells or the total of all cells of the cell type. In example embodiments, the frequency of a subset of a specific cell type is compared to the total of the specific cell type. The determined frequency can then be compared to control values to determine risk for severity and treatment groups.

Cells such as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”. Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells. In regards to frequency, a cell subset may be present or not present. In certain embodiments, a cell subset may be 5, 10, 20, 30, 40, 50, 60, 70, 80 or 90% more frequent in a parent cell population as compared to a control level.

A Method of Stratifying Subjects Suffering from IBD

In one example embodiment, a method for stratifying subjects suffering from IBD into risk groups comprises detecting in a sample obtained from a subject the frequency of one or more T cell/Natural Killer/Innate Lymphoid cell (T/NK/ILC), myeloid and/or epithelial cell subsets selected from Table 1, and determining if the subject is in a well-controlled without anti-TNF-blockade (NOA) risk group, an anti-TNF-blockade full responder (FR) risk group, or anti-TNF-blockade partial responder (PR) risk group by comparing the frequency of the detected cell subsets to a control frequency for the subject along a trajectory of disease severity from NOA, to FR, to PR. Table 10 provides for frequencies of each subset in each pediCD patient.

Table 1 provides for cell subset specific gene markers in the pediCD atlas. Table 1A provides for subset specific markers for all subsets identified in the pediCD atlas. The genes with the lowest adjusted p value are shown first for each subset. As discussed herein, when the adj. p-value=0, expression within-cluster >40% of cells are positive for the gene and in other cells <6% of cells are positive for the gene. In one example embodiment, 1, 2, 3, 4, 5, 6, 7, 8, 9 or 10 or more genes are detected. In another example embodiment, detecting 2 or more of the subset markers increases the probability of detecting a cell subset. Table 1B provides for subset specific markers with a higher adjusted p value cutoff for subsets that are shifted in frequency between NOA, FR and PR.

In one example embodiment, the cell subsets have higher expression of one or more principle components (PC) determined using dimension reduction (see, e.g., Shalek, A. K. et al. Single-cell transcriptomics reveals bimodality in expression and splicing in immune cells. Nature 498, 236-240, doi:10.1038/naturel2172 (2013)). Cell subsets can be identified as clusters of cells using any dimension reduction method (see, e.g., Becht et al., Evaluation of UMAP as an alternative to t-SNE for single-cell data, bioRxiv 298430; doi.org/10.1101/298430; Becht et al., 2019, Dimensionality reduction for visualizing single-cell data using UMAP, Nature Biotechnology volume 37, pages 38-44; and Moon et al., PHATE: A Dimensionality Reduction Method for Visualizing Trajectory Structures in High-Dimensional Biological Data, bioRxiv 120378; doi: doi.org/10.1101/120378). Cell subsets or cell states can also be referred to by a cluster name. Table 3 shows PC loadings for the cell subsets in the pediCD atlas. Table 11 shows PCA Loadings for the joint Epithelial, Myeloid, T/NK/ILC vectors. In one example embodiment, cell subsets that are the top negative loadings of PC2 are most predictive of NOA, FR and PR. In certain embodiments, top cell subsets for the negative loadings of PC2 include one or more of cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, Mac.DC.CXCL10.CLEC4E, NK.GNLY.FCER1G, T.MKI67.IL22, NK.GNLY.IFNG, EC.OLFM4.MT.ND2, NK.GNLY.GZMB, Mono.Mac.CXCL10.CXCL11, Mono.FCN1.S100A4, T.CARD16.GB2, Mono.CXCL10.TNF, and NK.MKI67.GZMA. In one example embodiment, marker genes are detected for the top negative loadings for PC2. In certain embodiments, the subsets detected include one or more of cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, NK.GNLY.FCER1G, T.MKI67.IL22, NK.GNLY.IFNG, NK.GNLY.GZMB, Mono.FCN1.S100A4 and NK.MKI67.GZMA. In one example embodiment, the subsets detected include one or more of Goblet.RETNLB.ITLN1, Mac.CXCL3.APOC1, EC.NUPR1.LCN2, Mono.Mac.CXCL10.FCN1, NK.GNLY.FCER1G, Mono.FCN1.S100A4, and NK.MKI67.GZMA In one example embodiment, the subsets detected include one or more of cDC2.CD1C.AREG, T.MAF.CTLA4, T.CCL20.RORA, Mac.C1QB.CD14, Mono.CXCL3.FCN1, pDC.IRF7.IL3RA, T.GNLY.CSF2, Mono.Mac.CXCL10.FCN1, T.MKI67.FOXP3, T.MKI67.IFNG, NK.GNLY.FCER1G, NK.GNLY.IFNG, NK.GNLY.GZMB, and NK.MKI67.GZMA. In one example embodiment, the subsets detected include one or more of Mono.Mac.CXCL10.FCN1, NK.GNLY.FCER1G, and NK.MKI67.GZMA.

In one example embodiment, one or more cell subsets are detected that have a shift in frequency in NOA as compared to FR and PR. In one example embodiment, an increase in frequency of CD.NK.MKI67.GZMA and CD.T.MKI67.IL22 indicates FR or PR and a decreased frequency indicates NOA. In one example embodiment, a decrease in frequency of CD.Fibro.CCL19.IRF7 and CD.EC.SLC28A2.GSTA2 indicates FR or PR and an increased frequency indicates NOA.

In one example embodiment, one or more cell subsets are detected that have a shift in frequency in NOA as compared to PR. In one example embodiment, an increase in frequency of CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, and CD.Goblet.TFF1.TPSG1 indicates PR and a decreased frequency indicates NOA. In certain embodiments, a decrease in frequency of CD.T.LAG3.BATF, CD.T.IFI44L.PTGER4, and CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15 indicates PR and an increased frequency indicates NOA.

In one example embodiment, one or more cell subsets are detected that have a shift in frequency in NOA as compared to FR. In one example embodiment, a decrease in frequency of CD.EpithStem.LINC00176.RPS4Y1, CD.MCell.CSRP2.SPIB, CD.EC.FABP6.PLCG2, and CD.EC.FABP1.ADIRF indicates FR and an increased frequency indicates NOA.

In certain embodiments, one or more cell subsets are detected that have a shift in frequency in FR as compared to PR. In certain embodiments, an increase in frequency of CD.B/DZ.HIST1H1B.MKI67 indicates PR and a decreased frequency indicates FR.

In one example embodiment, cell subsets identified in FGID are detected. Table 4 provides for subset specific markers for each subset.

In another example embodiment, a method for stratifying subjects suffering from IBD into risk groups comprises detecting in a sample obtained from a subject one or more signature genes or a gene signature. Applicants have identified specific cell states, gene signatures, that are shifted along a trajectory of disease severity. Thus, detecting cell states can be used for diagnostic and therapeutic methods. In particular, the cell states are shifted between anti-TNF-blockade full responder (FR) and anti-TNF-blockade partial responder (PR) subjects. In one example embodiment, one or more differentially expressed genes are detected (Table 2). In one example embodiment, the one or more genes are detected in a specific cell subset. In one example embodiment, cell subset specific markers are used to determine a subset and one or more differentially expressed genes in that subset are detected in combination. Thus, one or more markers can be used to identify the cell subset and differentially genes can be detected in only that subset. In one example embodiment, genes differentially expressed between FR and PR are selected from Table 2A, 2B or 2C. Table 2A shows the top differentially expressed genes in each subset. Table 2B shows genes differentially expressed in the cell subsets having the most differentially expressed genes. In certain embodiments, APOA1, FABP6, NACA, APOA4, TPT1, SPINK4, MIF, IFITM1, HOPX, and HOPX are increased in FR relative to PR, and TNFRSF11B, TFPI2, SERPINE2, GSN, COL1A1, HIF1A, COL1A2, CTNNB1, CCL11, EMILIN1, CEBPB, SLC16A4, HTRA3, CMC1, AREG, COL4A1, SKIL, KLRC1, PTGER4, BRI3, APOE, BDKRB1, TXN, GPR65, NKG7, SAMHD1, CLEC12A, STAT1, PFN1, and TAX1BP1 are increased in PR relative to FR. Table 2C shows all of the differentially expressed genes in the two subsets with the most differentially expressed genes. In certain embodiments, the cell state is a gene program comprising one or more up and down regulated genes. In example embodiments, one or more genes of cell states associated with disease severity and treatment outcomes are detected. In example embodiments, the disease severity gene signature includes one or more of the top 92 markers of the 25 cell states associated with disease severity and treatment outcomes (Table 14). In example embodiments, one or more of TNFAIP6, GZMB, S100A8, CSF2, CLEC4E, S100A9, IL1RN, FCGR1A, CLIC3, CD14, PLA2G7, FAM26F, IL3RA, NKG7, IL32, CCL3, OLR1, LILRA4, APOC1 and MYBL2 are detected to predict anti-TNF therapy outcome in newly diagnosed patients. In example embodiments, the one or more genes are detected in bulk samples or in single cells.

Clusters (subsets) and gene programs as described herein can also be described as a metagene. As used herein a “metagene” refers to a pattern or aggregate of gene expression and not an actual gene. Each metagene may represent a collection or aggregate of genes behaving in a functionally correlated fashion within the genome. The metagene can be increased if the pattern is increased. As used herein the term “gene program” or “program” can be used interchangeably with “cell state”, “biological program”, “expression program”, “transcriptional program”, “expression profile”, “signature”, “gene signature” or “expression program” and may refer to a set of genes that share a role in a biological function (e.g., an inflammatory program, cell differentiation program, proliferation program). Biological programs can include a pattern of gene expression that result in a corresponding physiological event or phenotypic trait (e.g., inflammation). Biological programs can include up to several hundred genes that are expressed in a spatially and temporally controlled fashion. Expression of individual genes can be shared between biological programs. Expression of individual genes can be shared among different single cell subtypes; however, expression of a biological program may be cell subtype specific or temporally specific (e.g., the biological program is expressed in a cell subtype at a specific time). Multiple biological programs may include the same gene, reflecting the gene's roles in different processes. Expression of a biological program may be regulated by a master switch, such as a nuclear receptor or transcription factor.

As used herein a “signature” or “gene program” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of immune cells that are linked to particular pathological condition (e.g., inflammation), or linked to a particular outcome or progression of the disease (e.g., autoimmunity), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

As used herein, all gene name symbols refer to the gene as commonly known in the art. The examples described herein that refer to the human gene names are to be understood to also encompasses mouse genes, as well as genes in any other organism (e.g., homologous, orthologous genes). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). The signature as described herein may encompass any of the genes described herein.

In certain embodiments, detecting cell subset markers or differentially expressed genes can be used to determine a treatment for a subject suffering from a disease or stratify a subject. The invention provides biomarkers (e.g., phenotype specific or cell subtype) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations who would benefit or not benefit from anti-TNF blockade based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the cells in the subject compared to a control subject having normal status or not having a disease indicates response to treatment. Hence, the methods may rely on comparing the quantity of cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or 1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Example Methods of Detection

In one embodiment, the signature genes, biomarkers, and/or cells may be detected by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) (Chen et al., Spatially resolved, highly multiplexed RNA profiling in single cells. Science, 2015, 348:aaa6090; and Xia et al., Multiplexed detection of RNA using MERFISH and branched DNA amplification. Sci Rep. 2019 May 22; 9(1):7721. doi: 10.1038/s41598-019-43943-8), ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample (e.g., a biopsy).

The present invention also may comprise a kit with a detection reagent that binds to one or more biomarkers or can be used to detect one or more biomarkers.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Sequencing

In certain embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8) Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol Biol. 2009; 513:19-39); and Morozova et al (Genomics. 2008 92:255-64), which are incorporated by reference for the general descriptions of the methods and the particular steps of the methods, including all starting products, reagents, and final products for each of the steps. In example embodiments, sequencing includes bulk RNA sequencing (R-NA-seq)

### Single Cell Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Treatment Selection

In one example embodiment, a method of treatment comprises stratifying subjects suffering from IBD into risk groups as described herein and further comprising selecting a treatment, wherein if the subject is in the NOA group, then treating the subject with a treatment that does not comprise anti-TNF-blockade; if the subject is in the FR group, then treating the subject with a treatment comprising anti-TNF-blockade; and if the subject is in the PR group, then treating the subject with a treatment comprising anti-TNF-blockade and/or an additional treatment. In one example embodiment, the method for stratifying subjects suffering from IBD into risk groups comprises detecting in a sample obtained from a subject the frequency of one or more T cell/Natural Killer/Innate Lymphoid cell (T/NK/ILC), myeloid and/or epithelial cell subsets selected from Table 1, and determining if the subject is in a well-controlled without anti-TNF-blockade (NOA) risk group, an anti-TNF-blockade full responder (FR) risk group, or anti-TNF-blockade partial responder (PR) risk group by comparing the frequency of the detected cell subsets to a control frequency for the subject along a trajectory of disease severity from NOA, to FR, to PR. In one example embodiment, the method for stratifying subjects suffering from IBD into risk groups comprises detecting in a sample obtained from a subject one or more signature genes or a gene signature selected from Table 2 or Table 14.

There is currently no cure for Crohn's disease, and there is no single treatment that works for all subjects. In certain embodiments, the methods of the present invention are used to select any treatment within the current standard of care and provide for less toxicity and improved treatment. In preferred embodiments, the treatment selected is anti-TNF blockade. The term “standard of care” as used herein refers to the current treatment that is accepted by medical experts as a proper treatment for a certain type of disease and that is widely used by healthcare professionals. Standard of care is also called best practice, standard medical care, and standard therapy. In example embodiments, the present invention provides improved treatment selection, for example, PCDAI (Pediatric Crohn's Disease Activity Index) (see, e.g., Zubin G, Peter L. Predicting Endoscopic Crohn's Disease Activity Before and After Induction Therapy in Children: A Comprehensive Assessment of PCDAI, CRP, and Fecal Calprotectin. Inflamm Bowel Dis. 2015; 21(6):1386-1391).

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

In certain embodiments, the therapeutic agents are administered in an effective amount or therapeutically effective amount. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In certain embodiments, IBD is treated by selecting subject who will benefit from anti-TNF blockade. Inflammatory bowel disease (IBD) is a chronic disabling inflammatory process that affects mainly the gastrointestinal tract and may present associated extraintestinal manifestations (see, e.g., Catalan-Serra I, Brenna Ø. Immunotherapy in inflammatory bowel disease: Novel and emerging treatments. Hum Vaccin Immunother. 2018; 14(11):2597-2611). IBD includes both ulcerative colitis (UC) and Crohn's disease (CD). Id. Current pharmacological treatments used in clinical practice like thiopurines or anti-TNF are effective but can produce significant side effects and their efficacy may diminish over time. Id. The current treatment of IBD includes mesalazine (oral and rectal formulations), glucocorticoids (conventional and other forms like budesonide or beclomethasone), antibiotics (typically ciprofloxacine and metronidazole), immunosuppressants (mostly azathioprine/6-mercaptopurine or methotrexate) and anti-TNF agents (infliximab, adalimumab, certolizumab pegol and golimumab). Recently, the anti-integrin antibody vedolizumab and the antibody against IL-12/23 ustekinumab have been approved for IBD. Id. Corticosteroids may be used for short-term (three to four months) symptom improvement and to induce remission. Corticosteroids may also be used in combination with an immune system suppressor. Azathioprine (Azasan, Imuran) and mercaptopurine (Purinethol, Purixan) are the most widely used immunosuppressants for treatment of inflammatory bowel disease. Taking them requires follow up to look for side effects, such as a lowered resistance to infection and inflammation of the liver. Methotrexate (Trexall) is sometimes used for people with Crohn's disease who don't respond well to other medications.

In certain embodiments, selecting subjects that are responsive can be used to avoid producing significant side effects in subjects that will not benefit from the treatment. In certain embodiments, an alternative treatment is administered to non-responsive subjects such that side effects are diminished. In certain embodiments, a drug is administered to shift a subject to be responsive.

### TNF Inhibitors

The present invention also contemplates use of tumor necrosis factor (TNF) inhibitors for treatment (e.g., anti-TNF blockade). In certain embodiments, the invention described herein is related to a method of treatment in which one or more TNF inhibitors are administered to a patient in need thereof, treatment which may be determined in whole or in part by the systems and methodologies described herein. In one embodiment, TNF-α inhibitor antibodies, or antigen binding fragments thereof, are contemplated for use. In an aspect, the TNF inhibitor is an immunosuppressive medication. In an embodiment, the TNF inhibitor is a monoclonal antibody. In particular embodiments, the TNF inhibitor binds to soluble forms of TNF-alpha, the transmembrane form of TNF-alpha, or both forms of TNF-alpha. In one example embodiment, the TNF inhibitor is adalimumab or a biosimilar thereof. The TNF inhibitor may comprise a chimeric antibody, such as infliximab or a biosimilar thereof, which comprises the TNF alpha trimer, a variable murine binding site for TNF-alpha and an Fc constant region. In an embodiment, the anti-TNF antibody is certolizumab pegol or golimumab or a biosimilar thereof. In an aspect, the inhibitor may comprise enhancing soluble TNF receptor 2, a receptor that binds to TNF-alpha by either delivery of a fusion protein or by the upregulation of TNF receptor 2 expression. Thus, in an embodiment, the TNF inhibitor is etanercept, a circulating TNF receptor-IgG fusion protein that binds to TNF-alpha. Administration of treatments etanercept, adalimumab, certolizumab and golimumab may be subcutaneous. Administration of infliximab and golimumab may be intravenous.

Small molecules such as thalidomide, lenalidomide and pomalidomide may also be used for treatment. Additionally, oral pentoxifylline or bupropion have also been used as TNF-alpha inhibitor treatment. See, e.g. Brustolim D, Ribeiro-dos-Santos R, Kast R E, Altschuler E L, Soares M B,). Int. Immunopharmacol. 6 (6): 903-7. doi:10.1016/j.intimp.2005.12.007 (June 2006) (buprioprion lowers production of TNF-alpha in mice. In an aspect, 5-HT_{2A} receptor agonists such as (R)-DOI, N,N-Dimethyltryptamine, paliperidone, APD791, YKP-1358, lurasidone, lisuride, methysergide, lorcaserin and other agonists known in the art may be utilized for treatment. See, eg. Yu et al., “Serotonin 5-Hydroxytryptamine_{2A} Receptor Activation Suppresses Tumor Necrosis Factor-α-Induced Inflammation with Extraordinary Potency,” J. Pharm and Exp Ther. Nov. 2008, 327(2) 316-323; doi: 10.1124/jpet.108.143461. Additionally, activation of HT2A receptors via genome editing may also be utilized for inhibition of TNF-alpha.

TNFR1 and/or TNFR2 receptors of TNF-alpha may be targeted for inhibition of TNF-alpha. In an example embodiment, CRISPR based systems may be used for the repression or activation of inflammatory cytokine cell receptor TNFR1 and/or anti-inflammatory and antiapoptotic interactions at TNFR2 receptors of TNF-alpha. See, Farhang et al., Tissue Eng Part A. 2017 Aug. 1; 23(15-16): 738-749, doi: 10.1089/ten.tea.2016.0441. Inhibition of the activation of the extracellular signal-regulated kinase may also be a target for RNAi or CRISPR related treatments or small molecule administration. In one embodiment, gliovirin, an epipolythiodiketopiperazine that suppresses TNF-alpha synthesis by inhibiting the activation of extracellular signal-regulated kinase (ERK) may be utilized. See, Rether et al., Biol Chem. 2007 Jun; 388(6):627-37 doi: 10.1515/BC.2007.066. Knockdown of TNF-alpha by DNAzyme gold nanoparticles is also contemplated for use as treatment, with local injection being one approach for treatment with DNA-zyme-conjugated particles. See, e.g. Somasuntharam et al., Biomaterials. 2016 March; 83:12-22. doi: 10.1016/j.biomaterials.2015.12.02.

### Additional Treatments

In example embodiments, subjects that are not fully responsive to TNF inhibitors are treated with additional treatments specific to those subjects. In example embodiments, the additional treatments target cell subsets enriched in frequency in subjects that are partial responders. In example embodiments, the additional treatments target genes or pathways differentially expressed in cell subsets in subjects that are partial responders. In example embodiments, the additional treatments are administered in combination with TNF inhibitors. In example embodiments, additional treatments include CD40L-blocking antibodies, IL-22 agonists, agents blocking inflammatory cytokines, such as IL-1, targeted anti-proliferation agents, and anti-GM-CSF antibodies (Betts et al., 2017; Lindemans et al., 2015; Miura et al., 2021; Ramanujam et al., 2020; Sootome et al., 2020; Ai et al., 2021; Aschenbrenner et al., 2021; Castro-Dopico et al., 2020; Mehta et al., 2020; Mitsialis et al., 2020; Muro and Mrowiec, 2015). In other example embodiments, any standard of care treatment discussed above can be used as an additional treatment. In example embodiments, one or more of the additional treatments are administered in combination with a standard treatment. The combinations may provide for enhanced or otherwise previously unknown activity in the treatment of disease. In certain embodiments, targeting the combination may require less of the standard agent as compared to the current standard of care and provide for less toxicity and improved treatment.

Non-limiting examples of CD40L inhibitors include toralizumab/IDEC-131 (see, e.g., Fadul C E, Mao-Draayer Y, Ryan K A, et al. Safety and Immune Effects of Blocking CD40 Ligand in Multiple Sclerosis. Neurol Neuroimmunol Neuroinflamm. 2021; 8(6):e1096) and CDP7657 (see, e.g., Shock A, Burkly L, Wakefield I, et al. CDP7657, an anti-CD40L antibody lacking an Fc domain, inhibits CD40L-dependent immune responses without thrombotic complications: an in vivo study. Arthritis Res Ther. 2015; 17(1):234).

Non-limiting examples of IL-22 agonists include an IL-22 polypeptide, an IL-22 Fc fusion protein, an IL-22 agonist, an IL-19 polypeptide, an IL-19 Fc fusion protein, an IL-19 agonist, an IL-20 polypeptide, an IL-20 Fc fusion protein, an IL-20 agonist, an IL-24 polypeptide, an IL-24 Fc fusion protein, an IL-24 agonist, an IL-26 polypeptide, an IL-26 Fc fusion protein, an IL-26 agonist, an IL-22R1, an antibody that binds IL-22BP and blocks or inhibits binding of IL-22BP to IL-22, and TLR7 agonists (see, e.g., U.S. patent Ser. No. 11/155,591B2; US Patent Application US20210338778A1; Wang Q, Kim S Y, Matsushita H, et al. Oral administration of PEGylated TLR7 ligand ameliorates alcohol-associated liver disease via the induction of IL-22. Proc Natl Acad Sci USA. 2021; 118(1):e2020868118).

Non-limiting examples of anti-GM-CSF antibodies include Gimsilumab, lenzilumab, namilumab, and otilimab, which target GM-CSF directly, neutralizing the biological function of GM-CSF by blocking the interaction of GM-CSF with its cell surface receptor (see, e.g., Mehta P, Porter J C, Manson J J, et al. Therapeutic blockade of granulocyte macrophage colony-stimulating factor in COVID-19-associated hyperinflammation: challenges and opportunities. Lancet Respir Med. 2020; 8(8):822-830; Lang F M, Lee K M, Teijaro J R, Becher B, Hamilton J A. GM-CSF-based treatments in COVID-19: reconciling opposing therapeutic approaches. Nat Rev Immunol. 2020; 20(8):507-514; and Temesgen Z, Assi M, Shweta F N U, et al. GM-CSF Neutralization with lenzilumab in severe COVID-19 pneumonia: a case-cohort study. Mayo Clin Proc. 2020; 95(11):2382-2394). Non-limiting examples of anti-GM-CSF antibodies also include Mavrilimumab, which targets the alpha subunit of the GM-CSF receptor, blocking intracellular signaling of GM-CSF (see, e.g., Lang F M, Lee K M, Teijaro J R, Becher B, Hamilton J A. GM-CSF-based treatments in COVID-19: reconciling opposing therapeutic approaches. Nat Rev Immunol. 2020; 20(8):507-514; and Burmester G R, Feist E, Sleeman M A, Wang B, White B, Magrini F. Mavrilimumab, a human monoclonal antibody targeting GM-CSF receptor-alpha, in subjects with rheumatoid arthritis: a randomised, double-blind, placebo-controlled, Phase I, first-in-human study. Ann Rheum Dis. 2011; 70(9):1542-1549).

### Screening Methods

### Identifying Novel and Improved Treatment

In certain embodiments, the cell subset frequency and/or differential cell states can be detected for screening novel therapeutic agents. In certain embodiments, the present invention can be used to identify improved treatments by monitoring the identified cell states in a subject undergoing an experimental treatment. In certain embodiments, an animal model is used to detect shifts in the identified cell states to identify agents capable of shifting a subject from a PR to FR or NOA.

In certain embodiments, the cell states identified herein are detected in a mouse model of an inflammatory disease. Exemplary IBD mouse models include those which are chemically-induced, those which are achieved by adoptive transfer of T cell subsets, and those that develop spontaneously in genetically modified mice, such as Acute and chronic dextran sulfate sodium (DSS)-induced colitis mouse models, poly I:C-induced intestinal inflammation model, trinitrobenzene sulfonic acid (TNBS)-induced colitis mouse model, Adoptive transfer of CD4+CD45RBhigh T cells, IL-10 KO mice (see, e.g., Boismenu R, Chen Y. Insights from mouse models of colitis. J Leukoc Biol. 2000 March; 67(3):267-78, Table 2).

In certain embodiments, candidate agents are screened. The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the present invention provides for gene signature screening to identify agents that shift expression of the gene targets described herein (e.g., cell subset markers and differentially expressed genes). The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to identify small molecules capable of modulating a signature or biological program of the present invention in silico.

Further embodiments are illustrated in the following Examples which are given for illustrative purposes only and are not intended to limit the scope of the invention.

### EXAMPLES

### Example 1—A Treatment-Naïve Single-Cell Atlas from Inflammatory Disease Conditions

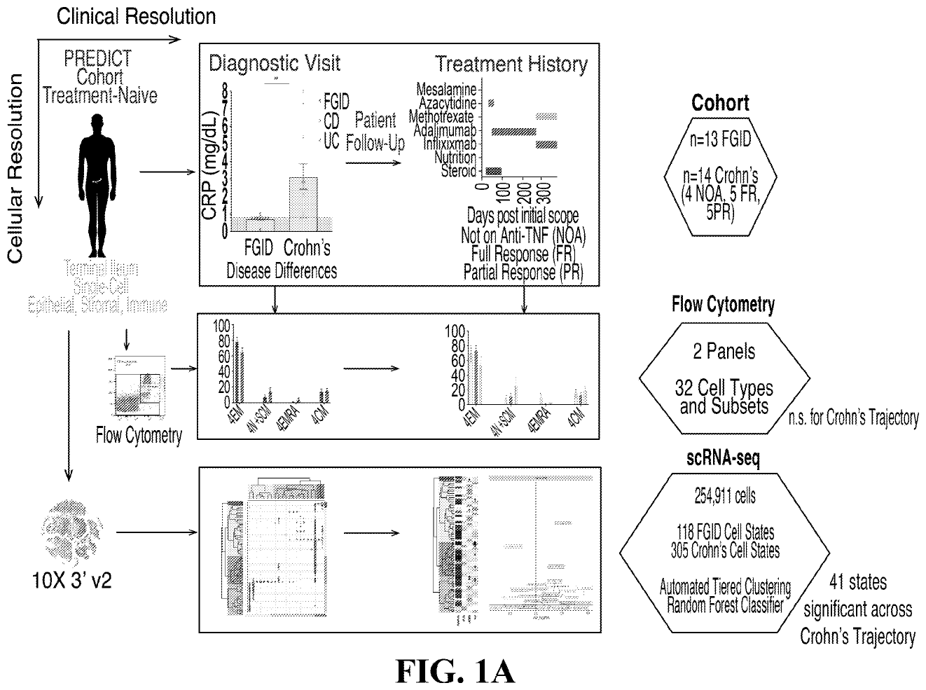

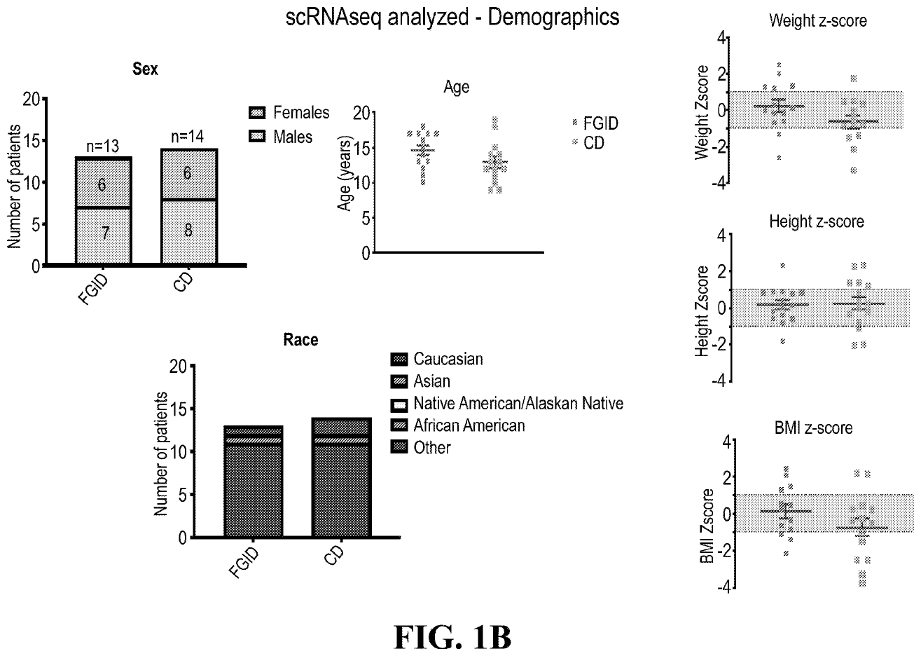

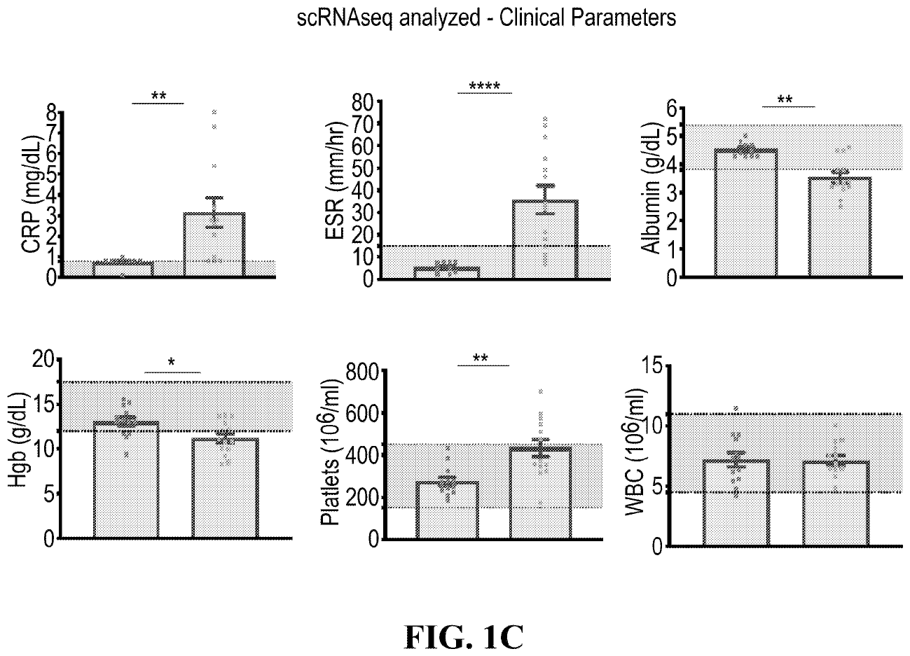

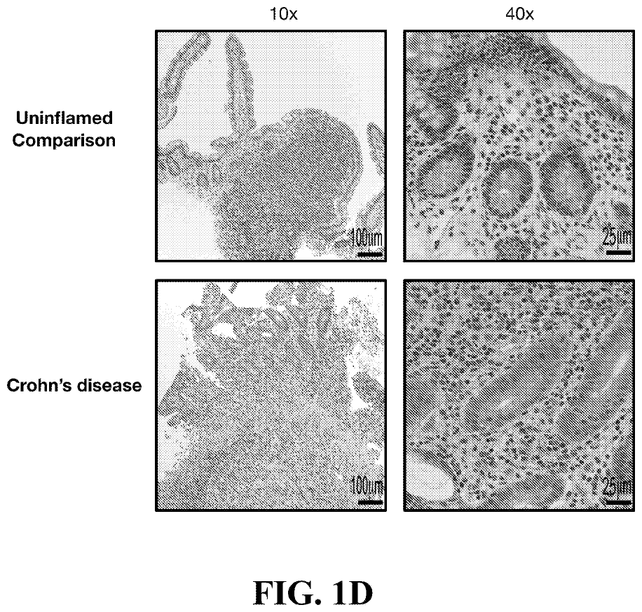

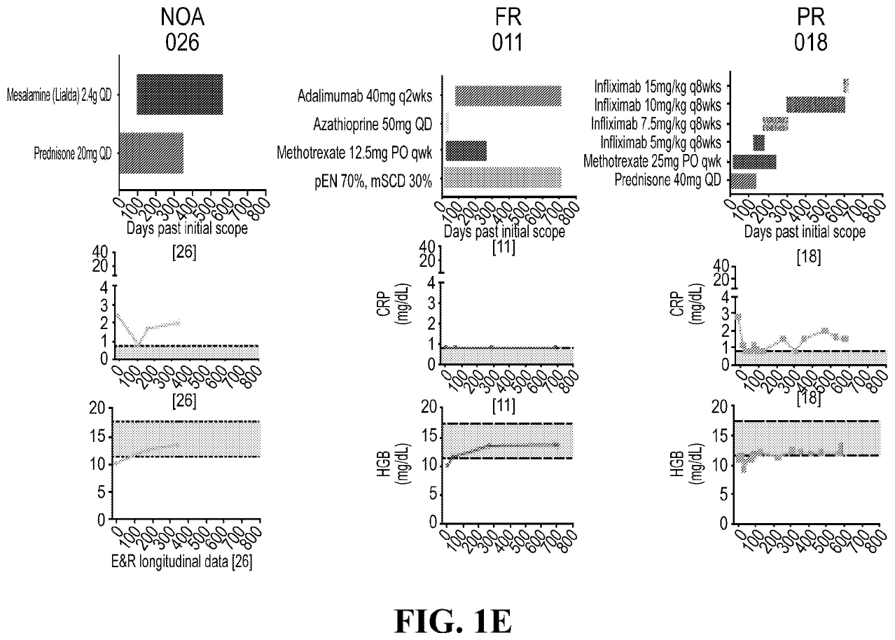

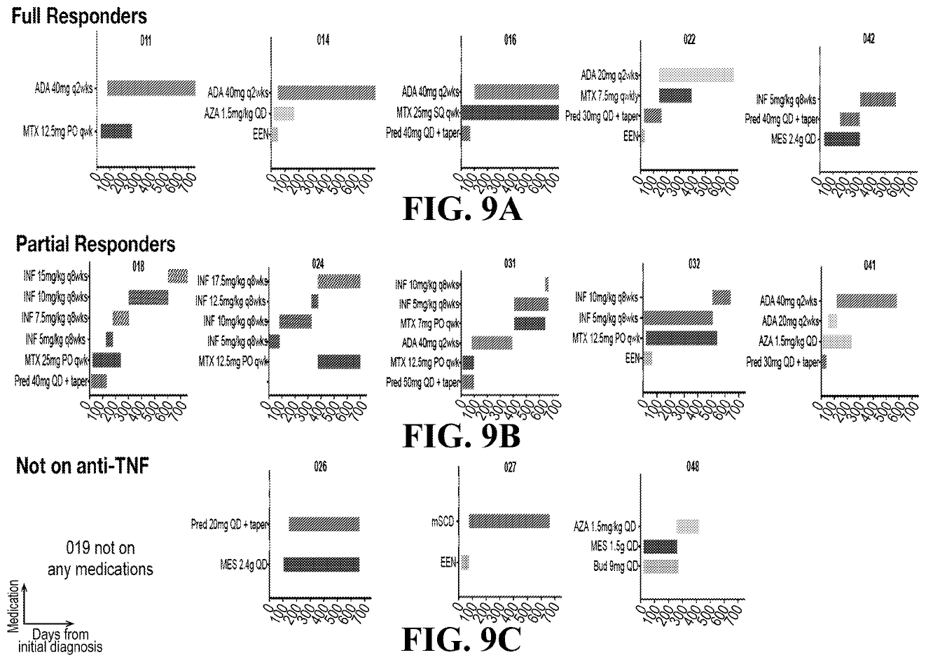

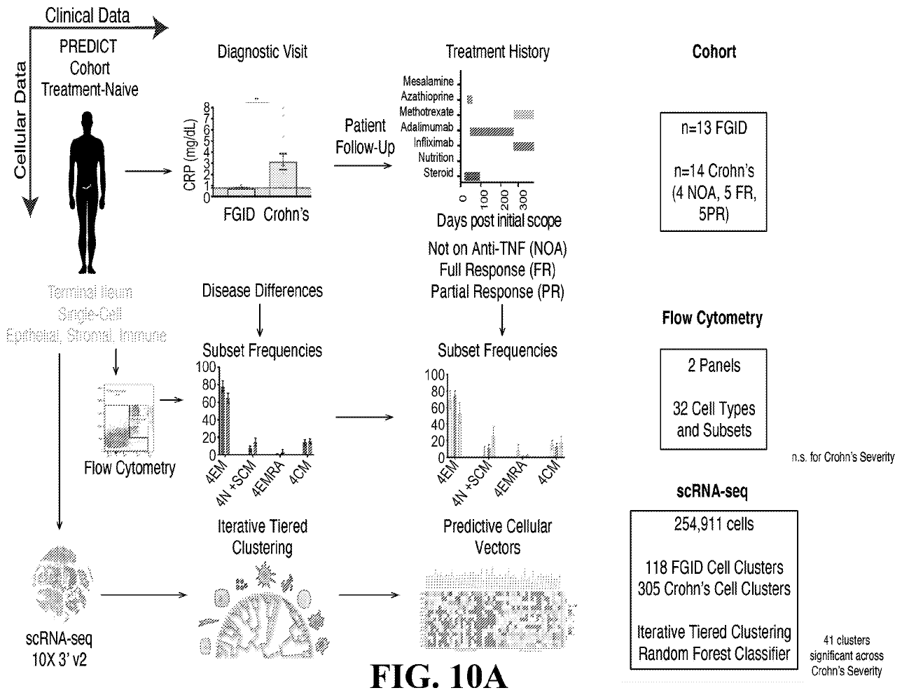

To Applicants knowledge, all present scRNA-seq comprehensive atlases of inflammatory disease conditions consist of patients being treated with a variety of agents, and for which the biopsies included in these studies often reflect a partial treatment-refractory state to combinations of antibiotics, 5-ASA, corticosteroids, and anti-TNF mAbs. A treatment-naïve single-cell atlas in any inflammatory disease condition has yet to be reported. In order to address this unmet need and generate a comprehensive cellular atlas from treatment-naïve pediCD compared to uninflamed age-matched controls, Applicants created the prospective PREDICT study (Clinicaltrials.gov #NCT03369353) to help identify, profile, and understand pediatric IBD and FGID. Here, Applicants present detailed diagnostic and treatment data from the first cohort of 27 patients enrolled on PREDICT, including 14 pediCD and 13 FGID patients, together with flow cytometric and scRNA-seq studies of the cellular composition of the terminal ileum (FIG. 1 and FIG. 9). Furthermore, through detailed, prospective clinical metadata and longitudinal follow-up, Applicants stratify the pediCD cohort by clinically-guided therapeutic decisions separating patients treated with anti-TNF mAbs versus those with biopsy-proven pediCD, but for whom clinical symptoms were sufficiently mild that the treating physician did not prescribe anti-TNF agents (this cohort is termed “Not On Anti-TNF” or “NOA”). Applicants were also able to separate patients treated with anti-TNF agents who achieved a full response (FR) versus a partial response (PR). Importantly, because PREDICT enrolled patients prior to their diagnostic endoscopy, Applicants were able to relate these clinical characteristics and outcomes to the patients' cell states at diagnosis. Applicants contextualize the findings in pediCD relative to 13 FGID patients which provides a symptomatic, age-matched, but non-inflammatory disease manifestation as a comparator group. Together Applicants present two cellular atlases for pediatric GI disease, consisting of 99,488 cells for FGID and 124,054 for pediCD, fully-annotate all cells, provide key gene-list resources for further studies, identify correspondence between them, and identify cell states and gene expression profiles associated with disease severity and treatment outcomes.

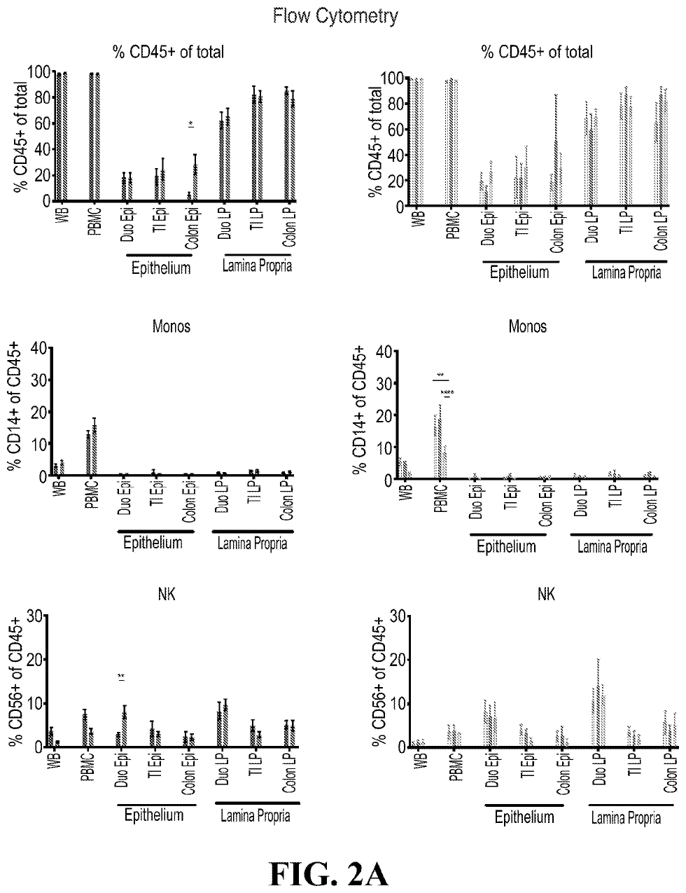

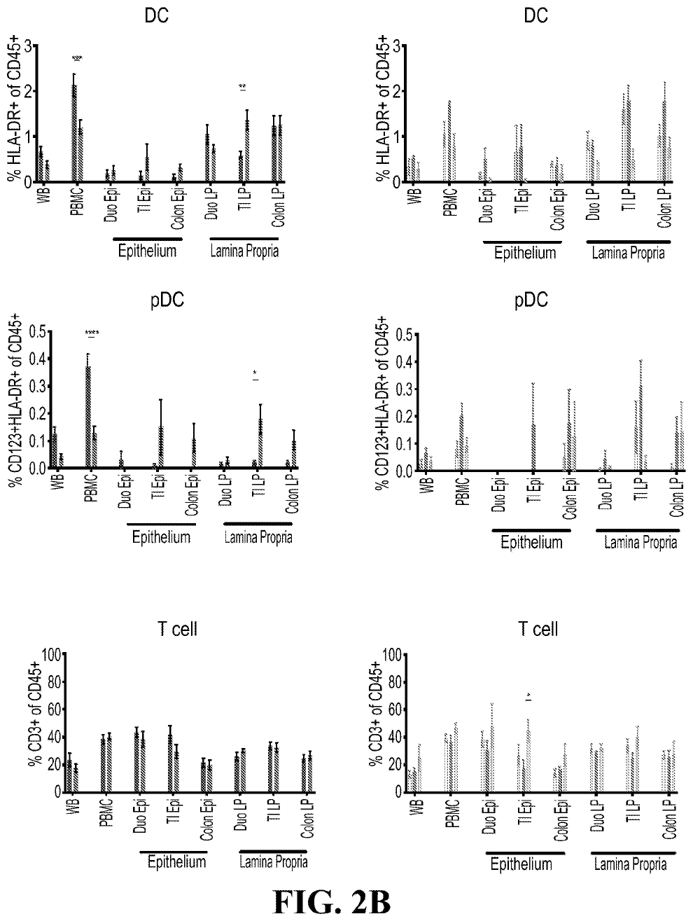

Applicants collected terminal ileum biopsies from 13 FGID patients and from 14 pediCD patients, and prepared single cell suspensions for flow cytometry and scRNA-seq. Biopsies from pediCD were from inflamed areas adjacent to active ulcerations. Biopsies from FGID were also taken. The epithelium was first separated from the lamina propria before enzymatic dissociation, and flow cytometric analysis was performed on the remaining viable single-cell fraction which recovered predominantly hematopoietic cells with some remnant epithelial cells (<20% of all cells), likely representing those in deeper crypt regions (FIG. 2). Applicants utilized two flow cytometry panels allowing for resolving the principal lymphoid (CD4 or CD8 T cells, NK cells, B cells, innate lymphoid cells, gD T cells, CD8aa+ IELs, plasmacytoid dendritic cells) and myeloid (monocytes, granulocytes, HLA-DR+ mononuclear phagocytes) cell subsets. From the 32 gates identifying cell lineages, types and subsets, only HLA-DR+ macrophages/DCs and plasmacytoid dendritic cells were significantly increased in pediCD relative to FGID (FIG. 2). Applicants also analyzed within pediCD, comparing the baseline samples of 4 NOA, 5 FR and 5 PR patients, and noted no significant differences between NOA and patients on anti-TNF, or between FRs and PRs to anti-TNF. Together, this suggests that despite the substantial endoscopic, histologic and clinical parameters that distinguish FGID and pediCD, the basic single-cell composition of the terminal ileum appears minimally altered in pediCD save for an increase in pDC and HLA-DR+ macrophages/dendritic cells.

In addition to flow cytometry, Applicants performed droplet-based scRNA-seq on cell suspensions from the 14 pediCD/13 FGID patient cohort using the 10× Genomics V2 3′ platform (FIG. 1). The analyzed cell suspensions were derived from lamina propria preparations, and the flow cytometry data suggested these would be composed primarily of CD45+ leukocytes, alongside a small fraction of epithelial cells and stromal/vascular cells. Deconstructing these tissues into their component cells provides for the ability to identify some of the corresponding cell types (e.g. T or B cell) and subsets (CD8aa+ IEL or CD4+ T cell) as Applicants identified in the flow cytometry data but importantly enabled Applicants to: 1. characterize these major cell types and subsets using a principled hierarchical heuristic without needing to pre-select markers, and 2. gain substantially enhanced resolution into the cell states (i.e. gene expression programs) within these types and subsets.

Following library preparation and sequencing, Applicants derived a unified cells-by-genes expression matrix from the 27 samples, containing digital gene expression values for all cells passing quality thresholds (n=254,911 cells). Applicants then performed dimensionality reduction and graph-based clustering, noting that despite no integration methods being used, FGID and CD were essentially indistinguishable from each other when visualized on a uniform manifold approximation and projection (UMAP) plot. Applicants recovered the following cell types from both patient groups: epithelial cells, T cells, B cells, plasma cells, glial cells, endothelial cells, myeloid cells, mast cells, fibroblasts, and a proliferating cluster. Applicants noted that the fractional composition amongst all cells of T cells, B cells, and myeloid cells was not significantly different between FGID and pediCD, similar to the flow cytometric data, and this was also the case for endothelial, epithelial, fibroblasts, glial, mast and plasma cells, which were not measured through flow cytometry. This provided validation and extension of the flow cytometry data that the broad cell type composition of FGID and pediCD is not significantly altered, despite highly distinct clinical diseases.

Applicants then systematically re-clustered each broad cell type, identifying increasing heterogeneity within each type. Given that Applicants detected changes in the frequency of HLA-DR+ macrophages/dendritic cells and pDCs by flow cytometry, Applicants initially focused on the myeloid cell type sub-clustering, containing dendritic cells, macrophages, monocytes, and pDCs. However, it soon became evident that this traditional clustering approach raised several challenges with identifying the boundaries of clusters, and whether a cluster composed primarily of pediCD cells represented a unique cell subset, or a cell state overlaid onto a core cell subset gene expression program (Methods). This would influence whether a comparison would primarily focus on differential expression testing or differential composition testing. It also raised the possibility that this joint clustering approach, informed by the inclusion of both FGID and pediCD cell types, subsets and states could muddle some of the unique biology of FGID and pediCD. This could lead to clusters, and correspondingly critical gene-reference lists for each cluster, that may not accurately represent that cell type, subset, or state, as the cluster is representative of a hybrid informed by cells from an FGID and pediCD intestine.

In order to approach this challenge from a more principled direction, Applicants made four key changes to the analytical workflow: 1. Applicants proceeded to analyze FGID and pediCD samples separately to define cell type, subset, and state clusters and markers, 2. implemented an automated iterative tiered clustering (ITC) approach to optimize the silhouette score at each tier of iterative sub-clustering and stop when a specific granularity is reached, 3. accounted for the diversity of patients which compose that cluster using Simpson's Index of Diversity, and 4. generated and optimized a Random Forest classifier to identify correspondence between the resultant FGID and pediCD atlases (Methods). Using this approach, each tier of analysis is typically under-clustered relative to traditional empirical analyses, but the automation proceeds through several more tiers (typically 6 to 7) until stop conditions (e.g. cell numbers and differentially expressed genes, see Methods) are met. Applicants then inspected all outputs (FGID and pediCD clusters) and provided descriptive cell cluster names independently for FGID and pediCD. Applicants also focused at this stage on flagging putative doublet clusters or clusters where the majority of differentially-expressed genes which triggered further clustering consist of known technical confounders in scRNA-seq data (e.g. mitochondrial, ribosomal, and spillover genes from cells with high secretory capacity) but did not remove them, as end users of this resource are likely to encounter these clusters and may be interested in their prospective identification.

Applicants then hierarchically clustered all end cell state clusters in order to generate the final dendrograms for FGID and pediCD, and performed 1 vs. rest within-cell-type differential expression to provide systematic names for cells based on their cell type classification and two genes (Methods). As several cell types contained readily identifiable and meaningful cell subsets, Applicants utilized curation of literature-based markers to provide further guidance within each cell type. For example, within Tier 1 T Cells Applicants could identify T cells, NK cells and ILCs, within Tier 1 Myeloid cells, Applicants could identify monocytes, cDC1, cDC2, macrophages and pDCs, within Tier 1 B cells germinal center, germinal center dark zone and light zone cells, and within Tier 1 Endothelial cells Applicants could identify arterioles, capillaries, lymphatics, mural cells and venules, and so forth for other cell types. To illustrate this process for one cluster, upon automated hierarchical tiered clustering of T cells, Applicants identified a cluster that was Tier 0: pediCD, Tier 1: T cells, Tier 2: cytotoxic, Tier 3: IEL_FCER1G_NKG7_TYROBP_CD160_AREG. Upon inspection of CD3 genes (CD247, CD3D, etc.), TCR genes (TRAC, TRBC1, etc.), and NK cell genes (NCAM1, NCR1), it became readily apparent these cells were NK cells, and 1 vs. rest within-cell-type differential expression identified CCL3 and CD160 as two genes significantly enriched in this cluster (adj. p-value=0, expression within-cluster>40% cells positive and in other Tier 1 T cells<6%). This resulted in a final name for this cluster of CD.NK.CCL3.CD160. Applicants repeated this process for all FGID (183 end clusters) and pediCD (426 end clusters) within Tier 1 B cell, Endothelial, Epithelial, Fibroblast, Plasma Cell, Myeloid Cell, Mast Cell, and T Cell identified clusters, and provide systematically generated names for all, as well as 1 vs. rest within-cell type gene lists (Table 1 and Table 4).

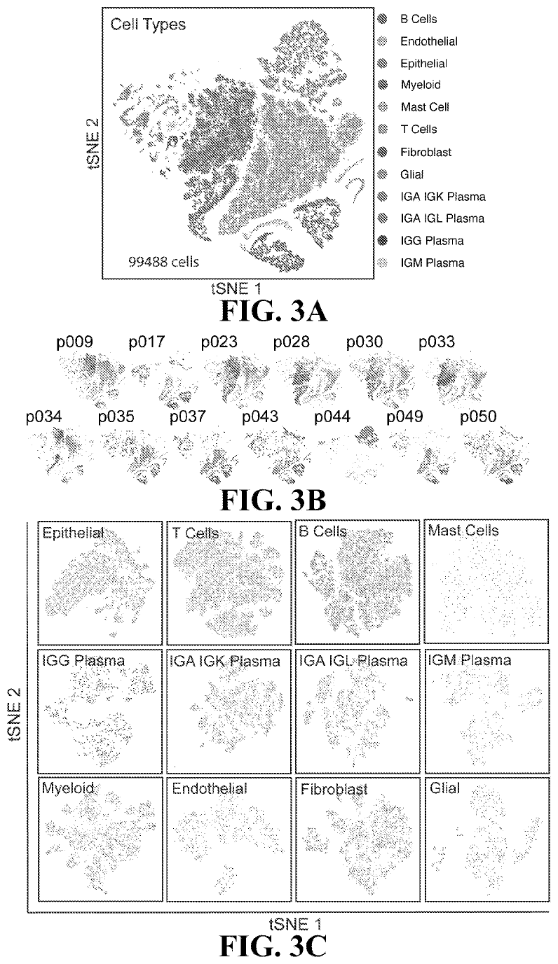

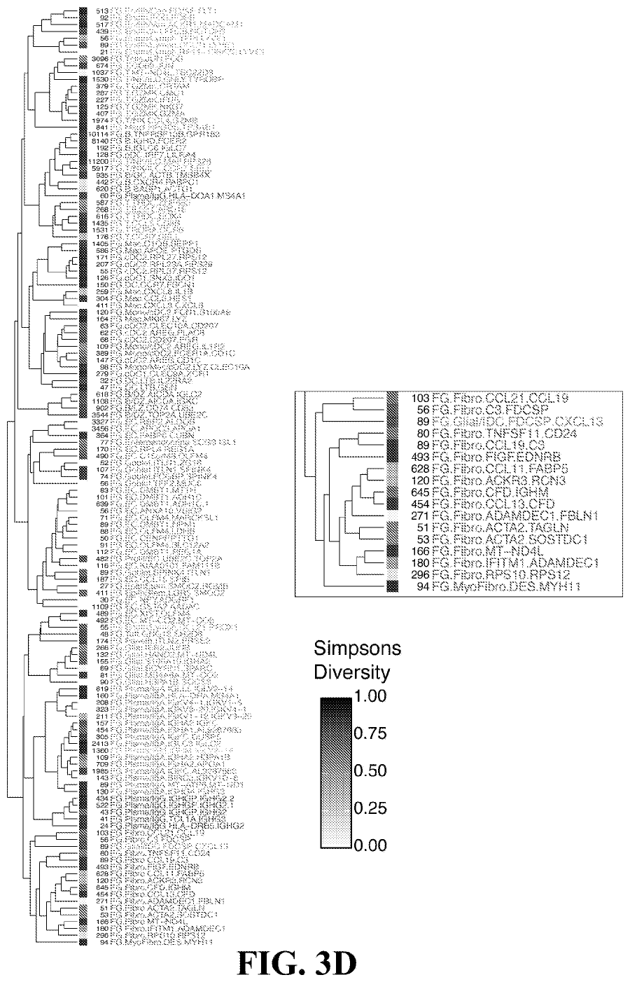

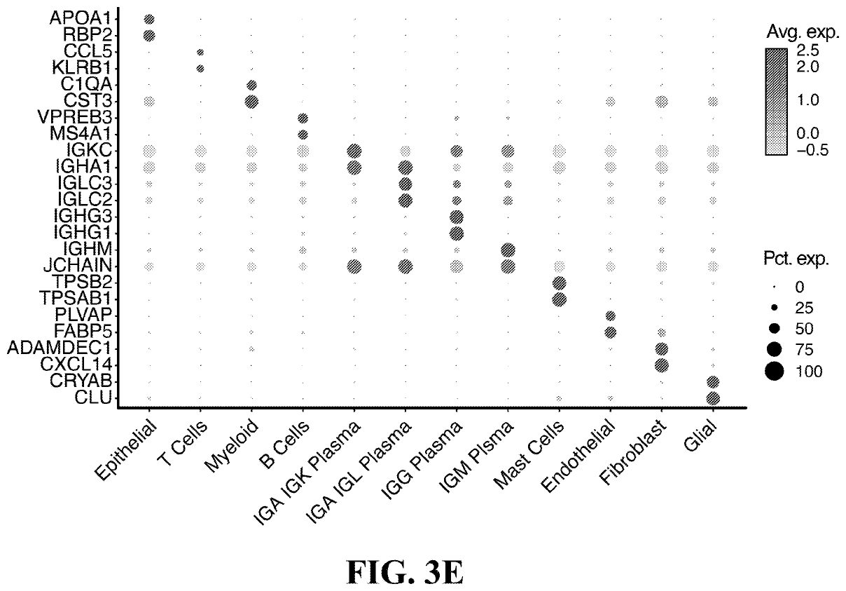

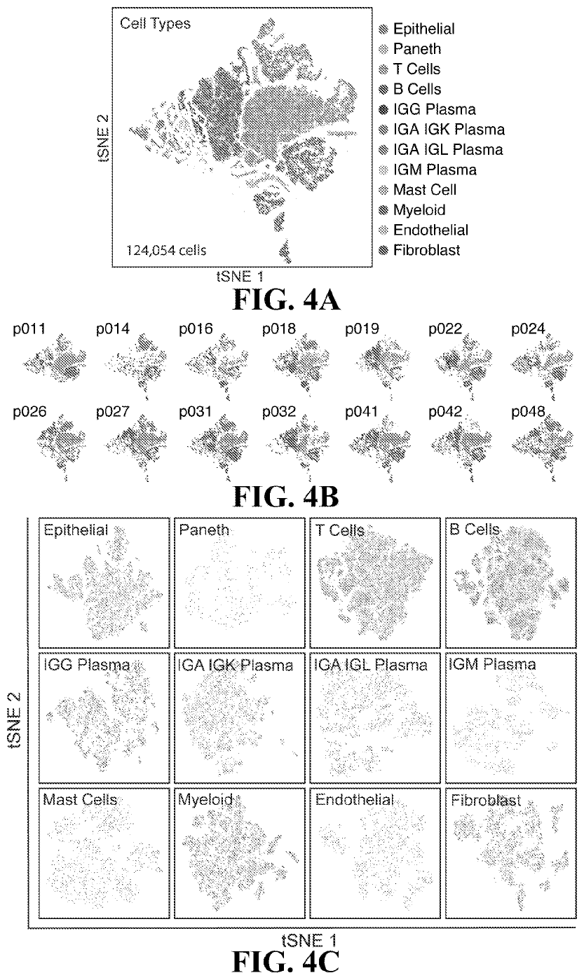

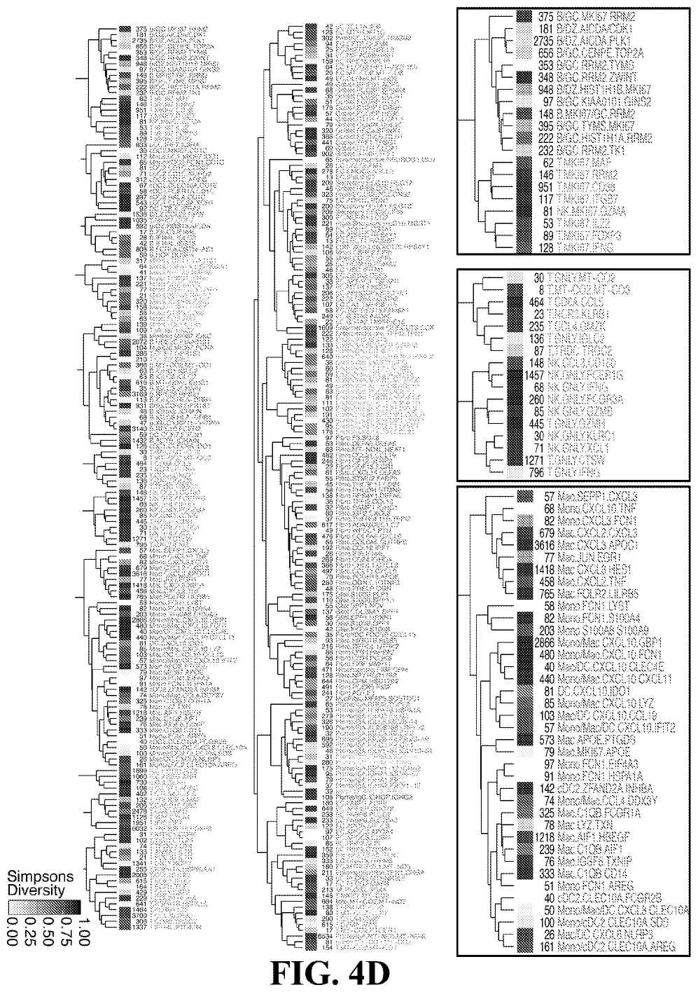

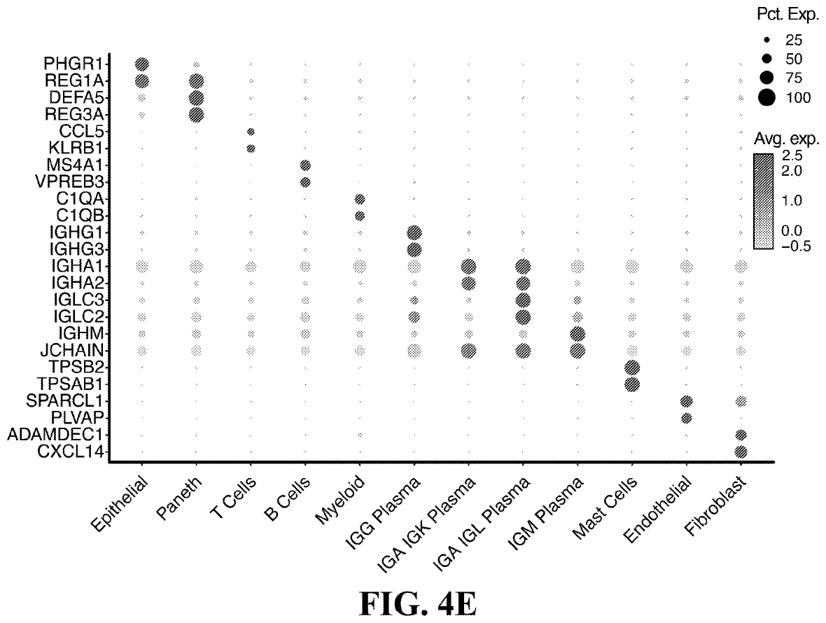

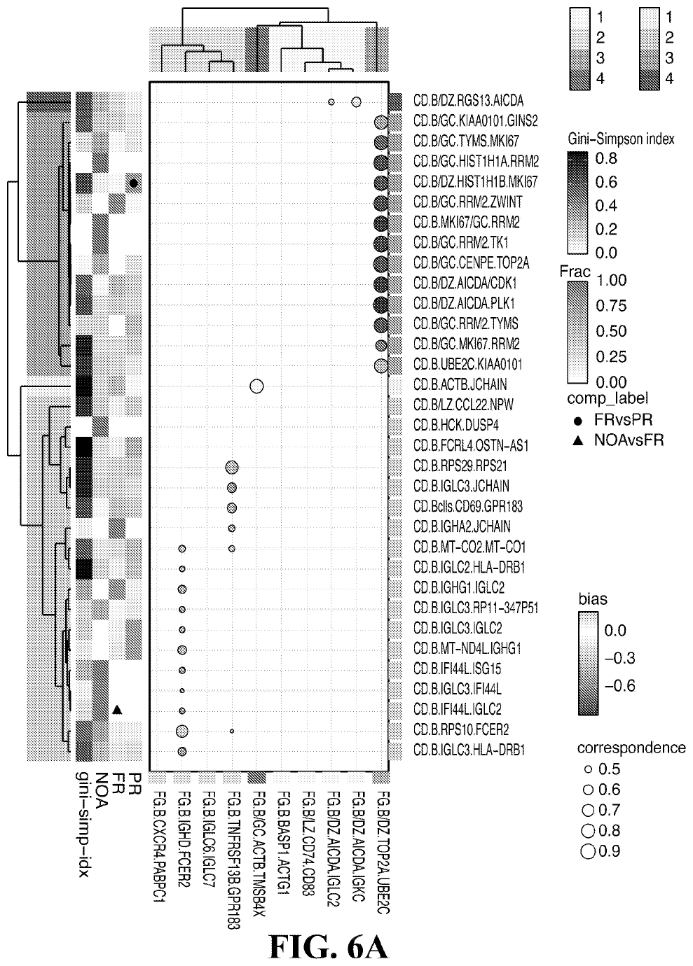

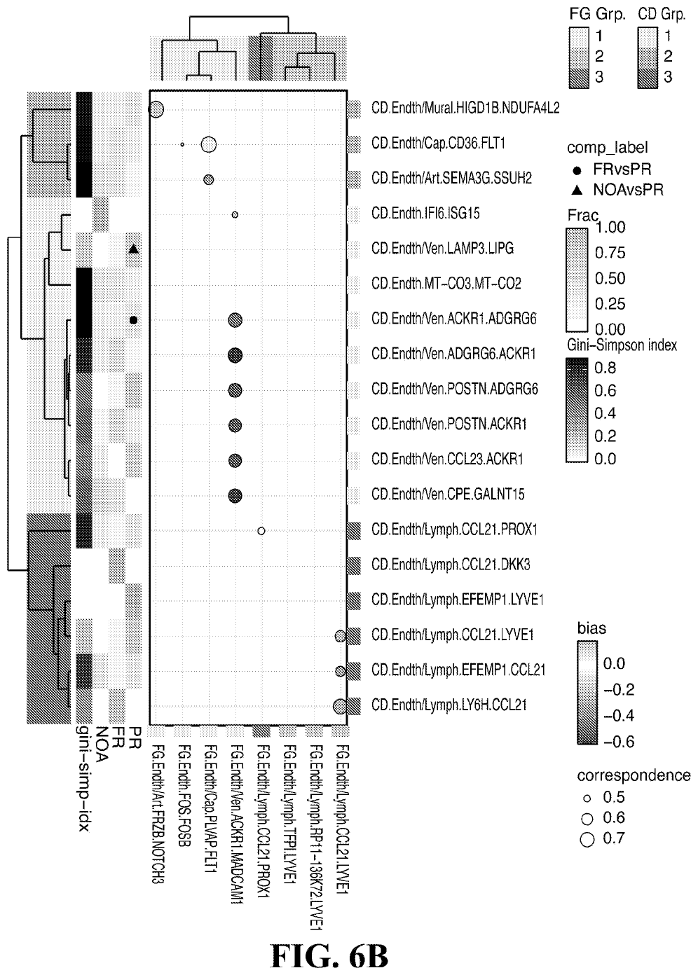

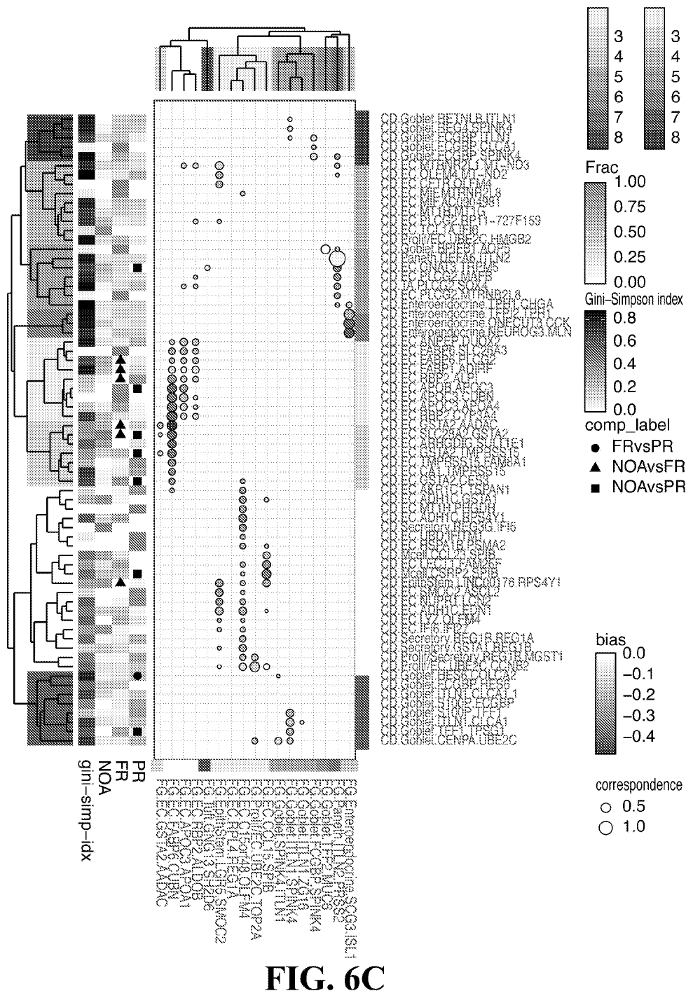

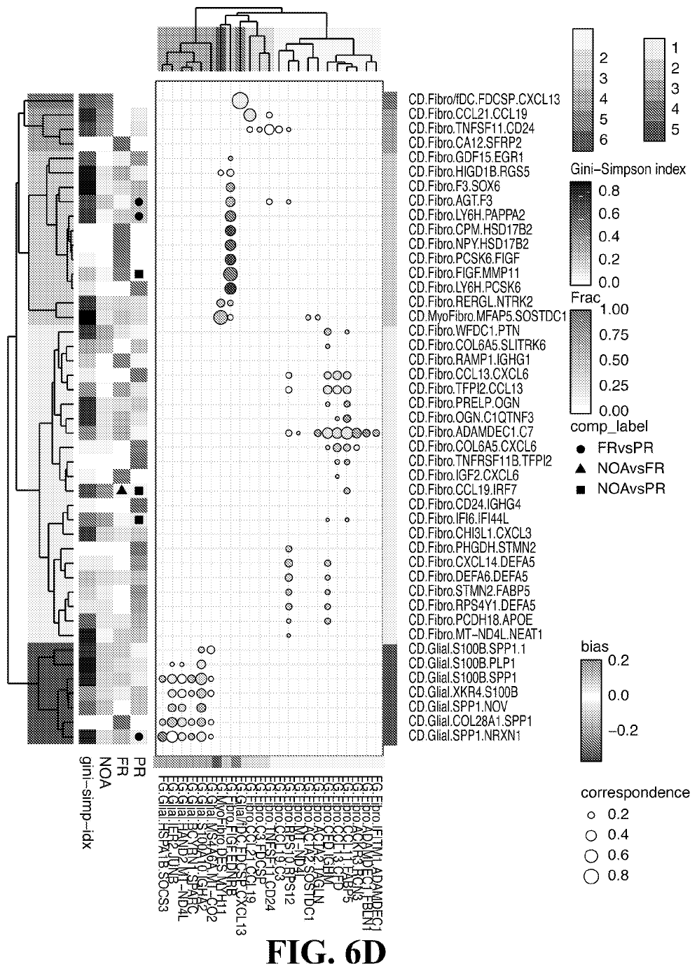

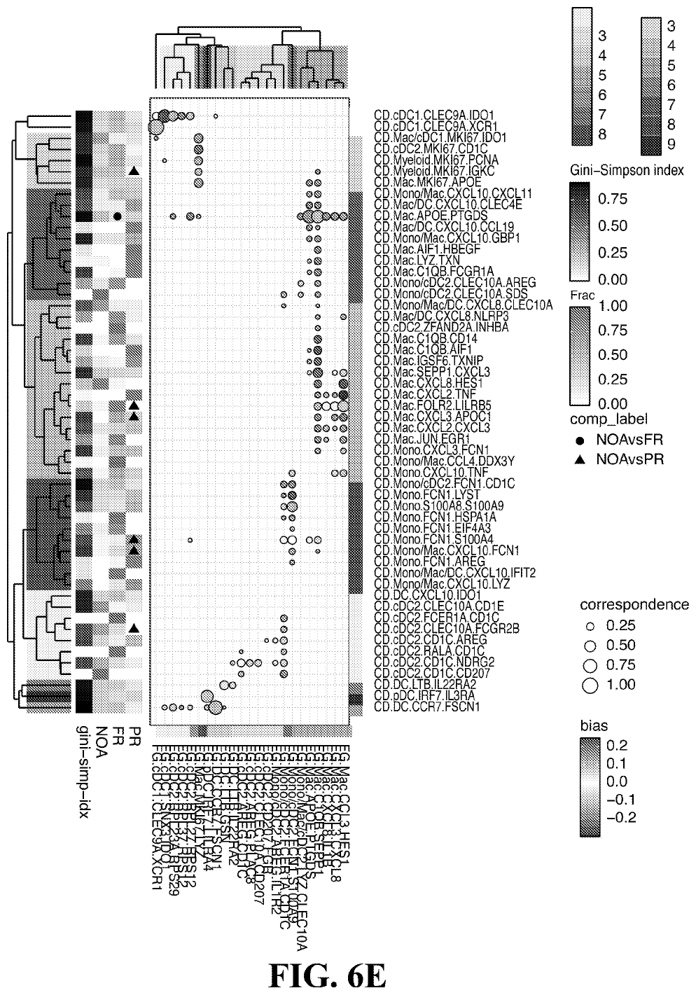

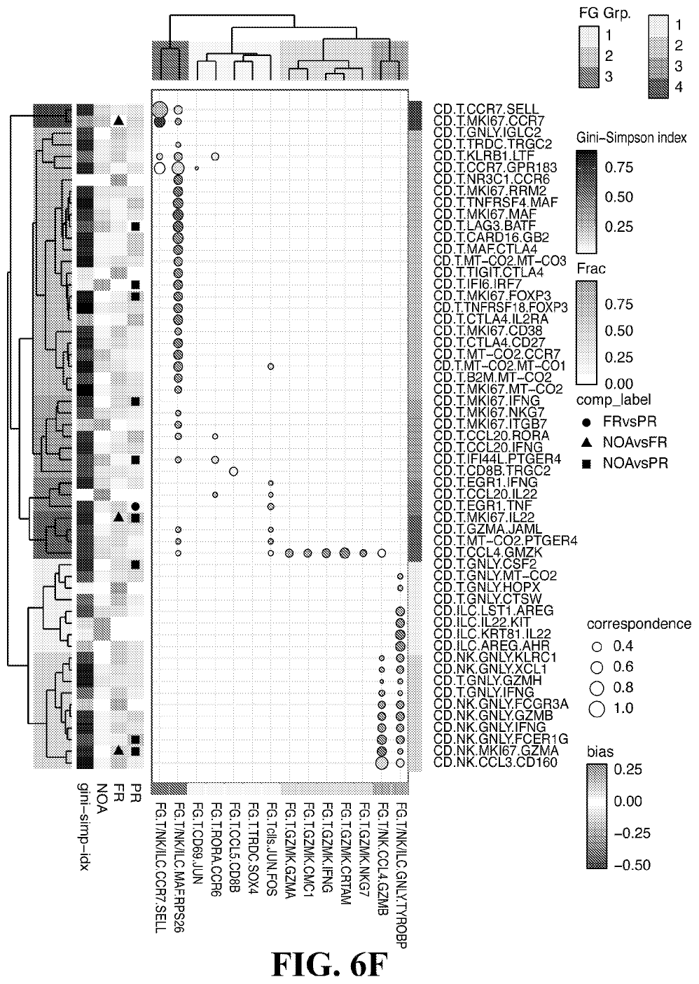

Using this analytical workflow, Applicants present two comprehensive cellular atlases of FGID (FIG. 3) and pediCD (FIG. 4), and then identify correspondence between the two (FIG. 6). Applicants provide gene lists for cell types (1 vs. rest across all cells), subsets (1 v. rest across all cells), and states (1 vs. rest within-cell-type) in Table 1 and Table 4. Applicants then focused on pediCD, and those cell states which distinguish between disease severity (NOA vs. PRs/FRs) and baseline gene expression differences in anti-TNF treatment response (FRs vs. PRs).

### Example 2—Comprehensive Atlas of Non-Inflammatory FGID

From the 99,488 cells profiled from 13 FGID patients, Applicants recovered 12 Tier 1 clusters which Applicants display on a t-stochastic neighbor embedding (t-SNE) plot colored by cluster identity (FIG. 3A). These Tier 1 clusters represent the main cell types found in the lamina propria and remnant epithelium of an ileal biopsy. Inspecting each individual patient's contribution to the t-SNE, Applicants noted that all patients contributed to all Tier 1 clusters, though note that p044 was overrepresented with more terminally differentiated epithelial cells, likely from incomplete EDTA separation, and thus omit the p044 unique cell clusters from further analyses of composition (FIG. 3B). Applicants then proceeded to generate preliminary descriptive names based on inspection of each cluster within each tier, calculated a hierarchically-clustered dendrogram, and then produced systematic names for each end cell state within each cell Tier1 cell type (FIGS. 3C, D; Methods). Applicants identified top marker genes for each main Tier 1 cluster/cell type, and note that Applicants also provide gene lists for Tier 1 clusters/cell types, subsets, and end cell states (FIG. 3E, Table 4). As patient identity did not factor into iterative tiered clustering stop conditions, Applicants then calculated Simpson's Index of Diversity to denote the patient diversity present within each end cluster, identifying that most clusters in FGID are conserved across multiple patients, and only a few clusters being recovered from an individual patient (FIG. 3D; Simpson's Index >0.25). These may still reflect important biology for the individual patient, but Applicants comment more extensively on clusters with high patient diversity.

Within B cells, Applicants identified a strong division between non-cycling and cycling B cells, with those found in the cycling compartment readily identifiable by germinal center markers and further dark zone (AICDA) and light zone (CD83) genes resulting in FG.B/DZ.AICDA.IGKC, and FG.B/LZ.CD74.CD83 clusters (FIG. 3D).

Within Myeloid cells, Applicants identified, and confirmed using extensive inspection of literature curated markers, cell subsets corresponding to monocytes (CD14, FCGR3A, FCN1, S100A8, S100A9, etc.), macrophages (CSF1R, MERTK, MAF, C1QA, etc.), cDC1 (CLEC9A, XCR1, BATF3), cDC2 (FCER1A, CLEC10A, CD1C, IRF4 etc.), and pDCs (IL3RA, LILRA4, IRF7) (FIG. 3D). Applicants highlight selected cell states including a migratory dendritic cell state (FG.DC.CCR7.FSCN1), extensive cDC2 heterogeneity relative to cDC1 heterogeneity, and a main distinction between macrophages expressing CIQ*, AMMP*, APOE, CD68 and PTGDS, (FG.Mac.C1Qb.SEPP1, FG.Mac.APOE.PTGDS) and a series of clusters expressing various chemokines including CCL3, CXCL3, CXCL8 (FG.Mac.CCL3.HES1, FG.Mac.CXCL3.CXCL8, FG.Mac.CXCL8.IL1B).

Within T cells, Applicants followed a similar approach as utilized for Myeloid cells and identified principal cell subsets of T cells (joint expression of CD247, CD3D, CD3E, CD3G with TRAC, TRBC1, TRBC2, or TRGC1, TRGC2 and TRDC), and a combined cluster of cytotoxic cells (FG.T/NK/ILC.GNLY.TYROBP) likely including T cells, NK cells (lower expression of TCR-complex genes with NCAM1, NCR1 and TYROBP), and some TLCs (KIT, NCR2, RORC and low expression of CD3-complex genes) (FIG. 3D). Applicants note that the numerical majority of CD4 T cells (FG.T/NK/ILC.MAF.RPS26) and CD8 T cells (FG.T/NK/ILC.CCR7.SELL) expressed SELL and CCR7 thus identifying them as naïve T cells. However, regardless of clusters expressing CD4 (FG.T.GZMK.GZMA) or CD8A/CD8B (FG.T.GZMK.IFNG, FG.T.GZMK.CRTAM, etc.), most activated T cells were characterized by expression of granzymes.

Within Epithelial cells, most cells expressed high levels of OLFM4, identifying them as crypt-localized cells. Applicants readily identified subsets of stem cells (LGR5), proliferating cells (TOP2A), goblet cells (SPINK4, ZG16, various MUCs), enteroendocrine cells (SCG3, ISL1), Paneth cells (ITLN2, PRSS2, LYZ), tuft cells (GNG13, SH2D6, TRPM5) and enterocytes (APOC3, APOA1, FABP6, etc.).

Within Endothelial cells, Applicants readily identified vascular and lymphatic endothelial cells (LYVE1, PROX1), with the vascular cells able to be further identified as capillaries (CA4) or venular endothelial cells (ACKR1, MADCAM1). Applicants also identified a subset of cells (FG.Endth/Peri.FRZB.NOTCH3) expressing high levels of FRZB and NOTCH3, which, rather than being arterioles, likely represent arteriole-associated pericytes or smooth muscle cells given the absence of EFNB2, SOX17, BMX, and HEY1, and the presence of ACTA2 and MYL9, as cluster-defining genes. Applicants highlight that the FG.Endth/Ven.ACKR1.MADCAM1 cluster is characterized by expression of markers for postcapillary venules specialized in leukocyte recruitment.

Within Fibroblasts, Applicants identified principal subsets characterized by their structural roles (COL3A1, ADAMDEC1, FBLN1, LUM, etc.), myofibroblasts (MYH11, ACTA2, ACTG2, etc.), and organization of lymphoid cells (CCL19, CCL21 etc.). Within the lymphoid-organizing fibroblasts, Applicants draw attention to the FG.Fibro.C3.FDCSP, FG.Fibro.CCL19.C3, and FG.Fibro,CCL21.CCL19 subsets, which appear to have some characteristics of follicular dendritic cells and variable expression of CCL19/CCL21 (T-cell or migratory dendritic cell chemoattractants) and CXCL13 (B-cell chemoattractant). Applicants also identified a separate Tier 1 cluster of Glial cells characterized by CRYAB and CLU. Intriguingly within the Glial cell Tier 1 cluster, Applicants then recovered a cell subset expressing FDCSP, CXCL13, and CR2, a key complement receptor which allows for complement-bound antigens to be recycled and presented by follicular dendritic cells. This highlights the power of iterative tiered clustering to recover discrete cell states that may, through the process of clustering not be fully resolved, and thus not identified and furthermore altering the gene signatures of their larger parent cell cluster. This FG.Glial/fDC.FDCSP.CXCL13 in the hierarchical cluster tree then assorts within the lymphoid-organizing stromal cells.

The Mast cells recovered did not further sub-cluster in an automated fashion, and were largely marked by TPSB2 and TPSAB1 (>97%), with minimal CMA1 (<20%) expressing cells, suggesting they are largely classical MC-T cells in FGID intestine.

Applicants identified four Tier 1 clusters for Plasma cells, which are characterized by their strong expression of IGH* immunoglobulin heavy-chain genes together with either a IGK* (kappa light chain) or IGL* (lambda light chain) genes. This resolved IgA IgK plasma cells, IgA IgL plasma cells, IgM plasma cells, and IgG plasma cells. Iterative tiered clustering identified further heterogeneity within all clusters of IgA and IgG plasma cells, though given the 3′-bias of this dataset, Applicants note that a principled investigation of these clusters would ideally use 5′ sequencing with targeted VDJ amplification.

Together, the treatment-naïve cell atlas from 13 FGID patients captures 118 cell clusters from a non-inflammatory state of pediatric ileum.

### Example 3—Comprehensive Atlas of CD

From the 124,054 cells profiled from 14 pediCD patients, Applicants recovered 12 Tier 1 clusters which here Applicants display on a t-stochastic neighbor embedding (t-SNE) plot colored by cluster identity, and represent the main cellular lineages found in the epithelium and lamina propria of an ileal biopsy (FIG. 4A). Distinct from FGID, Paneth cells clustered separately at Tier 1, while glial cells were now found within the Fibroblast Tier 1 cluster. Inspecting each individual patient's contribution to the t-SNE, Applicants noted that all patients contributed to all Tier 1 clusters (FIG. 4B). Applicants then proceeded to generate preliminary descriptive names, independently from the FGID atlas, based on inspection of each cluster within each tier, calculate a hierarchically-clustered dendrogram, and provide systematic names for each end cell state within each cell type and subset (FIGS. 4C, D; Methods). Applicants present top marker genes for each main Tier 1 cluster/cell type, and note the gene lists for Tier 1 clusters/cell types, subsets, and end cell states (Table 1). As patient identity did not factor into iterative tiered clustering stop conditions, Applicants then calculated Simpson's Index of Diversity to denote the patient diversity present within each end cluster, identifying that most clusters in pediCD are conserved across multiple patients, and only a few clusters being recovered from an individual patient (FIG. 4D; Simpson's Index>0.25). Applicants note that in pediCD, relative to FGID, a higher fraction of cell clusters exhibited lower patient diversity. These may still reflect important biology for the individual patient, but Applicants comment more extensively on clusters composed of high patient diversity.

Within B cells, Applicants also identified a strong division between non-cycling and cycling B cells, with those found in the cycling compartment readily identifiable by germinal center markers and further dark zone (AICDA) and light zone (CD83) genes, as in FGID. Within cells expressing germinal centers markers, a highly-proliferative branch including clusters such as CD.B/LZ.CCL22.NPW, CD.B/GC.MKI67.RRM2, and CD.B/DZ.HIST1H1B.MKI67 emerged (FIG. 4D). The CD.B/LZ.CCL22.NPW was characterized by high levels of MYC, which has recently been shown to provide an inertial cue to allow for further rounds of germinal center affinity maturation. More numerous B cell clusters included ones characterized by expression of GPR183, such as CD.B.CD69.GPR183 (also expressing IGHG1) and CD.B.RPS29.RPS21. GPR183 has been shown to regulate the positioning of B cells in lymphoid tissues.

Within Myeloid cells, Applicants identified, and confirmed using the same extensive inspection of literature curated markers as in FGID, cell subsets corresponding to monocytes (CD14, FCGR3A, FCN1, S100A8, S100A9, etc.), macrophages (CSF1R, MERTK, MAF, C1QA, etc.), cDC1 (CLEC9A, XCR1, BATF3), cDC2 (FCER1A, CLEC10A, CD1C, IRF4 etc.), and pDCs (IL3RA, LILRA4, IRF7) (FIG. 4D). Applicants highlight selected cell states including a migratory dendritic cell state (CD.DC.CCR7.FSCN1), extensive cDC2 heterogeneity relative to cDC1 heterogeneity, and a main distinction between macrophages expressing CIQ*, AI*IP, APOE, CD68 and PTGDS, (CD.Mac.APOE.PTGDS) and a series of clusters expressing various chemokines including CXCL2, CXCL3, and CXCL8 (CD.Mac.SEPP1.CXCL3, CD.Mono.CXCL3.FCN1, CD.Mono.CXCL10.TNF). Several of the end cell clusters initially clustering with macrophages also expressed monocyte markers (S100A8, S100A9), and expressed detectable, but lower levels of MERTK or AXL relative to bona fide macrophages, potentially indicative of the early stages of monocyte-to-macrophage differentiation. Applicants also noted a substantial expansion of clusters characterized by expression of CXCL9, CXCL10, and STAT1, canonical interferon-stimulated genes, seen in clusters such as CD.Mono/Mac.CXCL10.FCN1. Applicants identified a cluster of inflammatory monocytes, CD.Mono.S100A8.S100A9, characterized by both CD14 and FCGR3A expression.

Within T cells, Applicants followed a similar approach as utilized for FGID T cells and identified cell subsets of T cells (joint expression of CD247, CD3D, CD3E, CD3G with TRAC, TRBC1, TRBC2, or TRGC1, TRGC2 and TRDC), but in pediCD also identified several discrete clusters of NK cells (lower expression of TCR-complex genes with FCGR3A or NCAM1, NCR1 and TYROBP), and ILCs (KIT, NCR2, RORC and low expression of CD3-complex genes) (FIG. 4D). Applicants note that T cells and NK cells with a shared expression of GNLY, GZMB and other cytotoxic effector genes cluster almost indistinguishably from each other through iterative tiered clustering and visualization of the hierarchical tree, but that careful inspection of literature-curated markers helped resolve NK cells (CD.NK.CCL3.CD160; CD.NK.GNLY.GZMB) from CD8A/CD8B T cells (CD.T.GNLY.GZMH; CD.T.GNLY.CTSW). One of the specific challenges in distinguishing between T cells and NK cells in scRNA-seq data is that NK cells can express several CD3-complex genes, particularly CD247, as well as detectable aligned reads for TRDC or TRBC1 and TRBC2, and thus lower-resolution clustering approaches or datasets with lower cell numbers may miss these important distinctions. NK cell clusters also expressed the highest levels of TYROBP, which encodes DAP12 and mediates signaling downstream from many NK receptors. ILC clusters such as CD.ILC.LST1.AREG or CD.ILC.IL22.KIT were characterized by an apparent ILC3 phenotype, with expression of KIT, RORC and IL22, though they also expressed detectable transcripts of GATA3 in the same clusters. Applicants detected several clusters expressing CD4 and lacking CD8A CD8B, including regulatory T cells (CD.T.TNFRSF18.FOXP3), and MAF- and CCR6-expressing helper T cells (CD.T.MAF.CTLA4). Perhaps most strikingly, Applicants resolved multiple subsets of proliferating lymphocytes, including regulatory T cells (CD.T.MKI67.FOXP3), IFNG-expressing T cells (CD.T.MKI67.IFNG), and NK cells (CD.NK.MKI67.GZMA).

Within Epithelial cells, most cells expressed high levels of OLFM4 as well, identifying them as crypt-localized cells. Applicants readily identified subsets of stem cells (LGR5), proliferating cells (TOP2A), goblet cells (SPINK4, ZG16, various MUCs), enteroendocrine cells (SCG3, ISL1), Paneth cells (ITLN2, PRSS2, LYZ), tuft cells (GNG13, SH2D6, TRPM5) and enterocytes (APOC3, APOA1, FABP6, etc.). Amongst several clusters characterized by CCL25 and OLFM4 expression, Applicants identified a subset marked by LGR5 expression, characteristic of intestinal stem cells (CD.EpithStem.LINC00176.RPS4YA1). Applicants identified several subsets expressing CD24, indicative of crypt localization, with expression of REG1B (CD.Secretory.GSTA1.REG1B; CD.Secretory.REG1B.REG1A). Applicants also identified early enterocyte cluster CD.EC.ANPEP.DUOX2, characterized by FABP4 and ALDOB and expressing DUOX2 and MUC1. Applicants resolved several clusters of enteroendocrine cells, including CD.Enteroendocrine.TFPI2.TPH1 and CD.Enteroendocrine.NEUROG3.MLN. Applicants also found two clusters Applicants labeled as M cells based on expression of SPIB (CD.Mcell.CCL23.SPIB; CD.MCell.CSRP2.SPIB). Paneth cells did not further sub-cluster despite forming an independent Tier 1 cluster (CD.Epith.Paneth). Most strikingly, Applicants identified a diversity of goblet cells recovered across multiple patients including CD.Goblet.HES6.COLCA2 expressing REG4 and LGALS9, and CD.Goblet.TFF1.TPSG1 expressing TFF1 and ITLN1, amongst others. Applicants also identified a cluster of Tufts cells: CD.EC.GNAT3.TRPM5.

Within Endothelial cells, Applicants readily identified vascular and lymphatic endothelial cells (LYVE1, PROX1), with the vascular cells able to be further identified as capillaries (CA4) or venular endothelial cells (ACKR1, MADCAM1). Applicants also identified a subset of cells (FG.Endth/Peri.FRZB.NOTCH3) expressing high levels of FRZB and NOTCH3, which, rather than being arterioles, likely represent arteriole-associated pericytes or smooth muscle cells given the absence of EFNB2, SOX17, BMX, and HEY1, and the presence of ACTA2 and MYL9, as cluster-defining genes. In pediCD, Applicants also identified a cluster of arteriole endothelial cells, CD.Endth/Art.SEMA3G.SSUH2, identified by expression of HEY1, EFNB2, and SOX17. Applicants also highlight that the endothelial venules characterized by expression of markers for postcapillary venules specialized in leukocyte recruitment, such as CD.Endth/Ven.ADGRG6.ACKR1 and CD.Endth/Ven.POSTN.ACKR1, exhibited greater diversity than in FGID with multiple end cell clusters identified.

Within Fibroblasts, Applicants identified principal subsets characterized by their structural roles (COL3A1, ADAMDEC1, FBLN1, LUM, etc.), myofibroblasts (MYH11, ACTA2, ACTG2, etc.), and organization of lymphoid cells (CCL19, CCL21 etc.). The principal hierarchy in fibroblasts in pediCD was between FRZB-, EDRNB- and F3-expressing subsets such as CD.Fibro.LY6H.PAPPA2 and CD.Fibro.AGT.F3, which were also enriched for CTGF and MMP1 expression, and ADAMDEC1-expressing fibroblasts, which were enriched for several chemokines such as CXCL12, and in some specific clusters CXCL6, CXCL1, CCL11, and other chemokines. Amongst three fibroblast subsets marked by C3 expression, Applicants identified follicular dendritic cells (CD.Fibro/fDC.FCSP.CXCL13), along with fibroblasts expressing CCL21, CCL19, and the interferon-stimulated chemokines CXCL9 and CXCL10 (CD.Fibro.CCL21.CCL19; CD.Fibro.TNFSF11.CD24). Distinct from the FGID atlas, within the pediCD atlas, glial cells clustered within fibroblasts, but were also marked by S100B, PLP1 and SPP1 expression. Applicants note that many fibroblasts were found with T cells, generating extensive doublet clusters.

The Mast cells recovered in pediCD did further sub-cluster in an automated fashion, were largely marked by TPSB2 (>90%), with minimal CMA1 (<16%) expressing cells, suggesting they are largely classical MC-T cells in pediCD intestine. Intriguingly, some subsets (CD.Mstcl.AREG.ADCYAP1) were enriched for IL13-expression. Applicants also detected a small cluster of proliferating mast cells from several patients (CD.Mstcl.CDK1.KIAA0101).

Applicants also identified four Tier 1 clusters for Plasma cells, which are characterized by their strong expression of IGH* immunoglobulin heavy-chain genes together with either a IGK* (kappa light chain) or IGL* (lambda light chain) genes. This resolved IgA IgK plasma cells, IgA IgL plasma cells, IgM plasma cells, and IgG plasma cells. Iterative tiered clustering identified further heterogeneity within all clusters of IgA plasma cells, though given the 3′-bias of this dataset, Applicants note that a principled investigation of these clusters would ideally use 5′ sequencing with targeted VDJ amplification.

Together, the treatment-naïve cell atlas from 14 pediCD patients captures 305 cell clusters from an inflammatory state of pediatric ileum.

### Example 4—Clinical Variables and Cellular Variance that Associates with pediCD Severity

Because this pediCD atlas was curated from treatment-naïve diagnostic samples, Applicants were able to interrogate the data to determine to test if overall shifts in cellular composition, specific cell states, and/or gene expression signatures underlie clinically-appreciated disease severity and treatment decisions (NOA vs. FR/PR), and those that are associated with either FRs or PRs to anti-TNF blockade. Here, Applicants leveraged the detailed clinical trajectories collected from all patients in order to resolve distinctions between cellular composition and cell states with disease and treatment outcomes.

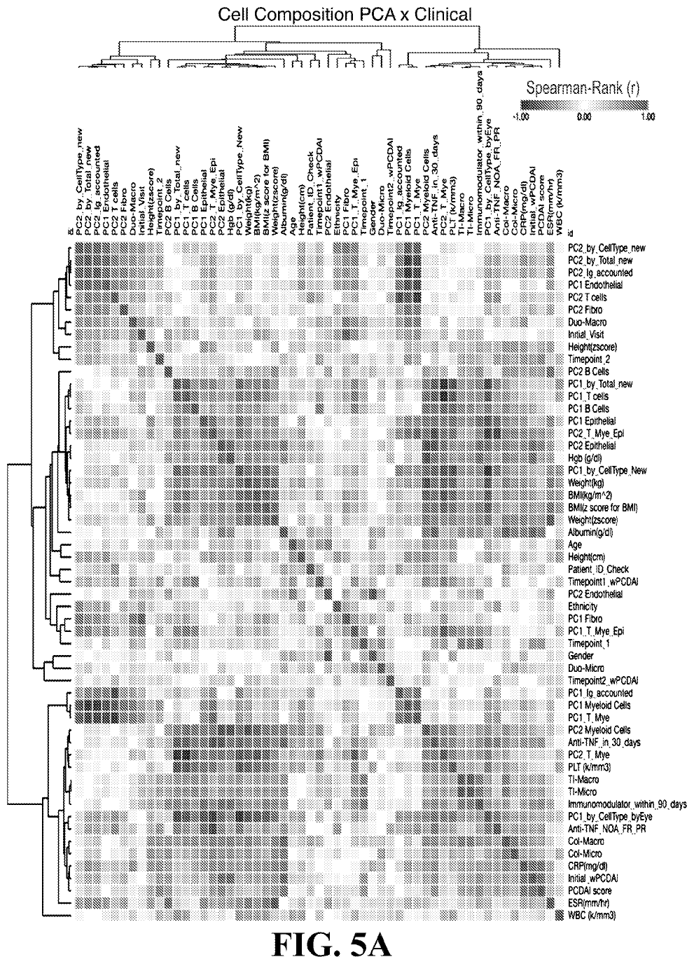

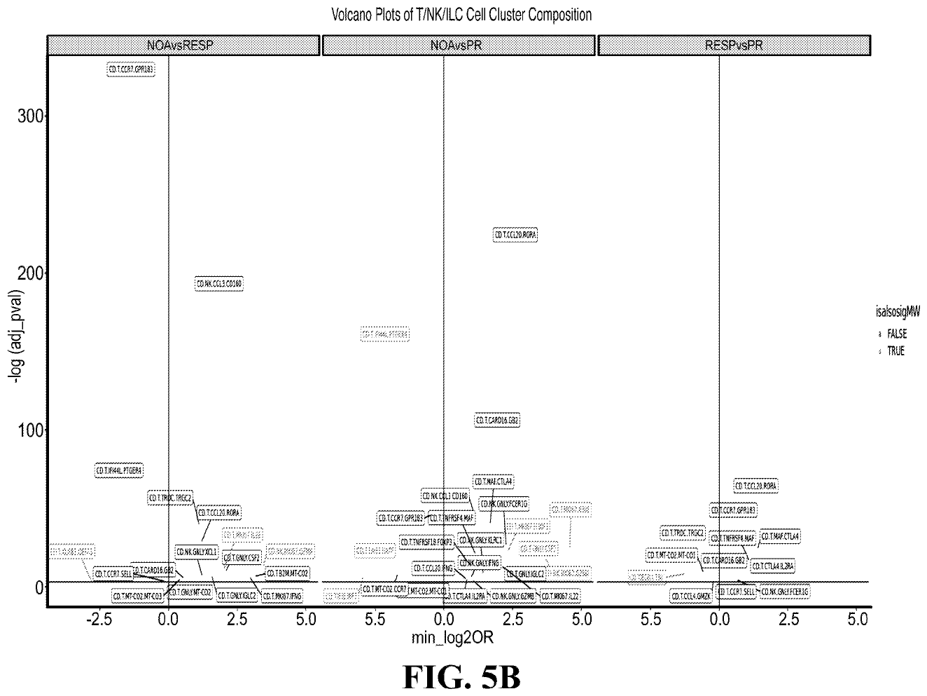

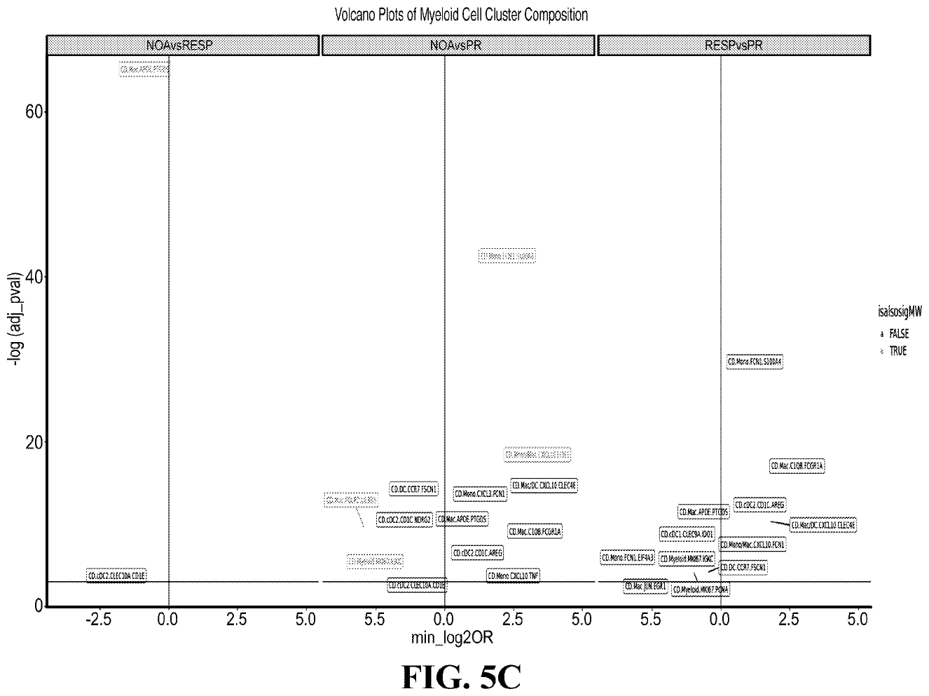

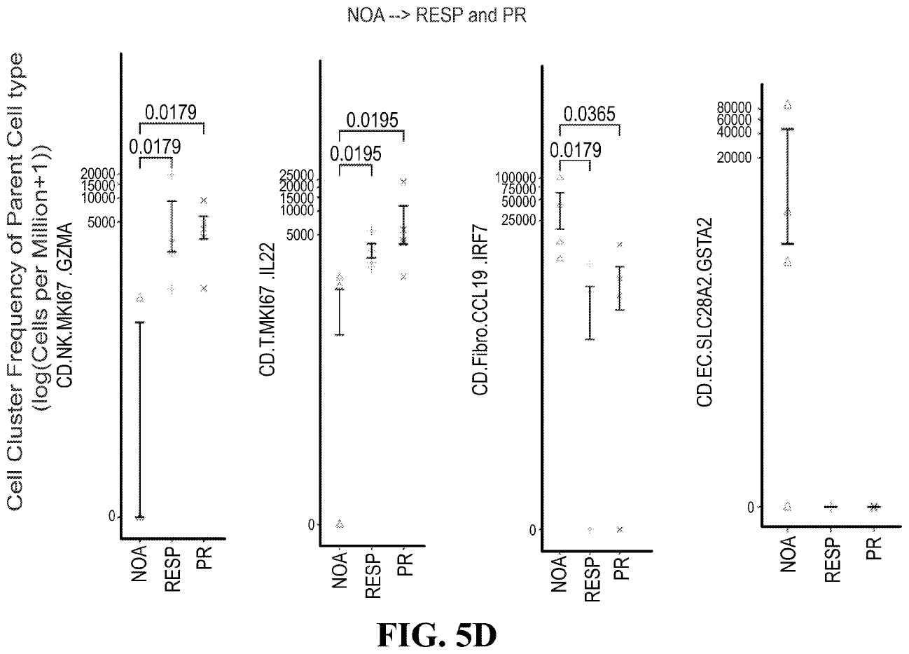

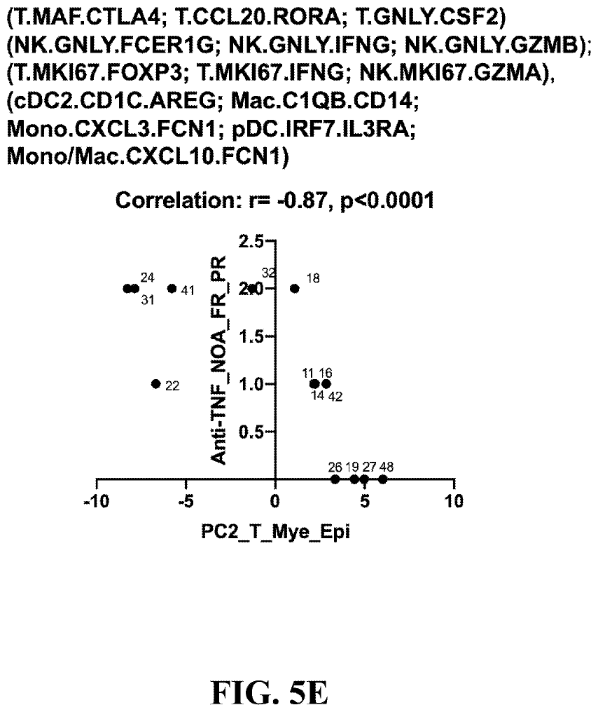

In order to capture the overall principal axes of variation explaining changes in cellular composition, Applicants calculated the fractional composition of all 305 end cell clusters in pediCD within its parent cell type (“per cell type”), or within all cells (“per total cells”), and performed a principal component analysis (PCA) over both of these sample x cell cluster frequency tables. Applicants then used the PC1 (13.4% variation “per cell type” and 13.5% variation “per total cells”) and PC2 (12.7% variation “per cell type” and 11.8% variation “per total cells”) as numerical variables which Applicants correlated with clinical metadata including categorical variables (patient ID, ethnicity, gender, etc.), ordinal variables (TI-macroscopic, TI-microscopic, Anti-TNF in 30 days, anti-TNF_NOA_FR_PR, etc.) and numerical variables (Height, BMI, CRP, ESR, PLT, PCDAI (Pediatric Crohn's Disease Activity Index), wPCDAI, etc.) (FIG. 5, r by Spearman-rank). Amongst the clinical variables, Applicants noted strong correlation with Initial wPCDAI and CRP (r>0.83), and moderate correlation with Initial wPCDAI and anti-TNF in 30 days (r=0.65) and anti-TNF_NOA_FR_PR (r=0.49). For PC1-“per total cells”, Applicants identified strong correlations with anti-TNF treatment within 30 days of diagnosis (r=−0.76), and moderate correlation with treatment decision/response defined as NOA, FR or PR and coded as anti-TNF_NOA_FR_PR (r=−0.58; Methods). For PC1-“per cell type”, Applicants identified strong correlation with the decision to place patients on anti-TNF within 30 days of diagnosis (r=−0.72), and moderate with anti-TNF_NOA_FR_PR status (r=−0.63). PC1-“per cell type” was also strongly correlated with BMI and PC1-“per total cells” (r>−0.7). PC1-“per cell type” was weakly correlated with patient ID and gender.

In order to understand if any cell types were predominantly driving associations with disease severity, Applicants then further decomposed the overall PCA on 305 end clusters and performed PCA over each cell type's fractional composition of end clusters individually (B cells: 33 clusters, Endothelial: 18 clusters, Epithelial: 68 clusters, Fibroblast 45 clusters, Myeloid: 54 clusters, T cells: 57 clusters), and correlated the first two PC's (all PC1's and PC2's accounted for >13% variance each) with all of the clinical variables. The PCs derived from T/NK/ILC cells, Myeloid cells, and Epithelial cells were all moderately correlated with anti-TNF_NOA_FR_PR status (>0.49) and had higher values than the other cell types, so Applicants asked if a PCA-based metric considering all three cell types would capture disease severity and treatment response. When Applicants calculated the PCA accounting for frequencies within each cell type of T/NK/ILC cells, Myeloid cells, and Epithelial cells, Applicants found strong correlation for PC2 with both anti-TNF within 30 days (r=−0.83) and anti-TNF-NOA_FR_PR status (r=−0.87) (FIG. 5E). This represented the two strongest correlations of any variable Applicants tested with anti-TNF prescription and response status, outperforming wPCDAI, and again were weakly correlated with patient ID, ethnicity, and gender. Some of the top negative loadings for PC2 included both helper and cytotoxic T cell clusters (T.MAF.CTLA4; T.CCL20.RORA; T.GNLY.CSF2), NK cell clusters (NK.GNLY.FCER1G; NK.GNLY.IFNG; NK.GNLY.GZMB); proliferating T cells and NK cells (T.MKI67.FOXP3; T.MKI67.IFNG; T.MKI67.IL22; NK.MKI67.GZMA), and monocytes, macrophages, DCs and pDCs (cDC2.CD1C.AREG; Mac.C1QB.CD14; Mono.CXCL3.FCN1; pDC.IRF7.IL3RA; Mono/Mac.CXCL10.FCN1) (Table 3). This suggests that multiple collective changes in the composition and/or state of T/NK/ILC cells, Myeloid cells, and Epithelial cells at diagnosis may help stratify pediCD patients not only by clinically appreciated disease severity but also influence anti-TNF responsiveness.

### Example 5—Changes in Cell State Composition Across Disease Severity Spectrum

Applicants next focused on further deconstructing this severity vector: identifying which cell clusters accounted for the most significant changes in abundance based on the relative frequency of an end cell cluster within its parent cell type. Applicants focus on this form of analysis, as may typically be reported for flow cytometry, and further discuss approaches to enumerate total cell numbers which would be critical to identify changes in overall cellularity in the different pediCD treatment and response categories (Discussion). Applicants first performed a Fisher's exact test between NOA vs. FR, NOA vs. PR or FR vs. PR, and then performed a Mann-Whitney U test to highlight specific clusters and discuss results from clusters with high Simpson's index of diversity (i.e. recovered from multiple patients) as shown for T/NK/ILCs and Myeloid Cell Types (FIG. 5B,C).

Cell Subsets that are NOA→RESP and PR

Between NOAs and both FRs and PRs, two subsets with significantly increased frequency amongst T cells, NK cells, and ILCs were identified. These were CD.NK.MKI67.GZMA and CD.T.MKI67.IL22 (FIG. 5A, D). Beyond the strong proliferation signature, CD.NK.MKI67.GZMA were enriched for genes such as GNLY, CCL3, KLRD1, IL2RB and EOMES, and CD.T.MKI67.IL22 were enriched for IFNG, CCL20, IL22, IL26, CD40LG and ITGAE. This indicates that with increasing pediCD severity, there is increasing local proliferation of cytotoxic NK cells, and tissue-resident T cells with the capacity to express anti-microbial and tissue-reparative cytokines, and molecules to interface with antigen-presenting cells and B cells. Alongside this increase, there was a significant decrease amongst fibroblasts of CD.Fibro.CCL19.IRF7, and amongst epithelial cells of CD.EC.SLC28A2.GSTA2 clusters (FIG. 5A, D). The CD.Fibro.CCL9.IRF7 were enriched for CCL19, CCL11, CXCL1, CCL2, and very specifically for OAS1 and IRF7. The CD.EC.SLC28A2.GSTA2 cluster was characterized by its two namesake markers, involved in purine transport and glutathione metabolism.

Cell subsets that are NOA→PR

Applicants next focused on those cell subsets that were significantly changed only between NOAs and PRs. Here Applicants note several more distinct clusters within the lymphocyte cell type, including increases of CD.T.MKI67.IFNG, CD.T.MKI67.FOXP3, CD.T.GNLY.CSF2, and CD.NK.GNLY.FCER1G in the PR patients compared to NOA patients. The two MKI67 clusters again highlighted an increase in proliferative cells, specifically cells enriched for IFNG, GNLY, HOPX, ITGAE and IL26 (CD.T.MKI67.IFNG), and IL2RA, BATF, CTLA4, TNFRSF1B, CXCR3, and FOXP3 (CD.T.MKI67.FOXP3), the latter of which may be indicative of proliferating regulatory T cells. The two GNLY clusters emphasized cytotoxicity, specifically cell clusters were both enriched for GNLY, GZMB, GZMA, PRF1 and more specifically for IFNG, CXCR6, and CSF2 (CD.T.GNLY.CSF2), or AREG, TYROBP, and KLRF1 (CD.NK.GNLY.FCER1G). Amongst myeloid cells, there was an increase in CD.Mac.CXCL3.APOC1, CD.Mono/Mac.CXCL10.FCN1, and CD.Mono.FCN1.S100A4 in PR versus NOA. The CD.Mac.CXCL3.APOC1 cluster was enriched for a variety of chemokines including CCL3, CCL4, CXCL3, CXCL2, CXCL1, CCL20, and CCL8. It was also enriched for TNF and IL1B. The CD.Mono/Mac.CXCL10.FCN1 cluster was enriched for CXCL9, CXCL10, CXCL11, GBP1, GBP2, GBP4, GBP5, suggestive of activation by IFN, and more specifically Type II IFN-gamma, based on the GBP gene cluster. CD.Mono.FCN1.S100A4 was characterized by S100A4, S100A6, and FCN1 expression. These two hematopoietic clusters were paralleled by increases in certain clusters within endothelial cells (CD.Endth/Ven.LAMP3.LIPG) and epithelial cells (CD.Goblet.TFF1.TPSG1).

Several clusters of cells were decreased in PR versus NOA, including CD.T.LAG3.BATF, CD.T.IFI44L.PTGER4, and CD.T.IFI6.IRF7 amongst lymphocytes. Amongst myeloid cells, CD.cDC2.CLEC10A.FCGR2B were decreased, and amongst fibroblasts CD.Fibro.IFI6.IFI44L were decreased. In epithelial cells, CD.Tuft.GNAT3.TRPM5 cells were decreased. Alongside the decrease in Tuft cells amongst epithelial cells, two more clusters closely related to the aforementioned CD.EC.GSTA2.SLC28A3 cluster, also marked by GSTA2 expression, were significantly decreased (CD.EC.GSTA2.CES3, and CD.EC.GSTA2.TMPRSS15).

Cell Subsets that are NOA→RESP

Applicants also detected significant decreases in FRs relative to NOAs in certain cell types, particularly within Epithelial cells including CD.EpithStem.LINC00176.RPS4Y1, CD.MCell.CSRP2.SPIB, CD.EC.FABP6.PLCG2, and CD.EC.FABP1.ADIRF. Applicants note that the relative decrease in M cells is in stark contrast to the “ectopic” M-like cells that were detected in adult ulcerative colitis.

Cell Subsets that are FR→PR

Lastly, Applicants assessed the compositional differences between FRs and PRs and only identified one cell cluster which was significantly increased in PRs: CD.B/DZ.HIST1H1B.MKI67, which are proliferating dark zone B cells. Together, these data suggest that at the earlier stages of pediCD, there are a series of gradual changes in the multiple cell types that encapsulate the progression from NOA to FR to PR patients. These changes were particularly notable within proliferating T cells, cytotoxic NK cells, and monocytes/macrophages that together provide a numerical variable in PC2-“T/NK/ILC/Myeloid/Epithelial” which correlates strongly with both anti-TNF use within 30 days and anti-TNF_NOA_FR_PR status.

### Example 6—Random Forest Classifier Applied to Cellular Taxonomies Allows for Identification of Correspondence Between FGID and pediCD

As Applicants had generated independent cellular atlases for FGID and pediCD, Applicants next sought to identify correspondence between jointly detected cell subsets. As the study progressed, several analytical methods to integrate scRNA-seq emerged which utilize distinct principles to either predict cell type names given reference gene lists or directly integrate two datasets that were collected from distinct perturbations, tissue, or even species. However, many of these methods are benchmarked on broad cell type or subset integration, and thus their applicability for fine cell states, as in the end clusters Applicants identify here, remains unknown. Thus, Applicants employed a random forest classifier based approach, which has recently also been applied successfully in work to identify correspondence in fine sub-clusters in the mammalian retina. Specifically, Applicants employed cross validation within FGID or pediCD cell types before running between FGID and pediCD in both directions (Methods). Applicants applied this to all cell types, and here focus the discussion on Myeloid cells and T/NK/ILC cells (FIG. 6). As newer methods are developed, more refined integration is likely to be possible.

Comparing across Myeloid cells between pediCD and FGID, Applicants could identify strong correspondence of specific cell subsets such as cDC1's and pDCs (FIG. 6). Applicants also identified strong correspondence between several cDC2 clusters. Applicants identified a gradient of monocyte and macrophage correspondence to two FGID clusters by 31 clusters in pediCD, likely reflective of inflammatory monocyte to macrophage differentiation. Some clusters characterized by STAT1 activation did not demonstrate significant correspondence to any FGID cluster. Applicants also generally noted substantially increased cluster diversity in pediCD end clusters relative to their correspondence in FGID. This emerged from more patient-specific clusters found in pediCD, and an overall decrease in Simpson's index of diversity considering the patient composition of each end clusters.

For T/NK/ILLC cells, Applicants identified more discrete patterns relative to Myeloid cells based on comparison of the Random Forest result. Within the two FGID cytotoxic T cell clusters, Applicants identified correspondence by 18 pediCD clusters, representing Type 17 ILCs, and cytotoxic NK cells and T cells (FIG. 6). The cluster of naïve T cells in FGID had correspondence with the majority of pediCD non-cytotoxic T cell clusters, illustrating a substantial activation and specialization to several discrete T cell states.

Importantly, when Applicants jointly clustered macrophages from FGID and pediCD together, Applicants identified that several of the original end clusters identified to be through iterative tiered clustering in pediCD were divided across the UMAP, ending split up in distinct clusters of cells. This highlights the challenge of multiple cell type, subset, and state vectors which are simultaneously accounted for by clustering over a set of highly variable genes jointly derived from multiple cells and disease conditions, and that highly homogenous cell clusters may be dispersed across a space based on the other cells that they are being compared against and the parameters used for clustering.

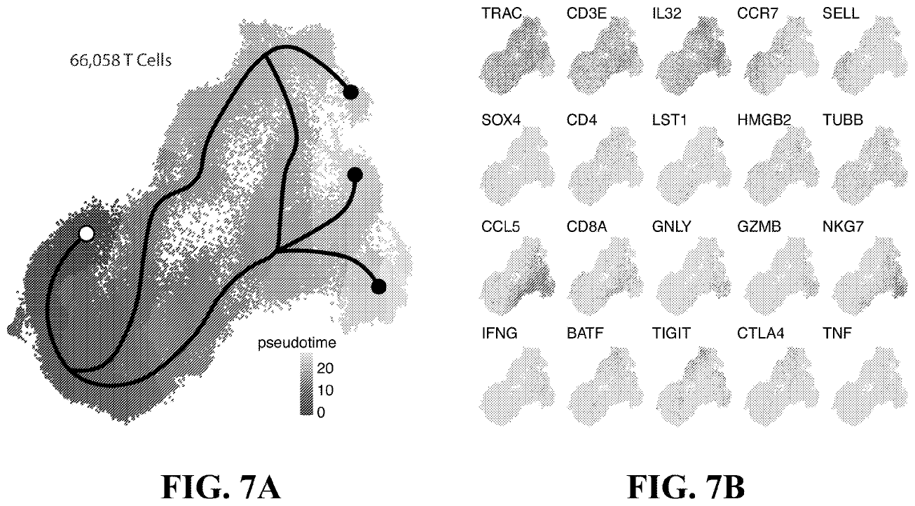

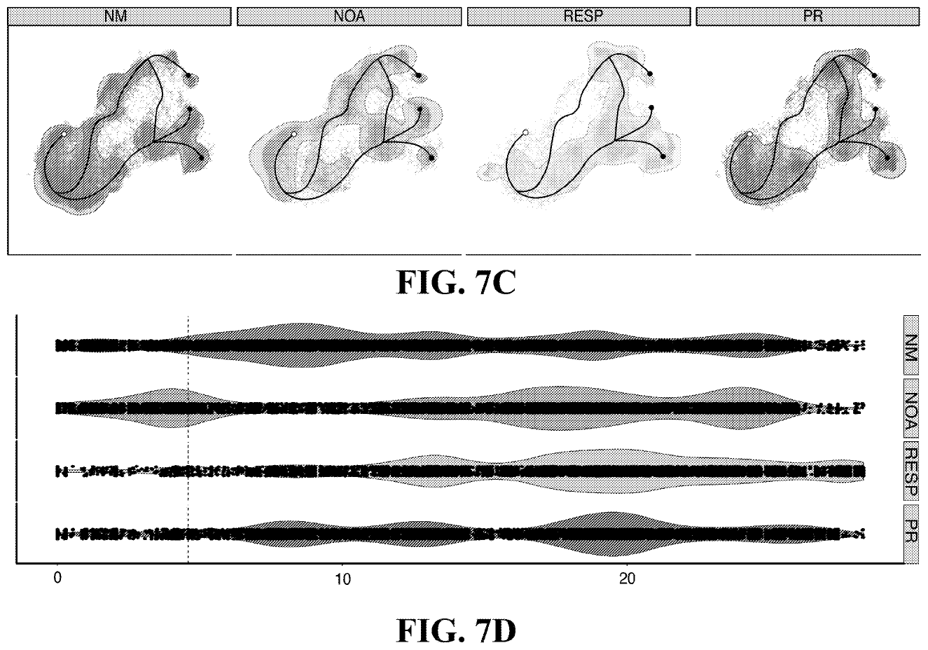

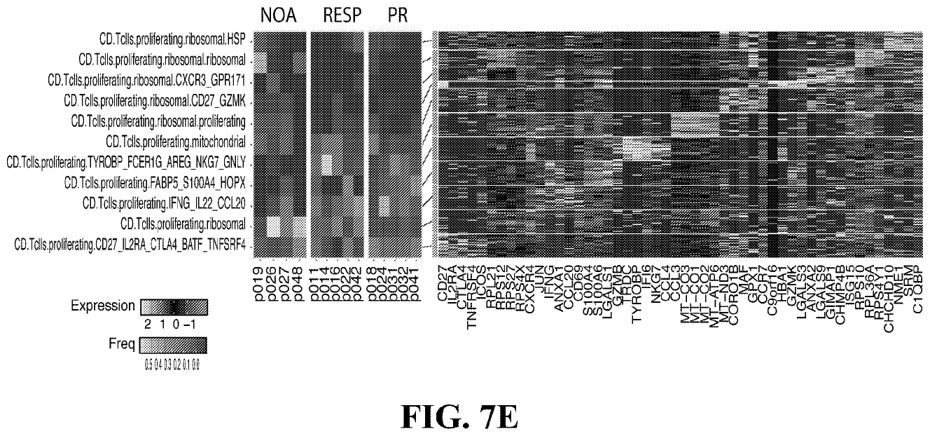

Based on their over-representation within clusters showing more significant differences within pediCD, Applicants then focused on performing pseudotime over a shared gene expression space of the T/NK/ILCs and monocytes/macrophages. Applicants utilized a list of genes that were cell-type defining genes in either FGID or pediCD (Table 1 and Table 4), but removed genes that were differentially-expressed between FGID and pediCD (Table 2), to allow for cell type/subset to drive placement on the pseudotime axis (Methods). This allowed Applicants to place the fine-grained clusters within a joint gene-expression space to relate FGID to pediCD. In the T/NK/ILC analysis, Applicants observed a gradient of naïve T cells to the left, and two paths leading to helper T cells and ILCs along the top axis, and cytotoxic T cells and NK cells on the bottom axis (FIG. 7). The three termini from top to bottom were ILCs, CD8+ CTLs, and NK cells. Between these two upper and lower paths, sat proliferating cells, which were present in lower frequencies in FGID patients and increased in frequency particularly in pediCD FR/PR patients (FIG. 7). Applicants quantified the overall differences in the distribution of FGID, NOA, FR and PR identifying significant differences in every comparison. As Applicants had noted significant differences within pediCD in proliferating T cells and NK cells, and this was maintained relative to FGID, Applicants identified gene expression signatures within the proliferative cells (FIG. 7).

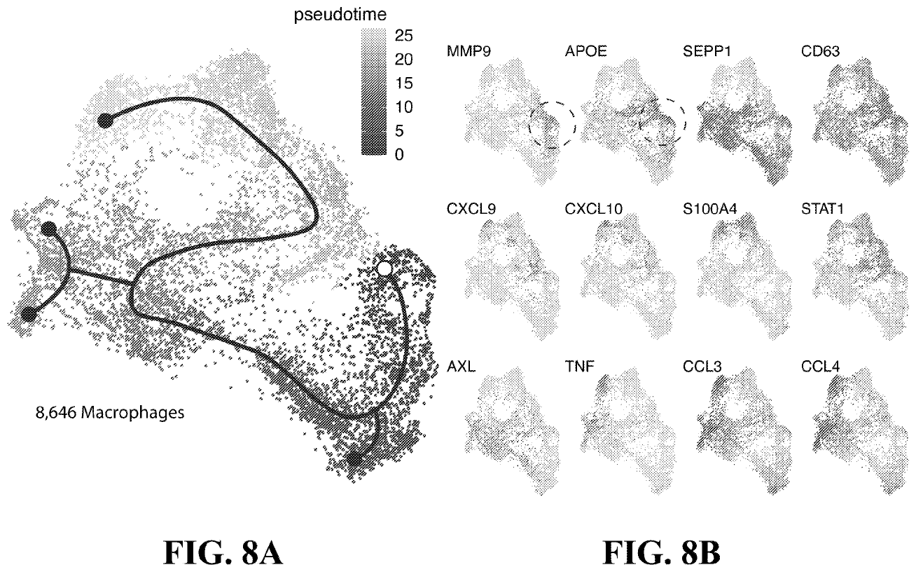

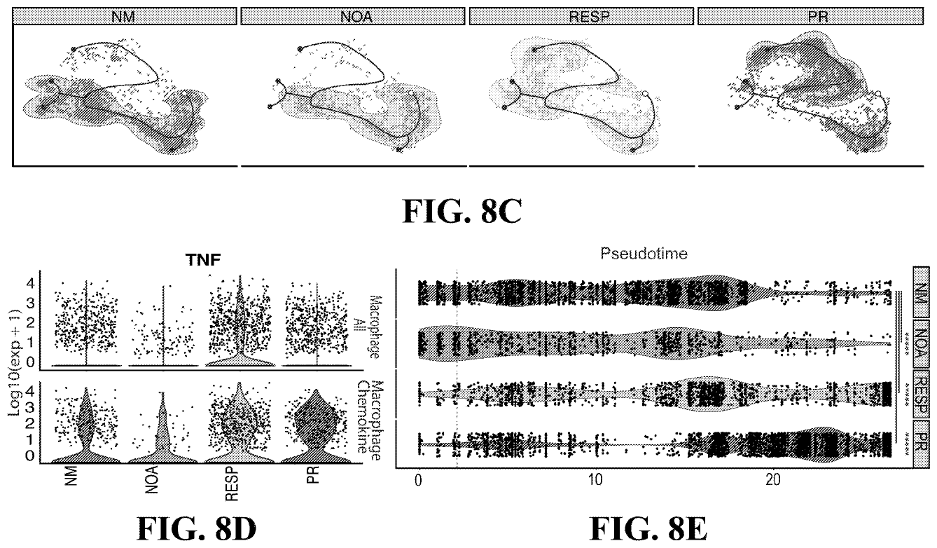

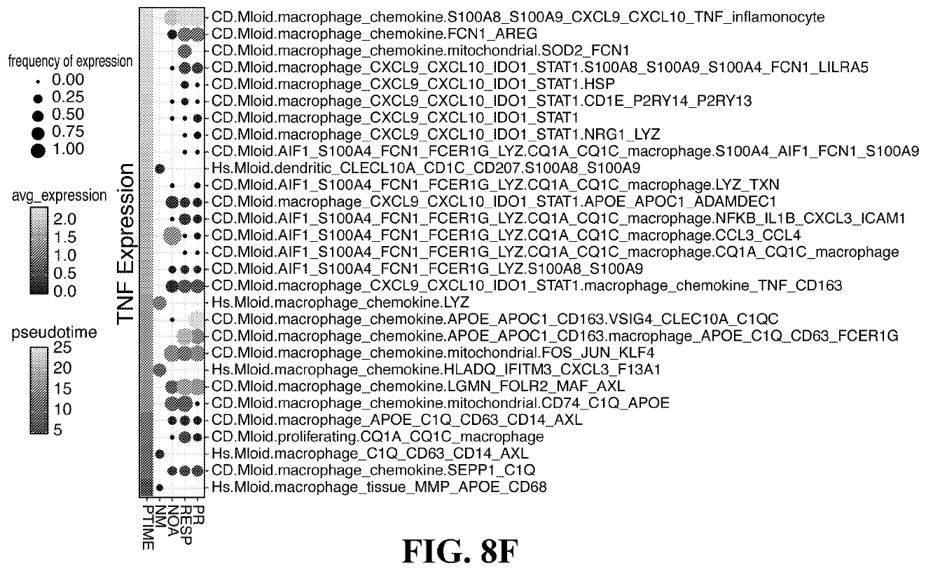

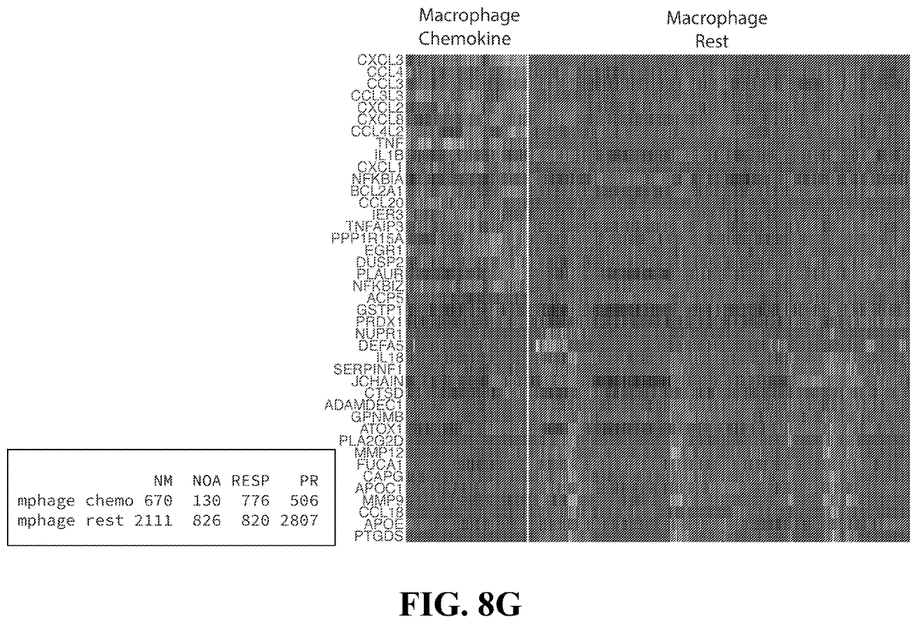

Within monocytes and macrophages, Applicants identified a gradient from right to left, with macrophages having a more homeostatic gene expression signature (MMP9 APOE) as the origin (FIG. 8). This path then led to a lower trajectory and an upper trajectory, whereby the lower as populated by cells from FGID, NOA, and FRs. PRs had fewer cells on this lower trajectory, with an increase in the upper arc, particularly in STAT1/S100A4 clusters, illustrating that this is unique to more severe forms of pediCD, and not seen in FGID. Comparing the pseudotime distribution between FGID and pediCD again showed significant differences in all comparisons. Together, the approach provides the ability to individually analyze the extent of cellular heterogeneity present in FGID or pediCD, resolving clusters absent the influence of other tissue states, then permits for quantifying the certainty and directionality of correspondence between cohorts, and project into a joint space to capture differences in distribution.

### Example 7—a Treatment-Naïve Cellular Atlas of Pediatric Crohn's Disease Predicts Disease Severity and Therapeutic Response

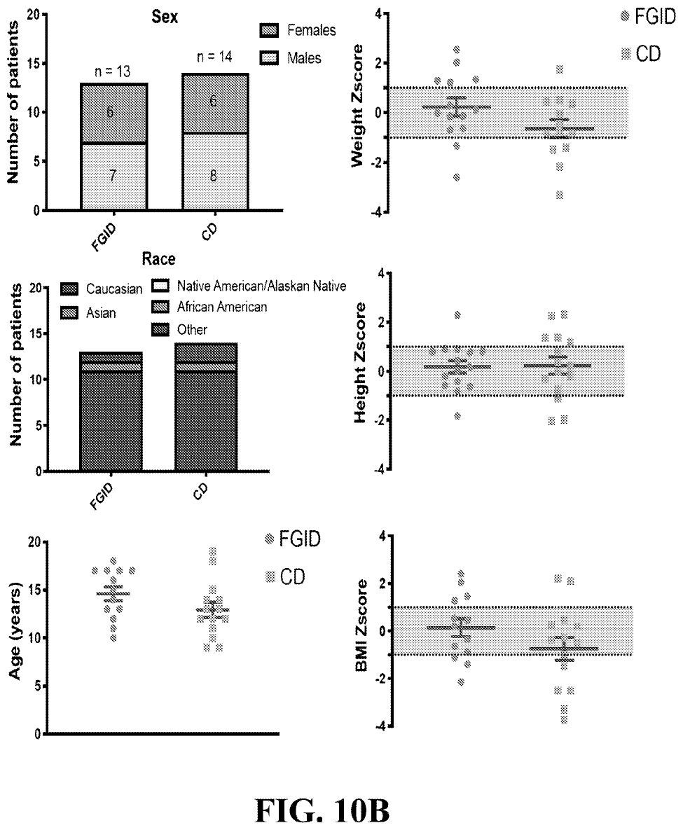

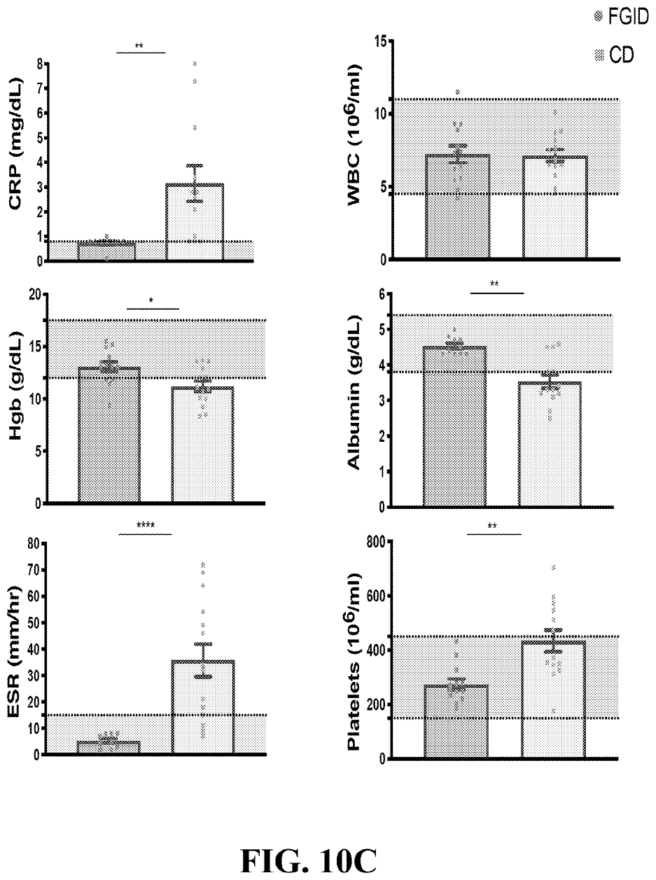

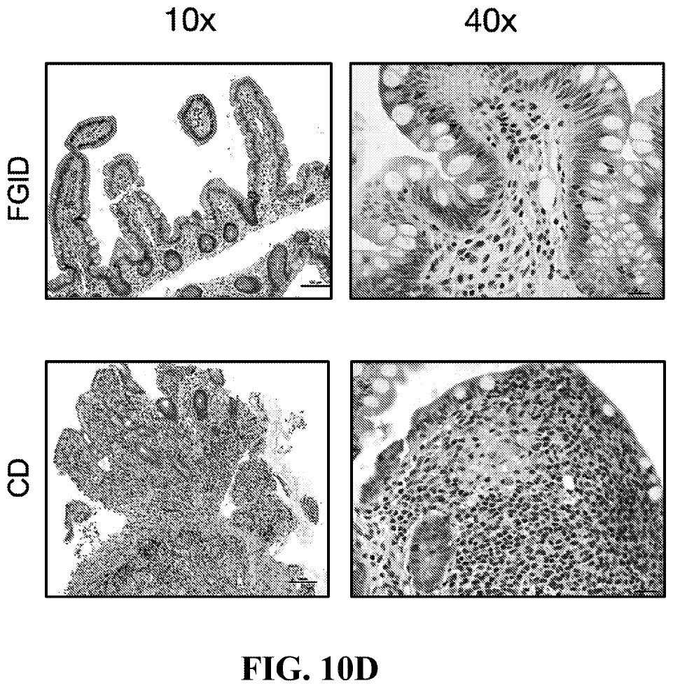

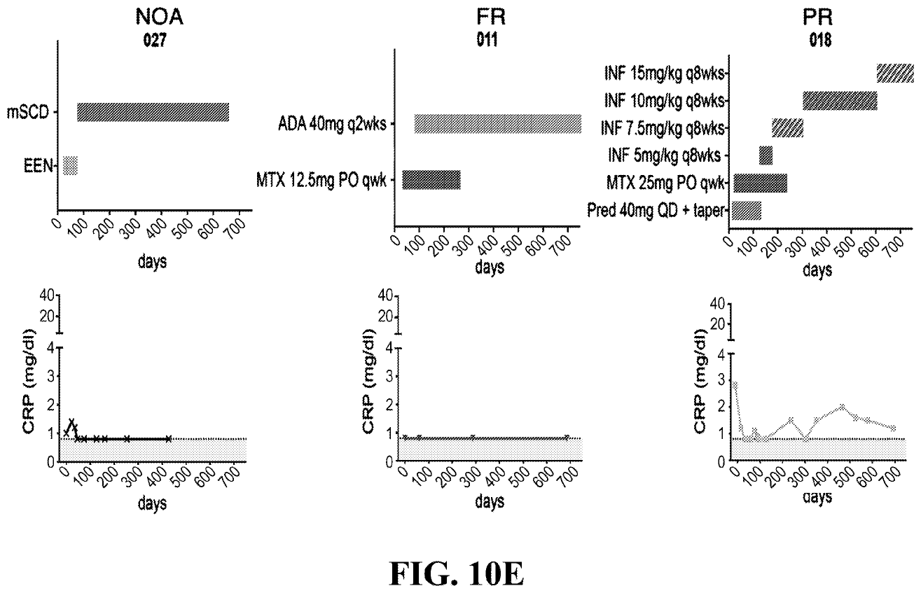

Most comprehensive scRNA-seq atlases of inflammatory disease conditions consist of patients being treated with a variety of agents, and for which the biopsies included often reflect a partial treatment-refractory state to combinations of antibiotics, corticosteroids, immunomodulators, and biologics including anti-TNF monoclonal antibodies. A treatment-naïve single-cell atlas in an inflammatory disease condition linking observed baseline cell clusters with disease trajectory and treatment outcomes has yet to be reported. In order to address this unmet need in pediCD, Applicants created the prospective PREDICT study (Clinicaltrials.gov #NCT03369353) to help identify, profile, and understand pediatric IBD and FGID controls. Here, Applicants present detailed diagnostic data from the first cohort of 27 patients enrolled on PREDICT, including 14 pediCD and 13 FGID patients, together with flow cytometric and scRNA-seq studies of the cellular composition of the terminal ileum (FIG. 10). Furthermore, through thorough, prospective annotation of clinical metadata and detailed longitudinal follow-up, Applicants stratify the pediCD cohort by clinically-guided therapeutic decisions separating patients treated with anti-TNF mAbs versus those with biopsy-proven pediCD, but for whom clinical symptoms were sufficiently mild that the treating physician did not prescribe anti-TNF agents (this cohort is termed “Not On Anti-TNF” or “NOA”). Importantly, Applicants were also able to separate the cohort of patients treated with anti-TNF agents into a sub-cohort of those who achieved a full response (FR) to this therapy, versus those who achieved only a partial response (PR). Critically, because PREDICT enrolled patients prior to their diagnostic endoscopy, Applicants were able to relate these clinical outcomes to the patients' cell states at diagnosis. Applicants contextualize the findings in pediCD relative to a cohort of 13 FGID patients, which provides an age-matched comparator cohort with clinical GI symptoms, but non-inflammatory disease proven by endoscopy and histologic examination.

Several analytical approaches have been developed to enable the generation and interrogation of clusters during the curation of single-cell transcriptomic atlases (Hie et al., 2020). One such method, sub-clustering of broad clusters, has proven to be a powerful tool for isolating highly specific axes of variation that are obscured by analyses whose principal axes of variation are broad cell types (La Manno et al., 2021; Tasic et al., 2018; Zeisel et al., 2018). However, while sub-clustering analysis is a powerful tool allowing access to the hierarchy of cell states, this method is manually intensive and there is little consensus, control, or standard in clustering parameters or annotation methods. To address this issue, Applicants developed a principled, modular, automated sub-clustering routine made possible by application of parameter scanning methods (Rousseeuw, 1987; Shekhar et al., 2016). Applicants developed this tool, ARBOL, of which iterative tiered clustering (ITC) is a key component, in R, integrating with Seurat functions, to make it accessible and easily incorporated into common workflows and have curated a GitHub repository with illustrative vignettes. Here, Applicants use ARBOL to standardize fine-grained cell state discovery by the creation and cultivation of a tree of cell states, followed by the generation of automated cell names to aid in the annotation of end clusters by unique and descriptive genes.

Together Applicants present two cellular atlases for pediatric GI disease, consisting of 94,451 cells for FGID and 107,432 for pediCD. Applicants provide key gene-list resources for further studies, identify correspondence between disease states, and nominate a vector of lymphoid, myeloid and epithelial cell states which predicts disease severity and treatment outcomes. This cellular vector correlates strongly with both the clinical presentation of pediCD severity, and to the distinction between anti-TNF full or partial response. The significant changes in cell composition associated with disease severity were increases of proliferating T cells, cytotoxic NK cells, specific monocytes/macrophages, and plasmacytoid dendritic cells (pDCs) accompanied by decreases of metabolically-specialized epithelial cell subsets. Applicants further validate this vector in two bulk RNA-seq treatment-naïve IBD cohorts.

### Example 8—Study Cohort Outcomes

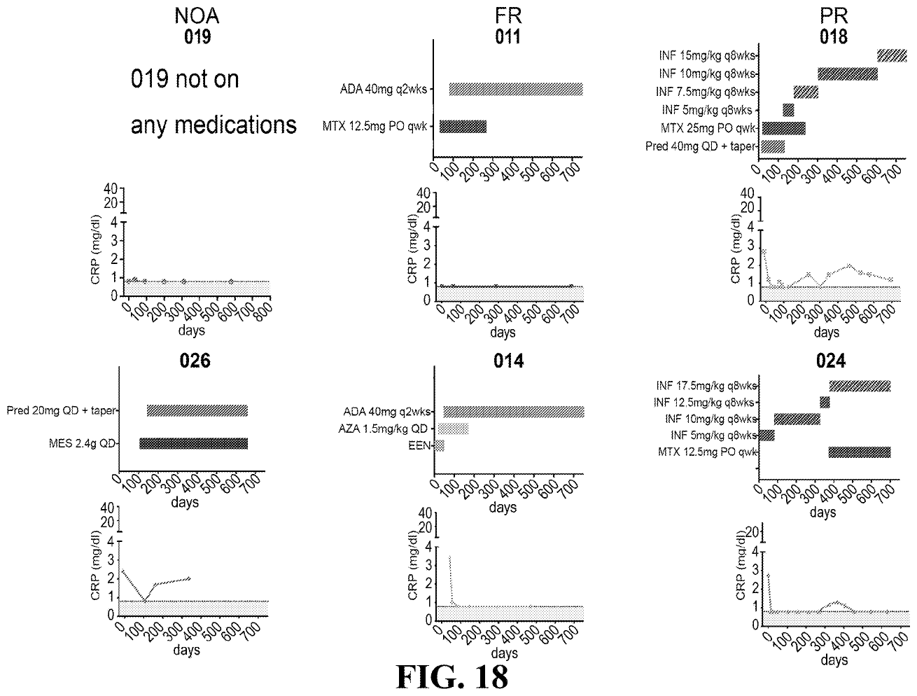

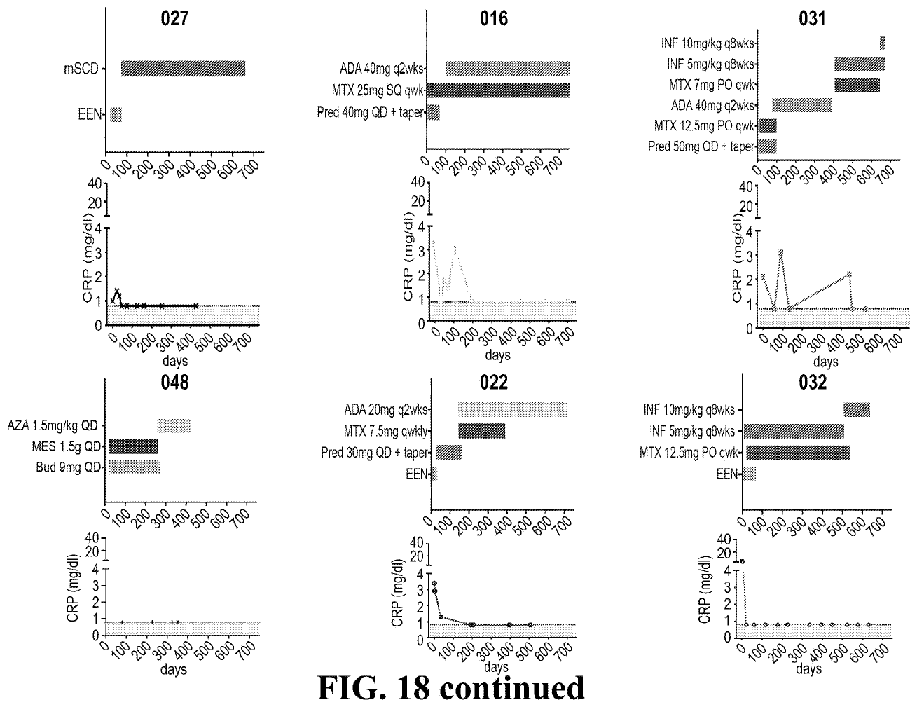

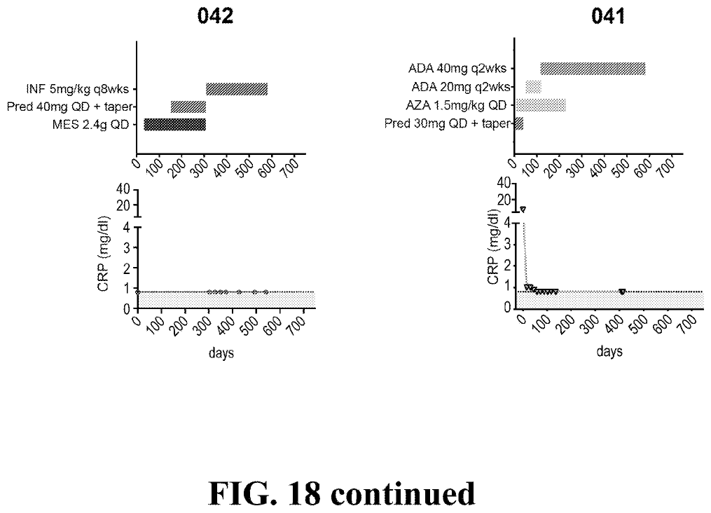

The PREDICT study prospectively enrolled treatment-naïve, previously undiagnosed pediatric patients with GI complaints necessitating diagnostic endoscopy. The current analysis focuses on patients enrolled in the first year of the study, during which time 14 patients with pediCD and 13 patients with FGID were enrolled and had adequate ileal samples for single cell analysis (FIG. 10; FIG. 18). Following their initial diagnosis, patients with pediCD were followed clinically for up to 3 years. Patients with FGID were followed up as needed in subspecialty/GI clinic. The median time from diagnosis for the pediCD and FGID cohorts as of Dec. 1, 2020 (time of database lock) was 32.5 and 31 months, respectively. Of the pediCD and FGID patients analyzed, the median age at diagnosis was 12.5 years and 16 years respectively (p=0.095), with no significant differences in gender (FIG. 10b; Table 5). Patient weight, height, and BMI z-scores were not significantly different between pediCD and FGID (FIG. 10b; Table 5); however, in addition to the diagnostic differences on histologic analysis, several key clinical laboratory values, including C-reactive protein (CRP), Erythrocyte Sedimentation Rate (ESR), hemoglobin concentration, albumin concentration, and platelet count were significantly different between pediCD and FGID (FIG. 10c,d; Table 5).

### Example 9—Treatment with Anti-TNF Agents and Response to Therapy

Patients with pediCD were initially divided into two cohorts. Those with milder disease characteristics (n=4) as determined by their treating physician, were not put on anti-TNF therapies, and are noted as NOA. For patients with more severe disease (n=10), anti-TNF therapy (with either infliximab or adalimumab, Table 6) was initiated within 90 days of diagnostic endoscopy. All pediCD patients were followed prospectively and categorized as FR (n=5) or PR (n=5) to anti-TNF therapy based on the following criteria: FR was defined as clinical symptom control and biochemical response (measuring CRP, ESR, albumin, and complete blood counts (CBC)), and with a weighted Pediatric Crohn's Disease Activity Index (PCDAI) score of <12.5 on maintenance anti-TNF therapy with no dose adjustments required (Cappello and Morreale, 2016; Hyams et al., 1991; Sandborn, 2014; Turner et al., 2012, 2017). PR to anti-TNF therapy was defined as a lack of full clinical symptom control as determined by the treating physician or lack of full biochemical response, with documented escalation of anti-TNF therapy or addition of other agents (FIG. 10e; NB: patients in the cohort were dose escalated because of clinical symptoms). Medication timelines and clinical laboratory data through 2 years of follow-up for all pediCD patients is shown in FIG. 18. The designation of FR or PR was made at 2 years of follow-up for all pediCD patients.

### Example 10—Flow Cytometry of the Terminal Ileum Reveals Minimal Changes in Leukocyte Subsets in FGID Vs. pediCD, and No Significant Differences Across the pediCD Spectrum

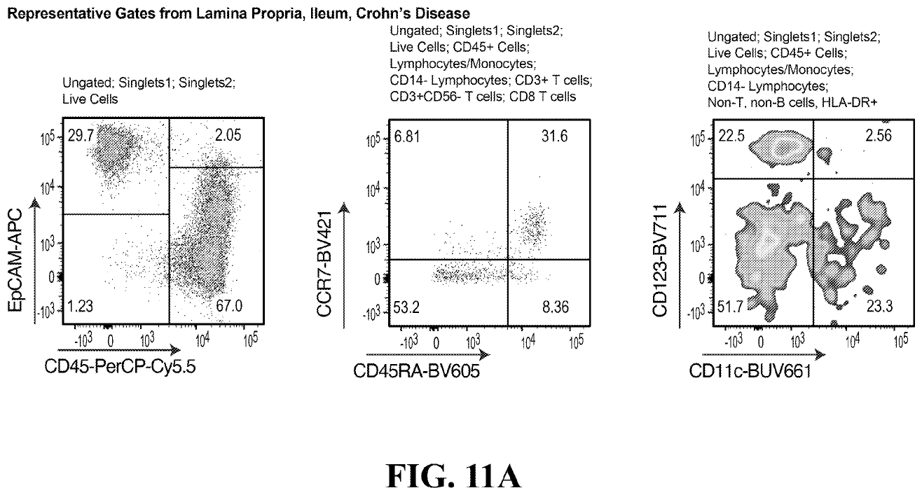

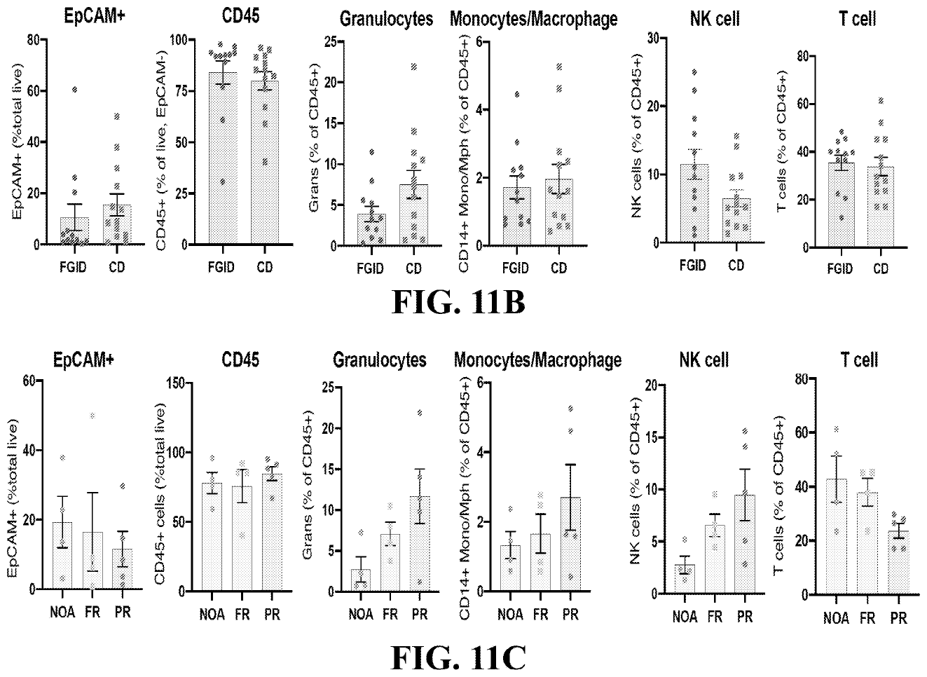

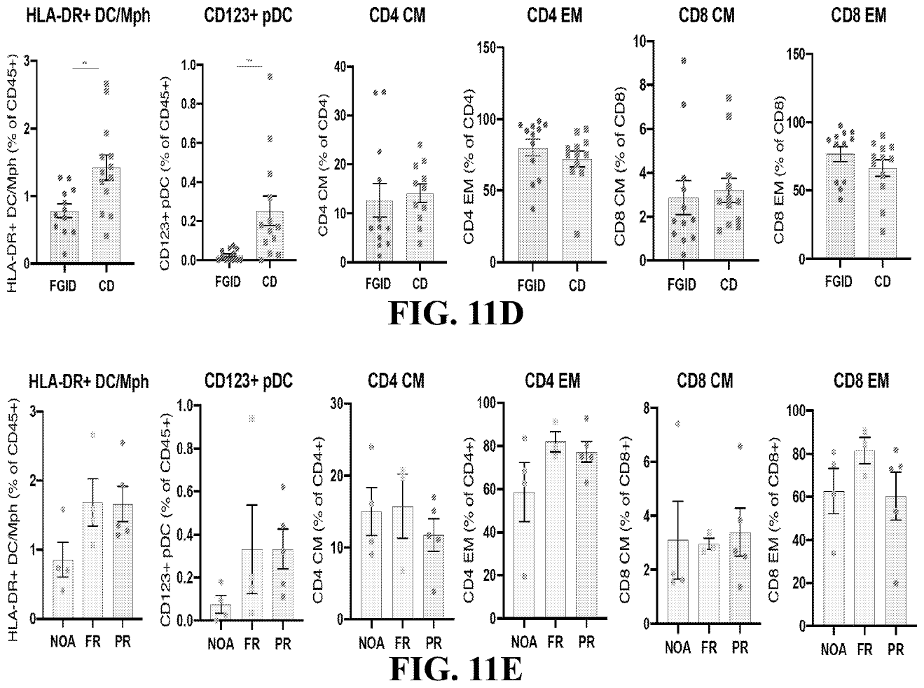

Applicants collected terminal ileum biopsies from 14 pediCD patients and from 13 uninflamed FGID patients, and prepared single-cell suspensions for flow cytometry and scRNA-seq. Biopsies from pediCD were from actively-inflamed areas adjacent to ulcerations. Biopsies from FGID were from non-inflamed terminal ileum. The epithelium was first separated from the lamina propria before enzymatic dissociation, and flow cytometric analysis was performed on the viable single-cell fraction, which recovered predominantly hematopoietic cells with some remnant epithelial cells (<20% of all cells), likely representing those in deeper crypt regions (FIG. 11;

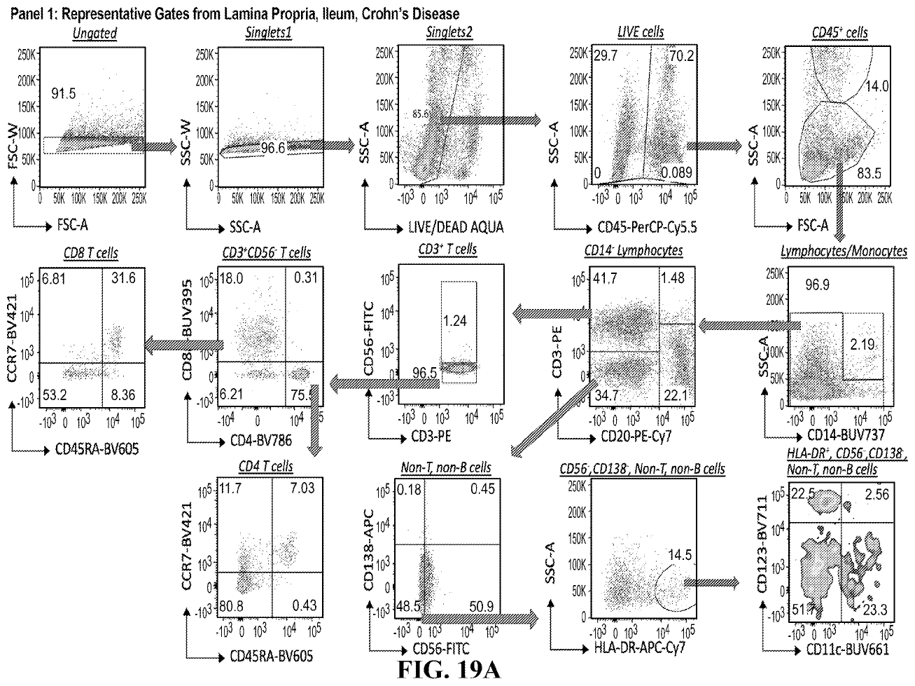

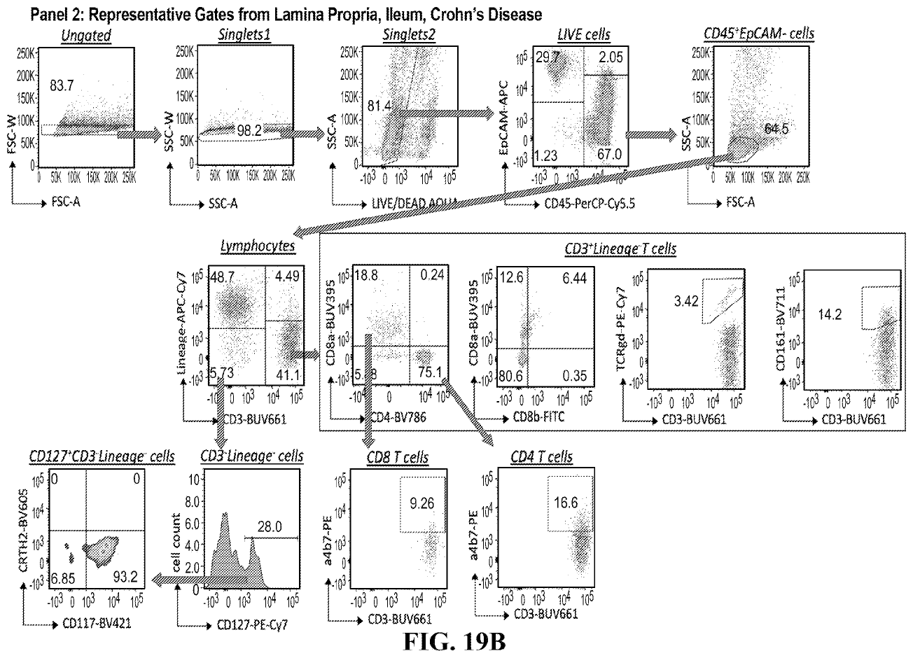

FIG. 19). Applicants utilized two flow cytometry panels, allowing Applicants to resolve the principal lymphoid (CD4 or CD8 T cells, NK cells, B cells, innate lymphoid cells, γδ T cells, CD8αα+ IELs, pDCs) and myeloid (monocytes, granulocytes, HLA-DR+ mononuclear phagocyte) cell subsets (FIG. 19, Table 7). From these panels, which generated 32 gates identifying cell lineages, types and subsets, only HLA-DR+ macrophages/DCs and pDCs were significantly increased in pediCD relative to FGID (Figure lid). Applicants also analyzed within pediCD, comparing the baseline samples of 4 NOA, 5 FR and 5 PR patients, and noted no significant differences between NOA and patients on anti-TNF, or between FRs and PRs to anti-TNF (Figure lie). Together, this suggests that despite the substantial endoscopic, histologic, and clinical parameters that distinguish FGID and pediCD (FIG. 10), the broad single-cell type composition of the terminal ileum appears minimally altered in pediCD save for an increased frequency of pDCs and HLA-DR+ mononuclear phagocytes.

### Example 11—Traditional Joint scRNA-Seq Clustering of FGID and pediCD Patients

In addition to flow cytometry, Applicants performed droplet-based scRNA-seq on cell suspensions from the 14 pediCD/13 FGID patient cohort using the 10× Genomics V2 3′ platform (FIG. 10). The analyzed cell suspensions were derived from lamina propria preparations, which the flow cytometry data suggested would be composed primarily of CD45+ leukocytes, alongside a small fraction of epithelial cells and stromal/vascular cells. Deconstructing these tissues into their component cells provided Applicants with the ability to identify some of the corresponding cell types (e.g. T or B cell) and subsets (CD8αα+ IEL or CD4+ T cell) to those Applicants identified by flow cytometry data but importantly also enabled Applicants to: 1. characterize these major cell types and subsets without needing to pre-select markers, and 2. gain substantially enhanced resolution into the cell states (i.e. gene expression programs) within these types and subsets.

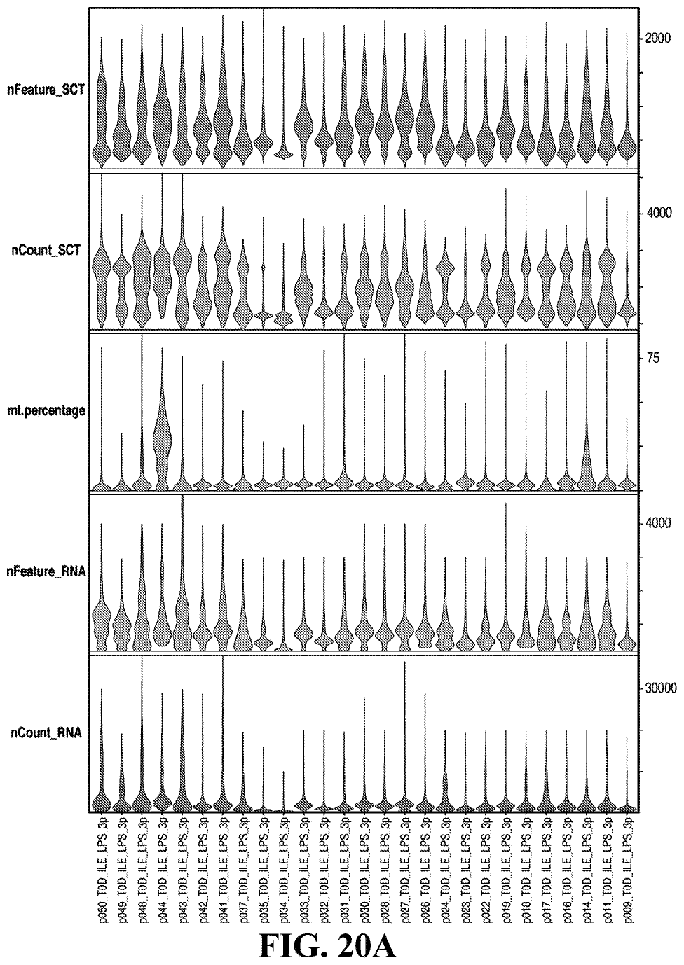

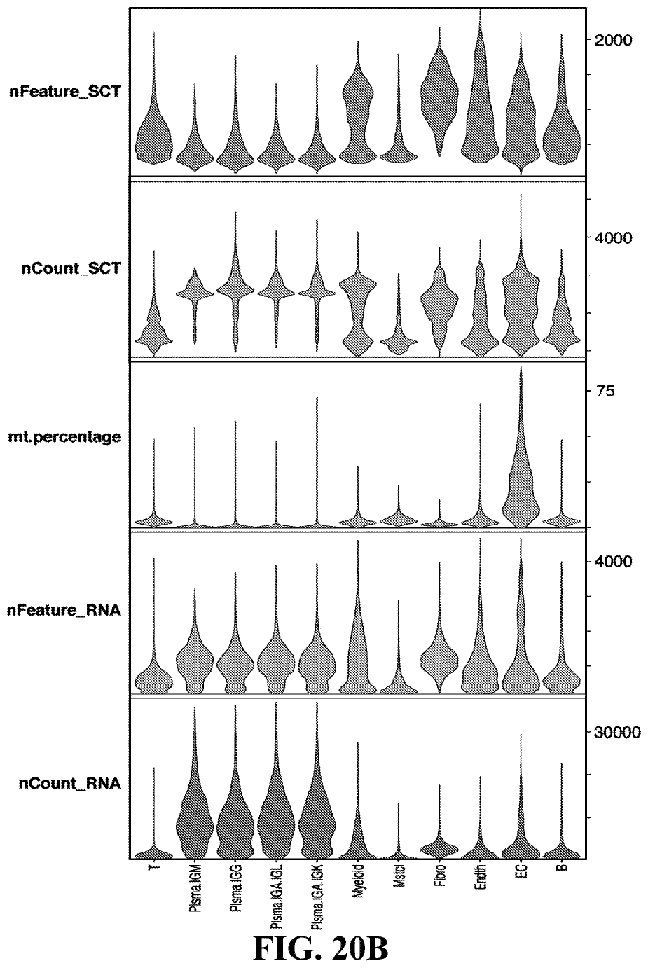

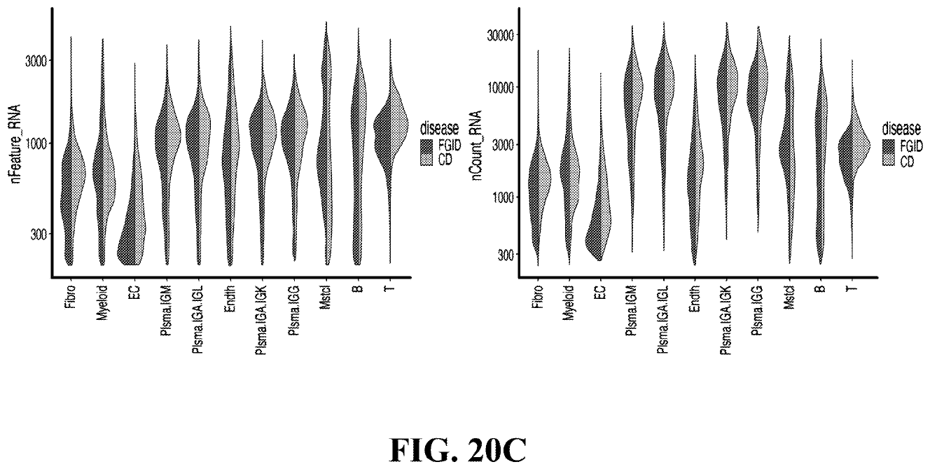

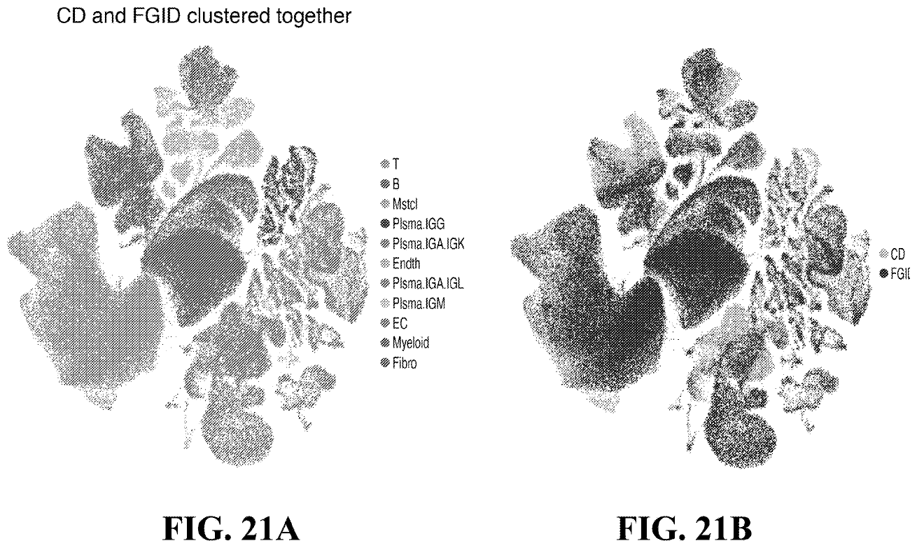

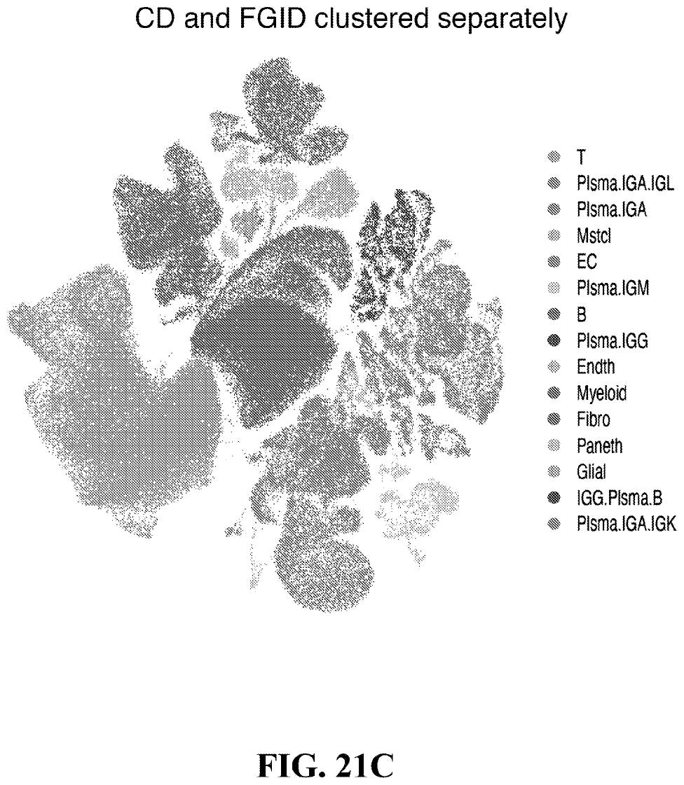

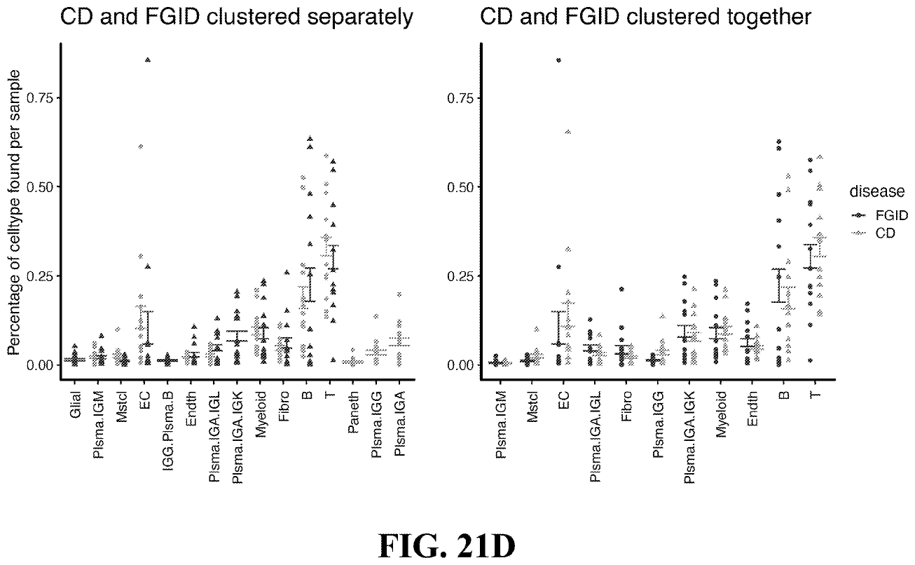

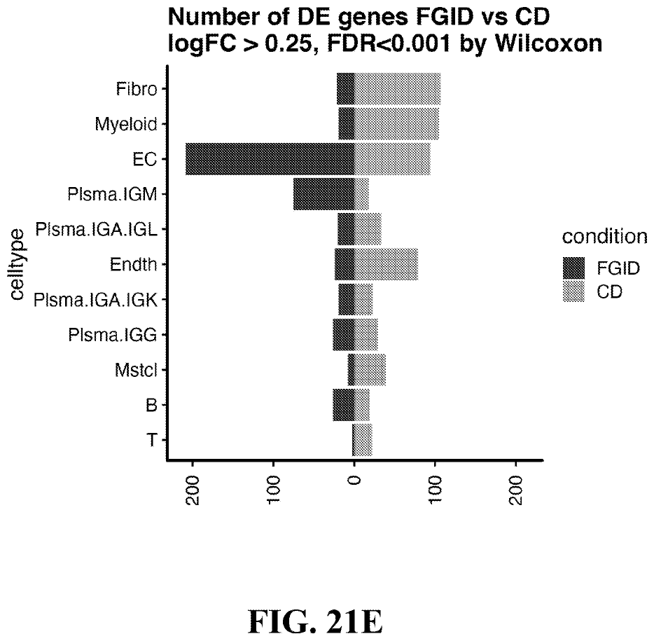

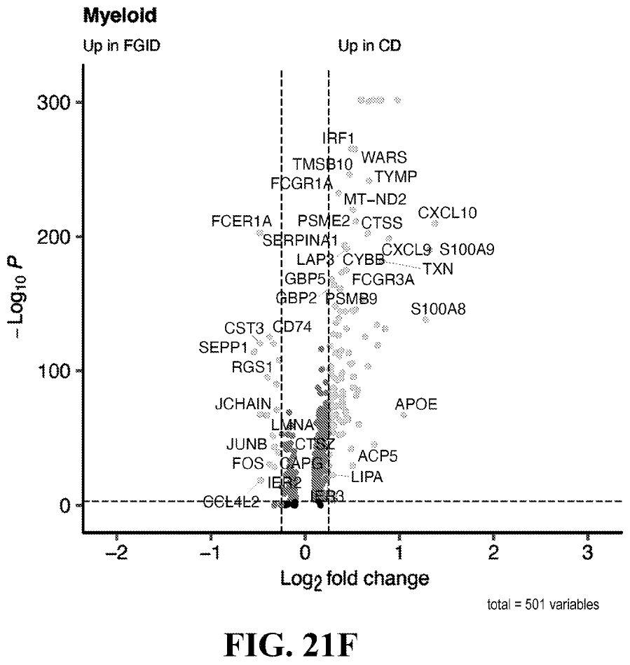

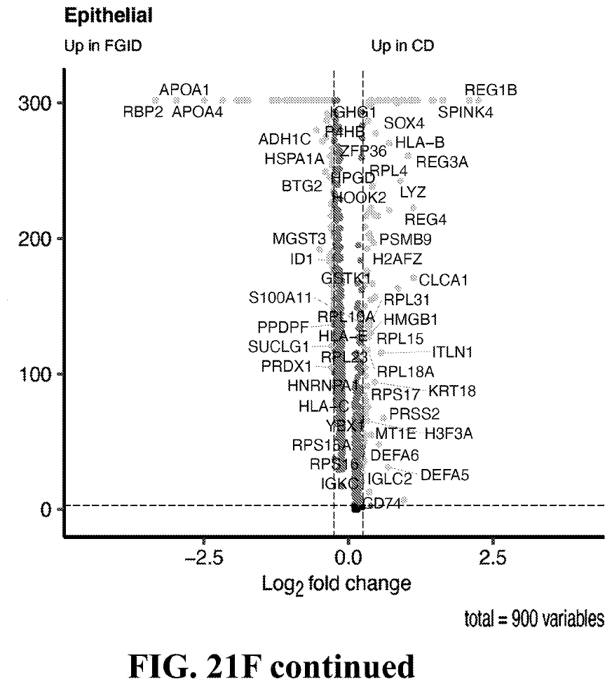

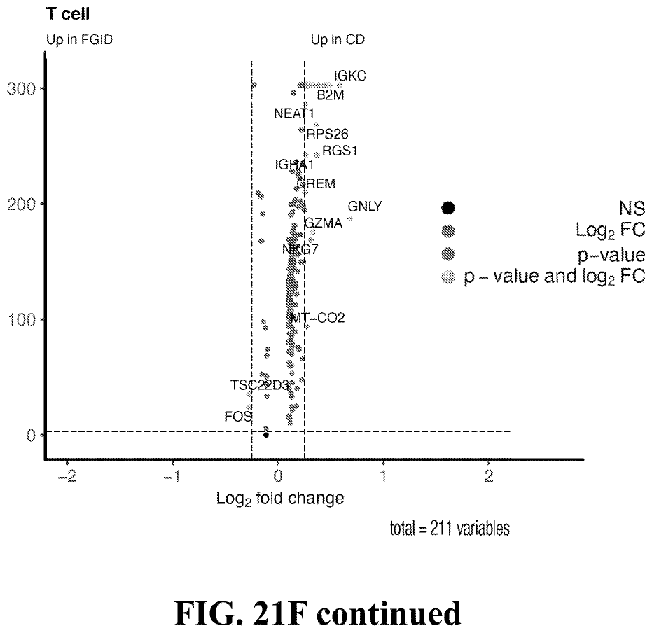

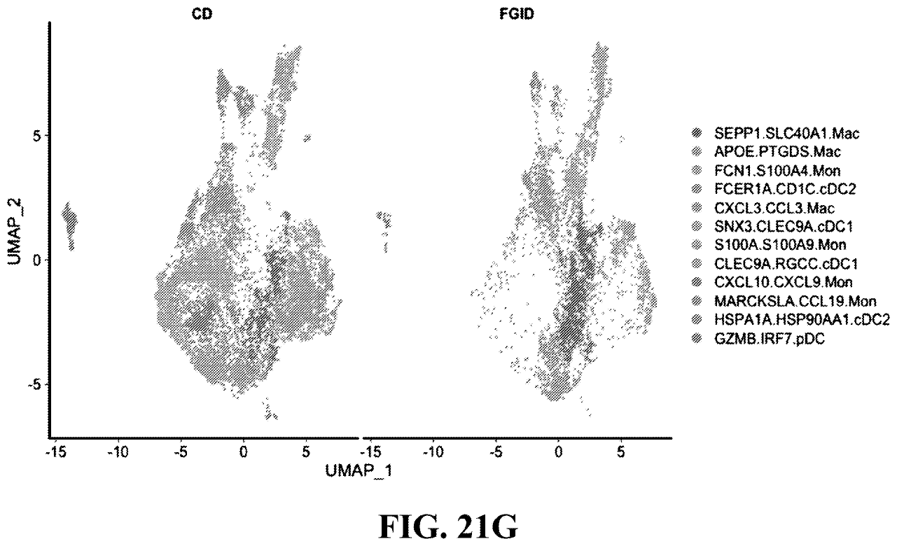

Following library preparation and sequencing, Applicants derived a unified cells-by-genes expression matrix from the 27 samples, containing digital gene expression values for all cells passing quality thresholds (n=254,911 cells; FIG. 20; Methods). Applicants then performed dimensionality reduction and graph-based clustering, noting that despite no computational integration methods being used, FGID and pediCD were highly similar to each other when visualized on a uniform manifold approximation and projection (UMAP) plot (FIG. 21a-c). Applicants recovered the following cell types from both patient groups: epithelial cells, T cells, B cells, plasma cells, glial cells, endothelial cells, myeloid cells, mast cells, fibroblasts, and a proliferating cell cluster. Applicants noted that the fractional composition amongst all cells of T cells, B cells, and myeloid cells was not significantly different between FGID and pediCD, similar to the flow cytometric data, and this was also the case for endothelial, fibroblasts, glial, mast and plasma cells, which were not measured through flow cytometry (FIG. 21d). This provided validation and extension of the flow cytometry data documenting that the broad cell type composition of FGID and pediCD is not significantly altered, despite highly distinct endoscopic and histologic diseases. Based on this joint clustering and annotation of top-level cell types, Applicants then performed differential expression testing identifying significant up- and down-regulated genes across all cell types (FIG. 21e). Within myeloid cells Applicants identified some of the most significantly upregulated genes in pediCD versus FGID to be CXCL9 and CXCL10, canonical IFNγ-stimulated genes, and S100A8 and S100A9 which form the biomarker fecal calprotectin (FIG. 21f)(Leach et al., 2007; Ziegler et al., 2021). Within epithelial cells Applicants identified that APOA1 and APOA4 were amongst the most significantly downregulated genes, and correspondingly REG1B, SPINK4 and REG4A were amongst the most significantly upregulated indicating tradeoffs between lipid metabolism and host defense (FIG. 21f) (Haberman et al., 2014). In T cells, Applicants noted that the cytotoxic genes GNLY and GZMA were amongst the most significantly upregulated, with almost no genes downregulated (FIG. 21f).

Applicants then systematically re-clustered each broad cell type, identifying increasing cellular heterogeneity. Given that Applicants detected changes in the frequency of HLA-DR+ macrophages/dendritic cells and pDCs between pediCD and FGID by flow cytometry, Applicants initially focused on the myeloid cell type sub-clustering, containing dendritic cells, macrophages, monocytes, and pDCs (FIG. 21g). Working within this analysis paradigm revealed that a traditional clustering approach had difficulty identifying the boundaries of clusters, and whether a cluster composed primarily of pediCD rather than FGID cells represented a unique cell subset, or a cell state overlaid onto a core cell subset gene expression program (FIG. 21g, Methods). These distinctions would influence whether a comparison would primarily focus on differential expression testing or differential composition testing between the two clinical cohorts. It also raised the possibility that this joint clustering approach, informed by the inclusion of both FGID and pediCD cell types, subsets and states could muddle some of the unique biology of FGID and pediCD. For instance, this analytical approach could lead to hybrid clusters, informed by cells from both FGID and pediCD and correspondingly critical gene-reference lists for each cluster, that may not accurately represent disease-specific cell types, subsets, or states.

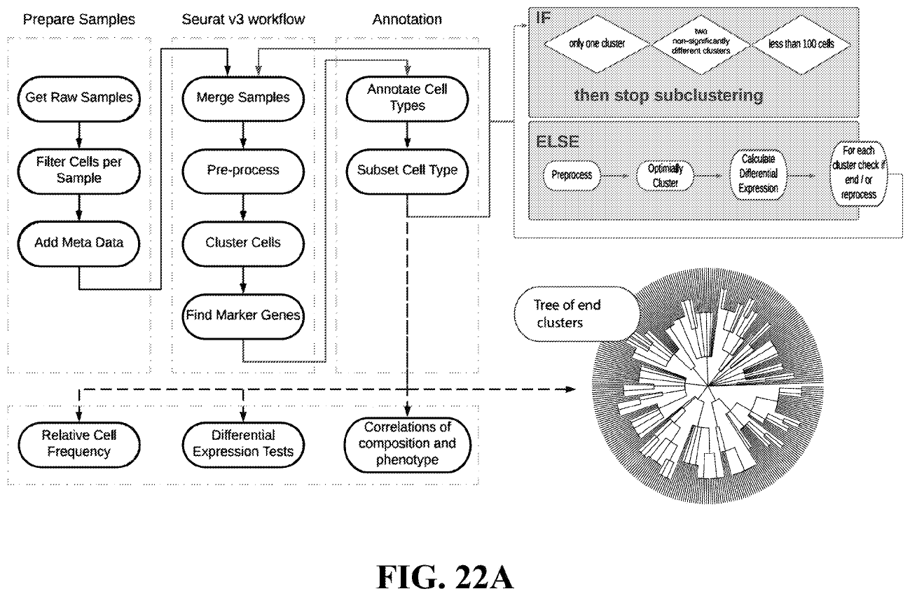

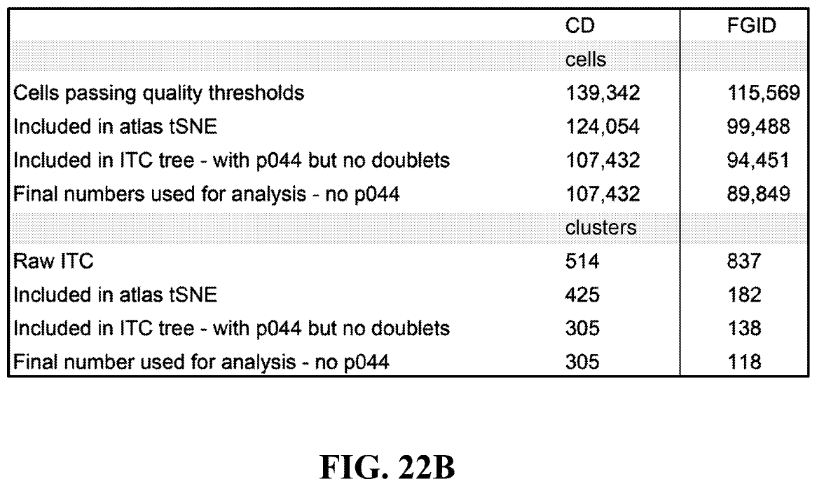

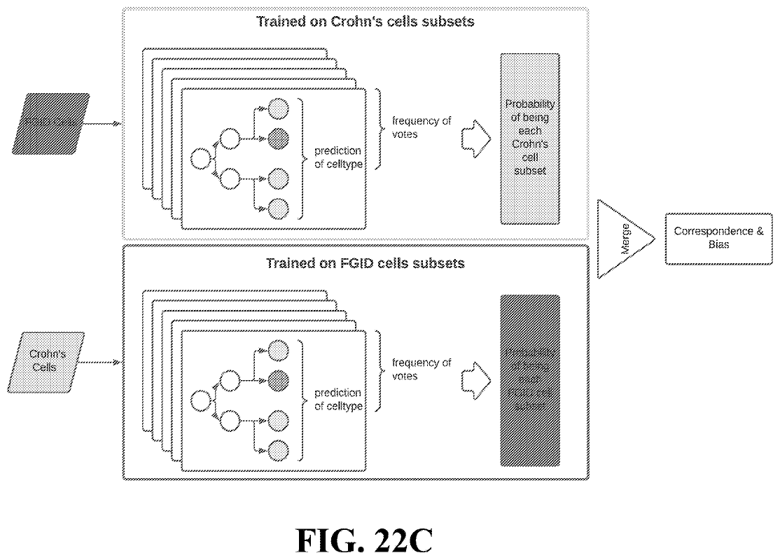

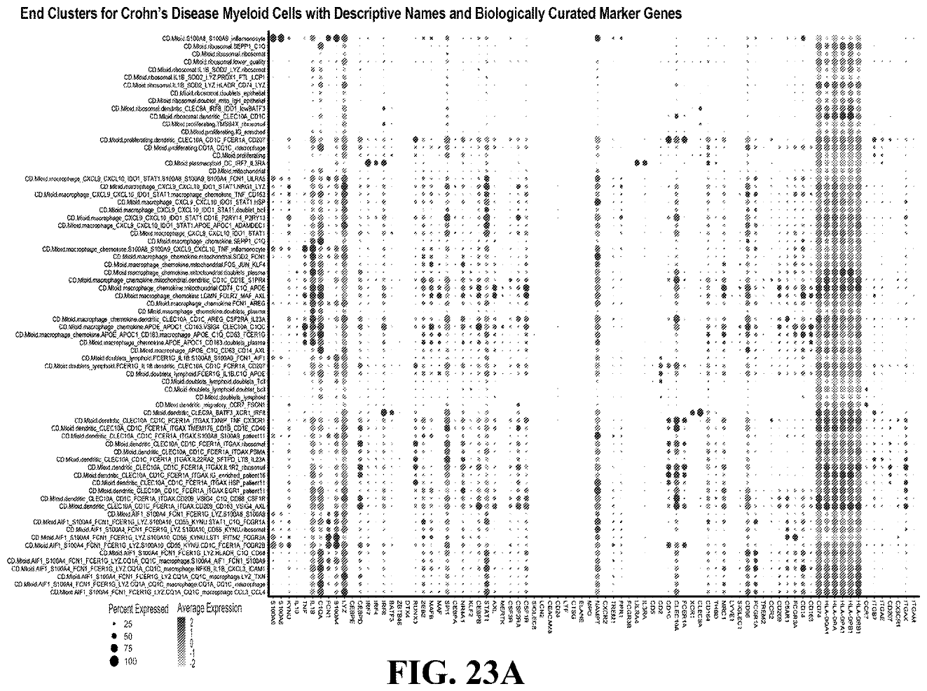

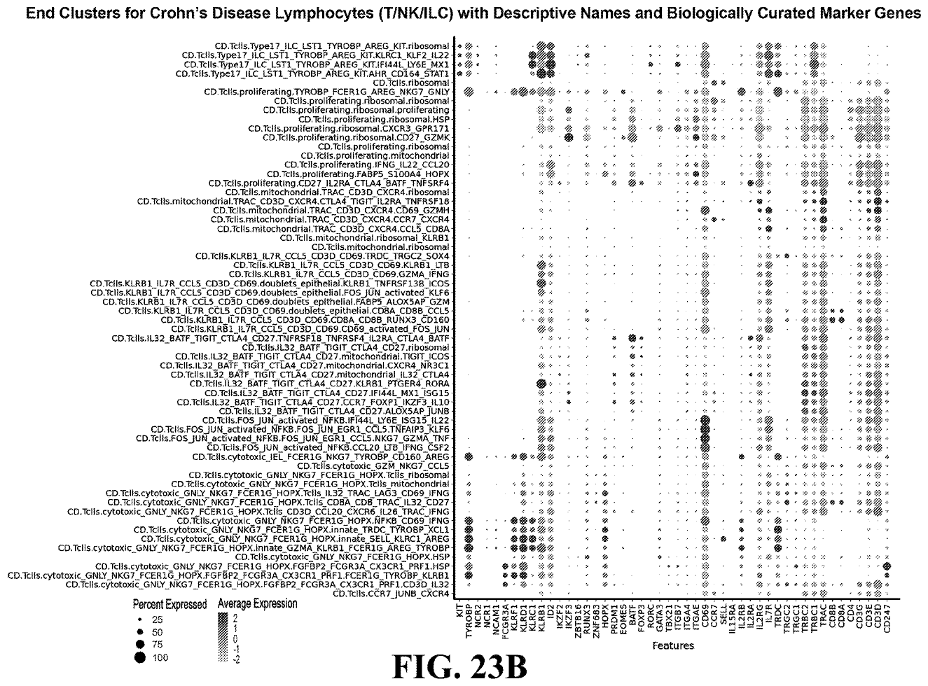

In order to approach this challenge from a more principled direction, Applicants made five key changes to the analytical workflow, which Applicants jointly refer to as ARBOL (github.com/jo-m-lab/ARBOL): 1. Applicants proceeded to analyze FGID and pediCD samples separately to define corresponding cell type, subset, and state clusters and markers, 2.Applicants implemented an automated ITC approach to optimize the silhouette score at each tier of iterative sub-clustering and stop when a specific granularity was reached (FIG. 22a,b; Methods), 3. Applicants systematically generated descriptive names for cell types and subsets together with differentiating marker genes, 4. Applicants accounted for the number and diversity of patients which compose each cluster using Simpson's Index of Diversity, and 5. Applicants generated and optimized a Random Forest classifier to identify correspondence between the resultant FGID and pediCD atlases (FIG. 22c; Methods) (Simpson, 1949). Using this approach, each tier of analysis is typically under-clustered relative to traditional empirical analyses, but the automation proceeds through several more tiers (typically 6 to 7) until stop conditions (e.g. cell numbers and differentially expressed genes; Methods) are met. Applicants then inspected all outputs (182 FGID and 425 pediCD clusters) and provided descriptive cell cluster names independently for FGID and pediCD (FIGS. 22b and 23). Applicants also focused at this stage on flagging putative doublet clusters or clusters where the majority of differentially expressed genes which triggered further clustering consist of known technical confounders in scRNA-seq data (e.g. mitochondrial, ribosomal, and spillover genes from cells with high secretory capacity) yielding a final number of 118 FGID and 305 pediCD clusters (FIG. 22b). Applicants note this clustering method represents a data-driven approach, though it may not always reflect a cellular program or transcriptional module of known biological significance.

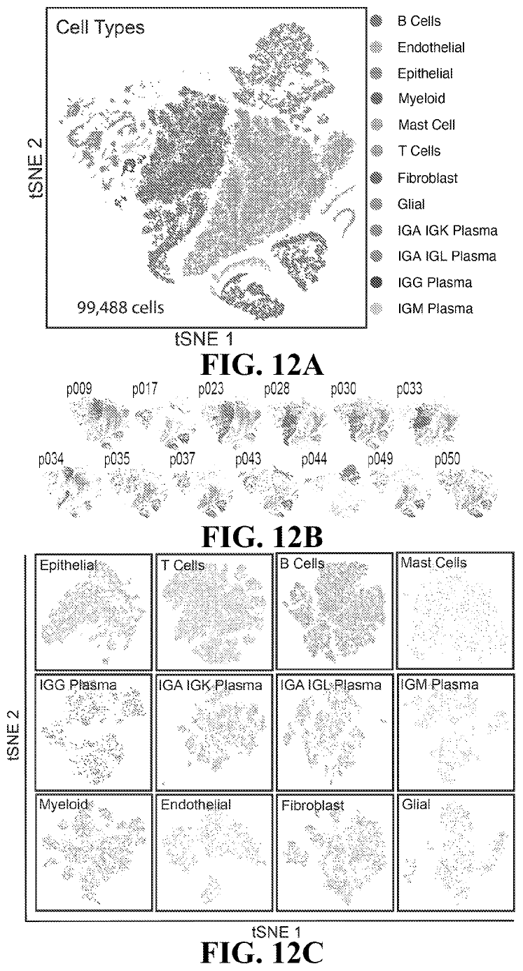

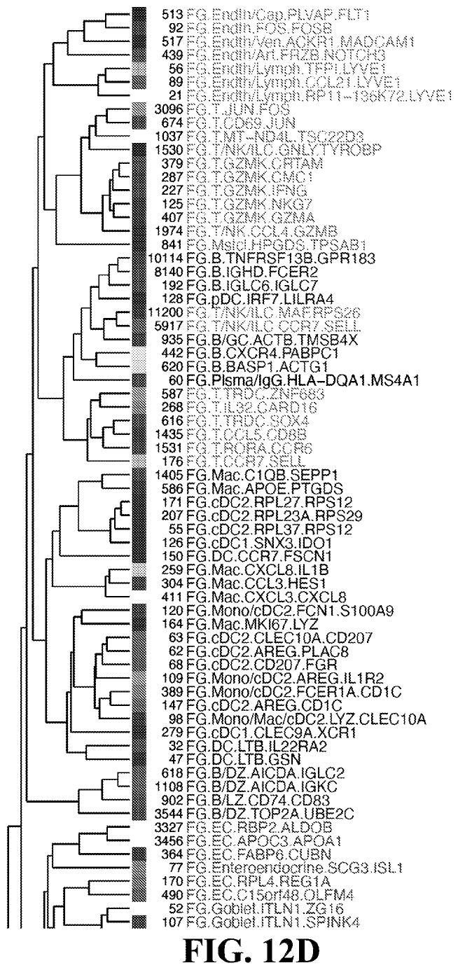

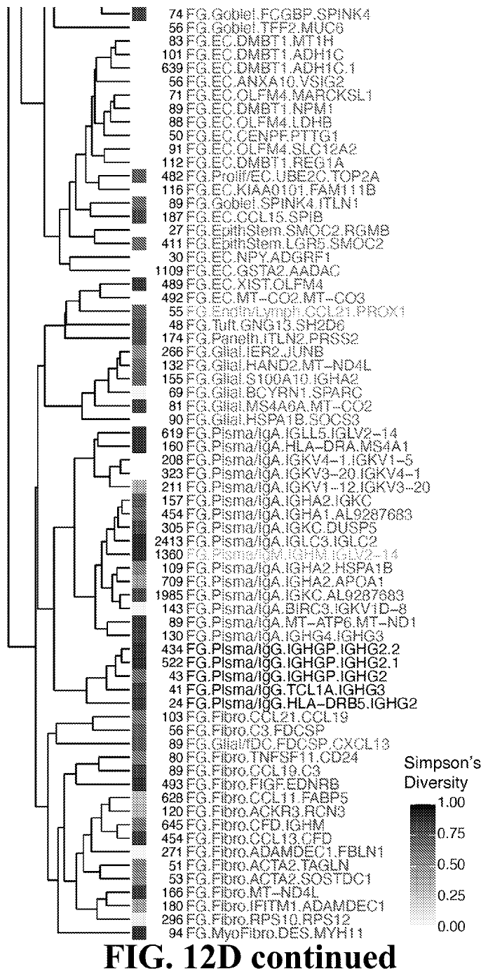

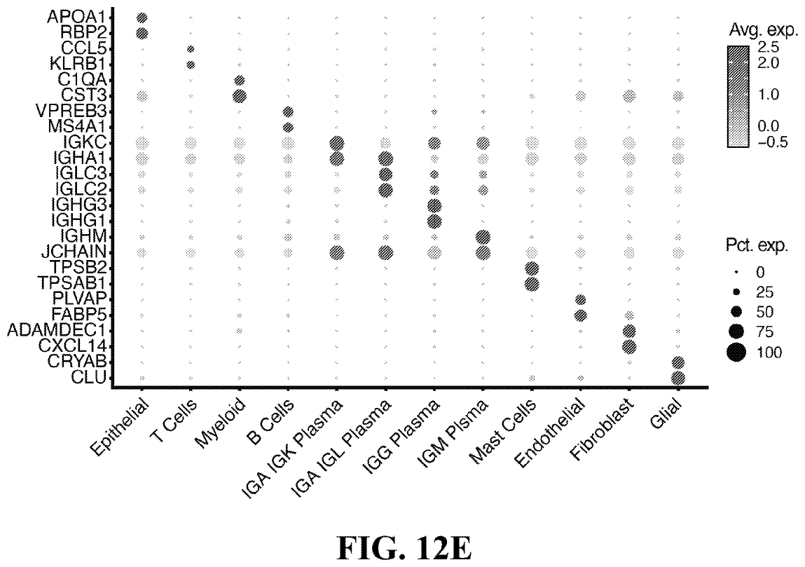

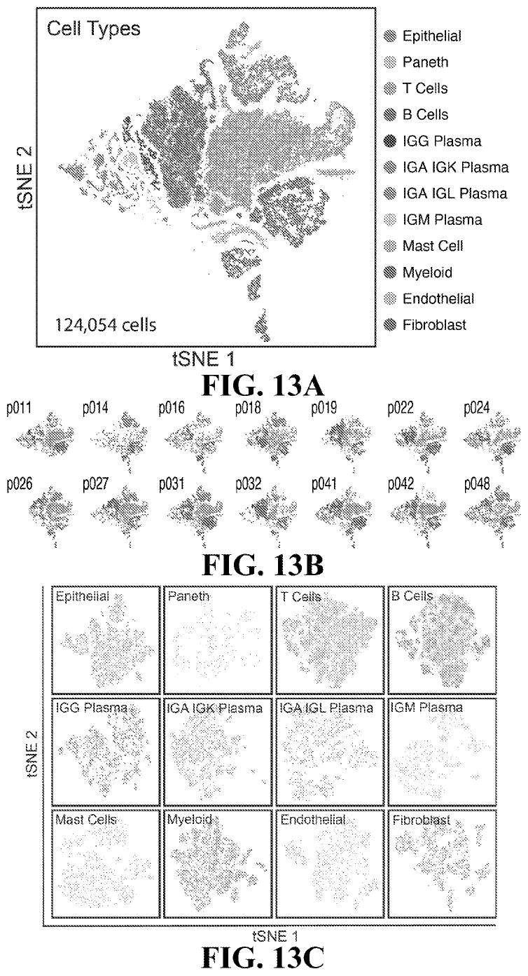

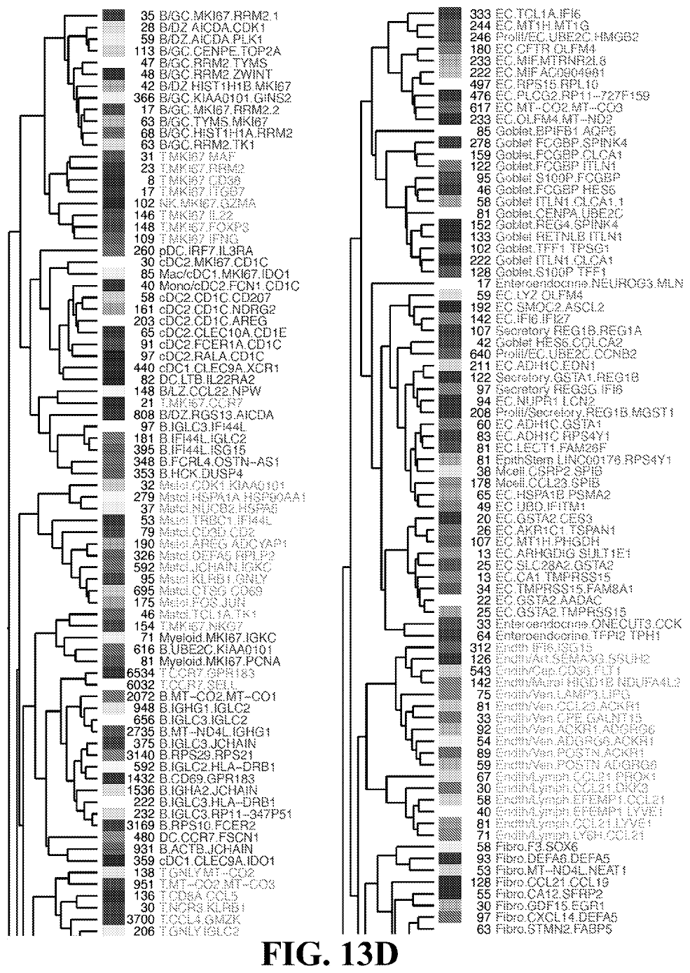

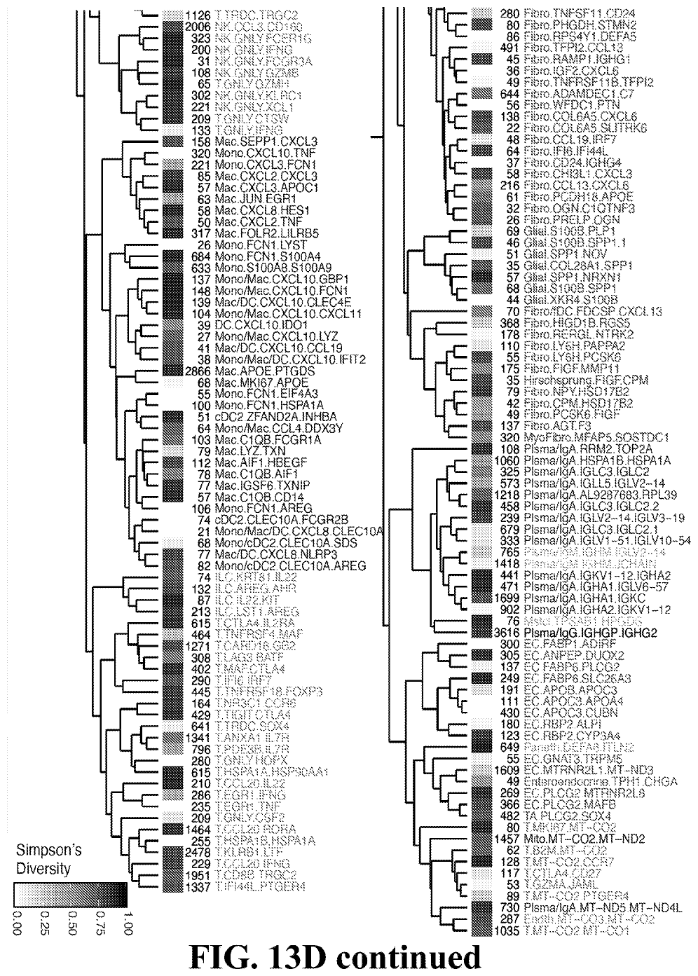

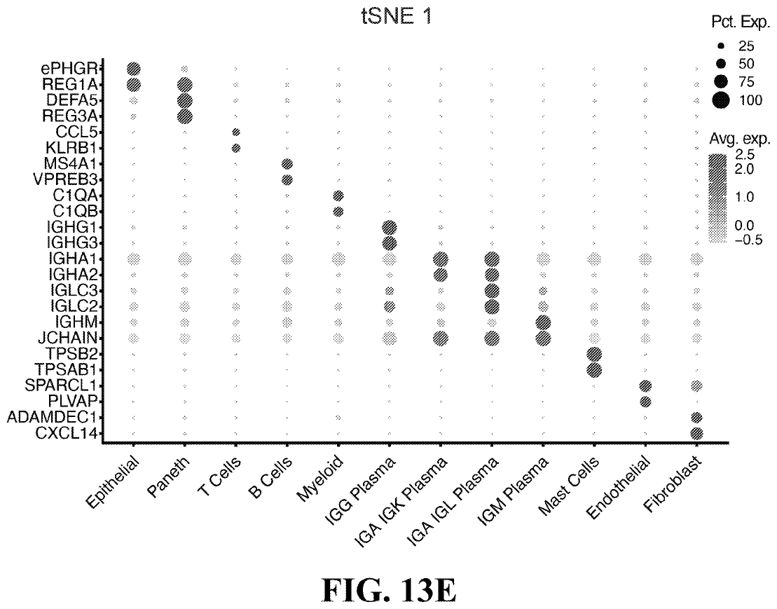

Applicants then hierarchically clustered all end cell state clusters to generate the final dendrograms for FGID and pediCD, and performed 1 vs. rest within-Tier 1 clusters (i.e. broad cell types) differential expression to provide systematic names for cells based on their cell type classification and two genes (FIGS. 12 and 13; Methods). As several cell types contained readily identifiable and meaningful cell subsets, Applicants utilized curation of literature-based markers to provide further guidance within each cell type (Bleriot et al., 2020; Cherrier et al., 2018; Dutertre et al., 2019; Guilliams et al., 2018; Robinette and Colonna, 2016). For example, within Tier 1 T cells, Applicants could identify T cells, NK cells and ILCs; within Tier 1 myeloid cells, monocytes, cDC1, cDC2, macrophages and pDCs; within Tier 1 B cells, germinal center, germinal center dark zone and light zone cells; within Tier 1 endothelial cells, arterioles, capillaries, lymphatics, mural cells and venules; and so forth for other cell types. To illustrate this process for one cluster, upon automated hierarchical tiered clustering of T cells, Applicants identified a cluster that was Tier 0: pediCD, Tier 1: T cells, Tier 2: cytotoxic, Tier 3: IEL_FCER1G_NKG7_TYROBP_CD160_AREG. Upon inspection of CD3 genes (CD247, CD3D, etc.), TCR genes (TRAC, TRBC1, etc.), and NK cell genes (NCAM1, NCR1), it became readily apparent these cells were NK cells (FIG. 23).

Applicants generated gene lists for cell types (1 vs. rest across all cells), subsets (1 v. rest across all cells), and states (1 vs. rest within-Tier 1 cell-type) (see, Table 1 and Table 4). To select marker genes for naming in a data driven manner Applicants used 1 vs. rest within-cell-type differential expression (Table 1 and Table 4; Wilcoxon, Bonferroni adjusted p<0.05). To account for genes that might be highly expressed in just a few cells Applicants ranked the marker genes by a score combining their significance, the fold change in expression, and fold change of percent gene positive cells in the subset versus the percent of gene positive cells outside the subset. The collected metrics were multiplied together to provide a single score by which the genes were ranked: (−log(sig+1)*avg_log FC*(pct.in/pct.out)). For most subsets Applicants selected the top 2 of these marker genes. For T/NK/ILC cells and myeloid cells Applicants occasionally chose a slightly lower ranking gene from the top 10 if it was well supported and recognized by the literature. Using this ranking system, Applicants identified CCL3 and CD160 as two genes significantly enriched in one NK cluster (adj. p-value=0, expression within-cluster >40% cells positive and in other Tier 1 T cells <6%). This resulted in a final name for this cluster of CD.NK.CCL3.CD160. Applicants repeated this process for all FGID (183 end clusters) and pediCD (426 end clusters) within Tier 1 B cell, endothelial, epithelial, fibroblast, plasma cell, myeloid cell, mast cell, and T cell identified clusters, and provide systematically generated names for all (Tables 8 and 9).

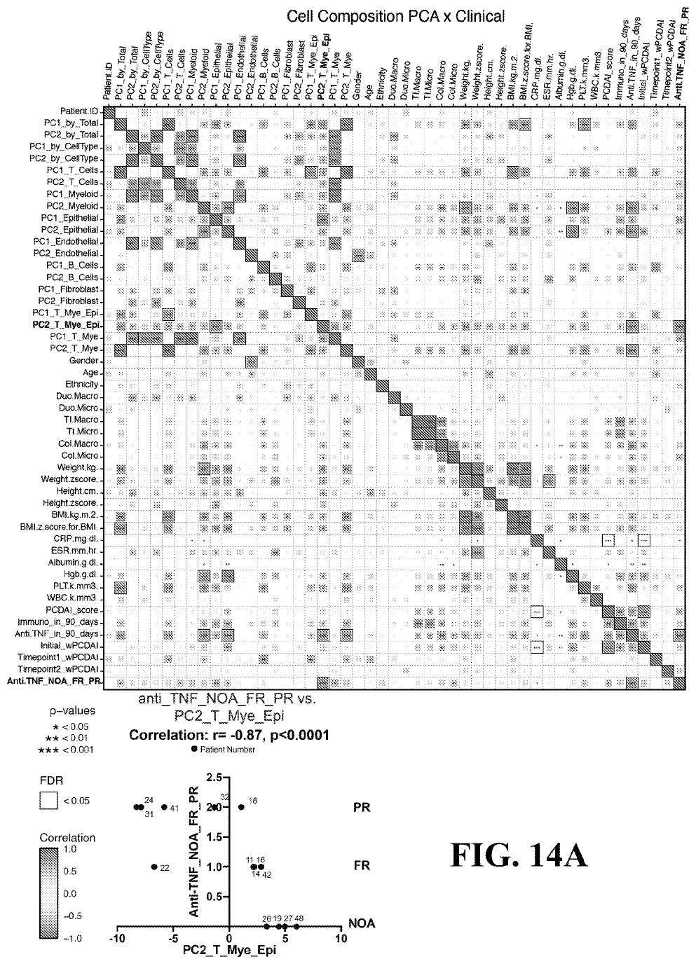

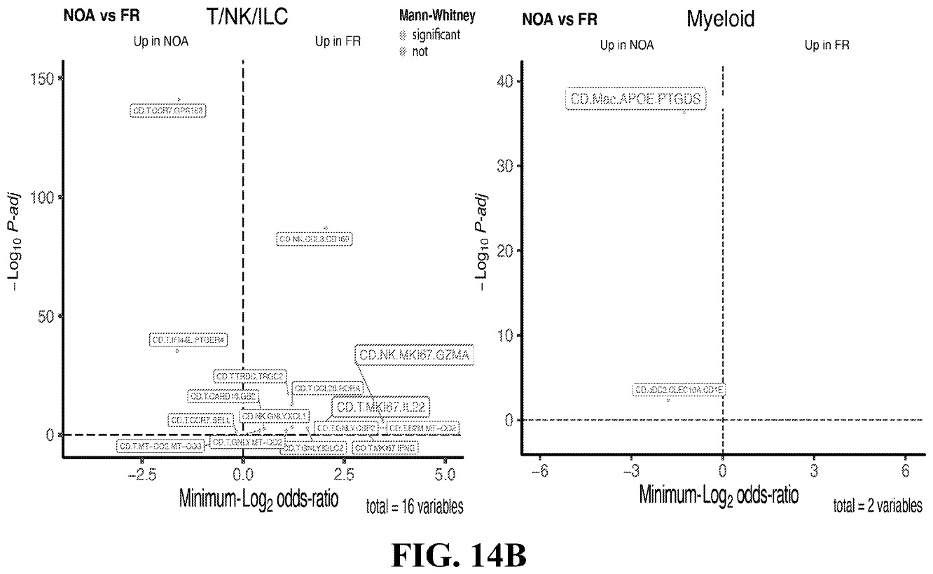

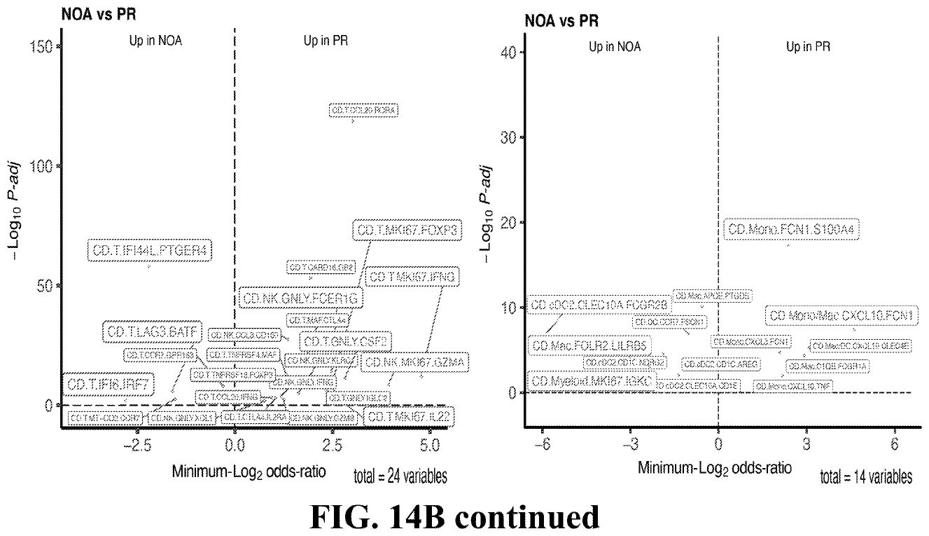

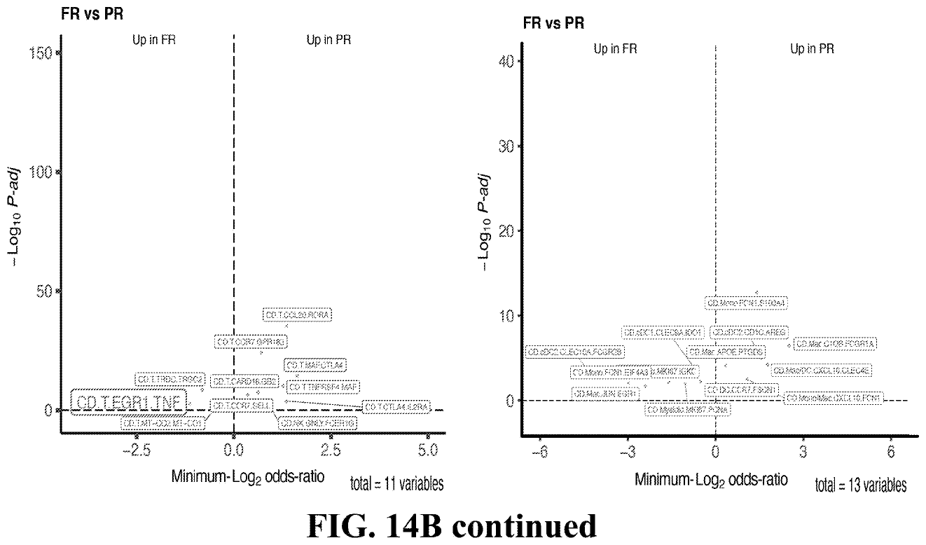

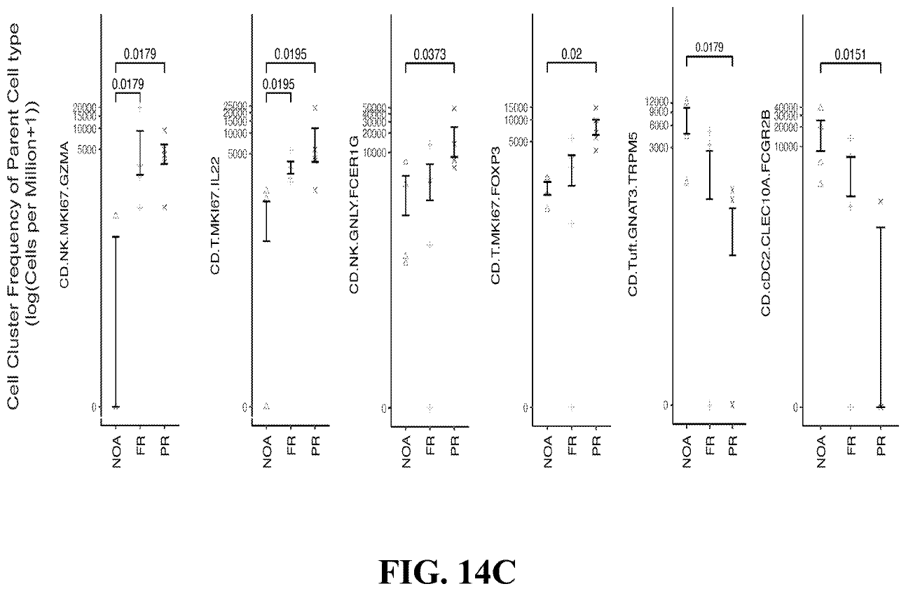

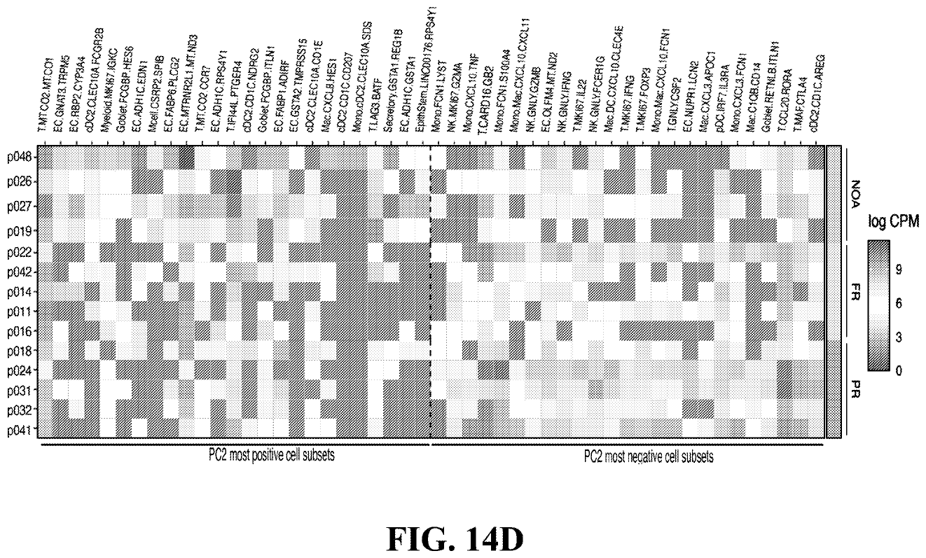

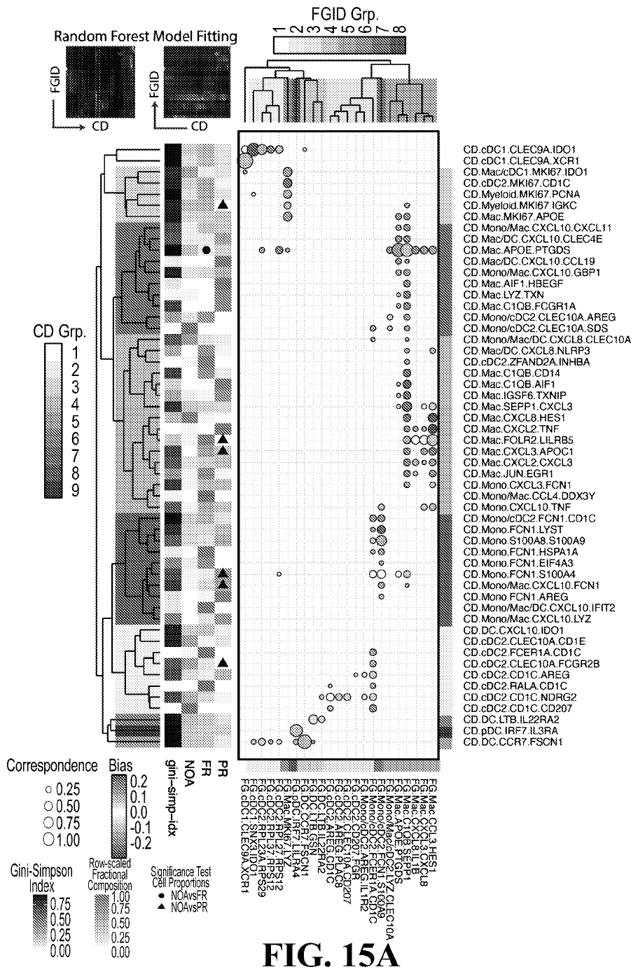

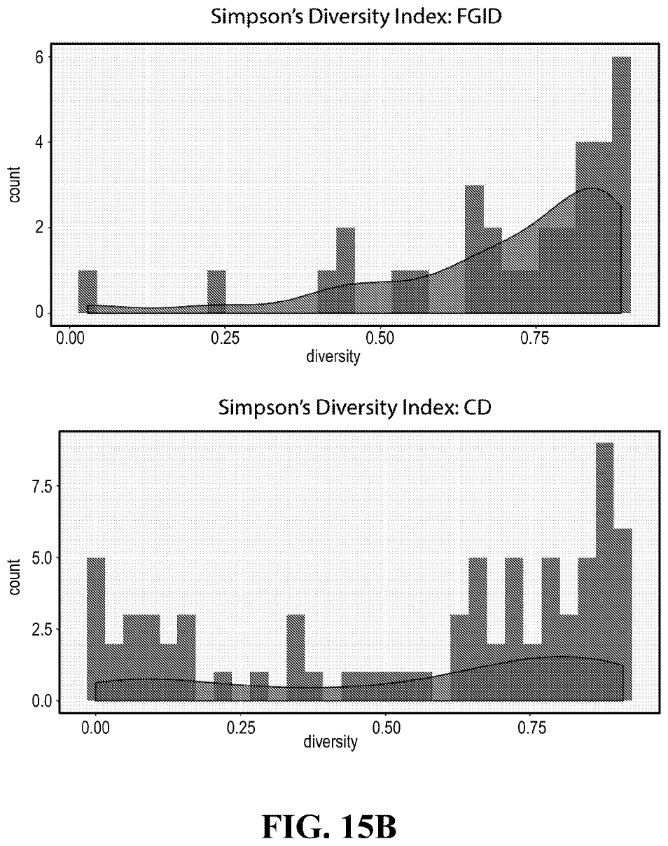

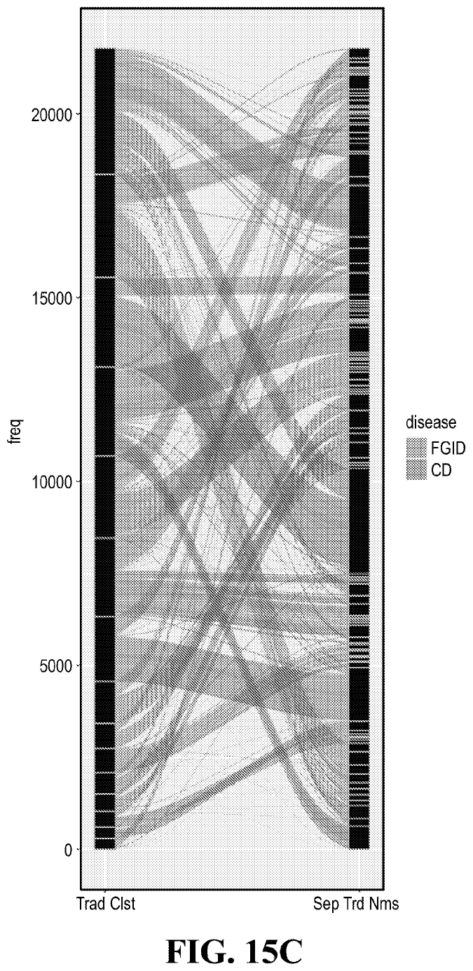

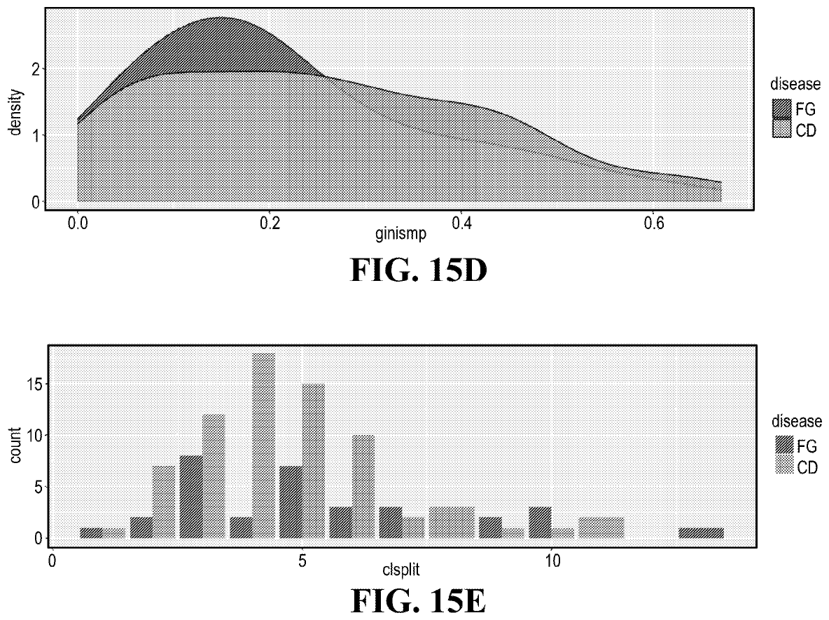

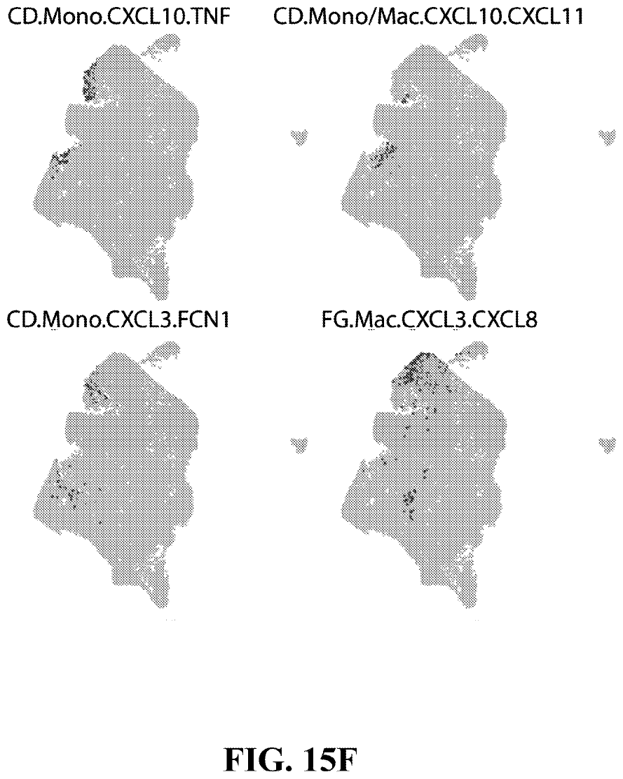

Using this analytical workflow, Applicants present comprehensive cellular atlases of FGID (FIG. 12) and pediCD (FIG. 13), nominate cell states associated with disease severity and treatment outcomes in pediCD (FIG. 14), and identify correspondence between the two (FIG. 15). Applicants focus on pediCD, and those cell states which distinguish between disease severity (NOA vs. PRs/FRs) and baseline gene expression differences in anti-TNF treatment response (FRs vs. PRs).

### Example 12—Comprehensive Atlas of Non-Inflammatory FGID

From 13 FGID patients, Applicants recovered 12 Tier 1 clusters which Applicants display on a t-stochastic neighbor embedding (t-SNE) plot colored by cluster identity containing 99,488 cells (FIG. 12a; FIG. 22b). These Tier 1 clusters represent the main cell types found in the lamina propria and remnant epithelium of an ileal biopsy. Inspecting each individual patient's contribution to the t-SNE, Applicants noted that all patients contributed to all Tier 1 clusters, though note that p044 was overrepresented with more terminally differentiated epithelial cells, likely from incomplete EDTA separation, and thus omit the p044 unique cell clusters from further analyses of composition (FIG. 12b; FIG. 21d; Table 10). Applicants then proceeded to generate preliminary descriptive names based on inspection of each cluster within each tier, calculated a hierarchically-clustered dendrogram, and produced systematic names for each end cell state within each cell Tier 1 cell type (FIGS. 12c,d; FIG. 22; Table 8; Methods). Applicants present top marker genes for each main Tier 1 cluster/cell type, and note that Applicants also provide complete gene lists calculated through Wilcoxon with Bonferroni adjusted p<0.05 for Tier 1 clusters/cell types, subsets, and end cell states (FIG. 12e, Table 4). As patient identity did not factor into ITC stop conditions, Applicants then calculated Simpson's Index of Diversity for each of the clusters (FIG. 12d; FIG. 22; Simpson's Index >0.1). Low diversity clusters may still reflect important biology for individual patients, but Applicants comment more extensively on clusters with high patient diversity.

Within B cells, Applicants identified a strong division between non-cycling and cycling B cells, with those found in the cycling compartment readily identifiable by germinal center markers and further dark zone (AICDA) and light zone (CD83) genes resulting in FG.B/DZ.AICDA.IGKC and FG.B/LZ.CD74.CD83 clusters (FIG. 12d) (Victora et al., 2010).

Within myeloid cells, Applicants identified, and confirmed using extensive inspection of literature curated markers, cell subsets corresponding to monocytes (CD14, FCGR3A, FCN1, S100A8, S100A9, etc.), macrophages (CSF1R, MERTK, MAF, C1QA, etc.), cDC1 (CLEC9A, XCR1, BATF3), cDC2 (FCER1A, CLEC10A, CD1C, IRF4 etc.), and pDCs (IL3RA, LILRA4, IRF7) (FIG. 12d) (Bleriot et al., 2020; Dutertre et al., 2019; Guilliams et al., 2018). Applicants highlight selected cell states including a migratory dendritic cell state (FG.DC.CCR7.FSCN1), extensive cDC2 heterogeneity relative to cDC1 heterogeneity, and a main distinction between macrophage clusters expressing CIQ*, AIP*, APOE, CD68 and PTGDS, (FG.Mac.C1QB.SEPP1, FG.Mac.APOE.PTGDS) and a series of clusters expressing various chemokines including CCL3, CXCL3,and CXCL8 (FG.Mac.CCL3.HES1, FG.Mac.CXCL3.CXCL8, FG.Mac.CXCL8.IL1B).

Within T cells, Applicants followed a similar approach as utilized for Myeloid cells and identified principal cell subsets of T cells (joint expression of CD247, CD3D, CD3E, CD3G with TRAC, TRBC1, TRBC2, or TRGC1, TRGC2 and TRDC), and a combined cluster of cytotoxic cells (FG.T/NK/ILC.GNLY.TYROBP) likely including T cells, NK cells (lower expression of TCR-complex genes with NCAM1, NCR1 and TYROBP), and some TLCs (KIT, NCR2, RORC and low expression of CD3-complex genes) (FIG. 12d) (Cherrier et al., 2018; Robinette and Colonna, 2016). Applicants note that the numerical majority of CD4 T cells (FG.T/NK/TLC.MAF.RPS26) and CD8 T cells (FG.T/NK/ILC.CCR7.SELL) expressed SELL and CCR7 thus identifying them as naïve T cells. However, regardless of clusters expressing CD4 (FG.T.GZMK.GZMA) or CD8A CD8B (FG.T.GZMK.IFNG, FG.T.GZMK.CRTAM, etc.), most activated T cells were characterized by expression of granzymes (Sallusto et al., 1999).

Within epithelial cells, most cells expressed high levels of OLFM4, identifying them as crypt-localized cells (Moor et al., 2018). Applicants readily identified subsets of stem cells (LGR5), proliferating cells (TOP2A), goblet cells (SPINK4, ZG16, various MUCs), enteroendocrine cells (SCG3, ISL1), Paneth cells (ITLN2, PRSS2, LYZ), tuft cells (GNG13, SH2D6, TRPM5) and enterocytes (APOC3, APOA1, FABP6, etc.) (FIG. 12d) (Barker et al., 2007; van der Flier and Clevers, 2009).

Within endothelial cells, Applicants readily identified vascular and lymphatic endothelial cells (LYVE1, PROX1), with the vascular cells able to be further identified as capillaries (CA4) or venular endothelial cells (ACKR1, MADCAM1) (Brulois et al., 2020). Applicants also identified a subset of cells (FG.Endth/Peri.FRZB.NOTCH3) expressing high levels of FRZB and NOTCH3, which, rather than being arterioles, likely represent arteriole-associated pericytes or smooth muscle cells given the absence of EFNB2, SOX17, BMX, and HEY1, and the presence of ACTA2 and MYL9, as cluster-defining genes (FIG. 12d) (Travaglini et al., 2020; Whitsett et al., 2019). Applicants highlight that the FG.Endth/Ven.ACKR1.MADCAM1 cluster is characterized by expression of markers for postcapillary venules specialized in leukocyte recruitment (Thiriot et al., 2017).

Within fibroblasts, Applicants identified principal subsets characterized by their structural roles (COL3A1, ADAMDEC1, FBLN1, LUM, etc.), myofibroblasts (MYH11, ACTA2, ACTG2, etc.), and organization of lymphoid cells (CCL19, CCL21 etc.) (FIG. 12d) (Buechler et al., 2021; Davidson et al., 2021). Within the lymphoid-organizing fibroblasts, Applicants draw attention to the FG.Fibro.C3.FDCSP, FG.Fibro.CCL19.C3, and FG.Fibro,CCL21.CCL19 subsets, which appear to have some characteristics of follicular dendritic cells and variable expression of CCL19 CCL21 (T-cell or migratory dendritic cell chemoattractants) and CXCL13 (B-cell chemoattractant) (Das et al., 2017; Heesters et al., 2013). Applicants also identified a separate Tier 1 cluster of glial cells characterized by CRYAB and CLU. Intriguingly within the glial cell Tier 1 cluster, Applicants then recovered a cell subset expressing FDCSP, CXCL13, and CR2, a key complement receptor which allows for complement-bound antigens to be recycled and presented by follicular dendritic cells (Das et al., 2017; Heesters et al., 2013). This highlights the power of iterative tiered clustering to recover discrete cell states that may, through the process of traditional clustering, not be fully resolved. Furthermore, the presence of these discrete cell clusters within larger parent cell clusters will alter the gene expression signatures of the higher-level cell types. For example, the FG.Glial/fDC.FDCSP.CXCL13 in the hierarchical cluster tree then assorts within the lymphoid-organizing stromal cells.

The mast cells recovered did not further sub-cluster in an automated fashion, and were largely marked by TPSB2 and TPSAB1 (>97%), with minimal CMA1 (<20%) expressing cells, suggesting they are largely classical MC-T cells in FGID intestine (Dwyer et al., 2021).

Applicants identified four Tier 1 clusters for plasma cells, which are characterized by their strong expression of IGH* immunoglobulin heavy-chain genes together with either an IGK* (kappa light chain) or IGL* (lambda light chain) genes (Cyster and Allen, 2019; James et al., 2020). This resolved IgA IgK plasma cells, IgA IgL plasma cells, IgM plasma cells, and IgG plasma cells. Iterative tiered clustering identified further heterogeneity within all clusters of IgA and IgG plasma cells, though given the 3′-bias of this dataset, Applicants note that a principled investigation of these clusters would ideally use 5′ sequencing with targeted VDJ amplification.

Together, the treatment-naïve cell atlas from 13 FGID patients captures 138 cell clusters from a non-inflammatory state of pediatric ileum which Applicants annotated and named in a principled fashion.

### Example 13—Comprehensive Atlas of pediCD

From the 124,054 cells profiled from 14 pediCD patients, Applicants recovered 12 Tier 1 clusters which here Applicants display on a t-SNE plot colored by cluster identity (FIG. 13a). Distinct from FGID, Paneth cells clustered separately at Tier 1, while glial cells were now found within the fibroblast Tier 1 cluster. Inspecting each individual patient's contribution to the t-SNE, Applicants noted that all patients contributed to all Tier 1 clusters (FIG. 13b; FIG. 21c, Table 10). Applicants then proceeded to generate preliminary descriptive names, independently from the FGID atlas, based on inspection of each cluster within each tier, calculate a hierarchically-clustered dendrogram, and provide systematic names for each end cell state within each cell type and subset (FIGS. 13c,d; FIG. 23; Table 9; Methods). Applicants present top marker genes for each main Tier 1 cluster/cell type, and note the complete gene lists calculated through Wilcoxon with Bonferroni adjusted p<0.05 available for Tier 1 clusters/cell types, subsets, and end cell states (Tables 1). Applicants then calculated Simpson's Index of Diversity to denote the patient diversity present within each end cluster, identifying that most clusters in pediCD are conserved across multiple patients, and 16/305 clusters are single-patient clusters (FIG. 13d; FIG. 22; Simpson's Index >0.1). Applicants note that in pediCD, relative to FGID, a higher fraction of cell clusters exhibited lower patient diversity.

Within B cells, Applicants also identified a strong division between non-cycling and cycling B cells, with those found in the cycling compartment readily identifiable by germinal center markers and further dark zone (AICDA) and light zone (CD83) genes, as in FGID (Victora et al., 2010). Within cells expressing germinal centers markers, a highly-proliferative branch including clusters such as CD.B/LZ.CCL22.NPW, CD.B/GC.MKI67.RRM2, and CD.B/DZ.HIST1H1B.MKI67 emerged (FIG. 13d). The CD.B/LZ.CCL22.NPW was characterized by high levels of MYC, which has been shown to allow for further rounds of germinal center affinity maturation (Dominguez-Sola et al., 2012). More numerous B cell clusters included ones characterized by expression of GPR183, such as CD.B.CD69.GPR183 (also expressing IGHG1) and CD.B.RPS29.RPS21. GPR183 has been shown to regulate the positioning of B cells in lymphoid tissues (Pereira et al., 2009).

Within myeloid cells, Applicants identified, and confirmed using the same extensive inspection of literature curated markers as in FGID, cell subsets corresponding to monocytes (CD14, FCGR3A, FCN1, S100A8, S100A9, etc.), macrophages (CSF1R, MERTK, MAF, C1QA, etc.), cDC1 (CLEC9A, XCR1, BATF3), cDC2 (FCER1A, CLEC10A, CD1C, IRF4 etc.), and pDCs (IL3RA, LILRA4, IRF7) (FIG. 13d; FIG. 23) (Bleriot et al., 2020; Dutertre et al., 2019; Guilliams et al., 2018). Applicants highlight selected cell states including a migratory dendritic cell state (CD.DC.CCR7.FSCN1), extensive cDC2 heterogeneity relative to cDC1 heterogeneity, and a main distinction between macrophages expressing CIQ*, MMP*, APOE, CD68 and PTGDS, (CD.Mac.APOE.PTGDS) and a series of clusters expressing various chemokines including CXCL2, CXCL3, and CXCL8 (CD.Mac.SEPP1.CXCL3, CD.Mono.CXCL3.FCN1, CD.Mono.CXCL10.TNF). Several of the end cell clusters initially clustering with macrophages also expressed monocyte markers (S100A8, S100A9), and expressed detectable, but lower levels of MERTK or AXL relative to bonafide macrophages, potentially indicative of the early stages of the trajectory of monocyte-to-macrophage differentiation (Bleriot et al., 2020; Dutertre et al., 2019; Guilliams et al., 2018). Applicants also noted a substantial expansion of clusters characterized by expression of CXCL9, CXCL10, and STAT1, canonical interferon-stimulated genes, observed in clusters such as CD.Mono/Mac.CXCL10.FCN1 (Ziegler et al., 2020, 2021). Moreover, Applicants identified a cluster of inflammatory monocytes, CD.Mono.S100A8.S100A9, characterized by both CD14 and FCGR3A expression.

Within T cells, Applicants followed a similar approach as utilized for FGID T cells and identified cell subsets of T cells (joint expression of CD247, CD3D, CD3E, CD3G with TRAC, TRBC1, TRBC2, or TRGC1, TRGC2 and TRDC), but in pediCD also identified several discrete clusters of NK cells (lower expression of TCR-complex genes with FCGR3A or NCAM1, NCR1 and TYROBP), and ILCs (KIT, NCR2, RORC and low expression of CD3-complex genes) (FIG. 13d, FIG. 23) (Cherrier et al., 2018; Robinette and Colonna, 2016). Applicants note that T cells and NK cells with a shared expression of GNLY, GZMB and other cytotoxic effector genes cluster almost indistinguishably from each other through iterative tiered clustering and visualization of the hierarchical tree, but that careful inspection of literature-curated markers helped resolve NK cells (CD.NK.CCL3.CD160; CD.NK.GNLY.GZMB) from CD8A/CD8B T cells (CD.T.GNLY.GZMH; CD.T.GNLY.CTSW) (FIG. 13d, FIG. 23) (Cherrier et al., 2018; Robinette and Colonna, 2016). One of the specific challenges in distinguishing between T cells and NK cells in scRNA-seq data is that NK cells can express several CD3-complex genes, particularly CD247, as well as detectable aligned reads for TRDC or TRBC1 and TRBC2, and thus lower-resolution clustering approaches or datasets with lower cell numbers may miss these important distinctions (Bjorklund et al., 2016; Renoux et al., 2015). NK cell clusters also expressed the highest levels of TYROBP, which encodes DAP12 and mediates signaling downstream from many NK receptors (French et al., 2006; Lanier, 2001; Lanier et al., 1998). ILC clusters such as CD.ILC.LST1.AREG or CD.ILC.IL22.KIT were characterized by an apparent ILC3 phenotype, with expression of KIT, RORC and IL22, though they also expressed detectable transcripts of GATA3 in the same clusters (Cherrier et al., 2018; Robinette and Colonna, 2016). Applicants detected several clusters expressing CD4 and lacking CD8A CD8B, including regulatory T cells (CD.T.TNFRSF18.FOXP3), and MAF- and CCR6-expressing helper T cells (CD.T.MAF.CTLA4). Perhaps most strikingly, Applicants resolved multiple subsets of proliferating lymphocytes, including regulatory T cells (CD.T.MKI67.FOXP3), IFNG-expressing T cells (CD.T.MKI67.IFNG), and NK cells (CD.NK.MKI67.GZMA).

Within epithelial cells Applicants identified substantial heterogeneity in CD. Most cells expressed high levels of OLFM4, identifying them as crypt-localized cells (Moor et al., 2018). Applicants readily identified subsets of stem cells (LGR5), proliferating cells (TOP2A), goblet cells (SPINK4, ZG16, various MUCs), enteroendocrine cells (SCG3, ISL1), Paneth cells (ITLN2, PRSS2, LYZ), tuft cells (GNG13, SH2D6, TRPM5) and enterocytes (APOC3, APOA1, FABP6, etc.) (FIG. 13d) (Barker et al., 2007; van der Flier and Clevers, 2009). Amongst several clusters characterized by CCL25 and OLFM4 expression, Applicants identified a subset marked by LGR5 expression, characteristic of intestinal stem cells (CD.EpithStem.LINC00176.RPS4YA1) (Barker et al., 2007). Applicants identified several subsets expressing CD24, indicative of crypt localization, with expression of REG1B (CD.Secretory.GSTA1.REG1B; CD.Secretory.REG1B.REG1A) (Moor et al., 2018). Applicants also identified early enterocyte cluster CD.EC.ANPEP.DUOX2, characterized by FABP4 and ALDOB and expressing DUOX2 and MUC1. Applicants resolved several clusters of enteroendocrine cells, including CD.Enteroendocrine.TFPI2.TPH1 and CD.Enteroendocrine.NEUROG3.MLN. Applicants also found two clusters Applicants labeled as M cells based on expression of SPIB (CD.Mcell.CCL23.SPIB; CD.MCell.CSRP2.SPIB) (Beumer et al., 2020; Mabbott et al., 2013). Paneth cells did not further sub-cluster despite forming an independent Tier 1 cluster (CD.Epith.Paneth). Most strikingly, Applicants identified a diversity of goblet cells recovered across multiple patients including CD.Goblet.HES6.COLCA2 expressing REG4 and LGALS9, and CD.Goblet.TFF1.TPSG1 expressing TFF1 and ITLN1, amongst others. Applicants also identified a cluster of Tuft cells: CD.EC.GNAT3.TRPM5.

Within endothelial cells, Applicants also readily identified vascular and lymphatic endothelial cells (LYVE1, PROX1), with the vascular cells able to be further identified as capillaries (CA4) or venular endothelial cells (ACKR1, MADCAM1) (FIG. 13d). Applicants also identified a subset of cells (CD.Endth/Mural.HIGD1B.NDUFA4L2) expressing high levels of FRZB and NOTCH3, which, rather than being arterioles, likely represent arteriole-associated pericytes or smooth muscle cells given the absence of EFNB2, SOX17, BMX, and HEY1, and the presence of ACTA2 and MYL9, as cluster-defining genes. In pediCD, Applicants also identified a cluster of arteriolar endothelial cells, CD.Endth/Art.SEMA3G.SSUH2, identified by expression of HEY1, EFNB2, and SOX17. Applicants also highlight that the endothelial venules characterized by expression of markers for postcapillary venules specialized in leukocyte recruitment, such as CD.Endth/Ven.ADGRG6.ACKR1 and CD.Endth/Ven.POSTN.ACKR1, exhibited greater diversity than in FGID with multiple end cell clusters identified (Thiriot et al., 2017).

Within fibroblasts, Applicants identified principal subsets characterized by their structural roles (COL3A1, ADAMDEC1, FBLN1, LUM, etc.), myofibroblasts (MYH11, ACTA2, ACTG2, etc.), and organization of lymphoid cells (CCL19, CCL21 etc.) (FIG. 13d) (Buechler et al., 2021; Davidson et al., 2021). The principal hierarchy in fibroblasts in pediCD was between FRZB-, EDRNB- and F3-expressing subsets such as CD.Fibro.LY6H.PAPPA2 and CD.Fibro.AGT.F3, which were also enriched for CTGF and MMP1 expression, and ADAMDEC1-expressing fibroblasts, which were enriched for several chemokines such as CXCL12, and in some specific clusters CXCL6, CXCL1, CCL11, and other chemokines. Amongst three fibroblast subsets marked by C3 expression, Applicants identified follicular dendritic cells (CD.Fibro/fDC.FCSP.CXCL13), along with fibroblasts expressing CCL21, CCL19, and the interferon-stimulated chemokines CXCL9 and CXCL10 (CD.Fibro.CCL21.CCL19; CD.Fibro.TNFSF11.CD24) (Das et al., 2017; Heesters et al., 2013). Distinct from the FGID atlas, within the pediCD atlas, glial cells clustered within fibroblasts, but were also marked by S100B, PLP1 and SPP1 expression.

The mast cells recovered in pediCD did further sub-cluster in an automated fashion, were largely marked by TPSB2 (>90%), with minimal CMA1 (<16%) expressing cells, suggesting they are largely classical MC-T cells in pediCD intestine (FIG. 13d) (Dwyer et al., 2021). Intriguingly, some subsets (CD.Mstcl.AREG.ADCYAP1) were enriched for IL13-expression. Applicants also detected a small cluster of proliferating mast cells from several patients (CD.Mstcl.CDK1.KIAA0101).

Applicants also identified four Tier 1 clusters for plasma cells, which are characterized by their strong expression of IGH* immunoglobulin heavy-chain genes together with either a IGK* (kappa light chain) or IGL* (lambda light chain) genes. This resolved IgA IgK plasma cells, IgA IgL plasma cells, IgM plasma cells, and IgG plasma cells. Iterative tiered clustering identified further heterogeneity within all clusters of IgA plasma cells, though given the 3′-bias of this dataset, Applicants note that a principled investigation of these clusters would ideally use 5′ sequencing with targeted VDJ amplification.

Together, the treatment-naïve cell atlas from 14 pediCD patients captures 305 cell clusters from an inflammatory state of the pediatric ileum suggesting an increase in the number and diversity of cell states present in the intestine during overt inflammatory disease.

### Example 14—Clinical Variables and Cellular Variance that Associates with pediCD Severity

As this pediCD atlas was curated from treatment-naïve diagnostic samples, Applicants were able to interrogate the data to test if overall shifts in cellular composition, specific cell states, and/or gene expression signatures underlie clinically-appreciated disease severity and treatment decisions (NOA vs. FR/PR), and those that are further associated with response to anti-TNF therapies (either FRs or PRs). Here, Applicants leveraged the detailed clinical trajectories collected from all patients as the ultimate functional test: resolving how cellular composition and cell states predict disease and treatment outcomes.

In order to capture the overall principal axes of variation explaining changes in cellular composition, Applicants calculated the fractional composition of all 305 end cell clusters in pediCD within its parent cell type (“per cell type”), or within all cells (“per total cells”), and performed a principal component analysis (PCA) over both of these sample x cell cluster frequency tables (Table 11) (Mathew et al., 2020). Applicants then used the PC1 (13.4% variation “per cell type” and 13.5% variation “per total cells”) and PC2 (12.7% variation “per cell type” and 11.8% variation “per total cells”) as numerical variables which Applicants correlated with clinical metadata including categorical variables (patient ID, ethnicity, gender, etc.), ordinal variables (Terminal Ileum (TI)-macroscopic endoscopic evidence, TI-microscopic histopathology, Anti-TNF treatment within 90 days of diagnosis, and treatment decision/response coded as anti-TNF_NOA_FR_PR, etc.) and numerical variables (Height, BMI, CRP, ESR, PLT, PCDAI, wPCDAI, etc.) (FIG. 14a, r by Spearman-rank). Amongst the clinical variables, Applicants noted strong correlation between Initial wPCDAI and CRP (r-0.83), and moderate correlation between Initial wPCDAI and anti-TNF within 30 days (r=0.65) and anti-TNF_NOA_FR_PR (r=0.49). For PC1-“per total cells”, Applicants identified strong correlations with anti-TNF treatment within 90 days (r=−0.76), and moderate correlation anti-TNF_NOA_FR_PR (r=−0.58; Methods). For PC1-“per cell type”, Applicants identified strong correlation with anti-TNF within 90 days (r=−0.72), and moderate correlation with anti-TNF_NOA_FR_PR status (r=−0.63). PC1-“per cell type” was also strongly correlated with BMI and PC1-“per total cells” (r>−0.7). PC1-“per cell type” was weakly correlated with patient ID and gender (r<0.3).

In order to understand if any cell types were predominantly driving associations with clinical disease severity at initial presentation, Applicants then further deconvoluted the overall PCA on 305 end clusters and performed PCA over each cell type's fractional composition of end clusters individually (B cells: 33 clusters, Endothelial: 18 clusters, Epithelial: 68 clusters, Fibroblast 45 clusters, Myeloid: 54 clusters, T/NK/ILCs: 57 clusters), and correlated the first two PC's (all PC1's and PC2's each accounted for >13% variance) with all of the clinical variables (Table 11). The PCs derived from T/NK/ILC cells, myeloid cells, and epithelial cells were all moderately correlated with anti-TNF_NOA_FR_PR status (r>0.49) individually and had higher values than the other cell types; therefore, Applicants asked if a PCA-based metric considering all three cell types would synergistically capture both disease severity and treatment response. When Applicants calculated the PCA accounting for frequencies within each cell type of T/NK/ILC cells, myeloid cells, and epithelial cells, Applicants found strong correlation for PC2 with both anti-TNF within 90 days (r=−0.83) and anti-TNF-NOA_FR_PR status (r=−0.87) (FIG. 14a). This represented the two strongest correlations of any variable Applicants tested with anti-TNF treatment and response status, outperforming wPCDAI.

### Example 15—Discrete Cell Cluster Changes Across the pediCD Clinical Severity and Response Spectrum

Applicants next focused on further deconstructing the disease severity vector: identifying which cell clusters accounted for the most significant changes in abundance based on the relative frequency of an end cell cluster within its parent cell type. Applicants focus on this form of analysis for scRNA-seq, similar to what is typically reported for flow cytometry, and further discuss approaches to enumerate total cell numbers which would be critical to identify changes in overall cellularity in the different pediCD treatment and response categories (Discussion) (Gomariz et al., 2018). Applicants first performed a Fisher's exact test between NOA vs. FR; NOA vs. PR; or FR vs. PR, and then performed a Mann-Whitney U test to highlight specific clusters and discuss results from clusters with high Simpson's index of diversity (i.e. recovered from multiple patients) as shown for T/NK/ILCs and Myeloid Cell Types (FIG. 14b; Methods).

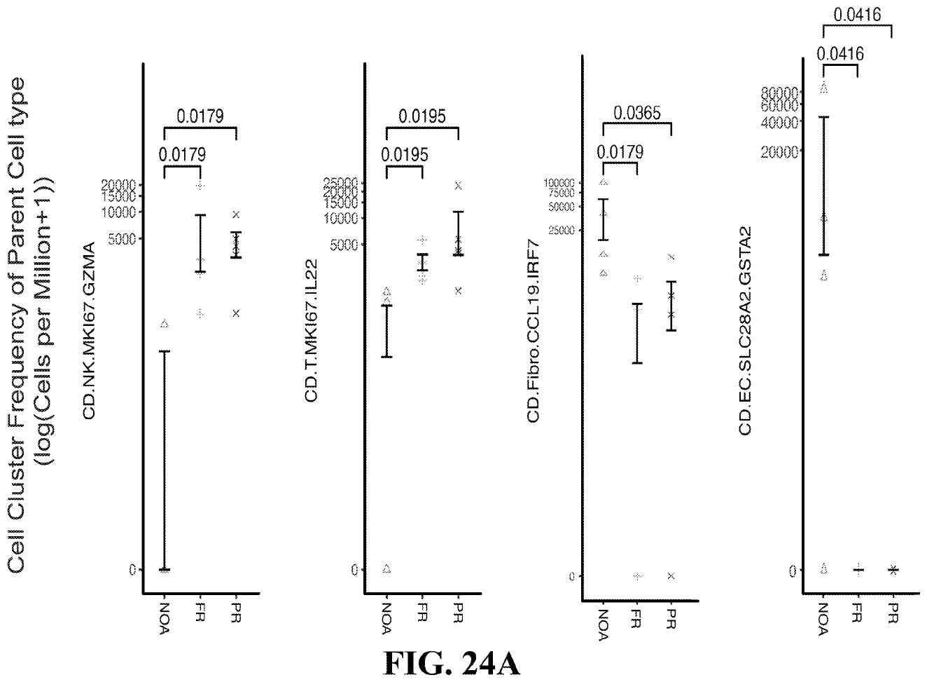

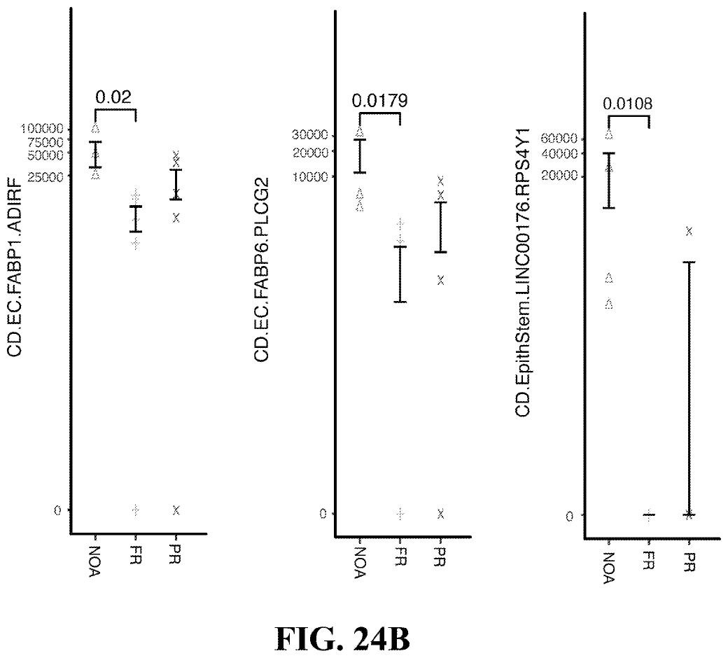

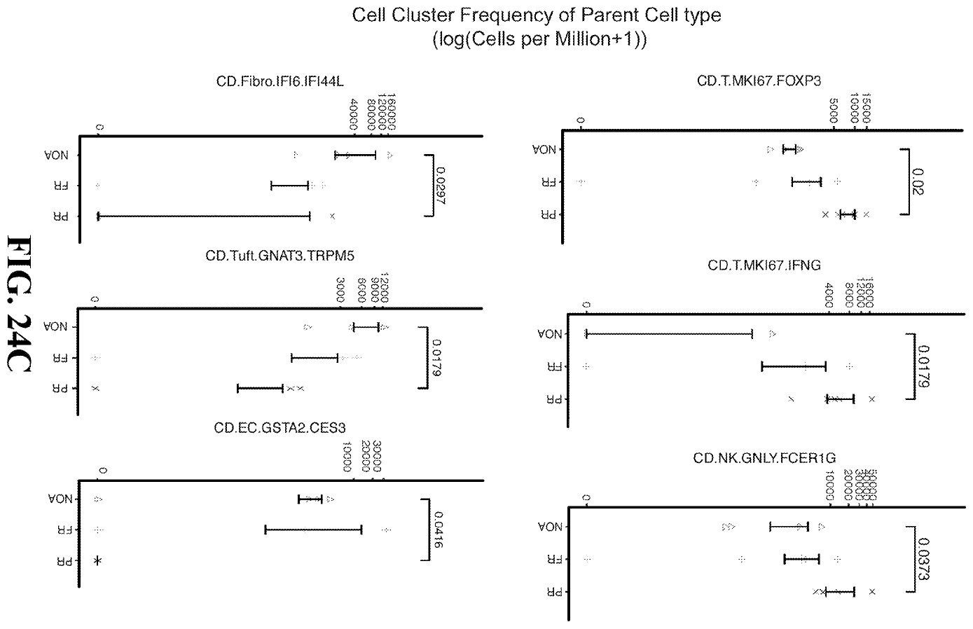

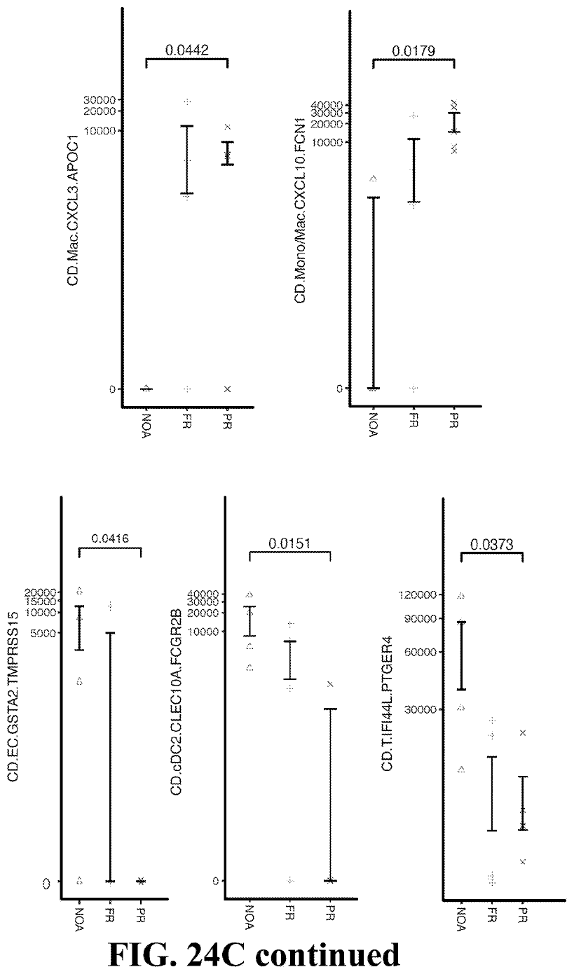

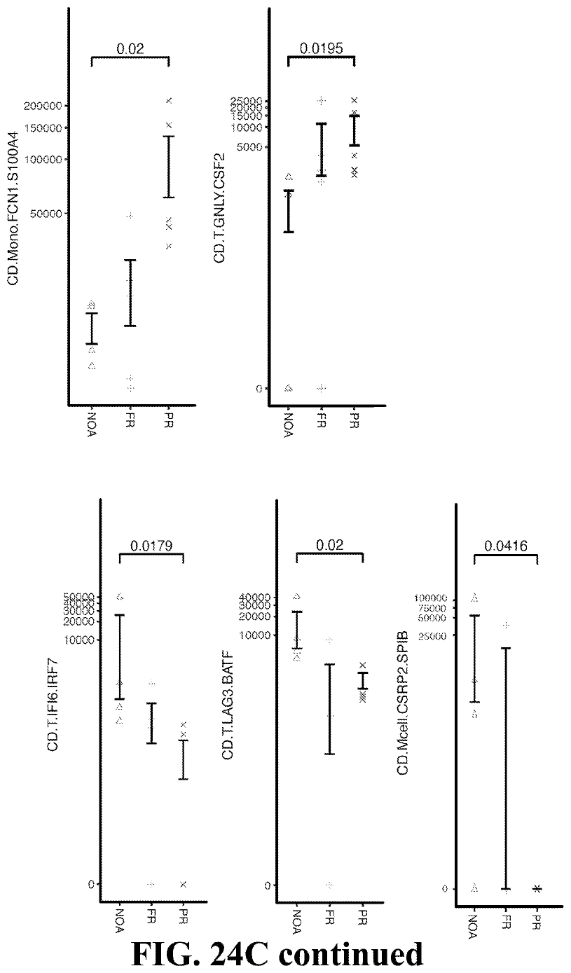

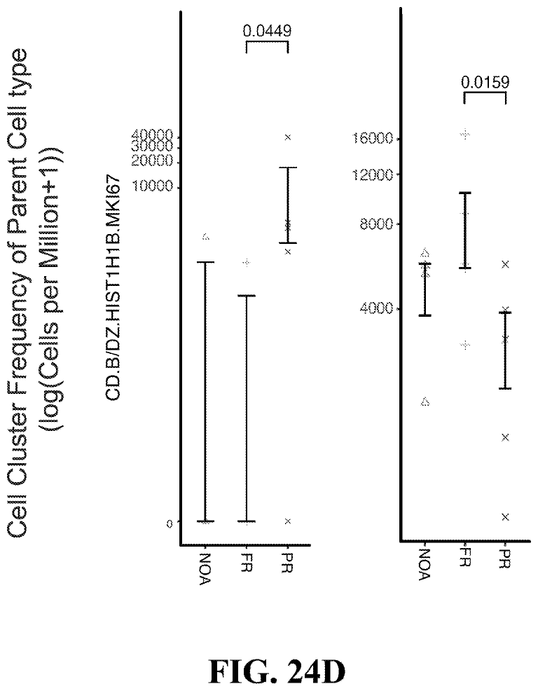

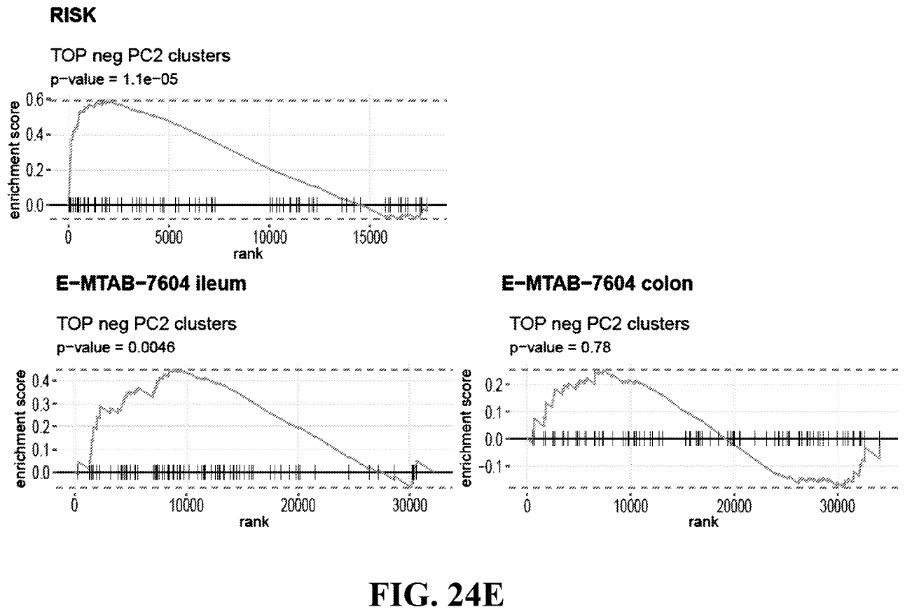

When comparing FR/PRs to NOAs, two subsets with significantly increased frequency in FR/PR patients amongst T cells, NK cells, and ILCs were identified. These were CD.NK.MKI67.GZMA and CD.T.MKI67.IL22 (FIG. 14b,c; FIG. 24a; Table 12). Beyond the strong proliferation signature, CD.NK.MKI67.GZMA were enriched for genes such as GNLY, CCL3, KLRD1, IL2RB and EOMES, and CD.T.MKI67.IL22 were enriched for IFNG, CCL20, IL22, IL26, CD40LG and ITGAE. This indicates that with increasing pediCD clinical severity, there is increasing local proliferation of cytotoxic NK cells, and proliferation of tissue-resident T cells with the capacity to express anti-microbial and tissue-reparative cytokines, and molecules to interface with antigen-presenting cells and B cells. Alongside this increase, there was a significant decrease amongst fibroblasts of CD.Fibro.CCL19.IRF7, and amongst epithelial cells of CD.EC.SLC28A2.GSTA2 clusters in the FR/PR patients compared to NOA (FIG. 24a). The CD.Fibro.CCL19.IRF7 were enriched for CCL19, CCL11, CXCL1, CCL2, and very specifically for OAS1 and IRF7. The CD.EC.SLC28A2.GSTA2 cluster was characterized by its two namesake markers, involved in purine transport and glutathione metabolism (Moor et al., 2018).

Applicants also detected significant decreases in FRs relative to NOAs in certain cell types, particularly within Epithelial cells including CD.EpithStem.LINC00176.RPS4Y1, CD.MCell.CSRP2.SPIB, CD.EC.FABP6.PLCG2, and CD.EC.FABP1.ADIRF (FIG. 24b; Table 12). Applicants note that the relative decrease in M cells is in stark contrast to the “ectopic” M-like cells that were detected in adult ulcerative colitis (Smillie et al., 2019).

Applicants next focused on those cell subsets that were significantly changed only between PRs and NOAs (FIG. 14c; FIG. 24c; Table 9). Here Applicants note several more distinct clusters within the lymphocyte cell type, including increases in the PR patients compared to NOA patients of CD.T.MKI67.IFNG, CD.T.MKI67.FOXP3, CD.T.GNLY.CSF2, and CD.NK.GNLY.FCER1G. The two MKI67 clusters again highlighted an increase in proliferative cells, specifically cells enriched for IFNG, GNLY, HOPX, ITGAE and IL26 (CD.T.MKI67.IFNG), and IL2RA, BATF, CTLA4, TNFRSF1B, CXCR3, and FOXP3 (CD.T.MKI67.FOXP3), the latter of which may be indicative of proliferating regulatory T cells. The two GNLY clusters emphasized cytotoxicity, specifically cell clusters were both enriched for GNLY, GZMB, GZMA, PRF1 and more specifically for IFNG, CXCR6, and CSF2 (CD.T.GNLY.CSF2), or AREG, TYROBP, and KLRF1 (CD.NK.GNLY.FCER1G). Amongst myeloid cells, there was an increase in CD.Mac.CXCL3.APOC1, CD.Mono/Mac.CXCL10.FCN1, and CD.Mono.FCN1.S100A4 in PR versus NOA. The CD.Mac.CXCL3.APOC1 cluster was enriched for a variety of chemokines including CCL3, CCL4, CXCL3, CXCL2, CXCL1, CCL20, and CCL8. It was also enriched for TNF and IL1B. The CD.Mono/Mac.CXCL10.FCN1 cluster was enriched for CXCL9, CXCL10, CXCL11, GBP1, GBP2, GBP4, GBP5, suggestive of activation by IFN, and more specifically Type II IFNγ, based on the GBP gene cluster (Ziegler et al., 2020). CD.Mono.FCN1.S100A4 was characterized by S100A4, S100A6, and FCN1 expression. These two immune clusters were paralleled by increases in certain clusters within endothelial cells in PR versus NOA patients (CD.Endth/Ven.LAMP3.LIPG) and epithelial cells (CD.Goblet.TFF1.TPSG1).

Several clusters of cells were decreased in PR versus NOA, including CD.T.LAG3.BATF, CD.T.IFI44L.PTGER4, and CD.T.IFI6.IRF7 amongst lymphocytes (FIG. 24c) (Roncarolo et al., 2018). Amongst myeloid cells, CD.cDC2.CLEC10A.FCGR2B were decreased, and amongst fibroblasts CD.Fibro.IFI6.IFI44L were decreased. In epithelial cells, CD.Tuft.GNAT3.TRPM5 cells were decreased. Alongside the decrease in Tuft cells amongst epithelial cells, two more clusters closely related to the aforementioned CD.EC.GSTA2.SLC28A3 cluster, also marked by GSTA2 expression, were significantly decreased (CD.EC.GSTA2.CES3, and CD.EC.GSTA2.TMPRSS15).

Applicants assessed the compositional differences between FRs and PRs and only identified one cell cluster which was significantly increased in PRs: CD.B/DZ.HIST1H1B.MKI67, which are proliferating dark zone B cells. CD.T.EGR1.TNF T cells were significantly decreased in PR versus FR (FIG. 24d; Table 12). These data suggest that at the earlier stages of pediCD, there are a series of changes in multiple cell types that encapsulate the distinctions between NOA and FR or PR patients.

### Example 16—Collective Cell Vectors Delineating pediCD Clinical Severity and Response Spectrum

Together, the significant changes in cell composition between the clinically-defined patient groups were particularly notable within proliferating T cells, cytotoxic NK cells, monocytes/macrophages, and epithelial cells that could be combined to calculate a numerical variable for “PC2-T/NK/ILC/Myeloid/Epithelial” which correlated strongly with both the clinical presentation leading to a decision to treat or not with anti-TNF therapies and to the distinction between anti-TNF_NOA_FR_PR status (FIG. 14a). Some of the top negative loadings for PC2, enriched in PRs and FRs compared to NOAs, included both helper and cytotoxic T cell clusters (CD.T.MAF.CTLA4; CD.T.CCL20.RORA; CD.T.GNLY.CSF2), NK cell clusters (CD.NK.GNLY.FCER1G; CD.NK.GNLY.IFNG; CD.NK.GNLY.GZMB); proliferating T cells and NK cells (CD.T.MKI67.FOXP3; CD.T.MKI67.IFNG; CD.T.MKI67.IL22; CD.NK.MKI67.GZMA), and monocytes, macrophages, DCs and pDCs (CD.cDC2.CD1C.AREG; CD.Mac.C1QB.CD14; CD.Mono.CXCL3.FCN1; CD.pDC.IRF7.IL3RA; CD.Mono/Mac.CXCL10.FCN1) (FIG. 14d; Table 13). The top positive loadings for PC2 encompassing the NOA-enriched clusters included several epithelial cell subsets such as Tuft cells (CD.EC.GNAT3.TRPM5) and those with specialized metabolic features including retinol-binding, bile binding and export, fatty-acid and cholesterol metabolism, fructose and glucose metabolism, starch metabolism glutathione metabolism, sulfation, and the terminal degradation of peptides (CD.EC.RBP2.CYP3A4; CD.EC.FABP6.PLCG2; CD.EC.FABP1.ADIRF; CD.EC.GSTA2.TMPRSS15) (FIG. 14d; Table 13) (Lampen et al., 2000; Mirtensson et al., 1990; Martinez-Augustin and de Medina, 2008; Sullivan et al., 2021; Wen and Rawls, 2020). Furthermore, clusters also enriched in NOA PC2 such as CD.EC.ADH1C.RPS4Y1 and CD.EC.ADH1C.GSTA1, clustered in a separate branch together and expressed several enzymes responsible for steroid hormone and dopamine biosynthesis (FIG. 4d, 5d) (Cima et al., 2004; Magro et al., 2002). Importantly for the regenerative potential of the epithelium, CD.EpithStem.LINC00176.RPS4Y1 were also defining of the PC2-positive NOA direction. This suggests that multiple collective changes in the composition and/or state of T/NK/ILC cells, myeloid cells, and epithelial cells at diagnosis may help stratify pediCD patients not only by clinically appreciated disease severity but also may influence anti-TNF responsiveness.

To determine whether the disease severity gene signature that Applicants discovered in the PREDICT study can be found in other cohorts, Applicants selected the top 92 markers of the 25 cell states associated with disease severity and treatment outcomes (Table 14) and performed a gene-set enrichment analysis (GSEA) (FIG. 24e). In the two independent treatment-naïve cohorts that Applicants analyzed (the pediatric RISK cohort, n=69, and the adult E-MTAB-7604 cohort, n=43) the gene signature was significantly enriched in illeal, but not colonic, mucosal biopsies from patients who did not respond to anti-TNF therapy compared to those who responded (Kugathasan et al., 2017; Verstockt et al., 2019). Thus, these genes (TNFAIP6, GZMB, S100A8, CSF2, CLEC4E, S100A9, ILIRN, FCGRIA, CLIC3, CD14, PLA2G7, FAM26F, IL3RA, NKG7, IL32, CCL3, OLR1, LILRA4, APOC1, MYBL2) informed by the PC2 cellular vector, and showing best ranks in both cohorts, could potentially serve as predictive markers of anti-TNF therapy outcome in newly diagnosed patients.

### Example 17—Random Forest Classifier Applied to Cellular Taxonomies Allows for Identification of Correspondence Between FGID and pediCD

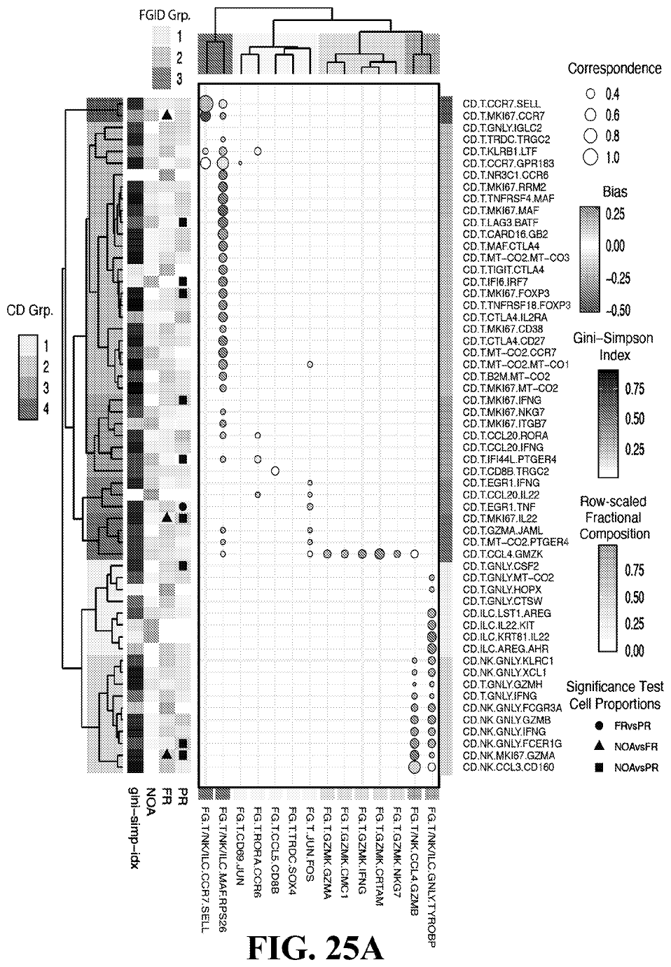

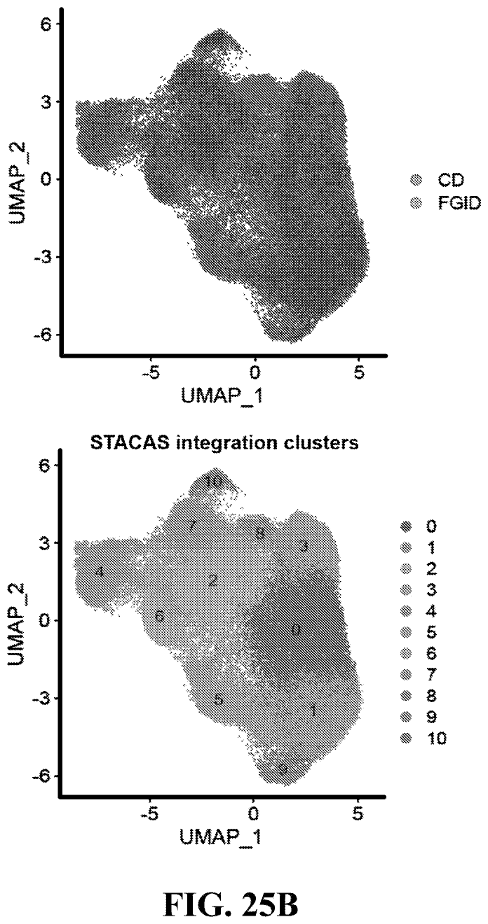

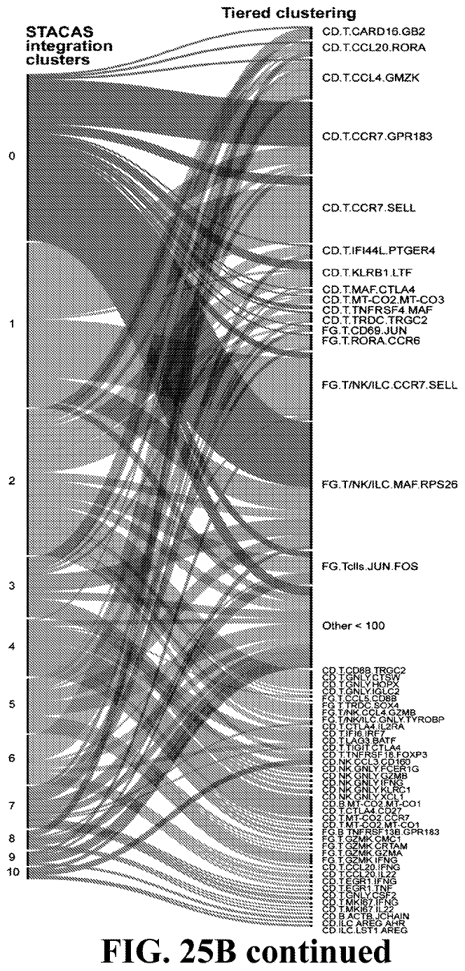

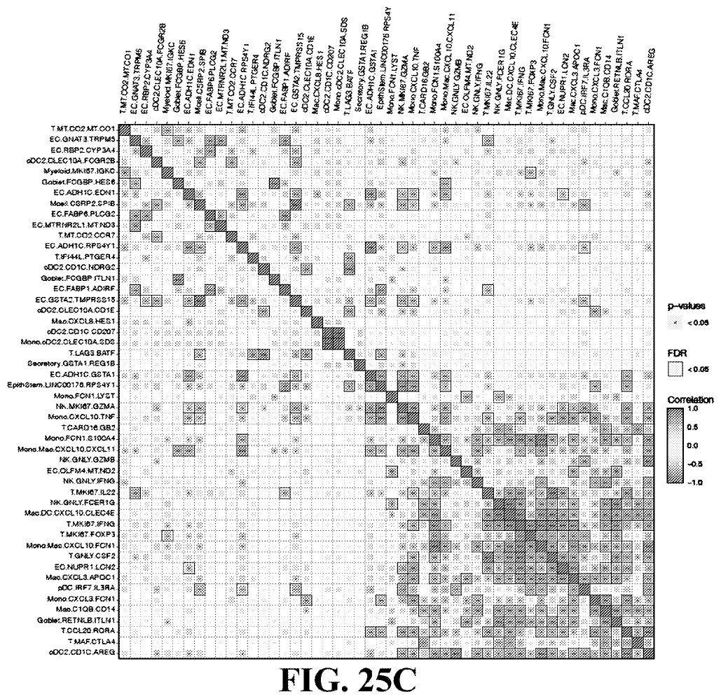

As Applicants had generated independent cellular atlases for FGID and pediCD to mitigate “discovery” of hybrid cell clusters that may not represent bona fide biological cell states, Applicants next sought to match and identify correspondence between pediCD and FGID cell subsets. As the study progressed, several analytical methods to integrate scRNA-seq emerged which utilize distinct principles to either predict cell type names given reference gene lists or directly integrate two datasets that were collected from distinct perturbations, tissues, or even species (Hao et al., 2021; Hie et al., 2019; Korsunsky et al., 2019; Pliner et al., 2019). However, many of these methods are benchmarked on broad cell type or subset integration, and thus their applicability for fine cell states, as in the end clusters Applicants identify here, remains unknown. Thus, Applicants employed a random forest (RF) classifier-based approach, which has recently also been applied successfully in work to identify correspondence in fine sub-clusters in the mammalian retina (Peng et al., 2019; Shekhar et al., 2016). Specifically, Applicants employed paired RF models (one trained on FGID the other trained on pediCD) to obtain cross dataset predictions per cell. Applicants trained these models in scikit-learn with 5-fold cross validation and params: min_samples_leaf=1, oob_score=True, criterion=“gini”, max_depth=200, n_estimators=700, max_features=“sqrt” (Pedregosa et al., 2011). The training set (but not the test set) was sampled with replacement such that all classes contained as many samples as the maximum proportioned class. This up-sampling procedure provided the largest gain to the test accuracy, sensitivity, and specificity scores, increasing accuracy ˜10-15% across each cell type. With the final model, Applicants attained cross-dataset predictions (pediCD to FGID & FGID to pediCD) for each cell, giving a probability score of a cell belonging to a subset in the other disease condition (FIG. 22c; Methods). Applicants applied these models to each cell type individually, and here focus the discussion on Myeloid cells and T/NK/ILC cells as two cell types prominently associated with pediCD disease severity (FIG. 15, FIG. 25). As newer methods are developed, more refined integration is likely to be possible.

Comparing across myeloid cells between pediCD and FGID, Applicants could identify strong correspondence of specific cell subsets such as cDC1s or pDCs (FIG. 15a). Applicants also identified strong correspondence between several cDC2 clusters. Applicants identified a gradient of monocyte and macrophage correspondence of 31 clusters in pediCD to 2 FGID clusters, likely reflective of inflammatory monocyte to macrophage differentiation in pediCD (Bleriot et al., 2020; Dutertre et al., 2019; Guilliams et al., 2018). Some clusters characterized by STAT1 activation did not demonstrate significant correspondence to any FGID cluster. Applicants also generally noted substantially increased cluster diversity in pediCD end clusters relative to their correspondence in FGID. This emerged from more patient-specific clusters found in pediCD, and an overall decrease in Simpson's index of diversity considering the patient composition of each end clusters (FIG. 15b).

For T/NK/ILC cells, Applicants identified more discrete patterns relative to Myeloid cells based on comparison of the RF result. Within the two FGID cytotoxic T cell clusters, Applicants identified correspondence by 18 pediCD clusters, representing ILC3s, and cytotoxic NK cells and T cells (FIG. 25). The cluster of naïve T cells in FGID had correspondence with the majority of pediCD non-cytotoxic T cell clusters, illustrating a substantial activation and specialization to several discrete T cell states that were specific for pediCD.

Importantly, when Applicants jointly clustered macrophages from FGID and pediCD together, Applicants identified that several of the original end clusters identified through ARBOL in pediCD were divided across the UMAP: being split into distinct clusters of cells (FIG. 15c-f). This reinforces the need to quantitatively approach the choice of clustering parameters and number of iterations used. Applicants also employed the STACAS package to integrate T cells between FGID, confirming the higher percentage of proliferating T cells in pediCD patients compared to FGID (FIG. 25b) (Andreatta and Carmona, 2021). However, as compared to ITC, the integration approach on T cells resulted in lower heterogeneity, thus masking important differences revealed by the newly developed ARBOL approach. The detailed analysis of the correspondence between the FGID and pediCD atlases highlights the challenge of multiple cell type, subset, and state vectors which are simultaneously accounted for by clustering over a set of highly variable genes jointly derived from multiple cells and disease conditions. In an atlas composed of multiple clinical entities, highly homogenous cell clusters may appear dispersed across a space based on the other cells that they are being compared against and the parameters used for clustering. This underscores the power in the disease-specific clustering heuristic employed with this dataset, which revealed principled end clusters in pediCD which could then be related back to a non-inflammatory reference atlas while still maintaining fine granularity.

### Example 18—the Phenotypic Space of Macrophages and T Cells is Significantly Different Across FGID and NOA/FR/PR pediCD

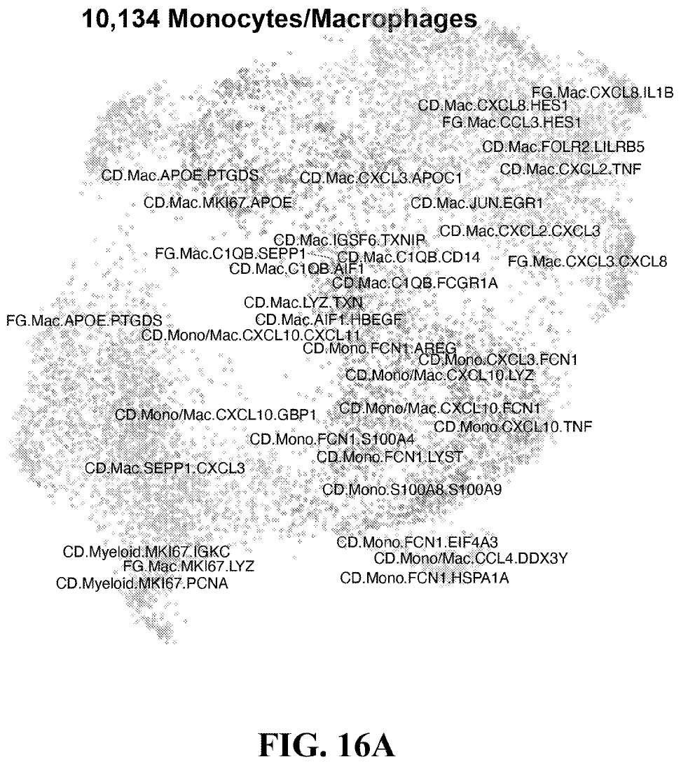

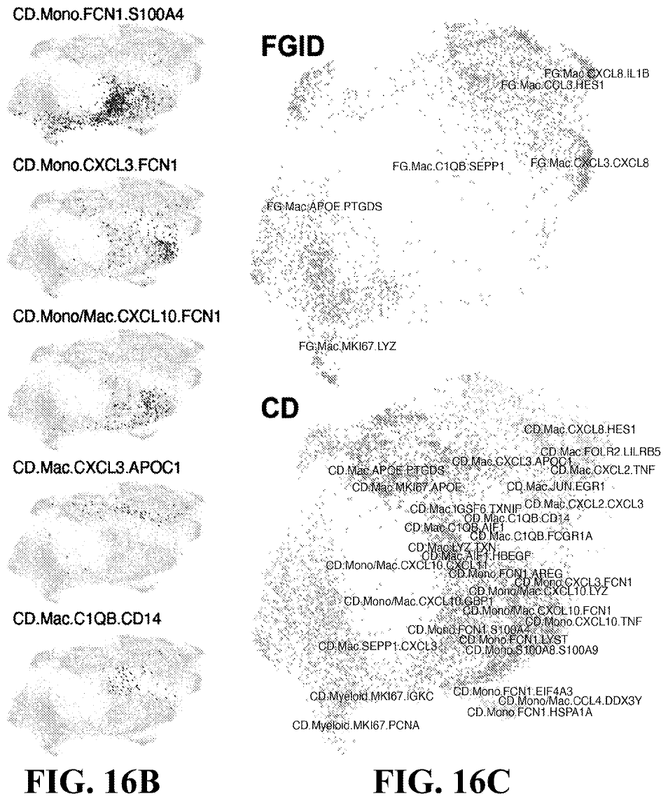

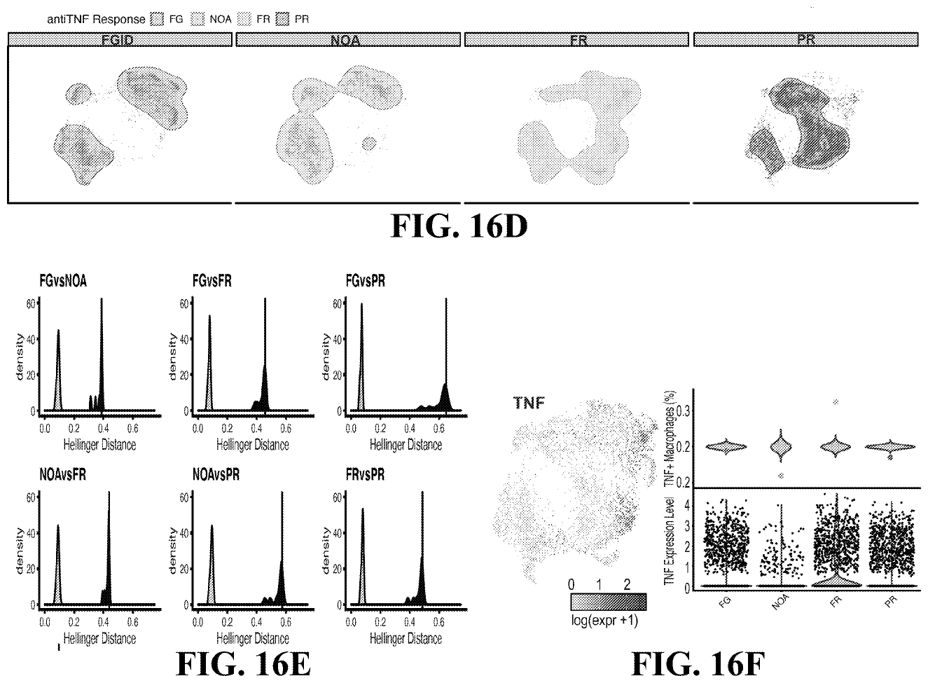

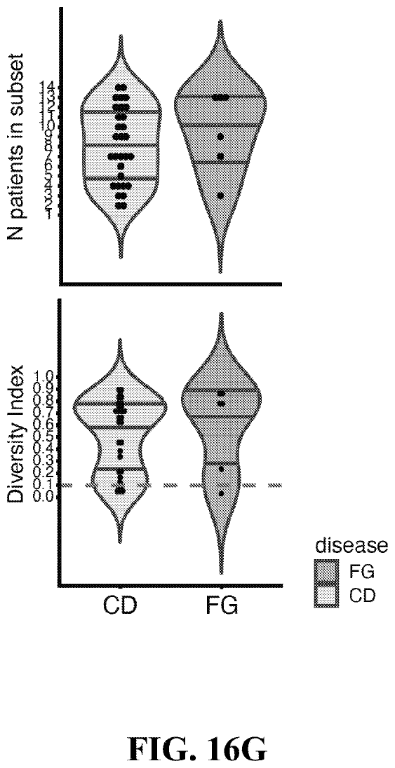

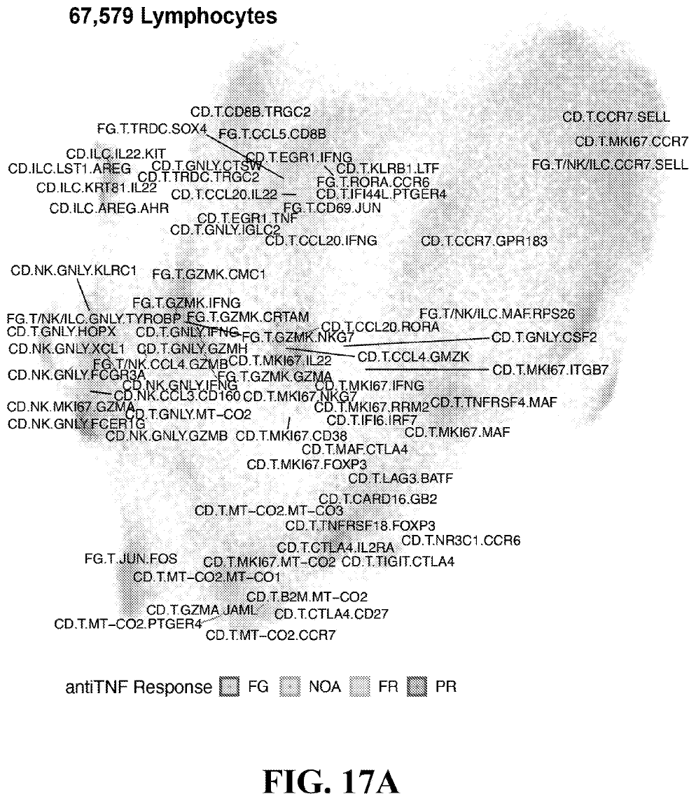

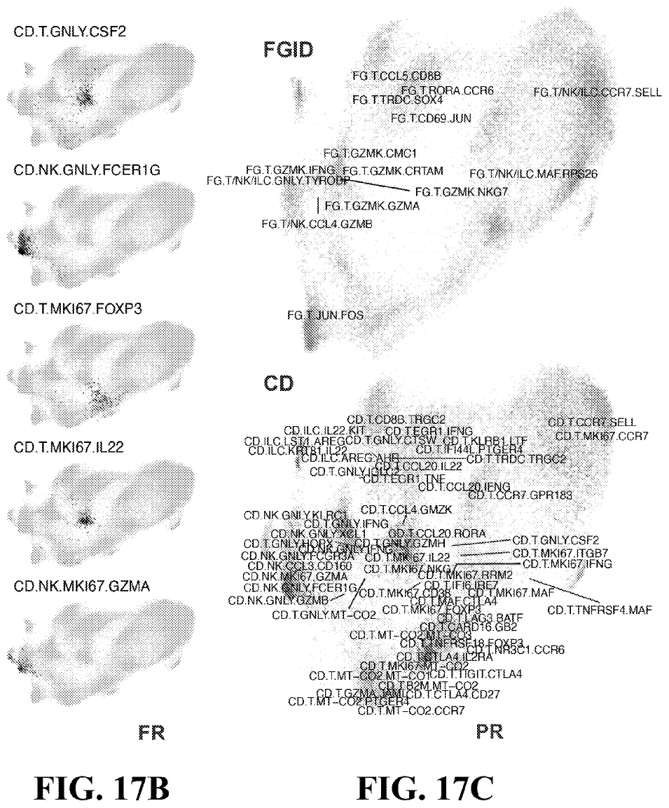

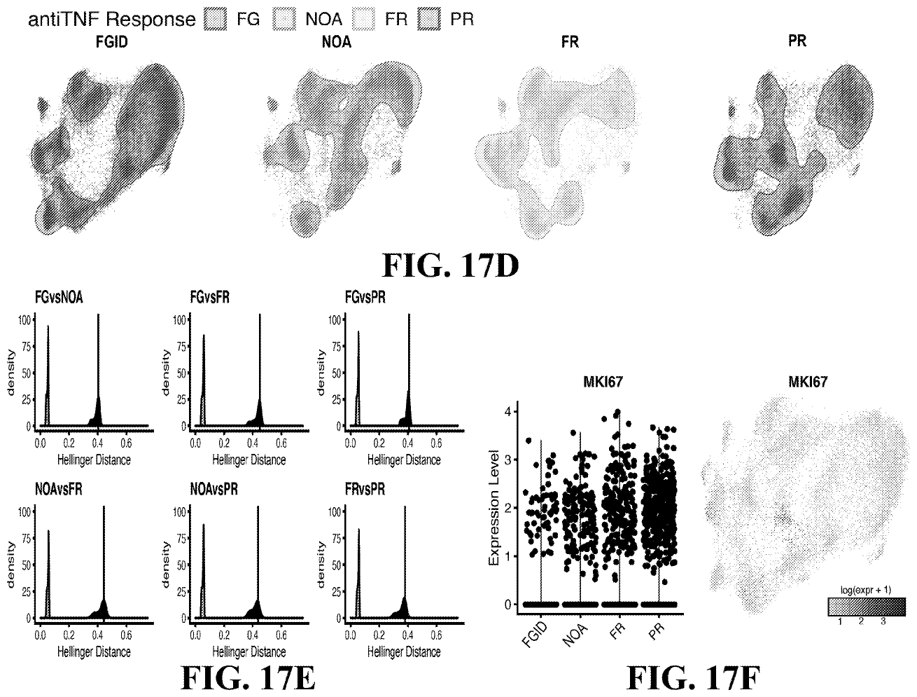

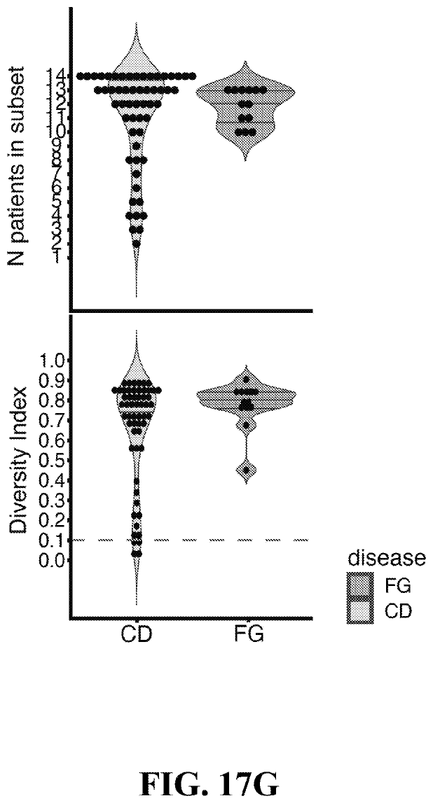

Based on their over-representation within clusters showing more significant differences within pediCD, Applicants next focused on performing an analysis over a shared gene expression space of FGID and pediCD of the monocytes/macrophages (FIG. 16) and T/NK/ILCs (FIG. 17). Applicants utilized a list of genes that were cell-type defining genes in either FGID or pediCD, but removed genes that were differentially expressed between FGID and pediCD, to allow for cell type/subset to drive placement on the UMAP (Methods) (Ordovas-Montanes et al., 2018). This allowed Applicants to place the fine-grained clusters within a joint gene-expression space related to underlying cell types in FGID and pediCD, and Applicants also contextualize the findings with an orthogonal integration approach applied to the T/NK/ILCs (FIG. 25) (Andreatta and Carmona, 2021).

Within FGID monocytes/macrophages, Applicants identified that the majority of clusters occupied the periphery of the UMAP space, including chemokine-expressing clusters (FG.Mac.CCL3.HES1; FG.Mac.CXCL8.IL1B) and metabolic clusters (FG.Mac.APOE.PTGDS) (FIG. 16a,c). This was in stark contrast to the pediCD monocytes/macrophages, where Applicants identified that now many of the clusters occupied the central region of the UMAP (FIG. 16a,c). Applicants highlight several of the clusters that are significantly different in frequency between the pediCD groups which were found in this central region (FIG. 16b). Notably, NOA, FR and PR pediCD clusters had significantly different distributions within this space (FIG. 16c-e). There was a progressive increase in the Hellinger Distance (Figure legend 16e: a measure of the distance between two distributions) of the distributions from FGID to NOA, NOA to FR, and NOA to PR. PRs had the most significantly different distribution relative to FGID. This was in large part driven by cells from both FRs and PRs that inhabit the central region, together with a loss of density in the chemokine-expressing macrophages from FGID through to PRs. Of note, the frequency of TNF+ macrophages and its expression level of TNF was significantly increased in FRs relative to all other groups (FIG. 16f; permutation test shuffling anti-TNF response variable, FR had significantly more TNF+ cells than expected by chance with p approximating 0). Despite the fine-grained tiered clustering approach used, the majority of clusters had high Simpson's Diversity indices representing cell states found in several patients (FIG. 16g).

Within T/NK/ILCs, Applicants identified that FGID cells were more uniformly mixed with the pediCD cells relative to monocytes/macrophages (FIG. 17a). FGID cells occupied naïve and quiescent states, showed some signatures of activation, and also specialization towards helper and cytotoxic states (FIG. 17a,c) (Sallusto et al., 1999). The most notable changes in the Hellinger Distance distribution occurred between FGID and FR, rather than between FGID and PR as may have been expected (FIG. 17d,e). Similar to the monocytes/macrophages, the main area which gained density with increased disease severity was the central region: characterized by proliferation of several clusters increased in frequency within FRs and PRs to anti-TNF including T cells such as CD.T.MKI67.FOXP3 and CD.T.MKI67.IL22 (FIG. 17b-d). Proliferation-associated gene signatures were also seen in extreme corners driven by CD.NK.MKI67.GZMA, and significantly increased from FGID through to PR (FIG. 17b,f). Intriguingly the only T cell clusters in pediCD with gini coefficient <0.1 are from pediCD patients (FIG. 17g). Taken together with several cell clusters associated with pediCD proliferation overlapping with existing areas found in FGID, this indicates activation and more extreme diversification from existing T cell states driving the T cell clustering that defined pediCD. This is distinct from the recruitment and failed differentiation towards homeostatic cell states between FGID and pediCD that Applicants discovered in monocytes/macrophage clusters. Both joint protections confirm and extend the RF predictions.

In order to provide tissue-scale context and understand the impact on other cell types for these anti-TNF response associated lymphocyte states, Applicants assessed the relationship of these proliferating T and NK cell clusters with epithelial and myeloid cells (FIG. 25c). Applicants found that CD.T.MKI67.FOXP3 were strongly positively associated with CD.Goblet.RETNLB.ITLN1 and CD.EC.NUPR1.LCN2 secretory cell states. Conversely, Applicants found that CD.NK.MKI67.GZMA were significantly negatively correlated with CD.EC.ADH1C.EDN1, CD.Mcell.CSRP2.SPIB, CD.EC.ADH1C.RPS4Y1, CD.EC.GSTA2.TMPRSS15, and CD.EpithStem.LINC00176.RPS4Y1. This indicates the potential for cytotoxic activity of the proliferating NK cells towards more homeostatic cell states of epithelial cells, and critically of intestinal stem cells, with increased disease severity.

### Example 19—Discussion

Applicants present two comprehensive cellular atlases of FGID and pediCD, and then identify correspondence between the two. Applicants generated complete gene lists for cell types (1 vs. rest across all cells), subsets (1 v. rest across all cells), and states (1 vs. rest within cell type). Applicants then focused on pediCD, and those cell states and gene expression which distinguish between disease severity and FRs vs. PRs (Table 1, 2, 3, and 14). The study addresses a critical unmet need in the fields of IBD and systems immunology: the creation of an atlas of newly-diagnosed and untreated diseased tissue, coupled with detailed clinical follow-up to link diagnostic cell types and states with disease trajectory. This is especially true for GI disease and others like it which afflict tissues that are not easily accessible without operative or endoscopic intervention, and where tissue-specific immune pathology dictates disease severity and trajectory. Likewise, cross-sectional studies, as have been the norm for most previous scRNA-seq studies of IBD, are not able to overlay disease trajectory and treatment response onto the topography of a complex multi-cellular atlas, thus limiting the mechanistic and predictive inferences that can be drawn from the generated atlas (Corridoni et al., 2020a, 2020b; Drokhlyansky et al., 2019; Elmentaite et al., 2020; Huang et al., 2019; Kinchen et al., 2018; Martin et al., 2019; Parikh et al., 2019; Smillie et al., 2019). Furthermore, mouse models of CD, and of IBD more broadly, may not be the most appropriate models for understanding treatment resistance in pediCD (Neurath, 2019). To surmount these limitations, Applicants created a prospective clinical study, and enrolled patients requiring a diagnostic biopsy for possible IBD, prior to diagnosis. This allowed Applicants to capture a tremendously valuable control group: those patients with FGID, who experience GI symptoms without evidence of GI inflammation or autoimmunity. These uninflamed controls served as a critical comparator to contextualize the evidence of immune pathology that Applicants observed in patients with pediCD. With these detailed clinical phenotypes as the foundation, Applicants developed an automated ITC algorithm for scRNA-seq data, ARBOL, which defines a vector of T cells, myeloid cells and epithelial cells that cleanly stratifies both Crohn's disease severity and response to treatment.

The availability of comprehensive clinical, flow cytometric and scRNA-seq data from patients with pediCD and from uninflamed FGID controls created an unprecedented opportunity for comparative atlas creation. Applicants took the opportunity to develop a methodical, unbiased, approach to cell state discovery, ARBOL (github.com/jo-m-lab/ARBOL). ARBOL iteratively explores axes of variation in scRNA-seq data by clustering and subclustering until variation between cells becomes noise. The philosophy of ARBOL is that every axis of variation could be biologically meaningful so each should be explored, and that axes of variation are relative to the comparative outgroup, meaning that similar cell states may arise at distinct tiers. Once every possibility is explored, curation and a statistical interrogation of resolution are used to collapse clusters into the elemental transcriptomes of the dataset. ARBOL inherently builds a tree of subclustering events. As data is separated by major axes of variation in each subset, later rounds capture less pronounced variables. This comes with some caveats: variation shared by all cell types (for example, cell cycle stage) can make up one of the major axes of variation in the first round of clustering. Cell types can split up at the beginning, so the same splitting of B and T cells, for example, may happen further down in separate branches. The resulting tree of clustering events (FIG. 22a) is therefore neither indicative of true distances between end clusters nor a tree of unique groupings. Applicants address this problem by calculation of a binary tree of manually and computationally curated end clusters. Using a standardized method of end cluster naming, which Applicants describe in ARBOL's tutorial (jo-m-lab.github.io/ARBOL/ARBOLtutorial.html), Applicants found the resulting binary tree assorted end clusters into appreciated cell types and subsets (FIGS. 12 and 13), and also reveals further previously unappreciated granularity that will serve as the foundation for future work into the cellular composition of the gastrointestinal tract.

One of the primary remaining challenges going forward will be to identify which clusters are truly patient-unique, or are simply patient-unique at the cohort size to which Applicants are currently limited to. Applicants calculate a diversity metric for each end cluster to highlight those which are largely conserved between patients, and provide complete cluster-defining gene lists for both FGID and pediCD at three levels of clustering. Applicants also provide links to the data visualization portal to enable cross-atlas comparisons: singlecell.broadinstitute.org/single_cell/study/SCP1422/predict-2021-paper-fgid and singlecell.broadinstitute.org/single_cell/study/SCP1423/predict-2021-paper-cd.

One of the chief advantages of enrolling pediCD patients at diagnosis, and prior to any therapeutic intervention, was that Applicants were able to relate their diagnostic immune landscape with disease trajectory. In the pediCD group, Applicants identified 3 clinical subgroups. The first distinction was made by treating physicians, and classified patients with milder versus more severe clinical disease characteristics at diagnosis. The milder patients were not placed on anti-TNF agents (NOA), while the more severe patients were treated with monoclonal antibodies that neutralize TNF including infliximab and adalimumab. The second distinction between patient groups could not be made at diagnosis, but rather, was based on clinical and biochemical response to anti-TNF agents. Thus, of those patients treated with anti-TNF therapeutics, some were FRs, and some were only PRs, with PRs requiring anti-TNF dose modifications and the addition of other agents, and with ongoing, uncontrolled disease signs and symptoms. While differences in ant-TNF pharmacokinetics have been partially implicated in the need to dose-escalate anti-TNF agents in some pediCD patients, the study identifies foundational differences in the immune state at diagnosis in PR patients compared to the NOA and FR subgroups (D'Haens and Deventer, 2021; Ordis et al., 2012; Yarur et al., 2016). While standard flow cytometry was not able to distinguish the immune phenotype of NOA versus treated patients, scRNA-seq identified significant differences. The contextualization of the scRNA-seq derived predictive cellular vector with two other treatment-naïve bulk RNA-seq studies of Crohn's disease, underscores the broader applicability of the findings (Kugathasan et al., 2017; Verstockt et al., 2019).

Applicants noted significant cell state changes at diagnosis underlying clinically-appreciated disease severity that impacted the clinical decision to treat or not to treat with anti-TNF agents. These occurred within multiple clusters of T, NK, fibroblast, epithelial, monocyte, macrophage, and dendritic cells. For anti-TNF response, very few clusters exhibited significantly differential composition between FR and PR individuals. This suggests that multiple collective changes in several cell types may conspire to lead to differences in treatment outcomes. Indeed, when Applicants jointly considered a cellular principal component vector comprising epithelial cells, T/NK/ILCs, and myeloid cells, Applicants identified several clusters that together could delineate the full spectrum of NOA, FR, and PR. This cellular vector indicated that multiple T cell subsets, NK cells, monocytes, macrophages, and epithelial cells were altered in disease. Intriguingly, by finely clustering each cell type, Applicants found that proliferating T and NK cells do not represent a uniform population, but rather reflect functional specialization capturing FOXP3, IFNG, IL22, and GZMA as cluster-defining genes. Enriched in NOA individuals were epithelial cells involved in chemosensation (Tuft.GNAT3.TRPM5) and absorption of metabolites (EC.GSTA3.TMPRSS15), as well as stem cells (Banerjee et al., 2020; von Moltke et al., 2016; Sido et al., 1998). That pediCD severity is not uniquely predicted by a singular cell subset or gene is reflective of the complex genetics and environmental factors that have been implicated, along with the rich literature that has found significant changes by histology, flow cytometry, or mass cytometry in CD relative to control tissue (Buisine et al., 2001; Leeb et al., 2003; Leonard et al., 1995; Lilja et al., 2000; Mitsialis et al., 2020; Müller et al., 1998; Souza et al., 1999; Stappenbeck and McGovern, 2017; Takayama et al., 2010). However, with the PREDICT study, Applicants have discovered precisely which changes in CD cellular composition come together to form a predictive vector for both disease severity and treatment response. Intriguingly, the quantification and visualization of this response vector predicted a later escalation of one of the patients (p022; who appeared as an outlier FR in FIG. 14d) from FR to PR, which occurred after the database lock in December of 2020.

When considering the relationships between T cells and NK cells along with epithelial cells, Applicants captured that proliferating cytotoxic NK cell subsets like CD.NK.MKI67.GZMA were significantly negatively correlated with critical metabolic and progenitor epithelial cell subsets in pediCD. Conversely, proliferating regulatory CD.T.MKI67.FOXP3 were positively associated with secretory epithelial cells in pediCD, but did not appear related to the decrease in metabolic or progenitor cells. How T cell-derived cytokines impact intestinal regeneration and differentiation has recently been the focus of several studies, but the relationship of these fine-grained T cell subsets with specific epithelial cell states observed in the human intestine remained unknown (Biton et al., 2018; Lindemans et al., 2015). This work suggests that in the context of the ileum impacted by CD that there is further complexity to understand, particularly as it pertains to cytotoxic NK cells and T cells and their impact on epithelial cell homeostasis and regeneration.

The mapping of these disease severity-associated cell networks identifies a host of new potential therapeutic targets for pediCD, for many of which there are clinical-stage therapeutics that could be investigated. These include CD40L-blocking antibodies, IL-22 agonists, and targeted anti-proliferation agents (Betts et al., 2017; Lindemans et al., 2015; Miura et al., 2021; Ramanujam et al., 2020; Sootome et al., 2020).. A case can also be built for targeting inflammatory cytokines such as IL-1, and for interrogating agents aimed at mucosal healing including new anti-GM-CSF antibodies, given that several prominent cell subsets marked by CSF2 were enriched in the PR patients (Ai et al., 2021; Aschenbrenner et al., 2021; Castro-Dopico et al., 2020; Mehta et al., 2020; Mitsialis et al., 2020; Muro and Mrowiec, 2015). This atlas therefore provides a rigorous evidence-based rationale for proposing new therapeutic interventions, as well as a mechanism for interrogating the impact of new agents on the longitudinal immune landscape of pediCD patients.

Recent work on COVID-19 has also highlighted the challenges faced by systems approaches to capture baseline cell states that predict disease trajectory (Kaczorowski et al., 2017; Lucas et al., 2020; Mathew et al., 2020; Schulte-Schrepping et al., 2020; Su et al., 2020). In a disease of known infectious etiology with SARS-CoV-2, monocytes, macrophages, granulocytes, T cells, B cells, antibodies, and interferon state have all independently been associated with disease outcomes. Few studies have considered how multiple collective changes at baseline may influence outcome, yet are likely more reflective of the disease. With the complex and protracted presentation of a multifactorial disease like Crohn's disease, Applicants posit that multiple concerted effects are required to dictate both the severity (NOA vs FR/PR) and the treatment-response (FR vs PR). Additional work can consider which cell subsets are recovered during mucosal healing, and how closely the treated state reflects each individual patient's baseline presentation.

### Example 20—Methods

### Study Population and Clinical Parameters

Pediatric patients less than 20 years of age with suspected inflammatory bowel disease were enrolled on the PREDICT Study (ClinicalTrials.gov #NCT03369353) Enrollment took place between Nov. 9, 2017 to Dec. 21, 2018 in accordance to an institutional review board approved protocol with written informed consent and assent when applicable. Patients diagnosed with Crohn's Disease (CD) were included and patients without gut inflammation on endoscopy and histology, and who were diagnosed with Functional GI Disease (FGID), served as a comparative cohort for this study. Terminal ileum and blood samples were taken during the diagnostic endoscopy procedures prior to initiation of therapy. Patients diagnosed with other inflammatory or infectious etiologies on endoscopy and biopsy were excluded from the analysis.

Clinical course and variables were monitored at the time of enrollment and for 3 years after initial endoscopy, with median follow up for CD being 32.5 months and FGID being 31 months at the time of clinical database lock (Dec. 1, 2020). Medical management was dictated by clinicians. Clinical variables obtained included sex, race, age at diagnosis, weight z-score, height z-score, BMI z-score, clinical disease severity using the Pediatric Crohn's Disease Activity Index (PCDAI), and disease location and phenotype using the Montreal Criteria (Hyams et al., 1991; Silverberg et al., 2005). Laboratory evaluation included C-reactive protein, ESR, hemoglobin, albumin, white blood cell count, and platelet count.

### Response to Anti-TNF Therapy

Early anti-TNF or immunomodulator therapy was defined as initiation of immunosuppression within 90 days of diagnostic endoscopy. Anti-TNF monoclonal antibody was started in 10 patients with CD. All patients were followed prospectively and categorized as full responders (FR), partial responders (PR), or not on anti-TNF (NOA). Full response to anti-TNF is defined as clinical symptom control and biochemical response with wPCDAI score of <12.5 on maintenance anti-TNF therapy and partial response defined as lack of clinical symptom control and biochemical response with documented escalation of anti-TNF therapy.

### Clinical Statistical Analysis

Clinical variables are expressed as median (lower and upper confidence interval; range) and compared using the Mann-Whitney U test. Categorical variables were described as frequencies and percentages and compared using the chi-square test. Clinical laboratory values are represented by mean and standard error of the mean (range) and compared with the Mann-Whitney U test. Significance is indicated by a P value of <0.05. Clinical statistical analysis was performed using GraphPad Prism version 8.3.0.

Tissue Dissociation into Single-Cell Suspensions

Human Ileum. Single-cell suspensions were collected from intestinal biopsies using a modified version of a previously published protocol (Persson et al., 2013) as described in (Smillie et al., 2019). One biopsy from the terminal ileum was received directly in hand and processed with an average time from patient to loading on the 10× Chromium platform of 2.5 total hours, and never exceeding 3.5 hours. While intact, biopsy bites were handled using a P1000 pipette applying gentle suction, and all centrifugation steps done in a temperature controlled 4° C. centrifuge. Biopsy bites were first rinsed in 30 mL of ice-cold PBS (ThermoFisher 10010-049) and allowed to settle. Each individual bite was then transferred to 10 mL epithelial cell solution (HBSS Ca/Mg-Free [ThermoFisher 14175-103], 10 mM EDTA [ThermoFisher AM9261], 100 U/ml penicillin [ThermoFisher 15140-122], 100 μg/mL streptomycin [ThermoFisher 15140-122], 10 mM HEPES [ThermoFisher 15630-080], and 2% FCS [ThermoFisher 10082-147]) freshly supplemented with 200 μL of 0.5M EDTA. Separation of the epithelial layer from the underlying lamina propria was performed for 15 minutes at 37° C. with rotation at 120RPM. The tube was then removed and placed on ice immediately for 10 minutes before shaking vigorously 15 times. Visual macroscopic inspection of the tube at this point yielded visible epithelial sheets, and microscopic examination confirmed the presence of single-layer sheets and crypt-like structures.

The remnant tissue bite was carefully removed and placed into a large volume of ice-cold PBS to rinse before transferring to 5 mL of enzymatic digestion mix (Base: RPMI1640, 100 U/ml penicillin [ThermoFisher 15140-122], 100 μg/mL streptomycin [ThermoFisher 15140-122], 10 mM HEPES [ThermoFisher 15630-080], 2% FCS [ThermoFisher 10082-147], & 50 μg/mL gentamicin [ThermoFisher 15750-060]), freshly supplement immediately before with 100 μg/mL of Liberase TM [Roche 5401127001] and 100 μg/mL of DNase I [Roche 10104159001]), at 37° C. with 120 rpm rotation for 30 minutes. During this 30-minute lamina propria (LP) digestion, the epithelial (EPI) fraction was spun down at 400 g for 7 minutes and resuspended in 1 mL of epithelial cell solution before transferring to a 1.5 mL Eppendorf tube in order to minimize time spent centrifuging and provide a more concentrated cell pellet. Cells were spun down at 800 g for 2 minutes and resuspended in TrypLE express enzyme [ThermoFisher 12604013] for 5 minutes in a 37° C. bath followed by gentle trituration with a P1000 pipette. Cells were spun down at 800 g for 2 minutes and resuspended in ACK lysis buffer [ThermoFisher A1049201] for 3 minutes on ice to remove red blood cells, even if no RBC contamination was visibly observed in order to maintain consistency across samples. Cells were spun down at 800 g for 2 minutes and resuspended in 1 mL of epithelial cell solution and placed on ice for 3 minutes before triturating with a P1000 pipette and filtering into a new Eppendorf tube through a 40 μM cell strainer [Falcon/VWR 21008-949]. Cells were spun down at 800 g for 2 minutes and then resuspended in 200 μL of epithelial cell solution and placed on ice while final steps of LP dissociation occurred. After 30 minutes, the LP enzymatic dissociation was quenched by addition of 1 ml of 100% FCS [ThermoFisher 10082-147] and 80 μL of 0.5M EDTA and placing on ice for five minutes. Samples were typically fully dissociated at this step and after gentle trituration with a P1000 pipette filtered through a 40 M cell strainer into a new 50 mL conical tube and rinsed with PBS to 30 mL total volume. This tube was spun down at 400 g for 10 minutes and resuspended in 1 mL of ACK and placed on ice for 3 minutes. LP cells were spun down at 800 g for 2 minutes and resuspended in 1 mL of epithelial cell solution and spun down at 800 g for 2 minutes and resuspended in 200 μL of epithelial cell solution and placed on ice. Following centrifugation, the cells from both EPI and LP fractions were counted and prepared as a single-cell suspension for scRNA-seq. Since the full EPI isolation was not performed on all patients limiting sample sizes, here Applicants focus the analysis on LP fractions.

### Flow Cytometry

Multicolor flow cytometry was performed on tissue samples to examine the immune composition for enrolled patients. Flowjo software was used to phenotypically define cell populations that will be analyzed and compared in patients using two-way ANOVAs (or non-parametric equivalent). Antibodies used include: CD3 APC, SP34-2 (BD Biosciences); CD3 BUV661, UCHT1 (BD Biosciences); CD3 BV711, OKT3, (Biolegend); CD3 PE, SP34 (BD Biosciences); CD4 BV785, OKT4 (Biolegend); CD8a BUV395, RPA-T8 (BD Biosciences); CD8b FITC, REA715 (Miltenyi Biotec); CD11b APC-Cy7, ICRF44 (BD Biosciences); CD11c APC-eFlour 780, BU15 (Fisher Scientific); CD11c BUV661, B-ly6 (BD Biosciences); CD14 APC-eFluor 780, 61D3 (Fisher Scientific); CD14 BUV737, M5E2 (BD Biosciences); CD20 APC-eFluor 780, 2H7 (Fisher Scientific); CD20 PE-Cy7, L27 (BD Biosciences); CD38 APC, HIT2 (BD Biosciences); CD45 PerCP/Cy5.5, HI30 (Biolegend); CD45RA BV605, HI100 (Biolegend); CD56 (NCAM) FITC, TULY56 (Fisher); CD94 APC-Vio770, REA113 (Miltenyi Biotec); CD117 (c-kit) BV421, 104D2 (Biolegend); CD123 BV711, 9F5 (BD Biosciences); CD127 Biotin, HIL-7R-M21 (BD Biosciences); CD161 BV711, DX12 (BD Biosciences); CD197 (CCR7) BV421, G043H7 (Biolegend); CD294 (CRTH2) BV605, BM16 (Biolegend); CD326 (Epcam) APC, HEA-125 (Miltenyi Biotec); HLA-DR APC-H7, L243 (G46-6) (BD Biosciences); TCR PAN γδ PE-Cy7, IMMU510 (Beckman Coulter); α4-β7 integrin (Act-1), (NIH AIDS Reagent Program); Streptavidin BUV737 (Fisher); Live/dead Fix Aqua (Fisher); R-PE Antibody Labeling Kit (300 mcg) (Abcam).

### Methods to Generate Single-Cell RNA-Seq Libraries and Sequencing

10× v2 3′. Single cells were loaded onto 3′ library chips as per the manufacturers protocol for Chromium Single Cell 3′ Library (v2) (10× Genomics). The LP fraction was captured in its own channel of the 10× Chromium Single Cell Platform, in order to recover sufficient numbers of cells for downstream analyses. An input of 10,000 single cells was added to each channel with a recovery rate of 9,514 cells per sample based on median across samples. Briefly, single cells were portioned into Gel Beads in Emulsion (GEMs) in the Chromium controller with cell lysis and barcoded reverse transcription of RNA, followed by cDNA amplification, enzymatic fragmentation and 5′ adaptor and sample index attachment. Libraries were sequenced on a HiSeq or NovaSeq flow cell. The read structure was paired end with length of read 1 26 bp, length of read 2 91 bp, and the length if index 1 (i7 primer) 8 bp. Quality-filtered base calls were converted to demultiplexed FASTQ files.

### Alignment and Filtering.

FASTQ files were aligned to GRCh38 using Cellranger v2.2 pipeline on the Cumulus/Terra cloud pipeline portal.firecloud.org/?retum=firecloud #methods/cumulus/cellranger_workflow/10 generating 27 cell-by-gene matrices (13 FGID, 14 CD), one for each patient. Applicants used default parameters of the 10^{th} snapshot version of the pipeline, aside from requiring that it use cellranger v2.2.0.

Every sample was first filtered excluding genes measured in fewer than 3 cells and cells with fewer than 200 unique genes. To control for doublets and low-quality cells Applicants then further filtered individually, attempting to match the approximate 10,000 cells loaded onto the sample lane and balancing the thresholds to not cut out dense regions of a Ncounts by Nfeatures scatter plot. Pre-filtering, Applicants looked for outlier samples, based on proportion of percent mitochondrial genes, number of counts, and number of features, none fell beyond the 1.5 times the IQR threshold.

Exact thresholds used for each sample:

| Sample ID | Diagnosis | min_cells | min_genes | max_genes | max_count | N_cells_post |
|---|---|---|---|---|---|---|
| p011_TOD_ILE_LPS_3p | CD | 3 | 200 | 3000 | 20000 | 11087 |
| p014_TOD_ILE_LPS_3p | CD | 3 | 200 | 3000 | 20000 | 7285 |
| p016_TOD_ILE_LPS_3p | CD | 3 | 200 | 3000 | 20000 | 7713 |
| p018_TOD_ILE_LPS_3p | CD | 3 | 300 | 4000 | 20000 | 15469 |
| p019_TOD_ILE_LPS_3p | CD | 3 | 200 | 5000 | 20000 | 11200 |
| p022_TOD_ILE_LPS_3p | CD | 3 | 200 | 3000 | 20000 | 7798 |
| p024_TOD_ILE_LPS_3p | CD | 3 | 200 | 3000 | 20000 | 9705 |
| p026_TOD_ILE_LPS_3p | CD | 3 | 300 | 4000 | 30000 | 10847 |
| p027_TOD_ILE_LPS_3p | CD | 3 | 200 | 4000 | 40000 | 8124 |
| p031_TOD_ILE_LPS_3p | CD | 3 | 200 | 3000 | 20000 | 12177 |
| p032_TOD_ILE_LPS_3p | CD | 3 | 200 | 3000 | 20000 | 9052 |
| p041_TOD_ILE_LPS_3p | CD | 3 | 200 | 4000 | 40000 | 7508 |
| p042_TOD_ILE_LPS_3p | CD | 3 | 200 | 4000 | 30000 | 12219 |
| p048_TOD_ILE_LPS_3p | CD | 3 | 200 | 4000 | 40000 | 9158 |
| p009_TOD_ILE_LPS_3p | FGID | 3 | 200 | 3000 | 20000 | 11426 |
| p017_TOD_ILE_LPS_3p | FGID | 3 | 200 | 3000 | 20000 | 11107 |
| p023_TOD_ILE_LPS_3p | FGID | 3 | 200 | 3000 | 20000 | 11262 |
| p028_TOD_ILE_LPS_3p | FGID | 3 | 200 | 4000 | 20000 | 10293 |
| p030_TOD_ILE_LPS_3p | FGID | 3 | 200 | 4000 | 30000 | 9590 |
| p033_TOD_ILE_LPS_3p | FGID | 3 | 200 | 3000 | 20000 | 9514 |
| p034_TOD_ILE_LPS_3p | FGID | 3 | 200 | 3000 | 20000 | 8142 |
| p035_TOD_ILE_LPS_3p | FGID | 3 | 200 | 3000 | 20000 | 5668 |
| p037_TOD_ILE_LPS_3p | FGID | 3 | 200 | 3000 | 20000 | 4210 |
| p043_TOD_ILE_LPS_3p | FGID | 3 | 200 | 5000 | 30000 | 5045 |
| p044_TOD_ILE_LPS_3p | FGID | 3 | 350 | 4000 | 30000 | 12727 |
| p049_TOD_ILE_LPS_3p | FGID | 3 | 200 | 3000 | 20000 | 8003 |
| p050_TOD_ILE_LPS_3p | FGID | 3 | 200 | 4000 | 30000 | 8582 |

Post filtering, Applicants merged sample matrices using an outer join to create an FGID dataset (115,569 cells) and a CD dataset (139,342 cells).

### Quantification and Statistical Analysis

Preprocessing & Clustering of scRNA-Seq Data

### 1^{st} Approach Classical Methods on Combined Dataset

Applicants began initial analysis following traditional clustering and annotation techniques; however, these methods using manual and at times subjective metrics scaled poorly to the size and scope of the dataset and moreover did not give clear distinction between disease specific cell states and compositional shifts within cell states across disease.

For the first pass at analysis, Applicants grouped the FGID and CD datasets together (254,911 cells) and proceeded with the standard Seurat v3.1.5 pipeline (Stuart et al., 2019). Applicants used manual heuristics of gene marker specificity to choose cluster resolution and isolate 9 major cell types (T, B, plasma, epithelial, endothelial, fibroblast, myeloid, mast cell, and glial) and 1 aggregate cluster of T, B, myeloid, and epithelial cells with a strong proliferation signature. Applicants then subclustered the proliferating group and manually merged the proliferating cells with their corresponding cell type based on marker gene expression, and separately re-preprocessed and clustered each cell type annotating based on one vs. rest differential expression (Wilcoxon, fdr<0.05) within the cell type.

Applicants found several disadvantages to this approach. First, Applicants found it difficult to determine for each cluster whether Applicants should be looking for changes in compositional frequency or gene expression. Particularly within the myeloid major cell group Applicants would find extremely disease biased sub-clusters, as much as a 9:1 ratio between CD to FGID. It was unclear whether there was massive compositional shift within a conserved cell state or if instead a base cell state was split into multiple clusters based phenotypic differences in disease and Applicants should perform a differential expression test between it and neighboring FGID biased clusters. Second, after two rounds of manual processing Applicants were still unsure if Applicants had reached a base level with each end cluster corresponding to a unique and biologically homogeneous cell state. Third, at that point, having partitioned over 100 distinct clusters, individually supervising each subsets processing and sub-clustering was infeasible. Applicants needed a more systematic method to address these challenges.

### 2^{nd} Approach Automated Hierarchical Tiered Clustering on Separated Disease Conditions

It is common to organize cell identity ontologies in a tree structure. With major groups such as immune, stromal, and epithelial at the top and branching down a level, you might set more nuanced identities like T, B, endothelial and goblet cell types as a second tier, and even more nuanced identities like CD4+ vs CD8+ T cells as a third. In ideal circumstances, this mental model conforms well to RNA-seq data where Applicants can layer gene modules with more and more specific variation together to describe highly particular cell identities and states. And, by clustering at a high level with genes that vary across the entire dataset, then sub-clustering with genes that vary only within a particular parent cluster Applicants are able uncover this hierarchy of cell identity. Reality is of course much messier than theory and many additional factors to cell identity contribute to the variation in gene expression within actual datasets, particularly as Applicants found with disease condition during the first approach.

To be able to choose the appropriate future analyses and comparisons, Applicants need a highly accurate representation of cell identity and state. The underlying issue in the first pass at clustering was that in combining the disease conditions together, the variable genes selected at each stage represented a combination of differences between cell identity & disease. This combination could have been manageable if either disease or cell identity were consistently more variable. Applicants could isolate one factor at a specific tier in the hierarchy before sub-clustering to isolate the other. In the case, disease and cell identity both had many overlapping scales of variation. To address this problem, Applicants isolated cell identity by separating the dataset by disease and clustering for cell identity within each disease set (FGID 115,569 cells, CD 139,342 cells). This approach did then require Applicants to perform an additional stage of analysis to find corresponding clusters between the two datasets, but allowed for far more effectively distinguishing type, scale, and specificity of disease differences.

Within each disease set Applicants still needed a method to ensure Applicants were reaching the bottom level of biological heterogeneity, and preferably an automated method as the first pass had shown the potential for isolating hundreds of cell states. To efficiently cluster and isolate these cell states Applicants wrote a cloud-based pipeline to systematically optimize parameter selection and stop when biological heterogeneity is exhausted. Homogenous cell subsets were isolated by recursively normalizing, selecting variable genes, and clustering based on silhouette score. Applicants stopped recursing into sub-clusters once Applicants reached one of four end conditions defined as:

Having a group of less than 100 cells (though Applicants did partition many clusters smaller than 100 cells after clustering groups just larger than that cutoff).

Isolating an optimized clustering of only one cluster.

Finding two clusters that have fewer than 25 genes (fdr<0.05 & |log fold change|>1.5 & percent expression>=20% in at least one cluster) differentially upregulated between each cluster using a bimodal test developed in (Shekhar, 2016 10.1016/j.cell.2016.07.054). For this last condition, if reached, Applicants reject the clustering and return back the cells as a single end cluster.

Having reached a max tier limit. Setting this value to 10, Applicants never triggered this condition with either FGID or CD datasets, but included it to prevent runaway recursion.

Code for generating this tree of cell clusters is currently available here: (jo-m-lab.github.io/ARBOL/ARBOLtutorial.html). Within each recursion, the established steps were processed using Seurat version 3.1.5 (github.com/satija.lab/seurat). Normalization and variable gene selection were processed with SCTransform (github.com/ChristophH/sctransform) (Hafemeister and Satija, 2019). Clustering for major cell types was performed using Louvain clustering on dimensionally reduced principal components.

Parameters depend on the size of the dataset, and thus must be adjusted based on how many cells are being partitioned for each recursive step. When calculating nearest neighbor graphs, and clustering Applicants set the K parameter to ‘ceiling(0.5*sgrt(N))’. Applicants chose the number of principal components based on the top 15 percentile of calculated improvement of variance explained. For subsets less than 500 cells Applicants used Jackstraw to calculate significant principle components. If neither method succeeded, Applicants chose the first two principle components. Applicants set clustering resolution via a grid search optimizing for maximum average silhouette score, (Silhouette measures the ratio of intra-cluster distance to inter-cluster distance, where a high score means highly distinct clusters). For stages where Applicants were clustering more than 500 cells a randomized subsample of N cells/10 was used to calculate the average silhouette score.

Additionally, at each recursive step Applicants output quality metrics and basic plots, such as 1:rest differential expression from the optimal partitioning at each stage and UMAP representations painted by sample metadata (sample ID, cluster number). The pipeline, saved output as a directory structure matching the tree discovered by this recursive clustering. This tree represents the lower levels of variance of discovered at each tier. At any tier level Applicants are able to extract the cell's partitioning. Due to the intermixing of patient and cell identity effects at multiple levels of the tree (a fraction of a single patient's cells might separate out at a high level, but then continue to separate into identifiable cell types, or vice versa), Applicants found the most meaningful levels at the top and bottom of the tree. The clustering tree is useful for understanding the levels of variance in the dataset, but Applicants found it contains too much noise to be easily interpretable. Thus, Applicants later generated a hierarchical clustering of the bottom level clusters based on pairwise differential expression, which is displayed in figures (FIG. 12: FGID atlas, FIG. 13: pediCD atlas). See the hierarchical clustering of cell subsets section for details.

Cell Type and Subset Annotation from Tiered Clustering

After running the hierarchical tiered clustering pipeline Applicants manually curated the generated tree of clusters. Specifically, tree generation was reinitiated for the B Cells within the FGID dataset as it had stopped at the first tier on two clusters with <50 genes differentially expressed, however Applicants could see in this case that there was additional biological stratification based on strong differential expression of CXCR4 (Wilcoxon; log FC=1.22860917, Bonferroni.p=1.0E-300) CD69 (Wilcoxon; log FC=1.27527652, Bonferroni.p=2.99E-151), HMGN1 (Wilcoxon; log FC=−1.1688612, Bonferroni.p=1.62E-227) and HMGA1 (Wilcoxon; log FC=−1.28838294, Bonferroni.p=1.06E-209) among others. This formed a clear divide between non-proliferating and proliferating B Cells, further validated by a clear separation within the UMAP based on PCA reduced variable genes within the B cells. Applicants further examined each branching point of the tree to determine its splitting cause, noting splits based on spillover, doublet, and singular patient effects. Splits at higher tiers based on doublets often split again allowing Applicants to recover cells that did not have the dual expression profile. Splits that only had patient splits below (measured by having only clusters of single patients) were manually marked as end clusters, thereby merging all clusters below that split. With these manual steps made, Applicants performed pairwise differential expression to ensure each partitioned subset is distinct from its neighbors.

Applicants annotated these final clusters with four methods attempting to balance descriptiveness, ease of understanding, and ease of name generation: The first method, is generated during the hierarchical tiered clustering by following the path from the end cluster up to the original tier. An example annotation is T0C0.T1C3.T2C3.T3C5 marking an end cluster that split at tier 1 into cluster 0 and at tier 2 into cluster 3. These annotations do not provide any biological information to the reader, but do provide a unique ID for the end cluster. The second method is far more descriptive, where Applicants manually annotate the main reason for each particular split. This still follows the original ranking of variation as found by the hierarchical tiered clustering, while also providing biological interpretation, as an example: CD.Mloid.macrophage_chemokine.S100A8 S100A9_CXCL9_CXCL10_TNF_inflamonocyte.

This method of annotation was particularly useful during analysis as Applicants were immediately able to see how early or late two clusters had split from each other, as well as seeing a number of the subset defining genes. Unfortunately, as is apparent this method also produces extremely long names that are difficult to display and refer to. It is also a highly manual process, and difficult to reproduce precisely. To better present the findings and aid others in reproducing the results, the third method automates this annotation. This method is performed by taking each major cell type, which in the case matched the tier one splits, and performing 1:rest differential expression testing (Wilcoxon; adj.p<0.05, only.pos=True) within each major cell type. Applicants then ranked the genes based on the product of ‘−log(Bonferroni.p)’, ‘avg_logFC’, and ‘pct.exp.1/pct.exp.rest’ and took the top 5, forming a name like CD.Mloid_CCL3_CCL4_CCL3L3_TNF_TNFAIP6. This scheme, again was useful, but did not quite meet the demands of recognizability and brevity. Thus for T and Myeloid cells Applicants adjusted these names to a finer degree of specificity by visualizing the expression profiles of each subset with a dotplot of canonical marker genes based off of current literature, and limiting to the top 2 genes based off the method 3 rankings and the dotplot of canonical markers, thereby producing the fourth and final annotations in the form: CD.Mono.CXCL10. TNF. Due to the limited nature of current characterization of stromal and epithelial cells Applicants were unable to match the same degree of specificity as the T and Myeloid cells, however Applicants did where possible adjust from the major cell type, to the most specific that Applicants could be confident of. For instance, adjusting “Epith” to “Goblet” based on marker expression of TFF3 and MUCN13.

### Hierarchical Clustering of Subsets on Unified Gene Space and Removal of Doublets

At this point Applicants had generated a hierarchical representation of the datasets from the top down showing the splits of highest variation at every level. By necessity that means that each level is controlled by and represents different selections of genes, which may have no relation to the genes selected in another branch. To understand the relations of cell subsets and compare across cell type Applicants needed a unified set of genes. For each dataset (FGID and CD) Applicants performed pairwise differential expression (Wilcoxon; Bonferroni.p<0.01) and selected the top 50 most significant genes from each test. Gene lists were merged as a union, finding 4445 unique genes for FGID and 1760 unique genes for CD that best differentiate the subsets. Subset centers were calculated from these selected genes as the median expression of cells grouped by subset. The resulting table was then hierarchically clustered using correlation distance and complete linkage. Clustering was performed in R using the pvclust package (github.com/cran/pvclust)

The resulting tree shows from the bottom up the relationships between cell subsets, and allows cell subsets that were potentially misclassified at a high split in hierarchical tiered clustering to find their biological neighboring subsets. As previously mentioned within the description of hierarchical tiered clustering Applicants did not find any end cluster subsets that met the thresholds for merging. This does not mean that Applicants did not observe shuffling from the initial tiered splits. While overall there was good agreement between the two methods, Applicants noted subsets jumping between major cell types as defined by the first splits of the tiered clustering. Applicants identified the majority of these jumping subsets as doublet clusters by exploring their differential gene results at multiple levels of the tiered clustering tree. Applicants removed these doublet subsets and others based on flipping expression programs at different tiers. For instance, looking like T cells expressing TRAC, IL7R within an epithelial cluster, than at the next tier expressing KRT18 and PIGR. After removing doublets, Applicants recalculated subset distances and dimensional reductions, as presented in the main figures.

### Finding Corresponding Cell Subsets Between Disease

Separating the data on disease condition into two datasets was important as it allowed Applicants to isolate the axis of cell identity within each disease and be confident in the homogeneity of each subset.

### KNN Classifier

The first attempt to find corresponding clusters followed the methods of Tasic et al. 2016. Applicants used the best differentiating genes sets created for the unified gene space clustering to as the mapping space for a nearest-neighbor classifier. For each cell within the a disease condition, Applicants could map it to the nearest cell subset within the other disease condition. As a trial run Applicants created this gene space for each major cell type of the FGID disease condition and performed 5-fold cross-validation.

Applicants further used an automated system to choose genes as the most significantly differentially expressed genes in order to create enough separation between cluster centers to effectively classify new cells. Applicants chose to use a random forest classifier as it allowed Applicants to train for the optimal selection of genes, required little to no preparation of data, and provided probabilities of each cell being predicted to each class. These probabilities for each class proved particularly useful do to the second realization. Because the number of subsets differs between disease conditions, Applicants cannot make the assumption that there is a one to one relationship between conditions. Applicants also cannot make the assumption that the many to one relationships are unidirectional with one base subset splitting into many states only from FGID toward CD. A single classifier would not allow Applicants to distinguish between these many types of relationships. However, Applicants realized that by creating a classifier for both directions (FGID to pediCD and pediCD to FGID) Applicants could take advantage of the difference in confidence between the two classifications to discover the direction and type of relationship. For 1:1 relationships, Applicants would expect all cells of subset A in condition X to match with 100% confidence to subset A in condition Y. In that particular case the summed probability equal 2 and there would be zero difference in confidence of one classifier to its matching classifier. For non-1:1 relationships, Applicants might instead see 90% of cells of subset A in condition X to matching with >85% confidence to subset B in condition Y, and only 30% of subset B in condition Y matching with >85% confidence. From this discrepancy Applicants can to infer that subset A may be a cell state in condition X that is layered on top of a base state B in condition Y. Low confidence in both directions indicates subsets unique to a particular condition.

### Training Random Forest Model

After these realizations Applicants trained random forest classifiers for each cell type in each disease condition using SciKit-Learn v0.22.2, with the intent to classify each cell to the subset in the opposed dataset the cell is most similar to (Pedregosa et al., 2011). For each cell type Applicants optimized a classifier for accuracy using grid-based search tuning number of trees, depth, number of features, criterion, and min samples per leaf with 5-fold cross validation for each set of tuning parameters. Applicants never observed full overfitting where the accuracy on test folds began to drop with increased size of model, but Applicants did quickly find diminishing returns as Applicants increased model size. For simplicity and because optimal tuning parameters were robust to overfitting, Applicants chose to use the same largest model parameters for all models (number of trees=500, depth=200, number of features=sqrt, criterion=gini, min samples per leaf=1). The initial training rounds found accuracies in the mid 60%. A definite increase from the NN classifier, but not high enough for Applicants to be confident in the results. The main issue Applicants eventually determined to be the uneven class distributions (far more cells in subset A than subset B). This caused the smaller subsets to be under trained. To compensate Applicants up-sampled with replacement each subset within the training fold to contain at least the 75th quantile number of cells. This single change improved accuracy on the unmodified test fold the most, varying from 5-15% improvement of accuracy, precision and recall across each cell type and provided accuracies ranging from high 70 to low 90 percent per major cell type.

### Applying Random Forest Model

Applicants ran the random forest model across the disease conditions. Applicants trained each random forest model with optimized parameters on all folds of its dataset, then proceeded to get probability predictions for each cell from the disease condition to the trained disease condition. With these class probabilities per cell Applicants could aggregate for each disease condition by taking the mean class probabilities for each group, leaving Applicants with 2 n by m table where n equals the number of subset groups and m equals the number of subset classes in the opposing disease condition. Using the mean probabilities for the group allowed more information from the cell level to rise to the aggregated levels than using the individual class prediction alone (computed as the class with max confidence of cell membership). These tables also provide confidences to all classes which is important for understanding the transverse confidence in both directions.

It is especially important to understand the many one to one relationships between disease conditions and find where a base cell state becomes layered in additional expression profiles, as these are the exact cases where Applicants can infer the underlying signaling patterns that diversify or concentrate cell state profiles. In diverse splitting of a subset across disease Applicants can start to understand the heterogeneity of patient response to treatment as it becomes clear which particular cell profiles are correlated with strong and poor response. To gain insight to these changes, Applicants care about where there is strong confidence in both directions and where there is strong confidence in only one direction. The simplest method to calculate these is to separately take the sum of the pairwise prediction confidences and the difference. Applicants call the sum of confidences the correspondence of a subset, and the difference the bias.

### Visualizing Correspondence and Bias

Applicants plot these metrics on a dot plot where each possible connection is laid out on a grid. For each dot Applicants set the size to match the correspondence, and color the dot based on the bias, such that a perfect match would appear as a large white circle. A more unidirectional match would be tinted darker in the color matching the disease condition with more confidence. Matches with more bias tend to indicate a subset matching a base cell state but also expressing some additional gene modules. To aid the human eye on picking up the major patterns Applicants filter to only show the top 10% highest correspondences. This parameter was chosen after looking at the distribution of correspondence scores and selecting the majority of the right tail of the distribution. It keeps the strongest matches in both ways and keeps the strongest in highly biased matches. To also aid the human eye Applicants perform a hierarchical clustering using cosine distance and complete linkage on the prediction confidences and compute an optimal ordering based on the cosine distances using the “cba” package in R: cran.r-project.org/web/packages/cba/index.html. This allows Applicants to sort subsets on the rows and columns such that subsets that get predicted similarly are next to each other. From this visualization Applicants are able to easily discern which are the subsets FGID that split into many phenotypes within CD from high correspondence and bias, which subsets don't change phenotype much at all based on high correspondence and low bias, and which are the subsets are potentially unique to a disease condition based on very low correspondence and bias.

### Association of Cell Subsets to Anti-TNF Response

Compositional differences are an important metric for understanding the baseline differences that prognose a patent's response to treatment. Applicants measure these differences with proportional enrichment of particular cell subsets within each patient, and finding the significantly reproducible enrichments across disease. As an extreme example Applicants might find that subset A cells comprise as much 80% of cells sampled in one condition whereas they might only comprise 30% in a different condition. This type of compositional analysis is highly affected by the number and choice of subsets included, and the sampling depth per patient (how many cells are collected). The first factor is controlled by the confidence in the clustering and using computationally optimized parameters. Applicants further control this factor by limiting analysis of compositional shifts of cell states to within major cell types. This isolates the chance of error from affecting the entire analysis and allows Applicants to gain a more direct biological insight of the rise and fall of particular cell states in the context of similar subsets. Applicants control the second factor of sampling depth differences by computing a normalized cell count score per patient of the form (ncells in subset/ncells in patient's major cell type)*1e6. This score provides Applicants with the number of cells expected per million.

### Mann-Whitney Tests

Applicants input the cells per million score into a two-sample Wilcoxon test in base R, which is equivalent to the Mann-Whitney rank score test. Applicants set a significance threshold of p_value <0.05. Applicants made 5 different pairwise comparisons (FGID vs FR, FGID vs PR, NOA vs FR, NOA vs PR, FR vs PR). Comparisons between FGID and pediCD groups were determined by finding maximum correspondence between the disease conditions for each subset. Due to the interest in not only finding differences between FGID and CD, but also baseline differences within CD that lead to different treatment response, Applicants are slightly underpowered in comparisons within CD, splitting the sample size from 14, to 4, 5, and 5. While Applicants do find significantly enriched subsets between subsets of CD, they are not necessarily robust to multiple testing correction. However, Applicants are confident that the split is justified. First because Applicants determined the split based on robust clinical markers (see clinical methods section). Second because Applicants do find consistent biological changes across numerous analyses. Applicants are additionally confident in the results of the Mann-Whitney tests as they correspond to the largest effect size changes as considered significant in the more lenient Fisher's exact test.

### Fisher Exact Tests

A similar compositional analysis to that done with the Mann-Whitney was performed with a Fisher's Exact test. Do the difference of the tests Applicants input for each subset the number of cells for that subset against the number of cells not of that subset within the major cell type split on rows by pairwise comparisons (NOA vs FR, NOA vs PR, FR vs PR). Applicants computed FDR correction of p_values at major cell type and entire dataset levels and found significance subsets at both levels. But, most interestingly in comparing the two tests Applicants found that the Mann-Whitney discovered as significant (pval<0.05) the portion of cell subsets with largest effect sizes. Understanding the limited patient number at these within CD comparisons and wanting to only report results most likely to be reproducible biology, Applicants determined to only follow those subsets reported as significant within both Mann-Whitney and Fisher's exact tests.

### Visualization of Compositional Analyses

Two visualizations of these tests proved particularly useful. The first a heatmap of cell per million score split by treatment condition in conjunction with the previously described correspondence dot plot was especially powerful. Those plots allowed Applicants to follow by eye directly from a significantly compositionally enriched subset in PR to its neighbors within CD and within FGID providing a complete picture of where to direct next analysis. The second also represents cell per million score, but as a scatter plot with a dot for each patient and grouped by treatment response. This allowed quick visual confirmation that results were not due simple to outlier error.

### Principal Component Analysis of Cell Frequencies and Correlation to Clinical Metadata

Cell frequencies were calculated per patient for cell subsets (i.e. end clusters) within parent cell types and cell subsets (i.e. end clusters) within all cells as CPM=((count/sum(count))*1e6. Principal component analysis (PCA) was performed on the resulting patient×CPM matrices using the R package stats::prcomp(., scale=TRUE). Variance explained per PC was calculated as std\hat{ }2/sum(std\hat{ }2). PCA loadings per patient and per cell subset were extracted from the prcomp( ) result. PCA1 and PCA2 from the total PCA×patient and from each celltype's PCA×patient matrix were correlated with clinical metadata using Spearman rank correlation as calculated by the R package stats::cor.test(., method=‘spearman’). P values were recorded from the cortesto call, and FDR was calculated using R's fdrtool::fdrtool(p.values, statistic=“pvalue”). For combined celltype PCA's, patient×CPM tables were concatenated before PCA.

### Gene Set Enrichment Analysis

Fold changes between patients responding or not responding to anti-TNF therapy from RISK and E-MTAB-7604 cohorts were calculated with Seurat (v4.0.3) (Haberman et al., 2014; Hao et al., 2021) and DESeq2 (v1.30.1) (Love et al., 2014) packages, respectively. GSEA analysis was performed using the fgsea R package (v1.16.0) (Korotkevich et al., 2021). Genes with similar fold changes were preranked in a random order. The code for this analysis can be found in the GitHub repository jo-m-lab.github.io/3p-PREDICT-Paper/4_GSEA/PREDICT_GSEA_final.html.

### Pseudotime Analysis of Expression Landscape

The micrograin structure found through hierarchical tiered clustering is vital for being able to directly compare like cells across disease conditions, and find significant changes in phenotype and composition within individual subsets. It is also vital to understand how those like subsets relate to each other within a disease condition and how the larger macrograin structure differs across conditions. This macrograin structure can be explored through the gradients of gene expression among cells of a major type. Pseudotime and RNA-velocity are both excellent tools for exploring these gradients. For both tools, the choice of genes directly determines the structure found within the dimensional reduction, and thus what genes are chosen as significantly location specific within the resulting landscape of cells. for the purposes, as Applicants knew Applicants would be exploring a single cell lineage, and exploring the relationships of cell states within that space, Applicants required for the dimensional reduction the genes common to that space. Applicants selected genes by performing differential expression between the major cell type and all other cell types within that disease. Applicants took the outer union of those genes. Then removed genes from the list found to be differentially expressed between disease conditions at the major cell type level. From these genes Applicants performed PCA to 50 principal components and then computed a UMAP reduction to 2 components. This selection process allows the dimensional reduction to find smooth gradients between cells and provided a common space for cells of multiple disease conditions to exist.

From this common expression landscape Applicants utilized Monocle3 cole-trapnell-lab.github.io/monocle3/(Cao et al., 2019) to extract a best estimate linear path through the space. Applicants calculated a diffusion pseudotime on allowing use to numerically estimate the distribution of cells within the expression landscape. To compute the significance of changes in that distribution Applicants used a permutation test of Hellinger distance between distributions. At each of 10,000 permutations Applicants shuffled the group ordering within the comparison pair. Applicants performed this test five times for comparisons between FGID vs FR, FGID vs PR, NOA vs FR, NOA vs PR and FR vs PR. The threshold was set as Bonferroni corrected p_value <0.05.

FGID and pediCD Integration Using STACAS

Integration of T cells from the FGID and pediCD datasets (n=29640 and 38031, respectively) was performed using the STACAS package (v1.1.0) (Andreatta and Carmona, 2021) Sankey plot was created using RAWGraphs 2.0 beta (https://github.com/rawgraphs) (Mauri et al., 2017).

### Differential Expression Testing

To calculate differential expression between FR and PR groups, for each subset with a least 50 cells in each condition Applicants used a Wilcoxon test thresholded to 0.05 Bonferroni corrected p-value and down sampled using the “max.cells.per.ident” argument within Seurat's ‘FindMarkers’ function to a maximum of 5000 cells. The limits on minimum and maximum number of cells were chosen mitigate issues with comparisons between disproportionate populations and computational efficiency. There does still exist 2 orders of magnitude between the minimum and maximum; however the subsets most of interest and reported in Table 2 are all of the same order of magnitude.

There are noted spillover effects within the expression tests. Applicants observe ubiquitous contamination of genes such IGHA1, IGHG1 as DEFA5, across all cell types and subsets. These genes are routinely found as enriched within more severe inflammation, beyond even this dataset. This is a real effect, but less than useful for understanding driving factors within individual cell subsets. So, Applicants focused on significant differentially expressed genes that also have a high pct.cells.expressing.in/pct.cells.expressing.out ratio. Applicants can then filter the subsets to find those with the most number of specific differentially expressed genes between the FR and PR groups.

### General Statistical Testing

Parameters such as sample size, number of replicates, number of independent experiments, measures of center, dispersion, and precision (mean+/−SEM) and statistical significances are reported in Figures and Figure Legends. A p-value less than 0.05 was considered significant. Where appropriate, a Bonferroni or FDR correction was used to account for multiple tests, as noted in the figure legends or Methods. All statistical tests corresponding to differential gene expression are described above and completed using R language for Statistical Computing.

### REFERENCES

Ai, L., Ren, Y., Zhu, M., Lu, S., Qian, Y., Chen, Z., and Xu, A. (2021). Synbindin restrains proinflammatory macrophage activation against microbiota and mucosal inflammation during colitis. Gut gutjnl-2020-321094.
Andreatta, M., and Carmona, S. J. (2021). STACAS: Sub-Type Anchor Correction for Alignment in Seurat to integrate single-cell RNA-seq data. Bioinformatics 37, 882-884.
Aschenbrenner, D., Quaranta, M., Banerjee, S., Ilott, N., Jansen, J., Steere, B., Chen, Y.-H., Ho, S., Cox, K., Arancibia-Circamo, C. V., et al. (2021). Deconvolution of monocyte responses in inflammatory bowel disease reveals an IL-1 cytokine network that regulates IL-23 in genetic and acquired IL-10 resistance. Gut 70, 1023-1036.
Atreya, R., Neurath, M. F., and Siegmund, B. (2020). Personalizing Treatment in IBD: Hype or Reality in 2020? Can Applicants Predict Response to Anti-TNF? Frontiers in Medicine 7, 517.
Banerjee, A., Herring, C. A., Chen, B., Kim, H., Simmons, A. J., Southard-Smith, A. N., Allaman, M. M., White, J. R., Macedonia, M. C., Mckinley, E. T., et al. (2020). Succinate Produced by Intestinal Microbes Promotes Specification of Tuft Cells to Suppress Ileal Inflammation. Gastroenterology 159, 2101-2115.e5.
Barker, N., van Es, J. H., Kuipers, J., Kujala, P., van den Born, M., Cozijnsen, M., Haegebarth, A., Korving, J., Begthel, H., Peters, P. J., et al. (2007). Identification of stem cells in small intestine and colon by marker gene Lgr5. Nature 449, 1003-1007.
Baumgart, D. C., and Sandborn, W. J. (2012). Crohn's disease. The Lancet 380, 1590-1605.
Betts, B. C., Veerapathran, A., Pidala, J., Yang, H., Horna, P., Walton, K., Cubitt, C. L., Gunawan, S., Lawrence, H. R., Lawrence, N. J., et al. (2017). Targeting Aurora kinase A and JAK2 prevents GVHD while maintaining Treg and antitumor CTL function. Sci Transl Med 9, eaai8269.
Beumer, J., Puschhof, J., Bauzi-Martinez, J., Martinez-Silgado, A., Elmentaite, R., James, K. R., Ross, A., Hendriks, D., Artegiani, B., Busslinger, G. A., et al. (2020). High-Resolution mRNA and Secretome Atlas of Human Enteroendocrine Cells. Cell 181, 1291-1306.e19.
Biton, M., Haber, A. L., Rogel, N., Burgin, G., Beyaz, S., Schnell, A., Ashenberg, O., Su, C.-W., Smillie, C., Shekhar, K., et al. (2018). T Helper Cell Cytokines Modulate Intestinal Stem Cell Renewal and Differentiation. Cell 175, 1307-1320.e22.
Bjorklund, A. K., Forkel, M., Picelli, S., Konya, V., Theorell, J., Friberg, D., Sandberg, R., and Mjösberg, J. (2016). The heterogeneity of human CD127+ innate lymphoid cells revealed by single-cell RNA sequencing. Nat Immunol 17, 451-460.
Black, C. J., Drossman, D. A., Talley, N. J., Ruddy, J., and Ford, A. C. (2020). Functional gastrointestinal disorders: advances in understanding and management. The Lancet 396, 1664-1674.
Bleriot, C., Chakarov, S., and Ginhoux, F. (2020). Determinants of Resident Tissue Macrophage Identity and Function. Immunity 52, 957-970.
Brulois, K., Rajaraman, A., Szade, A., Nordling, S., Bogoslowski, A., Dermadi, D., Rahman, M., Kiefel, H., O'Hara, E., Koning, J. J., et al. (2020). A molecular map of murine lymph node blood vascular endothelium at single cell resolution. Nat Commun 11, 3798.
Buechler, M. B., Pradhan, R. N., Krishnamurty, A. T., Cox, C., Calviello, A. K., Wang, A. W., Yang, Y. A., Tam, L., Caothien, R., Roose-Girma, M., et al. (2021). Cross-tissue organization of the fibroblast lineage. Nature 1-5.
Buisine, M. P., Desreumaux, P., Leteurtre, E., Copin, M. C., Colombel, J. F., Porchet, N., and Aubert, J. P. (2001). Mucin gene expression in intestinal epithelial cells in Crohn's disease. Gut 49, 544-551.
Cao, J., Spielmann, M., Qiu, X., Huang, X., Ibrahim, D. M., Hill, A. J., Zhang, F., Mundlos, S., Christiansen, L., Steemers, F. J., et al. (2019). The single-cell transcriptional landscape of mammalian organogenesis. Nature 566, 496-502.
Cappello, M., and Morreale, G. C. (2016). The Role of Laboratory Tests in Crohn's Disease. Clin Med Insights Gastroenterol 9, 51-62.
Castro-Dopico, T., Fleming, A., Dennison, T. W., Ferdinand, J. R., Harcourt, K., Stewart, B. J., Cader, Z., Tuong, Z. K., Jing, C., Lok, L. S. C., et al. (2020). GM-CSF Calibrates Macrophage Defense and Wound Healing Programs during Intestinal Infection and Inflammation. Cell Reports 32, 107857.
Catalan-Serra, I., Sandvik, A. K., Bruland, T., and Andreu-Ballester, J. C. (2017). Gammadelta T Cells in Crohn's Disease: A New Player in the Disease Pathogenesis? Journal of Crohn's and Colitis 11, 1135-1145.
Chang, J. T. (2020). Pathophysiology of Inflammatory Bowel Diseases. New England Journal of Medicine 383, 2652-2664.
Cherrier, D. E., Serafini, N., and Di Santo, J. P. (2018). Innate Lymphoid Cell Development: A T Cell Perspective. Immunity 48, 1091-1103.
Cima, I., Corazza, N., Dick, B., Fuhrer, A., Herren, S., Jakob, S., Ayuni, E., Mueller, C., and Brunner, T. (2004). Intestinal Epithelial Cells Synthesize Glucocorticoids and Regulate T Cell Activation. J Exp Med 200, 1635-1646.
Cohen, L. J., Cho, J. H., Gevers, D., and Chu, H. (2019). Genetic Factors and the Intestinal Microbiome Guide Development of Microbe-Based Therapies for Inflammatory Bowel Diseases. Gastroenterology 156, 2174-2189.
Corridoni, D., Chapman, T., Antanaviciute, A., Satsangi, J., and Simmons, A. (2020a). Inflammatory Bowel Disease Through the Lens of Single-cell RNA-seq Technologies. Inflammatory Bowel Diseases 26, 1658-1668.
Corridoni, D., Antanaviciute, A., Gupta, T., Fawkner-Corbett, D., Aulicino, A., Jagielowicz, M., Parikh, K., Repapi, E., Taylor, S., Ishikawa, D., et al. (2020b). Single-cell atlas of colonic CD8+ T cells in ulcerative colitis. Nat Med 26, 1480-1490.
Cyster, J. G., and Allen, C. D. C. (2019). B Cell Responses: Cell Interaction Dynamics and Decisions. Cell 177, 524-540.
Das, A., Heesters, B. A., Bialas, A., O'Flynn, J., Rifkin, I. R., Ochando, J., Mittereder, N., Carlesso, G., Herbst, R., and Carroll, M. C. (2017). Follicular Dendritic Cell Activation by TLR Ligands Promotes Autoreactive B Cell Responses. Immunity 46, 106-119.
Davidson, S., Coles, M., Thomas, T., Kollias, G., Ludewig, B., Turley, S., Brenner, M., and Buckley, C. D. (2021). Fibroblasts as immune regulators in infection, inflammation and cancer. Nature Reviews Immunology 1-14.
D'Haens, G. R., and Deventer, S. van (2021). 25 years of anti-TNF treatment for inflammatory bowel disease: lessons from the past and a look to the future. Gut 70, 1396-1405.
Digby-Bell, J. L., Atreya, R., Monteleone, G., and Powell, N. (2020). Interrogating host immunity to predict treatment response in inflammatory bowel disease. Nat Rev Gastroenterol Hepatol 17, 9-20.
Dominguez-Sola, D., Victora, G. D., Ying, C. Y., Phan, R. T., Saito, M., Nussenzweig, M. C., and Dalla-Favera, R. (2012). The proto-oncogene MYC is required for selection in the germinal center and cyclic reentry. Nat Immunol 13, 1083-1091.
Dovrolis, N., Michalopoulos, G., Theodoropoulos, G. E., Arvanitidis, K., Kolios, G., Sechi, L. A., Eliopoulos, A. G., and Gazouli, M. (2020). The Interplay between Mucosal Microbiota Composition and Host Gene-Expression is Linked with Infliximab Response in Inflammatory Bowel Diseases. Microorganisms 8, 438.
Drokhlyansky, E., Smillie, C. S., Wittenberghe, N. V., Ericsson, M., Griffin, G. K., Dionne, D., Cuoco, M. S., Goder-Reiser, M. N., Sharova, T., Aguirre, A. J., et al. (2019). The enteric nervous system of the human and mouse colon at a single-cell resolution. BioRxiv 746743.
Dutertre, C.-A., Becht, E., Irac, S. E., Khalilnezhad, A., Narang, V., Khalilnezhad, S., Ng, P. Y., van den Hoogen, L. L., Leong, J. Y., Lee, B., et al. (2019). Single-Cell Analysis of Human Mononuclear Phagocytes Reveals Subset-Defining Markers and Identifies Circulating Inflammatory Dendritic Cells. Immunity 51, 573-589.e8.
Dwyer, D. F., Ordovas-Montanes, J., Allon, S. J., Buchheit, K. M., Vukovic, M., Derakhshan, T., Feng, C., Lai, J., Hughes, T. K., Nyquist, S. K., et al. (2021). Human airway mast cells proliferate and acquire distinct inflammation-driven phenotypes during type 2 inflammation. Sci Immunol 6, eabb7221.
Elmentaite, R., Ross, A. D. B., Roberts, K., James, K. R., Ortmann, D., Gomes, T., Nayak, K., Tuck, L., Pritchard, S., Bayraktar, O. A., et al. (2020). Single-Cell Sequencing of Developing Human Gut Reveals Transcriptional Links to Childhood Crohn's Disease. Developmental Cell 55, 771-783.e5.
van der Flier, L. G., and Clevers, H. (2009). Stem Cells, Self-Renewal, and Differentiation in the Intestinal Epithelium. Annu. Rev. Physiol. 71, 241-260.
Franzosa, E. A., Sirota-Madi, A., Avila-Pacheco, J., Fornelos, N., Haiser, H. J., Reinker, S., Vatanen, T., Hall, A. B., Mallick, H., McIver, L. J., et al. (2019). Gut microbiome structure and metabolic activity in inflammatory bowel disease. Nat Microbiol 4, 293-305.
French, A. R., Sjolin, H., Kim, S., Koka, R., Yang, L., Young, D. A., Cerboni, C., Tomasello, E., Ma, A., Vivier, E., et al. (2006). DAP12 Signaling Directly Augments Proproliferative Cytokine Stimulation of NK Cells during Viral Infections. The Journal of Immunology 177, 4981-4990.
Friedrich, M., Pohin, M., and Powrie, F. (2019). Cytokine Networks in the Pathophysiology of Inflammatory Bowel Disease. Immunity 50, 992-1006.
Furey, T. S., Sethupathy, P., and Sheikh, S. Z. (2019). Redefining the IBDs using genome-scale molecular phenotyping. Nat Rev Gastroenterol Hepatol 16, 296-311.
Gomariz, A., Helbling, P. M., Isringhausen, S., Suessbier, U., Becker, A., Boss, A., Nagasawa, T., Paul, G., Goksel, O., Szekely, G., et al. (2018). Quantitative spatial analysis of haematopoiesis-regulating stromal cells in the bone marrow microenvironment by 3D microscopy. Nat Commun 9, 2532.
Graham, D. B., and Xavier, R. J. (2020). Pathway paradigms revealed from the genetics of inflammatory bowel disease. Nature 578, 527-539.
Guilliams, M., Mildner, A., and Yona, S. (2018). Developmental and Functional Heterogeneity of Monocytes. Immunity 49, 595-613.
Haberman, Y., Tickle, T. L., Dexheimer, P. J., Kim, M.-O., Tang, D., Karns, R., Baldassano, R. N., Noe, J. D., Rosh, J., Markowitz, J., et al. (2014). Pediatric Crohn disease patients exhibit specific ileal transcriptome and microbiome signature. J Clin Invest 124, 3617-3633.
Hafemeister, C., and Satija, R. (2019). Normalization and variance stabilization of single-cell RNA-seq data using regularized negative binomial regression. Genome Biology 20, 296.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell.
Heesters, B. A., Chatterjee, P., Kim, Y.-A., Gonzalez, S. F., Kuligowski, M. P., Kirchhausen, T., and Carroll, M. C. (2013). Endocytosis and Recycling of Immune Complexes by Follicular Dendritic Cells Enhances B Cell Antigen Binding and Activation. Immunity 38, 1164-1175.
Hie, B., Bryson, B., and Berger, B. (2019). Efficient integration of heterogeneous single-cell transcriptomes using Scanorama. Nat Biotechnol 37, 685-691.
Hie, B., Peters, J., Nyquist, S. K., Shalek, A. K., Berger, B., and Bryson, B. D. (2020). Computational Methods for Single-Cell RNA Sequencing. Annu. Rev. Biomed. Data Sci. 3, 339-364.
Huang, B., Chen, Z., Geng, L., Wang, J., Liang, H., Cao, Y., Chen, H., Huang, W., Su, M., Wang, H., et al. (2019). Mucosal Profiling of Pediatric-Onset Colitis and IBD Reveals Common Pathogenics and Therapeutic Pathways. Cell 179, 1160-1176.e24.
Hyams, J. S., Ferry, G. D., Mandel, F. S., Gryboski, J. D., Kibort, P. M., Kirschner, B. S., Griffiths, A. M., Katz, A. J., Grand, R. J., Boyle, J. T., et al. (1991). Development and Validation of a Pediatric Crohn's Disease Activity Index. Journal of Pediatric Gastroenterology and Nutrition 12, 439.
Hyams, J. S., Di Lorenzo, C., Saps, M., Shulman, R. J., Staiano, A., and van Tilburg, M. (2016). Childhood Functional Gastrointestinal Disorders: Child/Adolescent. Gastroenterology 150, 1456-1468.e2.
Jain, U., Heul, A. M. V., Xiong, S., Gregory, M. H., Demers, E. G., Kern, J. T., Lai, C.-W., Muegge, B. D., Barisas, D. A. G., Leal-Ekman, J. S., et al. (2021). Debaryomyces is enriched in Crohn's disease intestinal tissue and impairs healing in mice. Science 371, 1154-1159.
James, K. R., Gomes, T., Elmentaite, R., Kumar, N., Gulliver, E. L., King, H. W., Stares, M. D., Bareham, B. R., Ferdinand, J. R., Petrova, V. N., et al. (2020). Distinct microbial and immune niches of the human colon. Nat Immunol 21, 343-353.
Kaczorowski, K. J., Shekhar, K., Nkulikiyimfura, D., Dekker, C. L., Maecker, H., Davis, M. M., Chakraborty, A. K., and Brodin, P. (2017). Continuous immunotypes describe human immune variation and predict diverse responses. PNAS 114, E6097-E6106.
Kinchen, J., Chen, H. H., Parikh, K., Antanaviciute, A., Jagielowicz, M., Fawkner-Corbett, D., Ashley, N., Cubitt, L., Mellado-Gomez, E., Attar, M., et al. (2018). Structural Remodeling of the Human Colonic Mesenchyme in Inflammatory Bowel Disease. Cell 175, 372-386.e17.
Kobayashi, T., Siegmund, B., Le Berre, C., Wei, S. C., Ferrante, M., Shen, B., Bernstein, C. N., Danese, S., Peyrin-Biroulet, L., and Hibi, T. (2020). Ulcerative colitis. Nat Rev Dis Primers 6, 1-20.
Korotkevich, G., Sukhov, V., Budin, N., Shpak, B., Artyomov, M. N., and Sergushichev, A. (2021). Fast gene set enrichment analysis.
Korsunsky, I., Millard, N., Fan, J., Slowikowski, K., Zhang, F., Wei, K., Baglaenko, Y., Brenner, M., Loh, P., and Raychaudhuri, S. (2019). Fast, sensitive and accurate integration of single-cell data with Harmony. Nat Methods 16, 1289-1296.
Kugathasan, S., Denson, L. A., Walters, T. D., Kim, M.-O., Marigorta, U. M., Schirmer, M., Mondal, K., Liu, C., Griffiths, A., Noe, J. D., et al. (2017). Prediction of complicated disease course for children newly diagnosed with Crohn's disease: a multicentre inception cohort study. Lancet 389, 1710-1718.
La Manno, G., Siletti, K., Furlan, A., Gyllborg, D., Vinsland, E., Mossi Albiach, A., Mattsson Langseth, C., Khven, I., Lederer, A. R., Dratva, L. M., et al. (2021). Molecular architecture of the developing mouse brain. Nature 596, 92-96.
Lampen, A., Meyer, S., Arnhold, T., and Nau, H. (2000). Metabolism of vitamin A and its active metabolite all-trans-retinoic acid in small intestinal enterocytes. J Pharmacol Exp Ther 295, 979-985.
Lanier, L. L. (2001). On guard-activating NK cell receptors. Nat Immunol 2, 23-27.
Lanier, L. L., Corliss, B., Wu, J., and Phillips, J. H. (1998). Association of DAP12 with Activating CD94/NKG2C NK Cell Receptors. Immunity 8, 693-701.
Leach, S. T., Yang, Z., Messina, I., Song, C., Geczy, C. L., Cunningham, A. M., and Day, A. S. (2007). Serum and mucosal S100 proteins, calprotectin (S100A8/S100A9) and S100A12, are elevated at diagnosis in children with inflammatory bowel disease. Scandinavian Journal of Gastroenterology 42, 1321-1331.
Leeb, S. N., Vogl, D., Gunckel, M., Kiessling, S., Falk, W., Göke, M., Schölmerich, J., Gelbmann, C. M., and Rogler, G. (2003). Reduced migration of fibroblasts in inflammatory bowel disease: role of inflammatory mediators and focal adhesion kinase. Gastroenterology 125, 1341-1354.
Leonard, N., Hourihane, D. O., and Whelan, A. (1995). Neuroproliferation in the mucosa is a feature of coeliac disease and Crohn's disease. Gut 37, 763-765.
Levine, A., Griffiths, A., Markowitz, J., Wilson, D. C., Turner, D., Russell, R. K., Fell, J., Ruemmele, F. M., Walters, T., Sherlock, M., et al. (2011). Pediatric modification of the Montreal classification for inflammatory bowel disease: the Paris classification. Inflamm Bowel Dis 17, 1314-1321.
Lilja, I., Gustafson-Svärd, C., Franzen, L., and Sjödahl, R. (2000). Tumor Necrosis Factor-Alpha in Ileal Mast Cells in Patients with Crohn's Disease. DIG 61, 68-76.
Limon, J. J., Tang, J., Li, D., Wolf, A. J., Michelsen, K. S., Funari, V., Gargus, M., Nguyen, C., Sharma, P., Maymi, V. I., et al. (2019). Malassezia Is Associated with Crohn's Disease and Exacerbates Colitis in Mouse Models. Cell Host & Microbe 25, 377-388.e6.
Lindemans, C. A., Calafiore, M., Mertelsmann, A. M., O'Connor, M. H., Dudakov, J. A., Jenq, R. R., Velardi, E., Young, L. F., Smith, O. M., Lawrence, G., et al. (2015). Interleukin-22 promotes intestinal-stem-cell-mediated epithelial regeneration. Nature 528, 560-564.
Love, M. I., Huber, W., and Anders, S. (2014). Moderated estimation of fold change and dispersion for RNA-seq data with DESeq2. Genome Biology 15, 550.
Lucas, C., Wong, P., Klein, J., Castro, T. B. R., Silva, J., Sundaram, M., Ellingson, M. K., Mao, T., Oh, J. E., Israelow, B., et al. (2020). Longitudinal analyses reveal immunological misfiring in severe COVID-19. Nature 584, 463-469.
Mabbott, N. A., Donaldson, D. S., Ohno, H., Williams, I. R., and Mahajan, A. (2013). Microfold (M) cells: important immunosurveillance posts in the intestinal epithelium. Mucosal Immunol 6, 666-677.
Magro, F., Vieira-Coelho, M. A., Fraga, S., Serrao, M. P., Veloso, F. T., Ribeiro, T., and Soares-da-Silva, P. (2002). Impaired Synthesis or Cellular Storage of Norepinephrine, Dopamine, and 5-Hydroxytryptamine in Human Inflammatory Bowel Disease. Dig Dis Sci 47, 216-224.
Mirtensson, J., Jain, A., and Meister, A. (1990). Glutathione is required for intestinal function. Proc Natl Acad Sci USA 87, 1715-1719.
Martin, J. C., Chang, C., Boschetti, G., Ungaro, R., Giri, M., Grout, J. A., Gettler, K., Chuang, L., Nayar, S., Greenstein, A. J., et al. (2019). Single-Cell Analysis of Crohn's Disease Lesions Identifies a Pathogenic Cellular Module Associated with Resistance to Anti-TNF Therapy. Cell 178, 1493-1508.e20.
Martinez-Augustin, O., and de Medina, F. S. (2008). Intestinal bile acid physiology and pathophysiology. World J Gastroenterol 14, 5630-5640.
Mathew, D., Giles, J. R., Baxter, A. E., Oldridge, D. A., Greenplate, A. R., Wu, J. E., Alanio, C., Kuri-Cervantes, L., Pampena, M. B., D'Andrea, K., et al. (2020). Deep immune profiling of COVID-19 patients reveals distinct immunotypes with therapeutic implications. Science 369.
Mauri, M., Elli, T., Caviglia, G., Uboldi, G., and Azzi, M. (2017). RAWGraphs: A Visualisation Platform to Create Open Outputs. In Proceedings of the 12th Biannual Conference on Italian SIGCHI Chapter, (New York, NY, USA: Association for Computing Machinery), pp. 1-5.
McOmber, M. A., and Shulman, R. J. (2008). Pediatric Functional Gastrointestinal Disorders. Nutrition in Clinical Practice 23, 268-274.
Mehta, P., Porter, J. C., Manson, J. J., Isaacs, J. D., Openshaw, P. J. M., McInnes, I. B., Summers, C., and Chambers, R. C. (2020). Therapeutic blockade of granulocyte macrophage colony-stimulating factor in COVID-19-associated hyperinflammation: challenges and opportunities. The Lancet Respiratory Medicine 8, 822-830.
Meijer, C. J. L. M., Bosman, F. T., and Lindeman, J. (1979). Evidence for Predominant Involvement of the B-Cell System in the Inflammatory Process in Crohn's Disease. Scandinavian Journal of Gastroenterology 14, 21-32.
Mitsialis, V., Wall, S., Liu, P., Ordovas-Montanes, J., Parmet, T., Vukovic, M., Spencer, D., Field, M., McCourt, C., Toothaker, J., et al. (2020). Single-Cell Analyses of Colon and Blood Reveal Distinct Immune Cell Signatures of Ulcerative Colitis and Crohn's Disease. Gastroenterology 159, 591-608.e10.
Miura, A., Sootome, H., Fujita, N., Suzuki, T., Fukushima, H., Mizuarai, S., Masuko, N., Ito, K., Hashimoto, A., Uto, Y., et al. (2021). TAS-119, a novel selective Aurora A and TRK inhibitor, exhibits antitumor efficacy in preclinical models with deregulated activation of the Myc, β-Catenin, and TRK pathways. Invest New Drugs 39, 724-735.
von Moltke, J., Ji, M., Liang, H.-E., and Locksley, R. M. (2016). Tuft-cell-derived IL-25 regulates an intestinal ILC2-epithelial response circuit. Nature 529, 221-225.
Moor, A. E., Harnik, Y., Ben-Moshe, S., Massasa, E. E., Rozenberg, M., Eilam, R., Bahar Halpern, K., and Itzkovitz, S. (2018). Spatial Reconstruction of Single Enterocytes Uncovers Broad Zonation along the Intestinal Villus Axis. Cell 175, 1156-1167.e15.
Müller, S., Lory, J., Corazza, N., Griffiths, G. M., Z'graggen, K., Mazzucchelli, L., Kappeler, A., and Mueller, C. (1998). Activated CD4+ and CD8+ cytotoxic cells are present in increased numbers in the intestinal mucosa from patients with active inflammatory bowel disease. Am J Pathol 152, 261-268.
Muro, M., and Mrowiec, A. (2015). Interleukin (IL)-1 Gene Cluster in Inflammatory Bowel Disease: Is IL-1RA Implicated in the Disease Onset and Outcome? Dig Dis Sci 60, 1126-1128.
Neurath, M. F. (2019). Targeting immune cell circuits and trafficking in inflammatory bowel disease. Nat Immunol 20, 970-979.
Ordis, I., Mould, D. R., Feagan, B. G., and Sandborn, W. J. (2012). Anti-TNF monoclonal antibodies in inflammatory bowel disease: pharmacokinetics-based dosing paradigms. Clin Pharmacol Ther 91, 635-646.
Ordovas-Montanes, J., Dwyer, D. F., Nyquist, S. K., Buchheit, K. M., Vukovic, M., Deb, C., Wadsworth, M. H., Hughes, T. K., Kazer, S. W., Yoshimoto, E., et al. (2018). Allergic inflammatory memory in human respiratory epithelial progenitor cells. Nature 560, 649-654.
Parikh, K., Antanaviciute, A., Fawkner-Corbett, D., Jagielowicz, M., Aulicino, A., Lagerholm, C., Davis, S., Kinchen, J., Chen, H. H., Alham, N. K., et al. (2019). Colonic epithelial cell diversity in health and inflammatory bowel disease. Nature 567, 49-55.
Pedregosa, F., Varoquaux, G., Gramfort, A., Michel, V., Thirion, B., Grisel, O., Blondel, M., Prettenhofer, P., Weiss, R., Dubourg, V., et al. (2011). Scikit-learn: Machine Learning in Python. Journal of Machine Learning Research 12, 2825-2830.
Peng, Y.-R., Shekhar, K., Yan, W., Herrmann, D., Sappington, A., Bryman, G. S., van Zyl, T., Do, M.Tri.H., Regev, A., and Sanes, J. R. (2019). Molecular Classification and Comparative Taxonomics of Foveal and Peripheral Cells in Primate Retina. Cell 176, 1222-1237.e22.
Pereira, J. P., Kelly, L. M., Xu, Y., and Cyster, J. G. (2009). EBI2 mediates B cell segregation between the outer and centre follicle. Nature 460, 1122-1126.
Persson, E. K., Uronen-Hansson, H., Semmrich, M., Rivollier, A., Hagerbrand, K., Marsal, J., Gudjonsson, S., Hikansson, U., Reizis, B., Kotarsky, K., et al. (2013). IRF4 Transcription-Factor-Dependent CD103+CD11b+ Dendritic Cells Drive Mucosal T Helper 17 Cell Differentiation. Immunity 38, 958-969.
Pliner, H. A., Shendure, J., and Trapnell, C. (2019). Supervised classification enables rapid annotation of cell atlases. Nat Methods 16, 983-986.
Rajca, S., Grondin, V., Louis, E., Vernier-Massouille, G., Grimaud, J.-C., Bouhnik, Y., Laharie, D., Dupas, J.-L., Pillant, H., Picon, L., et al. (2014). Alterations in the Intestinal Microbiome (Dysbiosis) as a Predictor of Relapse After Infliximab Withdrawal in Crohn's Disease. Inflammatory Bowel Diseases 20, 978-986.
Ramanujam, M., Steffgen, J., Visvanathan, S., Mohan, C., Fine, J. S., and Putterman, C. (2020). Phoenix from the flames: Rediscovering the role of the CD40-CD40L pathway in systemic lupus erythematosus and lupus nephritis. Autoimmunity Reviews 19, 102668.
Renoux, V. M., Zriwil, A., Peitzsch, C., Michaëlsson, J., Friberg, D., Soneji, S., and Sitnicka, E. (2015). Identification of a Human Natural Killer Cell Lineage-Restricted Progenitor in Fetal and Adult Tissues. Immunity 43, 394-407.
Robinette, M. L., and Colonna, M. (2016). Immune modules shared by innate lymphoid cells and T cells. J Allergy Clin Immunol 138, 1243-1251.
Roda, G., Chien Ng, S., Kotze, P. G., Argollo, M., Panaccione, R., Spinelli, A., Kaser, A., Peyrin-Biroulet, L., and Danese, S. (2020). Crohn's disease. Nat Rev Dis Primers 6, 1-19.
Roncarolo, M. G., Gregori, S., Bacchetta, R., Battaglia, M., and Gagliani, N. (2018). The Biology of T Regulatory Type 1 Cells and Their Therapeutic Application in Immune-Mediated Diseases. Immunity 49, 1004-1019.
Rousseeuw, P. J. (1987). Silhouettes: A graphical aid to the interpretation and validation of cluster analysis. Journal of Computational and Applied Mathematics 20, 53-65.
Ruemmele, F. M., Veres, G., Kolho, K. L., Griffiths, A., Levine, A., Escher, J. C., Amil Dias, J., Barabino, A., Braegger, C. P., Bronsky, J., et al. (2014). Consensus guidelines of ECCO/ESPGHAN on the medical management of pediatric Crohn's disease. J Crohns Colitis 8, 1179-1207.
Sallusto, F., Lenig, D., Forster, R., Lipp, M., and Lanzavecchia, A. (1999). Two subsets of memory T lymphocytes with distinct homing potentials and effector functions. Nature 401, 708-712.
Sandborn, W. J. (2014). Crohn's Disease Evaluation and Treatment: Clinical Decision Tool. Gastroenterology 147, 702-705.
Santucci, N. R., Saps, M., and van Tilburg, M. A. (2020). New advances in the treatment of paediatric functional abdominal pain disorders. The Lancet Gastroenterology & Hepatology 5, 316-328.
Schulte-Schrepping, J., Reusch, N., Paclik, D., Baßler, K., Schlickeiser, S., Zhang, B., Kramer, B., Krammer, T., Brumhard, S., Bonaguro, L., et al. (2020). Severe COVID-19 Is Marked by a Dysregulated Myeloid Cell Compartment. Cell 182, 1419-1440.e23.
Selin, K. A., Hedin, C. R. H., and Villablanca, E. J. (2021). Immunological networks defining the heterogeneity of inflammatory bowel diseases. Journal of Crohn's and Colitis.
Shekhar, K., Lapan, S. W., Whitney, I. E., Tran, N. M., Macosko, E. Z., Kowalczyk, M., Adiconis, X., Levin, J. Z., Nemesh, J., Goldman, M., et al. (2016). COMPREHENSIVE CLASSIFICATION OF RETINAL BIPOLAR NEURONS BY SINGLE-CELL TRANSCRIPTOMICS. Cell 166, 1308-1323.e30.
Sido, B., Hack, V., Hochlehnert, A., Lipps, H., Herfarth, C., and Droge, W. (1998). Impairment of intestinal glutathione synthesis in patients with inflammatory bowel disease. Gut 42, 485-492.
Sieber, G., Herrmann, F., Zeitz, M., Teichmann, H., and Rühl, H. (1984). Abnormalities of B-cell activation and immunoregulation in patients with Crohn's disease. Gut 25, 1255-1261.
Silverberg, M. S., Satsangi, J., Ahmad, T., Arnott, I. D. R., Bernstein, C. N., Brant, S. R., Caprilli, R., Colombel, J.-F., Gasche, C., Geboes, K., et al. (2005). Toward an integrated clinical, molecular and serological classification of inflammatory bowel disease: report of a Working Party of the 2005 Montreal World Congress of Gastroenterology. Can J Gastroenterol 19 SupplA, 5A-36A.
Simpson, E. H. (1949). Measurement of Diversity. Nature 163, 688-688.
Smillie, C. S., Biton, M., Ordovas-Montanes, J., Sullivan, K. M., Burgin, G., Graham, D. B., Herbst, R. H., Rogel, N., Slyper, M., Waldman, J., et al. (2019). Intra- and Inter-cellular Rewiring of the Human Colon during Ulcerative Colitis. Cell 178, 714-730.e22.
Sootome, H., Miura, A., Masuko, N., Suzuki, T., Uto, Y., and Hirai, H. (2020). Aurora A Inhibitor TAS-119 Enhances Antitumor Efficacy of Taxanes In Vitro and In Vivo: Preclinical Studies as Guidance for Clinical Development and Trial Design. Mol Cancer Ther 19, 1981-1991.
Souza, H. S., Elia, C. C. S., Spencer, J., and MacDonald, T. T. (1999). Expression of lymphocyte-endothelial receptor-ligand pairs, α4β7/MAdCAM-1 and OX40/OX40 ligand in the colon and jejunum of patients with inflammatory bowel disease. Gut 45, 856-863.
Stappenbeck, T. S., and McGovern, D. P. B. (2017). Paneth Cell Alterations in the Development and Phenotype of Crohn's Disease. Gastroenterology 152, 322-326.
Stevens, T. W., Matheeuwsen, M., Lonnkvist, M. H., Parker, C. E., Wildenberg, M. E., Gecse, K. B., and D'Haens, G. R. (2018). Systematic review: predictive biomarkers of therapeutic response in inflammatory bowel disease-personalised medicine in its infancy. Aliment Pharmacol Ther 48, 1213-1231.
Stuart, T., Butler, A., Hoffman, P., Hafemeister, C., Papalexi, E., Mauck, W. M., Hao, Y., Stoeckius, M., Smibert, P., and Satija, R. (2019). Comprehensive Integration of Single-Cell Data. Cell 177, 1888-1902.e21.
Su, Y., Chen, D., Yuan, D., Lausted, C., Choi, J., Dai, C. L., Voillet, V., Duvvuri, V. R., Scherler, K., Troisch, P., et al. (2020). Multi-Omics Resolves a Sharp Disease-State Shift between Mild and Moderate COVID-19. Cell 183, 1479-1495.e20.
Sullivan, Z. A., Khoury-Hanold, W., Lim, J., Smillie, C., Biton, M., Reis, B. S., Zwick, R. K., Pope, S. D., Israni-Winger, K., Parsa, R., et al. (2021). 76 T cells regulate the intestinal response to nutrient sensing. Science 371.
Sykora, J., Pomahacovi, R., Kreslovi, M., Cvalinovi, D., Stych, P., and Schwarz, J. (2018). Current global trends in the incidence of pediatric-onset inflammatory bowel disease. World J Gastroenterol 24, 2741-2763.
Takayama, T., Kamada, N., Chinen, H., Okamoto, S., Kitazume, M. T., Chang, J., Matuzaki, Y., Suzuki, S., Sugita, A., Koganei, K., et al. (2010). Imbalance of NKp44+ NKp46− and NKp44−NKp46+ Natural Killer Cells in the Intestinal Mucosa of Patients With Crohn's Disease. Gastroenterology 139, 882-892.e3.
Tasic, B., Yao, Z., Graybuck, L. T., Smith, K. A., Nguyen, T. N., Bertagnolli, D., Goldy, J., Garren, E., Economo, M. N., Viswanathan, S., et al. (2018). Shared and distinct transcriptomic cell types across neocortical areas. Nature 563, 72-78.
Thiriot, A., Perdomo, C., Cheng, G., Novitzky-Basso, I., McArdle, S., Kishimoto, J. K., Barreiro, O., Mazo, I., Triboulet, R., Ley, K., et al. (2017). Differential DARC/ACKR1 expression distinguishes venular from non-venular endothelial cells in murine tissues. BMC Biology 15, 45.
Travaglini, K. J., Nabhan, A. N., Penland, L., Sinha, R., Gillich, A., Sit, R. V., Chang, S., Conley, S. D., Mori, Y., Seita, J., et al. (2020). A molecular cell atlas of the human lung from single-cell RNA sequencing. Nature 587, 619-625.
Turner, D., Griffiths, A. M., Walters, T. D., Seah, T., Markowitz, J., Pfefferkorn, M., Keljo, D., Waxman, J., Otley, A., LeLeiko, N. S., et al. (2012). Mathematical weighting of the pediatric Crohn's disease activity index (PCDAI) and comparison with its other short versions. Inflamm Bowel Dis 18, 55-62.
Turner, D., Levine, A., Walters, T. D., Focht, G., Otley, A., L6pez, V. N., Koletzko, S., Baldassano, R., Mack, D., Hyams, J., et al. (2017). Which PCDAI Version Best Reflects Intestinal Inflammation in Pediatric Crohn Disease? Journal of Pediatric Gastroenterology and Nutrition 64, 254-260.
Verstockt, B., Verstockt, S., Dehairs, J., Ballet, V., Blevi, H., Wollants, W.-J., Breynaert, C., Van Assche, G., Vermeire, S., and Ferrante, M. (2019). Low TREM1 expression in whole blood predicts anti-TNF response in inflammatory bowel disease. EBioMedicine 40, 733-742.
Victora, G. D., Schwickert, T. A., Fooksman, D. R., Kamphorst, A. O., Meyer-Hermann, M., Dustin, M. L., and Nussenzweig, M. C. (2010). Germinal Center Dynamics Revealed by Multiphoton Microscopy with a Photoactivatable Fluorescent Reporter. Cell 143, 592-605.
Wen, J., and Rawls, J. F. (2020). Feeling the Burn: Intestinal Epithelial Cells Modify Their Lipid Metabolism in Response to Bacterial Fermentation Products. Cell Host & Microbe 27, 314-316.
Whitsett, J. A., Kalin, T. V., Xu, Y., and Kalinichenko, V. V. (2019). Building and Regenerating the Lung Cell by Cell. Physiol Rev 99, 513-554.
Yarur, A. J., Jain, A., Sussman, D. A., Barkin, J. S., Quintero, M. A., Princen, F., Kirkland, R., Deshpande, A. R., Singh, S., and Abreu, M. T. (2016). The association of tissue anti-TNF drug levels with serological and endoscopic disease activity in inflammatory bowel disease: the ATLAS study. Gut 65, 249-255.
Ye, Y., Manne, S., Treem, W. R., and Bennett, D. (2020). Prevalence of Inflammatory Bowel Disease in Pediatric and Adult Populations: Recent Estimates From Large National Databases in the United States, 2007-2016. Inflamm Bowel Dis 26, 619-625.
Yilmaz, B., Juillerat, P., Oyis, O., Ramon, C., Bravo, F. D., Franc, Y., Fournier, N., Michetti, P., Mueller, C., Geuking, M., et al. (2019). Microbial network disturbances in relapsing refractory Crohn's disease. Nat Med 25, 323-336.
Zeisel, A., Hochgerner, H., Lonnerberg, P., Johnsson, A., Memic, F., van der Zwan, J., Haring, M., Braun, E., Borm, L. E., La Manno, G., et al. (2018). Molecular Architecture of the Mouse Nervous System. Cell 174, 999-1014.e22.
Ziegler, C. G. K., Allon, S. J., Nyquist, S. K., Mbano, I. M., Miao, V. N., Tzouanas, C. N., Cao, Y., Yousif, A. S., Bals, J., Hauser, B. M., et al. (2020). SARS-CoV-2 Receptor ACE2 Is an Interferon-Stimulated Gene in Human Airway Epithelial Cells and Is Detected in Specific Cell Subsets across Tissues. Cell 181, 1016-1035.e19.
Ziegler, C. G. K., Miao, V. N., Owings, A. H., Navia, A. W., Tang, Y., Bromley, J. D., Lotfy, P., Sloan, M., Laird, H., Williams, H. B., et al. (2021). Impaired local intrinsic immunity to SARS-CoV-2 infection in severe COVID-19. Cell.

### Tables

TABLE 1A
Markers for all cell subsets of Tier 1 cell types in CD atlas (ordered by adj p value for each subset)

| | | avg_{—} | | | avg_{—} | | | avg_{—} |
|---|---|---|---|---|---|---|---|---|
| cluster | gene | logFC | cluster | gene | logFC | cluster | gene | logFC |
| CD.B.FCRL4.OSTN-AS1 | FCRL4 | 1.23 | CD.Endth/Mural.HIGD1B.NDUFA4L2 | FILIP1 | 0.80 | CD.ILC.AREG.AHR | ALDOC | 2.14 |
| CD.B.FCRL4.OSTN-AS1 | OSTN-AS1 | 0.84 | CD.Endth/Mural.HIGD1B.NDUFA4L2 | FAM110D | 0.80 | CD.ILC.IL22.KIT | KLRC1 | 2.26 |
| CD.B.FCRL4.OSTN-AS1 | BANK1 | 1.22 | CD.Endth/Mural.HIGD1B.NDUFA4L2 | KANK3 | 0.79 | CD.ILC.IL22.KIT | KRT81 | 2.03 |
| CD.B.FCRL4.OSTN-AS1 | SPIB | 1.09 | CD.Endth/Mural.HIGD1B.NDUFA4L2 | TIE1 | 0.79 | CD.ILC.IL22.KIT | KRT86 | 1.88 |
| CD.B.FCRL4.OSTN-AS1 | TLR10 | 0.89 | CD.Endth/Mural.HIGD1B.NDUFA4L2 | GJA4 | 0.78 | CD.ILC.IL22.KIT | NCR2 | 0.78 |
| CD.B.HCK.DUSP4 | HLA-DQA2 | 1.88 | CD.Endth/Mural.HIGD1B.NDUFA4L2 | RCAN2 | 0.76 | CD.ILC.KRT81.IL22 | ALDOC | 3.00 |
| CD.B.HCK.DUSP4 | CD19 | 1.51 | CD.Endth/Mural.HIGD1B.NDUFA4L2 | SEPT4 | 0.76 | CD.ILC.KRT81.IL22 | KRT86 | 2.13 |
| CD.B.HCK.DUSP4 | DUSP4 | 1.45 | CD.Endth/Mural.HIGD1B.NDUFA4L2 | HEY1 | 0.76 | CD.ILC.KRT81.IL22 | KLRC1 | 2.06 |
| CD.B.HCK.DUSP4 | SPIB | 1.40 | CD.Endth/Mural.HIGD1B.NDUFA4L2 | PTPRG | 0.75 | CD.ILC.KRT81.IL22 | KRT81 | 1.92 |
| CD.B.HCK.DUSP4 | BANK1 | 1.36 | CD.Endth/Mural.HIGD1B.NDUFA4L2 | ROBO4 | 0.75 | CD.ILC.KRT81.IL22 | LINC00299 | 0.86 |
| CD.B.HCK.DUSP4 | FCRL4 | 1.28 | CD.Endth/Mural.HIGD1B.NDUFA4L2 | ADAMTS1 | 0.74 | CD.ILC.KRT81.IL22 | NCR2 | 0.80 |
| CD.B.HCK.DUSP4 | FCRLA | 1.27 | CD.Endth/Mural.HIGD1B.NDUFA4L2 | BTNL9 | 0.71 | CD.ILC.KRT81.IL22 | IL23R | 1.14 |
| CD.B.HCK.DUSP4 | TNFRSF13B | 1.25 | CD.Endth/Mural.HIGD1B.NDUFA4L2 | THSD7A | 0.70 | CD.ILC.LST1.AREG | ALDOC | 2.12 |
| CD.B.HCK.DUSP4 | CLECL1 | 1.24 | CD.Endth/Mural.HIGD1B.NDUFA4L2 | TEK | 0.69 | CD.ILC.LST1.AREG | TRDC | 1.94 |
| CD.B.HCK.DUSP4 | FGR | 1.08 | CD.Endth/Mural.HIGD1B.NDUFA4L2 | EDNRA | 0.69 | CD.ILC.LST1.AREG | KRT86 | 1.91 |
| CD.B.HCK.DUSP4 | OSTN-AS1 | 0.92 | CD.Endth/Mural.HIGD1B.NDUFA4L2 | SHE | 0.66 | CD.ILC.LST1.AREG | KRT81 | 1.68 |
| CD.B.HCK.DUSP4 | CD82 | 1.23 | CD.Endth/Mural.HIGD1B.NDUFA4L2 | PALMD | 0.62 | CD.ILC.LST1.AREG | PCDH9 | 1.17 |
| CD.B.HCK.DUSP4 | IFI44L | 1.18 | CD.Endth/Mural.HIGD1B.NDUFA4L2 | ERG | 0.62 | CD.Mac.AIF1.HBEGF | FCN1 | 1.98 |
| CD.B.HCK.DUSP4 | CD22 | 1.04 | CD.Endth/Mural.HIGD1B.NDUFA4L2 | RASIP1 | 0.60 | CD.Mac.APOE.PTGDS | C1QB | 3.33 |
| CD.B.HCK.DUSP4 | SMIM14 | 1.66 | CD.Endth/Mural.HIGD1B.NDUFA4L2 | FAM162B | 0.60 | CD.Mac.APOE.PTGDS | C1QA | 3.22 |
| CD.B.IFI44L.ISG15 | VPREB3 | 1.89 | CD.Endth/Mural.HIGD1B.NDUFA4L2 | CXorf36 | 0.59 | CD.Mac.APOE.PTGDS | APOE | 3.10 |
| CD.B.IFI44L.ISG15 | HLA-DQA2 | 1.71 | CD.Endth/Mural.HIGD1B.NDUFA4L2 | NRN1 | 0.58 | CD.Mac.APOE.PTGDS | C1QC | 3.04 |
| CD.B.IFI44L.ISG15 | IGHD | 1.48 | CD.Endth/Mural.HIGD1B.NDUFA4L2 | STC1 | 0.58 | CD.Mac.APOE.PTGDS | SEPP1 | 2.98 |
| CD.B.IFI44L.ISG15 | TCL1A | 1.42 | CD.Endth/Mural.HIGD1B.NDUFA4L2 | ADCY4 | 0.57 | CD.Mac.APOE.PTGDS | APOC1 | 2.77 |
| CD.B.IGHA2.JCHAIN | CD79A | 1.56 | CD.Endth/Mural.HIGD1B.NDUFA4L2 | FZD4 | 0.57 | CD.Mac.APOE.PTGDS | LYZ | 2.41 |
| CD.B.IGHA2.JCHAIN | VPREB3 | 1.54 | CD.Endth/Mural.HIGD1B.NDUFA4L2 | AIF1L | 0.57 | CD.Mac.APOE.PTGDS | SLC40A1 | 2.38 |
| CD.B.IGHA2.JCHAIN | ARHGAP24 | 1.28 | CD.Endth/Mural.HIGD1B.NDUFA4L2 | EXOC3L2 | 0.54 | CD.Mac.APOE.PTGDS | DNASE1L3 | 2.36 |
| CD.B.IGHA2.JCHAIN | CXCR4 | 1.26 | CD.Endth/Mural.HIGD1B.NDUFA4L2 | GJC1 | 0.53 | CD.Mac.APOE.PTGDS | CST3 | 2.34 |
| CD.B.IGHA2.JCHAIN | CD37 | 1.23 | CD.Endth/Mural.HIGD1B.NDUFA4L2 | SOX7 | 0.52 | CD.Mac.APOE.PTGDS | FTL | 2.33 |
| CD.B.IGHA2.JCHAIN | RPL9 | 1.11 | CD.Endth/Mural.HIGD1B.NDUFA4L2 | KCNJ8 | 0.50 | CD.Mac.APOE.PTGDS | CCL18 | 2.32 |
| CD.B.IGHA2.JCHAIN | LINC00926 | 1.09 | CD.Endth/Mural.HIGD1B.NDUFA4L2 | NOVA2 | 0.45 | CD.Mac.APOE.PTGDS | TYROBP | 2.27 |
| CD.B.IGHA2.JCHAIN | CD79B | 1.07 | CD.Endth/Mural.HIGD1B.NDUFA4L2 | ABCC9 | 0.37 | CD.Mac.APOE.PTGDS | PSAP | 2.25 |
| CD.B.IGHA2.JCHAIN | CD83 | 1.04 | CD.Endth/Mural.HIGD1B.NDUFA4L2 | GUCY1A2 | 0.35 | CD.Mac.APOE.PTGDS | AIF1 | 2.24 |
| CD.B.IGHA2.JCHAIN | HLA-DRA | 1.02 | CD.Endth/Mural.HIGD1B.NDUFA4L2 | TNS1 | 0.64 | CD.Mac.APOE.PTGDS | MMP12 | 2.21 |
| CD.B.IGHA2.JCHAIN | EEF1B2 | 0.99 | CD.Endth/Mural.HIGD1B.NDUFA4L2 | MKL2 | 0.89 | CD.Mac.APOE.PTGDS | CTSC | 2.15 |
| CD.B.IGHA2.JCHAIN | HLA-DPA1 | 0.95 | CD.Endth/Mural.HIGD1B.NDUFA4L2 | ITGA1 | 1.41 | CD.Mac.APOE.PTGDS | FUCA1 | 2.15 |
| CD.B.IGHA2.JCHAIN | RPS3A | 0.93 | CD.Endth/Mural.HIGD1B.NDUFA4L2 | TGFBR2 | 2.10 | CD.Mac.APOE.PTGDS | FCER1G | 2.15 |
| CD.B.IGHA2.JCHAIN | NACA | 0.91 | CD.Endth/Mural.HIGD1B.NDUFA4L2 | RAI14 | 0.65 | CD.Mac.APOE.PTGDS | HLA-DRB1 | 2.15 |
| CD.B.IGHA2.JCHAIN | MS4A1 | 0.88 | CD.Endth/Mural.HIGD1B.NDUFA4L2 | NKX2-3 | 0.90 | CD.Mac.APOE.PTGDS | CTSD | 2.15 |
| CD.B.IGHA2.JCHAIN | EEF1A1 | 0.87 | CD.Endth/Mural.HIGD1B.NDUFA4L2 | LINC01235 | 0.62 | CD.Mac.APOE.PTGDS | CD68 | 2.11 |
| CD.B.IGHA2.JCHAIN | RPS27A | 0.80 | CD.Endth/Mural.HIGD1B.NDUFA4L2 | TMEM47 | 0.77 | CD.Mac.APOE.PTGDS | MS4A6A | 2.11 |
| CD.B.IGHA2.JCHAIN | TPT1 | 0.80 | CD.Endth/Mural.HIGD1B.NDUFA4L2 | TPPP3 | 0.90 | CD.Mac.APOE.PTGDS | IGSF6 | 2.07 |
| CD.B.IGHA2.JCHAIN | RPL10 | 0.79 | CD.Endth/Mural.HIGD1B.NDUFA4L2 | PTPRM | 0.64 | CD.Mac.APOE.PTGDS | CTSB | 2.07 |
| CD.B.IGHA2.JCHAIN | RPS23 | 0.77 | CD.Endth/Mural.HIGD1B.NDUFA4L2 | CRIM1 | 1.02 | CD.Mac.APOE.PTGDS | NPC2 | 2.03 |
| CD.B.IGHA2.JCHAIN | HLA-DPB1 | 0.77 | CD.Endth/Mural.HIGD1B.NDUFA4L2 | ARHGEF15 | 0.48 | CD.Mac.APOE.PTGDS | HLA-DPA1 | 2.03 |
| CD.B.IGHA2.JCHAIN | RPS5 | 0.74 | CD.Endth/Mural.HIGD1B.NDUFA4L2 | IGFBP7 | 2.92 | CD.Mac.APOE.PTGDS | HLA-DRA | 1.99 |
| CD.B.IGHA2.JCHAIN | RPL7 | 0.73 | CD.Endth/Mural.HIGD1B.NDUFA4L2 | ANO1 | 0.27 | CD.Mac.APOE.PTGDS | CTSS | 1.98 |
| CD.B.IGHA2.JCHAIN | RPS13 | 0.71 | CD.Endth/Mural.HIGD1B.NDUFA4L2 | HSPA12B | 0.48 | CD.Mac.APOE.PTGDS | MMP9 | 1.98 |
| CD.B.IGHA2.JCHAIN | RPL30 | 0.68 | CD.Endth/Mural.HIGD1B.NDUFA4L2 | KIAA1462 | 0.42 | CD.Mac.APOE.PTGDS | GPNMB | 1.97 |
| CD.B.IGHA2.JCHAIN | RPL18A | 0.63 | CD.Endth/Mural.HIGD1B.NDUFA4L2 | IGFBP6 | 1.31 | CD.Mac.APOE.PTGDS | HLA-DQA1 | 1.97 |
| CD.B.IGHA2.JCHAIN | RPL13A | 0.59 | CD.Endth/Mural.HIGD1B.NDUFA4L2 | EPS8 | 1.04 | CD.Mac.APOE.PTGDS | LIPA | 1.96 |
| CD.B.IGHA2.JCHAIN | RPS6 | 0.57 | CD.Endth/Mural.HIGD1B.NDUFA4L2 | EGFLAM | 0.34 | CD.Mac.APOE.PTGDS | ACP5 | 1.96 |
| CD.B.IGHA2.JCHAIN | RPL32 | 0.56 | CD.Endth/Mural.HIGD1B.NDUFA4L2 | FXYD6 | 0.84 | CD.Mac.APOE.PTGDS | GRN | 1.90 |
| CD.B.IGHA2.JCHAIN | RPL13 | 0.56 | CD.Endth/Mural.HIGD1B.NDUFA4L2 | HRC | 0.29 | CD.Mac.APOE.PTGDS | FCGRT | 1.78 |
| CD.B.IGHA2.JCHAIN | RPS12 | 0.62 | CD.Endth/Ven.ACKR1.ADGRG6 | ACKR1 | 3.45 | CD.Mac.APOE.PTGDS | HLA-DPB1 | 1.78 |
| CD.B.IGHA2.JCHAIN | RPSA | 0.77 | CD.Endth/Ven.ACKR1.ADGRG6 | CLDN5 | 2.93 | CD.Mac.APOE.PTGDS | CD14 | 1.76 |
| CD.B.IGHG1.IGLC2 | CD74 | 1.12 | CD.Endth/Ven.ACKR1.ADGRG6 | VWF | 2.88 | CD.Mac.APOE.PTGDS | LGMN | 1.74 |
| CD.B.IGHG1.IGLC2 | RPS27 | 0.76 | CD.Endth/Ven.ACKR1.ADGRG6 | RAMP3 | 2.24 | CD.Mac.APOE.PTGDS | MS4A7 | 1.73 |
| CD.B.IGHG1.IGLC2 | HLA-DRB1 | 0.98 | CD.Endth/Ven.ACKR1.ADGRG6 | TSPAN7 | 2.18 | CD.Mac.APOE.PTGDS | PLD3 | 1.72 |
| CD.B.IGHG1.IGLC2 | IGHD | 1.40 | CD.Endth/Ven.ACKR1.ADGRG6 | MADCAM1 | 2.08 | CD.Mac.APOE.PTGDS | ATOX1 | 1.71 |
| CD.B.IGLC2.HLA-DRB1 | MS4A1 | 1.34 | CD.Endth/Ven.ACKR1.ADGRG6 | CPE | 2.02 | CD.Mac.APOE.PTGDS | CPVL | 1.70 |
| CD.B.IGLC2.HLA-DRB1 | VPREB3 | 1.37 | CD.Endth/Ven.ACKR1.ADGRG6 | LIFR | 1.95 | CD.Mac.APOE.PTGDS | HLA-DRB5 | 1.67 |
| CD.B.IGLC3.IGLC2 | IGHD | 1.72 | CD.Endth/Ven.ACKR1.ADGRG6 | PLVAP | 1.92 | CD.Mac.APOE.PTGDS | FGL2 | 1.65 |
| CD.B.IGLC3.IGLC2 | TCL1A | 1.57 | CD.Endth/Ven.ACKR1.ADGRG6 | JAM2 | 1.89 | CD.Mac.APOE.PTGDS | MS4A4A | 1.65 |
| CD.B.IGLC3.IGLC2 | IGLC3 | 0.27 | CD.Endth/Ven.ACKR1.ADGRG6 | EGFL7 | 1.81 | CD.Mac.APOE.PTGDS | PLA2G7 | 1.65 |
| CD.B.IGLC3.IGLC2 | MS4A1 | 1.52 | CD.Endth/Ven.ACKR1.ADGRG6 | IL33 | 1.65 | CD.Mac.APOE.PTGDS | TMEM176B | 1.65 |
| CD.B.MT-CO2.MT-CO1 | MS4A1 | 1.59 | CD.Endth/Ven.ACKR1.ADGRG6 | SNCG | 1.51 | CD.Mac.APOE.PTGDS | CTSL | 1.61 |
| CD.B.MT-CO2.MT-CO1 | CD83 | 1.37 | CD.Endth/Ven.ACKR1.ADGRG6 | ADGRG6 | 1.37 | CD.Mac.APOE.PTGDS | HLA-DMA | 1.60 |
| CD.B.MT-CO2.MT-CO1 | VPREB3 | 1.34 | CD.Endth/Ven.ACKR1.ADGRG6 | ZNF385D | 1.36 | CD.Mac.APOE.PTGDS | PRDX1 | 1.57 |
| CD.B.MT-CO2.MT-CO1 | CD79A | 1.33 | CD.Endth/Ven.ACKR1.ADGRG6 | SELP | 1.33 | CD.Mac.APOE.PTGDS | HLA-DQB1 | 1.57 |
| CD.B.MT-CO2.MT-CO1 | BANK1 | 1.20 | CD.Endth/Ven.ACKR1.ADGRG6 | SELE | 1.24 | CD.Mac.APOE.PTGDS | ASAH1 | 1.56 |
| CD.B.MT-CO2.MT-CO1 | CD79B | 1.13 | CD.Endth/Ven.ACKR1.ADGRG6 | MMRN1 | 1.23 | CD.Mac.APOE.PTGDS | CD74 | 1.56 |
| CD.B.MT-CO2.MT-CO1 | HLA-DRB1 | 1.02 | CD.Endth/Ven.ACKR1.ADGRG6 | CCL14 | 1.15 | CD.Mac.APOE.PTGDS | CTSZ | 1.56 |
| CD.B.MT-CO2.MT-CO1 | CXCR4 | 1.02 | CD.Endth/Ven.ACKR1.ADGRG6 | TLL1 | 1.10 | CD.Mac.APOE.PTGDS | HLA-DMB | 1.55 |
| CD.B.MT-CO2.MT-CO1 | CD74 | 0.99 | CD.Endth/Ven.ACKR1.ADGRG6 | RAB3C | 0.96 | CD.Mac.APOE.PTGDS | SAT1 | 1.54 |
| CD.B.MT-CO2.MT-CO1 | HLA-DQA1 | 0.91 | CD.Endth/Ven.ACKR1.ADGRG6 | APLNR | 1.59 | CD.Mac.APOE.PTGDS | GPX1 | 1.51 |
| CD.B.MT-CO2.MT-CO1 | CD52 | 0.89 | CD.Endth/Ven.ACKR1.ADGRG6 | PCAT19 | 1.41 | CD.Mac.APOE.PTGDS | CD63 | 1.50 |
| CD.B.MT-CO2.MT-CO1 | HLA-DRA | 0.77 | CD.Endth/Ven.ACKR1.ADGRG6 | MGP | 2.09 | CD.Mac.APOE.PTGDS | RP11- | 1.49 |
| | | | | | | | 1143G9.4 | |
| CD.B.MT-CO2.MT-CO1 | RPL18A | 0.57 | CD.Endth/Ven.ACKR1.ADGRG6 | LEPR | 1.46 | CD.Mac.APOE.PTGDS | CAPG | 1.43 |
| CD.B.MT-CO2.MT-CO1 | HLA-DPB1 | 0.55 | CD.Endth/Ven.ADGRG6.ACKR1 | ACKR1 | 3.72 | CD.Mac.APOE.PTGDS | ITM2B | 1.42 |
| CD.B.MT-CO2.MT-CO1 | HLA-DQB1 | 0.87 | CD.Endth/Ven.ADGRG6.ACKR1 | CLDN5 | 3.24 | CD.Mac.APOE.PTGDS | CXCL9 | 1.40 |
| CD.B.MT-CO2.MT-CO1 | BTG1 | 0.77 | CD.Endth/Ven.ADGRG6.ACKR1 | TSPAN7 | 2.44 | CD.Mac.APOE.PTGDS | FOLR2 | 1.39 |
| CD.B.MT-CO2.MT-CO1 | IGHD | 1.10 | CD.Endth/Ven.ADGRG6.ACKR1 | RAMP3 | 2.44 | CD.Mac.APOE.PTGDS | FAM26F | 1.37 |
| CD.B.MT-CO2.MT-CO1 | CD37 | 0.94 | CD.Endth/Ven.ADGRG6.ACKR1 | RAMP2 | 2.39 | CD.Mac.APOE.PTGDS | CXCL8 | 1.36 |
| CD.B.MT-ND4L.IGHG1 | IGHD | 1.94 | CD.Endth/Ven.ADGRG6.ACKR1 | CPE | 2.33 | CD.Mac.APOE.PTGDS | CTSH | 1.34 |
| CD.B.MT-ND4L.IGHG1 | TCL1A | 1.61 | CD.Endth/Ven.ADGRG6.ACKR1 | VWF | 2.20 | CD.Mac.APOE.PTGDS | GLUL | 1.32 |
| CD.B.MT-ND4L.IGHG1 | MS4A1 | 1.59 | CD.Endth/Ven.ADGRG6.ACKR1 | JAM2 | 2.09 | CD.Mac.APOE.PTGDS | LY96 | 1.32 |
| CD.B.MT-ND4L.IGHG1 | CD79B | 1.43 | CD.Endth/Ven.ADGRG6.ACKR1 | MADCAM1 | 2.08 | CD.Mac.APOE.PTGDS | LST1 | 1.31 |
| CD.B.MT-ND4L.IGHG1 | CD79A | 1.40 | CD.Endth/Ven.ADGRG6.ACKR1 | PLVAP | 2.08 | CD.Mac.APOE.PTGDS | SOD2 | 1.30 |
| CD.B.MT-ND4L.IGHG1 | LINC00926 | 1.39 | CD.Endth/Ven.ADGRG6.ACKR1 | FAM167B | 1.94 | CD.Mac.APOE.PTGDS | CREG1 | 1.29 |
| CD.B.MT-ND4L.IGHG1 | FCER2 | 1.38 | CD.Endth/Ven.ADGRG6.ACKR1 | CCL14 | 1.84 | CD.Mac.APOE.PTGDS | FTH1 | 1.29 |
| CD.B.MT-ND4L.IGHG1 | MEF2C | 1.37 | CD.Endth/Ven.ADGRG6.ACKR1 | ADGRG6 | 1.77 | CD.Mac.APOE.PTGDS | S100A11 | 1.28 |
| CD.B.MT-ND4L.IGHG1 | BANK1 | 1.35 | CD.Endth/Ven.ADGRG6.ACKR1 | IL33 | 1.69 | CD.Mac.APOE.PTGDS | C1orf54 | 1.27 |
| CD.B.MT-ND4L.IGHG1 | SELL | 1.33 | CD.Endth/Ven.ADGRG6.ACKR1 | PCAT19 | 1.68 | CD.Mac.APOE.PTGDS | IL18 | 1.25 |
| CD.B.MT-ND4L.IGHG1 | HLA-DQB1 | 1.29 | CD.Endth/Ven.ADGRG6.ACKR1 | EGFL7 | 1.66 | CD.Mac.APOE.PTGDS | CCL3 | 1.24 |
| CD.B.MT-ND4L.IGHG1 | CD37 | 1.26 | CD.Endth/Ven.ADGRG6.ACKR1 | ECSCR-1 | 1.66 | CD.Mac.APOE.PTGDS | LILRB4 | 1.23 |
| CD.B.MT-ND4L.IGHG1 | VPREB3 | 1.24 | CD.Endth/Ven.ADGRG6.ACKR1 | LIFR | 1.65 | CD.Mac.APOE.PTGDS | RNASE6 | 1.23 |
| CD.B.MT-ND4L.IGHG1 | HLA-DRB5 | 1.24 | CD.Endth/Ven.ADGRG6.ACKR1 | APLNR | 1.43 | CD.Mac.APOE.PTGDS | RNASET2 | 1.22 |
| CD.B.MT-ND4L.IGHG1 | CD74 | 1.18 | CD.Endth/Ven.ADGRG6.ACKR1 | NRN1 | 1.37 | CD.Mac.APOE.PTGDS | PLA2G2D | 1.21 |
| CD.B.MT-ND4L.IGHG1 | CD83 | 1.17 | CD.Endth/Ven.ADGRG6.ACKR1 | EMCN | 1.31 | CD.Mac.APOE.PTGDS | CYBB | 1.18 |
| CD.B.MT-ND4L.IGHG1 | CD52 | 1.10 | CD.Endth/Ven.ADGRG6.ACKR1 | SNCG | 1.31 | CD.Mac.APOE.PTGDS | BRI3 | 1.15 |
| CD.B.MT-ND4L.IGHG1 | HLA-DRA | 0.88 | CD.Endth/Ven.ADGRG6.ACKR1 | ZNF385D | 1.30 | CD.Mac.APOE.PTGDS | VSIG4 | 1.14 |
| CD.B.MT-ND4L.IGHG1 | RPS26 | 0.83 | CD.Endth/Ven.ADGRG6.ACKR1 | SELP | 1.29 | CD.Mac.APOE.PTGDS | IL1B | 1.14 |
| CD.B.MT-ND4L.IGHG1 | CXCR4 | 0.81 | CD.Endth/Ven.ADGRG6.ACKR1 | ESAM | 1.27 | CD.Mac.APOE.PTGDS | TMEM176A | 1.14 |
| CD.B.MT-ND4L.IGHG1 | HLA-DPA1 | 0.76 | CD.Endth/Ven.ADGRG6.ACKR1 | MMRN2 | 1.22 | CD.Mac.APOE.PTGDS | FCGR3A | 1.13 |
| CD.B.MT-ND4L.IGHG1 | RPS27 | 0.66 | CD.Endth/Ven.ADGRG6.ACKR1 | PALMD | 1.19 | CD.Mac.APOE.PTGDS | ATP6V1F | 1.12 |
| CD.B.MT-ND4L.IGHG1 | HLA-DPB1 | 0.64 | CD.Endth/Ven.ADGRG6.ACKR1 | CYYR1 | 1.17 | CD.Mac.APOE.PTGDS | SPI1 | 1.11 |
| CD.B.MT-ND4L.IGHG1 | RPL18A | 0.64 | CD.Endth/Ven.ADGRG6.ACKR1 | RAB3C | 1.09 | CD.Mac.APOE.PTGDS | CSTA | 1.11 |
| CD.B.MT-ND4L.IGHG1 | RPL34 | 0.47 | CD.Endth/Ven.ADGRG6.ACKR1 | MMRN1 | 1.06 | CD.Mac.APOE.PTGDS | VAMP8 | 1.11 |
| CD.B.MT-ND4L.IGHG1 | RPL39 | 0.47 | CD.Endth/Ven.ADGRG6.ACKR1 | MYCT1 | 0.99 | CD.Mac.APOE.PTGDS | ENPP2 | 1.10 |
| CD.B.MT-ND4L.IGHG1 | HLA-DRB1 | 0.45 | CD.Endth/Ven.ADGRG6.ACKR1 | TEK | 0.93 | CD.Mac.APOE.PTGDS | PLAUR | 1.10 |
| CD.B.MT-ND4L.IGHG1 | RPL21 | 0.43 | CD.Endth/Ven.ADGRG6.ACKR1 | FAM110D | 0.92 | CD.Mac.APOE.PTGDS | C2 | 1.10 |
| CD.B.MT-ND4L.IGHG1 | HLA-DQA1 | 0.77 | CD.Endth/Ven.ADGRG6.ACKR1 | CADM3-AS1 | 0.63 | CD.Mac.APOE.PTGDS | MPEG1 | 1.09 |
| CD.B.MT-ND4L.IGHG1 | HVCN1 | 1.32 | CD.Endth/Ven.ADGRG6.ACKR1 | LHX6 | 0.65 | CD.Mac.APOE.PTGDS | RNF130 | 1.07 |
| CD.B.MT-ND4L.IGHG1 | CD69 | 0.71 | CD.Endth/Ven.ADGRG6.ACKR1 | CALCRL | 1.59 | CD.Mac.APOE.PTGDS | ITGB2 | 1.07 |
| CD.B.MT-ND4L.IGHG1 | CD22 | 1.21 | CD.Endth/Ven.ADGRG6.ACKR1 | ADGRL4 | 1.08 | CD.Mac.APOE.PTGDS | CSF1R | 1.05 |
| CD.B.MT-ND4L.IGHG1 | RP11- | 1.19 | CD.Endth/Ven.ADGRG6.ACKR1 | LMCD1 | 1.39 | CD.Mac.APOE.PTGDS | CYBA | 1.05 |
| | 693J15.5 | | | | | | | |
| CD.B.RPS10.FCER2 | MS4A1 | 1.64 | CD.Endth/Ven.ADGRG6.ACKR1 | CLEC14A | 1.60 | CD.Mac.APOE.PTGDS | PPT1 | 1.04 |
| CD.B.RPS10.FCER2 | CD79A | 1.52 | CD.Endth/Ven.ADGRG6.ACKR1 | CDH5 | 0.98 | CD.Mac.APOE.PTGDS | JAML | 1.03 |
| CD.B.RPS10.FCER2 | VPREB3 | 1.40 | CD.Endth/Ven.CCL23.ACKR1 | ACKR1 | 3.69 | CD.Mac.APOE.PTGDS | AXL | 1.03 |
| CD.B.RPS10.FCER2 | TCL1A | 1.33 | CD.Endth/Ven.CCL23.ACKR1 | CLDN5 | 2.64 | CD.Mac.APOE.PTGDS | DAB2 | 1.02 |
| CD.B.RPS10.FCER2 | IGHD | 1.30 | CD.Endth/Ven.CCL23.ACKR1 | MGP | 2.04 | CD.Mac.APOE.PTGDS | CLEC10A | 1.01 |
| CD.B.RPS10.FCER2 | TNFRSF13C | 1.19 | CD.Endth/Ven.CCL23.ACKR1 | PLVAP | 1.97 | CD.Mac.APOE.PTGDS | TNFSF13B | 1.01 |
| CD.B.RPS10.FCER2 | CD37 | 1.17 | CD.Endth/Ven.CCL23.ACKR1 | TSPAN7 | 1.82 | CD.Mac.APOE.PTGDS | MRC1 | 1.00 |
| CD.B.RPS10.FCER2 | CD22 | 1.13 | CD.Endth/Ven.CCL23.ACKR1 | CPE | 1.77 | CD.Mac.APOE.PTGDS | SERPING1 | 1.00 |
| CD.B.RPS10.FCER2 | MEF2C | 1.13 | CD.Endth/Ven.CCL23.ACKR1 | VWF | 1.76 | CD.Mac.APOE.PTGDS | CTSA | 1.00 |
| CD.B.RPS10.FCER2 | FCER2 | 1.11 | CD.Endth/Ven.CCL23.ACKR1 | RAMP3 | 1.65 | CD.Mac.APOE.PTGDS | CCL4L2 | 1.00 |
| CD.B.RPS10.FCER2 | CD79B | 1.11 | CD.Endth/Ven.CCL23.ACKR1 | JAM2 | 1.63 | CD.Mac.APOE.PTGDS | ANXA5 | 0.99 |
| CD.B.RPS10.FCER2 | CD74 | 1.09 | CD.Endth/Ven.CCL23.ACKR1 | CCL14 | 1.55 | CD.Mac.APOE.PTGDS | HNMT | 0.99 |
| CD.B.RPS10.FCER2 | RPS10 | 1.09 | CD.Endth/Ven.CCL23.ACKR1 | FAM167B | 1.54 | CD.Mac.APOE.PTGDS | CXCL2 | 0.99 |
| CD.B.RPS10.FCER2 | CD83 | 1.05 | CD.Endth/Ven.CCL23.ACKR1 | CLEC14A | 1.52 | CD.Mac.APOE.PTGDS | AOAH | 0.98 |
| CD.B.RPS10.FCER2 | HVCN1 | 1.03 | CD.Endth/Ven.CCL23.ACKR1 | CCL23 | 1.52 | CD.Mac.APOE.PTGDS | CD163 | 0.98 |
| CD.B.RPS10.FCER2 | PHACTR1 | 1.03 | CD.Endth/Ven.CCL23.ACKR1 | SNCG | 1.40 | CD.Mac.APOE.PTGDS | LAP3 | 0.98 |
| CD.B.RPS10.FCER2 | BANK1 | 0.99 | CD.Endth/Ven.CCL23.ACKR1 | PCAT19 | 1.31 | CD.Mac.APOE.PTGDS | SERPINF1 | 0.98 |
| CD.B.RPS10.FCER2 | CD72 | 0.98 | CD.Endth/Ven.CCL23.ACKR1 | ECSCR-1 | 1.29 | CD.Mac.APOE.PTGDS | TYMP | 0.97 |
| CD.B.RPS10.FCER2 | CD52 | 0.96 | CD.Endth/Ven.CCL23.ACKR1 | SMAD1 | 1.28 | CD.Mac.APOE.PTGDS | SCPEP1 | 0.97 |
| CD.B.RPS10.FCER2 | RP11- | 0.93 | CD.Endth/Ven.CCL23.ACKR1 | NRN1 | 1.26 | CD.Mac.APOE.PTGDS | ATP6AP2 | 0.97 |
| | 231C14.7 | | | | | | | |
| CD.B.RPS10.FCER2 | HLA-DMB | 0.90 | CD.Endth/Ven.CCL23.ACKR1 | ADGRL4 | 1.23 | CD.Mac.APOE.PTGDS | MFSD1 | 0.97 |
| CD.B.RPS10.FCER2 | HLA-DQB1 | 0.83 | CD.Endth/Ven.CCL23.ACKR1 | IL33 | 1.21 | CD.Mac.APOE.PTGDS | CCL3L3 | 0.96 |
| CD.B.RPS10.FCER2 | HLA-DPB1 | 0.82 | CD.Endth/Ven.CCL23.ACKR1 | MADCAM1 | 1.20 | CD.Mac.APOE.PTGDS | LAMP2 | 0.96 |
| CD.B.RPS10.FCER2 | CXCR4 | 0.81 | CD.Endth/Ven.CCL23.ACKR1 | ZNF385D | 1.11 | CD.Mac.APOE.PTGDS | HES1 | 0.96 |
| CD.B.RPS10.FCER2 | LAPTM5 | 0.81 | CD.Endth/Ven.CCL23.ACKR1 | MMRN1 | 1.11 | CD.Mac.APOE.PTGDS | RAC1 | 0.95 |
| CD.B.RPS10.FCER2 | HLA-DRA | 0.80 | CD.Endth/Ven.CCL23.ACKR1 | PCDH17 | 1.09 | CD.Mac.APOE.PTGDS | CLEC7A | 0.95 |
| CD.B.RPS10.FCER2 | HLA-DQA1 | 0.78 | CD.Endth/Ven.CCL23.ACKR1 | SOX18 | 1.07 | CD.Mac.APOE.PTGDS | TGFBI | 0.94 |
| CD.B.RPS10.FCER2 | RPS23 | 0.74 | CD.Endth/Ven.CCL23.ACKR1 | CD34 | 0.97 | CD.Mac.APOE.PTGDS | HEXB | 0.94 |
| CD.B.RPS10.FCER2 | RPS21 | 0.74 | CD.Endth/Ven.CCL23.ACKR1 | LEPR | 0.90 | CD.Mac.APOE.PTGDS | FPR3 | 0.93 |
| CD.B.RPS10.FCER2 | RPL18A | 0.73 | CD.Endth/Ven.CCL23.ACKR1 | MEOX1 | 0.84 | CD.Mac.APOE.PTGDS | AKR1B1 | 0.93 |
| CD.B.RPS10.FCER2 | RPLP2 | 0.72 | CD.Endth/Ven.CCL23.ACKR1 | SELP | 0.81 | CD.Mac.APOE.PTGDS | LGALS1 | 0.93 |
| CD.B.RPS10.FCER2 | HLA-DPA1 | 0.72 | CD.Endth/Ven.CCL23.ACKR1 | TEK | 0.69 | CD.Mac.APOE.PTGDS | SGK1 | 0.93 |
| CD.B.RPS10.FCER2 | BTG1 | 0.72 | CD.Endth/Ven.CCL23.ACKR1 | RASSF9 | 0.67 | CD.Mac.APOE.PTGDS | SDCBP | 0.92 |
| CD.B.RPS10.FCER2 | RPS5 | 0.71 | CD.Endth/Ven.CCL23.ACKR1 | MEOX2 | 0.55 | CD.Mac.APOE.PTGDS | ATP6VOB | 0.92 |
| CD.B.RPS10.FCER2 | RPSA | 0.70 | CD.Endth/Ven.CCL23.ACKR1 | MYCT1 | 0.75 | CD.Mac.APOE.PTGDS | CD86 | 0.92 |
| CD.B.RPS10.FCER2 | RPL36A | 0.70 | CD.Endth/Ven.CCL23.ACKR1 | CYYR1 | 0.97 | CD.Mac.APOE.PTGDS | BLVRB | 0.91 |
| CD.B.RPS10.FCER2 | RPS8 | 0.69 | CD.Endth/Ven.CCL23.ACKR1 | RAMP2 | 1.82 | CD.Mac.APOE.PTGDS | PLEK | 0.90 |
| CD.B.RPS10.FCER2 | RPS11 | 0.67 | CD.Endth/Ven.CCL23.ACKR1 | ERG | 0.76 | CD.Mac.APOE.PTGDS | AP2S1 | 0.90 |
| CD.B.RPS10.FCER2 | RPL8 | 0.67 | CD.Endth/Ven.CCL23.ACKR1 | IL3RA | 1.23 | CD.Mac.APOE.PTGDS | CXCL16 | 0.90 |
| CD.B.RPS10.FCER2 | HLA-DRB5 | 0.66 | CD.Endth/Ven.CCL23.ACKR1 | TLL1 | 0.57 | CD.Mac.APOE.PTGDS | IFI30 | 0.90 |
| CD.B.RPS10.FCER2 | RPS17 | 0.66 | CD.Endth/Ven.CCL23.ACKR1 | NPDC1 | 1.79 | CD.Mac.APOE.PTGDS | LGALS2 | 0.90 |
| CD.B.RPS10.FCER2 | RPL7A | 0.64 | CD.Endth/Ven.CPE.GALNT15 | ACKR1 | 3.48 | CD.Mac.APOE.PTGDS | VAMP5 | 0.90 |
| CD.B.RPS10.FCER2 | RPL10A | 0.63 | CD.Endth/Ven.CPE.GALNT15 | CLDN5 | 3.10 | CD.Mac.APOE.PTGDS | IFNGR1 | 0.88 |
| CD.B.RPS10.FCER2 | RPS27 | 0.63 | CD.Endth/Ven.CPE.GALNT15 | CPE | 2.82 | CD.Mac.APOE.PTGDS | GPR34 | 0.87 |
| CD.B.RPS10.FCER2 | RPL26 | 0.63 | CD.Endth/Ven.CPE.GALNT15 | JAM2 | 2.28 | CD.Mac.APOE.PTGDS | UBE2D1 | 0.87 |
| CD.B.RPS10.FCER2 | RPL5 | 0.63 | CD.Endth/Ven.CPE.GALNT15 | MADCAM1 | 1.82 | CD.Mac.APOE.PTGDS | MGST2 | 0.87 |
| CD.B.RPS10.FCER2 | RPL37 | 0.62 | CD.Endth/Ven.CPE.GALNT15 | PCAT19 | 1.77 | CD.Mac.APOE.PTGDS | OAZ2 | 0.86 |
| CD.B.RPS10.FCER2 | RPL30 | 0.61 | CD.Endth/Ven.CPE.GALNT15 | APLNR | 1.50 | CD.Mac.APOE.PTGDS | C3AR1 | 0.85 |
| CD.B.RPS10.FCER2 | RPL23A | 0.61 | CD.Endth/Ven.CPE.GALNT15 | IL33 | 1.46 | CD.Mac.APOE.PTGDS | PYCARD | 0.85 |
| CD.B.RPS10.FCER2 | RPL41 | 0.61 | CD.Endth/Ven.CPE.GALNT15 | CYYR1 | 1.41 | CD.Mac.APOE.PTGDS | MARCKS | 0.85 |
| CD.B.RPS10.FCER2 | RPL37A | 0.61 | CD.Endth/Ven.CPE.GALNT15 | ZNF385D | 1.36 | CD.Mac.APOE.PTGDS | NR1H3 | 0.84 |
| CD.B.RPS10.FCER2 | RPL23 | 0.60 | CD.Endth/Ven.CPE.GALNT15 | PTPRB | 1.32 | CD.Mac.APOE.PTGDS | HEXA | 0.84 |
| CD.B.RPS10.FCER2 | RPL27 | 0.60 | CD.Endth/Ven.CPE.GALNT15 | NRN1 | 1.32 | CD.Mac.APOE.PTGDS | ATP6V1B2 | 0.83 |
| CD.B.RPS10.FCER2 | FAU | 0.60 | CD.Endth/Ven.CPE.GALNT15 | CCL14 | 1.31 | CD.Mac.APOE.PTGDS | NINJ1 | 0.83 |
| CD.B.RPS10.FCER2 | GNB2L1 | 0.59 | CD.Endth/Ven.CPE.GALNT15 | MYCT1 | 1.15 | CD.Mac.APOE.PTGDS | HCK | 0.83 |
| CD.B.RPS10.FCER2 | RPL19 | 0.59 | CD.Endth/Ven.CPE.GALNT15 | NTN4 | 1.01 | CD.Mac.APOE.PTGDS | RGS10 | 0.82 |
| CD.B.RPS10.FCER2 | RPL9 | 0.56 | CD.Endth/Ven.CPE.GALNT15 | TLL1 | 1.01 | CD.Mac.APOE.PTGDS | SLC7A7 | 0.81 |
| CD.B.RPS10.FCER2 | RPL15 | 0.56 | CD.Endth/Ven.CPE.GALNT15 | RAB3C | 0.83 | CD.Mac.APOE.PTGDS | P2RY13 | 0.81 |
| CD.B.RPS10.FCER2 | RPS12 | 0.56 | CD.Endth/Ven.CPE.GALNT15 | FAM155A | 0.76 | CD.Mac.APOE.PTGDS | NPL | 0.80 |
| CD.B.RPS10.FCER2 | RPL38 | 0.55 | CD.Endth/Ven.CPE.GALNT15 | C1QTNF9 | 0.73 | CD.Mac.APOE.PTGDS | IL411 | 0.80 |
| CD.B.RPS10.FCER2 | RPL18 | 0.54 | CD.Endth/Ven.CPE.GALNT15 | GALNT15 | 0.64 | CD.Mac.APOE.PTGDS | BLVRA | 0.80 |
| CD.B.RPS10.FCER2 | RPL13A | 0.54 | CD.Endth/Ven.CPE.GALNT15 | LIFR | 1.75 | CD.Mac.APOE.PTGDS | TSPAN4 | 0.80 |
| CD.B.RPS10.FCER2 | RPL12 | 0.53 | CD.Endth/Ven.CPE.GALNT15 | SNCG | 1.41 | CD.Mac.APOE.PTGDS | STAB1 | 0.79 |
| CD.B.RPS10.FCER2 | RPL29 | 0.53 | CD.Endth/Ven.CPE.GALNT15 | BCAM | 1.63 | CD.Mac.APOE.PTGDS | MAFB | 0.79 |
| CD.B.RPS10.FCER2 | RPS16 | 0.53 | CD.Endth/Ven.CPE.GALNT15 | KLK10 | 0.39 | CD.Mac.APOE.PTGDS | SMCO4 | 0.78 |
| CD.B.RPS10.FCER2 | RPS2 | 0.53 | CD.Endth/Ven.CPE.GALNT15 | ADGRL4 | 1.25 | CD.Mac.APOE.PTGDS | CD36 | 0.77 |
| CD.B.RPS10.FCER2 | RPS20 | 0.52 | CD.Endth/Ven.LAMP3.LIPG | VWF | 3.11 | CD.Mac.APOE.PTGDS | NAGK | 0.77 |
| CD.B.RPS10.FCER2 | RPL11 | 0.52 | CD.Endth/Ven.LAMP3.LIPG | PLVAP | 3.08 | CD.Mac.APOE.PTGDS | TPP1 | 0.77 |
| CD.B.RPS10.FCER2 | RPS7 | 0.52 | CD.Endth/Ven.LAMP3.LIPG | ENG | 3.00 | CD.Mac.APOE.PTGDS | SLAMF8 | 0.76 |
| CD.B.RPS10.FCER2 | RPL39 | 0.51 | CD.Endth/Ven.LAMP3.LIPG | MGP | 2.93 | CD.Mac.APOE.PTGDS | SAMHD1 | 0.76 |
| CD.B.RPS10.FCER2 | RPS25 | 0.51 | CD.Endth/Ven.LAMP3.LIPG | COL4A1 | 2.73 | CD.Mac.APOE.PTGDS | FCGR1A | 0.76 |
| CD.B.RPS10.FCER2 | RPS26 | 0.51 | CD.Endth/Ven.LAMP3.LIPG | SELE | 2.70 | CD.Mac.APOE.PTGDS | SLCO2B1 | 0.76 |
| CD.B.RPS10.FCER2 | RPL31 | 0.51 | CD.Endth/Ven.LAMP3.LIPG | HSPG2 | 2.64 | CD.Mac.APOE.PTGDS | TFRC | 0.75 |
| CD.B.RPS10.FCER2 | RPL13 | 0.51 | CD.Endth/Ven.LAMP3.LIPG | ACKR1 | 2.61 | CD.Mac.APOE.PTGDS | TCN2 | 0.75 |
| CD.B.RPS10.FCER2 | RPS19 | 0.49 | CD.Endth/Ven.LAMP3.LIPG | COL4A2 | 2.36 | CD.Mac.APOE.PTGDS | TMSB4X | 0.74 |
| CD.B.RPS10.FCER2 | RPL28 | 0.49 | CD.Endth/Ven.LAMP3.LIPG | RAMP3 | 2.35 | CD.Mac.APOE.PTGDS | MNDA | 0.74 |
| CD.B.RPS10.FCER2 | EEF1A1 | 0.49 | CD.Endth/Ven.LAMP3.LIPG | COL15A1 | 2.33 | CD.Mac.APOE.PTGDS | TBXAS1 | 0.73 |
| CD.B.RPS10.FCER2 | RPL6 | 0.48 | CD.Endth/Ven.LAMP3.LIPG | VWA1 | 2.33 | CD.Mac.APOE.PTGDS | CECR1 | 0.72 |
| CD.B.RPS10.FCER2 | RPL35A | 0.48 | CD.Endth/Ven.LAMP3.LIPG | PCAT19 | 2.22 | CD.Mac.APOE.PTGDS | HMOX1 | 0.71 |
| CD.B.RPS10.FCER2 | RPS18 | 0.48 | CD.Endth/Ven.LAMP3.LIPG | CRIP2 | 2.09 | CD.Mac.APOE.PTGDS | FCGR2A | 0.70 |
| CD.B.RPS10.FCER2 | RPL32 | 0.48 | CD.Endth/Ven.LAMP3.LIPG | RAMP2 | 2.03 | CD.Mac.APOE.PTGDS | CD4 | 0.70 |
| CD.B.RPS10.FCER2 | RPS29 | 0.48 | CD.Endth/Ven.LAMP3.LIPG | MADCAM1 | 2.01 | CD.Mac.APOE.PTGDS | GM2A | 0.69 |
| CD.B.RPS10.FCER2 | RPS6 | 0.46 | CD.Endth/Ven.LAMP3.LIPG | PRSS23 | 1.99 | CD.Mac.APOE.PTGDS | RENBP | 0.69 |
| CD.B.RPS10.FCER2 | RPL35 | 0.46 | CD.Endth/Ven.LAMP3.LIPG | CLDN5 | 1.97 | CD.Mac.APOE.PTGDS | TMSB10 | 0.68 |
| CD.B.RPS10.FCER2 | HLA-DRB1 | 0.46 | CD.Endth/Ven.LAMP3.LIPG | PRKCDBP | 1.95 | CD.Mac.APOE.PTGDS | CD209 | 0.68 |
| CD.B.RPS10.FCER2 | RPS27A | 0.45 | CD.Endth/Ven.LAMP3.LIPG | EGFL7 | 1.94 | CD.Mac.APOE.PTGDS | PILRA | 0.68 |
| CD.B.RPS10.FCER2 | RPL21 | 0.44 | CD.Endth/Ven.LAMP3.LIPG | HYAL2 | 1.86 | CD.Mac.APOE.PTGDS | PLTP | 0.68 |
| CD.B.RPS10.FCER2 | RPS13 | 0.43 | CD.Endth/Ven.LAMP3.LIPG | NPDC1 | 1.86 | CD.Mac.APOE.PTGDS | ACP2 | 0.66 |
| CD.B.RPS10.FCER2 | RPL3 | 0.40 | CD.Endth/Ven.LAMP3.LIPG | CD93 | 1.83 | CD.Mac.APOE.PTGDS | NAAA | 0.66 |
| CD.B.RPS10.FCER2 | RPS15A | 0.40 | CD.Endth/Ven.LAMP3.LIPG | TGM2 | 1.80 | CD.Mac.APOE.PTGDS | LILRB5 | 0.65 |
| CD.B.RPS10.FCER2 | RPL34 | 0.39 | CD.Endth/Ven.LAMP3.LIPG | SLCO2A1 | 1.79 | CD.Mac.APOE.PTGDS | KCNMA1 | 0.65 |
| CD.B.RPS10.FCER2 | EEF1B2 | 0.61 | CD.Endth/Ven.LAMP3.LIPG | PTRF | 1.77 | CD.Mac.APOE.PTGDS | VMO1 | 0.64 |
| CD.B.RPS10.FCER2 | RPS9 | 0.41 | CD.Endth/Ven.LAMP3.LIPG | FAM167B | 1.77 | CD.Mac.APOE.PTGDS | CD300A | 0.64 |
| CD.B.RPS10.FCER2 | RPL27A | 0.39 | CD.Endth/Ven.LAMP3.LIPG | SELP | 1.75 | CD.Mac.APOE.PTGDS | SCIMP | 0.63 |
| CD.B.RPS10.FCER2 | HLA-DMA | 0.71 | CD.Endth/Ven.LAMP3.LIPG | PCDH17 | 1.69 | CD.Mac.APOE.PTGDS | LPAR6 | 0.63 |
| CD.B.RPS10.FCER2 | CD19 | 0.80 | CD.Endth/Ven.LAMP3.LIPG | CALCRL | 1.67 | CD.Mac.APOE.PTGDS | B2M | 0.62 |
| CD.B.RPS10.FCER2 | GPR18 | 0.83 | CD.Endth/Ven.LAMP3.LIPG | ECSCR-1 | 1.67 | CD.Mac.APOE.PTGDS | ABHD12 | 0.62 |
| CD.B.RPS29.RPS21 | VPREB3 | 1.49 | CD.Endth/Ven.LAMP3.LIPG | SERPINE1 | 1.65 | CD.Mac.APOE.PTGDS | CLEC4A | 0.61 |
| CD.B.RPS29.RPS21 | MS4A1 | 1.26 | CD.Endth/Ven.LAMP3.LIPG | CLEC14A | 1.57 | CD.Mac.APOE.PTGDS | KCTD12 | 0.60 |
| CD.B.RPS29.RPS21 | CD79A | 1.09 | CD.Endth/Ven.LAMP3.LIPG | APLNR | 1.57 | CD.Mac.APOE.PTGDS | CFD | 0.60 |
| CD.B.RPS29.RPS21 | BANK1 | 1.08 | CD.Endth/Ven.LAMP3.LIPG | IL3RA | 1.51 | CD.Mac.APOE.PTGDS | NAIP | 0.59 |
| CD.B.RPS29.RPS21 | TNFRSF13C | 1.08 | CD.Endth/Ven.LAMP3.LIPG | ARHGAP29 | 1.50 | CD.Mac.APOE.PTGDS | PTGS2 | 0.59 |
| CD.B.RPS29.RPS21 | SMIM14 | 1.01 | CD.Endth/Ven.LAMP3.LIPG | ITGA5 | 1.49 | CD.Mac.APOE.PTGDS | LAIR1 | 0.59 |
| CD.B.RPS29.RPS21 | RP5- | 0.89 | CD.Endth/Ven.LAMP3.LIPG | MCAM | 1.45 | CD.Mac.APOE.PTGDS | CLEC4E | 0.58 |
| | 887A10.1 | | | | | | | |
| CD.B.RPS29.RPS21 | TNFRSF13B | 0.89 | CD.Endth/Ven.LAMP3.LIPG | MCAM | 1.45 | CD.Mac.APOE.PTGDS | CLEC4E | 0.58 |
| CD.B.RPS29.RPS21 | RPS10 | 0.85 | CD.Endth/Ven.LAMP3.LIPG | RAI14 | 1.42 | CD.Mac.APOE.PTGDS | C5AR1 | 0.56 |
| CD.B.RPS29.RPS21 | CD37 | 0.84 | CD.Endth/Ven.LAMP3.LIPG | FLT1 | 1.41 | CD.Mac.APOE.PTGDS | RASSF4 | 0.56 |
| CD.B.RPS29.RPS21 | LY86 | 0.83 | CD.Endth/Ven.LAMP3.LIPG | EMP1 | 1.39 | CD.Mac.APOE.PTGDS | ADAP2 | 0.55 |
| CD.B.RPS29.RPS21 | ARHGAP24 | 0.81 | CD.Endth/Ven.LAMP3.LIPG | FAM198B | 1.39 | CD.Mac.APOE.PTGDS | MSR1 | 0.55 |
| CD.B.RPS29.RPS21 | RPS21 | 0.78 | CD.Endth/Ven.LAMP3.LIPG | SOX18 | 1.38 | CD.Mac.APOE.PTGDS | RAB20 | 0.54 |
| CD.B.RPS29.RPS21 | RPLP2 | 0.77 | CD.Endth/Ven.LAMP3.LIPG | LAMP3 | 1.36 | CD.Mac.APOE.PTGDS | SLC31A2 | 0.54 |
| CD.B.RPS29.RPS21 | CD74 | 0.77 | CD.Endth/Ven.LAMP3.LIPG | RCAN1 | 1.36 | CD.Mac.APOE.PTGDS | LACC1 | 0.54 |
| CD.B.RPS29.RPS21 | RPS8 | 0.76 | CD.Endth/Ven.LAMP3.LIPG | ESAM | 1.34 | CD.Mac.APOE.PTGDS | TFEC | 0.51 |
| CD.B.RPS29.RPS21 | RPL23A | 0.75 | CD.Endth/Ven.LAMP3.LIPG | P2RY6 | 1.28 | CD.Mac.APOE.PTGDS | TIMP2 | 0.51 |
| CD.B.RPS29.RPS21 | RPS29 | 0.74 | CD.Endth/Ven.LAMP3.LIPG | ADGRL4 | 1.26 | CD.Mac.APOE.PTGDS | GNS | 0.50 |
| CD.B.RPS29.RPS21 | RPL36A | 0.72 | CD.Endth/Ven.LAMP3.LIPG | FSTL1 | 1.25 | CD.Mac.APOE.PTGDS | CSF2RA | 0.50 |
| CD.B.RPS29.RPS21 | FCRLA | 0.72 | CD.Endth/Ven.LAMP3.LIPG | CDH5 | 1.24 | CD.Mac.APOE.PTGDS | SLC8A1 | 0.48 |
| CD.B.RPS29.RPS21 | RPS27 | 0.71 | CD.Endth/Ven.LAMP3.LIPG | MMRN2 | 1.18 | CD.Mac.APOE.PTGDS | ABCA1 | 0.48 |
| CD.B.RPS29.RPS21 | GPR183 | 0.71 | CD.Endth/Ven.LAMP3.LIPG | HTRA1 | 1.10 | CD.Mac.APOE.PTGDS | DAPK1 | 0.47 |
| CD.B.RPS29.RPS21 | RPL39 | 0.71 | CD.Endth/Ven.LAMP3.LIPG | RASIP1 | 1.08 | CD.Mac.APOE.PTGDS | CLEC12A | 0.45 |
| CD.B.RPS29.RPS21 | RPL41 | 0.69 | CD.Endth/Ven.LAMP3.LIPG | ZNF385D | 1.07 | CD.Mac.APOE.PTGDS | CD300LF | 0.44 |
| CD.B.RPS29.RPS21 | CD83 | 0.69 | CD.Endth/Ven.LAMP3.LIPG | LIPG | 1.07 | CD.Mac.APOE.PTGDS | PTAFR | 0.43 |
| CD.B.RPS29.RPS21 | RPS18 | 0.68 | CD.Endth/Ven.LAMP3.LIPG | IL33 | 1.02 | CD.Mac.APOE.PTGDS | DMXL2 | 0.41 |
| CD.B.RPS29.RPS21 | RPS23 | 0.67 | CD.Endth/Ven.LAMP3.LIPG | PXDN | 1.02 | CD.Mac.APOE.PTGDS | CMKLR1 | 0.38 |
| CD.B.RPS29.RPS21 | RPL38 | 0.67 | CD.Endth/Ven.LAMP3.LIPG | ROBO4 | 1.01 | CD.Mac.APOE.PTGDS | CD302 | 0.58 |
| CD.B.RPS29.RPS21 | RPS28 | 0.66 | CD.Endth/Ven.LAMP3.LIPG | MEOX1 | 1.01 | CD.Mac.APOE.PTGDS | GNPDA1 | 0.53 |
| CD.B.RPS29.RPS21 | CD79B | 0.65 | CD.Endth/Ven.LAMP3.LIPG | EMCN | 1.00 | CD.Mac.APOE.PTGDS | HLA-DQA2 | 1.14 |
| CD.B.RPS29.RPS21 | RPS25 | 0.64 | CD.Endth/Ven.LAMP3.LIPG | SEMA3F | 1.00 | CD.Mac.APOE.PTGDS | RAB32 | 0.54 |
| CD.B.RPS29.RPS21 | RPL27A | 0.64 | CD.Endth/Ven.LAMP3.LIPG | CYYR1 | 0.99 | CD.Mac.APOE.PTGDS | IFNGR2 | 0.70 |
| CD.B.RPS29.RPS21 | LTB | 0.63 | CD.Endth/Ven.LAMP3.LIPG | CD34 | 0.99 | CD.Mac.APOE.PTGDS | CEBPD | 0.64 |
| CD.B.RPS29.RPS21 | RPL31 | 0.62 | CD.Endth/Ven.LAMP3.LIPG | DPYSL3 | 0.99 | CD.Mac.APOE.PTGDS | SLC15A3 | 0.44 |
| CD.B.RPS29.RPS21 | RPL11 | 0.62 | CD.Endth/Ven.LAMP3.LIPG | ERG | 0.98 | CD.Mac.APOE.PTGDS | RAB31 | 0.49 |
| CD.B.RPS29.RPS21 | EEF1B2 | 0.61 | CD.Endth/Ven.LAMP3.LIPG | RHOJ | 0.97 | CD.Mac.APOE.PTGDS | SIRPA | 0.44 |
| CD.B.RPS29.RPS21 | RPS14 | 0.61 | CD.Endth/Ven.LAMP3.LIPG | JAM2 | 0.96 | CD.Mac.APOE.PTGDS | LITAF | 0.81 |
| CD.B.RPS29.RPS21 | CXCR4 | 0.61 | CD.Endth/Ven.LAMP3.LIPG | KDR | 0.96 | CD.Mac.APOE.PTGDS | LILRB2 | 0.41 |
| CD.B.RPS29.RPS21 | RPS20 | 0.60 | CD.Endth/Ven.LAMP3.LIPG | RAB3C | 0.95 | CD.Mac.APOE.PTGDS | CD163L1 | 0.48 |
| CD.B.RPS29.RPS21 | RPS11 | 0.59 | CD.Endth/Ven.LAMP3.LIPG | TIE1 | 0.93 | CD.Mac.APOE.PTGDS | ATP6AP1 | 0.59 |
| CD.B.RPS29.RPS21 | RPL30 | 0.59 | CD.Endth/Ven.LAMP3.LIPG | TMEM255B | 0.89 | CD.Mac.APOE.PTGDS | SCARB2 | 0.65 |
| CD.B.RPS29.RPS21 | RPL36 | 0.58 | CD.Endth/Ven.LAMP3.LIPG | CXorf36 | 0.87 | CD.Mac.APOE.PTGDS | SDS | 0.58 |
| CD.B.RPS29.RPS21 | RPL13A | 0.58 | CD.Endth/Ven.LAMP3.LIPG | MAP1B | 0.84 | CD.Mac.APOE.PTGDS | LGALS3 | 0.93 |
| CD.B.RPS29.RPS21 | RPL37 | 0.58 | CD.Endth/Ven.LAMP3.LIPG | PLA1A | 0.80 | CD.Mac.APOE.PTGDS | AKR1A1 | 0.90 |
| CD.B.RPS29.RPS21 | RPL19 | 0.58 | CD.Endth/Ven.LAMP3.LIPG | ADAMTS9 | 0.79 | CD.Mac.APOE.PTGDS | NCOA4 | 0.66 |
| CD.B.RPS29.RPS21 | CD52 | 0.57 | CD.Endth/Ven.LAMP3.LIPG | OLFM1 | 0.77 | CD.Mac.APOE.PTGDS | CH17- | 0.46 |
| | | | | | | | 340M24.3 | |
| CD.B.RPS29.RPS21 | RPL26 | 0.57 | CD.Endth/Ven.LAMP3.LIPG | S100A3 | 0.74 | CD.Mac.APOE.PTGDS | STAT1 | 0.90 |
| CD.B.RPS29.RPS21 | RPL23 | 0.57 | CD.Endth/Ven.LAMP3.LIPG | SHANK3 | 0.73 | CD.Mac.APOE.PTGDS | LGALS9 | 0.69 |
| CD.B.RPS29.RPS21 | RPL27 | 0.57 | CD.Endth/Ven.LAMP3.LIPG | FAM110D | 0.72 | CD.Mac.APOE.PTGDS | GPX4 | 0.90 |
| CD.B.RPS29.RPS21 | RPL7A | 0.56 | CD.Endth/Ven.LAMP3.LIPG | MAPK11 | 0.71 | CD.Mac.APOE.PTGDS | SNX10 | 0.81 |
| CD.B.RPS29.RPS21 | RPS5 | 0.56 | CD.Endth/Ven.LAMP3.LIPG | NPR1 | 0.67 | CD.Mac.APOE.PTGDS | DNAJC5B | 0.40 |
| CD.B.RPS29.RPS21 | HLA-DQB1 | 0.55 | CD.Endth/Ven.LAMP3.LIPG | SPNS2 | 0.67 | CD.Mac.APOE.PTGDS | PLEKHO1 | 0.62 |
| CD.B.RPS29.RPS21 | RPS16 | 0.55 | CD.Endth/Ven.LAMP3.LIPG | RAPGEF4 | 0.62 | CD.Mac.APOE.PTGDS | PLBD1 | 0.57 |
| CD.B.RPS29.RPS21 | RPL14 | 0.55 | CD.Endth/Ven.LAMP3.LIPG | DOC2B | 0.42 | CD.Mac.C1QB.FCGR1A | FCGR1A | 1.27 |
| CD.B.RPS29.RPS21 | RPL10A | 0.54 | CD.Endth/Ven.LAMP3.LIPG | GPR4 | 0.58 | CD.Mac.CXCL2.TNF | VSIG4 | 1.38 |
| CD.B.RPS29.RPS21 | FAU | 0.54 | CD.Endth/Ven.LAMP3.LIPG | ACVRL1 | 1.50 | CD.Mac.CXCL2.TNF | KB-1507C5.4 | 0.92 |
| CD.B.RPS29.RPS21 | RPS2 | 0.54 | CD.Endth/Ven.LAMP3.LIPG | MYCT1 | 0.69 | CD.Mac.CXCL3.APOC1 | MS4A4A | 2.26 |
| CD.B.RPS29.RPS21 | RPL5 | 0.53 | CD.Endth/Ven.LAMP3.LIPG | TINAGL1 | 1.27 | CD.Mac.FOLR2.LILRB5 | CCL3 | 3.50 |
| CD.B.RPS29.RPS21 | RPS15A | 0.53 | CD.Endth/Ven.LAMP3.LIPG | GALNT18 | 0.49 | CD.Mac.FOLR2.LILRB5 | CCL3L3 | 3.35 |
| CD.B.RPS29.RPS21 | RPL3 | 0.53 | CD.Endth/Ven.LAMP3.LIPG | MMRN1 | 1.22 | CD.Mac.FOLR2.LILRB5 | CCL4L2 | 3.28 |
| CD.B.RPS29.RPS21 | RPL37A | 0.52 | CD.Endth/Ven.LAMP3.LIPG | JAG2 | 0.72 | CD.Mac.FOLR2.LILRB5 | CXCL8 | 3.27 |
| CD.B.RPS29.RPS21 | RPL8 | 0.52 | CD.Endth/Ven.LAMP3.LIPG | FNDC4 | 0.43 | CD.Mac.FOLR2.LILRB5 | SEPP1 | 3.07 |
| CD.B.RPS29.RPS21 | RPL34 | 0.52 | CD.Endth/Ven.LAMP3.LIPG | LAMA5 | 0.72 | CD.Mac.FOLR2.LILRB5 | CXCL2 | 3.02 |
| CD.B.RPS29.RPS21 | RPSA | 0.52 | CD.Endth/Ven.LAMP3.LIPG | SNAP25 | 0.36 | CD.Mac.FOLR2.LILRB5 | IL1B | 2.97 |
| CD.B.RPS29.RPS21 | RPL9 | 0.52 | CD.Endth/Ven.POSTN.ACKR1 | ACKR1 | 3.60 | CD.Mac.FOLR2.LILRB5 | C1QC | 2.95 |
| CD.B.RPS29.RPS21 | RPL32 | 0.51 | CD.Endth/Ven.POSTN.ACKR1 | MGP | 2.82 | CD.Mac.FOLR2.LILRB5 | CXCL3 | 2.90 |
| CD.B.RPS29.RPS21 | RPL35A | 0.51 | CD.Endth/Ven.POSTN.ACKR1 | CLDN5 | 2.66 | CD.Mac.FOLR2.LILRB5 | C1QA | 2.77 |
| CD.B.RPS29.RPS21 | RPS7 | 0.51 | CD.Endth/Ven.POSTN.ACKR1 | VWF | 2.35 | CD.Mac.FOLR2.LILRB5 | CCL4 | 2.68 |
| CD.B.RPS29.RPS21 | RPS12 | 0.50 | CD.Endth/Ven.POSTN.ACKR1 | TSPAN7 | 2.23 | CD.Mac.FOLR2.LILRB5 | C1QB | 2.68 |
| CD.B.RPS29.RPS21 | RPL12 | 0.50 | CD.Endth/Ven.POSTN.ACKR1 | RAMP3 | 2.15 | CD.Mac.FOLR2.LILRB5 | SLC40A1 | 2.31 |
| CD.B.RPS29.RPS21 | RPL18A | 0.50 | CD.Endth/Ven.POSTN.ACKR1 | RAMP2 | 2.15 | CD.Mac.FOLR2.LILRB5 | IER3 | 2.23 |
| CD.B.RPS29.RPS21 | RPL18 | 0.49 | CD.Endth/Ven.POSTN.ACKR1 | PRSS23 | 2.02 | CD.Mac.FOLR2.LILRB5 | CD14 | 2.20 |
| CD.B.RPS29.RPS21 | RPS6 | 0.49 | CD.Endth/Ven.POSTN.ACKR1 | MADCAM1 | 2.01 | CD.Mac.FOLR2.LILRB5 | PLAUR | 2.20 |
| CD.B.RPS29.RPS21 | HLA-DRA | 0.48 | CD.Endth/Ven.POSTN.ACKR1 | TM4SF1 | 1.98 | CD.Mac.FOLR2.LILRB5 | FOLR2 | 2.18 |
| CD.B.RPS29.RPS21 | EEF1A1 | 0.48 | CD.Endth/Ven.POSTN.ACKR1 | ECSCR-1 | 1.93 | CD.Mac.FOLR2.LILRB5 | DNASE1L3 | 2.18 |
| CD.B.RPS29.RPS21 | RPS3 | 0.47 | CD.Endth/Ven.POSTN.ACKR1 | FAM167B | 1.87 | CD.Mac.FOLR2.LILRB5 | LGMN | 2.18 |
| CD.B.RPS29.RPS21 | RPL29 | 0.47 | CD.Endth/Ven.POSTN.ACKR1 | PLVAP | 1.84 | CD.Mac.FOLR2.LILRB5 | MS4A7 | 2.08 |
| CD.B.RPS29.RPS21 | RPL35 | 0.46 | CD.Endth/Ven.POSTN.ACKR1 | MMRN1 | 1.82 | CD.Mac.FOLR2.LILRB5 | MS4A6A | 2.00 |
| CD.B.RPS29.RPS21 | RPS4X | 0.44 | CD.Endth/Ven.POSTN.ACKR1 | NPDC1 | 1.79 | CD.Mac.FOLR2.LILRB5 | TYROBP | 1.99 |
| CD.B.RPS29.RPS21 | HLA-DPB1 | 0.43 | CD.Endth/Ven.POSTN.ACKR1 | IL33 | 1.76 | CD.Mac.FOLR2.LILRB5 | FCGRT | 1.99 |
| CD.B.RPS29.RPS21 | RPL21 | 0.41 | CD.Endth/Ven.POSTN.ACKR1 | POSTN | 1.73 | CD.Mac.FOLR2.LILRB5 | PTGS2 | 1.96 |
| CD.B.RPS29.RPS21 | RPS27A | 0.41 | CD.Endth/Ven.POSTN.ACKR1 | SNCG | 1.63 | CD.Mac.FOLR2.LILRB5 | AIF1 | 1.92 |
| CD.B.RPS29.RPS21 | RPL13 | 0.41 | CD.Endth/Ven.POSTN.ACKR1 | JAM2 | 1.63 | CD.Mac.FOLR2.LILRB5 | LIPA | 1.90 |
| CD.B.RPS29.RPS21 | RPL28 | 0.41 | CD.Endth/Ven.POSTN.ACKR1 | CLEC14A | 1.58 | CD.Mac.FOLR2.LILRB5 | TNF | 1.85 |
| CD.B.RPS29.RPS21 | RPL15 | 0.41 | CD.Endth/Ven.POSTN.ACKR1 | PCAT19 | 1.41 | CD.Mac.FOLR2.LILRB5 | BCL2A1 | 1.83 |
| CD.B.RPS29.RPS21 | HLA-DQA1 | 0.40 | CD.Endth/Ven.POSTN.ACKR1 | CALCRL | 1.36 | CD.Mac.FOLR2.LILRB5 | FGL2 | 1.82 |
| CD.B.RPS29.RPS21 | RPS15 | 0.39 | CD.Endth/Ven.POSTN.ACKR1 | PLA1A | 1.36 | CD.Mac.FOLR2.LILRB5 | FCER1G | 1.71 |
| CD.B.RPS29.RPS21 | HLA-DPA1 | 0.30 | CD.Endth/Ven.POSTN.ACKR1 | ADGRL4 | 1.29 | CD.Mac.FOLR2.LILRB5 | CPVL | 1.63 |
| CD.B.RPS29.RPS21 | RPS17 | 0.45 | CD.Endth/Ven.POSTN.ACKR1 | SELP | 1.27 | CD.Mac.FOLR2.LILRB5 | CSF1R | 1.62 |
| CD.B.RPS29.RPS21 | RPS13 | 0.42 | CD.Endth/Ven.POSTN.ACKR1 | ZNF385D | 1.26 | CD.Mac.FOLR2.LILRB5 | STAB1 | 1.62 |
| CD.B.RPS29.RPS21 | CD22 | 0.65 | CD.Endth/Ven.POSTN.ACKR1 | CYYR1 | 1.24 | CD.Mac.FOLR2.LILRB5 | MS4A4A | 1.61 |
| CD.B.RPS29.RPS21 | GNB2L1 | 0.46 | CD.Endth/Ven.POSTN.ACKR1 | EMCN | 1.23 | CD.Mac.FOLR2.LILRB5 | CD68 | 1.57 |
| CD.B.RPS29.RPS21 | FCRL2 | 0.73 | CD.Endth/Ven.POSTN.ACKR1 | PALMD | 1.12 | CD.Mac.FOLR2.LILRB5 | VSIG4 | 1.56 |
| CD.B.RPS29.RPS21 | CD19 | 0.69 | CD.Endth/Ven.POSTN.ACKR1 | SELE | 1.12 | CD.Mac.FOLR2.LILRB5 | AXL | 1.55 |
| CD.B.RPS29.RPS21 | RPS26 | 0.46 | CD.Endth/Ven.POSTN.ACKR1 | PCDH17 | 1.11 | CD.Mac.FOLR2.LILRB5 | PLD3 | 1.46 |
| CD.B.UBE2C.KIAA0101 | HMGB2 | 2.39 | CD.Endth/Ven.POSTN.ACKR1 | CXorf36 | 0.99 | CD.Mac.FOLR2.LILRB5 | ICAM1 | 1.45 |
| CD.B.UBE2C.KIAA0101 | STMN1 | 2.35 | CD.Endth/Ven.POSTN.ACKR1 | RAB3C | 0.94 | CD.Mac.FOLR2.LILRB5 | MPEG1 | 1.45 |
| CD.B.UBE2C.KIAA0101 | TUBB | 2.15 | CD.Endth/Ven.POSTN.ACKR1 | TLL1 | 0.93 | CD.Mac.FOLR2.LILRB5 | LILRB5 | 1.41 |
| CD.B.UBE2C.KIAA0101 | KIAA0101 | 2.02 | CD.Endth/Ven.POSTN.ACKR1 | EGFL7 | 1.28 | CD.Mac.FOLR2.LILRB5 | MRC1 | 1.34 |
| CD.B.UBE2C.KIAA0101 | UBE2C | 1.87 | CD.Endth/Ven.POSTN.ACKR1 | SPARCL1 | 3.14 | CD.Mac.FOLR2.LILRB5 | CLEC10A | 1.30 |
| CD.B.UBE2C.KIAA0101 | RGS13 | 1.80 | CD.Endth/Ven.POSTN.ACKR1 | SLCO2A1 | 1.05 | CD.Mac.FOLR2.LILRB5 | RNASE6 | 1.28 |
| CD.B.UBE2C.KIAA0101 | PTMA | 1.22 | CD.Endth/Ven.POSTN.ACKR1 | NNMT | 1.69 | CD.Mac.FOLR2.LILRB5 | MAFB | 1.27 |
| CD.B.UBE2C.KIAA0101 | TCL1A | 1.75 | CD.Endth/Ven.POSTN.ACKR1 | MMRN2 | 0.95 | CD.Mac.FOLR2.LILRB5 | TGFBI | 1.27 |
| CD.B.UBE2C.KIAA0101 | CDK1 | 1.53 | CD.Endth/Ven.POSTN.ACKR1 | IL3RA | 1.11 | CD.Mac.FOLR2.LILRB5 | DAB2 | 1.24 |
| CD.B/DZ.AICDA.PLK1 | UBE2C | 2.93 | CD.Endth/Ven.POSTN.ACKR1 | APLNR | 0.94 | CD.Mac.FOLR2.LILRB5 | CD163 | 1.21 |
| CD.B/DZ.AICDA.PLK1 | TOP2A | 2.79 | CD.Endth/Ven.POSTN.ACKR1 | LDB2 | 1.20 | CD.Mac.FOLR2.LILRB5 | GPR34 | 1.18 |
| CD.B/DZ.AICDA.PLK1 | CENPF | 2.63 | CD.Endth/Ven.POSTN.ACKR1 | RAI14 | 0.95 | CD.Mac.FOLR2.LILRB5 | LPAR6 | 1.16 |
| CD.B/DZ.AICDA.PLK1 | CDK1 | 2.34 | CD.Endth/Ven.POSTN.ACKR1 | LEPR | 0.78 | CD.Mac.FOLR2.LILRB5 | CD163L1 | 1.15 |
| CD.B/DZ.AICDA.PLK1 | CDC20 | 2.26 | CD.Endth/Ven.POSTN.ACKR1 | NRN1 | 0.84 | CD.Mac.FOLR2.LILRB5 | FUCA1 | 1.10 |
| CD.B/DZ.AICDA.PLK1 | CCNB1 | 2.26 | CD.Endth/Ven.POSTN.ACKR1 | CRIP2 | 1.53 | CD.Mac.FOLR2.LILRB5 | MAF | 1.10 |
| CD.B/DZ.AICDA.PLK1 | CCNB2 | 2.21 | CD.Endth/Ven.POSTN.ADGRG6 | ACKR1 | 3.30 | CD.Mac.FOLR2.LILRB5 | CFD | 1.07 |
| CD.B/DZ.AICDA.PLK1 | CDKN3 | 2.15 | CD.Endth/Ven.POSTN.ADGRG6 | CLDN5 | 2.74 | CD.Mac.FOLR2.LILRB5 | GATM | 1.07 |
| CD.B/DZ.AICDA.PLK1 | HMMR | 1.98 | CD.Endth/Ven.POSTN.ADGRG6 | MGP | 2.72 | CD.Mac.FOLR2.LILRB5 | F13A1 | 1.07 |
| CD.B/DZ.AICDA.PLK1 | PLK1 | 1.94 | CD.Endth/Ven.POSTN.ADGRG6 | VWF | 2.68 | CD.Mac.FOLR2.LILRB5 | FPR3 | 1.04 |
| CD.B/DZ.AICDA.PLK1 | BIRC5 | 1.93 | CD.Endth/Ven.POSTN.ADGRG6 | MMRN1 | 2.36 | CD.Mac.FOLR2.LILRB5 | KB-1507C5.4 | 1.04 |
| CD.B/DZ.AICDA.PLK1 | MKI67 | 1.92 | CD.Endth/Ven.POSTN.ADGRG6 | CPE | 2.22 | CD.Mac.FOLR2.LILRB5 | TNFAIP2 | 1.03 |
| CD.B/DZ.AICDA.PLK1 | AICDA | 1.90 | CD.Endth/Ven.POSTN.ADGRG6 | IL33 | 2.14 | CD.Mac.FOLR2.LILRB5 | CD36 | 1.03 |
| CD.B/DZ.AICDA.PLK1 | NUF2 | 1.80 | CD.Endth/Ven.POSTN.ADGRG6 | LIFR | 2.12 | CD.Mac.FOLR2.LILRB5 | IGSF6 | 1.01 |
| CD.B/DZ.AICDA.PLK1 | AURKB | 1.79 | CD.Endth/Ven.POSTN.ADGRG6 | JAM2 | 2.03 | CD.Mac.FOLR2.LILRB5 | CD209 | 1.01 |
| CD.B/DZ.AICDA.PLK1 | ASPM | 1.77 | CD.Endth/Ven.POSTN.ADGRG6 | POSTN | 2.00 | CD.Mac.FOLR2.LILRB5 | SLCO2B1 | 1.01 |
| CD.B/DZ.AICDA.PLK1 | AURKA | 1.77 | CD.Endth/Ven.POSTN.ADGRG6 | ADGRG6 | 1.77 | CD.Mac.FOLR2.LILRB5 | CLEC7A | 0.98 |
| CD.B/DZ.AICDA.PLK1 | CENPE | 1.70 | CD.Endth/Ven.POSTN.ADGRG6 | APLNR | 1.51 | CD.Mac.FOLR2.LILRB5 | C1orf54 | 0.98 |
| CD.B/DZ.AICDA.PLK1 | CENPA | 1.65 | CD.Endth/Ven.POSTN.ADGRG6 | CCL14 | 1.46 | CD.Mac.FOLR2.LILRB5 | IGF1 | 0.94 |
| CD.B/DZ.AICDA.PLK1 | CCNA2 | 1.64 | CD.Endth/Ven.POSTN.ADGRG6 | SELP | 1.44 | CD.Mac.FOLR2.LILRB5 | KCTD12 | 0.93 |
| CD.B/DZ.AICDA.PLK1 | TPX2 | 1.64 | CD.Endth/Ven.POSTN.ADGRG6 | RAB3C | 1.33 | CD.Mac.FOLR2.LILRB5 | FCGRZA | 0.92 |
| CD.B/DZ.AICDA.PLK1 | SGOL2 | 1.63 | CD.Endth/Ven.POSTN.ADGRG6 | CLEC3B | 1.22 | CD.Mac.FOLR2.LILRB5 | C5AR1 | 0.89 |
| CD.B/DZ.AICDA.PLK1 | GTSE1 | 1.58 | CD.Endth/Ven.POSTN.ADGRG6 | ZNF385D | 1.10 | CD.Mac.FOLR2.LILRB5 | C3AR1 | 0.87 |
| CD.B/DZ.AICDA.PLK1 | CDCA8 | 1.51 | CD.Endth/Ven.POSTN.ADGRG6 | TLL1 | 0.98 | CD.Mac.FOLR2.LILRB5 | NLRP3 | 0.87 |
| CD.B/DZ.AICDA.PLK1 | CDCA3 | 1.45 | CD.Endth/Ven.POSTN.ADGRG6 | PCAT19 | 1.80 | CD.Mac.FOLR2.LILRB5 | CXCL12 | 0.81 |
| CD.B/DZ.AICDA.PLK1 | DLGAP5 | 1.43 | CD.Endth/Ven.POSTN.ADGRG6 | TSPAN7 | 2.09 | CD.Mac.FOLR2.LILRB5 | NAIP | 0.81 |
| CD.B/DZ.AICDA.PLK1 | DEPDC1 | 1.42 | CD.Endth/Ven.POSTN.ADGRG6 | ECSCR-1 | 1.67 | CD.Mac.FOLR2.LILRB5 | LACC1 | 0.79 |
| CD.B/DZ.AICDA.PLK1 | PRC1 | 1.37 | CD.Endth/Ven.POSTN.ADGRG6 | CALCRL | 2.20 | CD.Mac.FOLR2.LILRB5 | ADAP2 | 0.78 |
| CD.B/DZ.AICDA.PLK1 | PIF1 | 1.35 | CD.Endth/Ven.POSTN.ADGRG6 | RAMP3 | 2.08 | CD.Mac.FOLR2.LILRB5 | NRP1 | 0.75 |
| CD.B/DZ.AICDA.PLK1 | BCL7A | 1.29 | CD.Endth/Ven.POSTN.ADGRG6 | ADGRL4 | 1.55 | CD.Mac.FOLR2.LILRB5 | MMP10 | 0.75 |
| CD.B/DZ.AICDA.PLK1 | NEK2 | 1.29 | CD.Enteroendocrine.NEUROG3.MLN | CHGA | 3.99 | CD.Mac.FOLR2.LILRB5 | MSR1 | 0.71 |
| CD.B/DZ.AICDA.PLK1 | HJURP | 1.25 | CD.Enteroendocrine.NEUROG3.MLN | HEPACAM2 | 2.36 | CD.Mac.FOLR2.LILRB5 | GFRA2 | 0.69 |
| CD.B/DZ.AICDA.PLK1 | KNSTRN | 1.23 | CD.Enteroendocrine.NEUROG3.MLN | ARC | 2.27 | CD.Mac.FOLR2.LILRB5 | IL1A | 0.67 |
| CD.B/DZ.AICDA.PLK1 | SGOL1 | 1.21 | CD.Enteroendocrine.NEUROG3.MLN | MLN | 2.24 | CD.Mac.FOLR2.LILRB5 | MERTK | 0.49 |
| CD.B/DZ.AICDA.PLK1 | PBK | 1.19 | CD.Enteroendocrine.NEUROG3.MLN | NEUROD1 | 2.07 | CD.Mac.FOLR2.LILRB5 | AOAH | 0.95 |
| CD.B/DZ.AICDA.PLK1 | BUB1 | 1.11 | CD.Enteroendocrine.NEUROG3.MLN | SCGN | 1.97 | CD.Mac.FOLR2.LILRB5 | CTSB | 1.58 |
| CD.B/DZ.AICDA.PLK1 | KIFC1 | 1.11 | CD.Enteroendocrine.NEUROG3.MLN | INSM1 | 1.77 | CD.Mac.FOLR2.LILRB5 | LINC00996 | 0.68 |
| CD.B/DZ.AICDA.PLK1 | NDC80 | 1.11 | CD.Enteroendocrine.NEUROG3.MLN | NKX2-2 | 1.48 | CD.Mac.FOLR2.LILRB5 | HLA-DRB5 | 2.54 |
| CD.B/DZ.AICDA.PLK1 | MND1 | 1.04 | CD.Enteroendocrine.NEUROG3.MLN | NEUROG3 | 1.44 | CD.Mac.FOLR2.LILRB5 | HCK | 0.82 |
| CD.B/DZ.AICDA.PLK1 | KIF2C | 1.02 | CD.Enteroendocrine.NEUROG3.MLN | FEV | 1.39 | CD.Mac.FOLR2.LILRB5 | CYB5D1 | 0.75 |
| CD.B/DZ.AICDA.PLK1 | NUGGC | 1.01 | CD.Enteroendocrine.NEUROG3.MLN | SCG5 | 1.38 | CD.Mac.FOLR2.LILRB5 | RNF130 | 1.00 |
| CD.B/DZ.AICDA.PLK1 | TROAP | 1.01 | CD.Enteroendocrine.NEUROG3.MLN | SMIM6 | 1.37 | CD.Mac.FOLR2.LILRB5 | A2M | 1.03 |
| CD.B/DZ.AICDA.PLK1 | RACGAP1 | 0.98 | CD.Enteroendocrine.NEUROG3.MLN | ISL1 | 1.28 | CD.Mac.FOLR2.LILRB5 | ICAM4 | 0.68 |
| CD.B/DZ.AICDA.PLK1 | CEP55 | 0.98 | CD.Enteroendocrine.NEUROG3.MLN | SCG3 | 1.22 | CD.Mac.FOLR2.LILRB5 | LST1 | 1.07 |
| CD.B/DZ.AICDA.PLK1 | CKAP2L | 0.96 | CD.Enteroendocrine.NEUROG3.MLN | PCSK1 | 1.15 | CD.Mac/cDC1.MKI67.IDO1 | DEFA4 | 1.93 |
| CD.B/DZ.AICDA.PLK1 | KIF23 | 0.94 | CD.Enteroendocrine.NEUROG3.MLN | PAX6 | 1.11 | CD.Mac/DC.CXCL10.CLEC4E | CXCL10 | 3.32 |
| CD.B/DZ.AICDA.PLK1 | NCAPG | 0.91 | CD.Enteroendocrine.NEUROG3.MLN | PAX4 | 1.07 | CD.Mac/DC.CXCL10.CLEC4E | CXCL9 | 2.56 |
| CD.B/DZ.AICDA.PLK1 | CDCA2 | 0.91 | CD.Enteroendocrine.NEUROG3.MLN | NEUROD2 | 1.06 | CD.Mac/DC.CXCL10.CLEC4E | IGSF6 | 2.26 |
| CD.B/DZ.AICDA.PLK1 | GPSM2 | 0.89 | CD.Enteroendocrine.NEUROG3.MLN | RFX6 | 1.03 | CD.Mac/DC.CXCL10.CLEC4E | PLA2G7 | 1.72 |
| CD.B/DZ.AICDA.PLK1 | POLH | 0.89 | CD.Enteroendocrine.NEUROG3.MLN | CADPS | 0.98 | CD.Mac/DC.CXCL10.CLEC4E | CLEC4E | 1.45 |
| CD.B/DZ.AICDA.PLK1 | TTK | 0.88 | CD.Enteroendocrine.NEUROG3.MLN | KRT23 | 0.97 | CD.Mac/DC.CXCL8.NLRP3 | CLEC10A | 2.15 |
| CD.B/DZ.AICDA.PLK1 | KIF11 | 0.86 | CD.Enteroendocrine.NEUROG3.MLN | VWA5B2 | 0.91 | CD.Mac/DC.CXCL8.NLRP3 | PTGS2 | 1.60 |
| CD.B/DZ.AICDA.PLK1 | ARHGAP11A | 0.85 | CD.Enteroendocrine.NEUROG3.MLN | CELF3 | 0.90 | CD.Mac/DC.CXCL8.NLRP3 | VSIG4 | 1.57 |
| CD.B/DZ.AICDA.PLK1 | MYBL1 | 0.80 | CD.Enteroendocrine.NEUROG3.MLN | SLC29A4 | 0.89 | CD.Mac/DC.CXCL8.NLRP3 | OLR1 | 1.22 |
| CD.B/DZ.AICDA.PLK1 | SHCBP1 | 0.80 | CD.Enteroendocrine.NEUROG3.MLN | MEX3A | 0.70 | CD.Mac/DC.CXCL8.NLRP3 | NLRP3 | 1.17 |
| CD.B/DZ.AICDA.PLK1 | BUB1B | 0.76 | CD.Enteroendocrine.NEUROG3.MLN | MIR7-3HG | 0.69 | CD.Mac/DC.CXCL8.NLRP3 | IL1A | 0.68 |
| CD.B/DZ.AICDA.PLK1 | PSRC1 | 0.72 | CD.Enteroendocrine.NEUROG3.MLN | PLCXD3 | 0.68 | CD.Mac/DC.CXCL8.NLRP3 | KB-1507C5.4 | 0.63 |
| CD.B/DZ.AICDA.PLK1 | FAM72D | 0.71 | CD.Enteroendocrine.NEUROG3.MLN | OR51E1 | 0.64 | CD.Mac/DC.CXCL8.NLRP3 | C5AR1 | 1.19 |
| CD.B/DZ.AICDA.PLK1 | KIF4A | 0.68 | CD.Enteroendocrine.NEUROG3.MLN | CLPS | 0.61 | CD.Mcell.CCL23.SPIB | CCL23 | 3.28 |
| CD.B/DZ.AICDA.PLK1 | KIF14 | 0.68 | CD.Enteroendocrine.NEUROG3.MLN | SLC26A7 | 0.55 | CD.Mcell.CCL23.SPIB | CCL25 | 2.70 |
| CD.B/DZ.AICDA.PLK1 | RP11- | 0.68 | CD.Enteroendocrine.NEUROG3.MLN | ST18 | 0.54 | CD.Mcell.CCL23.SPIB | TSPAN8 | 2.70 |
| | 132N15.3 | | | | | | | |
| CD.B/DZ.AICDA.PLK1 | SUGCT | 0.67 | CD.Enteroendocrine.NEUROG3.MLN | TAGLN3 | 0.53 | CD.Mcell.CCL23.SPIB | LGALS4 | 2.56 |
| CD.B/DZ.AICDA.PLK1 | DEPDC1B | 0.67 | CD.Enteroendocrine.NEUROG3.MLN | KIF1A | 0.28 | CD.Mcell.CCL23.SPIB | CCL20 | 2.40 |
| CD.B/DZ.AICDA.PLK1 | KIF20A | 0.66 | CD.Enteroendocrine.NEUROG3.MLN | CRYBA2 | 0.63 | CD.Mcell.CCL23.SPIB | EPCAM | 2.36 |
| CD.B/DZ.AICDA.PLK1 | FAM83D | 0.66 | CD.Enteroendocrine.ONECUT3.CCK | GCG | 6.33 | CD.Mcell.CCL23.SPIB | CLDN3 | 2.22 |
| CD.B/DZ.AICDA.PLK1 | WDR66 | 0.63 | CD.Enteroendocrine.ONECUT3.CCK | PYY | 6.01 | CD.Mcell.CCL23.SPIB | CCL15 | 1.97 |
| CD.B/DZ.AICDA.PLK1 | SPAG5 | 0.60 | CD.Enteroendocrine.ONECUT3.CCK | PCSKIN | 4.34 | CD.Mcell.CCL23.SPIB | CLDN4 | 1.87 |
| CD.B/DZ.AICDA.PLK1 | NMU | 0.54 | CD.Enteroendocrine.ONECUT3.CCK | CHGA | 3.62 | CD.Mcell.CCL23.SPIB | MGST1 | 1.80 |
| CD.B/DZ.AICDA.PLK1 | FAM72C | 0.47 | CD.Enteroendocrine.ONECUT3.CCK | CCK | 3.32 | CD.Mcell.CCL23.SPIB | S100A14 | 1.76 |
| CD.B/DZ.AICDA.PLK1 | SERPINA9 | 0.63 | CD.Enteroendocrine.ONECUT3.CCK | SCG5 | 2.42 | CD.Mcell.CCL23.SPIB | AKR1C3 | 1.56 |
| CD.B/DZ.AICDA.PLK1 | NUSAP1 | 2.23 | CD.Enteroendocrine.ONECUT3.CCK | CPE | 2.18 | CD.Mcell.CCL23.SPIB | FXYD3 | 1.44 |
| CD.B/DZ.AICDA.PLK1 | NCAPH | 0.58 | CD.Enteroendocrine.ONECUT3.CCK | TTR | 2.13 | CD.Mcell.CCL23.SPIB | FABP2 | 1.35 |
| CD.B/DZ.AICDA.PLK1 | PRR11 | 0.86 | CD.Enteroendocrine.ONECUT3.CCK | FEV | 1.92 | CD.Mcell.CCL23.SPIB | CTSE | 1.34 |
| CD.B/DZ.AICDA.PLK1 | MAD2L1 | 1.32 | CD.Enteroendocrine.ONECUT3.CCK | SCGN | 1.77 | CD.Mcell.CCL23.SPIB | KLF5 | 1.25 |
| CD.B/DZ.AICDA.PLK1 | NEIL3 | 0.52 | CD.Enteroendocrine.ONECUT3.CCK | CRYBA2 | 1.77 | CD.Mcell.CCL23.SPIB | GSTA1 | 1.23 |
| CD.B/DZ.AICDA.PLK1 | ARHGEF39 | 0.51 | CD.Enteroendocrine.ONECUT3.CCK | ETV1 | 1.57 | CD.Mcell.CCL23.SPIB | CDX1 | 1.20 |
| CD.B/DZ.AICDA.PLK1 | PARPBP | 0.72 | CD.Enteroendocrine.ONECUT3.CCK | INSM1 | 1.55 | CD.Mcell.CCL23.SPIB | STAP2 | 1.17 |
| CD.B/DZ.AICDA.PLK1 | GCSAM | 1.26 | CD.Enteroendocrine.ONECUT3.CCK | CACNA1A | 1.50 | CD.Mcell.CCL23.SPIB | PPP1R1B | 1.15 |
| CD.B/DZ.AICDA/CDK1 | AICDA | 1.80 | CD.Enteroendocrine.ONECUT3.CCK | DRAIC | 1.44 | CD.Mcell.CCL23.SPIB | FERMT1 | 1.06 |
| CD.B/DZ.AICDA/CDK1 | KIF23 | 1.29 | CD.Enteroendocrine.ONECUT3.CCK | PCSK1 | 1.31 | CD.Mcell.CCL23.SPIB | FAM84A | 1.04 |
| CD.B/DZ.AICDA/CDK1 | FAM72C | 0.73 | CD.Enteroendocrine.ONECUT3.CCK | BEX1 | 1.30 | CD.Mcell.CCL23.SPIB | FAM3D | 1.03 |
| CD.B/DZ.AICDA/CDK1 | RP11- | 0.91 | CD.Enteroendocrine.ONECUT3.CCK | RTN1 | 1.29 | CD.Mcell.CCL23.SPIB | RAB25 | 1.03 |
| | 132N15.3 | | | | | | | |
| CD.B/DZ.HIST1H1B.MKI67 | HIST1H1B | 1.40 | CD.Enteroendocrine.ONECUT3.CCK | MIR7-3HG | 1.15 | CD.Mcell.CCL23.SPIB | SDC4 | 1.02 |
| CD.B/DZ.RGS13.AICDA | TCL1A | 2.25 | CD.Enteroendocrine.ONECUT3.CCK | CADPS | 1.12 | CD.Mcell.CCL23.SPIB | FAM3B | 1.02 |
| CD.B/DZ.RGS13.AICDA | RGS13 | 2.14 | CD.Enteroendocrine.ONECUT3.CCK | ONECUT3 | 1.11 | CD.Mcell.CCL23.SPIB | MIA | 0.99 |
| CD.B/DZ.RGS13.AICDA | CD79B | 2.10 | CD.Enteroendocrine.ONECUT3.CCK | UCN3 | 1.04 | CD.Mcell.CCL23.SPIB | CLDN15 | 0.99 |
| CD.B/DZ.RGS13.AICDA | MARCKSL1 | 2.01 | CD.Enteroendocrine.ONECUT3.CCK | PAX6 | 1.03 | CD.Mcell.CCL23.SPIB | DSC2 | 0.96 |
| CD.B/DZ.RGS13.AICDA | SMIM14 | 1.76 | CD.Enteroendocrine.ONECUT3.CCK | PRPH | 1.02 | CD.Mcell.CCL23.SPIB | GOLM1 | 0.88 |
| CD.B/DZ.RGS13.AICDA | VPREB3 | 1.65 | CD.Enteroendocrine.ONECUT3.CCK | NEUROD1 | 0.95 | CD.Mcell.CCL23.SPIB | HSD3B2 | 0.85 |
| CD.B/DZ.RGS13.AICDA | LIMD2 | 1.57 | CD.Enteroendocrine.ONECUT3.CCK | CHGB | 0.94 | CD.Mcell.CCL23.SPIB | AP1M2 | 0.85 |
| CD.B/DZ.RGS13.AICDA | NEIL1 | 1.52 | CD.Enteroendocrine.ONECUT3.CCK | SCG3 | 0.92 | CD.Mcell.CCL23.SPIB | LAD1 | 0.79 |
| CD.B/DZ.RGS13.AICDA | LRMP | 1.50 | CD.Enteroendocrine.ONECUT3.CCK | RIMBP2 | 0.80 | CD.Mcell.CCL23.SPIB | PPP1R14D | 0.78 |
| CD.B/DZ.RGS13.AICDA | CD79A | 1.46 | CD.Enteroendocrine.ONECUT3.CCK | ISL1 | 0.79 | CD.Mcell.CCL23.SPIB | ANXA13 | 0.78 |
| CD.B/DZ.RGS13.AICDA | ACTG1 | 1.46 | CD.Enteroendocrine.ONECUT3.CCK | VWA5B2 | 0.73 | CD.Mcell.CCL23.SPIB | FAM151A | 0.77 |
| CD.B/DZ.RGS13.AICDA | RP11- | 1.40 | CD.Enteroendocrine.ONECUT3.CCK | PLCXD3 | 0.66 | CD.Mcell.CCL23.SPIB | HEPH | 0.75 |
| | 231C14.7 | | | | | | | |
| CD.B/DZ.RGS13.AICDA | MS4A1 | 1.37 | CD.Enteroendocrine.ONECUT3.CCK | ABCC8 | 0.66 | CD.Mcell.CCL23.SPIB | TNFRSF11A | 0.74 |
| CD.B/DZ.RGS13.AICDA | BASP1 | 1.30 | CD.Enteroendocrine.ONECUT3.CCK | SYT7 | 0.60 | CD.Mcell.CCL23.SPIB | TMEM45B | 0.73 |
| CD.B/DZ.RGS13.AICDA | POUZAF1 | 1.25 | CD.Enteroendocrine.ONECUT3.CCK | DHRS2 | 0.29 | CD.Mcell.CCL23.SPIB | USH1C | 0.73 |
| CD.B/DZ.RGS13.AICDA | BCAS4 | 1.24 | CD.Enteroendocrine.ONECUT3.CCK | TM4SF4 | 1.23 | CD.Mcell.CCL23.SPIB | LPL | 0.71 |
| CD.B/DZ.RGS13.AICDA | SUGCT | 1.24 | CD.Enteroendocrine.TFPI2.TPH1 | CHGA | 5.67 | CD.Mcell.CCL23.SPIB | SDR16C5 | 0.67 |
| CD.B/DZ.RGS13.AICDA | BIK | 1.22 | CD.Enteroendocrine.TFPI2.TPH1 | PCSKIN | 3.67 | CD.Mcell.CCL23.SPIB | CBLC | 0.65 |
| CD.B/DZ.RGS13.AICDA | TNFRSF13C | 1.21 | CD.Enteroendocrine.TFPI2.TPH1 | CHGB | 3.57 | CD.Mcell.CCL23.SPIB | SLC44A3 | 0.64 |
| CD.B/DZ.RGS13.AICDA | IRF8 | 1.21 | CD.Enteroendocrine.TFPI2.TPH1 | TPH1 | 3.43 | CD.Mcell.CCL23.SPIB | MAL2 | 0.63 |
| CD.B/DZ.RGS13.AICDA | POU2F2 | 1.20 | CD.Enteroendocrine.TFPI2.TPH1 | CES1 | 2.47 | CD.Mcell.CCL23.SPIB | CXADR | 0.63 |
| CD.B/DZ.RGS13.AICDA | SERPINA9 | 1.19 | CD.Enteroendocrine.TFPI2.TPH1 | TAC1 | 2.37 | CD.Mcell.CCL23.SPIB | GABRP | 0.60 |
| CD.B/DZ.RGS13.AICDA | RFTN1 | 1.16 | CD.Enteroendocrine.TFPI2.TPH1 | FEV | 2.20 | CD.Mcell.CCL23.SPIB | NOXO1 | 0.60 |
| CD.B/DZ.RGS13.AICDA | BCL7A | 1.14 | CD.Enteroendocrine.TFPI2.TPH1 | SCGN | 1.99 | CD.Mcell.CCL23.SPIB | AQP1 | 0.58 |
| CD.B/DZ.RGS13.AICDA | SPIB | 1.13 | CD.Enteroendocrine.TFPI2.TPH1 | PCSK1 | 1.97 | CD.Mcell.CCL23.SPIB | ABO | 0.58 |
| CD.B/DZ.RGS13.AICDA | RGS16 | 1.13 | CD.Enteroendocrine.TFPI2.TPH1 | CRYBA2 | 1.92 | CD.Mcell.CCL23.SPIB | LECT1 | 0.52 |
| CD.B/DZ.RGS13.AICDA | CD22 | 1.12 | CD.Enteroendocrine.TFPI2.TPH1 | CPE | 1.79 | CD.Mcell.CCL23.SPIB | SOX8 | 0.52 |
| CD.B/DZ.RGS13.AICDA | AC023590.1 | 1.07 | CD.Enteroendocrine.TFPI2.TPH1 | SCG5 | 1.79 | CD.Mcell.CCL23.SPIB | CLRN3 | 0.51 |
| CD.B/DZ.RGS13.AICDA | FCRLA | 1.04 | CD.Enteroendocrine.TFPI2.TPH1 | ARC | 1.78 | CD.Mcell.CCL23.SPIB | ANXA3 | 0.46 |
| CD.B/DZ.RGS13.AICDA | AICDA | 1.00 | CD.Enteroendocrine.TFPI2.TPH1 | SLC38A11 | 1.62 | CD.Mcell.CCL23.SPIB | TINAG | 0.43 |
| CD.B/DZ.RGS13.AICDA | DCAF12 | 0.93 | CD.Enteroendocrine.TFPI2.TPH1 | SYT13 | 1.61 | CD.Mcell.CCL23.SPIB | GJB3 | 0.43 |
| CD.B/DZ.RGS13.AICDA | WDR66 | 0.72 | CD.Enteroendocrine.TFPI2.TPH1 | RAB3C | 1.44 | CD.Mcell.CCL23.SPIB | IL17RB | 0.40 |
| CD.B/DZ.RGS13.AICDA | TCEA1 | 1.41 | CD.Enteroendocrine.TFPI2.TPH1 | BEX1 | 1.43 | CD.Mcell.CCL23.SPIB | NTRK2 | 0.38 |
| CD.B/DZ.RGS13.AICDA | CD37 | 1.40 | CD.Enteroendocrine.TFPI2.TPH1 | NEUROD1 | 1.34 | CD.Mcell.CCL23.SPIB | CEL | 0.30 |
| CD.B/DZ.RGS13.AICDA | GGA2 | 1.06 | CD.Enteroendocrine.TFPI2.TPH1 | RAB3B | 1.26 | CD.Mcell.CCL23.SPIB | UGT2B17 | 1.68 |
| CD.B/DZ.RGS13.AICDA | SERF2 | 1.08 | CD.Enteroendocrine.TFPI2.TPH1 | TM4SF4 | 1.26 | CD.Mcell.CCL23.SPIB | PRAP1 | 1.17 |
| CD.B/DZ.RGS13.AICDA | VNN2 | 0.82 | CD.Enteroendocrine.TFPI2.TPH1 | TTR | 1.25 | CD.Mcell.CCL23.SPIB | PLPP2 | 0.72 |
| CD.B/DZ.RGS13.AICDA | HRK | 0.61 | CD.Enteroendocrine.TFPI2.TPH1 | CACNA1A | 1.24 | CD.Mcell.CCL23.SPIB | PERP | 1.44 |
| CD.B/DZ.RGS13.AICDA | RP11- | 0.56 | CD.Enteroendocrine.TFPI2.TPH1 | PSCA | 1.07 | CD.Mcell.CCL23.SPIB | PRSS8 | 0.74 |
| | 132N15.3 | | | | | | | |
| CD.B/DZ.RGS13.AICDA | P2RX5 | 0.85 | CD.Enteroendocrine.TFPI2.TPH1 | NKX2-2 | 1.05 | CD.Mcell.CCL23.SPIB | AGR3 | 1.20 |
| CD.B/DZ.RGS13.AICDA | HMCES | 1.13 | CD.Enteroendocrine.TFPI2.TPH1 | INSM1 | 0.99 | CD.Mcell.CCL23.SPIB | HOXB9 | 0.41 |
| CD.B/GC.CENPE.TOP2A | MKI67 | 2.29 | CD.Enteroendocrine.TFPI2.TPH1 | RPRML | 0.96 | CD.Mcell.CCL23.SPIB | CLDN7 | 1.40 |
| CD.B/GC.CENPE.TOP2A | TOP2A | 2.29 | CD.Enteroendocrine.TFPI2.TPH1 | SLC18A1 | 0.94 | CD.Mcell.CCL23.SPIB | CSRP2 | 0.55 |
| CD.B/GC.CENPE.TOP2A | CENPF | 2.15 | CD.Enteroendocrine.TFPI2.TPH1 | OR51E1 | 0.93 | CD.Mcell.CCL23.SPIB | GPX2 | 1.89 |
| CD.B/GC.CENPE.TOP2A | NUSAP1 | 2.13 | CD.Enteroendocrine.TFPI2.TPH1 | SCG2 | 0.89 | CD.Mcell.CCL23.SPIB | SPINT1 | 0.87 |
| CD.B/GC.CENPE.TOP2A | UBE2C | 2.06 | CD.Enteroendocrine.TFPI2.TPH1 | SCG3 | 0.85 | CD.Mcell.CCL23.SPIB | PTPRF | 0.67 |
| CD.B/GC.CENPE.TOP2A | RGS13 | 1.91 | CD.Enteroendocrine.TFPI2.TPH1 | ADGRG4 | 0.82 | CD.Mcell.CCL23.SPIB | HOXB8 | 0.29 |
| CD.B/GC.CENPE.TOP2A | CDK1 | 1.83 | CD.Enteroendocrine.TFPI2.TPH1 | ST18 | 0.76 | CD.Mcell.CCL23.SPIB | SFN | 0.75 |
| CD.B/GC.CENPE.TOP2A | KIAA0101 | 1.73 | CD.Enteroendocrine.TFPI2.TPH1 | VWA5B2 | 0.72 | CD.Mcell.CCL23.SPIB | KRT19 | 1.75 |
| CD.B/GC.CENPE.TOP2A | NEIL1 | 1.68 | CD.Enteroendocrine.TFPI2.TPH1 | MIR7-3HG | 0.68 | CD.Mcell.CCL23.SPIB | CDH17 | 0.95 |
| CD.B/GC.CENPE.TOP2A | AURKB | 1.52 | CD.Enteroendocrine.TFPI2.TPH1 | RIMBP2 | 0.62 | CD.Mcell.CCL23.SPIB | C6orf141 | 0.52 |
| CD.B/GC.CENPE.TOP2A | ASPM | 1.51 | CD.Enteroendocrine.TFPI2.TPH1 | RFX6 | 0.56 | CD.Mcell.CCL23.SPIB | IFT172 | 0.74 |
| CD.B/GC.CENPE.TOP2A | CENPE | 1.51 | CD.Enteroendocrine.TFPI2.TPH1 | C2orf54 | 0.53 | CD.Mcell.CCL23.SPIB | TRABD2A | 0.69 |
| CD.B/GC.CENPE.TOP2A | CDKN3 | 1.48 | CD.Enteroendocrine.TFPI2.TPH1 | PAX6 | 0.50 | CD.Mcell.CSRP2.SPIB | GJB3 | 0.57 |
| CD.B/GC.CENPE.TOP2A | HMMR | 1.47 | CD.Enteroendocrine.TFPI2.TPH1 | SNAP25 | 0.49 | CD.Mcell.CSRP2.SPIB | RHOV | 0.47 |
| CD.B/GC.CENPE.TOP2A | AICDA | 1.47 | CD.Enteroendocrine.TFPI2.TPH1 | CELF3 | 0.26 | CD.Mito.MT-CO2.MT-ND2 | C1QA | 1.83 |
| CD.B/GC.CENPE.TOP2A | CCNB2 | 1.44 | CD.Enteroendocrine.TFPI2.TPH1 | KCNJ6 | 0.52 | CD.Mito.MT-CO2.MT-ND2 | PSAP | 1.69 |
| CD.B/GC.CENPE.TOP2A | TPX2 | 1.43 | CD.Enteroendocrine.TFPI2.TPH1 | SLC29A4 | 0.71 | CD.Mito.MT-CO2.MT-ND2 | IL1B | 1.63 |
| CD.B/GC.CENPE.TOP2A | TYMS | 1.40 | CD.Enteroendocrine.TFPI2.TPH1 | MS4A8 | 1.46 | CD.Mito.MT-CO2.MT-ND2 | FGL2 | 1.57 |
| CD.B/GC.CENPE.TOP2A | RRM2 | 1.39 | CD.Enteroendocrine.TPH1.CHGA | CHGA | 3.96 | CD.Mito.MT-CO2.MT-ND2 | CXCL8 | 1.51 |
| CD.B/GC.CENPE.TOP2A | MXD3 | 1.39 | CD.Enteroendocrine.TPH1.CHGA | PCSKIN | 2.93 | CD.Mito.MT-CO2.MT-ND2 | SAT1 | 1.46 |
| CD.B/GC.CENPE.TOP2A | BIRC5 | 1.38 | CD.Enteroendocrine.TPH1.CHGA | CHGB | 2.46 | CD.Mito.MT-CO2.MT-ND2 | MT-CO1 | 1.46 |
| CD.B/GC.CENPE.TOP2A | TK1 | 1.37 | CD.Enteroendocrine.TPH1.CHGA | TPH1 | 2.32 | CD.Mito.MT-CO2.MT-ND2 | AIF1 | 1.42 |
| CD.B/GC.CENPE.TOP2A | GCSAM | 1.37 | CD.Enteroendocrine.TPH1.CHGA | NKX2-2 | 0.84 | CD.Mito.MT-CO2.MT-ND2 | HLA-DRB1 | 1.39 |
| CD.B/GC.CENPE.TOP2A | BCL7A | 1.36 | CD.Enteroendocrine.TPH1.CHGA | VWA5B2 | 0.66 | CD.Mito.MT-CO2.MT-ND2 | MT-ATP6 | 1.38 |
| CD.B/GC.CENPE.TOP2A | SGOL2 | 1.36 | CD.Enteroendocrine.TPH1.CHGA | RPRML | 0.62 | CD.Mito.MT-CO2.MT-ND2 | MT-CO2 | 1.37 |
| CD.B/GC.CENPE.TOP2A | CDC20 | 1.35 | CD.EpithStem.LINC00176.RPS4Y1 | RNF186 | 0.64 | CD.Mito.MT-CO2.MT-ND2 | TYROBP | 1.36 |
| CD.B/GC.CENPE.TOP2A | CENPA | 1.29 | CD.EpithStem.LINC00176.RPS4Y1 | LGR5 | 0.58 | CD.Mito.MT-CO2.MT-ND2 | CST3 | 1.36 |
| CD.B/GC.CENPE.TOP2A | NUF2 | 1.26 | CD.EpithStem.LINC00176.RPS4Y1 | KIF12 | 0.48 | CD.Mito.MT-CO2.MT-ND2 | MT-ND3 | 1.35 |
| CD.B/GC.CENPE.TOP2A | GTSE1 | 1.26 | CD.EpithStem.LINC00176.RPS4Y1 | KIAA1324 | 0.86 | CD.Mito.MT-CO2.MT-ND2 | C1QB | 1.34 |
| CD.B/GC.CENPE.TOP2A | CCNA2 | 1.26 | CD.Fibro.ADAMDEC1.C7 | ADAMDEC1 | 4.69 | CD.Mito.MT-CO2.MT-ND2 | MT-CO3 | 1.34 |
| CD.B/GC.CENPE.TOP2A | CDCA3 | 1.26 | CD.Fibro.ADAMDEC1.C7 | LUM | 3.77 | CD.Mito.MT-CO2.MT-ND2 | C1QC | 1.32 |
| CD.B/GC.CENPE.TOP2A | CCNB1 | 1.21 | CD.Fibro.ADAMDEC1.C7 | CFD | 3.46 | CD.Mito.MT-CO2.MT-ND2 | MT-ND4 | 1.30 |
| CD.B/GC.CENPE.TOPZA | NDC80 | 1.21 | CD.Fibro.ADAMDEC1.C7 | MFAP4 | 3.43 | CD.Mito.MT-CO2.MT-ND2 | MT-ND5 | 1.29 |
| CD.B/GC.CENPE.TOP2A | PLK1 | 1.16 | CD.Fibro.ADAMDEC1.C7 | DCN | 3.17 | CD.Mito.MT-CO2.MT-ND2 | MT-ND2 | 1.26 |
| CD.B/GC.CENPE.TOP2A | DLGAP5 | 1.16 | CD.Fibro.ADAMDEC1.C7 | FBLN1 | 2.91 | CD.Mito.MT-CO2.MT-ND2 | FTL | 1.26 |
| CD.B/GC.CENPE.TOP2A | SGOL1 | 1.10 | CD.Fibro.ADAMDEC1.C7 | CCL13 | 2.84 | CD.Mito.MT-CO2.MT-ND2 | HLA-DPB1 | 1.25 |
| CD.B/GC.CENPE.TOP2A | ZWINT | 1.09 | CD.Fibro.ADAMDEC1.C7 | CXCL14 | 2.83 | CD.Mito.MT-CO2.MT-ND2 | MT-ND1 | 1.22 |
| CD.B/GC.CENPE.TOP2A | SPC25 | 1.06 | CD.Fibro.ADAMDEC1.C7 | COL1A2 | 2.82 | CD.Mito.MT-CO2.MT-ND2 | LYZ | 1.21 |
| CD.B/GC.CENPE.TOP2A | CDCA8 | 1.06 | CD.Fibro.ADAMDEC1.C7 | COL3A1 | 2.74 | CD.Mito.MT-CO2.MT-ND2 | MT-CYB | 1.16 |
| CD.B/GC.CENPE.TOP2A | PIF1 | 1.06 | CD.Fibro.ADAMDEC1.C7 | CFH | 2.71 | CD.Mito.MT-CO2.MT-ND2 | HLA-DRA | 1.09 |
| CD.B/GC.CENPE.TOP2A | NCAPG | 1.04 | CD.Fibro.ADAMDEC1.C7 | CCL11 | 2.66 | CD.Mono.CXCL10.TNF | CCL3 | 3.93 |
| CD.B/GC.CENPE.TOP2A | AURKA | 1.04 | CD.Fibro.ADAMDEC1.C7 | C15 | 2.66 | CD.Mono.CXCL10.TNF | CCL3L3 | 3.56 |
| CD.B/GC.CENPE.TOP2A | SPC24 | 0.98 | CD.Fibro.ADAMDEC1.C7 | CXCL12 | 2.61 | CD.Mono.CXCL10.TNF | IL1B | 3.24 |
| CD.B/GC.CENPE.TOP2A | MYBL1 | 0.97 | CD.Fibro.ADAMDEC1.C7 | C1R | 2.47 | CD.Mono.CXCL10.TNF | CXCL10 | 3.20 |
| CD.B/GC.CENPE.TOPZA | CASC5 | 0.96 | CD.Fibro.ADAMDEC1.C7 | CCL8 | 2.38 | CD.Mono.CXCL10.TNF | CCL4 | 3.06 |
| CD.B/GC.CENPE.TOP2A | HJURP | 0.95 | CD.Fibro.ADAMDEC1.C7 | APOE | 2.35 | CD.Mono.CXCL10.TNF | CXCL8 | 2.98 |
| CD.B/GC.CENPE.TOP2A | KIF11 | 0.95 | CD.Fibro.ADAMDEC1.C7 | CCL2 | 2.25 | CD.Mono.CXCL10.TNF | CCL4L2 | 2.94 |
| CD.B/GC.CENPE.TOP2A | SERPINA9 | 0.92 | CD.Fibro.ADAMDEC1.C7 | COL6A2 | 2.20 | CD.Mono.CXCL10.TNF | CXCL2 | 2.79 |
| CD.B/GC.CENPE.TOP2A | PBK | 0.91 | CD.Fibro.ADAMDEC1.C7 | GPX3 | 2.19 | CD.Mono.CXCL10.TNF | SOD2 | 2.71 |
| CD.B/GC.CENPE.TOP2A | PRC1 | 0.90 | CD.Fibro.ADAMDEC1.C7 | RARRES2 | 2.19 | CD.Mono.CXCL10.TNF | TNF | 2.60 |
| CD.B/GC.CENPE.TOP2A | KIFC1 | 0.90 | CD.Fibro.ADAMDEC1.C7 | IGFBP7 | 2.18 | CD.Mono.CXCL10.TNF | CXCL3 | 2.59 |
| CD.B/GC.CENPE.TOP2A | HIST1H1B | 0.85 | CD.Fibro.ADAMDEC1.C7 | COL1A1 | 2.17 | CD.Mono.CXCL10.TNF | BCL2A1 | 2.36 |
| CD.B/GC.CENPE.TOP2A | MND1 | 0.82 | CD.Fibro.ADAMDEC1.C7 | GSN | 2.13 | CD.Mono.CXCL10.TNF | GOS2 | 2.26 |
| CD.B/GC.CENPE.TOP2A | NUGGC | 0.82 | CD.Fibro.ADAMDEC1.C7 | TCF21 | 2.13 | CD.Mono.CXCL10.TNF | CXCL9 | 2.21 |
| CD.B/GC.CENPE.TOP2A | CKAP2L | 0.80 | CD.Fibro.ADAMDEC1.C7 | CALD1 | 2.12 | CD.Mono.CXCL10.TNF | PLAUR | 2.20 |
| CD.B/GC.CENPE.TOP2A | C21orf58 | 0.78 | CD.Fibro.ADAMDEC1.C7 | ABCA8 | 2.11 | CD.Mono.CXCL10.TNF | S100A8 | 2.19 |
| CD.B/GC.CENPE.TOP2A | BUB1 | 0.77 | CD.Fibro.ADAMDEC1.C7 | PLTP | 2.07 | CD.Mono.CXCL10.TNF | IER3 | 2.18 |
| CD.B/GC.CENPE.TOP2A | CDCA2 | 0.77 | CD.Fibro.ADAMDEC1.C7 | C7 | 2.06 | CD.Mono.CXCL10.TNF | S100A9 | 2.18 |
| CD.B/GC.CENPE.TOP2A | ASF1B | 0.77 | CD.Fibro.ADAMDEC1.C7 | HAPLN1 | 2.05 | CD.Mono.CXCL10.TNF | AIF1 | 2.16 |
| CD.B/GC.CENPE.TOP2A | DEPDC1 | 0.76 | CD.Fibro.ADAMDEC1.C7 | TMEM176B | 2.05 | CD.Mono.CXCL10.TNF | PLEK | 2.08 |
| CD.B/GC.CENPE.TOP2A | CEP55 | 0.76 | CD.Fibro.ADAMDEC1.C7 | EDIL3 | 2.01 | CD.Mono.CXCL10.TNF | FCER1G | 2.04 |
| CD.B/GC.CENPE.TOP2A | KIF2C | 0.74 | CD.Fibro.ADAMDEC1.C7 | SPARC | 2.00 | CD.Mono.CXCL10.TNF | TYROBP | 2.02 |
| CD.B/GC.CENPE.TOPZA | KIF23 | 0.71 | CD.Fibro.ADAMDEC1.C7 | IFITM3 | 1.98 | CD.Mono.CXCL10.TNF | IL1RN | 1.92 |
| CD.B/GC.CENPE.TOP2A | ARHGAP11A | 0.70 | CD.Fibro.ADAMDEC1.C7 | A2M | 1.98 | CD.Mono.CXCL10.TNF | PTGS2 | 1.83 |
| CD.B/GC.CENPE.TOP2A | BUB1B | 0.68 | CD.Fibro.ADAMDEC1.C7 | PLPP3 | 1.95 | CD.Mono.CXCL10.TNF | LST1 | 1.75 |
| CD.B/GC.CENPE.TOP2A | CCNF | 0.65 | CD.Fibro.ADAMDEC1.C7 | LTBP4 | 1.93 | CD.Mono.CXCL10.TNF | FCN1 | 1.66 |
| CD.B/GC.CENPE.TOP2A | RP11- | 0.63 | CD.Fibro.ADAMDEC1.C7 | TM4SF1 | 1.92 | CD.Mono.CXCL10.TNF | TNFAIP6 | 1.63 |
| | 132N15.3 | | | | | | | |
| CD.B/GC.CENPE.TOP2A | KIF14 | 0.60 | CD.Fibro.ADAMDEC1.C7 | CYGB | 1.92 | CD.Mono.CXCL10.TNF | TNFAIP2 | 1.61 |
| CD.B/GC.CENPE.TOP2A | DEPDC1B | 0.58 | CD.Fibro.ADAMDEC1.C7 | PPP1R14A | 1.87 | CD.Mono.CXCL10.TNF | FGL2 | 1.58 |
| CD.B/GC.CENPE.TOP2A | KIF4A | 0.57 | CD.Fibro.ADAMDEC1.C7 | MMP2 | 1.86 | CD.Mono.CXCL10.TNF | CYBB | 1.58 |
| CD.B/GC.CENPE.TOP2A | FOXM1 | 0.53 | CD.Fibro.ADAMDEC1.C7 | PMP22 | 1.85 | CD.Mono.CXCL10.TNF | CD14 | 1.53 |
| CD.B/GC.CENPE.TOP2A | FAM72D | 0.40 | CD.Fibro.ADAMDEC1.C7 | CTSC | 1.85 | CD.Mono.CXCL10.TNF | MNDA | 1.48 |
| CD.B/GC.CENPE.TOP2A | KIF15 | 0.58 | CD.Fibro.ADAMDEC1.C7 | ADH1B | 1.84 | CD.Mono.CXCL10.TNF | MS4A7 | 1.41 |
| CD.B/GC.CENPE.TOPZA | ARHGAP11B | 0.86 | CD.Fibro.ADAMDEC1.C7 | PTGDS | 1.83 | CD.Mono.CXCL10.TNF | SERPINA1 | 1.39 |
| CD.B/GC.CENPE.TOP2A | TCL1A | 2.36 | CD.Fibro.ADAMDEC1.C7 | ANGPTL1 | 1.82 | CD.Mono.CXCL10.TNF | CCRL2 | 1.27 |
| CD.B/GC.CENPE.TOP2A | NMU | 0.50 | CD.Fibro.ADAMDEC1.C7 | NBL1 | 1.80 | CD.Mono.CXCL10.TNF | FCGR3A | 1.23 |
| CD.B/GC.CENPE.TOP2A | SUGCT | 0.79 | CD.Fibro.ADAMDEC1.C7 | RBP1 | 1.79 | CD.Mono.CXCL10.TNF | FCGR1A | 1.22 |
| CD.B/GC.CENPE.TOP2A | HIST1H1D | 1.14 | CD.Fibro.ADAMDEC1.C7 | PLPP1 | 1.79 | CD.Mono.CXCL10.TNF | NLRP3 | 1.17 |
| CD.B/GC.CENPE.TOP2A | MYBL2 | 0.82 | CD.Fibro.ADAMDEC1.C7 | GGT5 | 1.78 | CD.Mono.CXCL10.TNF | CLEC4E | 1.04 |
| CD.B/GC.CENPE.TOP2A | PSRC1 | 0.37 | CD.Fibro.ADAMDEC1.C7 | QSOX1 | 1.74 | CD.Mono.CXCL10.TNF | ANKRD22 | 1.00 |
| CD.B/GC.CENPE.TOP2A | HIST1H1A | 0.30 | CD.Fibro.ADAMDEC1.C7 | VCAM1 | 1.72 | CD.Mono.CXCL10.TNF | KCNJ2 | 0.96 |
| CD.B/GC.CENPE.TOP2A | DCAF12 | 1.07 | CD.Fibro.ADAMDEC1.C7 | ASPN | 1.72 | CD.Mono.CXCL10.TNF | KB-1507C5.4 | 0.90 |
| CD.B/GC.CENPE.TOP2A | CDT1 | 0.90 | CD.Fibro.ADAMDEC1.C7 | MEG3 | 1.69 | CD.Mono.CXCL10.TNF | IL1A | 0.76 |
| CD.B/GC.CENPE.TOP2A | MME | 0.67 | CD.Fibro.ADAMDEC1.C7 | SERPINF1 | 1.69 | CD.Mono.CXCL10.TNF | AQP9 | 0.76 |
| CD.B/GC.CENPE.TOP2A | CDKN2D | 1.11 | CD.Fibro.ADAMDEC1.C7 | TMEM176A | 1.68 | CD.Mono.CXCL10.TNF | IRG1 | 0.63 |
| CD.B/GC.CENPE.TOP2A | SAPCD2 | 0.49 | CD.Fibro.ADAMDEC1.C7 | SPARCL1 | 1.68 | CD.Mono.CXCL10.TNF | GPR84 | 0.58 |
| CD.B/GC.CENPE.TOPZA | CCDC171 | 0.55 | CD.Fibro.ADAMDEC1.C7 | FABP5 | 1.67 | CD.Mono.CXCL10.TNF | IGSF6 | 1.22 |
| CD.B/GC.CENPE.TOP2A | EML6 | 0.52 | CD.Fibro.ADAMDEC1.C7 | SPON2 | 1.66 | CD.Mono.CXCL10.TNF | FAM26F | 1.87 |
| CD.B/GC.CENPE.TOP2A | BACH2 | 0.79 | CD.Fibro.ADAMDEC1.C7 | SEPP1 | 1.66 | CD.Mono.CXCL10.TNF | PILRA | 0.90 |
| CD.B/GC.CENPE.TOP2A | SHCBP1 | 0.68 | CD.Fibro.ADAMDEC1.C7 | EMILIN1 | 1.66 | CD.Mono.CXCL10.TNF | NINJ1 | 1.52 |
| CD.B/GC.CENPE.TOP2A | PTTG1 | 1.90 | CD.Fibro.ADAMDEC1.C7 | PDLIM1 | 1.65 | CD.Mono.CXCL10.TNF | RP11- | 2.07 |
| | | | | | | | 1143G9.4 | |
| CD.B/GC.HIST1H1A.RRM2 | TK1 | 2.03 | CD.Fibro.ADAMDEC1.C7 | LGALS1 | 1.63 | CD.Mono.CXCL10.TNF | LYZ | 2.04 |
| CD.B/GC.HIST1H1A.RRM2 | TYMS | 1.87 | CD.Fibro.ADAMDEC1.C7 | PTN | 1.62 | CD.Mono.CXCL3.FCN1 | CXCL8 | 2.97 |
| CD.B/GC.HIST1H1A.RRM2 | RRM2 | 1.83 | CD.Fibro.ADAMDEC1.C7 | TFPI | 1.62 | CD.Mono.CXCL3.FCN1 | IL1B | 2.84 |
| CD.B/GC.HIST1H1A.RRM2 | DHFR | 1.68 | CD.Fibro.ADAMDEC1.C7 | CXCL6 | 1.60 | CD.Mono.CXCL3.FCN1 | CCL3 | 2.81 |
| CD.B/GC.HIST1H1A.RRM2 | MKI67 | 1.57 | CD.Fibro.ADAMDEC1.C7 | IGFBP3 | 1.59 | CD.Mono.CXCL3.FCN1 | CXCL2 | 2.67 |
| CD.B/GC.HIST1H1A.RRM2 | RMI2 | 1.41 | CD.Fibro.ADAMDEC1.C7 | SERPING1 | 1.59 | CD.Mono.CXCL3.FCN1 | CXCL3 | 2.57 |
| CD.B/GC.HIST1H1A.RRM2 | CDT1 | 1.38 | CD.Fibro.ADAMDEC1.C7 | PROCR | 1.58 | CD.Mono.CXCL3.FCN1 | CCL3L3 | 2.20 |
| CD.B/GC.HIST1H1A.RRM2 | CDK1 | 1.25 | CD.Fibro.ADAMDEC1.C7 | VIM | 1.57 | CD.Mono.CXCL3.FCN1 | PLAUR | 1.99 |
| CD.B/GC.HIST1H1A.RRM2 | ASF1B | 1.21 | CD.Fibro.ADAMDEC1.C7 | S100A13 | 1.56 | CD.Mono.CXCL3.FCN1 | LST1 | 1.84 |
| CD.B/GC.HIST1H1A.RRM2 | CLSPN | 1.19 | CD.Fibro.ADAMDEC1.C7 | SNAI2 | 1.55 | CD.Mono.CXCL3.FCN1 | FGL2 | 1.73 |
| CD.B/GC.HIST1H1A.RRM2 | SUGCT | 1.06 | CD.Fibro.ADAMDEC1.C7 | OLFML3 | 1.55 | CD.Mono.CXCL3.FCN1 | EREG | 1.70 |
| CD.B/GC.HIST1H1A.RRM2 | FAM111B | 1.04 | CD.Fibro.ADAMDEC1.C7 | NUPR1 | 1.54 | CD.Mono.CXCL3.FCN1 | PTGS2 | 1.67 |
| CD.B/GC.HIST1H1A.RRM2 | UHRF1 | 0.97 | CD.Fibro.ADAMDEC1.C7 | LAPTM4A | 1.51 | CD.Mono.CXCL3.FCN1 | FCN1 | 1.64 |
| CD.B/GC.HIST1H1A.RRM2 | AICDA | 0.96 | CD.Fibro.ADAMDEC1.C7 | CXCL1 | 1.51 | CD.Mono.CXCL3.FCN1 | IGSF6 | 1.51 |
| CD.B/GC.HIST1H1A.RRM2 | ESCO2 | 0.83 | CD.Fibro.ADAMDEC1.C7 | CTSK | 1.50 | CD.Mono.CXCL3.FCN1 | MNDA | 1.51 |
| CD.B/GC.HIST1H1A.RRM2 | HIST1H1B | 0.79 | CD.Fibro.ADAMDEC1.C7 | FHL1 | 1.50 | CD.Mono.CXCL3.FCN1 | CPVL | 1.50 |
| CD.B/GC.HIST1H1A.RRM2 | RAD51 | 0.70 | CD.Fibro.ADAMDEC1.C7 | RAB13 | 1.48 | CD.Mono.CXCL3.FCN1 | CLEC7A | 1.26 |
| CD.B/GC.HIST1H1A.RRM2 | RP11- | 0.67 | CD.Fibro.ADAMDEC1.C7 | CD63 | 1.47 | CD.Mono.CXCL3.FCN1 | AIF1 | 2.22 |
| | 132N15.3 | | | | | | | |
| CD.B/GC.HIST1H1A.RRM2 | E2F8 | 0.64 | CD.Fibro.ADAMDEC1.C7 | LGALS3BP | 1.46 | CD.Mono.CXCL3.FCN1 | MS4A6A | 1.67 |
| CD.B/GC.HIST1H1A.RRM2 | HIST1H1A | 0.59 | CD.Fibro.ADAMDEC1.C7 | VCAN | 1.45 | CD.Mono.CXCL3.FCN1 | NLRP3 | 0.92 |
| CD.B/GC.HIST1H1A.RRM2 | AP000251.3 | 0.32 | CD.Fibro.ADAMDEC1.C7 | NGFRAP1 | 1.44 | CD.Mono.CXCL3.FCN1 | FCGR1A | 0.92 |
| CD.B/GC.HIST1H1A.RRM2 | NUSAP1 | 1.53 | CD.Fibro.ADAMDEC1.C7 | ITGB1 | 1.44 | CD.Mono.CXCL3.FCN1 | CSTA | 1.00 |
| CD.B/GC.HIST1H1A.RRM2 | NMU | 0.49 | CD.Fibro.ADAMDEC1.C7 | FBLN5 | 1.41 | CD.Mono.FCN1.AREG | FCN1 | 2.59 |
| CD.B/GC.HIST1H1A.RRM2 | MND1 | 0.77 | CD.Fibro.ADAMDEC1.C7 | SGCE | 1.41 | CD.Mono.FCN1.HSPA1A | CD300E | 0.83 |
| CD.B/GC.HIST1H1A.RRM2 | KIAA0101 | 2.30 | CD.Fibro.ADAMDEC1.C7 | CLEC11A | 1.40 | CD.Mono.FCN1.LYST | LINC01272 | 2.57 |
| CD.B/GC.HIST1H1A.RRM2 | E2F1 | 0.64 | CD.Fibro.ADAMDEC1.C7 | PROS1 | 1.40 | CD.Mono.FCN1.S100A4 | AIF1 | 2.50 |
| CD.B/GC.HIST1H1A.RRM2 | C21orf58 | 0.69 | CD.Fibro.ADAMDEC1.C7 | PLAC9 | 1.39 | CD.Mono.FCN1.S100A4 | LST1 | 2.15 |
| CD.B/GC.HIST1H1A.RRM2 | AURKB | 0.94 | CD.Fibro.ADAMDEC1.C7 | COL6A1 | 1.35 | CD.Mono.FCN1.S100A4 | TYROBP | 2.13 |
| CD.B/GC.HIST1H1A.RRM2 | WDR76 | 1.03 | CD.Fibro.ADAMDEC1.C7 | CRISPLD2 | 1.35 | CD.Mono.FCN1.S100A4 | LYZ | 2.12 |
| CD.B/GC.HIST1H1A.RRM2 | CDCA7 | 1.36 | CD.Fibro.ADAMDEC1.C7 | IL6ST | 1.34 | CD.Mono.FCN1.S100A4 | FCN1 | 1.98 |
| CD.B/GC.HIST1H1A.RRM2 | PKMYT1 | 0.66 | CD.Fibro.ADAMDEC1.C7 | DDR2 | 1.32 | CD.Mono.FCN1.S100A4 | S100A9 | 1.95 |
| CD.B/GC.KIAA0101.GINS2 | HMGB2 | 2.39 | CD.Fibro.ADAMDEC1.C7 | NKX2-3 | 1.32 | CD.Mono.FCN1.S100A4 | MNDA | 1.90 |
| CD.B/GC.KIAA0101.GINS2 | STMN1 | 2.32 | CD.Fibro.ADAMDEC1.C7 | OGN | 1.32 | CD.Mono.FCN1.S100A4 | FTL | 1.89 |
| CD.B/GC.KIAA0101.GINS2 | TCL1A | 1.95 | CD.Fibro.ADAMDEC1.C7 | PCOLCE | 1.31 | CD.Mono.FCN1.S100A4 | FCER1G | 1.86 |
| CD.B/GC.KIAA0101.GINS2 | RGS13 | 1.90 | CD.Fibro.ADAMDEC1.C7 | FSTL1 | 1.30 | CD.Mono.FCN1.S100A4 | S100A8 | 1.79 |
| CD.B/GC.KIAA0101.GINS2 | PTTG1 | 1.87 | CD.Fibro.ADAMDEC1.C7 | COL6A3 | 1.29 | CD.Mono.FCN1.S100A4 | FGL2 | 1.76 |
| CD.B/GC.KIAA0101.GINS2 | KIAA0101 | 1.77 | CD.Fibro.ADAMDEC1.C7 | BMP5 | 1.29 | CD.Mono.FCN1.S100A4 | IL1B | 1.74 |
| CD.B/GC.KIAA0101.GINS2 | CD79B | 1.72 | CD.Fibro.ADAMDEC1.C7 | SFTA1P | 1.28 | CD.Mono.FCN1.S100A4 | PLAUR | 1.70 |
| CD.B/GC.KIAA0101.GINS2 | HMGA1 | 1.62 | CD.Fibro.ADAMDEC1.C7 | ZEB2 | 1.28 | CD.Mono.FCN1.S100A4 | CYBB | 1.60 |
| CD.B/GC.KIAA0101.GINS2 | C12orf75 | 1.35 | CD.Fibro.ADAMDEC1.C7 | CLEC14A | 1.27 | CD.Mono.FCN1.S100A4 | IGSF6 | 1.59 |
| CD.B/GC.KIAA0101.GINS2 | LRMP | 1.29 | CD.Fibro.ADAMDEC1.C7 | FN1 | 1.27 | CD.Mono.FCN1.S100A4 | MS4A6A | 1.49 |
| CD.B/GC.KIAA0101.GINS2 | TK1 | 1.21 | CD.Fibro.ADAMDEC1.C7 | FXYD1 | 1.26 | CD.Mono.FCN1.S100A4 | CST3 | 1.38 |
| CD.B/GC.KIAA0101.GINS2 | GINS2 | 1.20 | CD.Fibro.ADAMDEC1.C7 | SOD3 | 1.26 | CD.Mono.FCN1.S100A4 | EREG | 1.38 |
| CD.B/GC.KIAA0101.GINS2 | BIK | 1.16 | CD.Fibro.ADAMDEC1.C7 | FNDC1 | 1.26 | CD.Mono.FCN1.S100A4 | RP11- | 1.91 |
| | | | | | | | 1143G9.4 | |
| CD.B/GC.KIAA0101.GINS2 | AICDA | 1.15 | CD.Fibro.ADAMDEC1.C7 | IGFBP6 | 1.25 | CD.Mono.FCN1.S100A4 | OLR1 | 1.21 |
| CD.B/GC.KIAA0101.GINS2 | BCAS4 | 1.15 | CD.Fibro.ADAMDEC1.C7 | EPHX1 | 1.25 | CD.Mono.FCN1.S100A4 | SOD2 | 1.61 |
| CD.B/GC.KIAA0101.GINS2 | BCL7A | 1.15 | CD.Fibro.ADAMDEC1.C7 | PLAT | 1.24 | CD.Mono.FCN1.S100A4 | FTH1 | 1.32 |
| CD.B/GC.KIAA0101.GINS2 | CDCA7 | 1.15 | CD.Fibro.ADAMDEC1.C7 | MXRA8 | 1.24 | CD.Mono.FCN1.S100A4 | TMSB10 | 1.12 |
| CD.B/GC.KIAA0101.GINS2 | RP11- | 1.14 | CD.Fibro.ADAMDEC1.C7 | WBP5 | 1.24 | CD.Mono.S100A8.S100A9 | S100A8 | 4.68 |
| | 231C14.7 | | | | | | | |
| CD.B/GC.KIAA0101.GINS2 | MCM7 | 1.12 | CD.Fibro.ADAMDEC1.C7 | DKK3 | 1.23 | CD.Mono.S100A8.S100A9 | S100A9 | 4.56 |
| CD.B/GC.KIAA0101.GINS2 | DHFR | 1.08 | CD.Fibro.ADAMDEC1.C7 | DPT | 1.23 | CD.Mono.S100A8.S100A9 | RP11- | 3.06 |
| | | | | | | | 1143G9.4 | |
| CD.B/GC.KIAA0101.GINS2 | NEIL1 | 1.04 | CD.Fibro.ADAMDEC1.C7 | SPRY1 | 1.23 | CD.Mono.S100A8.S100A9 | FCN1 | 2.66 |
| CD.B/GC.KIAA0101.GINS2 | SUGCT | 1.04 | CD.Fibro.ADAMDEC1.C7 | LOXL1 | 1.22 | CD.Mono.S100A8.S100A9 | SOD2 | 2.56 |
| CD.B/GC.KIAA0101.GINS2 | BIRC5 | 1.04 | CD.Fibro.ADAMDEC1.C7 | CP | 1.21 | CD.Mono.S100A8.S100A9 | S100A12 | 2.44 |
| CD.B/GC.KIAA0101.GINS2 | RASSF6 | 1.04 | CD.Fibro.ADAMDEC1.C7 | LINC01082 | 1.21 | CD.Mono.S100A8.S100A9 | IL1B | 2.39 |
| CD.B/GC.KIAA0101.GINS2 | CENPF | 1.03 | CD.Fibro.ADAMDEC1.C7 | SELM | 1.21 | CD.Mono.S100A8.S100A9 | LYZ | 2.36 |
| CD.B/GC.KIAA0101.GINS2 | DAAM1 | 1.02 | CD.Fibro.ADAMDEC1.C7 | NFIA | 1.21 | CD.Mono.S100A8.S100A9 | VCAN | 2.35 |
| CD.B/GC.KIAA0101.GINS2 | RMI2 | 0.99 | CD.Fibro.ADAMDEC1.C7 | PLEKHH2 | 1.20 | CD.Mono.S100A8.S100A9 | TIMP1 | 2.31 |
| CD.B/GC.KIAA0101.GINS2 | CDC20 | 0.98 | CD.Fibro.ADAMDEC1.C7 | PTMS | 1.19 | CD.Mono.S100A8.S100A9 | MNDA | 2.14 |
| CD.B/GC.KIAA0101.GINS2 | CDKN3 | 0.97 | CD.Fibro.ADAMDEC1.C7 | TGFBI | 1.18 | CD.Mono.S100A8.S100A9 | TYROBP | 2.10 |
| CD.B/GC.KIAA0101.GINS2 | WDR66 | 0.96 | CD.Fibro.ADAMDEC1.C7 | FILIP1L | 1.17 | CD.Mono.S100A8.S100A9 | NAMPT | 2.08 |
| CD.B/GC.KIAA0101.GINS2 | CENPM | 0.96 | CD.Fibro.ADAMDEC1.C7 | CD302 | 1.17 | CD.Mono.S100A8.S100A9 | AIF1 | 2.07 |
| CD.B/GC.KIAA0101.GINS2 | MKI67 | 0.96 | CD.Fibro.ADAMDEC1.C7 | EFEMP1 | 1.17 | CD.Mono.S100A8.S100A9 | S100A4 | 2.05 |
| CD.B/GC.KIAA0101.GINS2 | NUSAP1 | 0.94 | CD.Fibro.ADAMDEC1.C7 | TNFRSF1A | 1.17 | CD.Mono.S100A8.S100A9 | GOS2 | 2.00 |
| CD.B/GC.KIAA0101.GINS2 | HELLS | 0.92 | CD.Fibro.ADAMDEC1.C7 | NDN | 1.16 | CD.Mono.S100A8.S100A9 | CD14 | 1.98 |
| CD.B/GC.KIAA0101.GINS2 | SPIB | 0.90 | CD.Fibro.ADAMDEC1.C7 | MATN2 | 1.14 | CD.Mono.S100A8.S100A9 | FCER1G | 1.89 |
| CD.B/GC.KIAA0101.GINS2 | CDT1 | 0.89 | CD.Fibro.ADAMDEC1.C7 | CNN3 | 1.14 | CD.Mono.S100A8.S100A9 | LST1 | 1.85 |
| CD.B/GC.KIAA0101.GINS2 | GCSAM | 0.87 | CD.Fibro.ADAMDEC1.C7 | EFEMP2 | 1.13 | CD.Mono.S100A8.S100A9 | PLEK | 1.76 |
| CD.B/GC.KIAA0101.GINS2 | TYMS | 0.87 | CD.Fibro.ADAMDEC1.C7 | SDC2 | 1.12 | CD.Mono.S100A8.S100A9 | S100A11 | 1.75 |
| CD.B/GC.KIAA0101.GINS2 | AC023590.1 | 0.82 | CD.Fibro.ADAMDEC1.C7 | SERPINH1 | 1.12 | CD.Mono.S100A8.S100A9 | CTSS | 1.69 |
| CD.B/GC.KIAA0101.GINS2 | S1PR2 | 0.81 | CD.Fibro.ADAMDEC1.C7 | MYL9 | 1.12 | CD.Mono.S100A8.S100A9 | SERPINA1 | 1.69 |
| CD.B/GC.KIAA0101.GINS2 | SERPINA9 | 0.81 | CD.Fibro.ADAMDEC1.C7 | TDO2 | 1.11 | CD.Mono.S100A8.S100A9 | BCL2A1 | 1.65 |
| CD.B/GC.KIAA0101.GINS2 | MYBL2 | 0.80 | CD.Fibro.ADAMDEC1.C7 | CEBPD | 1.11 | CD.Mono.S100A8.S100A9 | FGL2 | 1.62 |
| CD.B/GC.KIAA0101.GINS2 | CENPU | 0.79 | CD.Fibro.ADAMDEC1.C7 | LEPROT | 1.11 | CD.Mono.S100A8.S100A9 | CYBB | 1.59 |
| CD.B/GC.KIAA0101.GINS2 | CLSPN | 0.78 | CD.Fibro.ADAMDEC1.C7 | SMPDL3A | 1.10 | CD.Mono.S100A8.S100A9 | PLAUR | 1.59 |
| CD.B/GC.KIAA0101.GINS2 | MAD2L1 | 0.75 | CD.Fibro.ADAMDEC1.C7 | CCDC80 | 1.10 | CD.Mono.S100A8.S100A9 | DUSP6 | 1.57 |
| CD.B/GC.KIAA0101.GINS2 | WDR76 | 0.73 | CD.Fibro.ADAMDEC1.C7 | ECM1 | 1.10 | CD.Mono.S100A8.S100A9 | MAFB | 1.42 |
| CD.B/GC.KIAA0101.GINS2 | MND1 | 0.73 | CD.Fibro.ADAMDEC1.C7 | SLC9A3R2 | 1.09 | CD.Mono.S100A8.S100A9 | LINC01272 | 1.39 |
| CD.B/GC.KIAA0101.GINS2 | RP11- | 0.73 | CD.Fibro.ADAMDEC1.C7 | IGFBP4 | 1.09 | CD.Mono.S100A8.S100A9 | EREG | 1.37 |
| | 132N15.3 | | | | | | | |
| CD.B/GC.KIAA0101.GINS2 | MSH6 | 0.73 | CD.Fibro.ADAMDEC1.C7 | CRYBG3 | 1.07 | CD.Mono.S100A8.S100A9 | IL1RN | 1.33 |
| CD.B/GC.KIAA0101.GINS2 | DCAF12 | 0.72 | CD.Fibro.ADAMDEC1.C7 | FHL2 | 1.07 | CD.Mono.S100A8.S100A9 | MS4A6A | 1.32 |
| CD.B/GC.KIAA0101.GINS2 | ASF1B | 0.69 | CD.Fibro.ADAMDEC1.C7 | ITIH5 | 1.07 | CD.Mono.S100A8.S100A9 | FPR1 | 1.32 |
| CD.B/GC.KIAA0101.GINS2 | ZWINT | 0.69 | CD.Fibro.ADAMDEC1.C7 | IL34 | 1.06 | CD.Mono.S100A8.S100A9 | CSTA | 1.30 |
| CD.B/GC.KIAA0101.GINS2 | MYBL1 | 0.65 | CD.Fibro.ADAMDEC1.C7 | SERPINE2 | 1.06 | CD.Mono.S100A8.S100A9 | CD300E | 1.20 |
| CD.B/GC.KIAA0101.GINS2 | BRCA1 | 0.64 | CD.Fibro.ADAMDEC1.C7 | PRNP | 1.05 | CD.Mono.S100A8.S100A9 | CFP | 1.18 |
| CD.B/GC.KIAA0101.GINS2 | TUBB2B | 0.53 | CD.Fibro.ADAMDEC1.C7 | COL18A1 | 1.05 | CD.Mono.S100A8.S100A9 | AQP9 | 1.18 |
| CD.B/GC.KIAA0101.GINS2 | HRK | 0.52 | CD.Fibro.ADAMDEC1.C7 | TSPAN4 | 1.05 | CD.Mono.S100A8.S100A9 | APOBEC3A | 1.11 |
| CD.B/GC.KIAA0101.GINS2 | E2F1 | 0.51 | CD.Fibro.ADAMDEC1.C7 | BCHE | 1.05 | CD.Mono.S100A8.S100A9 | FCGR1A | 1.10 |
| CD.B/GC.KIAA0101.GINS2 | CCNB2 | 0.94 | CD.Fibro.ADAMDEC1.C7 | UBE2E3 | 1.05 | CD.Mono.S100A8.S100A9 | LILRA5 | 1.04 |
| CD.B/GC.KIAA0101.GINS2 | TOP2A | 0.88 | CD.Fibro.ADAMDEC1.C7 | PDGFRA | 1.03 | CD.Mono.S100A8.S100A9 | TREM1 | 1.03 |
| CD.B/GC.KIAA0101.GINS2 | RFTN1 | 0.84 | CD.Fibro.ADAMDEC1.C7 | COL14A1 | 1.03 | CD.Mono.S100A8.S100A9 | FOLR3 | 0.84 |
| CD.B/GC.KIAA0101.GINS2 | UHRF1 | 0.57 | CD.Fibro.ADAMDEC1.C7 | BMP4 | 1.03 | CD.Mono.S100A8.S100A9 | NRG1 | 0.82 |
| CD.B/GC.KIAA0101.GINS2 | MCM4 | 0.59 | CD.Fibro.ADAMDEC1.C7 | C1orf21 | 1.03 | CD.Mono.S100A8.S100A9 | MCEMP1 | 0.74 |
| CD.B/GC.KIAA0101.GINS2 | FCRLA | 0.79 | CD.Fibro.ADAMDEC1.C7 | GNG11 | 1.02 | CD.Mono.S100A8.S100A9 | TYMP | 1.87 |
| CD.B/GC.KIAA0101.GINS2 | RFC3 | 0.53 | CD.Fibro.ADAMDEC1.C7 | GSTM3 | 1.01 | CD.Mono.S100A8.S100A9 | ACSL1 | 1.13 |
| CD.B/GC.KIAA0101.GINS2 | FEN1 | 0.73 | CD.Fibro.ADAMDEC1.C7 | PRKCDBP | 1.00 | CD.Mono.S100A8.S100A9 | SPI1 | 1.38 |
| CD.B/GC.KIAA0101.GINS2 | BASP1 | 1.09 | CD.Fibro.ADAMDEC1.C7 | FXYD6 | 0.99 | CD.Mono.S100A8.S100A9 | FTH1 | 1.54 |
| CD.B/GC.KIAA0101.GINS2 | SUSD3 | 0.95 | CD.Fibro.ADAMDEC1.C7 | TIMP1 | 0.98 | CD.Mono.S100A8.S100A9 | FGR | 0.89 |
| CD.B/GC.KIAA0101.GINS2 | BLK | 0.67 | CD.Fibro.ADAMDEC1.C7 | COX7A1 | 0.97 | CD.Mono.S100A8.S100A9 | CCL3 | 2.14 |
| CD.B/GC.KIAA0101.GINS2 | TFDP1 | 0.85 | CD.Fibro.ADAMDEC1.C7 | GULP1 | 0.97 | CD.Mono.S100A8.S100A9 | CXCL10 | 2.04 |
| CD.B/GC.KIAA0101.GINS2 | GCHFR | 1.02 | CD.Fibro.ADAMDEC1.C7 | TPM2 | 0.96 | CD.Mono.S100A8.S100A9 | FTL | 1.35 |
| CD.B/GC.KIAA0101.GINS2 | RP11- | 0.81 | CD.Fibro.ADAMDEC1.C7 | EHD2 | 0.96 | CD.Mono/cDC2.CLEC10A.AREG | CLEC10A | 2.42 |
| | 620J15.3 | | | | | | | |
| CD.B/GC.KIAA0101.GINS2 | CENPH | 0.64 | CD.Fibro.ADAMDEC1.C7 | TMEM98 | 0.95 | CD.Mono/cDC2.CLEC10A.AREG | VSIG4 | 1.80 |
| CD.B/GC.KIAA0101.GINS2 | ORC6 | 0.58 | CD.Fibro.ADAMDEC1.C7 | CPNE8 | 0.95 | CD.Mono/cDC2.CLEC10A.SDS | VSIG4 | 1.52 |
| CD.B/GC.MKI67.RRM2 | SPC25 | 0.96 | CD.Fibro.ADAMDEC1.C7 | TSC22D1 | 0.95 | CD.Mono/cDC2.CLEC10A.SDS | SDS | 1.47 |
| CD.B/GC.RRM2.TK1 | TK1 | 2.02 | CD.Fibro.ADAMDEC1.C7 | RBP5 | 0.94 | CD.Mono/cDC2.CLEC10A.SDS | FCER1A | 1.38 |
| CD.B/GC.RRM2.TK1 | RRM2 | 1.88 | CD.Fibro.ADAMDEC1.C7 | THY1 | 0.94 | CD.Mono/cDC2.CLEC10A.SDS | HLA-DQB2 | 1.14 |
| CD.B/GC.RRM2.TK1 | TYMS | 1.75 | CD.Fibro.ADAMDEC1.C7 | PPIC | 0.94 | CD.Mono/cDC2.CLEC10A.SDS | CD1E | 1.13 |
| CD.B/GC.RRM2.TK1 | MKI67 | 1.72 | CD.Fibro.ADAMDEC1.C7 | NFIC | 0.93 | CD.Mono/cDC2.CLEC10A.SDS | CSF2RA | 0.99 |
| CD.B/GC.RRM2.TK1 | CDK1 | 1.70 | CD.Fibro.ADAMDEC1.C7 | MFGE8 | 0.93 | CD.Mono/cDC2.CLEC10A.SDS | RP11- | 0.41 |
| | | | | | | | 404013.5 | |
| CD.B/GC.RRM2.TK1 | UBE2C | 1.58 | CD.Fibro.ADAMDEC1.C7 | IGFBP5 | 0.93 | CD.Mono/cDC2.CLEC10A.SDS | CLEC4F | 0.26 |
| CD.B/GC.RRM2.TK1 | RMI2 | 1.47 | CD.Fibro.ADAMDEC1.C7 | RBPMS | 0.93 | CD.Mono/cDC2.CLEC10A.SDS | PID1 | 0.66 |
| CD.B/GC.RRM2.TK1 | BIRC5 | 1.36 | CD.Fibro.ADAMDEC1.C7 | FOXF1 | 0.93 | CD.Mono/cDC2.CLEC10A.SDS | RAB7B | 0.52 |
| CD.B/GC.RRM2.TK1 | AURKB | 1.36 | CD.Fibro.ADAMDEC1.C7 | MMP14 | 0.92 | CD.Mono/cDC2.CLEC10A.SDS | ITGAX | 0.89 |
| CD.B/GC.RRM2.TK1 | CDT1 | 1.26 | CD.Fibro.ADAMDEC1.C7 | CYBRD1 | 0.92 | CD.Mono/cDC2.CLEC10A.SDS | PLD4 | 1.20 |
| CD.B/GC.RRM2.TK1 | ASF1B | 1.24 | CD.Fibro.ADAMDEC1.C7 | NDRG2 | 0.92 | CD.Mono/cDC2.FCN1.CD1C | FCN1 | 2.04 |
| CD.B/GC.RRM2.TK1 | SPC25 | 1.18 | CD.Fibro.ADAMDEC1.C7 | PCDH7 | 0.92 | CD.Mono/cDC2.FCN1.CD1C | FCER1A | 1.78 |
| CD.B/GC.RRM2.TK1 | MYBL2 | 1.11 | CD.Fibro.ADAMDEC1.C7 | ALDH1A3 | 0.92 | CD.Mono/Mac.CXCL10.CXCL11 | CXCL10 | 3.62 |
| CD.B/GC.RRM2.TK1 | FAM111B | 1.01 | CD.Fibro.ADAMDEC1.C7 | ABCA6 | 0.90 | CD.Mono/Mac.CXCL10.CXCL11 | CXCL9 | 2.94 |
| CD.B/GC.RRM2.TK1 | MXD3 | 1.01 | CD.Fibro.ADAMDEC1.C7 | TNC | 0.90 | CD.Mono/Mac.CXCL10.FCN1 | CXCL10 | 3.63 |
| CD.B/GC.RRM2.TK1 | CDCA5 | 1.01 | CD.Fibro.ADAMDEC1.C7 | PCDH18 | 0.90 | CD.Mono/Mac.CXCL10.FCN1 | CXCL9 | 2.48 |
| CD.B/GC.RRM2.TK1 | PBK | 1.00 | CD.Fibro.ADAMDEC1.C7 | STMN2 | 0.90 | CD.Mono/Mac.CXCL10.FCN1 | FCN1 | 2.06 |
| CD.B/GC.RRM2.TK1 | AICDA | 0.94 | CD.Fibro.ADAMDEC1.C7 | FRZB | 0.90 | CD.Mono/Mac.CXCL10.FCN1 | S100A9 | 1.94 |
| CD.B/GC.RRM2.TK1 | KIFC1 | 0.92 | CD.Fibro.ADAMDEC1.C7 | COL5A2 | 0.89 | CD.Mono/Mac.CXCL10.FCN1 | S100A8 | 1.54 |
| CD.B/GC.RRM2.TK1 | CCNA2 | 0.82 | CD.Fibro.ADAMDEC1.C7 | SFRP1 | 0.89 | CD.Mono/Mac.CXCL10.GBP1 | CXCL10 | 3.61 |
| CD.B/GC.RRM2.TK1 | RFC3 | 0.79 | CD.Fibro.ADAMDEC1.C7 | WFDC1 | 0.88 | CD.Mono/Mac.CXCL10.GBP1 | CXCL9 | 2.58 |
| CD.B/GC.RRM2.TK1 | RP11- | 0.64 | CD.Fibro.ADAMDEC1.C7 | PTCH1 | 0.88 | CD.Mstcl.AREG.ADCYAP1 | TPSAB1 | 4.12 |
| | 132N15.3 | | | | | | | |
| CD.B/GC.RRM2.TK1 | RAD51 | 0.62 | CD.Fibro.ADAMDEC1.C7 | C2 | 0.88 | CD.Mstcl.AREG.ADCYAP1 | TPSB2 | 3.82 |
| CD.B/GC.RRM2.TK1 | NCAPH | 0.57 | CD.Fibro.ADAMDEC1.C7 | NNMT | 0.86 | CD.Mstcl.AREG.ADCYAP1 | CPA3 | 3.35 |
| CD.B/GC.RRM2.TK1 | KIF2C | 0.68 | CD.Fibro.ADAMDEC1.C7 | PLXDC2 | 0.86 | CD.Mstcl.AREG.ADCYAP1 | MS4A2 | 2.57 |
| CD.B/GC.RRM2.TK1 | BCL7A | 1.03 | CD.Fibro.ADAMDEC1.C7 | EDNRB | 0.86 | CD.Mstcl.AREG.ADCYAP1 | HPGDS | 2.44 |
| CD.B/GC.RRM2.TK1 | NUSAP1 | 1.71 | CD.Fibro.ADAMDEC1.C7 | COL15A1 | 0.85 | CD.Mstcl.AREG.ADCYAP1 | IL13 | 2.23 |
| CD.B/GC.RRM2.TK1 | CLSPN | 1.14 | CD.Fibro.ADAMDEC1.C7 | PDK4 | 0.85 | CD.Mstcl.AREG.ADCYAP1 | CTSG | 2.15 |
| CD.B/GC.RRM2.TK1 | CDCA3 | 0.95 | CD.Fibro.ADAMDEC1.C7 | OAF | 0.85 | CD.Mstcl.AREG.ADCYAP1 | ADCYAP1 | 2.04 |
| CD.B/GC.RRM2.TK1 | DHFR | 1.43 | CD.Fibro.ADAMDEC1.C7 | NID1 | 0.84 | CD.Mstcl.AREG.ADCYAP1 | HDC | 2.04 |
| CD.B/GC.RRM2.TK1 | RAD54L | 0.41 | CD.Fibro.ADAMDEC1.C7 | ADIRF | 0.84 | CD.Mstcl.AREG.ADCYAP1 | VWA5A | 1.98 |
| CD.B/GC.RRM2.TK1 | FBXO5 | 1.06 | CD.Fibro.ADAMDEC1.C7 | CPQ | 0.84 | CD.Mstcl.AREG.ADCYAP1 | SLC18A2 | 1.98 |
| CD.B/GC.RRM2.TK1 | MND1 | 0.72 | CD.Fibro.ADAMDEC1.C7 | GPC6 | 0.83 | CD.Mstcl.AREG.ADCYAP1 | KIT | 1.94 |
| CD.B/GC.RRM2.TK1 | CDKN3 | 1.27 | CD.Fibro.ADAMDEC1.C7 | KCNS3 | 0.83 | CD.Mstcl.AREG.ADCYAP1 | IL1RL1 | 1.92 |
| CD.B/GC.RRM2.TK1 | UBE2T | 1.05 | CD.Fibro.ADAMDEC1.C7 | TGFB111 | 0.83 | CD.Mstcl.AREG.ADCYAP1 | GATA2 | 1.84 |
| CD.B/GC.RRM2.TK1 | NUF2 | 0.86 | CD.Fibro.ADAMDEC1.C7 | TRIL | 0.83 | CD.Mstcl.AREG.ADCYAP1 | AC004791.2 | 1.48 |
| CD.B/GC.RRM2.TK1 | ZWINT | 1.31 | CD.Fibro.ADAMDEC1.C7 | TNXB | 0.83 | CD.Mstcl.AREG.ADCYAP1 | RP11- | 1.44 |
| | | | | | | | 354E11.2 | |
| CD.B/GC.RRM2.TYMS | CLSPN | 1.10 | CD.Fibro.ADAMDEC1.C7 | AKAP12 | 0.83 | CD.Mstcl.AREG.ADCYAP1 | LTC4S | 1.55 |
| CD.B/GC.RRM2.TYMS | MYBL2 | 1.01 | CD.Fibro.ADAMDEC1.C7 | AXL | 0.82 | CD.Mstcl.CD3D.CD2 | CPA3 | 2.23 |
| CD.B/GC.RRM2.TYMS | ASF1B | 0.81 | CD.Fibro.ADAMDEC1.C7 | KITLG | 0.82 | CD.Mstcl.CDK1.KIAA0101 | SLC18A2 | 2.10 |
| CD.B/GC.RRM2.TYMS | SERPINA9 | 0.78 | CD.Fibro.ADAMDEC1.C7 | MMP23B | 0.82 | CD.Mstcl.CDK1.KIAA0101 | MS4A2 | 2.31 |
| CD.B/GC.RRM2.TYMS | MND1 | 0.75 | CD.Fibro.ADAMDEC1.C7 | GSTM5 | 0.82 | CD.Mstcl.CDK1.KIAA0101 | KIT | 1.95 |
| CD.B/GC.RRM2.TYMS | GTSE1 | 0.65 | CD.Fibro.ADAMDEC1.C7 | LAMA4 | 0.82 | CD.Mstcl.CTSG.CD69 | TPSAB1 | 4.44 |
| CD.B/GC.RRM2.TYMS | CDCA5 | 0.59 | CD.Fibro.ADAMDEC1.C7 | WNT2B | 0.82 | CD.Mstcl.CTSG.CD69 | TPSB2 | 4.40 |
| CD.B/GC.RRM2.TYMS | HIST1H1A | 0.53 | CD.Fibro.ADAMDEC1.C7 | PAM | 0.81 | CD.Mstcl.CTSG.CD69 | CTSG | 3.79 |
| CD.B/GC.RRM2.TYMS | UHRF1 | 0.51 | CD.Fibro.ADAMDEC1.C7 | AEBP1 | 0.80 | CD.Mstcl.CTSG.CD69 | HPGDS | 3.09 |
| CD.B/GC.RRM2.TYMS | CDC45 | 0.49 | CD.Fibro.ADAMDEC1.C7 | COL12A1 | 0.79 | CD.Mstcl.CTSG.CD69 | CPA3 | 2.72 |
| CD.B/GC.RRM2.TYMS | E2F1 | 0.48 | CD.Fibro.ADAMDEC1.C7 | ABI3BP | 0.79 | CD.Mstcl.CTSG.CD69 | ANXA1 | 2.62 |
| CD.B/GC.RRM2.TYMS | SKA3 | 0.40 | CD.Fibro.ADAMDEC1.C7 | MAB21L2 | 0.79 | CD.Mstcl.CTSG.CD69 | HPGD | 2.50 |
| CD.B/GC.RRM2.TYMS | NMU | 0.35 | CD.Fibro.ADAMDEC1.C7 | NEGR1 | 0.79 | CD.Mstcl.CTSG.CD69 | MS4A2 | 2.47 |
| CD.B/GC.RRM2.TYMS | RRM2 | 1.46 | CD.Fibro.ADAMDEC1.C7 | LRP1 | 0.78 | CD.Mstcl.CTSG.CD69 | AC020571.3 | 2.41 |
| CD.B/GC.RRM2.TYMS | TYMS | 1.43 | CD.Fibro.ADAMDEC1.C7 | ZNF503 | 0.77 | CD.Mstcl.CTSG.CD69 | CD9 | 2.17 |
| CD.B/GC.RRM2.ZWINT | SPC25 | 1.05 | CD.Fibro.ADAMDEC1.C7 | TPPP3 | 0.77 | CD.Mstcl.CTSG.CD69 | VWA5A | 2.15 |
| CD.B/GC.RRM2.ZWINT | RP11- | 0.92 | CD.Fibro.ADAMDEC1.C7 | CCDC102B | 0.76 | CD.Mstcl.CTSG.CD69 | CMA1 | 2.15 |
| | 164H13.1 | | | | | | | |
| CD.B/GC.RRM2.ZWINT | PBK | 0.92 | CD.Fibro.ADAMDEC1.C7 | GRK5 | 0.76 | CD.Mstcl.CTSG.CD69 | RGS13 | 2.14 |
| CD.B/GC.RRM2.ZWINT | SERPINA9 | 0.89 | CD.Fibro.ADAMDEC1.C7 | FKBP10 | 0.76 | CD.Mstcl.CTSG.CD69 | HDC | 2.09 |
| CD.B/GC.RRM2.ZWINT | TUBB2B | 0.88 | CD.Fibro.ADAMDEC1.C7 | NOVA1 | 0.75 | CD.Mstcl.CTSG.CD69 | KIT | 2.05 |
| CD.B/GC.RRM2.ZWINT | UHRF1 | 0.87 | CD.Fibro.ADAMDEC1.C7 | GLT8D2 | 0.75 | CD.Mstcl.CTSG.CD69 | NFKBIA | 2.00 |
| CD.B/GC.RRM2.ZWINT | NCAPG | 0.87 | CD.Fibro.ADAMDEC1.C7 | PTK7 | 0.74 | CD.Mstcl.CTSG.CD69 | SLC18A2 | 1.93 |
| CD.B/GC.RRM2.ZWINT | RP11- | 0.65 | CD.Fibro.ADAMDEC1.C7 | FAM198B | 0.74 | CD.Mstcl.CTSG.CD69 | KRT1 | 1.90 |
| | 132N15.3 | | | | | | | |
| CD.B/GC.RRM2.ZWINT | WDR66 | 0.63 | CD.Fibro.ADAMDEC1.C7 | HOXC8 | 0.74 | CD.Mstcl.CTSG.CD69 | IL1RL1 | 1.89 |
| CD.B/GC.RRM2.ZWINT | NEIL3 | 0.57 | CD.Fibro.ADAMDEC1.C7 | PLAU | 0.74 | CD.Mstcl.CTSG.CD69 | CLU | 1.83 |
| CD.B/GC.RRM2.ZWINT | FAM81A | 0.38 | CD.Fibro.ADAMDEC1.C7 | DIO2 | 0.74 | CD.Mstcl.CTSG.CD69 | MAOB | 1.62 |
| CD.B/GC.RRM2.ZWINT | AURKB | 1.10 | CD.Fibro.ADAMDEC1.C7 | HOXC9 | 0.73 | CD.Mstcl.CTSG.CD69 | FCER1A | 1.54 |
| CD.B/GC.RRM2.ZWINT | RAD54L | 0.32 | CD.Fibro.ADAMDEC1.C7 | TMEM119 | 0.72 | CD.Mstcl.CTSG.CD69 | LTC4S | 1.51 |
| CD.B/GC.RRM2.ZWINT | RRM2 | 1.80 | CD.Fibro.ADAMDEC1.C7 | MAMDC2 | 0.72 | CD.Mstcl.CTSG.CD69 | FTH1 | 1.49 |
| CD.B/GC.RRM2.ZWINT | WDR76 | 0.95 | CD.Fibro.ADAMDEC1.C7 | CLMP | 0.72 | CD.Mstcl.CTSG.CD69 | IL1RAPL1 | 1.41 |
| CD.B/GC.RRM2.ZWINT | E2F1 | 0.64 | CD.Fibro.ADAMDEC1.C7 | SRPX | 0.71 | CD.Mstcl.CTSG.CD69 | RP11- | 1.38 |
| | | | | | | | 354E11.2 | |
| CD.B/GC.TYMS.MKI67 | TYMS | 1.67 | CD.Fibro.ADAMDEC1.C7 | SCARA5 | 0.71 | CD.Mstcl.CTSG.CD69 | LMNA | 2.24 |
| CD.B/GC.TYMS.MKI67 | SERPINA9 | 0.91 | CD.Fibro.ADAMDEC1.C7 | ISLR | 0.70 | CD.Mstcl.CTSG.CD69 | CD69 | 2.20 |
| CD.B/LZ.CCL22.NPW | NPW | 0.70 | CD.Fibro.ADAMDEC1.C7 | SLIT2 | 0.70 | CD.Mstcl.DEFA5.RPLP2 | TPSB2 | 3.49 |
| CD.Bclls.CD69.GPR183 | VPREB3 | 1.40 | CD.Fibro.ADAMDEC1.C7 | EMID1 | 0.70 | CD.Mstcl.DEFA5.RPLP2 | TPSAB1 | 3.42 |
| CD.Bclls.CD69.GPR183 | MS4A1 | 1.39 | CD.Fibro.ADAMDEC1.C7 | MYLK | 0.69 | CD.Mstcl.DEFA5.RPLP2 | CPA3 | 2.83 |
| CD.Bclls.CD69.GPR183 | CD83 | 1.35 | CD.Fibro.ADAMDEC1.C7 | KANK2 | 0.69 | CD.Mstcl.DEFA5.RPLP2 | HPGDS | 2.31 |
| CD.Bclls.CD69.GPR183 | BANK1 | 1.29 | CD.Fibro.ADAMDEC1.C7 | SHISA3 | 0.69 | CD.Mstcl.DEFA5.RPLP2 | MS4A2 | 2.13 |
| CD.Bclls.CD69.GPR183 | CD79A | 1.07 | CD.Fibro.ADAMDEC1.C7 | HAS2 | 0.68 | CD.Mstcl.DEFA5.RPLP2 | GATA2 | 2.01 |
| CD.Bclls.CD69.GPR183 | HLA-DQB1 | 0.92 | CD.Fibro.ADAMDEC1.C7 | FGF7 | 0.68 | CD.Mstcl.DEFA5.RPLP2 | IL13 | 1.90 |
| CD.Bclls.CD69.GPR183 | CD74 | 0.90 | CD.Fibro.ADAMDEC1.C7 | ADGRL3 | 0.67 | CD.Mstcl.DEFA5.RPLP2 | HDC | 1.82 |
| CD.Bclls.CD69.GPR183 | HLA-DRA | 0.67 | CD.Fibro.ADAMDEC1.C7 | NR2F1 | 0.66 | CD.Mstcl.DEFA5.RPLP2 | CLU | 1.81 |
| CD.Bclls.CD69.GPR183 | CD69 | 1.07 | CD.Fibro.ADAMDEC1.C7 | FGFR4 | 0.66 | CD.Mstcl.DEFA5.RPLP2 | VWA5A | 1.75 |
| CD.Bclls.CD69.GPR183 | RPS27 | 0.56 | CD.Fibro.ADAMDEC1.C7 | MXRA5 | 0.66 | CD.Mstcl.DEFA5.RPLP2 | SLC18A2 | 1.25 |
| CD.cDC1.CLEC9A.IDO1 | IDO1 | 2.42 | CD.Fibro.ADAMDEC1.C7 | TMEM47 | 0.65 | CD.Mstcl.DEFA5.RPLP2 | RP11- | 1.10 |
| | | | | | | | 354E11.2 | |
| CD.cDC1.CLEC9A.IDO1 | CLEC9A | 2.26 | CD.Fibro.ADAMDEC1.C7 | NDUFA4L2 | 0.64 | CD.Mstcl.DEFA5.RPLP2 | HPGD | 1.83 |
| CD.cDC1.CLEC9A.IDO1 | C1orf54 | 2.14 | CD.Fibro.ADAMDEC1.C7 | FENDRR | 0.64 | CD.Mstcl.FOS.JUN | TPSB2 | 3.98 |
| CD.cDC1.CLEC9A.XCR1 | CPVL | 2.70 | CD.Fibro.ADAMDEC1.C7 | HTRA1 | 0.64 | CD.Mstcl.FOS.JUN | TPSAB1 | 3.93 |
| CD.cDC1.CLEC9A.XCR1 | CST3 | 2.64 | CD.Fibro.ADAMDEC1.C7 | TPST1 | 0.64 | CD.Mstcl.FOS.JUN | CTSG | 3.17 |
| CD.cDC1.CLEC9A.XCR1 | IDO1 | 2.36 | CD.Fibro.ADAMDEC1.C7 | FIBIN | 0.64 | CD.Mstcl.FOS.JUN | CPA3 | 2.78 |
| CD.cDC1.CLEC9A.XCR1 | HLA-DPB1 | 2.20 | CD.Fibro.ADAMDEC1.C7 | APOD | 0.63 | CD.Mstcl.FOS.JUN | HPGDS | 2.77 |
| CD.cDC1.CLEC9A.XCR1 | DNASE1L3 | 2.19 | CD.Fibro.ADAMDEC1.C7 | COL6A5 | 0.63 | CD.Mstcl.FOS.JUN | MS4A2 | 2.62 |
| CD.cDC1.CLEC9A.XCR1 | CLEC9A | 2.19 | CD.Fibro.ADAMDEC1.C7 | CDH11 | 0.63 | CD.Mstcl.FOS.JUN | VWA5A | 2.17 |
| CD.cDC1.CLEC9A.XCR1 | C1orf54 | 2.16 | CD.Fibro.ADAMDEC1.C7 | CNRIP1 | 0.63 | CD.Mstcl.FOS.JUN | SLC18A2 | 2.07 |
| CD.cDC1.CLEC9A.XCR1 | HLA-DQB1 | 1.97 | CD.Fibro.ADAMDEC1.C7 | TUSC3 | 0.62 | CD.Mstcl.FOS.JUN | GATA2 | 2.04 |
| CD.cDC1.CLEC9A.XCR1 | HLA-DQA1 | 1.95 | CD.Fibro.ADAMDEC1.C7 | COL5A1 | 0.61 | CD.Mstcl.FOS.JUN | KIT | 2.03 |
| CD.cDC1.CLEC9A.XCR1 | SNX3 | 1.94 | CD.Fibro.ADAMDEC1.C7 | TNS2 | 0.61 | CD.Mstcl.FOS.JUN | HDC | 1.84 |
| CD.cDC1.CLEC9A.XCR1 | LGALS2 | 1.87 | CD.Fibro.ADAMDEC1.C7 | COLEC11 | 0.60 | CD.Mstcl.HSPA1A.HSP90AA1 | TPSAB1 | 3.64 |
| CD.cDC1.CLEC9A.XCR1 | SGK1 | 1.69 | CD.Fibro.ADAMDEC1.C7 | SCT | 0.60 | CD.Mstcl.HSPA1A.HSP90AA1 | TPSB2 | 3.24 |
| CD.cDC1.CLEC9A.XCR1 | CPNE3 | 1.66 | CD.Fibro.ADAMDEC1.C7 | PTGES | 0.59 | CD.Mstcl.HSPA1A.HSP90AA1 | CPA3 | 2.74 |
| CD.cDC1.CLEC9A.XCR1 | PPT1 | 1.59 | CD.Fibro.ADAMDEC1.C7 | OLFML1 | 0.59 | CD.Mstcl.HSPA1A.HSP90AA1 | CTSG | 2.68 |
| CD.cDC1.CLEC9A.XCR1 | LYZ | 1.57 | CD.Fibro.ADAMDEC1.C7 | PDGFRB | 0.58 | CD.Mstcl.HSPA1A.HSP90AA1 | HPGDS | 2.29 |
| CD.cDC1.CLEC9A.XCR1 | MPEG1 | 1.42 | CD.Fibro.ADAMDEC1.C7 | ANTXR1 | 0.58 | CD.Mstcl.HSPA1A.HSP90AA1 | MS4A2 | 2.23 |
| CD.cDC1.CLEC9A.XCR1 | IRF8 | 1.39 | CD.Fibro.ADAMDEC1.C7 | HTRA3 | 0.57 | CD.Mstcl.HSPA1A.HSP90AA1 | VWA5A | 2.13 |
| CD.cDC1.CLEC9A.XCR1 | PTPRE | 1.38 | CD.Fibro.ADAMDEC1.C7 | TMEM150C | 0.56 | CD.Mstcl.HSPA1A.HSP90AA1 | DUSP6 | 2.01 |
| CD.cDC1.CLEC9A.XCR1 | PLEK | 1.36 | CD.Fibro.ADAMDEC1.C7 | SCD5 | 0.56 | CD.Mstcl.HSPA1A.HSP90AA1 | AC020571.3 | 1.96 |
| CD.cDC1.CLEC9A.XCR1 | GOS2 | 1.30 | CD.Fibro.ADAMDEC1.C7 | GPX8 | 0.56 | CD.Mstcl.HSPA1A.HSP90AA1 | SLC18A2 | 1.80 |
| CD.cDC1.CLEC9A.XCR1 | CCND1 | 1.29 | CD.Fibro.ADAMDEC1.C7 | RUNX1T1 | 0.55 | CD.Mstcl.HSPA1A.HSP90AA1 | HDC | 1.65 |
| CD.cDC1.CLEC9A.XCR1 | NAAA | 1.28 | CD.Fibro.ADAMDEC1.C7 | PITX1 | 0.55 | CD.Mstcl.HSPA1A.HSP90AA1 | IL13 | 1.63 |
| CD.cDC1.CLEC9A.XCR1 | RGS10 | 1.27 | CD.Fibro.ADAMDEC1.C7 | ELANE | 0.54 | CD.Mstcl.HSPA1A.HSP90AA1 | IL1RL1 | 1.58 |
| CD.cDC1.CLEC9A.XCR1 | FNIP2 | 1.27 | CD.Fibro.ADAMDEC1.C7 | FGF13 | 0.47 | CD.Mstcl.HSPA1A.HSP90AA1 | KRT1 | 1.55 |
| CD.cDC1.CLEC9A.XCR1 | FGL2 | 1.24 | CD.Fibro.ADAMDEC1.C7 | TCEAL7 | 0.55 | CD.Mstcl.HSPA1A.HSP90AA1 | LTC4S | 1.47 |
| CD.cDC1.CLEC9A.XCR1 | BID | 1.23 | CD.Fibro.ADAMDEC1.C7 | FABP4 | 0.81 | CD.Mstcl.HSPA1A.HSP90AA1 | C1orf186 | 1.39 |
| CD.cDC1.CLEC9A.XCR1 | SPI1 | 1.21 | CD.Fibro.ADAMDEC1.C7 | ADAM28 | 1.36 | CD.Mstcl.HSPA1A.HSP90AA1 | KIT | 1.37 |
| CD.cDC1.CLEC9A.XCR1 | RAB32 | 1.21 | CD.Fibro.ADAMDEC1.C7 | FBLIM1 | 0.78 | CD.Mstcl.HSPA1A.HSP90AA1 | RAB27B | 1.31 |
| CD.cDC1.CLEC9A.XCR1 | CADM1 | 1.21 | CD.Fibro.ADAMDEC1.C7 | MDK | 0.98 | CD.Mstcl.HSPA1A.HSP90AA1 | RP11- | 1.16 |
| | | | | | | | 354E11.2 | |
| CD.cDC1.CLEC9A.XCR1 | ATG3 | 1.16 | CD.Fibro.ADAMDEC1.C7 | RBMS3 | 0.60 | CD.Mstcl.HSPA1A.HSP90AA1 | IL1RAPL1 | 1.14 |
| CD.cDC1.CLEC9A.XCR1 | SHTN1 | 1.14 | CD.Fibro.ADAMDEC1.C7 | RAB34 | 0.80 | CD.Mstcl.HSPA1A.HSP90AA1 | GCSAML | 0.90 |
| CD.cDC1.CLEC9A.XCR1 | BASP1 | 1.11 | CD.Fibro.ADAMDEC1.C7 | ANGPTL2 | 0.54 | CD.Mstcl.HSPA1A.HSP90AA1 | SVOPL | 0.84 |
| CD.cDC1.CLEC9A.XCR1 | KIAA0226L | 1.10 | CD.Fibro.ADAMDEC1.C7 | TCEAL4 | 0.97 | CD.Mstcl.HSPA1A.HSP90AA1 | TPSD1 | 0.76 |
| CD.cDC1.CLEC9A.XCR1 | CLNK | 1.09 | CD.Fibro.ADAMDEC1.C7 | F10 | 0.51 | CD.Mstcl.HSPA1A.HSP90AA1 | CMA1 | 1.36 |
| CD.cDC1.CLEC9A.XCR1 | CLEC7A | 1.07 | CD.Fibro.ADAMDEC1.C7 | C16orf89 | 0.54 | CD.Mstcl.HSPA1A.HSP90AA1 | HPGD | 1.84 |
| CD.cDC1.CLEC9A.XCR1 | CD40 | 1.07 | CD.Fibro.ADAMDEC1.C7 | PAMR1 | 0.44 | CD.Mstcl.JCHAIN.IGKC | TPSB2 | 3.59 |
| CD.cDC1.CLEC9A.XCR1 | ASB2 | 1.05 | CD.Fibro.ADAMDEC1.C7 | CST3 | 0.71 | CD.Mstcl.JCHAIN.IGKC | TPSAB1 | 3.06 |
| CD.cDC1.CLEC9A.XCR1 | XCR1 | 1.03 | CD.Fibro.ADAMDEC1.C7 | LINC01116 | 0.64 | CD.Mstcl.JCHAIN.IGKC | ANXA1 | 2.81 |
| CD.cDC1.CLEC9A.XCR1 | SAMHD1 | 1.00 | CD.Fibro.ADAMDEC1.C7 | RP11- | 0.80 | CD.Mstcl.JCHAIN.IGKC | HPGDS | 2.77 |
| | | | | 14N7.2 | | | | |
| CD.cDC1.CLEC9A.XCR1 | CTSZ | 0.98 | CD.Fibro.ADAMDEC1.C7 | C1QTNF2 | 0.47 | CD.Mstcl.JCHAIN.IGKC | CTSG | 2.46 |
| CD.cDC1.CLEC9A.XCR1 | AIF1 | 0.98 | CD.Fibro.ADAMDEC1.C7 | RCN1 | 0.98 | CD.Mstcl.JCHAIN.IGKC | CPA3 | 2.37 |
| CD.cDC1.CLEC9A.XCR1 | HLA-DMB | 0.96 | CD.Fibro.ADAMDEC1.C7 | LIFR | 0.59 | CD.Mstcl.JCHAIN.IGKC | AC020571.3 | 2.27 |
| CD.cDC1.CLEC9A.XCR1 | ENPP1 | 0.96 | CD.Fibro.ADAMDEC1.C7 | FBN1 | 0.60 | CD.Mstcl.JCHAIN.IGKC | MS4A2 | 2.19 |
| CD.cDC1.CLEC9A.XCR1 | BCL2A1 | 0.96 | CD.Fibro.ADAMDEC1.C7 | PTGFR | 0.49 | CD.Mstcl.JCHAIN.IGKC | HPGD | 2.18 |
| CD.cDC1.CLEC9A.XCR1 | SMCO4 | 0.95 | CD.Fibro.ADAMDEC1.C7 | CHL1 | 0.60 | CD.Mstcl.JCHAIN.IGKC | KRT1 | 1.92 |
| CD.cDC1.CLEC9A.XCR1 | SLAMF7 | 0.94 | CD.Fibro.ADAMDEC1.C7 | TWSG1 | 0.70 | CD.Mstcl.JCHAIN.IGKC | VWA5A | 1.88 |
| CD.cDC1.CLEC9A.XCR1 | RAB7B | 0.92 | CD.Fibro.ADAMDEC1.C7 | PLEKHA4 | 0.61 | CD.Mstcl.JCHAIN.IGKC | IL1RAPL1 | 1.56 |
| CD.cDC1.CLEC9A.XCR1 | FUCA1 | 0.90 | CD.Fibro.ADAMDEC1.C7 | RHOBTB3 | 0.84 | CD.Mstcl.JCHAIN.IGKC | CD9 | 2.18 |
| CD.cDC1.CLEC9A.XCR1 | SERPINB9 | 0.90 | CD.Fibro.ADAMDEC1.C7 | PTPRS | 0.83 | CD.Mstcl.JCHAIN.IGKC | RP11- | 1.46 |
| | | | | | | | 354E11.2 | |
| CD.cDC1.CLEC9A.XCR1 | PTMS | 0.87 | CD.Fibro.AGT.F3 | CXCL14 | 3.76 | CD.Mstcl.KLRB1.GNLY | TPSB2 | 3.24 |
| CD.cDC1.CLEC9A.XCR1 | VMO1 | 0.87 | CD.Fibro.AGT.F3 | CALD1 | 2.84 | CD.Mstcl.KLRB1.GNLY | TPSAB1 | 3.16 |
| CD.cDC1.CLEC9A.XCR1 | CXCL16 | 0.84 | CD.Fibro.AGT.F3 | C1R | 2.46 | CD.Mstcl.KLRB1.GNLY | CTSG | 2.68 |
| CD.cDC1.CLEC9A.XCR1 | C20orf27 | 0.84 | CD.Fibro.AGT.F3 | C1S | 2.45 | CD.Mstcl.TPSAB1.HPGDS | CPA3 | 1.31 |
| CD.cDC1.CLEC9A.XCR1 | MNDA | 0.84 | CD.Fibro.AGT.F3 | RARRES2 | 2.44 | CD.Mstcl.TPSAB1.HPGDS | CTSG | 1.63 |
| CD.cDC1.CLEC9A.XCR1 | PAK1 | 0.84 | CD.Fibro.AGT.F3 | EDNRB | 2.42 | CD.Mstcl.TPSAB1.HPGDS | HPGDS | 1.47 |
| CD.cDC1.CLEC9A.XCR1 | WDFY4 | 0.84 | CD.Fibro.AGT.F3 | F3 | 2.34 | CD.MyoFibro.MFAP5.SOSTDC1 | TAGLN | 4.14 |
| CD.cDC1.CLEC9A.XCR1 | BATF3 | 0.84 | CD.Fibro.AGT.F3 | TAGLN | 2.30 | CD.MyoFibro.MFAP5.SOSTDC1 | ACTA2 | 3.99 |
| CD.cDC1.CLEC9A.XCR1 | MIR4435-2HG | 0.83 | CD.Fibro.AGT.F3 | AGT | 2.29 | CD.MyoFibro.MFAP5.SOSTDC1 | ACTG2 | 3.77 |
| CD.cDC1.CLEC9A.XCR1 | DSE | 0.78 | CD.Fibro.AGT.F3 | IGFBP5 | 2.25 | CD.MyoFibro.MFAP5.SOSTDC1 | MYL9 | 3.37 |
| CD.cDC1.CLEC9A.XCR1 | CCDC88A | 0.78 | CD.Fibro.AGT.F3 | MMP2 | 2.22 | CD.MyoFibro.MFAP5.SOSTDC1 | TPM2 | 3.37 |
| CD.cDC1.CLEC9A.XCR1 | RP11- | 0.77 | CD.Fibro.AGT.F3 | COL6A2 | 2.21 | CD.MyoFibro.MFAP5.SOSTDC1 | MYLK | 2.93 |
| | 1143G9.4 | | | | | | | |
| CD.cDC1.CLEC9A.XCR1 | FAM26F | 0.76 | CD.Fibro.AGT.F3 | PDGFRA | 2.14 | CD.MyoFibro.MFAP5.SOSTDC1 | TPM1 | 2.92 |
| CD.cDC1.CLEC9A.XCR1 | HCK | 0.76 | CD.Fibro.AGT.F3 | TPM2 | 2.10 | CD.MyoFibro.MFAP5.SOSTDC1 | IGFBP5 | 2.84 |
| CD.cDC1.CLEC9A.XCR1 | CYB5R3 | 0.75 | CD.Fibro.AGT.F3 | FRZB | 1.89 | CD.MyoFibro.MFAP5.SOSTDC1 | MYH11 | 2.80 |
| CD.cDC1.CLEC9A.XCR1 | C1orf162 | 0.74 | CD.Fibro.AGT.F3 | TMEM119 | 1.63 | CD.MyoFibro.MFAP5.SOSTDC1 | CALD1 | 2.77 |
| CD.cDC1.CLEC9A.XCR1 | FAM110A | 0.72 | CD.Fibro.AGT.F3 | ENHO | 1.26 | CD.MyoFibro.MFAP5.SOSTDC1 | SOSTDC1 | 2.58 |
| CD.cDC1.CLEC9A.XCR1 | PLEKHO1 | 0.72 | CD.Fibro.AGT.F3 | PLAT | 2.17 | CD.MyoFibro.MFAP5.SOSTDC1 | DES | 2.36 |
| CD.cDC1.CLEC9A.XCR1 | SLAMF8 | 0.72 | CD.Fibro.AGT.F3 | ACTA2 | 2.52 | CD.MyoFibro.MFAP5.SOSTDC1 | RGS5 | 2.27 |
| CD.cDC1.CLEC9A.XCR1 | ASAP1 | 0.71 | CD.Fibro.AGT.F3 | COL3A1 | 2.22 | CD.MyoFibro.MFAP5.SOSTDC1 | FHL1 | 2.25 |
| CD.cDC1.CLEC9A.XCR1 | ZYX | 0.70 | CD.Fibro.AGT.F3 | ACTG2 | 1.52 | CD.MyoFibro.MFAP5.SOSTDC1 | HHIP | 2.24 |
| CD.cDC1.CLEC9A.XCR1 | P2RY14 | 0.70 | CD.Fibro.CA12.SFRP2 | MMP1 | 4.29 | CD.MyoFibro.MFAP5.SOSTDC1 | COL1A2 | 2.18 |
| CD.cDC1.CLEC9A.XCR1 | NET1 | 0.70 | CD.Fibro.CA12.SFRP2 | CHI3L1 | 3.80 | CD.MyoFibro.MFAP5.SOSTDC1 | LUM | 2.14 |
| CD.cDC1.CLEC9A.XCR1 | DENND1B | 0.69 | CD.Fibro.CA12.SFRP2 | COL1A1 | 3.02 | CD.MyoFibro.MFAP5.SOSTDC1 | COL3A1 | 2.09 |
| CD.cDC1.CLEC9A.XCR1 | KCNK6 | 0.69 | CD.Fibro.CA12.SFRP2 | CXCL13 | 2.96 | CD.MyoFibro.MFAP5.SOSTDC1 | FLNA | 2.04 |
| CD.cDC1.CLEC9A.XCR1 | HLA-DOB | 0.69 | CD.Fibro.CA12.SFRP2 | CTSK | 2.89 | CD.MyoFibro.MFAP5.SOSTDC1 | CNN1 | 2.04 |
| CD.cDC1.CLEC9A.XCR1 | AIM2 | 0.68 | CD.Fibro.CA12.SFRP2 | CXCL6 | 2.74 | CD.MyoFibro.MFAP5.SOSTDC1 | MFAP4 | 1.99 |
| CD.cDC1.CLEC9A.XCR1 | PRCP | 0.68 | CD.Fibro.CA12.SFRP2 | MMP2 | 2.58 | CD.MyoFibro.MFAP5.SOSTDC1 | SPARCL1 | 1.96 |
| CD.cDC1.CLEC9A.XCR1 | TMEM14A | 0.68 | CD.Fibro.CA12.SFRP2 | IGFBP5 | 2.51 | CD.MyoFibro.MFAP5.SOSTDC1 | CSRP1 | 1.94 |
| CD.cDC1.CLEC9A.XCR1 | RAB31 | 0.67 | CD.Fibro.CA12.SFRP2 | PCOLCE | 2.35 | CD.MyoFibro.MFAP5.SOSTDC1 | PRKCDBP | 1.94 |
| CD.cDC1.CLEC9A.XCR1 | LRRK2 | 0.67 | CD.Fibro.CA12.SFRP2 | CYR61 | 2.23 | CD.MyoFibro.MFAP5.SOSTDC1 | PDLIM3 | 1.92 |
| CD.cDC1.CLEC9A.XCR1 | VCAM1 | 0.66 | CD.Fibro.CA12.SFRP2 | FGF7 | 2.21 | CD.MyoFibro.MFAP5.SOSTDC1 | C1S | 1.91 |
| CD.cDC1.CLEC9A.XCR1 | LGALS9 | 0.65 | CD.Fibro.CA12.SFRP2 | FBLN1 | 2.18 | CD.MyoFibro.MFAP5.SOSTDC1 | SYNPO2 | 1.90 |
| CD.cDC1.CLEC9A.XCR1 | VAC14 | 0.65 | CD.Fibro.CA12.SFRP2 | NNMT | 2.16 | CD.MyoFibro.MFAP5.SOSTDC1 | PPP1R14A | 1.85 |
| CD.cDC1.CLEC9A.XCR1 | CAMK2D | 0.64 | CD.Fibro.CA12.SFRP2 | GREM1 | 2.15 | CD.MyoFibro.MFAP5.SOSTDC1 | CXCL14 | 1.85 |
| CD.cDC1.CLEC9A.XCR1 | SUSD3 | 0.63 | CD.Fibro.CA12.SFRP2 | THY1 | 2.03 | CD.MyoFibro.MFAP5.SOSTDC1 | NPNT | 1.84 |
| CD.cDC1.CLEC9A.XCR1 | QPCT | 0.63 | CD.Fibro.CA12.SFRP2 | SFRP2 | 2.02 | CD.MyoFibro.MFAP5.SOSTDC1 | COL1A1 | 1.84 |
| CD.cDC1.CLEC9A.XCR1 | SFT2D2 | 0.63 | CD.Fibro.CA12.SFRP2 | CTHRC1 | 2.00 | CD.MyoFibro.MFAP5.SOSTDC1 | SPARC | 1.83 |
| CD.cDC1.CLEC9A.XCR1 | CLIC2 | 0.62 | CD.Fibro.CA12.SFRP2 | TDO2 | 1.99 | CD.MyoFibro.MFAP5.SOSTDC1 | COL6A2 | 1.82 |
| CD.cDC1.CLEC9A.XCR1 | DAPP1 | 0.62 | CD.Fibro.CA12.SFRP2 | COL6A1 | 1.85 | CD.MyoFibro.MFAP5.SOSTDC1 | CAV1 | 1.80 |
| CD.cDC1.CLEC9A.XCR1 | PLCD1 | 0.61 | CD.Fibro.CA12.SFRP2 | AEBP1 | 1.72 | CD.MyoFibro.MFAP5.SOSTDC1 | CKB | 1.79 |
| CD.cDC1.CLEC9A.XCR1 | FLT3 | 0.61 | CD.Fibro.CA12.SFRP2 | CCDC80 | 1.71 | CD.MyoFibro.MFAP5.SOSTDC1 | TM4SF1 | 1.76 |
| CD.cDC1.CLEC9A.XCR1 | TACSTD2 | 0.61 | CD.Fibro.CA12.SFRP2 | FAP | 1.69 | CD.MyoFibro.MFAP5.SOSTDC1 | WFDC1 | 1.73 |
| CD.cDC1.CLEC9A.XCR1 | APOL3 | 0.60 | CD.Fibro.CA12.SFRP2 | BGN | 1.67 | CD.MyoFibro.MFAP5.SOSTDC1 | PDLIM7 | 1.70 |
| CD.cDC1.CLEC9A.XCR1 | C1orf21 | 0.59 | CD.Fibro.CA12.SFRP2 | COL6A3 | 1.61 | CD.MyoFibro.MFAP5.SOSTDC1 | DCN | 1.68 |
| CD.cDC1.CLEC9A.XCR1 | ACTN1 | 0.58 | CD.Fibro.CA12.SFRP2 | C3 | 1.53 | CD.MyoFibro.MFAP5.SOSTDC1 | LMOD1 | 1.67 |
| CD.cDC1.CLEC9A.XCR1 | MIR181A1HG | 0.58 | CD.Fibro.CA12.SFRP2 | EGFL6 | 1.52 | CD.MyoFibro.MFAP5.SOSTDC1 | NEXN | 1.62 |
| CD.cDC1.CLEC9A.XCR1 | HAVCR2 | 0.57 | CD.Fibro.CA12.SFRP2 | STEAP1 | 1.51 | CD.MyoFibro.MFAP5.SOSTDC1 | C1R | 1.60 |
| CD.cDC1.CLEC9A.XCR1 | LY86 | 0.57 | CD.Fibro.CA12.SFRP2 | PGF | 1.47 | CD.MyoFibro.MFAP5.SOSTDC1 | IGFBP7 | 1.60 |
| CD.cDC1.CLEC9A.XCR1 | TLR10 | 0.55 | CD.Fibro.CA12.SFRP2 | FBN1 | 1.43 | CD.MyoFibro.MFAP5.SOSTDC1 | CFH | 1.60 |
| CD.cDC1.CLEC9A.XCR1 | GSTM4 | 0.53 | CD.Fibro.CA12.SFRP2 | PDPN | 1.41 | CD.MyoFibro.MFAP5.SOSTDC1 | NUPR1 | 1.57 |
| CD.cDC1.CLEC9A.XCR1 | CPPED1 | 0.53 | CD.Fibro.CA12.SFRP2 | PDGFRA | 1.37 | CD.MyoFibro.MFAP5.SOSTDC1 | MMP2 | 1.56 |
| CD.cDC1.CLEC9A.XCR1 | CSF2RA | 0.52 | CD.Fibro.CA12.SFRP2 | WFDC2 | 1.36 | CD.MyoFibro.MFAP5.SOSTDC1 | TGFB111 | 1.56 |
| CD.cDC1.CLEC9A.XCR1 | PARVG | 0.51 | CD.Fibro.CA12.SFRP2 | LOXL1 | 1.33 | CD.MyoFibro.MFAP5.SOSTDC1 | CARMN | 1.51 |
| CD.cDC1.CLEC9A.XCR1 | SLC24A4 | 0.51 | CD.Fibro.CA12.SFRP2 | CA12 | 1.30 | CD.MyoFibro.MFAP5.SOSTDC1 | SMTN | 1.50 |
| CD.cDC1.CLEC9A.XCR1 | RFPL4A | 0.50 | CD.Fibro.CA12.SFRP2 | CFB | 1.27 | CD.MyoFibro.MFAP5.SOSTDC1 | PLN | 1.47 |
| CD.cDC1.CLEC9A.XCR1 | BTLA | 0.50 | CD.Fibro.CA12.SFRP2 | CP | 1.27 | CD.MyoFibro.MFAP5.SOSTDC1 | COL4A2 | 1.46 |
| CD.cDC1.CLEC9A.XCR1 | SLC46A3 | 0.49 | CD.Fibro.CA12.SFRP2 | MXRA5 | 1.25 | CD.MyoFibro.MFAP5.SOSTDC1 | RARRES2 | 1.42 |
| CD.cDC1.CLEC9A.XCR1 | TET2 | 0.49 | CD.Fibro.CA12.SFRP2 | EFEMP1 | 1.23 | CD.MyoFibro.MFAP5.SOSTDC1 | BCHE | 1.37 |
| CD.cDC1.CLEC9A.XCR1 | C10orf128 | 0.49 | CD.Fibro.CA12.SFRP2 | COL14A1 | 1.18 | CD.MyoFibro.MFAP5.SOSTDC1 | KCNMB1 | 1.36 |
| CD.cDC1.CLEC9A.XCR1 | FGD2 | 0.49 | CD.Fibro.CA12.SFRP2 | CYP7B1 | 1.12 | CD.MyoFibro.MFAP5.SOSTDC1 | MFAP5 | 1.35 |
| CD.cDC1.CLEC9A.XCR1 | HLA-DQB2 | 0.48 | CD.Fibro.CA12.SFRP2 | RSPO3 | 1.02 | CD.MyoFibro.MFAP5.SOSTDC1 | LINC01082 | 1.35 |
| CD.cDC1.CLEC9A.XCR1 | AOAH | 0.47 | CD.Fibro.CA12.SFRP2 | ANTXR1 | 1.02 | CD.MyoFibro.MFAP5.SOSTDC1 | HSD17B6 | 1.34 |
| CD.cDC1.CLEC9A.XCR1 | FKBP1B | 0.47 | CD.Fibro.CA12.SFRP2 | THBS2 | 1.00 | CD.MyoFibro.MFAP5.SOSTDC1 | NID1 | 1.33 |
| CD.cDC1.CLEC9A.XCR1 | SIGLEC10 | 0.46 | CD.Fibro.CA12.SFRP2 | CRABP2 | 0.99 | CD.MyoFibro.MFAP5.SOSTDC1 | FN1 | 1.31 |
| CD.cDC1.CLEC9A.XCR1 | REC8 | 0.45 | CD.Fibro.CA12.SFRP2 | COL12A1 | 0.97 | CD.MyoFibro.MFAP5.SOSTDC1 | LTBP1 | 1.30 |
| CD.cDC1.CLEC9A.XCR1 | MYCL | 0.45 | CD.Fibro.CA12.SFRP2 | PRRX1 | 0.97 | CD.MyoFibro.MFAP5.SOSTDC1 | EDNRB | 1.28 |
| CD.cDC1.CLEC9A.XCR1 | DBN1 | 0.45 | CD.Fibro.CA12.SFRP2 | PTGES | 0.96 | CD.MyoFibro.MFAP5.SOSTDC1 | RBPMS | 1.28 |
| CD.cDC1.CLEC9A.XCR1 | P2RY6 | 0.44 | CD.Fibro.CA12.SFRP2 | AKR1C1 | 0.96 | CD.MyoFibro.MFAP5.SOSTDC1 | COL6A1 | 1.27 |
| CD.cDC1.CLEC9A.XCR1 | GCSAM | 0.43 | CD.Fibro.CA12.SFRP2 | GAS1 | 0.93 | CD.MyoFibro.MFAP5.SOSTDC1 | PTCH1 | 1.23 |
| CD.cDC1.CLEC9A.XCR1 | SERPINF2 | 0.43 | CD.Fibro.CA12.SFRP2 | BICC1 | 0.93 | CD.MyoFibro.MFAP5.SOSTDC1 | MAP1B | 1.20 |
| CD.cDC1.CLEC9A.XCR1 | SH2B3 | 0.43 | CD.Fibro.CA12.SFRP2 | LTBP1 | 0.90 | CD.MyoFibro.MFAP5.SOSTDC1 | MYOCD | 1.18 |
| CD.cDC1.CLEC9A.XCR1 | APOBR | 0.43 | CD.Fibro.CA12.SFRP2 | FAM20A | 0.87 | CD.MyoFibro.MFAP5.SOSTDC1 | COL4A1 | 1.18 |
| CD.cDC1.CLEC9A.XCR1 | HDAC9 | 0.42 | CD.Fibro.CA12.SFRP2 | TWIST2 | 0.84 | CD.MyoFibro.MFAP5.SOSTDC1 | DMD | 1.11 |
| CD.cDC1.CLEC9A.XCR1 | GNA15 | 0.42 | CD.Fibro.CA12.SFRP2 | ANGPTL2 | 0.82 | CD.MyoFibro.MFAP5.SOSTDC1 | CES1 | 1.11 |
| CD.cDC1.CLEC9A.XCR1 | NAV1 | 0.42 | CD.Fibro.CA12.SFRP2 | IGF2 | 0.79 | CD.MyoFibro.MFAP5.SOSTDC1 | WNT2B | 1.08 |
| CD.cDC1.CLEC9A.XCR1 | PNMA1 | 0.41 | CD.Fibro.CA12.SFRP2 | CPXM1 | 0.75 | CD.MyoFibro.MFAP5.SOSTDC1 | MSRB3 | 1.03 |
| CD.cDC1.CLEC9A.XCR1 | SCARF1 | 0.40 | CD.Fibro.CA12.SFRP2 | MASP1 | 0.72 | CD.MyoFibro.MFAP5.SOSTDC1 | CRYAB | 1.01 |
| CD.cDC1.CLEC9A.XCR1 | MCOLN2 | 0.40 | CD.Fibro.CA12.SFRP2 | CEMIP | 0.71 | CD.MyoFibro.MFAP5.SOSTDC1 | PCDH7 | 0.96 |
| CD.cDC1.CLEC9A.XCR1 | FSCN1 | 0.38 | CD.Fibro.CA12.SFRP2 | BHLHE22 | 0.70 | CD.MyoFibro.MFAP5.SOSTDC1 | RP11- | 0.95 |
| | | | | | | | 39404.5 | |
| CD.cDC1.CLEC9A.XCR1 | CCSER1 | 0.37 | CD.Fibro.CA12.SFRP2 | COL8A1 | 0.66 | CD.MyoFibro.MFAP5.SOSTDC1 | HHIP-AS1 | 0.89 |
| CD.cDC1.CLEC9A.XCR1 | ADAP2 | 0.37 | CD.Fibro.CA12.SFRP2 | NRCAM | 0.44 | CD.MyoFibro.MFAP5.SOSTDC1 | MRGPRF | 0.87 |
| CD.cDC1.CLEC9A.XCR1 | DGAT2 | 0.34 | CD.Fibro.CA12.SFRP2 | AOX1 | 0.42 | CD.MyoFibro.MFAP5.SOSTDC1 | SYT10 | 0.77 |
| CD.cDC1.CLEC9A.XCR1 | RP3-522D1.1 | 0.32 | CD.Fibro.CA12.SFRP2 | HTR2A | 0.41 | CD.MyoFibro.MFAP5.SOSTDC1 | PPP1R12B | 1.39 |
| CD.cDC1.CLEC9A.XCR1 | TNNI2 | 0.32 | CD.Fibro.CA12.SFRP2 | MMP3 | 3.41 | CD.MyoFibro.MFAP5.SOSTDC1 | COL6A3 | 1.27 |
| CD.cDC1.CLEC9A.XCR1 | ZNF366 | 0.28 | CD.Fibro.CA12.SFRP2 | PODNL1 | 0.39 | CD.MyoFibro.MFAP5.SOSTDC1 | C9orf3 | 1.16 |
| CD.cDC1.CLEC9A.XCR1 | CCDC126 | 0.37 | CD.Fibro.CA12.SFRP2 | COL3A1 | 3.21 | CD.MyoFibro.MFAP5.SOSTDC1 | CDH6 | 0.74 |
| CD.cDC1.CLEC9A.XCR1 | HLA-DPA1 | 2.13 | CD.Fibro.CA12.SFRP2 | CLMP | 1.03 | CD.MyoFibro.MFAP5.SOSTDC1 | FBLN1 | 1.23 |
| CD.cDC1.CLEC9A.XCR1 | ACER3 | 0.43 | CD.Fibro.CA12.SFRP2 | DCN | 3.64 | CD.MyoFibro.MFAP5.SOSTDC1 | LPP | 2.07 |
| CD.cDC1.CLEC9A.XCR1 | AP1S2 | 0.73 | CD.Fibro.CA12.SFRP2 | MEG3 | 1.21 | CD.MyoFibro.MFAP5.SOSTDC1 | ADAMDEC1 | 1.96 |
| CD.cDC1.CLEC9A.XCR1 | HPS5 | 0.40 | CD.Fibro.CA12.SFRP2 | MFAP2 | 0.75 | CD.NK.CCL3.CD160 | NKG7 | 2.75 |
| CD.cDC1.CLEC9A.XCR1 | TGFBI | 0.52 | CD.Fibro.CA12.SFRP2 | FKBP10 | 0.90 | CD.NK.CCL3.CD160 | GZMA | 2.61 |
| CD.cDC1.CLEC9A.XCR1 | TPMT | 0.52 | CD.Fibro.CA12.SFRP2 | CXCL12 | 2.69 | CD.NK.CCL3.CD160 | CCL4 | 2.42 |
| CD.cDC1.CLEC9A.XCR1 | SLC31A2 | 0.51 | CD.Fibro.CA12.SFRP2 | FMOD | 0.73 | CD.NK.CCL3.CD160 | CMC1 | 2.34 |
| CD.cDC1.CLEC9A.XCR1 | NDRG2 | 0.50 | CD.Fibro.CA12.SFRP2 | RCN3 | 0.84 | CD.NK.CCL3.CD160 | KLRD1 | 2.30 |
| CD.cDC1.CLEC9A.XCR1 | RAB29 | 0.45 | CD.Fibro.CCL13.CXCL6 | CCL13 | 4.31 | CD.NK.CCL3.CD160 | TRDC | 2.29 |
| CD.cDC1.CLEC9A.XCR1 | ROGDI | 0.45 | CD.Fibro.CCL13.CXCL6 | CCL11 | 4.03 | CD.NK.CCL3.CD160 | KLRF1 | 2.02 |
| CD.cDC1.CLEC9A.XCR1 | HLA-DMA | 1.29 | CD.Fibro.CCL13.CXCL6 | LUM | 3.76 | CD.NK.CCL3.CD160 | CCL5 | 1.91 |
| CD.cDC1.CLEC9A.XCR1 | GMZA | 0.44 | CD.Fibro.CCL13.CXCL6 | CXCL14 | 3.43 | CD.NK.CCL3.CD160 | GZMB | 1.87 |
| CD.cDC1.CLEC9A.XCR1 | PPIF | 0.73 | CD.Fibro.CCL13.CXCL6 | DCN | 3.31 | CD.NK.CCL3.CD160 | XCL1 | 1.78 |
| CD.cDC1.CLEC9A.XCR1 | FAM129A | 0.40 | CD.Fibro.CCL13.CXCL6 | CXCL1 | 3.29 | CD.NK.CCL3.CD160 | CD160 | 1.78 |
| CD.cDC1.CLEC9A.XCR1 | GPR137B | 0.51 | CD.Fibro.CCL13.CXCL6 | ADAMDEC | 3.21 | CD.NK.CCL3.CD160 | XCL2 | 1.77 |
| CD.cDC1.CLEC9A.XCR1 | CD86 | 0.50 | CD.Fibro.CCL13.CXCL6 | CXCL6 | 3.16 | CD.NK.CCL3.CD160 | IL2RB | 1.76 |
| CD.cDC1.CLEC9A.XCR1 | TAP1 | 1.00 | CD.Fibro.CCL13.CXCL6 | CFD | 3.05 | CD.NK.CCL3.CD160 | CCL3 | 1.73 |
| CD.cDC1.CLEC9A.XCR1 | DUSP10 | 0.51 | CD.Fibro.CCL13.CXCL6 | RARRES2 | 3.02 | CD.NK.CCL3.CD160 | AREG | 1.73 |
| CD.cDC1.CLEC9A.XCR1 | HLA-DRB1 | 2.01 | CD.Fibro.CCL13.CXCL6 | CCL8 | 2.90 | CD.NK.CCL3.CD160 | KLRB1 | 1.67 |
| CD.cDC1.CLEC9A.XCR1 | LSP1 | 1.39 | CD.Fibro.CCL13.CXCL6 | CALD1 | 2.85 | CD.NK.CCL3.CD160 | CLIC3 | 1.65 |
| CD.cDC1.CLEC9A.XCR1 | BAG3 | 0.71 | CD.Fibro.CCL13.CXCL6 | CCL2 | 2.72 | CD.NK.CCL3.CD160 | CST7 | 1.61 |
| CD.cDC1.CLEC9A.XCR1 | SPINT2 | 0.84 | CD.Fibro.CCL13.CXCL6 | COL3A1 | 2.62 | CD.NK.CCL3.CD160 | KLRC1 | 1.60 |
| CD.cDC1.CLEC9A.XCR1 | PIK3CB | 0.43 | CD.Fibro.CCL13.CXCL6 | IGFBP7 | 2.60 | CD.NK.CCL3.CD160 | FCER1G | 1.58 |
| CD.cDC1.CLEC9A.XCR1 | SCML1 | 0.50 | CD.Fibro.CCL13.CXCL6 | PTGDS | 2.53 | CD.NK.CCL3.CD160 | GZMK | 1.56 |
| CD.cDC2.CD1C.AREG | MNDA | 2.00 | CD.Fibro.CCL13.CXCL6 | MFAP4 | 2.44 | CD.NK.CCL3.CD160 | CTSW | 1.51 |
| CD.cDC2.CD1C.AREG | CLEC10A | 1.77 | CD.Fibro.CCL13.CXCL6 | COL1A2 | 2.42 | CD.NK.CCL3.CD160 | CD7 | 1.49 |
| CD.cDC2.CD1C.AREG | MS4A6A | 1.74 | CD.Fibro.CCL13.CXCL6 | ADH1B | 2.41 | CD.NK.CCL3.CD160 | B3GNT7 | 1.42 |
| CD.cDC2.CD1C.AREG | IDO1 | 1.70 | CD.Fibro.CCL13.CXCL6 | PLPP1 | 2.37 | CD.NK.CCL3.CD160 | IFITM2 | 1.41 |
| CD.cDC2.CD1C.AREG | IFI30 | 1.68 | CD.Fibro.CCL13.CXCL6 | C1S | 2.37 | CD.NK.CCL3.CD160 | PRF1 | 1.40 |
| CD.cDC2.CD1C.AREG | CPVL | 1.62 | CD.Fibro.CCL13.CXCL6 | APOE | 2.34 | CD.NK.CCL3.CD160 | HOPX | 1.40 |
| CD.cDC2.CD1C.AREG | IGSF6 | 1.47 | CD.Fibro.CCL13.CXCL6 | CFH | 2.34 | CD.NK.CCL3.CD160 | PIK3R1 | 1.37 |
| CD.cDC2.CD1C.AREG | CD86 | 1.37 | CD.Fibro.CCL13.CXCL6 | TFPI | 2.28 | CD.NK.CCL3.CD160 | TYROBP | 1.35 |
| CD.cDC2.CD1C.AREG | CD1E | 1.23 | CD.Fibro.CCL13.CXCL6 | SERPINF1 | 2.20 | CD.NK.CCL3.CD160 | NR4A2 | 1.33 |
| CD.cDC2.CD1C.AREG | CFP | 1.22 | CD.Fibro.CCL13.CXCL6 | A2M | 2.19 | CD.NK.CCL3.CD160 | GZMM | 1.31 |
| CD.cDC2.CD1C.AREG | CLEC4A | 1.02 | CD.Fibro.CCL13.CXCL6 | SFRP1 | 2.18 | CD.NK.CCL3.CD160 | ALOX5AP | 1.29 |
| CD.cDC2.CD1C.AREG | P2RY13 | 0.96 | CD.Fibro.CCL13.CXCL6 | C1R | 2.12 | CD.NK.CCL3.CD160 | MATK | 1.27 |
| CD.cDC2.CD1C.AREG | CD207 | 0.80 | CD.Fibro.CCL13.CXCL6 | IGFBP5 | 2.11 | CD.NK.CCL3.CD160 | HCST | 1.25 |
| CD.cDC2.CD1C.AREG | MRC1 | 0.95 | CD.Fibro.CCL13.CXCL6 | GNG11 | 2.03 | CD.NK.CCL3.CD160 | SRGN | 1.15 |
| CD.cDC2.CD1C.AREG | JAML | 1.84 | CD.Fibro.CCL13.CXCL6 | VCAN | 2.00 | CD.NK.CCL3.CD160 | SH2D1A | 1.13 |
| CD.cDC2.CD1C.AREG | FAM110A | 1.07 | CD.Fibro.CCL13.CXCL6 | FBLN1 | 1.95 | CD.NK.CCL3.CD160 | TMIGD2 | 1.10 |
| CD.cDC2.CD1C.AREG | CACNA2D3 | 0.50 | CD.Fibro.CCL13.CXCL6 | QSOX1 | 1.93 | CD.NK.CCL3.CD160 | KRT86 | 0.99 |
| CD.cDC2.CD1C.AREG | CD1C | 1.27 | CD.Fibro.CCL13.CXCL6 | SPARCL1 | 1.84 | CD.NK.CCL3.CD160 | FASLG | 0.78 |
| CD.cDC2.CD1C.CD207 | FCER1A | 1.63 | CD.Fibro.CCL13.CXCL6 | CTSK | 1.78 | CD.NK.GNLY.FCER1G | GNLY | 3.97 |
| CD.cDC2.CD1C.CD207 | CD207 | 1.18 | CD.Fibro.CCL13.CXCL6 | TCF21 | 1.72 | CD.NK.GNLY.FCER1G | GZMA | 3.06 |
| CD.cDC2.CD1C.CD207 | CACNA2D3 | 0.62 | CD.Fibro.CCL13.CXCL6 | SPARC | 1.71 | CD.NK.GNLY.FCER1G | GZMB | 2.81 |
| CD.cDC2.CD1C.CD207 | ITGAX | 0.99 | CD.Fibro.CCL13.CXCL6 | PPP1R14A | 1.71 | CD.NK.GNLY.FCER1G | NKG7 | 2.69 |
| CD.cDC2.CD1C.NDRG2 | CLEC10A | 2.04 | CD.Fibro.CCL13.CXCL6 | RBP1 | 1.70 | CD.NK.GNLY.FCER1G | CMC1 | 2.54 |
| CD.cDC2.CD1C.NDRG2 | FCER1A | 1.71 | CD.Fibro.CCL13.CXCL6 | TFPI2 | 1.69 | CD.NK.GNLY.FCER1G | KLRD1 | 2.44 |
| CD.cDC2.CD1C.NDRG2 | IFI30 | 1.57 | CD.Fibro.CCL13.CXCL6 | GPX3 | 1.64 | CD.NK.GNLY.FCER1G | TRDC | 2.38 |
| CD.cDC2.CD1C.NDRG2 | CD1C | 1.54 | CD.Fibro.CCL13.CXCL6 | PLTP | 1.62 | CD.NK.GNLY.FCER1G | KLRF1 | 2.37 |
| CD.cDC2.CD1C.NDRG2 | MNDA | 1.39 | CD.Fibro.CCL13.CXCL6 | CRYBG3 | 1.57 | CD.NK.GNLY.FCER1G | KLRC1 | 2.21 |
| CD.cDC2.CD1C.NDRG2 | CFP | 1.31 | CD.Fibro.CCL13.CXCL6 | MMP2 | 1.57 | CD.NK.GNLY.FCER1G | IL2RB | 1.81 |
| CD.cDC2.CD1C.NDRG2 | IGSF6 | 1.18 | CD.Fibro.CCL13.CXCL6 | COL18A1 | 1.56 | CD.NK.GNLY.FCER1G | GZMK | 1.81 |
| CD.cDC2.CD1C.NDRG2 | CD1E | 1.04 | CD.Fibro.CCL13.CXCL6 | FHL2 | 1.53 | CD.NK.GNLY.FCER1G | GZMH | 1.51 |
| CD.cDC2.CD1C.NDRG2 | CD207 | 0.99 | CD.Fibro.CCL13.CXCL6 | EDIL3 | 1.53 | CD.NK.GNLY.FCER1G | PRF1 | 1.63 |
| CD.cDC2.CD1C.NDRG2 | PLD4 | 0.98 | CD.Fibro.CCL13.CXCL6 | NID1 | 1.52 | CD.NK.GNLY.FCGR3A | FGFBP2 | 1.84 |
| CD.cDC2.CD1C.NDRG2 | CLEC4A | 0.87 | CD.Fibro.CCL13.CXCL6 | COL1A1 | 1.51 | CD.NK.GNLY.GZMB | GNLY | 3.52 |
| CD.cDC2.CD1C.NDRG2 | CSF2RA | 0.80 | CD.Fibro.CCL13.CXCL6 | TPM2 | 1.50 | CD.NK.GNLY.GZMB | GZMB | 3.34 |
| CD.cDC2.CD1C.NDRG2 | FGR | 0.73 | CD.Fibro.CCL13.CXCL6 | PCDH18 | 1.48 | CD.NK.GNLY.GZMB | FGFBP2 | 2.54 |
| CD.cDC2.CD1C.NDRG2 | CACNA2D3 | 0.67 | CD.Fibro.CCL13.CXCL6 | COL14A1 | 1.43 | CD.NK.GNLY.GZMB | PRF1 | 2.75 |
| CD.cDC2.CD1C.NDRG2 | CD33 | 0.61 | CD.Fibro.CCL13.CXCL6 | PDGFRA | 1.42 | CD.NK.GNLY.IFNG | GNLY | 3.56 |
| CD.cDC2.CD1C.NDRG2 | CPVL | 1.71 | CD.Fibro.CCL13.CXCL6 | FIBIN | 1.40 | CD.NK.GNLY.IFNG | KLRD1 | 2.22 |
| CD.cDC2.CLEC10A.CD1E | CD1E | 2.09 | CD.Fibro.CCL13.CXCL6 | PRKCDBP | 1.39 | CD.NK.GNLY.IFNG | KLRF1 | 2.11 |
| CD.cDC2.CLEC10A.CD1E | CD1B | 0.67 | CD.Fibro.CCL13.CXCL6 | COL6A3 | 1.38 | CD.NK.GNLY.IFNG | XCL1 | 2.04 |
| CD.cDC2.CLEC10A.CD1E | CD207 | 0.82 | CD.Fibro.CCL13.CXCL6 | BMP5 | 1.37 | CD.NK.GNLY.IFNG | KLRC1 | 2.04 |
| CD.cDC2.CLEC10A.FCGR2B | CLEC10A | 2.27 | CD.Fibro.CCL13.CXCL6 | SFTA1P | 1.35 | CD.NK.GNLY.IFNG | TRDC | 1.93 |
| CD.cDC2.CLEC10A.FCGR2B | FCER1A | 1.65 | CD.Fibro.CCL13.CXCL6 | CP | 1.34 | CD.NK.GNLY.IFNG | XCL2 | 1.81 |
| CD.cDC2.FCER1A.CD1C | CLEC10A | 2.20 | CD.Fibro.CCL13.CXCL6 | FBN1 | 1.34 | CD.NK.GNLY.IFNG | IFNG | 2.58 |
| CD.cDC2.FCER1A.CD1C | FCER1A | 1.99 | CD.Fibro.CCL13.CXCL6 | DDR2 | 1.33 | CD.NK.GNLY.IFNG | GZMB | 2.60 |
| CD.cDC2.FCER1A.CD1C | CD1C | 1.77 | CD.Fibro.CCL13.CXCL6 | LINC01082 | 1.31 | CD.NK.GNLY.KLRC1 | GNLY | 3.75 |
| CD.cDC2.FCER1A.CD1C | CD1E | 1.36 | CD.Fibro.CCL13.CXCL6 | FSTL1 | 1.28 | CD.NK.GNLY.KLRC1 | CTSW | 2.48 |
| CD.cDC2.FCER1A.CD1C | CD207 | 1.14 | CD.Fibro.CCL13.CXCL6 | COL5A2 | 1.28 | CD.NK.GNLY.KLRC1 | KLRC1 | 2.35 |
| CD.cDC2.FCER1A.CD1C | CACNA2D3 | 0.61 | CD.Fibro.CCL13.CXCL6 | MEG3 | 1.27 | CD.NK.GNLY.KLRC1 | KLRD1 | 2.33 |
| CD.cDC2.MKI67.CD1C | FCER1A | 2.16 | CD.Fibro.CCL13.CXCL6 | PLAC9 | 1.26 | CD.NK.GNLY.KLRC1 | KLRF1 | 2.12 |
| CD.cDC2.RALA.CD1C | CD207 | 0.90 | CD.Fibro.CCL13.CXCL6 | OLFML3 | 1.23 | CD.NK.GNLY.KLRC1 | TRDC | 2.03 |
| CD.cDC2.RALA.CD1C | NRIP3 | 0.52 | CD.Fibro.CCL13.CXCL6 | EDNRB | 1.22 | CD.NK.GNLY.KLRC1 | NKG7 | 1.92 |
| CD.cDC2.RALA.CD1C | CLEC10A | 1.79 | CD.Fibro.CCL13.CXCL6 | HAPLN1 | 1.21 | CD.NK.GNLY.KLRC1 | XCL2 | 1.87 |
| CD.DC.CCR7.FSCN1 | MARCKSL1 | 2.69 | CD.Fibro.CCL13.CXCL6 | BMP4 | 1.21 | CD.NK.GNLY.KLRC1 | XCL1 | 1.86 |
| CD.DC.CCR7.FSCN1 | IDO1 | 2.56 | CD.Fibro.CCL13.CXCL6 | EFEMP1 | 1.18 | CD.NK.GNLY.KLRC1 | GZMB | 2.01 |
| CD.DC.CCR7.FSCN1 | CCL19 | 2.53 | CD.Fibro.CCL13.CXCL6 | THY1 | 1.17 | CD.NK.GNLY.KLRC1 | SPTSSB | 0.80 |
| CD.DC.CCR7.FSCN1 | FSCN1 | 2.48 | CD.Fibro.CCL13.CXCL6 | ANGPTL1 | 1.15 | CD.NK.GNLY.KLRC1 | HOPX | 1.79 |
| CD.DC.CCR7.FSCN1 | LAMP3 | 1.86 | CD.Fibro.CCL13.CXCL6 | PCDH7 | 1.14 | CD.NK.GNLY.XCL1 | GNLY | 3.67 |
| CD.DC.CCR7.FSCN1 | CCL22 | 1.66 | CD.Fibro.CCL13.CXCL6 | SULF1 | 1.12 | CD.NK.GNLY.XCL1 | TRDC | 2.48 |
| CD.DC.CCR7.FSCN1 | CD1E | 1.51 | CD.Fibro.CCL13.CXCL6 | HGF | 1.12 | CD.NK.GNLY.XCL1 | KLRD1 | 2.05 |
| CD.DC.CCR7.FSCN1 | EBI3 | 1.50 | CD.Fibro.CCL13.CXCL6 | GPX8 | 1.08 | CD.NK.GNLY.XCL1 | XCL1 | 1.95 |
| CD.DC.CCR7.FSCN1 | CSF2RA | 1.47 | CD.Fibro.CCL13.CXCL6 | FHL1 | 1.52 | CD.NK.GNLY.XCL1 | KLRF1 | 1.78 |
| CD.DC.CCR7.FSCN1 | CLIC2 | 1.30 | CD.Fibro.CCL13.CXCL6 | NGFRAP1 | 1.84 | CD.NK.GNLY.XCL1 | XCL2 | 1.94 |
| CD.DC.CCR7.FSCN1 | RASSF4 | 1.20 | CD.Fibro.CCL13.CXCL6 | FGF7 | 1.14 | CD.NK.MKI67.GZMA | TRDC | 2.11 |
| CD.DC.CCR7.FSCN1 | HMSD | 0.74 | CD.Fibro.CCL13.CXCL6 | MYL9 | 1.30 | CD.NK.MKI67.GZMA | MKI67 | 2.04 |
| CD.DC.CCR7.FSCN1 | TVP23A | 0.62 | CD.Fibro.CCL13.CXCL6 | PMP22 | 1.57 | CD.NK.MKI67.GZMA | KLRD1 | 2.03 |
| CD.DC.CXCL10.IDO1 | CD1E | 1.43 | CD.Fibro.CCL19.IRF7 | FBLN1 | 2.45 | CD.NK.MKI67.GZMA | TOP2A | 2.01 |
| CD.DC.LTB.IL22RA2 | IL22RA2 | 1.62 | CD.Fibro.CCL19.IRF7 | LINC01082 | 1.45 | CD.NK.MKI67.GZMA | UBE2C | 1.95 |
| CD.DC.LTB.IL22RA2 | CFP | 1.49 | CD.Fibro.CCL21.CCL19 | CCL21 | 5.26 | CD.NK.MKI67.GZMA | CENPF | 1.90 |
| CD.DC.LTB.IL22RA2 | PSTPIP2 | 0.97 | CD.Fibro.CCL21.CCL19 | CCL19 | 4.99 | CD.NK.MKI67.GZMA | ASPM | 1.79 |
| CD.DC.LTB.IL22RA2 | FLT3 | 0.64 | CD.Fibro.CCL21.CCL19 | PTGDS | 2.97 | CD.NK.MKI67.GZMA | NUSAP1 | 1.76 |
| CD.DC.LTB.IL22RA2 | LINC01531 | 0.56 | CD.Fibro.CCL21.CCL19 | C3 | 2.87 | CD.NK.MKI67.GZMA | TYMS | 1.68 |
| CD.DC.LTB.IL22RA2 | SFTPD | 0.47 | CD.Fibro.CCL21.CCL19 | DCN | 2.54 | CD.NK.MKI67.GZMA | KLRF1 | 1.52 |
| CD.DC.LTB.IL22RA2 | SYT2 | 0.35 | CD.Fibro.CCL21.CCL19 | C15 | 2.39 | CD.NK.MKI67.GZMA | AURKB | 1.34 |
| CD.DC.LTB.IL22RA2 | CD300LG | 0.32 | CD.Fibro.CCL21.CCL19 | LUM | 2.34 | CD.NK.MKI67.GZMA | XCL2 | 1.70 |
| CD.DC.LTB.IL22RA2 | PKIB | 1.08 | CD.Fibro.CCL21.CCL19 | TDO2 | 1.91 | CD.NK.MKI67.GZMA | KLRC1 | 1.61 |
| CD.DC.LTB.IL22RA2 | PIK3R6 | 0.46 | CD.Fibro.CCL21.CCL19 | DPT | 1.63 | CD.NK.MKI67.GZMA | XCL1 | 1.39 |
| CD.EC.ADH1C.EDN1 | OLFM4 | 2.65 | CD.Fibro.CCL21.CCL19 | GREM1 | 1.58 | CD.NK.MKI67.GZMA | CDK1 | 1.57 |
| CD.EC.ADH1C.EDN1 | TSPAN8 | 2.21 | CD.Fibro.CCL21.CCL19 | FGF7 | 1.54 | CD.NK.MKI67.GZMA | KIF11 | 0.98 |
| CD.EC.ADH1C.EDN1 | SPINK1 | 2.17 | CD.Fibro.CCL21.CCL19 | CCDC80 | 1.53 | CD.NK.MKI67.GZMA | EOMES | 0.80 |
| CD.EC.ADH1C.EDN1 | PIGR | 2.16 | CD.Fibro.CCL21.CCL19 | RSPO3 | 1.18 | CD.NK.MKI67.GZMA | KIF23 | 0.94 |
| CD.EC.ADH1C.EDN1 | MT1E | 2.14 | CD.Fibro.CCL21.CCL19 | SHISA3 | 0.93 | CD.Paneth.DEFA6.ITLN2 | DEFA5 | 6.47 |
| CD.EC.ADH1C.EDN1 | MT1G | 2.00 | CD.Fibro.CCL21.CCL19 | CHRDL2 | 0.66 | CD.Paneth.DEFA6.ITLN2 | REG3A | 5.91 |
| CD.EC.ADH1C.EDN1 | EPCAM | 1.92 | CD.Fibro.CCL21.CCL19 | PRELP | 0.88 | CD.Paneth.DEFA6.ITLN2 | DEFA6 | 5.88 |
| CD.EC.ADH1C.EDN1 | LGALS4 | 1.91 | CD.Fibro.CCL21.CCL19 | ABI3BP | 1.29 | CD.Paneth.DEFA6.ITLN2 | PRSS2 | 5.28 |
| CD.EC.ADH1C.EDN1 | EDN1 | 1.67 | CD.Fibro.CCL21.CCL19 | RARRES2 | 2.29 | CD.Paneth.DEFA6.ITLN2 | PLA2G2A | 4.69 |
| CD.EC.ADH1C.EDN1 | DMBT1 | 1.67 | CD.Fibro.CCL21.CCL19 | C1R | 2.18 | CD.Paneth.DEFA6.ITLN2 | ITLN2 | 3.59 |
| CD.EC.ADH1C.EDN1 | CCL25 | 1.65 | CD.Fibro.CCL21.CCL19 | VCAM1 | 1.83 | CD.Paneth.DEFA6.ITLN2 | LYZ | 3.22 |
| CD.EC.ADH1C.EDN1 | CLDN3 | 1.58 | CD.Fibro.CD24.IGHG4 | CXCL6 | 3.16 | CD.Paneth.DEFA6.ITLN2 | GUCAZA | 3.09 |
| CD.EC.ADH1C.EDN1 | GSTA1 | 1.56 | CD.Fibro.CD24.IGHG4 | ADAMTS5 | 1.40 | CD.Paneth.DEFA6.ITLN2 | SERPINA1 | 2.23 |
| CD.EC.ADH1C.EDN1 | CLDN4 | 1.53 | CD.Fibro.CD24.IGHG4 | CP | 2.00 | CD.Paneth.DEFA6.ITLN2 | LCN2 | 2.08 |
| CD.EC.ADH1C.EDN1 | GPX2 | 1.52 | CD.Fibro.CHI3L1.CXCL3 | CXCL6 | 3.31 | CD.Paneth.DEFA6.ITLN2 | REG1A | 1.93 |
| CD.EC.ADH1C.EDN1 | SLC12A2 | 1.51 | CD.Fibro.CHI3L1.CXCL3 | CHI3L1 | 2.69 | CD.Paneth.DEFA6.ITLN2 | REG1B | 1.84 |
| CD.EC.ADH1C.EDN1 | MT1F | 1.42 | CD.Fibro.CHI3L1.CXCL3 | CTSK | 2.24 | CD.Paneth.DEFA6.ITLN2 | TFF3 | 1.67 |
| CD.EC.ADH1C.EDN1 | ADH1C | 1.42 | CD.Fibro.CHI3L1.CXCL3 | PCOLCE | 1.81 | CD.Paneth.DEFA6.ITLN2 | RP11- | 1.17 |
| | | | | | | | 1143G9.4 | |
| CD.EC.ADH1C.EDN1 | KRT19 | 1.42 | CD.Fibro.CHI3L1.CXCL3 | TDO2 | 1.59 | CD.Paneth.DEFA6.ITLN2 | IGFBP2 | 1.02 |
| CD.EC.ADH1C.EDN1 | C19orf33 | 1.33 | CD.Fibro.CHI3L1.CXCL3 | PDPN | 1.30 | CD.Paneth.DEFA6.ITLN2 | HES6 | 1.01 |
| CD.EC.ADH1C.EDN1 | MT1H | 1.29 | CD.Fibro.CHI3L1.CXCL3 | MMP2 | 1.95 | CD.Paneth.DEFA6.ITLN2 | GPX2 | 0.99 |
| CD.EC.ADH1C.EDN1 | KLF5 | 1.27 | CD.Fibro.CHI3L1.CXCL3 | DCN | 2.92 | CD.Paneth.DEFA6.ITLN2 | SPINK1 | 0.99 |
| CD.EC.ADH1C.EDN1 | UGT2B17 | 1.24 | CD.Fibro.CHI3L1.CXCL3 | CXCL1 | 3.67 | CD.Paneth.DEFA6.ITLN2 | KLK12 | 0.91 |
| CD.EC.ADH1C.EDN1 | ALDOB | 1.19 | CD.Fibro.COL6A5.CXCL6 | LUM | 4.10 | CD.Paneth.DEFA6.ITLN2 | RETNLB | 0.88 |
| CD.EC.ADH1C.EDN1 | CLDN7 | 1.15 | CD.Fibro.COL6A5.CXCL6 | PTGDS | 3.53 | CD.Paneth.DEFA6.ITLN2 | CH17-125A10.2 | 0.87 |
| CD.EC.ADH1C.EDN1 | AGR3 | 1.13 | CD.Fibro.COL6A5.CXCL6 | DCN | 3.45 | CD.Paneth.DEFA6.ITLN2 | CDKN1C | 0.85 |
| CD.EC.ADH1C.EDN1 | SMIM22 | 1.12 | CD.Fibro.COL6A5.CXCL6 | CCL11 | 3.38 | CD.Paneth.DEFA6.ITLN2 | PCSK1 | 0.83 |
| CD.EC.ADH1C.EDN1 | MGST1 | 1.12 | CD.Fibro.COL6A5.CXCL6 | CCL13 | 3.35 | CD.Paneth.DEFA6.ITLN2 | AZGP1 | 0.81 |
| CD.EC.ADH1C.EDN1 | ELF3 | 1.12 | CD.Fibro.COL6A5.CXCL6 | CFD | 3.30 | CD.Paneth.DEFA6.ITLN2 | TM4SF5 | 0.64 |
| CD.EC.ADH1C.EDN1 | PPP1R1B | 1.07 | CD.Fibro.COL6A5.CXCL6 | CXCL6 | 3.22 | CD.Paneth.DEFA6.ITLN2 | PDIA2 | 0.62 |
| CD.EC.ADH1C.EDN1 | RAMP1 | 1.00 | CD.Fibro.COL6A5.CXCL6 | CXCL1 | 3.14 | CD.Paneth.DEFA6.ITLN2 | PLCG2 | 1.39 |
| CD.EC.ADH1C.EDN1 | FAM3B | 0.99 | CD.Fibro.COL6A5.CXCL6 | MFAP4 | 3.07 | CD.Paneth.DEFA6.ITLN2 | ELF3 | 0.97 |
| CD.EC.ADH1C.EDN1 | GPR160 | 0.99 | CD.Fibro.COL6A5.CXCL6 | RARRES2 | 2.99 | CD.Paneth.DEFA6.ITLN2 | PRUNE2 | 0.54 |
| CD.EC.ADH1C.EDN1 | AKR1C3 | 0.98 | CD.Fibro.COL6A5.CXCL6 | CCL8 | 2.98 | CD.pDC.IRF7.IL3RA | GZMB | 3.13 |
| CD.EC.ADH1C.EDN1 | CES2 | 0.97 | CD.Fibro.COL6A5.CXCL6 | ADH1B | 2.92 | CD.pDC.IRF7.IL3RA | IRF7 | 2.32 |
| CD.EC.ADH1C.EDN1 | MT1M | 0.97 | CD.Fibro.COL6A5.CXCL6 | CXCL14 | 2.83 | CD.pDC.IRF7.IL3RA | LILRA4 | 2.09 |
| CD.EC.ADH1C.EDN1 | DSP | 0.94 | CD.Fibro.COL6A5.CXCL6 | CFH | 2.78 | CD.pDC.IRF7.IL3RA | PLD4 | 2.03 |
| CD.EC.ADH1C.EDN1 | CDX1 | 0.91 | CD.Fibro.COL6A5.CXCL6 | CCL2 | 2.67 | CD.pDC.IRF7.IL3RA | C12orf75 | 1.95 |
| CD.EC.ADH1C.EDN1 | SFN | 0.86 | CD.Fibro.COL6A5.CXCL6 | C1S | 2.65 | CD.pDC.IRF7.IL3RA | CLIC3 | 1.86 |
| CD.EC.ADH1C.EDN1 | MAOA | 0.85 | CD.Fibro.COL6A5.CXCL6 | CALD1 | 2.39 | CD.pDC.IRF7.IL3RA | UGCG | 1.80 |
| CD.EC.ADH1C.EDN1 | PRSS3 | 0.83 | CD.Fibro.COL6A5.CXCL6 | COL1A2 | 2.39 | CD.pDC.IRF7.IL3RA | IL3RA | 1.60 |
| CD.EC.ADH1C.EDN1 | SELENBP1 | 0.83 | CD.Fibro.COL6A5.CXCL6 | PLPP1 | 2.39 | CD.pDC.IRF7.IL3RA | IRF4 | 1.54 |
| CD.EC.ADH1C.EDN1 | RAB25 | 0.81 | CD.Fibro.COL6A5.CXCL6 | FBLN1 | 2.35 | CD.pDC.IRF7.IL3RA | SERPINF1 | 1.49 |
| CD.EC.ADH1C.EDN1 | CDX2 | 0.80 | CD.Fibro.COL6A5.CXCL6 | EDIL3 | 2.34 | CD.pDC.IRF7.IL3RA | SCT | 1.46 |
| CD.EC.ADH1C.EDN1 | CDC42EP5 | 0.80 | CD.Fibro.COL6A5.CXCL6 | COL3A1 | 2.31 | CD.pDC.IRF7.IL3RA | PTPRS | 1.45 |
| CD.EC.ADH1C.EDN1 | GDF15 | 0.79 | CD.Fibro.COL6A5.CXCL6 | C1R | 2.28 | CD.pDC.IRF7.IL3RA | PTCRA | 1.41 |
| CD.EC.ADH1C.EDN1 | TCEA3 | 0.75 | CD.Fibro.COL6A5.CXCL6 | RBP1 | 2.22 | CD.pDC.IRF7.IL3RA | SMPD3 | 1.36 |
| CD.EC.ADH1C.EDN1 | KRTCAP3 | 0.75 | CD.Fibro.COL6A5.CXCL6 | TFPI | 2.20 | CD.pDC.IRF7.IL3RA | MYBL2 | 1.35 |
| CD.EC.ADH1C.EDN1 | CHP2 | 0.75 | CD.Fibro.COL6A5.CXCL6 | CP | 2.07 | CD.pDC.IRF7.IL3RA | LILRB4 | 1.22 |
| CD.EC.ADH1C.EDN1 | IGFBP2 | 0.75 | CD.Fibro.COL6A5.CXCL6 | CXCL12 | 2.05 | CD.pDC.IRF7.IL3RA | MAP1A | 1.04 |
| CD.EC.ADH1C.EDN1 | CMBL | 0.74 | CD.Fibro.COL6A5.CXCL6 | C7 | 2.01 | CD.pDC.IRF7.IL3RA | LRRC26 | 1.04 |
| CD.EC.ADH1C.EDN1 | CLDN15 | 0.73 | CD.Fibro.COL6A5.CXCL6 | COL18A1 | 1.98 | CD.pDC.IRF7.IL3RA | CLEC4C | 1.01 |
| CD.EC.ADH1C.EDN1 | CAMK2N1 | 0.73 | CD.Fibro.COL6A5.CXCL6 | VCAM1 | 1.98 | CD.pDC.IRF7.IL3RA | SUSD1 | 0.96 |
| CD.EC.ADH1C.EDN1 | ST14 | 0.73 | CD.Fibro.COL6A5.CXCL6 | HAPLN1 | 1.97 | CD.pDC.IRF7.IL3RA | LAMP5 | 0.83 |
| CD.EC.ADH1C.EDN1 | DHRS11 | 0.73 | CD.Fibro.COL6A5.CXCL6 | QSOX1 | 1.96 | CD.pDC.IRF7.IL3RA | KRT5 | 0.65 |
| CD.EC.ADH1C.EDN1 | MISP | 0.70 | CD.Fibro.COL6A5.CXCL6 | GPX3 | 1.93 | CD.pDC.IRF7.IL3RA | RP11- | 0.57 |
| | | | | | | | 73G16.2 | |
| CD.EC.ADH1C.EDN1 | PRAP1 | 0.70 | CD.Fibro.COL6A5.CXCL6 | SPARCL1 | 1.92 | CD.pDC.IRF7.IL3RA | EPHB1 | 0.51 |
| CD.EC.ADH1C.EDN1 | SLC39A5 | 0.69 | CD.Fibro.COL6A5.CXCL6 | ANGPTL1 | 1.88 | CD.pDC.IRF7.IL3RA | PACSIN1 | 0.50 |
| CD.EC.ADH1C.EDN1 | PTGR1 | 0.69 | CD.Fibro.COL6A5.CXCL6 | VCAN | 1.87 | CD.pDC.IRF7.IL3RA | RNASE6 | 1.53 |
| CD.EC.ADH1C.EDN1 | HMGCS2 | 0.67 | CD.Fibro.COL6A5.CXCL6 | IGFBP5 | 1.85 | CD.pDC.IRF7.IL3RA | TCF4 | 1.85 |
| CD.EC.ADH1C.EDN1 | URAD | 0.65 | CD.Fibro.COL6A5.CXCL6 | SERPINF1 | 1.83 | CD.pDC.IRF7.IL3RA | PHEX | 0.57 |
| CD.EC.ADH1C.EDN1 | VIL1 | 0.64 | CD.Fibro.COL6A5.CXCL6 | ASPN | 1.82 | CD.pDC.IRF7.IL3RA | PPP1R14B | 1.43 |
| CD.EC.ADH1C.EDN1 | RGMB | 0.64 | CD.Fibro.COL6A5.CXCL6 | PLEKHH2 | 1.82 | CD.pDC.IRF7.IL3RA | PLAC8 | 2.22 |
| CD.EC.ADH1C.EDN1 | SMIM24 | 0.64 | CD.Fibro.COL6A5.CXCL6 | EMILIN1 | 1.81 | CD.pDC.IRF7.IL3RA | TNFRSF21 | 0.77 |
| CD.EC.ADH1C.EDN1 | C2orf82 | 0.63 | CD.Fibro.COL6A5.CXCL6 | PLTP | 1.79 | CD.pDC.IRF7.IL3RA | TSPAN13 | 1.76 |
| CD.EC.ADH1C.EDN1 | KIAA1324 | 0.63 | CD.Fibro.COL6A5.CXCL6 | MMP2 | 1.78 | CD.Plsma/IgA.AL9287683.RPL39 | IGKC | 2.32 |
| CD.EC.ADH1C.EDN1 | TMEM54 | 0.63 | CD.Fibro.COL6A5.CXCL6 | DDR2 | 1.76 | CD.Plsma/IgA.AL9287683.RPL39 | IGHA1 | 2.25 |
| CD.EC.ADH1C.EDN1 | MTRNR2L1 | 0.62 | CD.Fibro.COL6A5.CXCL6 | EDNRB | 1.75 | CD.Plsma/IgA.AL9287683.RPL39 | JCHAIN | 2.11 |
| CD.EC.ADH1C.EDN1 | PDZK1IP1 | 0.62 | CD.Fibro.COL6A5.CXCL6 | PLPP3 | 1.72 | CD.Plsma/IgA.AL9287683.RPL39 | MZB1 | 1.76 |
| CD.EC.ADH1C.EDN1 | DSG2 | 0.62 | CD.Fibro.COL6A5.CXCL6 | CRYBG3 | 1.70 | CD.Plsma/IgA.AL9287683.RPL39 | AL928768.3 | 1.60 |
| CD.EC.ADH1C.EDN1 | GOLM1 | 0.61 | CD.Fibro.COL6A5.CXCL6 | A2M | 1.70 | CD.Plsma/IgA.AL9287683.RPL39 | IGHA2 | 1.45 |
| CD.EC.ADH1C.EDN1 | FAM3D | 0.60 | CD.Fibro.COL6A5.CXCL6 | GGT5 | 1.67 | CD.Plsma/IgA.AL9287683.RPL39 | DERL3 | 1.33 |
| CD.EC.ADH1C.EDN1 | EPHB2 | 0.59 | CD.Fibro.COL6A5.CXCL6 | FBLN5 | 1.64 | CD.Plsma/IgA.AL9287683.RPL39 | SSR4 | 1.33 |
| CD.EC.ADH1C.EDN1 | GPRC5A | 0.59 | CD.Fibro.COL6A5.CXCL6 | CTSK | 1.57 | CD.Plsma/IgA.AL9287683.RPL39 | SEC11C | 1.16 |
| CD.EC.ADH1C.EDN1 | KCNJ13 | 0.58 | CD.Fibro.COL6A5.CXCL6 | RBP5 | 1.57 | CD.Plsma/IgA.AL9287683.RPL39 | FKBP11 | 1.12 |
| CD.EC.ADH1C.EDN1 | LCN2 | 0.58 | CD.Fibro.COL6A5.CXCL6 | NUPR1 | 1.56 | CD.Plsma/IgA.AL9287683.RPL39 | TNFRSF17 | 1.12 |
| CD.EC.ADH1C.EDN1 | TMC5 | 0.57 | CD.Fibro.COL6A5.CXCL6 | FHL2 | 1.56 | CD.Plsma/IgA.AL9287683.RPL39 | DUSP5 | 1.00 |
| CD.EC.ADH1C.EDN1 | ERBB3 | 0.57 | CD.Fibro.COL6A5.CXCL6 | COL6A2 | 1.55 | CD.Plsma/IgA.AL9287683.RPL39 | UBE2J1 | 0.88 |
| CD.EC.ADH1C.EDN1 | UGT2A3 | 0.56 | CD.Fibro.COL6A5.CXCL6 | CYGB | 1.54 | CD.Plsma/IgA.AL9287683.RPL39 | XBP1 | 0.87 |
| CD.EC.ADH1C.EDN1 | CDH17 | 0.55 | CD.Fibro.COL6A5.CXCL6 | CHL1 | 1.54 | CD.Plsma/IgA.AL9287683.RPL39 | HERPUD1 | 0.85 |
| CD.EC.ADH1C.EDN1 | CPS1 | 0.55 | CD.Fibro.COL6A5.CXCL6 | COL6A5 | 1.49 | CD.Plsma/IgA.AL9287683.RPL39 | PRDX4 | 0.79 |
| CD.EC.ADH1C.EDN1 | USH1C | 0.54 | CD.Fibro.COL6A5.CXCL6 | NID1 | 1.48 | CD.Plsma/IgA.AL9287683.RPL39 | ITM2C | 0.76 |
| CD.EC.ADH1C.EDN1 | HNF4A | 0.53 | CD.Fibro.COL6A5.CXCL6 | FSTL1 | 1.46 | CD.Plsma/IgA.AL9287683.RPL39 | MANF | 0.76 |
| CD.EC.ADH1C.EDN1 | PKP2 | 0.53 | CD.Fibro.COL6A5.CXCL6 | PDGFRA | 1.46 | CD.Plsma/IgA.AL9287683.RPL39 | FKBP2 | 0.73 |
| CD.EC.ADH1C.EDN1 | PLPP2 | 0.52 | CD.Fibro.COL6A5.CXCL6 | BMP5 | 1.45 | CD.Plsma/IgA.AL9287683.RPL39 | MYDGF | 0.73 |
| CD.EC.ADH1C.EDN1 | SNHG18 | 0.52 | CD.Fibro.COL6A5.CXCL6 | MEG3 | 1.45 | CD.Plsma/IgA.AL9287683.RPL39 | VIMP | 0.72 |
| CD.EC.ADH1C.EDN1 | ATP8B1 | 0.51 | CD.Fibro.COL6A5.CXCL6 | PCOLCE | 1.42 | CD.Plsma/IgA.AL9287683.RPL39 | SSR3 | 0.71 |
| CD.EC.ADH1C.EDN1 | PRSS8 | 0.51 | CD.Fibro.COL6A5.CXCL6 | TDO2 | 1.41 | CD.Plsma/IgA.AL9287683.RPL39 | SDF2L1 | 0.70 |
| CD.EC.ADH1C.EDN1 | DDC | 0.50 | CD.Fibro.COL6A5.CXCL6 | PROS1 | 1.40 | CD.Plsma/IgA.AL9287683.RPL39 | PDIA4 | 0.69 |
| CD.EC.ADH1C.EDN1 | TMEM45B | 0.49 | CD.Fibro.COL6A5.CXCL6 | ALDH1A3 | 1.39 | CD.Plsma/IgA.AL9287683.RPL39 | CRELD2 | 0.68 |
| CD.EC.ADH1C.EDN1 | C10orf99 | 0.49 | CD.Fibro.COL6A5.CXCL6 | ABI3BP | 1.39 | CD.Plsma/IgA.AL9287683.RPL39 | DNAJB9 | 0.62 |
| CD.EC.ADH1C.EDN1 | STAP2 | 0.48 | CD.Fibro.COL6A5.CXCL6 | SGCE | 1.38 | CD.Plsma/IgA.AL9287683.RPL39 | SPCS3 | 0.56 |
| CD.EC.ADH1C.EDN1 | IMPA2 | 0.48 | CD.Fibro.COL6A5.CXCL6 | SFRP1 | 1.37 | CD.Plsma/IgA.AL9287683.RPL39 | CCR10 | 0.51 |
| CD.EC.ADH1C.EDN1 | AQP1 | 0.47 | CD.Fibro.COL6A5.CXCL6 | FIBIN | 1.33 | CD.Plsma/IgA.AL9287683.RPL39 | EAF2 | 0.47 |
| CD.EC.ADH1C.EDN1 | SI | 0.47 | CD.Fibro.COL6A5.CXCL6 | EFEMP1 | 1.32 | CD.Plsma/IgA.AL9287683.RPL39 | PLPP5 | 0.44 |
| CD.EC.ADH1C.EDN1 | TST | 0.47 | CD.Fibro.COL6A5.CXCL6 | FAM198B | 1.30 | CD.Plsma/IgA.AL9287683.RPL39 | TXNDC11 | 0.43 |
| CD.EC.ADH1C.EDN1 | STXBP6 | 0.46 | CD.Fibro.COL6A5.CXCL6 | PCDH7 | 1.24 | CD.Plsma/IgA.AL9287683.RPL39 | CHPF | 0.36 |
| CD.EC.ADH1C.EDN1 | GGT6 | 0.46 | CD.Fibro.COL6A5.CXCL6 | SFTA1P | 1.23 | CD.Plsma/IgA.AL9287683.RPL39 | MEI1 | 0.35 |
| CD.EC.ADH1C.EDN1 | FERMT1 | 0.46 | CD.Fibro.COL6A5.CXCL6 | TCF21 | 1.22 | CD.Plsma/IgA.AL9287683.RPL39 | GNG7 | 0.34 |
| CD.EC.ADH1C.EDN1 | GJB1 | 0.45 | CD.Fibro.COL6A5.CXCL6 | COL6A3 | 1.22 | CD.Plsma/IgA.AL9287683.RPL39 | TNFRSF13B | 0.29 |
| CD.EC.ADH1C.EDN1 | LAD1 | 0.45 | CD.Fibro.COL6A5.CXCL6 | LIFR | 1.22 | CD.Plsma/IgA.AL9287683.RPL39 | ERLEC1 | 0.41 |
| CD.EC.ADH1C.EDN1 | CFTR | 0.45 | CD.Fibro.COL6A5.CXCL6 | SNAI2 | 1.21 | CD.Plsma/IgA.AL9287683.RPL39 | CD79A | 0.36 |
| CD.EC.ADH1C.EDN1 | OSR2 | 0.45 | CD.Fibro.COL6A5.CXCL6 | PCDH18 | 1.20 | CD.Plsma/IgA.AL9287683.RPL39 | UAP1 | 0.35 |
| CD.EC.ADH1C.EDN1 | SOX9 | 0.45 | CD.Fibro.COL6A5.CXCL6 | FBN1 | 1.12 | CD.Plsma/IgA.AL9287683.RPL39 | SRM | 0.36 |
| CD.EC.ADH1C.EDN1 | GIPC2 | 0.45 | CD.Fibro.COL6A5.CXCL6 | PTN | 1.12 | CD.Plsma/IgA.AL9287683.RPL39 | PABPC4 | 0.40 |
| CD.EC.ADH1C.EDN1 | AP1M2 | 0.44 | CD.Fibro.COL6A5.CXCL6 | ABCA8 | 1.10 | CD.Plsma/IgA.AL9287683.RPL39 | HSP90B1 | 0.86 |
| CD.EC.ADH1C.EDN1 | DEGS2 | 0.44 | CD.Fibro.COL6A5.CXCL6 | CH25H | 1.08 | CD.Plsma/IgA.AL9287683.RPL39 | HM13 | 0.38 |
| CD.EC.ADH1C.EDN1 | CCL15 | 0.44 | CD.Fibro.COL6A5.CXCL6 | ADGRL2 | 1.07 | CD.Plsma/IgA.AL9287683.RPL39 | TXNDC15 | 0.36 |
| CD.EC.ADH1C.EDN1 | EPHA2 | 0.44 | CD.Fibro.COL6A5.CXCL6 | CDH11 | 1.06 | CD.Plsma/IgA.AL9287683.RPL39 | SPATS2 | 0.31 |
| CD.EC.ADH1C.EDN1 | VDR | 0.43 | CD.Fibro.COL6A5.CXCL6 | FNDC1 | 0.95 | CD.Plsma/IgA.AL9287683.RPL39 | FAM46C | 0.31 |
| CD.EC.ADH1C.EDN1 | PTPRF | 0.43 | CD.Fibro.COL6A5.CXCL6 | NEGR1 | 0.93 | CD.Plsma/IgA.HSPA1B.HSPA1A | IGHA1 | 2.48 |
| CD.EC.ADH1C.EDN1 | AKR7A3 | 0.42 | CD.Fibro.COL6A5.CXCL6 | BCHE | 0.87 | CD.Plsma/IgA.HSPA1B.HSPA1A | IGKC | 2.21 |
| CD.EC.ADH1C.EDN1 | TMPRSS2 | 0.41 | CD.Fibro.COL6A5.CXCL6 | PAPPA | 0.85 | CD.Plsma/IgA.HSPA1B.HSPA1A | IGHA2 | 2.19 |
| CD.EC.ADH1C.EDN1 | VSIG10 | 0.40 | CD.Fibro.COL6A5.CXCL6 | TAC3 | 0.81 | CD.Plsma/IgA.HSPA1B.HSPA1A | JCHAIN | 2.18 |
| CD.EC.ADH1C.EDN1 | ABO | 0.39 | CD.Fibro.COL6A5.CXCL6 | EPHA7 | 0.75 | CD.Plsma/IgA.HSPA1B.HSPA1A | JUN | 1.56 |
| CD.EC.ADH1C.EDN1 | PLCB4 | 0.39 | CD.Fibro.COL6A5.CXCL6 | NNMT | 1.40 | CD.Plsma/IgA.HSPA1B.HSPA1A | MZB1 | 1.42 |
| CD.EC.ADH1C.EDN1 | PPP1R14D | 0.39 | CD.Fibro.COL6A5.CXCL6 | SPARC | 1.48 | CD.Plsma/IgA.HSPA1B.HSPA1A | HSPA1A | 1.39 |
| CD.EC.ADH1C.EDN1 | OVOL1 | 0.38 | CD.Fibro.COL6A5.CXCL6 | ADAMDEC1 | 3.88 | CD.Plsma/IgA.HSPA1B.HSPA1A | AL928768.3 | 1.35 |
| CD.EC.ADH1C.EDN1 | MUC3A | 0.37 | CD.Fibro.COL6A5.CXCL6 | DPT | 0.93 | CD.Plsma/IgA.HSPA1B.HSPA1A | SSR4 | 1.28 |
| CD.EC.ADH1C.EDN1 | FABP2 | 0.36 | CD.Fibro.COL6A5.CXCL6 | FXYD1 | 1.25 | CD.Plsma/IgA.HSPA1B.HSPA1A | HSPA1B | 1.26 |
| CD.EC.ADH1C.EDN1 | POF1B | 0.36 | CD.Fibro.COL6A5.CXCL6 | SHISA3 | 0.70 | CD.Plsma/IgA.HSPA1B.HSPA1A | FKBP11 | 1.15 |
| CD.EC.ADH1C.EDN1 | CHDH | 0.34 | CD.Fibro.COL6A5.CXCL6 | ABCA6 | 1.04 | CD.Plsma/IgA.HSPA1B.HSPA1A | DERL3 | 1.12 |
| CD.EC.ADH1C.EDN1 | ERBB2 | 0.34 | CD.Fibro.COL6A5.CXCL6 | KITLG | 1.03 | CD.Plsma/IgA.HSPA1B.HSPA1A | HERPUD1 | 1.05 |
| CD.EC.ADH1C.EDN1 | TMEM171 | 0.34 | CD.Fibro.COL6A5.CXCL6 | FOXF1 | 0.88 | CD.Plsma/IgA.HSPA1B.HSPA1A | TNFRSF17 | 1.05 |
| CD.EC.ADH1C.EDN1 | F2RL1 | 0.32 | CD.Fibro.COL6A5.CXCL6 | TPM2 | 1.32 | CD.Plsma/IgA.HSPA1B.HSPA1A | SEC11C | 1.03 |
| CD.EC.ADH1C.EDN1 | ARSE | 0.32 | CD.Fibro.COL6A5.CXCL6 | SMPDL3A | 1.69 | CD.Plsma/IgA.HSPA1B.HSPA1A | DUSP5 | 1.02 |
| CD.EC.ADH1C.EDN1 | PROM1 | 0.29 | CD.Fibro.COL6A5.CXCL6 | CD302 | 1.69 | CD.Plsma/IgA.HSPA1B.HSPA1A | XBP1 | 0.94 |
| CD.EC.ADH1C.EDN1 | SLC13A2 | 0.25 | CD.Fibro.COL6A5.CXCL6 | FHL1 | 1.42 | CD.Plsma/IgA.HSPA1B.HSPA1A | ANKRD28 | 0.85 |
| CD.EC.ADH1C.EDN1 | FUT3 | 0.34 | CD.Fibro.COL6A5.CXCL6 | COL14A1 | 1.16 | CD.Plsma/IgA.HSPA1B.HSPA1A | PRDX4 | 0.81 |
| CD.EC.ADH1C.EDN1 | PKP3 | 0.36 | CD.Fibro.COL6A5.CXCL6 | COL1A1 | 1.20 | CD.Plsma/IgA.HSPA1B.HSPA1A | ITM2C | 0.78 |
| CD.EC.ADH1C.EDN1 | ID1 | 1.20 | CD.Fibro.COL6A5.CXCL6 | SULF1 | 0.96 | CD.Plsma/IgA.HSPA1B.HSPA1A | UBE2J1 | 0.74 |
| CD.EC.ADH1C.EDN1 | TRIM2 | 0.41 | CD.Fibro.COL6A5.CXCL6 | IGF1 | 1.71 | CD.Plsma/IgA.HSPA1B.HSPA1A | DNAJB9 | 0.72 |
| CD.EC.ADH1C.EDN1 | SLC44A4 | 0.52 | CD.Fibro.COL6A5.CXCL6 | CYBRD1 | 1.20 | CD.Plsma/IgA.HSPA1B.HSPA1A | PIM2 | 0.70 |
| CD.EC.ADH1C.EDN1 | PPP1R16A | 0.58 | CD.Fibro.COL6A5.SLITRK6 | COL6A5 | 2.07 | CD.Plsma/IgA.HSPA1B.HSPA1A | ANKRD37 | 0.70 |
| CD.EC.ADH1C.EDN1 | SH2D4A | 0.33 | CD.Fibro.COL6A5.SLITRK6 | SLITRK6 | 1.50 | CD.Plsma/IgA.HSPA1B.HSPA1A | SSR3 | 0.63 |
| CD.EC.ADH1C.EDN1 | CERS6 | 0.63 | CD.Fibro.CPM.HSD17B2 | APOD | 2.40 | CD.Plsma/IgA.HSPA1B.HSPA1A | MANF | 0.59 |
| CD.EC.ADH1C.EDN1 | AGR2 | 1.35 | CD.Fibro.CPM.HSD17B2 | PTGIS | 1.03 | CD.Plsma/IgA.HSPA1B.HSPA1A | SDC1 | 0.56 |
| CD.EC.ADH1C.EDN1 | C6orf132 | 0.31 | CD.Fibro.CPM.HSD17B2 | FIGF | 0.97 | CD.Plsma/IgA.HSPA1B.HSPA1A | TRIB1 | 0.56 |
| CD.EC.ADH1C.EDN1 | CYP3A5 | 0.61 | CD.Fibro.CXCL14.DEFA5 | C1S | 2.59 | CD.Plsma/IgA.HSPA1B.HSPA1A | EGR1 | 0.52 |
| CD.EC.ADH1C.EDN1 | TMC4 | 0.33 | CD.Fibro.CXCL14.DEFA5 | LUM | 2.52 | CD.Plsma/IgA.HSPA1B.HSPA1A | CRELD2 | 0.51 |
| CD.EC.ADH1C.EDN1 | RP11- | 0.52 | CD.Fibro.CXCL14.DEFA5 | DCN | 2.01 | CD.Plsma/IgA.HSPA1B.HSPA1A | PABPC4 | 0.51 |
| | 357H14.17 | | | | | | | |
| CD.EC.ADH1C.EDN1 | TRABD2A | 0.45 | CD.Fibro.CXCL14.DEFA5 | WFDC1 | 1.53 | CD.Plsma/IgA.HSPA1B.HSPA1A | FAM46C | 0.46 |
| CD.EC.ADH1C.EDN1 | HSD11B2 | 0.39 | CD.Fibro.CXCL14.DEFA5 | CXCL14 | 3.95 | CD.Plsma/IgA.HSPA1B.HSPA1A | EAF2 | 0.46 |
| CD.EC.ADH1C.EDN1 | EPHX2 | 0.42 | CD.Fibro.CXCL14.DEFA5 | MFAP4 | 1.80 | CD.Plsma/IgA.HSPA1B.HSPA1A | CCR10 | 0.43 |
| CD.EC.ADH1C.EDN1 | LGR5 | 0.28 | CD.Fibro.CXCL14.DEFA5 | CALD1 | 2.46 | CD.Plsma/IgA.HSPA1B.HSPA1A | CHPF | 0.42 |
| CD.EC.ADH1C.EDN1 | REG1B | 1.66 | CD.Fibro.CXCL14.DEFA5 | TCF21 | 1.80 | CD.Plsma/IgA.HSPA1B.HSPA1A | SLAMF7 | 0.41 |
| CD.EC.ADH1C.EDN1 | FAM84A | 0.43 | CD.Fibro.CXCL14.DEFA5 | MMP2 | 1.82 | CD.Plsma/IgA.HSPA1B.HSPA1A | FCRL5 | 0.39 |
| CD.EC.ADH1C.EDN1 | STARD10 | 0.61 | CD.Fibro.CXCL14.DEFA5 | C1R | 2.35 | CD.Plsma/IgA.HSPA1B.HSPA1A | TXNDC11 | 0.39 |
| CD.EC.ADH1C.EDN1 | TMPRSS4 | 0.34 | CD.Fibro.CXCL14.DEFA5 | RARRES2 | 2.73 | CD.Plsma/IgA.HSPA1B.HSPA1A | GNG7 | 0.38 |
| CD.EC.ADH1C.EDN1 | CLDN2 | 0.31 | CD.Fibro.DEFA6.DEFA5 | LUM | 2.65 | CD.Plsma/IgA.HSPA1B.HSPA1A | PLPP5 | 0.37 |
| CD.EC.ADH1C.EDN1 | PLCB3 | 0.46 | CD.Fibro.F3.SOX6 | PDGFRA | 3.01 | CD.Plsma/IgA.HSPA1B.HSPA1A | TXNDC15 | 0.37 |
| CD.EC.ADH1C.EDN1 | SH3D19 | 0.37 | CD.Fibro.FIGF.MMP11 | CXCL14 | 3.83 | CD.Plsma/IgA.HSPA1B.HSPA1A | RP11- | 0.35 |
| | | | | | | | 290F5.1 | |
| CD.EC.ADH1C.EDN1 | ANPEP | 0.70 | CD.Fibro.FIGF.MMP11 | COL6A2 | 3.17 | CD.Plsma/IgA.HSPA1B.HSPA1A | POU2AF1 | 0.33 |
| CD.EC.ADH1C.EDN1 | TMEM106C | 0.76 | CD.Fibro.FIGF.MMP11 | CALD1 | 2.84 | CD.Plsma/IgA.HSPA1B.HSPA1A | MEI1 | 0.33 |
| CD.EC.ADH1C.EDN1 | REEP6 | 0.47 | CD.Fibro.FIGF.MMP11 | EDNRB | 2.81 | CD.Plsma/IgA.HSPA1B.HSPA1A | SPAG4 | 0.31 |
| CD.EC.ADH1C.EDN1 | GATA6 | 0.31 | CD.Fibro.FIGF.MMP11 | NBL1 | 2.78 | CD.Plsma/IgA.HSPA1B.HSPA1A | PDK1 | 0.26 |
| CD.EC.ADH1C.EDN1 | AKAP1 | 0.45 | CD.Fibro.FIGF.MMP11 | TAGLN | 2.77 | CD.Plsma/IgA.HSPA1B.HSPA1A | WNT10A | 0.26 |
| CD.EC.ADH1C.GSTA1 | ADH1C | 1.72 | CD.Fibro.FIGF.MMP11 | COL1A1 | 2.76 | CD.Plsma/IgA.HSPA1B.HSPA1A | IGKV1-12 | 0.36 |
| CD.EC.ADH1C.GSTA1 | KCNJ13 | 0.88 | CD.Fibro.FIGF.MMP11 | COL6A1 | 2.74 | CD.Plsma/IgA.HSPA1B.HSPA1A | PAIP2B | 0.28 |
| CD.EC.ADH1C.GSTA1 | SERPINB5 | 0.79 | CD.Fibro.FIGF.MMP11 | COL1A2 | 2.72 | CD.Plsma/IgA.HSPA1B.HSPA1A | PDIA4 | 0.52 |
| CD.EC.ADH1C.GSTA1 | AKR1B10 | 0.65 | CD.Fibro.FIGF.MMP11 | APOD | 2.63 | CD.Plsma/IgA.HSPA1B.HSPA1A | HDLBP | 0.31 |
| CD.EC.ADH1C.GSTA1 | CKMT1A | 0.56 | CD.Fibro.FIGF.MMP11 | COL3A1 | 2.63 | CD.Plsma/IgA.HSPA1B.HSPA1A | SDF2L1 | 0.52 |
| CD.EC.ADH1C.GSTA1 | GSTA1 | 1.84 | CD.Fibro.FIGF.MMP11 | IGFBP7 | 2.63 | CD.Plsma/IgA.HSPA1B.HSPA1A | GLA | 0.60 |
| CD.EC.ADH1C.RPS4Y1 | AKR1C3 | 1.77 | CD.Fibro.FIGF.MMP11 | FRZB | 2.47 | CD.Plsma/IgA.HSPA1B.HSPA1A | UAP1 | 0.28 |
| CD.EC.ADH1C.RPS4Y1 | GSTA1 | 1.60 | CD.Fibro.FIGF.MMP11 | BMP4 | 2.39 | CD.Plsma/IgA.HSPA1B.HSPA1A | VIMP | 0.57 |
| CD.EC.ADH1C.RPS4Y1 | ADH1C | 1.59 | CD.Fibro.FIGF.MMP11 | MMP2 | 2.36 | CD.Plsma/IgA.HSPA1B.HSPA1A | HSP90B1 | 0.85 |
| CD.EC.ADH1C.RPS4Y1 | MAOA | 1.04 | CD.Fibro.FIGF.MMP11 | PDGFRA | 2.36 | CD.Plsma/IgA.IGHA1.IGKC | IGHA1 | 2.69 |
| CD.EC.ADH1C.RPS4Y1 | PPP1R1B | 1.02 | CD.Fibro.FIGF.MMP11 | MFAP4 | 2.34 | CD.Plsma/IgA.IGHA1.IGKC | IGKC | 2.59 |
| CD.EC.ADH1C.RPS4Y1 | DHRS11 | 1.00 | CD.Fibro.FIGF.MMP11 | SPARC | 2.33 | CD.Plsma/IgA.IGHA1.IGKC | JCHAIN | 2.27 |
| CD.EC.ADH1C.RPS4Y1 | CPS1 | 0.93 | CD.Fibro.FIGF.MMP11 | F3 | 2.31 | CD.Plsma/IgA.IGHA1.IGKC | IGHA2 | 2.11 |
| CD.EC.ADH1C.RPS4Y1 | CHP2 | 0.87 | CD.Fibro.FIGF.MMP11 | IGFBP5 | 2.28 | CD.Plsma/IgA.IGHA1.IGKC | MZB1 | 1.48 |
| CD.EC.ADH1C.RPS4Y1 | KCNJ13 | 0.84 | CD.Fibro.FIGF.MMP11 | ACTA2 | 2.26 | CD.Plsma/IgA.IGHA1.IGKC | DERL3 | 1.46 |
| CD.EC.ADH1C.RPS4Y1 | DDC | 0.73 | CD.Fibro.FIGF.MMP11 | CPM | 2.25 | CD.Plsma/IgA.IGHA1.IGKC | SSR4 | 1.26 |
| CD.EC.ADH1C.RPS4Y1 | OTC | 0.71 | CD.Fibro.FIGF.MMP11 | POSTN | 2.20 | CD.Plsma/IgA.IGHA1.IGKC | XBP1 | 1.26 |
| CD.EC.ADH1C.RPS4Y1 | AP1M2 | 0.67 | CD.Fibro.FIGF.MMP11 | C15 | 2.19 | CD.Plsma/IgA.IGHA1.IGKC | FKBP11 | 1.15 |
| CD.EC.ADH1C.RPS4Y1 | CKMT1A | 0.57 | CD.Fibro.FIGF.MMP11 | SDC2 | 2.15 | CD.Plsma/IgA.IGHA1.IGKC | TNFRSF17 | 1.05 |
| CD.EC.ADH1C.RPS4Y1 | TMEM45B | 0.72 | CD.Fibro.FIGF.MMP11 | MYL9 | 2.13 | CD.Plsma/IgA.IGHA1.IGKC | HERPUD1 | 0.90 |
| CD.EC.ADH1C.RPS4Y1 | RAB25 | 0.77 | CD.Fibro.FIGF.MMP11 | OLFML3 | 2.10 | CD.Plsma/IgA.IGHA1.IGKC | PRDX4 | 0.78 |
| CD.EC.ADH1C.RPS4Y1 | PTGR1 | 1.01 | CD.Fibro.FIGF.MMP11 | MMP11 | 2.09 | CD.Plsma/IgA.IGHA1.IGKC | SEC11C | 0.74 |
| CD.EC.ADH1C.RPS4Y1 | CDX1 | 1.03 | CD.Fibro.FIGF.MMP11 | C1R | 2.06 | CD.Plsma/IgA.IGHA1.IGKC | JUN | 0.70 |
| CD.EC.ADH1C.RPS4Y1 | MGST1 | 1.90 | CD.Fibro.FIGF.MMP11 | ADH1B | 2.02 | CD.Plsma/IgA.IGHA1.IGKC | PIM2 | 0.68 |
| CD.EC.ADH1C.RPS4Y1 | AKR1B10 | 0.50 | CD.Fibro.FIGF.MMP11 | TPM2 | 2.01 | CD.Plsma/IgA.IGHA1.IGKC | ITM2C | 0.64 |
| CD.EC.ADH1C.RPS4Y1 | KRTCAP3 | 0.96 | CD.Fibro.FIGF.MMP11 | CAV1 | 1.99 | CD.Plsma/IgA.IGHA1.IGKC | AL928768.3 | 0.60 |
| CD.EC.ADH1C.RPS4Y1 | CDH17 | 0.85 | CD.Fibro.FIGF.MMP11 | RARRES2 | 1.96 | CD.Plsma/IgA.IGHA1.IGKC | ANKRD28 | 0.56 |
| CD.EC.AKR1C1.TSPAN1 | AKR1C1 | 0.75 | CD.Fibro.FIGF.MMP11 | SPARCL1 | 1.93 | CD.Plsma/IgA.IGHA1.IGKC | VIMP | 0.53 |
| CD.EC.ANPEP.DUOX2 | PRAP1 | 2.98 | CD.Fibro.FIGF.MMP11 | PDGFD | 1.87 | CD.Plsma/IgA.IGHA1.IGKC | SSR3 | 0.53 |
| CD.EC.ANPEP.DUOX2 | ANPEP | 2.70 | CD.Fibro.FIGF.MMP11 | FIGF | 1.85 | CD.Plsma/IgA.IGHA1.IGKC | DNAJB9 | 0.52 |
| CD.EC.ANPEP.DUOX2 | PHGR1 | 2.64 | CD.Fibro.FIGF.MMP11 | CYGB | 1.84 | CD.Plsma/IgA.IGHA1.IGKC | FKBP2 | 0.52 |
| CD.EC.ANPEP.DUOX2 | FABP1 | 2.43 | CD.Fibro.FIGF.MMP11 | IGFBP6 | 1.83 | CD.Plsma/IgA.IGHA1.IGKC | UBE2J1 | 0.47 |
| CD.EC.ANPEP.DUOX2 | ALDOB | 2.42 | CD.Fibro.FIGF.MMP11 | A2M | 1.80 | CD.Plsma/IgA.IGHA1.IGKC | CRELD2 | 0.46 |
| CD.EC.ANPEP.DUOX2 | CDHR5 | 2.18 | CD.Fibro.FIGF.MMP11 | TMEM119 | 1.78 | CD.Plsma/IgA.IGHA1.IGKC | MANF | 0.45 |
| CD.EC.ANPEP.DUOX2 | KRT8 | 2.09 | CD.Fibro.FIGF.MMP11 | EMILIN1 | 1.76 | CD.Plsma/IgA.IGHA1.IGKC | ERLEC1 | 0.41 |
| CD.EC.ANPEP.DUOX2 | MISP | 1.80 | CD.Fibro.FIGF.MMP11 | GPX3 | 1.74 | CD.Plsma/IgA.IGHA1.IGKC | SPCS3 | 0.40 |
| CD.EC.ANPEP.DUOX2 | KRT20 | 1.69 | CD.Fibro.FIGF.MMP11 | FAM150B | 1.72 | CD.Plsma/IgA.IGHA1.IGKC | TXNDC15 | 0.39 |
| CD.EC.ANPEP.DUOX2 | SI | 1.65 | CD.Fibro.FIGF.MMP11 | FHL1 | 1.72 | CD.Plsma/IgA.IGHA1.IGKC | PLPP5 | 0.36 |
| CD.EC.ANPEP.DUOX2 | SMIM24 | 1.59 | CD.Fibro.FIGF.MMP11 | LAMA4 | 1.71 | CD.Plsma/IgA.IGHA1.IGKC | TXNDC11 | 0.35 |
| CD.EC.ANPEP.DUOX2 | MYO1A | 1.58 | CD.Fibro.FIGF.MMP11 | DMKN | 1.71 | CD.Plsma/IgA.IGHA1.IGKC | SEL1L | 0.33 |
| CD.EC.ANPEP.DUOX2 | MYH14 | 1.45 | CD.Fibro.FIGF.MMP11 | TSHZ2 | 1.69 | CD.Plsma/IgA.IGHA1.IGKC | RP11- | 0.32 |
| | | | | | | | 290F5.1 | |
| CD.EC.ANPEP.DUOX2 | DUOX2 | 1.39 | CD.Fibro.FIGF.MMP11 | BMP5 | 1.69 | CD.Plsma/IgA.IGHA1.IGKC | SDC1 | 0.32 |
| CD.EC.ANPEP.DUOX2 | FABP6 | 2.30 | CD.Fibro.FIGF.MMP11 | PLAT | 1.67 | CD.Plsma/IgA.IGHA1.IGKC | FCRL5 | 0.31 |
| CD.EC.ANPEP.DUOX2 | ADIRF | 2.06 | CD.Fibro.FIGF.MMP11 | SOD3 | 1.65 | CD.Plsma/IgA.IGHA1.IGKC | ALG5 | 0.29 |
| CD.EC.ANPEP.DUOX2 | PIGR | 2.20 | CD.Fibro.FIGF.MMP11 | NPY | 1.61 | CD.Plsma/IgA.IGHA1.IGKC | RP11- | 0.28 |
| | | | | | | | 16E12.2 | |
| CD.EC.ANPEP.DUOX2 | RBP2 | 1.67 | CD.Fibro.FIGF.MMP11 | COX7A1 | 1.61 | CD.Plsma/IgA.IGHA1.IGKC | SPAG4 | 0.27 |
| CD.EC.ANPEP.DUOX2 | ELF3 | 1.88 | CD.Fibro.FIGF.MMP11 | PCOLCE | 1.60 | CD.Plsma/IgA.IGHA1.IGKC | LMAN1 | 0.32 |
| CD.EC.APOB.APOC3 | APOA1 | 4.73 | CD.Fibro.FIGF.MMP11 | COL6A3 | 1.57 | CD.Plsma/IgA.IGHA1.IGKC | SDF2L1 | 0.37 |
| CD.EC.APOB.APOC3 | APOA4 | 4.45 | CD.Fibro.FIGF.MMP11 | MXRA8 | 1.57 | CD.Plsma/IgA.IGHA1.IGKC | SLAMF7 | 0.27 |
| CD.EC.APOB.APOC3 | APOC3 | 3.94 | CD.Fibro.FIGF.MMP11 | DPT | 1.57 | CD.Plsma/IgA.IGHA1.IGKC | SPCS2 | 0.52 |
| CD.EC.APOB.APOC3 | APOB | 3.71 | CD.Fibro.FIGF.MMP11 | SGCE | 1.53 | CD.Plsma/IgA.IGHA1.IGLV6-57 | MZB1 | 1.64 |
| CD.EC.APOB.APOC3 | FABP6 | 3.60 | CD.Fibro.FIGF.MMP11 | ENHO | 1.50 | CD.Plsma/IgA.IGHA1.IGLV6-57 | DERL3 | 1.46 |
| CD.EC.APOB.APOC3 | PRAP1 | 3.36 | CD.Fibro.FIGF.MMP11 | FOXF1 | 1.48 | CD.Plsma/IgA.IGHA1.IGLV6-57 | TNFRSF17 | 1.07 |
| CD.EC.APOB.APOC3 | GUCAZA | 3.25 | CD.Fibro.FIGF.MMP11 | ABI3BP | 1.46 | CD.Plsma/IgA.IGHA1.IGLV6-57 | FKBP11 | 1.29 |
| CD.EC.APOB.APOC3 | FABP2 | 3.16 | CD.Fibro.FIGF.MMP11 | AGT | 1.46 | CD.Plsma/IgA.IGHA1.IGLV6-57 | AL928768.3 | 0.69 |
| CD.EC.APOB.APOC3 | ANPEP | 3.03 | CD.Fibro.FIGF.MMP11 | CFH | 1.46 | CD.Plsma/IgA.IGHA2.IGKV1-12 | IGHA2 | 3.45 |
| CD.EC.APOB.APOC3 | ALDOB | 2.97 | CD.Fibro.FIGF.MMP11 | HSD17B2 | 1.45 | CD.Plsma/IgA.IGHA2.IGKV1-12 | IGHA1 | 2.47 |
| CD.EC.APOB.APOC3 | FABP1 | 2.95 | CD.Fibro.FIGF.MMP11 | NDUFA4L2 | 1.44 | CD.Plsma/IgA.IGHA2.IGKV1-12 | IGKC | 2.39 |
| CD.EC.APOB.APOC3 | CDHR5 | 2.77 | CD.Fibro.FIGF.MMP11 | SOX6 | 1.43 | CD.Plsma/IgA.IGHA2.IGKV1-12 | JCHAIN | 2.12 |
| CD.EC.APOB.APOC3 | SERPINA1 | 2.71 | CD.Fibro.FIGF.MMP11 | LIMCH1 | 1.41 | CD.Plsma/IgA.IGHA2.IGKV1-12 | IGKV1-12 | 1.81 |
| CD.EC.APOB.APOC3 | DPEP1 | 2.65 | CD.Fibro.FIGF.MMP11 | NSG1 | 1.34 | CD.Plsma/IgA.IGHA2.IGKV1-12 | IGKV4-1 | 1.72 |
| CD.EC.APOB.APOC3 | MTTP | 2.59 | CD.Fibro.FIGF.MMP11 | MAMDC2 | 1.34 | CD.Plsma/IgA.IGHA2.IGKV1-12 | IGKV1-5 | 1.44 |
| CD.EC.APOB.APOC3 | ADIRF | 2.49 | CD.Fibro.FIGF.MMP11 | PCSK6 | 1.34 | CD.Plsma/IgA.IGHA2.IGKV1-12 | IGKV3-15 | 1.40 |
| CD.EC.APOB.APOC3 | CUBN | 2.40 | CD.Fibro.FIGF.MMP11 | EFEMP2 | 1.32 | CD.Plsma/IgA.IGHA2.IGKV1-12 | MZB1 | 1.37 |
| CD.EC.APOB.APOC3 | AMN | 2.37 | CD.Fibro.FIGF.MMP11 | BGN | 1.32 | CD.Plsma/IgA.IGHA2.IGKV1-12 | DERL3 | 1.28 |
| CD.EC.APOB.APOC3 | PLS1 | 2.32 | CD.Fibro.FIGF.MMP11 | ECM1 | 1.32 | CD.Plsma/IgA.IGHA2.IGKV1-12 | IGKV3-20 | 1.27 |
| CD.EC.APOB.APOC3 | SLC26A3 | 2.25 | CD.Fibro.FIGF.MMP11 | SMOC2 | 1.32 | CD.Plsma/IgA.IGHA2.IGKV1-12 | SSR4 | 1.26 |
| CD.EC.APOB.APOC3 | DGAT1 | 2.21 | CD.Fibro.FIGF.MMP11 | ACTG2 | 1.30 | CD.Plsma/IgA.IGHA2.IGKV1-12 | TNFRSF17 | 1.19 |
| CD.EC.APOB.APOC3 | AGPAT2 | 2.18 | CD.Fibro.FIGF.MMP11 | TRPA1 | 1.30 | CD.Plsma/IgA.IGHA2.IGKV1-12 | IGKV2-30 | 1.13 |
| CD.EC.APOB.APOC3 | ACE | 2.17 | CD.Fibro.FIGF.MMP11 | TWSG1 | 1.29 | CD.Plsma/IgA.IGHA2.IGKV1-12 | IGKV3-11 | 1.11 |
| CD.EC.APOB.APOC3 | RBP2 | 2.15 | CD.Fibro.FIGF.MMP11 | FN1 | 1.28 | CD.Plsma/IgA.IGHA2.IGKV1-12 | FKBP11 | 1.05 |
| CD.EC.APOB.APOC3 | PCK1 | 2.07 | CD.Fibro.FIGF.MMP11 | COL12A1 | 1.28 | CD.Plsma/IgA.IGHA2.IGKV1-12 | XBP1 | 1.05 |
| CD.EC.APOB.APOC3 | ACSL5 | 2.06 | CD.Fibro.FIGF.MMP11 | SULF1 | 1.26 | CD.Plsma/IgA.IGHA2.IGKV1-12 | PRDX4 | 0.94 |
| CD.EC.APOB.APOC3 | SLC6A19 | 2.03 | CD.Fibro.FIGF.MMP11 | CRISPLD2 | 1.25 | CD.Plsma/IgA.IGHA2.IGKV1-12 | SEC11C | 0.94 |
| CD.EC.APOB.APOC3 | MEP1A | 2.03 | CD.Fibro.FIGF.MMP11 | NDN | 1.24 | CD.Plsma/IgA.IGHA2.IGKV1-12 | ITM2C | 0.74 |
| CD.EC.APOB.APOC3 | MUC13 | 2.03 | CD.Fibro.FIGF.MMP11 | TNC | 1.23 | CD.Plsma/IgA.IGHA2.IGKV1-12 | AL928768.3 | 0.70 |
| CD.EC.APOB.APOC3 | SMIM24 | 2.01 | CD.Fibro.FIGF.MMP11 | TGFB111 | 1.23 | CD.Plsma/IgA.IGHA2.IGKV1-12 | IGLV3-1 | 0.39 |
| CD.EC.APOB.APOC3 | SI | 1.94 | CD.Fibro.FIGF.MMP11 | THY1 | 1.17 | CD.Plsma/IgA.IGHA2.IGKV1-12 | CCR10 | 0.38 |
| CD.EC.APOB.APOC3 | MME | 1.94 | CD.Fibro.FIGF.MMP11 | HTRA1 | 1.17 | CD.Plsma/IgA.IGKV1-12.IGHA2 | IGKV1-12 | 3.79 |
| CD.EC.APOB.APOC3 | CYP3A5 | 1.94 | CD.Fibro.FIGF.MMP11 | FKBP10 | 1.13 | CD.Plsma/IgA.IGKV1-12.IGHA2 | IGHA2 | 3.15 |
| CD.EC.APOB.APOC3 | CYP3A4 | 1.92 | CD.Fibro.FIGF.MMP11 | COL5A1 | 1.12 | CD.Plsma/IgA.IGKV1-12.IGHA2 | IGKV1-5 | 1.96 |
| CD.EC.APOB.APOC3 | FLJ22763 | 1.91 | CD.Fibro.FIGF.MMP11 | CCDC80 | 1.11 | CD.Plsma/IgA.IGKV1-12.IGHA2 | IGKV4-1 | 1.77 |
| CD.EC.APOB.APOC3 | CDHR2 | 1.89 | CD.Fibro.FIGF.MMP11 | ISLR | 1.11 | CD.Plsma/IgA.IGKV1-12.IGHA2 | MZB1 | 1.31 |
| CD.EC.APOB.APOC3 | DST | 1.87 | CD.Fibro.FIGF.MMP11 | LOXL2 | 1.08 | CD.Plsma/IgA.IGKV1-12.IGHA2 | DERL3 | 1.21 |
| CD.EC.APOB.APOC3 | TM4SF20 | 1.87 | CD.Fibro.FIGF.MMP11 | FENDRR | 1.05 | CD.Plsma/IgA.IGKV1-12.IGHA2 | TNFRSF17 | 1.13 |
| CD.EC.APOB.APOC3 | CES2 | 1.85 | CD.Fibro.FIGF.MMP11 | SRPX | 1.03 | CD.Plsma/IgA.IGKV1-12.IGHA2 | AL928768.3 | 0.84 |
| CD.EC.APOB.APOC3 | AOC1 | 1.83 | CD.Fibro.FIGF.MMP11 | HOXC9 | 0.99 | CD.Plsma/IgA.IGKV1-12.IGHA2 | IGKV3-11 | 1.01 |
| CD.EC.APOB.APOC3 | MGAM | 1.82 | CD.Fibro.FIGF.MMP11 | MXRA5 | 0.95 | CD.Plsma/IgA.IGKV1-12.IGHA2 | IGKV3-20 | 1.55 |
| CD.EC.APOB.APOC3 | ENPEP | 1.82 | CD.Fibro.FIGF.MMP11 | GLT8D2 | 0.94 | CD.Plsma/IgA.IGLC3.IGLC2 | AL928768.3 | 1.11 |
| CD.EC.APOB.APOC3 | KRT20 | 1.75 | CD.Fibro.FIGF.MMP11 | INSC | 0.91 | CD.Plsma/IgA.IGLC3.IGLC2 | DERL3 | 1.29 |
| CD.EC.APOB.APOC3 | SLC5A1 | 1.71 | CD.Fibro.FIGF.MMP11 | MAB21L2 | 0.90 | CD.Plsma/IgA.IGLC3.IGLC2.1 | IGLC2 | 3.61 |
| CD.EC.APOB.APOC3 | CREB3L3 | 1.69 | CD.Fibro.FIGF.MMP11 | LY6H | 0.89 | CD.Plsma/IgA.IGLC3.IGLC2.1 | IGLC3 | 3.33 |
| CD.EC.APOB.APOC3 | KHK | 1.67 | CD.Fibro.FIGF.MMP11 | CDH13 | 0.88 | CD.Plsma/IgA.IGLC3.IGLC2.1 | IGHA1 | 2.50 |
| CD.EC.APOB.APOC3 | SLC15A1 | 1.65 | CD.Fibro.FIGF.MMP11 | GLP2R | 0.87 | CD.Plsma/IgA.IGLC3.IGLC2.1 | IGHA2 | 2.49 |
| CD.EC.APOB.APOC3 | GDA | 1.63 | CD.Fibro.FIGF.MMP11 | PODN | 0.86 | CD.Plsma/IgA.IGLC3.IGLC2.1 | JCHAIN | 2.20 |
| CD.EC.APOB.APOC3 | MYO1A | 1.62 | CD.Fibro.FIGF.MMP11 | COL4A5 | 0.83 | CD.Plsma/IgA.IGLC3.IGLC2.1 | MZB1 | 1.71 |
| CD.EC.APOB.APOC3 | UGT2A3 | 1.61 | CD.Fibro.FIGF.MMP11 | SGCA | 0.82 | CD.Plsma/IgA.IGLC3.IGLC2.1 | DERL3 | 1.57 |
| CD.EC.APOB.APOC3 | GUCA2B | 1.59 | CD.Fibro.FIGF.MMP11 | PDE1A | 0.80 | CD.Plsma/IgA.IGLC3.IGLC2.1 | SSR4 | 1.45 |
| CD.EC.APOB.APOC3 | ACE2 | 1.57 | CD.Fibro.FIGF.MMP11 | HTRA3 | 0.78 | CD.Plsma/IgA.IGLC3.IGLC2.1 | FKBP11 | 1.21 |
| CD.EC.APOB.APOC3 | MEP1B | 1.55 | CD.Fibro.FIGF.MMP11 | PTGIS | 0.77 | CD.Plsma/IgA.IGLC3.IGLC2.1 | TNFRSF17 | 0.92 |
| CD.EC.APOB.APOC3 | MISP | 1.54 | CD.Fibro.FIGF.MMP11 | VSTM2A | 0.76 | CD.Plsma/IgA.IGLC3.IGLC2.1 | PRDX4 | 0.88 |
| CD.EC.APOB.APOC3 | HNF4G | 1.52 | CD.Fibro.FIGF.MMP11 | CNTFR | 0.57 | CD.Plsma/IgA.IGLC3.IGLC2.1 | IGLC6 | 0.78 |
| CD.EC.APOB.APOC3 | C11orf86 | 1.51 | CD.Fibro.FIGF.MMP11 | PTGDR2 | 0.55 | CD.Plsma/IgA.IGLC3.IGLC2.1 | AL928768.3 | 0.64 |
| CD.EC.APOB.APOC3 | MALL | 1.50 | CD.Fibro.FIGF.MMP11 | CPED1 | 1.05 | CD.Plsma/IgA.IGLC3.IGLC2.1 | XBP1 | 1.14 |
| CD.EC.APOB.APOC3 | TMIGD1 | 1.48 | CD.Fibro.FIGF.MMP11 | LHFP | 1.19 | CD.Plsma/IgA.IGLC3.IGLC2.1 | SEC11C | 0.81 |
| CD.EC.APOB.APOC3 | TMC5 | 1.46 | CD.Fibro.FIGF.MMP11 | CLEC11A | 1.38 | CD.Plsma/IgA.IGLC3.IGLC2.2 | MZB1 | 1.74 |
| CD.EC.APOB.APOC3 | SLC2A2 | 1.45 | CD.Fibro.FIGF.MMP11 | PPP1R14A | 1.33 | CD.Plsma/IgA.IGLC3.IGLC2.2 | AL928768.3 | 1.70 |
| CD.EC.APOB.APOC3 | MUC3A | 1.44 | CD.Fibro.FIGF.MMP11 | F2R | 1.24 | CD.Plsma/IgA.IGLC3.IGLC2.2 | DERL3 | 1.41 |
| CD.EC.APOB.APOC3 | PRSS8 | 1.43 | CD.Fibro.FIGF.MMP11 | WLS | 1.27 | CD.Plsma/IgA.IGLC3.IGLC2.2 | FKBP11 | 1.22 |
| CD.EC.APOB.APOC3 | LMO7 | 1.40 | CD.Fibro.FIGF.MMP11 | CLEC14A | 1.22 | CD.Plsma/IgA.IGLC3.IGLC2.2 | TNFRSF17 | 1.03 |
| CD.EC.APOB.APOC3 | GNA11 | 1.40 | CD.Fibro.FIGF.MMP11 | COL4A6 | 0.49 | CD.Plsma/IgA.IGLC3.IGLC2.2 | BEX5 | 0.34 |
| CD.EC.APOB.APOC3 | SLC5A12 | 1.40 | CD.Fibro.FIGF.MMP11 | LTBP4 | 1.57 | CD.Plsma/IgA.IGLC3.IGLC2.2 | DUSP5 | 0.89 |
| CD.EC.APOB.APOC3 | CPO | 1.39 | CD.Fibro.FIGF.MMP11 | CXCL12 | 1.41 | CD.Plsma/IgA.IGLC3.IGLC2.2 | SEC11C | 1.12 |
| CD.EC.APOB.APOC3 | SLC10A2 | 1.38 | CD.Fibro.FIGF.MMP11 | PLAU | 1.28 | CD.Plsma/IgA.IGLC3.IGLC2.2 | CCR10 | 0.45 |
| CD.EC.APOB.APOC3 | XPNPEP2 | 1.37 | CD.Fibro.FIGF.MMP11 | C11orf96 | 1.14 | CD.Plsma/IgA.IGLL5.IGLV2-14 | IGLL5 | 4.45 |
| CD.EC.APOB.APOC3 | SEMA3B | 1.35 | CD.Fibro.FIGF.MMP11 | IGFBP3 | 1.48 | CD.Plsma/IgA.IGLL5.IGLV2-14 | IGHA1 | 2.94 |
| CD.EC.APOB.APOC3 | SULT1A2 | 1.35 | CD.Fibro.FIGF.MMP11 | EMID1 | 0.88 | CD.Plsma/IgA.IGLL5.IGLV2-14 | IGHA2 | 2.76 |
| CD.EC.APOB.APOC3 | UGT2B7 | 1.33 | CD.Fibro.FIGF.MMP11 | TFPI | 1.28 | CD.Plsma/IgA.IGLL5.IGLV2-14 | JCHAIN | 2.73 |
| CD.EC.APOB.APOC3 | ABCG2 | 1.31 | CD.Fibro.FIGF.MMP11 | MOXD1 | 1.06 | CD.Plsma/IgA.IGLL5.IGLV2-14 | IGLV2-14 | 2.25 |
| CD.EC.APOB.APOC3 | TBX3 | 1.29 | CD.Fibro.FIGF.MMP11 | SRPX2 | 0.62 | CD.Plsma/IgA.IGLL5.IGLV2-14 | MZB1 | 2.09 |
| CD.EC.APOB.APOC3 | ALPI | 1.27 | CD.Fibro.FIGF.MMP11 | NKX2-3 | 1.05 | CD.Plsma/IgA.IGLL5.IGLV2-14 | SSR4 | 1.91 |
| CD.EC.APOB.APOC3 | SLC6A8 | 1.27 | CD.Fibro.GDF15.EGR1 | AGT | 2.00 | CD.Plsma/IgA.IGLL5.IGLV2-14 | DERL3 | 1.86 |
| CD.EC.APOB.APOC3 | REEP6 | 1.24 | CD.Fibro.HIGD1B.RGS5 | RGS5 | 4.27 | CD.Plsma/IgA.IGLL5.IGLV2-14 | FKBP11 | 1.63 |
| CD.EC.APOB.APOC3 | MS4A8 | 1.23 | CD.Fibro.HIGD1B.RGS5 | IGFBP7 | 3.27 | CD.Plsma/IgA.IGLL5.IGLV2-14 | AL928768.3 | 1.56 |
| CD.EC.APOB.APOC3 | MOGAT2 | 1.23 | CD.Fibro.HIGD1B.RGS5 | CALD1 | 3.07 | CD.Plsma/IgA.IGLL5.IGLV2-14 | TNFRSF17 | 1.49 |
| CD.EC.APOB.APOC3 | SLC3A1 | 1.20 | CD.Fibro.HIGD1B.RGS5 | ACTA2 | 2.96 | CD.Plsma/IgA.IGLL5.IGLV2-14 | SEC11C | 1.43 |
| CD.EC.APOB.APOC3 | SCIN | 1.20 | CD.Fibro.HIGD1B.RGS5 | MYL9 | 2.78 | CD.Plsma/IgA.IGLL5.IGLV2-14 | PRDX4 | 1.22 |
| CD.EC.APOB.APOC3 | TM4SF5 | 1.20 | CD.Fibro.HIGD1B.RGS5 | MGP | 2.64 | CD.Plsma/IgA.IGLL5.IGLV2-14 | ITM2C | 1.19 |
| CD.EC.APOB.APOC3 | GPA33 | 1.18 | CD.Fibro.HIGD1B.RGS5 | SPARC | 2.60 | CD.Plsma/IgA.IGLL5.IGLV2-14 | CCR10 | 0.64 |
| CD.EC.APOB.APOC3 | SULT1B1 | 1.17 | CD.Fibro.HIGD1B.RGS5 | NDUFA4L2 | 2.45 | CD.Plsma/IgA.IGLL5.IGLV2-14 | IGLC7 | 0.41 |
| CD.EC.APOB.APOC3 | SLC51B | 1.17 | CD.Fibro.HIGD1B.RGS5 | SOD3 | 2.45 | CD.Plsma/IgA.IGLL5.IGLV2-14 | IGLL1 | 0.38 |
| CD.EC.APOB.APOC3 | LINC01133 | 1.15 | CD.Fibro.HIGD1B.RGS5 | COL3A1 | 2.25 | CD.Plsma/IgA.IGLL5.IGLV2-14 | DUSP5 | 1.02 |
| CD.EC.APOB.APOC3 | ZG16 | 1.10 | CD.Fibro.HIGD1B.RGS5 | TAGLN | 2.21 | CD.Plsma/IgA.IGLL5.IGLV2-14 | XBP1 | 1.37 |
| CD.EC.APOB.APOC3 | SLC13A1 | 1.10 | CD.Fibro.HIGD1B.RGS5 | CAV1 | 2.19 | CD.Plsma/IgA.IGLL5.IGLV2-14 | ANKRD28 | 0.85 |
| CD.EC.APOB.APOC3 | ENPP7 | 1.09 | CD.Fibro.HIGD1B.RGS5 | ADIRF | 2.18 | CD.Plsma/IgA.IGLL5.IGLV2-14 | MANF | 0.99 |
| CD.EC.APOB.APOC3 | MFSD2A | 1.07 | CD.Fibro.HIGD1B.RGS5 | COL1A2 | 2.15 | CD.Plsma/IgA.IGLL5.IGLV2-14 | CRELD2 | 0.84 |
| CD.EC.APOB.APOC3 | HHLA2 | 1.06 | CD.Fibro.HIGD1B.RGS5 | FRZB | 2.15 | CD.Plsma/IgA.IGLL5.IGLV2-14 | SSR3 | 1.00 |
| CD.EC.APOB.APOC3 | TREH | 1.06 | CD.Fibro.HIGD1B.RGS5 | NOTCH3 | 2.06 | CD.Plsma/IgA.IGLL5.IGLV2-14 | UBE2J1 | 0.99 |
| CD.EC.APOB.APOC3 | MYO15B | 1.05 | CD.Fibro.HIGD1B.RGS5 | TPM2 | 2.05 | CD.Plsma/IgA.IGLL5.IGLV2-14 | VIMP | 1.05 |
| CD.EC.APOB.APOC3 | CIDEC | 1.03 | CD.Fibro.HIGD1B.RGS5 | COX412 | 2.05 | CD.Plsma/IgA.IGLV1-51.IGLV10-54 | IGLV6-57 | 3.18 |
| CD.EC.APOB.APOC3 | SLC7A9 | 0.97 | CD.Fibro.HIGD1B.RGS5 | STEAP4 | 2.00 | CD.Plsma/IgA.IGLV1-51.IGLV10-54 | IGLV10-54 | 3.02 |
| CD.EC.APOB.APOC3 | SUSD2 | 0.93 | CD.Fibro.HIGD1B.RGS5 | COL4A1 | 2.00 | CD.Plsma/IgA.IGLV1-51.IGLV10-54 | IGLV1-51 | 2.68 |
| CD.EC.APOB.APOC3 | TM6SF2 | 0.93 | CD.Fibro.HIGD1B.RGS5 | COL1A1 | 1.98 | CD.Plsma/IgA.IGLV1-51.IGLV10-54 | MZB1 | 1.72 |
| CD.EC.APOB.APOC3 | RP11- | 0.93 | CD.Fibro.HIGD1B.RGS5 | COL4A2 | 1.94 | CD.Plsma/IgA.IGLV1-51.IGLV10-54 | DERL3 | 1.58 |
| | 462G2.1 | | | | | | | |
| CD.EC.APOB.APOC3 | CDKN2B-AS1 | 0.92 | CD.Fibro.HIGD1B.RGS5 | ASPN | 1.92 | CD.Plsma/IgA.IGLV1-51.IGLV10-54 | AL928768.3 | 0.89 |
| CD.EC.APOB.APOC3 | SLC1A1 | 0.91 | CD.Fibro.HIGD1B.RGS5 | THY1 | 1.90 | CD.Plsma/IgA.IGLV1-51.IGLV10-54 | TNFRSF17 | 0.96 |
| CD.EC.APOB.APOC3 | FOLH1 | 0.89 | CD.Fibro.HIGD1B.RGS5 | COL18A1 | 1.90 | CD.Plsma/IgA.IGLV2-14.IGLV3-19 | IGLV2-14 | 3.10 |
| CD.EC.APOB.APOC3 | AADAC | 0.81 | CD.Fibro.HIGD1B.RGS5 | HIGD1B | 1.90 | CD.Plsma/IgA.IGLV2-14.IGLV3-19 | IGLV2-23 | 1.96 |
| CD.EC.APOB.APOC3 | MS4A10 | 0.78 | CD.Fibro.HIGD1B.RGS5 | PDGFRB | 1.88 | CD.Plsma/IgA.IGLV2-14.IGLV3-19 | IGLV2-11 | 1.95 |
| CD.EC.APOB.APOC3 | CYP2C18 | 0.78 | CD.Fibro.HIGD1B.RGS5 | CD36 | 1.87 | CD.Plsma/IgA.IGLV2-14.IGLV3-19 | IGLV2-8 | 1.87 |
| CD.EC.APOB.APOC3 | CYP2C9 | 0.77 | CD.Fibro.HIGD1B.RGS5 | TFPI | 1.83 | CD.Plsma/IgA.IGLV2-14.IGLV3-19 | IGLV3-19 | 1.75 |
| CD.EC.APOB.APOC3 | SLC6A4 | 0.77 | CD.Fibro.HIGD1B.RGS5 | NR2F2 | 1.82 | CD.Plsma/IgA.IGLV2-14.IGLV3-19 | IGLV1-47 | 1.61 |
| CD.EC.APOB.APOC3 | SLC51A | 0.77 | CD.Fibro.HIGD1B.RGS5 | LHFP | 1.82 | CD.Plsma/IgA.IGLV2-14.IGLV3-19 | IGLV1-51 | 0.59 |
| CD.EC.APOB.APOC3 | SLC23A1 | 0.77 | CD.Fibro.HIGD1B.RGS5 | C1R | 1.80 | CD.Plsma/IgA.IGLV2-14.IGLV3-19 | IGLV2-18 | 0.99 |
| CD.EC.APOB.APOC3 | ERICH4 | 0.74 | CD.Fibro.HIGD1B.RGS5 | C1S | 1.76 | CD.Plsma/IgA.MT-ND5.MT-ND4L | IGHA1 | 2.62 |
| CD.EC.APOB.APOC3 | VNN1 | 0.72 | CD.Fibro.HIGD1B.RGS5 | EPAS1 | 1.76 | CD.Plsma/IgA.MT-ND5.MT-ND4L | MZB1 | 1.71 |
| CD.EC.APOB.APOC3 | PLB1 | 0.67 | CD.Fibro.HIGD1B.RGS5 | CPE | 1.76 | CD.Plsma/IgA.MT-ND5.MT-ND4L | JCHAIN | 1.54 |
| CD.EC.APOB.APOC3 | SULT2A1 | 0.64 | CD.Fibro.HIGD1B.RGS5 | MFGE8 | 1.74 | CD.Plsma/IgA.MT-ND5.MT-ND4L | IGKC | 1.81 |
| CD.EC.APOB.APOC3 | SMLR1 | 0.63 | CD.Fibro.HIGD1B.RGS5 | BGN | 1.66 | CD.Plsma/IgA.RRM2.TOP2A | RRM2 | 1.22 |
| CD.EC.APOB.APOC3 | PTPRR | 0.61 | CD.Fibro.HIGD1B.RGS5 | TPPP3 | 1.64 | CD.Plsma/IgA.RRM2.TOP2A | BUB1 | 0.60 |
| CD.EC.APOB.APOC3 | ABCC2 | 0.58 | CD.Fibro.HIGD1B.RGS5 | PRSS23 | 1.63 | CD.Plsma/IgA.RRM2.TOP2A | ASPM | 0.71 |
| CD.EC.APOB.APOC3 | DAB1 | 0.58 | CD.Fibro.HIGD1B.RGS5 | CSRP2 | 1.60 | CD.Plsma/IgG.IGHGP.IGHG2 | IGHG1 | 5.29 |
| CD.EC.APOB.APOC3 | AQP7 | 0.57 | CD.Fibro.HIGD1B.RGS5 | TINAGL1 | 1.58 | CD.Plsma/IgG.IGHGP.IGHG2 | IGHG3 | 5.22 |
| CD.EC.APOB.APOC3 | SLC17A8 | 0.56 | CD.Fibro.HIGD1B.RGS5 | SYNPO2 | 1.52 | CD.Plsma/IgG.IGHGP.IGHG2 | IGHG2 | 4.79 |
| CD.EC.APOB.APOC3 | DPP4 | 1.31 | CD.Fibro.HIGD1B.RGS5 | MAP1B | 1.49 | CD.Plsma/IgG.IGHGP.IGHG2 | IGHG4 | 4.77 |
| CD.EC.APOB.APOC3 | CYP4F2 | 0.64 | CD.Fibro.HIGD1B.RGS5 | COX7A1 | 1.49 | CD.Plsma/IgG.IGHGP.IGHG2 | IGHGP | 3.22 |
| CD.EC.APOB.APOC3 | GPD1 | 0.89 | CD.Fibro.HIGD1B.RGS5 | RARRES2 | 1.49 | CD.Plsma/IgG.IGHGP.IGHG2 | IGKC | 2.33 |
| CD.EC.APOB.APOC3 | CYP2J2 | 0.76 | CD.Fibro.HIGD1B.RGS5 | CCDC102B | 1.39 | CD.Plsma/IgG.IGHGP.IGHG2 | MZB1 | 2.02 |
| CD.EC.APOB.APOC3 | FLVCR1 | 1.17 | CD.Fibro.HIGD1B.RGS5 | 4-Sep | 1.36 | CD.Plsma/IgG.IGHGP.IGHG2 | XBP1 | 1.74 |
| CD.EC.APOB.APOC3 | C1orf115 | 0.93 | CD.Fibro.HIGD1B.RGS5 | ADGRF5 | 1.30 | CD.Plsma/IgG.IGHGP.IGHG2 | SSR4 | 1.57 |
| CD.EC.APOB.APOC3 | C19orf33 | 1.62 | CD.Fibro.HIGD1B.RGS5 | SMOC2 | 1.28 | CD.Plsma/IgG.IGHGP.IGHG2 | IGLV6-57 | 1.39 |
| CD.EC.APOB.APOC3 | FAM132A | 0.77 | CD.Fibro.HIGD1B.RGS5 | ARHGAP29 | 1.26 | CD.Plsma/IgG.IGHGP.IGHG2 | DERL3 | 1.23 |
| CD.EC.APOB.APOC3 | C8G | 0.64 | CD.Fibro.HIGD1B.RGS5 | EGFL6 | 1.22 | CD.Plsma/IgG.IGHGP.IGHG2 | FKBP11 | 1.17 |
| CD.EC.APOB.APOC3 | ESPN | 1.02 | CD.Fibro.HIGD1B.RGS5 | EDNRA | 1.14 | CD.Plsma/IgG.IGHGP.IGHG2 | PRDX4 | 1.17 |
| CD.EC.APOB.APOC3 | PCSK5 | 0.88 | CD.Fibro.HIGD1B.RGS5 | FAM13C | 1.10 | CD.Plsma/IgG.IGHGP.IGHG2 | HERPUD1 | 0.98 |
| CD.EC.APOC3.APOA4 | APOA1 | 4.38 | CD.Fibro.HIGD1B.RGS5 | GJA4 | 1.09 | CD.Plsma/IgG.IGHGP.IGHG2 | IGLV3-1 | 0.95 |
| CD.EC.APOC3.APOA4 | FABP6 | 4.32 | CD.Fibro.HIGD1B.RGS5 | FAM162B | 1.09 | CD.Plsma/IgG.IGHGP.IGHG2 | SEC11C | 0.95 |
| CD.EC.APOC3.APOA4 | APOA4 | 4.21 | CD.Fibro.HIGD1B.RGS5 | KCNJ8 | 1.05 | CD.Plsma/IgG.IGHGP.IGHG2 | ITM2C | 0.94 |
| CD.EC.APOC3.APOA4 | APOC3 | 3.99 | CD.Fibro.HIGD1B.RGS5 | GUCY1B3 | 1.03 | CD.Plsma/IgG.IGHGP.IGHG2 | HSP90B1 | 0.85 |
| CD.EC.APOC3.APOA4 | FABP1 | 3.59 | CD.Fibro.HIGD1B.RGS5 | FOXS1 | 1.02 | CD.Plsma/IgG.IGHGP.IGHG2 | JUN | 0.82 |
| CD.EC.APOC3.APOA4 | GUCAZA | 3.16 | CD.Fibro.HIGD1B.RGS5 | MCAM | 1.00 | CD.Plsma/IgG.IGHGP.IGHG2 | JCHAIN | 0.74 |
| CD.EC.APOC3.APOA4 | APOB | 2.88 | CD.Fibro.HIGD1B.RGS5 | ABCC9 | 0.93 | CD.Plsma/IgG.IGHGP.IGHG2 | PIM2 | 0.73 |
| CD.EC.APOC3.APOA4 | RBP2 | 2.75 | CD.Fibro.HIGD1B.RGS5 | GJC1 | 0.92 | CD.Plsma/IgG.IGHGP.IGHG2 | VIMP | 0.70 |
| CD.EC.APOC3.APOA4 | CDHR5 | 2.47 | CD.Fibro.HIGD1B.RGS5 | REM1 | 0.84 | CD.Plsma/IgG.IGHGP.IGHG2 | FKBP2 | 0.68 |
| CD.EC.APOC3.APOA4 | GUCA2B | 2.31 | CD.Fibro.HIGD1B.RGS5 | LG14 | 0.83 | CD.Plsma/IgG.IGHGP.IGHG2 | DUSP5 | 0.67 |
| CD.EC.APOC3.APOA4 | SLC26A3 | 2.14 | CD.Fibro.HIGD1B.RGS5 | GUCY1A2 | 0.82 | CD.Plsma/IgG.IGHGP.IGHG2 | SPCS3 | 0.66 |
| CD.EC.APOC3.APOA4 | MTTP | 2.06 | CD.Fibro.HIGD1B.RGS5 | GPX3 | 1.54 | CD.Plsma/IgG.IGHGP.IGHG2 | SSR3 | 0.64 |
| CD.EC.APOC3.APOA4 | CUBN | 1.94 | CD.Fibro.HIGD1B.RGS5 | RCAN2 | 0.99 | CD.Plsma/IgG.IGHGP.IGHG2 | DNAJB9 | 0.64 |
| CD.EC.APOC3.APOA4 | FLJ22763 | 1.79 | CD.Fibro.HIGD1B.RGS5 | PTRF | 1.42 | CD.Plsma/IgG.IGHGP.IGHG2 | ERLEC1 | 0.63 |
| CD.EC.APOC3.APOA4 | C11orf86 | 1.51 | CD.Fibro.HIGD1B.RGS5 | MYLK | 1.47 | CD.Plsma/IgG.IGHGP.IGHG2 | MYDGF | 0.63 |
| CD.EC.APOC3.APOA4 | SLC6A19 | 1.48 | CD.Fibro.HIGD1B.RGS5 | CDH6 | 0.76 | CD.Plsma/IgG.IGHGP.IGHG2 | SPCS2 | 0.62 |
| CD.EC.APOC3.APOA4 | CDHR2 | 1.41 | CD.Fibro.HIGD1B.RGS5 | FHL5 | 0.70 | CD.Plsma/IgG.IGHGP.IGHG2 | ANKRD28 | 0.59 |
| CD.EC.APOC3.APOA4 | MGAM | 1.40 | CD.Fibro.HIGD1B.RGS5 | GUCY1A3 | 1.16 | CD.Plsma/IgG.IGHGP.IGHG2 | JSRP1 | 0.59 |
| CD.EC.APOC3.APOA4 | CYP3A4 | 1.32 | CD.Fibro.HIGD1B.RGS5 | SERPING1 | 1.88 | CD.Plsma/IgG.IGHGP.IGHG2 | TNFRSF17 | 0.58 |
| CD.EC.APOC3.APOA4 | CPO | 1.15 | CD.Fibro.HIGD1B.RGS5 | ECM2 | 0.90 | CD.Plsma/IgG.IGHGP.IGHG2 | SDC1 | 0.55 |
| CD.EC.APOC3.APOA4 | SLC15A1 | 0.99 | CD.Fibro.HIGD1B.RGS5 | PRKCDBP | 1.54 | CD.Plsma/IgG.IGHGP.IGHG2 | CD38 | 0.53 |
| CD.EC.APOC3.APOA4 | ANPEP | 2.58 | CD.Fibro.HIGD1B.RGS5 | SPARCL1 | 1.60 | CD.Plsma/IgG.IGHGP.IGHG2 | MANF | 0.51 |
| CD.EC.APOC3.APOA4 | PCK1 | 2.05 | CD.Fibro.HIGD1B.RGS5 | COL5A2 | 1.25 | CD.Plsma/IgG.IGHGP.IGHG2 | LMAN1 | 0.50 |
| CD.EC.APOC3.APOA4 | FABP2 | 3.45 | CD.Fibro.HIGD1B.RGS5 | MYO1B | 1.37 | CD.Plsma/IgG.IGHGP.IGHG2 | SDF2L1 | 0.50 |
| CD.EC.APOC3.APOA4 | CREB3L3 | 1.61 | CD.Fibro.IFI6.IFI44L | TDO2 | 1.73 | CD.Plsma/IgG.IGHGP.IGHG2 | TXNDC15 | 0.49 |
| CD.EC.APOC3.APOA4 | ALDOB | 3.18 | CD.Fibro.IGF2.CXCL6 | CXCL6 | 3.61 | CD.Plsma/IgG.IGHGP.IGHG2 | SEL1L | 0.49 |
| CD.EC.APOC3.CUBN | APOA1 | 4.95 | CD.Fibro.IGF2.CXCL6 | CP | 2.04 | CD.Plsma/IgG.IGHGP.IGHG2 | FCRL5 | 0.46 |
| CD.EC.APOC3.CUBN | APOA4 | 4.53 | CD.Fibro.IGF2.CXCL6 | COL14A1 | 1.85 | CD.Plsma/IgG.IGHGP.IGHG2 | UBE2J1 | 0.44 |
| CD.EC.APOC3.CUBN | APOC3 | 4.27 | CD.Fibro.IGF2.CXCL6 | IGF2 | 1.29 | CD.Plsma/IgG.IGHGP.IGHG2 | CRELD2 | 0.44 |
| CD.EC.APOC3.CUBN | FABP6 | 4.24 | CD.Fibro.LY6H.PAPPA2 | APOD | 3.70 | CD.Plsma/IgG.IGHGP.IGHG2 | PLPP5 | 0.43 |
| CD.EC.APOC3.CUBN | PRAP1 | 3.68 | CD.Fibro.LY6H.PAPPA2 | CXCL14 | 3.67 | CD.Plsma/IgG.IGHGP.IGHG2 | CD27 | 0.43 |
| CD.EC.APOC3.CUBN | PHGR1 | 3.64 | CD.Fibro.LY6H.PAPPA2 | IGFBP5 | 3.52 | CD.Plsma/IgG.IGHGP.IGHG2 | PDIA4 | 0.42 |
| CD.EC.APOC3.CUBN | ADIRF | 3.30 | CD.Fibro.LY6H.PAPPA2 | CALD1 | 3.30 | CD.Plsma/IgG.IGHGP.IGHG2 | CD79A | 0.41 |
| CD.EC.APOC3.CUBN | FABP1 | 3.20 | CD.Fibro.LY6H.PAPPA2 | TAGLN | 3.27 | CD.Plsma/IgG.IGHGP.IGHG2 | CADM1 | 0.41 |
| CD.EC.APOC3.CUBN | APOB | 3.12 | CD.Fibro.LY6H.PAPPA2 | ACTA2 | 3.19 | CD.Plsma/IgG.IGHGP.IGHG2 | TXNDC11 | 0.41 |
| CD.EC.APOC3.CUBN | FABP2 | 3.02 | CD.Fibro.LY6H.PAPPA2 | ADH1B | 2.91 | CD.Plsma/IgG.IGHGP.IGHG2 | PRDM1 | 0.41 |
| CD.EC.APOC3.CUBN | GUCAZA | 2.92 | CD.Fibro.LY6H.PAPPA2 | CTGF | 2.86 | CD.Plsma/IgG.IGHGP.IGHG2 | FAM46C | 0.40 |
| CD.EC.APOC3.CUBN | ALDOB | 2.85 | CD.Fibro.LY6H.PAPPA2 | MMP1 | 2.72 | CD.Plsma/IgG.IGHGP.IGHG2 | TRIB1 | 0.40 |
| CD.EC.APOC3.CUBN | DPEP1 | 2.75 | CD.Fibro.LY6H.PAPPA2 | RARRES2 | 2.67 | CD.Plsma/IgG.IGHGP.IGHG2 | SPAG4 | 0.40 |
| CD.EC.APOC3.CUBN | CDHR5 | 2.66 | CD.Fibro.LY6H.PAPPA2 | C1R | 2.60 | CD.Plsma/IgG.IGHGP.IGHG2 | RPN2 | 0.38 |
| CD.EC.APOC3.CUBN | ANPEP | 2.60 | CD.Fibro.LY6H.PAPPA2 | C15 | 2.60 | CD.Plsma/IgG.IGHGP.IGHG2 | LINC00152 | 0.35 |
| CD.EC.APOC3.CUBN | SLC26A3 | 2.54 | CD.Fibro.LY6H.PAPPA2 | EDNRB | 2.55 | CD.Plsma/IgG.IGHGP.IGHG2 | TRAM1 | 0.33 |
| CD.EC.APOC3.CUBN | CUBN | 2.50 | CD.Fibro.LY6H.PAPPA2 | TPM2 | 2.50 | CD.Plsma/IgG.IGHGP.IGHG2 | ZBP1 | 0.33 |
| CD.EC.APOC3.CUBN | RBP2 | 2.45 | CD.Fibro.LY6H.PAPPA2 | PDGFRA | 2.46 | CD.Plsma/IgG.IGHGP.IGHG2 | KDELR2 | 0.33 |
| CD.EC.APOC3.CUBN | AMN | 2.39 | CD.Fibro.LY6H.PAPPA2 | COL3A1 | 2.38 | CD.Plsma/IgG.IGHGP.IGHG2 | HM13 | 0.33 |
| CD.EC.APOC3.CUBN | MTTP | 2.33 | CD.Fibro.LY6H.PAPPA2 | F3 | 2.32 | CD.Plsma/IgG.IGHGP.IGHG2 | TMED9 | 0.32 |
| CD.EC.APOC3.CUBN | C19orf33 | 2.26 | CD.Fibro.LY6H.PAPPA2 | COL6A2 | 2.30 | CD.Plsma/IgG.IGHGP.IGHG2 | ARSA | 0.32 |
| CD.EC.APOC3.CUBN | SMIM24 | 2.25 | CD.Fibro.LY6H.PAPPA2 | MYL9 | 2.17 | CD.Plsma/IgG.IGHGP.IGHG2 | RABAC1 | 0.31 |
| CD.EC.APOC3.CUBN | SERPINA1 | 2.22 | CD.Fibro.LY6H.PAPPA2 | COL1A2 | 2.15 | CD.Plsma/IgG.IGHGP.IGHG2 | RP11- | 0.31 |
| | | | | | | | 16E12.2 | |
| CD.EC.APOC3.CUBN | CYP3A5 | 2.14 | CD.Fibro.LY6H.PAPPA2 | SOD3 | 2.15 | CD.Plsma/IgG.IGHGP.IGHG2 | ELL2 | 0.31 |
| CD.EC.APOC3.CUBN | DST | 2.11 | CD.Fibro.LY6H.PAPPA2 | MMP2 | 2.14 | CD.Plsma/IgG.IGHGP.IGHG2 | PDK1 | 0.30 |
| CD.EC.APOC3.CUBN | GUCA2B | 2.07 | CD.Fibro.LY6H.PAPPA2 | COL6A1 | 2.06 | CD.Plsma/IgG.IGHGP.IGHG2 | SLAMF7 | 0.30 |
| CD.EC.APOC3.CUBN | PLS1 | 2.04 | CD.Fibro.LY6H.PAPPA2 | ACTG2 | 2.06 | CD.Plsma/IgG.IGHGP.IGHG2 | GAS6 | 0.30 |
| CD.EC.APOC3.CUBN | PCK1 | 2.04 | CD.Fibro.LY6H.PAPPA2 | LY6H | 2.05 | CD.Plsma/IgG.IGHGP.IGHG2 | CHPF | 0.30 |
| CD.EC.APOC3.CUBN | KRT20 | 2.01 | CD.Fibro.LY6H.PAPPA2 | SULF1 | 2.03 | CD.Plsma/IgG.IGHGP.IGHG2 | EAF2 | 0.29 |
| CD.EC.APOC3.CUBN | SEPP1 | 1.99 | CD.Fibro.LY6H.PAPPA2 | COL1A1 | 2.00 | CD.Plsma/IgG.IGHGP.IGHG2 | ALG5 | 0.29 |
| CD.EC.APOC3.CUBN | ARL14 | 1.99 | CD.Fibro.LY6H.PAPPA2 | MFAP4 | 1.98 | CD.Plsma/IgG.IGHGP.IGHG2 | RRBP1 | 0.29 |
| CD.EC.APOC3.CUBN | ACE | 1.94 | CD.Fibro.LY6H.PAPPA2 | BMP4 | 1.77 | CD.Plsma/IgG.IGHGP.IGHG2 | SELM | 0.28 |
| CD.EC.APOC3.CUBN | AGPAT2 | 1.92 | CD.Fibro.LY6H.PAPPA2 | MMP3 | 1.76 | CD.Plsma/IgG.IGHGP.IGHG2 | PECAM1 | 0.28 |
| CD.EC.APOC3.CUBN | PLCG2 | 1.90 | CD.Fibro.LY6H.PAPPA2 | OLFML3 | 1.74 | CD.Plsma/IgG.IGHGP.IGHG2 | POU2AF1 | 0.28 |
| CD.EC.APOC3.CUBN | MUC13 | 1.83 | CD.Fibro.LY6H.PAPPA2 | LAMA4 | 1.71 | CD.Plsma/IgG.IGHGP.IGHG2 | TPD52 | 0.27 |
| CD.EC.APOC3.CUBN | ACSL5 | 1.83 | CD.Fibro.LY6H.PAPPA2 | FN1 | 1.71 | CD.Plsma/IgG.IGHGP.IGHG2 | SPATS2 | 0.26 |
| CD.EC.APOC3.CUBN | DGAT1 | 1.82 | CD.Fibro.LY6H.PAPPA2 | LIMCH1 | 1.65 | CD.Plsma/IgG.IGHGP.IGHG2 | TMED10 | 0.26 |
| CD.EC.APOC3.CUBN | CLDN4 | 1.81 | CD.Fibro.LY6H.PAPPA2 | NSG1 | 1.59 | CD.Plsma/IgG.IGHGP.IGHG2 | CCPG1 | 0.26 |
| CD.EC.APOC3.CUBN | LMO7 | 1.73 | CD.Fibro.LY6H.PAPPA2 | ALDH1A3 | 1.59 | CD.Plsma/IgG.IGHGP.IGHG2 | MANEA | 0.25 |
| CD.EC.APOC3.CUBN | C11orf86 | 1.70 | CD.Fibro.LY6H.PAPPA2 | AGT | 1.59 | CD.Plsma/IgG.IGHGP.IGHG2 | DNAJC1 | 0.25 |
| CD.EC.APOC3.CUBN | TM4SF20 | 1.68 | CD.Fibro.LY6H.PAPPA2 | PCOLCE | 1.55 | CD.Plsma/IgG.IGHGP.IGHG2 | LMAN2 | 0.29 |
| CD.EC.APOC3.CUBN | ELF3 | 1.66 | CD.Fibro.LY6H.PAPPA2 | PTGIS | 1.54 | CD.Plsma/IgG.IGHGP.IGHG2 | MEI1 | 0.26 |
| CD.EC.APOC3.CUBN | FLJ22763 | 1.66 | CD.Fibro.LY6H.PAPPA2 | FRZB | 1.53 | CD.Plsma/IgG.IGHGP.IGHG2 | SRPRB | 0.26 |
| CD.EC.APOC3.CUBN | CDHR2 | 1.65 | CD.Fibro.LY6H.PAPPA2 | SDC2 | 1.50 | CD.Plsma/IgG.IGHGP.IGHG2 | SIL1 | 0.25 |
| CD.EC.APOC3.CUBN | TMC5 | 1.64 | CD.Fibro.LY6H.PAPPA2 | BMP5 | 1.46 | CD.Plsma/IgG.IGHGP.IGHG2 | COBLL1 | 0.26 |
| CD.EC.APOC3.CUBN | MME | 1.62 | CD.Fibro.LY6H.PAPPA2 | TRPA1 | 1.46 | CD.Plsma/IgG.IGHGP.IGHG2 | BHLHE41 | 0.26 |
| CD.EC.APOC3.CUBN | MTRNR2L8 | 1.61 | CD.Fibro.LY6H.PAPPA2 | EMILIN1 | 1.45 | CD.Plsma/IgG.IGHGP.IGHG2 | PDIA6 | 0.35 |
| CD.EC.APOC3.CUBN | SLC6A19 | 1.54 | CD.Fibro.LY6H.PAPPA2 | COL12A1 | 1.42 | CD.Plsma/IgG.IGHGP.IGHG2 | TMEM59 | 0.42 |
| CD.EC.APOC3.CUBN | GDA | 1.53 | CD.Fibro.LY6H.PAPPA2 | FIGF | 1.38 | CD.Plsma/IgG.IGHGP.IGHG2 | HIST1H2BG | 0.49 |
| CD.EC.APOC3.CUBN | AOC1 | 1.52 | CD.Fibro.LY6H.PAPPA2 | FAM150B | 1.37 | CD.Plsma/IgM.IGHM.IGLV2-14 | IGHM | 4.21 |
| CD.EC.APOC3.CUBN | MYO1A | 1.52 | CD.Fibro.LY6H.PAPPA2 | PDPN | 1.34 | CD.Plsma/IgM.IGHM.IGLV2-14 | IGLL5 | 2.63 |
| CD.EC.APOC3.CUBN | MT-ATP8 | 1.51 | CD.Fibro.LY6H.PAPPA2 | PDGFD | 1.32 | CD.Plsma/IgM.IGHM.IGLV2-14 | JCHAIN | 2.40 |
| CD.EC.APOC3.CUBN | KHK | 1.51 | CD.Fibro.LY6H.PAPPA2 | BGN | 1.31 | CD.Plsma/IgM.IGHM.IGLV2-14 | IGLV2-14 | 2.21 |
| CD.EC.APOC3.CUBN | EPS8L2 | 1.47 | CD.Fibro.LY6H.PAPPA2 | TMEM119 | 1.29 | CD.Plsma/IgM.IGHM.IGLV2-14 | MZB1 | 1.62 |
| CD.EC.APOC3.CUBN | ANGPTL4 | 1.45 | CD.Fibro.LY6H.PAPPA2 | CARMN | 1.25 | CD.Plsma/IgM.IGHM.IGLV2-14 | SSR4 | 1.42 |
| CD.EC.APOC3.CUBN | CYP3A4 | 1.45 | CD.Fibro.LY6H.PAPPA2 | PCSK6 | 1.22 | CD.Plsma/IgM.IGHM.IGLV2-14 | IGLC7 | 1.25 |
| CD.EC.APOC3.CUBN | CREB3L3 | 1.44 | CD.Fibro.LY6H.PAPPA2 | MRVI1 | 1.02 | CD.Plsma/IgM.IGHM.IGLV2-14 | DERL3 | 1.23 |
| CD.EC.APOC3.CUBN | SLC5A1 | 1.44 | CD.Fibro.LY6H.PAPPA2 | PAPPA2 | 0.93 | CD.Plsma/IgM.IGHM.IGLV2-14 | FKBP11 | 1.19 |
| CD.EC.APOC3.CUBN | SULT1A2 | 1.39 | CD.Fibro.LY6H.PAPPA2 | PODN | 0.80 | CD.Plsma/IgM.IGHM.IGLV2-14 | TNFRSF17 | 1.17 |
| CD.EC.APOC3.CUBN | MISP | 1.38 | CD.Fibro.LY6H.PAPPA2 | RP11- | 0.89 | CD.Plsma/IgM.IGHM.IGLV2-14 | IGLC3 | 3.02 |
| | | | | 39404.5 | | | | |
| CD.EC.APOC3.CUBN | MUC3A | 1.37 | CD.Fibro.LY6H.PAPPA2 | MYLK | 1.53 | CD.Plsma/IgM.IGHM.IGLV2-14 | SEC11C | 1.07 |
| CD.EC.APOC3.CUBN | ENPEP | 1.36 | CD.Fibro.LY6H.PAPPA2 | ADAMTSL3 | 0.74 | CD.Plsma/IgM.IGHM.IGLV2-14 | CCR10 | 0.53 |
| CD.EC.APOC3.CUBN | SLC15A1 | 1.36 | CD.Fibro.LY6H.PAPPA2 | COL6A3 | 1.72 | CD.Plsma/IgM.IGHM.IGLV2-14 | ANKRD28 | 0.85 |
| CD.EC.APOC3.CUBN | SI | 1.35 | CD.Fibro.LY6H.PAPPA2 | CFH | 1.91 | CD.Plsma/IgM.IGHM.JCHAIN | IGHM | 4.52 |
| CD.EC.APOC3.CUBN | RND3 | 1.35 | CD.Fibro.LY6H.PAPPA2 | SMOC2 | 1.54 | CD.Plsma/IgM.IGHM.JCHAIN | JCHAIN | 2.35 |
| CD.EC.APOC3.CUBN | MGAM | 1.33 | CD.Fibro.LY6H.PCSK6 | IGFBP5 | 3.65 | CD.Plsma/IgM.IGHM.JCHAIN | IGKC | 2.35 |
| CD.EC.APOC3.CUBN | LINC01133 | 1.32 | CD.Fibro.LY6H.PCSK6 | APOD | 3.19 | CD.Plsma/IgM.IGHM.JCHAIN | IGKV1-12 | 1.79 |
| CD.EC.APOC3.CUBN | TRIM31 | 1.28 | CD.Fibro.LY6H.PCSK6 | EDNRB | 2.97 | CD.Plsma/IgM.IGHM.JCHAIN | IGKV4-1 | 1.61 |
| CD.EC.APOC3.CUBN | SEMA3B | 1.28 | CD.Fibro.LY6H.PCSK6 | ADH1B | 2.90 | CD.Plsma/IgM.IGHM.JCHAIN | MZB1 | 1.38 |
| CD.EC.APOC3.CUBN | TM4SF5 | 1.28 | CD.Fibro.LY6H.PCSK6 | PDGFRA | 2.73 | CD.Plsma/IgM.IGHM.JCHAIN | TNFRSF17 | 1.24 |
| CD.EC.APOC3.CUBN | ALPI | 1.26 | CD.Fibro.LY6H.PCSK6 | ABI3BP | 2.56 | CD.Plsma/IgM.IGHM.JCHAIN | SSR4 | 1.15 |
| CD.EC.APOC3.CUBN | SLC5A12 | 1.26 | CD.Fibro.LY6H.PCSK6 | F3 | 2.54 | CD.Plsma/IgM.IGHM.JCHAIN | FKBP11 | 1.03 |
| CD.EC.APOC3.CUBN | RP11- | 1.24 | CD.Fibro.LY6H.PCSK6 | LAMA4 | 2.24 | CD.Plsma/IgM.IGHM.JCHAIN | HERPUD1 | 0.97 |
| | 462G2.1 | | | | | | | |
| CD.EC.APOC3.CUBN | ACE2 | 1.23 | CD.Fibro.LY6H.PCSK6 | FN1 | 2.18 | CD.Plsma/IgM.IGHM.JCHAIN | DERL3 | 0.96 |
| CD.EC.APOC3.CUBN | TBX3 | 1.22 | CD.Fibro.LY6H.PCSK6 | SULF1 | 2.17 | CD.Plsma/IgM.IGHM.JCHAIN | SEC11C | 0.96 |
| CD.EC.APOC3.CUBN | NAALADL1 | 1.19 | CD.Fibro.LY6H.PCSK6 | LIMCH1 | 2.00 | CD.Plsma/IgM.IGHM.JCHAIN | XBP1 | 0.96 |
| CD.EC.APOC3.CUBN | HNF4G | 1.18 | CD.Fibro.LY6H.PCSK6 | SMOC2 | 1.76 | CD.Plsma/IgM.IGHM.JCHAIN | PRDX4 | 0.76 |
| CD.EC.APOC3.CUBN | REEP6 | 1.17 | CD.Fibro.LY6H.PCSK6 | PCSK6 | 1.74 | CD.Plsma/IgM.IGHM.JCHAIN | DUSP5 | 0.63 |
| CD.EC.APOC3.CUBN | MEP1A | 1.15 | CD.Fibro.LY6H.PCSK6 | LY6H | 1.73 | CD.Plsma/IgM.IGHM.JCHAIN | UBE2J1 | 0.60 |
| CD.EC.APOC3.CUBN | HHLA2 | 1.15 | CD.Fibro.LY6H.PCSK6 | SOX6 | 1.64 | CD.Plsma/IgM.IGHM.JCHAIN | SSR3 | 0.59 |
| CD.EC.APOC3.CUBN | MYO15B | 1.15 | CD.Fibro.LY6H.PCSK6 | PDGFD | 1.61 | CD.Plsma/IgM.IGHM.JCHAIN | ANKRD28 | 0.59 |
| CD.EC.APOC3.CUBN | SCIN | 1.15 | CD.Fibro.LY6H.PCSK6 | WNT5A | 1.56 | CD.Plsma/IgM.IGHM.JCHAIN | ITM2C | 0.58 |
| CD.EC.APOC3.CUBN | MUC17 | 1.13 | CD.Fibro.LY6H.PCSK6 | PTGIS | 1.44 | CD.Plsma/IgM.IGHM.JCHAIN | PIM2 | 0.58 |
| CD.EC.APOC3.CUBN | MEP1B | 1.12 | CD.Fibro.LY6H.PCSK6 | MAMDC2 | 1.34 | CD.Plsma/IgM.IGHM.JCHAIN | ANKRD37 | 0.58 |
| CD.EC.APOC3.CUBN | NR1H4 | 1.10 | CD.Fibro.LY6H.PCSK6 | TRPA1 | 1.32 | CD.Plsma/IgM.IGHM.JCHAIN | MANF | 0.54 |
| CD.EC.APOC3.CUBN | ESPN | 1.08 | CD.Fibro.LY6H.PCSK6 | FAM150B | 1.24 | CD.Plsma/IgM.IGHM.JCHAIN | VIMP | 0.53 |
| CD.EC.APOC3.CUBN | ABCG2 | 1.06 | CD.Fibro.LY6H.PCSK6 | COL4A5 | 1.15 | CD.Plsma/IgM.IGHM.JCHAIN | CCR10 | 0.50 |
| CD.EC.APOC3.CUBN | SLC51B | 1.04 | CD.Fibro.LY6H.PCSK6 | FIGF | 0.94 | CD.Plsma/IgM.IGHM.JCHAIN | SDF2L1 | 0.48 |
| CD.EC.APOC3.CUBN | XPNPEP2 | 1.03 | CD.Fibro.LY6H.PCSK6 | ADAMTSL3 | 0.88 | CD.Plsma/IgM.IGHM.JCHAIN | CRELD2 | 0.48 |
| CD.EC.APOC3.CUBN | FAM132A | 1.02 | CD.Fibro.LY6H.PCSK6 | GLP2R | 0.84 | CD.Plsma/IgM.IGHM.JCHAIN | RP11- | 0.44 |
| | | | | | | | 290F5.1 | |
| CD.EC.APOC3.CUBN | TMIGD1 | 1.02 | CD.Fibro.LY6H.PCSK6 | F2RL2 | 0.77 | CD.Plsma/IgM.IGHM.JCHAIN | PLPP5 | 0.39 |
| CD.EC.APOC3.CUBN | CDKN2B-AS1 | 1.01 | CD.Fibro.LY6H.PCSK6 | COL6A1 | 2.26 | CD.Plsma/IgM.IGHM.JCHAIN | UAP1 | 0.39 |
| CD.EC.APOC3.CUBN | MS4A8 | 1.00 | CD.Fibro.LY6H.PCSK6 | TAGLN | 2.99 | CD.Plsma/IgM.IGHM.JCHAIN | EAF2 | 0.38 |
| CD.EC.APOC3.CUBN | SLC10A2 | 0.91 | CD.Fibro.LY6H.PCSK6 | SOD3 | 2.44 | CD.Plsma/IgM.IGHM.JCHAIN | TXNDC11 | 0.35 |
| CD.EC.APOC3.CUBN | MOGAT2 | 0.91 | CD.Fibro.LY6H.PCSK6 | COL12A1 | 1.68 | CD.Plsma/IgM.IGHM.JCHAIN | CHPF | 0.33 |
| CD.EC.APOC3.CUBN | SLC2A2 | 0.90 | CD.Fibro.LY6H.PCSK6 | SDC2 | 2.09 | CD.Plsma/IgM.IGHM.JCHAIN | FCRL5 | 0.33 |
| CD.EC.APOC3.CUBN | UGT2B7 | 0.88 | CD.Fibro.LY6H.PCSK6 | GPC6 | 1.19 | CD.Plsma/IgM.IGHM.JCHAIN | GNG7 | 0.28 |
| CD.EC.APOC3.CUBN | CPO | 0.86 | CD.Fibro.NPY.HSD17B2 | TAGLN | 2.99 | CD.Plsma/IgM.IGHM.JCHAIN | SDC1 | 0.37 |
| CD.EC.APOC3.CUBN | CIDEC | 0.82 | CD.Fibro.NPY.HSD17B2 | EDNRB | 2.70 | CD.Plsma/IgM.IGHM.JCHAIN | JUN | 0.95 |
| CD.EC.APOC3.CUBN | SUSD2 | 0.79 | CD.Fibro.NPY.HSD17B2 | APOD | 2.45 | CD.Plsma/IgM.IGHM.JCHAIN | DNAJB9 | 0.52 |
| CD.EC.APOC3.CUBN | ENPP7 | 0.66 | CD.Fibro.NPY.HSD17B2 | FRZB | 2.10 | CD.Plsma/IgM.IGHM.JCHAIN | POU2AF1 | 0.26 |
| CD.EC.APOC3.CUBN | DEFA5 | 0.53 | CD.Fibro.NPY.HSD17B2 | BMP4 | 2.02 | CD.Plsma/IgM.IGHM.JCHAIN | FAM46C | 0.33 |
| CD.EC.APOC3.CUBN | DEFA6 | 0.52 | CD.Fibro.NPY.HSD17B2 | ACTG2 | 1.90 | CD.Prolif/EC.UBE2C.CCNB | MT1G | 2.42 |
| CD.EC.APOC3.CUBN | SLC6A8 | 0.88 | CD.Fibro.NPY.HSD17B2 | NPY | 1.72 | CD.Prolif/EC.UBE2C.CCNB2 | OLFM4 | 2.39 |
| CD.EC.APOC3.CUBN | TREH | 0.69 | CD.Fibro.NPY.HSD17B2 | TMEM119 | 1.64 | CD.Prolif/EC.UBE2C.CCNB2 | TSPAN8 | 2.24 |
| CD.EC.APOC3.CUBN | SLC13A1 | 0.66 | CD.Fibro.NPY.HSD17B2 | ENHO | 1.40 | CD.Prolif/EC.UBE2C.CCNB2 | REG1A | 2.24 |
| CD.EC.APOC3.CUBN | MFSD2A | 1.08 | CD.Fibro.NPY.HSD17B2 | INSC | 1.07 | CD.Prolif/EC.UBE2C.CCNB2 | MT1E | 2.21 |
| CD.EC.APOC3.CUBN | LIPH | 1.21 | CD.Fibro.OGN.C1QTNF3 | OGN | 2.13 | CD.Prolif/EC.UBE2C.CCNB2 | EPCAM | 2.10 |
| CD.EC.APOC3.CUBN | MALL | 1.15 | CD.Fibro.OGN.C1QTNF3 | RP11- | 0.82 | CD.Prolif/EC.UBE2C.CCNB2 | MT1H | 2.02 |
| | | | | 135D11.2 | | | | |
| CD.EC.APOC3.CUBN | ERICH4 | 0.75 | CD.Fibro.OGN.C1QTNF3 | CCDC80 | 2.08 | CD.Prolif/EC.UBE2C.CCNB2 | LGALS4 | 2.02 |
| CD.EC.APOC3.CUBN | UGT2A3 | 1.06 | CD.Fibro.OGN.C1QTNF3 | CTB-92J24.3 | 0.69 | CD.Prolif/EC.UBE2C.CCNB2 | CCL25 | 1.92 |
| CD.EC.APOC3.CUBN | ABCB1 | 1.29 | CD.Fibro.OGN.C1QTNF3 | C7 | 2.10 | CD.Prolif/EC.UBE2C.CCNB2 | KRT8 | 1.90 |
| CD.EC.APOC3.CUBN | CES2 | 1.42 | CD.Fibro.PCDH18.APOE | CXCL6 | 2.20 | CD.Prolif/EC.UBE2C.CCNB2 | KRT18 | 1.90 |
| CD.EC.APOC3.CUBN | ABCC2 | 0.61 | CD.Fibro.PCDH18.APOE | PCDH18 | 1.89 | CD.Prolif/EC.UBE2C.CCNB2 | MT2A | 1.83 |
| CD.EC.APOC3.CUBN | GPD1 | 0.85 | CD.Fibro.PCDH18.APOE | DCN | 2.90 | CD.Prolif/EC.UBE2C.CCNB2 | PIGR | 1.80 |
| CD.EC.APOC3.CUBN | SULT1B1 | 0.96 | CD.Fibro.PCSK6.FIGF | IGFBP5 | 3.51 | CD.Prolif/EC.UBE2C.CCNB2 | AGR2 | 1.75 |
| CD.EC.APOC3.CUBN | MTRNR2L12 | 1.61 | CD.Fibro.PCSK6.FIGF | APOD | 3.45 | CD.Prolif/EC.UBE2C.CCNB2 | GPX2 | 1.60 |
| CD.EC.APOC3.CUBN | C6orf222 | 0.94 | CD.Fibro.PCSK6.FIGF | ACTG2 | 1.76 | CD.Prolif/EC.UBE2C.CCNB2 | MGST1 | 1.59 |
| CD.EC.APOC3.CUBN | GNA11 | 1.10 | CD.Fibro.PCSK6.FIGF | FIGF | 1.65 | CD.Prolif/EC.UBE2C.CCNB2 | SPINK1 | 1.54 |
| CD.EC.APOC3.CUBN | EPS8L3 | 1.13 | CD.Fibro.PCSK6.FIGF | PCSK6 | 1.52 | CD.Prolif/EC.UBE2C.CCNB2 | KRT19 | 1.54 |
| CD.EC.CFTR.OLFM4 | APOA1 | 0.47 | CD.Fibro.PCSK6.FIGF | LY6H | 1.47 | CD.Prolif/EC.UBE2C.CCNB2 | MT1F | 1.48 |
| CD.EC.CFTR.OLFM4 | APOA4 | 0.59 | CD.Fibro.PCSK6.FIGF | PTGIS | 1.12 | CD.Prolif/EC.UBE2C.CCNB2 | MT1M | 1.47 |
| CD.EC.FABP1.ADIRF | FABP1 | 3.36 | CD.Fibro.PCSK6.FIGF | EDNRB | 2.32 | CD.Prolif/EC.UBE2C.CCNB2 | C15orf48 | 1.45 |
| CD.EC.FABP1.ADIRF | PHGR1 | 3.17 | CD.Fibro.PCSK6.FIGF | TAGLN | 3.67 | CD.Prolif/EC.UBE2C.CCNB2 | DMBT1 | 1.43 |
| CD.EC.FABP1.ADIRF | ALDOB | 2.60 | CD.Fibro.PCSK6.FIGF | PDGFRA | 1.99 | CD.Prolif/EC.UBE2C.CCNB2 | PLA2G2A | 1.41 |
| CD.EC.FABP1.ADIRF | PRAP1 | 2.59 | CD.Fibro.PCSK6.FIGF | ADAMTSL3 | 0.82 | CD.Prolif/EC.UBE2C.CCNB2 | CLDN7 | 1.41 |
| CD.EC.FABP1.ADIRF | RBP2 | 2.59 | CD.Fibro.PHGDH.STMN2 | LUM | 2.85 | CD.Prolif/EC.UBE2C.CCNB2 | ANXA2 | 1.39 |
| CD.EC.FABP1.ADIRF | KRT8 | 2.49 | CD.Fibro.PHGDH.STMN2 | ACTA2 | 2.70 | CD.Prolif/EC.UBE2C.CCNB2 | LGALS3 | 1.38 |
| CD.EC.FABP1.ADIRF | GSTA1 | 2.47 | CD.Fibro.PHGDH.STMN2 | TAGLN | 2.48 | CD.Prolif/EC.UBE2C.CCNB2 | AGR3 | 1.35 |
| CD.EC.FABP1.ADIRF | KRT19 | 2.46 | CD.Fibro.PHGDH.STMN2 | DCN | 2.29 | CD.Prolif/EC.UBE2C.CCNB2 | AKR1C3 | 1.32 |
| CD.EC.FABP1.ADIRF | ADIRF | 2.40 | CD.Fibro.PHGDH.STMN2 | FBLN1 | 2.14 | CD.Prolif/EC.UBE2C.CCNB2 | KLF5 | 1.32 |
| CD.EC.FABP1.ADIRF | LGALS4 | 2.22 | CD.Fibro.PHGDH.STMN2 | MMP2 | 2.09 | CD.Prolif/EC.UBE2C.CCNB2 | MLEC | 1.29 |
| CD.EC.FABP1.ADIRF | C19orf33 | 2.08 | CD.Fibro.PHGDH.STMN2 | MYLK | 1.87 | CD.Prolif/EC.UBE2C.CCNB2 | UGT2B17 | 1.27 |
| CD.EC.FABP1.ADIRF | ANPEP | 1.98 | CD.Fibro.PHGDH.STMN2 | STMN2 | 1.64 | CD.Prolif/EC.UBE2C.CCNB2 | SLC12A2 | 1.25 |
| CD.EC.FABP6.PLCG2 | FABP6 | 3.71 | CD.Fibro.PHGDH.STMN2 | TPM2 | 2.02 | CD.Prolif/EC.UBE2C.CCNB2 | UBE2C | 1.23 |
| CD.EC.FABP6.PLCG2 | ANPEP | 2.84 | CD.Fibro.PHGDH.STMN2 | C15 | 2.48 | CD.Prolif/EC.UBE2C.CCNB2 | MDK | 1.19 |
| CD.EC.FABP6.PLCG2 | FABP1 | 2.73 | CD.Fibro.RAMP1.IGHG1 | IGFBP5 | 3.22 | CD.Prolif/EC.UBE2C.CCNB2 | COX5A | 1.18 |
| CD.EC.FABP6.PLCG2 | CDHR5 | 2.51 | CD.Fibro.RAMP1.IGHG1 | CXCL6 | 2.79 | CD.Prolif/EC.UBE2C.CCNB2 | CLDN3 | 1.18 |
| CD.EC.FABP6.PLCG2 | ALDOB | 2.61 | CD.Fibro.RAMP1.IGHG1 | SFRP1 | 2.62 | CD.Prolif/EC.UBE2C.CCNB2 | CLDN4 | 1.17 |
| CD.EC.FABP6.PLCG2 | PRAP1 | 2.88 | CD.Fibro.RAMP1.IGHG1 | TFPI2 | 1.90 | CD.Prolif/EC.UBE2C.CCNB2 | CD9 | 1.16 |
| CD.EC.FABP6.SLC26A3 | FABP6 | 3.81 | CD.Fibro.RAMP1.IGHG1 | PAPPA | 1.45 | CD.Prolif/EC.UBE2C.CCNB2 | PHGR1 | 1.15 |
| CD.EC.FABP6.SLC26A3 | PRAP1 | 3.19 | CD.Fibro.RAMP1.IGHG1 | CCL11 | 4.25 | CD.Prolif/EC.UBE2C.CCNB2 | PTTG1 | 1.14 |
| CD.EC.FABP6.SLC26A3 | FABP1 | 3.06 | CD.Fibro.RERGL.NTRK2 | ACTA2 | 3.92 | CD.Prolif/EC.UBE2C.CCNB2 | IDH2 | 1.13 |
| CD.EC.FABP6.SLC26A3 | PHGR1 | 2.97 | CD.Fibro.RERGL.NTRK2 | TAGLN | 3.84 | CD.Prolif/EC.UBE2C.CCNB2 | MT1X | 1.12 |
| CD.EC.FABP6.SLC26A3 | FABP2 | 2.87 | CD.Fibro.RERGL.NTRK2 | MYL9 | 3.65 | CD.Prolif/EC.UBE2C.CCNB2 | REG1B | 1.12 |
| CD.EC.FABP6.SLC26A3 | ALDOB | 2.86 | CD.Fibro.RERGL.NTRK2 | ADIRF | 3.43 | CD.Prolif/EC.UBE2C.CCNB2 | IFI27 | 1.11 |
| CD.EC.FABP6.SLC26A3 | ADIRF | 2.60 | CD.Fibro.RERGL.NTRK2 | TPM2 | 3.16 | CD.Prolif/EC.UBE2C.CCNB2 | ATP5G1 | 1.09 |
| CD.EC.FABP6.SLC26A3 | ANPEP | 2.58 | CD.Fibro.RERGL.NTRK2 | SPARCL1 | 3.04 | CD.Prolif/EC.UBE2C.CCNB2 | TOP2A | 1.07 |
| CD.EC.FABP6.SLC26A3 | RBP2 | 2.57 | CD.Fibro.RERGL.NTRK2 | MYH11 | 2.81 | CD.Prolif/EC.UBE2C.CCNB2 | TXN | 1.05 |
| CD.EC.FABP6.SLC26A3 | SLC26A3 | 2.43 | CD.Fibro.RERGL.NTRK2 | RGS5 | 2.74 | CD.Prolif/EC.UBE2C.CCNB2 | CES2 | 1.05 |
| CD.EC.FABP6.SLC26A3 | APOB | 2.40 | CD.Fibro.RERGL.NTRK2 | RERGL | 2.73 | CD.Prolif/EC.UBE2C.CCNB2 | HMGA1 | 1.03 |
| CD.EC.FABP6.SLC26A3 | CDHR5 | 2.30 | CD.Fibro.RERGL.NTRK2 | CALD1 | 2.72 | CD.Prolif/EC.UBE2C.CCNB2 | ELF3 | 1.02 |
| CD.EC.FABP6.SLC26A3 | GUCAZA | 2.26 | CD.Fibro.RERGL.NTRK2 | SOD3 | 2.69 | CD.Prolif/EC.UBE2C.CCNB2 | ATP1B1 | 1.02 |
| CD.EC.FABP6.SLC26A3 | MTTP | 2.22 | CD.Fibro.RERGL.NTRK2 | IGFBP7 | 2.56 | CD.Prolif/EC.UBE2C.CCNB2 | CYC1 | 1.00 |
| CD.EC.FABP6.SLC26A3 | DPEP1 | 2.19 | CD.Fibro.RERGL.NTRK2 | PLN | 2.53 | CD.Prolif/EC.UBE2C.CCNB2 | ADH1C | 0.98 |
| CD.EC.FABP6.SLC26A3 | APOA4 | 2.15 | CD.Fibro.RERGL.NTRK2 | FRZB | 2.47 | CD.Prolif/EC.UBE2C.CCNB2 | CENPF | 0.98 |
| CD.EC.FABP6.SLC26A3 | APOA1 | 2.02 | CD.Fibro.RERGL.NTRK2 | CAV1 | 2.45 | CD.Prolif/EC.UBE2C.CCNB2 | IGFBP2 | 0.98 |
| CD.EC.FABP6.SLC26A3 | AMN | 2.00 | CD.Fibro.RERGL.NTRK2 | MFGE8 | 2.25 | CD.Prolif/EC.UBE2C.CCNB2 | CDX1 | 0.98 |
| CD.EC.FABP6.SLC26A3 | ACE | 2.00 | CD.Fibro.RERGL.NTRK2 | CSRP2 | 2.21 | CD.Prolif/EC.UBE2C.CCNB2 | S100A14 | 0.97 |
| CD.EC.FABP6.SLC26A3 | DGAT1 | 1.98 | CD.Fibro.RERGL.NTRK2 | PRKCDBP | 2.19 | CD.Prolif/EC.UBE2C.CCNB2 | ID1 | 0.96 |
| CD.EC.FABP6.SLC26A3 | SI | 1.98 | CD.Fibro.RERGL.NTRK2 | IGFBP5 | 2.17 | CD.Prolif/EC.UBE2C.CCNB2 | TUBA1C | 0.95 |
| CD.EC.FABP6.SLC26A3 | C19orf33 | 1.98 | CD.Fibro.RERGL.NTRK2 | NDUFA4L2 | 2.00 | CD.Prolif/EC.UBE2C.CCNB2 | CCNB1 | 0.95 |
| CD.EC.FABP6.SLC26A3 | PLS1 | 1.94 | CD.Fibro.RERGL.NTRK2 | MAP3K7CL | 1.84 | CD.Prolif/EC.UBE2C.CCNB2 | MKI67 | 0.95 |
| CD.EC.FABP6.SLC26A3 | PCK1 | 1.94 | CD.Fibro.RERGL.NTRK2 | SERPINI1 | 1.82 | CD.Prolif/EC.UBE2C.CCNB2 | EDN1 | 0.94 |
| CD.EC.FABP6.SLC26A3 | CYP3A5 | 1.86 | CD.Fibro.RERGL.NTRK2 | PTRF | 1.82 | CD.Prolif/EC.UBE2C.CCNB2 | CENPW | 0.94 |
| CD.EC.FABP6.SLC26A3 | APOC3 | 1.76 | CD.Fibro.RERGL.NTRK2 | GPX3 | 1.81 | CD.Prolif/EC.UBE2C.CCNB2 | FAM3B | 0.94 |
| CD.EC.FABP6.SLC26A3 | CUBN | 1.70 | CD.Fibro.RERGL.NTRK2 | TINAGL1 | 1.80 | CD.Prolif/EC.UBE2C.CCNB2 | MGST2 | 0.93 |
| CD.EC.FABP6.SLC26A3 | MYO1A | 1.69 | CD.Fibro.RERGL.NTRK2 | PPP1R14A | 1.77 | CD.Prolif/EC.UBE2C.CCNB2 | SUCLG1 | 0.92 |
| CD.EC.FABP6.SLC26A3 | MME | 1.60 | CD.Fibro.RERGL.NTRK2 | BCAM | 1.75 | CD.Prolif/EC.UBE2C.CCNB2 | TMEM54 | 0.92 |
| CD.EC.FABP6.SLC26A3 | C11orf86 | 1.37 | CD.Fibro.RERGL.NTRK2 | TIMP3 | 1.75 | CD.Prolif/EC.UBE2C.CCNB2 | PPP1R1B | 0.92 |
| CD.EC.FABP6.SLC26A3 | SLC6A19 | 1.36 | CD.Fibro.RERGL.NTRK2 | MYLK | 1.74 | CD.Prolif/EC.UBE2C.CCNB2 | SMIM22 | 0.92 |
| CD.EC.FABP6.SLC26A3 | FLJ22763 | 1.35 | CD.Fibro.RERGL.NTRK2 | SNCG | 1.73 | CD.Prolif/EC.UBE2C.CCNB2 | SPINT2 | 0.92 |
| CD.EC.FABP6.SLC26A3 | CDHR2 | 1.33 | CD.Fibro.RERGL.NTRK2 | ACTG2 | 1.68 | CD.Prolif/EC.UBE2C.CCNB2 | STMN1 | 0.92 |
| CD.EC.FABP6.SLC26A3 | GDA | 1.32 | CD.Fibro.RERGL.NTRK2 | RCAN2 | 1.65 | CD.Prolif/EC.UBE2C.CCNB2 | ACAA2 | 0.90 |
| CD.EC.FABP6.SLC26A3 | SULT1A2 | 1.30 | CD.Fibro.RERGL.NTRK2 | AC013461.1 | 1.63 | CD.Prolif/EC.UBE2C.CCNB2 | MPC2 | 0.89 |
| CD.EC.FABP6.SLC26A3 | MGAM | 1.27 | CD.Fibro.RERGL.NTRK2 | SORBS2 | 1.62 | CD.Prolif/EC.UBE2C.CCNB2 | KIAA0101 | 0.88 |
| CD.EC.FABP6.SLC26A3 | ACE2 | 1.26 | CD.Fibro.RERGL.NTRK2 | PLAC9 | 1.56 | CD.Prolif/EC.UBE2C.CCNB2 | CKS2 | 0.88 |
| CD.EC.FABP6.SLC26A3 | SLC5A12 | 1.12 | CD.Fibro.RERGL.NTRK2 | CRIP2 | 1.54 | CD.Prolif/EC.UBE2C.CCNB2 | KRTCAP3 | 0.87 |
| CD.EC.FABP6.SLC26A3 | KRT20 | 1.86 | CD.Fibro.RERGL.NTRK2 | C11orf96 | 1.53 | CD.Prolif/EC.UBE2C.CCNB2 | ECHS1 | 0.84 |
| CD.EC.FABP6.SLC26A3 | DST | 1.98 | CD.Fibro.RERGL.NTRK2 | CAV2 | 1.52 | CD.Prolif/EC.UBE2C.CCNB2 | CYSTM1 | 0.84 |
| CD.EC.FABP6.SLC26A3 | ANGPTL4 | 1.54 | CD.Fibro.RERGL.NTRK2 | LMOD1 | 1.42 | CD.Prolif/EC.UBE2C.CCNB2 | PERP | 0.84 |
| CD.EC.FABP6.SLC26A3 | SLC5A1 | 1.46 | CD.Fibro.RERGL.NTRK2 | COX412 | 1.40 | CD.Prolif/EC.UBE2C.CCNB2 | FABP2 | 0.83 |
| CD.EC.GNAT3.TRPM5 | SH2D6 | 2.92 | CD.Fibro.RERGL.NTRK2 | CPE | 1.39 | CD.Prolif/EC.UBE2C.CCNB2 | S100A16 | 0.83 |
| CD.EC.GNAT3.TRPM5 | TRPM5 | 2.56 | CD.Fibro.RERGL.NTRK2 | NOTCH3 | 1.39 | CD.Prolif/EC.UBE2C.CCNB2 | VDAC1 | 0.83 |
| CD.EC.GNAT3.TRPM5 | BMX | 2.40 | CD.Fibro.RERGL.NTRK2 | MAP1B | 1.34 | CD.Prolif/EC.UBE2C.CCNB2 | SFN | 0.82 |
| CD.EC.GNAT3.TRPM5 | RP11- | 1.81 | CD.Fibro.RERGL.NTRK2 | CRYAB | 1.31 | CD.Prolif/EC.UBE2C.CCNB2 | ALDH2 | 0.81 |
| | 295M3.4 | | | | | | | |
| CD.EC.GNAT3.TRPM5 | GNAT3 | 1.81 | CD.Fibro.RERGL.NTRK2 | GUCY1A3 | 1.31 | CD.Prolif/EC.UBE2C.CCNB2 | SERPINB6 | 0.81 |
| CD.EC.GNAT3.TRPM5 | SH2D7 | 1.46 | CD.Fibro.RERGL.NTRK2 | MCAM | 1.31 | CD.Prolif/EC.UBE2C.CCNB2 | ALDH1A1 | 0.81 |
| CD.EC.GNAT3.TRPM5 | CCDC129 | 1.26 | CD.Fibro.RERGL.NTRK2 | BGN | 1.29 | CD.Prolif/EC.UBE2C.CCNB2 | PHLDA2 | 0.81 |
| CD.EC.GNAT3.TRPM5 | GNG13 | 1.42 | CD.Fibro.RERGL.NTRK2 | FILIP1 | 1.28 | CD.Prolif/EC.UBE2C.CCNB2 | TMEM106C | 0.81 |
| CD.EC.GSTA2.AADAC | GSTA2 | 1.91 | CD.Fibro.RERGL.NTRK2 | PDGFRB | 1.27 | CD.Prolif/EC.UBE2C.CCNB2 | LIMA1 | 0.80 |
| CD.EC.GSTA2.AADAC | AKR1B10 | 1.35 | CD.Fibro.RERGL.NTRK2 | CNN1 | 1.26 | CD.Prolif/EC.UBE2C.CCNB2 | RAB25 | 0.79 |
| CD.EC.GSTA2.AADAC | SLC51B | 1.29 | CD.Fibro.RERGL.NTRK2 | TBX2-AS1 | 1.24 | CD.Prolif/EC.UBE2C.CCNB2 | ETHE1 | 0.79 |
| CD.EC.GSTA2.AADAC | AADAC | 1.14 | CD.Fibro.RERGL.NTRK2 | MSRB3 | 1.10 | CD.Prolif/EC.UBE2C.CCNB2 | HES1 | 0.79 |
| CD.EC.GSTA2.AADAC | SLC7A9 | 1.13 | CD.Fibro.RERGL.NTRK2 | NTRK2 | 1.07 | CD.Prolif/EC.UBE2C.CCNB2 | FABP1 | 0.78 |
| CD.EC.GSTA2.AADAC | FLJ22763 | 1.02 | CD.Fibro.RERGL.NTRK2 | CCDC3 | 1.04 | CD.Prolif/EC.UBE2C.CCNB2 | TPM1 | 0.78 |
| CD.EC.GSTA2.AADAC | TMPRSS15 | 0.95 | CD.Fibro.RERGL.NTRK2 | CAP2 | 0.96 | CD.Prolif/EC.UBE2C.CCNB2 | PDLIM1 | 0.78 |
| CD.EC.GSTA2.AADAC | ACE2 | 0.89 | CD.Fibro.RERGL.NTRK2 | NRGN | 0.96 | CD.Prolif/EC.UBE2C.CCNB2 | CKS1B | 0.77 |
| CD.EC.GSTA2.AADAC | BTNL3 | 0.85 | CD.Fibro.RERGL.NTRK2 | EFHD1 | 0.91 | CD.Prolif/EC.UBE2C.CCNB2 | CISD3 | 0.77 |
| CD.EC.GSTA2.AADAC | C1orf210 | 0.64 | CD.Fibro.RERGL.NTRK2 | TBX2 | 0.89 | CD.Prolif/EC.UBE2C.CCNB2 | SELENBP1 | 0.77 |
| CD.EC.GSTA2.AADAC | COL17A1 | 0.61 | CD.Fibro.RERGL.NTRK2 | EDNRA | 0.87 | CD.Prolif/EC.UBE2C.CCNB2 | ANXA4 | 0.76 |
| CD.EC.GSTA2.AADAC | AC004754.3 | 0.56 | CD.Fibro.RERGL.NTRK2 | SGCA | 0.86 | CD.Prolif/EC.UBE2C.CCNB2 | BIRC5 | 0.76 |
| CD.EC.GSTA2.AADAC | TMEM82 | 0.53 | CD.Fibro.RERGL.NTRK2 | NPY1R | 0.85 | CD.Prolif/EC.UBE2C.CCNB2 | CDC20 | 0.76 |
| CD.EC.GSTA2.AADAC | SLC26A3 | 0.96 | CD.Fibro.RERGL.NTRK2 | C2orf40 | 0.85 | CD.Prolif/EC.UBE2C.CCNB2 | NUSAP1 | 0.76 |
| CD.EC.GSTA2.AADAC | PDZK1 | 0.55 | CD.Fibro.RERGL.NTRK2 | ITGA7 | 0.79 | CD.Prolif/EC.UBE2C.CCNB2 | HNRNPAB | 0.75 |
| CD.EC.GSTA2.AADAC | ERICH4 | 0.53 | CD.Fibro.RERGL.NTRK2 | OR51E1 | 0.75 | CD.Prolif/EC.UBE2C.CCNB2 | MAOA | 0.75 |
| CD.EC.GSTA2.AADAC | SLC51A | 0.72 | CD.Fibro.RERGL.NTRK2 | FAM162B | 0.74 | CD.Prolif/EC.UBE2C.CCNB2 | PCBD1 | 0.74 |
| CD.EC.GSTA2.AADAC | ARHGDIG | 0.77 | CD.Fibro.RERGL.NTRK2 | GPR20 | 0.70 | CD.Prolif/EC.UBE2C.CCNB2 | CPS1 | 0.74 |
| CD.EC.GSTA2.AADAC | RP11- | 0.59 | CD.Fibro.RERGL.NTRK2 | ENTPD3 | 0.57 | CD.Prolif/EC.UBE2C.CCNB2 | CCNB2 | 0.74 |
| | 680F8.1 | | | | | | | |
| CD.EC.GSTA2.CES3 | GSTA2 | 1.51 | CD.Fibro.RERGL.NTRK2 | LDB3 | 0.51 | CD.Prolif/EC.UBE2C.CCNB2 | CDC42EP5 | 0.74 |
| CD.EC.GSTA2.TMPRSS15 | GSTA2 | 1.71 | CD.Fibro.RERGL.NTRK2 | CDH6 | 0.83 | CD.Prolif/EC.UBE2C.CCNB2 | CDX2 | 0.74 |
| CD.EC.GSTA2.TMPRSS15 | SLC51B | 1.30 | CD.Fibro.RERGL.NTRK2 | GJA4 | 0.86 | CD.Prolif/EC.UBE2C.CCNB2 | PLP2 | 0.74 |
| CD.EC.GSTA2.TMPRSS15 | AKR1B10 | 1.19 | CD.Fibro.RERGL.NTRK2 | LGI4 | 0.79 | CD.Prolif/EC.UBE2C.CCNB2 | CASP1 | 0.73 |
| CD.EC.GSTA2.TMPRSS15 | AADAC | 1.04 | CD.Fibro.RERGL.NTRK2 | HRC | 0.71 | CD.Prolif/EC.UBE2C.CCNB2 | UQCRC1 | 0.73 |
| CD.EC.GSTA2.TMPRSS15 | TMPRSS15 | 0.94 | CD.Fibro.RERGL.NTRK2 | COX7A1 | 1.47 | CD.Prolif/EC.UBE2C.CCNB2 | GOLM1 | 0.72 |
| CD.EC.GSTA2.TMPRSS15 | SULT1E1 | 0.93 | CD.Fibro.RERGL.NTRK2 | FOXS1 | 0.74 | CD.Prolif/EC.UBE2C.CCNB2 | DSP | 0.71 |
| CD.EC.GSTA2.TMPRSS15 | APOBEC1 | 0.57 | CD.Fibro.RERGL.NTRK2 | C1QTNF1 | 0.91 | CD.Prolif/EC.UBE2C.CCNB2 | HMGB3 | 0.71 |
| CD.EC.GSTA2.TMPRSS15 | C17orf78 | 0.83 | CD.Fibro.RERGL.NTRK2 | NET1 | 2.00 | CD.Prolif/EC.UBE2C.CCNB2 | DHRS11 | 0.71 |
| CD.EC.GSTA2.TMPRSS15 | METTL7B | 0.79 | CD.Fibro.RERGL.NTRK2 | NR2F2-AS1 | 0.78 | CD.Prolif/EC.UBE2C.CCNB2 | CDKN3 | 0.71 |
| CD.EC.GSTA2.TMPRSS15 | SLC7A9 | 0.59 | CD.Fibro.RERGL.NTRK2 | C1QTNF2 | 0.75 | CD.Prolif/EC.UBE2C.CCNB2 | COA3 | 0.71 |
| CD.EC.HSPA1B.PSMA2 | CCL15 | 1.28 | CD.Fibro.RPS4Y1.DEFA5 | CCL11 | 3.02 | CD.Prolif/EC.UBE2C.CCNB2 | ALDOB | 0.70 |
| CD.EC.HSPA1B.PSMA2 | KCNJ13 | 1.04 | CD.Fibro.RPS4Y1.DEFA5 | LUM | 3.01 | CD.Prolif/EC.UBE2C.CCNB2 | RP11- | 0.70 |
| | | | | | | | 357H14.17 | |
| CD.EC.LECT1.FAM26F | CCL15 | 1.55 | CD.Fibro.RPS4Y1.DEFA5 | C1S | 2.55 | CD.Prolif/EC.UBE2C.CCNB2 | CDK1 | 0.70 |
| CD.EC.LECT1.FAM26F | SERPINB5 | 0.77 | CD.Fibro.RPS4Y1.DEFA5 | DCN | 2.36 | CD.Prolif/EC.UBE2C.CCNB2 | GMDS | 0.70 |
| CD.EC.LECT1.FAM26F | DSC2 | 0.75 | CD.Fibro.RPS4Y1.DEFA5 | FBLN1 | 2.33 | CD.Prolif/EC.UBE2C.CCNB2 | PLPP2 | 0.70 |
| CD.EC.LECT1.FAM26F | CBLC | 0.65 | CD.Fibro.RPS4Y1.DEFA5 | MMP2 | 2.19 | CD.Prolif/EC.UBE2C.CCNB2 | CCDC34 | 0.70 |
| CD.EC.LECT1.FAM26F | SDR16C5 | 0.62 | CD.Fibro.RPS4Y1.DEFA5 | TCF21 | 1.80 | CD.Prolif/EC.UBE2C.CCNB2 | EBP | 0.70 |
| CD.EC.LECT1.FAM26F | GABRP | 0.54 | CD.Fibro.RPS4Y1.DEFA5 | CP | 1.64 | CD.Prolif/EC.UBE2C.CCNB2 | CD24 | 0.69 |
| CD.EC.LECT1.FAM26F | RP11- | 0.53 | CD.Fibro.RPS4Y1.DEFA5 | MFAP4 | 1.96 | CD.Prolif/EC.UBE2C.CCNB2 | PCNA | 0.69 |
| | 519G16.5 | | | | | | | |
| CD.EC.LECT1.FAM26F | LECT1 | 0.51 | CD.Fibro.RPS4Y1.DEFA5 | CXCL6 | 1.63 | CD.Prolif/EC.UBE2C.CCNB2 | FXYD3 | 0.69 |
| CD.EC.LECT1.FAM26F | CTSE | 0.39 | CD.Fibro.STMN2.FABP5 | CXCL6 | 2.18 | CD.Prolif/EC.UBE2C.CCNB2 | GPR160 | 0.69 |
| CD.EC.LECT1.FAM26F | GJB3 | 0.34 | CD.Fibro.STMN2.FABP5 | CP | 2.17 | CD.Prolif/EC.UBE2C.CCNB2 | HDGF | 0.69 |
| CD.EC.LECT1.FAM26F | AP1M2 | 0.69 | CD.Fibro.STMN2.FABP5 | STMN2 | 1.75 | CD.Prolif/EC.UBE2C.CCNB2 | PHB | 0.69 |
| CD.EC.LECT1.FAM26F | FERMT1 | 0.76 | CD.Fibro.STMN2.FABP5 | TCF21 | 2.16 | CD.Prolif/EC.UBE2C.CCNB2 | PRSS3 | 0.69 |
| CD.EC.LECT1.FAM26F | CKMT1A | 0.51 | CD.Fibro.TFPI2.CCL13 | CFD | 4.16 | CD.Prolif/EC.UBE2C.CCNB2 | TIMM13 | 0.68 |
| CD.EC.LECT1.FAM26F | TMEM45B | 0.72 | CD.Fibro.TFPI2.CCL13 | CCL13 | 3.81 | CD.Prolif/EC.UBE2C.CCNB2 | LGALS3BP | 0.68 |
| CD.EC.LECT1.FAM26F | AKR1C3 | 1.64 | CD.Fibro.TFPI2.CCL13 | PTGDS | 3.63 | CD.Prolif/EC.UBE2C.CCNB2 | IMPDH2 | 0.67 |
| CD.EC.LECT1.FAM26F | STAP2 | 0.98 | CD.Fibro.TFPI2.CCL13 | CCL11 | 3.43 | CD.Prolif/EC.UBE2C.CCNB2 | GGCT | 0.67 |
| CD.EC.LECT1.FAM26F | OTC | 0.67 | CD.Fibro.TFPI2.CCL13 | LUM | 2.98 | CD.Prolif/EC.UBE2C.CCNB2 | OAT | 0.67 |
| CD.EC.LECT1.FAM26F | PPP1R1B | 1.15 | CD.Fibro.TFPI2.CCL13 | DCN | 2.90 | CD.Prolif/EC.UBE2C.CCNB2 | DSG2 | 0.66 |
| CD.EC.LECT1.FAM26F | PKP2 | 0.72 | CD.Fibro.TFPI2.CCL13 | RARRES2 | 2.89 | CD.Prolif/EC.UBE2C.CCNB2 | SOX4 | 0.66 |
| CD.EC.LECT1.FAM26F | ADH1C | 1.13 | CD.Fibro.TFPI2.CCL13 | APOE | 2.85 | CD.Prolif/EC.UBE2C.CCNB2 | MARCKS | 0.65 |
| CD.EC.LECT1.FAM26F | NOXO1 | 0.47 | CD.Fibro.TFPI2.CCL13 | A2M | 2.60 | CD.Prolif/EC.UBE2C.CCNB2 | CLDN15 | 0.65 |
| CD.EC.LECT1.FAM26F | IGFBP2 | 1.12 | CD.Fibro.TFPI2.CCL13 | ADAMDEC1 | 2.52 | CD.Prolif/EC.UBE2C.CCNB2 | FERMT1 | 0.64 |
| CD.EC.MIF.MTRNR2L8 | SPINK1 | 2.40 | CD.Fibro.TFPI2.CCL13 | FBLN1 | 2.48 | CD.Prolif/EC.UBE2C.CCNB2 | EMP2 | 0.64 |
| CD.EC.MIF.MTRNR2L8 | APOA1 | 0.70 | CD.Fibro.TFPI2.CCL13 | APOD | 2.40 | CD.Prolif/EC.UBE2C.CCNB2 | HADH | 0.64 |
| CD.EC.MIF.MTRNR2L8 | MTRNR2L8 | 3.00 | CD.Fibro.TFPI2.CCL13 | PLPP1 | 2.38 | CD.Prolif/EC.UBE2C.CCNB2 | RRM2 | 0.64 |
| CD.EC.MIF.MTRNR2L8 | MT1G | 2.21 | CD.Fibro.TFPI2.CCL13 | MFAP4 | 2.36 | CD.Prolif/EC.UBE2C.CCNB2 | FAM3D | 0.64 |
| CD.EC.MT-CO2.MT-CO3 | REG1A | 2.49 | CD.Fibro.TFPI2.CCL13 | CCL2 | 2.36 | CD.Prolif/EC.UBE2C.CCNB2 | ETS2 | 0.64 |
| CD.EC.MT-CO2.MT-CO3 | REG1B | 2.24 | CD.Fibro.TFPI2.CCL13 | IGFBP5 | 2.36 | CD.Prolif/EC.UBE2C.CCNB2 | HEBP2 | 0.63 |
| CD.EC.MT-CO2.MT-CO3 | MT1G | 2.08 | CD.Fibro.TFPI2.CCL13 | C1S | 2.33 | CD.Prolif/EC.UBE2C.CCNB2 | RAMP1 | 0.63 |
| CD.EC.MT-CO2.MT-CO3 | OLFM4 | 2.01 | CD.Fibro.TFPI2.CCL13 | CXCL14 | 2.31 | CD.Prolif/EC.UBE2C.CCNB2 | LSR | 0.63 |
| CD.EC.MT-CO2.MT-CO3 | PIGR | 2.00 | CD.Fibro.TFPI2.CCL13 | CALD1 | 2.29 | CD.Prolif/EC.UBE2C.CCNB2 | ECI1 | 0.63 |
| CD.EC.MT-CO2.MT-CO3 | KRT18 | 1.95 | CD.Fibro.TFPI2.CCL13 | SFRP1 | 2.27 | CD.Prolif/EC.UBE2C.CCNB2 | OTC | 0.62 |
| CD.EC.MT-CO2.MT-CO3 | MT-CO2 | 1.90 | CD.Fibro.TFPI2.CCL13 | TFPI2 | 2.25 | CD.Prolif/EC.UBE2C.CCNB2 | MYO6 | 0.62 |
| CD.EC.MT-CO2.MT-CO3 | KRT8 | 1.87 | CD.Fibro.TFPI2.CCL13 | PLTP | 2.24 | CD.Prolif/EC.UBE2C.CCNB2 | MISP | 0.61 |
| CD.EC.MT-CO2.MT-CO3 | MT-CO3 | 1.83 | CD.Fibro.TFPI2.CCL13 | IGFBP7 | 2.23 | CD.Prolif/EC.UBE2C.CCNB2 | MRPL12 | 0.61 |
| CD.EC.MT-CO2.MT-CO3 | MT1E | 1.80 | CD.Fibro.TFPI2.CCL13 | COL1A2 | 2.20 | CD.Prolif/EC.UBE2C.CCNB2 | NME1 | 0.61 |
| CD.EC.MT-CO2.MT-CO3 | KRT19 | 1.79 | CD.Fibro.TFPI2.CCL13 | GNG11 | 2.16 | CD.Prolif/EC.UBE2C.CCNB2 | BRI3 | 0.61 |
| CD.EC.MT-CO2.MT-CO3 | MT-CYB | 1.72 | CD.Fibro.TFPI2.CCL13 | ADH1B | 2.11 | CD.Prolif/EC.UBE2C.CCNB2 | MTCH2 | 0.61 |
| CD.EC.MT-CO2.MT-CO3 | MT-CO1 | 1.68 | CD.Fibro.TFPI2.CCL13 | CCL8 | 2.08 | CD.Prolif/EC.UBE2C.CCNB2 | VIL1 | 0.61 |
| CD.EC.MT-CO2.MT-CO3 | DMBT1 | 1.66 | CD.Fibro.TFPI2.CCL13 | RBP1 | 2.07 | CD.Prolif/EC.UBE2C.CCNB2 | GOLIM4 | 0.61 |
| CD.EC.MT-CO2.MT-CO3 | TSPAN8 | 1.62 | CD.Fibro.TFPI2.CCL13 | SPARCL1 | 2.06 | CD.Prolif/EC.UBE2C.CCNB2 | CYB5A | 0.61 |
| CD.EC.MT-CO2.MT-CO3 | SPINK1 | 1.59 | CD.Fibro.TFPI2.CCL13 | C1R | 2.00 | CD.Prolif/EC.UBE2C.CCNB2 | GSTA1 | 0.60 |
| CD.EC.MT-CO2.MT-CO3 | MT-ND4 | 1.59 | CD.Fibro.TFPI2.CCL13 | LGALS1 | 1.98 | CD.Prolif/EC.UBE2C.CCNB2 | MAL2 | 0.60 |
| CD.EC.MT-CO2.MT-CO3 | MT-ATP6 | 1.55 | CD.Fibro.TFPI2.CCL13 | GPX3 | 1.97 | CD.Prolif/EC.UBE2C.CCNB2 | TK1 | 0.60 |
| CD.EC.MT-CO2.MT-CO3 | MT-ND2 | 1.50 | CD.Fibro.TFPI2.CCL13 | VCAN | 1.96 | CD.Prolif/EC.UBE2C.CCNB2 | CDH17 | 0.59 |
| CD.EC.MT-CO2.MT-CO3 | PHGR1 | 1.50 | CD.Fibro.TFPI2.CCL13 | COL3A1 | 1.96 | CD.Prolif/EC.UBE2C.CCNB2 | CISD1 | 0.59 |
| CD.EC.MT-CO2.MT-CO3 | PLA2G2A | 1.48 | CD.Fibro.TFPI2.CCL13 | CXCL1 | 1.88 | CD.Prolif/EC.UBE2C.CCNB2 | CCL15 | 0.59 |
| CD.EC.MT-CO2.MT-CO3 | EPCAM | 1.46 | CD.Fibro.TFPI2.CCL13 | CFH | 1.88 | CD.Prolif/EC.UBE2C.CCNB2 | UBE2T | 0.59 |
| CD.EC.MT-CO2.MT-CO3 | GPX2 | 1.41 | CD.Fibro.TFPI2.CCL13 | TMEM176B | 1.87 | CD.Prolif/EC.UBE2C.CCNB2 | CHPT1 | 0.59 |
| CD.EC.MT-CO2.MT-CO3 | LGALS4 | 1.41 | CD.Fibro.TFPI2.CCL13 | NGFRAP1 | 1.84 | CD.Prolif/EC.UBE2C.CCNB2 | TMEM141 | 0.59 |
| CD.EC.MT-CO2.MT-CO3 | CLDN3 | 1.35 | CD.Fibro.TFPI2.CCL13 | S100A13 | 1.81 | CD.Prolif/EC.UBE2C.CCNB2 | MPST | 0.58 |
| CD.EC.MT-CO2.MT-CO3 | PRAP1 | 0.98 | CD.Fibro.TFPI2.CCL13 | TCF21 | 1.79 | CD.Prolif/EC.UBE2C.CCNB2 | USH1C | 0.58 |
| CD.EC.MT-CO2.MT-CO3 | CCL25 | 1.17 | CD.Fibro.TFPI2.CCL13 | PPP1R14A | 1.78 | CD.Prolif/EC.UBE2C.CCNB2 | SMC2 | 0.58 |
| CD.EC.MT-CO2.MT-CO3 | SMIM22 | 1.24 | CD.Fibro.TFPI2.CCL13 | SERPINF1 | 1.77 | CD.Prolif/EC.UBE2C.CCNB2 | RNASE1 | 0.58 |
| CD.EC.MT-CO2.MT-CO3 | AGR2 | 1.12 | CD.Fibro.TFPI2.CCL13 | IGFBP6 | 1.77 | CD.Prolif/EC.UBE2C.CCNB2 | BACE2 | 0.58 |
| CD.EC.MT-CO2.MT-CO3 | C15orf48 | 1.62 | CD.Fibro.TFPI2.CCL13 | PLPP3 | 1.77 | CD.Prolif/EC.UBE2C.CCNB2 | PDHA1 | 0.58 |
| CD.EC.MT-CO2.MT-CO3 | MT-ND5 | 1.52 | CD.Fibro.TFPI2.CCL13 | PDLIM1 | 1.76 | CD.Prolif/EC.UBE2C.CCNB2 | RARRES1 | 0.58 |
| CD.EC.MT-CO2.MT-CO3 | UGT2B17 | 1.06 | CD.Fibro.TFPI2.CCL13 | CXCL6 | 1.70 | CD.Prolif/EC.UBE2C.CCNB2 | HMMR | 0.58 |
| CD.EC.MT1H.MT1G | MT1G | 3.05 | CD.Fibro.TFPI2.CCL13 | TFPI | 1.70 | CD.Prolif/EC.UBE2C.CCNB2 | EI24 | 0.57 |
| CD.EC.MT1H.MT1G | MT1H | 2.77 | CD.Fibro.TFPI2.CCL13 | COL18A1 | 1.70 | CD.Prolif/EC.UBE2C.CCNB2 | PTGR1 | 0.57 |
| CD.EC.MT1H.PHGDH | MT1H | 2.79 | CD.Fibro.TFPI2.CCL13 | FHL2 | 1.69 | CD.Prolif/EC.UBE2C.CCNB2 | GSTM4 | 0.57 |
| CD.EC.MT1H.PHGDH | MT1M | 2.07 | CD.Fibro.TFPI2.CCL13 | MMP2 | 1.67 | CD.Prolif/EC.UBE2C.CCNB2 | CMBL | 0.57 |
| CD.EC.MT1H.PHGDH | UGT2B17 | 1.96 | CD.Fibro.TFPI2.CCL13 | IGF1 | 1.67 | CD.Prolif/EC.UBE2C.CCNB2 | PXMP2 | 0.57 |
| CD.EC.MT1H.PHGDH | KRT20 | 1.87 | CD.Fibro.TFPI2.CCL13 | IFITM3 | 1.65 | CD.Prolif/EC.UBE2C.CCNB2 | TYMS | 0.57 |
| CD.EC.MT1H.PHGDH | AKR1C3 | 1.68 | CD.Fibro.TFPI2.CCL13 | TIMP1 | 1.64 | CD.Prolif/EC.UBE2C.CCNB2 | PBK | 0.57 |
| CD.EC.MT1H.PHGDH | AGR3 | 1.62 | CD.Fibro.TFPI2.CCL13 | GSN | 1.63 | CD.Prolif/EC.UBE2C.CCNB2 | TMEM45B | 0.57 |
| CD.EC.MT1H.PHGDH | S100A14 | 1.30 | CD.Fibro.TFPI2.CCL13 | PMP22 | 1.62 | CD.Prolif/EC.UBE2C.CCNB2 | PPA2 | 0.56 |
| CD.EC.MT1H.PHGDH | TSPAN1 | 1.24 | CD.Fibro.TFPI2.CCL13 | HAPLN1 | 1.59 | CD.Prolif/EC.UBE2C.CCNB2 | LAD1 | 0.56 |
| CD.EC.MT1H.PHGDH | MAOA | 1.12 | CD.Fibro.TFPI2.CCL13 | RAB13 | 1.57 | CD.Prolif/EC.UBE2C.CCNB2 | SMC4 | 0.56 |
| CD.EC.MT1H.PHGDH | TM4SF20 | 1.08 | CD.Fibro.TFPI2.CCL13 | EDIL3 | 1.57 | CD.Prolif/EC.UBE2C.CCNB2 | ST14 | 0.55 |
| CD.EC.MT1H.PHGDH | ADH1C | 1.03 | CD.Fibro.TFPI2.CCL13 | COL6A2 | 1.56 | CD.Prolif/EC.UBE2C.CCNB2 | HSD17B12 | 0.55 |
| CD.EC.MT1H.PHGDH | DHRS11 | 1.03 | CD.Fibro.TFPI2.CCL13 | SPARC | 1.54 | CD.Prolif/EC.UBE2C.CCNB2 | FAM84A | 0.55 |
| CD.EC.MT1H.PHGDH | PRSS3 | 1.01 | CD.Fibro.TFPI2.CCL13 | QSOX1 | 1.54 | CD.Prolif/EC.UBE2C.CCNB2 | MRPS35 | 0.55 |
| CD.EC.MT1H.PHGDH | AKR7A3 | 0.93 | CD.Fibro.TFPI2.CCL13 | TMEM176A | 1.51 | CD.Prolif/EC.UBE2C.CCNB2 | AHCY | 0.55 |
| CD.EC.MT1H.PHGDH | RAB25 | 0.92 | CD.Fibro.TFPI2.CCL13 | GGT5 | 1.50 | CD.Prolif/EC.UBE2C.CCNB2 | PGRMC1 | 0.55 |
| CD.EC.MT1H.PHGDH | TMEM45B | 0.91 | CD.Fibro.TFPI2.CCL13 | SFTA1P | 1.46 | CD.Prolif/EC.UBE2C.CCNB2 | AURKB | 0.55 |
| CD.EC.MT1H.PHGDH | CEACAM5 | 0.89 | CD.Fibro.TFPI2.CCL13 | TGFBI | 1.45 | CD.Prolif/EC.UBE2C.CCNB2 | MAD2L1 | 0.55 |
| CD.EC.MT1H.PHGDH | CDH17 | 0.88 | CD.Fibro.TFPI2.CCL13 | LTBP4 | 1.45 | CD.Prolif/EC.UBE2C.CCNB2 | AP1M2 | 0.55 |
| CD.EC.MT1H.PHGDH | NR1H4 | 0.85 | CD.Fibro.TFPI2.CCL13 | LGALS3BP | 1.45 | CD.Prolif/EC.UBE2C.CCNB2 | ASPM | 0.55 |
| CD.EC.MT1H.PHGDH | MUC1 | 0.81 | CD.Fibro.TFPI2.CCL13 | TIMP3 | 1.42 | CD.Prolif/EC.UBE2C.CCNB2 | NDUFA9 | 0.55 |
| CD.EC.MT1H.PHGDH | USH1C | 0.80 | CD.Fibro.TFPI2.CCL13 | IGFBP4 | 1.42 | CD.Prolif/EC.UBE2C.CCNB2 | C19orf33 | 0.54 |
| CD.EC.MT1H.PHGDH | CMBL | 0.79 | CD.Fibro.TFPI2.CCL13 | ECM1 | 1.42 | CD.Prolif/EC.UBE2C.CCNB2 | CCND1 | 0.54 |
| CD.EC.MT1H.PHGDH | LAD1 | 0.79 | CD.Fibro.TFPI2.CCL13 | CTSK | 1.41 | CD.Prolif/EC.UBE2C.CCNB2 | GGH | 0.54 |
| CD.EC.MT1H.PHGDH | GCNT3 | 0.76 | CD.Fibro.TFPI2.CCL13 | ABCA8 | 1.39 | CD.Prolif/EC.UBE2C.CCNB2 | ZWINT | 0.54 |
| CD.EC.MT1H.PHGDH | C10orf99 | 0.66 | CD.Fibro.TFPI2.CCL13 | SPON2 | 1.39 | CD.Prolif/EC.UBE2C.CCNB2 | EPHB2 | 0.54 |
| CD.EC.MT1H.PHGDH | SULT1E1 | 0.51 | CD.Fibro.TFPI2.CCL13 | NUPR1 | 1.34 | CD.Prolif/EC.UBE2C.CCNB2 | TMEM98 | 0.54 |
| CD.EC.MT1H.PHGDH | AKR1C1 | 0.51 | CD.Fibro.TFPI2.CCL13 | PLAT | 1.33 | CD.Prolif/EC.UBE2C.CCNB2 | MCM7 | 0.54 |
| CD.EC.MT1H.PHGDH | ACKR4 | 0.50 | CD.Fibro.TFPI2.CCL13 | CLEC11A | 1.33 | CD.Prolif/EC.UBE2C.CCNB2 | DTYMK | 0.54 |
| CD.EC.MT1H.PHGDH | SERPINB5 | 0.54 | CD.Fibro.TFPI2.CCL13 | FHL1 | 1.31 | CD.Prolif/EC.UBE2C.CCNB2 | GLRX5 | 0.53 |
| CD.EC.MT1H.PHGDH | MISP | 1.21 | CD.Fibro.TFPI2.CCL13 | SGCE | 1.31 | CD.Prolif/EC.UBE2C.CCNB2 | CENPV | 0.53 |
| CD.EC.MT1H.PHGDH | DMBT1 | 2.17 | CD.Fibro.TFPI2.CCL13 | SERPINE2 | 1.31 | CD.Prolif/EC.UBE2C.CCNB2 | CLDN2 | 0.52 |
| CD.EC.MT1H.PHGDH | RARRES1 | 1.49 | CD.Fibro.TFPI2.CCL13 | EMILIN1 | 1.28 | CD.Prolif/EC.UBE2C.CCNB2 | AP1S1 | 0.52 |
| CD.EC.MT1H.PHGDH | CDC42EP5 | 1.06 | CD.Fibro.TFPI2.CCL13 | FXYD1 | 1.27 | CD.Prolif/EC.UBE2C.CCNB2 | DHFR | 0.52 |
| CD.EC.MT1H.PHGDH | PPP1R1B | 1.03 | CD.Fibro.TFPI2.CCL13 | CYGB | 1.26 | CD.Prolif/EC.UBE2C.CCNB2 | PLEKHJ1 | 0.52 |
| CD.EC.MT1H.PHGDH | MGST1 | 1.37 | CD.Fibro.TFPI2.CCL13 | PLAC9 | 1.23 | CD.Prolif/EC.UBE2C.CCNB2 | TMC5 | 0.52 |
| CD.EC.MT1H.PHGDH | HSD11B2 | 0.73 | CD.Fibro.TFPI2.CCL13 | TPM2 | 1.23 | CD.Prolif/EC.UBE2C.CCNB2 | RCN1 | 0.52 |
| CD.EC.MT1H.PHGDH | FXYD3 | 1.20 | CD.Fibro.TFPI2.CCL13 | RHOBTB3 | 1.21 | CD.Prolif/EC.UBE2C.CCNB2 | CDCA7 | 0.51 |
| CD.EC.MT1H.PHGDH | PLPP2 | 0.80 | CD.Fibro.TFPI2.CCL13 | CLEC14A | 1.20 | CD.Prolif/EC.UBE2C.CCNB2 | SRPK1 | 0.51 |
| CD.EC.MT1H.PHGDH | LAPTM4B | 0.78 | CD.Fibro.TFPI2.CCL13 | PDGFRA | 1.20 | CD.Prolif/EC.UBE2C.CCNB2 | CERS6 | 0.51 |
| CD.EC.MT1H.PHGDH | CES2 | 1.72 | CD.Fibro.TFPI2.CCL13 | FIBIN | 1.19 | CD.Prolif/EC.UBE2C.CCNB2 | RHOB | 0.51 |
| CD.EC.MT1H.PHGDH | CDX1 | 1.01 | CD.Fibro.TFPI2.CCL13 | DDR2 | 1.17 | CD.Prolif/EC.UBE2C.CCNB2 | PLEK2 | 0.51 |
| CD.EC.MT1H.PHGDH | GOLM1 | 0.75 | CD.Fibro.TFPI2.CCL13 | FBLN5 | 1.17 | CD.Prolif/EC.UBE2C.CCNB2 | CASP6 | 0.51 |
| CD.EC.MT1H.PHGDH | KLF5 | 1.34 | CD.Fibro.TFPI2.CCL13 | COL14A1 | 1.17 | CD.Prolif/EC.UBE2C.CCNB2 | HACD3 | 0.51 |
| CD.EC.MTRNR2L1.MT-ND3 | MT-CO3 | 2.47 | CD.Fibro.TFPI2.CCL13 | EDNRB | 1.17 | CD.Prolif/EC.UBE2C.CCNB2 | STAP2 | 0.50 |
| CD.EC.MTRNR2L1.MT-ND3 | MT-ND1 | 2.35 | CD.Fibro.TFPI2.CCL13 | NDN | 1.16 | CD.Prolif/EC.UBE2C.CCNB2 | NBL1 | 0.50 |
| CD.EC.MTRNR2L1.MT-ND3 | MT-CO2 | 2.33 | CD.Fibro.TFPI2.CCL13 | GSTM3 | 1.16 | CD.Prolif/EC.UBE2C.CCNB2 | LCN2 | 0.50 |
| CD.EC.MTRNR2L1.MT-ND3 | MT-CYB | 2.31 | CD.Fibro.TFPI2.CCL13 | PRKCDBP | 1.15 | CD.Prolif/EC.UBE2C.CCNB2 | PKP2 | 0.50 |
| CD.EC.MTRNR2L1.MT-ND3 | MT-CO1 | 2.24 | CD.Fibro.TFPI2.CCL13 | LINC01082 | 1.13 | CD.Prolif/EC.UBE2C.CCNB2 | TM9SF3 | 0.50 |
| CD.EC.MTRNR2L1.MT-ND3 | MT-ND3 | 2.21 | CD.Fibro.TFPI2.CCL13 | OLFML3 | 1.12 | CD.Prolif/EC.UBE2C.CCNB2 | CHP2 | 0.50 |
| CD.EC.MTRNR2L1.MT-ND3 | MT-ND4L | 2.18 | CD.Fibro.TFPI2.CCL13 | TMEM98 | 1.12 | CD.Prolif/EC.UBE2C.CCNB2 | SERPINB5 | 0.50 |
| CD.EC.MTRNR2L1.MT-ND3 | MT-ND4 | 2.18 | CD.Fibro.TFPI2.CCL13 | CXCL12 | 1.11 | CD.Prolif/EC.UBE2C.CCNB2 | CCNA2 | 0.50 |
| CD.EC.MTRNR2L1.MT-ND3 | MT-ND2 | 2.17 | CD.Fibro.TFPI2.CCL13 | RBP5 | 1.11 | CD.Prolif/EC.UBE2C.CCNB2 | TCEA3 | 0.50 |
| CD.EC.MTRNR2L1.MT-ND3 | MT-ATP6 | 2.14 | CD.Fibro.TFPI2.CCL13 | C1orf21 | 1.09 | CD.Prolif/EC.UBE2C.CCNB2 | SUCLG2 | 0.50 |
| CD.EC.MTRNR2L1.MT-ND3 | MT-ND5 | 2.08 | CD.Fibro.TFPI2.CCL13 | NID1 | 1.09 | CD.Prolif/EC.UBE2C.CCNB2 | HNF4A | 0.50 |
| CD.EC.MTRNR2L1.MT-ND3 | MTRNR2L12 | 1.87 | CD.Fibro.TFPI2.CCL13 | DPT | 1.07 | CD.Prolif/EC.UBE2C.CCNB2 | TPX2 | 0.49 |
| CD.EC.MTRNR2L1.MT-ND3 | OLFM4 | 1.84 | CD.Fibro.TFPI2.CCL13 | COL6A3 | 1.06 | CD.Prolif/EC.UBE2C.CCNB2 | EIF4EBP1 | 0.49 |
| CD.EC.MTRNR2L1.MT-ND3 | REG1A | 1.58 | CD.Fibro.TFPI2.CCL13 | MYL9 | 1.06 | CD.Prolif/EC.UBE2C.CCNB2 | PAICS | 0.49 |
| CD.EC.MTRNR2L1.MT-ND3 | MT-ND6 | 1.45 | CD.Fibro.TFPI2.CCL13 | BMP4 | 1.06 | CD.Prolif/EC.UBE2C.CCNB2 | ACTN4 | 0.49 |
| CD.EC.MTRNR2L1.MT-ND3 | PIGR | 1.45 | CD.Fibro.TFPI2.CCL13 | RBPMS | 1.06 | CD.Prolif/EC.UBE2C.CCNB2 | ACOT7 | 0.49 |
| CD.EC.MTRNR2L1.MT-ND3 | MTRNR2L8 | 1.45 | CD.Fibro.TFPI2.CCL13 | EFEMP2 | 1.06 | CD.Prolif/EC.UBE2C.CCNB2 | CDH1 | 0.49 |
| CD.EC.MTRNR2L1.MT-ND3 | KRT18 | 1.39 | CD.Fibro.TFPI2.CCL13 | SCARA5 | 1.06 | CD.Prolif/EC.UBE2C.CCNB2 | DCTPP1 | 0.49 |
| CD.EC.MTRNR2L1.MT-ND3 | MT-ATP8 | 1.35 | CD.Fibro.TFPI2.CCL13 | DKK3 | 1.05 | CD.Prolif/EC.UBE2C.CCNB2 | CEACAM5 | 0.49 |
| CD.EC.MTRNR2L1.MT-ND3 | SPINK1 | 1.30 | CD.Fibro.TFPI2.CCL13 | COL15A1 | 1.04 | CD.Prolif/EC.UBE2C.CCNB2 | NAPRT | 0.49 |
| CD.EC.MTRNR2L1.MT-ND3 | KRT8 | 1.29 | CD.Fibro.TFPI2.CCL13 | NNMT | 1.01 | CD.Prolif/EC.UBE2C.CCNB2 | TOMM40 | 0.49 |
| CD.EC.MTRNR2L1.MT-ND3 | DMBT1 | 1.25 | CD.Fibro.TFPI2.CCL13 | COL6A1 | 0.99 | CD.Prolif/EC.UBE2C.CCNB2 | AKAP1 | 0.48 |
| CD.EC.MTRNR2L1.MT-ND3 | MTRNR2L1 | 1.13 | CD.Fibro.TFPI2.CCL13 | EFEMP1 | 0.98 | CD.Prolif/EC.UBE2C.CCNB2 | CAMK2N1 | 0.48 |
| CD.EC.MTRNR2L1.MT-ND3 | TSPAN8 | 1.11 | CD.Fibro.TFPI2.CCL13 | C7 | 0.98 | CD.Prolif/EC.UBE2C.CCNB2 | CNN3 | 0.48 |
| CD.EC.MTRNR2L1.MT-ND3 | ELF3 | 1.03 | CD.Fibro.TFPI2.CCL13 | SNAI2 | 0.97 | CD.Prolif/EC.UBE2C.CCNB2 | AKR7A3 | 0.48 |
| CD.EC.MTRNR2L1.MT-ND3 | GPX2 | 1.02 | CD.Fibro.TFPI2.CCL13 | MEG3 | 0.96 | CD.Prolif/EC.UBE2C.CCNB2 | TSC22D1 | 0.48 |
| CD.EC.MTRNR2L1.MT-ND3 | CLDN4 | 1.01 | CD.Fibro.TFPI2.CCL13 | PAPPA | 0.95 | CD.Prolif/EC.UBE2C.CCNB2 | IFT172 | 0.47 |
| CD.EC.MTRNR2L1.MT-ND3 | MT1E | 1.01 | CD.Fibro.TFPI2.CCL13 | LAMA4 | 0.93 | CD.Prolif/EC.UBE2C.CCNB2 | ASL | 0.47 |
| CD.EC.MTRNR2L1.MT-ND3 | MT1G | 0.97 | CD.Fibro.TFPI2.CCL13 | STMN2 | 0.92 | CD.Prolif/EC.UBE2C.CCNB2 | TSPAN6 | 0.47 |
| CD.EC.MTRNR2L1.MT-ND3 | KRT19 | 0.96 | CD.Fibro.TFPI2.CCL13 | PCDH18 | 0.90 | CD.Prolif/EC.UBE2C.CCNB2 | HOXB7 | 0.47 |
| CD.EC.MTRNR2L1.MT-ND3 | EPCAM | 0.94 | CD.Fibro.TFPI2.CCL13 | ANGPTL1 | 0.90 | CD.Prolif/EC.UBE2C.CCNB2 | CENPA | 0.47 |
| CD.EC.MTRNR2L1.MT-ND3 | LGALS4 | 0.91 | CD.Fibro.TFPI2.CCL13 | HOXC9 | 0.87 | CD.Prolif/EC.UBE2C.CCNB2 | PKP3 | 0.47 |
| CD.EC.MTRNR2L1.MT-ND3 | CLDN3 | 0.86 | CD.Fibro.TFPI2.CCL13 | LTBP1 | 0.85 | CD.Prolif/EC.UBE2C.CCNB2 | CTBP2 | 0.47 |
| CD.EC.MTRNR2L1.MT-ND3 | PHGR1 | 0.80 | CD.Fibro.TFPI2.CCL13 | LINC01116 | 0.84 | CD.Prolif/EC.UBE2C.CCNB2 | BOLA3 | 0.47 |
| CD.EC.MTRNR2L1.MT-ND3 | PLA2G2A | 0.44 | CD.Fibro.TFPI2.CCL13 | AKAP12 | 0.84 | CD.Prolif/EC.UBE2C.CCNB2 | IMPA2 | 0.47 |
| CD.EC.MTRNR2L1.MT-ND3 | PRAP1 | 0.39 | CD.Fibro.TFPI2.CCL13 | TNFAIP6 | 0.80 | CD.Prolif/EC.UBE2C.CCNB2 | LAPTM4B | 0.46 |
| CD.EC.MTRNR2L1.MT-ND3 | AGR2 | 0.55 | CD.Fibro.TFPI2.CCL13 | IL34 | 0.80 | CD.Prolif/EC.UBE2C.CCNB2 | TST | 0.46 |
| CD.EC.MTRNR2L1.MT-ND3 | SMIM22 | 0.75 | CD.Fibro.TFPI2.CCL13 | GLT8D2 | 0.75 | CD.Prolif/EC.UBE2C.CCNB2 | PLGRKT | 0.46 |
| CD.EC.NUPR1.LCN2 | UGT2B17 | 1.70 | CD.Fibro.TFPI2.CCL13 | MXRA8 | 0.74 | CD.Prolif/EC.UBE2C.CCNB2 | MRPL13 | 0.46 |
| CD.EC.OLFM4.MT-ND2 | REG3A | 2.07 | CD.Fibro.TFPI2.CCL13 | NEGR1 | 0.72 | CD.Prolif/EC.UBE2C.CCNB2 | NFIB | 0.46 |
| CD.EC.PLCG2.MAFB | PLCG2 | 4.29 | CD.Fibro.TFPI2.CCL13 | TSPAN2 | 0.70 | CD.Prolif/EC.UBE2C.CCNB2 | MSRB2 | 0.45 |
| CD.EC.PLCG2.MAFB | ELF3 | 3.08 | CD.Fibro.TFPI2.CCL13 | HGF | 0.69 | CD.Prolif/EC.UBE2C.CCNB2 | DPEP1 | 0.45 |
| CD.EC.PLCG2.MAFB | SOX4 | 2.64 | CD.Fibro.TFPI2.CCL13 | PTGES | 0.69 | CD.Prolif/EC.UBE2C.CCNB2 | ERBB3 | 0.44 |
| CD.EC.PLCG2.MTRNR2L8 | PLCG2 | 3.64 | CD.Fibro.TFPI2.CCL13 | ANGPTL2 | 0.67 | CD.Prolif/EC.UBE2C.CCNB2 | CKMT1B | 0.44 |
| CD.EC.PLCG2.MTRNR2L8 | MTRNR2L8 | 3.21 | CD.Fibro.TFPI2.CCL13 | TMEM150C | 0.65 | CD.Prolif/EC.UBE2C.CCNB2 | PTPRF | 0.44 |
| CD.EC.PLCG2.MTRNR2L8 | APOA1 | 0.80 | CD.Fibro.TFPI2.CCL13 | ELANE | 0.56 | CD.Prolif/EC.UBE2C.CCNB2 | C2orf82 | 0.44 |
| CD.EC.PLCG2.MTRNR2L8 | RND3 | 1.85 | CD.Fibro.TFPI2.CCL13 | COLEC10 | 0.52 | CD.Prolif/EC.UBE2C.CCNB2 | FBP1 | 0.44 |
| CD.EC.PLCG2.RP11-727F159 | PLCG2 | 2.68 | CD.Fibro.TFPI2.CCL13 | FSTL1 | 0.97 | CD.Prolif/EC.UBE2C.CCNB2 | CKMT1A | 0.44 |
| CD.EC.PLCG2.RP11-727F159 | MT1G | 2.31 | CD.Fibro.TFPI2.CCL13 | KCNS3 | 0.68 | CD.Prolif/EC.UBE2C.CCNB2 | PRSS8 | 0.44 |
| CD.EC.PLCG2.RP11-727F159 | ELF3 | 2.15 | CD.Fibro.TFPI2.CCL13 | GRK5 | 0.84 | CD.Prolif/EC.UBE2C.CCNB2 | PRC1 | 0.44 |
| CD.EC.PLCG2.RP11-727F159 | MT1E | 2.05 | CD.Fibro.TFPI2.CCL13 | CLMP | 0.81 | CD.Prolif/EC.UBE2C.CCNB2 | PON2 | 0.44 |
| CD.EC.PLCG2.RP11-727F159 | PHGR1 | 1.93 | CD.Fibro.TFPI2.CCL13 | PCDH7 | 0.68 | CD.Prolif/EC.UBE2C.CCNB2 | HELLS | 0.44 |
| CD.EC.PLCG2.RP11-727F159 | REG1A | 1.91 | CD.Fibro.TFPI2.CCL13 | ABCA6 | 0.75 | CD.Prolif/EC.UBE2C.CCNB2 | CTNNA1 | 0.44 |
| CD.EC.PLCG2.RP11-727F159 | KRT18 | 1.89 | CD.Fibro.TFPI2.CCL13 | GJA1 | 0.74 | CD.Prolif/EC.UBE2C.CCNB2 | DDC | 0.43 |
| CD.EC.PLCG2.RP11-727F159 | C19orf33 | 1.78 | CD.Fibro.TFPI2.CCL13 | SAMD11 | 0.69 | CD.Prolif/EC.UBE2C.CCNB2 | PLCB3 | 0.43 |
| CD.EC.PLCG2.RP11-727F159 | KRT8 | 1.70 | CD.Fibro.TFPI2.CCL13 | NOVA1 | 0.64 | CD.Prolif/EC.UBE2C.CCNB2 | LLGL2 | 0.43 |
| CD.EC.PLCG2.RP11-727F159 | SPINK1 | 1.63 | CD.Fibro.TFPI2.CCL13 | SELM | 1.49 | CD.Prolif/EC.UBE2C.CCNB2 | MRPS25 | 0.43 |
| CD.EC.PLCG2.RP11-727F159 | PRAP1 | 1.31 | CD.Fibro.TFPI2.CCL13 | CDH11 | 0.77 | CD.Prolif/EC.UBE2C.CCNB2 | MUC1 | 0.43 |
| CD.EC.PLCG2.RP11-727F159 | REG1B | 1.73 | CD.Fibro.TFPI2.CCL13 | ANGPTL4 | 0.73 | CD.Prolif/EC.UBE2C.CCNB2 | GOT1 | 0.43 |
| CD.EC.RBP2.ALPI | REG1B | 3.93 | CD.Fibro.TNFRSF11B.TFPI2 | CCL13 | 3.58 | CD.Prolif/EC.UBE2C.CCNB2 | TMEM97 | 0.43 |
| CD.EC.RBP2.ALPI | RBP2 | 3.69 | CD.Fibro.TNFRSF11B.TFPI2 | IGFBP5 | 3.52 | CD.Prolif/EC.UBE2C.CCNB2 | STXBP6 | 0.43 |
| CD.EC.RBP2.ALPI | ALDOB | 3.58 | CD.Fibro.TNFRSF11B.TFPI2 | CXCL6 | 3.22 | CD.Prolif/EC.UBE2C.CCNB2 | HMGCS2 | 0.43 |
| CD.EC.RBP2.ALPI | FABP1 | 3.43 | CD.Fibro.TNFRSF11B.TFPI2 | TFPI2 | 3.09 | CD.Prolif/EC.UBE2C.CCNB2 | DLGAP5 | 0.42 |
| CD.EC.RBP2.ALPI | PRAP1 | 3.33 | CD.Fibro.TNFRSF11B.TFPI2 | ADH1B | 2.39 | CD.Prolif/EC.UBE2C.CCNB2 | COA4 | 0.42 |
| CD.EC.RBP2.ALPI | FABP2 | 2.89 | CD.Fibro.TNFRSF11B.TFPI2 | MMP1 | 2.12 | CD.Prolif/EC.UBE2C.CCNB2 | QTRT1 | 0.42 |
| CD.EC.RBP2.ALPI | ANPEP | 2.79 | CD.Fibro.TNFRSF11B.TFPI2 | TCF21 | 1.93 | CD.Prolif/EC.UBE2C.CCNB2 | APP | 0.42 |
| CD.EC.RBP2.ALPI | FABP6 | 2.71 | CD.Fibro.TNFRSF11B.TFPI2 | SFRP1 | 1.93 | CD.Prolif/EC.UBE2C.CCNB2 | CRYL1 | 0.41 |
| CD.EC.RBP2.ALPI | GUCAZA | 2.67 | CD.Fibro.TNFRSF11B.TFPI2 | EMILIN1 | 1.90 | CD.Prolif/EC.UBE2C.CCNB2 | SLC44A4 | 0.41 |
| CD.EC.RBP2.ALPI | MT1H | 2.60 | CD.Fibro.TNFRSF11B.TFPI2 | CYGB | 1.89 | CD.Prolif/EC.UBE2C.CCNB2 | HSD11B2 | 0.41 |
| CD.EC.RBP2.ALPI | SMIM24 | 2.57 | CD.Fibro.TNFRSF11B.TFPI2 | TNFRSF11B | 1.88 | CD.Prolif/EC.UBE2C.CCNB2 | DDAH1 | 0.41 |
| CD.EC.RBP2.ALPI | CDHR5 | 2.25 | CD.Fibro.TNFRSF11B.TFPI2 | FIBIN | 1.86 | CD.Prolif/EC.UBE2C.CCNB2 | SH2D4A | 0.41 |
| CD.EC.RBP2.ALPI | PCK1 | 1.78 | CD.Fibro.TNFRSF11B.TFPI2 | FGF7 | 1.84 | CD.Prolif/EC.UBE2C.CCNB2 | MRPL15 | 0.41 |
| CD.EC.RBP2.ALPI | APOB | 1.72 | CD.Fibro.TNFRSF11B.TFPI2 | OLFML3 | 1.77 | CD.Prolif/EC.UBE2C.CCNB2 | GMNN | 0.41 |
| CD.EC.RBP2.ALPI | SLC5A1 | 1.67 | CD.Fibro.TNFRSF11B.TFPI2 | GGT5 | 1.75 | CD.Prolif/EC.UBE2C.CCNB2 | MYH14 | 0.41 |
| CD.EC.RBP2.ALPI | ALPI | 1.60 | CD.Fibro.TNFRSF11B.TFPI2 | STEAP1 | 1.60 | CD.Prolif/EC.UBE2C.CCNB2 | HMGN5 | 0.41 |
| CD.EC.RBP2.ALPI | FLJ22763 | 1.37 | CD.Fibro.TNFRSF11B.TFPI2 | HGF | 1.43 | CD.Prolif/EC.UBE2C.CCNB2 | FAM195A | 0.40 |
| CD.EC.RBP2.ALPI | FAM132A | 1.22 | CD.Fibro.TNFRSF11B.TFPI2 | PDGFRA | 1.43 | CD.Prolif/EC.UBE2C.CCNB2 | RARS | 0.40 |
| CD.EC.RBP2.ALPI | C11orf86 | 1.05 | CD.Fibro.TNFRSF11B.TFPI2 | PDPN | 1.41 | CD.Prolif/EC.UBE2C.CCNB2 | MLLT4 | 0.40 |
| CD.EC.RBP2.ALPI | SERPINA1 | 2.72 | CD.Fibro.TNFRSF11B.TFPI2 | LINC01082 | 1.36 | CD.Prolif/EC.UBE2C.CCNB2 | EPS8L3 | 0.40 |
| CD.EC.RBP2.ALPI | ADIRF | 2.47 | CD.Fibro.TNFRSF11B.TFPI2 | SFTA1P | 1.31 | CD.Prolif/EC.UBE2C.CCNB2 | CBLC | 0.40 |
| CD.EC.RBP2.ALPI | MT1G | 2.95 | CD.Fibro.TNFRSF11B.TFPI2 | MEG3 | 1.22 | CD.Prolif/EC.UBE2C.CCNB2 | PAWR | 0.40 |
| CD.EC.RBP2.ALPI | ERICH4 | 0.92 | CD.Fibro.TNFRSF11B.TFPI2 | CDH11 | 1.22 | CD.Prolif/EC.UBE2C.CCNB2 | WDR34 | 0.40 |
| CD.EC.RBP2.ALPI | SMLR1 | 0.65 | CD.Fibro.TNFRSF11B.TFPI2 | FBN1 | 1.15 | CD.Prolif/EC.UBE2C.CCNB2 | BZW2 | 0.40 |
| CD.EC.RBP2.ALPI | TM6SF2 | 1.08 | CD.Fibro.TNFRSF11B.TFPI2 | SULF1 | 1.14 | CD.Prolif/EC.UBE2C.CCNB2 | TM4SF20 | 0.40 |
| CD.EC.RBP2.ALPI | CDHR2 | 1.21 | CD.Fibro.TNFRSF11B.TFPI2 | COL14A1 | 1.11 | CD.Prolif/EC.UBE2C.CCNB2 | UGDH | 0.40 |
| CD.EC.RBP2.CYP3A4 | RBP2 | 3.67 | CD.Fibro.TNFRSF11B.TFPI2 | PTGES | 1.10 | CD.Prolif/EC.UBE2C.CCNB2 | GGT6 | 0.40 |
| CD.EC.RBP2.CYP3A4 | FABP6 | 3.62 | CD.Fibro.TNFRSF11B.TFPI2 | ANGPTL2 | 1.05 | CD.Prolif/EC.UBE2C.CCNB2 | HEBP1 | 0.40 |
| CD.EC.RBP2.CYP3A4 | PRAP1 | 3.61 | CD.Fibro.TNFRSF11B.TFPI2 | PAPPA | 0.96 | CD.Prolif/EC.UBE2C.CCNB2 | ABHD11 | 0.40 |
| CD.EC.RBP2.CYP3A4 | FABP2 | 3.59 | CD.Fibro.TNFRSF11B.TFPI2 | FMOD | 0.95 | CD.Prolif/EC.UBE2C.CCNB2 | RRM1 | 0.40 |
| CD.EC.RBP2.CYP3A4 | ALDOB | 3.58 | CD.Fibro.TNFRSF11B.TFPI2 | GABRA2 | 0.95 | CD.Prolif/EC.UBE2C.CCNB2 | SERINC2 | 0.39 |
| CD.EC.RBP2.CYP3A4 | FABP1 | 3.48 | CD.Fibro.TNFRSF11B.TFPI2 | SAMD11 | 0.95 | CD.Prolif/EC.UBE2C.CCNB2 | STARD10 | 0.39 |
| CD.EC.RBP2.CYP3A4 | ADIRE | 3.43 | CD.Fibro.TNFRSF11B.TFPI2 | HTRA3 | 0.86 | CD.Prolif/EC.UBE2C.CCNB2 | MRPL37 | 0.39 |
| CD.EC.RBP2.CYP3A4 | ANPEP | 2.92 | CD.Fibro.TNFRSF11B.TFPI2 | COLEC10 | 0.85 | CD.Prolif/EC.UBE2C.CCNB2 | TRAP1 | 0.39 |
| CD.EC.RBP2.CYP3A4 | GUCAZA | 2.92 | CD.Fibro.TNFRSF11B.TFPI2 | HSD11B1 | 0.84 | CD.Prolif/EC.UBE2C.CCNB2 | PPP1R14B | 0.39 |
| CD.EC.RBP2.CYP3A4 | CDHR5 | 2.54 | CD.Fibro.TNFRSF11B.TFPI2 | CCL7 | 0.77 | CD.Prolif/EC.UBE2C.CCNB2 | RUVBL2 | 0.39 |
| CD.EC.RBP2.CYP3A4 | SMIM24 | 2.43 | CD.Fibro.TNFRSF11B.TFPI2 | MRGPRF | 0.74 | CD.Prolif/EC.UBE2C.CCNB2 | RAB13 | 0.39 |
| CD.EC.RBP2.CYP3A4 | GSTA1 | 2.43 | CD.Fibro.TNFRSF11B.TFPI2 | BDKRB1 | 0.66 | CD.Prolif/EC.UBE2C.CCNB2 | TRIM2 | 0.39 |
| CD.EC.RBP2.CYP3A4 | SI | 2.18 | CD.Fibro.TNFRSF11B.TFPI2 | RP6-65G23.5 | 0.65 | CD.Prolif/EC.UBE2C.CCNB2 | IDH1 | 0.39 |
| CD.EC.RBP2.CYP3A4 | APOB | 2.15 | CD.Fibro.TNFRSF11B.TFPI2 | APELA | 0.56 | CD.Prolif/EC.UBE2C.CCNB2 | SLC39A5 | 0.39 |
| CD.EC.RBP2.CYP3A4 | AMN | 2.13 | CD.Fibro.TNFRSF11B.TFPI2 | IL13RA2 | 0.56 | CD.Prolif/EC.UBE2C.CCNB2 | ZBTB7A | 0.39 |
| CD.EC.RBP2.CYP3A4 | MTTP | 2.04 | CD.Fibro.TNFRSF11B.TFPI2 | FGF10 | 0.55 | CD.Prolif/EC.UBE2C.CCNB2 | TRIM28 | 0.39 |
| CD.EC.RBP2.CYP3A4 | KRT20 | 2.04 | CD.Fibro.TNFRSF11B.TFPI2 | TSPAN2 | 1.01 | CD.Prolif/EC.UBE2C.CCNB2 | LINC01207 | 0.38 |
| CD.EC.RBP2.CYP3A4 | DGAT1 | 2.02 | CD.Fibro.TNFRSF11B.TFPI2 | COL6A3 | 1.72 | CD.Prolif/EC.UBE2C.CCNB2 | GCNT3 | 0.38 |
| CD.EC.RBP2.CYP3A4 | ACE | 1.93 | CD.Fibro.TNFRSF11B.TFPI2 | EFEMP1 | 1.21 | CD.Prolif/EC.UBE2C.CCNB2 | SPINT1 | 0.38 |
| CD.EC.RBP2.CYP3A4 | TM4SF20 | 1.77 | CD.Fibro.TNFRSF11B.TFPI2 | CCL11 | 4.57 | CD.Prolif/EC.UBE2C.CCNB2 | AMN | 0.38 |
| CD.EC.RBP2.CYP3A4 | SLC5A1 | 1.76 | CD.Fibro.TNFRSF11B.TFPI2 | FBLN1 | 2.38 | CD.Prolif/EC.UBE2C.CCNB2 | CTTN | 0.38 |
| CD.EC.RBP2.CYP3A4 | MEP1A | 1.74 | CD.Fibro.TNFRSF11B.TFPI2 | WISP1 | 0.29 | CD.Prolif/EC.UBE2C.CCNB2 | GINS2 | 0.38 |
| CD.EC.RBP2.CYP3A4 | MYO1A | 1.71 | CD.Fibro.TNFRSF11B.TFPI2 | ECM1 | 1.29 | CD.Prolif/EC.UBE2C.CCNB2 | MAPK13 | 0.38 |
| CD.EC.RBP2.CYP3A4 | FLJ22763 | 1.62 | CD.Fibro.TNFRSF11B.TFPI2 | PTGFR | 0.62 | CD.Prolif/EC.UBE2C.CCNB2 | HDHD3 | 0.38 |
| CD.EC.RBP2.CYP3A4 | MALL | 1.62 | CD.Fibro.TNFRSF11B.TFPI2 | MMP2 | 2.25 | CD.Prolif/EC.UBE2C.CCNB2 | PLK1 | 0.38 |
| CD.EC.RBP2.CYP3A4 | ALPI | 1.58 | CD.Fibro.TNFRSF11B.TFPI2 | COL18A1 | 2.69 | CD.Prolif/EC.UBE2C.CCNB2 | PRR15L | 0.38 |
| CD.EC.RBP2.CYP3A4 | ENPEP | 1.55 | CD.Fibro.TNFRSF11B.TFPI2 | SLC16A4 | 1.06 | CD.Prolif/EC.UBE2C.CCNB2 | EHF | 0.38 |
| CD.EC.RBP2.CYP3A4 | AOC1 | 1.52 | CD.Fibro.TNFSF11.CD24 | CXCL14 | 3.63 | CD.Prolif/EC.UBE2C.CCNB2 | CLRN3 | 0.37 |
| CD.EC.RBP2.CYP3A4 | ACE2 | 1.46 | CD.Fibro.TNFSF11.CD24 | CCL19 | 3.10 | CD.Prolif/EC.UBE2C.CCNB2 | NUF2 | 0.37 |
| CD.EC.RBP2.CYP3A4 | CYP3A4 | 1.45 | CD.Fibro.TNFSF11.CD24 | RARRES2 | 2.83 | CD.Prolif/EC.UBE2C.CCNB2 | ACSL5 | 0.37 |
| CD.EC.RBP2.CYP3A4 | SLC15A1 | 1.44 | CD.Fibro.TNFSF11.CD24 | C1S | 2.71 | CD.Prolif/EC.UBE2C.CCNB2 | ASRGL1 | 0.37 |
| CD.EC.RBP2.CYP3A4 | C11orf86 | 1.43 | CD.Fibro.TNFSF11.CD24 | C3 | 2.56 | CD.Prolif/EC.UBE2C.CCNB2 | ACADS | 0.37 |
| CD.EC.RBP2.CYP3A4 | SLC26A3 | 1.43 | CD.Fibro.TNFSF11.CD24 | C1R | 2.47 | CD.Prolif/EC.UBE2C.CCNB2 | CFTR | 0.37 |
| CD.EC.RBP2.CYP3A4 | CDHR2 | 1.41 | CD.Fibro.TNFSF11.CD24 | IGFBP7 | 2.45 | CD.Prolif/EC.UBE2C.CCNB2 | GJB1 | 0.37 |
| CD.EC.RBP2.CYP3A4 | SULT1A2 | 1.35 | CD.Fibro.TNFSF11.CD24 | CALD1 | 2.34 | CD.Prolif/EC.UBE2C.CCNB2 | MYO10 | 0.37 |
| CD.EC.RBP2.CYP3A4 | MME | 1.33 | CD.Fibro.TNFSF11.CD24 | DCN | 2.29 | CD.Prolif/EC.UBE2C.CCNB2 | ATP8B1 | 0.37 |
| CD.EC.RBP2.CYP3A4 | MGAM | 1.30 | CD.Fibro.TNFSF11.CD24 | LUM | 2.23 | CD.Prolif/EC.UBE2C.CCNB2 | ALDH7A1 | 0.37 |
| CD.EC.RBP2.CYP3A4 | AKR1B10 | 1.29 | CD.Fibro.TNFSF11.CD24 | S100A13 | 2.14 | CD.Prolif/EC.UBE2C.CCNB2 | SPC25 | 0.37 |
| CD.EC.RBP2.CYP3A4 | LINC01133 | 1.29 | CD.Fibro.TNFSF11.CD24 | SERPINF1 | 2.10 | CD.Prolif/EC.UBE2C.CCNB2 | GIPC2 | 0.37 |
| CD.EC.RBP2.CYP3A4 | SLC6A19 | 1.27 | CD.Fibro.TNFSF11.CD24 | TMEM176B | 2.10 | CD.Prolif/EC.UBE2C.CCNB2 | PDZK1IP1 | 0.37 |
| CD.EC.RBP2.CYP3A4 | XPNPEP2 | 1.24 | CD.Fibro.TNFSF11.CD24 | SOD3 | 2.03 | CD.Prolif/EC.UBE2C.CCNB2 | CXADR | 0.37 |
| CD.EC.RBP2.CYP3A4 | SULT1B1 | 1.22 | CD.Fibro.TNFSF11.CD24 | SERPING1 | 2.02 | CD.Prolif/EC.UBE2C.CCNB2 | AHCYL1 | 0.37 |
| CD.EC.RBP2.CYP3A4 | MS4A8 | 1.19 | CD.Fibro.TNFSF11.CD24 | TMEM176A | 1.89 | CD.Prolif/EC.UBE2C.CCNB2 | EPS8 | 0.37 |
| CD.EC.RBP2.CYP3A4 | CREB3L3 | 1.18 | CD.Fibro.TNFSF11.CD24 | ADAMDEC1 | 1.88 | CD.Prolif/EC.UBE2C.CCNB2 | DSC2 | 0.36 |
| CD.EC.RBP2.CYP3A4 | SLC7A9 | 1.18 | CD.Fibro.TNFSF11.CD24 | CTSK | 1.87 | CD.Prolif/EC.UBE2C.CCNB2 | SOX9 | 0.36 |
| CD.EC.RBP2.CYP3A4 | SLC51B | 1.16 | CD.Fibro.TNFSF11.CD24 | COL6A2 | 1.86 | CD.Prolif/EC.UBE2C.CCNB2 | MLXIP | 0.36 |
| CD.EC.RBP2.CYP3A4 | FAM132A | 1.15 | CD.Fibro.TNFSF11.CD24 | TPM2 | 1.85 | CD.Prolif/EC.UBE2C.CCNB2 | CDCA3 | 0.36 |
| CD.EC.RBP2.CYP3A4 | TMIGD1 | 1.14 | CD.Fibro.TNFSF11.CD24 | PCOLCE | 1.84 | CD.Prolif/EC.UBE2C.CCNB2 | MUC13 | 0.36 |
| CD.EC.RBP2.CYP3A4 | SLC10A2 | 1.10 | CD.Fibro.TNFSF11.CD24 | TAGLN | 1.82 | CD.Prolif/EC.UBE2C.CCNB2 | GTSE1 | 0.36 |
| CD.EC.RBP2.CYP3A4 | GDA | 1.09 | CD.Fibro.TNFSF11.CD24 | NBL1 | 1.80 | CD.Prolif/EC.UBE2C.CCNB2 | SORD | 0.36 |
| CD.EC.RBP2.CYP3A4 | SCIN | 1.08 | CD.Fibro.TNFSF11.CD24 | NNMT | 1.78 | CD.Prolif/EC.UBE2C.CCNB2 | PGM1 | 0.36 |
| CD.EC.RBP2.CYP3A4 | ABHD11-AS1 | 1.02 | CD.Fibro.TNFSF11.CD24 | MFAP4 | 1.76 | CD.Prolif/EC.UBE2C.CCNB2 | SLC25A1 | 0.36 |
| CD.EC.RBP2.CYP3A4 | MOGAT2 | 1.02 | CD.Fibro.TNFSF11.CD24 | TDO2 | 1.71 | CD.Prolif/EC.UBE2C.CCNB2 | CDS1 | 0.35 |
| CD.EC.RBP2.CYP3A4 | HHLA2 | 1.02 | CD.Fibro.TNFSF11.CD24 | CHI3L1 | 1.68 | CD.Prolif/EC.UBE2C.CCNB2 | CEP55 | 0.35 |
| CD.EC.RBP2.CYP3A4 | SLC3A1 | 1.02 | CD.Fibro.TNFSF11.CD24 | ACTA2 | 1.68 | CD.Prolif/EC.UBE2C.CCNB2 | PPP1R14D | 0.35 |
| CD.EC.RBP2.CYP3A4 | AADAC | 1.01 | CD.Fibro.TNFSF11.CD24 | VCAM1 | 1.66 | CD.Prolif/EC.UBE2C.CCNB2 | BRI3BP | 0.35 |
| CD.EC.RBP2.CYP3A4 | ESPN | 1.01 | CD.Fibro.TNFSF11.CD24 | FILIP1L | 1.64 | CD.Prolif/EC.UBE2C.CCNB2 | MGAT4B | 0.35 |
| CD.EC.RBP2.CYP3A4 | XDH | 1.00 | CD.Fibro.TNFSF11.CD24 | MYL9 | 1.64 | CD.Prolif/EC.UBE2C.CCNB2 | KCNE3 | 0.34 |
| CD.EC.RBP2.CYP3A4 | C17orf78 | 0.92 | CD.Fibro.TNFSF11.CD24 | RBP5 | 1.61 | CD.Prolif/EC.UBE2C.CCNB2 | CENPN | 0.34 |
| CD.EC.RBP2.CYP3A4 | GSTA2 | 0.87 | CD.Fibro.TNFSF11.CD24 | COL3A1 | 1.61 | CD.Prolif/EC.UBE2C.CCNB2 | MARVELD3 | 0.34 |
| CD.EC.RBP2.CYP3A4 | TREH | 0.85 | CD.Fibro.TNFSF11.CD24 | PLPP1 | 1.61 | CD.Prolif/EC.UBE2C.CCNB2 | ILVBL | 0.34 |
| CD.EC.RBP2.CYP3A4 | MS4A10 | 0.83 | CD.Fibro.TNFSF11.CD24 | NUPR1 | 1.60 | CD.Prolif/EC.UBE2C.CCNB2 | AQP1 | 0.34 |
| CD.EC.RBP2.CYP3A4 | TM6SF2 | 0.82 | CD.Fibro.TNFSF11.CD24 | COL1A2 | 1.55 | CD.Prolif/EC.UBE2C.CCNB2 | GSDMB | 0.33 |
| CD.EC.RBP2.CYP3A4 | MEP1B | 0.81 | CD.Fibro.TNFSF11.CD24 | MYLK | 1.55 | CD.Prolif/EC.UBE2C.CCNB2 | PCK2 | 0.33 |
| CD.EC.RBP2.CYP3A4 | SLC5A12 | 0.79 | CD.Fibro.TNFSF11.CD24 | MMP2 | 1.52 | CD.Prolif/EC.UBE2C.CCNB2 | TRABD2A | 0.33 |
| CD.EC.RBP2.CYP3A4 | CYP2C18 | 0.77 | CD.Fibro.TNFSF11.CD24 | FBLN1 | 1.40 | CD.Prolif/EC.UBE2C.CCNB2 | GPSM2 | 0.33 |
| CD.EC.RBP2.CYP3A4 | PDZK1 | 0.75 | CD.Fibro.TNFSF11.CD24 | IGFBP5 | 1.33 | CD.Prolif/EC.UBE2C.CCNB2 | RP11- | 0.33 |
| | | | | | | | 532F12.5 | |
| CD.EC.RBP2.CYP3A4 | BTNL8 | 0.74 | CD.Fibro.TNFSF11.CD24 | COL6A1 | 1.33 | CD.Prolif/EC.UBE2C.CCNB2 | FUT3 | 0.33 |
| CD.EC.RBP2.CYP3A4 | RP11- | 0.73 | CD.Fibro.TNFSF11.CD24 | FRZB | 1.31 | CD.Prolif/EC.UBE2C.CCNB2 | RHOBTB3 | 0.33 |
| | 680F8.1 | | | | | | | |
| CD.EC.RBP2.CYP3A4 | COL17A1 | 0.70 | CD.Fibro.TNFSF11.CD24 | TFPI | 1.27 | CD.Prolif/EC.UBE2C.CCNB2 | TKFC | 0.33 |
| CD.EC.RBP2.CYP3A4 | DHDH | 0.65 | CD.Fibro.TNFSF11.CD24 | FHL2 | 1.25 | CD.Prolif/EC.UBE2C.CCNB2 | PPP1R16A | 0.33 |
| CD.EC.RBP2.CYP3A4 | SMLR1 | 0.60 | CD.Fibro.TNFSF11.CD24 | THY1 | 1.25 | CD.Prolif/EC.UBE2C.CCNB2 | F12 | 0.33 |
| CD.EC.RBP2.CYP3A4 | CPO | 0.60 | CD.Fibro.TNFSF11.CD24 | FHL1 | 1.19 | CD.Prolif/EC.UBE2C.CCNB2 | RAD51AP1 | 0.33 |
| CD.EC.RBP2.CYP3A4 | SLC23A1 | 0.46 | CD.Fibro.TNFSF11.CD24 | TNFSF11 | 1.17 | CD.Prolif/EC.UBE2C.CCNB2 | HSD3B7 | 0.32 |
| CD.EC.RBP2.CYP3A4 | SULT1A1 | 1.80 | CD.Fibro.TNFSF11.CD24 | EMILIN1 | 1.16 | CD.Prolif/EC.UBE2C.CCNB2 | GPRC5A | 0.32 |
| CD.EC.RBP2.CYP3A4 | MUC13 | 1.63 | CD.Fibro.TNFSF11.CD24 | MFGE8 | 1.14 | CD.Prolif/EC.UBE2C.CCNB2 | ST6GALNAC1 | 0.32 |
| CD.EC.RBP2.CYP3A4 | PPP1R14D | 1.14 | CD.Fibro.TNFSF11.CD24 | BMP5 | 1.10 | CD.Prolif/EC.UBE2C.CCNB2 | SGOL1 | 0.32 |
| CD.EC.RBP2.CYP3A4 | CIDEB | 1.13 | CD.Fibro.TNFSF11.CD24 | BGN | 1.10 | CD.Prolif/EC.UBE2C.CCNB2 | PRELID2 | 0.32 |
| CD.EC.RBP2.CYP3A4 | KHK | 1.85 | CD.Fibro.TNFSF11.CD24 | CP | 1.10 | CD.Prolif/EC.UBE2C.CCNB2 | GALNT3 | 0.32 |
| CD.EC.RBP2.CYP3A4 | MYH14 | 1.51 | CD.Fibro.TNFSF11.CD24 | PDLIM3 | 0.99 | CD.Prolif/EC.UBE2C.CCNB2 | GPA33 | 0.32 |
| CD.EC.RBP2.CYP3A4 | DPEP1 | 2.19 | CD.Fibro.TNFSF11.CD24 | IL34 | 0.97 | CD.Prolif/EC.UBE2C.CCNB2 | ESRP1 | 0.32 |
| CD.EC.RBP2.CYP3A4 | PRSS3 | 1.48 | CD.Fibro.TNFSF11.CD24 | FAM216B | 0.92 | CD.Prolif/EC.UBE2C.CCNB2 | MRPL17 | 0.32 |
| CD.EC.RBP2.CYP3A4 | PCK1 | 1.68 | CD.Fibro.TNFSF11.CD24 | ALDH1A3 | 0.88 | CD.Prolif/EC.UBE2C.CCNB2 | ESRRA | 0.31 |
| CD.EC.RBP2.CYP3A4 | ZG16 | 1.17 | CD.Fibro.TNFSF11.CD24 | SULF1 | 0.87 | CD.Prolif/EC.UBE2C.CCNB2 | DEGS2 | 0.31 |
| CD.EC.RBP2.CYP3A4 | APOA4 | 2.10 | CD.Fibro.TNFSF11.CD24 | LSAMP | 0.87 | CD.Prolif/EC.UBE2C.CCNB2 | MECOM | 0.31 |
| CD.EC.RBP2.CYP3A4 | TMEM253 | 0.92 | CD.Fibro.TNFSF11.CD24 | ISLR | 0.85 | CD.Prolif/EC.UBE2C.CCNB2 | LRRCC1 | 0.31 |
| CD.EC.RBP2.CYP3A4 | CIDEC | 0.56 | CD.Fibro.TNFSF11.CD24 | C16orf89 | 0.76 | CD.Prolif/EC.UBE2C.CCNB2 | NQO1 | 0.31 |
| CD.EC.RBP2.CYP3A4 | MISP | 1.91 | CD.Fibro.TNFSF11.CD24 | PTGES | 0.75 | CD.Prolif/EC.UBE2C.CCNB2 | KIAA1324 | 0.31 |
| CD.EC.RBP2.CYP3A4 | S100A14 | 1.86 | CD.Fibro.TNFSF11.CD24 | TCF21 | 1.02 | CD.Prolif/EC.UBE2C.CCNB2 | C10orf99 | 0.31 |
| CD.EC.RBP2.CYP3A4 | C19orf33 | 1.96 | CD.Fibro.TNFSF11.CD24 | PTGDS | 1.91 | CD.Prolif/EC.UBE2C.CCNB2 | SLC44A3 | 0.31 |
| CD.EC.RPS15.RPL10 | REG1A | 2.75 | CD.Fibro.TNFSF11.CD24 | CFH | 1.27 | CD.Prolif/EC.UBE2C.CCNB2 | HOXB9 | 0.31 |
| CD.EC.RPS15.RPL10 | KRT18 | 2.36 | CD.Fibro.TNFSF11.CD24 | SERPINE2 | 1.18 | CD.Prolif/EC.UBE2C.CCNB2 | ABO | 0.31 |
| CD.EC.RPS15.RPL10 | KRT8 | 2.29 | CD.Fibro.TNFSF11.CD24 | EFEMP1 | 0.93 | CD.Prolif/EC.UBE2C.CCNB2 | SIGMAR1 | 0.30 |
| CD.EC.RPS15.RPL10 | REG1B | 2.24 | CD.Fibro.TNFSF11.CD24 | CCL2 | 1.75 | CD.Prolif/EC.UBE2C.CCNB2 | URAD | 0.30 |
| CD.EC.RPS15.RPL10 | MT1G | 2.13 | CD.Fibro.TNFSF11.CD24 | COL6A3 | 1.10 | CD.Prolif/EC.UBE2C.CCNB2 | SHROOM3 | 0.30 |
| CD.EC.RPS15.RPL10 | TSPAN8 | 2.08 | CD.Fibro.TNFSF11.CD24 | PRKCDBP | 1.00 | CD.Prolif/EC.UBE2C.CCNB2 | OSR2 | 0.30 |
| CD.EC.RPS15.RPL10 | SPINK1 | 2.03 | CD.Fibro.TNFSF11.CD24 | CXCL13 | 2.28 | CD.Prolif/EC.UBE2C.CCNB2 | ECT2 | 0.30 |
| CD.EC.RPS15.RPL10 | PHGR1 | 1.81 | CD.Fibro.WFDC1.PTN | PTN | 2.06 | CD.Prolif/EC.UBE2C.CCNB2 | MACROD1 | 0.30 |
| CD.EC.RPS15.RPL10 | PLA2G2A | 1.67 | CD.Fibro.WFDC1.PTN | CYGB | 1.88 | CD.Prolif/EC.UBE2C.CCNB2 | ABHD17C | 0.30 |
| CD.EC.SLC28A2.GSTA2 | GSTA2 | 1.68 | CD.Fibro.WFDC1.PTN | HAPLN1 | 1.82 | CD.Prolif/EC.UBE2C.CCNB2 | EPHX2 | 0.30 |
| CD.EC.SLC28A2.GSTA2 | SLC7A9 | 1.25 | CD.Fibro.WFDC1.PTN | ABCA8 | 1.81 | CD.Prolif/EC.UBE2C.CCNB2 | HOXA9 | 0.30 |
| CD.EC.SLC28A2.GSTA2 | MS4A8 | 1.12 | CD.Fibro.WFDC1.PTN | WFDC1 | 1.76 | CD.Prolif/EC.UBE2C.CCNB2 | KIF11 | 0.30 |
| CD.EC.SLC28A2.GSTA2 | SLC28A2 | 1.06 | CD.Fibro.WFDC1.PTN | EDIL3 | 1.63 | CD.Prolif/EC.UBE2C.CCNB2 | FAAH | 0.29 |
| CD.EC.SLC28A2.GSTA2 | RP3-417L20.4 | 0.87 | CD.Fibro.WFDC1.PTN | TRIL | 1.22 | CD.Prolif/EC.UBE2C.CCNB2 | TMEM171 | 0.29 |
| CD.EC.SLC28A2.GSTA2 | SLC6A20 | 0.73 | CD.Fibro.WFDC1.PTN | COL6A5 | 1.16 | CD.Prolif/EC.UBE2C.CCNB2 | DEPDC1 | 0.29 |
| CD.EC.SLC28A2.GSTA2 | AC005550.3 | 0.61 | CD.Fibro.WFDC1.PTN | HHIP | 0.78 | CD.Prolif/EC.UBE2C.CCNB2 | MIS18A | 0.29 |
| CD.EC.SLC28A2.GSTA2 | COL17A1 | 0.60 | CD.Fibro.WFDC1.PTN | BMP5 | 1.57 | CD.Prolif/EC.UBE2C.CCNB2 | UACA | 0.29 |
| CD.EC.SLC28A2.GSTA2 | RP11- | 0.51 | CD.Fibro.WFDC1.PTN | SLITRK6 | 1.21 | CD.Prolif/EC.UBE2C.CCNB2 | GNG12 | 0.29 |
| | 680F8.1 | | | | | | | |
| CD.EC.SLC28A2.GSTA2 | TMIGD1 | 0.69 | CD.Fibro.WFDC1.PTN | FBLN1 | 2.10 | CD.Prolif/EC.UBE2C.CCNB2 | VSIG10 | 0.29 |
| CD.EC.SLC28A2.GSTA2 | ABCG2 | 0.68 | CD.Fibro.WFDC1.PTN | EMILIN1 | 1.66 | CD.Prolif/EC.UBE2C.CCNB2 | KCNQ1 | 0.29 |
| CD.EC.SMOC2.ASCL2 | TSPAN8 | 2.70 | CD.Fibro.WFDC1.PTN | MFAP4 | 2.64 | CD.Prolif/EC.UBE2C.CCNB2 | AGMAT | 0.28 |
| CD.EC.SMOC2.ASCL2 | GPX2 | 2.08 | CD.Fibro/fDC.FDCSP.CXCL13 | FDCSP | 6.03 | CD.Prolif/EC.UBE2C.CCNB2 | GSTO2 | 0.28 |
| CD.EC.SMOC2.ASCL2 | CLDN3 | 1.88 | CD.Fibro/fDC.FDCSP.CXCL13 | CXCL13 | 4.61 | CD.Prolif/EC.UBE2C.CCNB2 | DDR1 | 0.28 |
| CD.EC.SMOC2.ASCL2 | UGT2B17 | 1.48 | CD.Fibro/fDC.FDCSP.CXCL13 | SERPINE2 | 2.97 | CD.Prolif/EC.UBE2C.CCNB2 | PRRG4 | 0.28 |
| CD.EC.SMOC2.ASCL2 | SPINK1 | 2.03 | CD.Fibro/fDC.FDCSP.CXCL13 | LTF | 2.76 | CD.Prolif/EC.UBE2C.CCNB2 | ARHGEF35 | 0.28 |
| CD.EC.SMOC2.ASCL2 | EDN1 | 1.38 | CD.Fibro/fDC.FDCSP.CXCL13 | FCAMR | 2.09 | CD.Prolif/EC.UBE2C.CCNB2 | PDSS1 | 0.28 |
| CD.EC.SMOC2.ASCL2 | OLFM4 | 2.74 | CD.Fibro/fDC.FDCSP.CXCL13 | NPPC | 1.67 | CD.Prolif/EC.UBE2C.CCNB2 | PDE4C | 0.28 |
| CD.EC.TCL1A.IFI6 | PIGR | 3.34 | CD.Fibro/fDC.FDCSP.CXCL13 | ANKRD29 | 1.55 | CD.Prolif/EC.UBE2C.CCNB2 | ATP2C2 | 0.28 |
| CD.EC.TCL1A.IFI6 | OLFM4 | 2.16 | CD.Fibro/fDC.FDCSP.CXCL13 | NPB | 1.50 | CD.Prolif/EC.UBE2C.CCNB2 | GATA6 | 0.27 |
| CD.EC.TMPRSS15.FAM8A1 | TMPRSS15 | 0.99 | CD.Fibro/fDC.FDCSP.CXCL13 | TMEM163 | 1.43 | CD.Prolif/EC.UBE2C.CCNB2 | RNF43 | 0.27 |
| CD.EC.TMPRSS15.FAM8A1 | CYP2C18 | 0.95 | CD.Fibro/fDC.FDCSP.CXCL13 | SERPINA5 | 1.40 | CD.Prolif/EC.UBE2C.CCNB2 | CHMP4C | 0.27 |
| CD.EC.UBD.IFITM1 | DLGAP1-AS5 | 0.30 | CD.Fibro/fDC.FDCSP.CXCL13 | S100A1 | 1.38 | CD.Prolif/EC.UBE2C.CCNB2 | MAP7 | 0.27 |
| CD.EC.UBD.IFITM1 | UBD | 0.72 | CD.Fibro/fDC.FDCSP.CXCL13 | RGS9 | 1.17 | CD.Prolif/EC.UBE2C.CCNB2 | SNX7 | 0.27 |
| CD.Endth.IFI6.ISG15 | IFI27 | 3.16 | CD.Fibro/fDC.FDCSP.CXCL13 | PRRX1 | 1.15 | CD.Prolif/EC.UBE2C.CCNB2 | SHMT1 | 0.27 |
| CD.Endth.IFI6.ISG15 | ACKR1 | 3.12 | CD.Fibro/fDC.FDCSP.CXCL13 | LSAMP | 1.11 | CD.Prolif/EC.UBE2C.CCNB2 | AKR1B10 | 0.26 |
| CD.Endth.IFI6.ISG15 | IGFBP7 | 2.94 | CD.Fibro/fDC.FDCSP.CXCL13 | SCARA3 | 0.95 | CD.Prolif/EC.UBE2C.CCNB2 | H2AFY2 | 0.26 |
| CD.Endth.IFI6.ISG15 | PLVAP | 2.93 | CD.Fibro/fDC.FDCSP.CXCL13 | CLDN16 | 0.94 | CD.Prolif/EC.UBE2C.CCNB2 | MT1A | 0.26 |
| CD.Endth.IFI6.ISG15 | SPARCL1 | 2.84 | CD.Fibro/fDC.FDCSP.CXCL13 | PCOLCE2 | 0.91 | CD.Prolif/EC.UBE2C.CCNB2 | GALE | 0.26 |
| CD.Endth.IFI6.ISG15 | IFI6 | 2.72 | CD.Fibro/fDC.FDCSP.CXCL13 | RBPMS-AS1 | 0.81 | CD.Prolif/EC.UBE2C.CCNB2 | HOXA10 | 0.25 |
| CD.Endth.IFI6.ISG15 | IFITM3 | 2.63 | CD.Fibro/fDC.FDCSP.CXCL13 | PRELP | 0.80 | CD.Prolif/EC.UBE2C.CCNB2 | MYO5B | 0.25 |
| CD.Endth.IFI6.ISG15 | CD320 | 2.46 | CD.Fibro/fDC.FDCSP.CXCL13 | RP11- | 0.75 | CD.Prolif/EC.UBE2C.CCNB2 | CDCA2 | 0.25 |
| | | | | 283G6.4 | | | | |
| CD.Endth.IFI6.ISG15 | CLDN5 | 2.40 | CD.Fibro/fDC.FDCSP.CXCL13 | S1PR3 | 0.66 | CD.Prolif/EC.UBE2C.CCNB2 | SLC37A4 | 0.28 |
| CD.Endth.IFI6.ISG15 | RAMP2 | 2.34 | CD.Fibro/fDC.FDCSP.CXCL13 | SLC1A2 | 0.58 | CD.Prolif/EC.UBE2C.CCNB2 | MGST3 | 0.91 |
| CD.Endth.IFI6.ISG15 | SLC9A3R2 | 2.29 | CD.Fibro/fDC.FDCSP.CXCL13 | TF | 0.50 | CD.Prolif/EC.UBE2C.CCNB2 | ANG | 0.31 |
| CD.Endth.IFI6.ISG15 | TM4SF1 | 2.29 | CD.Fibro/fDC.FDCSP.CXCL13 | PLA2G5 | 0.44 | CD.Prolif/EC.UBE2C.CCNB2 | TMEM261 | 0.52 |
| CD.Endth.IFI6.ISG15 | CRIP2 | 2.25 | CD.Fibro/fDC.FDCSP.CXCL13 | OVOS2 | 1.12 | CD.Prolif/EC.UBE2C.CCNB2 | SGOL2 | 0.32 |
| CD.Endth.IFI6.ISG15 | RAMP3 | 2.23 | CD.Fibro/fDC.FDCSP.CXCL13 | VIT | 0.69 | CD.Prolif/EC.UBE2C.CCNB2 | TUBB4B | 1.22 |
| CD.Endth.IFI6.ISG15 | IFI44L | 2.15 | CD.Fibro/fDC.FDCSP.CXCL13 | AC016995.3 | 0.45 | CD.Prolif/EC.UBE2C.CCNB2 | FARSB | 0.30 |
| CD.Endth.IFI6.ISG15 | CAV1 | 2.15 | CD.Fibro/fDC.FDCSP.CXCL13 | LRTM1 | 0.39 | CD.Prolif/EC.UBE2C.CCNB2 | ADTRP | 0.25 |
| CD.Endth.IFI6.ISG15 | IFIT1 | 2.09 | CD.Glial.COL28A1.SPP1 | SPP1 | 3.63 | CD.Prolif/EC.UBE2C.CCNB2 | CRB3 | 0.27 |
| CD.Endth.IFI6.ISG15 | GNG11 | 2.07 | CD.Glial.COL28A1.SPP1 | CRYAB | 3.50 | CD.Prolif/EC.UBE2C.CCNB2 | TSTA3 | 0.39 |
| CD.Endth.IFI6.ISG15 | PRSS23 | 2.02 | CD.Glial.COL28A1.SPP1 | SCN7A | 3.38 | CD.Prolif/EC.UBE2C.CCNB2 | NEK2 | 0.26 |
| CD.Endth.IFI6.ISG15 | MGP | 1.93 | CD.Glial.COL28A1.SPP1 | PLP1 | 3.24 | CD.Prolif/EC.UBE2C.CCNB2 | VDR | 0.30 |
| CD.Endth.IFI6.ISG15 | ENG | 1.92 | CD.Glial.COL28A1.SPP1 | S100B | 2.85 | CD.Prolif/EC.UBE2C.CCNB2 | STRA13 | 0.56 |
| CD.Endth.IFI6.ISG15 | IGFBP4 | 1.92 | CD.Glial.COL28A1.SPP1 | CDH19 | 2.71 | CD.Prolif/EC.UBE2C.CCNB2 | OLA1 | 0.45 |
| CD.Endth.IFI6.ISG15 | CLEC14A | 1.81 | CD.Glial.COL28A1.SPP1 | LG14 | 2.62 | CD.Prolif/EC.UBE2C.CCNB2 | ZDHHC3 | 0.30 |
| CD.Endth.IFI6.ISG15 | NPDC1 | 1.68 | CD.Glial.COL28A1.SPP1 | NRXN1 | 2.48 | CD.Prolif/EC.UBE2C.CCNB2 | HSPD1 | 1.11 |
| CD.Endth.IFI6.ISG15 | IFIT3 | 1.65 | CD.Glial.COL28A1.SPP1 | MYOT | 2.36 | CD.Prolif/EC.UBE2C.CCNB2 | AURKA | 0.42 |
| CD.Endth.IFI6.ISG15 | VWA1 | 1.65 | CD.Glial.COL28A1.SPP1 | CAB39L | 2.28 | CD.Prolif/EC.UBE2C.CCNB2 | PBX1 | 0.27 |
| CD.Endth.IFI6.ISG15 | ESAM | 1.64 | CD.Glial.COL28A1.SPP1 | COL28A1 | 2.26 | CD.Prolif/EC.UBE2C.CCNB2 | TSPAN3 | 0.48 |
| CD.Endth.IFI6.ISG15 | SPARC | 1.63 | CD.Glial.COL28A1.SPP1 | XKR4 | 1.91 | CD.Prolif/EC.UBE2C.CCNB2 | KNOP1 | 0.31 |
| CD.Endth.IFI6.ISG15 | BCAM | 1.63 | CD.Glial.COL28A1.SPP1 | GFRA3 | 1.90 | CD.Prolif/EC.UBE2C.CCNB2 | RBM47 | 0.41 |
| CD.Endth.IFI6.ISG15 | TSPAN7 | 1.59 | CD.Glial.COL28A1.SPP1 | SLITRK6 | 1.80 | CD.Prolif/EC.UBE2C.CCNB2 | FLNB | 0.29 |
| CD.Endth.IFI6.ISG15 | EGFL7 | 1.58 | CD.Glial.COL28A1.SPP1 | COL21A1 | 1.77 | CD.Prolif/EC.UBE2C.CCNB2 | AGPAT2 | 0.40 |
| CD.Endth.IFI6.ISG15 | EMCN | 1.58 | CD.Glial.COL28A1.SPP1 | NLGN4X | 1.77 | CD.Prolif/EC.UBE2C.CCNB2 | CENPE | 0.35 |
| CD.Endth.IFI6.ISG15 | MADCAM1 | 1.57 | CD.Glial.COL28A1.SPP1 | PTPRZ1 | 1.56 | CD.Prolif/EC.UBE2C.CCNB2 | ESCO2 | 0.28 |
| CD.Endth.IFI6.ISG15 | RBP5 | 1.56 | CD.Glial.COL28A1.SPP1 | MPZ | 1.55 | CD.Prolif/EC.UBE2C.CCNB2 | AK3 | 0.44 |
| CD.Endth.IFI6.ISG15 | PTRF | 1.53 | CD.Glial.COL28A1.SPP1 | TSPAN11 | 1.43 | CD.Prolif/EC.UBE2C.CCNB2 | HOOK1 | 0.29 |
| CD.Endth.IFI6.ISG15 | TMEM88 | 1.52 | CD.Glial.COL28A1.SPP1 | PCSK2 | 1.40 | CD.Prolif/EC.UBE2C.CCNB2 | TSPO | 0.73 |
| CD.Endth.IFI6.ISG15 | VWF | 1.50 | CD.Glial.COL28A1.SPP1 | HAND2 | 1.39 | CD.Prolif/EC.UBE2C.CCNB2 | NDUFAB1 | 0.78 |
| CD.Endth.IFI6.ISG15 | FAM167B | 1.49 | CD.Glial.COL28A1.SPP1 | WDR86 | 1.39 | CD.Prolif/EC.UBE2C.CCNB2 | S100A10 | 1.10 |
| CD.Endth.IFI6.ISG15 | JAM2 | 1.42 | CD.Glial.COL28A1.SPP1 | CADM3 | 1.39 | CD.Prolif/EC.UBE2C.CCNB2 | EBPL | 0.41 |
| CD.Endth.IFI6.ISG15 | ECSCR-1 | 1.42 | CD.Glial.COL28A1.SPP1 | LRRC4C | 1.32 | CD.Prolif/EC.UBE2C.CCNB2 | MRPS12 | 0.40 |
| CD.Endth.IFI6.ISG15 | NOSTRIN | 1.40 | CD.Glial.COL28A1.SPP1 | CADM2 | 1.30 | CD.Prolif/EC.UBE2C.CCNB2 | SMAGP | 0.27 |
| CD.Endth.IFI6.ISG15 | TINAGL1 | 1.40 | CD.Glial.COL28A1.SPP1 | NTM | 1.29 | CD.Prolif/EC.UBE2C.CCNB2 | JUF | 0.31 |
| CD.Endth.IFI6.ISG15 | TM4SF18 | 1.40 | CD.Glial.COL28A1.SPP1 | NRXN3 | 1.19 | CD.Prolif/EC.UBE2C.CCNB2 | NDUFV1 | 0.55 |
| CD.Endth.IFI6.ISG15 | FLT1 | 1.39 | CD.Glial.COL28A1.SPP1 | NCAM2 | 1.04 | CD.Prolif/EC.UBE2C.CCNB2 | ARPC1A | 0.45 |
| CD.Endth.IFI6.ISG15 | ADGRL4 | 1.31 | CD.Glial.COL28A1.SPP1 | FOXD3-AS1 | 1.04 | CD.Prolif/EC.UBE2C.CCNB2 | KIF9 | 0.27 |
| CD.Endth.IFI6.ISG15 | ARHGAP29 | 1.30 | CD.Glial.COL28A1.SPP1 | LRRTM1 | 1.03 | CD.Prolif/EC.UBE2C.CCNB2 | FNBP1L | 0.26 |
| CD.Endth.IFI6.ISG15 | SNCG | 1.30 | CD.Glial.COL28A1.SPP1 | MGAT4C | 0.97 | CD.Prolif/EC.UBE2C.CCNB2 | STOML2 | 0.55 |
| CD.Endth.IFI6.ISG15 | CD34 | 1.21 | CD.Glial.COL28A1.SPP1 | L1CAM | 0.97 | CD.Prolif/EC.UBE2C.CCNB2 | MRPL11 | 0.49 |
| CD.Endth.IFI6.ISG15 | PCAT19 | 1.15 | CD.Glial.COL28A1.SPP1 | SOX2 | 0.96 | CD.Prolif/EC.UBE2C.CCNB2 | PHB2 | 0.56 |
| CD.Endth.IFI6.ISG15 | LCN6 | 1.09 | CD.Glial.COL28A1.SPP1 | NKAIN3 | 0.90 | CD.Prolif/EC.UBE2C.CCNB2 | DKC1 | 0.40 |
| CD.Endth.IFI6.ISG15 | CYYR1 | 1.08 | CD.Glial.COL28A1.SPP1 | CHST9 | 0.62 | CD.Prolif/EC.UBE2C.CCNB2 | NR1H4 | 0.28 |
| CD.Endth.IFI6.ISG15 | KANK3 | 0.92 | CD.Glial.COL28A1.SPP1 | RP11- | 0.56 | CD.Prolif/EC.UBE2C.CCNB2 | GNPNAT1 | 0.31 |
| | | | | 308N19.1 | | | | |
| CD.Endth.IFI6.ISG15 | CXorf36 | 0.92 | CD.Glial.COL28A1.SPP1 | CYTL1 | 1.28 | CD.Prolif/EC.UBE2C.CCNB2 | GAPDH | 1.13 |
| CD.Endth.IFI6.ISG15 | PCDH17 | 0.94 | CD.Glial.COL28A1.SPP1 | CMTM5 | 0.80 | CD.Prolif/EC.UBE2C.CCNB2 | FAM162A | 0.48 |
| CD.Endth.IFI6.ISG15 | RSAD2 | 1.00 | CD.Glial.COL28A1.SPP1 | SEMA3B | 2.36 | CD.Prolif/EC.UBE2C.CCNB2 | ETFB | 0.75 |
| CD.Endth.IFI6.ISG15 | IL3RA | 1.23 | CD.Glial.COL28A1.SPP1 | LINC01186 | 0.54 | CD.Prolif/EC.UBE2C.CCNB2 | TMPRSS2 | 0.26 |
| CD.Endth.IFI6.ISG15 | HYAL2 | 1.44 | CD.Glial.COL28A1.SPP1 | IGSF11 | 0.54 | CD.Prolif/EC.UBE2C.CCNB2 | NUDT8 | 0.29 |
| CD.Endth.IFI6.ISG15 | SLCO2A1 | 0.96 | CD.Glial.COL28A1.SPP1 | SERPINA5 | 0.93 | CD.Prolif/EC.UBE2C.CCNB2 | FKBP4 | 0.38 |
| CD.Endth.IFI6.ISG15 | FAM110D | 0.83 | CD.Glial.S100B.PLP1 | CRYAB | 3.68 | CD.Prolif/EC.UBE2C.CCNB2 | DECR1 | 0.52 |
| CD.Endth.IFI6.ISG15 | MMRN2 | 0.90 | CD.Glial.S100B.PLP1 | S100B | 3.38 | CD.Prolif/EC.UBE2C.CCNB2 | BLVRB | 0.43 |
| CD.Endth.IFI6.ISG15 | ID1 | 2.09 | CD.Glial.S100B.PLP1 | PLP1 | 2.92 | CD.Prolif/EC.UBE2C.CCNB2 | COQ2 | 0.29 |
| CD.Endth.MT-CO3.MT-CO2 | VWF | 2.83 | CD.Glial.S100B.PLP1 | LGI4 | 2.62 | CD.Prolif/EC.UBE2C.CCNB2 | CCT5 | 0.50 |
| CD.Endth.MT-CO3.MT-CO2 | HSPG2 | 2.34 | CD.Glial.S100B.PLP1 | CDH19 | 2.06 | CD.Prolif/EC.UBE2C.CCNB2 | LMO7 | 0.28 |
| CD.Endth.MT-CO3.MT-CO2 | PLVAP | 2.33 | CD.Glial.S100B.PLP1 | GFRA3 | 1.68 | CD.Prolif/EC.UBE2C.CCNB2 | GALNT1 | 0.30 |
| CD.Endth.MT-CO3.MT-CO2 | CLDN5 | 2.33 | CD.Glial.S100B.PLP1 | SPP1 | 2.61 | CD.Prolif/EC.UBE2C.CCNB2 | EIF6 | 0.56 |
| CD.Endth.MT-CO3.MT-CO2 | SPARCL1 | 2.31 | CD.Glial.S100B.SPP1 | CRYAB | 3.98 | CD.Prolif/EC.UBE2C.CCNB2 | ATP5G3 | 1.13 |
| CD.Endth.MT-CO3.MT-CO2 | ACKR1 | 2.17 | CD.Glial.S100B.SPP1 | SPP1 | 3.43 | CD.Prolif/EC.UBE2C.CCNB2 | KRT20 | 0.45 |
| CD.Endth.MT-CO3.MT-CO2 | EGFL7 | 1.92 | CD.Glial.S100B.SPP1 | S100B | 3.39 | CD.Prolif/EC.UBE2C.CCNB2 | SLIRP | 0.79 |
| CD.Endth.MT-CO3.MT-CO2 | RAMP3 | 1.69 | CD.Glial.S100B.SPP1 | FXYD1 | 3.26 | CD.Prolif/EC.UBE2C.CCNB2 | NDUFA10 | 0.42 |
| CD.Endth.MT-CO3.MT-CO2 | TLL1 | 1.25 | CD.Glial.S100B.SPP1 | PLP1 | 3.17 | CD.Prolif/EC.UBE2C.CCNB2 | ALDH1B1 | 0.29 |
| CD.Endth/Art.SEMA3G.SSUH2 | TM4SF1 | 3.11 | CD.Glial.S100B.SPP1 | LGI4 | 2.94 | CD.Prolif/EC.UBE2C.CCNB2 | ADD3 | 0.44 |
| CD.Endth/Art.SEMA3G.SSUH2 | RAMP2 | 3.01 | CD.Glial.S100B.SPP1 | SCN7A | 2.89 | CD.Prolif/EC.UBE2C.CCNB2 | CD151 | 0.42 |
| CD.Endth/Art.SEMA3G.SSUH2 | SLC9A3R2 | 2.96 | CD.Glial.S100B.SPP1 | NRXN1 | 2.66 | CD.Prolif/EC.UBE2C.CCNB2 | PPIH | 0.35 |
| CD.Endth/Art.SEMA3G.SSUH2 | CLDN5 | 2.83 | CD.Glial.S100B.SPP1 | MATN2 | 2.62 | CD.Prolif/EC.UBE2C.CCNB2 | CANT1 | 0.28 |
| CD.Endth/Art.SEMA3G.SSUH2 | CAV1 | 2.68 | CD.Glial.S100B.SPP1 | MYOT | 2.49 | CD.Prolif/EC.UBE2C.CCNB2 | TTK | 0.28 |
| CD.Endth/Art.SEMA3G.SSUH2 | CRIP2 | 2.53 | CD.Glial.S100B.SPP1 | SEMA3B | 2.39 | CD.Prolif/EC.UBE2C.CCNB2 | C1QBP | 0.77 |
| CD.Endth/Art.SEMA3G.SSUH2 | CXCL12 | 2.43 | CD.Glial.S100B.SPP1 | MPZ | 2.31 | CD.Prolif/EC.UBE2C.CCNB2 | TMEM176A | 0.30 |
| CD.Endth/Art.SEMA3G.SSUH2 | PLVAP | 2.31 | CD.Glial.S100B.SPP1 | CDH19 | 2.25 | CD.Prolif/EC.UBE2C.CCNB2 | PRR11 | 0.27 |
| CD.Endth/Art.SEMA3G.SSUH2 | GNG11 | 2.27 | CD.Glial.S100B.SPP1 | GFRA3 | 2.23 | CD.Prolif/EC.UBE2C.CCNB2 | NR2F6 | 0.26 |
| CD.Endth/Art.SEMA3G.SSUH2 | TIMP3 | 2.21 | CD.Glial.S100B.SPP1 | RCAN1 | 2.18 | CD.Prolif/EC.UBE2C.CCNB2 | CXCL3 | 0.49 |
| CD.Endth/Art.SEMA3G.SSUH2 | MGP | 2.18 | CD.Glial.S100B.SPP1 | ANK3 | 2.08 | CD.Prolif/EC.UBE2C.CCNB2 | ABCC3 | 0.28 |
| CD.Endth/Art.SEMA3G.SSUH2 | EPAS1 | 2.17 | CD.Glial.S100B.SPP1 | DKK3 | 2.06 | CD.Prolif/EC.UBE2C.CCNB2 | CENPK | 0.28 |
| CD.Endth/Art.SEMA3G.SSUH2 | RNASE1 | 2.17 | CD.Glial.S100B.SPP1 | SORBS2 | 1.96 | CD.Prolif/EC.UBE2C.CCNB2 | UQCC3 | 0.38 |
| CD.Endth/Art.SEMA3G.SSUH2 | FCN3 | 2.08 | CD.Glial.S100B.SPP1 | FBLN2 | 1.91 | CD.Prolif/EC.UBE2C.CCNB2 | SMARCC1 | 0.39 |
| CD.Endth/Art.SEMA3G.SSUH2 | C10orf10 | 2.05 | CD.Glial.S100B.SPP1 | NTM | 1.83 | CD.Prolif/EC.UBE2C.CCNB2 | AK2 | 0.42 |
| CD.Endth/Art.SEMA3G.SSUH2 | PTRF | 2.02 | CD.Glial.S100B.SPP1 | NRN1 | 1.80 | CD.Prolif/EC.UBE2C.CCNB2 | ANXA3 | 0.27 |
| CD.Endth/Art.SEMA3G.SSUH2 | NPDC1 | 2.01 | CD.Glial.S100B.SPP1 | CAB39L | 1.78 | CD.Prolif/EC.UBE2C.CCNB2 | PSMB5 | 0.57 |
| CD.Endth/Art.SEMA3G.SSUH2 | JAM2 | 1.92 | CD.Glial.S100B.SPP1 | ABI3BP | 1.76 | CD.Prolif/EC.UBE2C.CCNB2 | H2AFJ | 0.47 |
| CD.Endth/Art.SEMA3G.SSUH2 | PODXL | 1.86 | CD.Glial.S100B.SPP1 | XKR4 | 1.72 | CD.Prolif/EC.UBE2C.CCNB2 | FCGRT | 0.53 |
| CD.Endth/Art.SEMA3G.SSUH2 | EGFL7 | 1.82 | CD.Glial.S100B.SPP1 | TSPAN11 | 1.63 | CD.Prolif/Secretory.REG1B.MGST1 | REG1B | 3.54 |
| CD.Endth/Art.SEMA3G.SSUH2 | HYAL2 | 1.81 | CD.Glial.S100B.SPP1 | CADM2 | 1.61 | CD.Prolif/Secretory.REG1B.MGST1 | OLFM4 | 2.83 |
| CD.Endth/Art.SEMA3G.SSUH2 | CLEC14A | 1.78 | CD.Glial.S100B.SPP1 | PCDH9 | 1.57 | CD.Prolif/Secretory.REG1B.MGST1 | PLA2G2A | 2.43 |
| CD.Endth/Art.SEMA3G.SSUH2 | EMCN | 1.76 | CD.Glial.S100B.SPP1 | PRIMA1 | 1.52 | CD.Prolif/Secretory.REG1B.MGST1 | PIGR | 2.41 |
| CD.Endth/Art.SEMA3G.SSUH2 | ESAM | 1.75 | CD.Glial.S100B.SPP1 | L1CAM | 1.50 | CD.Prolif/Secretory.REG1B.MGST1 | TSPAN8 | 2.37 |
| CD.Endth/Art.SEMA3G.SSUH2 | SOX17 | 1.72 | CD.Glial.S100B.SPP1 | MAP1B | 1.48 | CD.Prolif/Secretory.REG1B.MGST1 | DMBT1 | 2.13 |
| CD.Endth/Art.SEMA3G.SSUH2 | BCAM | 1.72 | CD.Glial.S100B.SPP1 | FEZ1 | 1.45 | CD.Prolif/Secretory.REG1B.MGST1 | EPCAM | 2.11 |
| CD.Endth/Art.SEMA3G.SSUH2 | CD34 | 1.69 | CD.Glial.S100B.SPP1 | LRRTM1 | 1.40 | CD.Prolif/Secretory.REG1B.MGST1 | KRT19 | 2.07 |
| CD.Endth/Art.SEMA3G.SSUH2 | ECSCR-1 | 1.69 | CD.Glial.S100B.SPP1 | CYTL1 | 1.40 | CD.Prolif/Secretory.REG1B.MGST1 | SPINK1 | 2.02 |
| CD.Endth/Art.SEMA3G.SSUH2 | ADGRL4 | 1.64 | CD.Glial.S100B.SPP1 | NLGN4X | 1.38 | CD.Prolif/Secretory.REG1B.MGST1 | MGST1 | 1.98 |
| CD.Endth/Art.SEMA3G.SSUH2 | PALMD | 1.60 | CD.Glial.S100B.SPP1 | TIMP4 | 1.37 | CD.Prolif/Secretory.REG1B.MGST1 | LGALS4 | 1.88 |
| CD.Endth/Art.SEMA3G.SSUH2 | HEG1 | 1.58 | CD.Glial.S100B.SPP1 | COL8A1 | 1.36 | CD.Prolif/Secretory.REG1B.MGST1 | GPX2 | 1.83 |
| CD.Endth/Art.SEMA3G.SSUH2 | PTPRB | 1.57 | CD.Glial.S100B.SPP1 | CDH2 | 1.35 | CD.Prolif/Secretory.REG1B.MGST1 | UGT2B17 | 1.78 |
| CD.Endth/Art.SEMA3G.SSUH2 | SEMA3G | 1.56 | CD.Glial.S100B.SPP1 | OLFML2A | 1.34 | CD.Prolif/Secretory.REG1B.MGST1 | AGR2 | 1.77 |
| CD.Endth/Art.SEMA3G.SSUH2 | CYYR1 | 1.56 | CD.Glial.S100B.SPP1 | COL28A1 | 1.31 | CD.Prolif/Secretory.REG1B.MGST1 | LCN2 | 1.76 |
| CD.Endth/Art.SEMA3G.SSUH2 | ADGRF5 | 1.51 | CD.Glial.S100B.SPP1 | TTR | 1.22 | CD.Prolif/Secretory.REG1B.MGST1 | AKR1C3 | 1.67 |
| CD.Endth/Art.SEMA3G.SSUH2 | GJA4 | 1.51 | CD.Glial.S100B.SPP1 | NGFR | 1.20 | CD.Prolif/Secretory.REG1B.MGST1 | AGR3 | 1.65 |
| CD.Endth/Art.SEMA3G.SSUH2 | HEY1 | 1.47 | CD.Glial.S100B.SPP1 | COL21A1 | 1.19 | CD.Prolif/Secretory.REG1B.MGST1 | CLDN7 | 1.58 |
| CD.Endth/Art.SEMA3G.SSUH2 | IGF2 | 1.43 | CD.Glial.S100B.SPP1 | PCSK2 | 1.18 | CD.Prolif/Secretory.REG1B.MGST1 | SLC12A2 | 1.39 |
| CD.Endth/Art.SEMA3G.SSUH2 | PCAT19 | 1.39 | CD.Glial.S100B.SPP1 | WDR86 | 1.16 | CD.Prolif/Secretory.REG1B.MGST1 | CCL25 | 1.32 |
| CD.Endth/Art.SEMA3G.SSUH2 | SOX18 | 1.32 | CD.Glial.S100B.SPP1 | ST6GALNAC2 | 1.12 | CD.Prolif/Secretory.REG1B.MGST1 | FABP2 | 1.26 |
| CD.Endth/Art.SEMA3G.SSUH2 | CDH5 | 1.28 | CD.Glial.S100B.SPP1 | PTPRZ1 | 1.11 | CD.Prolif/Secretory.REG1B.MGST1 | KLF5 | 1.24 |
| CD.Endth/Art.SEMA3G.SSUH2 | MMRN2 | 1.27 | CD.Glial.S100B.SPP1 | ART3 | 1.10 | CD.Prolif/Secretory.REG1B.MGST1 | CLDN3 | 1.22 |
| CD.Endth/Art.SEMA3G.SSUH2 | NOTCH4 | 1.26 | CD.Glial.S100B.SPP1 | FOXD3-AS1 | 1.09 | CD.Prolif/Secretory.REG1B.MGST1 | S100A14 | 1.10 |
| CD.Endth/Art.SEMA3G.SSUH2 | AIF1L | 1.26 | CD.Glial.S100B.SPP1 | CADM3 | 1.07 | CD.Prolif/Secretory.REG1B.MGST1 | CPS1 | 1.09 |
| CD.Endth/Art.SEMA3G.SSUH2 | MYCT1 | 1.23 | CD.Glial.S100B.SPP1 | SBSPON | 1.03 | CD.Prolif/Secretory.REG1B.MGST1 | TMEM54 | 1.09 |
| CD.Endth/Art.SEMA3G.SSUH2 | FAM107A | 1.18 | CD.Glial.S100B.SPP1 | ZNF536 | 1.03 | CD.Prolif/Secretory.REG1B.MGST1 | CES2 | 1.08 |
| CD.Endth/Art.SEMA3G.SSUH2 | GJA5 | 1.13 | CD.Glial.S100B.SPP1 | MGAT4C | 1.02 | CD.Prolif/Secretory.REG1B.MGST1 | ADH1C | 1.08 |
| CD.Endth/Art.SEMA3G.SSUH2 | SLC14A1 | 1.13 | CD.Glial.S100B.SPP1 | SOX10 | 1.02 | CD.Prolif/Secretory.REG1B.MGST1 | ALDOB | 1.05 |
| CD.Endth/Art.SEMA3G.SSUH2 | NES | 1.11 | CD.Glial.S100B.SPP1 | SOX2 | 0.98 | CD.Prolif/Secretory.REG1B.MGST1 | RARRES1 | 1.05 |
| CD.Endth/Art.SEMA3G.SSUH2 | PTPRM | 1.10 | CD.Glial.S100B.SPP1 | SYT10 | 0.93 | CD.Prolif/Secretory.REG1B.MGST1 | SMIM22 | 1.03 |
| CD.Endth/Art.SEMA3G.SSUH2 | KDR | 1.06 | CD.Glial.S100B.SPP1 | SORCS1 | 0.89 | CD.Prolif/Secretory.REG1B.MGST1 | KCNJ13 | 1.02 |
| CD.Endth/Art.SEMA3G.SSUH2 | JAG2 | 0.98 | CD.Glial.S100B.SPP1 | CMTM5 | 0.88 | CD.Prolif/Secretory.REG1B.MGST1 | DPEP1 | 0.99 |
| CD.Endth/Art.SEMA3G.SSUH2 | SOX7 | 0.90 | CD.Glial.S100B.SPP1 | ANGPTL7 | 0.84 | CD.Prolif/Secretory.REG1B.MGST1 | FAM3B | 0.96 |
| CD.Endth/Art.SEMA3G.SSUH2 | PI16 | 0.87 | CD.Glial.S100B.SPP1 | HSPA12A | 0.82 | CD.Prolif/Secretory.REG1B.MGST1 | PPP1R1B | 0.92 |
| CD.Endth/Art.SEMA3G.SSUH2 | ALPL | 0.85 | CD.Glial.S100B.SPP1 | RXRG | 0.80 | CD.Prolif/Secretory.REG1B.MGST1 | PRSS3 | 0.90 |
| CD.Endth/Art.SEMA3G.SSUH2 | HSPA12B | 0.76 | CD.Glial.S100B.SPP1 | ST6GALNAC5 | 0.76 | CD.Prolif/Secretory.REG1B.MGST1 | MAOA | 0.87 |
| CD.Endth/Art.SEMA3G.SSUH2 | SLCO2A1 | 1.29 | CD.Glial.S100B.SPP1 | LG11 | 0.70 | CD.Prolif/Secretory.REG1B.MGST1 | CDH17 | 0.84 |
| CD.Endth/Art.SEMA3G.SSUH2 | CD93 | 1.66 | CD.Glial.S100B.SPP1 | GRIA1 | 0.66 | CD.Prolif/Secretory.REG1B.MGST1 | DSG2 | 0.81 |
| CD.Endth/Art.SEMA3G.SSUH2 | TIE1 | 1.12 | CD.Glial.S100B.SPP1 | RP11- | 0.61 | CD.Prolif/Secretory.REG1B.MGST1 | CDX1 | 0.81 |
| | | | | 308N19.1 | | | | |
| CD.Endth/Art.SEMA3G.SSUH2 | SPARCL1 | 2.37 | CD.Glial.S100B.SPP1 | MAB21L1 | 0.78 | CD.Prolif/Secretory.REG1B.MGST1 | KRTCAP3 | 0.78 |
| CD.Endth/Art.SEMA3G.SSUH2 | RAMP3 | 1.74 | CD.Glial.S100B.SPP1 | PMP2 | 0.71 | CD.Prolif/Secretory.REG1B.MGST1 | RAB25 | 0.77 |
| CD.Endth/Art.SEMA3G.SSUH2 | AQP1 | 1.69 | CD.Glial.S100B.SPP1 | SEMA3C | 1.27 | CD.Prolif/Secretory.REG1B.MGST1 | PKP2 | 0.75 |
| CD.Endth/Art.SEMA3G.SSUH2 | SSUH2 | 0.92 | CD.Glial.S100B.SPP1 | SNCA | 1.25 | CD.Prolif/Secretory.REG1B.MGST1 | CHP2 | 0.70 |
| CD.Endth/Art.SEMA3G.SSUH2 | FLT1 | 1.36 | CD.Glial.S100B.SPP1.1 | LGI4 | 2.49 | CD.Prolif/Secretory.REG1B.MGST1 | VIL1 | 0.69 |
| CD.Endth/Art.SEMA3G.SSUH2 | NOV | 0.94 | CD.Glial.S100B.SPP1.1 | SPP1 | 2.48 | CD.Prolif/Secretory.REG1B.MGST1 | GOLM1 | 0.68 |
| CD.Endth/Art.SEMA3G.SSUH2 | PLS3 | 1.49 | CD.Glial.S100B.SPP1.1 | PLP1 | 2.44 | CD.Prolif/Secretory.REG1B.MGST1 | OTC | 0.64 |
| CD.Endth/Art.SEMA3G.SSUH2 | STC1 | 1.00 | CD.Glial.S100B.SPP1.1 | MYOT | 1.96 | CD.Prolif/Secretory.REG1B.MGST1 | SI | 0.64 |
| CD.Endth/Art.SEMA3G.SSUH2 | LDB2 | 1.30 | CD.Glial.S100B.SPP1.1 | NRXN1 | 1.78 | CD.Prolif/Secretory.REG1B.MGST1 | CCL15 | 0.63 |
| CD.Endth/Art.SEMA3G.SSUH2 | TMEM100 | 1.48 | CD.Glial.S100B.SPP1.1 | CDH19 | 1.46 | CD.Prolif/Secretory.REG1B.MGST1 | UGT2A3 | 0.62 |
| CD.Endth/Art.SEMA3G.SSUH2 | ID1 | 2.58 | CD.Glial.S100B.SPP1.1 | XKR4 | 1.71 | CD.Prolif/Secretory.REG1B.MGST1 | MAL2 | 0.61 |
| CD.Endth/Cap.CD36.FLT1 | PLVAP | 3.58 | CD.Glial.S100B.SPP1.1 | SCN7A | 2.26 | CD.Prolif/Secretory.REG1B.MGST1 | DDAH1 | 0.56 |
| CD.Endth/Cap.CD36.FLT1 | FABP5 | 3.50 | CD.Glial.SPP1.NOV | CRYAB | 3.44 | CD.Prolif/Secretory.REG1B.MGST1 | CKMT1B | 0.52 |
| CD.Endth/Cap.CD36.FLT1 | SPARCL1 | 3.27 | CD.Glial.SPP1.NOV | SPP1 | 3.38 | CD.Prolif/Secretory.REG1B.MGST1 | FAM3D | 0.73 |
| CD.Endth/Cap.CD36.FLT1 | IGFBP7 | 2.97 | CD.Glial.SPP1.NOV | S100B | 2.97 | CD.Prolif/Secretory.REG1B.MGST1 | FABP1 | 1.54 |
| CD.Endth/Cap.CD36.FLT1 | CD320 | 2.82 | CD.Glial.SPP1.NOV | LGI4 | 2.79 | CD.Prolif/Secretory.REG1B.MGST1 | CKMT1A | 0.49 |
| CD.Endth/Cap.CD36.FLT1 | RAMP2 | 2.65 | CD.Glial.SPP1.NOV | PLP1 | 2.65 | CD.Prolif/Secretory.REG1B.MGST1 | C15orf48 | 2.23 |
| CD.Endth/Cap.CD36.FLT1 | A2M | 2.65 | CD.Glial.SPP1.NOV | MYOT | 2.29 | CD.Prolif/Secretory.REG1B.MGST1 | RAMP1 | 0.75 |
| CD.Endth/Cap.CD36.FLT1 | GNG11 | 2.64 | CD.Glial.SPP1.NOV | CDH19 | 2.18 | CD.Prolif/Secretory.REG1B.MGST1 | GSTA1 | 0.94 |
| CD.Endth/Cap.CD36.FLT1 | MGP | 2.48 | CD.Glial.SPP1.NOV | SCN7A | 2.18 | CD.Prolif/Secretory.REG1B.MGST1 | PRAP1 | 0.63 |
| CD.Endth/Cap.CD36.FLT1 | RAMP3 | 2.46 | CD.Glial.SPP1.NOV | SEMA3B | 2.15 | CD.Prolif/Secretory.REG1B.MGST1 | C2orf82 | 0.63 |
| CD.Endth/Cap.CD36.FLT1 | IGFBP4 | 2.41 | CD.Glial.SPP1.NOV | MAP1B | 1.88 | CD.Prolif/Secretory.REG1B.MGST1 | IFT172 | 0.63 |
| CD.Endth/Cap.CD36.FLT1 | GSN | 2.39 | CD.Glial.SPP1.NOV | NRXN1 | 1.76 | CD.Secretory.GSTA1.REG1B | DMBT1 | 2.48 |
| CD.Endth/Cap.CD36.FLT1 | CAV1 | 2.38 | CD.Glial.SPP1.NOV | DKK3 | 1.70 | CD.Secretory.GSTA1.REG1B | GSTA1 | 2.33 |
| CD.Endth/Cap.CD36.FLT1 | SLC9A3R2 | 2.37 | CD.Glial.SPP1.NOV | TSPAN11 | 1.70 | CD.Secretory.GSTA1.REG1B | ALDOB | 1.84 |
| CD.Endth/Cap.CD36.FLT1 | CD36 | 2.37 | CD.Glial.SPP1.NOV | NRN1 | 1.68 | CD.Secretory.GSTA1.REG1B | ADH1C | 1.71 |
| CD.Endth/Cap.CD36.FLT1 | PRSS23 | 2.35 | CD.Glial.SPP1.NOV | PAPPA | 1.65 | CD.Secretory.GSTA1.REG1B | ANPEP | 1.61 |
| CD.Endth/Cap.CD36.FLT1 | FLT1 | 2.25 | CD.Glial.SPP1.NOV | NTM | 1.65 | CD.Secretory.GSTA1.REG1B | AKR1C3 | 1.57 |
| CD.Endth/Cap.CD36.FLT1 | SPARC | 2.25 | CD.Glial.SPP1.NOV | NOV | 1.64 | CD.Secretory.GSTA1.REG1B | UGT2B17 | 1.56 |
| CD.Endth/Cap.CD36.FLT1 | VWF | 2.22 | CD.Glial.SPP1.NOV | PHLDA3 | 1.54 | CD.Secretory.GSTA1.REG1B | C19orf33 | 1.49 |
| CD.Endth/Cap.CD36.FLT1 | COL15A1 | 2.21 | CD.Glial.SPP1.NOV | MPZ | 1.53 | CD.Secretory.GSTA1.REG1B | CES2 | 1.49 |
| CD.Endth/Cap.CD36.FLT1 | HSPG2 | 2.20 | CD.Glial.SPP1.NOV | CLDN11 | 1.49 | CD.Secretory.GSTA1.REG1B | AGR3 | 1.37 |
| CD.Endth/Cap.CD36.FLT1 | CLDN5 | 2.19 | CD.Glial.SPP1.NOV | GFRA3 | 1.48 | CD.Secretory.GSTA1.REG1B | CHP2 | 1.21 |
| CD.Endth/Cap.CD36.FLT1 | TMEM88 | 2.15 | CD.Glial.SPP1.NOV | MIA | 1.44 | CD.Secretory.GSTA1.REG1B | MAOA | 1.20 |
| CD.Endth/Cap.CD36.FLT1 | IFI27 | 2.13 | CD.Glial.SPP1.NOV | SNCA | 1.31 | CD.Secretory.GSTA1.REG1B | LCN2 | 1.18 |
| CD.Endth/Cap.CD36.FLT1 | RNASE1 | 2.12 | CD.Glial.SPP1.NOV | NLGN4X | 1.30 | CD.Secretory.GSTA1.REG1B | PTGR1 | 1.06 |
| CD.Endth/Cap.CD36.FLT1 | TGFBR2 | 2.12 | CD.Glial.SPP1.NOV | TNC | 1.25 | CD.Secretory.GSTA1.REG1B | KCNJ13 | 1.02 |
| CD.Endth/Cap.CD36.FLT1 | ENG | 2.09 | CD.Glial.SPP1.NOV | FOXD3-AS1 | 1.23 | CD.Secretory.GSTA1.REG1B | FAM3B | 0.98 |
| CD.Endth/Cap.CD36.FLT1 | PECAM1 | 2.07 | CD.Glial.SPP1.NOV | CDH2 | 1.23 | CD.Secretory.GSTA1.REG1B | PRSS3 | 0.95 |
| CD.Endth/Cap.CD36.FLT1 | IFITM3 | 2.06 | CD.Glial.SPP1.NOV | XKR4 | 1.20 | CD.Secretory.GSTA1.REG1B | DHRS11 | 0.93 |
| CD.Endth/Cap.CD36.FLT1 | CRIP2 | 2.03 | CD.Glial.SPP1.NOV | SLC35F1 | 1.20 | CD.Secretory.GSTA1.REG1B | SI | 0.90 |
| CD.Endth/Cap.CD36.FLT1 | PLAT | 2.01 | CD.Glial.SPP1.NOV | KCNMB4 | 1.20 | CD.Secretory.GSTA1.REG1B | SLC39A5 | 0.87 |
| CD.Endth/Cap.CD36.FLT1 | ID1 | 2.00 | CD.Glial.SPP1.NOV | NGFR | 1.19 | CD.Secretory.GSTA1.REG1B | SLC44A4 | 0.78 |
| CD.Endth/Cap.CD36.FLT1 | VWA1 | 1.97 | CD.Glial.SPP1.NOV | HAND2 | 1.14 | CD.Secretory.GSTA1.REG1B | GSTA2 | 0.78 |
| CD.Endth/Cap.CD36.FLT1 | TM4SF1 | 1.96 | CD.Glial.SPP1.NOV | FST | 1.08 | CD.Secretory.GSTA1.REG1B | MTRNR2L1 | 0.76 |
| CD.Endth/Cap.CD36.FLT1 | NFIB | 1.94 | CD.Glial.SPP1.NOV | PRIMA1 | 1.03 | CD.Secretory.GSTA1.REG1B | TMEM45B | 0.76 |
| CD.Endth/Cap.CD36.FLT1 | EMCN | 1.94 | CD.Glial.SPP1.NOV | L1CAM | 0.99 | CD.Secretory.GSTA1.REG1B | C10orf99 | 0.73 |
| CD.Endth/Cap.CD36.FLT1 | ARHGAP29 | 1.94 | CD.Glial.SPP1.NOV | ZNF536 | 0.98 | CD.Secretory.GSTA1.REG1B | CCL15 | 0.70 |
| CD.Endth/Cap.CD36.FLT1 | C8orf4 | 1.92 | CD.Glial.SPP1.NOV | PMP2 | 0.96 | CD.Secretory.GSTA1.REG1B | MYO1A | 0.69 |
| CD.Endth/Cap.CD36.FLT1 | FAM167B | 1.89 | CD.Glial.SPP1.NOV | HAND2-AS1 | 0.91 | CD.Secretory.GSTA1.REG1B | URAD | 0.68 |
| CD.Endth/Cap.CD36.FLT1 | TM4SF18 | 1.88 | CD.Glial.SPP1.NOV | SBSPON | 0.91 | CD.Secretory.GSTA1.REG1B | UGT2A3 | 0.66 |
| CD.Endth/Cap.CD36.FLT1 | CALCRL | 1.85 | CD.Glial.SPP1.NOV | CADM3 | 0.90 | CD.Secretory.GSTA1.REG1B | NR1H4 | 0.66 |
| CD.Endth/Cap.CD36.FLT1 | EGFL7 | 1.85 | CD.Glial.SPP1.NOV | COL8A1 | 0.88 | CD.Secretory.GSTA1.REG1B | HSD11B2 | 0.63 |
| CD.Endth/Cap.CD36.FLT1 | PTRF | 1.84 | CD.Glial.SPP1.NOV | CADM4 | 0.87 | CD.Secretory.GSTA1.REG1B | REG3G | 0.49 |
| CD.Endth/Cap.CD36.FLT1 | ADGRL4 | 1.82 | CD.Glial.SPP1.NOV | PTPRZ1 | 0.82 | CD.Secretory.GSTA1.REG1B | PLLP | 0.45 |
| CD.Endth/Cap.CD36.FLT1 | RBP7 | 1.81 | CD.Glial.SPP1.NOV | SOX2 | 0.82 | CD.Secretory.GSTA1.REG1B | ARSE | 0.37 |
| CD.Endth/Cap.CD36.FLT1 | CD59 | 1.80 | CD.Glial.SPP1.NOV | SORCS1 | 0.81 | CD.Secretory.GSTA1.REG1B | DSG2 | 0.69 |
| CD.Endth/Cap.CD36.FLT1 | PLPP1 | 1.78 | CD.Glial.SPP1.NOV | NRXN3 | 0.78 | CD.Secretory.GSTA1.REG1B | CDX1 | 0.93 |
| CD.Endth/Cap.CD36.FLT1 | EPAS1 | 1.77 | CD.Glial.SPP1.NOV | LRRC4C | 0.76 | CD.Secretory.GSTA1.REG1B | REG1B | 3.68 |
| CD.Endth/Cap.CD36.FLT1 | LIFR | 1.76 | CD.Glial.SPP1.NOV | CADM2 | 0.71 | CD.Secretory.GSTA1.REG1B | ASS1 | 1.03 |
| CD.Endth/Cap.CD36.FLT1 | SDPR | 1.76 | CD.Glial.SPP1.NOV | LRRTM1 | 0.70 | CD.Secretory.GSTA1.REG1B | PPP1R1B | 1.00 |
| CD.Endth/Cap.CD36.FLT1 | PODXL | 1.74 | CD.Glial.SPP1.NOV | SHC4 | 0.67 | CD.Secretory.GSTA1.REG1B | DSP | 1.08 |
| CD.Endth/Cap.CD36.FLT1 | ID3 | 1.73 | CD.Glial.SPP1.NOV | CYTL1 | 0.65 | CD.Secretory.GSTA1.REG1B | TM4SF5 | 0.40 |
| CD.Endth/Cap.CD36.FLT1 | JAM2 | 1.72 | CD.Glial.SPP1.NOV | NCAM2 | 0.64 | CD.Secretory.GSTA1.REG1B | CMBL | 0.80 |
| CD.Endth/Cap.CD36.FLT1 | APP | 1.71 | CD.Glial.SPP1.NOV | CMTM5 | 0.63 | CD.Secretory.GSTA1.REG1B | FUT2 | 0.52 |
| CD.Endth/Cap.CD36.FLT1 | NPDC1 | 1.71 | CD.Glial.SPP1.NOV | B4GALNT1 | 0.60 | CD.Secretory.GSTA1.REG1B | CPS1 | 0.70 |
| CD.Endth/Cap.CD36.FLT1 | SPTBN1 | 1.70 | CD.Glial.SPP1.NOV | SHISA9 | 0.58 | CD.Secretory.GSTA1.REG1B | PLCB3 | 0.76 |
| CD.Endth/Cap.CD36.FLT1 | RBP5 | 1.70 | CD.Glial.SPP1.NOV | ART3 | 0.57 | CD.Secretory.GSTA1.REG1B | RAB25 | 0.74 |
| CD.Endth/Cap.CD36.FLT1 | COL4A1 | 1.70 | CD.Glial.SPP1.NOV | SYT10 | 0.55 | CD.Secretory.GSTA1.REG1B | CCL25 | 1.64 |
| CD.Endth/Cap.CD36.FLT1 | ESAM | 1.69 | CD.Glial.SPP1.NOV | LINC00461 | 0.51 | CD.Secretory.GSTA1.REG1B | TMEM54 | 1.11 |
| CD.Endth/Cap.CD36.FLT1 | CLEC14A | 1.68 | CD.Glial.SPP1.NOV | FOXD3 | 0.45 | CD.Secretory.GSTA1.REG1B | CDX2 | 0.79 |
| CD.Endth/Cap.CD36.FLT1 | CD34 | 1.64 | CD.Glial.SPP1.NOV | SLITRK2 | 0.38 | CD.Secretory.GSTA1.REG1B | DDC | 0.60 |
| CD.Endth/Cap.CD36.FLT1 | CA4 | 1.62 | CD.Glial.SPP1.NOV | FEZ1 | 1.17 | CD.Secretory.GSTA1.REG1B | SMIM24 | 0.84 |
| CD.Endth/Cap.CD36.FLT1 | PCAT19 | 1.61 | CD.Glial.SPP1.NOV | ST6GALNAC2 | 0.90 | CD.Secretory.GSTA1.REG1B | MT1H | 1.36 |
| CD.Endth/Cap.CD36.FLT1 | CYYR1 | 1.60 | CD.Glial.SPP1.NOV | SOX10 | 0.61 | CD.Secretory.GSTA1.REG1B | PKP2 | 0.70 |
| CD.Endth/Cap.CD36.FLT1 | ITGA6 | 1.58 | CD.Glial.SPP1.NOV | FSTL3 | 1.59 | CD.Secretory.GSTA1.REG1B | MGST1 | 1.14 |
| CD.Endth/Cap.CD36.FLT1 | EHD4 | 1.56 | CD.Glial.SPP1.NOV | FXYD1 | 2.56 | CD.Secretory.GSTA1.REG1B | ATP8B1 | 0.62 |
| CD.Endth/Cap.CD36.FLT1 | PLPP3 | 1.56 | CD.Glial.SPP1.NOV | ST6GALNAC5 | 0.50 | CD.Secretory.GSTA1.REG1B | PPP1R16A | 0.91 |
| CD.Endth/Cap.CD36.FLT1 | RDX | 1.54 | CD.Glial.SPP1.NOV | PDPN | 1.42 | CD.Secretory.GSTA1.REG1B | IGFBP2 | 0.77 |
| CD.Endth/Cap.CD36.FLT1 | CD93 | 1.52 | CD.Glial.SPP1.NRXN1 | CRYAB | 4.07 | CD.Secretory.GSTA1.REG1B | HSD17B2 | 0.39 |
| CD.Endth/Cap.CD36.FLT1 | INSR | 1.51 | CD.Glial.SPP1.NRXN1 | SPP1 | 3.23 | CD.Secretory.GSTA1.REG1B | FABP1 | 2.20 |
| CD.Endth/Cap.CD36.FLT1 | SLCO2A1 | 1.51 | CD.Glial.SPP1.NRXN1 | PLP1 | 2.93 | CD.Secretory.GSTA1.REG1B | PDE4C | 0.50 |
| CD.Endth/Cap.CD36.FLT1 | SORBS2 | 1.50 | CD.Glial.SPP1.NRXN1 | S100B | 2.85 | CD.Secretory.GSTA1.REG1B | C1orf106 | 0.46 |
| CD.Endth/Cap.CD36.FLT1 | TCF4 | 1.49 | CD.Glial.SPP1.NRXN1 | LG14 | 2.85 | CD.Secretory.GSTA1.REG1B | FAM3D | 0.91 |
| CD.Endth/Cap.CD36.FLT1 | STOM | 1.49 | CD.Glial.SPP1.NRXN1 | SCN7A | 2.61 | CD.Secretory.GSTA1.REG1B | CDH17 | 0.90 |
| CD.Endth/Cap.CD36.FLT1 | KDR | 1.47 | CD.Glial.SPP1.NRXN1 | NRXN1 | 2.50 | CD.Secretory.GSTA1.REG1B | VIL1 | 0.71 |
| CD.Endth/Cap.CD36.FLT1 | BCAM | 1.47 | CD.Glial.SPP1.NRXN1 | MPZ | 2.32 | CD.Secretory.REG1B.REG1A | REG1B | 3.51 |
| CD.Endth/Cap.CD36.FLT1 | NOSTRIN | 1.47 | CD.Glial.SPP1.NRXN1 | CDH19 | 2.15 | CD.Secretory.REG3G.IFI6 | LCN2 | 2.13 |
| CD.Endth/Cap.CD36.FLT1 | NOTCH4 | 1.47 | CD.Glial.SPP1.NRXN1 | GFRA3 | 2.01 | CD.Secretory.REG3G.IFI6 | REG3G | 1.50 |
| CD.Endth/Cap.CD36.FLT1 | CXCL12 | 1.47 | CD.Glial.SPP1.NRXN1 | MYOT | 1.84 | CD.Secretory.REG3G.IFI6 | PDZK1IP1 | 0.87 |
| CD.Endth/Cap.CD36.FLT1 | PTPRB | 1.46 | CD.Glial.SPP1.NRXN1 | NTM | 1.83 | CD.Secretory.REG3G.IFI6 | CEL | 0.46 |
| CD.Endth/Cap.CD36.FLT1 | ECSCR-1 | 1.45 | CD.Glial.SPP1.NRXN1 | TSPAN11 | 1.54 | CD.Secretory.REG3G.IFI6 | UGT2B17 | 1.69 |
| CD.Endth/Cap.CD36.FLT1 | NRP1 | 1.44 | CD.Glial.SPP1.NRXN1 | NGFR | 1.44 | CD.T.ANXA1.IL7R | IL7R | 1.86 |
| CD.Endth/Cap.CD36.FLT1 | FCN3 | 1.43 | CD.Glial.SPP1.NRXN1 | XKR4 | 1.43 | CD.T.ANXA1.IL7R | ANXA1 | 1.61 |
| CD.Endth/Cap.CD36.FLT1 | THBD | 1.43 | CD.Glial.SPP1.NRXN1 | CADM2 | 1.43 | CD.T.ANXA1.IL7R | CREM | 1.48 |
| CD.Endth/Cap.CD36.FLT1 | LDB2 | 1.42 | CD.Glial.SPP1.NRXN1 | PRIMA1 | 1.38 | CD.T.ANXA1.IL7R | TPT1 | 1.20 |
| CD.Endth/Cap.CD36.FLT1 | CAV2 | 1.41 | CD.Glial.SPP1.NRXN1 | SOX2 | 1.28 | CD.T.ANXA1.IL7R | KLRB1 | 1.20 |
| CD.Endth/Cap.CD36.FLT1 | HYAL2 | 1.40 | CD.Glial.SPP1.NRXN1 | COL28A1 | 1.26 | CD.T.ANXA1.IL7R | RPL9 | 0.68 |
| CD.Endth/Cap.CD36.FLT1 | COL4A2 | 1.40 | CD.Glial.SPP1.NRXN1 | FOXD3-AS1 | 1.26 | CD.T.ANXA1.IL7R | EEF1A1 | 0.67 |
| CD.Endth/Cap.CD36.FLT1 | MMRN2 | 1.39 | CD.Glial.SPP1.NRXN1 | SOX10 | 1.24 | CD.T.ANXA1.IL7R | RPLP1 | 0.61 |
| CD.Endth/Cap.CD36.FLT1 | TIMP3 | 1.38 | CD.Glial.SPP1.NRXN1 | L1CAM | 1.24 | CD.T.ANXA1.IL7R | SARAF | 1.02 |
| CD.Endth/Cap.CD36.FLT1 | MGLL | 1.38 | CD.Glial.SPP1.NRXN1 | ART3 | 1.24 | CD.T.ANXA1.IL7R | GIMAP7 | 1.12 |
| CD.Endth/Cap.CD36.FLT1 | PCDH17 | 1.37 | CD.Glial.SPP1.NRXN1 | SYT10 | 1.00 | CD.T.ANXA1.IL7R | RPL30 | 0.68 |
| CD.Endth/Cap.CD36.FLT1 | S100A13 | 1.36 | CD.Glial.SPP1.NRXN1 | PCSK2 | 0.96 | CD.T.ANXA1.IL7R | RPS12 | 0.61 |
| CD.Endth/Cap.CD36.FLT1 | ETS2 | 1.34 | CD.Glial.SPP1.NRXN1 | LRRTM1 | 0.95 | CD.T.CARD16.GB2 | IL32 | 1.84 |
| CD.Endth/Cap.CD36.FLT1 | NQO1 | 1.33 | CD.Glial.SPP1.NRXN1 | NKAIN3 | 0.94 | CD.T.CARD16.GB2 | RGS1 | 1.37 |
| CD.Endth/Cap.CD36.FLT1 | CNN3 | 1.32 | CD.Glial.SPP1.NRXN1 | MGAT4C | 0.92 | CD.T.CARD16.GB2 | CD3D | 1.22 |
| CD.Endth/Cap.CD36.FLT1 | CDH5 | 1.32 | CD.Glial.SPP1.NRXN1 | HAND2 | 1.09 | CD.T.CARD16.GB2 | B2M | 0.56 |
| CD.Endth/Cap.CD36.FLT1 | CFH | 1.32 | CD.Glial.SPP1.NRXN1 | CYR61 | 3.06 | CD.T.CARD16.GB2 | TRAC | 1.14 |
| CD.Endth/Cap.CD36.FLT1 | HEG1 | 1.30 | CD.Glial.SPP1.NRXN1 | SEMA3B | 2.30 | CD.T.CCL20.IFNG | CCL20 | 3.79 |
| CD.Endth/Cap.CD36.FLT1 | SYNPO | 1.29 | CD.Glial.XKR4.S100B | S100B | 3.52 | CD.T.CCL20.IL22 | IL22 | 2.33 |
| CD.Endth/Cap.CD36.FLT1 | ITGA1 | 1.28 | CD.Glial.XKR4.S100B | CRYAB | 3.42 | CD.T.CCL20.RORA | KLRB1 | 1.66 |
| CD.Endth/Cap.CD36.FLT1 | SOX18 | 1.27 | CD.Glial.XKR4.S100B | SPP1 | 3.40 | CD.T.CCL20.RORA | S100A4 | 1.65 |
| CD.Endth/Cap.CD36.FLT1 | IGFBP3 | 1.27 | CD.Glial.XKR4.S100B | SCN7A | 3.05 | CD.T.CCL20.RORA | CD3D | 1.42 |
| CD.Endth/Cap.CD36.FLT1 | IGFBP6 | 1.25 | CD.Glial.XKR4.S100B | PLP1 | 3.04 | CD.T.CCL20.RORA | CD2 | 1.41 |
| CD.Endth/Cap.CD36.FLT1 | APLNR | 1.25 | CD.Glial.XKR4.S100B | NRXN1 | 2.68 | CD.T.CCL20.RORA | AC092580.4 | 1.28 |
| CD.Endth/Cap.CD36.FLT1 | S100A16 | 1.25 | CD.Glial.XKR4.S100B | LGI4 | 2.53 | CD.T.CCL20.RORA | RORA | 1.28 |
| CD.Endth/Cap.CD36.FLT1 | PRKCDBP | 1.24 | CD.Glial.XKR4.S100B | XKR4 | 2.35 | CD.T.CCL20.RORA | IL7R | 1.24 |
| CD.Endth/Cap.CD36.FLT1 | LPAR6 | 1.22 | CD.Glial.XKR4.S100B | MPZ | 2.18 | CD.T.CCL20.RORA | IL32 | 1.16 |
| CD.Endth/Cap.CD36.FLT1 | WWTR1 | 1.21 | CD.Glial.XKR4.S100B | CDH19 | 2.17 | CD.T.CCL20.RORA | CD52 | 1.14 |
| CD.Endth/Cap.CD36.FLT1 | ADGRF5 | 1.20 | CD.Glial.XKR4.S100B | MYOT | 2.06 | CD.T.CCL20.RORA | PTPRC | 1.08 |
| CD.Endth/Cap.CD36.FLT1 | EFNA1 | 1.17 | CD.Glial.XKR4.S100B | GFRA3 | 2.01 | CD.T.CCL20.RORA | TRAC | 1.06 |
| CD.Endth/Cap.CD36.FLT1 | EMP2 | 1.16 | CD.Glial.XKR4.S100B | NLGN4X | 1.82 | CD.T.CCL20.RORA | B2M | 0.76 |
| CD.Endth/Cap.CD36.FLT1 | IL3RA | 1.15 | CD.Glial.XKR4.S100B | PTPRZ1 | 1.59 | CD.T.CCL20.RORA | TMSB4X | 0.70 |
| CD.Endth/Cap.CD36.FLT1 | SERPINH1 | 1.15 | CD.Glial.XKR4.S100B | COL28A1 | 1.50 | CD.T.CCL20.RORA | SH3BGRL3 | 1.14 |
| CD.Endth/Cap.CD36.FLT1 | C1orf54 | 1.14 | CD.Glial.XKR4.S100B | NOV | 1.49 | CD.T.CCL20.RORA | CCL5 | 1.16 |
| CD.Endth/Cap.CD36.FLT1 | LIMCH1 | 1.13 | CD.Glial.XKR4.S100B | COL21A1 | 1.47 | CD.T.CCL20.RORA | GZMA | 1.24 |
| CD.Endth/Cap.CD36.FLT1 | IL33 | 1.13 | CD.Glial.XKR4.S100B | CYTL1 | 1.46 | CD.T.CCL4.GMZK | GZMK | 2.60 |
| CD.Endth/Cap.CD36.FLT1 | MKL2 | 1.12 | CD.Glial.XKR4.S100B | CADM2 | 1.43 | CD.T.CCL4.GMZK | CCL5 | 1.99 |
| CD.Endth/Cap.CD36.FLT1 | TSPAN7 | 1.12 | CD.Glial.XKR4.S100B | ST6GALNAC2 | 1.41 | CD.T.CCL4.GMZK | CCL4 | 1.95 |
| CD.Endth/Cap.CD36.FLT1 | ADAM15 | 1.12 | CD.Glial.XKR4.S100B | PMP2 | 1.29 | CD.T.CCL4.GMZK | CST7 | 1.86 |
| CD.Endth/Cap.CD36.FLT1 | FAM198B | 1.11 | CD.Glial.XKR4.S100B | FOXD3-AS1 | 1.25 | CD.T.CCL4.GMZK | DUSP2 | 1.65 |
| CD.Endth/Cap.CD36.FLT1 | ACVRL1 | 1.09 | CD.Glial.XKR4.S100B | PRIMA1 | 1.23 | CD.T.CCL4.GMZK | GZMA | 1.59 |
| CD.Endth/Cap.CD36.FLT1 | TINAGL1 | 1.09 | CD.Glial.XKR4.S100B | MGAT4C | 1.23 | CD.T.CCL4.GMZK | NKG7 | 1.53 |
| CD.Endth/Cap.CD36.FLT1 | COX7A1 | 1.09 | CD.Glial.XKR4.S100B | UG0898H09 | 1.02 | CD.T.CCL4.GMZK | IFNG | 1.46 |
| CD.Endth/Cap.CD36.FLT1 | PLCB1 | 1.08 | CD.Glial.XKR4.S100B | SOX10 | 0.74 | CD.T.CCL4.GMZK | DUSP4 | 1.34 |
| CD.Endth/Cap.CD36.FLT1 | NUAK1 | 1.08 | CD.Glial.XKR4.S100B | LGI1 | 0.82 | CD.T.CCL4.GMZK | RGS1 | 1.20 |
| CD.Endth/Cap.CD36.FLT1 | ACE | 1.08 | CD.Glial.XKR4.S100B | PCDH9 | 1.85 | CD.T.CCL4.GMZK | CD8A | 1.19 |
| CD.Endth/Cap.CD36.FLT1 | CTNNAL1 | 1.08 | CD.Glial.XKR4.S100B | FXYD1 | 3.01 | CD.T.CCL4.GMZK | CD3D | 1.15 |
| CD.Endth/Cap.CD36.FLT1 | NES | 1.07 | CD.Glial.XKR4.S100B | L1CAM | 1.21 | CD.T.CCL4.GMZK | TRGC2 | 1.14 |
| CD.Endth/Cap.CD36.FLT1 | TMEM204 | 1.05 | CD.Goblet.BPIFB1.AQP5 | PGC | 5.92 | CD.T.CCL4.GMZK | TRAC | 1.08 |
| CD.Endth/Cap.CD36.FLT1 | NKX2-3 | 1.05 | CD.Goblet.BPIFB1.AQP5 | LCN2 | 5.40 | CD.T.CCL4.GMZK | LAG3 | 1.06 |
| CD.Endth/Cap.CD36.FLT1 | PKP4 | 1.05 | CD.Goblet.BPIFB1.AQP5 | ZG16B | 4.57 | CD.T.CCL4.GMZK | IL32 | 1.04 |
| CD.Endth/Cap.CD36.FLT1 | ROBO4 | 1.04 | CD.Goblet.BPIFB1.AQP5 | MUC6 | 4.39 | CD.T.CCL4.GMZK | HCST | 1.03 |
| CD.Endth/Cap.CD36.FLT1 | MALL | 1.03 | CD.Goblet.BPIFB1.AQP5 | BPIFB1 | 3.64 | CD.T.CCL4.GMZK | SH2D1A | 1.01 |
| CD.Endth/Cap.CD36.FLT1 | CRIM1 | 1.03 | CD.Goblet.BPIFB1.AQP5 | MSMB | 2.45 | CD.T.CCL4.GMZK | TRBC2 | 0.99 |
| CD.Endth/Cap.CD36.FLT1 | EPHX1 | 1.03 | CD.Goblet.BPIFB1.AQP5 | MUC5B | 2.36 | CD.T.CCL4.GMZK | CXCR4 | 0.97 |
| CD.Endth/Cap.CD36.FLT1 | SLC14A1 | 1.03 | CD.Goblet.BPIFB1.AQP5 | RP13-870H17.3 | 2.04 | CD.T.CCL4.GMZK | CD3E | 0.97 |
| CD.Endth/Cap.CD36.FLT1 | CALD1 | 1.03 | CD.Goblet.BPIFB1.AQP5 | S100P | 1.98 | CD.T.CCL4.GMZK | CD2 | 0.95 |
| CD.Endth/Cap.CD36.FLT1 | KANK3 | 1.03 | CD.Goblet.BPIFB1.AQP5 | SAA1 | 1.92 | CD.T.CCL4.GMZK | CD69 | 0.87 |
| CD.Endth/Cap.CD36.FLT1 | BMPR2 | 1.01 | CD.Goblet.BPIFB1.AQP5 | LIPF | 1.77 | CD.T.CCL4.GMZK | BTG1 | 0.80 |
| CD.Endth/Cap.CD36.FLT1 | UACA | 1.00 | CD.Goblet.BPIFB1.AQP5 | TCN1 | 1.67 | CD.T.CCL4.GMZK | HLA-A | 0.75 |
| CD.Endth/Cap.CD36.FLT1 | MYCT1 | 1.00 | CD.Goblet.BPIFB1.AQP5 | CA2 | 1.65 | CD.T.CCL4.GMZK | HLA-B | 0.73 |
| CD.Endth/Cap.CD36.FLT1 | TMTC1 | 0.99 | CD.Goblet.BPIFB1.AQP5 | AQP5 | 1.63 | CD.T.CCL4.GMZK | H3F3B | 0.62 |
| CD.Endth/Cap.CD36.FLT1 | EMP1 | 0.98 | CD.Goblet.BPIFB1.AQP5 | CLDN2 | 1.59 | CD.T.CCL4.GMZK | HLA-C | 0.59 |
| CD.Endth/Cap.CD36.FLT1 | FAM110D | 0.97 | CD.Goblet.BPIFB1.AQP5 | C6orf58 | 1.42 | CD.T.CCL4.GMZK | B2M | 0.57 |
| CD.Endth/Cap.CD36.FLT1 | SNCG | 0.97 | CD.Goblet.BPIFB1.AQP5 | MIA | 1.40 | CD.T.CCL4.GMZK | TMSB4X | 0.48 |
| CD.Endth/Cap.CD36.FLT1 | PLS3 | 0.92 | CD.Goblet.BPIFB1.AQP5 | SLPI | 1.37 | CD.T.CCL4.GMZK | MALAT1 | 0.36 |
| CD.Endth/Cap.CD36.FLT1 | PTPRG | 0.92 | CD.Goblet.BPIFB1.AQP5 | COL11A2 | 1.25 | CD.T.CCL4.GMZK | SRGN | 0.56 |
| CD.Endth/Cap.CD36.FLT1 | GPRC5B | 0.92 | CD.Goblet.BPIFB1.AQP5 | RP11- | 1.22 | CD.T.CCL4.GMZK | JUNB | 0.84 |
| | | | | 469H8.6 | | | | |
| CD.Endth/Cap.CD36.FLT1 | LMCD1 | 0.91 | CD.Goblet.BPIFB1.AQP5 | MMP7 | 1.05 | CD.T.CCL4.GMZK | CD8B | 0.99 |
| CD.Endth/Cap.CD36.FLT1 | TIE1 | 0.90 | CD.Goblet.BPIFB1.AQP5 | CXCL17 | 1.01 | CD.T.CCL4.GMZK | PTPRC | 0.72 |
| CD.Endth/Cap.CD36.FLT1 | RAI14 | 0.90 | CD.Goblet.BPIFB1.AQP5 | SAA2 | 0.99 | CD.T.CCL4.GMZK | CRTAM | 1.05 |
| CD.Endth/Cap.CD36.FLT1 | ERG | 0.90 | CD.Goblet.BPIFB1.AQP5 | VSIG2 | 0.96 | CD.T.CCR7.GPR183 | TRBC1 | 1.14 |
| CD.Endth/Cap.CD36.FLT1 | CXorf36 | 0.90 | CD.Goblet.BPIFB1.AQP5 | ODAM | 0.85 | CD.T.CCR7.GPR183 | TRAC | 1.06 |
| CD.Endth/Cap.CD36.FLT1 | LINC01235 | 0.89 | CD.Goblet.BPIFB1.AQP5 | NOTUM | 0.69 | CD.T.CCR7.GPR183 | FYB | 0.99 |
| CD.Endth/Cap.CD36.FLT1 | FAM107A | 0.87 | CD.Goblet.BPIFB1.AQP5 | RNASE1 | 2.60 | CD.T.CCR7.GPR183 | TRBC2 | 0.97 |
| CD.Endth/Cap.CD36.FLT1 | NOV | 0.87 | CD.Goblet.BPIFB1.AQP5 | CRISP3 | 1.13 | CD.T.CCR7.GPR183 | CD3D | 0.91 |
| CD.Endth/Cap.CD36.FLT1 | TMEM255B | 0.85 | CD.Goblet.BPIFB1.AQP5 | FAM3D | 2.33 | CD.T.CCR7.GPR183 | JUNB | 0.87 |
| CD.Endth/Cap.CD36.FLT1 | TSPAN12 | 0.85 | CD.Goblet.CENPA.UBE2C | CLCA1 | 3.57 | CD.T.CCR7.GPR183 | CD3E | 0.86 |
| CD.Endth/Cap.CD36.FLT1 | DLC1 | 0.85 | CD.Goblet.CENPA.UBE2C | ITLN1 | 2.96 | CD.T.CCR7.GPR183 | CD7 | 0.77 |
| CD.Endth/Cap.CD36.FLT1 | PALMD | 0.83 | CD.Goblet.CENPA.UBE2C | REG4 | 2.67 | CD.T.CCR7.GPR183 | CD2 | 0.74 |
| CD.Endth/Cap.CD36.FLT1 | SOX17 | 0.83 | CD.Goblet.CENPA.UBE2C | FCGBP | 2.31 | CD.T.CCR7.GPR183 | CXCR4 | 0.69 |
| CD.Endth/Cap.CD36.FLT1 | TEK | 0.80 | CD.Goblet.CENPA.UBE2C | RETNLB | 2.05 | CD.T.CCR7.GPR183 | BTG1 | 0.62 |
| CD.Endth/Cap.CD36.FLT1 | BTNL9 | 0.80 | CD.Goblet.CENPA.UBE2C | UBE2C | 1.89 | CD.T.CCR7.GPR183 | PTPRC | 0.60 |
| CD.Endth/Cap.CD36.FLT1 | TNS2 | 0.80 | CD.Goblet.CENPA.UBE2C | ST6GALNAC1 | 1.65 | CD.T.CCR7.GPR183 | MALAT1 | 0.55 |
| CD.Endth/Cap.CD36.FLT1 | LIMS2 | 0.79 | CD.Goblet.CENPA.UBE2C | TOP2A | 1.50 | CD.T.CCR7.GPR183 | IL32 | 0.53 |
| CD.Endth/Cap.CD36.FLT1 | RASIP1 | 0.79 | CD.Goblet.CENPA.UBE2C | HEPACAM2 | 1.17 | CD.T.CCR7.GPR183 | RPS29 | 0.48 |
| CD.Endth/Cap.CD36.FLT1 | NRN1 | 0.77 | CD.Goblet.CENPA.UBE2C | REP15 | 1.12 | CD.T.CCR7.GPR183 | RPS27 | 0.47 |
| CD.Endth/Cap.CD36.FLT1 | GJA1 | 0.77 | CD.Goblet.CENPA.UBE2C | LRRC26 | 1.06 | CD.T.CCR7.GPR183 | RPS15A | 0.44 |
| CD.Endth/Cap.CD36.FLT1 | FAM13C | 0.76 | CD.Goblet.CENPA.UBE2C | FOXA3 | 1.04 | CD.T.CCR7.GPR183 | RPS25 | 0.43 |
| CD.Endth/Cap.CD36.FLT1 | LRRC32 | 0.76 | CD.Goblet.CENPA.UBE2C | ATOH1 | 1.04 | CD.T.CCR7.GPR183 | RPL30 | 0.43 |
| CD.Endth/Cap.CD36.FLT1 | FXYD6 | 0.76 | CD.Goblet.CENPA.UBE2C | CREB3L1 | 1.01 | CD.T.CCR7.GPR183 | RPLP2 | 0.43 |
| CD.Endth/Cap.CD36.FLT1 | HECW2 | 0.75 | CD.Goblet.CENPA.UBE2C | CCNB2 | 0.97 | CD.T.CCR7.GPR183 | RPL32 | 0.43 |
| CD.Endth/Cap.CD36.FLT1 | SHE | 0.74 | CD.Goblet.CENPA.UBE2C | SPDEF | 0.96 | CD.T.CCR7.GPR183 | RPL31 | 0.42 |
| CD.Endth/Cap.CD36.FLT1 | FZD4 | 0.72 | CD.Goblet.CENPA.UBE2C | CENPA | 0.94 | CD.T.CCR7.GPR183 | RPL34 | 0.41 |
| CD.Endth/Cap.CD36.FLT1 | HSPA12B | 0.72 | CD.Goblet.CENPA.UBE2C | CCNA2 | 0.94 | CD.T.CCR7.GPR183 | RPL21 | 0.40 |
| CD.Endth/Cap.CD36.FLT1 | MCF2L | 0.71 | CD.Goblet.CENPA.UBE2C | ANXA13 | 0.79 | CD.T.CCR7.GPR183 | RPS3 | 0.37 |
| CD.Endth/Cap.CD36.FLT1 | IQCK | 0.70 | CD.Goblet.CENPA.UBE2C | PBK | 0.78 | CD.T.CCR7.GPR183 | RPL35A | 0.36 |
| CD.Endth/Cap.CD36.FLT1 | NDST1 | 0.69 | CD.Goblet.CENPA.UBE2C | RP11- | 0.65 | CD.T.CCR7.GPR183 | RPS12 | 0.35 |
| | | | | 234B24.2 | | | | |
| CD.Endth/Cap.CD36.FLT1 | ARHGEF15 | 0.69 | CD.Goblet.CENPA.UBE2C | TPSG1 | 0.60 | CD.T.CCR7.GPR183 | RPS6 | 0.34 |
| CD.Endth/Cap.CD36.FLT1 | TMEM47 | 0.69 | CD.Goblet.CENPA.UBE2C | LINC00261 | 0.56 | CD.T.CCR7.GPR183 | RPL11 | 0.34 |
| CD.Endth/Cap.CD36.FLT1 | ADCY4 | 0.68 | CD.Goblet.CENPA.UBE2C | RAP1GAP | 0.51 | CD.T.CCR7.GPR183 | RPS14 | 0.34 |
| CD.Endth/Cap.CD36.FLT1 | PDE1C | 0.67 | CD.Goblet.CENPA.UBE2C | ABCA4 | 0.51 | CD.T.CCR7.GPR183 | RPL13A | 0.31 |
| CD.Endth/Cap.CD36.FLT1 | GPR146 | 0.65 | CD.Goblet.CENPA.UBE2C | MB | 0.49 | CD.T.CCR7.GPR183 | RPL13 | 0.29 |
| CD.Endth/Cap.CD36.FLT1 | RAPGEF4 | 0.64 | CD.Goblet.CENPA.UBE2C | BIRC5 | 1.04 | CD.T.CCR7.GPR183 | RPS28 | 0.33 |
| CD.Endth/Cap.CD36.FLT1 | ATOH8 | 0.64 | CD.Goblet.CENPA.UBE2C | RNASE1 | 1.45 | CD.T.CCR7.GPR183 | RPL41 | 0.30 |
| CD.Endth/Cap.CD36.FLT1 | KIAA1462 | 0.61 | CD.Goblet.CENPA.UBE2C | DEPDC1 | 0.68 | CD.T.CCR7.GPR183 | ICOS | 0.84 |
| CD.Endth/Cap.CD36.FLT1 | GALNT18 | 0.57 | CD.Goblet.CENPA.UBE2C | MKI67 | 1.29 | CD.T.CCR7.GPR183 | RPL19 | 0.32 |
| CD.Endth/Cap.CD36.FLT1 | EXOC3L2 | 0.55 | CD.Goblet.CENPA.UBE2C | HMMR | 1.02 | CD.T.CCR7.GPR183 | ITM2A | 0.87 |
| CD.Endth/Cap.CD36.FLT1 | RASSF9 | 0.47 | CD.Goblet.CENPA.UBE2C | HES6 | 1.47 | CD.T.CCR7.SELL | CCR7 | 1.18 |
| CD.Endth/Cap.CD36.FLT1 | HTRA1 | 0.84 | CD.Goblet.CENPA.UBE2C | ECT2 | 0.80 | CD.T.CCR7.SELL | KLF2 | 1.11 |
| CD.Endth/Cap.CD36.FLT1 | CFI | 0.99 | CD.Goblet.CENPA.UBE2C | CEP55 | 0.76 | CD.T.CCR7.SELL | RPS4Y1 | 1.01 |
| CD.Endth/Cap.CD36.FLT1 | TXNIP | 1.95 | CD.Goblet.CENPA.UBE2C | ANLN | 0.55 | CD.T.CCR7.SELL | GIMAP7 | 0.95 |
| CD.Endth/Cap.CD36.FLT1 | PREX2 | 0.57 | CD.Goblet.CENPA.UBE2C | FXYD3 | 1.61 | CD.T.CCR7.SELL | LDHB | 0.94 |
| CD.Endth/Cap.CD36.FLT1 | RAB13 | 1.31 | CD.Goblet.CENPA.UBE2C | GALNT5 | 0.57 | CD.T.CCR7.SELL | LEF1 | 0.89 |
| CD.Endth/Cap.CD36.FLT1 | FKBP9 | 0.82 | CD.Goblet.FCGBP.CLCA1 | SPINK4 | 4.18 | CD.T.CCR7.SELL | RPS12 | 0.87 |
| CD.Endth/Cap.CD36.FLT1 | NOVA2 | 0.49 | CD.Goblet.FCGBP.CLCA1 | CLCA1 | 3.76 | CD.T.CCR7.SELL | RPS10 | 0.85 |
| CD.Endth/Cap.CD36.FLT1 | FGD5 | 0.60 | CD.Goblet.FCGBP.CLCA1 | FCGBP | 3.72 | CD.T.CCR7.SELL | SELL | 0.85 |
| CD.Endth/Cap.CD36.FLT1 | TNFSF10 | 1.52 | CD.Goblet.FCGBP.CLCA1 | ITLN1 | 2.27 | CD.T.CCR7.SELL | RPL31 | 0.81 |
| CD.Endth/Cap.CD36.FLT1 | SOX7 | 0.55 | CD.Goblet.FCGBP.CLCA1 | REG4 | 2.02 | CD.T.CCR7.SELL | RPL34 | 0.81 |
| CD.Endth/Cap.CD36.FLT1 | RAPGEF5 | 0.76 | CD.Goblet.FCGBP.CLCA1 | ST6GALNAC1 | 1.61 | CD.T.CCR7.SELL | RPS15A | 0.80 |
| CD.Endth/Cap.CD36.FLT1 | DPYSL3 | 0.70 | CD.Goblet.FCGBP.CLCA1 | REP15 | 1.31 | CD.T.CCR7.SELL | RPL30 | 0.79 |
| CD.Endth/Cap.CD36.FLT1 | TSC22D1 | 1.40 | CD.Goblet.FCGBP.CLCA1 | APOA1 | 0.64 | CD.T.CCR7.SELL | RPLP2 | 0.79 |
| CD.Endth/Cap.CD36.FLT1 | RAPGEF3 | 0.66 | CD.Goblet.FCGBP.CLCA1 | ATOH1 | 0.73 | CD.T.CCR7.SELL | RPL21 | 0.79 |
| CD.Endth/Cap.CD36.FLT1 | RHOJ | 0.61 | CD.Goblet.FCGBP.CLCA1 | RETNLB | 1.45 | CD.T.CCR7.SELL | RPS13 | 0.79 |
| CD.Endth/Cap.CD36.FLT1 | THSD7A | 0.71 | CD.Goblet.FCGBP.CLCA1 | BCAS1 | 1.14 | CD.T.CCR7.SELL | RPL32 | 0.78 |
| CD.Endth/Cap.CD36.FLT1 | RASAL2 | 0.64 | CD.Goblet.FCGBP.ITLN1 | FCGBP | 3.24 | CD.T.CCR7.SELL | RPS6 | 0.77 |
| CD.Endth/Cap.CD36.FLT1 | VAMP5 | 1.64 | CD.Goblet.FCGBP.ITLN1 | CLCA1 | 3.18 | CD.T.CCR7.SELL | RPS25 | 0.77 |
| CD.Endth/Cap.CD36.FLT1 | PTPRM | 0.66 | CD.Goblet.FCGBP.ITLN1 | ITLN1 | 2.79 | CD.T.CCR7.SELL | RPS27 | 0.76 |
| CD.Endth/Cap.CD36.FLT1 | NPR1 | 0.45 | CD.Goblet.FCGBP.ITLN1 | REG4 | 2.42 | CD.T.CCR7.SELL | CD3G | 0.75 |
| CD.Endth/Cap.CD36.FLT1 | HSPB1 | 1.54 | CD.Goblet.FCGBP.ITLN1 | REP15 | 1.60 | CD.T.CCR7.SELL | RPL38 | 0.74 |
| CD.Endth/Cap.CD36.FLT1 | CLIC2 | 0.87 | CD.Goblet.FCGBP.ITLN1 | RP11- | 0.90 | CD.T.CCR7.SELL | RPL9 | 0.73 |
| | | | | 234B24.2 | | | | |
| CD.Endth/Cap.CD36.FLT1 | S1PR1 | 0.80 | CD.Goblet.FCGBP.ITLN1 | RETNLB | 1.86 | CD.T.CCR7.SELL | RPS29 | 0.73 |
| CD.Endth/Cap.CD36.FLT1 | CYP26B1 | 0.65 | CD.Goblet.FCGBP.ITLN1 | SPINK4 | 3.40 | CD.T.CCR7.SELL | RPS27A | 0.72 |
| CD.Endth/Cap.CD36.FLT1 | PPFIBP1 | 0.83 | CD.Goblet.FCGBP.SPINK4 | SPINK4 | 3.57 | CD.T.CCR7.SELL | RPS8 | 0.72 |
| CD.Endth/Lymph.CCL21.DKK3 | CCL21 | 4.74 | CD.Goblet.FCGBP.SPINK4 | FCGBP | 3.51 | CD.T.CCR7.SELL | RPL35A | 0.72 |
| CD.Endth/Lymph.CCL21.DKK3 | LYVE1 | 2.86 | CD.Goblet.FCGBP.SPINK4 | TFF3 | 3.29 | CD.T.CCR7.SELL | RPS14 | 0.72 |
| CD.Endth/Lymph.CCL21.DKK3 | MEDAG | 1.11 | CD.Goblet.FCGBP.SPINK4 | CLCA1 | 2.61 | CD.T.CCR7.SELL | RPL22 | 0.71 |
| CD.Endth/Lymph.CCL21.DKK3 | MMRN1 | 2.46 | CD.Goblet.FCGBP.SPINK4 | REG4 | 2.47 | CD.T.CCR7.SELL | CD7 | 0.71 |
| CD.Endth/Lymph.CCL21.DKK3 | CCL14 | 1.90 | CD.Goblet.FCGBP.SPINK4 | ITLN1 | 2.21 | CD.T.CCR7.SELL | RPS21 | 0.71 |
| CD.Endth/Lymph.CCL21.LYVE1 | CCL21 | 5.18 | CD.Goblet.FCGBP.SPINK4 | REP15 | 1.07 | CD.T.CCR7.SELL | RPL36A | 0.71 |
| CD.Endth/Lymph.CCL21.LYVE1 | TFPI | 3.85 | CD.Goblet.HES6.COLCA2 | HEPACAM2 | 1.10 | CD.T.CCR7.SELL | RPL39 | 0.70 |
| CD.Endth/Lymph.CCL21.LYVE1 | MMRN1 | 3.39 | CD.Goblet.ITLN1.CLCA1 | TFF3 | 4.35 | CD.T.CCR7.SELL | EEF1A1 | 0.70 |
| CD.Endth/Lymph.CCL21.LYVE1 | LYVE1 | 2.99 | CD.Goblet.ITLN1.CLCA1 | CLCA1 | 4.21 | CD.T.CCR7.SELL | RPL4 | 0.69 |
| CD.Endth/Lymph.CCL21.LYVE1 | EFEMP1 | 2.89 | CD.Goblet.ITLN1.CLCA1 | SPINK4 | 3.87 | CD.T.CCR7.SELL | RPS3A | 0.69 |
| CD.Endth/Lymph.CCL21.LYVE1 | GNG11 | 2.88 | CD.Goblet.ITLN1.CLCA1 | REG4 | 3.73 | CD.T.CCR7.SELL | RPL7 | 0.68 |
| CD.Endth/Lymph.CCL21.LYVE1 | RBP1 | 2.62 | CD.Goblet.ITLN1.CLCA1 | ITLN1 | 3.69 | CD.T.CCR7.SELL | CD3E | 0.68 |
| CD.Endth/Lymph.CCL21.LYVE1 | SDPR | 2.47 | CD.Goblet.ITLN1.CLCA1 | AGR2 | 3.33 | CD.T.CCR7.SELL | RPL13 | 0.68 |
| CD.Endth/Lymph.CCL21.LYVE1 | PROX1 | 2.36 | CD.Goblet.ITLN1.CLCA1 | FCGBP | 3.17 | CD.T.CCR7.SELL | RPL37 | 0.68 |
| CD.Endth/Lymph.CCL21.LYVE1 | PPFIBP1 | 2.20 | CD.Goblet.ITLN1.CLCA1 | ZG16 | 2.80 | CD.T.CCR7.SELL | RPS26 | 0.67 |
| CD.Endth/Lymph.CCL21.LYVE1 | ECSCR-1 | 2.15 | CD.Goblet.ITLN1.CLCA1 | LGALS4 | 2.70 | CD.T.CCR7.SELL | RPS3 | 0.67 |
| CD.Endth/Lymph.CCL21.LYVE1 | ARHGAP29 | 1.86 | CD.Goblet.ITLN1.CLCA1 | RETNLB | 2.37 | CD.T.CCR7.SELL | RPL27 | 0.67 |
| CD.Endth/Lymph.CCL21.LYVE1 | NRP2 | 1.82 | CD.Goblet.ITLN1.CLCA1 | FXYD3 | 2.11 | CD.T.CCR7.SELL | TRAC | 0.67 |
| CD.Endth/Lymph.CCL21.LYVE1 | FXYD6 | 1.82 | CD.Goblet.ITLN1.CLCA1 | FABP2 | 2.01 | CD.T.CCR7.SELL | RPL19 | 0.66 |
| CD.Endth/Lymph.CCL21.LYVE1 | PCAT19 | 1.67 | CD.Goblet.ITLN1.CLCA1 | AGR3 | 1.93 | CD.T.CCR7.SELL | RPL5 | 0.66 |
| CD.Endth/Lymph.CCL21.LYVE1 | RELN | 1.61 | CD.Goblet.ITLN1.CLCA1 | HEPACAM2 | 1.85 | CD.T.CCR7.SELL | RPS16 | 0.66 |
| CD.Endth/Lymph.CCL21.LYVE1 | GPM6A | 1.31 | CD.Goblet.ITLN1.CLCA1 | SPINK1 | 1.84 | CD.T.CCR7.SELL | RPS23 | 0.65 |
| CD.Endth/Lymph.CCL21.LYVE1 | PARD6G | 1.08 | CD.Goblet.ITLN1.CLCA1 | CLDN3 | 1.82 | CD.T.CCR7.SELL | RPL11 | 0.65 |
| CD.Endth/Lymph.CCL21.LYVE1 | GPR1 | 0.68 | CD.Goblet.ITLN1.CLCA1 | SMIM22 | 1.73 | CD.T.CCR7.SELL | RPS20 | 0.65 |
| CD.Endth/Lymph.CCL21.LYVE1 | AKAP12 | 1.96 | CD.Goblet.ITLN1.CLCA1 | S100A14 | 1.70 | CD.T.CCR7.SELL | RPL10A | 0.65 |
| CD.Endth/Lymph.CCL21.PROX1 | CCL21 | 4.61 | CD.Goblet.ITLN1.CLCA1 | EPCAM | 1.70 | CD.T.CCR7.SELL | EEF1B2 | 0.64 |
| CD.Endth/Lymph.CCL21.PROX1 | PROX1 | 2.66 | CD.Goblet.ITLN1.CLCA1 | STARD10 | 1.68 | CD.T.CCR7.SELL | RPL3 | 0.64 |
| CD.Endth/Lymph.CCL21.PROX1 | MMRN1 | 2.62 | CD.Goblet.ITLN1.CLCA1 | ST6GALNAC1 | 1.63 | CD.T.CCR7.SELL | CD3D | 0.64 |
| CD.Endth/Lymph.CCL21.PROX1 | LYVE1 | 2.24 | CD.Goblet.ITLN1.CLCA1 | REP15 | 1.59 | CD.T.CCR7.SELL | EIF3E | 0.63 |
| CD.Endth/Lymph.CCL21.PROX1 | RELN | 1.71 | CD.Goblet.ITLN1.CLCA1 | SERPINA1 | 1.56 | CD.T.CCR7.SELL | GLTSCR2 | 0.63 |
| CD.Endth/Lymph.CCL21.PROX1 | FLT4 | 1.68 | CD.Goblet.ITLN1.CLCA1 | FAM3D | 1.56 | CD.T.CCR7.SELL | TRBC2 | 0.63 |
| CD.Endth/Lymph.CCL21.PROX1 | CLEC4M | 1.64 | CD.Goblet.ITLN1.CLCA1 | KLK1 | 1.30 | CD.T.CCR7.SELL | RPS18 | 0.62 |
| CD.Endth/Lymph.EFEMP1.CCL21 | CCL21 | 4.50 | CD.Goblet.ITLN1.CLCA1 | TCEA3 | 1.29 | CD.T.CCR7.SELL | SARAF | 0.61 |
| CD.Endth/Lymph.EFEMP1.CCL21 | EFEMP1 | 3.22 | CD.Goblet.ITLN1.CLCA1 | RP11- | 1.27 | CD.T.CCR7.SELL | RPL14 | 0.60 |
| | | | | 234B24.2 | | | | |
| CD.Endth/Lymph.EFEMP1.CCL21 | MMRN1 | 2.69 | CD.Goblet.ITLN1.CLCA1 | KRT20 | 1.21 | CD.T.CCR7.SELL | TOMM7 | 0.60 |
| CD.Endth/Lymph.EFEMP1.CCL21 | SDPR | 2.48 | CD.Goblet.ITLN1.CLCA1 | LRRC26 | 1.19 | CD.T.CCR7.SELL | RPS28 | 0.60 |
| CD.Endth/Lymph.EFEMP1.CCL21 | LYVE1 | 2.47 | CD.Goblet.ITLN1.CLCA1 | CAMK2N1 | 1.16 | CD.T.CCR7.SELL | RPL13A | 0.60 |
| CD.Endth/Lymph.EFEMP1.CCL21 | RAMP2 | 2.29 | CD.Goblet.ITLN1.CLCA1 | BCAS1 | 1.15 | CD.T.CCR7.SELL | RPL6 | 0.59 |
| CD.Endth/Lymph.EFEMP1.CCL21 | CLDN5 | 2.24 | CD.Goblet.ITLN1.CLCA1 | CREB3L1 | 1.09 | CD.T.CCR7.SELL | RPL23A | 0.58 |
| CD.Endth/Lymph.EFEMP1.CCL21 | ECSCR-1 | 2.17 | CD.Goblet.ITLN1.CLCA1 | FOXA3 | 1.08 | CD.T.CCR7.SELL | TPT1 | 0.58 |
| CD.Endth/Lymph.EFEMP1.CCL21 | PPFIBP1 | 1.97 | CD.Goblet.ITLN1.CLCA1 | TSPAN1 | 1.06 | CD.T.CCR7.SELL | RPL26 | 0.58 |
| CD.Endth/Lymph.EFEMP1.CCL21 | PROX1 | 1.93 | CD.Goblet.ITLN1.CLCA1 | GALNT3 | 1.04 | CD.T.CCR7.SELL | RPS5 | 0.57 |
| CD.Endth/Lymph.EFEMP1.CCL21 | ARHGAP29 | 1.93 | CD.Goblet.ITLN1.CLCA1 | ATOH1 | 1.03 | CD.T.CCR7.SELL | NACA | 0.57 |
| CD.Endth/Lymph.EFEMP1.CCL21 | PCAT19 | 1.89 | CD.Goblet.ITLN1.CLCA1 | LINC01207 | 0.98 | CD.T.CCR7.SELL | RPSA | 0.56 |
| CD.Endth/Lymph.EFEMP1.CCL21 | EGFL7 | 1.72 | CD.Goblet.ITLN1.CLCA1 | ANG | 0.98 | CD.T.CCR7.SELL | RPS17 | 0.55 |
| CD.Endth/Lymph.EFEMP1.CCL21 | CTHRC1 | 1.68 | CD.Goblet.ITLN1.CLCA1 | CEACAM5 | 0.97 | CD.T.CCR7.SELL | RPL18 | 0.55 |
| CD.Endth/Lymph.EFEMP1.CCL21 | NRP2 | 1.62 | CD.Goblet.ITLN1.CLCA1 | SPDEF | 0.96 | CD.T.CCR7.SELL | RPS4X | 0.53 |
| CD.Endth/Lymph.EFEMP1.CCL21 | FXYD6 | 1.55 | CD.Goblet.ITLN1.CLCA1 | ANXA13 | 0.96 | CD.T.CCR7.SELL | RPL37A | 0.53 |
| CD.Endth/Lymph.EFEMP1.CCL21 | RELN | 1.43 | CD.Goblet.ITLN1.CLCA1 | TPSG1 | 0.91 | CD.T.CCR7.SELL | RPL24 | 0.53 |
| CD.Endth/Lymph.EFEMP1.CCL21 | LDB2 | 1.38 | CD.Goblet.ITLN1.CLCA1 | PLA2G10 | 0.86 | CD.T.CCR7.SELL | RPL41 | 0.52 |
| CD.Endth/Lymph.EFEMP1.CCL21 | SPHK1 | 1.30 | CD.Goblet.ITLN1.CLCA1 | C9orf152 | 0.83 | CD.T.CCR7.SELL | RPS2 | 0.52 |
| CD.Endth/Lymph.EFEMP1.CCL21 | RAI14 | 1.29 | CD.Goblet.ITLN1.CLCA1 | NEURL1 | 0.81 | CD.T.CCR7.SELL | RPL36 | 0.52 |
| CD.Endth/Lymph.EFEMP1.CCL21 | KANK3 | 1.21 | CD.Goblet.ITLN1.CLCA1 | MB | 0.78 | CD.T.CCR7.SELL | RPS7 | 0.52 |
| CD.Endth/Lymph.EFEMP1.CCL21 | TIE1 | 1.17 | CD.Goblet.ITLN1.CLCA1 | ZG16B | 0.73 | CD.T.CCR7.SELL | RPL23 | 0.52 |
| CD.Endth/Lymph.EFEMP1.CCL21 | MYCT1 | 1.16 | CD.Goblet.ITLN1.CLCA1 | UGT2B7 | 0.72 | CD.T.CCR7.SELL | PABPC1 | 0.52 |
| CD.Endth/Lymph.EFEMP1.CCL21 | OVOS2 | 1.12 | CD.Goblet.ITLN1.CLCA1 | RAP1GAP | 0.71 | CD.T.CCR7.SELL | RPL27A | 0.50 |
| CD.Endth/Lymph.EFEMP1.CCL21 | GPM6A | 1.12 | CD.Goblet.ITLN1.CLCA1 | LINC00261 | 0.63 | CD.T.CCR7.SELL | GNB2L1 | 0.49 |
| CD.Endth/Lymph.EFEMP1.CCL21 | RHOJ | 1.11 | CD.Goblet.ITLN1.CLCA1 | LYPD8 | 0.60 | CD.T.CCR7.SELL | RPL29 | 0.49 |
| CD.Endth/Lymph.EFEMP1.CCL21 | EFNA5 | 1.08 | CD.Goblet.ITLN1.CLCA1 | GALNT5 | 0.58 | CD.T.CCR7.SELL | RPL18A | 0.48 |
| CD.Endth/Lymph.EFEMP1.CCL21 | CXorf36 | 1.05 | CD.Goblet.ITLN1.CLCA1 | ABCA4 | 0.52 | CD.T.CCR7.SELL | UBA52 | 0.47 |
| CD.Endth/Lymph.EFEMP1.CCL21 | PGM5 | 1.04 | CD.Goblet.ITLN1.CLCA1 | MLPH | 0.61 | CD.T.CCR7.SELL | RPL12 | 0.46 |
| CD.Endth/Lymph.EFEMP1.CCL21 | SEMA3A | 1.02 | CD.Goblet.ITLN1.CLCA1 | KLF5 | 1.20 | CD.T.CCR7.SELL | RPS24 | 0.45 |
| CD.Endth/Lymph.EFEMP1.CCL21 | TM4SF18 | 1.01 | CD.Goblet.ITLN1.CLCA1 | RNASE1 | 1.18 | CD.T.CCR7.SELL | RPL10 | 0.44 |
| CD.Endth/Lymph.EFEMP1.CCL21 | NR2F1 | 0.94 | CD.Goblet.ITLN1.CLCA1 | GMDS | 1.40 | CD.T.CCR7.SELL | RPS15 | 0.44 |
| CD.Endth/Lymph.EFEMP1.CCL21 | ADGRG3 | 0.93 | CD.Goblet.ITLN1.CLCA1 | CLDN4 | 1.48 | CD.T.CCR7.SELL | RPS19 | 0.43 |
| CD.Endth/Lymph.EFEMP1.CCL21 | ROBO4 | 0.92 | CD.Goblet.ITLN1.CLCA1 | KRT19 | 1.51 | CD.T.CCR7.SELL | RPL28 | 0.41 |
| CD.Endth/Lymph.EFEMP1.CCL21 | FLRT2 | 0.91 | CD.Goblet.ITLN1.CLCA1 | SCNN1A | 0.60 | CD.T.CCR7.SELL | RPS9 | 0.41 |
| CD.Endth/Lymph.EFEMP1.CCL21 | PDE2A | 0.91 | CD.Goblet.ITLN1.CLCA1 | GALNT8 | 0.40 | CD.T.CCR7.SELL | RPL7A | 0.41 |
| CD.Endth/Lymph.EFEMP1.CCL21 | BMX | 0.90 | CD.Goblet.ITLN1.CLCA1 | FRZB | 0.86 | CD.T.CCR7.SELL | RPL35 | 0.41 |
| CD.Endth/Lymph.EFEMP1.CCL21 | SEMA3D | 0.83 | CD.Goblet.ITLN1.CLCA1 | SLC12A2 | 1.46 | CD.T.CCR7.SELL | MALAT1 | 0.40 |
| CD.Endth/Lymph.EFEMP1.CCL21 | RASSF9 | 0.83 | CD.Goblet.ITLN1.CLCA1 | ENTPD8 | 0.54 | CD.T.CCR7.SELL | FAU | 0.40 |
| CD.Endth/Lymph.EFEMP1.CCL21 | PARD6G | 0.78 | CD.Goblet.ITLN1.CLCA1 | PDZK1IP1 | 0.93 | CD.T.CCR7.SELL | RPL15 | 0.39 |
| CD.Endth/Lymph.EFEMP1.CCL21 | FLT4 | 0.77 | CD.Goblet.ITLN1.CLCA1.1 | ITLN1 | 2.56 | CD.T.CCR7.SELL | RPL8 | 0.34 |
| CD.Endth/Lymph.EFEMP1.CCL21 | AFAP1L1 | 0.66 | CD.Goblet.ITLN1.CLCA1.1 | CLCA1 | 3.05 | CD.T.CCR7.SELL | EEF1D | 0.43 |
| CD.Endth/Lymph.EFEMP1.CCL21 | RP11- | 0.63 | CD.Goblet.REG4.SPINK4 | SPINK4 | 3.91 | CD.T.CCR7.SELL | NPM1 | 0.50 |
| | 536018.1 | | | | | | | |
| CD.Endth/Lymph.EFEMP1.CCL21 | PALM | 0.61 | CD.Goblet.REG4.SPINK4 | CLCA1 | 3.27 | CD.T.CCR7.SELL | TRBC1 | 0.62 |
| CD.Endth/Lymph.EFEMP1.CCL21 | TBX1 | 0.56 | CD.Goblet.REG4.SPINK4 | REG4 | 3.15 | CD.T.CD8B.TRGC2 | CCL5 | 2.26 |
| CD.Endth/Lymph.EFEMP1.CCL21 | PIEZO2 | 0.55 | CD.Goblet.REG4.SPINK4 | FCGBP | 2.63 | CD.T.CD8B.TRGC2 | CD8B | 1.61 |
| CD.Endth/Lymph.EFEMP1.CCL21 | ART4 | 0.49 | CD.Goblet.REG4.SPINK4 | ITLN1 | 2.57 | CD.T.CD8B.TRGC2 | CD8A | 1.59 |
| CD.Endth/Lymph.EFEMP1.CCL21 | RP13-103911.2 | 0.48 | CD.Goblet.REG4.SPINK4 | RETNLB | 1.68 | CD.T.CD8B.TRGC2 | AC092580.4 | 1.27 |
| CD.Endth/Lymph.EFEMP1.CCL21 | RP11- | 0.38 | CD.Goblet.REG4.SPINK4 | ST6GALNAC1 | 1.64 | CD.T.CD8B.TRGC2 | TRGC2 | 1.25 |
| | 322E11.5 | | | | | | | |
| CD.Endth/Lymph.EFEMP1.CCL21 | CHRDL1 | 0.38 | CD.Goblet.REG4.SPINK4 | HEPACAM2 | 1.16 | CD.T.CD8B.TRGC2 | TNFAIP3 | 1.17 |
| CD.Endth/Lymph.EFEMP1.CCL21 | PLIN5 | 0.73 | CD.Goblet.REG4.SPINK4 | LINC00261 | 0.87 | CD.T.CD8B.TRGC2 | IL7R | 1.11 |
| CD.Endth/Lymph.EFEMP1.CCL21 | PRSS23 | 1.88 | CD.Goblet.REG4.SPINK4 | REP15 | 0.97 | CD.T.CD8B.TRGC2 | CD3E | 1.07 |
| CD.Endth/Lymph.EFEMP1.CCL21 | RBP1 | 2.48 | CD.Goblet.RETNLB.ITLN1 | CLCA1 | 3.34 | CD.T.CD8B.TRGC2 | ZFP36L2 | 1.06 |
| CD.Endth/Lymph.EFEMP1.CCL21 | FN1 | 1.37 | CD.Goblet.RETNLB.ITLN1 | ITLN1 | 2.98 | CD.T.CD8B.TRGC2 | TRAC | 1.04 |
| CD.Endth/Lymph.EFEMP1.LYVE1 | CCL21 | 4.36 | CD.Goblet.RETNLB.ITLN1 | RETNLB | 2.92 | CD.T.CD8B.TRGC2 | CD3D | 1.03 |
| CD.Endth/Lymph.EFEMP1.LYVE1 | MMRN1 | 3.63 | CD.Goblet.RETNLB.ITLN1 | FCGBP | 2.52 | CD.T.CD8B.TRGC2 | CD7 | 1.03 |
| CD.Endth/Lymph.EFEMP1.LYVE1 | EFEMP1 | 3.34 | CD.Goblet.RETNLB.ITLN1 | FXYD3 | 1.71 | CD.T.CD8B.TRGC2 | CD3G | 1.00 |
| CD.Endth/Lymph.EFEMP1.LYVE1 | LYVE1 | 3.05 | CD.Goblet.RETNLB.ITLN1 | REP15 | 1.19 | CD.T.CD8B.TRGC2 | ANXA1 | 0.99 |
| CD.Endth/Lymph.EFEMP1.LYVE1 | SDPR | 2.55 | CD.Goblet.RETNLB.ITLN1 | HEPACAM2 | 0.95 | CD.T.CD8B.TRGC2 | KLRB1 | 0.97 |
| CD.Endth/Lymph.EFEMP1.LYVE1 | PROX1 | 2.38 | CD.Goblet.RETNLB.ITLN1 | SPDEF | 0.83 | CD.T.CD8B.TRGC2 | TRBC2 | 0.95 |
| CD.Endth/Lymph.EFEMP1.LYVE1 | ECSCR-1 | 2.11 | CD.Goblet.RETNLB.ITLN1 | LRRC26 | 0.79 | CD.T.CD8B.TRGC2 | NKG7 | 0.87 |
| CD.Endth/Lymph.EFEMP1.LYVE1 | RELN | 2.01 | CD.Goblet.RETNLB.ITLN1 | ATOH1 | 0.69 | CD.T.CD8B.TRGC2 | IL32 | 0.75 |
| CD.Endth/Lymph.EFEMP1.LYVE1 | GPM6A | 1.52 | CD.Goblet.RETNLB.ITLN1 | GALNT8 | 0.66 | CD.T.CD8B.TRGC2 | RPS29 | 0.68 |
| CD.Endth/Lymph.EFEMP1.LYVE1 | PLIN5 | 1.41 | CD.Goblet.RETNLB.ITLN1 | LINC00261 | 0.58 | CD.T.CD8B.TRGC2 | RPLP1 | 0.49 |
| CD.Endth/Lymph.EFEMP1.LYVE1 | ADGRG3 | 1.36 | CD.Goblet.RETNLB.ITLN1 | SPINK4 | 3.81 | CD.T.CD8B.TRGC2 | CD160 | 0.77 |
| CD.Endth/Lymph.EFEMP1.LYVE1 | SEMA3A | 1.24 | CD.Goblet.RETNLB.ITLN1 | KLK12 | 0.69 | CD.T.CD8B.TRGC2 | SYTL3 | 0.96 |
| CD.Endth/Lymph.EFEMP1.LYVE1 | EFNA5 | 1.17 | CD.Goblet.RETNLB.ITLN1 | ST6GALNAC1 | 1.13 | CD.T.CD8B.TRGC2 | CD2 | 0.78 |
| CD.Endth/Lymph.EFEMP1.LYVE1 | CLEC4M | 1.09 | CD.Goblet.S100P.FCGBP | FCGBP | 3.14 | CD.T.CD8B.TRGC2 | PABPC1 | 0.78 |
| CD.Endth/Lymph.EFEMP1.LYVE1 | FLT4 | 0.91 | CD.Goblet.S100P.FCGBP | REG4 | 2.62 | CD.T.CD8B.TRGC2 | TMSB4X | 0.48 |
| CD.Endth/Lymph.EFEMP1.LYVE1 | RP11- | 0.86 | CD.Goblet.S100P.FCGBP | CLCA1 | 2.46 | CD.T.CTLA4.IL2RA | IL32 | 1.98 |
| | 322E11.5 | | | | | | | |
| CD.Endth/Lymph.EFEMP1.LYVE1 | TBX1 | 0.61 | CD.Goblet.S100P.FCGBP | S100P | 1.85 | CD.T.CTLA4.IL2RA | CTLA4 | 1.89 |
| CD.Endth/Lymph.EFEMP1.LYVE1 | ARHGAP29 | 2.07 | CD.Goblet.S100P.FCGBP | CDC42EP5 | 1.51 | CD.T.CTLA4.IL2RA | TIGIT | 1.67 |
| CD.Endth/Lymph.EFEMP1.LYVE1 | EGFL7 | 2.08 | CD.Goblet.S100P.FCGBP | CEACAM5 | 1.44 | CD.T.CTLA4.IL2RA | AC133644.2 | 1.56 |
| CD.Endth/Lymph.EFEMP1.LYVE1 | EMCN | 1.80 | CD.Goblet.S100P.FCGBP | TFF1 | 1.39 | CD.T.CTLA4.IL2RA | RTKN2 | 1.55 |
| CD.Endth/Lymph.LY6H.CCL21 | CCL21 | 4.26 | CD.Goblet.S100P.FCGBP | TSPAN1 | 1.24 | CD.T.CTLA4.IL2RA | AC017002.1 | 1.24 |
| CD.Endth/Lymph.LY6H.CCL21 | TFPI | 3.15 | CD.Goblet.S100P.FCGBP | ITLN1 | 1.19 | CD.T.CTLA4.IL2RA | B2M | 0.99 |
| CD.Endth/Lymph.LY6H.CCL21 | LYVE1 | 2.80 | CD.Goblet.S100P.FCGBP | REP15 | 1.13 | CD.T.GNLY.CSF2 | GNLY | 3.11 |
| CD.Endth/Lymph.LY6H.CCL21 | SDPR | 2.54 | CD.Goblet.S100P.FCGBP | SLC44A4 | 1.05 | CD.T.GNLY.CSF2 | GZMB | 2.66 |
| CD.Endth/Lymph.LY6H.CCL21 | EFEMP1 | 2.36 | CD.Goblet.S100P.FCGBP | ST6GALNAC1 | 1.04 | CD.T.GNLY.CSF2 | GZMA | 2.29 |
| CD.Endth/Lymph.LY6H.CCL21 | FXYD6 | 2.28 | CD.Goblet.S100P.FCGBP | CREB3L1 | 0.83 | CD.T.GNLY.CTSW | GNLY | 3.04 |
| CD.Endth/Lymph.LY6H.CCL21 | MMRN1 | 2.24 | CD.Goblet.S100P.FCGBP | SPDEF | 0.73 | CD.T.GNLY.CTSW | KLRC2 | 1.59 |
| CD.Endth/Lymph.LY6H.CCL21 | ECSCR-1 | 2.19 | CD.Goblet.S100P.FCGBP | BCAS1 | 0.65 | CD.T.GNLY.CTSW | ZNF683 | 1.18 |
| CD.Endth/Lymph.LY6H.CCL21 | CLDN5 | 2.04 | CD.Goblet.S100P.FCGBP | GCNT3 | 0.63 | CD.T.GNLY.GZMH | GZMH | 2.86 |
| CD.Endth/Lymph.LY6H.CCL21 | PRSS23 | 2.01 | CD.Goblet.S100P.FCGBP | RP11- | 0.49 | CD.T.GNLY.GZMH | FGFBP2 | 1.94 |
| | | | | 234B24.2 | | | | |
| CD.Endth/Lymph.LY6H.CCL21 | PPFIBP1 | 1.97 | CD.Goblet.S100P.FCGBP | MT1H | 2.31 | CD.T.GNLY.HOPX | GNLY | 3.09 |
| CD.Endth/Lymph.LY6H.CCL21 | PROX1 | 1.97 | CD.Goblet.S100P.FCGBP | MLPH | 0.71 | CD.T.GNLY.IFNG | GNLY | 3.26 |
| CD.Endth/Lymph.LY6H.CCL21 | PCAT19 | 1.88 | CD.Goblet.S100P.FCGBP | LYPD8 | 0.41 | CD.T.GNLY.IGLC2 | GNLY | 2.80 |
| CD.Endth/Lymph.LY6H.CCL21 | EGFL7 | 1.62 | CD.Goblet.S100P.FCGBP | CA12 | 0.40 | CD.T.GNLY.MT-CO2 | GNLY | 3.34 |
| CD.Endth/Lymph.LY6H.CCL21 | TIE1 | 1.44 | CD.Goblet.S100P.FCGBP | S100A14 | 1.57 | CD.T.HSPA1A.HSP90AA1 | HSP90AA1 | 1.95 |
| CD.Endth/Lymph.LY6H.CCL21 | KANK3 | 1.42 | CD.Goblet.S100P.FCGBP | HEPACAM2 | 0.57 | CD.T.HSPA1A.HSP90AA1 | IL7R | 1.89 |
| CD.Endth/Lymph.LY6H.CCL21 | CTHRC1 | 1.40 | CD.Goblet.S100P.FCGBP | CAMK2N1 | 1.05 | CD.T.IFI44L.PTGER4 | IL7R | 1.43 |
| CD.Endth/Lymph.LY6H.CCL21 | CLEC4M | 1.38 | CD.Goblet.S100P.FCGBP | MUC1 | 1.02 | CD.T.IFI44L.PTGER4 | TNFAIP3 | 1.35 |
| CD.Endth/Lymph.LY6H.CCL21 | RELN | 1.26 | CD.Goblet.S100P.FCGBP | HSD11B2 | 0.75 | CD.T.IFI44L.PTGER4 | KLRB1 | 1.33 |
| CD.Endth/Lymph.LY6H.CCL21 | NR2F1 | 1.26 | CD.Goblet.S100P.TFF1 | SPINK4 | 3.88 | CD.T.IFI44L.PTGER4 | CD69 | 1.29 |
| CD.Endth/Lymph.LY6H.CCL21 | LOX | 1.24 | CD.Goblet.S100P.TFF1 | REG4 | 3.81 | CD.T.IFI44L.PTGER4 | ANXA1 | 1.27 |
| CD.Endth/Lymph.LY6H.CCL21 | ADGRG3 | 1.22 | CD.Goblet.S100P.TFF1 | FCGBP | 3.78 | CD.T.IFI44L.PTGER4 | TSC22D3 | 1.14 |
| CD.Endth/Lymph.LY6H.CCL21 | EFNA5 | 1.16 | CD.Goblet.S100P.TFF1 | CLCA1 | 3.46 | CD.T.IFI44L.PTGER4 | TRAC | 1.11 |
| CD.Endth/Lymph.LY6H.CCL21 | PLIN5 | 1.14 | CD.Goblet.S100P.TFF1 | MT1H | 2.79 | CD.T.IFI44L.PTGER4 | CD3D | 1.10 |
| CD.Endth/Lymph.LY6H.CCL21 | FLT4 | 1.13 | CD.Goblet.S100P.TFF1 | S100P | 2.62 | CD.T.IFI44L.PTGER4 | CD2 | 1.07 |
| CD.Endth/Lymph.LY6H.CCL21 | GPM6A | 1.06 | CD.Goblet.S100P.TFF1 | TFF1 | 2.41 | CD.T.IFI44L.PTGER4 | CD3E | 1.04 |
| CD.Endth/Lymph.LY6H.CCL21 | SLC24A1 | 1.02 | CD.Goblet.S100P.TFF1 | FXYD3 | 2.30 | CD.T.KLRB1.LTF | KLRB1 | 1.88 |
| CD.Endth/Lymph.LY6H.CCL21 | PDE2A | 0.97 | CD.Goblet.S100P.TFF1 | ITLN1 | 2.04 | CD.T.KLRB1.LTF | AC092580.4 | 1.34 |
| CD.Endth/Lymph.LY6H.CCL21 | C6orf141 | 0.93 | CD.Goblet.S100P.TFF1 | S100A14 | 1.88 | CD.T.KLRB1.LTF | IL7R | 1.26 |
| CD.Endth/Lymph.LY6H.CCL21 | SCN3B | 0.74 | CD.Goblet.S100P.TFF1 | AGR3 | 1.78 | CD.T.KLRB1.LTF | S100A4 | 1.18 |
| CD.Endth/Lymph.LY6H.CCL21 | PARD6G | 0.73 | CD.Goblet.S100P.TFF1 | MT1M | 1.77 | CD.T.KLRB1.LTF | TRAC | 1.06 |
| CD.Endth/Lymph.LY6H.CCL21 | THBS4 | 0.60 | CD.Goblet.S100P.TFF1 | TSPAN1 | 1.62 | CD.T.KLRB1.LTF | CD2 | 1.03 |
| CD.Endth/Lymph.LY6H.CCL21 | GPR1 | 0.60 | CD.Goblet.S100P.TFF1 | RETNLB | 1.62 | CD.T.KLRB1.LTF | CD3D | 1.01 |
| CD.Endth/Lymph.LY6H.CCL21 | CTB-50L17.14 | 0.55 | CD.Goblet.S100P.TFF1 | CDC42EP5 | 1.61 | CD.T.KLRB1.LTF | RORA | 1.01 |
| CD.Endth/Lymph.LY6H.CCL21 | RP11- | 0.53 | CD.Goblet.S100P.TFF1 | REP15 | 1.59 | CD.T.KLRB1.LTF | ANXA1 | 0.98 |
| | 136K7.2 | | | | | | | |
| CD.Endth/Lymph.LY6H.CCL21 | RP11- | 0.49 | CD.Goblet.S100P.TFF1 | FAM3D | 1.58 | CD.T.KLRB1.LTF | TNFAIP3 | 0.93 |
| | 322E11.5 | | | | | | | |
| CD.Endth/Lymph.LY6H.CCL21 | PDPN | 1.18 | CD.Goblet.S100P.TFF1 | CAMK2N1 | 1.54 | CD.T.KLRB1.LTF | CD3E | 0.85 |
| CD.Endth/Mural.HIGD1B.NDUFA4L2 | RGS5 | 3.69 | CD.Goblet.S100P.TFF1 | ST6GALNAC1 | 1.54 | CD.T.KLRB1.LTF | RPS29 | 0.85 |
| CD.Endth/Mural.HIGD1B.NDUFA4L2 | PLVAP | 3.01 | CD.Goblet.S100P.TFF1 | CEACAM5 | 1.52 | CD.T.KLRB1.LTF | IL32 | 0.73 |
| CD.Endth/Mural.HIGD1B.NDUFA4L2 | SPARCL1 | 2.79 | CD.Goblet.S100P.TFF1 | KRT20 | 1.52 | CD.T.KLRB1.LTF | PABPC1 | 0.73 |
| CD.Endth/Mural.HIGD1B.NDUFA4L2 | A2M | 2.56 | CD.Goblet.S100P.TFF1 | TCEA3 | 1.38 | CD.T.KLRB1.LTF | RPLP1 | 0.71 |
| CD.Endth/Mural.HIGD1B.NDUFA4L2 | CD36 | 2.43 | CD.Goblet.S100P.TFF1 | NPDC1 | 1.32 | CD.T.KLRB1.LTF | RPL39 | 0.61 |
| CD.Endth/Mural.HIGD1B.NDUFA4L2 | CAV1 | 2.42 | CD.Goblet.S100P.TFF1 | RP11- | 1.31 | CD.T.KLRB1.LTF | RPS27 | 0.56 |
| | | | | 462G2.1 | | | | |
| CD.Endth/Mural.HIGD1B.NDUFA4L2 | SPARC | 2.32 | CD.Goblet.S100P.TFF1 | CREB3L1 | 1.21 | CD.T.KLRB1.LTF | RPS4X | 0.55 |
| CD.Endth/Mural.HIGD1B.NDUFA4L2 | GNG11 | 2.31 | CD.Goblet.S100P.TFF1 | HEPACAM2 | 1.20 | CD.T.KLRB1.LTF | TMSB4X | 0.53 |
| CD.Endth/Mural.HIGD1B.NDUFA4L2 | RAMP2 | 2.30 | CD.Goblet.S100P.TFF1 | BCAS1 | 1.13 | CD.T.KLRB1.LTF | RPL41 | 0.47 |
| CD.Endth/Mural.HIGD1B.NDUFA4L2 | SLC9A3R2 | 2.25 | CD.Goblet.S100P.TFF1 | FOXA3 | 1.09 | CD.T.KLRB1.LTF | RPL34 | 0.45 |
| CD.Endth/Mural.HIGD1B.NDUFA4L2 | IGFBP4 | 2.17 | CD.Goblet.S100P.TFF1 | SPDEF | 1.03 | CD.T.KLRB1.LTF | CD3G | 0.87 |
| CD.Endth/Mural.HIGD1B.NDUFA4L2 | CALD1 | 2.15 | CD.Goblet.S100P.TFF1 | MLPH | 1.00 | CD.T.KLRB1.LTF | EEF1A1 | 0.45 |
| CD.Endth/Mural.HIGD1B.NDUFA4L2 | MGP | 2.12 | CD.Goblet.S100P.TFF1 | RP11- | 0.96 | CD.T.KLRB1.LTF | RPS28 | 0.45 |
| | | | | 234B24.2 | | | | |
| CD.Endth/Mural.HIGD1B.NDUFA4L2 | RAMP3 | 2.10 | CD.Goblet.S100P.TFF1 | ANXA13 | 0.93 | CD.T.KLRB1.LTF | GIMAP7 | 0.89 |
| CD.Endth/Mural.HIGD1B.NDUFA4L2 | CLDN5 | 2.09 | CD.Goblet.S100P.TFF1 | TPSG1 | 0.89 | CD.T.KLRB1.LTF | LTB | 0.78 |
| CD.Endth/Mural.HIGD1B.NDUFA4L2 | PRSS23 | 1.97 | CD.Goblet.S100P.TFF1 | LYPD8 | 0.85 | CD.T.MAF.CTLA4 | KLRB1 | 2.11 |
| CD.Endth/Mural.HIGD1B.NDUFA4L2 | TM4SF1 | 1.95 | CD.Goblet.S100P.TFF1 | PLA2G10 | 0.82 | CD.T.MKI67.CD38 | HBA1 | 1.89 |
| CD.Endth/Mural.HIGD1B.NDUFA4L2 | COL15A1 | 1.92 | CD.Goblet.S100P.TFF1 | CEACAM6 | 0.78 | CD.T.MKI67.FOXP3 | KIAA0101 | 1.85 |
| CD.Endth/Mural.HIGD1B.NDUFA4L2 | NDUFA4L2 | 1.91 | CD.Goblet.S100P.TFF1 | LRRC26 | 0.77 | CD.T.MKI67.FOXP3 | TOP2A | 1.75 |
| CD.Endth/Mural.HIGD1B.NDUFA4L2 | FLT1 | 1.90 | CD.Goblet.S100P.TFF1 | GALNT5 | 0.70 | CD.T.MKI67.FOXP3 | CENPF | 1.75 |
| CD.Endth/Mural.HIGD1B.NDUFA4L2 | EPAS1 | 1.89 | CD.Goblet.S100P.TFF1 | ATOH1 | 0.69 | CD.T.MKI67.FOXP3 | MKI67 | 1.71 |
| CD.Endth/Mural.HIGD1B.NDUFA4L2 | CRIP2 | 1.89 | CD.Goblet.S100P.TFF1 | ABCA4 | 0.67 | CD.T.MKI67.FOXP3 | NUSAP1 | 1.66 |
| CD.Endth/Mural.HIGD1B.NDUFA4L2 | ARHGAP29 | 1.78 | CD.Goblet.S100P.TFF1 | LINC00261 | 0.61 | CD.T.MKI67.FOXP3 | BIRC5 | 1.35 |
| CD.Endth/Mural.HIGD1B.NDUFA4L2 | TMEM88 | 1.76 | CD.Goblet.S100P.TFF1 | RAP1GAP | 0.61 | CD.T.MKI67.FOXP3 | TYMS | 1.24 |
| CD.Endth/Mural.HIGD1B.NDUFA4L2 | PTRF | 1.74 | CD.Goblet.S100P.TFF1 | BEST2 | 0.56 | CD.T.MKI67.FOXP3 | CEP55 | 0.95 |
| CD.Endth/Mural.HIGD1B.NDUFA4L2 | MYL9 | 1.74 | CD.Goblet.S100P.TFF1 | TMEM61 | 0.55 | CD.T.MKI67.FOXP3 | FOXP3 | 0.77 |
| CD.Endth/Mural.HIGD1B.NDUFA4L2 | NFIB | 1.69 | CD.Goblet.S100P.TFF1 | GPR153 | 0.55 | CD.T.MKI67.FOXP3 | CTLA4 | 1.35 |
| CD.Endth/Mural.HIGD1B.NDUFA4L2 | SOD3 | 1.67 | CD.Goblet.S100P.TFF1 | B3GNT6 | 0.55 | CD.T.MKI67.FOXP3 | IL2RA | 1.46 |
| CD.Endth/Mural.HIGD1B.NDUFA4L2 | TIMP3 | 1.67 | CD.Goblet.S100P.TFF1 | GALNT8 | 0.50 | CD.T.MKI67.FOXP3 | TPX2 | 1.01 |
| CD.Endth/Mural.HIGD1B.NDUFA4L2 | HIGD1B | 1.64 | CD.Goblet.S100P.TFF1 | SCNN1A | 0.67 | CD.T.MKI67.FOXP3 | TK1 | 1.22 |
| CD.Endth/Mural.HIGD1B.NDUFA4L2 | ADGRL4 | 1.64 | CD.Goblet.S100P.TFF1 | LINC01207 | 0.96 | CD.T.MKI67.FOXP3 | NUF2 | 0.97 |
| CD.Endth/Mural.HIGD1B.NDUFA4L2 | HSPG2 | 1.63 | CD.Goblet.S100P.TFF1 | CAPN9 | 0.28 | CD.T.MKI67.IFNG | KIAA0101 | 1.78 |
| CD.Endth/Mural.HIGD1B.NDUFA4L2 | RNASE1 | 1.62 | CD.Goblet.S100P.TFF1 | FABP2 | 1.44 | CD.T.MKI67.IFNG | TYMS | 1.46 |
| CD.Endth/Mural.HIGD1B.NDUFA4L2 | PODXL | 1.60 | CD.Goblet.S100P.TFF1 | C9orf152 | 0.76 | CD.T.MKI67.IFNG | IL26 | 0.79 |
| CD.Endth/Mural.HIGD1B.NDUFA4L2 | COL4A1 | 1.58 | CD.Goblet.TFF1.TPSG1 | REG4 | 4.08 | CD.T.MKI67.IFNG | IFNG | 2.07 |
| CD.Endth/Mural.HIGD1B.NDUFA4L2 | ADGRF5 | 1.58 | CD.Goblet.TFF1.TPSG1 | CLCA1 | 3.20 | CD.T.MKI67.IFNG | RRM2 | 1.60 |
| CD.Endth/Mural.HIGD1B.NDUFA4L2 | FRZB | 1.57 | CD.Goblet.TFF1.TPSG1 | FCGBP | 2.98 | CD.T.MKI67.IFNG | MKI67 | 1.30 |
| CD.Endth/Mural.HIGD1B.NDUFA4L2 | EGFL7 | 1.56 | CD.Goblet.TFF1.TPSG1 | TFF1 | 2.71 | CD.T.MKI67.IL22 | TOP2A | 2.34 |
| CD.Endth/Mural.HIGD1B.NDUFA4L2 | ENG | 1.56 | CD.Goblet.TFF1.TPSG1 | ITLN1 | 2.40 | CD.T.MKI67.IL22 | NUSAP1 | 2.00 |
| CD.Endth/Mural.HIGD1B.NDUFA4L2 | ESAM | 1.55 | CD.Goblet.TFF1.TPSG1 | FXYD3 | 2.36 | CD.T.MKI67.IL22 | CENPF | 1.97 |
| CD.Endth/Mural.HIGD1B.NDUFA4L2 | EMCN | 1.55 | CD.Goblet.TFF1.TPSG1 | CEACAM5 | 1.99 | CD.T.MKI67.IL22 | MKI67 | 1.96 |
| CD.Endth/Mural.HIGD1B.NDUFA4L2 | VWA1 | 1.53 | CD.Goblet.TFF1.TPSG1 | S100A14 | 1.95 | CD.T.MKI67.IL22 | UBE2C | 1.92 |
| CD.Endth/Mural.HIGD1B.NDUFA4L2 | FAM167B | 1.52 | CD.Goblet.TFF1.TPSG1 | ZG16 | 1.89 | CD.T.MKI67.IL22 | KIAA0101 | 1.84 |
| CD.Endth/Mural.HIGD1B.NDUFA4L2 | SDPR | 1.51 | CD.Goblet.TFF1.TPSG1 | S100P | 1.63 | CD.T.MKI67.IL22 | RRM2 | 1.75 |
| CD.Endth/Mural.HIGD1B.NDUFA4L2 | COL4A2 | 1.51 | CD.Goblet.TFF1.TPSG1 | ST6GALNAC1 | 1.53 | CD.T.MKI67.IL22 | ASPM | 1.67 |
| CD.Endth/Mural.HIGD1B.NDUFA4L2 | JAM2 | 1.50 | CD.Goblet.TFF1.TPSG1 | KRT20 | 1.42 | CD.T.MKI67.IL22 | CDK1 | 1.62 |
| CD.Endth/Mural.HIGD1B.NDUFA4L2 | PLPP3 | 1.50 | CD.Goblet.TFF1.TPSG1 | FAM3D | 1.42 | CD.T.MKI67.IL22 | TYMS | 1.51 |
| CD.Endth/Mural.HIGD1B.NDUFA4L2 | TM4SF18 | 1.50 | CD.Goblet.TFF1.TPSG1 | TSPAN1 | 1.37 | CD.T.MKI67.IL22 | BIRC5 | 1.40 |
| CD.Endth/Mural.HIGD1B.NDUFA4L2 | VWF | 1.48 | CD.Goblet.TFF1.TPSG1 | REP15 | 1.36 | CD.T.MKI67.IL22 | CCNB2 | 1.21 |
| CD.Endth/Mural.HIGD1B.NDUFA4L2 | RBP7 | 1.47 | CD.Goblet.TFF1.TPSG1 | PPIC | 1.21 | CD.T.MKI67.IL22 | DLGAP5 | 1.20 |
| CD.Endth/Mural.HIGD1B.NDUFA4L2 | LIFR | 1.46 | CD.Goblet.TFF1.TPSG1 | NPDC1 | 1.21 | CD.T.MKI67.IL22 | TPX2 | 1.20 |
| CD.Endth/Mural.HIGD1B.NDUFA4L2 | COX412 | 1.46 | CD.Goblet.TFF1.TPSG1 | FAM3B | 1.18 | CD.T.MKI67.IL22 | ZWINT | 1.20 |
| CD.Endth/Mural.HIGD1B.NDUFA4L2 | NR2F2 | 1.46 | CD.Goblet.TFF1.TPSG1 | TPSG1 | 1.14 | CD.T.MKI67.IL22 | CCNA2 | 1.19 |
| CD.Endth/Mural.HIGD1B.NDUFA4L2 | NPDC1 | 1.42 | CD.Goblet.TFF1.TPSG1 | LINC00261 | 1.06 | CD.T.MKI67.IL22 | CDKN3 | 1.19 |
| CD.Endth/Mural.HIGD1B.NDUFA4L2 | FABP4 | 1.41 | CD.Goblet.TFF1.TPSG1 | LINC01207 | 1.05 | CD.T.MKI67.IL22 | NUF2 | 1.16 |
| CD.Endth/Mural.HIGD1B.NDUFA4L2 | TINAGL1 | 1.40 | CD.Goblet.TFF1.TPSG1 | BCAS1 | 1.05 | CD.T.MKI67.IL22 | GTSE1 | 1.15 |
| CD.Endth/Mural.HIGD1B.NDUFA4L2 | CD93 | 1.39 | CD.Goblet.TFF1.TPSG1 | ANG | 1.03 | CD.T.MKI67.IL22 | HMMR | 1.12 |
| CD.Endth/Mural.HIGD1B.NDUFA4L2 | CLEC14A | 1.37 | CD.Goblet.TFF1.TPSG1 | FHL2 | 1.00 | CD.T.MKI67.IL22 | AURKB | 1.12 |
| CD.Endth/Mural.HIGD1B.NDUFA4L2 | CYYR1 | 1.36 | CD.Goblet.TFF1.TPSG1 | CREB3L1 | 0.99 | CD.T.MKI67.IL22 | CENPA | 1.06 |
| CD.Endth/Mural.HIGD1B.NDUFA4L2 | CD34 | 1.35 | CD.Goblet.TFF1.TPSG1 | SPDEF | 0.97 | CD.T.MKI67.IL22 | NDC80 | 1.05 |
| CD.Endth/Mural.HIGD1B.NDUFA4L2 | CAV2 | 1.34 | CD.Goblet.TFF1.TPSG1 | MUC1 | 0.97 | CD.T.MKI67.IL22 | KIF23 | 1.03 |
| CD.Endth/Mural.HIGD1B.NDUFA4L2 | THBD | 1.32 | CD.Goblet.TFF1.TPSG1 | HEPACAM2 | 0.95 | CD.T.MKI67.IL22 | CEP55 | 1.01 |
| CD.Endth/Mural.HIGD1B.NDUFA4L2 | PLAT | 1.32 | CD.Goblet.TFF1.TPSG1 | MUC13 | 0.85 | CD.T.MKI67.IL22 | NCAPG | 0.94 |
| CD.Endth/Mural.HIGD1B.NDUFA4L2 | SLCO2A1 | 1.32 | CD.Goblet.TFF1.TPSG1 | MLPH | 0.81 | CD.T.MKI67.IL22 | IL26 | 0.93 |
| CD.Endth/Mural.HIGD1B.NDUFA4L2 | LHFP | 1.31 | CD.Goblet.TFF1.TPSG1 | FAM177B | 0.81 | CD.T.MKI67.IL22 | BUB1 | 0.90 |
| CD.Endth/Mural.HIGD1B.NDUFA4L2 | CALCRL | 1.26 | CD.Goblet.TFF1.TPSG1 | URAD | 0.80 | CD.T.MKI67.IL22 | HJURP | 0.86 |
| CD.Endth/Mural.HIGD1B.NDUFA4L2 | STEAP4 | 1.26 | CD.Goblet.TFF1.TPSG1 | PLA2G10 | 0.80 | CD.T.MKI67.IL22 | SPC25 | 0.86 |
| CD.Endth/Mural.HIGD1B.NDUFA4L2 | SYNPO | 1.24 | CD.Goblet.TFF1.TPSG1 | MAL2 | 0.79 | CD.T.MKI67.IL22 | KIF2C | 0.84 |
| CD.Endth/Mural.HIGD1B.NDUFA4L2 | NOTCH3 | 1.24 | CD.Goblet.TFF1.TPSG1 | CDH1 | 0.78 | CD.T.MKI67.IL22 | CKAP2L | 0.66 |
| CD.Endth/Mural.HIGD1B.NDUFA4L2 | PRKCDBP | 1.23 | CD.Goblet.TFF1.TPSG1 | LRRC26 | 0.74 | CD.T.MKI67.IL22 | TTK | 0.71 |
| CD.Endth/Mural.HIGD1B.NDUFA4L2 | MYO1B | 1.23 | CD.Goblet.TFF1.TPSG1 | ZG16B | 0.73 | CD.T.MKI67.IL22 | CDC20 | 0.98 |
| CD.Endth/Mural.HIGD1B.NDUFA4L2 | BCAM | 1.20 | CD.Goblet.TFF1.TPSG1 | FOXA3 | 0.72 | CD.T.MKI67.IL22 | KIF14 | 0.66 |
| CD.Endth/Mural.HIGD1B.NDUFA4L2 | COX7A1 | 1.19 | CD.Goblet.TFF1.TPSG1 | RP11- | 0.67 | CD.T.MKI67.IL22 | KIF11 | 0.92 |
| | | | | 234B24.2 | | | | |
| CD.Endth/Mural.HIGD1B.NDUFA4L2 | CPE | 1.18 | CD.Goblet.TFF1.TPSG1 | ARFGEF3 | 0.64 | CD.T.MKI67.IL22 | CENPE | 0.99 |
| CD.Endth/Mural.HIGD1B.NDUFA4L2 | HEG1 | 1.17 | CD.Goblet.TFF1.TPSG1 | GALNT5 | 0.60 | CD.T.MKI67.IL22 | PRC1 | 0.88 |
| CD.Endth/Mural.HIGD1B.NDUFA4L2 | HYAL2 | 1.17 | CD.Goblet.TFF1.TPSG1 | SDCBP2 | 0.56 | CD.T.MKI67.IL22 | MAD2L1 | 1.33 |
| CD.Endth/Mural.HIGD1B.NDUFA4L2 | NRP1 | 1.17 | CD.Goblet.TFF1.TPSG1 | B3GNT6 | 0.55 | CD.T.MKI67.IL22 | PLK1 | 0.79 |
| CD.Endth/Mural.HIGD1B.NDUFA4L2 | KDR | 1.15 | CD.Goblet.TFF1.TPSG1 | CEACAM6 | 0.55 | CD.T.MKI67.IL22 | CDCA8 | 0.73 |
| CD.Endth/Mural.HIGD1B.NDUFA4L2 | NQO1 | 1.14 | CD.Goblet.TFF1.TPSG1 | MBOAT2 | 0.52 | CD.T.MKI67.IL22 | CLSPN | 0.93 |
| CD.Endth/Mural.HIGD1B.NDUFA4L2 | RBP5 | 1.14 | CD.Goblet.TFF1.TPSG1 | ENTPD8 | 0.51 | CD.T.MT-CO2.MT-CO1 | MALAT1 | 1.33 |
| CD.Endth/Mural.HIGD1B.NDUFA4L2 | PCDH17 | 1.12 | CD.Goblet.TFF1.TPSG1 | SCNN1A | 0.50 | CD.T.MT-CO2.MT-CO1 | MT-CO1 | 0.78 |
| CD.Endth/Mural.HIGD1B.NDUFA4L2 | PTPRB | 1.11 | CD.Goblet.TFF1.TPSG1 | MUC4 | 0.50 | CD.T.MT-CO2.MT-CO1 | MT-ND2 | 0.99 |
| CD.Endth/Mural.HIGD1B.NDUFA4L2 | DLC1 | 1.11 | CD.Goblet.TFF1.TPSG1 | DNAJC12 | 0.49 | CD.T.MT-CO2.MT-CO1 | MT-CO2 | 0.80 |
| CD.Endth/Mural.HIGD1B.NDUFA4L2 | MMRN2 | 1.10 | CD.Goblet.TFF1.TPSG1 | CAPN8 | 0.48 | CD.T.MT-CO2.MT-CO1 | MT-CYB | 0.89 |
| CD.Endth/Mural.HIGD1B.NDUFA4L2 | PCAT19 | 1.09 | CD.Goblet.TFF1.TPSG1 | GALNT12 | 0.47 | CD.T.MT-CO2.MT-CO1 | MT-CO3 | 0.75 |
| CD.Endth/Mural.HIGD1B.NDUFA4L2 | LDB2 | 1.08 | CD.Goblet.TFF1.TPSG1 | TTC39A | 0.47 | CD.T.PDE3B.IL7R | IL7R | 1.88 |
| CD.Endth/Mural.HIGD1B.NDUFA4L2 | BGN | 1.08 | CD.Goblet.TFF1.TPSG1 | ABCA4 | 0.45 | CD.T.PDE3B.IL7R | CCL5 | 1.63 |
| CD.Endth/Mural.HIGD1B.NDUFA4L2 | CDH5 | 1.07 | CD.Goblet.TFF1.TPSG1 | RAP1GAP | 0.44 | CD.T.PDE3B.IL7R | CD8A | 1.36 |
| CD.Endth/Mural.HIGD1B.NDUFA4L2 | ECSCR-1 | 1.05 | CD.Goblet.TFF1.TPSG1 | FFAR4 | 0.44 | CD.T.PDE3B.IL7R | TPT1 | 1.12 |
| CD.Endth/Mural.HIGD1B.NDUFA4L2 | ASPN | 1.04 | CD.Goblet.TFF1.TPSG1 | GALNT8 | 0.36 | CD.T.PDE3B.IL7R | PDE3B | 1.32 |
| CD.Endth/Mural.HIGD1B.NDUFA4L2 | UACA | 1.04 | CD.Goblet.TFF1.TPSG1 | MUC5B | 0.31 | CD.T.TIGIT.CTLA4 | TIGIT | 1.90 |
| CD.Endth/Mural.HIGD1B.NDUFA4L2 | NOTCH4 | 1.04 | CD.Goblet.TFF1.TPSG1 | STXBP5-AS1 | 0.30 | CD.T.TIGIT.CTLA4 | CTLA4 | 1.48 |
| CD.Endth/Mural.HIGD1B.NDUFA4L2 | NOSTRIN | 1.04 | CD.Goblet.TFF1.TPSG1 | CGREF1 | 0.29 | CD.T.TNFRSF18.FOXP3 | TNFRSF4 | 2.82 |
| CD.Endth/Mural.HIGD1B.NDUFA4L2 | SLC14A1 | 1.03 | CD.Goblet.TFF1.TPSG1 | RBP4 | 0.28 | CD.T.TNFRSF18.FOXP3 | TNFRSF18 | 2.48 |
| CD.Endth/Mural.HIGD1B.NDUFA4L2 | PDGFRB | 1.02 | CD.Goblet.TFF1.TPSG1 | TMEM61 | 0.28 | CD.T.TNFRSF18.FOXP3 | BATF | 2.03 |
| CD.Endth/Mural.HIGD1B.NDUFA4L2 | CSRP2 | 0.98 | CD.Goblet.TFF1.TPSG1 | ERN2 | 0.48 | CD.T.TNFRSF18.FOXP3 | CTLA4 | 1.96 |
| CD.Endth/Mural.HIGD1B.NDUFA4L2 | SOX18 | 0.98 | CD.Goblet.TFF1.TPSG1 | C9orf152 | 0.54 | CD.T.TNFRSF18.FOXP3 | IL2RA | 1.80 |
| CD.Endth/Mural.HIGD1B.NDUFA4L2 | SYNPO2 | 0.98 | CD.Goblet.TFF1.TPSG1 | KCNK1 | 0.48 | CD.T.TNFRSF18.FOXP3 | TIGIT | 1.70 |
| CD.Endth/Mural.HIGD1B.NDUFA4L2 | FCN3 | 0.98 | CD.Goblet.TFF1.TPSG1 | LIPH | 0.72 | CD.T.TNFRSF18.FOXP3 | AC133644.2 | 1.67 |
| CD.Endth/Mural.HIGD1B.NDUFA4L2 | NES | 0.98 | CD.Goblet.TFF1.TPSG1 | GALNT7 | 0.86 | CD.T.TNFRSF18.FOXP3 | TNFRSF9 | 1.59 |
| CD.Endth/Mural.HIGD1B.NDUFA4L2 | TMEM204 | 0.97 | CD.Goblet.TFF1.TPSG1 | HES6 | 0.76 | CD.T.TNFRSF18.FOXP3 | IL1R2 | 1.49 |
| CD.Endth/Mural.HIGD1B.NDUFA4L2 | MCAM | 0.97 | CD.Goblet.TFF1.TPSG1 | GALE | 0.76 | CD.T.TNFRSF18.FOXP3 | CXCR6 | 1.31 |
| CD.Endth/Mural.HIGD1B.NDUFA4L2 | LIMCH1 | 0.96 | CD.Goblet.TFF1.TPSG1 | AGR3 | 1.56 | CD.T.TNFRSF18.FOXP3 | AC017002.1 | 1.16 |
| CD.Endth/Mural.HIGD1B.NDUFA4L2 | CA4 | 0.93 | CD.Goblet.TFF1.TPSG1 | B3GNT7 | 0.80 | CD.T.TNFRSF18.FOXP3 | TNIP3 | 1.14 |
| CD.Endth/Mural.HIGD1B.NDUFA4L2 | EMP1 | 0.93 | CD.Goblet.TFF1.TPSG1 | PRUNE2 | 0.44 | CD.T.TNFRSF18.FOXP3 | LAIR2 | 1.05 |
| CD.Endth/Mural.HIGD1B.NDUFA4L2 | NUAK1 | 0.92 | CD.Goblet.TFF1.TPSG1 | SGMS2 | 0.35 | CD.T.TNFRSF18.FOXP3 | FOXP3 | 1.05 |
| CD.Endth/Mural.HIGD1B.NDUFA4L2 | FAM107A | 0.92 | CD.Hirschsprung.FIGF.CPM | PDGFD | 1.39 | CD.T.TNFRSF18.FOXP3 | F5 | 0.72 |
| CD.Endth/Mural.HIGD1B.NDUFA4L2 | IL3RA | 0.92 | CD.Hirschsprung.FIGF.CPM | FAM150B | 1.39 | CD.T.TNFRSF18.FOXP3 | TBC1D4 | 1.22 |
| CD.Endth/Mural.HIGD1B.NDUFA4L2 | GPRC5B | 0.92 | CD.Hirschsprung.FIGF.CPM | MMP11 | 1.39 | CD.T.TNFRSF4.MAF | TIGIT | 1.62 |
| CD.Endth/Mural.HIGD1B.NDUFA4L2 | APLNR | 0.91 | CD.Hirschsprung.FIGF.CPM | FIGF | 1.36 | CD.T.TRDC.SOX4 | TRDC | 1.53 |
| CD.Endth/Mural.HIGD1B.NDUFA4L2 | MAP1B | 0.91 | CD.Hirschsprung.FIGF.CPM | PCSK6 | 1.02 | CD.T.TRDC.TRGC2 | TRGC2 | 1.54 |
| CD.Endth/Mural.HIGD1B.NDUFA4L2 | PLCB1 | 0.91 | CD.Hirschsprung.FIGF.CPM | PODN | 0.72 | CD.T.TRDC.TRGC2 | CCL5 | 1.49 |
| CD.Endth/Mural.HIGD1B.NDUFA4L2 | PKP4 | 0.90 | CD.Hirschsprung.FIGF.CPM | EDNRB | 2.45 | CD.T.TRDC.TRGC2 | TRDC | 1.74 |
| CD.Endth/Mural.HIGD1B.NDUFA4L2 | NOV | 0.89 | CD.Hirschsprung.FIGF.CPM | PDGFRA | 2.16 | CD.T.TRDC.TRGC2 | GZMK | 1.25 |
| CD.Endth/Mural.HIGD1B.NDUFA4L2 | C11orf96 | 0.88 | CD.Hirschsprung.FIGF.CPM | APOD | 2.28 | CD.TA.PLCG2.SOX4 | PLCG2 | 2.95 |
| CD.Endth/Mural.HIGD1B.NDUFA4L2 | S1PR1 | 0.86 | CD.Hirschsprung.FIGF.CPM | BMP4 | 1.85 | CD.TA.PLCG2.SOX4 | ELF3 | 2.27 |
| CD.Endth/Mural.HIGD1B.NDUFA4L2 | TMTC1 | 0.86 | CD.Hirschsprung.FIGF.CPM | COL12A1 | 1.26 | CD.TA.PLCG2.SOX4 | KRT18 | 1.79 |
| CD.Endth/Mural.HIGD1B.NDUFA4L2 | SOX17 | 0.84 | CD.ILC.AREG.AHR | KRT86 | 2.07 | CD.TA.PLCG2.SOX4 | DMBT1 | 1.77 |
| CD.Endth/Mural.HIGD1B.NDUFA4L2 | MYCT1 | 0.83 | CD.ILC.AREG.AHR | KRT81 | 1.89 | CD.TA.PLCG2.SOX4 | PHGR1 | 1.31 |
| CD.Endth/Mural.HIGD1B.NDUFA4L2 | FAM13C | 0.82 | CD.ILC.AREG.AHR | KIT | 1.57 | CD.TA.PLCG2.SOX4 | SOX4 | 2.01 |
| CD.Endth/Mural.HIGD1B.NDUFA4L2 | TNS2 | 0.81 | CD.ILC.AREG.AHR | TRDC | 2.24 | | | |

TABLE 1B
Expanded list of CD markers for specific subsets of Tier 1 cell types.

CD.B/DZ.HIST1H1B.MKI67
HIST1H1B, RRM2, GTSE1, CDK1, MKI67, SHCBP1, SPC25, KIF11, TOP2A, PBK, UBE2C, AURKB, NUSAP1, ESCO2, RGS13,
HIST1H2AM, HIST1H1D, HJURP, NCAPH, NCAPG, E2F8, DLGAP5, KIFC1, FBXO5, CCNA2, MAP2, TK1, BCL7A, KIAA0101,
CLSPN, CCNB2, NDC80, HIST1H2AH, MYBL1, BUB1, KIF4A, AICDA, CENPU, NEIL3, ORC6, TYMS, TUBB2B, HIST1H1E,
KATNAL1, CENPM, MYBL2, KIF15, CASC5, CENPF, BRCA1, CENPK, EZH2, ZWINT, PLK4, HIST2H2AC, GCSAM,
ARHGAP11A, DHFR, NUF2, TPX2, FAM111B, LRMP, FEN1, SMC2, RMI2, CDCA8, BIRC5, SERPINA9, BIK, ASF1B,
HIST1H2AJ, HAUS8, AC023590.1, CDKN3, NCAPG2, RAD51, PTTG1, STAG3, CDCA2, LMNB1, MND1, CDCA5, RMI1,
MED12L, ASPM, UBE2T, RPRD1B, HMMR, RRM1, CDCA7, MELK, CENPA, CDC45, KIF2C, AP000251.3, CCNB1, RP11-
231C14.7, IGSF10, CDC20, NUGGC, MXD3, CHEK1, N4BP3, BARD1, BUB1B, CENPE, UHRF1, MAD2L1, E2F1, TCL1A,
PLK1, TACC3, ATAD2, WEE1, CCDC144A, RNGTT
CD.cDC2.CD1C.AREG
MNDA, CLEC10A, MS4A6A, IDO1, IFI30, CPVL, IGSF6, CD86, CD1E, CFP, CLEC4A, P2RY13, CD207, MRC1, JAML,
FAM110A, CACNA2D3, CD1C, LST1, SPI1, HCK, FGL2, PAK1, CEACAM4, CSF2RA, PLAUR, SLC8A1, LGALS2, IL1B,
FAM26F, AIF1, GCA, SAMHD1, SGK1, KLF4, PLD4, NR4A3, FCER1A, HLA-DQB2, ITGAX, CD1D, CLIC2, NCF2, GPAT3,
FCER1G, TYROBP, PTPRE, KYNU, RNASE6, CLEC7A, C1orf162, PLSCR1, CYBB, BID, FPR1, HLA-DQA1, SCIMP, CPPED1,
HLA-DMB, PLEK, AOAH, FNIP2, LYZ, FGR, STX11, LAP3, RAB31, P2RY14, HLA-DQB1, SLC31A2, OSM, LPCAT2, TPMT,
CST3, FLT3, SMCO4, SERPINB9, PPA1, HLA-DRB1, HLA-DPB1, CCDC88A, TIMP1, NAMPT, GSN, FAM49A, C15orf48,
TNFAIP8L2, BCL2A1, UBE2E2, HLA-DMA, C10orf128, THBD, LGALS9, FPR3, INSIG1, RNF130, LITAF, WARS, HLA-DPA1,
ATG3, TYMP, PYCARD, HLA-DRA, PTGS2, IFNGR1, FAM105A, RGS18, GBP1, KCNMB1, SLAMF8, PRCP, ATP6V1B2, 1-
Mar, TIGAR, GRN, PLEKHO1, CXCL16, AREG, RAB32, THEMIS2, MS4A7, ITGB2, SPECC1, CTSZ, PHACTR1, P2RY6, AP1S2,
GK, PPT1, LCP1, PHLDA2, H2AFY, G0S2, COTL1, CD74, MGST2, GZF1, ACTB, CEBPD, MFSD1, PKIB, SERPINB1, GPX1,
MCTP1, RPS6KA4, FAM49B, VRK2, MAT2A, GPR183, LSP1, S100A11, PLAC8, C9orf72, CTSH, DSE, TBXAS1, SNX10,
SPINT2, SPATS2L, ACAA1, SNAI1, CASP1, TMSB10, ANXA5, ARPC5, LIMS1, HAVCR2, PRELID1, NDRG2, ZNF385A,
ARL5B, HIC1, ATF5, PLBD1, CAT, LY86, PPP1R14A, GBP4, PID1, IFITM3, LILRB3, SAT1, EMILIN2, SAMD9L, RP11-
1143G9.4, NAIP, FOSL2, ALDH2, CD33, MXD1, WAS, GDI2, PPIF, MPEG1, HIF1A, PSME2, TNFSF13B, TET2, ABI3, TLR10,
MYD88, NPC2, GLIPR1, VAMP8, RILPL2, VPS35, TPP1, MIR181A1HG, RIPK2, PARVG, AHR, ARL4C, RIN3, ZFYVE16,
VPS29, HBEGF, CD300A, PTP4A2, ANXA1, C1orf54, PSMA4, AVPI1, GNB4, ETV3, CHMP1B, SRGN, CTSS, PTPN6
CD.cDC2.CLEC10A.FCGR2B
CLEC10A, FCER1A, CX3CR1, CD1E, CLEC4F, CSF1R, MRC1, PLD4, NCF2, RGS18, CD1D, TNFAIP8L2, MNDA, GPAT3,
CD33, PID1, RP11-863P13.3, CFP, IGSF6, CD1C, ZNF385A, LRRC25, HCK, CPVL, CD207, SIGLEC7, HCAR2, KCNMB1,
CSF3R, RP11-404O13.5, CLEC4A, CACNA2D3, P2RY14, ITGAX, SCIMP, HLA-DQB2, AOAH, CSTA, RTN1, CIITA, CLEC7A,
RNF130, INHBA-AS1, PAK1, IL1B, CPPED1, IFI30, GUCY1A3, PILRA, CCDC88A, CD86, CCR2, CSF2RA, RNASE6, MS4A6A,
SLC8A1, HLA-DOA, JAML, FGL2, LST1, MPEG1, CARD9, NDRG2, NAGA, VSIG4, SPI1, RP6-91H8.3, FAM26F, ST3GAL6,
ADAP2, PTGS2, P2RY6, CD4, P2RY13, ARHGAP22, TGFBI, CXCL8, RP11-196G11.2, PKIB, CECR1, FCGR2A, SAMHD1,
SIGLEC10, RASSF4, TCOF1, RAB32, FES, AIF1, LILRB3, FGD2, LGALS2, CD300A, SULF2, TBXAS1, CEACAM4, CCL3L3,
SGK1, SPECC1, C1orf162, FGR, SPATS2L, SIRPA, FAM105A, FCGR2B, PTPRE, SLAMF8, SHTN1, RP11-1143G9.4,
ADGRE2, PLEKHO1, CD68, EGR2, PHACTR1, LGALS9, CA2, MYD88, CD36, CTSZ, MBOAT7, OGFRL1, THEMIS2
CD.EC.FABP1.ADIRF
FABP1, PHGR1, ALDOB, PRAP1, RBP2, KRT8, GSTA1, KRT19, ADIRF, LGALS4, C19orf33, ANPEP, MT1G, KRT20, FABP2,
SMIM24, MISP, KRT18, FABP6, SMIM22, CDHR5, REG1B, MT1E, PIGR, LGALS2, TSPAN8, REG1A, PRSS3, IFI27, MT1H,
CLDN4, S100A14, CCL25, CLDN3, S100A6, EPCAM, DMBT1, ELF3, MYO1A, AKR7A3, AMN, SI, C15orf48, SPINK1,
CDC42EP5, CES2, LGALS3, PPP1R16A, AKR1C3, CLDN7, GSTA2, MT-CO3, MT1M, TMEM54, MT-CO1, CDX1, SLC51B,
MT2A, MS4A8, ESPN, TMC5, KHK, MAOA
CD.EC.FABP6.PLCG2
FABP6, ANPEP, FABP1, CDHR5, ALDOB, PRAP1, RBP2, MIR194-2HG, PLCG2, ADIRF, ELF3, PHGR1, KRT20, C19orf33,
CDKN2B, GSTA1, SMIM24, RP11-380P13.1, PCK1, RP11-160E2.6, SEMA3B, MYO1A, SI, KRT8, MUC3A, CLDN4, DGAT1,
REG1B, MLXIPL, NLRP6, AMN, MYH14, ANO9, EPS8L3, ANGPTL4, SLC5A1, KRT19, GSTA2, SULT1A2, MISP, RP3-
395M20.8, MYO15B, PDZD3, DPEP1, MT-ATP8, CYP3A5, REEP6, CES2, CYP4F12, TM6SF2, CLDN7
CD.EC.GNAT3.TRPM5
SH2D6, TRPM5, BMX, RP11-295M3.4, GNAT3, SH2D7, CCDC129, GNG13, CMYA5, B4GALNT4, OGDHL, GABRA4,
IL17RB, HPGDS, PCP4, POU2F3, MATK, ST18, PLCG2, AZGP1, COL27A1, CCSER1, AVIL, PTGS1
CD.EC.GSTA2.CES3
GSTA2, CES3, EDN3, PLA2G10, AKR1B10, KBTBD11-OT1, SULT1E1, NR1H4, CHP2, SI, PRR15, TMEM45B, SDCBP2,
ADH1C, FAM151A, BTNL3, AADAC, HSD17B2, RP3-417L20.4, KRT20, AKR7A3, ANPEP, LINC01133, SLC51A, PTGR1,
PBLD, GSTA1, CPS1, UNC5D, MT1M, PRSS3, A1CF, CDS1, AKR1C3, SLC6A20, SULT1B1, TMPRSS15, MISP, CMBL,
TMEM253, MAOA, ERBB3, APOBEC1, VIL1, SLC25A34, MT1H, CCL15
CD.EC.GSTA2.TMPRSS15
GSTA2, SLC51B, AKR1B10, AADAC, TMPRSS15, SULT1E1, APOBEC1, C17orf78, METTL7B, SLC7A9, FLJ22763,
PLA2G12B, SLC22A18AS, MEP1A, AKR7A3, MTTP, TMEM171, NR1H4, RBP2, TMEM82, CHP2, BTNL8, OTC, HSD17B2,
SLC3A1, AC004754.3, SDCBP2, ADGRG7, XPNPEP2, CMBL, CYP2C18, ACKR4, SLC10A2, CDKN2B, TMEM45B,
TMEM253, COL17A1, MALL, FAM151A, KCNJ13, RP11-680F8.1, BTNL3, TINAG, TMEM30B, CPS1, CDS1, MS4A8,
ARHGDIG, CKMT1B, PRR15L, TM4SF20, TSPAN1, ABCG2, ESPN, ACE2, CLRN3, UGT2A3, TJP3, ADH1C, GPD1, RP3-
417L20.4, A1CF, CKMT1A, TM6SF2, HSD11B2, EDN2, B3GALT5, LGR4, MRAP2, DDC, MYO1A, GSTA1, MAOA, MYL4,
PTGR1, ACOT11, PLCB3, RP11-542M13.2, SLC51A, GNA11, CCL15, PDZD3, PAPSS2, ADTRP, TMEM92, STAP2,
SULT1A1, C10orf99, BCL2L15, PDE4C, SLC39A5, KHK, PPP1R14D, SULT1B1, NAALADL2, CDKN2B-AS1, ENPEP, SI,
KBTBD11, SERPINB5, DHRS11, LAD1, PVRL3, AKR1C2, FAM3D, TTC36, TMPRSS2, VIL1, ARSE, GIPC2, TMPRSS4, CDH17,
HNF4G, MYH14, GGT6, SLC37A4, NAT2, MGAT4B, C2orf72, AP1M2, KRT20, MOGAT3, SEMA6D, HOXB7, SLC5A1,
TMEM37, IGFBP2, PBLD, TMEM54, AKR1C3, DEGS2, TRIM15, GUCY2C, PHYHIPL, MOGAT2, MISP, GATA6, SMIM24,
ABHD11-AS1, USH1C, HOOK1, PKP3, RAB25, FXYD3, CXADR, NR5A2, RP11-532F12.5, S100A14, ANPEP, CCDC64B,
PRSS3, SEMA4G, TKFC, ARRDC4, CREB3L3, DNMBP, MAL2, PRR15, SLC44A3, DENND2A, UGT2B17, PTPRH, MGST1,
PPP1R16A, CDHR5, SERINC2, RNF128, SLC34A2, BAIAP2L2, PKP2, PLPP2, C1orf210, AOC1, PCK2, SLC22A18, SCIN,
FAAH, GPA33, CDX1, AMN, XK, F2RL1, AGR3, CORO2A, CFI, FAHD1, PCK1, MARVELD3, TMC4, FABP6, DSC2, PPIC,
MUC3A, SLC44A4, PTK6, PLS1, GLYCTK, LINC01207, HPGD, KIAA1522, KLF5, EDN3, NHSL1, S100A16, OVOL1, SLC13A2,
SELENBP1, ACSS2, FABP1, PRSS8, MYO1D, CDX2, GJB1, TOM1L1, FAM84A, KRTCAP3, SHD, SPINT1, XDH, SOWAHA,
RETSAT, CES2, TMEM139, FAM3B, SLC26A3, ATP8B1, GCNT3, FABP2, CHMP4C, ALDOB, SIAE, C1orf106, SEMA3B,
ESRP1, AKAP1, CLDN15, CAMK2N1, KCTD3, ERBB3, SH3BGRL2, CFTR, CCDC58, LCN2, MUC13, CDC42EP5, IFT172
CD.EC.NUPR1.LCN2
UGT2B17, MGST1, PDZK1IP1, REG1B, ALDOB, GPX2, LCN2, DMBT1, AGR3, RAMP1, AKR1C3, TSPAN8, EPCAM, KLF5,
PLA2G2A, SLC12A2, FAM3B, OLFM4, SPINK1, RGMB, LGR5, RNF43, RAB25, CLDN3, PLPP2, DPEP1, CLDN7, ADH1C,
EPHB2, PIGR, LGALS4, EDN1, PPP1R1B, PKP2, CFTR, PRSS3, AGR2, RNF186, CEACAM5, ELF3, KIAA1324, KRTCAP3,
KRT19, CXADR, PPP1R14D, SMIM22, CDX1, MT1G, KRT18, MAOA, MT1E, DSG2, ALDH1A1, DDAH1, CD9, SMOC2,
C2orf82, DSP, GPR160, TMC5, KRT8, CDCA7, FERMT1, GATA6, CDX2, CLDN4, URAD, NHSL1, GUCY2C, CCL25, STAP2,
MGST2, STXBP6, SELENBP1, ETS2, DUOX2, KIF12, GJB1, CNN3, TMEM54, CERS6, PHGR1, CPS1, IFI27, CLDN15,
LGALS2, RARRES1, VIL1, AKAP1, GOLM1, ATP1B1, CLDN2, REG3A, KCNJ13, AZGP1, CXCL3, ATP10B, GIPC2, EHF,
REG1A, CMBL, OTC, MAL2, C15orf48, SNHG18, MYO6, GGT6, ATP2C2, FABP2, CYB5A, LINC01207, MT1H, CLRN3,
UGT2A3, MDK, IFT172, GP2, PVRL3, S100A14, AQP1, ERBB3, RP11-532F12.5, PBLD, GPRC5A, BCL2L15, PRAP1, ITGA6,
NR5A2, SLC44A3, SLC39A5, TMEM45B, DUOXA2, PSMG1, MET, MUC1, LIMA1, SOX9, DDC, OAT, ASCL2, S100A16, SI,
CISD3, GDF15, NR1H4, SUCLG1, GALNT3, TRIM2, SLPI, SH3YL1, CDH1, ATP8B1, FAM84A, KCTD3, DEGS2, SORD,
EPHB3, CCL28, MRPS35, TCEA3, AP1M2, MT1F, LRIG3, CAMK2N1, FXYD3, ABO, PTGR1, CCND1, SNX7, SLC39A14,
PKP3, CBLC, IGFBP2, MLEC, MYB, PRSS8, ESRP1, HOXA9, COA3, PPP1R16A, NOS2, AKR7A3, ASS1, QTRT1, SFN,
C19orf33, USH1C, TMPRSS2, NUPR1
CD.EC.OLFM4.MT-ND2
REG3A, OLFM4, KRT18, GPX2, LCN2, SPINK1, PLA2G2A, PIGR, PRSS2, REG1A, TSPAN8, REG1B, KRT8, RAMP1, ELF3,
DEFA6, PHGR1, CLDN3, CLDN4, DMBT1, RNF43, MT-CO3, MT-CO2, MT-CO1, MT-CYB, MT-ND4, MT-ND2, MT-ND1,
SMIM22, MT-ATP6, EPCAM, LGR5, AGR2, GDF15, SLC12A2, MT-ND3, MT-ND5, TFF3, DEFA5, LGALS4, MT1G, EPHB3,
EDN1
CD.EC.SLC28A2.GSTA2
GSTA2, SLC7A9, MS4A8, SLC28A2, RP3-417L20.4, SLC6A20, AC005550.3, COL17A1, RP11-680F8.1, TMIGD1, ABCG2,
FAM151A, ARHGDIG, SDR16C5, AC021218.2, SLC10A2, LYPD8, SLC3A1, SLC51A, SDCBP2, C17orf78, MME, AKR1B10,
SERPINB5, ABCG5, AADAC, FLJ22763, SLC5A1, C6orf132, SLC51B, CDKN2B, TMEM45B, GJB3, SI, HSD17B2, TINAG,
GDA, TMPRSS2, OTC, ACE2, KBTBD11-OT1, PPP1R14D, AKR7A3, MARVELD3, ANXA13, PBLD, MGAM, SULT1B1,
ARL14, WIPF3, GSTA1, MYH14, PLA2G12B, CPS1, PTPRF, TM4SF20, ENPEP, UGT2A3, EDN2, CTSE, CYP2C18, BTNL8,
SLC39A5, REG3G, MEP1A, EPB41L4B, MAOA, C1orf106, SLC26A3, ADH1C, MTTP, SMIM24, CDH1, MOGAT3, AOC1,
DHRS11, SULT1E1, CDS1, TMEM92, PTGR1, KRT20, CHP2, CCL15, PLCB3, MALL, FXYD3, TST, GNA11, DEFB1, NR1H4,
CDHR5, PRSS3, SLC44A4, EDN1, USH1C, TMC4, STAP2, APOBEC1, CMBL, MISP, HGD, LAMB3, AKR1C3, FAM101A,
MYO1A, CKMT1A, SEMA6D, FMO5, LAMA3, CLDN15, MYO5B, TLCD2, ANPEP, PAQR5, FAM3D, XDH, CBLC, DSC2,
UGT2B7, FAM3B, TMEM54, LINC01133, SERINC2, TP53I3, TM6SF2, KLF5, MGST1, ESPN, TMPRSS4, VIL1, S100A14,
CGN, KIAA1522, ERBB3, PPARGC1A, ITGB8, TSPAN1, MUC13, METTL7B, TMEM253, TMEM37, DSP, CYBRD1, FABP1,
CFTR, PTPRH, CYP27A1, CDH17, MTMR11, A1CF, IRF6, BTNL3, ANO10, HEPH, RAB25, HNF4G, GUCY2C, TMEM164,
HKDC1, RETSAT, LAD1, ARHGEF5, C19orf33, CES2, HNF4A, RAPGEFL1, PLS1, FABP2, LRRC66, MYRFL, CLRN3, AGR3,
ALDOB, AP1M2, MOGAT2, SLC37A4, HEBP1, TMEM236, IYD, ANKRD9, LMO7, NELL2, LGR4, CHMP4C, C1orf115,
SPINT1, ARHGEF35, GOLT1A, FBP1, SHROOM3
CD.Endth/Ven.LAMP3.LIPG
VWF, PLVAP, ENG, MGP, COL4A1, SELE, HSPG2, ACKR1, COL4A2, RAMP3, COL15A1, VWA1, PCAT19, CRIP2, RAMP2,
MADCAM1, PRSS23, CLDN5, PRKCDBP, EGFL7, HYAL2, NPDC1, CD93, TGM2, SLCO2A1, PTRF, FAM167B, SELP,
PCDH17, CALCRL, ECSCR-1, SERPINE1, CLEC14A, APLNR, IL3RA, ARHGAP29, ITGA5, CTHRC1, MCAM, RAI14, FLT1,
EMP1, FAM198B, SOX18, LAMP3, RCAN1, ESAM, P2RY6, ADGRL4, FSTL1, CDH5, MMRN2, HTRA1, RASIP1, ZNF385D,
LIPG, IL33, PXDN, ROBO4, MEOX1, EMCN, SEMA3F, CYYR1, CD34, DPYSL3, ERG, RHOJ, JAM2, KDR, RAB3C, TIE1,
TMEM255B, CXorf36, MAP1B, PLA1A, ADAMTS9, OLFM1, S100A3, SHANK3, FAM110D, MAPK11, NPR1, SPNS2,
RAPGEF4, DOC2B, GPR4, ACVRL1, MYCT1, TINAGL1, GALNT18, MMRN1, JAG2, FNDC4, LAMA5, SNAP25, NEURL1B,
ARHGEF15, SYNPO, TM4SF1, CSF3, CXCL11, CCDC3, KANK3, TLL1, NDST1, CRIM1, TSPAN7, NR2F2, PTPRB, FGD5,
SERPINH1, TEAD4, ADAMTS4, EPAS1, FOXC1, NOTCH4, PALMD, FCN3, FZD4, DYSF, COL18A1, SPARCL1, BCAM,
ADAM15, LDB2, RP11-536O18.1, SAMD14, HECW2, NRP1, NNMT, RNASE1, PLCB1, MPZL2, TEK, NOVA2, PLXND1,
PDGFB, PODXL, ARHGAP23, HEG1, RBP5, ADGRF5, ADGRL2, GNG11, ADCY4, IGFBP4, SPARC, LEPR, PECAM1, WWTR1,
A2M, SEMA6B, LAMC1, MATN2, NFIB, DCHS1, LHX6, CFI, BCL6B, TNFAIP1, FAM129B, HSPA12B, EHD2, BMPR2,
CCM2L, NOS3, PPM1F, EPHB4, FSCN1, SNCG, CPLX1, SPRY4, ADAMTS1, CCL23, LIMS2, PKP4, MCTP1, C1R, SHROOM4,
LAMB2, CPE, CNTNAP3B, CAV2, CSF2RB, OAZ3, LTBP1, CSRP2, CFH, FAP, COX7A1, CAV1, NUAK1, APOL1, HSPB8,
CD109, C1orf54, TRIM47, RASA4, IGFBP7, SLC35G2, SVIL, TNS2, THSD7A, EVA1C, DOK5, PTPN14, A4GALT, VEGFC,
SEPN1, MEOX2, CLEC1A, SCARF1, ANGPTL2, DOCK6, NRN1, PLS3, CALD1, SMAD6, MALL, GRB10, HHEX, THBS1,
CD200, EXOC3L2, ZNF521, IPO11, BCR, ANKRD65, TGFBR2, RAB13, CNKSR3, DLC1, HOXD8, LTF, BMP1, ADGRG6,
NOSTRIN, HAPLN3, PDLIM4, SNTB2, PREX2, SNCA, ADGRG1, MPDZ, GJA1, RBPMS, SHE, PVRL2, SPRY4-IT1, FLT4,
S100A13, TM4SF18, APP, ECE1, NRGN, STXBP1, CDKN3, SLC9A3R2, MAPK12, GRASP, RBMS2, COL6A2, AQP1, STC2,
HYAL1, DOCK9, MKL2, WTIP, LARP6, ABL2, PCDH1, SLC29A1, CAMSAP2, LMO2, FAM101B, FAM65A, SMAD9, ELK3,
ID1, CNN3, PTPRM, CLIC4, FLNB, KIAA1462, FKBP10, FAM107A, AFAP1L1, TUBB6, SPTBN1, GOLM1, NCKAP1, SHC1,
TCF4, ARHGEF12, THY1, CASKIN2, SERPING1, EFNA1, BOK, ADAMTS7, C4orf32, BACE2, GPRC5B, PDLIM3, ITGAV,
SPHK1, UBTD1, YPEL2, S100A16, SORBS3, PPIC, LOXL2, DBN1, PKN3, SMAD1, FAM171B, TMEM173, PXN, LMCD1,
ENPP2, KLF7, FAM43A, RRAS, NT5E, TSPAN9, TBCD, FKBP9, CMIP, CD151, LDLRAD3, INSR, ACTN1, ROBO1, PON2,
S1PR1, STOM, PDLIM5, TMEM204, PCGF2, ARAP3, VAT1, HAGLR, FERMT2, IFI27, AGAP2-AS1, STAB1, WARS, CPD,
CTTN, PAM, DUSP23, AMOTL1, IFITM3, EFNB1, TNFRSF1A, WBP5, KAZN, RGS3, TJP1, TCN2, DPYSL2, PHLDB2, FBN1,
HOXD9, SOX17, C8orf4, F2RL3, PLOD1, ITGB4, MAST4, ENTPD1, GNB4, PDLIM1, CYP2C9, PRCP, RASSF9, LINC01235,
TMTC1, MMP14, RND1, LHFP, RDX, CYP1B1, UPP1, CYR61, SOCS2, TPD52L1, CSGALNACT1, PTPRG, ARHGEF10,
TMEM2
CD.EpithStem.LINC00176.RPS4Y1
RNF186, LGR5, KIF12, KIAA1324, RGMB, CDC42EP5, PPP1R1B, AQP1, CCL25, PLPP2, RAB25, CDX1, SLC44A3, RAMP1,
PDZK1IP1, IFT172, USH1C, EDN1, AKR1C3, STAP2, HMGCS2, LINC00176, RNF43, MGST1, STARD10, SELENBP1,
ALDH1B1, RP11-357H14.17, VIL1, S100A14, PRSS3, C9orf152, GPX2, MISP, ASCL2, CDCA7, MTMR11, ATP2C2, FAM3B,
SLC39A5, KLF5, TMPRSS2, IGFBP2, CLDN15, TMEM171, PKP2, CLDN4, CLDN3, ERBB2, ADH1C, HNF4A, CLDN7,
SLC12A2, FXYD3, ERBB3, CMBL, TSPAN8, CDX2, KRTCAP3, FABP2, CFTR, ALDOB, EPHB2, PTPRF, TMEM98, HOXB9,
PBLD, EPCAM, SPINT1, SOX9, SLC44A4, C19orf33, NHSL1, IRF6, SEZ6L2, SMIM22, CDH17, MYO6, RP11-519G16.5,
LGALS4, STX19, CBLC, GATA6, LRIG3, EPHB3, SFN, FERMT1, OLFM4, CDH1, WNK2, C1orf106, GPRC5A, PPP1R16A,
ELF3, ST14, ESRP1, DDC, SDC4, STXBP6, TMC4, TRIM2, ROBO3, CYP3A5, KRT19, AP1M2, AMN, AGR3, TST, MAL2,
TMEM54, FAM84A, PLEKHS1, OTC, SMIM24, GDF15, MT1M, GABRP, DSG2, PKDCC, OVOL1, BCL2L14, TMEM45B,
ENPEP, VDR, CHMP4C, GIPC2, PROM1, CHP2, PIGR, CAMK2N1, GGT6, KRT8, QTRT1, SPINK1, PPP1R14D, MYO10,
S100A16, LINC01207, KRT18, NOXO1, HNF4G, PERP, DSP, ATP8B1, C2orf82, SOX4, MAOA, AGR2, SERPINB5, LLGL2,
GSTO2, PTGR1, TCEA3, TSPAN6, DDR1, ZNF703, CERS6, MLXIP, DDAH1, SNHG18, SMOC2, CCL15, PRAP1, MT1G,
GUCY2C, AKAP1, EPHX2, GPR160, CTBP2, ALDH1A1, DSC2, NFIB, CASP6, CENPV, IMPA2, PCK1, CDK6, MPST, MT1E,
DHRS11, CXADR, ETS2, DEGS2, CCDC64B, GSTA1, CDC42EP4, CD9, HDHD3, CCND1, MUC3A, UGT2A3, SNHG19,
FAM3D, ATP10B, ABO, SORD, KCNE3, NR5A2, PRSS8, ARSD, TINAG, HOXA10, TRABD2A, TSC22D1, CTTN, LAMB3,
ABHD17C, PHGR1, PDGFA, EFNA1, MUC13, RHOB, TSPAN12, PLCB3, TM4SF5, PRR15L, RNASE1, MGST2, PBX1, OSR2,
GCNT1, CPS1, CYB5A, CHDH, NFIA, UGT2B17, TJP3, CLRN3, POF1B, SEMA6A, MARVELD3, ARHGEF10L, AKR7A3,
ITGA6, ST6GALNAC1, CNN3, FAAH, LSR, ATP1B1, LGR4, ARL14, MYH14, CYP4F12, HOXA9, MT1F, CCDC58, KIAA1522,
LRRC75A, GRTP1, MDK, JUP, ALDH2, NTHL1, CCDC34, KCNQ1, FUT3, PCBD1, APP, PGAP1, TMC5, QPRT, NOB1, HEPH,
SAMD5, AC013461.1, ACSS2
CD.Fibro.CCL19.IRF7
FBLN1, LINC01082, CCL11, CXCL6, CYGB, NNMT, EFEMP1, DCN, MFAP4, SOD3, C3, PLAC9, TDO2, CXCL1, EMILIN1,
PTN, COL3A1, CCL2, CTSK, LUM, CFH, PCOLCE, EDIL3, ADH1B, C1S, COL6A2, COL1A1, MMP2, PTGDS, KCNS3, RBP5,
TCF21, ECM1, C1R, RARRES2, EFEMP2, MXRA8, MEG3, CDH11, IL34, GGT5, COL6A3, C7, TGFB1I1, COL1A2, CXCL12,
LOXL1, HAPLN1, CCL19, CALD1, PDPN, FABP4, HOXC9, FBLN5, SERPINF1, TFPI, CCL13, GPX3, CP, ABCA8, TNXB,
IGFBP6, BMP5, FHL1, CXCL14, MYL9, TPM2, NKX2-3, DDR2, ANGPTL1, PCDH18, PLPP3, FILIP1L, SFTA1P, FIBIN, RBP1,
PMP22, VCAM1, ADAMDEC1, QSOX1, SGCE, NID1, PLPP1, SPARC, F10, PTGES, SNAI2, C16orf89, LAMA4, FNDC1,
IGFBP4, HOXC8, PHLDA3, FGF7, PLTP, PROCR, TWIST2, FENDRR, OLFML1, SERPING1, BMP4, SERPINE2, THY1, IGFBP7,
PDLIM3, OLFML3, S100A13, CFD, TPST1, MFGE8, PROS1, CRISPLD2, EVA1A, COL6A1, FHL2, STMN2, GPX8, SPARCL1,
GLT8D2, ITIH5, PLAU, NDN, LTBP4, TMEM119, FAM92A1, SERPINH1, PRKCDBP, PTGIR, ELANE, CCDC80, A2M,
PDGFRA, DKK3, TAGLN, TMEM176A, OSMR, FAM216B, TM4SF1, RRAS, TMEM47, CPXM1, LPAR1, RNF112, TPPP3,
SNCAIP, FXYD1, VCAN, PLAT, FXYD6, EFCC1, ANTXR1, HTRA3, COL18A1
CD.Fibro.IFI6.IFI44L
TDO2, CYGB, DCN, PTN, MFAP4, LUM, FBLN1, SOD3, C3, EMILIN1, CTSK, MMP2, PROCR, C1R, CCL2, GGT5, COL3A1,
HOXC8, TCF21, CXCL14, RARRES2, ABCA8, RBP5, IL34, COL6A1, CFH, FOXF1, C1S, COL6A2, TM4SF1, COL1A1, HTRA3,
CXCL6, PLAC9, THY1, GPX3, NNMT, FABP4, HAPLN1, FGF7, EFEMP1, LINC01082, EFEMP2, MXRA8, TPM2, ADAMDEC1,
IGFBP6, SERPINF1, RBP1, PCOLCE, CXCL1, CCL19, CHI3L1, COL1A2, NKX2-3, LOXL1, LAMA4, EDIL3, CALD1, FHL1,
PCDH18, PLAT, EVA1A, HAS2, CRISPLD2, BMP5, PLPP1, FXYD6, CXCL12, IGFBP7, SFTA1P, PTGES, GPX8, MYL9,
PDGFRB, BGN, CP, PDGFRA, ECM1, TFPI, SPARC, NDN, STMN2, QSOX1, KITLG, APOE, PTGDS, RBPMS, PLPP3, NR2F1-
AS1, BICC1, SERPINH1, NUPR1, VASN, ADH1B, FHL2, ALDH1A3, CCL8, CFD, IDO1, COL6A3, CCL11, TSPAN4, MXRA5,
TMEM119, SLITRK6, TAC3, PMP22, SERPING1, A2M, FKBP10, S100A13, BMP4, EPHX1, MAB21L2, VCAM1, PPP1R14A,
DKK3, NBL1, MEG3, SNAI2, ACHE, DDR2, PRKCDBP, TUSC3, MFGE8, DIO2, IFI27, ADGRL3, FBLN5, TGFB1I1, NID1,
SCGB1B2P
CD.Fibro.TFPI2.CCL13
CFD, CCL13, PTGDS, CCL11, LUM, DCN, RARRES2, APOE, A2M, ADAMDEC1, FBLN1, APOD, PLPP1, MFAP4, CCL2,
IGFBP5, C1S, CXCL14, CALD1, SFRP1, TFPI2, PLTP, IGFBP7, COL1A2, GNG11, ADH1B, CCL8, RBP1, SPARCL1, C1R,
LGALS1, GPX3, VCAN, COL3A1, CXCL1, CFH, TMEM176B, NGFRAP1, S100A13, TCF21, PPP1R14A, SERPINF1, IGFBP6,
PLPP3, PDLIM1, CXCL6, TFPI, COL18A1, FHL2, MMP2, IGF1, IFITM3, TIMP1, GSN, PMP22, HAPLN1, RAB13, EDIL3,
COL6A2, SPARC, QSOX1, TMEM176A, GGT5, SFTA1P, TGFBI, LTBP4, LGALS3BP, TIMP3, IGFBP4, ECM1, CTSK, ABCA8,
SPON2, NUPR1, PLAT, CLEC11A, FHL1, SGCE, SERPINE2, EMILIN1, FXYD1, CYGB, PLAC9, TPM2, RHOBTB3, CLEC14A,
PDGFRA, FIBIN, DDR2, FBLN5, COL14A1, EDNRB, NDN, GSTM3, PRKCDBP, LINC01082, OLFML3, TMEM98, CXCL12,
RBP5, C1orf21, NID1, DPT, COL6A3, MYL9, BMP4, RBPMS, EFEMP2, SCARA5, DKK3, COL15A1, NNMT, COL6A1,
EFEMP1, C7, SNAI2, MEG3, PAPPA, LAMA4, STMN2, PCDH18, ANGPTL1, HOXC9, LTBP1, LINC01116, AKAP12,
TNFAIP6, IL34, GLT8D2, MXRA8, NEGR1, TSPAN2, HGF, PTGES, ANGPTL2, TMEM150C, ELANE, COLEC10, FSTL1,
KCNS3, GRK5, CLMP, PCDH7, ABCA6, GJA1, SAMD11, NOVA1, SELM, CDH11, ANGPTL4, EPHX1, ADGRL3, SMPDL3A,
CRISPLD2, CST3, COL5A2, EMP2, WBP5, NKX2-3, BMP5, MTRNR2L8, C11orf96, SLIT2, GSTM5, CD63, PTN, HOXC8,
FBN1, TM4SF1, CRYBG3, EMID1, TCEAL7, COL1A1, PLEKHH2, VIM, PCOLCE, RAB34, SERPING1, SRPX, NFIA, PROCR,
IL6ST, TGFB1I1, IFITM1, CP, CCDC80, SULF1, FILIP1L, LAPTM4A, PTMS, RP11-14N7.2, NDUFA4L2, EPHA7, CTSC,
CNRIP1, EHD2, LAMC1, FKBP10, MXRA5, PROS1, FN1, MAMDC2, GPC6, RPS6KA2, IGFBP3, CHL1, COL5A1, EID1,
COX7A1, STEAP1, ACVRL1, PLXDC2, COL4A2, PTCH1, NBL1, APOC1, FAM127A, FAM150B, COLEC11, PGRMC1, TUSC3,
OLFML1, TNXB, CPQ, ADGRL2, FGF10, AEBP1, CPNE8, LPAR1, TSPAN4, HPSE2, SDC2, AKR1B1, TNFRSF11B, LAMB1,
FGF7, SCT, RUNX1T1, PGF, TRIL, ADAM28, TNFRSF1A, ISLR, ENPP6, HTRA3, PLEKHA5, MRGPRF, TNFSF10, ALDH1A3,
CYBRD1, SGCD, HSD11B1, CTSL, MATN2, MAB21L2, CD302, MAPK10, MFGE8, TPM1, PBX1, TCEAL4, C16orf89, TRPA1,
LOXL1, PAM, WNT2B, ASPN, TPST1, TMEM100, IDO1, BST2, CNN3, EFCC1, LTBP2, TPPP3, CH25H, ZNF667-AS1,
HOXC6, SRPX2, ATRAID, HOXB-AS3, IL1R1, KLHL23, PXDN, TWIST2, FGFR2, ANTXR1, DSTN, ARHGAP20, SOD3, LHFP,
PDGFRB, RBMS3, PRKG1, GPX8, MRFAP1, CRIP2, SERPINH1, BDH2, TSC22D1, GSTA4, UBE2E3, SGIP1, MRC2, NFIC,
F10, ANXA2, LEPROT, FOXF2, IFI27, KANK2, S100A6, WFDC1, RAI2, ZEB2, ZCCHC24, HSBP1, PRR16, GNAI1, LRP1, ST5,
FKBP7, FENDRR, WWTR1, VCAM1, TIMP2, CXXC4, RP11-356J5.12, NR2F1-AS1, APCDD1, CTSF, HNMT, RHOC, SEPW1,
MTRNR2L12, P4HA2, PITX1, TAC3, PPIC, LOX, ACTA2, KAZN, NSG1, TMEM59, ZNF503, KITLG, TBX2-AS1, MIR497HG,
PLAU, ALDH2, SLC16A4, DNM3OS, NCAM1, HAS2, ANXA5, CDKN2C, TCEAL8, FGFR1, TMEM47, TBX2, DSEL, TNS2,
RCN1, MMP23B, PTGFR, VKORC1, MGP, RP11-834C11.4, ENC1, FBLIM1, DMKN, G0S2, TPBG, WLS, HTRA1, EFHC1,
MUC12, FARP1, TUBA1A, GAS7, ADAMTS5, SPON1, FAM92A1, TCF4, RHOJ, NENF, ASPA, SLC9A3R2, SMIM10, LXN,
NDUFA4, FTH1, PHPT1, IFIT1, LAMB2, EXTL2, BSG, SCARB2, CRTAP, ITGB1, SCD5, RRAGA, FAM114A1, ESD, RPL7,
TDO2, CBR1, SYNPO2, PDK4, PKIG, BICC1, TMEM45A, CCPG1, PDLIM4, PDPN, ADH5, NR2F2, FAM13C, CD151,
ANKRD35, PTPRK, H1F0, ITIH5, LURAP1L, ITGB5, FERMT2, TPT1, MAP1LC3A, FABP5, 11-Sep, ARL4D, FAM198B, RRAS,
TXNIP, HEBP1, FGFR4, HOXA7, EEF1D, PARVA, ITM2B, ANTXR2, THY1, PHYHD1, MEIS2, DPYSL3, EEF1A1, CTDSPL,
COL12A1, SKP1, FAM229B, DYNLL1, RDX, LGALS3, SEPT4, TAX1BP3, TMEM205, FUCA2, RPS4Y1, COL4A1, CEBPD,
NEXN, CD81, SMIM3, PPIB, LMCD1
CD.Goblet.RETNLB.ITLN1
CLCA1, ITLN1, RETNLB, FCGBP, FXYD3, REP15, HEPACAM2, SPDEF, LRRC26, ATOH1, GALNT8, LINC00261, SPINK4,
KLK12, ST6GALNAC1, REG4, S100P, FAM3D, REG3A, RP11-234B24.2, CEACAM5, SERPINA1, HES6, STARD10, TFF3,
GPX2, SPINK1, SMIM22, CREB3L1, BCAS1, LGALS4, MUC1, ENTPD8, LCN2, PLA2G2A, PRUNE2, L1TD1, AGR2, KRT18,
ANO7, TCEA3, WFDC2, TMEM61, ZG16B, KIAA1324, PIGR, ERN2, FOXA3, EPCAM, SCNN1A, LINC01207, SLC12A2,
RAB26, REG1B, RAMP1, AGR3, FAM3B, EPHB3, AZGP1, IGFBP2, FABP2, MLPH, KRT8, CAMK2N1, KRT19, COL16A1,
KLK1, ELF3, CLDN3, CLDN4, S100A14, MDK, GUCA2A, ARFGEF3, CLDN7, DMBT1, MB, IFI27, REG1A, NOXO1, ASRGL1,
TSPAN1, GDF15, CDC42EP5, MUC4, PCSK1, RAB25, BACE2, TPM1, PROM1, RAP1GAP, TSPAN8, RBBP8NL, CRACR2A,
TPSG1, GMDS, CTD-2015H6.3, RNASE1, FRZB, MUC13, KLF5, NPDC1, NEURL1, CDH1, C9orf152, PHGR1, MAL2,
ATP2C2, MGST1, 1-Mar, PPP1R1B, URAD, RASD1, DNAJC12, BAIAP2L2, FUT3, ANXA13, DSG2, LYPD8, PROX1, SOX9,
NQO1, DSP, PLEKHB1, ITGA6
CD.Goblet.TFF1.TPSG1
REG4, CLCA1, FCGBP, TFF1, ITLN1, FXYD3, CEACAM5, S100A14, ZG16, S100P, ST6GALNAC1, KRT20, FAM3D, TSPAN1,
REP15, PPIC, NPDC1, FAM3B, TPSG1, LINC00261, LINC01207, BCAS1, ANG, FHL2, CREB3L1, SPDEF, MUC1,
HEPACAM2, MUC13, MLPH, FAM177B, URAD, PLA2G10, MAL2, CDH1, LRRC26, ZG16B, FOXA3, RP11-234B24.2,
ARFGEF3, GALNT5, SDCBP2, B3GNT6, CEACAM6, MBOAT2, ENTPD8, SCNN1A, MUC4, DNAJC12, CAPN8, GALNT12,
TTC39A, ABCA4, RAP1GAP, FFAR4, GALNT8, MUC5B, STXBP5-AS1, CGREF1, RBP4, TMEM61, ERN2, C9orf152, KCNK1,
LIPH, GALNT7, HES6, GALE, AGR3, B3GNT7, PRUNE2, SGMS2, NQO1, ATOH1, BACE2, TCEA3, GMDS, FABP2, SPINK4,
GNE, GALNT3, TMEM184A, CANT1, ANXA13, PLEK2, SERPINA1, STARD10, PTPRF, GFPT1, KDELR3, KIAA1324,
SERINC2, SMIM22, SLC44A4, RASEF, CA12, S100A16, MB, ATP8B1, KAZALD1, NEURL1, CLDN7, ASPH, RARRES1,
ESRP1, PIGZ, F3, PPP1R1B, TM4SF5, RAB25, CLDN4, AGR2, ERRFI1, GNPNAT1, CREB3L4, ASS1, GPR153, LGALS4,
CKAP4, CAMK2N1, CLDN3, RP11-462G2.1, TNFRSF21, SEC16A, TMCO3, SPINK1, COL16A1, RETNLB, EPCAM, PHLDA2,
TMEM125, TMPRSS4, PYCR1, SYT13, VIL1, CRACR2A, IFT172, C8orf4, TRPA1, TOM1L1, STXBP6, ASRGL1, C3orf52,
BMP2K, SH3BGRL2, FMOD, CTD-2015H6.3, SHROOM3, CCL15, LMO7, PARD3, FUT3, AKR7A3, KLK1, SLC12A8, BTNL3,
TFF3, RAB15, ITGA2, TSPAN13, TSTA3, TSPAN8, HK2, UNC13B, PKP3, AOC1, TSPAN15, SLC35A3, CMBL, CRB3, KLF5,
AGPAT2, KLF4, B3GNT3, RNASE1, GDF15, TMEM141, DSG2, DACH1, RAB3D, RAB27B, FAM114A1, CLRN3, GPX2,
ALDH1A1, KCNK6, PIGR, NUDT8, TPM1, EDEM3, CDX2, FRZB, KRT19, SLC12A2, CYB561, KIF9, CCND1, EPB41L4B,
CHMP4C, DDAH1, ELF3, ABHD17C, CDH17, PARM1, HSD11B2, ST14, A1CF, SLC7A11, TMEM54, MIR22HG, CAPN5,
SEC24D, PERP, SLC39A5, SNHG18, MYO1D, DNAJC10, EHF, PRR15L, COMTD1, GPA33, ARL14, CDC42EP5, UGT2B17,
ANXA4, CMIP, NEDD4L, RAB27A, RAMP1, EFNB2, DSP, TMED3, RBM47, CDX1, SLC39A11, QSOX1, IFI27, HMGCR,
GCAT, STAP2, ACSS2, ARHGEF38, LYPD8, DSC2, PKP2, TM9SF3, ALDOB, MT1G, EMP2, MT1E, ATP2C2, MDK, SYTL2,
PHGR1, HEPH, C6orf132, KRT8, IRF6, CASP7, CTTN, APP, MGLL, AMN, MEP1A, IMPA2, F11R, ETS2, SLC50A1, COG5,
KRT18, SPINT2, HMGCS1, PLS1, ARPC1A, SLC39A7, ATP1B1, EIF4EBP1, TMEM30B, MCU, GSN, TP53INP2, SQLE,
MGST1, IL13RA1, C11orf24, PGM3, PDXDC1, ARHGAP32, C19orf33, LSR, SPATS2L, SCD, PDZK1IP1, TMC5, MISP, FRYL,
LAMA3, ADIPOR2, PLCB4, PCBD1, RHOB, ZNF703, KRTCAP3, USH1C, AK1, C1orf106, NDRG1, HDLBP, DHCR24, MT1X,
LDLR, BCKDHB, CD9, PRIMPOL, LGALS3, IER3, RP11-532F12.5, ADM, ICA1, HBEGF, PLPP2, SLC39A14, KCNE3, ARFIP2,
CYP3A5, WIPI1, GSKIP, B3GNT5, CYSTM1, COG3, AQP3, ALDH2, WASL, RASSF6, MMAB, RP11-357H14.17, GNAQ,
MGST2, TRIQK, SERPINB6, ERGIC1, TSPAN3, SLC17A5, RASSF7, YIF1A, ITGA6, PKDCC, CLCN3, MAGED1, ZNF511,
PRRC1, ATF3, MARVELD3, CTNNBIP1, STARD4, NDFIP2, LGR4, SEC61A1, CXADR, IBTK, FAM120A, PALLD, MFSD6,
ROCK2, BOLA3, RAB13, CRACR2B, RND3, XPNPEP1, PIP5K1B, DST, CAMK2D, CMAS, HPCAL1, DYRK4, ODC1, SOX9,
MYO10, MT1F
CD.Mac.APOE.PTGDS
C1QB, C1QA, APOE, C1QC, SEPP1, APOC1, LYZ, SLC40A1, DNASE1L3, CST3, FTL, CCL18, TYROBP, PSAP, AIF1, MMP12,
CTSC, FUCA1, FCER1G, HLA-DRB1, CTSD, CD68, MS4A6A, IGSF6, CTSB, NPC2, HLA-DPA1, HLA-DRA, CTSS, MMP9,
GPNMB, HLA-DQA1, LIPA, ACP5, GRN, FCGRT, HLA-DPB1, CD14, LGMN, MS4A7, PLD3, ATOX1, CPVL, HLA-DRB5, FGL2,
MS4A4A, PLA2G7, TMEM176B, CTSL, HLA-DMA, PRDX1, HLA-DQB1, ASAH1, CD74, CTSZ, HLA-DMB, SAT1, GPX1,
CD63, RP11-1143G9.4, CAPG, ITM2B, CXCL9, FOLR2, FAM26F, CXCL8, CTSH, GLUL, LY96, LST1, SOD2, CREG1, FTH1,
S100A11, C1orf54, IL18, CCL3, LILRB4, RNASE6, RNASET2, PLA2G2D, CYBB, BRI3, VSIG4, IL1B, TMEM176A, FCGR3A,
ATP6V1F, SPI1, CSTA, VAMP8, ENPP2, PLAUR, C2, MPEG1, RNF130, ITGB2, CSF1R, CYBA, PPT1, JAML, AXL, DAB2,
CLEC10A, TNFSF13B, MRC1, SERPING1, CTSA, CCL4L2, ANXA5, HNMT, CXCL2, AOAH, CD163, LAP3, SERPINF1, TYMP,
SCPEP1, ATP6AP2, MFSD1, CCL3L3, LAMP2, HES1, RAC1, CLEC7A, TGFBI, HEXB, FPR3, AKR1B1, LGALS1, SGK1, SDCBP,
ATP6V0B, CD86, BLVRB, PLEK, AP2S1, CXCL16, IFI30, LGALS2, VAMP5, IFNGR1, GPR34, UBE2D1, MGST2, OAZ2,
C3AR1, PYCARD, MARCKS, NR1H3, HEXA, ATP6V1B2, NINJ1, HCK, RGS10, SLC7A7, P2RY13, NPL, IL4I1, BLVRA,
TSPAN4, STAB1, MAFB, SMCO4, CD36, NAGK, TPP1, SLAMF8, SAMHD1, FCGR1A, SLCO2B1, TFRC, TCN2, TMSB4X,
MNDA, TBXAS1, CECR1, HMOX1, FCGR2A, CD4, GM2A, RENBP, TMSB10, CD209, PILRA, PLTP, ACP2, NAAA, LILRB5,
KCNMA1, VMO1, CD300A, SCIMP, LPAR6, B2M, ABHD12, CLEC4A, KCTD12, CFD, NAIP, PTGS2, LAIR1, MPP1, CLEC4E,
C5AR1, RASSF4, ADAP2, MSR1, RAB20, SLC31A2, LACC1, TFEC, TIMP2, GNS, CSF2RA, SLC8A1, ABCA1, DAPK1,
CLEC12A, CD300LF, PTAFR, DMXL2, CMKLR1, CD302, GNPDA1, HLA-DQA2, RAB32, IFNGR2, CEBPD, SLC15A3, RAB31,
SIRPA, LITAF, LILRB2, CD163L1, ATP6AP1, SCARB2, SDS, LGALS3, AKR1A1, NCOA4, CH17-340M24.3, STAT1, LGALS9,
GPX4, SNX10, DNAJC5B, PLEKHO1, PLBD1, ALDH1A1, HSD17B14, EBI3, TNFAIP8L2, RB1, DNASE2, SGPL1, SLC38A6,
ZEB2, LINC00996, HLA-DOA, CSTB, NAMPT, CCL4, GATM, GPR137B, IL18BP, LY86, SIGLEC7, PTPRE, STX11, ADGRE2,
PLIN2, LAMP1, HBEGF, RNF13, CXCL10, CREBL2, GAA, AHR, LGALS3BP, ITPR2, SDSL, TLR4, IER3, SMPDL3A, RGS18,
ARRB2, TNFAIP2, SIGLEC10, FAM105A, PRCP, P2RY6, RGL1, TTYH3, M6PR, GSTO1, IGF1, CD33, CD84, NCF2, SRGN,
KYNU, TIMP1, MMP14, GLA, CHMP1B, ATP6V0E1, EFHD2, NCEH1, LRRC25, LINC00998, A2M, ICAM1, RBM47, NAGA,
NCF4, IDH1, CD81, IL13RA1, ME1, SPRED1, OTOA, GSTP1, GIMAP4, MAF, LPCAT2, HAVCR2, CFP, GRINA, PLXDC2,
BST2, SASH1, CCR1, HCST, DRAM2, ADORA3, GNB4, NRP2, SLC31A1, APLP2, CYP27A1, TXN, YBX3, SIGLEC1, RAB42,
QKI, CYFIP1, ZFYVE16, 1-Mar, FRMD4B, ARHGAP18, KLF4, PHACTR1, FUOM, CPM, CCRL2, PTMS, ATP6V1A, SDC3,
ATF3, CLTA, LACTB, TIFAB, H2AFY, HLA-A, THEMIS2, PTGDS, ZNF385A, SLC16A3, PLSCR1, CXCL12, MMP19, TMEM37,
SMS, SLC43A2, IL2RA, DRAM1, SLAMF7, POMP, NCKAP1L, HLA-B, EPB41L3, NRP1, IFITM3, PLEKHB2, OAZ1, LCP2,
MGAT1, FNIP2, UBE2E2, LYN, ATF5, TUBA1B, LAMTOR2, FCHO2, C10orf54, GNAI2, SH2B3, DPYSL2, CUL9, BCL2A1,
YBX1, FAM213A, ARPC1B, ANXA2, C10orf11, TKT, RRAGD, IRF8, SERPINA1, ALDH2, UNC93B1, MILR1, SPG21, NR4A3,
GSN, APH1B, TALDO1, GUSB, BCAP31, ARPC5, SYNGR2, C1orf162, CLIC2, CAPZA2, EPHX1, ACER3, WWP1, RNH1,
RNF149, PSME2, IQGAP2, ATP5E, GCA, TFPT, FEZ2, LILRB1, MCOLN1, PMP22, DOK2, ATP6V1C1, DBI, PLXNC1, PGD,
FAM110A, SDHB, ATP1B3, BID, CAMK1, COMT, YWHAH, CASP1, GBP1, FYB, GLB1, ATP6V0D1, INSIG1, MOB1A,
VOPP1, BNIP3L, HLA-C, PVRL2, STX4, TLR1, SHTN1, MKNK1, ARL6IP1, SCD, TNFRSF1A, HSPB1, CAPZB, TLN1, FUCA2,
CCDC88A, ATP1B1, PCBD1, ABI3, ARL5B, LRPAP1, CMTM6, ARPC3, SPATS2L, SOAT1, OTUD1, SPINT2, CD53, C15orf48,
ANKRD22, CANX, ATG7, BMP2K, TPMT, SH3BGRL, CXCL3, SERF2, ATP13A2, DPP7, PAK1, NFE2L2, RHOG, CLTC, NADK,
PHACTR2, NCF1, GLRX, ZFAND5, H2AFJ, RTN4, LIMS1, NDFIP1, PPIF, LAPTM5, RAP2B, PEPD, WARS, PRNP, CAT,
GADD45B, HN1, PLAU, FERMT3, SNX2, FAM49B, RHOA, FGD2, AP1S2, EPSTI1, LCP1, HLA-DQB2, CPPED1, SERPINB6,
FKBP1A, RAB10, CYB561A3, SPPL2A, MGST3, CISD2, SNAP23, SNX6, TCEAL4, CBR1, MOSPD2, SQRDL, RAB5C, NFKBIE,
RAB7A, LAMTOR1, CEBPB, GLMP, NUPR1, CD59, ARL6IP5, PDK4, C4orf48, RNPEP, ACTB, SNAP29, SEC11A, PRDM1,
AKIRIN2, LAMTOR4, UCP2, IL1RN, CORO1B, CNPY3, ZDHHC4, PDXK, TM9SF2, LAPTM4A, ATG4C, CALM2, EPB41L2,
TMEM138, NAA20, DNPH1, TWF2, DHRS3, MSRB2, PHLDA2, RSU1, NENF, RAB9A, SKAP2, SULT1A1, FAM96A,
TMBIM6, PRDX3, VMA21, TMEM219, ATG3, GRB2, HSBP1, QPRT, CPQ, LAG3, FBP1, GSTM4, EVI2B, NEU1, C20orf27,
ERP44, SLC25A5, AP2M1, MGLL, PTTG1IP, SFT2D1, NDUFB5, VIMP, PKM, HMGN3, VPS29, RGS2, TIMM8B, BLOC1S1,
NDUFA12, PARL, HCLS1, SMIM4
CD.Mac.C1QB.CD14
FCGR1A, VSIG4, FCN1, C5AR1, CD14, IGSF6, IL1B, MNDA, CPVL, MS4A7, P2RY13, CLEC10A, C1QC, MS4A6A, MRC1,
CXCL8, PLAUR, CXCL2, PTGS2, FGL2, OLR1, SLAMF8, CD36, FCGR3A, C1QB, AOAH, CSF1R, CLEC7A, FAM26F, C1QA,
CD302, LST1, MSR1, CD163, AIF1, CD68, LILRB2, SLC7A7, CYBB, C3AR1, MS4A4A, FCER1G
CD.Mac.CXCL3.APOC1
MS4A4A, CCL3L3, CD163, GPNMB, FOLR2, APOC1, MRC1, MSR1, CXCL3, CCL4L2, MS4A7, MS4A6A, CXCL2, TNF,
FCGR3A, CD14, SLC40A1, CXCL8, CCL3, IL1B, PTGS2, CPVL, C1QC, FCGR1A, FGL2, CD209, NPL, C1QB, LGMN, FPR3,
APOE, IGSF6, C1QA, CYBB, ABCA1, STAB1, C3AR1, CCL4, PLA2G7, C5AR1, FCER1G, TTYH3, CTSL, LIPA, SEPP1, CD68
CD.Mac/DC.CXCL10.CLEC4E
CXCL10, CXCL9, IGSF6, PLA2G7, CLEC4E, P2RY13, FCGR1A, C1QC, TFEC, CLEC10A, C1QB, FAM26F, IL4I1, SLAMF8,
FGL2, TNFAIP6, PLAUR, CYBB, CD68, GBP1, C1QA, INHBA, MNDA, FCER1G, RP11-1143G9.4, CPVL, CD86, AIF1,
TYROBP, IL1RN, PLEK, LST1, IL1B, MMP9, MPEG1, CLEC7A, FCGR1B, LYZ, SOD2, PILRA, IDO1, FPR3, LILRB4, CLEC4A,
IFI30, TMEM176B, TNFSF13B, LGALS2, C1orf54, NR1H3, TLR8, CXCL11, SPI1, SNX10, CXCL8, CTSB, SLC8A1, HCAR3,
HCK, TNFAIP2, KCNMA1, LPCAT2, ATP6V1B2, WARS, ANKRD22, P2RY6, GBP5, LAMP3, SLC7A7, MS4A7, APOC1, FPR1,
CCL3, SLC31A2, RP11-196G11.2, CLEC6A, AOAH, SCIMP, JAML, HLA-DQA1, TFRC, CSF1R, ENPP2, APOL4, GLUL, CTSS,
SLAMF7, HLA-DRB1, LILRB2, CTSZ, C3AR1, PSAP, MRC1, CD14, LAP3, CSTA, RNF130, CFP, SMCO4, KYNU, LIPA, HCAR2,
SGK1, CMKLR1, LY96, GRN, DMXL2, CEACAM4, CCL19, TMEM176A, TXN, ACP5, HNMT, STAT1, UBE2D1, CYP27B1,
HLA-DRA, NAMPT, EBI3, HLA-DPB1, SAMHD1, CST3, HLA-DMA, NPC2, TYMP, FTL, CTSH, PTPRE, FCGRT, CTSC, HLA-
DPA1
CD.Mcell.CSRP2.SPIB
GJB3, RHOV, LECT1, NOXO1, WIPF3, PRKCG, CSRP2, FERMT1, LYPD8, TINAG, C6orf141, CCL15, IGFBP2, PTPRF, IL17RB,
SDR16C5, GABRP, FAM101A, EFHC2, NTRK2, OTC, PPP1R1B, HOXB9, KCNK1, PKP2, SFN, GOLM1, ATP2C2, ARL14,
GIPC2, LAD1, TNFRSF11A, UGT2A3, EDN1, RP11-357H14.17, AQP1, CDX1, DSG2, SLC44A3, S100A14, DSC2, KLF5,
RAB25, UBD, KREMEN1, KIAA1324, MGST1, ABO, LINC00675, STAP2, TRIM2, CHDH, KRTCAP3, ST6GALNAC1,
CKMT1A, CCL25, PTGR1, C10orf99, STXBP6, RP11-532F12.5, SERPINB5, HEPH, PDZK1IP1, DDC, CPS1, PLPP2, PLCB4,
GSTO2, CFTR, DHRS11, LAMB3, FAM3D, AKR1C3, CDH17, MYB, TMEM54, FAM3B, DGAT2, ALDH7A1, CDC42EP4, DSP,
HMGCS2, H2AFY2, ST14, STARD10, FNBP1L, FABP2, ADH1C, SDC4, PVRL1, GPRC5A, GPX2, EPS8, CCND1, USH1C,
PXMP2, AP1M2, PLEK2, SLC44A4, LSR, SOX9, HOXB7, C19orf33, ERBB3, TMPRSS2, MTMR11, TMEM98, TRIM16,
FXYD3, CBLC, CLDN7, CLDN15, GATA6, CHMP4C, SYNPO2, FAM84A, PHLDA2, EPHB2, SH3BGRL2, TOM1L1, NFIB,
KIAA1147, ANXA13, EPHA2, CCDC64B, CLDN4, MARVELD2, VIL1, AMOTL2, POF1B, ANKRD29, CMBL, AGR3, RNF43,
TMEM45B, SH2D4A, TST, PPP1R14D, CDH1, PPP1R9A, TRABD2A, MISP, HSD3B7, GJB2, ANKRD22, GJB1, CXADR,
KCNE3, TMPRSS4, ITGB8, DDAH1, GSTA1, MYO6, RNASE1, PERP, TMEM171, SLC27A2, PRAP1, CLDN3, HNF4A, CENPV,
TSPAN8, ERBB2, BACE2, CDHR1, HBEGF, TMEM97, TCEA3, CERS6, CKMT1B, CNN3
CD.Mono.CXCL10.TNF
CCL3, CCL3L3, IL1B, CXCL10, CCL4, CXCL8, CCL4L2, CXCL2, SOD2, TNF, CXCL3, BCL2A1, G0S2, CXCL9, PLAUR, S100A8,
IER3, S100A9, AIF1, PLEK, FCER1G, TYROBP, IL1RN, PTGS2, LST1, FCN1, TNFAIP6, TNFAIP2, FGL2, CYBB, CD14, MNDA,
MS4A7, SERPINA1, CCRL2, FCGR3A, FCGR1A, NLRP3, CLEC4E, ANKRD22, KCNJ2, KB-1507C5.4, IL1A, AQP9, IRG1,
GPR84, IGSF6, FAM26F, PILRA, NINJ1, RP11-1143G9.4, LYZ, LINC01272, FPR1, GBP1, CD300E, MIR3945HG, CLEC7A,
CPVL, MS4A6A, PSTPIP2, CSTA, LILRB2, SNX10, ICAM1, DRAM1, FCGR1B, IDO1, CD68, STX11, SPI1, TNFSF13B, MAFB,
GCH1, CTSB, SGK1, TYMP, PLA2G7, MPEG1, NAMPT, VAMP5, NFKBIA, EREG, KYNU, GK, CLEC4A, RP11-670E13.6,
FCGR2A, AC069363.1, CST3, TMEM176B, CXCL11, DMXL2, NFKBIZ, PSAP, NCF2, HCK, FFAR2, FPR2, ABL2, CSF1R,
IFITM3, FTL, NPC2, C5AR1, NRG1, WARS, S100A11, CTSS, HLA-DRB1, FTH1, C1QA, GLUL, LCP2, ATP6V1B2, ACSL1,
SERPINB9, CEACAM4, TLR2, PTPRE, CD274, IL27, SAT1, SMCO4, SLAMF8, TNFAIP3, DUSP1, C15orf48, IFIT2, LILRB3,
C1orf162, HLA-DRA, ZEB2-AS1, LINC00936, C1QC, SIRPA, ASGR1, PFKFB3, OLR1, C1QB, TMSB10, LAP3, ADGRE2,
CD36, RIPK2, LILRA5, INHBA, CD83, KCTD12, PLSCR1, TIMP1, TMEM176A, SRGN, DSE, RAB31, SLC7A7, SPHK1, FPR3,
APOBEC3A, LILRB4, HLA-DPB1, GBP5, AOAH, GCA, CD86, DUSP2, LITAF, CCL20, CFP, ARL5B, CYBA, TLR8, CLEC10A,
HLA-DPA1, FAM49A, P2RY13, HLA-DQB1, HLA-DMA, S100A12, HLA-DQA1, CLEC12A, CSF3R, GRN, GRINA, ZFP36,
SLC8A1, IL4I1, JAML, SLC31A2, GPX1, TFEC, RNF19B, MARCKS, TBXAS1, ZEB2, DUSP6, CDC42EP2, SCIMP, LCP1, CTSZ,
C3AR1, HCAR3, CD63, RAPGEF2, SAMHD1, SLC43A2, NEAT1, LY96, CSF2RB, LILRB1, VAMP8, HAPLN3, EGR1,
MIR155HG, RNF130, LYN, POMP, ITGB2, PYCARD, PNRC1, NR4A3, PPIF, MXD1, PIM3, LRRK2, PPT1, ATF5, CTSC, TPP1,
CMTM6, CTSH, FGR, PSME2, CMKLR1, FCGRT, PRDM1, NCOA4, ATOX1, STAT1, CD74, PIK3AP1, SECTM1, CASP1, CCR1,
SIGLEC12, GBP2, MS4A4A, AP2S1, ANXA5
CD.Mono.CXCL3.FCN1
CXCL8, IL1B, CCL3, CXCL2, CXCL3, CCL3L3, PLAUR, LST1, FGL2, EREG, PTGS2, FCN1, IGSF6, MNDA, CPVL, CLEC7A, AIF1,
MS4A6A, NLRP3, FCGR1A, CSTA, IL1RN, CSF1R, BCL2A1, OLR1, CLEC10A, TYROBP, CYBB, FCER1G, RP11-1143G9.4,
LILRB2, TNF, PILRA, MS4A7, CD68, MPEG1, C1QC, CCL4L2, PLEK, CCL4, IL1A, C1QA, SOD2, IER3, LYZ, FAM26F, C1QB,
CD14, C5AR1, CD36, JAML, SPI1, FCGR3A, PLA2G7, MIR3945HG, SLC7A7, CLEC4A, CD86, TNFAIP6, TNFSF13B,
TNFAIP2, SAMHD1, NAMPT, VSIG4, HCK, CLEC12A, AOAH, FPR3, STX11, CST3, LIPA, GLUL, CCRL2, RNF130, NPC2,
SERPINA1, SGK1, C1orf162, LILRA5, CTSS, C3AR1, CFP, NRG1, TBXAS1, MRC1, TIMP1, CTSB, PSAP, ASGR1, ADGRE2,
MAFB, TGFBI, P2RY6, HLA-DRB1, P2RY13, ICAM1, SLC8A1, SCIMP, CDC42EP2, GRN, NCF2, HLA-DMA, G0S2, RNASE6,
FTL, LINC01272, KCNJ2, TMEM176B, FCGR1B, PTPRE, HLA-DPB1, HLA-DRA, S100A11, RGS18, TYMP, SMCO4, ARL5B,
SLAMF8, GPX1, GPR84, KCTD12, HNMT, PYCARD, SAT1, FCGRT, ATP6V1B2, LGALS2, PPT1, STAB1, S100A9, HLA-DQA1,
CD83, NFKBIA, NAIP, HLA-DPA1, FTH1, DMXL2, CD209, CD1D, KLF4, CECR1, HLA-DMB, KB-1507C5.4, SIGLEC12,
RAB20, VAMP5, NINJ1, GCA, FCGR2A, TREM1, HLA-DQB1, ASAH1, SRGN, CLEC4E, NFKBIZ, TMEM176A, CD300A, TLR4,
ZEB2-AS1, PRDM1, ANKRD22, CXCL10, TFEC, TLR8, CYBA, ITGB2, FPR1, CTSZ, CD302, VAMP8, DUSP1, GBP1, LAP3,
MS4A4A, GCH1, CASP1, KYNU, NR4A3, IFITM3, LY96, SNX10, HCAR3, LPCAT2, LILRB4, RAB31, TLR2, PLBD1, SLC31A2,
PTX3, RAB32, WARS, LILRB3, CSF3R, CD300LF, DAPK1, ZEB2, PLSCR1, EMILIN2, BID, TNFAIP3, RIPK2, CTSC, TMSB10,
CD74, NAAA, CEACAM4, OTUD1
CD.Mono.FCN1.S100A4
AIF1, LST1, TYROBP, LYZ, FCN1, S100A9, MNDA, FTL, FCER1G, S100A8, FGL2, IL1B, PLAUR, CYBB, IGSF6, MS4A6A,
CST3, EREG, RP11-1143G9.4, OLR1, SOD2, FTH1, TMSB10, FAM26F, CTSS, CSTA, SAT1, NPC2, NAMPT, S100A11, HLA-
DRA, HLA-DRB1, CLEC7A, CPVL, PSAP, CD36, CYBA, JAML, TMSB4X, TIMP1, SERPINA1, CD14, GPX1, G0S2, SAMHD1,
C1orf162, VAMP5, TYMP, NRG1, HLA-DPB1, PTGS2, SPI1, SRGN, MPEG1, LGALS2, LINC01272, TNFSF13B, FCGR1A,
S100A4, C5AR1, ACTB, PLEK, ATP5E, ASGR1, BCL2A1, IFI30, PTPRE, PSME2, CLEC4A, PILRA, VAMP8, PYCARD, TREM1,
B2M, HLA-DPA1, GLUL, MS4A7, SERF2, KCTD12, SH3BGRL3, PLA2G7, CDC42EP2, C1QA, LCP1, POMP, LAP3, CD68,
FPR1, GBP1, SCIMP, NCF2, OAZ1, CFP, WARS, SLC8A1, NEAT1, SMCO4, CD74, PRELID1, LILRA5, DMXL2, CXCL8,
CLEC12A, TMEM176B, HBEGF, KLF4, SGK1, HLA-DMA, CTSB, HCK, FCGR3A, LILRB2, IFITM3, CSF1R, LGALS1, H3F3A
CD.Mono/Mac.CXCL10.CXCL11
CXCL10, CXCL9, ANKRD22, PLA2G7, LILRB2, FCGR1A, CLEC4E, FCGR3A, CXCL11, IGSF6, PILRA, CCL3L3, CCL3, IL1RN,
IL1B, LILRB4, CSF1R, SLAMF8, CLEC10A, IDO1, C1QC, SIRPA, AIF1, LILRB3, IL27, P2RY13, FPR1, CLEC4A, CXCL8, CSTA,
IL4I1, CXCL2, GPR84, NR1H3, C1QB, RP11-1143G9.4, C1QA, CD68, CYBB, FAM26F, FCER1G, CD274, DRAM1, CD163,
PLEK, S100A9, GBP1, SNX10, PSTPIP2, LST1, FGL2, HCK, CPVL, SMCO4, TYROBP, FCGR1B, CD14, MRC1, FPR3, SOD2,
CMKLR1, GLUL, CLEC7A, SERPINA1, MNDA, FCGR2A, KYNU, RGL1, CCL4, SIGLEC1, KCNMA1, AOAH, LYZ, CSF2RB,
MPEG1, CD86, SLC2A6, PLAUR, ENPP2, TNFAIP2, TNF, TNFSF13B, TMEM176B, SLC31A2, SCIMP, NINJ1, MMP9,
SERPING1, HAPLN3, HCAR3, TMEM176A, GBP5, TNFAIP6, PTAFR, CTSB, MS4A6A, C3AR1, GCH1, CD300LF, CEACAM4,
SLC7A7, IER3, ATP6V1B2, NCF2, WARS, CCL19, CXCL3, TFEC, LAIR1, LILRB1, CCL4L2
CD.Mono/Mac.CXCL10.FCN1
CXCL10, CXCL9, FCN1, S100A9, S100A8, FCGR1A, LILRA5, IL1B, GBP1, LINC01272, RP11-1143G9.4, AIF1, FGL2, MNDA,
ANKRD22, FPR1, PLAUR, IDO1, CYBB, FCER1G, LST1, TYROBP, CD14, CXCL11, IGSF6, PLEK, SOD2, SERPINA1, G0S2,
LYZ, FCGR3A, CLEC4E, PLA2G7, KCNJ2, GK, CPVL, FPR2, GPR84, WARS, FAM26F, TNFAIP2, IL1RN, FCGR1B, LILRB2,
CLEC7A, TNFSF13B, VAMP5, P2RY13, HCK, PSTPIP2, BCL2A1, CCL3, GCH1, TIMP1, CSF1R, NAMPT, SLAMF8, DRAM1,
CDC42EP2, ATP6V1B2, FAM49A, CEACAM4, CSTA, CD300E, TYMP, PTGS2, CLEC4A, C15orf48, APOBEC3A, SNX10,
DSE, MPEG1, CFP, SLC8A1, GLUL, LAP3, STX11, CD36, OLR1, SERPINB9, KCTD12, PILRA, IL4I1, SCIMP, SMCO4, CD68,
FCGR2A, SPI1, SLC43A2, GBP5, C5AR1, CTSS, PTAFR, PLSCR1
CD.NK.CCL3.CD160
NKG7, GZMA, CCL4, CMC1, KLRD1, TRDC, KLRF1, CCL5, GZMB, XCL1, CD160, XCL2, IL2RB, CCL3, AREG, KLRB1, CLIC3,
CST7, KLRC1, FCER1G, GZMK, CTSW, CD7, B3GNT7, IFITM2, PRF1, HOPX, PIK3R1, TYROBP, NR4A2, GZMM, ALOX5AP,
MATK, HCST, SRGN, SH2D1A, TMIGD2, KRT86, FASLG, CLDND1, CD247, TXK, TMSB4X, DUSP2, SYTL3, PFN1, SAMD3,
ID2, GPR65, H3F3B, TNFSF14, PYHIN1, SH2D1B, CD69, KRT81, EOMES, IFITM1, MYL12A, PTGDR, EVL, CXCR4, GNLY,
7-Sep, GZMH, CD96, REL, CCL4L2, ZNF331, TRGC1, RNF125, BCO2, ZFP36, BTG1, STAT4, NCAM1, MAP3K8, STK17A,
PTPRC, GATA3, B2M, VPS37B, UBB, AOAH, APMAP, CORO1A, TMEM2, FGR, FAM177A1, GSTP1, FOSL2, HSP90AA1,
FYN, CREM, SRSF2, TRBC1, APOBEC3G, SLFN13, ACTB, IER2, HLA-A, IFNG, LCP1, ELF1, CHST12, RORA, CLEC2B, RHOC,
MCTP2, PRKCH, MAPK1, RGCC, YPEL5, HSP90AB1, JAK1, PDCD4, AKNA, SKAP1, PTPN22, TIPARP, ARAP2, EIF3G,
CALM1, PIP4K2A, LY6E, PTPN4, DDX5, IRF1, HELZ, RARRES3, SACM1L
CD.NK.GNLY.FCER1G
GNLY, GZMA, GZMB, NKG7, CMC1, KLRD1, TRDC, KLRF1, KLRC1, IL2RB, GZMK, GZMH, PRF1, KLRB1, FCER1G, XCL1,
TYROBP, CCL5, CST7, CTSW, SH2D1B, B3GNT7, AREG, XCL2, CLIC3, CCL4, KRT81, KRT86, HOPX, TMIGD2, MATK,
IFITM2, SRGN, CD7, KIR2DL4, SAMD3, NCAM1, HCST, EOMES, TXK, CCL3, CD160, BCO2, ADGRG3, GPR65, CLDND1,
ID2
CD.NK.GNLY.GZMB
GNLY, GZMB, FGFBP2, PRF1, KLRF1, KLRD1, CTSW, NKG7, MYOM2, CX3CR1, GZMA, S1PR5, GZMH, SPON2, FCGR3A,
CCL4, CST7
CD.NK.GNLY.IFNG
GNLY, KLRD1, KLRF1, XCL1, KLRC1, TRDC, XCL2, IFNG, GZMB, NKG7, GZMA, CTSW, PRF1, CMC1, HOPX, CCL4, GZMK,
IL2RB, TYROBP, CST7, FCER1G, MATK, DUSP2, CLIC3, CD69, CD7, SH2D1B, KLRB1
CD.NK.MKI67.GZMA
TRDC, MKI67, KLRD1, TOP2A, UBE2C, CENPF, ASPM, NUSAP1, TYMS, KLRF1, AURKB, XCL2, KLRC1, XCL1, CDK1, KIF11,
EOMES, KIF23, ZWINT, GZMB, KIF15, SPC25, RRM2, HIST1H1B, NDC80, TPX2, CKAP2L, IL2RB, CASC5, KIAA0101,
CENPE, BUB1, KIR2DL4, CDCA8, CENPU, BIRC5, GZMA, DLGAP5, NKG7, HIST1H1D, NCAPG, CTSW, SLFN13, SH2D1A,
FBXO43, MAD2L1, SMC2, GTSE1, B3GNT7, SH2D1B, CCNA2, FAM64A, NUF2, CDCA2, KIF2C, SAMD3, ATAD2, STMN1,
SKA3, ADGRG3, CDKN3, CMC1, CENPK, TK1, RAD51AP1, HJURP, HMMR, NCAPG2, PRC1, ASF1B, KIF14, MATK, TRGC1,
TNFSF14, CKS1B, UHRF1, GZMH, HIST2H2AC, ESCO2, SMC4, CD160, TRGV9, S1PR5, CENPA, EZH2, SGOL2, CDCA3,
KIF4A, PRF1, CENPN, NCAM1, SPC24, TROAP, HIST1H1E, KIFC1, DIAPH3, CDCA5, CDT1, CDC20, NEK2, CEP55, CLSPN,
UBE2T, HIST1H3G, ORC6, LMNB1, HMGB2, CENPM, POLQ, KRT86, PCNA, FASLG, ANLN, PLK1, GNLY, FAM111B,
PTTG1, TXK, HIST1H2AJ, PTGDR, ATAD5, DTYMK, GGH, CDKN2D, RAD51, FBXO5, OIP5, PLK4, CHEK1, GMNN, CLDND1,
TMIGD2, DNAJC9, PBK, KIAA1524, CCL4, CLIC3, SHCBP1, MND1, ECT2, TUBB, CCNB2, LRR1, PKMYT1, TYROBP,
FCER1G, FEN1, KRT81, NCAPD3, CENPW, KIF20B, DEPDC1B, RACGAP1, APOBEC3H, STIL, C12orf75
CD.Paneth.DEFA6.ITLN2
DEFA5, REG3A, DEFA6, PRSS2, PLA2G2A, ITLN2, LYZ, GUCA2A, SERPINA1, LCN2, REG1A, REG1B, TFF3, RP11-
1143G9.4, IGFBP2, HES6, GPX2, SPINK1, KLK12, RETNLB, CH17-125A10.2, CDKN1C, PCSK1, AZGP1, TM4SF5, PDIA2,
PLCG2, ELF3, PRUNE2, STARD10, MUC1, CLCA1, SCNN1A, S100A1, SMIM22, SLC39A5, KRT18, SYP, KLK11, RAB26,
HEPACAM2, REP15, CDHR5, HOOK2, MT1G, RRBP1, PLEKHB1, RGMB, MFI2, PCLO, ARHGEF38, GADD45G, SMOC2,
IFI6, MT-CYB, SHC2, PROM1, ATOH1, FXYD3, MST1, EPHB2, LINC00261, MT-ND2, CLDN4, MTRNR2L12, CRACR2A,
MT-ND4L, DLL4, ENTPD8, REEP6, BCAS1, AOC1, NOXO1, PROX1, MTRNR2L8, MT-ND1, MT-ND4, MT-ND5, KIF19,
SPDEF, TMC5, DMBT1, MT-CO1, KIAA1324, MT-ATP6, MT-CO3, MT-CO2, GALNT5, MUC13, KCNQ1OT1, COL16A1,
CCSER1, EHF, PTP4A3, EPHB3, FOXA3, SEC61A1, MT-ND3, BACE2, RASD1, HID1, ERN2, BAIAP2L2, AFMID, GOLGA8A,
SOX6, CAMK2N1, TCF7L2, SOX9
CD.pDC.IRF7.IL3RA
GZMB, IRF7, LILRA4, PLD4, C12orf75, CLIC3, UGCG, IL3RA, IRF4, SERPINF1, SCT, PTPRS, PTCRA, SMPD3, MYBL2,
LILRB4, MAP1A, LRRC26, CLEC4C, SUSD1, LAMP5, KRT5, RP11-73G16.2, EPHB1, PACSIN1, RNASE6, TCF4, PHEX,
PPP1R14B, PLAC8, TNFRSF21, TSPAN13, BCL11A, CCDC50, SPIB, EGLN3, SCN9A, IRF8, ALOX5AP, ZFAT, TRAF4, GNA15,
SLC15A4, P2RY14, LDLRAD4, PROC, FAM129C, AC011893.3, SMIM5, C1orf186, TMEM8B, RPS6KA4, RP11-117D22.2,
MS4A6A, NME8, PLEK, ASIP, CSF2RB, TPM2, LINC00996, NEK8, TYROBP, OPN3, APP, PPM1J, SELL, RUNX2, PTPRE,
MPEG1, FCER1G, DNASE1L3, SLC20A1, SOX4, CHAF1A, IDH3A, GPR183, THBD, SHD, KCNA5, GAS6, ITM2C, CYB561A3,
SIRPB1, STMN1, JAML, INPP4A, SEC61B, P2RY6, ARL4C, ATP13A2, LINC00865, GAPT, FAM213A, FUZ, SCAMP5,
ZBTB18, NR4A3, RAB11FIP1, POLB, CXCR3, CD4, THEMIS2, HIGD1A, PLP2, CCDC88A, GRN, RASSF2, SLC7A5, ABHD15,
TXN, ZC3HAV1, MAPKAPK2, NPC2, CORO1C, SLC7A11, ST3GAL4, CHML, ATF5, PTGDS, CST3, RUBCN, CNPY3, GPX1,
CD74, VAMP8, SELPLG, FADS1, C3orf58, CYBB, N4BP2L1, SFT2D2, SAMHD1, RGS2, RNASET2, SIDT1, KLF4, CAPG,
MAP3K2, FCHSD2, N4BP2, BID, HS3ST3B1, PRMT9, MZB1, AP1S2, SNCA, VIMP, SH2B3, SPCS1, TUBB6, PRKCB, MGST2,
ADA, PHACTR1, CTSC, TGFBI, NR3C1, FZD3, PARK7, ZDHHC17
CD.T.CARD16.GB2
IL32, RGS1, CD3D, B2M, TRAC, RTKN2, S100A4, TIGIT, PTPRC, TRBC2, FOXP3, CD2, MALAT1, MIR4435-2HG, ARID5B,
BATF, CXCR6, TBC1D4, CTLA4, TRBC1, LTB, LAIR2, CD3E, AC133644.2, ICOS, IL2RA, RPS26, HLA-A, LINC00152, ACTB,
LCK, PYHIN1, AC017002.1, BTG1, TNFRSF4, CD7, ARHGDIB, RPS27, TNFRSF18, GBP2, FYB, GBP5, CARD16, DUSP4,
IL1R2, CD27, CTSC, SLA, METTL8, SIRPG, TNFRSF9, SAMSN1, PBXIP1, CD28, UGP2, LAT, CD3G, BIRC3, TNIP3, SRGN
CD.T.CCL20.RORA
KLRB1, S100A4, CD3D, CD2, AC092580.4, RORA, IL7R, IL32, CD52, PTPRC, TRAC, B2M, TMSB4X, SH3BGRL3, CCL5,
GZMA, CD3E, ALOX5AP, CD96, TRAT1, IFNG, CD69, GPR171, LTB, CHN1, TNFAIP3, PFN1, TRBC2, CCR6, CKLF, ID2,
CD3G, SPOCK2, RPLP1, SRGN, FYB, CXCR6, ACTB, CCL20, LCK, CD40LG, TRBC1, EVL, CREM, IL2RG, LAT, CLEC2D,
ABRACL, GIMAP7, HLA-A, ANXA1, MGAT4A, RBL2, MYL12A, RARRES3, TNFRSF25, THEMIS, PTPN22, CORO1A, PDE4D,
BTG1, TANK, RGS1, PYHIN1, ARHGDIB, LEPROTL1, ITM2A, CD6, CST7, RPS19, STK17B, SAMSN1, GBP5, CTLA4, GRAP2,
JAML, GMFG
CD.T.GNLY.CSF2
GNLY, GZMB, GZMA, IFNG, PRF1, AC092580.4, CD2, IL26, S100A4, CCL5, CD3D, CXCR6, GZMH, HOPX, CSF2, IL32,
ALOX5AP, CD52
CD.T.IFI44L.PTGER4
IL7R, TNFAIP3, KLRB1, CD69, ANXA1, TSC22D3, TRAC, CD3D, CD2, CD3E, TRAT1, S100A4, KLF6, AC020571.3, GPR183,
IFI44L, TRBC2, ICOS, RGS1, IL32, RPLP1, HLA-B, GPR171, CXCR4, CD3G, B2M, RORA, TMSB4X, LCK, ZFP36L2, PTGER4,
BTG1, CD40LG, MYADM, CCL5, CD6, DDIT4, TRBC1, CLEC2D, SARAF, CREM, SRGN, JUNB, EVL, LEPROTL1, YPEL5, FOS,
MYL12A, CD7, GATA3, FYB, LY6E, SYTL3, ID2, SAMSN1, CRIP1, SPOCK2, LAT, NFKBIA, GIMAP7, CD96, EIF1, IFITM2,
DDX5, AC092580.4, DUSP2, SH2D2A, TNFRSF25, HLA-A, ACTB, TNFSF13B, H3F3B, PHLDA1, DUSP1, RPS29, CCR6,
FTH1, ITM2A, FLT3LG, ODF2L, RGL4, CORO1A, ISG15, ACAP1, RGCC, ZFP36, HLA-C, PPP1R15A, RPL10, CKLF, MCL1,
TTC39C, RPLP2, STAT4, CD247, CALM1, RPS12, DNAJB1, HLA-E, HCST, ARHGDIB, TC2N, SOCS1, LTB, ALOX5AP, ITM2B,
PPP2R5C, SCML4
CD.T.IFI6.IRF7
IFI44L, ICA1, TIGIT, ISG15, CD3D, MX1, TRAC, CD2, CPA5, AC133644.2, IFI6, SCGB3A1, LY6E, ICOS, HLA-A, SPOCK2,
IL32, CD3E, TBC1D4, SIRPG, PBXIP1, CD7, LAG3, HLA-B, ISG20, TRBC2, LAIR2, LAT, TNFRSF4, DUSP4, TNFRSF1B
CD.T.LAG3.BATF
AC133644.2, TBC1D4, BATF, LAIR2, IL32, CD2, AC017002.1, LAG3, CD3D, FOXP3, TNFRSF1B, CXCR6, CTLA4, MIAT,
ADTRP, TIGIT, PBXIP1, TRAC, TNIP3, CD7, IL2RA, MALAT1, TRBC2, CD3E
CD.T.MAF.CTLA4
KLRB1, CD3D, CD2, CTLA4, IL32, RGS1, PTPRC, TBC1D4, PYHIN1, TRAC, TRBC2, RORA, MAF, B2M, CD3E, ICOS,
MIR4435-2HG, TRBC1, SRGN, S100A4, FYB, CCR6, METTL8, ARID5B
CD.T.MKI67.FOXP3
KIAA0101, TOP2A, CENPF, MKI67, NUSAP1, BIRC5, TYMS, CEP55, FOXP3, CTLA4, IL2RA, TPX2, TK1, NUF2, ASPM,
CENPA, GTSE1, MAD2L1, CDCA5, NDC80, DLGAP5, SGOL1, BATF, RRM2, KIF2C, CCNB2, CDK1, SKA1, ZWINT, CCNA2,
UBE2C, PTTG1, DEPDC1B, CENPE, RTKN2, CDKN3, STMN1, NCAPG, AURKB, IL1R2, CDCA8, PLK1, ASF1B, CDT1, CLSPN,
AC133644.2, HJURP, CDKN2A, PHF19, HMMR, KIF23, TNIP3, AC017002.1, AC002331.1, BUB1, CENPU, SMC2, CCNB1,
KIF11, RACGAP1, MTFR2, F5, CXCR6, SGOL2, CENPK, TRIB2, HMGB2, PRC1, TACC3, SPC25, SMC4, KIF14, UBE2T, KIFC1,
TNFRSF9, CD28, RMI2, TNFRSF18, IKZF2, ESCO2, CXCR3, CKS1B, MCM10, ICOS, SPC24, CASC5, MIR4435-2HG, KIF15,
EZH2, CENPM, SPOCK2, SIT1, DTL, ATAD2, NEK2, KIAA1524, HELLS, FEN1, PRR11, TIGIT, DNAJC9, NINJ2, ARHGAP11A,
CDCA2, CD70, BRCA1, TTK, DTYMK, KIF4A, LMNB1, PBK, UHRF1, PCNA, CDC20, CENPW, MELK, AURKA, TIMELESS,
NUDT1, DUSP4, CKAP2L, TMPO, DDB2, ORC6, SIRPG, LAIR2, FANCI, TROAP, MXD3, KIF20B, ANP32E, CENPN, PMVK,
SHCBP1, TCF19, MCM7, TNFRSF4, ATAD5, S100A4, FAS, TNFRSF1B, TUBB, DEPDC1, ICA1, GALM, SPDL1, CD2, CD3G,
RAD51, LAT, IL32, RPA3, CKAP2, RRM1, GBP5, HMGN2, FDXR, LINC00152, DDX39A, LCK, SKA2, TRAC, NCAPH, CDCA3,
GINS2, CALM3, TFDP1, FRMD4B, ZBTB32, DUT, H2AFZ, CD3D
CD.T.MKI67.IFNG
KIAA0101, TYMS, IL26, IFNG, RRM2, MKI67, TK1, ZWINT, CENPU, BIRC5, NMU, JAKMIP1, ADA, UHRF1, NUSAP1,
PKMYT1, GINS2, APOBEC3H, CENPF, CLSPN, CDKN3, GZMB, CDK1, SLC4A10, NUF2, MCM4, NCAPG, CEP55, MAD2L1,
TOP2A, KIF11, MCM10, C12orf75, GTSE1, DPP4, CD40LG, CDCA5, HOPX, STMN1, UBE2T, SMC2, ASPM, CXCR3, SPC24,
TESC, GZMA, MCM7, ADAM12, CDT1, CHN1, ATAD2, CENPM, FBXO5, EZH2, DTL, DSCC1, DHFR, FABP5, ASF1B, FEN1,
PCNA, CCNA2, AC092580.4, CDC6, SPC25, RRM1, CENPK, LMNB1, CDC45, HMGB2, TPX2, RAD51, MCM2, UBE2C,
PTTG1, DNAJC9, CSF2, CDC20, BATF, TMPO, S100A4, SLC27A2, FANCI, PBK, SMC4, CXCR6, TROAP, ANP32E, MCM3,
BRCA1, SLBP, CDCA7, KIF2C, MELK, HELLS, CHEK1, ZNRF1, ESCO2, DPP3, ACAT2, ALOX5AP, HJURP, IL12RB2, CD6,
GALM, SKA2, CKS1B, GNA15, MIR4435-2HG, COTL1, CDCA4, GZMH, MSC, CENPN, PHF19, SH2D1A, TNFRSF25,
GMNN, FLNA, DDX39A
CD.T.MKI67.IL22
TOP2A, NUSAP1, CENPF, MKI67, UBE2C, KIAA0101, RRM2, ASPM, CDK1, TYMS, BIRC5, CCNB2, DLGAP5, TPX2, ZWINT,
CCNA2, CDKN3, NUF2, GTSE1, HMMR, AURKB, CENPA, NDC80, KIF23, CEP55, NCAPG, IL26, BUB1, HJURP, SPC25,
KIF2C, CKAP2L, TTK, CDC20, KIF14, KIF11, CENPE, PRC1, MAD2L1, PLK1, CDCA8, CLSPN, IFNG, TROAP, RACGAP1,
KIFC1, SGOL2, CDCA2, CCNB1, CASC5, CDCA5, PBK, SMC2, DEPDC1, CDCA3, SMC4, KIF15, CKS1B, STMN1, ATAD2,
FBXO5, ASF1B, TK1, CENPU, KIF4A, HIST1H1B, NMU, CENPK, HIST1H1D, PTTG1, SGOL1, SKA3, DEPDC1B, NCAPH,
UBE2T, AURKA, CENPM, EZH2, SHCBP1, NEK2, ANLN, CD40LG, HIST2H2AC, HMGB2, MELK, TACC3, ESCO2, FAM83D,
C12orf75, RAD51AP1, AC092580.4, FANCI, PHF19, CDKN2D, ARHGAP11A, KIF20B, NCAPG2, SKA1, FBXO43, CENPN,
CENPW, DHFR, TMPO, CKAP2, PRR11, ANP32E, H2AFX, HIST1H2AJ, SPDL1, KIF22, IL22, APOBEC3H, GMNN, DTYMK,
ADAM12, E2F2, CDT1, DDX39A, C1orf112, CDC25B, GZMA, DIAPH3, TUBB, SLC4A10, USP1, FAM111A, STIL, KIF18A,
HIST1H1E, MTFR2, CCNF, RAD51, SPAG5, LMNB1, ZNF367, HIST1H2AH, HIST1H4C, KPNA2, MXD3, UHRF1, DNAJC9,
CCDC34, TRAT1, MCM10, BARD1, HN1, KIAA1524, TUBA1B, GPR171, SAE1, CD3G, GPSM2, SMC1A, POC1A, CCL20,
H2AFV, HMGB3, FEN1, BUB1B, CDC45, RFC4, RAD21, PCNA, CKAP5, CD2, RRM1, CD96, TUBA1C, NCAPD2, SKA2,
CHEK1, RORA, S100A4, UBE2S, BRCA1, HMGN2, ECT2, HIST1H2AM, FAM111B, DEK, NUDT1, CD6, CXCR3, ADA,
H2AFZ, CALM3, CD3D, GNG2, CKS2, DLEU2, KNSTRN, CXCR6

TABLE 2A
Differentially expressed genes in FR vs PR (Positive direction is enriched in FR and negative direction is enriched in PR).

| subset | gene | p_val | avg_logFC | pct.1 | pct.2 | p_val_adj | STAT |
|---|---|---|---|---|---|---|---|
| CD.Plsma/IgG.IGHGP.IGHG2 | IGHG3 | 1.67E−141 | −1.007367638 | 0.925 | 0.986 | 3.84E−137 | −2.62E+136 |
| CD.T.KLRB1.LTF | RPS4Y1 | 1.08E−126 | −1.372657797 | 0.05 | 0.555 | 2.48E−122 | −5.53E+121 |
| CD.T.CD8B.TRGC2 | RPS4Y1 | 1.28E−118 | −1.593820384 | 0.019 | 0.539 | 2.95E−114 | −5.40E+113 |
| CD.Plsma/IgM.IGHM.JCHAIN | EEF1A1 | 2.80E−106 | 1.011074667 | 0.993 | 0.975 | 6.44E−102 | 1.57E+101 |
| CD.Plsma/IgA.IGHA2.IGKV1-12 | EEF1A1 | 1.11E−94 | 1.083952129 | 0.992 | 0.975 | 2.55E−90 | 4.25E+89 |
| CD.B.MT-CO2.MT-CO1 | RPS4Y1 | 5.34E−92 | 1.500782623 | 0.44 | 0.028 | 1.23E−87 | 1.22E+87 |
| CD.NK.CCL3.CD160 | MALAT1 | 9.75E−88 | 0.488061252 | 1 | 0.998 | 2.24E−83 | 2.17E+82 |
| CD.Plsma/IgA.IGHA2.IGKV1-12 | MT-CO3 | 1.27E−86 | 0.906245261 | 0.977 | 0.943 | 2.91E−82 | 3.11E+81 |
| CD.Plsma/IgA.IGHA2.IGKV1-12 | TPT1 | 5.89E−86 | 1.032120985 | 0.989 | 0.978 | 1.36E−81 | 7.61E+80 |
| CD.Plsma/IgA.IGHA2.IGKV1-12 | RPL9 | 3.70E−83 | 0.979346259 | 0.981 | 0.916 | 8.51E−79 | 1.15E+78 |
| CD.T.CCR7.SELL | IGKC | 3.80E−83 | 0.639342524 | 0.973 | 0.905 | 8.74E−79 | 7.32E+77 |
| CD.Plsma/IgA.IGHA2.IGKV1-12 | MT-ND1 | 2.49E−82 | 1.010120374 | 0.968 | 0.857 | 5.74E−78 | 1.76E+77 |
| CD.Plsma/IgA.AL9287683.RPL39 | DEFA5 | 1.39E−80 | −1.040041015 | 0.122 | 0.67 | 3.21E−76 | −3.24E+75 |
| CD.T.CCR7.SELL | RPL10 | 3.17E−78 | 0.279258966 | 1 | 1 | 7.31E−74 | 3.82E+72 |
| CD.Plsma/IgA.IGHA2.IGKV1-12 | RPS3A | 1.51E−73 | 0.790832758 | 0.981 | 0.961 | 3.48E−69 | 2.27E+68 |
| CD.Plsma/IgA.IGHA2.IGKV1-12 | MT-ATP6 | 6.74E−72 | 0.870348632 | 0.953 | 0.852 | 1.55E−67 | 5.61E+66 |
| CD.Plsma/IgM.IGHM.JCHAIN | MT-ND1 | 1.19E−71 | 0.915437947 | 0.96 | 0.857 | 2.73E−67 | 3.35E+66 |
| CD.Plsma/IgG.IGHGP.IGHG2 | XIST | 3.26E−71 | −0.552331662 | 0.038 | 0.269 | 7.50E−67 | −7.36E+65 |
| CD.NK.CCL3.CD160 | TPT1 | 5.52E−71 | 0.777663147 | 0.949 | 0.851 | 1.27E−66 | 6.12E+65 |
| CD.T.CCR7.SELL | DEFA5 | 2.47E−70 | −1.592390476 | 0.071 | 0.37 | 5.68E−66 | −2.80E+65 |
| CD.B.MT-CO2.MT-CO1 | HLA-DRB5 | 3.25E−70 | 1.333920139 | 0.455 | 0.05 | 7.49E−66 | 1.78E+65 |
| CD.Plsma/IgA.IGHA2.IGKV1-12 | MT-ND2 | 9.21E−70 | 0.876058114 | 0.964 | 0.97 | 2.12E−65 | 4.13E+64 |
| CD.Plsma/IgA.IGHA1.IGKC | IGHG1 | 2.91E−68 | 1.119059079 | 0.746 | 0.371 | 6.69E−64 | 1.67E+63 |
| CD.Plsma/IgA.AL9287683.RPL39 | ZFP36 | 5.07E−68 | 0.804678267 | 0.78 | 0.234 | 1.17E−63 | 6.89E+62 |
| CD.NK.CCL3.CD160 | IGKC | 1.54E−66 | 0.762428673 | 0.976 | 0.877 | 3.55E−62 | 2.15E+61 |
| CD.NK.CCL3.CD160 | IGHA1 | 1.11E−64 | 0.668760475 | 0.952 | 0.788 | 2.55E−60 | 2.63E+59 |
| CD.Plsma/IgA.IGHA2.IGKV1-12 | MT-CYB | 5.43E−64 | 0.771786199 | 0.97 | 0.953 | 1.25E−59 | 6.18E+58 |
| CD.Plsma/IgM.IGHM.JCHAIN | TPT1 | 1.21E−62 | 0.795950484 | 0.973 | 0.951 | 2.80E−58 | 2.85E+57 |
| CD.Plsma/IgA.AL9287683.RPL39 | B2M | 2.20E−61 | −0.466218599 | 0.998 | 1 | 5.06E−57 | −9.21E+55 |
| CD.Plsma/IgA.IGHA2.IGKV1-12 | NACA | 5.55E−58 | 0.784678317 | 0.919 | 0.798 | 1.28E−53 | 6.14E+52 |
| CD.Bclls.CD69.GPR183 | MT-ND4L | 2.51E−57 | −1.513733267 | 0.263 | 0.75 | 5.77E−53 | −2.62E+52 |
| CD.Plsma/IgA.AL9287683.RPL39 | HLA-B | 7.72E−57 | −0.699052285 | 0.983 | 0.984 | 1.78E−52 | −3.94E+51 |
| CD.Plsma/IgM.IGHM.JCHAIN | RPL9 | 8.84E−57 | 0.650711773 | 0.955 | 0.9 | 2.04E−52 | 3.20E+51 |
| CD.B.IGHG1.IGLC2 | HLA-DRB5 | 2.35E−56 | −1.853103023 | 0.02 | 0.416 | 5.41E−52 | −3.42E+51 |
| CD.T.CCR7.SELL | IGHA1 | 6.71E−56 | 0.526438423 | 0.896 | 0.676 | 1.54E−51 | 3.41E+50 |
| CD.Plsma/IgM.IGHM.JCHAIN | MT-CO3 | 1.15E−55 | 0.690473348 | 0.985 | 0.943 | 2.64E−51 | 2.62E+50 |
| CD.B.MT-CO2.MT-CO1 | MT-ND3 | 6.09E−55 | 1.07712128 | 0.962 | 0.778 | 1.40E−50 | 7.68E+49 |
| CD.B.RPS29.RPS21 | RPS4Y1 | 4.41E−53 | −0.836233812 | 0.163 | 0.548 | 1.01E−48 | −8.25E+47 |
| CD.Plsma/IgA.IGHA2.IGKV1-12 | RPS27A | 4.68E−53 | 0.583470918 | 0.994 | 0.99 | 1.08E−48 | 5.41E+47 |
| CD.EC.MTRNR2L1.MT-ND3 | MTRNR2L8 | 2.18E−52 | 1.788231827 | 0.757 | 0.228 | 5.03E−48 | 3.56E+47 |
| CD.Plsma/IgA.AL9287683.RPL39 | HSPA1B | 5.67E−51 | 0.824788324 | 0.549 | 0.131 | 1.31E−46 | 6.32E+45 |
| CD.Plsma/IgM.IGHM.IGLV2-14 | EEF1A1 | 6.74E−51 | 1.02781615 | 0.981 | 0.94 | 1.55E−46 | 6.62E+45 |
| CD.Paneth.DEFA6.ITLN2 | MTRNR2L12 | 5.36E−50 | 1.840915527 | 0.834 | 0.165 | 1.23E−45 | 1.49E+45 |
| CD.Plsma/IgA.IGHA2.IGKV1-12 | MT-ND4 | 9.22E−50 | 0.592344517 | 0.972 | 0.985 | 2.12E−45 | 2.79E+44 |
| CD.Plsma/IgA.AL9287683.RPL39 | JCHAIN | 2.21E−49 | 0.450032758 | 1 | 1 | 5.08E−45 | 8.86E+43 |
| CD.Plsma/IgA.AL9287683.RPL39 | RPS12 | 4.01E−49 | −0.497117831 | 0.989 | 0.995 | 9.23E−45 | −5.38E+43 |
| CD.Mstcl.CTSG.CD69 | B2M | 5.92E−49 | −0.606417819 | 1 | 1 | 1.36E−44 | −4.45E+43 |
| CD.NK.CCL3.CD160 | EEF1A1 | 8.42E−49 | 0.50527135 | 0.964 | 0.93 | 1.94E−44 | 2.61E+43 |
| CD.T.CCR7.GPR183 | RPS26 | 1.04E−48 | 0.418606239 | 0.873 | 0.588 | 2.40E−44 | 1.75E+43 |
| CD.Plsma/IgA.IGHA2.IGKV1-12 | XIST | 1.77E−48 | −0.589688084 | 0.002 | 0.384 | 4.07E−44 | −1.45E+43 |
| CD.Plsma/IgM.IGHM.JCHAIN | MT-ND2 | 2.04E−48 | 0.67730957 | 0.978 | 0.988 | 4.69E−44 | 1.45E+43 |
| CD.Paneth.DEFA6.ITLN2 | MTRNR2L8 | 8.48E−48 | 1.757602626 | 0.8 | 0.13 | 1.95E−43 | 9.00E+42 |
| CD.T.CD8B.TRGC2 | RPS4X | 1.59E−47 | 0.396858057 | 0.988 | 0.981 | 3.66E−43 | 1.09E+42 |
| CD.Plsma/IgA.IGHA2.IGKV1-12 | RPL7 | 1.79E−47 | 0.583300233 | 0.975 | 0.966 | 4.13E−43 | 1.41E+42 |
| CD.T.KLRB1.LTF | IGHG4 | 6.48E−47 | −0.842724476 | 0.029 | 0.248 | 1.49E−42 | −5.65E+41 |
| CD.Plsma/IgA.AL9287683.RPL39 | RPLP1 | 9.45E−47 | −0.391098919 | 1 | 1 | 2.18E−42 | −1.80E+41 |
| CD.Plsma/IgG.IGHGP.IGHG2 | MT-ND4L | 1.85E−46 | −0.416273524 | 0.317 | 0.548 | 4.25E−42 | −9.80E+40 |
| CD.Plsma/IgA.AL9287683.RPL39 | MALAT1 | 9.39E−46 | −0.343833656 | 0.998 | 1 | 2.16E−41 | −1.59E+40 |
| CD.Plsma/IgA.AL9287683.RPL39 | RPL8 | 2.04E−45 | −0.417719715 | 0.985 | 0.99 | 4.69E−41 | −8.90E+39 |
| CD.Plsma/IgA.IGHA2.IGKV1-12 | IGLC2 | 2.13E−45 | 0.551716723 | 0.936 | 0.869 | 4.91E−41 | 1.12E+40 |
| CD.NK.CCL3.CD160 | IFITM1 | 2.44E−45 | 0.762641682 | 0.546 | 0.203 | 5.62E−41 | 1.36E+40 |
| CD.Plsma/IgM.IGHM.JCHAIN | MT-ATP6 | 1.14E−44 | 0.624700126 | 0.963 | 0.873 | 2.62E−40 | 2.38E+39 |
| CD.Plsma/IgA.IGHA2.IGKV1-12 | EEF1B2 | 1.64E−44 | 0.661412415 | 0.926 | 0.813 | 3.77E−40 | 1.76E+39 |
| CD.Plsma/IgA.AL9287683.RPL39 | REG1A | 4.64E−44 | −0.545619049 | 0.102 | 0.489 | 1.07E−39 | −5.11E+38 |
| CD.NK.CCL3.CD160 | IGHM | 1.19E−43 | 0.767968229 | 0.597 | 0.272 | 2.73E−39 | 2.81E+38 |
| CD.Plsma/IgM.IGHM.JCHAIN | RPS3A | 1.32E−43 | 0.526375312 | 0.967 | 0.953 | 3.04E−39 | 1.73E+38 |
| CD.EC.MTRNR2L1.MT-ND3 | IGHA1 | 3.83E−43 | 0.735700967 | 0.966 | 0.775 | 8.82E−39 | 8.34E+37 |
| CD.Mito.MT-CO2.MT-ND2 | HLA-DRB5 | 3.99E−43 | 1.170198487 | 0.587 | 0.198 | 9.19E−39 | 1.27E+38 |
| CD.Plsma/IgA.AL9287683.RPL39 | HLA-A | 6.79E−43 | −0.556796795 | 0.915 | 0.971 | 1.56E−38 | −3.56E+37 |
| CD.B.MT-CO2.MT-CO1 | MT-ND1 | 6.99E−43 | 0.882212733 | 0.938 | 0.728 | 1.61E−38 | 5.48E+37 |
| CD.Plsma/IgM.IGHM.JCHAIN | RPL30 | 7.18E−43 | 0.575758671 | 0.924 | 0.865 | 1.65E−38 | 3.49E+37 |
| CD.Mac.APOE.PTGDS | HLA-DRB5 | 1.17E−42 | 0.890716089 | 0.668 | 0.461 | 2.69E−38 | 3.31E+37 |
| CD.Plsma/IgA.IGLC3.IGLC2.2 | DEFA5 | 1.32E−42 | −1.081309699 | 0.129 | 0.79 | 3.05E−38 | −3.55E+37 |
| CD.T.CD8B.TRGC2 | IGHG3 | 2.60E−42 | −0.876606504 | 0.028 | 0.268 | 5.99E−38 | −1.46E+37 |
| CD.Plsma/IgA.IGHA2.IGKV1-12 | RPS20 | 5.07E−42 | 0.615168863 | 0.951 | 0.867 | 1.17E−37 | 5.27E+36 |
| CD.T.CCR7.GPR183 | DEFA5 | 6.69E−42 | −1.22425943 | 0.135 | 0.372 | 1.54E−37 | −7.95E+36 |
| CD.Plsma/IgA.IGHA2.IGKV1-12 | IGHM | 1.19E−41 | −1.24840858 | 0.934 | 0.828 | 2.73E−37 | −4.57E+36 |
| CD.Plsma/IgA.IGHA2.IGKV1-12 | RPS24 | 1.20E−41 | 0.596720822 | 0.951 | 0.867 | 2.75E−37 | 2.17E+36 |
| CD.Plsma/IgM.IGHM.JCHAIN | MT-CYB | 2.52E−41 | 0.624616588 | 0.966 | 0.967 | 5.81E−37 | 1.08E+36 |
| CD.T.CCR7.SELL | JCHAIN | 4.08E−41 | 0.626592397 | 0.651 | 0.405 | 9.38E−37 | 6.68E+35 |
| CD.Plsma/IgA.IGHA2.IGKV1-12 | RPL30 | 4.78E−41 | 0.592307929 | 0.939 | 0.877 | 1.10E−36 | 5.38E+35 |
| CD.T.CCR7.SELL | RPS10 | 1.55E−40 | 0.510432 | 0.834 | 0.649 | 3.57E−36 | 1.43E+35 |
| CD.NK.CCL3.CD160 | MT-ND1 | 1.58E−40 | 0.604488713 | 0.871 | 0.728 | 3.64E−36 | 1.66E+35 |
| CD.Plsma/IgA.IGHA2.IGKV1-12 | MT-CO1 | 1.80E−40 | 0.479601177 | 0.985 | 0.993 | 4.15E−36 | 1.16E+35 |
| CD.EC.MTRNR2L1.MT-ND3 | IGKC | 2.48E−40 | 0.829069943 | 0.992 | 0.922 | 5.71E−36 | 1.45E+35 |
| CD.Plsma/IgA.IGHA2.IGKV1-12 | MT-CO2 | 5.22E−40 | 0.536787981 | 0.992 | 0.993 | 1.20E−35 | 4.47E+34 |
| CD.Plsma/IgA.IGHA1.IGKC | IGLC2 | 1.11E−39 | 0.570444405 | 0.91 | 0.806 | 2.56E−35 | 2.23E+34 |
| CD.Plsma/IgA.AL9287683.RPL39 | TPT1 | 2.03E−39 | −0.387010115 | 0.991 | 0.992 | 4.68E−35 | −8.28E+33 |
| CD.B.RPS29.RPS21 | RPS10 | 2.23E−39 | 0.495881078 | 0.886 | 0.738 | 5.13E−35 | 9.67E+33 |
| CD.Plsma/IgA.IGHA2.IGKV1-12 | RPS4Y1 | 5.14E−39 | 0.491144315 | 0.576 | 0.143 | 1.18E−34 | 4.15E+33 |
| CD.Plsma/IgM.IGHM.JCHAIN | RPS23 | 5.29E−39 | 0.507346673 | 0.963 | 0.914 | 1.22E−34 | 4.16E+33 |
| CD.NK.CCL3.CD160 | JCHAIN | 6.61E−39 | 0.724652813 | 0.783 | 0.534 | 1.52E−34 | 4.76E+33 |
| CD.Plsma/IgA.IGLL5.IGLV2-14 | EEF1A1 | 7.06E−39 | 0.694168115 | 0.996 | 0.996 | 1.62E−34 | 4.27E+33 |
| CD.Plsma/IgM.IGHM.JCHAIN | RPS20 | 1.12E−38 | 0.479443682 | 0.935 | 0.882 | 2.57E−34 | 1.87E+33 |
| CD.Paneth.DEFA6.ITLN2 | APOA1 | 1.28E−38 | 1.290328567 | 0.532 | 0.004 | 2.95E−34 | 4.37E+33 |
| CD.Plsma/IgA.IGHA2.IGKV1-12 | RPL10 | 1.36E−38 | 0.469754579 | 0.998 | 0.993 | 3.13E−34 | 1.50E+33 |
| CD.Plsma/IgA.IGHA2.IGKV1-12 | RPS29 | 2.80E−38 | −0.5443016 | 0.835 | 0.943 | 6.45E−34 | −8.44E+32 |
| CD.Plsma/IgG.IGHGP.IGHG2 | JSRP1 | 3.19E−38 | 0.41447559 | 0.444 | 0.245 | 7.34E−34 | 5.65E+32 |
| CD.Plsma/IgA.IGHA2.IGKV1-12 | RPS23 | 3.87E−38 | 0.596479617 | 0.968 | 0.938 | 8.91E−34 | 6.70E+32 |
| CD.Plsma/IgM.IGHM.JCHAIN | NACA | 4.28E−38 | 0.579273392 | 0.903 | 0.824 | 9.86E−34 | 5.88E+32 |
| CD.Fibro.TFPI2.CCL13 | TPT1 | 7.06E−38 | 0.942527275 | 1 | 0.978 | 1.62E−33 | 5.80E+32 |
| CD.Plsma/IgA.AL9287683.RPL39 | DEFA6 | 1.36E−37 | −0.479150864 | 0.039 | 0.364 | 3.12E−33 | −1.54E+32 |
| CD.Fibro.TFPI2.CCL13 | EEF1A1 | 2.20E−37 | 0.731661714 | 1 | 0.978 | 5.07E−33 | 1.44E+32 |
| CD.T.CD8B.TRGC2 | IGHG4 | 3.52E−37 | −0.812507356 | 0.023 | 0.231 | 8.10E−33 | −1.00E+32 |
| CD.Plsma/IgM.IGHM.JCHAIN | VIM | 5.85E−37 | 0.703997072 | 0.85 | 0.684 | 1.35E−32 | 5.23E+31 |
| CD.Plsma/IgA.AL9287683.RPL39 | IGHA2 | 6.93E−37 | 1.066804791 | 0.972 | 0.832 | 1.60E−32 | 6.68E+31 |
| CD.T.CCR7.SELL | IGLC2 | 8.23E−37 | 0.642405493 | 0.707 | 0.519 | 1.90E−32 | 3.39E+31 |
| CD.Plsma/IgA.AL9287683.RPL39 | DUSP5 | 1.20E−36 | −0.73400949 | 0.671 | 0.845 | 2.76E−32 | −2.66E+31 |
| CD.Plsma/IgG.IGHGP.IGHG2 | JCHAIN | 2.06E−36 | −0.487708919 | 0.954 | 0.969 | 4.74E−32 | −1.03E+31 |
| CD.Plsma/IgA.AL9287683.RPL39 | RPS4Y1 | 3.06E−36 | −0.481182452 | 0.139 | 0.534 | 7.04E−32 | −6.84E+30 |
| CD.Plsma/IgM.IGHM.JCHAIN | MT-ND3 | 3.20E−36 | 0.486214299 | 0.961 | 0.949 | 7.36E−32 | 6.61E+30 |
| CD.Plsma/IgG.IGHGP.IGHG2 | MT-ND1 | 5.57E−36 | 0.458159711 | 0.903 | 0.851 | 1.28E−31 | 3.57E+30 |
| CD.Plsma/IgM.IGHM.JCHAIN | RPL5 | 5.97E−36 | 0.455203162 | 0.918 | 0.863 | 1.38E−31 | 3.31E+30 |
| CD.Plsma/IgG.IGHGP.IGHG2 | DUSP5 | 6.64E−36 | −0.433204011 | 0.428 | 0.619 | 1.53E−31 | −2.83E+30 |
| CD.Plsma/IgA.IGHA2.IGKV1-12 | RPL13A | 9.60E−36 | 0.44069746 | 0.987 | 0.983 | 2.21E−31 | 1.99E+30 |
| CD.Paneth.DEFA6.ITLN2 | FABP6 | 1.18E−35 | 1.059253389 | 0.512 | 0.008 | 2.73E−31 | 3.88E+30 |
| CD.EC.MTRNR2L1.MT-ND3 | IGHM | 1.56E−35 | 1.090690774 | 0.671 | 0.22 | 3.59E−31 | 3.04E+30 |
| CD.Plsma/IgA.IGHA2.IGKV1-12 | MT-ND4L | 1.96E−35 | −0.602406786 | 0.347 | 0.687 | 4.52E−31 | −1.33E+30 |
| CD.Bclls.CD69.GPR183 | JUN | 2.04E−35 | 0.94084515 | 0.763 | 0.398 | 4.68E−31 | 2.01E+30 |
| CD.Plsma/IgM.IGHM.IGLV2-14 | TPT1 | 2.21E−35 | 0.895946423 | 0.96 | 0.932 | 5.09E−31 | 1.76E+30 |
| CD.Fibro.TFPI2.CCL13 | COL18A1 | 4.63E−35 | −1.601361507 | 0.39 | 0.882 | 1.07E−30 | −1.50E+30 |
| CD.Plsma/IgA.IGHA2.IGKV1-12 | BTG2 | 5.31E−35 | −0.647801018 | 0.716 | 0.882 | 1.22E−30 | −5.30E+29 |
| CD.Plsma/IgM.IGHM.JCHAIN | CD48 | 5.99E−35 | 0.468308981 | 0.522 | 0.163 | 1.38E−30 | 3.40E+29 |
| CD.Bclls.CD69.GPR183 | HSP90AA1 | 7.56E−35 | 1.334329616 | 0.853 | 0.641 | 1.74E−30 | 7.67E+29 |
| CD.Plsma/IgA.IGHA2.IGKV1-12 | RPS12 | 9.33E−35 | 0.492175139 | 0.975 | 0.961 | 2.15E−30 | 2.29E+29 |
| CD.EC.MTRNR2L1.MT-ND3 | MTRNR2L12 | 9.74E−35 | 1.429246273 | 0.775 | 0.416 | 2.24E−30 | 6.38E+29 |
| CD.Plsma/IgA.IGHA2.IGKV1-12 | RPS13 | 1.12E−34 | 0.499765217 | 0.943 | 0.938 | 2.58E−30 | 1.94E+29 |
| CD.Plsma/IgA.IGHA2.IGKV1-12 | RPL13 | 1.65E−34 | 0.428177441 | 0.994 | 0.998 | 3.81E−30 | 1.12E+29 |
| CD.Plsma/IgA.IGHA2.IGKV1-12 | IGKV1-12 | 1.78E−34 | −1.004412128 | 0.816 | 0.874 | 4.11E−30 | −2.45E+29 |
| CD.TA.PLCG2.SOX4 | MT-ND1 | 3.00E−34 | 0.879032375 | 0.994 | 0.975 | 6.90E−30 | 1.27E+29 |
| CD.Plsma/IgA.AL9287683.RPL39 | RPS9 | 3.51E−34 | −0.351352122 | 0.98 | 0.992 | 8.07E−30 | −4.35E+28 |
| CD.EC.MTRNR2L1.MT-ND3 | MT-ATP8 | 3.59E−34 | 1.112089249 | 0.603 | 0.161 | 8.27E−30 | 1.35E+29 |
| CD.B.MT-CO2.MT-CO1 | EIF1AY | 4.89E−34 | 0.817752158 | 0.167 | 0.011 | 1.13E−29 | 7.26E+28 |
| CD.Plsma/IgG.IGHGP.IGHG2 | MT-ATP6 | 5.31E−34 | 0.444472291 | 0.931 | 0.874 | 1.22E−29 | 3.64E+28 |
| CD.NK.CCL3.CD160 | IGHA2 | 7.19E−34 | 0.690971747 | 0.435 | 0.157 | 1.66E−29 | 4.17E+28 |
| CD.T.CCR7.SELL | RPS26 | 7.74E−34 | 0.324272587 | 0.903 | 0.671 | 1.78E−29 | 1.82E+28 |
| CD.Plsma/IgA.AL9287683.RPL39 | RPL19 | 8.72E−34 | −0.354627086 | 0.996 | 0.989 | 2.01E−29 | −1.77E+28 |
| CD.T.CCR7.GPR183 | IGHA1 | 1.24E−33 | 0.414723642 | 0.867 | 0.67 | 2.85E−29 | 1.46E+28 |
| CD.NK.CCL3.CD160 | HSP90AA1 | 2.58E−33 | 0.718509937 | 0.859 | 0.74 | 5.93E−29 | 1.21E+28 |
| CD.T.CCR7.SELL | MT-CO2 | 3.33E−33 | 0.275473386 | 0.986 | 0.954 | 7.67E−29 | 3.59E+27 |
| CD.Fibro.TFPI2.CCL13 | IGKC | 3.56E−33 | 0.760589135 | 1 | 1 | 8.19E−29 | 9.28E+27 |
| CD.Mito.MT-CO2.MT-ND2 | MT-ND1 | 4.30E−33 | 0.647324453 | 0.984 | 0.949 | 9.90E−29 | 6.54E+27 |
| CD.Plsma/IgM.IGHM.JCHAIN | RPL7 | 8.40E−33 | 0.376659823 | 0.973 | 0.982 | 1.93E−28 | 1.95E+27 |
| CD.NK.CCL3.CD160 | XIST | 1.10E−32 | −0.980002196 | 0.063 | 0.262 | 2.53E−28 | −3.88E+27 |
| CD.Plsma/IgM.IGHM.JCHAIN | RPS24 | 1.33E−32 | 0.478827743 | 0.938 | 0.88 | 3.07E−28 | 1.56E+27 |
| CD.Paneth.DEFA6.ITLN2 | IGHA2 | 1.60E−32 | 1.128582109 | 0.751 | 0.157 | 3.68E−28 | 3.07E+27 |
| CD.Fibro.TFPI2.CCL13 | RPL9 | 1.96E−32 | 0.849449568 | 0.99 | 0.946 | 4.51E−28 | 1.89E+27 |
| CD.T.MT-CO2.MT-CO3 | TPT1 | 2.05E−32 | 0.74128659 | 0.936 | 0.873 | 4.72E−28 | 1.57E+27 |
| CD.Plsma/IgM.IGHM.JCHAIN | RPS27A | 2.31E−32 | 0.408977537 | 0.976 | 0.963 | 5.31E−28 | 7.70E+26 |
| CD.Plsma/IgA.HSPA1B.HSPA1A | EEF1A1 | 4.40E−32 | 0.675929935 | 0.988 | 0.993 | 1.01E−27 | 6.68E+26 |
| CD.Plsma/IgA.AL9287683.RPL39 | RPL12 | 5.55E−32 | −0.388826678 | 0.989 | 0.995 | 1.28E−27 | −3.05E+26 |
| CD.Fibro.TFPI2.CCL13 | RPS27A | 7.82E−32 | 0.648071853 | 1 | 0.978 | 1.80E−27 | 3.60E+26 |
| CD.T.KLRB1.LTF | EIF1AY | 1.06E−31 | −0.541457617 | 0.005 | 0.11 | 2.43E−27 | −2.23E+26 |
| CD.Fibro.TFPI2.CCL13 | RPS3A | 1.28E−31 | 0.640922957 | 0.997 | 1 | 2.94E−27 | 2.18E+26 |
| CD.T.CCR7.SELL | IGHM | 1.37E−31 | 0.539053008 | 0.415 | 0.2 | 3.16E−27 | 1.70E+26 |
| CD.Plsma/IgA.IGHA2.IGKV1-12 | RPL28 | 1.68E−31 | 0.49729224 | 0.945 | 0.956 | 3.86E−27 | 1.29E+26 |
| CD.Plsma/IgA.AL9287683.RPL39 | RPL27A | 2.21E−31 | −0.311434323 | 0.993 | 0.993 | 5.10E−27 | −6.11E+25 |
| CD.Plsma/IgA.IGLC3.IGLC2.2 | JCHAIN | 2.72E−31 | 0.50228616 | 1 | 1 | 6.26E−27 | 8.03E+25 |
| CD.Plsma/IgA.IGHA1.IGKC | XIST | 3.75E−31 | −0.481035293 | 0.103 | 0.429 | 8.63E−27 | −5.57E+25 |
| CD.Plsma/IgA.AL9287683.RPL39 | RPL13A | 8.78E−31 | −0.311487744 | 0.996 | 0.998 | 2.02E−26 | −1.54E+25 |
| CD.T.KLRB1.LTF | IGHG3 | 9.04E−31 | −0.689564337 | 0.031 | 0.197 | 2.08E−26 | −3.31E+25 |
| CD.Plsma/IgA.IGHA1.IGKC | MALAT1 | 1.00E−30 | −0.354190295 | 0.974 | 0.988 | 2.31E−26 | −1.53E+25 |
| CD.Fibro.TFPI2.CCL13 | RPS27 | 1.26E−30 | −0.723897097 | 0.984 | 1 | 2.91E−26 | −2.49E+25 |
| CD.Fibro.TFPI2.CCL13 | NACA | 1.36E−30 | 1.030564111 | 0.959 | 0.796 | 3.12E−26 | 3.30E+25 |
| CD.Fibro.TFPI2.CCL13 | RPL7 | 2.26E−30 | 0.643036879 | 0.997 | 0.978 | 5.21E−26 | 1.23E+25 |
| CD.NK.CCL3.CD160 | CMC1 | 2.34E−30 | −0.677153946 | 0.582 | 0.74 | 5.39E−26 | −1.26E+25 |
| CD.Plsma/IgA.AL9287683.RPL39 | RPS26 | 2.36E−30 | 0.435025555 | 0.963 | 0.856 | 5.43E−26 | 8.01E+24 |
| CD.Mono.CXCL10.TNF | RP11-1143G9.4 | 2.48E−30 | −2.065628747 | 0.421 | 0.869 | 5.71E−26 | −3.62E+25 |
| CD.Paneth.DEFA6.ITLN2 | APOA4 | 2.78E−30 | 0.94821013 | 0.424 | 0 | 6.40E−26 | 1.48E+25 |
| CD.T.TRDC.TRGC2 | MT-ND3 | 3.07E−30 | 0.577764063 | 0.961 | 0.811 | 7.07E−26 | 8.18E+24 |
| CD.Plsma/IgA.AL9287683.RPL39 | RPL21 | 3.22E−30 | −0.258438147 | 0.998 | 1 | 7.41E−26 | −3.49E+24 |
| CD.EC.MTRNR2L1.MT-ND3 | IGHA2 | 3.58E−30 | 0.965181553 | 0.514 | 0.113 | 8.24E−26 | 1.17E+25 |
| CD.B.MT-CO2.MT-CO1 | IGHG4 | 4.01E−30 | 0.735445517 | 0.172 | 0.015 | 9.24E−26 | 7.96E+24 |
| CD.T.CCR7.SELL | MT-CO3 | 4.25E−30 | 0.250460152 | 0.991 | 0.95 | 9.78E−26 | 2.56E+24 |
| CD.Plsma/IgA.IGLC3.IGLC2.2 | ZFP36 | 4.75E−30 | 0.980285295 | 0.796 | 0.23 | 1.09E−25 | 8.96E+24 |
| CD.T.CCR7.SELL | DEFA6 | 4.85E−30 | −0.915954309 | 0.027 | 0.176 | 1.12E−25 | −8.21E+24 |
| CD.Plsma/IgA.IGHA1.IGKC | UBC | 5.11E−30 | −0.434378036 | 0.918 | 0.967 | 1.18E−25 | −3.70E+24 |
| CD.Fibro.TFPI2.CCL13 | IGHA2 | 5.19E−30 | 1.402412614 | 0.845 | 0.194 | 1.19E−25 | 1.17E+25 |
| CD.Plsma/IgM.IGHM.JCHAIN | RPL3 | 5.37E−30 | 0.299041754 | 0.984 | 0.982 | 1.24E−25 | 2.42E+24 |
| CD.Plsma/IgA.AL9287683.RPL39 | RPL24 | 7.33E−30 | −0.38649076 | 0.956 | 0.974 | 1.69E−25 | −2.29E+24 |
| CD.Plsma/IgM.IGHM.JCHAIN | EEF1B2 | 8.14E−30 | 0.540888002 | 0.899 | 0.81 | 1.87E−25 | 2.89E+24 |
| CD.T.CD8B.TRGC2 | MT-CYB | 9.05E−30 | 0.395373164 | 0.972 | 0.939 | 2.08E−25 | 1.90E+24 |
| CD.Plsma/IgM.IGHM.JCHAIN | H3F3B | 9.08E−30 | 0.553696313 | 0.948 | 0.912 | 2.09E−25 | 2.65E+24 |
| CD.T.MT-CO2.MT-CO3 | MT-ND1 | 1.21E−29 | 0.755011622 | 0.952 | 0.81 | 2.80E−25 | 2.70E+24 |
| CD.Fibro.TFPI2.CCL13 | JCHAIN | 1.29E−29 | 0.978109869 | 0.987 | 0.731 | 2.97E−25 | 3.29E+24 |
| CD.Plsma/IgA.AL9287683.RPL39 | RPS16 | 1.70E−29 | −0.321840054 | 0.987 | 0.985 | 3.92E−25 | −8.21E+23 |
| CD.Plsma/IgA.IGHA2.IGKV1-12 | MTRNR2L8 | 2.13E−29 | 0.435610538 | 0.424 | 0.086 | 4.91E−25 | 8.88E+23 |
| CD.Plsma/IgA.AL9287683.RPL39 | RPL18 | 2.22E−29 | −0.357077712 | 0.987 | 0.992 | 5.12E−25 | −6.97E+23 |
| CD.Mac.APOE.PTGDS | BRI3 | 2.29E−29 | −0.520918977 | 0.353 | 0.581 | 5.27E−25 | −9.88E+23 |
| CD.Plsma/IgM.IGHM.JCHAIN | RPL10 | 3.07E−29 | 0.377233518 | 0.996 | 0.992 | 7.06E−25 | 5.34E+23 |
| CD.Plsma/IgG.IGHGP.IGHG2 | SPINK4 | 4.31E−29 | 0.288711899 | 0.234 | 0.096 | 9.93E−25 | 2.91E+23 |
| CD.Fibro.TFPI2.CCL13 | TNFRSF11B | 4.33E−29 | −1.732694376 | 0.065 | 0.516 | 9.98E−25 | −1.74E+24 |
| CD.Plsma/IgA.IGHA1.IGKC | FOS | 4.69E−29 | −0.531245793 | 0.714 | 0.923 | 1.08E−24 | −4.92E+23 |
| CD.T.CCR7.SELL | MT-ND4L | 5.77E−29 | −0.912490288 | 0.154 | 0.326 | 1.33E−24 | −6.87E+23 |
| CD.Plsma/IgM.IGHM.JCHAIN | HNRNPA1 | 7.79E−29 | 0.428331603 | 0.823 | 0.614 | 1.79E−24 | 2.39E+23 |
| CD.Plsma/IgA.IGLL5.IGLV2-14 | TPT1 | 8.84E−29 | 0.584795908 | 1 | 0.977 | 2.03E−24 | 2.88E+23 |
| CD.Plsma/IgA.AL9287683.RPL39 | RPS11 | 1.16E−28 | −0.329506014 | 0.978 | 0.979 | 2.68E−24 | −1.23E+23 |
| CD.Plsma/IgA.IGHA2.IGKV1-12 | DEFA5 | 1.33E−28 | 0.337276309 | 0.347 | 0.039 | 3.06E−24 | 1.10E+23 |
| CD.Fibro.TFPI2.CCL13 | RPS24 | 1.49E−28 | 0.696614974 | 0.992 | 0.925 | 3.43E−24 | 2.03E+23 |
| CD.B.MT-CO2.MT-CO1 | AC090498.1 | 1.54E−28 | 0.90423986 | 0.258 | 0.045 | 3.55E−24 | 2.55E+23 |
| CD.Plsma/IgA.IGHA2.IGKV1-12 | MT-ND3 | 1.58E−28 | 0.468483608 | 0.943 | 0.941 | 3.64E−24 | 1.29E+23 |
| CD.Bclls.CD69.GPR183 | EIF1 | 1.58E−28 | 0.542040075 | 0.959 | 0.909 | 3.64E−24 | 1.49E+23 |
| CD.Plsma/IgA.IGLV2-14.IGLV3-19 | EEF1A1 | 1.88E−28 | 1.225719265 | 1 | 0.956 | 4.33E−24 | 2.83E+23 |
| CD.T.KLRB1.LTF | RPS4X | 1.90E−28 | 0.296288781 | 0.995 | 0.983 | 4.37E−24 | 6.79E+22 |
| CD.Plsma/IgA.IGHA2.IGKV1-12 | BTF3 | 1.91E−28 | 0.514715487 | 0.801 | 0.574 | 4.39E−24 | 1.17E+23 |
| CD.Plsma/IgM.IGHM.JCHAIN | EIF1 | 1.99E−28 | 0.341381261 | 0.964 | 0.961 | 4.57E−24 | 7.46E+22 |
| CD.Plsma/IgA.IGLC3.IGLC2.2 | IGHA2 | 2.32E−28 | 1.397707753 | 0.995 | 0.845 | 5.35E−24 | 2.61E+23 |
| CD.Plsma/IgA.IGHA2.IGKV1-12 | IGLC3 | 2.52E−28 | 0.498352831 | 0.864 | 0.68 | 5.81E−24 | 8.58E+22 |
| CD.Plsma/IgA.AL9287683.RPL39 | RPS8 | 2.79E−28 | −0.366761722 | 0.993 | 0.998 | 6.42E−24 | −5.71E+22 |
| CD.EC.MTRNR2L1.MT-ND3 | MT-ND1 | 2.90E−28 | 0.603604402 | 1 | 0.997 | 6.67E−24 | 9.05E+22 |
| CD.Plsma/IgM.IGHM.JCHAIN | RPS14 | 3.25E−28 | 0.313247013 | 0.966 | 0.971 | 7.48E−24 | 4.19E+22 |
| CD.Plsma/IgA.IGKV1-12.IGHA2 | TPT1 | 3.72E−28 | 1.27607481 | 0.994 | 0.966 | 8.56E−24 | 1.49E+23 |
| CD.Mono.CXCL10.TNF | HSPA1B | 4.15E−28 | −1.784316443 | 0.037 | 0.621 | 9.55E−24 | −1.87E+23 |
| CD.Plsma/IgA.IGLL5.IGLV2-14 | RPL9 | 5.05E−28 | 0.5308694 | 1 | 0.932 | 1.16E−23 | 4.57E+22 |
| CD.T.CCL4.GMZK | MT-ND1 | 5.38E−28 | 0.517145451 | 0.81 | 0.562 | 1.24E−23 | 4.17E+22 |
| CD.Plsma/IgA.AL9287683.RPL39 | RPL13 | 7.39E−28 | −0.257680644 | 0.993 | 0.997 | 1.70E−23 | −1.51E+22 |
| CD.Plsma/IgA.AL9287683.RPL39 | RPLP2 | 9.56E−28 | −0.390021256 | 0.993 | 0.993 | 2.20E−23 | −1.77E+22 |
| CD.Prolif/EC.UBE2C.CCNB2 | IGHA1 | 9.88E−28 | 1.490051011 | 1 | 0.924 | 2.27E−23 | 6.55E+22 |
| CD.Fibro.TFPI2.CCL13 | RPS29 | 1.08E−27 | −1.062845632 | 0.786 | 0.914 | 2.48E−23 | −4.28E+22 |
| CD.Plsma/IgA.IGLC3.IGLC2.2 | HSPA1B | 1.10E−27 | 1.171896922 | 0.683 | 0.155 | 2.53E−23 | 4.63E+22 |
| CD.Plsma/IgA.AL9287683.RPL39 | MZB1 | 1.36E−27 | −0.376458855 | 0.965 | 0.982 | 3.14E−23 | −1.20E+22 |
| CD.Mono.CXCL10.TNF | HLA-DRB5 | 1.38E−27 | −1.113922125 | 0.085 | 0.732 | 3.17E−23 | −3.51E+22 |
| CD.Plsma/IgA.IGHA2.IGKV1-12 | RPL21 | 1.51E−27 | 0.368888379 | 0.987 | 0.985 | 3.48E−23 | 1.06E+22 |
| CD.Plsma/IgA.IGHA1.IGKC | IGHV3-64 | 2.44E−27 | 0.338772891 | 0.167 | 0.021 | 5.61E−23 | 6.04E+21 |
| CD.Plsma/IgA.IGKV1-12.IGHA2 | RPL9 | 2.78E−27 | 1.110783613 | 0.986 | 0.915 | 6.40E−23 | 1.74E+22 |
| CD.Plsma/IgA.IGHA2.IGKV1-12 | RPS17 | 2.79E−27 | 0.427381649 | 0.964 | 0.948 | 6.42E−23 | 6.66E+21 |
| CD.T.CCR7.GPR183 | REG1A | 2.88E−27 | −0.888392834 | 0.094 | 0.272 | 6.62E−23 | −1.34E+22 |
| CD.Mito.MT-CO2.MT-ND2 | HSPA1A | 3.61E−27 | 1.108454945 | 0.623 | 0.351 | 8.32E−23 | 1.33E+22 |
| CD.NK.CCL3.CD160 | HSPE1 | 3.87E−27 | 0.678673883 | 0.632 | 0.423 | 8.90E−23 | 7.62E+21 |
| CD.Plsma/IgA.AL9287683.RPL39 | FAU | 4.43E−27 | −0.328252117 | 0.987 | 0.987 | 1.02E−22 | −3.22E+21 |
| CD.Plsma/IgA.IGLL5.IGLV2-14 | MT-CO3 | 5.29E−27 | 0.588390056 | 0.975 | 0.955 | 1.22E−22 | 4.83E+21 |
| CD.T.CCR7.GPR183 | IGHM | 5.77E−27 | 0.471828099 | 0.413 | 0.206 | 1.33E−22 | 3.56E+21 |
| CD.Bclls.CD69.GPR183 | HSPA1A | 5.77E−27 | 1.126958161 | 0.526 | 0.217 | 1.33E−22 | 8.48E+21 |
| CD.Fibro.TFPI2.CCL13 | COL1A2 | 6.05E−27 | −0.90539105 | 0.853 | 0.978 | 1.39E−22 | −6.50E+21 |
| CD.Plsma/IgA.AL9287683.RPL39 | RPS13 | 6.86E−27 | −0.270754087 | 0.983 | 0.995 | 1.58E−22 | −1.72E+21 |
| CD.Plsma/IgA.IGLL5.IGLV2-14 | MT-ND1 | 8.05E−27 | 0.632524963 | 0.979 | 0.905 | 1.85E−22 | 3.41E+21 |
| CD.Plsma/IgM.IGHM.JCHAIN | RPL21 | 8.23E−27 | 0.31740833 | 0.984 | 0.98 | 1.89E−22 | 1.68E+21 |
| CD.Mac.APOE.PTGDS | PTPRC | 8.35E−27 | −0.49336668 | 0.473 | 0.7 | 1.92E−22 | −2.57E+21 |
| CD.T.CCL4.GMZK | RPS29 | 8.71E−27 | 0.354827566 | 0.955 | 0.945 | 2.00E−22 | 1.77E+21 |
| CD.Plsma/IgG.IGHGP.IGHG2 | H3F3B | 8.80E−27 | 0.392770323 | 0.91 | 0.906 | 2.03E−22 | 1.94E+21 |
| CD.Plsma/IgG.IGHGP.IGHG2 | PRDX4 | 8.97E−27 | 0.287602569 | 0.836 | 0.826 | 2.06E−22 | 1.39E+21 |
| CD.B.IGHG1.IGLC2 | HLA-DQA2 | 9.57E−27 | −1.024436118 | 0.004 | 0.168 | 2.20E−22 | −4.65E+21 |
| CD.Fibro.TFPI2.CCL13 | IGHG4 | 9.89E−27 | −0.811627465 | 0.021 | 0.376 | 2.28E−22 | −3.57E+21 |
| CD.Mono.CXCL10.TNF | IGKC | 9.93E−27 | 1.236112476 | 1 | 0.987 | 2.29E−22 | 5.41E+21 |
| CD.Plsma/IgA.IGKV1-12.IGHA2 | IGHA2 | 1.01E−26 | −1.03638696 | 1 | 1 | 2.32E−22 | −4.47E+21 |
| CD.B.MT-CO2.MT-CO1 | B2M | 1.06E−26 | −0.316031758 | 0.995 | 0.999 | 2.44E−22 | −1.29E+21 |
| CD.Plsma/IgG.IGHGP.IGHG2 | IGHG4 | 1.14E−26 | −0.306774429 | 0.909 | 0.921 | 2.62E−22 | −1.17E+21 |
| CD.Paneth.DEFA6.ITLN2 | MIF | 1.21E−26 | 0.835744974 | 0.634 | 0.157 | 2.79E−22 | 3.00E+21 |
| CD.Fibro.TFPI2.CCL13 | RPL36 | 1.32E−26 | −0.698361158 | 0.902 | 0.978 | 3.04E−22 | −2.30E+21 |
| CD.NK.CCL3.CD160 | HSPA1A | 1.93E−26 | 0.7691821 | 0.412 | 0.181 | 4.44E−22 | 1.73E+21 |
| CD.NK.CCL3.CD160 | NKG7 | 2.41E−26 | −0.32745046 | 0.919 | 0.946 | 5.55E−22 | −5.90E+20 |
| CD.Plsma/IgA.IGHA2.IGKV1-12 | PRDX4 | 2.72E−26 | 0.426413071 | 0.922 | 0.833 | 6.27E−22 | 6.80E+20 |
| CD.Plsma/IgM.IGHM.JCHAIN | GNB2L1 | 2.88E−26 | 0.347237032 | 0.938 | 0.873 | 6.62E−22 | 5.24E+20 |
| CD.Plsma/IgA.IGLV2-14.IGLV3-19 | MT-ND1 | 2.92E−26 | 1.185981622 | 0.976 | 0.824 | 6.72E−22 | 1.76E+21 |
| CD.Plsma/IgA.IGHA2.IGKV1-12 | GNB2L1 | 2.97E−26 | 0.451171886 | 0.928 | 0.894 | 6.84E−22 | 6.59E+20 |
| CD.Fibro.TFPI2.CCL13 | MT-ND1 | 3.22E−26 | 0.852448011 | 0.995 | 0.914 | 7.40E−22 | 1.15E+21 |
| CD.Plsma/IgG.IGHGP.IGHG2 | IGLC2 | 3.74E−26 | 0.369063037 | 0.935 | 0.89 | 8.61E−22 | 4.29E+20 |
| CD.Plsma/IgM.IGHM.JCHAIN | HSPE1 | 4.24E−26 | 0.596906973 | 0.747 | 0.478 | 9.77E−22 | 6.11E+20 |
| CD.Plsma/IgG.IGHGP.IGHG2 | MT-CO3 | 4.69E−26 | 0.335574032 | 0.968 | 0.96 | 1.08E−21 | 3.11E+20 |
| CD.Mac.APOE.PTGDS | CD52 | 4.80E−26 | −0.614487533 | 0.167 | 0.401 | 1.10E−21 | −5.57E+20 |
| CD.Plsma/IgA.AL9287683.RPL39 | RPS19 | 5.08E−26 | −0.291089893 | 0.993 | 1 | 1.17E−21 | −2.49E+20 |
| CD.B.MT-ND4L.IGHG1 | DEFA5 | 5.26E−26 | 0.768021311 | 0.119 | 0.005 | 1.21E−21 | 6.35E+20 |
| CD.Plsma/IgA.AL9287683.RPL39 | RPSA | 5.57E−26 | −0.328794012 | 0.932 | 0.956 | 1.28E−21 | −2.56E+20 |
| CD.Plsma/IgA.IGHA1.IGKC | MT-ND4L | 8.15E−26 | −0.419313464 | 0.222 | 0.513 | 1.88E−21 | −2.23E+20 |
| CD.Plsma/IgA.IGHA2.IGKV1-12 | CYBA | 8.61E−26 | 0.357129695 | 0.97 | 0.978 | 1.98E−21 | 1.80E+20 |
| CD.B.MT-CO2.MT-CO1 | IGHA2 | 1.06E−25 | 0.879738775 | 0.306 | 0.071 | 2.45E−21 | 3.59E+20 |
| CD.Plsma/IgA.AL9287683.RPL39 | RPL7A | 1.13E−25 | −0.333564577 | 0.98 | 0.982 | 2.59E−21 | −1.29E+20 |
| CD.Plsma/IgA.IGKV1-12.IGHA2 | MT-ND4L | 1.31E−25 | −1.087416971 | 0.359 | 0.932 | 3.03E−21 | −3.59E+20 |
| CD.Plsma/IgM.IGHM.IGLV2-14 | RPL9 | 1.39E−25 | 0.677267591 | 0.9 | 0.88 | 3.20E−21 | 2.12E+20 |
| CD.Plsma/IgA.AL9287683.RPL39 | MT1G | 1.49E−25 | −0.512751257 | 0.009 | 0.23 | 3.42E−21 | −1.50E+20 |
| CD.T.CCL4.GMZK | MT-CO3 | 1.74E−25 | 0.316932102 | 0.982 | 0.914 | 4.01E−21 | 7.90E+19 |
| CD.B.MT-CO2.MT-CO1 | RPS26 | 2.19E−25 | 1.055358675 | 0.675 | 0.385 | 5.03E−21 | 2.10E+20 |
| CD.Fibro.TFPI2.CCL13 | EIF5A | 2.33E−25 | −0.840183622 | 0.114 | 0.613 | 5.36E−21 | −1.57E+20 |
| CD.Plsma/IgA.IGHA2.IGKV1-12 | HSPE1 | 2.52E−25 | 0.633723099 | 0.72 | 0.424 | 5.79E−21 | 1.09E+20 |
| CD.Bclls.CD69.GPR183 | RPS26 | 2.52E−25 | −0.522052598 | 0.748 | 0.941 | 5.80E−21 | −9.00E+19 |
| CD.Plsma/IgA.IGHA2.IGKV1-12 | RPL14 | 2.71E−25 | 0.425664008 | 0.93 | 0.897 | 6.24E−21 | 6.82E+19 |
| CD.Plsma/IgA.IGKV1-12.IGHA2 | MT-ND1 | 2.86E−25 | 1.383908013 | 0.964 | 0.881 | 6.58E−21 | 2.10E+20 |
| CD.Plsma/IgA.IGKV1-12.IGHA2 | IGHG1 | 3.44E−25 | −1.028439234 | 0.384 | 0.949 | 7.92E−21 | −1.30E+20 |
| CD.T.CD8B.TRGC2 | RPS10 | 3.46E−25 | 0.58864775 | 0.755 | 0.576 | 7.98E−21 | 7.38E+19 |
| CD.Plsma/IgM.IGHM.JCHAIN | SEC61B | 3.48E−25 | 0.383270569 | 0.93 | 0.871 | 8.02E−21 | 4.78E+19 |
| CD.Mac.APOE.PTGDS | APOE | 3.63E−25 | −0.42814411 | 0.724 | 0.911 | 8.35E−21 | −5.12E+19 |
| CD.Plsma/IgG.IGHGP.IGHG2 | IGHV6-1 | 3.85E−25 | −0.257471634 | 0.018 | 0.109 | 8.86E−21 | −2.90E+19 |
| CD.Plsma/IgM.IGHM.IGLV2-14 | RPS20 | 4.21E−25 | 0.704237946 | 0.889 | 0.816 | 9.68E−21 | 7.27E+19 |
| CD.Plsma/IgA.IGHA1.IGKC | PRDX4 | 4.70E−25 | 0.38878238 | 0.91 | 0.815 | 1.08E−20 | 3.59E+19 |
| CD.TA.PLCG2.SOX4 | MT-ATP6 | 5.03E−25 | 0.605585456 | 0.994 | 0.992 | 1.16E−20 | 5.23E+19 |
| CD.Plsma/IgA.AL9287683.RPL39 | RPLPO | 5.27E−25 | −0.320001501 | 0.978 | 0.99 | 1.21E−20 | −2.64E+19 |
| CD.Plsma/IgA.AL9287683.RPL39 | RPS23 | 5.67E−25 | −0.322128029 | 0.987 | 0.992 | 1.30E−20 | −2.47E+19 |
| CD.NK.CCL3.CD160 | RPL30 | 6.53E−25 | 0.365461414 | 0.901 | 0.752 | 1.50E−20 | 2.43E+19 |
| CD.T.CCR7.SELL | MT-ND1 | 6.96E−25 | 0.348010358 | 0.815 | 0.643 | 1.60E−20 | 2.17E+19 |
| CD.Plsma/IgM.IGHM.JCHAIN | BTF3 | 7.19E−25 | 0.430838029 | 0.811 | 0.618 | 1.66E−20 | 2.60E+19 |
| CD.Plsma/IgM.IGHM.JCHAIN | CIRBP | 7.62E−25 | 0.458675247 | 0.871 | 0.72 | 1.75E−20 | 2.62E+19 |
| CD.Fibro.TFPI2.CCL13 | RPL10 | 8.47E−25 | 0.478861003 | 0.997 | 0.968 | 1.95E−20 | 2.46E+19 |
| CD.Plsma/IgA.AL9287683.RPL39 | RPS2 | 8.53E−25 | −0.346751723 | 0.996 | 0.997 | 1.96E−20 | −1.77E+19 |
| CD.Plsma/IgM.IGHM.IGLV2-14 | RPL30 | 8.78E−25 | 0.6225332 | 0.908 | 0.808 | 2.02E−20 | 3.08E+19 |
| CD.Plsma/IgA.IGKV1-12.IGHA2 | IGKV1-12 | 1.16E−24 | −0.688018089 | 1 | 1 | 2.67E−20 | −2.58E+19 |
| CD.Plsma/IgA.IGLL5.IGLV2-14 | CD48 | 1.22E−24 | 0.686142447 | 0.54 | 0.117 | 2.80E−20 | 2.45E+19 |
| CD.cDC1.CLEC9A.XCR1 | HSPA1A | 1.74E−24 | 1.278717011 | 0.919 | 0.788 | 4.02E−20 | 3.18E+19 |
| CD.Plsma/IgM.IGHM.JCHAIN | RPS13 | 1.78E−24 | 0.402860477 | 0.949 | 0.924 | 4.10E−20 | 9.84E+18 |
| CD.Mstcl.CTSG.CD69 | RPS27 | 2.35E−24 | 0.551756387 | 0.997 | 0.809 | 5.40E−20 | 1.02E+19 |
| CD.Mstcl.CTSG.CD69 | MALAT1 | 2.38E−24 | 0.42062844 | 1 | 0.996 | 5.48E−20 | 7.67E+18 |
| CD.Plsma/IgA.IGHA2.IGKV1-12 | RPL41 | 2.46E−24 | −0.278835948 | 0.966 | 0.998 | 5.66E−20 | −4.93E+18 |
| CD.Plsma/IgA.IGKV1-12.IGHA2 | RPS3A | 2.71E−24 | 0.894946107 | 0.997 | 0.983 | 6.23E−20 | 1.44E+19 |
| CD.Plsma/IgA.AL9287683.RPL39 | RPL26 | 2.75E−24 | −0.294706305 | 0.98 | 0.99 | 6.34E−20 | −4.65E+18 |
| CD.Plsma/IgG.IGHGP.IGHG2 | SAT1 | 3.08E−24 | 0.43632749 | 0.817 | 0.759 | 7.08E−20 | 6.16E+18 |
| CD.Bclls.CD69.GPR183 | TMSB4X | 3.44E−24 | −0.345237554 | 1 | 0.999 | 7.92E−20 | −4.36E+18 |
| CD.Plsma/IgA.IGLV2-14.IGLV3-19 | TPT1 | 3.59E−24 | 1.213921406 | 0.994 | 0.985 | 8.27E−20 | 1.47E+19 |
| CD.NK.CCL3.CD160 | TMSB10 | 3.96E−24 | −0.30427132 | 0.91 | 0.953 | 9.12E−20 | −3.33E+18 |
| CD.Plsma/IgM.IGHM.JCHAIN | RPS3 | 4.58E−24 | 0.286441773 | 0.954 | 0.943 | 1.05E−19 | 2.72E+18 |
| CD.Plsma/IgM.IGHM.JCHAIN | HSPA1A | 4.61E−24 | 0.82366857 | 0.819 | 0.657 | 1.06E−19 | 7.76E+18 |
| CD.Plsma/IgA.IGKV1-12.IGHA2 | EEF1A1 | 4.90E−24 | 0.907287537 | 0.997 | 0.966 | 1.13E−19 | 8.05E+18 |
| CD.Fibro.TFPI2.CCL13 | TFPI2 | 5.03E−24 | −1.680266588 | 0.419 | 0.817 | 1.16E−19 | −1.45E+19 |
| CD.T.CCL4.GMZK | MT-ND3 | 5.78E−24 | 0.433718405 | 0.911 | 0.778 | 1.33E−19 | 3.26E+18 |
| CD.Plsma/IgA.IGHA1.IGKC | HSPA1B | 7.27E−24 | −0.60082649 | 0.175 | 0.463 | 1.67E−19 | −3.59E+18 |
| CD.Plsma/IgA.IGLV2-14.IGLV3-19 | RPS3A | 7.73E−24 | 0.961421719 | 0.994 | 0.971 | 1.78E−19 | 5.40E+18 |
| CD.Plsma/IgM.IGHM.JCHAIN | RPL13A | 8.31E−24 | 0.29461381 | 0.978 | 0.99 | 1.91E−19 | 1.54E+18 |
| CD.Plsma/IgM.IGHM.JCHAIN | RPL11 | 8.62E−24 | 0.299558228 | 0.967 | 0.965 | 1.98E−19 | 1.51E+18 |
| CD.Bclls.CD69.GPR183 | DNAJA1 | 8.90E−24 | 0.834664494 | 0.737 | 0.521 | 2.05E−19 | 4.07E+18 |
| CD.Plsma/IgA.IGHA1.IGKC | FKBP11 | 9.18E−24 | −0.327328989 | 0.934 | 0.966 | 2.11E−19 | −1.55E+18 |
| CD.Plsma/IgM.IGHM.JCHAIN | RPS16 | 9.73E−24 | 0.322480814 | 0.951 | 0.929 | 2.24E−19 | 1.44E+18 |
| CD.Mito.MT-CO2.MT-ND2 | MT-ND3 | 9.94E−24 | 0.460773544 | 0.992 | 0.969 | 2.29E−19 | 2.01E+18 |
| CD.Fibro.TFPI2.CCL13 | IGHA1 | 9.96E−24 | 0.604786437 | 0.997 | 0.989 | 2.29E−19 | 2.64E+18 |
| CD.Bclls.CD69.GPR183 | TPT1 | 1.01E−23 | 0.649745656 | 0.947 | 0.868 | 2.32E−19 | 2.80E+18 |
| CD.Mac.APOE.PTGDS | CTSZ | 1.06E−23 | −0.524056799 | 0.509 | 0.712 | 2.44E−19 | −2.15E+18 |
| CD.Plsma/IgA.IGHA2.IGKV1-12 | RPS16 | 1.09E−23 | 0.390215876 | 0.96 | 0.943 | 2.51E−19 | 1.55E+18 |
| CD.Paneth.DEFA6.ITLN2 | APOC3 | 1.20E−23 | 0.676536993 | 0.351 | 0.004 | 2.77E−19 | 2.44E+18 |
| CD.Plsma/IgA.IGHA2.IGKV1-12 | CFD | 1.28E−23 | 0.263887749 | 0.242 | 0.01 | 2.95E−19 | 8.94E+17 |
| CD.Goblet.FCGBP.SPINK4 | MT-ND1 | 1.32E−23 | 0.841318322 | 1 | 1 | 3.04E−19 | 2.76E+18 |
| CD.Mac.APOE.PTGDS | MT-ND5 | 1.34E−23 | −0.401192169 | 0.593 | 0.773 | 3.09E−19 | −1.30E+18 |
| CD.Plsma/IgA.IGHA2.IGKV1-12 | RPS3 | 1.47E−23 | 0.363120924 | 0.962 | 0.956 | 3.39E−19 | 1.07E+18 |
| CD.NK.CCL3.CD160 | AREG | 1.62E−23 | −0.638267424 | 0.559 | 0.701 | 3.72E−19 | −1.71E+18 |
| CD.Plsma/IgA.IGHA1.IGKC | RPS4Y1 | 1.76E−23 | 0.251717113 | 0.291 | 0.1 | 4.06E−19 | 6.20E+17 |
| CD.B.MT-CO2.MT-CO1 | RPL39 | 1.83E−23 | 0.433869537 | 0.952 | 0.968 | 4.22E−19 | 1.03E+18 |
| CD.Plsma/IgA.IGKV1-12.IGHA2 | MT-ND2 | 2.01E−23 | 1.085961641 | 0.98 | 1 | 4.62E−19 | 2.35E+18 |
| CD.Plsma/IgA.AL9287683.RPL39 | RPS15 | 2.19E−23 | −0.261995427 | 0.996 | 0.993 | 5.05E−19 | −5.19E+17 |
| CD.Fibro.TFPI2.CCL13 | RPL41 | 2.36E−23 | −0.582736828 | 0.992 | 0.968 | 5.43E−19 | −1.07E+18 |
| CD.Plsma/IgA.IGLC3.IGLC2.2 | XIST | 2.38E−23 | 0.484157652 | 0.495 | 0.035 | 5.49E−19 | 8.82E+17 |
| CD.Plsma/IgA.IGHA1.IGLV6-57 | EEF1A1 | 2.43E−23 | 0.906308946 | 1 | 0.969 | 5.60E−19 | 1.62E+18 |
| CD.T.CD8B.TRGC2 | IGHG1 | 2.48E−23 | −0.616829104 | 0.033 | 0.193 | 5.70E−19 | −1.08E+18 |
| CD.T.CARD16.GB2 | RPL41 | 2.60E−23 | 0.30114559 | 0.984 | 0.99 | 5.98E−19 | 5.04E+17 |
| CD.Mono.S100A8.S100A9 | RP11-1143G9.4 | 2.70E−23 | −1.739079433 | 0.581 | 0.845 | 6.21E−19 | −2.80E+18 |
| CD.Plsma/IgA.AL9287683.RPL39 | EIF1AY | 2.77E−23 | −0.337796784 | 0.096 | 0.366 | 6.37E−19 | −5.31E+17 |
| CD.Plsma/IgA.IGHA1.IGKC | IGHM | 2.99E−23 | −0.942670394 | 0.553 | 0.837 | 6.87E−19 | −1.37E+18 |
| CD.Fibro.TFPI2.CCL13 | RPL30 | 4.08E−23 | 0.701053614 | 0.979 | 0.903 | 9.39E−19 | 7.46E+17 |
| CD.Plsma/IgA.AL9287683.RPL39 | HSP90B1 | 4.21E−23 | −0.399100602 | 0.952 | 0.956 | 9.69E−19 | −4.12E+17 |
| CD.B.RPS29.RPS21 | EIF1AY | 4.57E−23 | −0.483463487 | 0.046 | 0.209 | 1.05E−18 | −4.60E+17 |
| CD.Plsma/IgM.IGHM.JCHAIN | RPS12 | 4.74E−23 | 0.348680777 | 0.952 | 0.955 | 1.09E−18 | 3.20E+17 |
| CD.T.CARD16.GB2 | MT-CO3 | 4.74E−23 | 0.4366879 | 0.958 | 0.819 | 1.09E−18 | 4.00E+17 |
| CD.Plsma/IgA.IGHA1.IGKC | HSP90AA1 | 4.80E−23 | −0.604439106 | 0.651 | 0.828 | 1.11E−18 | −5.47E+17 |
| CD.T.MT-CO2.MT-CO3 | MT-ND3 | 4.84E−23 | 0.493280751 | 0.978 | 0.895 | 1.11E−18 | 4.43E+17 |
| CD.NK.CCL3.CD160 | KLRD1 | 5.16E−23 | −0.480234163 | 0.684 | 0.793 | 1.19E−18 | −4.04E+17 |
| CD.cDC1.CLEC9A.XCR1 | HSP90AA1 | 5.60E−23 | 0.918714182 | 0.994 | 0.985 | 1.29E−18 | 7.12E+17 |
| CD.Bclls.CD69.GPR183 | SRSF3 | 6.21E−23 | 0.765689794 | 0.658 | 0.374 | 1.43E−18 | 5.35E+17 |
| CD.Plsma/IgA.IGHA2.IGKV1-12 | TMEM59 | 6.28E−23 | 0.37864514 | 0.928 | 0.86 | 1.45E−18 | 2.62E+17 |
| CD.Plsma/IgA.IGLC3.IGLC2.2 | HLA-B | 6.39E−23 | −0.559160959 | 0.989 | 1 | 1.47E−18 | −3.80E+17 |
| CD.Plsma/IgA.AL9287683.RPL39 | RPL28 | 6.47E−23 | −0.283418667 | 0.989 | 0.989 | 1.49E−18 | −1.90E+17 |
| CD.Plsma/IgA.IGHA2.IGKV1-12 | RPL26 | 7.20E−23 | 0.397931231 | 0.939 | 0.921 | 1.66E−18 | 2.40E+17 |
| CD.Plsma/IgA.AL9287683.RPL39 | RPS25 | 7.39E−23 | −0.280448515 | 0.993 | 0.989 | 1.70E−18 | −1.65E+17 |
| CD.Bclls.CD69.GPR183 | NFKBIA | 7.48E−23 | 1.001570566 | 0.553 | 0.266 | 1.72E−18 | 5.82E+17 |
| CD.T.CD8B.TRGC2 | XIST | 7.67E−23 | 0.828066559 | 0.244 | 0.021 | 1.77E−18 | 4.69E+17 |
| CD.B.MT-CO2.MT-CO1 | MALAT1 | 8.17E−23 | 0.405495588 | 0.995 | 0.998 | 1.88E−18 | 2.16E+17 |
| CD.Plsma/IgA.IGHA1.IGKC | DUSP5 | 9.61E−23 | −0.428422185 | 0.344 | 0.601 | 2.21E−18 | −1.94E+17 |
| CD.Paneth.DEFA6.ITLN2 | AC090498.1 | 9.68E−23 | 0.697248735 | 0.439 | 0.051 | 2.23E−18 | 3.13E+17 |
| CD.Fibro.TFPI2.CCL13 | RPS23 | 1.13E−22 | 0.596459839 | 0.992 | 0.946 | 2.60E−18 | 2.30E+17 |
| CD.Plsma/IgA.AL9287683.RPL39 | DERL3 | 1.24E−22 | −0.401683432 | 0.902 | 0.925 | 2.85E−18 | −1.41E+17 |
| CD.Plsma/IgA.IGLC3.IGLC2.2 | B2M | 1.30E−22 | −0.43309672 | 0.995 | 1 | 2.98E−18 | −1.45E+17 |
| CD.Mono.CXCL10.TNF | HLA-DQA2 | 1.30E−22 | −1.205076303 | 0.043 | 0.562 | 3.00E−18 | −4.01E+17 |
| CD.Plsma/IgM.IGHM.JCHAIN | RPL13 | 1.38E−22 | 0.286896126 | 0.985 | 0.994 | 3.17E−18 | 9.06E+16 |
| CD.Plsma/IgA.IGLV2-14.IGLV3-19 | RPL9 | 1.55E−22 | 1.004368329 | 0.994 | 0.956 | 3.57E−18 | 2.81E+17 |
| CD.Plsma/IgA.IGLC3.IGLC2.1 | IGHG1 | 1.55E−22 | 0.707228599 | 0.687 | 0.392 | 3.58E−18 | 1.98E+17 |
| CD.B.IGHG1.IGLC2 | RPL41 | 1.70E−22 | 0.401269867 | 0.999 | 1 | 3.92E−18 | 1.02E+17 |
| CD.T.CCL4.GMZK | PABPC1 | 1.72E−22 | 0.507573344 | 0.761 | 0.558 | 3.96E−18 | 1.28E+17 |
| CD.Plsma/IgA.IGHA2.IGKV1-12 | HINT1 | 1.85E−22 | 0.4403715 | 0.841 | 0.697 | 4.26E−18 | 1.03E+17 |
| CD.Plsma/IgA.IGHA2.IGKV1-12 | RPL18 | 2.51E−22 | 0.388806298 | 0.968 | 0.951 | 5.78E−18 | 6.72E+16 |
| CD.T.CD8B.TRGC2 | MT-CO3 | 3.28E−22 | 0.290117995 | 0.993 | 0.984 | 7.54E−18 | 3.85E+16 |
| CD.Paneth.DEFA6.ITLN2 | MT-ATP8 | 3.37E−22 | 0.724033441 | 0.463 | 0.063 | 7.76E−18 | 9.33E+16 |
| CD.Plsma/IgA.IGKV1-12.IGHA2 | NACA | 3.91E−22 | 0.951481344 | 0.955 | 0.831 | 9.00E−18 | 1.06E+17 |
| CD.Mito.MT-CO2.MT-ND2 | MTRNR2L12 | 3.97E−22 | 1.133482371 | 0.288 | 0.071 | 9.15E−18 | 1.24E+17 |
| CD.T.CCR7.GPR183 | JCHAIN | 4.01E−22 | 0.362878786 | 0.632 | 0.416 | 9.24E−18 | 3.93E+16 |
| CD.Mono.CXCL10.TNF | IGLC2 | 4.26E−22 | 1.279657409 | 0.933 | 0.719 | 9.80E−18 | 1.31E+17 |
| CD.Plsma/IgM.IGHM.JCHAIN | MT-CO1 | 4.80E−22 | 0.372318898 | 0.972 | 0.992 | 1.11E−17 | 3.37E+16 |
| CD.T.CARD16.GB2 | PTPRC | 5.59E−22 | −0.587137384 | 0.71 | 0.852 | 1.29E−17 | −4.57E+16 |
| CD.NK.CCL3.CD160 | H3F3B | 7.28E−22 | 0.303450824 | 0.967 | 0.929 | 1.68E−17 | 1.81E+16 |
| CD.T.CD8B.TRGC2 | EIF1AY | 7.64E−22 | −0.496049254 | 0.006 | 0.115 | 1.76E−17 | −2.82E+16 |
| CD.B.MT-CO2.MT-CO1 | RPL18A | 7.94E−22 | −0.371154039 | 0.952 | 0.986 | 1.83E−17 | −2.03E+16 |
| CD.B.MT-CO2.MT-CO1 | RPS29 | 8.19E−22 | 0.49906297 | 0.914 | 0.891 | 1.89E−17 | 2.65E+16 |
| CD.Mac.APOE.PTGDS | IFI30 | 8.42E−22 | −0.460310871 | 0.306 | 0.52 | 1.94E−17 | −2.37E+16 |
| CD.cDC1.CLEC9A.XCR1 | AC090498.1 | 8.58E−22 | 0.769816688 | 0.798 | 0.333 | 1.97E−17 | 3.90E+16 |
| CD.Plsma/IgM.IGHM.JCHAIN | RPL22 | 9.00E−22 | 0.405459174 | 0.874 | 0.765 | 2.07E−17 | 1.96E+16 |
| CD.Prolif/EC.UBE2C.CCNB2 | IGKC | 9.07E−22 | 1.273468591 | 0.98 | 0.984 | 2.09E−17 | 6.10E+16 |
| CD.B.MT-CO2.MT-CO1 | RPS27 | 9.29E−22 | 0.332195506 | 0.976 | 0.993 | 2.14E−17 | 1.55E+16 |
| CD.T.CCL20.RORA | RPS27 | 9.29E−22 | 0.32237867 | 0.989 | 0.995 | 2.14E−17 | 1.51E+16 |
| CD.NK.GNLY.KLRC1 | IGKC | 9.92E−22 | 0.909954159 | 0.993 | 0.97 | 2.28E−17 | 3.99E+16 |
| CD.Mac.APOE.PTGDS | MALAT1 | 1.17E−21 | −0.373698203 | 0.795 | 0.893 | 2.70E−17 | −1.38E+16 |
| CD.Fibro.TFPI2.CCL13 | RPS28 | 1.18E−21 | −0.545135424 | 0.977 | 0.968 | 2.71E−17 | −2.01E+16 |
| CD.Plsma/IgA.AL9287683.RPL39 | RPS17 | 1.32E−21 | −0.251400131 | 0.987 | 0.985 | 3.04E−17 | −8.27E+15 |
| CD.Plsma/IgA.AL9287683.RPL39 | RPS20 | 1.38E−21 | −0.307458696 | 0.972 | 0.977 | 3.17E−17 | −9.69E+15 |
| CD.Plsma/IgM.IGHM.JCHAIN | RPL28 | 1.46E−21 | 0.356323983 | 0.945 | 0.906 | 3.37E−17 | 1.06E+16 |
| CD.T.MT-CO2.MT-CO3 | IGKC | 1.49E−21 | 0.665143845 | 0.971 | 0.876 | 3.44E−17 | 1.93E+16 |
| CD.Fibro.TFPI2.CCL13 | RPS21 | 1.60E−21 | −0.975225627 | 0.452 | 0.849 | 3.69E−17 | −2.64E+16 |
| CD.NK.CCL3.CD160 | RPL9 | 1.74E−21 | 0.4132476 | 0.867 | 0.784 | 4.01E−17 | 1.03E+16 |
| CD.Plsma/IgA.IGLL5.IGLV2-14 | RPS3A | 1.98E−21 | 0.434075067 | 0.992 | 0.977 | 4.55E−17 | 9.53E+15 |
| CD.Plsma/IgA.IGLV2-14.IGLV3-19 | RPS27A | 2.08E−21 | 0.84009852 | 0.994 | 0.956 | 4.79E−17 | 1.76E+16 |
| CD.Plsma/IgA.AL9287683.RPL39 | REG3A | 2.10E−21 | −0.306453987 | 0.015 | 0.211 | 4.82E−17 | −6.35E+15 |
| CD.Plsma/IgA.IGLC3.IGLC2.2 | TPT1 | 2.14E−21 | −0.497558884 | 0.995 | 0.995 | 4.94E−17 | −1.01E+16 |
| CD.Plsma/IgM.IGHM.IGLV2-14 | MT-CO3 | 2.20E−21 | 0.605337958 | 0.96 | 0.97 | 5.06E−17 | 1.20E+16 |
| CD.T.CCL20.RORA | MT-ND3 | 2.27E−21 | 0.588999477 | 0.867 | 0.677 | 5.23E−17 | 1.13E+16 |
| CD.Plsma/IgA.IGHA2.IGKV1-12 | RPL18A | 2.28E−21 | 0.295386574 | 0.977 | 0.993 | 5.24E−17 | 5.64E+15 |
| CD.T.CD8B.TRGC2 | IGLC2 | 2.41E−21 | −0.516482173 | 0.371 | 0.631 | 5.55E−17 | −9.31E+15 |
| CD.NK.CCL3.CD160 | RGCC | 2.65E−21 | 0.54054654 | 0.588 | 0.372 | 6.11E−17 | 8.85E+15 |
| CD.Plsma/IgA.IGHA2.IGKV1-12 | HSPA1A | 2.67E−21 | 0.815464064 | 0.78 | 0.559 | 6.14E−17 | 1.33E+16 |
| CD.T.CCR7.GPR183 | ZFP36 | 2.90E−21 | 0.447081487 | 0.331 | 0.164 | 6.68E−17 | 6.69E+15 |
| CD.Mono.CXCL10.TNF | FAM26F | 3.00E−21 | 1.178995546 | 0.866 | 0.634 | 6.91E−17 | 1.71E+16 |
| CD.Mac.APOE.PTGDS | IGSF6 | 3.02E−21 | −0.429516235 | 0.613 | 0.764 | 6.96E−17 | −6.17E+15 |
| CD.T.CD8B.TRGC2 | DDX3Y | 4.01E−21 | −0.502122351 | 0.003 | 0.101 | 9.22E−17 | −5.44E+15 |
| CD.Plsma/IgA.AL9287683.RPL39 | SERP1 | 4.15E−21 | −0.322883193 | 0.937 | 0.928 | 9.56E−17 | −3.38E+15 |
| CD.Plsma/IgA.IGHA2.IGKV1-12 | RPS14 | 4.36E−21 | 0.338011138 | 0.968 | 0.973 | 1.00E−16 | 3.37E+15 |
| CD.Paneth.DEFA6.ITLN2 | PRAP1 | 4.52E−21 | 0.803041118 | 0.483 | 0.087 | 1.04E−16 | 7.72E+15 |
| CD.Plsma/IgM.IGHM.JCHAIN | RPL32 | 4.85E−21 | 0.312557597 | 0.966 | 0.965 | 1.12E−16 | 2.80E+15 |
| CD.Mstcl.CTSG.CD69 | RPS29 | 5.05E−21 | 0.656444372 | 0.914 | 0.63 | 1.16E−16 | 5.65E+15 |
| CD.NK.CCL3.CD160 | KLRC1 | 5.52E−21 | −0.606558945 | 0.267 | 0.472 | 1.27E−16 | −4.78E+15 |
| CD.NK.CCL3.CD160 | HSP90AB1 | 5.94E−21 | 0.540221215 | 0.757 | 0.623 | 1.37E−16 | 3.95E+15 |
| CD.Mac.APOE.PTGDS | NEAT1 | 5.98E−21 | −0.419547338 | 0.48 | 0.691 | 1.38E−16 | −3.05E+15 |
| CD.Plsma/IgA.IGLC3.IGLC2.2 | REG1A | 6.21E−21 | −0.627345852 | 0.07 | 0.49 | 1.43E−16 | −4.39E+15 |
| CD.Plsma/IgA.AL9287683.RPL39 | MZT2B | 6.38E−21 | −0.418386366 | 0.603 | 0.721 | 1.47E−16 | −2.85E+15 |
| CD.Fibro.TFPI2.CCL13 | IFITM1 | 6.92E−21 | 1.370016182 | 0.726 | 0.258 | 1.59E−16 | 8.60E+15 |
| CD.Plsma/IgA.IGLC3.IGLC2.2 | RPS4Y1 | 7.38E−21 | −0.589903491 | 0.07 | 0.515 | 1.70E−16 | −3.47E+15 |
| CD.T.KLRB1.LTF | MT-ND3 | 7.47E−21 | 0.366141631 | 0.961 | 0.893 | 1.72E−16 | 2.13E+15 |
| CD.B.MT-CO2.MT-CO1 | RPS19 | 7.89E−21 | −0.343207181 | 0.962 | 0.989 | 1.82E−16 | −1.89E+15 |
| CD.Plsma/IgA.IGHA2.IGKV1-12 | RPS5 | 7.91E−21 | 0.363298838 | 0.958 | 0.956 | 1.82E−16 | 2.00E+15 |
| CD.Fibro.TFPI2.CCL13 | RPS13 | 7.93E−21 | 0.563257338 | 0.992 | 0.968 | 1.83E−16 | 3.09E+15 |
| CD.Plsma/IgA.HSPA1B.HSPA1A | TPT1 | 8.03E−21 | 0.605220736 | 0.983 | 0.993 | 1.85E−16 | 3.27E+15 |
| CD.Prolif/EC.UBE2C.CCNB2 | IGHA2 | 8.42E−21 | 1.08303358 | 0.706 | 0.214 | 1.94E−16 | 5.59E+15 |
| CD.Plsma/IgA.IGHA2.IGKV1-12 | REG1A | 1.11E−20 | 0.304224839 | 0.392 | 0.116 | 2.55E−16 | 1.19E+15 |
| CD.cDC1.CLEC9A.XCR1 | IGHA1 | 1.19E−20 | 0.783829559 | 0.994 | 0.96 | 2.74E−16 | 2.86E+15 |
| CD.Plsma/IgM.IGHM.IGLV2-14 | MT-ND1 | 1.43E−20 | 0.640819929 | 0.93 | 0.898 | 3.30E−16 | 1.94E+15 |
| CD.Plsma/IgA.IGLC3.IGLC2.2 | RPS26 | 1.49E−20 | 0.512296129 | 0.984 | 0.835 | 3.44E−16 | 1.49E+15 |
| CD.T.CCL4.GMZK | XCL2 | 1.51E−20 | 0.829188075 | 0.18 | 0.03 | 3.47E−16 | 2.39E+15 |
| CD.T.CCR7.SELL | REG1A | 1.55E−20 | −0.766585556 | 0.107 | 0.243 | 3.56E−16 | −2.15E+15 |
| CD.Plsma/IgA.IGKV1-12.IGHA2 | RPS27A | 1.59E−20 | 0.784513473 | 0.983 | 1 | 3.67E−16 | 2.14E+15 |
| CD.Plsma/IgM.IGHM.IGLV2-14 | NACA | 1.72E−20 | 0.723795083 | 0.871 | 0.752 | 3.97E−16 | 1.82E+15 |
| CD.Plsma/IgA.AL9287683.RPL39 | HSPA1A | 1.73E−20 | 0.7567319 | 0.462 | 0.217 | 3.99E−16 | 1.90E+15 |
| CD.Plsma/IgA.IGHA2.IGKV1-12 | SEC61B | 1.84E−20 | 0.39127846 | 0.928 | 0.909 | 4.24E−16 | 9.23E+14 |
| CD.Plsma/IgA.AL9287683.RPL39 | TOMM7 | 1.92E−20 | −0.380911017 | 0.723 | 0.815 | 4.43E−16 | −8.60E+14 |
| CD.Mono.CXCL10.TNF | IGHG1 | 2.03E−20 | 1.605705979 | 0.713 | 0.314 | 4.68E−16 | 3.43E+15 |
| CD.T.CCL20.RORA | PTPRC | 2.35E−20 | −0.502748934 | 0.783 | 0.891 | 5.40E−16 | −9.31E+14 |
| CD.B.MT-CO2.MT-CO1 | TPT1 | 2.45E−20 | 0.689765843 | 0.876 | 0.816 | 5.65E−16 | 1.22E+15 |
| CD.Plsma/IgA.AL9287683.RPL39 | RPS18 | 2.53E−20 | −0.284036799 | 0.996 | 0.995 | 5.83E−16 | −4.87E+14 |
| CD.B.MT-CO2.MT-CO1 | HLA-DQA2 | 2.64E−20 | 0.61167735 | 0.163 | 0.024 | 6.08E−16 | 1.01E+15 |
| CD.Plsma/IgA.IGHA1.IGKC | IGHV3-23 | 2.75E−20 | −0.686729149 | 0.108 | 0.35 | 6.34E−16 | −1.08E+15 |
| CD.NK.CCL3.CD160 | HOPX | 2.96E−20 | 0.524031331 | 0.472 | 0.25 | 6.81E−16 | 7.70E+14 |
| CD.T.TRDC.TRGC2 | RPL39 | 3.04E−20 | 0.33905492 | 0.988 | 0.965 | 7.00E−16 | 4.85E+14 |
| CD.Plsma/IgM.IGHM.IGLV2-14 | RPS3A | 3.73E−20 | 0.543220197 | 0.927 | 0.914 | 8.59E−16 | 6.32E+14 |
| CD.Bclls.CD69.GPR183 | ATF4 | 3.76E−20 | 0.82312769 | 0.414 | 0.161 | 8.66E−16 | 9.50E+14 |
| CD.Plsma/IgA.IGKV1-12.IGHA2 | MT-ATP6 | 3.79E−20 | 0.916289708 | 0.975 | 0.915 | 8.73E−16 | 1.05E+15 |
| CD.B.MT-CO2.MT-CO1 | RPL37 | 3.82E−20 | 0.572013112 | 0.876 | 0.793 | 8.79E−16 | 6.51E+14 |
| CD.Endth/Cap.CD36.FLT1 | MTRNR2L8 | 3.88E−20 | 1.278757282 | 0.555 | 0.146 | 8.94E−16 | 1.43E+15 |
| CD.Mac.APOE.PTGDS | APOC1 | 3.96E−20 | −0.381657304 | 0.589 | 0.813 | 9.11E−16 | −4.19E+14 |
| CD.Prolif/EC.UBE2C.CCNB2 | IGHM | 3.97E−20 | 1.208452315 | 0.745 | 0.244 | 9.13E−16 | 1.32E+15 |
| CD.Plsma/IgA.IGHA2.IGKV1-12 | RPL22 | 4.05E−20 | 0.39788879 | 0.869 | 0.724 | 9.32E−16 | 4.27E+14 |
| CD.B.MT-CO2.MT-CO1 | IGHA1 | 4.29E−20 | 0.511793991 | 0.909 | 0.753 | 9.88E−16 | 5.18E+14 |
| CD.T.MT-CO2.MT-CO3 | RPL37 | 4.90E−20 | 0.55605514 | 0.879 | 0.698 | 1.13E−15 | 4.93E+14 |
| CD.Mono.S100A8.S100A9 | ACTB | 5.05E−20 | −0.535155275 | 1 | 0.994 | 1.16E−15 | −4.60E+14 |
| CD.Bclls.CD69.GPR183 | RPS29 | 5.15E−20 | −0.299316148 | 0.977 | 0.992 | 1.19E−15 | −2.52E+14 |
| CD.Plsma/IgM.IGHM.JCHAIN | RPL26 | 5.57E−20 | 0.292839144 | 0.932 | 0.914 | 1.28E−15 | 2.28E+14 |
| CD.T.CCL20.RORA | HLA-B | 5.94E−20 | 0.359957851 | 0.973 | 0.952 | 1.37E−15 | 2.63E+14 |
| CD.Plsma/IgA.AL9287683.RPL39 | EIF5A | 6.02E−20 | −0.379632595 | 0.423 | 0.639 | 1.39E−15 | −2.74E+14 |
| CD.NK.CCL3.CD160 | AC090498.1 | 7.84E−20 | 0.514850299 | 0.292 | 0.109 | 1.81E−15 | 2.85E+14 |
| CD.Plsma/IgM.IGHM.JCHAIN | RGS2 | 7.97E−20 | 0.574484612 | 0.807 | 0.643 | 1.84E−15 | 3.13E+14 |
| CD.Plsma/IgA.IGLV2-14.IGLV3-19 | NACA | 8.56E−20 | 0.986249046 | 0.976 | 0.735 | 1.97E−15 | 5.00E+14 |
| CD.Plsma/IgM.IGHM.IGLV2-14 | RPS23 | 8.83E−20 | 0.526918422 | 0.916 | 0.917 | 2.03E−15 | 2.59E+14 |
| CD.Bclls.CD69.GPR183 | HSP90AB1 | 9.12E−20 | 0.797130741 | 0.823 | 0.691 | 2.10E−15 | 3.80E+14 |
| CD.Plsma/IgA.AL9287683.RPL39 | RPL15 | 9.85E−20 | −0.26862845 | 0.993 | 0.992 | 2.27E−15 | −1.18E+14 |
| CD.Plsma/IgA.IGHA1.IGKC | JSRP1 | 1.06E−19 | 0.294704222 | 0.368 | 0.159 | 2.43E−15 | 1.21E+14 |
| CD.Plsma/IgA.IGHA2.IGKV1-12 | RPL24 | 1.08E−19 | 0.351491853 | 0.93 | 0.906 | 2.48E−15 | 1.42E+14 |
| CD.Plsma/IgA.IGLC3.IGLC2.1 | IGLC2 | 1.10E−19 | 0.322749352 | 0.995 | 0.998 | 2.54E−15 | 1.27E+14 |
| CD.T.CCL20.RORA | RPL39 | 1.12E−19 | 0.301275635 | 0.973 | 0.969 | 2.59E−15 | 1.16E+14 |
| CD.Plsma/IgA.IGLV2-14.IGLV3-19 | MT-CO3 | 1.16E−19 | 0.772597287 | 1 | 0.941 | 2.68E−15 | 2.89E+14 |
| CD.T.KLRB1.LTF | IGHG1 | 1.30E−19 | −0.586271319 | 0.04 | 0.179 | 3.00E−15 | −1.96E+14 |
| CD.Mito.MT-CO2.MT-ND2 | MTRNR2L8 | 1.41E−19 | 0.918229997 | 0.278 | 0.074 | 3.24E−15 | 2.83E+14 |
| CD.Paneth.DEFA6.ITLN2 | IGHM | 1.46E−19 | 0.775854914 | 0.795 | 0.327 | 3.35E−15 | 2.31E+14 |
| CD.Paneth.DEFA6.ITLN2 | ADIRF | 1.57E−19 | 0.604687865 | 0.341 | 0.024 | 3.60E−15 | 1.68E+14 |
| CD.Plsma/IgA.IGLC3.IGLC2.2 | DEFA6 | 1.59E−19 | −0.617487885 | 0.032 | 0.42 | 3.67E−15 | −1.68E+14 |
| CD.Plsma/IgA.IGHA2.IGKV1-12 | RPL11 | 1.73E−19 | 0.317268352 | 0.97 | 0.963 | 3.98E−15 | 7.97E+13 |
| CD.Plsma/IgA.IGHA1.IGKC | UBB | 1.74E−19 | −0.402232542 | 0.852 | 0.925 | 4.00E−15 | −1.01E+14 |
| CD.Plsma/IgA.AL9287683.RPL39 | UBA52 | 1.82E−19 | −0.291768816 | 0.948 | 0.964 | 4.18E−15 | −6.98E+13 |
| CD.Plsma/IgA.IGHA2.IGKV1-12 | RPL29 | 1.85E−19 | 0.348119806 | 0.943 | 0.926 | 4.25E−15 | 8.18E+13 |
| CD.Plsma/IgM.IGHM.JCHAIN | IGKV1-12 | 2.09E−19 | 1.051912486 | 0.623 | 0.378 | 4.81E−15 | 2.19E+14 |
| CD.Plsma/IgA.IGLV2-14.IGLV3-19 | RPS23 | 2.32E−19 | 0.922312551 | 0.988 | 0.868 | 5.35E−15 | 1.72E+14 |
| CD.Bclls.CD69.GPR183 | MRPL18 | 2.43E−19 | 0.738584281 | 0.353 | 0.121 | 5.60E−15 | 1.32E+14 |
| CD.cDC1.CLEC9A.XCR1 | HSPE1 | 2.46E−19 | 0.856233622 | 0.948 | 0.894 | 5.67E−15 | 1.51E+14 |
| CD.Plsma/IgM.IGHM.JCHAIN | MT-ND4 | 2.47E−19 | 0.401196508 | 0.978 | 0.982 | 5.69E−15 | 7.05E+13 |
| CD.Plsma/IgA.IGHA2.IGKV1-12 | RPL5 | 2.50E−19 | 0.43141023 | 0.898 | 0.889 | 5.75E−15 | 7.51E+13 |
| CD.NK.CCL3.CD160 | IGLC2 | 2.55E−19 | 0.530241345 | 0.649 | 0.455 | 5.87E−15 | 9.04E+13 |
| CD.B.MT-CO2.MT-CO1 | RPL37A | 2.65E−19 | 0.511749323 | 0.885 | 0.753 | 6.10E−15 | 8.39E+13 |
| CD.Plsma/IgA.AL9287683.RPL39 | RPS15A | 2.75E−19 | −0.284642281 | 0.996 | 0.995 | 6.33E−15 | −4.50E+13 |
| CD.Plsma/IgM.IGHM.JCHAIN | GLTSCR2 | 2.93E−19 | 0.339420977 | 0.753 | 0.529 | 6.75E−15 | 5.03E+13 |
| CD.Bclls.CD69.GPR183 | DNAJB6 | 2.99E−19 | 0.81068041 | 0.492 | 0.239 | 6.88E−15 | 1.18E+14 |
| CD.Fibro.TFPI2.CCL13 | HTRA3 | 3.22E−19 | −0.730188163 | 0.09 | 0.484 | 7.41E−15 | −9.86E+13 |
| CD.Plsma/IgA.IGHA1.IGKC | HLA-B | 3.23E−19 | 0.284199983 | 0.963 | 0.941 | 7.43E−15 | 3.82E+13 |
| CD.T.TRDC.TRGC2 | RPS27 | 3.26E−19 | 0.263845199 | 1 | 1 | 7.50E−15 | 3.52E+13 |
| CD.Fibro.TFPI2.CCL13 | IGFBP3 | 3.58E−19 | −1.517502804 | 0.145 | 0.559 | 8.25E−15 | −1.84E+14 |
| CD.Fibro.TFPI2.CCL13 | RPS20 | 3.74E−19 | 0.631618189 | 0.974 | 0.871 | 8.61E−15 | 7.33E+13 |
| CD.cDC1.CLEC9A.XCR1 | VIM | 3.89E−19 | 0.610070104 | 0.983 | 0.97 | 8.95E−15 | 6.82E+13 |
| CD.Plsma/IgA.IGLV2-14.IGLV3-19 | MT-ND2 | 4.36E−19 | 0.798803628 | 0.982 | 0.985 | 1.00E−14 | 7.97E+13 |
| CD.B.IGHG1.IGLC2 | IGLC2 | 4.43E−19 | 0.665876623 | 0.959 | 0.688 | 1.02E−14 | 6.53E+13 |
| CD.Fibro.TFPI2.CCL13 | COL1A1 | 4.98E−19 | −1.036024325 | 0.266 | 0.731 | 1.15E−14 | −9.03E+13 |
| CD.Plsma/IgA.IGKV1-12.IGHA2 | RPL12 | 5.57E−19 | 0.910371988 | 0.975 | 0.949 | 1.28E−14 | 7.11E+13 |
| CD.Paneth.DEFA6.ITLN2 | SPINK4 | 5.63E−19 | 0.903869436 | 0.58 | 0.169 | 1.30E−14 | 6.98E+13 |
| CD.Plsma/IgA.IGLV2-14.IGLV3-19 | IGKC | 5.75E−19 | 0.733965881 | 1 | 1 | 1.32E−14 | 5.55E+13 |
| CD.Plsma/IgA.IGHA2.IGKV1-12 | RPL4 | 5.94E−19 | 0.333961032 | 0.919 | 0.889 | 1.37E−14 | 2.44E+13 |
| CD.Plsma/IgA.IGKV1-12.IGHA2 | MT-CO2 | 6.35E−19 | 0.858277491 | 0.989 | 0.983 | 1.46E−14 | 5.87E+13 |
| CD.Plsma/IgM.IGHM.JCHAIN | RPL10A | 6.46E−19 | 0.292884626 | 0.924 | 0.882 | 1.49E−14 | 1.97E+13 |
| CD.Plsma/IgA.IGLV2-14.IGLV3-19 | RPL7 | 7.08E−19 | 0.84714345 | 0.994 | 0.912 | 1.63E−14 | 5.20E+13 |
| CD.NK.CCL3.CD160 | HSPA8 | 7.50E−19 | 0.473556862 | 0.712 | 0.556 | 1.73E−14 | 2.74E+13 |
| CD.Plsma/IgA.IGHA2.IGKV1-12 | PPP1R15A | 7.65E−19 | −0.56667225 | 0.453 | 0.658 | 1.76E−14 | −3.22E+13 |
| CD.TA.PLCG2.SOX4 | PHGR1 | 7.66E−19 | −1.051341776 | 0.551 | 0.864 | 1.76E−14 | −5.96E+13 |
| CD.Plsma/IgM.IGHM.JCHAIN | RPS9 | 7.98E−19 | 0.258497281 | 0.939 | 0.925 | 1.84E−14 | 1.41E+13 |
| CD.Plsma/IgM.IGHM.JCHAIN | RPL4 | 7.99E−19 | 0.293730178 | 0.903 | 0.857 | 1.84E−14 | 1.60E+13 |
| CD.Plsma/IgA.AL9287683.RPL39 | IGHM | 8.01E−19 | 0.581297315 | 0.797 | 0.422 | 1.84E−14 | 3.15E+13 |
| CD.Endth/Cap.CD36.FLT1 | IFITM1 | 8.41E−19 | 1.07056908 | 0.655 | 0.283 | 1.94E−14 | 5.53E+13 |
| CD.T.KLRB1.LTF | RPS10 | 8.57E−19 | 0.464911688 | 0.787 | 0.621 | 1.97E−14 | 2.36E+13 |
| CD.Plsma/IgA.IGHA1.IGKC | JCHAIN | 9.02E−19 | −0.276959043 | 1 | 0.999 | 2.08E−14 | −1.33E+13 |
| CD.Plsma/IgA.IGKV1-12.IGHA2 | MT-CO3 | 9.08E−19 | 0.672042659 | 0.983 | 0.983 | 2.09E−14 | 3.22E+13 |
| CD.T.CCL4.GMZK | ZFP36 | 9.96E−19 | 0.563296153 | 0.473 | 0.244 | 2.29E−14 | 2.46E+13 |
| CD.Fibro.TFPI2.CCL13 | SNHG25 | 1.10E−18 | −0.522085366 | 0.013 | 0.258 | 2.52E−14 | −2.07E+13 |
| CD.T.CCR7.SELL | JUNB | 1.14E−18 | −0.303792804 | 0.72 | 0.796 | 2.64E−14 | −1.15E+13 |
| CD.T.MT-CO2.MT-CO3 | IGHM | 1.17E−18 | 0.920857766 | 0.481 | 0.185 | 2.69E−14 | 3.42E+13 |
| CD.Plsma/IgA.IGHA2.IGKV1-12 | AL928768.3 | 1.19E−18 | 0.528882686 | 0.765 | 0.517 | 2.73E−14 | 1.94E+13 |
| CD.Plsma/IgA.IGHA2.IGKV1-12 | TMED2 | 1.19E−18 | −0.370959727 | 0.47 | 0.692 | 2.74E−14 | −1.35E+13 |
| CD.T.CCL4.GMZK | RPS26 | 1.20E−18 | 0.328771025 | 0.831 | 0.598 | 2.76E−14 | 1.19E+13 |
| CD.Plsma/IgA.AL9287683.RPL39 | COX7A2 | 1.20E−18 | −0.330347117 | 0.841 | 0.892 | 2.76E−14 | −1.20E+13 |
| CD.Plsma/IgA.IGHA2.IGKV1-12 | RPL6 | 1.31E−18 | 0.342593815 | 0.956 | 0.958 | 3.03E−14 | 1.13E+13 |
| CD.Fibro.TFPI2.CCL13 | BTF3 | 1.34E−18 | 0.765182858 | 0.897 | 0.785 | 3.08E−14 | 2.49E+13 |
| CD.Plsma/IgA.AL9287683.RPL39 | RPL29 | 1.44E−18 | −0.291087789 | 0.98 | 0.979 | 3.30E−14 | −8.81E+12 |
| CD.EC.MTRNR2L1.MT-ND3 | CD74 | 1.44E−18 | −0.684605505 | 0.54 | 0.729 | 3.31E−14 | −2.07E+13 |
| CD.Plsma/IgA.IGKV1-12.IGHA2 | RPL30 | 1.46E−18 | 0.776457245 | 0.961 | 0.881 | 3.36E−14 | 2.31E+13 |
| CD.T.CD8B.TRGC2 | ACTB | 1.64E−18 | 0.350379711 | 0.987 | 0.962 | 3.77E−14 | 9.29E+12 |
| CD.Plsma/IgA.IGKV1-12.IGHA2 | RPS24 | 1.64E−18 | 0.780119062 | 0.989 | 0.915 | 3.78E−14 | 2.06E+13 |
| CD.T.CCR7.GPR183 | RPS4Y1 | 1.68E−18 | −0.412980903 | 0.245 | 0.422 | 3.87E−14 | −1.07E+13 |
| CD.Mac.APOE.PTGDS | STAT1 | 1.76E−18 | −0.276109272 | 0.481 | 0.702 | 4.04E−14 | −6.83E+12 |
| CD.Plsma/IgA.IGHA2.IGKV1-12 | RPL12 | 1.79E−18 | 0.407995629 | 0.958 | 0.941 | 4.11E−14 | 9.92E+12 |
| CD.Plsma/IgA.IGHA2.IGKV1-12 | RPS9 | 1.82E−18 | 0.315589452 | 0.966 | 0.936 | 4.19E−14 | 7.53E+12 |
| CD.Plsma/IgA.IGLC3.IGLC2.2 | EIF1AY | 1.90E−18 | −0.471591095 | 0.032 | 0.41 | 4.37E−14 | −1.08E+13 |
| CD.Plsma/IgM.IGHM.IGLV2-14 | RPS27A | 1.92E−18 | 0.431840474 | 0.949 | 0.951 | 4.43E−14 | 9.75E+12 |
| CD.Plsma/IgA.IGHA2.IGKV1-12 | JUN | 1.96E−18 | −0.414833159 | 0.888 | 0.968 | 4.51E−14 | −9.19E+12 |
| CD.B.MT-CO2.MT-CO1 | TMSB4X | 1.96E−18 | −0.290885779 | 0.995 | 0.999 | 4.52E−14 | −6.44E+12 |
| CD.Plsma/IgM.IGHM.JCHAIN | SPINT2 | 2.02E−18 | 0.331221835 | 0.373 | 0.151 | 4.65E−14 | 7.12E+12 |
| CD.Plsma/IgA.IGKV1-12.IGHA2 | RPS23 | 2.09E−18 | 0.845097151 | 0.978 | 0.915 | 4.80E−14 | 1.76E+13 |
| CD.Plsma/IgA.IGKV1-12.IGHA2 | IGHM | 2.18E−18 | 1.902672111 | 0.961 | 0.678 | 5.02E−14 | 3.79E+13 |
| CD.T.MT-CO2.MT-CO3 | IGHA1 | 2.20E−18 | 0.517561696 | 0.927 | 0.757 | 5.06E−14 | 1.02E+13 |
| CD.T.CCR7.SELL | IGHA2 | 2.20E−18 | 0.306799726 | 0.376 | 0.201 | 5.07E−14 | 6.05E+12 |
| CD.Fibro.TFPI2.CCL13 | HIF1A | 2.38E−18 | −0.977155958 | 0.194 | 0.645 | 5.47E−14 | −1.79E+13 |
| CD.Mac.APOE.PTGDS | MTRNR2L8 | 2.40E−18 | 0.550899657 | 0.172 | 0.055 | 5.52E−14 | 9.98E+12 |
| CD.T.CD8B.TRGC2 | IGKC | 2.56E−18 | −0.346650057 | 0.923 | 0.984 | 5.88E−14 | −5.89E+12 |
| CD.Prolif/EC.UBE2C.CCNB2 | HLA-B | 2.61E−18 | −0.825115782 | 0.941 | 0.992 | 6.01E−14 | −1.37E+13 |
| CD.Plsma/IgA.AL9287683.RPL39 | ISG20 | 2.66E−18 | −0.359663487 | 0.843 | 0.895 | 6.12E−14 | −5.87E+12 |
| CD.Bclls.CD69.GPR183 | IER2 | 2.77E−18 | 0.850172775 | 0.534 | 0.285 | 6.39E−14 | 1.33E+13 |
| CD.T.CD8B.TRGC2 | MT-ND3 | 3.10E−18 | 0.29079908 | 0.984 | 0.967 | 7.13E−14 | 4.08E+12 |
| CD.T.CCL20.RORA | MT-ND1 | 3.44E−18 | 0.586533361 | 0.779 | 0.581 | 7.92E−14 | 7.40E+12 |
| CD.Mac.APOE.PTGDS | VAMP5 | 3.58E−18 | −0.311180325 | 0.434 | 0.635 | 8.23E−14 | −3.78E+12 |
| CD.NK.CCL3.CD160 | EIF1 | 3.93E−18 | 0.258453968 | 0.945 | 0.896 | 9.05E−14 | 2.86E+12 |
| CD.Plsma/IgA.IGHA1.IGKC | HSP90AB1 | 4.05E−18 | −0.407624774 | 0.595 | 0.761 | 9.33E−14 | −4.37E+12 |
| CD.B.RPS29.RPS21 | XIST | 4.14E−18 | 0.410894747 | 0.23 | 0.058 | 9.54E−14 | 4.31E+12 |
| CD.Bclls.CD69.GPR183 | EGR1 | 4.20E−18 | 0.899942905 | 0.207 | 0.046 | 9.67E−14 | 9.30E+12 |
| CD.T.TRDC.TRGC2 | RPL41 | 4.41E−18 | 0.286053363 | 0.99 | 0.965 | 1.01E−13 | 2.82E+12 |
| CD.NK.CCL3.CD160 | MTRNR2L8 | 5.09E−18 | 0.618162542 | 0.12 | 0.015 | 1.17E−13 | 5.27E+12 |
| CD.Mac.APOE.PTGDS | RNASE1 | 5.24E−18 | 0.943671019 | 0.106 | 0.02 | 1.21E−13 | 7.83E+12 |
| CD.Plsma/IgA.IGHA1.IGKC | HSPA1A | 5.31E−18 | −0.561611439 | 0.386 | 0.6 | 1.22E−13 | −4.60E+12 |
| CD.Bclls.CD69.GPR183 | MT-ND1 | 5.50E−18 | 0.527040812 | 0.85 | 0.73 | 1.27E−13 | 4.16E+12 |
| CD.Plsma/IgG.IGHGP.IGHG2 | CADM1 | 5.59E−18 | 0.26802707 | 0.405 | 0.281 | 1.29E−13 | 2.08E+12 |
| CD.Plsma/IgA.IGHA2.IGKV1-12 | SOCS3 | 5.63E−18 | −0.423262071 | 0.316 | 0.552 | 1.30E−13 | −3.27E+12 |
| CD.Plsma/IgA.IGHA1.IGKC | PPP1R15A | 5.90E−18 | −0.46193948 | 0.503 | 0.7 | 1.36E−13 | −3.40E+12 |
| CD.Plsma/IgA.IGHA2.IGKV1-12 | CIRBP | 6.09E−18 | 0.43416559 | 0.837 | 0.695 | 1.40E−13 | 3.09E+12 |
| CD.Fibro.TFPI2.CCL13 | SLC16A4 | 6.21E−18 | −0.754569078 | 0.078 | 0.441 | 1.43E−13 | −5.28E+12 |
| CD.cDC1.CLEC9A.XCR1 | HLA-DRB5 | 6.45E−18 | 0.845214702 | 0.89 | 0.5 | 1.48E−13 | 5.70E+12 |
| CD.Plsma/IgA.AL9287683.RPL39 | RPL5 | 6.62E−18 | −0.280092349 | 0.972 | 0.974 | 1.52E−13 | −1.84E+12 |
| CD.B.RPS29.RPS21 | HLA-DRB5 | 6.81E−18 | 0.474839681 | 0.61 | 0.438 | 1.57E−13 | 3.03E+12 |
| CD.Plsma/IgM.IGHM.JCHAIN | HERPUD1 | 6.88E−18 | 0.299964661 | 0.975 | 0.971 | 1.58E−13 | 1.89E+12 |
| CD.cDC1.CLEC9A.XCR1 | TPT1 | 7.33E−18 | 0.651533421 | 1 | 1 | 1.69E−13 | 3.86E+12 |
| CD.EC.MTRNR2L1.MT-ND3 | MT-ATP6 | 7.41E−18 | 0.330686386 | 1 | 1 | 1.71E−13 | 1.94E+12 |
| CD.Plsma/IgA.IGHA1.IGKC | ANKRD28 | 8.80E−18 | −0.380091553 | 0.55 | 0.743 | 2.03E−13 | −1.88E+12 |
| CD.EC.MT1H.MT1G | IFI27 | 9.97E−18 | −1.592731165 | 0.052 | 0.639 | 2.30E−13 | −6.94E+12 |
| CD.Plsma/IgA.HSPA1B.HSPA1A | RPL30 | 1.00E−17 | 0.463845995 | 0.944 | 0.954 | 2.30E−13 | 2.02E+12 |
| CD.Plsma/IgM.IGHM.JCHAIN | RPL6 | 1.07E−17 | 0.279519427 | 0.944 | 0.957 | 2.46E−13 | 1.14E+12 |
| CD.Plsma/IgA.IGHA2.IGKV1-12 | RPL32 | 1.10E−17 | 0.327095674 | 0.964 | 0.988 | 2.53E−13 | 1.30E+12 |
| CD.Plsma/IgA.IGLL5.IGLV2-14 | MT-ND2 | 1.12E−17 | 0.408139243 | 0.987 | 0.989 | 2.59E−13 | 1.58E+12 |
| CD.T.CCR7.GPR183 | DEFA6 | 1.15E−17 | −0.873903024 | 0.056 | 0.171 | 2.64E−13 | −3.31E+12 |
| CD.Plsma/IgA.IGKV1-12.IGHA2 | RPL13 | 1.16E−17 | 0.665701695 | 0.994 | 0.983 | 2.66E−13 | 2.50E+12 |
| CD.Plsma/IgM.IGHM.JCHAIN | RPL39 | 1.19E−17 | 0.292698253 | 0.948 | 0.957 | 2.75E−13 | 1.06E+12 |
| CD.T.CD8B.TRGC2 | RPL8 | 1.23E−17 | −0.275412383 | 0.932 | 0.962 | 2.84E−13 | −9.71E+11 |
| CD.Plsma/IgA.IGLC3.IGLC2.2 | FTL | 1.32E−17 | −0.407033281 | 0.989 | 1 | 3.04E−13 | −1.34E+12 |
| CD.Paneth.DEFA6.ITLN2 | PHGR1 | 1.34E−17 | 0.754512365 | 0.62 | 0.232 | 3.09E−13 | 2.44E+12 |
| CD.cDC1.CLEC9A.XCR1 | MT-ND1 | 1.35E−17 | 0.542936461 | 0.988 | 0.965 | 3.11E−13 | 1.75E+12 |
| CD.Plsma/IgM.IGHM.JCHAIN | RPS7 | 1.35E−17 | 0.273630476 | 0.926 | 0.912 | 3.11E−13 | 8.79E+11 |
| CD.T.KLRB1.LTF | XIST | 1.36E−17 | 0.824655849 | 0.261 | 0.028 | 3.12E−13 | 2.64E+12 |
| CD.Fibro.TFPI2.CCL13 | FCER1G | 1.36E−17 | −0.674472146 | 0.111 | 0.516 | 3.13E−13 | −2.16E+12 |
| CD.T.KLRB1.LTF | MT-CO3 | 1.45E−17 | 0.302043904 | 0.976 | 0.934 | 3.34E−13 | 9.05E+11 |
| CD.Plsma/IgG.IGHGP.IGHG2 | HSPA1B | 1.50E−17 | −0.368950784 | 0.251 | 0.398 | 3.45E−13 | −1.07E+12 |
| CD.Plsma/IgA.IGLV2-14.IGLV3-19 | EEF1B2 | 1.54E−17 | 0.804742594 | 0.952 | 0.779 | 3.54E−13 | 2.27E+12 |
| CD.Mac.APOE.PTGDS | SEPP1 | 1.59E−17 | 0.351604076 | 0.852 | 0.731 | 3.67E−13 | 9.58E+11 |
| CD.Plsma/IgA.IGLV2-14.IGLV3-19 | IGLL5 | 1.60E−17 | 0.899735607 | 0.868 | 0.338 | 3.67E−13 | 2.45E+12 |
| CD.NK.CCL3.CD160 | MT-ND3 | 1.62E−17 | 0.295042902 | 0.907 | 0.803 | 3.73E−13 | 7.92E+11 |
| CD.NK.CCL3.CD160 | DNAJA1 | 1.69E−17 | 0.548386935 | 0.646 | 0.487 | 3.90E−13 | 1.41E+12 |
| CD.Fibro.TFPI2.CCL13 | IGHG3 | 1.72E−17 | −0.842892593 | 0.054 | 0.376 | 3.95E−13 | −2.13E+12 |
| CD.Plsma/IgM.IGHM.JCHAIN | RPS17 | 1.76E−17 | 0.298353788 | 0.932 | 0.935 | 4.05E−13 | 7.37E+11 |
| CD.Paneth.DEFA6.ITLN2 | RP11-1143G9.4 | 1.79E−17 | 0.938061394 | 0.732 | 0.417 | 4.11E−13 | 2.28E+12 |
| CD.Fibro.TFPI2.CCL13 | RPS6 | 1.87E−17 | 0.397870514 | 0.997 | 0.978 | 4.30E−13 | 9.26E+11 |
| CD.B.RPS29.RPS21 | HLA-DQA2 | 2.00E−17 | −0.534783677 | 0.116 | 0.28 | 4.59E−13 | −1.16E+12 |
| CD.Plsma/IgA.AL9287683.RPL39 | FKBP2 | 2.00E−17 | −0.307272297 | 0.915 | 0.94 | 4.60E−13 | −6.69E+11 |
| CD.NK.CCL3.CD160 | EEF1B2 | 2.03E−17 | 0.443811192 | 0.637 | 0.462 | 4.68E−13 | 9.49E+11 |
| CD.Mono.CXCL10.TNF | IGHA2 | 2.29E−17 | 2.982390972 | 0.573 | 0.157 | 5.27E−13 | 5.66E+12 |
| CD.T.CCL20.RORA | RPS29 | 2.39E−17 | 0.400088613 | 0.962 | 0.938 | 5.50E−13 | 7.28E+11 |
| CD.Plsma/IgM.IGHM.JCHAIN | UQCRB | 2.59E−17 | 0.338281454 | 0.773 | 0.594 | 5.96E−13 | 5.67E+11 |
| CD.Mac.APOE.PTGDS | C1QC | 2.86E−17 | 0.321505807 | 0.898 | 0.915 | 6.57E−13 | 4.89E+11 |
| CD.Plsma/IgA.IGHA2.IGKV1-12 | IGKV1-5 | 2.93E−17 | 0.406793822 | 0.581 | 0.276 | 6.74E−13 | 6.04E+11 |
| CD.cDC1.CLEC9A.XCR1 | HSPD1 | 2.95E−17 | 0.855712315 | 0.931 | 0.788 | 6.79E−13 | 1.26E+12 |
| CD.NK.CCL3.CD160 | RPS2 | 2.99E−17 | −0.258099414 | 0.908 | 0.93 | 6.89E−13 | −3.75E+11 |
| CD.Plsma/IgA.IGLL5.IGLV2-14 | RPL30 | 3.09E−17 | 0.377221692 | 0.979 | 0.928 | 7.10E−13 | 5.31E+11 |
| CD.Plsma/IgM.IGHM.JCHAIN | RPL24 | 3.29E−17 | 0.335670328 | 0.917 | 0.884 | 7.57E−13 | 4.43E+11 |
| CD.Plsma/IgA.IGHA1.IGKC | RPS10 | 3.39E−17 | 0.305920564 | 0.746 | 0.573 | 7.80E−13 | 3.92E+11 |
| CD.Plsma/IgA.IGLV2-14.IGLV3-19 | RPL30 | 3.50E−17 | 0.771340049 | 0.97 | 0.779 | 8.05E−13 | 9.58E+11 |
| CD.Plsma/IgA.IGLV2-14.IGLV3-19 | MT-ATP6 | 3.51E−17 | 0.84466797 | 0.97 | 0.853 | 8.08E−13 | 1.05E+12 |
| CD.Fibro.TFPI2.CCL13 | MT-CYB | 3.54E−17 | 0.422423071 | 0.995 | 0.968 | 8.14E−13 | 5.19E+11 |
| CD.TA.PLCG2.SOX4 | MT-ND3 | 3.61E−17 | 0.481820002 | 0.987 | 0.97 | 8.31E−13 | 5.80E+11 |
| CD.T.CARD16.GB2 | MT-ATP6 | 3.61E−17 | 0.454136298 | 0.87 | 0.626 | 8.32E−13 | 5.46E+11 |
| CD.Fibro.TFPI2.CCL13 | SERPINE2 | 3.72E−17 | −1.269469223 | 0.326 | 0.72 | 8.56E−13 | −1.48E+12 |
| CD.T.CD8B.TRGC2 | EIF5A | 4.32E−17 | −0.458021261 | 0.184 | 0.407 | 9.95E−13 | −4.60E+11 |
| CD.Fibro.TFPI2.CCL13 | ROMO1 | 4.33E−17 | −0.739586064 | 0.145 | 0.559 | 9.96E−13 | −7.42E+11 |
| CD.Fibro.TFPI2.CCL13 | CAPZA1 | 4.75E−17 | −0.67984193 | 0.096 | 0.473 | 1.09E−12 | −6.22E+11 |
| CD.Plsma/IgA.HSPA1B.HSPA1A | VIM | 4.81E−17 | 0.648637203 | 0.906 | 0.821 | 1.11E−12 | 5.86E+11 |
| CD.Plsma/IgM.IGHM.JCHAIN | RPL18 | 5.06E−17 | 0.267485685 | 0.941 | 0.933 | 1.16E−12 | 2.30E+11 |
| CD.Plsma/IgA.AL9287683.RPL39 | RPS14 | 5.26E−17 | −0.258481314 | 0.998 | 0.997 | 1.21E−12 | −2.13E+11 |
| CD.Mito.MT-CO2.MT-ND2 | CCL4L2 | 5.31E−17 | 1.049197663 | 0.415 | 0.203 | 1.22E−12 | 8.58E+11 |
| CD.Bclls.CD69.GPR183 | EIF5 | 5.37E−17 | 0.706831503 | 0.526 | 0.283 | 1.24E−12 | 5.72E+11 |
| CD.Plsma/IgA.IGLC3.IGLC2.2 | DUSP5 | 5.44E−17 | −0.658191977 | 0.758 | 0.88 | 1.25E−12 | −5.26E+11 |
| CD.Mac.APOE.PTGDS | MT-ND4L | 5.46E−17 | −0.447027936 | 0.392 | 0.57 | 1.26E−12 | −3.56E+11 |
| CD.Plsma/IgA.IGLL5.IGLV2-14 | RPS23 | 5.85E−17 | 0.356920568 | 0.996 | 0.985 | 1.35E−12 | 2.65E+11 |
| CD.Plsma/IgA.IGLC3.IGLC2.1 | MT-ND4L | 6.03E−17 | −0.522473606 | 0.284 | 0.599 | 1.39E−12 | −3.77E+11 |
| CD.Plsma/IgA.HSPA1B.HSPA1A | MT-ND1 | 6.04E−17 | 0.594021164 | 0.965 | 0.947 | 1.39E−12 | 4.27E+11 |
| CD.Fibro.TFPI2.CCL13 | CXCL14 | 6.05E−17 | 0.777766793 | 0.979 | 0.796 | 1.39E−12 | 5.59E+11 |
| CD.Prolif/EC.UBE2C.CCNB2 | MTRNR2L8 | 6.07E−17 | 0.992397761 | 0.549 | 0.142 | 1.40E−12 | 7.10E+11 |
| CD.Plsma/IgA.IGHA2.IGKV1-12 | SEC11C | 6.28E−17 | 0.365302585 | 0.922 | 0.872 | 1.45E−12 | 2.53E+11 |
| CD.Mac.APOE.PTGDS | SAMHD1 | 6.48E−17 | −0.308775283 | 0.374 | 0.576 | 1.49E−12 | −2.07E+11 |
| CD.Plsma/IgM.IGHM.JCHAIN | MTRNR2L8 | 6.75E−17 | 0.362983533 | 0.327 | 0.129 | 1.55E−12 | 2.34E+11 |
| CD.T.CARD16.GB2 | RPS10 | 6.89E−17 | 0.601720172 | 0.674 | 0.393 | 1.59E−12 | 3.80E+11 |
| CD.Plsma/IgA.AL9287683.RPL39 | KLF2 | 7.24E−17 | −0.501466774 | 0.235 | 0.444 | 1.67E−12 | −3.01E+11 |
| CD.Plsma/IgA.IGLC3.IGLC2.1 | XIST | 7.65E−17 | −0.548542416 | 0.055 | 0.366 | 1.76E−12 | −3.11E+11 |
| CD.Mac.APOE.PTGDS | BASP1 | 7.85E−17 | −0.286699685 | 0.115 | 0.292 | 1.81E−12 | −1.59E+11 |
| CD.Fibro.TFPI2.CCL13 | CCL11 | 7.88E−17 | −0.865291325 | 0.938 | 0.978 | 1.81E−12 | −4.77E+11 |
| CD.T.KLRB1.LTF | IGKC | 8.21E−17 | −0.289194282 | 0.894 | 0.962 | 1.89E−12 | −1.53E+11 |
| CD.Plsma/IgA.IGHA1.IGKC | IGHA2 | 8.30E−17 | −0.295144163 | 0.955 | 0.979 | 1.91E−12 | −1.54E+11 |
| CD.Plsma/IgA.IGHA2.IGKV1-12 | RPL8 | 8.39E−17 | 0.282219297 | 0.977 | 0.958 | 1.93E−12 | 1.46E+11 |
| CD.Mac.APOE.PTGDS | TAX1BP1 | 8.74E−17 | −0.252468756 | 0.39 | 0.576 | 2.01E−12 | −1.26E+11 |
| CD.T.CD8B.TRGC2 | IL32 | 9.11E−17 | 0.371125205 | 0.886 | 0.8 | 2.10E−12 | 1.77E+11 |
| CD.Plsma/IgA.IGHA1.IGKC | MT-CO3 | 9.21E−17 | 0.255569901 | 0.926 | 0.925 | 2.12E−12 | 1.21E+11 |
| CD.Mac.APOE.PTGDS | ALOX5AP | 9.30E−17 | 0.372598209 | 0.14 | 0.039 | 2.14E−12 | 1.74E+11 |
| CD.Plsma/IgA.IGLV2-14.IGLV3-19 | MT-CYB | 9.34E−17 | 0.749547123 | 0.982 | 0.941 | 2.15E−12 | 3.49E+11 |
| CD.T.CCR7.SELL | RP11-138A9.2 | 9.77E−17 | 0.303620592 | 0.106 | 0.03 | 2.25E−12 | 1.35E+11 |
| CD.T.CCL20.RORA | HOPX | 1.03E−16 | 0.748859499 | 0.335 | 0.12 | 2.38E−12 | 3.15E+11 |
| CD.Plsma/IgG.IGHGP.IGHG2 | IGKV4-1 | 1.23E−16 | −0.484160958 | 0.174 | 0.301 | 2.83E−12 | −1.71E+11 |
| CD.Plsma/IgA.IGKV1-12.IGHA2 | RPL10 | 1.25E−16 | 0.64460602 | 1 | 1 | 2.89E−12 | 2.23E+11 |
| CD.Plsma/IgM.IGHM.JCHAIN | RPS5 | 1.30E−16 | 0.272945596 | 0.938 | 0.933 | 3.00E−12 | 9.10E+10 |
| CD.Plsma/IgA.IGHA2.IGKV1-12 | CD48 | 1.39E−16 | 0.319113114 | 0.356 | 0.116 | 3.19E−12 | 1.00E+11 |
| CD.T.CCL20.RORA | TPT1 | 1.43E−16 | 0.588256516 | 0.951 | 0.918 | 3.30E−12 | 1.78E+11 |
| CD.Plsma/IgA.IGHA2.IGKV1-12 | HLA-DRA | 1.45E−16 | 0.385954731 | 0.602 | 0.33 | 3.34E−12 | 1.16E+11 |
| CD.Plsma/IgM.IGHM.IGLV2-14 | CD48 | 1.50E−16 | 0.542560642 | 0.461 | 0.154 | 3.46E−12 | 1.57E+11 |
| CD.Mac.APOE.PTGDS | TXN | 1.52E−16 | −0.417758346 | 0.535 | 0.708 | 3.49E−12 | −1.20E+11 |
| CD.Plsma/IgM.IGHM.JCHAIN | TMSB4X | 1.53E−16 | 0.303090853 | 0.961 | 0.914 | 3.52E−12 | 8.61E+10 |
| CD.Bclls.CD69.GPR183 | RPS2 | 1.57E−16 | −0.254361963 | 0.989 | 0.995 | 3.62E−12 | −7.03E+10 |
| CD.Plsma/IgA.IGKV1-12.IGHA2 | SEC61B | 1.61E−16 | 0.65737969 | 0.964 | 0.949 | 3.70E−12 | 1.78E+11 |
| CD.Plsma/IgA.IGHA2.IGKV1-12 | EEF1D | 1.61E−16 | 0.313792976 | 0.958 | 0.936 | 3.70E−12 | 8.48E+10 |
| CD.Plsma/IgA.IGHA2.IGKV1-12 | SPCS1 | 1.62E−16 | 0.336323176 | 0.919 | 0.906 | 3.72E−12 | 9.04E+10 |
| CD.Mono.S100A8.S100A9 | RPL21 | 1.67E−16 | 0.42787595 | 0.99 | 0.965 | 3.84E−12 | 1.11E+11 |
| CD.Plsma/IgA.IGKV1-12.IGHA2 | IGHV1-3 | 1.68E−16 | −0.793134501 | 0.008 | 0.237 | 3.86E−12 | −2.05E+11 |
| CD.Plsma/IgA.IGLV2-14.IGLV3-19 | RPS20 | 1.72E−16 | 0.788551673 | 0.964 | 0.853 | 3.96E−12 | 1.99E+11 |
| CD.Bclls.CD69.GPR183 | RPS28 | 1.73E−16 | −0.273256283 | 0.977 | 0.982 | 3.97E−12 | −6.88E+10 |
| CD.Bclls.CD69.GPR183 | H3F3B | 1.75E−16 | 0.419456101 | 0.94 | 0.861 | 4.03E−12 | 1.04E+11 |
| CD.Plsma/IgA.IGLC3.IGLC2.2 | RPS12 | 1.94E−16 | −0.45442696 | 0.995 | 1 | 4.46E−12 | −1.02E+11 |
| CD.Bclls.CD69.GPR183 | UBB | 2.01E−16 | 0.527090561 | 0.88 | 0.776 | 4.64E−12 | 1.14E+11 |
| CD.Plsma/IgA.IGKV1-12.IGHA2 | RPL28 | 2.17E−16 | 0.680053924 | 0.978 | 0.915 | 4.99E−12 | 1.36E+11 |
| CD.Plsma/IgM.IGHM.JCHAIN | RPL37 | 2.18E−16 | 0.352019497 | 0.924 | 0.875 | 5.01E−12 | 7.03E+10 |
| CD.Plsma/IgA.IGLV2-14.IGLV3-19 | IGHM | 2.27E−16 | 0.73464384 | 0.982 | 0.765 | 5.22E−12 | 1.41E+11 |
| CD.Mac.APOE.PTGDS | EFHD2 | 2.30E−16 | −0.426668468 | 0.268 | 0.444 | 5.30E−12 | −8.06E+10 |
| CD.Fibro.TFPI2.CCL13 | RPL5 | 2.32E−16 | 0.600968534 | 0.961 | 0.86 | 5.35E−12 | 1.12E+11 |
| CD.Plsma/IgA.IGKV1-12.IGHA2 | RPL13A | 2.41E−16 | 0.663682749 | 0.992 | 1 | 5.55E−12 | 1.19E+11 |
| CD.Plsma/IgM.IGHM.IGLV2-14 | EEF1B2 | 2.46E−16 | 0.543837315 | 0.857 | 0.767 | 5.66E−12 | 9.62E+10 |
| CD.Fibro.TFPI2.CCL13 | RPL13A | 2.48E−16 | 0.328518692 | 0.997 | 0.989 | 5.71E−12 | 5.75E+10 |
| CD.Plsma/IgA.IGKV1-12.IGHA2 | HSP90AA1 | 2.62E−16 | 1.661378065 | 0.933 | 0.78 | 6.03E−12 | 2.75E+11 |
| CD.Mac.APOE.PTGDS | GPR65 | 2.83E−16 | −0.340836873 | 0.156 | 0.325 | 6.52E−12 | −5.23E+10 |
| CD.Fibro.TFPI2.CCL13 | ADH1B | 2.95E−16 | −0.690653801 | 0.749 | 0.925 | 6.78E−12 | −1.02E+11 |
| CD.Plsma/IgM.IGHM.JCHAIN | RPL12 | 3.19E−16 | 0.283195151 | 0.948 | 0.951 | 7.34E−12 | 3.86E+10 |
| CD.Mac.APOE.PTGDS | RPL41 | 3.23E−16 | 0.261109077 | 0.93 | 0.942 | 7.43E−12 | 3.52E+10 |
| CD.Plsma/IgA.IGHA1.IGLV6-57 | TPT1 | 3.29E−16 | 0.755246578 | 0.991 | 0.969 | 7.57E−12 | 9.98E+10 |
| CD.Plsma/IgM.IGHM.JCHAIN | RPL14 | 3.42E−16 | 0.314856606 | 0.909 | 0.886 | 7.86E−12 | 4.00E+10 |
| CD.NK.CCL3.CD160 | DYNLL1 | 3.47E−16 | 0.506419558 | 0.585 | 0.415 | 7.98E−12 | 6.35E+10 |
| CD.Plsma/IgA.IGHA2.IGKV1-12 | RPL10A | 4.14E−16 | 0.301349121 | 0.928 | 0.892 | 9.53E−12 | 3.16E+10 |
| CD.Fibro.TFPI2.CCL13 | MSRB2 | 4.42E−16 | −0.715698815 | 0.152 | 0.57 | 1.02E−11 | −7.03E+10 |
| CD.T.CD8B.TRGC2 | RP11-347P5.1 | 4.44E−16 | −0.526263521 | 0.043 | 0.174 | 1.02E−11 | −5.15E+10 |
| CD.Plsma/IgA.IGLC3.IGLC2.2 | RPL8 | 4.55E−16 | −0.397362649 | 0.995 | 0.995 | 1.05E−11 | −3.79E+10 |
| CD.Mstcl.CTSG.CD69 | RPS26 | 4.79E−16 | 0.676215316 | 0.709 | 0.332 | 1.10E−11 | 6.14E+10 |
| CD.Plsma/IgA.IGHA1.IGKC | GSTP1 | 5.15E−16 | −0.287607012 | 0.69 | 0.854 | 1.19E−11 | −2.42E+10 |
| CD.Plsma/IgA.IGHA2.IGKV1-12 | IRF1 | 5.23E−16 | −0.470867179 | 0.453 | 0.67 | 1.20E−11 | −3.91E+10 |
| CD.Mac.APOE.PTGDS | TLN1 | 5.47E−16 | −0.343921834 | 0.249 | 0.419 | 1.26E−11 | −2.73E+10 |
| CD.Plsma/IgA.IGHA1.IGKC | IGKV4-1 | 5.61E−16 | −1.018509528 | 0.161 | 0.385 | 1.29E−11 | −7.88E+10 |
| CD.B.MT-ND4L.IGHG1 | HLA-DRB5 | 5.64E−16 | −1.296709935 | 0.164 | 0.729 | 1.30E−11 | −9.99E+10 |
| CD.Mac.APOE.PTGDS | RGS2 | 5.72E−16 | −0.321799018 | 0.485 | 0.7 | 1.32E−11 | −2.44E+10 |
| CD.B.MT-ND4L.IGHG1 | CCL3 | 5.74E−16 | 0.885673427 | 0.119 | 0.009 | 1.32E−11 | 6.71E+10 |
| CD.T.CCR7.GPR183 | PABPC1 | 5.74E−16 | 0.333131458 | 0.787 | 0.661 | 1.32E−11 | 2.52E+10 |
| CD.Paneth.DEFA6.ITLN2 | RPS26 | 6.37E−16 | 0.661136344 | 0.79 | 0.433 | 1.47E−11 | 4.51E+10 |
| CD.Plsma/IgM.IGHM.IGLV2-14 | HSP90AA1 | 6.62E−16 | 0.833632489 | 0.898 | 0.868 | 1.52E−11 | 5.47E+10 |
| CD.Plsma/IgA.IGKV1-12.IGHA2 | RPS20 | 6.74E−16 | 0.746727214 | 0.958 | 0.864 | 1.55E−11 | 4.81E+10 |
| CD.T.TRDC.TRGC2 | RPS28 | 7.16E−16 | 0.296584174 | 0.983 | 0.923 | 1.65E−11 | 1.80E+10 |
| CD.Plsma/IgM.IGHM.IGLV2-14 | MT-ATP6 | 7.47E−16 | 0.386821609 | 0.93 | 0.868 | 1.72E−11 | 2.25E+10 |
| CD.Fibro.TFPI2.CCL13 | RPL6 | 7.49E−16 | 0.50144925 | 0.984 | 0.903 | 1.72E−11 | 2.91E+10 |
| CD.NK.CCL3.CD160 | CD160 | 7.74E−16 | 0.484888988 | 0.525 | 0.336 | 1.78E−11 | 2.72E+10 |
| CD.cDC1.CLEC9A.XCR1 | MRPL18 | 8.63E−16 | 0.717597404 | 0.855 | 0.641 | 1.99E−11 | 3.61E+10 |
| CD.B.MT-CO2.MT-CO1 | RPS4X | 8.83E−16 | −0.358805952 | 0.9 | 0.963 | 2.03E−11 | −1.77E+10 |
| CD.T.MT-CO2.MT-CO3 | XIST | 8.91E−16 | −1.009182817 | 0.057 | 0.302 | 2.05E−11 | −4.92E+10 |
| CD.Plsma/IgG.IGHGP.IGHG2 | RPS10 | 9.00E−16 | 0.324178703 | 0.678 | 0.613 | 2.07E−11 | 1.56E+10 |
| CD.Fibro.TFPI2.CCL13 | CEBPB | 9.16E−16 | −0.806394045 | 0.116 | 0.473 | 2.11E−11 | −3.82E+10 |
| CD.Mac.APOE.PTGDS | MTRNR2L12 | 9.35E−16 | 0.535587992 | 0.187 | 0.072 | 2.15E−11 | 2.49E+10 |
| CD.B.MT-CO2.MT-CO1 | RPS2 | 9.52E−16 | −0.300672292 | 0.962 | 0.986 | 2.19E−11 | −1.37E+10 |
| CD.Plsma/IgM.IGHM.JCHAIN | RPS11 | 9.84E−16 | 0.275015228 | 0.926 | 0.9 | 2.26E−11 | 1.21E+10 |
| CD.Plsma/IgA.IGKV1-12.IGHA2 | SSR4 | 1.02E−15 | 0.438941131 | 0.997 | 1 | 2.35E−11 | 1.87E+10 |
| CD.Plsma/IgA.IGLV2-14.IGLV3-19 | MT-ND4 | 1.02E−15 | 0.672407906 | 0.982 | 0.971 | 2.36E−11 | 2.85E+10 |
| CD.EC.MTRNR2L1.MT-ND3 | RPS2 | 1.05E−15 | −0.465957596 | 0.836 | 0.93 | 2.42E−11 | −1.93E+10 |
| CD.Plsma/IgA.AL9287683.RPL39 | COX6A1 | 1.05E−15 | −0.343872621 | 0.728 | 0.775 | 2.42E−11 | −1.42E+10 |
| CD.Plsma/IgA.IGHA2.IGKV1-12 | RRBP1 | 1.09E−15 | −0.393570017 | 0.443 | 0.64 | 2.51E−11 | −1.57E+10 |
| CD.NK.CCL3.CD160 | SERF2 | 1.11E−15 | −0.306238963 | 0.713 | 0.781 | 2.56E−11 | −1.20E+10 |
| CD.Plsma/IgA.IGHA2.IGKV1-12 | RPSA | 1.14E−15 | 0.316083964 | 0.881 | 0.825 | 2.63E−11 | 1.20E+10 |
| CD.T.TRDC.TRGC2 | RPS29 | 1.18E−15 | 0.325912899 | 0.992 | 0.92 | 2.72E−11 | 1.20E+10 |
| CD.Plsma/IgA.IGKV1-12.IGHA2 | RPS17 | 1.19E−15 | 0.631558484 | 0.966 | 0.966 | 2.73E−11 | 2.31E+10 |
| CD.cDC1.CLEC9A.XCR1 | IGHM | 1.23E−15 | 0.528635319 | 0.855 | 0.444 | 2.82E−11 | 1.87E+10 |
| CD.Plsma/IgA.IGLL5.IGLV2-14 | VIM | 1.29E−15 | 0.643376099 | 0.891 | 0.742 | 2.97E−11 | 2.17E+10 |
| CD.B.MT-CO2.MT-CO1 | HLA-DRB1 | 1.36E−15 | −0.408766643 | 0.847 | 0.941 | 3.13E−11 | −1.31E+10 |
| CD.Plsma/IgM.IGHM.JCHAIN | SARAF | 1.38E−15 | 0.347555128 | 0.749 | 0.573 | 3.17E−11 | 1.10E+10 |
| CD.Plsma/IgA.IGLL5.IGLV2-14 | RPS27A | 1.40E−15 | 0.3018991 | 1 | 0.981 | 3.21E−11 | 9.39E+09 |
| CD.Mac.APOE.PTGDS | RPL39 | 1.41E−15 | 0.255731716 | 0.889 | 0.914 | 3.25E−11 | 7.86E+09 |
| CD.Bclls.CD69.GPR183 | DYNLL1 | 1.45E−15 | 0.638031596 | 0.59 | 0.347 | 3.35E−11 | 1.91E+10 |
| CD.Plsma/IgM.IGHM.IGLV2-14 | RPS24 | 1.49E−15 | 0.522953482 | 0.876 | 0.827 | 3.43E−11 | 1.53E+10 |
| CD.Mono.S100A8.S100A9 | HSPA1B | 1.66E−15 | −1.600590612 | 0.267 | 0.663 | 3.81E−11 | −4.20E+10 |
| CD.Plsma/IgA.IGKV1-12.IGHA2 | RPL22 | 1.66E−15 | 0.778182055 | 0.902 | 0.661 | 3.82E−11 | 2.04E+10 |
| CD.Plsma/IgM.IGHM.IGLV2-14 | RPL5 | 1.67E−15 | 0.531212156 | 0.849 | 0.812 | 3.84E−11 | 1.38E+10 |
| CD.Plsma/IgA.AL9287683.RPL39 | MAP2K2 | 1.67E−15 | −0.323925461 | 0.159 | 0.346 | 3.85E−11 | −8.42E+09 |
| CD.B.RPS29.RPS21 | CD69 | 1.68E−15 | −0.499866922 | 0.572 | 0.727 | 3.86E−11 | −1.30E+10 |
| CD.Plsma/IgM.IGHM.JCHAIN | BTG1 | 1.69E−15 | 0.44852028 | 0.725 | 0.569 | 3.88E−11 | 1.16E+10 |
| CD.T.TRDC.TRGC2 | RPS26 | 1.77E−15 | 0.431544248 | 0.91 | 0.664 | 4.08E−11 | 1.06E+10 |
| CD.EC.MT1H.MT1G | PLA2G2A | 1.93E−15 | −1.498616877 | 0.043 | 0.569 | 4.45E−11 | −3.37E+10 |
| CD.Mac.APOE.PTGDS | CLEC12A | 1.95E−15 | −0.301631062 | 0.12 | 0.281 | 4.48E−11 | −6.73E+09 |
| CD.Fibro.TFPI2.CCL13 | IGFBP5 | 2.01E−15 | −1.283566173 | 0.68 | 0.849 | 4.63E−11 | −2.77E+10 |
| CD.B.IGHG1.IGLC2 | RPS10 | 2.07E−15 | 0.766044464 | 0.794 | 0.544 | 4.76E−11 | 1.61E+10 |
| CD.Plsma/IgA.IGKV1-12.IGHA2 | RPL14 | 2.14E−15 | 0.747792536 | 0.95 | 0.915 | 4.92E−11 | 1.52E+10 |
| CD.Plsma/IgA.IGHA2.IGKV1-12 | IGHV1-2 | 2.24E−15 | 0.938258159 | 0.235 | 0.044 | 5.16E−11 | 1.82E+10 |
| CD.Plsma/IgA.IGHA2.IGKV1-12 | RP11-16E12.2 | 2.42E−15 | −0.261201164 | 0.11 | 0.318 | 5.58E−11 | −4.68E+09 |
| CD.Fibro.TFPI2.CCL13 | RPS12 | 2.45E−15 | 0.39161602 | 0.99 | 0.978 | 5.64E−11 | 6.94E+09 |
| CD.B.MT-CO2.MT-CO1 | RPS20 | 2.48E−15 | 0.379746907 | 0.914 | 0.866 | 5.71E−11 | 6.65E+09 |
| CD.Bclls.CD69.GPR183 | RPL18A | 2.50E−15 | −0.266684513 | 0.989 | 0.991 | 5.76E−11 | −4.63E+09 |
| CD.Plsma/IgG.IGHGP.IGHG2 | RPS4Y1 | 2.50E−15 | 0.258206772 | 0.371 | 0.253 | 5.76E−11 | 4.48E+09 |
| CD.cDC1.CLEC9A.XCR1 | HLA-DRB1 | 2.58E−15 | −0.354063109 | 1 | 1 | 5.95E−11 | −5.95E+09 |
| CD.Plsma/IgA.IGKV1-12.IGHA2 | RPS12 | 2.60E−15 | 0.69231047 | 0.966 | 0.949 | 5.99E−11 | 1.16E+10 |
| CD.Plsma/IgA.IGHA1.IGLV6-57 | RPL9 | 2.61E−15 | 0.713774586 | 0.949 | 0.926 | 6.02E−11 | 1.19E+10 |
| CD.Mstcl.CTSG.CD69 | PABPC1 | 2.75E−15 | 0.749916996 | 0.725 | 0.439 | 6.34E−11 | 1.18E+10 |
| CD.Plsma/IgM.IGHM.JCHAIN | RPL23A | 2.87E−15 | 0.271553912 | 0.944 | 0.953 | 6.60E−11 | 4.11E+09 |
| CD.Paneth.DEFA6.ITLN2 | REG1B | 3.12E−15 | −1.018249916 | 0.566 | 0.819 | 7.17E−11 | −1.42E+10 |
| CD.Bclls.CD69.GPR183 | SERTAD1 | 3.19E−15 | 0.613191656 | 0.263 | 0.083 | 7.34E−11 | 8.36E+09 |
| CD.Fibro.TFPI2.CCL13 | EMILIN1 | 3.23E−15 | −0.83359965 | 0.406 | 0.774 | 7.42E−11 | −1.12E+10 |
| CD.Plsma/IgA.IGHA2.IGKV1-12 | UBA52 | 3.23E−15 | 0.344489523 | 0.888 | 0.791 | 7.44E−11 | 4.63E+09 |
| CD.Plsma/IgA.IGLV2-14.IGLV3-19 | XIST | 3.27E−15 | −0.584447439 | 0 | 0.338 | 7.53E−11 | −7.76E+09 |
| CD.B.MT-ND4L.IGHG1 | LUM | 3.35E−15 | 0.64678172 | 0.104 | 0.008 | 7.71E−11 | 8.38E+09 |
| CD.T.MT-CO2.MT-CO1 | MT-ND1 | 3.39E−15 | 0.595583744 | 0.908 | 0.753 | 7.81E−11 | 7.63E+09 |
| CD.Fibro.TFPI2.CCL13 | COL4A1 | 3.64E−15 | −0.636207184 | 0.098 | 0.452 | 8.37E−11 | −7.60E+09 |
| CD.Fibro.TFPI2.CCL13 | GSN | 3.69E−15 | −1.060184806 | 0.682 | 0.871 | 8.49E−11 | −1.25E+10 |
| CD.Plsma/IgA.IGLC3.IGLC2.2 | RPL12 | 4.02E−15 | −0.443408743 | 0.984 | 1 | 9.25E−11 | −4.80E+09 |
| CD.Plsma/IgG.IGHGP.IGHG2 | IGKV3-20 | 4.05E−15 | −0.511384518 | 0.164 | 0.278 | 9.32E−11 | −5.49E+09 |
| CD.Plsma/IgA.IGKV1-12.IGHA2 | MT-CYB | 4.16E−15 | 0.756791973 | 0.972 | 0.983 | 9.57E−11 | 7.91E+09 |
| CD.Plsma/IgM.IGHM.IGLV2-14 | RPL21 | 4.22E−15 | 0.332104426 | 0.96 | 0.974 | 9.70E−11 | 3.42E+09 |
| CD.Prolif/EC.UBE2C.CCNB2 | RPS26 | 4.37E−15 | 0.691199306 | 0.941 | 0.954 | 1.01E−10 | 6.87E+09 |
| CD.B.MT-CO2.MT-CO1 | SPINK4 | 4.40E−15 | 0.561407314 | 0.134 | 0.023 | 1.01E−10 | 5.54E+09 |
| CD.Plsma/IgA.IGLL5.IGLV2-14 | HSPA1A | 4.47E−15 | 1.08712734 | 0.757 | 0.519 | 1.03E−10 | 1.06E+10 |
| CD.Mstcl.CTSG.CD69 | MT-CO2 | 4.55E−15 | −0.725598416 | 0.947 | 0.962 | 1.05E−10 | −6.92E+09 |
| CD.Plsma/IgA.IGLL5.IGLV2-14 | NACA | 4.59E−15 | 0.410195496 | 0.967 | 0.867 | 1.06E−10 | 3.88E+09 |
| CD.EC.MTRNR2L1.MT-ND3 | JCHAIN | 4.65E−15 | 0.604011835 | 0.757 | 0.536 | 1.07E−10 | 5.65E+09 |
| CD.Plsma/IgA.IGLC3.IGLC2.2 | RPL27A | 4.84E−15 | −0.341584909 | 1 | 1 | 1.11E−10 | −3.07E+09 |
| CD.Fibro.TFPI2.CCL13 | CTNNB1 | 5.00E−15 | −0.8790437 | 0.114 | 0.473 | 1.15E−10 | −7.63E+09 |
| CD.Plsma/IgA.IGHA2.IGKV1-12 | SARAF | 5.09E−15 | 0.318734108 | 0.748 | 0.515 | 1.17E−10 | 2.72E+09 |
| CD.Bclls.CD69.GPR183 | HSPA8 | 5.09E−15 | 0.557939509 | 0.752 | 0.572 | 1.17E−10 | 4.76E+09 |
| CD.Plsma/IgG.IGHGP.IGHG2 | MT-ND2 | 5.17E−15 | 0.276292272 | 0.95 | 0.965 | 1.19E−10 | 2.32E+09 |
| CD.Plsma/IgM.IGHM.IGLV2-14 | MT-ND2 | 5.24E−15 | 0.448838149 | 0.96 | 0.981 | 1.21E−10 | 3.72E+09 |
| CD.Fibro.TFPI2.CCL13 | PTGER4 | 5.25E−15 | −0.59979123 | 0.119 | 0.495 | 1.21E−10 | −4.97E+09 |
| CD.B.MT-CO2.MT-CO1 | HSP90AA1 | 5.40E−15 | 0.683306252 | 0.77 | 0.61 | 1.24E−10 | 5.50E+09 |
| CD.T.MT-CO2.MT-CO3 | EEF1A1 | 5.50E−15 | 0.397030911 | 0.959 | 0.951 | 1.27E−10 | 3.13E+09 |
| CD.Plsma/IgM.IGHM.JCHAIN | RHOH | 5.60E−15 | 0.294202846 | 0.473 | 0.251 | 1.29E−10 | 2.28E+09 |
| CD.B.MT-CO2.MT-CO1 | RPS6 | 6.09E−15 | −0.278716292 | 0.967 | 0.987 | 1.40E−10 | −1.99E+09 |
| CD.Plsma/IgA.IGKV1-12.IGHA2 | IGHG3 | 6.21E−15 | −0.589442948 | 0.218 | 0.678 | 1.43E−10 | −4.12E+09 |
| CD.Mstcl.CTSG.CD69 | AC020571.3 | 6.29E−15 | 0.814888305 | 0.616 | 0.275 | 1.45E−10 | 5.63E+09 |
| CD.Plsma/IgM.IGHM.JCHAIN | RPL23 | 6.39E−15 | 0.361693062 | 0.853 | 0.745 | 1.47E−10 | 2.46E+09 |
| CD.T.KLRB1.LTF | RPL8 | 6.46E−15 | −0.281680681 | 0.947 | 0.952 | 1.49E−10 | −1.89E+09 |
| CD.Fibro.TFPI2.CCL13 | BDKRB1 | 6.61E−15 | −0.420997685 | 0.031 | 0.28 | 1.52E−10 | −2.77E+09 |
| CD.Plsma/IgA.AL9287683.RPL39 | RPL10A | 6.86E−15 | −0.258227108 | 0.976 | 0.987 | 1.58E−10 | −1.64E+09 |
| CD.EC.MT1H.MT1G | TMSB10 | 7.04E−15 | −1.207022243 | 0.483 | 0.889 | 1.62E−10 | −7.45E+09 |
| CD.Plsma/IgA.IGHA2.IGKV1-12 | RPS11 | 7.12E−15 | 0.366152801 | 0.941 | 0.889 | 1.64E−10 | 2.23E+09 |
| CD.Plsma/IgM.IGHM.IGLV2-14 | RPS13 | 7.16E−15 | 0.452319431 | 0.887 | 0.876 | 1.65E−10 | 2.74E+09 |
| CD.Plsma/IgM.IGHM.IGLV2-14 | MT-CYB | 7.23E−15 | 0.411750904 | 0.96 | 0.977 | 1.66E−10 | 2.48E+09 |
| CD.NK.CCL3.CD160 | PFN1 | 7.52E−15 | −0.257625736 | 0.903 | 0.915 | 1.73E−10 | −1.49E+09 |
| CD.Plsma/IgA.IGKV1-12.IGHA2 | MALAT1 | 7.53E−15 | 0.701886658 | 1 | 1 | 1.73E−10 | 4.05E+09 |
| CD.Plsma/IgG.IGHGP.IGHG2 | IGHV3-15 | 7.70E−15 | −0.819847851 | 0.081 | 0.175 | 1.77E−10 | −4.63E+09 |
| CD.Fibro.RERGL.NTRK2 | RPS26 | 7.86E−15 | 1.314925537 | 0.905 | 0.477 | 1.81E−10 | 7.26E+09 |
| CD.Mac.APOE.PTGDS | SH3BGRL3 | 8.15E−15 | −0.271794939 | 0.595 | 0.754 | 1.88E−10 | −1.45E+09 |
| CD.Bclls.CD69.GPR183 | LMNA | 8.22E−15 | 0.811238642 | 0.211 | 0.058 | 1.89E−10 | 4.29E+09 |
| CD.Plsma/IgA.IGLC3.IGLC2.2 | REG3A | 8.34E−15 | −0.369246428 | 0 | 0.28 | 1.92E−10 | −1.92E+09 |
| CD.TA.PLCG2.SOX4 | MT-ND2 | 8.55E−15 | 0.424057006 | 1 | 0.996 | 1.97E−10 | 2.16E+09 |
| CD.Mac.APOE.PTGDS | IGKC | 9.33E−15 | 0.649813562 | 0.966 | 0.955 | 2.15E−10 | 3.03E+09 |
| CD.Plsma/IgA.IGLV2-14.IGLV3-19 | RPL10 | 9.35E−15 | 0.607064183 | 1 | 0.985 | 2.15E−10 | 2.82E+09 |
| CD.Plsma/IgA.IGKV1-12.IGHA2 | RPL5 | 9.44E−15 | 0.679016129 | 0.961 | 0.881 | 2.17E−10 | 3.12E+09 |
| CD.Mac.APOE.PTGDS | RPS28 | 9.48E−15 | 0.279284835 | 0.888 | 0.897 | 2.18E−10 | 1.28E+09 |
| CD.cDC1.CLEC9A.XCR1 | DNAJB1 | 9.64E−15 | 0.678289854 | 0.867 | 0.692 | 2.22E−10 | 3.06E+09 |
| CD.NK.CCL3.CD160 | CACYBP | 9.89E−15 | 0.498886668 | 0.361 | 0.196 | 2.28E−10 | 2.19E+09 |
| CD.Plsma/IgA.IGHA2.IGKV1-12 | RPLP2 | 1.03E−14 | 0.283923773 | 0.962 | 0.963 | 2.37E−10 | 1.20E+09 |
| CD.Plsma/IgA.IGLC3.IGLC2.2 | MALAT1 | 1.06E−14 | −0.374970194 | 0.984 | 1 | 2.44E−10 | −1.53E+09 |
| CD.Fibro.TFPI2.CCL13 | SKIL | 1.13E−14 | −0.624732136 | 0.062 | 0.355 | 2.59E−10 | −2.41E+09 |
| CD.Bclls.CD69.GPR183 | REL | 1.14E−14 | 0.692744131 | 0.575 | 0.358 | 2.61E−10 | 2.65E+09 |
| CD.Plsma/IgM.IGHM.IGLV2-14 | RPS12 | 1.19E−14 | 0.398907559 | 0.925 | 0.891 | 2.75E−10 | 1.45E+09 |
| CD.Plsma/IgM.IGHM.JCHAIN | HSP90AA1 | 1.21E−14 | 0.706469294 | 0.924 | 0.88 | 2.77E−10 | 2.55E+09 |
| CD.Plsma/IgM.IGHM.IGLV2-14 | HNRNPA1 | 1.28E−14 | 0.565721402 | 0.736 | 0.538 | 2.94E−10 | 1.92E+09 |
| CD.Bclls.CD69.GPR183 | AC090498.1 | 1.29E−14 | −0.677550068 | 0.353 | 0.562 | 2.96E−10 | −2.29E+09 |
| CD.Plsma/IgA.IGHA2.IGKV1-12 | RPS15A | 1.31E−14 | 0.281397706 | 0.983 | 0.988 | 3.02E−10 | 9.31E+08 |
| CD.Mstcl.CTSG.CD69 | RPL39 | 1.35E−14 | 0.461363506 | 0.977 | 0.851 | 3.10E−10 | 1.49E+09 |
| CD.T.MT-CO2.MT-CO3 | AC090498.1 | 1.52E−14 | 0.817656188 | 0.248 | 0.049 | 3.50E−10 | 2.34E+09 |
| CD.Plsma/IgA.IGKV1-12.IGHA2 | HINT1 | 1.59E−14 | 0.69866958 | 0.885 | 0.61 | 3.66E−10 | 1.91E+09 |
| CD.Bclls.CD69.GPR183 | TSC22D3 | 1.63E−14 | 0.452922468 | 0.842 | 0.671 | 3.75E−10 | 1.21E+09 |
| CD.Mito.MT-CO2.MT-ND2 | CYBA | 1.69E−14 | −0.491261502 | 0.73 | 0.794 | 3.89E−10 | −1.26E+09 |
| CD.NK.CCL3.CD160 | RPS26 | 1.72E−14 | 0.333672944 | 0.731 | 0.541 | 3.95E−10 | 8.44E+08 |
| CD.NK.CCL3.CD160 | KLRF1 | 1.75E−14 | −0.48000976 | 0.472 | 0.593 | 4.03E−10 | −1.19E+09 |
| CD.NK.CCL3.CD160 | MRPL18 | 1.81E−14 | 0.52610897 | 0.229 | 0.093 | 4.17E−10 | 1.26E+09 |
| CD.Plsma/IgG.IGHGP.IGHG2 | UQCRB | 1.82E−14 | 0.251979475 | 0.73 | 0.646 | 4.20E−10 | 6.00E+08 |
| CD.NK.CCL3.CD160 | IL32 | 1.87E−14 | 0.377223591 | 0.508 | 0.318 | 4.30E−10 | 8.78E+08 |
| CD.Plsma/IgM.IGHM.JCHAIN | HINT1 | 1.87E−14 | 0.320794685 | 0.829 | 0.729 | 4.31E−10 | 7.44E+08 |
| CD.Plsma/IgA.MT-ND5.MT-ND4L | APOA1 | 1.88E−14 | 0.664809122 | 0.168 | 0 | 4.33E−10 | 1.53E+09 |
| CD.Plsma/IgA.IGHA1.IGLV6-57 | RPL21 | 1.98E−14 | 0.336450884 | 1 | 1 | 4.56E−10 | 7.37E+08 |
| CD.Plsma/IgA.IGHA1.IGKC | AL928768.3 | 2.04E−14 | 0.477217289 | 0.741 | 0.573 | 4.70E−10 | 1.01E+09 |
| CD.Plsma/IgA.AL9287683.RPL39 | NEAT1 | 2.07E−14 | −0.414275357 | 0.817 | 0.853 | 4.76E−10 | −8.69E+08 |
| CD.T.MT-CO2.MT-CO3 | IGHA2 | 2.08E−14 | 0.848437708 | 0.303 | 0.08 | 4.78E−10 | 1.78E+09 |
| CD.B.MT-CO2.MT-CO1 | RPL8 | 2.10E−14 | −0.342630048 | 0.904 | 0.961 | 4.83E−10 | −7.10E+08 |
| CD.Mac.APOE.PTGDS | CFP | 2.11E−14 | −0.348338924 | 0.114 | 0.26 | 4.85E−10 | −7.18E+08 |
| CD.Mono.S100A8.S100A9 | IGHA2 | 2.17E−14 | 0.902886348 | 0.438 | 0.128 | 4.99E−10 | 1.81E+09 |
| CD.Plsma/IgA.AL9287683.RPL39 | PABPC4 | 2.17E−14 | 0.258539514 | 0.728 | 0.529 | 5.00E−10 | 5.17E+08 |
| CD.Mac.APOE.PTGDS | CLEC9A | 2.20E−14 | 0.254871623 | 0.107 | 0.027 | 5.06E−10 | 5.04E+08 |
| CD.Plsma/IgM.IGHM.JCHAIN | AC090498.1 | 2.21E−14 | 0.316291019 | 0.517 | 0.308 | 5.10E−10 | 6.21E+08 |
| CD.Plsma/IgA.IGLL5.IGLV2-14 | RPS20 | 2.28E−14 | 0.321506565 | 0.975 | 0.932 | 5.24E−10 | 6.13E+08 |
| CD.Plsma/IgA.IGLC3.IGLC2.2 | HSPA1A | 2.28E−14 | 0.941305081 | 0.624 | 0.27 | 5.25E−10 | 1.79E+09 |
| CD.Fibro.TFPI2.CCL13 | RPL21 | 2.31E−14 | 0.281710943 | 0.997 | 0.989 | 5.31E−10 | 5.30E+08 |
| CD.NK.CCL3.CD160 | ARHGDIB | 2.46E−14 | 0.359719246 | 0.701 | 0.537 | 5.65E−10 | 6.36E+08 |
| CD.Mac.APOE.PTGDS | ACP5 | 2.52E−14 | −0.480368003 | 0.613 | 0.735 | 5.79E−10 | −8.29E+08 |
| CD.Plsma/IgA.IGLL5.IGLV2-14 | EEF1B2 | 2.65E−14 | 0.42363822 | 0.971 | 0.867 | 6.11E−10 | 6.94E+08 |
| CD.Fibro.TFPI2.CCL13 | VIM | 2.67E−14 | 0.566944698 | 0.974 | 0.925 | 6.16E−10 | 9.21E+08 |
| CD.Mito.MT-CO2.MT-ND2 | EEF1A1 | 2.72E−14 | 0.416518672 | 0.903 | 0.807 | 6.26E−10 | 6.66E+08 |
| CD.Plsma/IgA.IGLL5.IGLV2-14 | MT-CYB | 2.74E−14 | 0.366380195 | 0.967 | 0.973 | 6.31E−10 | 5.80E+08 |
| CD.T.CCL20.RORA | HLA-DRB5 | 2.88E−14 | 0.572856242 | 0.156 | 0.035 | 6.63E−10 | 8.64E+08 |
| CD.Fibro.TFPI2.CCL13 | MMP14 | 2.92E−14 | −0.424383906 | 0.034 | 0.28 | 6.72E−10 | −6.31E+08 |
| CD.T.CCL20.RORA | RPS4Y1 | 2.96E−14 | 0.625837612 | 0.297 | 0.113 | 6.80E−10 | 9.20E+08 |
| CD.cDC1.CLEC9A.XCR1 | EIF1 | 3.01E−14 | 0.309875773 | 0.994 | 1 | 6.92E−10 | 4.48E+08 |
| CD.Plsma/IgM.IGHM.IGLV2-14 | HSPA1A | 3.02E−14 | 0.816852949 | 0.771 | 0.594 | 6.96E−10 | 1.17E+09 |
| CD.Fibro.TFPI2.CCL13 | CYGB | 3.08E−14 | −0.918831852 | 0.398 | 0.753 | 7.08E−10 | −1.30E+09 |
| CD.Mac.APOE.PTGDS | FUOM | 3.10E−14 | −0.340357369 | 0.205 | 0.37 | 7.13E−10 | −4.78E+08 |
| CD.Prolif/EC.UBE2C.CCNB2 | JCHAIN | 3.18E−14 | 1.47507151 | 0.902 | 0.653 | 7.33E−10 | 2.01E+09 |
| CD.T.CCR7.SELL | TPT1 | 3.23E−14 | 0.364221541 | 0.989 | 0.97 | 7.45E−10 | 4.89E+08 |
| CD.B.RPS10.FCER2 | RPS4Y1 | 3.27E−14 | −0.948264788 | 0.152 | 0.527 | 7.54E−10 | −1.26E+09 |
| CD.Fibro.TFPI2.CCL13 | HGF | 3.51E−14 | −0.787303273 | 0.173 | 0.538 | 8.09E−10 | −9.73E+08 |
| CD.Mono.CXCL10.TNF | CCL4L2 | 3.53E−14 | 0.758433175 | 0.97 | 0.902 | 8.12E−10 | 9.34E+08 |
| CD.T.CCR7.GPR183 | RPS10 | 3.80E−14 | 0.315778737 | 0.653 | 0.489 | 8.75E−10 | 3.61E+08 |
| CD.Plsma/IgM.IGHM.JCHAIN | RPS4Y1 | 4.43E−14 | 0.314519795 | 0.36 | 0.176 | 1.02E−09 | 3.08E+08 |
| CD.TA.PLCG2.SOX4 | RPS2 | 4.51E−14 | −0.652887525 | 0.872 | 0.932 | 1.04E−09 | −6.29E+08 |
| CD.NK.CCL3.CD160 | MIF | 4.59E−14 | 0.444423442 | 0.338 | 0.177 | 1.06E−09 | 4.20E+08 |
| CD.Endth/Cap.CD36.FLT1 | XIST | 4.84E−14 | −1.11536962 | 0.084 | 0.371 | 1.11E−09 | −1.00E+09 |
| CD.Fibro.TFPI2.CCL13 | TUBA1A | 4.97E−14 | 0.998941309 | 0.76 | 0.505 | 1.14E−09 | 8.73E+08 |
| CD.Fibro.TFPI2.CCL13 | SNHG9 | 4.98E−14 | −0.514956995 | 0.054 | 0.333 | 1.15E−09 | −4.49E+08 |
| CD.Mac.APOE.PTGDS | MT-ND3 | 5.65E−14 | 0.419805572 | 0.771 | 0.805 | 1.30E−09 | 3.23E+08 |
| CD.Plsma/IgA.IGHA1.IGLV6-57 | RPS24 | 5.78E−14 | 0.690753699 | 0.932 | 0.907 | 1.33E−09 | 5.19E+08 |
| CD.Plsma/IgA.IGKV1-12.IGHA2 | IGHG4 | 5.84E−14 | −0.543118218 | 0.342 | 0.814 | 1.35E−09 | −4.04E+08 |
| CD.Prolif/EC.UBE2C.CCNB2 | MIF | 5.87E−14 | 1.111598376 | 0.843 | 0.575 | 1.35E−09 | 8.23E+08 |
| CD.Plsma/IgA.IGLV2-14.IGLV3-19 | RPS24 | 5.88E−14 | 0.711987162 | 0.97 | 0.794 | 1.35E−09 | 5.26E+08 |
| CD.Plsma/IgA.IGHA2.IGKV1-12 | HNRNPA1 | 5.92E−14 | 0.333425815 | 0.778 | 0.586 | 1.36E−09 | 2.45E+08 |
| CD.Fibro.TFPI2.CCL13 | PABPC1 | 6.00E−14 | −0.765049214 | 0.333 | 0.742 | 1.38E−09 | −5.54E+08 |
| CD.Plsma/IgA.IGHA2.IGKV1-12 | CCR10 | 6.00E−14 | 0.299919945 | 0.472 | 0.224 | 1.38E−09 | 2.17E+08 |
| CD.T.CCL20.RORA | RPS26 | 6.17E−14 | 0.40831389 | 0.878 | 0.655 | 1.42E−09 | 2.87E+08 |
| CD.Plsma/IgA.IGHA2.IGKV1-12 | TNFRSF13B | 6.24E−14 | 0.258675231 | 0.303 | 0.099 | 1.44E−09 | 1.80E+08 |
| CD.Bclls.CD69.GPR183 | MT-ATP6 | 6.49E−14 | 0.376829271 | 0.944 | 0.882 | 1.49E−09 | 2.52E+08 |
| CD.B.ACTB.JCHAIN | IGHA1 | 6.85E−14 | 0.832306167 | 0.916 | 0.754 | 1.58E−09 | 5.28E+08 |
| CD.T.KLRB1.LTF | IGLC2 | 6.88E−14 | −0.530412462 | 0.361 | 0.569 | 1.58E−09 | −3.35E+08 |
| CD.T.KLRB1.LTF | EIF5A | 7.03E−14 | −0.44333877 | 0.212 | 0.417 | 1.62E−09 | −2.74E+08 |
| CD.Plsma/IgA.IGKV1-12.IGHA2 | RPS5 | 7.13E−14 | 0.606127279 | 0.978 | 0.949 | 1.64E−09 | 3.69E+08 |
| CD.Plsma/IgM.IGHM.IGLV2-14 | RPLP2 | 7.42E−14 | 0.400745622 | 0.922 | 0.929 | 1.71E−09 | 2.35E+08 |
| CD.cDC1.CLEC9A.XCR1 | EEF1A1 | 7.51E−14 | 0.333828362 | 1 | 1 | 1.73E−09 | 1.93E+08 |
| CD.Plsma/IgA.IGHA2.IGKV1-12 | ATP5E | 7.80E−14 | −0.284881605 | 0.788 | 0.872 | 1.80E−09 | −1.59E+08 |
| CD.Plsma/IgA.IGLL5.IGLV2-14 | RPL10 | 7.98E−14 | 0.260571589 | 1 | 0.996 | 1.84E−09 | 1.42E+08 |
| CD.NK.GNLY.KLRC1 | IGLC2 | 8.05E−14 | 1.213126045 | 0.8 | 0.63 | 1.85E−09 | 6.54E+08 |
| CD.Plsma/IgM.IGHM.IGLV2-14 | VIM | 8.06E−14 | 0.602574128 | 0.792 | 0.662 | 1.85E−09 | 3.25E+08 |
| CD.T.CARD16.GB2 | MT-ND3 | 8.10E−14 | 0.384763832 | 0.87 | 0.698 | 1.86E−09 | 2.06E+08 |
| CD.Plsma/IgA.IGHA2.IGKV1-12 | RPS21 | 8.23E−14 | −0.316473699 | 0.502 | 0.717 | 1.89E−09 | −1.67E+08 |
| CD.Fibro.TFPI2.CCL13 | QSOX1 | 8.31E−14 | −0.778812331 | 0.556 | 0.839 | 1.91E−09 | −4.07E+08 |
| CD.T.KLRB1.LTF | MT-CYB | 8.33E−14 | 0.27932479 | 0.948 | 0.903 | 1.92E−09 | 1.46E+08 |
| CD.T.MT-CO2.MT-CO1 | JCHAIN | 8.58E−14 | 0.705685886 | 0.703 | 0.435 | 1.98E−09 | 3.57E+08 |
| CD.B.IGHG1.IGLC2 | RPL39 | 8.62E−14 | 0.298797219 | 0.999 | 0.992 | 1.98E−09 | 1.51E+08 |
| CD.Mac.APOE.PTGDS | CXCL12 | 8.83E−14 | 0.465772988 | 0.283 | 0.148 | 2.03E−09 | 2.29E+08 |
| CD.Fibro.TFPI2.CCL13 | S100A6 | 9.01E−14 | −0.602053688 | 0.959 | 0.968 | 2.07E−09 | −2.90E+08 |
| CD.Plsma/IgA.IGKV1-12.IGHA2 | EEF1B2 | 9.02E−14 | 0.769959775 | 0.936 | 0.831 | 2.08E−09 | 3.71E+08 |
| CD.Plsma/IgA.IGLC3.IGLC2.2 | PABPC4 | 9.15E−14 | 0.516423142 | 0.78 | 0.5 | 2.11E−09 | 2.45E+08 |
| CD.EC.MTRNR2L1.MT-ND3 | APOA1 | 9.30E−14 | 0.704107833 | 0.146 | 0.003 | 2.14E−09 | 3.29E+08 |
| CD.cDC1.CLEC9A.XCR1 | MT-ND3 | 9.48E−14 | 0.441431628 | 0.988 | 0.924 | 2.18E−09 | 2.02E+08 |
| CD.EC.MT1H.MT1G | DEFA5 | 9.73E−14 | −1.322016537 | 0.103 | 0.653 | 2.24E−09 | −5.90E+08 |
| CD.B.MT-CO2.MT-CO1 | PFN1 | 9.81E−14 | −0.537422331 | 0.565 | 0.755 | 2.26E−09 | −2.38E+08 |
| CD.B.IGLC3.JCHAIN | RPS29 | 9.91E−14 | 0.541614316 | 0.979 | 1 | 2.28E−09 | 2.37E+08 |
| CD.Plsma/IgM.IGHM.IGLV2-14 | MTRNR2L8 | 9.97E−14 | 0.811027906 | 0.375 | 0.124 | 2.30E−09 | 3.53E+08 |
| CD.T.MT-CO2.MT-CO3 | PTPRC | 1.01E−13 | −0.622668732 | 0.592 | 0.754 | 2.33E−09 | −2.67E+08 |
| CD.Plsma/IgA.IGHA2.IGKV1-12 | UQCRB | 1.02E−13 | 0.367162062 | 0.773 | 0.594 | 2.34E−09 | 1.57E+08 |
| CD.NK.CCL3.CD160 | CCL3 | 1.03E−13 | −0.575130121 | 0.416 | 0.591 | 2.37E−09 | −2.43E+08 |
| CD.Plsma/IgA.IGLL5.IGLV2-14 | RPS3 | 1.05E−13 | 0.315308072 | 1 | 0.977 | 2.42E−09 | 1.30E+08 |
| CD.Mono.S100A8.S100A9 | IGHG1 | 1.07E−13 | 0.907259641 | 0.581 | 0.252 | 2.47E−09 | 3.68E+08 |
| CD.Plsma/IgA.IGLC3.IGLC2.2 | MZB1 | 1.10E−13 | −0.33043908 | 0.995 | 1 | 2.54E−09 | −1.30E+08 |
| CD.Plsma/IgA.IGLC3.IGLC2.1 | JSRP1 | 1.12E−13 | 0.372513258 | 0.453 | 0.185 | 2.57E−09 | 1.45E+08 |
| CD.Plsma/IgM.IGHM.IGLV2-14 | H3F3B | 1.14E−13 | 0.510712007 | 0.925 | 0.883 | 2.63E−09 | 1.94E+08 |
| CD.Bclls.CD69.GPR183 | CWC25 | 1.15E−13 | 0.474220437 | 0.229 | 0.069 | 2.64E−09 | 1.79E+08 |
| CD.Bclls.CD69.GPR183 | DNAJB1 | 1.15E−13 | 0.624975997 | 0.549 | 0.307 | 2.65E−09 | 2.36E+08 |
| CD.T.TRDC.TRGC2 | RPL37A | 1.16E−13 | 0.383994939 | 0.897 | 0.745 | 2.67E−09 | 1.44E+08 |
| CD.Mono.CXCL10.TNF | RPS10 | 1.19E−13 | 0.778753641 | 0.799 | 0.66 | 2.75E−09 | 2.84E+08 |
| CD.Plsma/IgA.HSPA1B.HSPA1A | CD48 | 1.20E−13 | 0.406600349 | 0.472 | 0.139 | 2.76E−09 | 1.48E+08 |
| CD.Plsma/IgA.IGHA2.IGKV1-12 | GLTSCR2 | 1.21E−13 | 0.336567138 | 0.697 | 0.485 | 2.77E−09 | 1.21E+08 |
| CD.Plsma/IgA.IGKV1-12.IGHA2 | SPCS1 | 1.21E−13 | 0.689619427 | 0.927 | 0.814 | 2.78E−09 | 2.48E+08 |
| CD.Plsma/IgM.IGHM.JCHAIN | BTG2 | 1.24E−13 | −0.276672069 | 0.777 | 0.863 | 2.86E−09 | −9.67E+07 |
| CD.Fibro.TFPI2.CCL13 | CBX3 | 1.32E−13 | −0.577364542 | 0.142 | 0.516 | 3.03E−09 | −1.91E+08 |
| CD.Plsma/IgA.IGLV2-14.IGLV3-19 | IGLV3-19 | 1.32E−13 | 2.103980368 | 0.575 | 0.029 | 3.03E−09 | 6.95E+08 |
| CD.Fibro.TFPI2.CCL13 | CDH11 | 1.35E−13 | −0.727487932 | 0.194 | 0.581 | 3.11E−09 | −2.34E+08 |
| CD.Mito.MT-CO2.MT-ND2 | B2M | 1.38E−13 | −0.330025858 | 0.984 | 0.982 | 3.18E−09 | −1.04E+08 |
| CD.B.RPS29.RPS21 | AC079767.4 | 1.40E−13 | −0.529183377 | 0.102 | 0.235 | 3.22E−09 | −1.65E+08 |
| CD.TA.PLCG2.SOX4 | CD74 | 1.46E−13 | −0.843000491 | 0.474 | 0.771 | 3.37E−09 | −2.50E+08 |
| CD.Mono.CXCL10.TNF | EIF5A | 1.51E−13 | −0.611987712 | 0.213 | 0.667 | 3.47E−09 | −1.76E+08 |
| CD.Endth/Cap.CD36.FLT1 | IGHA1 | 1.51E−13 | 0.450053205 | 0.979 | 0.815 | 3.48E−09 | 1.29E+08 |
| CD.Mstcl.CTSG.CD69 | RPL41 | 1.52E−13 | 0.392225937 | 0.967 | 0.821 | 3.50E−09 | 1.12E+08 |
| CD.Plsma/IgA.HSPA1B.HSPA1A | RPL9 | 1.53E−13 | 0.368226638 | 0.944 | 0.993 | 3.52E−09 | 1.05E+08 |
| CD.T.CCL20.RORA | XIST | 1.58E−13 | −0.973278813 | 0.091 | 0.304 | 3.64E−09 | −2.67E+08 |
| CD.NK.CCL3.CD160 | SH2D1A | 1.58E−13 | −0.50560203 | 0.284 | 0.426 | 3.65E−09 | −1.39E+08 |
| CD.B.RPS29.RPS21 | ZFP36 | 1.61E−13 | 0.444280322 | 0.442 | 0.27 | 3.71E−09 | 1.20E+08 |
| CD.NK.CCL3.CD160 | RPS20 | 1.64E−13 | 0.270264253 | 0.85 | 0.732 | 3.79E−09 | 7.14E+07 |
| CD.Bclls.CD69.GPR183 | ZFAS1 | 1.67E−13 | 0.575818668 | 0.647 | 0.435 | 3.84E−09 | 1.50E+08 |
| CD.Plsma/IgA.IGKV1-12.IGHA2 | VIM | 1.68E−13 | 0.999954995 | 0.849 | 0.576 | 3.86E−09 | 2.59E+08 |
| CD.Paneth.DEFA6.ITLN2 | GOLGB1 | 1.71E−13 | 0.444967223 | 0.532 | 0.185 | 3.94E−09 | 1.13E+08 |
| CD.Plsma/IgA.IGHA2.IGKV1-12 | HSP90AA1 | 1.72E−13 | 0.595008696 | 0.903 | 0.842 | 3.96E−09 | 1.50E+08 |
| CD.Bclls.CD69.GPR183 | TRA2B | 1.77E−13 | 0.593611619 | 0.425 | 0.209 | 4.07E−09 | 1.46E+08 |
| CD.B.MT-CO2.MT-CO1 | XIST | 1.78E−13 | −0.96090075 | 0.043 | 0.279 | 4.10E−09 | −2.34E+08 |
| CD.NK.CCL3.CD160 | GZMH | 1.85E−13 | −0.664673354 | 0.084 | 0.202 | 4.26E−09 | −1.56E+08 |
| CD.Plsma/IgA.HSPA1B.HSPA1A | RPS23 | 1.93E−13 | 0.420588237 | 0.967 | 0.974 | 4.43E−09 | 9.49E+07 |
| CD.Fibro.TFPI2.CCL13 | LUM | 1.95E−13 | −0.477504311 | 0.995 | 0.978 | 4.48E−09 | −1.07E+08 |
| CD.T.MT-CO2.MT-CO3 | MALAT1 | 1.95E−13 | 0.313814683 | 1 | 0.998 | 4.49E−09 | 6.99E+07 |
| CD.Plsma/IgA.HSPA1B.HSPA1A | BTG2 | 1.97E−13 | −0.423068759 | 0.86 | 0.96 | 4.54E−09 | −9.32E+07 |
| CD.Plsma/IgA.IGLC3.IGLC2.2 | RPL19 | 2.01E−13 | −0.340953249 | 0.995 | 1 | 4.63E−09 | −7.37E+07 |
| CD.Plsma/IgA.IGHA1.IGLV6-57 | XIST | 2.02E−13 | −0.751920993 | 0.162 | 0.554 | 4.64E−09 | −1.62E+08 |
| CD.Plsma/IgM.IGHM.JCHAIN | DERL3 | 2.21E−13 | −0.287567722 | 0.837 | 0.908 | 5.09E−09 | −5.65E+07 |
| CD.Fibro.TFPI2.CCL13 | PGRMC2 | 2.22E−13 | −0.610852418 | 0.199 | 0.591 | 5.10E−09 | −1.20E+08 |
| CD.Bclls.CD69.GPR183 | ZFP36 | 2.36E−13 | 0.579610575 | 0.617 | 0.361 | 5.42E−09 | 1.07E+08 |
| CD.Plsma/IgA.IGHA2.IGKV1-12 | EIF1 | 2.36E−13 | 0.271371524 | 0.968 | 0.978 | 5.43E−09 | 4.99E+07 |
| CD.Plsma/IgM.IGHM.JCHAIN | PABPC4 | 2.41E−13 | 0.354600974 | 0.557 | 0.378 | 5.54E−09 | 6.40E+07 |
| CD.Plsma/IgA.AL9287683.RPL39 | MT2A | 2.45E−13 | −0.520230566 | 0.388 | 0.533 | 5.63E−09 | −9.24E+07 |
| CD.T.CD8B.TRGC2 | KLRC1 | 2.46E−13 | −0.635527735 | 0.1 | 0.249 | 5.67E−09 | −1.12E+08 |
| CD.Mito.MT-CO2.MT-ND2 | IGHA2 | 2.48E−13 | 0.46283874 | 0.397 | 0.189 | 5.71E−09 | 8.10E+07 |
| CD.Mito.MT-CO2.MT-ND2 | TPT1 | 2.64E−13 | 0.572198229 | 0.839 | 0.728 | 6.09E−09 | 9.40E+07 |
| CD.Plsma/IgA.MT-ND5.MT-ND4L | FABP6 | 2.72E−13 | 0.466110029 | 0.154 | 0 | 6.26E−09 | 7.44E+07 |
| CD.Plsma/IgA.IGHA1.IGLV6-57 | MT-ND4L | 2.76E−13 | −0.670372165 | 0.291 | 0.659 | 6.34E−09 | −1.06E+08 |
| CD.Mac.APOE.PTGDS | DNAJC15 | 2.90E−13 | −0.262424799 | 0.228 | 0.386 | 6.68E−09 | −3.93E+07 |
| CD.Fibro.TFPI2.CCL13 | B4GALT1 | 3.04E−13 | −0.576380088 | 0.047 | 0.301 | 6.99E−09 | −8.24E+07 |
| CD.Fibro.TFPI2.CCL13 | EEF1B2 | 3.20E−13 | 0.679899593 | 0.855 | 0.742 | 7.37E−09 | 9.23E+07 |
| CD.Paneth.DEFA6.ITLN2 | RND3 | 3.20E−13 | 0.459271541 | 0.254 | 0.024 | 7.38E−09 | 6.23E+07 |
| CD.Plsma/IgA.IGLV2-14.IGLV3-19 | PRDX4 | 3.23E−13 | 0.642773869 | 0.97 | 0.75 | 7.45E−09 | 8.63E+07 |
| CD.Plsma/IgA.IGLC3.IGLC2.2 | HLA-A | 3.34E−13 | −0.404364238 | 0.968 | 0.985 | 7.70E−09 | −5.25E+07 |
| CD.Plsma/IgG.IGHGP.IGHG2 | IGKV3-11 | 3.37E−13 | −0.434395891 | 0.116 | 0.212 | 7.76E−09 | −5.60E+07 |
| CD.Plsma/IgA.IGKV1-12.IGHA2 | SEC11C | 3.43E−13 | 0.660882365 | 0.938 | 0.864 | 7.90E−09 | 8.37E+07 |
| CD.Paneth.DEFA6.ITLN2 | FABP1 | 3.51E−13 | 0.417918611 | 0.327 | 0.059 | 8.09E−09 | 5.17E+07 |
| CD.Plsma/IgA.HSPA1B.HSPA1A | EEF1B2 | 3.61E−13 | 0.513221875 | 0.909 | 0.848 | 8.30E−09 | 6.18E+07 |
| CD.Bclls.CD69.GPR183 | RPL36 | 3.70E−13 | −0.275098103 | 0.959 | 0.973 | 8.52E−09 | −3.23E+07 |
| CD.NK.CCL3.CD160 | TUBA1A | 4.04E−13 | 0.414276002 | 0.345 | 0.184 | 9.30E−09 | 4.46E+07 |
| CD.Bclls.CD69.GPR183 | HSPD1 | 4.32E−13 | 0.673710191 | 0.508 | 0.306 | 9.94E−09 | 6.78E+07 |
| CD.Prolif/EC.UBE2C.CCNB2 | NME2 | 4.34E−13 | 0.290911397 | 0.255 | 0.028 | 9.99E−09 | 2.91E+07 |
| CD.EC.MT1H.MT1G | PPDPF | 4.42E−13 | −1.216254443 | 0.078 | 0.583 | 1.02E−08 | −1.19E+08 |
| CD.Fibro.TFPI2.CCL13 | EIF1 | 4.56E−13 | 0.519089049 | 0.982 | 0.882 | 1.05E−08 | 4.94E+07 |
| CD.Bclls.CD69.GPR183 | VPREB3 | 4.64E−13 | −0.547364689 | 0.515 | 0.694 | 1.07E−08 | −5.12E+07 |
| CD.Plsma/IgA.IGKV1-12.IGHA2 | HSPA1A | 4.71E−13 | 1.374081317 | 0.838 | 0.542 | 1.08E−08 | 1.27E+08 |
| CD.cDC1.CLEC9A.XCR1 | RPL30 | 4.76E−13 | 0.35857035 | 0.994 | 1 | 1.09E−08 | 3.28E+07 |
| CD.B.MT-CO2.MT-CO1 | MT-CO3 | 4.93E−13 | 0.359113115 | 0.99 | 0.994 | 1.14E−08 | 3.16E+07 |
| CD.Mstcl.CTSG.CD69 | IL1RL1 | 4.99E−13 | −1.08614576 | 0.192 | 0.45 | 1.15E−08 | −9.45E+07 |
| CD.NK.CCL3.CD160 | NACA | 5.07E−13 | 0.370412852 | 0.667 | 0.534 | 1.17E−08 | 3.18E+07 |
| CD.T.CCL4.GMZK | RPS4Y1 | 5.12E−13 | −0.507377764 | 0.135 | 0.287 | 1.18E−08 | −4.30E+07 |
| CD.Fibro.TFPI2.CCL13 | ALDH1A3 | 5.16E−13 | −0.656776477 | 0.124 | 0.452 | 1.19E−08 | −5.53E+07 |
| CD.Plsma/IgA.IGKV1-12.IGHA2 | RPL7 | 5.24E−13 | 0.563355125 | 0.978 | 0.966 | 1.21E−08 | 4.67E+07 |
| CD.Mono.CXCL10.TNF | PABPC1 | 5.27E−13 | −0.657558612 | 0.732 | 0.902 | 1.21E−08 | −5.42E+07 |
| CD.Plsma/IgA.AL9287683.RPL39 | NUCB2 | 5.50E−13 | −0.35444776 | 0.586 | 0.68 | 1.27E−08 | −2.80E+07 |
| CD.Plsma/IgA.MT-ND5.MT-ND4L | MT-CO3 | 5.78E−13 | 0.642813491 | 1 | 0.978 | 1.33E−08 | 4.83E+07 |
| CD.Plsma/IgA.IGLL5.IGLV2-14 | MT-ATP6 | 5.81E−13 | 0.421392306 | 0.962 | 0.894 | 1.34E−08 | 3.15E+07 |
| CD.Plsma/IgA.MT-ND5.MT-ND4L | MT-ATP6 | 6.25E−13 | 0.705676208 | 0.972 | 0.916 | 1.44E−08 | 4.91E+07 |
| CD.Plsma/IgA.IGKV1-12.IGHA2 | RPL29 | 6.27E−13 | 0.588467515 | 0.947 | 0.915 | 1.44E−08 | 4.08E+07 |
| CD.Fibro.TFPI2.CCL13 | RNF13 | 6.50E−13 | −0.609510441 | 0.132 | 0.484 | 1.50E−08 | −4.08E+07 |
| CD.Mono.CXCL3.FCN1 | IGHG1 | 6.50E−13 | 1.27912235 | 0.716 | 0.315 | 1.50E−08 | 8.55E+07 |
| CD.Plsma/IgA.IGLC3.IGLC2.2 | RPLP1 | 6.58E−13 | −0.259570773 | 0.995 | 1 | 1.51E−08 | −1.71E+07 |
| CD.cDC1.CLEC9A.XCR1 | CRIP1 | 6.79E−13 | 0.478370424 | 0.566 | 0.207 | 1.56E−08 | 3.06E+07 |
| CD.T.CCR7.SELL | KLF2 | 6.83E−13 | −0.614199816 | 0.165 | 0.269 | 1.57E−08 | −3.91E+07 |
| CD.cDC1.CLEC9A.XCR1 | RPS20 | 6.94E−13 | 0.329221163 | 0.983 | 1 | 1.60E−08 | 2.06E+07 |
| CD.Fibro.TFPI2.CCL13 | C11orf96 | 6.96E−13 | −0.713582803 | 0.227 | 0.591 | 1.60E−08 | −4.45E+07 |
| CD.Mono.S100A8.S100A9 | SPINK4 | 7.00E−13 | 0.714510866 | 0.21 | 0.027 | 1.61E−08 | 4.44E+07 |
| CD.Plsma/IgA.IGLV2-14.IGLV3-19 | RPL13 | 7.04E−13 | 0.505351738 | 1 | 1 | 1.62E−08 | 3.12E+07 |
| CD.NK.GNLY.KLRC1 | RPS10 | 7.30E−13 | 0.866042682 | 0.786 | 0.6 | 1.68E−08 | 5.16E+07 |
| CD.Bclls.CD69.GPR183 | HSPH1 | 7.31E−13 | 0.665698197 | 0.447 | 0.236 | 1.68E−08 | 3.95E+07 |
| CD.Fibro.TFPI2.CCL13 | PET100 | 7.43E−13 | −0.849678878 | 0.14 | 0.484 | 1.71E−08 | −4.97E+07 |
| CD.Plsma/IgA.IGKV1-12.IGHA2 | SOCS3 | 7.58E−13 | −0.614515189 | 0.451 | 0.864 | 1.74E−08 | −3.52E+07 |
| CD.Plsma/IgA.IGLV2-14.IGLV3-19 | RPL5 | 7.60E−13 | 0.656396372 | 0.97 | 0.838 | 1.75E−08 | 3.75E+07 |
| CD.Plsma/IgA.IGHA2.IGKV1-12 | DNAJB9 | 7.70E−13 | −0.323532019 | 0.72 | 0.818 | 1.77E−08 | −1.82E+07 |
| CD.T.CCL4.GMZK | H3F3B | 7.72E−13 | 0.281299651 | 0.92 | 0.849 | 1.78E−08 | 1.58E+07 |
| CD.Plsma/IgA.IGLV2-14.IGLV3-19 | RPS29 | 7.74E−13 | −0.646630875 | 0.892 | 0.985 | 1.78E−08 | −3.63E+07 |
| CD.Mito.MT-CO2.MT-ND2 | TMSB10 | 7.80E−13 | −0.361925499 | 0.845 | 0.924 | 1.80E−08 | −2.02E+07 |
| CD.EC.OLFM4.MT-ND2 | REG1B | 7.88E−13 | −1.682409051 | 0.158 | 0.756 | 1.81E−08 | −9.27E+07 |
| CD.NK.CCL3.CD160 | ARF6 | 7.94E−13 | −0.504444125 | 0.303 | 0.435 | 1.83E−08 | −2.76E+07 |
| CD.Plsma/IgA.IGLV2-14.IGLV3-19 | RPL13A | 8.48E−13 | 0.55732677 | 1 | 1 | 1.95E−08 | 2.86E+07 |
| CD.Prolif/EC.UBE2C.CCNB2 | FTH1 | 8.60E−13 | 0.428081364 | 1 | 0.992 | 1.98E−08 | 2.16E+07 |
| CD.Plsma/IgA.IGLL5.IGLV2-14 | HSPB1 | 8.85E−13 | 0.49725187 | 0.556 | 0.235 | 2.04E−08 | 2.44E+07 |
| CD.TA.PLCG2.SOX4 | TMSB10 | 8.85E−13 | −0.540923093 | 0.84 | 0.97 | 2.04E−08 | −2.66E+07 |
| CD.Fibro.TFPI2.CCL13 | MEG3 | 8.97E−13 | −0.659506257 | 0.284 | 0.688 | 2.06E−08 | −3.20E+07 |
| CD.Mac.APOE.PTGDS | CD72 | 9.68E−13 | −0.262332377 | 0.127 | 0.267 | 2.23E−08 | −1.18E+07 |
| CD.T.CCL4.GMZK | CD8B | 1.01E−12 | 0.489114296 | 0.26 | 0.111 | 2.33E−08 | 2.10E+07 |
| CD.Mac.APOE.PTGDS | RPS26 | 1.07E−12 | 0.428897685 | 0.693 | 0.672 | 2.46E−08 | 1.74E+07 |
| CD.Plsma/IgG.IGHGP.IGHG2 | HIST1H2AC | 1.08E−12 | 0.282375927 | 0.277 | 0.181 | 2.48E−08 | 1.14E+07 |
| CD.Goblet.FCGBP.SPINK4 | MTRNR2L8 | 1.10E−12 | 1.407305227 | 0.795 | 0.3 | 2.53E−08 | 5.55E+07 |
| CD.Mac.APOE.PTGDS | CYCS | 1.12E−12 | −0.272600086 | 0.42 | 0.597 | 2.57E−08 | −1.06E+07 |
| CD.Bclls.CD69.GPR183 | CD69 | 1.12E−12 | 0.606742174 | 0.921 | 0.835 | 2.58E−08 | 2.35E+07 |
| CD.Plsma/IgM.IGHM.IGLV2-14 | MT-ND3 | 1.13E−12 | 0.391370795 | 0.933 | 0.947 | 2.59E−08 | 1.51E+07 |
| CD.T.CARD16.GB2 | MT-ND4L | 1.17E−12 | −1.007162166 | 0.081 | 0.269 | 2.70E−08 | −3.74E+07 |
| CD.T.MT-CO2.MT-CO3 | RPL30 | 1.19E−12 | 0.457977166 | 0.844 | 0.749 | 2.74E−08 | 1.67E+07 |
| CD.Plsma/IgM.IGHM.JCHAIN | SRGN | 1.24E−12 | 0.302503671 | 0.857 | 0.835 | 2.85E−08 | 1.06E+07 |
| CD.EC.MT1H.MT1G | REG1B | 1.29E−12 | −1.882932757 | 0.026 | 0.431 | 2.97E−08 | −6.34E+07 |
| CD.T.CCL20.RORA | EEF1A1 | 1.30E−12 | 0.270147847 | 0.981 | 0.984 | 2.98E−08 | 9.05E+06 |
| CD.Mono.S100A8.S100A9 | RPS24 | 1.30E−12 | 0.503653052 | 0.952 | 0.905 | 2.99E−08 | 1.68E+07 |
| CD.Plsma/IgA.IGHA1.IGLV6-57 | RPL30 | 1.31E−12 | 0.654128222 | 0.923 | 0.92 | 3.01E−08 | 2.17E+07 |
| CD.Plsma/IgA.IGLC3.IGLC2.1 | UBC | 1.31E−12 | −0.420150938 | 0.9 | 0.956 | 3.01E−08 | −1.40E+07 |
| CD.Bclls.CD69.GPR183 | IGLC3 | 1.33E−12 | −0.558558794 | 0.432 | 0.623 | 3.05E−08 | −1.83E+07 |
| CD.Plsma/IgA.IGHA2.IGKV1-12 | JUNB | 1.35E−12 | −0.343266195 | 0.824 | 0.906 | 3.11E−08 | −1.10E+07 |
| CD.B.RPS29.RPS21 | CD83 | 1.39E−12 | −0.364705407 | 0.541 | 0.66 | 3.19E−08 | −1.14E+07 |
| CD.Plsma/IgA.HSPA1B.HSPA1A | C8orf59 | 1.40E−12 | 0.422351663 | 0.49 | 0.205 | 3.22E−08 | 1.31E+07 |
| CD.Paneth.DEFA6.ITLN2 | RPS2 | 1.41E−12 | −0.430383809 | 0.976 | 0.949 | 3.25E−08 | −1.32E+07 |
| CD.Fibro.TFPI2.CCL13 | H3F3B | 1.42E−12 | 0.42704156 | 0.969 | 0.914 | 3.27E−08 | 1.31E+07 |
| CD.Plsma/IgA.IGLC3.IGLC2.2 | RPS9 | 1.43E−12 | −0.340397591 | 1 | 1 | 3.28E−08 | −1.04E+07 |
| CD.Plsma/IgA.IGHA1.IGKC | EEF1A1 | 1.47E−12 | 0.314820691 | 0.971 | 0.979 | 3.37E−08 | 9.33E+06 |
| CD.B.MT-CO2.MT-CO1 | RPL12 | 1.48E−12 | −0.307672761 | 0.938 | 0.974 | 3.40E−08 | −9.05E+06 |
| CD.Paneth.DEFA6.ITLN2 | ADAMDEC1 | 1.48E−12 | 0.345999979 | 0.293 | 0.043 | 3.41E−08 | 1.02E+07 |
| CD.Bclls.CD69.GPR183 | PFN1 | 1.50E−12 | −0.448270368 | 0.662 | 0.766 | 3.45E−08 | −1.30E+07 |
| CD.Prolif/EC.UBE2C.CCNB2 | HLA-DPA1 | 1.50E−12 | −0.875379165 | 0.51 | 0.896 | 3.45E−08 | −2.54E+07 |
| CD.Fibro.TFPI2.CCL13 | RPL39 | 1.51E−12 | −0.409669808 | 0.977 | 1 | 3.48E−08 | −1.18E+07 |
| CD.Mac.APOE.PTGDS | IGHA1 | 1.51E−12 | 0.295732867 | 0.901 | 0.872 | 3.48E−08 | 8.49E+06 |
| CD.Plsma/IgA.IGLV2-14.IGLV3-19 | RPS13 | 1.52E−12 | 0.642597218 | 0.97 | 0.868 | 3.50E−08 | 1.83E+07 |
| CD.Plsma/IgA.IGLV2-14.IGLV3-19 | RPS4Y1 | 1.54E−12 | 0.55065954 | 0.629 | 0.088 | 3.54E−08 | 1.56E+07 |
| CD.Plsma/IgA.IGKV1-12.IGHA2 | RPS16 | 1.54E−12 | 0.632046808 | 0.972 | 0.966 | 3.55E−08 | 1.78E+07 |
| CD.Bclls.CD69.GPR183 | BAG3 | 1.59E−12 | 0.39181034 | 0.102 | 0.015 | 3.67E−08 | 1.07E+07 |
| CD.Paneth.DEFA6.ITLN2 | HES1 | 1.70E−12 | 0.597101774 | 0.58 | 0.252 | 3.91E−08 | 1.53E+07 |
| CD.Plsma/IgA.AL9287683.RPL39 | FAM89B | 1.70E−12 | −0.267859933 | 0.12 | 0.279 | 3.92E−08 | −6.83E+06 |
| CD.cDC1.CLEC9A.XCR1 | H3F3B | 1.75E−12 | 0.34062092 | 0.994 | 1 | 4.03E−08 | 8.46E+06 |
| CD.cDC1.CLEC9A.XCR1 | SNX3 | 1.83E−12 | −0.300993632 | 0.983 | 1 | 4.22E−08 | −7.14E+06 |
| CD.Fibro.TFPI2.CCL13 | RCN3 | 1.89E−12 | −0.542708043 | 0.018 | 0.204 | 4.34E−08 | −1.25E+07 |
| CD.Fibro.TFPI2.CCL13 | APELA | 1.90E−12 | −0.485132318 | 0.039 | 0.269 | 4.37E−08 | −1.11E+07 |
| CD.Mac.APOE.PTGDS | MT-CO2 | 1.94E−12 | −0.254916393 | 0.854 | 0.892 | 4.48E−08 | −5.70E+06 |
| CD.NK.CCL3.CD160 | UQCRB | 1.99E−12 | 0.373383606 | 0.504 | 0.328 | 4.58E−08 | 8.15E+06 |
| CD.Fibro.TFPI2.CCL13 | EID1 | 2.04E−12 | 0.60734411 | 0.894 | 0.731 | 4.69E−08 | 1.29E+07 |
| CD.cDC1.CLEC9A.XCR1 | XIST | 2.04E−12 | −0.64337084 | 0.145 | 0.485 | 4.70E−08 | −1.37E+07 |
| CD.Plsma/IgA.MT-ND5.MT-ND4L | MTRNR2L12 | 2.12E−12 | 0.987088882 | 0.344 | 0.118 | 4.88E−08 | 2.02E+07 |
| CD.T.CCL20.RORA | HLA-C | 2.16E−12 | 0.28806323 | 0.951 | 0.926 | 4.98E−08 | 5.79E+06 |
| CD.Endth.MT-CO3.MT-CO2 | IGKC | 2.17E−12 | 0.878645949 | 0.976 | 0.897 | 4.99E−08 | 1.76E+07 |
| CD.B.RPS10.FCER2 | RPL8 | 2.18E−12 | −0.347429153 | 0.98 | 0.976 | 5.03E−08 | −6.91E+06 |
| CD.Fibro.TFPI2.CCL13 | HSP90AA1 | 2.23E−12 | 0.649069549 | 0.907 | 0.785 | 5.12E−08 | 1.27E+07 |
| CD.Bclls.CD69.GPR183 | KLF6 | 2.23E−12 | 0.485337314 | 0.756 | 0.508 | 5.14E−08 | 9.45E+06 |
| CD.NK.CCL3.CD160 | CHCHD2 | 2.27E−12 | 0.357430748 | 0.604 | 0.43 | 5.22E−08 | 6.85E+06 |
| CD.B.RPS29.RPS21 | IGHG4 | 2.29E−12 | −0.39066298 | 0.077 | 0.198 | 5.27E−08 | −7.42E+06 |
| CD.Plsma/IgM.IGHM.JCHAIN | PRDX4 | 2.30E−12 | 0.302987873 | 0.84 | 0.78 | 5.29E−08 | 5.72E+06 |
| CD.B.MT-CO2.MT-CO1 | IGHG1 | 2.32E−12 | 0.689507162 | 0.364 | 0.164 | 5.35E−08 | 1.29E+07 |
| CD.Plsma/IgM.IGHM.JCHAIN | IGLC3 | 2.36E−12 | 0.307989491 | 0.704 | 0.567 | 5.44E−08 | 5.67E+06 |
| CD.Mac.APOE.PTGDS | PLIN2 | 2.37E−12 | −0.338295902 | 0.306 | 0.474 | 5.47E−08 | −6.19E+06 |
| CD.Plsma/IgA.IGLV1-51.IGLV10-54 | EEF1A1 | 2.48E−12 | 0.633219965 | 0.981 | 0.972 | 5.71E−08 | 1.11E+07 |
| CD.Mito.MT-CO2.MT-ND2 | RPS4Y1 | 2.49E−12 | 0.564607809 | 0.198 | 0.058 | 5.73E−08 | 9.85E+06 |
| CD.Plsma/IgA.IGLC3.IGLC2.2 | RPS4X | 2.52E−12 | 0.280586717 | 0.995 | 1 | 5.80E−08 | 4.84E+06 |
| CD.Fibro.TFPI2.CCL13 | RPS7 | 2.56E−12 | 0.451953746 | 0.974 | 0.892 | 5.89E−08 | 7.68E+06 |
| CD.Mito.MT-CO2.MT-ND2 | CCL3L3 | 2.62E−12 | 0.690900876 | 0.365 | 0.183 | 6.03E−08 | 1.15E+07 |
| CD.Plsma/IgM.IGHM.JCHAIN | FAM46C | 2.65E−12 | 0.315784619 | 0.728 | 0.582 | 6.09E−08 | 5.18E+06 |
| CD.Paneth.DEFA6.ITLN2 | PRKAR1A | 2.68E−12 | 0.392158638 | 0.576 | 0.22 | 6.17E−08 | 6.35E+06 |
| CD.Fibro.TFPI2.CCL13 | RPL22 | 2.73E−12 | 0.473386602 | 0.938 | 0.892 | 6.28E−08 | 7.54E+06 |
| CD.Plsma/IgA.AL9287683.RPL39 | BASP1 | 2.76E−12 | −0.378340757 | 0.246 | 0.412 | 6.35E−08 | −5.96E+06 |
| CD.T.TRDC.TRGC2 | EEF1A1 | 2.77E−12 | 0.262740021 | 0.997 | 0.972 | 6.38E−08 | 4.12E+06 |
| CD.Plsma/IgA.IGHA2.IGKV1-12 | SEL1L | 2.79E−12 | −0.288587014 | 0.352 | 0.549 | 6.43E−08 | −4.49E+06 |
| CD.Plsma/IgA.IGLL5.IGLV2-14 | IGLL5 | 2.83E−12 | 0.789219586 | 1 | 0.996 | 6.51E−08 | 1.21E+07 |
| CD.Fibro.TFPI2.CCL13 | GNRH2 | 2.85E−12 | −0.366725673 | 0.013 | 0.183 | 6.55E−08 | −5.60E+06 |
| CD.NK.CCL3.CD160 | SRGN | 2.85E−12 | −0.289025506 | 0.935 | 0.938 | 6.57E−08 | −4.40E+06 |
| CD.Plsma/IgA.IGKV1-12.IGHA2 | RPL18 | 2.88E−12 | 0.570941467 | 0.964 | 0.915 | 6.62E−08 | 8.62E+06 |
| CD.B.IGLC3.JCHAIN | EEF1A1 | 2.95E−12 | 0.414072165 | 0.993 | 1 | 6.79E−08 | 6.10E+06 |
| CD.NK.CCL3.CD160 | HIST1H4C | 2.97E−12 | 0.504306891 | 0.215 | 0.093 | 6.85E−08 | 7.36E+06 |
| CD.Fibro.TFPI2.CCL13 | IGFBP4 | 3.04E−12 | −0.793874549 | 0.525 | 0.763 | 6.99E−08 | −1.14E+07 |
| CD.Plsma/IgM.IGHM.JCHAIN | ACTG1 | 3.05E−12 | 0.350243747 | 0.758 | 0.6 | 7.03E−08 | 4.98E+06 |
| CD.Bclls.CD69.GPR183 | TUBB4B | 3.15E−12 | 0.524139704 | 0.263 | 0.098 | 7.26E−08 | 7.22E+06 |
| CD.Plsma/IgA.IGHA2.IGKV1-12 | HLA-DPA1 | 3.18E−12 | 0.280854798 | 0.439 | 0.214 | 7.32E−08 | 3.84E+06 |
| CD.Bclls.CD69.GPR183 | ZFAND2A | 3.22E−12 | 0.640007507 | 0.199 | 0.062 | 7.40E−08 | 8.65E+06 |
| CD.B.ACTB.JCHAIN | EEF1A1 | 3.22E−12 | 0.575629891 | 0.975 | 0.993 | 7.41E−08 | 7.76E+06 |
| CD.Mito.MT-CO2.MT-ND2 | IGKC | 3.40E−12 | 0.405815586 | 0.984 | 0.921 | 7.84E−08 | 5.18E+06 |
| CD.Fibro.TFPI2.CCL13 | CHST2 | 3.69E−12 | −0.298455702 | 0.008 | 0.161 | 8.49E−08 | −3.51E+06 |
| CD.Plsma/IgA.AL9287683.RPL39 | TMEM59 | 3.72E−12 | −0.264291961 | 0.911 | 0.899 | 8.56E−08 | −3.09E+06 |
| CD.B.MT-CO2.MT-CO1 | HLA-B | 3.77E−12 | 0.311168565 | 0.923 | 0.853 | 8.67E−08 | 3.59E+06 |
| CD.B.MT-CO2.MT-CO1 | CD52 | 3.90E−12 | −0.436702549 | 0.799 | 0.881 | 8.98E−08 | −4.86E+06 |
| CD.B.IGHG1.IGLC2 | HLA-DRB1 | 4.01E−12 | 0.45593716 | 0.95 | 0.816 | 9.23E−08 | 4.94E+06 |
| CD.Plsma/IgA.IGHA2.IGKV1-12 | UQCRH | 4.01E−12 | 0.321212659 | 0.708 | 0.507 | 9.24E−08 | 3.48E+06 |
| CD.Fibro.TFPI2.CCL13 | HNRNPA1 | 4.09E−12 | 0.609371739 | 0.91 | 0.806 | 9.41E−08 | 6.48E+06 |
| CD.Plsma/IgA.IGHA1.IGLV6-57 | RPS3A | 4.12E−12 | 0.66617785 | 0.966 | 0.972 | 9.47E−08 | 7.03E+06 |
| CD.Plsma/IgA.AL9287683.RPL39 | S100A6 | 4.15E−12 | −0.346177768 | 0.368 | 0.533 | 9.54E−08 | −3.63E+06 |
| CD.Plsma/IgA.IGHA2.IGKV1-12 | DUSP1 | 4.36E−12 | 0.596973219 | 0.831 | 0.855 | 1.00E−07 | 5.95E+06 |
| CD.T.CCR7.GPR183 | IGKC | 4.39E−12 | 0.362587137 | 0.933 | 0.884 | 1.01E−07 | 3.59E+06 |
| CD.Plsma/IgA.IGHA2.IGKV1-12 | NDUFA1 | 4.41E−12 | −0.278361206 | 0.485 | 0.653 | 1.02E−07 | −2.74E+06 |
| CD.Plsma/IgA.AL9287683.RPL39 | ATP5E | 4.52E−12 | −0.316805228 | 0.911 | 0.917 | 1.04E−07 | −3.04E+06 |
| CD.cDC1.CLEC9A.XCR1 | HSP90AB1 | 4.71E−12 | 0.594046588 | 0.988 | 0.955 | 1.09E−07 | 5.47E+06 |
| CD.Plsma/IgA.IGLL5.IGLV2-14 | RPL11 | 4.74E−12 | 0.302750851 | 1 | 0.977 | 1.09E−07 | 2.78E+06 |
| CD.cDC1.CLEC9A.XCR1 | DNAJA1 | 4.77E−12 | 0.544874785 | 0.96 | 0.939 | 1.10E−07 | 4.97E+06 |
| CD.T.CCR7.SELL | ZFP36 | 4.78E−12 | 0.373681571 | 0.192 | 0.098 | 1.10E−07 | 3.39E+06 |
| CD.T.TRDC.TRGC2 | RPS10 | 4.79E−12 | 0.488823582 | 0.728 | 0.476 | 1.10E−07 | 4.44E+06 |
| CD.Fibro.TFPI2.CCL13 | RPL26 | 4.85E−12 | 0.352519535 | 0.992 | 0.946 | 1.12E−07 | 3.16E+06 |
| CD.B.ACTB.JCHAIN | JCHAIN | 4.94E−12 | 1.035199617 | 0.778 | 0.537 | 1.14E−07 | 9.10E+06 |
| CD.T.MT-CO2.MT-CO1 | MALAT1 | 5.04E−12 | 0.258746629 | 0.997 | 1 | 1.16E−07 | 2.23E+06 |
| CD.NK.CCL3.CD160 | EVL | 5.14E−12 | −0.355105335 | 0.53 | 0.638 | 1.18E−07 | −3.00E+06 |
| CD.Plsma/IgA.IGLC3.IGLC2.2 | SYNGR2 | 5.29E−12 | 0.32025436 | 0.575 | 0.225 | 1.22E−07 | 2.63E+06 |
| CD.NK.CCL3.CD160 | RPS4Y1 | 5.32E−12 | 0.390402479 | 0.295 | 0.151 | 1.22E−07 | 3.19E+06 |
| CD.Plsma/IgM.IGHM.IGLV2-14 | BTG2 | 5.44E−12 | −0.413153779 | 0.687 | 0.85 | 1.25E−07 | −3.30E+06 |
| CD.Plsma/IgA.IGKV1-12.IGHA2 | MT-ND4 | 5.47E−12 | 0.594436386 | 0.986 | 1 | 1.26E−07 | 4.72E+06 |
| CD.B.IGHG1.IGLC2 | IGHG1 | 5.70E−12 | 0.819515373 | 0.61 | 0.272 | 1.31E−07 | 6.24E+06 |
| CD.T.CD8B.TRGC2 | CAPG | 5.88E−12 | 0.57094947 | 0.282 | 0.115 | 1.35E−07 | 4.22E+06 |
| CD.Fibro.TFPI2.CCL13 | SERF2 | 6.00E−12 | −0.457401107 | 0.928 | 0.978 | 1.38E−07 | −3.31E+06 |
| CD.Plsma/IgA.IGHA2.IGKV1-12 | RPL22L1 | 6.33E−12 | 0.263069687 | 0.538 | 0.286 | 1.46E−07 | 1.81E+06 |
| CD.Plsma/IgA.AL9287683.RPL39 | VIM | 6.43E−12 | 0.303435977 | 0.88 | 0.783 | 1.48E−07 | 2.05E+06 |
| CD.B.MT-CO2.MT-CO1 | EEF1D | 6.56E−12 | −0.416327664 | 0.727 | 0.826 | 1.51E−07 | −2.76E+06 |
| CD.Fibro.TFPI2.CCL13 | POLR2L | 6.90E−12 | −0.688732806 | 0.512 | 0.806 | 1.59E−07 | −4.34E+06 |
| CD.cDC1.CLEC9A.XCR1 | ARF6 | 7.23E−12 | −0.474078077 | 0.798 | 0.904 | 1.66E−07 | −2.85E+06 |
| CD.Plsma/IgA.IGKV1-12.IGHA2 | RPL32 | 7.23E−12 | 0.489846814 | 0.989 | 0.983 | 1.66E−07 | 2.94E+06 |
| CD.Plsma/IgA.AL9287683.RPL39 | WARS | 7.24E−12 | −0.47600818 | 0.242 | 0.402 | 1.67E−07 | −2.86E+06 |
| CD.TA.PLCG2.SOX4 | RPS19 | 7.28E−12 | −0.517522634 | 0.859 | 0.97 | 1.67E−07 | −3.09E+06 |
| CD.Plsma/IgM.IGHM.IGLV2-14 | GLTSCR2 | 7.36E−12 | 0.440996352 | 0.668 | 0.455 | 1.69E−07 | 2.60E+06 |
| CD.NK.CCL3.CD160 | TUBA1B | 7.45E−12 | 0.335657039 | 0.444 | 0.272 | 1.71E−07 | 1.96E+06 |
| CD.Plsma/IgA.IGHA1.IGKC | NCL | 7.50E−12 | −0.276956437 | 0.389 | 0.56 | 1.73E−07 | −1.61E+06 |
| CD.T.CD8B.TRGC2 | S100A10 | 7.66E−12 | 0.476457147 | 0.513 | 0.369 | 1.76E−07 | 2.70E+06 |
| CD.Mac.APOE.PTGDS | REG1A | 7.73E−12 | −0.398327006 | 0.111 | 0.248 | 1.78E−07 | −2.24E+06 |
| CD.Plsma/IgA.MT-ND5.MT-ND4L | EEF1A1 | 7.93E−12 | 0.521497581 | 0.891 | 0.767 | 1.83E−07 | 2.86E+06 |
| CD.T.GNLY.IGLC2 | IGHG1 | 8.15E−12 | 1.656899011 | 0.571 | 0.08 | 1.88E−07 | 8.83E+06 |
| CD.Plsma/IgA.IGHA2.IGKV1-12 | UQCR11 | 8.18E−12 | −0.271519737 | 0.415 | 0.606 | 1.88E−07 | −1.44E+06 |
| CD.Mstcl.CTSG.CD69 | MT-ND3 | 8.33E−12 | 0.529352808 | 0.775 | 0.485 | 1.92E−07 | 2.76E+06 |
| CD.Plsma/IgA.IGHA1.IGKC | MT-ND5 | 8.37E−12 | −0.259381263 | 0.762 | 0.885 | 1.93E−07 | −1.35E+06 |
| CD.Plsma/IgA.IGLC3.IGLC2.2 | RPSA | 8.45E−12 | −0.384541441 | 0.919 | 0.945 | 1.95E−07 | −1.98E+06 |
| CD.Plsma/IgM.IGHM.IGLV2-14 | HSPE1 | 8.51E−12 | 0.5612746 | 0.685 | 0.47 | 1.96E−07 | 2.87E+06 |
| CD.T.CD8B.TRGC2 | EEF1D | 8.59E−12 | −0.303036972 | 0.756 | 0.894 | 1.98E−07 | −1.53E+06 |
| CD.Prolif/EC.UBE2C.CCNB2 | DEFA5 | 8.62E−12 | −0.636141848 | 0.255 | 0.816 | 1.98E−07 | −3.21E+06 |
| CD.NK.CCL3.CD160 | KLRB1 | 8.68E−12 | −0.258385666 | 0.866 | 0.901 | 2.00E−07 | −1.29E+06 |
| CD.Plsma/IgA.IGHA2.IGKV1-12 | ZNF706 | 8.83E−12 | 0.271697385 | 0.672 | 0.456 | 2.03E−07 | 1.34E+06 |
| CD.Plsma/IgA.IGKV1-12.IGHA2 | RPL24 | 8.88E−12 | 0.524516122 | 0.952 | 0.949 | 2.04E−07 | 2.57E+06 |
| CD.B.ACTB.JCHAIN | TPT1 | 8.89E−12 | 1.007128275 | 0.818 | 0.701 | 2.05E−07 | 4.92E+06 |
| CD.Fibro.TFPI2.CCL13 | ANGPTL2 | 9.16E−12 | −0.668157923 | 0.178 | 0.516 | 2.11E−07 | −3.17E+06 |
| CD.NK.GNLY.KLRC1 | IFITM1 | 9.37E−12 | −1.274616231 | 0.007 | 0.296 | 2.16E−07 | −5.91E+06 |
| CD.Plsma/IgA.IGLL5.IGLV2-14 | MT-CO1 | 9.88E−12 | 0.282837266 | 0.983 | 0.989 | 2.27E−07 | 1.24E+06 |
| CD.Prolif/EC.UBE2C.CCNB2 | HLA-DRB5 | 1.01E−11 | −0.83803692 | 0.451 | 0.828 | 2.34E−07 | −3.59E+06 |
| CD.Fibro.TFPI2.CCL13 | BCHE | 1.03E−11 | −0.371635956 | 0.028 | 0.226 | 2.36E−07 | −1.57E+06 |
| CD.Plsma/IgA.AL9287683.RPL39 | CTSZ | 1.03E−11 | −0.335020217 | 0.194 | 0.351 | 2.36E−07 | −1.42E+06 |
| CD.Plsma/IgA.IGHA2.IGKV1-12 | CD63 | 1.04E−11 | 0.274740327 | 0.858 | 0.771 | 2.38E−07 | 1.15E+06 |
| CD.Plsma/IgM.IGHM.JCHAIN | SEC11C | 1.07E−11 | 0.256837966 | 0.902 | 0.873 | 2.45E−07 | 1.05E+06 |
| CD.Plsma/IgA.IGLV2-14.IGLV3-19 | RPL26 | 1.08E−11 | 0.628950005 | 0.97 | 0.897 | 2.49E−07 | 2.52E+06 |
| CD.Plsma/IgA.IGLC3.IGLC2.2 | RPS16 | 1.09E−11 | −0.356207681 | 0.995 | 0.995 | 2.50E−07 | −1.42E+06 |
| CD.B.MT-CO2.MT-CO1 | RPS28 | 1.14E−11 | 0.359419693 | 0.933 | 0.905 | 2.63E−07 | 1.36E+06 |
| CD.T.CCL20.RORA | CCL4 | 1.16E−11 | 0.544871533 | 0.323 | 0.14 | 2.67E−07 | 2.04E+06 |
| CD.Mac.APOE.PTGDS | SGK1 | 1.17E−11 | −0.312039954 | 0.408 | 0.57 | 2.69E−07 | −1.16E+06 |
| CD.T.CCL20.RORA | BIRC3 | 1.24E−11 | 0.460899116 | 0.684 | 0.461 | 2.85E−07 | 1.61E+06 |
| CD.Bclls.CD69.GPR183 | CYSTM1 | 1.26E−11 | 0.325871842 | 0.105 | 0.018 | 2.90E−07 | 1.12E+06 |
| CD.Fibro.TFPI2.CCL13 | IFITM3 | 1.28E−11 | −0.446701274 | 0.959 | 0.946 | 2.94E−07 | −1.52E+06 |
| CD.B.IGLC3.JCHAIN | IGLC2 | 1.30E−11 | −1.49136424 | 0.425 | 0.75 | 2.99E−07 | −4.99E+06 |
| CD.Plsma/IgM.IGHM.IGLV2-14 | RPL39 | 1.30E−11 | 0.308346677 | 0.922 | 0.932 | 2.99E−07 | 1.03E+06 |
| CD.Mito.MT-CO2.MT-ND2 | APOC1 | 1.33E−11 | −0.726546992 | 0.157 | 0.329 | 3.07E−07 | −2.37E+06 |
| CD.Plsma/IgG.IGHGP.IGHG2 | LGALS1 | 1.34E−11 | 0.282405033 | 0.644 | 0.577 | 3.09E−07 | 9.14E+05 |
| CD.Plsma/IgA.AL9287683.RPL39 | KLF13 | 1.35E−11 | −0.321909254 | 0.298 | 0.448 | 3.10E−07 | −1.04E+06 |
| CD.B/DZ.RGS13.AICDA | RPS29 | 1.37E−11 | 0.60130678 | 0.992 | 0.908 | 3.14E−07 | 1.91E+06 |
| CD.Plsma/IgA.HSPA1B.HSPA1A | NACA | 1.46E−11 | 0.395154085 | 0.89 | 0.881 | 3.35E−07 | 1.18E+06 |
| CD.B.IGHG1.IGLC2 | RPS14 | 1.47E−11 | 0.305666388 | 0.995 | 0.96 | 3.38E−07 | 9.06E+05 |
| CD.Plsma/IgA.IGHA2.IGKV1-12 | SRPRA | 1.48E−11 | −0.25242455 | 0.189 | 0.377 | 3.40E−07 | −7.42E+05 |
| CD.Plsma/IgM.IGHM.IGLV2-14 | BTF3 | 1.48E−11 | 0.492361824 | 0.701 | 0.56 | 3.40E−07 | 1.45E+06 |
| CD.NK.CCL3.CD160 | RPS17 | 1.51E−11 | 0.257234466 | 0.862 | 0.782 | 3.47E−07 | 7.42E+05 |
| CD.cDC1.CLEC9A.XCR1 | NACA | 1.54E−11 | 0.360653078 | 0.977 | 0.985 | 3.55E−07 | 1.02E+06 |
| CD.T.CD8B.TRGC2 | S100A4 | 1.54E−11 | 0.386386491 | 0.742 | 0.654 | 3.55E−07 | 1.09E+06 |
| CD.Mac.APOE.PTGDS | IGHV3-23 | 1.55E−11 | −0.255707453 | 0.016 | 0.101 | 3.57E−07 | −7.17E+05 |
| CD.Plsma/IgA.IGLC3.IGLC2.1 | RPS4Y1 | 1.56E−11 | 0.296003792 | 0.348 | 0.137 | 3.58E−07 | 8.26E+05 |
| CD.Fibro.TFPI2.CCL13 | NAMPT | 1.58E−11 | −0.632740617 | 0.075 | 0.344 | 3.63E−07 | −1.74E+06 |
| CD.B.MT-CO2.MT-CO1 | RPL38 | 1.58E−11 | 0.42612819 | 0.775 | 0.633 | 3.65E−07 | 1.17E+06 |
| CD.Prolif/EC.UBE2C.CCNB2 | IGLC2 | 1.61E−11 | 1.003186828 | 0.882 | 0.772 | 3.70E−07 | 2.71E+06 |
| CD.Bclls.CD69.GPR183 | RHOB | 1.61E−11 | 0.46282891 | 0.199 | 0.063 | 3.71E−07 | 1.25E+06 |
| CD.Plsma/IgA.IGLL5.IGLV2-14 | RPL7 | 1.63E−11 | 0.251572083 | 0.987 | 0.996 | 3.75E−07 | 6.71E+05 |
| CD.Mac.APOE.PTGDS | TFRC | 1.68E−11 | −0.305741329 | 0.306 | 0.461 | 3.88E−07 | −7.88E+05 |
| CD.Plsma/IgA.IGHA1.IGKC | IER2 | 1.73E−11 | −0.37397943 | 0.656 | 0.785 | 3.98E−07 | −9.40E+05 |
| CD.B.MT-ND4L.IGHG1 | MT-ND4L | 1.77E−11 | −1.125607374 | 0.254 | 0.669 | 4.08E−07 | −2.76E+06 |
| CD.cDC1.CLEC9A.XCR1 | EIF5 | 1.78E−11 | 0.49729516 | 0.913 | 0.783 | 4.09E−07 | 1.22E+06 |
| CD.Plsma/IgA.IGHA2.IGKV1-12 | SEC61G | 1.79E−11 | −0.266095435 | 0.778 | 0.874 | 4.12E−07 | −6.46E+05 |
| CD.Fibro.TFPI2.CCL13 | COL3A1 | 1.82E−11 | −0.854943767 | 0.798 | 0.935 | 4.20E−07 | −2.04E+06 |
| CD.NK.CCL3.CD160 | SAP18 | 1.90E−11 | 0.348748978 | 0.449 | 0.284 | 4.36E−07 | 7.99E+05 |
| CD.Mono.S100A8.S100A9 | TPT1 | 1.93E−11 | 0.600326656 | 0.99 | 0.95 | 4.44E−07 | 1.35E+06 |
| CD.T.MT-CO2.MT-CO3 | B2M | 1.96E−11 | −0.314118346 | 0.987 | 0.993 | 4.52E−07 | −6.95E+05 |
| CD.B.RPS29.RPS21 | EIF5A | 2.07E−11 | −0.385470674 | 0.147 | 0.284 | 4.77E−07 | −8.08E+05 |
| CD.Plsma/IgA.IGKV1-12.IGHA2 | RPS14 | 2.10E−11 | 0.488378284 | 0.966 | 1 | 4.84E−07 | 1.01E+06 |
| CD.TA.PLCG2.SOX4 | RPL8 | 2.15E−11 | −0.608012113 | 0.66 | 0.839 | 4.94E−07 | −1.23E+06 |
| CD.Plsma/IgA.IGKV1-12.IGHA2 | H3F3B | 2.15E−11 | 0.788672599 | 0.961 | 0.932 | 4.95E−07 | 1.59E+06 |
| CD.Plsma/IgA.IGLC3.IGLC2.2 | TFF3 | 2.16E−11 | −0.325559907 | 0.011 | 0.24 | 4.97E−07 | −6.55E+05 |
| CD.Mono.FCN1.S100A4 | B2M | 2.17E−11 | −0.411471606 | 1 | 1 | 5.00E−07 | −8.23E+05 |
| CD.T.CCL20.RORA | MT2A | 2.21E−11 | 0.476067808 | 0.452 | 0.245 | 5.09E−07 | 9.34E+05 |
| CD.Fibro.TFPI2.CCL13 | SRP14 | 2.23E−11 | 0.475768009 | 0.938 | 0.839 | 5.14E−07 | 9.25E+05 |
| CD.Plsma/IgA.IGHA1.IGKC | DNAJB1 | 2.24E−11 | −0.2642511 | 0.413 | 0.609 | 5.17E−07 | −5.11E+05 |
| CD.Paneth.DEFA6.ITLN2 | IGKV4-1 | 2.29E−11 | 0.343125722 | 0.259 | 0.039 | 5.28E−07 | 6.50E+05 |
| CD.Plsma/IgM.IGHM.IGLV2-14 | EIF1 | 2.30E−11 | 0.364504608 | 0.954 | 0.925 | 5.29E−07 | 6.89E+05 |
| CD.Plsma/IgA.IGHA2.IGKV1-12 | IGHV3-15 | 2.37E−11 | −1.855996171 | 0.167 | 0.342 | 5.44E−07 | −3.41E+06 |
| CD.Plsma/IgA.AL9287683.RPL39 | MANF | 2.42E−11 | −0.280703846 | 0.861 | 0.856 | 5.58E−07 | −5.03E+05 |
| CD.Fibro.TFPI2.CCL13 | SULF1 | 2.42E−11 | −0.713265981 | 0.165 | 0.505 | 5.58E−07 | −1.28E+06 |
| CD.Plsma/IgA.HSPA1B.HSPA1A | RPS20 | 2.44E−11 | 0.389518512 | 0.944 | 0.96 | 5.61E−07 | 6.94E+05 |
| CD.Bclls.CD69.GPR183 | KDM6B | 2.44E−11 | 0.584280956 | 0.346 | 0.162 | 5.62E−07 | 1.04E+06 |
| CD.Paneth.DEFA6.ITLN2 | MT-ND4L | 2.45E−11 | 0.676308365 | 0.878 | 0.709 | 5.64E−07 | 1.20E+06 |
| CD.Fibro.TFPI2.CCL13 | IGHM | 2.45E−11 | 0.62506575 | 0.871 | 0.753 | 5.65E−07 | 1.11E+06 |
| CD.Plsma/IgA.HSPA1B.HSPA1A | RPS17 | 2.48E−11 | 0.327566154 | 0.949 | 0.98 | 5.71E−07 | 5.74E+05 |
| CD.Fibro.TFPI2.CCL13 | MT-ND3 | 2.56E−11 | −0.464725994 | 0.99 | 0.978 | 5.88E−07 | −7.90E+05 |
| CD.Mac.APOE.PTGDS | RSL1D1 | 2.59E−11 | −0.252816686 | 0.247 | 0.388 | 5.96E−07 | −4.24E+05 |
| CD.Plsma/IgA.IGLC3.IGLC2.1 | PRDX4 | 2.61E−11 | 0.368126652 | 0.886 | 0.852 | 6.00E−07 | 6.14E+05 |
| CD.Mac.APOE.PTGDS | TCOF1 | 2.62E−11 | 0.282441617 | 0.159 | 0.068 | 6.03E−07 | 4.69E+05 |
| CD.Plsma/IgA.IGHA1.IGLV6-57 | MT-CO3 | 2.63E−11 | 0.496229157 | 0.94 | 0.978 | 6.06E−07 | 8.19E+05 |
| CD.Plsma/IgM.IGHM.IGLV2-14 | RPS11 | 2.72E−11 | 0.401169407 | 0.898 | 0.895 | 6.25E−07 | 6.41E+05 |
| CD.Plsma/IgM.IGHM.IGLV2-14 | RPS17 | 2.72E−11 | 0.360108079 | 0.908 | 0.898 | 6.27E−07 | 5.74E+05 |
| CD.Paneth.DEFA6.ITLN2 | CXCL1 | 2.73E−11 | 0.304257068 | 0.185 | 0.008 | 6.27E−07 | 4.85E+05 |
| CD.Plsma/IgA.IGLC3.IGLC2.2 | PABPC1 | 2.81E−11 | 0.40573565 | 0.882 | 0.67 | 6.46E−07 | 6.28E+05 |
| CD.Plsma/IgA.IGLC3.IGLC2.1 | DUSP5 | 2.84E−11 | −0.384193309 | 0.343 | 0.593 | 6.54E−07 | −5.88E+05 |
| CD.Plsma/IgG.IGHGP.IGHG2 | PTP4A3 | 2.85E−11 | 0.269586372 | 0.215 | 0.137 | 6.56E−07 | 4.11E+05 |
| CD.Plsma/IgA.IGLC3.IGLC2.1 | HSPA1B | 2.90E−11 | −0.606350728 | 0.199 | 0.467 | 6.68E−07 | −9.08E+05 |
| CD.B.MT-CO2.MT-CO1 | RPL30 | 2.90E−11 | 0.363436385 | 0.88 | 0.88 | 6.68E−07 | 5.44E+05 |
| CD.T.CCR7.SELL | H1FX | 2.92E−11 | −0.462185912 | 0.04 | 0.114 | 6.71E−07 | −6.88E+05 |
| CD.Fibro.TFPI2.CCL13 | SFRP1 | 2.97E−11 | −0.843666999 | 0.649 | 0.828 | 6.84E−07 | −1.23E+06 |
| CD.cDC1.CLEC9A.XCR1 | RPS24 | 2.99E−11 | 0.276501292 | 0.994 | 1 | 6.89E−07 | 4.01E+05 |
| CD.Plsma/IgA.IGHA1.IGLV6-57 | RPL26 | 3.07E−11 | 0.473792006 | 0.957 | 0.963 | 7.07E−07 | 6.70E+05 |
| CD.Plsma/IgA.IGKV1-12.IGHA2 | RPS13 | 3.10E−11 | 0.527177586 | 0.961 | 0.949 | 7.14E−07 | 7.39E+05 |
| CD.cDC1.CLEC9A.XCR1 | UQCRB | 3.11E−11 | 0.347404442 | 0.977 | 0.899 | 7.17E−07 | 4.85E+05 |
| CD.T.CCL20.RORA | RPL37A | 3.12E−11 | 0.332258462 | 0.856 | 0.754 | 7.17E−07 | 4.63E+05 |
| CD.EC.MTRNR2L1.MT-ND3 | HLA-B | 3.15E−11 | −0.705969249 | 0.556 | 0.673 | 7.25E−07 | −9.73E+05 |
| CD.Plsma/IgA.IGLV2-14.IGLV3-19 | RPS5 | 3.22E−11 | 0.58865997 | 0.994 | 0.926 | 7.42E−07 | 7.93E+05 |
| CD.Plsma/IgM.IGHM.IGLV2-14 | RPL13A | 3.33E−11 | 0.281078614 | 0.976 | 0.959 | 7.66E−07 | 3.67E+05 |
| CD.Plsma/IgA.AL9287683.RPL39 | DUSP1 | 3.33E−11 | 0.443496094 | 0.813 | 0.701 | 7.67E−07 | 5.78E+05 |
| CD.Mac.APOE.PTGDS | PLEKHO1 | 3.41E−11 | −0.355413976 | 0.263 | 0.403 | 7.86E−07 | −4.52E+05 |
| CD.Plsma/IgM.IGHM.JCHAIN | CD69 | 3.43E−11 | 0.285027575 | 0.416 | 0.222 | 7.90E−07 | 3.61E+05 |
| CD.B.MT-CO2.MT-CO1 | HSPA8 | 3.43E−11 | 0.611306269 | 0.55 | 0.346 | 7.91E−07 | 7.73E+05 |
| CD.Mac.APOE.PTGDS | AREG | 3.49E−11 | −0.414915229 | 0.13 | 0.263 | 8.03E−07 | −5.17E+05 |
| CD.Plsma/IgA.IGHA2.IGKV1-12 | FOS | 3.49E−11 | −0.425459228 | 0.854 | 0.916 | 8.03E−07 | −5.30E+05 |
| CD.Plsma/IgA.IGLC3.IGLC2.1 | EEF1A1 | 3.58E−11 | 0.561470881 | 0.985 | 0.956 | 8.23E−07 | 6.82E+05 |
| CD.Plsma/IgA.IGLC3.IGLC2.2 | RPS19 | 3.69E−11 | −0.316018717 | 1 | 1 | 8.50E−07 | −3.72E+05 |
| CD.Plsma/IgA.IGLC3.IGLC2.2 | VIM | 3.76E−11 | 0.447078179 | 0.946 | 0.815 | 8.65E−07 | 5.17E+05 |
| CD.Plsma/IgA.IGLV2-14.IGLV3-19 | RPS17 | 3.77E−11 | 0.592289153 | 0.976 | 0.882 | 8.67E−07 | 6.83E+05 |
| CD.Plsma/IgA.IGHA1.IGKC | MTHFD2 | 3.77E−11 | −0.280905696 | 0.225 | 0.387 | 8.68E−07 | −3.24E+05 |
| CD.Plsma/IgA.IGLV2-14.IGLV3-19 | RPL21 | 3.83E−11 | 0.511979 | 0.982 | 0.985 | 8.82E−07 | 5.81E+05 |
| CD.B.IGLC3.JCHAIN | MT-ND3 | 3.87E−11 | 0.555962772 | 0.979 | 0.905 | 8.91E−07 | 6.24E+05 |
| CD.Mono.S100A8.S100A9 | RPS13 | 3.95E−11 | 0.461357216 | 0.952 | 0.886 | 9.10E−07 | 5.07E+05 |
| CD.cDC1.CLEC9A.XCR1 | ACTG1 | 4.00E−11 | 0.35269734 | 1 | 1 | 9.20E−07 | 3.83E+05 |
| CD.Plsma/IgA.IGLC3.IGLC2.2 | MZT2B | 4.08E−11 | −0.42650788 | 0.554 | 0.725 | 9.40E−07 | −4.54E+05 |
| CD.Fibro.TFPI2.CCL13 | VEGFA | 4.11E−11 | −0.361609777 | 0.023 | 0.204 | 9.46E−07 | −3.82E+05 |
| CD.Fibro.TFPI2.CCL13 | MMD | 4.14E−11 | −0.450331112 | 0.109 | 0.409 | 9.54E−07 | −4.72E+05 |
| CD.T.CARD16.GB2 | MT-ND1 | 4.18E−11 | 0.428917296 | 0.668 | 0.458 | 9.62E−07 | 4.46E+05 |
| CD.T.CCL4.GMZK | PTPRC | 4.19E−11 | −0.344374098 | 0.716 | 0.784 | 9.64E−07 | −3.57E+05 |
| CD.Plsma/IgA.IGLC3.IGLC2.2 | RPL13A | 4.23E−11 | −0.252834083 | 1 | 1 | 9.73E−07 | −2.60E+05 |
| CD.B.MT-CO2.MT-CO1 | KLF6 | 4.23E−11 | 0.491057116 | 0.598 | 0.351 | 9.74E−07 | 5.04E+05 |
| CD.T.CD8B.TRGC2 | ACTG1 | 4.36E−11 | 0.340608848 | 0.746 | 0.631 | 1.00E−06 | 3.39E+05 |
| CD.NK.CCL3.CD160 | TYROBP | 4.40E−11 | −0.274203931 | 0.767 | 0.828 | 1.01E−06 | −2.71E+05 |
| CD.NK.GNLY.IFNG | IGKC | 4.42E−11 | 0.711477009 | 0.974 | 0.955 | 1.02E−06 | 6.99E+05 |
| CD.Bclls.CD69.GPR183 | CD83 | 4.44E−11 | 0.454084764 | 0.812 | 0.694 | 1.02E−06 | 4.45E+05 |
| CD.TA.PLCG2.SOX4 | RPL13 | 4.64E−11 | −0.491008895 | 0.891 | 0.97 | 1.07E−06 | −4.59E+05 |
| CD.Fibro.TFPI2.CCL13 | POSTN | 4.66E−11 | −0.965960447 | 0.054 | 0.28 | 1.07E−06 | −9.01E+05 |
| CD.Fibro.TFPI2.CCL13 | IL6ST | 4.66E−11 | −0.656078264 | 0.525 | 0.817 | 1.07E−06 | −6.11E+05 |
| CD.Goblet.REG4.SPINK4 | PPDPF | 4.67E−11 | −1.539866571 | 0.312 | 0.797 | 1.08E−06 | −1.43E+06 |
| CD.Bclls.CD69.GPR183 | YME1L1 | 4.76E−11 | 0.45574308 | 0.211 | 0.071 | 1.10E−06 | 4.16E+05 |
| CD.Fibro.TFPI2.CCL13 | ITGB1 | 4.77E−11 | −0.672930844 | 0.429 | 0.763 | 1.10E−06 | −6.13E+05 |
| CD.NK.CCL3.CD160 | MRFAP1 | 4.87E−11 | 0.444736714 | 0.238 | 0.117 | 1.12E−06 | 3.97E+05 |
| CD.Fibro.RERGL.NTRK2 | IGKC | 4.96E−11 | 0.786483222 | 1 | 0.92 | 1.14E−06 | 6.89E+05 |
| CD.Plsma/IgA.IGLV1-51.IGLV10-54 | TPT1 | 4.99E−11 | 0.600627975 | 1 | 0.961 | 1.15E−06 | 5.23E+05 |
| CD.Plsma/IgA.MT-ND5.MT-ND4L | MT-ND1 | 5.02E−11 | 0.764064585 | 0.94 | 0.919 | 1.16E−06 | 6.61E+05 |
| CD.T.CD8B.TRGC2 | PSMD5-AS1 | 5.14E−11 | −0.379565719 | 0.028 | 0.115 | 1.18E−06 | −3.21E+05 |
| CD.T.CCL20.RORA | CSF2 | 5.15E−11 | 0.53512881 | 0.103 | 0.019 | 1.19E−06 | 4.51E+05 |
| CD.Plsma/IgA.IGKV1-12.IGHA2 | RPL19 | 5.22E−11 | 0.530627606 | 0.961 | 0.932 | 1.20E−06 | 4.42E+05 |
| CD.Plsma/IgA.IGHA2.IGKV1-12 | IGHV3-23 | 5.26E−11 | −0.426143594 | 0.263 | 0.448 | 1.21E−06 | −3.52E+05 |
| CD.cDC1.CLEC9A.XCR1 | MTRNR2L12 | 5.27E−11 | 0.462439898 | 0.329 | 0.066 | 1.21E−06 | 3.81E+05 |
| CD.Bclls.CD69.GPR183 | MT-CO3 | 5.31E−11 | 0.254974873 | 0.97 | 0.976 | 1.22E−06 | 2.09E+05 |
| CD.Plsma/IgG.IGHGP.IGHG2 | MTRNR2L8 | 5.35E−11 | 0.25705873 | 0.16 | 0.089 | 1.23E−06 | 2.09E+05 |
| CD.T.CD8B.TRGC2 | XCL2 | 5.38E−11 | 0.576376777 | 0.237 | 0.087 | 1.24E−06 | 4.66E+05 |
| CD.Plsma/IgA.IGLC3.IGLC2.2 | KLF2 | 5.38E−11 | −0.559735557 | 0.301 | 0.57 | 1.24E−06 | −4.52E+05 |
| CD.NK.CCL3.CD160 | HLA-DRB1 | 5.71E−11 | −0.501129585 | 0.11 | 0.221 | 1.31E−06 | −3.81E+05 |
| CD.Plsma/IgM.IGHM.JCHAIN | HSPD1 | 5.76E−11 | 0.384307975 | 0.571 | 0.402 | 1.32E−06 | 2.90E+05 |
| CD.Plsma/IgM.IGHM.JCHAIN | KRTCAP2 | 5.76E−11 | 0.267874714 | 0.588 | 0.416 | 1.33E−06 | 2.02E+05 |
| CD.Bclls.CD69.GPR183 | MYADM | 5.78E−11 | 0.556391592 | 0.271 | 0.112 | 1.33E−06 | 4.19E+05 |
| CD.EC.MT1H.MT1G | DEFA6 | 5.81E−11 | −1.032778961 | 0.026 | 0.403 | 1.34E−06 | −7.72E+05 |
| CD.Plsma/IgA.IGHA2.IGKV1-12 | IGKV1-9 | 5.88E−11 | 0.761899669 | 0.203 | 0.052 | 1.35E−06 | 5.63E+05 |
| CD.Plsma/IgM.IGHM.JCHAIN | ATF4 | 5.92E−11 | 0.327229178 | 0.796 | 0.696 | 1.36E−06 | 2.40E+05 |
| CD.T.MAF.CTLA4 | RPL39 | 5.96E−11 | 0.413114011 | 1 | 0.982 | 1.37E−06 | 3.01E+05 |
| CD.T.MT-CO2.MT-CO3 | ACTB | 6.00E−11 | −0.319398296 | 0.869 | 0.946 | 1.38E−06 | −2.31E+05 |
| CD.Mstcl.CTSG.CD69 | TMSB4X | 6.06E−11 | 0.268379933 | 0.993 | 0.973 | 1.39E−06 | 1.92E+05 |
| CD.Bclls.CD69.GPR183 | FAM133B | 6.16E−11 | 0.44001048 | 0.361 | 0.165 | 1.42E−06 | 3.10E+05 |
| CD.Plsma/IgA.AL9287683.RPL39 | DNAJC1 | 6.22E−11 | −0.33923293 | 0.492 | 0.598 | 1.43E−06 | −2.37E+05 |
| CD.Fibro.TFPI2.CCL13 | PPP1CB | 6.37E−11 | −0.500019547 | 0.119 | 0.419 | 1.47E−06 | −3.41E+05 |
| CD.Mono.CXCL10.TNF | PCBP2 | 6.46E−11 | −0.474024964 | 0.189 | 0.588 | 1.49E−06 | −3.19E+05 |
| CD.B.IGLC3.JCHAIN | RPL37A | 6.48E−11 | 0.527409892 | 0.952 | 0.905 | 1.49E−06 | 3.54E+05 |
| CD.Mac.APOE.PTGDS | ZNF331 | 6.50E−11 | −0.279037024 | 0.398 | 0.564 | 1.50E−06 | −1.86E+05 |
| CD.B.IGHG1.IGLC2 | AC090498.1 | 6.71E−11 | −0.894776221 | 0.058 | 0.224 | 1.54E−06 | −5.79E+05 |
| CD.T.CCL20.RORA | EIF1AY | 6.71E−11 | 0.402114929 | 0.114 | 0.025 | 1.55E−06 | 2.60E+05 |
| CD.Plsma/IgA.IGHA1.IGLV6-57 | RPS23 | 6.93E−11 | 0.581335413 | 0.966 | 0.95 | 1.59E−06 | 3.65E+05 |
| CD.Plsma/IgA.IGLV2-14.IGLV3-19 | RPS7 | 6.93E−11 | 0.575244994 | 0.946 | 0.809 | 1.60E−06 | 3.61E+05 |
| CD.Plsma/IgM.IGHM.IGLV2-14 | SEC61B | 6.94E−11 | 0.426672793 | 0.83 | 0.842 | 1.60E−06 | 2.67E+05 |
| CD.Plsma/IgM.IGHM.IGLV2-14 | RPL18 | 7.05E−11 | 0.336608734 | 0.887 | 0.895 | 1.62E−06 | 2.08E+05 |
| CD.Plsma/IgM.IGHM.IGLV2-14 | RP11-16E12.2 | 7.14E−11 | −0.267989707 | 0.067 | 0.248 | 1.64E−06 | −1.63E+05 |
| CD.Plsma/IgA.IGLV2-14.IGLV3-19 | RPL29 | 7.18E−11 | 0.547776159 | 0.976 | 0.882 | 1.65E−06 | 3.31E+05 |
| CD.Plsma/IgM.IGHM.JCHAIN | LMNA | 7.31E−11 | 0.486687433 | 0.508 | 0.353 | 1.68E−06 | 2.89E+05 |
| CD.Prolif/EC.UBE2C.CCNB2 | CD74 | 7.34E−11 | −0.743425859 | 0.922 | 0.992 | 1.69E−06 | −4.40E+05 |
| CD.Plsma/IgA.AL9287683.RPL39 | RABAC1 | 7.41E−11 | −0.287287388 | 0.819 | 0.827 | 1.71E−06 | −1.68E+05 |
| CD.Plsma/IgA.IGHA2.IGKV1-12 | UQCRQ | 7.42E−11 | −0.272843451 | 0.589 | 0.719 | 1.71E−06 | −1.60E+05 |
| CD.T.CCL20.RORA | RPL30 | 7.43E−11 | 0.282352439 | 0.905 | 0.855 | 1.71E−06 | 1.65E+05 |
| CD.EC.OLFM4.MT-ND2 | IGHG1 | 7.51E−11 | 1.223908344 | 0.579 | 0.137 | 1.73E−06 | 7.08E+05 |
| CD.T.CCL4.GMZK | REG1A | 7.59E−11 | −0.571979661 | 0.116 | 0.233 | 1.75E−06 | −3.27E+05 |
| CD.Paneth.DEFA6.ITLN2 | RPL28 | 7.61E−11 | −0.534364664 | 0.844 | 0.902 | 1.75E−06 | −3.05E+05 |
| CD.Plsma/IgA.IGHA2.IGKV1-12 | UFM1 | 7.72E−11 | −0.319188854 | 0.43 | 0.567 | 1.78E−06 | −1.80E+05 |
| CD.Plsma/IgA.IGKV1-12.IGHA2 | RPL6 | 7.75E−11 | 0.497886292 | 0.972 | 0.949 | 1.78E−06 | 2.79E+05 |
| CD.Plsma/IgA.IGLV1-51.IGLV10-54 | RPL30 | 7.78E−11 | 0.545046828 | 0.971 | 0.834 | 1.79E−06 | 3.04E+05 |
| CD.T.CARD16.GB2 | MT-CYB | 7.99E−11 | 0.312915554 | 0.886 | 0.719 | 1.84E−06 | 1.70E+05 |
| CD.T.CCL4.GMZK | ZFP36L2 | 8.06E−11 | 0.334832757 | 0.656 | 0.479 | 1.85E−06 | 1.81E+05 |
| CD.cDC1.CLEC9A.XCR1 | MALAT1 | 8.17E−11 | 0.429079113 | 1 | 1 | 1.88E−06 | 2.28E+05 |
| CD.Plsma/IgM.IGHM.IGLV2-14 | XBP1 | 8.22E−11 | −0.38027618 | 0.863 | 0.955 | 1.89E−06 | −2.01E+05 |
| CD.NK.GNLY.KLRC1 | EIF5A | 8.32E−11 | −1.104929504 | 0.103 | 0.467 | 1.91E−06 | −5.77E+05 |
| CD.Fibro.TFPI2.CCL13 | ARRDC3 | 8.36E−11 | −0.47283308 | 0.145 | 0.473 | 1.92E−06 | −2.46E+05 |
| CD.Fibro.TFPI2.CCL13 | C7orf55 | 8.39E−11 | −0.377390722 | 0.039 | 0.247 | 1.93E−06 | −1.95E+05 |
| CD.Prolif/EC.UBE2C.CCNB2 | HLA-DRA | 8.60E−11 | −0.623422819 | 0.882 | 0.988 | 1.98E−06 | −3.15E+05 |
| CD.Plsma/IgA.IGKV1-12.IGHA2 | RPS7 | 8.74E−11 | 0.489403515 | 0.969 | 0.966 | 2.01E−06 | 2.43E+05 |
| CD.Mono.CXCL10.TNF | SNHG25 | 9.15E−11 | −0.559665207 | 0.03 | 0.307 | 2.11E−06 | −2.66E+05 |
| CD.Bclls.CD69.GPR183 | AHSA1 | 9.21E−11 | 0.43797675 | 0.323 | 0.144 | 2.12E−06 | 2.07E+05 |
| CD.NK.CCL3.CD160 | SRSF7 | 9.31E−11 | 0.320990444 | 0.637 | 0.486 | 2.14E−06 | 1.50E+05 |
| CD.B.MT-CO2.MT-CO1 | MRPL18 | 9.35E−11 | 0.696132942 | 0.244 | 0.094 | 2.15E−06 | 3.23E+05 |
| CD.Bclls.CD69.GPR183 | CD52 | 9.44E−11 | −0.35032385 | 0.741 | 0.851 | 2.17E−06 | −1.61E+05 |
| CD.Plsma/IgA.IGLL5.IGLV2-14 | TIMP1 | 9.53E−11 | 0.353257965 | 0.381 | 0.125 | 2.19E−06 | 1.61E+05 |
| CD.Mono.CXCL10.TNF | HSPA1A | 9.88E−11 | −0.841127308 | 0.348 | 0.699 | 2.27E−06 | −3.70E+05 |
| CD.NK.GNLY.FCER1G | RPS26 | 9.95E−11 | 0.970497773 | 0.863 | 0.488 | 2.29E−06 | 4.24E+05 |
| CD.Mac.APOE.PTGDS | ISG15 | 1.01E−10 | −0.282359502 | 0.312 | 0.466 | 2.32E−06 | −1.22E+05 |
| CD.Bclls.CD69.GPR183 | LTB | 1.01E−10 | −0.458906265 | 0.665 | 0.777 | 2.32E−06 | −1.98E+05 |
| CD.Plsma/IgM.IGHM.JCHAIN | PNRC1 | 1.02E−10 | 0.268966238 | 0.667 | 0.502 | 2.34E−06 | 1.15E+05 |
| CD.Plsma/IgA.IGHA1.IGLV6-57 | RPS20 | 1.02E−10 | 0.623569143 | 0.915 | 0.904 | 2.35E−06 | 2.65E+05 |
| CD.Plsma/IgA.IGKV1-12.IGHA2 | RPS11 | 1.04E−10 | 0.594916239 | 0.936 | 0.915 | 2.40E−06 | 2.48E+05 |
| CD.Bclls.CD69.GPR183 | SLC25A3 | 1.05E−10 | 0.430346874 | 0.406 | 0.208 | 2.42E−06 | 1.78E+05 |
| CD.T.TRDC.TRGC2 | RPL30 | 1.09E−10 | 0.2532938 | 0.951 | 0.864 | 2.50E−06 | 1.01E+05 |
| CD.T.CCR7.GPR183 | PFN1 | 1.09E−10 | 0.259985894 | 0.748 | 0.647 | 2.51E−06 | 1.03E+05 |
| CD.Plsma/IgA.AL9287683.RPL39 | TMBIM6 | 1.10E−10 | −0.279594439 | 0.765 | 0.781 | 2.52E−06 | −1.11E+05 |
| CD.B.IGLC3.JCHAIN | RPS27 | 1.13E−10 | 0.349104923 | 0.993 | 1 | 2.59E−06 | 1.35E+05 |
| CD.NK.CCL3.CD160 | ACTG1 | 1.16E−10 | 0.302869206 | 0.707 | 0.568 | 2.66E−06 | 1.14E+05 |
| CD.Fibro.TFPI2.CCL13 | TRAM1 | 1.18E−10 | −0.490595578 | 0.189 | 0.538 | 2.72E−06 | −1.81E+05 |
| CD.Fibro.TFPI2.CCL13 | RPL37A | 1.21E−10 | −0.489545819 | 0.855 | 0.892 | 2.78E−06 | −1.76E+05 |
| CD.Mac.APOE.PTGDS | YPEL5 | 1.21E−10 | −0.26116878 | 0.311 | 0.46 | 2.80E−06 | −9.34E+04 |
| CD.T.KLRB1.LTF | MT-ND1 | 1.24E−10 | 0.325539796 | 0.834 | 0.731 | 2.85E−06 | 1.14E+05 |
| CD.Mono.FCN1.S100A4 | IGHG1 | 1.25E−10 | 1.262579322 | 0.453 | 0.167 | 2.88E−06 | 4.39E+05 |
| CD.Plsma/IgA.IGHA2.IGKV1-12 | RPS7 | 1.31E−10 | 0.261428258 | 0.917 | 0.929 | 3.01E−06 | 8.68E+04 |
| CD.Plsma/IgA.IGLL5.IGLV2-14 | MTRNR2L8 | 1.32E−10 | 0.371107981 | 0.331 | 0.106 | 3.05E−06 | 1.22E+05 |
| CD.Plsma/IgM.IGHM.IGLV2-14 | RPL11 | 1.33E−10 | 0.301402252 | 0.935 | 0.959 | 3.07E−06 | 9.82E+04 |
| CD.Fibro.RERGL.NTRK2 | ZFP36 | 1.35E−10 | 2.186810559 | 0.571 | 0.114 | 3.10E−06 | 7.06E+05 |
| CD.Plsma/IgA.IGHA2.IGKV1-12 | PIM2 | 1.37E−10 | −0.260381066 | 0.708 | 0.84 | 3.16E−06 | −8.23E+04 |
| CD.Prolif/EC.UBE2C.CCNB2 | HLA-C | 1.41E−10 | −0.527163687 | 0.882 | 0.984 | 3.24E−06 | −1.63E+05 |
| CD.B.MT-CO2.MT-CO1 | HSPA1A | 1.48E−10 | 0.618412523 | 0.196 | 0.066 | 3.41E−06 | 1.81E+05 |
| CD.Plsma/IgA.AL9287683.RPL39 | SRRM2 | 1.49E−10 | −0.319976004 | 0.375 | 0.487 | 3.42E−06 | −9.36E+04 |
| CD.Plsma/IgA.IGKV1-12.IGHA2 | RPL21 | 1.49E−10 | 0.446874749 | 0.986 | 0.983 | 3.43E−06 | 1.30E+05 |
| CD.B/DZ.RGS13.AICDA | RPS27 | 1.51E−10 | 0.392339256 | 1 | 1 | 3.48E−06 | 1.13E+05 |
| CD.T.GNLY.IGLC2 | RPS26 | 1.51E−10 | 0.841811634 | 0.874 | 0.627 | 3.49E−06 | 2.42E+05 |
| CD.Plsma/IgA.IGLC3.IGLC2.2 | TOMM7 | 1.52E−10 | −0.448087181 | 0.774 | 0.84 | 3.50E−06 | −1.28E+05 |
| CD.Plsma/IgA.IGLL5.IGLV2-14 | AL928768.3 | 1.53E−10 | 0.47911266 | 0.9 | 0.693 | 3.53E−06 | 1.36E+05 |
| CD.Plsma/IgA.IGLV2-14.IGLV3-19 | RPL28 | 1.54E−10 | 0.538744268 | 0.982 | 0.956 | 3.55E−06 | 1.52E+05 |
| CD.Plsma/IgA.IGLV1-51.IGLV10-54 | MT-CO3 | 1.55E−10 | 0.501329161 | 0.99 | 0.972 | 3.57E−06 | 1.41E+05 |
| CD.NK.GNLY.FCER1G | IGKC | 1.59E−10 | 0.807743182 | 0.98 | 0.86 | 3.67E−06 | 2.20E+05 |
| CD.Plsma/IgA.IGLV2-14.IGLV3-19 | RPS12 | 1.62E−10 | 0.563672842 | 0.988 | 0.956 | 3.73E−06 | 1.51E+05 |
| CD.Fibro.TFPI2.CCL13 | SPTSSA | 1.63E−10 | −0.601736553 | 0.323 | 0.688 | 3.75E−06 | −1.60E+05 |
| CD.Plsma/IgA.IGLL5.IGLV2-14 | EAF2 | 1.64E−10 | 0.399492079 | 0.77 | 0.564 | 3.77E−06 | 1.06E+05 |
| CD.cDC1.CLEC9A.XCR1 | IGKC | 1.64E−10 | 0.543558688 | 1 | 0.98 | 3.78E−06 | 1.44E+05 |
| CD.Plsma/IgA.IGHA1.IGKC | IGHV3-15 | 1.64E−10 | −1.35902031 | 0.061 | 0.2 | 3.78E−06 | −3.60E+05 |
| CD.Plsma/IgA.AL9287683.RPL39 | SSR3 | 1.70E−10 | −0.269886627 | 0.858 | 0.877 | 3.91E−06 | −6.91E+04 |
| CD.B/DZ.RGS13.AICDA | B2M | 1.71E−10 | −0.683005891 | 0.936 | 0.969 | 3.94E−06 | −1.73E+05 |
| CD.Mac.APOE.PTGDS | ITM2C | 1.77E−10 | 0.409159935 | 0.252 | 0.143 | 4.08E−06 | 1.00E+05 |
| CD.Endth/Cap.CD36.FLT1 | MTRNR2L12 | 1.77E−10 | 0.844280228 | 0.492 | 0.224 | 4.08E−06 | 2.07E+05 |
| CD.Mac.APOE.PTGDS | CXCR4 | 1.78E−10 | −0.284226896 | 0.317 | 0.476 | 4.10E−06 | −6.94E+04 |
| CD.Plsma/IgA.AL9287683.RPL39 | CDV3 | 1.84E−10 | −0.337547005 | 0.377 | 0.497 | 4.23E−06 | −7.98E+04 |
| CD.Plsma/IgA.IGLC3.IGLC2.2 | MT2A | 1.84E−10 | −0.576106522 | 0.376 | 0.61 | 4.23E−06 | −1.36E+05 |
| CD.Plsma/IgA.IGLL5.IGLV2-14 | RPS24 | 1.90E−10 | 0.31365052 | 0.975 | 0.939 | 4.37E−06 | 7.17E+04 |
| CD.Plsma/IgM.IGHM.JCHAIN | EIF3L | 1.91E−10 | 0.260858397 | 0.542 | 0.376 | 4.40E−06 | 5.93E+04 |
| CD.Plsma/IgA.AL9287683.RPL39 | YPEL3 | 1.91E−10 | −0.265628752 | 0.194 | 0.338 | 4.41E−06 | −6.03E+04 |
| CD.Plsma/IgA.IGHA1.IGLV6-57 | RPL6 | 1.96E−10 | 0.452927264 | 0.957 | 0.947 | 4.52E−06 | 1.00E+05 |
| CD.cDC1.CLEC9A.IDO1 | EEF1A1 | 1.97E−10 | 0.612579588 | 0.933 | 0.878 | 4.53E−06 | 1.35E+05 |
| CD.T.CCR7.GPR183 | REG3A | 1.97E−10 | −0.570359805 | 0.039 | 0.113 | 4.53E−06 | −1.26E+05 |
| CD.Fibro.TFPI2.CCL13 | CLMP | 1.97E−10 | −0.800624154 | 0.207 | 0.538 | 4.53E−06 | −1.77E+05 |
| CD.Mac.APOE.PTGDS | ELF1 | 1.98E−10 | −0.312205432 | 0.276 | 0.416 | 4.57E−06 | −6.84E+04 |
| CD.NK.CCL3.CD160 | NEAT1 | 1.99E−10 | −0.436290686 | 0.48 | 0.572 | 4.58E−06 | −9.53E+04 |
| CD.Plsma/IgM.IGHM.JCHAIN | JUND | 2.01E−10 | 0.275934144 | 0.556 | 0.367 | 4.64E−06 | 5.95E+04 |
| CD.Fibro.TFPI2.CCL13 | EEF1D | 2.02E−10 | 0.367482838 | 0.972 | 0.925 | 4.64E−06 | 7.92E+04 |
| CD.Plsma/IgM.IGHM.JCHAIN | EZR | 2.02E−10 | 0.427297794 | 0.582 | 0.447 | 4.64E−06 | 9.20E+04 |
| CD.B.IGLC3.JCHAIN | HLA-DRB5 | 2.03E−10 | 0.894941038 | 0.671 | 0.238 | 4.67E−06 | 1.92E+05 |
| CD.T.TNFRSF18.FOXP3 | IGKC | 2.05E−10 | 0.679564551 | 0.972 | 0.951 | 4.72E−06 | 1.44E+05 |
| CD.Plsma/IgM.IGHM.JCHAIN | RSRP1 | 2.07E−10 | 0.269772262 | 0.493 | 0.32 | 4.77E−06 | 5.66E+04 |
| CD.Fibro.TFPI2.CCL13 | TOB1 | 2.07E−10 | −0.272697167 | 0.013 | 0.161 | 4.77E−06 | −5.72E+04 |
| CD.Paneth.DEFA6.ITLN2 | IGHV4-39 | 2.08E−10 | 0.257114425 | 0.18 | 0.012 | 4.79E−06 | 5.37E+04 |
| CD.Prolif/EC.UBE2C.CCNB2 | TSPAN8 | 2.09E−10 | −0.492991638 | 0.941 | 0.988 | 4.81E−06 | −1.03E+05 |
| CD.T.CCL20.RORA | RPL37 | 2.13E−10 | 0.294974886 | 0.92 | 0.832 | 4.90E−06 | 6.02E+04 |
| CD.B.MT-CO2.MT-CO1 | CYBA | 2.18E−10 | −0.443469667 | 0.66 | 0.791 | 5.03E−06 | −8.82E+04 |
| CD.T.IFI44L.PTGER4 | RPS4Y1 | 2.22E−10 | −0.989720625 | 0.102 | 0.433 | 5.11E−06 | −1.94E+05 |
| CD.Plsma/IgA.IGHA2.IGKV1-12 | IGHG2 | 2.23E−10 | −0.292161124 | 0.227 | 0.401 | 5.12E−06 | −5.70E+04 |
| CD.Paneth.DEFA6.ITLN2 | IGLC2 | 2.24E−10 | −0.343633961 | 0.883 | 0.63 | 5.15E−06 | −6.67E+04 |
| CD.Mito.MT-CO2.MT-ND2 | MT-ND4L | 2.26E−10 | −0.450238976 | 0.567 | 0.697 | 5.20E−06 | −8.66E+04 |
| CD.Plsma/IgA.IGLC3.IGLC2.2 | DERL3 | 2.27E−10 | −0.396684416 | 0.962 | 0.97 | 5.23E−06 | −7.58E+04 |
| CD.Prolif/EC.UBE2C.CCNB2 | ECI1 | 2.29E−10 | −0.59214977 | 0.255 | 0.715 | 5.27E−06 | −1.12E+05 |
| CD.NK.CCL3.CD160 | MT-ND5 | 2.39E−10 | −0.352010555 | 0.656 | 0.723 | 5.50E−06 | −6.40E+04 |
| CD.Fibro.TFPI2.CCL13 | C9orf72 | 2.41E−10 | −0.40836717 | 0.01 | 0.151 | 5.55E−06 | −7.36E+04 |
| CD.Bclls.CD69.GPR183 | MIF | 2.48E−10 | −0.695458332 | 0.173 | 0.354 | 5.72E−06 | −1.22E+05 |
| CD.Paneth.DEFA6.ITLN2 | CCND2 | 2.49E−10 | 0.360892905 | 0.415 | 0.138 | 5.73E−06 | 6.29E+04 |
| CD.Plsma/IgA.IGLV1-51.IGLV10-54 | MT-ND1 | 2.51E−10 | 0.568122715 | 0.923 | 0.851 | 5.78E−06 | 9.83E+04 |
| CD.Plsma/IgA.IGLV2-14.IGLV3-19 | RPL11 | 2.54E−10 | 0.460430421 | 0.994 | 0.956 | 5.84E−06 | 7.89E+04 |
| CD.Mono.S100A8.S100A9 | PFN1 | 2.55E−10 | −0.447573484 | 0.886 | 0.913 | 5.88E−06 | −7.61E+04 |
| CD.Bclls.CD69.GPR183 | RGCC | 2.56E−10 | 0.610598746 | 0.203 | 0.072 | 5.89E−06 | 1.04E+05 |
| CD.Plsma/IgA.IGLC3.IGLC2.2 | RPL28 | 2.59E−10 | −0.307567785 | 0.995 | 1 | 5.96E−06 | −5.16E+04 |
| CD.Plsma/IgA.IGHA1.IGKC | IGKV3-11 | 2.64E−10 | −0.407702917 | 0.079 | 0.225 | 6.07E−06 | −6.71E+04 |
| CD.Prolif/EC.UBE2C.CCNB2 | EIF5A | 2.72E−10 | −0.396154751 | 0.686 | 0.934 | 6.26E−06 | −6.33E+04 |
| CD.Plsma/IgA.IGHA2.IGKV1-12 | PSME2 | 2.73E−10 | −0.288071328 | 0.549 | 0.675 | 6.30E−06 | −4.58E+04 |
| CD.T.MT-CO2.MT-CO3 | MTRNR2L8 | 2.80E−10 | 0.897142068 | 0.185 | 0.041 | 6.43E−06 | 1.39E+05 |
| CD.Plsma/IgG.IGHGP.IGHG2 | NEAT1 | 2.87E−10 | 0.325166744 | 0.846 | 0.841 | 6.60E−06 | 4.92E+04 |
| CD.Plsma/IgM.IGHM.JCHAIN | TSC22D3 | 2.94E−10 | 0.35682704 | 0.785 | 0.724 | 6.76E−06 | 5.28E+04 |
| CD.Plsma/IgA.IGHA2.IGKV1-12 | NDUFC2 | 2.94E−10 | 0.284284241 | 0.595 | 0.406 | 6.76E−06 | 4.20E+04 |
| CD.Fibro.TFPI2.CCL13 | ATP51 | 2.94E−10 | −0.600210935 | 0.406 | 0.742 | 6.77E−06 | −8.87E+04 |
| CD.Plsma/IgA.IGLC3.IGLC2.2 | RPL13 | 2.98E−10 | −0.253254675 | 1 | 1 | 6.85E−06 | −3.70E+04 |
| CD.DC.CCR7.FSCN1 | IGKC | 3.01E−10 | 0.857222475 | 0.957 | 0.944 | 6.94E−06 | 1.24E+05 |
| CD.Mono.S100A8.S100A9 | RPL30 | 3.02E−10 | 0.637908449 | 0.876 | 0.787 | 6.95E−06 | 9.18E+04 |
| CD.cDC1.CLEC9A.XCR1 | MTRNR2L8 | 3.14E−10 | 0.396444545 | 0.272 | 0.04 | 7.23E−06 | 5.48E+04 |
| CD.Fibro.TFPI2.CCL13 | MIR4458HG | 3.16E−10 | −0.46860967 | 0.065 | 0.301 | 7.27E−06 | −6.45E+04 |
| CD.TA.PLCG2.SOX4 | KRT8 | 3.17E−10 | −0.762421979 | 0.513 | 0.758 | 7.29E−06 | −1.05E+05 |
| CD.Fibro.TFPI2.CCL13 | CCL13 | 3.21E−10 | −0.740740016 | 0.84 | 0.935 | 7.39E−06 | −1.00E+05 |
| CD.Paneth.DEFA6.ITLN2 | SLC38A2 | 3.32E−10 | 0.378141825 | 0.507 | 0.213 | 7.65E−06 | 4.94E+04 |
| CD.Plsma/IgA.IGKV1-12.IGHA2 | RPS4X | 3.33E−10 | 0.440025175 | 0.978 | 1 | 7.66E−06 | 5.74E+04 |
| CD.Plsma/IgA.IGLL5.IGLV2-14 | ERLEC1 | 3.34E−10 | −0.39234893 | 0.611 | 0.78 | 7.68E−06 | −5.11E+04 |
| CD.Fibro.TFPI2.CCL13 | LINC00116 | 3.35E−10 | −0.388699095 | 0.075 | 0.323 | 7.72E−06 | −5.04E+04 |
| CD.B.MT-CO2.MT-CO1 | MT-ATP6 | 3.37E−10 | 0.2800237 | 0.976 | 0.958 | 7.75E−06 | 3.61E+04 |
| CD.B.MT-CO2.MT-CO1 | RPS5 | 3.38E−10 | −0.277931438 | 0.895 | 0.946 | 7.79E−06 | −3.57E+04 |
| CD.Fibro.TFPI2.CCL13 | IGLC2 | 3.43E−10 | 0.465054957 | 0.891 | 0.796 | 7.88E−06 | 5.90E+04 |
| CD.Fibro.TFPI2.CCL13 | DPT | 3.44E−10 | −0.888336404 | 0.24 | 0.57 | 7.91E−06 | −1.12E+05 |
| CD.B.IGHG1.IGLC2 | ARHGAP24 | 3.47E−10 | −0.824770338 | 0.059 | 0.224 | 7.98E−06 | −1.03E+05 |
| CD.Mstcl.CTSG.CD69 | MYADM | 3.49E−10 | 0.887558236 | 0.368 | 0.137 | 8.04E−06 | 1.10E+05 |
| CD.NK.CCL3.CD160 | RUNX3 | 3.55E−10 | 0.473232721 | 0.254 | 0.135 | 8.17E−06 | 5.79E+04 |
| CD.Plsma/IgA.MT-ND5.MT-ND4L | APOA4 | 3.61E−10 | 0.337572745 | 0.116 | 0 | 8.30E−06 | 4.06E+04 |
| CD.Fibro.TFPI2.CCL13 | IL15RA | 3.68E−10 | −0.491504339 | 0.072 | 0.312 | 8.46E−06 | −5.81E+04 |
| CD.Plsma/IgM.IGHM.JCHAIN | NPM1 | 3.74E−10 | 0.277858984 | 0.785 | 0.669 | 8.62E−06 | 3.22E+04 |
| CD.Plsma/IgA.IGKV1-12.IGHA2 | RPS3 | 3.83E−10 | 0.498573922 | 0.964 | 0.932 | 8.83E−06 | 5.65E+04 |
| CD.Mac.APOE.PTGDS | SRRM2 | 3.84E−10 | −0.287025612 | 0.21 | 0.34 | 8.84E−06 | −3.25E+04 |
| CD.Plsma/IgA.IGLV2-14.IGLV3-19 | HINT1 | 3.86E−10 | 0.604993811 | 0.886 | 0.603 | 8.89E−06 | 6.80E+04 |
| CD.cDC1.CLEC9A.IDO1 | HLA-DRB5 | 3.95E−10 | 0.859413101 | 0.656 | 0.244 | 9.10E−06 | 9.44E+04 |
| CD.Plsma/IgA.AL9287683.RPL39 | RNASET2 | 3.96E−10 | −0.31714472 | 0.593 | 0.644 | 9.12E−06 | −3.48E+04 |
| CD.cDC1.CLEC9A.XCR1 | JUN | 4.00E−10 | 0.704579542 | 0.705 | 0.414 | 9.22E−06 | 7.64E+04 |
| CD.Mac.SEPP1.CXCL3 | HLA-DRB5 | 4.05E−10 | 1.60789037 | 0.817 | 0.321 | 9.33E−06 | 1.72E+05 |
| CD.Plsma/IgA.IGLV2-14.IGLV3-19 | RPS15A | 4.11E−10 | 0.513800163 | 0.988 | 0.971 | 9.47E−06 | 5.43E+04 |
| CD.Plsma/IgA.IGHA1.IGKC | DDX3X | 4.19E−10 | −0.255079269 | 0.209 | 0.362 | 9.65E−06 | −2.64E+04 |
| CD.Prolif/EC.UBE2C.CCNB2 | RPS2 | 4.20E−10 | −0.326325129 | 0.98 | 0.996 | 9.67E−06 | −3.37E+04 |
| CD.Fibro.RERGL.NTRK2 | IGHG1 | 4.22E−10 | 1.392394334 | 0.508 | 0.068 | 9.71E−06 | 1.43E+05 |
| CD.TA.PLCG2.SOX4 | IGKC | 4.23E−10 | 0.56166739 | 0.994 | 0.941 | 9.74E−06 | 5.77E+04 |

TABLE 2B
Selected Genes for subsets having differentially expressed genes between FR and PR
(Positive direction is enriched in FR and negative direction is enriched in PR).

| subset | gene | p_val | avg_logFC | pct.1 | pct.2 | p_val_adj | STAT |
|---|---|---|---|---|---|---|---|
| CD.NK.CCL3.CD160 | IFITM1 | 2.44E−45 | 0.762641682 | 0.546 | 0.203 | 5.62E−41 | 1.36E+40 |
| CD.Paneth.DEFA6.ITLN2 | APOA1 | 1.28E−38 | 1.290328567 | 0.532 | 0.004 | 2.95E−34 | 4.37E+33 |
| CD.Fibro.TFPI2.CCL13 | TPT1 | 7.06E−38 | 0.942527275 | 1 | 0.978 | 1.62E−33 | 5.80E+32 |
| CD.Paneth.DEFA6.ITLN2 | FABP6 | 1.18E−35 | 1.059253389 | 0.512 | 0.008 | 2.73E−31 | 3.88E+30 |
| CD.Fibro.TFPI2.CCL13 | NACA | 1.36E−30 | 1.030564111 | 0.959 | 0.796 | 3.12E−26 | 3.30E+25 |
| CD.Paneth.DEFA6.ITLN2 | APOA4 | 2.78E−30 | 0.94821013 | 0.424 | 0 | 6.40E−26 | 1.48E+25 |
| CD.Paneth.DEFA6.ITLN2 | MIF | 1.21E−26 | 0.835744974 | 0.634 | 0.157 | 2.79E−22 | 3.00E+21 |
| CD.NK.CCL3.CD160 | HOPX | 2.96E−20 | 0.524031331 | 0.472 | 0.25 | 6.81E−16 | 7.70E+14 |
| CD.Paneth.DEFA6.ITLN2 | SPINK4 | 5.63E−19 | 0.903869436 | 0.58 | 0.169 | 1.30E−14 | 6.98E+13 |
| CD.T.CCL20.RORA | HOPX | 1.03E−16 | 0.748859499 | 0.335 | 0.12 | 2.38E−12 | 3.15E+11 |
| CD.NK.CCL3.CD160 | CMC1 | 2.34E−30 | −0.677153946 | 0.582 | 0.74 | 5.39E−26 | −1.26E+25 |
| CD.Fibro.TFPI2.CCL13 | TNFRSF11B | 4.33E−29 | −1.732694376 | 0.065 | 0.516 | 9.98E−25 | −1.74E+24 |
| CD.Mac.APOE.PTGDS | BRI3 | 2.29E−29 | −0.520918977 | 0.353 | 0.581 | 5.27E−25 | −9.88E+23 |
| CD.Fibro.TFPI2.CCL13 | COL1A2 | 6.05E−27 | −0.90539105 | 0.853 | 0.978 | 1.39E−22 | −6.50E+21 |
| CD.NK.CCL3.CD160 | NKG7 | 2.41E−26 | −0.32745046 | 0.919 | 0.946 | 5.55E−22 | −5.90E+20 |
| CD.Mac.APOE.PTGDS | APOE | 3.63E−25 | −0.42814411 | 0.724 | 0.911 | 8.35E−21 | −5.12E+19 |
| CD.Fibro.TFPI2.CCL13 | TFPI2 | 5.03E−24 | −1.680266588 | 0.419 | 0.817 | 1.16E−19 | −1.45E+19 |
| CD.NK.CCL3.CD160 | AREG | 1.62E−23 | −0.638267424 | 0.559 | 0.701 | 3.72E−19 | −1.71E+18 |
| CD.NK.CCL3.CD160 | KLRC1 | 5.52E−21 | −0.606558945 | 0.267 | 0.472 | 1.27E−16 | −4.78E+15 |
| CD.Fibro.TFPI2.CCL13 | HTRA3 | 3.22E−19 | −0.730188163 | 0.09 | 0.484 | 7.41E−15 | −9.86E+13 |
| CD.Fibro.TFPI2.CCL13 | COL1A1 | 4.98E−19 | −1.036024325 | 0.266 | 0.731 | 1.15E−14 | −9.03E+13 |
| CD.Fibro.TFPI2.CCL13 | HIF1A | 2.38E−18 | −0.977155958 | 0.194 | 0.645 | 5.47E−14 | −1.79E+13 |
| CD.Mac.APOE.PTGDS | STAT1 | 1.76E−18 | −0.276109272 | 0.481 | 0.702 | 4.04E−14 | −6.83E+12 |
| CD.Fibro.TFPI2.CCL13 | SLC16A4 | 6.21E−18 | −0.754569078 | 0.078 | 0.441 | 1.43E−13 | −5.28E+12 |
| CD.Fibro.TFPI2.CCL13 | SERPINE2 | 3.72E−17 | −1.269469223 | 0.326 | 0.72 | 8.56E−13 | −1.48E+12 |
| CD.Fibro.TFPI2.CCL13 | CCL11 | 7.88E−17 | −0.865291325 | 0.938 | 0.978 | 1.81E−12 | −4.77E+11 |
| CD.Mac.APOE.PTGDS | SAMHD1 | 6.48E−17 | −0.308775283 | 0.374 | 0.576 | 1.49E−12 | −2.07E+11 |
| CD.Mac.APOE.PTGDS | TAX1BP1 | 8.74E−17 | −0.252468756 | 0.39 | 0.576 | 2.01E−12 | −1.26E+11 |
| CD.Mac.APOE.PTGDS | TXN | 1.52E−16 | −0.417758346 | 0.535 | 0.708 | 3.49E−12 | −1.20E+11 |
| CD.Mac.APOE.PTGDS | GPR65 | 2.83E−16 | −0.340836873 | 0.156 | 0.325 | 6.52E−12 | −5.23E+10 |
| CD.Fibro.TFPI2.CCL13 | CEBPB | 9.16E−16 | −0.806394045 | 0.116 | 0.473 | 2.11E−11 | −3.82E+10 |
| CD.Fibro.TFPI2.CCL13 | GSN | 3.69E−15 | −1.060184806 | 0.682 | 0.871 | 8.49E−11 | −1.25E+10 |
| CD.Fibro.TFPI2.CCL13 | EMILIN1 | 3.23E−15 | −0.83359965 | 0.406 | 0.774 | 7.42E−11 | −1.12E+10 |
| CD.Fibro.TFPI2.CCL13 | CTNNB1 | 5.00E−15 | −0.8790437 | 0.114 | 0.473 | 1.15E−10 | −7.63E+09 |
| CD.Fibro.TFPI2.CCL13 | COL4A1 | 3.64E−15 | −0.636207184 | 0.098 | 0.452 | 8.37E−11 | −7.60E+09 |
| CD.Mac.APOE.PTGDS | CLEC12A | 1.95E−15 | −0.301631062 | 0.12 | 0.281 | 4.48E−11 | −6.73E+09 |
| CD.Fibro.TFPI2.CCL13 | PTGER4 | 5.25E−15 | −0.59979123 | 0.119 | 0.495 | 1.21E−10 | −4.97E+09 |
| CD.Fibro.TFPI2.CCL13 | BDKRB1 | 6.61E−15 | −0.420997685 | 0.031 | 0.28 | 1.52E−10 | −2.77E+09 |
| CD.Fibro.TFPI2.CCL13 | SKIL | 1.13E−14 | −0.624732136 | 0.062 | 0.355 | 2.59E−10 | −2.41E+09 |
| CD.NK.CCL3.CD160 | PFN1 | 7.52E−15 | −0.257625736 | 0.903 | 0.915 | 1.73E−10 | −1.49E+09 |

TABLE 2C
Genes differentially expressed between FR and PR for CD.NK.CCL3.CD160
(CD.Tclls.cytotoxic_IEL_FCER1G_NKG7_TYROBP_CD160_AREG)
and CD.Mac.APOE.PTGDS (CD.Mloid.macrophage_APOE_C1Q_CD63_{—}
CD14_AXL) subsets (Positive direction is enriched in FR and
negative direction is enriched in PR).

| subset | gene | p_val | avg_logFC | pct.1 | pct.2 | p_val_adj | STAT |
|---|---|---|---|---|---|---|---|
| CD.Mac.APOE.PTGDS | HLA-DRB5 | 1.17E−42 | 0.890716089 | 0.668 | 0.461 | 2.69E−38 | 3.31E+37 |
| CD.Mac.APOE.PTGDS | MTRNR2L8 | 2.40E−18 | 0.550899657 | 0.172 | 0.055 | 5.52E−14 | 9.98E+12 |
| CD.Mac.APOE.PTGDS | RNASE1 | 5.24E−18 | 0.943671019 | 0.106 | 0.02 | 1.21E−13 | 7.83E+12 |
| CD.Mac.APOE.PTGDS | SEPP1 | 1.59E−17 | 0.351604076 | 0.852 | 0.731 | 3.67E−13 | 9.58E+11 |
| CD.Mac.APOE.PTGDS | C1QC | 2.86E−17 | 0.321505807 | 0.898 | 0.915 | 6.57E−13 | 4.89E+11 |
| CD.Mac.APOE.PTGDS | ALOX5AP | 9.30E−17 | 0.372598209 | 0.14 | 0.039 | 2.14E−12 | 1.74E+11 |
| CD.Mac.APOE.PTGDS | RPL41 | 3.23E−16 | 0.261109077 | 0.93 | 0.942 | 7.43E−12 | 3.52E+10 |
| CD.Mac.APOE.PTGDS | MTRNR2L12 | 9.35E−16 | 0.535587992 | 0.187 | 0.072 | 2.15E−11 | 2.49E+10 |
| CD.Mac.APOE.PTGDS | RPL39 | 1.41E−15 | 0.255731716 | 0.889 | 0.914 | 3.25E−11 | 7.86E+09 |
| CD.Mac.APOE.PTGDS | IGKC | 9.33E−15 | 0.649813562 | 0.966 | 0.955 | 2.15E−10 | 3.03E+09 |
| CD.Mac.APOE.PTGDS | RPS28 | 9.48E−15 | 0.279284835 | 0.888 | 0.897 | 2.18E−10 | 1.28E+09 |
| CD.Mac.APOE.PTGDS | CLEC9A | 2.20E−14 | 0.254871623 | 0.107 | 0.027 | 5.06E−10 | 5.04E+08 |
| CD.Mac.APOE.PTGDS | MT-ND3 | 5.65E−14 | 0.419805572 | 0.771 | 0.805 | 1.30E−09 | 3.23E+08 |
| CD.Mac.APOE.PTGDS | CXCL12 | 8.83E−14 | 0.465772988 | 0.283 | 0.148 | 2.03E−09 | 2.29E+08 |
| CD.Mac.APOE.PTGDS | RPS26 | 1.07E−12 | 0.428897685 | 0.693 | 0.672 | 2.46E−08 | 1.74E+07 |
| CD.Mac.APOE.PTGDS | IGHA1 | 1.51E−12 | 0.295732867 | 0.901 | 0.872 | 3.48E−08 | 8.49E+06 |
| CD.Mac.APOE.PTGDS | TCOF1 | 2.62E−11 | 0.282441617 | 0.159 | 0.068 | 6.03E−07 | 4.69E+05 |
| CD.Mac.APOE.PTGDS | ITM2C | 1.77E−10 | 0.409159935 | 0.252 | 0.143 | 4.08E−06 | 1.00E+05 |
| CD.Mac.APOE.PTGDS | TPT1 | 6.12E−10 | 0.360482382 | 0.917 | 0.917 | 1.41E−05 | 2.56E+04 |
| CD.Mac.APOE.PTGDS | DNASE1L3 | 4.77E−09 | 0.464946377 | 0.642 | 0.597 | 0.000109889 | 4.23E+03 |
| CD.Mac.APOE.PTGDS | LGMN | 6.00E−09 | 0.477055281 | 0.602 | 0.537 | 0.000138075 | 3.46E+03 |
| CD.Mac.APOE.PTGDS | IGLC2 | 1.26E−08 | 0.779968007 | 0.649 | 0.618 | 0.000289627 | 2.69E+03 |
| CD.Mac.APOE.PTGDS | CD163L1 | 1.45E−08 | 0.380177819 | 0.202 | 0.112 | 0.000334001 | 1.14E+03 |
| CD.Mac.APOE.PTGDS | RPL31 | 2.25E−07 | 0.298712858 | 0.767 | 0.779 | 0.005184281 | 5.76E+01 |
| CD.Mac.APOE.PTGDS | MARCKSL1 | 2.50E−07 | 0.312743203 | 0.169 | 0.093 | 0.005749741 | 5.44E+01 |
| CD.Mac.APOE.PTGDS | GATM | 2.90E−07 | 0.323625345 | 0.262 | 0.167 | 0.006670687 | 4.85E+01 |
| CD.Mac.APOE.PTGDS | ZFP36 | 7.76E−07 | 0.435645786 | 0.462 | 0.417 | 0.017870824 | 2.44E+01 |
| CD.Mac.APOE.PTGDS | CFD | 1.11E−06 | 0.487649757 | 0.561 | 0.526 | 0.025535828 | 1.91E+01 |
| CD.Mac.APOE.PTGDS | RPL38 | 1.50E−06 | 0.279000264 | 0.724 | 0.772 | 0.034511194 | 8.08E+00 |
| CD.Mac.APOE.PTGDS | A2M | 2.16E−06 | 0.370788997 | 0.327 | 0.236 | 0.049645522 | 7.47E+00 |
| CD.Mac.APOE.PTGDS | CXCL3 | 5.48E−06 | 0.532509427 | 0.296 | 0.219 | 0.126161271 | 4.22E+00 |
| CD.Mac.APOE.PTGDS | ITGB7 | 1.61E−05 | 0.362974361 | 0.182 | 0.118 | 0.371371348 | 9.77E−01 |
| CD.Mac.APOE.PTGDS | IGHG1 | 0.000226117 | 0.798130485 | 0.268 | 0.216 | 1 | 7.98E−01 |
| CD.Mac.APOE.PTGDS | LINC00996 | 2.01E−05 | 0.308207976 | 0.244 | 0.169 | 0.462322107 | 6.67E−01 |
| CD.Mac.APOE.PTGDS | NFKBIA | 2.67E−05 | 0.401386108 | 0.623 | 0.683 | 0.615004733 | 6.53E−01 |
| CD.Mac.APOE.PTGDS | IL1B | 0.031405004 | 0.445518673 | 0.465 | 0.491 | 1 | 4.46E−01 |
| CD.Mac.APOE.PTGDS | CXCL1 | 0.10932606 | 0.398665712 | 0.127 | 0.106 | 1 | 3.99E−01 |
| CD.Mac.APOE.PTGDS | CCL3L3 | 0.032168652 | 0.374669848 | 0.405 | 0.379 | 1 | 3.75E−01 |
| CD.Mac.APOE.PTGDS | FOLR2 | 0.000497015 | 0.369508886 | 0.366 | 0.306 | 1 | 3.70E−01 |
| CD.Mac.APOE.PTGDS | RPS10 | 0.001962253 | 0.365827279 | 0.564 | 0.59 | 1 | 3.66E−01 |
| CD.Mac.APOE.PTGDS | STAB1 | 0.007474274 | 0.358889716 | 0.317 | 0.269 | 1 | 3.59E−01 |
| CD.Mac.APOE.PTGDS | AXL | 0.017792784 | 0.348504942 | 0.369 | 0.344 | 1 | 3.49E−01 |
| CD.Mac.APOE.PTGDS | EBI3 | 0.000200234 | 0.328559628 | 0.192 | 0.133 | 1 | 3.29E−01 |
| CD.Mac.APOE.PTGDS | FCGR2A | 0.000356179 | 0.327439032 | 0.333 | 0.269 | 1 | 3.27E−01 |
| CD.Mac.APOE.PTGDS | TGFBI | 0.010474517 | 0.321540062 | 0.426 | 0.405 | 1 | 3.22E−01 |
| CD.Mac.APOE.PTGDS | CCL2 | 0.00083395 | 0.316288485 | 0.127 | 0.08 | 1 | 3.16E−01 |
| CD.Mac.APOE.PTGDS | YWHAE | 0.040751942 | 0.313924529 | 0.442 | 0.419 | 1 | 3.14E−01 |
| CD.Mac.APOE.PTGDS | C1orf56 | 0.005061187 | 0.306665994 | 0.21 | 0.164 | 1 | 3.07E−01 |
| CD.Mac.APOE.PTGDS | VSIG4 | 0.145693566 | 0.306077669 | 0.39 | 0.394 | 1 | 3.06E−01 |
| CD.Mac.APOE.PTGDS | CNBP | 0.093602026 | 0.299736106 | 0.317 | 0.289 | 1 | 3.00E−01 |
| CD.Mac.APOE.PTGDS | POLR1D | 0.503800436 | 0.298785819 | 0.228 | 0.219 | 1 | 2.99E−01 |
| CD.Mac.APOE.PTGDS | SLCO2B1 | 0.000372188 | 0.298360233 | 0.345 | 0.276 | 1 | 2.98E−01 |
| CD.Mac.APOE.PTGDS | NCF4 | 0.176466827 | 0.296462713 | 0.34 | 0.329 | 1 | 2.96E−01 |
| CD.Mac.APOE.PTGDS | S100A9 | 0.191580967 | 0.284374405 | 0.106 | 0.131 | 1 | 2.84E−01 |
| CD.Mac.APOE.PTGDS | TMEM37 | 0.274583645 | 0.280948152 | 0.146 | 0.13 | 1 | 2.81E−01 |
| CD.Mac.APOE.PTGDS | SEPT15 | 0.680977578 | 0.278524288 | 0.372 | 0.379 | 1 | 2.79E−01 |
| CD.Mac.APOE.PTGDS | UQCRB | 0.010846007 | 0.278148341 | 0.66 | 0.692 | 1 | 2.78E−01 |
| CD.Mac.APOE.PTGDS | TIMM10 | 0.120241013 | 0.27746283 | 0.176 | 0.149 | 1 | 2.77E−01 |
| CD.Mac.APOE.PTGDS | ADORA3 | 0.001710206 | 0.275842037 | 0.151 | 0.106 | 1 | 2.76E−01 |
| CD.Mac.APOE.PTGDS | NFKBIZ | 0.001240261 | 0.273557201 | 0.312 | 0.258 | 1 | 2.74E−01 |
| CD.Mac.APOE.PTGDS | HSPA1A | 0.520003784 | 0.273177164 | 0.446 | 0.519 | 1 | 2.73E−01 |
| CD.Mac.APOE.PTGDS | RPL36A | 0.046114999 | 0.271977251 | 0.55 | 0.584 | 1 | 2.72E−01 |
| CD.Mac.APOE.PTGDS | CCL4L2 | 0.00735259 | 0.271939311 | 0.421 | 0.369 | 1 | 2.72E−01 |
| CD.Mac.APOE.PTGDS | GADD45B | 0.175815903 | 0.270734747 | 0.462 | 0.505 | 1 | 2.71E−01 |
| CD.Mac.APOE.PTGDS | SAP18 | 0.849519066 | 0.269907191 | 0.431 | 0.46 | 1 | 2.70E−01 |
| CD.Mac.APOE.PTGDS | PABPC1 | 0.070125902 | 0.267075585 | 0.634 | 0.753 | 1 | 2.67E−01 |
| CD.Mac.APOE.PTGDS | LIPA | 0.005353675 | 0.265975493 | 0.691 | 0.737 | 1 | 2.66E−01 |
| CD.Mac.APOE.PTGDS | JCHAIN | 0.000351549 | 0.265700281 | 0.694 | 0.667 | 1 | 2.66E−01 |
| CD.Mac.APOE.PTGDS | IER3 | 0.006791196 | 0.264332476 | 0.421 | 0.38 | 1 | 2.64E−01 |
| CD.Mac.APOE.PTGDS | MYADM | 0.000221458 | 0.262434013 | 0.198 | 0.137 | 1 | 2.62E−01 |
| CD.Mac.APOE.PTGDS | PABPC4 | 0.257905576 | 0.254793549 | 0.288 | 0.277 | 1 | 2.55E−01 |
| CD.Mac.APOE.PTGDS | CBX6 | 0.000108274 | 0.25259034 | 0.202 | 0.136 | 1 | 2.53E−01 |
| CD.Mac.APOE.PTGDS | LPAR6 | 0.092099216 | 0.250491435 | 0.345 | 0.323 | 1 | 2.50E−01 |
| CD.Mac.APOE.PTGDS | FBP1 | 0.000295896 | −0.281594033 | 0.12 | 0.181 | 1 | −2.82E−01 |
| CD.Mac.APOE.PTGDS | IGLC3 | 2.46E−06 | −0.273158897 | 0.32 | 0.445 | 0.05671008 | −4.82E+00 |
| CD.Mac.APOE.PTGDS | TMEM141 | 2.36E−06 | −0.269885104 | 0.086 | 0.161 | 0.054359531 | −4.96E+00 |
| CD.Mac.APOE.PTGDS | MRPL20 | 8.68E−07 | −0.263904194 | 0.242 | 0.336 | 0.019978337 | −1.32E+01 |
| CD.Mac.APOE.PTGDS | TWISTNB | 8.77E−07 | −0.299520212 | 0.169 | 0.263 | 0.020185545 | −1.48E+01 |
| CD.Mac.APOE.PTGDS | ITPR2 | 5.31E−07 | −0.253981823 | 0.242 | 0.348 | 0.012220777 | −2.08E+01 |
| CD.Mac.APOE.PTGDS | VMO1 | 4.78E−07 | −0.301086091 | 0.184 | 0.283 | 0.011003536 | −2.74E+01 |
| CD.Mac.APOE.PTGDS | IQGAP1 | 3.95E−07 | −0.274761891 | 0.249 | 0.353 | 0.009099895 | −3.02E+01 |
| CD.Mac.APOE.PTGDS | GABARAP | 3.31E−07 | −0.252547969 | 0.076 | 0.157 | 0.007608528 | −3.32E+01 |
| CD.Mac.APOE.PTGDS | CHMP4B | 2.06E−07 | −0.264556191 | 0.192 | 0.297 | 0.004745139 | −5.58E+01 |
| CD.Mac.APOE.PTGDS | CYP27A1 | 2.38E−07 | −0.309387345 | 0.115 | 0.21 | 0.00548586 | −5.64E+01 |
| CD.Mac.APOE.PTGDS | CEBPD | 1.70E−07 | −0.266902809 | 0.268 | 0.386 | 0.003912362 | −6.82E+01 |
| CD.Mac.APOE.PTGDS | C15orf48 | 1.02E−07 | −0.306247305 | 0.27 | 0.392 | 0.002349105 | −1.30E+02 |
| CD.Mac.APOE.PTGDS | ARL4A | 2.02E−08 | −0.316716223 | 0.223 | 0.345 | 0.00046417 | −6.82E+02 |
| CD.Mac.APOE.PTGDS | ITGB2 | 9.34E−09 | −0.253657896 | 0.47 | 0.598 | 0.000215031 | −1.18E+03 |
| CD.Mac.APOE.PTGDS | HNRNPU-AS1 | 8.85E−09 | −0.29533976 | 0.091 | 0.189 | 0.000203627 | −1.45E+03 |
| CD.Mac.APOE.PTGDS | TIMP1 | 6.54E−09 | −0.272193996 | 0.426 | 0.576 | 0.000150496 | −1.81E+03 |
| CD.Mac.APOE.PTGDS | YBX3 | 5.52E−09 | −0.250317099 | 0.317 | 0.457 | 0.000127007 | −1.97E+03 |
| CD.Mac.APOE.PTGDS | S100A4 | 2.84E−09 | −0.351195607 | 0.132 | 0.247 | 6.55E−05 | −5.36E+03 |
| CD.Mac.APOE.PTGDS | GBP4 | 1.39E−09 | −0.292973462 | 0.231 | 0.361 | 3.20E−05 | −9.15E+03 |
| CD.Mac.APOE.PTGDS | DNAJC5B | 8.51E−10 | −0.26202818 | 0.093 | 0.201 | 1.96E−05 | −1.34E+04 |
| CD.Mac.APOE.PTGDS | IFNAR1 | 6.06E−10 | −0.271468763 | 0.177 | 0.303 | 1.39E−05 | −1.95E+04 |
| CD.Mac.APOE.PTGDS | ZEB2 | 5.66E−10 | −0.301511995 | 0.307 | 0.447 | 1.30E−05 | −2.31E+04 |
| CD.Mac.APOE.PTGDS | SRRM2 | 3.84E−10 | −0.287025612 | 0.21 | 0.34 | 8.84E−06 | −3.25E+04 |
| CD.Mac.APOE.PTGDS | ELF1 | 1.98E−10 | −0.312205432 | 0.276 | 0.416 | 4.57E−06 | −6.84E+04 |
| CD.Mac.APOE.PTGDS | CXCR4 | 1.78E−10 | −0.284226896 | 0.317 | 0.476 | 4.10E−06 | −6.94E+04 |
| CD.Mac.APOE.PTGDS | YPEL5 | 1.21E−10 | −0.26116878 | 0.311 | 0.46 | 2.80E−06 | −9.34E+04 |
| CD.Mac.APOE.PTGDS | ISG15 | 1.01E−10 | −0.282359502 | 0.312 | 0.466 | 2.32E−06 | −1.22E+05 |
| CD.Mac.APOE.PTGDS | ZNF331 | 6.50E−11 | −0.279037024 | 0.398 | 0.564 | 1.50E−06 | −1.86E+05 |
| CD.Mac.APOE.PTGDS | RSL1D1 | 2.59E−11 | −0.252816686 | 0.247 | 0.388 | 5.96E−07 | −4.24E+05 |
| CD.Mac.APOE.PTGDS | PLEKHO1 | 3.41E−11 | −0.355413976 | 0.263 | 0.403 | 7.86E−07 | −4.52E+05 |
| CD.Mac.APOE.PTGDS | AREG | 3.49E−11 | −0.414915229 | 0.13 | 0.263 | 8.03E−07 | −5.17E+05 |
| CD.Mac.APOE.PTGDS | IGHV3-23 | 1.55E−11 | −0.255707453 | 0.016 | 0.101 | 3.57E−07 | −7.17E+05 |
| CD.Mac.APOE.PTGDS | TFRC | 1.68E−11 | −0.305741329 | 0.306 | 0.461 | 3.88E−07 | −7.88E+05 |
| CD.Mac.APOE.PTGDS | SGK1 | 1.17E−11 | −0.312039954 | 0.408 | 0.57 | 2.69E−07 | −1.16E+06 |
| CD.Mac.APOE.PTGDS | REG1A | 7.73E−12 | −0.398327006 | 0.111 | 0.248 | 1.78E−07 | −2.24E+06 |
| CD.Mac.APOE.PTGDS | MT-CO2 | 1.94E−12 | −0.254916393 | 0.854 | 0.892 | 4.48E−08 | −5.70E+06 |
| CD.Mac.APOE.PTGDS | PLIN2 | 2.37E−12 | −0.338295902 | 0.306 | 0.474 | 5.47E−08 | −6.19E+06 |
| CD.Mac.APOE.PTGDS | CYCS | 1.12E−12 | −0.272600086 | 0.42 | 0.597 | 2.57E−08 | −1.06E+07 |
| CD.Mac.APOE.PTGDS | CD72 | 9.68E−13 | −0.262332377 | 0.127 | 0.267 | 2.23E−08 | −1.18E+07 |
| CD.Mac.APOE.PTGDS | DNAJC15 | 2.90E−13 | −0.262424799 | 0.228 | 0.386 | 6.68E−09 | −3.93E+07 |
| CD.Mac.APOE.PTGDS | FUOM | 3.10E−14 | −0.340357369 | 0.205 | 0.37 | 7.13E−10 | −4.78E+08 |
| CD.Mac.APOE.PTGDS | CFP | 2.11E−14 | −0.348338924 | 0.114 | 0.26 | 4.85E−10 | −7.18E+08 |
| CD.Mac.APOE.PTGDS | ACP5 | 2.52E−14 | −0.480368003 | 0.613 | 0.735 | 5.79E−10 | −8.29E+08 |
| CD.Mac.APOE.PTGDS | SH3BGRL3 | 8.15E−15 | −0.271794939 | 0.595 | 0.754 | 1.88E−10 | −1.45E+09 |
| CD.Mac.APOE.PTGDS | CLEC12A | 1.95E−15 | −0.301631062 | 0.12 | 0.281 | 4.48E−11 | −6.73E+09 |
| CD.Mac.APOE.PTGDS | RGS2 | 5.72E−16 | −0.321799018 | 0.485 | 0.7 | 1.32E−11 | −2.44E+10 |
| CD.Mac.APOE.PTGDS | TLN1 | 5.47E−16 | −0.343921834 | 0.249 | 0.419 | 1.26E−11 | −2.73E+10 |
| CD.Mac.APOE.PTGDS | GPR65 | 2.83E−16 | −0.340836873 | 0.156 | 0.325 | 6.52E−12 | −5.23E+10 |
| CD.Mac.APOE.PTGDS | EFHD2 | 2.30E−16 | −0.426668468 | 0.268 | 0.444 | 5.30E−12 | −8.06E+10 |
| CD.Mac.APOE.PTGDS | TXN | 1.52E−16 | −0.417758346 | 0.535 | 0.708 | 3.49E−12 | −1.20E+11 |
| CD.Mac.APOE.PTGDS | TAX1BP1 | 8.74E−17 | −0.252468756 | 0.39 | 0.576 | 2.01E−12 | −1.26E+11 |
| CD.Mac.APOE.PTGDS | BASP1 | 7.85E−17 | −0.286699685 | 0.115 | 0.292 | 1.81E−12 | −1.59E+11 |
| CD.Mac.APOE.PTGDS | SAMHD1 | 6.48E−17 | −0.308775283 | 0.374 | 0.576 | 1.49E−12 | −2.07E+11 |
| CD.Mac.APOE.PTGDS | MT-ND4L | 5.46E−17 | −0.447027936 | 0.392 | 0.57 | 1.26E−12 | −3.56E+11 |
| CD.Mac.APOE.PTGDS | VAMP5 | 3.58E−18 | −0.311180325 | 0.434 | 0.635 | 8.23E−14 | −3.78E+12 |
| CD.Mac.APOE.PTGDS | STAT1 | 1.76E−18 | −0.276109272 | 0.481 | 0.702 | 4.04E−14 | −6.83E+12 |
| CD.Mac.APOE.PTGDS | APOC1 | 3.96E−20 | −0.381657304 | 0.589 | 0.813 | 9.11E−16 | −4.19E+14 |
| CD.Mac.APOE.PTGDS | NEAT1 | 5.98E−21 | −0.419547338 | 0.48 | 0.691 | 1.38E−16 | −3.05E+15 |
| CD.Mac.APOE.PTGDS | IGSF6 | 3.02E−21 | −0.429516235 | 0.613 | 0.764 | 6.96E−17 | −6.17E+15 |
| CD.Mac.APOE.PTGDS | MALAT1 | 1.17E−21 | −0.373698203 | 0.795 | 0.893 | 2.70E−17 | −1.38E+16 |
| CD.Mac.APOE.PTGDS | IFI30 | 8.42E−22 | −0.460310871 | 0.306 | 0.52 | 1.94E−17 | −2.37E+16 |
| CD.Mac.APOE.PTGDS | MT-ND5 | 1.34E−23 | −0.401192169 | 0.593 | 0.773 | 3.09E−19 | −1.30E+18 |
| CD.Mac.APOE.PTGDS | CTSZ | 1.06E−23 | −0.524056799 | 0.509 | 0.712 | 2.44E−19 | −2.15E+18 |
| CD.Mac.APOE.PTGDS | APOE | 3.63E−25 | −0.42814411 | 0.724 | 0.911 | 8.35E−21 | −5.12E+19 |
| CD.Mac.APOE.PTGDS | CD52 | 4.80E−26 | −0.614487533 | 0.167 | 0.401 | 1.10E−21 | −5.57E+20 |
| CD.Mac.APOE.PTGDS | PTPRC | 8.35E−27 | −0.49336668 | 0.473 | 0.7 | 1.92E−22 | −2.57E+21 |
| CD.Mac.APOE.PTGDS | BRI3 | 2.29E−29 | −0.520918977 | 0.353 | 0.581 | 5.27E−25 | −9.88E+23 |
| CD.NK.CCL3.CD160 | MALAT1 | 9.75E−88 | 0.488061252 | 1 | 0.998 | 2.24E−83 | 2.17E+82 |
| CD.NK.CCL3.CD160 | TPT1 | 5.52E−71 | 0.777663147 | 0.949 | 0.851 | 1.27E−66 | 6.12E+65 |
| CD.NK.CCL3.CD160 | IGKC | 1.54E−66 | 0.762428673 | 0.976 | 0.877 | 3.55E−62 | 2.15E+61 |
| CD.NK.CCL3.CD160 | IGHA1 | 1.11E−64 | 0.668760475 | 0.952 | 0.788 | 2.55E−60 | 2.63E+59 |
| CD.NK.CCL3.CD160 | EEF1A1 | 8.42E−49 | 0.50527135 | 0.964 | 0.93 | 1.94E−44 | 2.61E+43 |
| CD.NK.CCL3.CD160 | IFITM1 | 2.44E−45 | 0.762641682 | 0.546 | 0.203 | 5.62E−41 | 1.36E+40 |
| CD.NK.CCL3.CD160 | IGHM | 1.19E−43 | 0.767968229 | 0.597 | 0.272 | 2.73E−39 | 2.81E+38 |
| CD.NK.CCL3.CD160 | MT-ND1 | 1.58E−40 | 0.604488713 | 0.871 | 0.728 | 3.64E−36 | 1.66E+35 |
| CD.NK.CCL3.CD160 | JCHAIN | 6.61E−39 | 0.724652813 | 0.783 | 0.534 | 1.52E−34 | 4.76E+33 |
| CD.NK.CCL3.CD160 | IGHA2 | 7.19E−34 | 0.690971747 | 0.435 | 0.157 | 1.66E−29 | 4.17E+28 |
| CD.NK.CCL3.CD160 | HSP90AA1 | 2.58E−33 | 0.718509937 | 0.859 | 0.74 | 5.93E−29 | 1.21E+28 |
| CD.NK.CCL3.CD160 | HSPE1 | 3.87E−27 | 0.678673883 | 0.632 | 0.423 | 8.90E−23 | 7.62E+21 |
| CD.NK.CCL3.CD160 | HSPA1A | 1.93E−26 | 0.7691821 | 0.412 | 0.181 | 4.44E−22 | 1.73E+21 |
| CD.NK.CCL3.CD160 | RPL30 | 6.53E−25 | 0.365461414 | 0.901 | 0.752 | 1.50E−20 | 2.43E+19 |
| CD.NK.CCL3.CD160 | H3F3B | 7.28E−22 | 0.303450824 | 0.967 | 0.929 | 1.68E−17 | 1.81E+16 |
| CD.NK.CCL3.CD160 | RPL9 | 1.74E−21 | 0.4132476 | 0.867 | 0.784 | 4.01E−17 | 1.03E+16 |
| CD.NK.CCL3.CD160 | RGCC | 2.65E−21 | 0.54054654 | 0.588 | 0.372 | 6.11E−17 | 8.85E+15 |
| CD.NK.CCL3.CD160 | HSP90AB1 | 5.94E−21 | 0.540221215 | 0.757 | 0.623 | 1.37E−16 | 3.95E+15 |
| CD.NK.CCL3.CD160 | HOPX | 2.96E−20 | 0.524031331 | 0.472 | 0.25 | 6.81E−16 | 7.70E+14 |
| CD.NK.CCL3.CD160 | AC090498.1 | 7.84E−20 | 0.514850299 | 0.292 | 0.109 | 1.81E−15 | 2.85E+14 |
| CD.NK.CCL3.CD160 | IGLC2 | 2.55E−19 | 0.530241345 | 0.649 | 0.455 | 5.87E−15 | 9.04E+13 |
| CD.NK.CCL3.CD160 | HSPA8 | 7.50E−19 | 0.473556862 | 0.712 | 0.556 | 1.73E−14 | 2.74E+13 |
| CD.NK.CCL3.CD160 | MTRNR2L8 | 5.09E−18 | 0.618162542 | 0.12 | 0.015 | 1.17E−13 | 5.27E+12 |
| CD.NK.CCL3.CD160 | EIF1 | 3.93E−18 | 0.258453968 | 0.945 | 0.896 | 9.05E−14 | 2.86E+12 |
| CD.NK.CCL3.CD160 | DNAJA1 | 1.69E−17 | 0.548386935 | 0.646 | 0.487 | 3.90E−13 | 1.41E+12 |
| CD.NK.CCL3.CD160 | EEF1B2 | 2.03E−17 | 0.443811192 | 0.637 | 0.462 | 4.68E−13 | 9.49E+11 |
| CD.NK.CCL3.CD160 | MT-ND3 | 1.62E−17 | 0.295042902 | 0.907 | 0.803 | 3.73E−13 | 7.92E+11 |
| CD.NK.CCL3.CD160 | DYNLL1 | 3.47E−16 | 0.506419558 | 0.585 | 0.415 | 7.98E−12 | 6.35E+10 |
| CD.NK.CCL3.CD160 | CD160 | 7.74E−16 | 0.484888988 | 0.525 | 0.336 | 1.78E−11 | 2.72E+10 |
| CD.NK.CCL3.CD160 | CACYBP | 9.89E−15 | 0.498886668 | 0.361 | 0.196 | 2.28E−10 | 2.19E+09 |
| CD.NK.CCL3.CD160 | MRPL18 | 1.81E−14 | 0.52610897 | 0.229 | 0.093 | 4.17E−10 | 1.26E+09 |
| CD.NK.CCL3.CD160 | IL32 | 1.87E−14 | 0.377223591 | 0.508 | 0.318 | 4.30E−10 | 8.78E+08 |
| CD.NK.CCL3.CD160 | RPS26 | 1.72E−14 | 0.333672944 | 0.731 | 0.541 | 3.95E−10 | 8.44E+08 |
| CD.NK.CCL3.CD160 | ARHGDIB | 2.46E−14 | 0.359719246 | 0.701 | 0.537 | 5.65E−10 | 6.36E+08 |
| CD.NK.CCL3.CD160 | MIF | 4.59E−14 | 0.444423442 | 0.338 | 0.177 | 1.06E−09 | 4.20E+08 |
| CD.NK.CCL3.CD160 | RPS20 | 1.64E−13 | 0.270264253 | 0.85 | 0.732 | 3.79E−09 | 7.14E+07 |
| CD.NK.CCL3.CD160 | TUBA1A | 4.04E−13 | 0.414276002 | 0.345 | 0.184 | 9.30E−09 | 4.46E+07 |
| CD.NK.CCL3.CD160 | NACA | 5.07E−13 | 0.370412852 | 0.667 | 0.534 | 1.17E−08 | 3.18E+07 |
| CD.NK.CCL3.CD160 | UQCRB | 1.99E−12 | 0.373383606 | 0.504 | 0.328 | 4.58E−08 | 8.15E+06 |
| CD.NK.CCL3.CD160 | HIST1H4C | 2.97E−12 | 0.504306891 | 0.215 | 0.093 | 6.85E−08 | 7.36E+06 |
| CD.NK.CCL3.CD160 | CHCHD2 | 2.27E−12 | 0.357430748 | 0.604 | 0.43 | 5.22E−08 | 6.85E+06 |
| CD.NK.CCL3.CD160 | RPS4Y1 | 5.32E−12 | 0.390402479 | 0.295 | 0.151 | 1.22E−07 | 3.19E+06 |
| CD.NK.CCL3.CD160 | TUBA1B | 7.45E−12 | 0.335657039 | 0.444 | 0.272 | 1.71E−07 | 1.96E+06 |
| CD.NK.CCL3.CD160 | SAP18 | 1.90E−11 | 0.348748978 | 0.449 | 0.284 | 4.36E−07 | 7.99E+05 |
| CD.NK.CCL3.CD160 | RPS17 | 1.51E−11 | 0.257234466 | 0.862 | 0.782 | 3.47E−07 | 7.42E+05 |
| CD.NK.CCL3.CD160 | MRFAP1 | 4.87E−11 | 0.444736714 | 0.238 | 0.117 | 1.12E−06 | 3.97E+05 |
| CD.NK.CCL3.CD160 | SRSF7 | 9.31E−11 | 0.320990444 | 0.637 | 0.486 | 2.14E−06 | 1.50E+05 |
| CD.NK.CCL3.CD160 | ACTG1 | 1.16E−10 | 0.302869206 | 0.707 | 0.568 | 2.66E−06 | 1.14E+05 |
| CD.NK.CCL3.CD160 | RUNX3 | 3.55E−10 | 0.473232721 | 0.254 | 0.135 | 8.17E−06 | 5.79E+04 |
| CD.NK.CCL3.CD160 | ATF4 | 5.53E−10 | 0.418181621 | 0.346 | 0.214 | 1.27E−05 | 3.28E+04 |
| CD.NK.CCL3.CD160 | RNF7 | 5.95E−10 | 0.404897543 | 0.238 | 0.12 | 1.37E−05 | 2.96E+04 |
| CD.NK.CCL3.CD160 | CD48 | 1.19E−09 | 0.35510657 | 0.346 | 0.213 | 2.74E−05 | 1.30E+04 |
| CD.NK.CCL3.CD160 | ZFAS1 | 1.25E−09 | 0.365621322 | 0.332 | 0.202 | 2.88E−05 | 1.27E+04 |
| CD.NK.CCL3.CD160 | SNHG8 | 1.57E−09 | 0.382241035 | 0.257 | 0.14 | 3.62E−05 | 1.06E+04 |
| CD.NK.CCL3.CD160 | EIF1AY | 3.09E−09 | 0.387503983 | 0.13 | 0.048 | 7.11E−05 | 5.45E+03 |
| CD.NK.CCL3.CD160 | FAM177A1 | 3.58E−09 | 0.360075393 | 0.447 | 0.313 | 8.23E−05 | 4.37E+03 |
| CD.NK.CCL3.CD160 | EIF5 | 3.13E−09 | 0.312295908 | 0.454 | 0.312 | 7.20E−05 | 4.33E+03 |
| CD.NK.CCL3.CD160 | RPL22L1 | 3.56E−09 | 0.350483927 | 0.26 | 0.144 | 8.18E−05 | 4.28E+03 |
| CD.NK.CCL3.CD160 | KRT10 | 4.24E−09 | 0.37165121 | 0.277 | 0.161 | 9.76E−05 | 3.81E+03 |
| CD.NK.CCL3.CD160 | G3BP2 | 4.48E−09 | 0.348606528 | 0.323 | 0.195 | 0.000103115 | 3.38E+03 |
| CD.NK.CCL3.CD160 | RPL22 | 5.86E−09 | 0.274719319 | 0.661 | 0.533 | 0.000134777 | 2.04E+03 |
| CD.NK.CCL3.CD160 | CD3E | 8.52E−09 | 0.336921002 | 0.24 | 0.128 | 0.0001961 | 1.72E+03 |
| CD.NK.CCL3.CD160 | ZNF24 | 1.38E−08 | 0.340053133 | 0.119 | 0.044 | 0.000318223 | 1.07E+03 |
| CD.NK.CCL3.CD160 | BCAS2 | 1.40E−08 | 0.292143632 | 0.261 | 0.145 | 0.0003233 | 9.04E+02 |
| CD.NK.CCL3.CD160 | EMC7 | 2.07E−08 | 0.28815856 | 0.145 | 0.06 | 0.000476422 | 6.05E+02 |
| CD.NK.CCL3.CD160 | ABT1 | 2.43E−08 | 0.336962083 | 0.144 | 0.062 | 0.000558447 | 6.03E+02 |
| CD.NK.CCL3.CD160 | CRIP1 | 3.29E−08 | 0.285146989 | 0.249 | 0.14 | 0.000758245 | 3.76E+02 |
| CD.NK.CCL3.CD160 | NCOR1 | 4.92E−08 | 0.363492508 | 0.261 | 0.154 | 0.001131806 | 3.21E+02 |
| CD.NK.CCL3.CD160 | PPIG | 4.55E−08 | 0.278897823 | 0.305 | 0.184 | 0.00104767 | 2.66E+02 |
| CD.NK.CCL3.CD160 | EIF4A3 | 5.52E−08 | 0.333663415 | 0.212 | 0.114 | 0.001270949 | 2.63E+02 |
| CD.NK.CCL3.CD160 | HSPA5 | 6.00E−08 | 0.304848224 | 0.435 | 0.308 | 0.001381657 | 2.21E+02 |
| CD.NK.CCL3.CD160 | DNAJC2 | 1.23E−07 | 0.319906539 | 0.117 | 0.047 | 0.00283314 | 1.13E+02 |
| CD.NK.CCL3.CD160 | GNL3 | 1.14E−07 | 0.294296103 | 0.182 | 0.092 | 0.002627836 | 1.12E+02 |
| CD.NK.CCL3.CD160 | ODC1 | 1.58E−07 | 0.313790319 | 0.111 | 0.044 | 0.003638187 | 8.62E+01 |
| CD.NK.CCL3.CD160 | TSC22D3 | 1.43E−07 | 0.270589245 | 0.611 | 0.491 | 0.003295326 | 8.21E+01 |
| CD.NK.CCL3.CD160 | DNAJC1 | 1.50E−07 | 0.271576028 | 0.175 | 0.086 | 0.003448254 | 7.88E+01 |
| CD.NK.CCL3.CD160 | MANF | 1.96E−07 | 0.345141165 | 0.204 | 0.112 | 0.004521631 | 7.63E+01 |
| CD.NK.CCL3.CD160 | DDX24 | 2.20E−07 | 0.29190189 | 0.364 | 0.242 | 0.005057365 | 5.77E+01 |
| CD.NK.CCL3.CD160 | WHSC1L1 | 2.63E−07 | 0.296396042 | 0.179 | 0.092 | 0.006064484 | 4.89E+01 |
| CD.NK.CCL3.CD160 | DNTTIP2 | 3.09E−07 | 0.324013338 | 0.212 | 0.12 | 0.007106966 | 4.56E+01 |
| CD.NK.CCL3.CD160 | TRAC | 4.37E−07 | 0.368889331 | 0.198 | 0.109 | 0.010064006 | 3.67E+01 |
| CD.NK.CCL3.CD160 | C8orf59 | 3.89E−07 | 0.287067738 | 0.144 | 0.068 | 0.008948787 | 3.21E+01 |
| CD.NK.CCL3.CD160 | VPS37B | 4.78E−07 | 0.257652931 | 0.363 | 0.239 | 0.011003368 | 2.34E+01 |
| CD.NK.CCL3.CD160 | SPINK4 | 5.22E−07 | 0.2784722 | 0.121 | 0.051 | 0.012026951 | 2.32E+01 |
| CD.NK.CCL3.CD160 | GATA3 | 7.07E−07 | 0.309058034 | 0.255 | 0.155 | 0.016267722 | 1.90E+01 |
| CD.NK.CCL3.CD160 | PRDX1 | 1.28E−06 | 0.291811614 | 0.153 | 0.077 | 0.029458562 | 9.91E+00 |
| CD.NK.CCL3.CD160 | GCC2 | 1.30E−06 | 0.270521359 | 0.229 | 0.135 | 0.029854389 | 9.06E+00 |
| CD.NK.CCL3.CD160 | GTF2B | 1.48E−06 | 0.28946028 | 0.161 | 0.083 | 0.034141732 | 8.48E+00 |
| CD.NK.CCL3.CD160 | SNHG12 | 1.42E−06 | 0.257250478 | 0.109 | 0.046 | 0.032631658 | 7.88E+00 |
| CD.NK.CCL3.CD160 | MYADM | 1.97E−06 | 0.335906412 | 0.247 | 0.154 | 0.045431248 | 7.39E+00 |
| CD.NK.CCL3.CD160 | RBM39 | 1.69E−06 | 0.251904859 | 0.564 | 0.457 | 0.038981488 | 6.46E+00 |
| CD.NK.CCL3.CD160 | LSM1 | 2.27E−06 | 0.288194995 | 0.161 | 0.086 | 0.052243431 | 5.52E+00 |
| CD.NK.CCL3.CD160 | UQCRH | 2.85E−06 | 0.25682321 | 0.354 | 0.245 | 0.065633227 | 3.91E+00 |
| CD.NK.CCL3.CD160 | IMP3 | 3.18E−06 | 0.273356228 | 0.186 | 0.104 | 0.073212195 | 3.73E+00 |
| CD.NK.CCL3.CD160 | CIR1 | 5.93E−06 | 0.265842387 | 0.184 | 0.105 | 0.136493847 | 1.95E+00 |
| CD.NK.CCL3.CD160 | PBDC1 | 9.87E−06 | 0.255690673 | 0.104 | 0.047 | 0.227256923 | 1.13E+00 |
| CD.NK.CCL3.CD160 | HSPD1 | 2.21E−05 | 0.410710748 | 0.406 | 0.335 | 0.507776098 | 8.09E−01 |
| CD.NK.CCL3.CD160 | SNRPA1 | 1.89E−05 | 0.26247907 | 0.12 | 0.06 | 0.435380399 | 6.03E−01 |
| CD.NK.CCL3.CD160 | MPC2 | 1.98E−05 | 0.265589821 | 0.163 | 0.093 | 0.454693404 | 5.84E−01 |
| CD.NK.CCL3.CD160 | ZFAND2A | 2.76E−05 | 0.252346461 | 0.101 | 0.047 | 0.635858897 | 3.97E−01 |
| CD.NK.CCL3.CD160 | PDCL3 | 3.13E−05 | 0.270723644 | 0.165 | 0.098 | 0.721215448 | 3.75E−01 |
| CD.NK.CCL3.CD160 | LCK | 3.35E−05 | 0.273346555 | 0.203 | 0.127 | 0.770946308 | 3.55E−01 |
| CD.NK.CCL3.CD160 | CREM | 0.000375254 | 0.345713432 | 0.579 | 0.539 | 1 | 3.46E−01 |
| CD.NK.CCL3.CD160 | DNAJB6 | 3.26E−05 | 0.256164349 | 0.505 | 0.401 | 0.751413879 | 3.41E−01 |
| CD.NK.CCL3.CD160 | CCDC59 | 7.95E−05 | 0.279753774 | 0.207 | 0.135 | 1 | 2.80E−01 |
| CD.NK.CCL3.CD160 | TSPYL2 | 6.82E−05 | 0.266065806 | 0.179 | 0.109 | 1 | 2.66E−01 |
| CD.NK.CCL3.CD160 | HSPH1 | 9.88E−05 | 0.263714442 | 0.286 | 0.204 | 1 | 2.64E−01 |
| CD.NK.CCL3.CD160 | ATP1B3 | 0.000239274 | 0.251398073 | 0.413 | 0.329 | 1 | 2.51E−01 |
| CD.NK.CCL3.CD160 | ZC3H12A | 0.049236526 | −0.250685921 | 0.086 | 0.11 | 1 | −2.51E−01 |
| CD.NK.CCL3.CD160 | MAPK1 | 0.000292318 | −0.250792725 | 0.18 | 0.248 | 1 | −2.51E−01 |
| CD.NK.CCL3.CD160 | PRKACB | 0.012459444 | −0.251083965 | 0.113 | 0.149 | 1 | −2.51E−01 |
| CD.NK.CCL3.CD160 | ERGIC2 | 0.005672173 | −0.251228814 | 0.101 | 0.14 | 1 | −2.51E−01 |
| CD.NK.CCL3.CD160 | PRKAR1A | 0.005273828 | −0.251367859 | 0.171 | 0.215 | 1 | −2.51E−01 |
| CD.NK.CCL3.CD160 | SLC38A1 | 0.007492737 | −0.251441165 | 0.226 | 0.265 | 1 | −2.51E−01 |
| CD.NK.CCL3.CD160 | LBR | 0.001252473 | −0.251608096 | 0.087 | 0.132 | 1 | −2.52E−01 |
| CD.NK.CCL3.CD160 | CLDND1 | 0.001784837 | −0.252502153 | 0.385 | 0.44 | 1 | −2.53E−01 |
| CD.NK.CCL3.CD160 | CD47 | 0.007260408 | −0.253206388 | 0.198 | 0.238 | 1 | −2.53E−01 |
| CD.NK.CCL3.CD160 | HNRNPH2 | 0.004374348 | −0.253867361 | 0.102 | 0.141 | 1 | −2.54E−01 |
| CD.NK.CCL3.CD160 | KDELR2 | 0.023186618 | −0.254258628 | 0.192 | 0.221 | 1 | −2.54E−01 |
| CD.NK.CCL3.CD160 | PRKCH | 0.005589356 | −0.254627633 | 0.172 | 0.219 | 1 | −2.55E−01 |
| CD.NK.CCL3.CD160 | FAM49B | 0.000964255 | −0.254976616 | 0.381 | 0.429 | 1 | −2.55E−01 |
| CD.NK.CCL3.CD160 | SLC16A3 | 0.026900442 | −0.256730023 | 0.137 | 0.168 | 1 | −2.57E−01 |
| CD.NK.CCL3.CD160 | JMJD1C | 0.004898188 | −0.257042758 | 0.117 | 0.161 | 1 | −2.57E−01 |
| CD.NK.CCL3.CD160 | CCND2 | 0.006824206 | −0.25725804 | 0.215 | 0.258 | 1 | −2.57E−01 |
| CD.NK.CCL3.CD160 | ZBTB38 | 0.002650181 | −0.257571011 | 0.103 | 0.146 | 1 | −2.58E−01 |
| CD.NK.CCL3.CD160 | SAMD3 | 0.007997113 | −0.257604793 | 0.159 | 0.201 | 1 | −2.58E−01 |
| CD.NK.CCL3.CD160 | KIAA1551 | 0.006449389 | −0.257765261 | 0.131 | 0.173 | 1 | −2.58E−01 |
| CD.NK.CCL3.CD160 | BST2 | 0.000337231 | −0.258243335 | 0.286 | 0.345 | 1 | −2.58E−01 |
| CD.NK.CCL3.CD160 | MAT2B | 0.014581197 | −0.258466013 | 0.103 | 0.138 | 1 | −2.58E−01 |
| CD.NK.CCL3.CD160 | TCIRG1 | 0.020156325 | −0.258882017 | 0.086 | 0.116 | 1 | −2.59E−01 |
| CD.NK.CCL3.CD160 | CELF2 | 0.001203644 | −0.259659718 | 0.237 | 0.29 | 1 | −2.60E−01 |
| CD.NK.CCL3.CD160 | OSBPL8 | 0.025420072 | −0.25976736 | 0.113 | 0.145 | 1 | −2.60E−01 |
| CD.NK.CCL3.CD160 | DNAJB9 | 0.00040637 | −0.25983854 | 0.168 | 0.229 | 1 | −2.60E−01 |
| CD.NK.CCL3.CD160 | MT2A | 0.015399377 | −0.259851539 | 0.152 | 0.189 | 1 | −2.60E−01 |
| CD.NK.CCL3.CD160 | PYURF | 0.000200796 | −0.261873212 | 0.192 | 0.256 | 1 | −2.62E−01 |
| CD.NK.CCL3.CD160 | NDUFA11 | 0.001286317 | −0.26245973 | 0.181 | 0.236 | 1 | −2.62E−01 |
| CD.NK.CCL3.CD160 | TMED2 | 0.002242407 | −0.262494177 | 0.183 | 0.233 | 1 | −2.62E−01 |
| CD.NK.CCL3.CD160 | ERBB2IP | 0.008411088 | −0.26263996 | 0.112 | 0.15 | 1 | −2.63E−01 |
| CD.NK.CCL3.CD160 | SETX | 0.040961222 | −0.26299053 | 0.113 | 0.14 | 1 | −2.63E−01 |
| CD.NK.CCL3.CD160 | IVNS1ABP | 0.003637244 | −0.263141773 | 0.162 | 0.209 | 1 | −2.63E−01 |
| CD.NK.CCL3.CD160 | SRSF6 | 0.009013723 | −0.263805225 | 0.138 | 0.175 | 1 | −2.64E−01 |
| CD.NK.CCL3.CD160 | CD96 | 0.00100044 | −0.265488851 | 0.276 | 0.329 | 1 | −2.65E−01 |
| CD.NK.CCL3.CD160 | RAB7A | 0.007114375 | −0.26877905 | 0.177 | 0.218 | 1 | −2.69E−01 |
| CD.NK.CCL3.CD160 | PLEK | 0.000316647 | −0.269103596 | 0.141 | 0.203 | 1 | −2.69E−01 |
| CD.NK.CCL3.CD160 | BHLHE40 | 0.000858588 | −0.269565923 | 0.115 | 0.167 | 1 | −2.70E−01 |
| CD.NK.CCL3.CD160 | LAMTOR4 | 0.00257577 | −0.271602343 | 0.235 | 0.279 | 1 | −2.72E−01 |
| CD.NK.CCL3.CD160 | ATP5J2 | 0.000556921 | −0.271816104 | 0.219 | 0.281 | 1 | −2.72E−01 |
| CD.NK.CCL3.CD160 | EEF2 | 0.000353605 | −0.272085879 | 0.347 | 0.395 | 1 | −2.72E−01 |
| CD.NK.CCL3.CD160 | MGEA5 | 0.013390277 | −0.273688126 | 0.15 | 0.187 | 1 | −2.74E−01 |
| CD.NK.CCL3.CD160 | EIF5A | 0.000789841 | −0.273880334 | 0.152 | 0.21 | 1 | −2.74E−01 |
| CD.NK.CCL3.CD160 | SAR1A | 0.019016566 | −0.273895966 | 0.18 | 0.214 | 1 | −2.74E−01 |
| CD.NK.CCL3.CD160 | TGFB1 | 0.03886488 | −0.275288783 | 0.125 | 0.152 | 1 | −2.75E−01 |
| CD.NK.CCL3.CD160 | PTPN12 | 0.014336269 | −0.275409655 | 0.081 | 0.112 | 1 | −2.75E−01 |
| CD.NK.CCL3.CD160 | HNRNPU | 0.000135036 | −0.275670337 | 0.27 | 0.34 | 1 | −2.76E−01 |
| CD.NK.CCL3.CD160 | NDUFB2 | 0.00118594 | −0.276437623 | 0.295 | 0.347 | 1 | −2.76E−01 |
| CD.NK.CCL3.CD160 | GOLGA7 | 0.000814433 | −0.280340194 | 0.086 | 0.133 | 1 | −2.80E−01 |
| CD.NK.CCL3.CD160 | B3GNT7 | 0.019783121 | −0.280948388 | 0.273 | 0.312 | 1 | −2.81E−01 |
| CD.NK.CCL3.CD160 | DDX39B | 0.009548516 | −0.280961267 | 0.081 | 0.115 | 1 | −2.81E−01 |
| CD.NK.CCL3.CD160 | RASGEF1B | 0.028115836 | −0.28170366 | 0.196 | 0.226 | 1 | −2.82E−01 |
| CD.NK.CCL3.CD160 | ADAM10 | 0.001353464 | −0.285057515 | 0.071 | 0.112 | 1 | −2.85E−01 |
| CD.NK.CCL3.CD160 | SMG1 | 0.0036654 | −0.28612673 | 0.087 | 0.127 | 1 | −2.86E−01 |
| CD.NK.CCL3.CD160 | GDI2 | 0.004474238 | −0.286223984 | 0.126 | 0.168 | 1 | −2.86E−01 |
| CD.NK.CCL3.CD160 | HMGN3 | 9.10E−05 | −0.289457041 | 0.268 | 0.331 | 1 | −2.89E−01 |
| CD.NK.CCL3.CD160 | KRT81 | 0.001608232 | −0.292977279 | 0.108 | 0.154 | 1 | −2.93E−01 |
| CD.NK.CCL3.CD160 | RP11-51J9.5 | 0.001013445 | −0.293089144 | 0.077 | 0.121 | 1 | −2.93E−01 |
| CD.NK.CCL3.CD160 | ECH1 | 0.000435529 | −0.29327237 | 0.169 | 0.229 | 1 | −2.93E−01 |
| CD.NK.CCL3.CD160 | COPE | 5.26E−05 | −0.293532934 | 0.234 | 0.306 | 1 | −2.94E−01 |
| CD.NK.CCL3.CD160 | TOP2B | 0.000271515 | −0.294531506 | 0.099 | 0.154 | 1 | −2.95E−01 |
| CD.NK.CCL3.CD160 | C7orf73 | 0.000278264 | −0.294814853 | 0.112 | 0.169 | 1 | −2.95E−01 |
| CD.NK.CCL3.CD160 | GLIPR1 | 0.001582692 | −0.295514711 | 0.21 | 0.261 | 1 | −2.96E−01 |
| CD.NK.CCL3.CD160 | SACM1L | 0.003905704 | −0.295575987 | 0.134 | 0.178 | 1 | −2.96E−01 |
| CD.NK.CCL3.CD160 | MED30 | 0.004865866 | −0.296662278 | 0.115 | 0.157 | 1 | −2.97E−01 |
| CD.NK.CCL3.CD160 | TFDP2 | 0.022576552 | −0.297841604 | 0.104 | 0.133 | 1 | −2.98E−01 |
| CD.NK.CCL3.CD160 | NFE2L2 | 0.003690743 | −0.299451753 | 0.262 | 0.305 | 1 | −2.99E−01 |
| CD.NK.CCL3.CD160 | PSMA3-AS1 | 0.000285592 | −0.299674778 | 0.166 | 0.226 | 1 | −3.00E−01 |
| CD.NK.CCL3.CD160 | ISG20 | 5.12E−05 | −0.299923566 | 0.237 | 0.311 | 1 | −3.00E−01 |
| CD.NK.CCL3.CD160 | CD164 | 0.003311639 | −0.300293358 | 0.274 | 0.313 | 1 | −3.00E−01 |
| CD.NK.CCL3.CD160 | S100A11 | 0.020524077 | −0.30199254 | 0.261 | 0.29 | 1 | −3.02E−01 |
| CD.NK.CCL3.CD160 | TRAF3IP3 | 0.000104981 | −0.303443931 | 0.182 | 0.252 | 1 | −3.03E−01 |
| CD.NK.CCL3.CD160 | MBNL1 | 0.014561667 | −0.305891688 | 0.257 | 0.285 | 1 | −3.06E−01 |
| CD.NK.CCL3.CD160 | TMBIM4 | 0.000273167 | −0.309143667 | 0.13 | 0.187 | 1 | −3.09E−01 |
| CD.NK.CCL3.CD160 | TPM4 | 0.006470979 | −0.309343993 | 0.17 | 0.21 | 1 | −3.09E−01 |
| CD.NK.CCL3.CD160 | SLFN13 | 0.00044546 | −0.310999352 | 0.074 | 0.12 | 1 | −3.11E−01 |
| CD.NK.CCL3.CD160 | EPS15 | 9.83E−05 | −0.314267201 | 0.06 | 0.11 | 1 | −3.14E−01 |
| CD.NK.CCL3.CD160 | ITGB2 | 9.68E−05 | −0.314553742 | 0.202 | 0.27 | 1 | −3.15E−01 |
| CD.NK.CCL3.CD160 | FOXP1 | 0.000303124 | −0.318939521 | 0.088 | 0.139 | 1 | −3.19E−01 |
| CD.NK.CCL3.CD160 | HNRNPH1 | 0.000620568 | −0.320379526 | 0.251 | 0.311 | 1 | −3.20E−01 |
| CD.NK.CCL3.CD160 | LYST | 0.000136074 | −0.32118844 | 0.151 | 0.215 | 1 | −3.21E−01 |
| CD.NK.CCL3.CD160 | VAMP5 | 0.000286855 | −0.321783453 | 0.12 | 0.175 | 1 | −3.22E−01 |
| CD.NK.CCL3.CD160 | TANK | 0.001172138 | −0.323244773 | 0.102 | 0.148 | 1 | −3.23E−01 |
| CD.NK.CCL3.CD160 | EPAS1 | 0.000224089 | −0.336161506 | 0.075 | 0.125 | 1 | −3.36E−01 |
| CD.NK.CCL3.CD160 | SNX14 | 6.46E−05 | −0.336439258 | 0.054 | 0.103 | 1 | −3.36E−01 |
| CD.NK.CCL3.CD160 | TSTD1 | 0.000226935 | −0.338458474 | 0.156 | 0.215 | 1 | −3.38E−01 |
| CD.NK.CCL3.CD160 | RP11-347P5.1 | 0.000104564 | −0.339417549 | 0.114 | 0.174 | 1 | −3.39E−01 |
| CD.NK.CCL3.CD160 | TOX | 8.91E−05 | −0.34292246 | 0.098 | 0.155 | 1 | −3.43E−01 |
| CD.NK.CCL3.CD160 | ZC3HAV1 | 0.000226185 | −0.344806661 | 0.14 | 0.197 | 1 | −3.45E−01 |
| CD.NK.CCL3.CD160 | PYHIN1 | 5.05E−05 | −0.345959915 | 0.301 | 0.369 | 1 | −3.46E−01 |
| CD.NK.CCL3.CD160 | RORA | 4.25E−05 | −0.352311322 | 0.274 | 0.345 | 0.979260129 | −3.60E−01 |
| CD.NK.CCL3.CD160 | MGAT4A | 0.00073221 | −0.360228862 | 0.094 | 0.14 | 1 | −3.60E−01 |
| CD.NK.CCL3.CD160 | FOSL2 | 3.73E−05 | −0.316821042 | 0.242 | 0.316 | 0.85790303 | −3.69E−01 |
| CD.NK.CCL3.CD160 | IGHG3 | 0.000176491 | −0.406917901 | 0.068 | 0.117 | 1 | −4.07E−01 |
| CD.NK.CCL3.CD160 | CTNNB1 | 0.000114499 | −0.416005759 | 0.088 | 0.143 | 1 | −4.16E−01 |
| CD.NK.CCL3.CD160 | HSPA1B | 0.003234509 | −0.423169423 | 0.109 | 0.151 | 1 | −4.23E−01 |
| CD.NK.CCL3.CD160 | COX6B1 | 2.87E−05 | −0.306282925 | 0.299 | 0.37 | 0.659563587 | −4.64E−01 |
| CD.NK.CCL3.CD160 | ARPC5 | 2.76E−05 | −0.300944423 | 0.251 | 0.328 | 0.636117348 | −4.73E−01 |
| CD.NK.CCL3.CD160 | CCL3L3 | 0.157959241 | −0.473542948 | 0.089 | 0.106 | 1 | −4.74E−01 |
| CD.NK.CCL3.CD160 | CST3 | 3.23E−05 | −0.390205884 | 0.075 | 0.133 | 0.742517773 | −5.26E−01 |
| CD.NK.CCL3.CD160 | EGR1 | 0.022742819 | −0.537410576 | 0.087 | 0.115 | 1 | −5.37E−01 |
| CD.NK.CCL3.CD160 | NFKBIZ | 2.96E−05 | −0.47106985 | 0.142 | 0.208 | 0.681716662 | −6.91E−01 |
| CD.NK.CCL3.CD160 | OGT | 1.84E−05 | −0.324736336 | 0.059 | 0.115 | 0.423697762 | −7.66E−01 |
| CD.NK.CCL3.CD160 | ANXA2 | 1.81E−05 | −0.333316028 | 0.111 | 0.179 | 0.415993541 | −8.01E−01 |
| CD.NK.CCL3.CD160 | SLC38A2 | 1.92E−05 | −0.357063654 | 0.11 | 0.177 | 0.441625718 | −8.09E−01 |
| CD.NK.CCL3.CD160 | STT3B | 1.59E−05 | −0.301747252 | 0.093 | 0.16 | 0.366061896 | −8.24E−01 |
| CD.NK.CCL3.CD160 | LNPEP | 1.70E−05 | −0.327080519 | 0.111 | 0.18 | 0.390477793 | −8.38E−01 |
| CD.NK.CCL3.CD160 | C1QA | 1.50E−05 | −0.347986721 | 0.053 | 0.108 | 0.344183514 | −1.01E+00 |
| CD.NK.CCL3.CD160 | ACTR3 | 1.36E−05 | −0.329808965 | 0.296 | 0.368 | 0.313371959 | −1.05E+00 |
| CD.NK.CCL3.CD160 | SKIL | 1.00E−05 | −0.358650756 | 0.201 | 0.278 | 0.231278279 | −1.55E+00 |
| CD.NK.CCL3.CD160 | ETS1 | 8.39E−06 | −0.320133682 | 0.205 | 0.29 | 0.19305443 | −1.66E+00 |
| CD.NK.CCL3.CD160 | C6orf62 | 7.74E−06 | −0.345276841 | 0.119 | 0.191 | 0.178180693 | −1.94E+00 |
| CD.NK.CCL3.CD160 | CH17-189H20.1 | 8.87E−06 | −0.403244619 | 0.141 | 0.213 | 0.204235767 | −1.97E+00 |
| CD.NK.CCL3.CD160 | C1QB | 6.02E−06 | −0.406422729 | 0.05 | 0.105 | 0.138587316 | −2.93E+00 |
| CD.NK.CCL3.CD160 | MPG | 5.01E−06 | −0.375406121 | 0.116 | 0.187 | 0.115349778 | −3.25E+00 |
| CD.NK.CCL3.CD160 | GMFG | 3.12E−06 | −0.305345908 | 0.36 | 0.434 | 0.071722725 | −4.26E+00 |
| CD.NK.CCL3.CD160 | LCP1 | 2.39E−06 | −0.252437622 | 0.385 | 0.48 | 0.054950412 | −4.59E+00 |
| CD.NK.CCL3.CD160 | ADAM28 | 3.22E−06 | −0.351159417 | 0.062 | 0.125 | 0.074094071 | −4.74E+00 |
| CD.NK.CCL3.CD160 | ATP5G2 | 2.57E−06 | −0.305687267 | 0.417 | 0.485 | 0.059130989 | −5.17E+00 |
| CD.NK.CCL3.CD160 | PDCD4 | 2.49E−06 | −0.332293909 | 0.354 | 0.439 | 0.057356812 | −5.79E+00 |
| CD.NK.CCL3.CD160 | PPP1R15A | 1.59E−06 | −0.432624676 | 0.268 | 0.358 | 0.036692108 | −1.18E+01 |
| CD.NK.CCL3.CD160 | YPEL3 | 1.38E−06 | −0.404045957 | 0.054 | 0.116 | 0.031872586 | −1.27E+01 |
| CD.NK.CCL3.CD160 | GPR65 | 9.71E−07 | −0.347554585 | 0.412 | 0.492 | 0.022345071 | −1.56E+01 |
| CD.NK.CCL3.CD160 | GNLY | 1.46E−06 | −0.534243944 | 0.186 | 0.274 | 0.033520912 | −1.59E+01 |
| CD.NK.CCL3.CD160 | CDC42SE2 | 8.34E−07 | −0.42008483 | 0.221 | 0.305 | 0.019204265 | −2.19E+01 |
| CD.NK.CCL3.CD160 | INTS6 | 6.61E−07 | −0.382037264 | 0.11 | 0.19 | 0.015208284 | −2.51E+01 |
| CD.NK.CCL3.CD160 | CLIC3 | 5.46E−07 | −0.384757408 | 0.368 | 0.446 | 0.012557682 | −3.06E+01 |
| CD.NK.CCL3.CD160 | IFNGR1 | 5.21E−07 | −0.367890757 | 0.172 | 0.262 | 0.011995073 | −3.07E+01 |
| CD.NK.CCL3.CD160 | GZMB | 2.28E−07 | −0.417576555 | 0.377 | 0.468 | 0.0052426 | −7.97E+01 |
| CD.NK.CCL3.CD160 | RPL36A | 1.07E−07 | −0.359132364 | 0.276 | 0.38 | 0.002451541 | −1.46E+02 |
| CD.NK.CCL3.CD160 | CHIC2 | 9.90E−08 | −0.459944531 | 0.09 | 0.169 | 0.002279496 | −2.02E+02 |
| CD.NK.CCL3.CD160 | SPON2 | 9.16E−08 | −0.451550572 | 0.104 | 0.189 | 0.002109158 | −2.14E+02 |
| CD.NK.CCL3.CD160 | HCST | 3.01E−08 | −0.256360208 | 0.642 | 0.711 | 0.000692943 | −3.70E+02 |
| CD.NK.CCL3.CD160 | ALOX5AP | 2.18E−08 | −0.34602636 | 0.54 | 0.601 | 0.000502445 | −6.89E+02 |
| CD.NK.CCL3.CD160 | CD74 | 1.34E−08 | −0.401904209 | 0.465 | 0.55 | 0.000308101 | −1.30E+03 |
| CD.NK.CCL3.CD160 | MT-ND4L | 1.11E−08 | −0.446692941 | 0.283 | 0.385 | 0.000256039 | −1.74E+03 |
| CD.NK.CCL3.CD160 | PIP4K2A | 7.73E−09 | −0.357885561 | 0.203 | 0.314 | 0.000177994 | −2.01E+03 |
| CD.NK.CCL3.CD160 | CEBPD | 9.70E−09 | −0.505499073 | 0.132 | 0.231 | 0.000223202 | −2.26E+03 |
| CD.NK.CCL3.CD160 | ITM2B | 2.23E−09 | −0.282765609 | 0.662 | 0.716 | 5.13E−05 | −5.51E+03 |
| CD.NK.CCL3.CD160 | SDCBP | 3.31E−09 | −0.44437979 | 0.34 | 0.44 | 7.62E−05 | −5.83E+03 |
| CD.NK.CCL3.CD160 | PTPRC | 7.98E−10 | −0.300753018 | 0.666 | 0.748 | 1.84E−05 | −1.64E+04 |
| CD.NK.CCL3.CD160 | MT-ND5 | 2.39E−10 | −0.352010555 | 0.656 | 0.723 | 5.50E−06 | −6.40E+04 |
| CD.NK.CCL3.CD160 | NEAT1 | 1.99E−10 | −0.436290686 | 0.48 | 0.572 | 4.58E−06 | −9.53E+04 |
| CD.NK.CCL3.CD160 | TYROBP | 4.40E−11 | −0.274203931 | 0.767 | 0.828 | 1.01E−06 | −2.71E+05 |
| CD.NK.CCL3.CD160 | HLA-DRB1 | 5.71E−11 | −0.501129585 | 0.11 | 0.221 | 1.31E−06 | −3.81E+05 |
| CD.NK.CCL3.CD160 | KLRB1 | 8.68E−12 | −0.258385666 | 0.866 | 0.901 | 2.00E−07 | −1.29E+06 |
| CD.NK.CCL3.CD160 | EVL | 5.14E−12 | −0.355105335 | 0.53 | 0.638 | 1.18E−07 | −3.00E+06 |
| CD.NK.CCL3.CD160 | SRGN | 2.85E−12 | −0.289025506 | 0.935 | 0.938 | 6.57E−08 | −4.40E+06 |
| CD.NK.CCL3.CD160 | ARF6 | 7.94E−13 | −0.504444125 | 0.303 | 0.435 | 1.83E−08 | −2.76E+07 |
| CD.NK.CCL3.CD160 | SH2D1A | 1.58E−13 | −0.50560203 | 0.284 | 0.426 | 3.65E−09 | −1.39E+08 |
| CD.NK.CCL3.CD160 | GZMH | 1.85E−13 | −0.664673354 | 0.084 | 0.202 | 4.26E−09 | −1.56E+08 |
| CD.NK.CCL3.CD160 | CCL3 | 1.03E−13 | −0.575130121 | 0.416 | 0.591 | 2.37E−09 | −2.43E+08 |
| CD.NK.CCL3.CD160 | KLRF1 | 1.75E−14 | −0.48000976 | 0.472 | 0.593 | 4.03E−10 | −1.19E+09 |
| CD.NK.CCL3.CD160 | PFN1 | 7.52E−15 | −0.257625736 | 0.903 | 0.915 | 1.73E−10 | −1.49E+09 |
| CD.NK.CCL3.CD160 | SERF2 | 1.11E−15 | −0.306238963 | 0.713 | 0.781 | 2.56E−11 | −1.20E+10 |
| CD.NK.CCL3.CD160 | RPS2 | 2.99E−17 | −0.258099414 | 0.908 | 0.93 | 6.89E−13 | −3.75E+11 |
| CD.NK.CCL3.CD160 | KLRC1 | 5.52E−21 | −0.606558945 | 0.267 | 0.472 | 1.27E−16 | −4.78E+15 |
| CD.NK.CCL3.CD160 | KLRD1 | 5.16E−23 | −0.480234163 | 0.684 | 0.793 | 1.19E−18 | −4.04E+17 |
| CD.NK.CCL3.CD160 | AREG | 1.62E−23 | −0.638267424 | 0.559 | 0.701 | 3.72E−19 | −1.71E+18 |
| CD.NK.CCL3.CD160 | TMSB10 | 3.96E−24 | −0.30427132 | 0.91 | 0.953 | 9.12E−20 | −3.33E+18 |
| CD.NK.CCL3.CD160 | NKG7 | 2.41E−26 | −0.32745046 | 0.919 | 0.946 | 5.55E−22 | −5.90E+20 |
| CD.NK.CCL3.CD160 | CMC1 | 2.34E−30 | −0.677153946 | 0.582 | 0.74 | 5.39E−26 | −1.26E+25 |
| CD.NK.CCL3.CD160 | XIST | 1.10E−32 | −0.980002196 | 0.063 | 0.262 | 2.53E−28 | −3.88E+27 |

TABLE 3
CD_EndClusterCPM_PCA_Loadings_Combo.Tells.Mloid.Epith.

| | PC1 | PC2 | PC3 | PC4 | PC5 | PC6 | PC7 |
|---|---|---|---|---|---|---|---|
| variance | 0.14300 | 0.14143 | 0.10339 | 0.09765 | 0.08085 | 0.07428 | 0.06800 |
| T.CCR7.GPR183 | 0.12995 | 0.05826 | 0.00097 | 0.12833 | −0.01378 | 0.11251 | −0.00909 |
| T.MKI67.RRM2 | 0.12322 | 0.00451 | 0.00353 | 0.03893 | −0.08176 | −0.05509 | −0.06957 |
| cDC2.CLEC10A.CD1E | 0.11344 | 0.06972 | −0.02822 | 0.03897 | −0.00473 | 0.06927 | −0.14819 |
| cDC2.CD1C.NDRG2 | 0.10780 | 0.07303 | −0.04446 | −0.03176 | −0.03833 | −0.09222 | −0.15874 |
| T.GNLY.GZMH | 0.10223 | −0.01927 | −0.07081 | −0.00289 | −0.13751 | −0.09358 | 0.10941 |
| T.MKI67.MAF | 0.09261 | 0.04324 | −0.03939 | 0.12187 | −0.05791 | 0.08555 | 0.08550 |
| T.CCL20.IFNG | 0.09137 | −0.05943 | −0.07186 | −0.06404 | −0.06861 | −0.11184 | −0.06240 |
| T.CCL4.GMZK | 0.09017 | 0.04742 | 0.01718 | 0.12933 | −0.07864 | −0.01157 | 0.15968 |
| T.IFI44L.PTGER4 | 0.08871 | 0.07376 | 0.01168 | 0.11517 | −0.05430 | −0.00482 | 0.09970 |
| T.GNLY.CTSW | 0.08829 | 0.02911 | −0.04324 | −0.03667 | −0.08025 | −0.14810 | −0.07266 |
| T.CD8B.TRGC2 | 0.08674 | 0.02330 | −0.07123 | −0.07771 | −0.07011 | −0.15095 | −0.03070 |
| T.GNLY.IFNG | 0.08187 | 0.02010 | −0.03985 | −0.03537 | −0.08380 | −0.17737 | −0.04953 |
| Mcell.CCL23.SPIB | 0.08042 | 0.06303 | 0.02450 | 0.15312 | −0.05507 | 0.03799 | 0.13352 |
| T.KLRB1.LTF | 0.07883 | 0.03245 | −0.05906 | −0.07359 | −0.07486 | −0.17163 | −0.06590 |
| T.LAG3.BATF | 0.07819 | 0.06800 | −0.00950 | 0.00987 | 0.00321 | −0.00871 | −0.09281 |
| Mono.cDC2.CLEC10A.AREG | 0.07805 | 0.04005 | −0.04674 | −0.05056 | −0.06841 | −0.16960 | −0.09025 |
| T.MKI67.ITGB7 | 0.07704 | 0.04173 | 0.01624 | 0.14657 | −0.06044 | 0.01799 | 0.16596 |
| Mac.DC.CXCL8.NLRP3 | 0.07371 | 0.01242 | −0.06570 | −0.07291 | −0.06776 | −0.17608 | −0.04316 |
| EC.IFI6.IFI27 | 0.07182 | 0.04152 | 0.01257 | 0.14930 | −0.05403 | 0.03503 | 0.16612 |
| ILC.KRT81.IL22 | 0.07069 | 0.03808 | 0.01873 | 0.14387 | −0.05603 | 0.02103 | 0.18096 |
| T.CCL20.IL22 | 0.07043 | 0.03721 | 0.01698 | 0.14610 | −0.05642 | 0.01394 | 0.18022 |
| T.IFI6.IRF7 | 0.06979 | 0.04146 | 0.01923 | 0.14657 | −0.05801 | 0.01395 | 0.17710 |
| Secretory.REG3G.IFI6 | 0.06942 | 0.03812 | 0.01868 | 0.14702 | −0.05620 | 0.01512 | 0.17931 |
| EC.TCL1A.IFI6 | 0.06684 | 0.03621 | 0.02018 | 0.14990 | −0.05794 | 0.01509 | 0.17770 |
| T.TRDC.TRGC2 | 0.06540 | −0.04416 | −0.06727 | −0.08265 | −0.06622 | −0.10443 | −0.07824 |
| T.CCR7.SELL | 0.06412 | 0.04423 | −0.02886 | −0.00211 | 0.03735 | 0.10500 | −0.14512 |
| Mac.FOLR2.LILRB5 | 0.06398 | 0.03847 | −0.03135 | −0.05770 | −0.07335 | −0.17841 | −0.05961 |
| EC.SLC28A2.GSTA2 | 0.05954 | 0.06295 | −0.00418 | 0.01845 | 0.02201 | 0.01470 | −0.09080 |
| ILC.LST1.AREG | 0.05897 | 0.04779 | −0.00517 | −0.11574 | −0.08282 | −0.11719 | −0.04994 |
| T.MKI67.CCR7 | 0.05767 | 0.05950 | 0.00142 | 0.01265 | 0.01740 | 0.04634 | −0.08386 |
| ILC.IL22.KIT | 0.05576 | 0.05495 | −0.00707 | 0.01232 | 0.02176 | 0.01145 | −0.08768 |
| T.CARD16.GB2 | 0.04658 | −0.11926 | −0.01706 | −0.09021 | −0.01715 | 0.02666 | 0.02704 |
| T.MKI67.NKG7 | 0.04554 | 0.04082 | −0.01737 | 0.15047 | −0.08132 | −0.01040 | 0.17634 |
| T.EGR1.TNF | 0.04526 | 0.04012 | −0.07511 | −0.02409 | −0.10262 | −0.18762 | −0.02351 |
| T.EGR1.IFNG | 0.04352 | 0.06396 | −0.03120 | 0.00844 | −0.04593 | 0.06313 | −0.10846 |
| Mono.Mac.CXCL10.FCN1 | 0.04212 | −0.13905 | −0.07980 | −0.06924 | 0.02184 | 0.02385 | 0.05472 |
| Mac.APOE.PTGDS | 0.03908 | −0.09822 | 0.07280 | −0.09185 | −0.08799 | 0.06183 | 0.00322 |
| NK.GNLY.KLRC1 | 0.03852 | −0.10560 | −0.09482 | −0.08310 | 0.04361 | 0.06689 | 0.05270 |
| Mcell.CSRP2.SPIB | 0.03745 | 0.08053 | 0.03035 | −0.04935 | −0.00949 | 0.01845 | −0.04759 |
| Mac.SEPP1.CXCL3 | 0.03684 | −0.01376 | −0.00352 | −0.08837 | −0.07751 | −0.17322 | 0.03530 |
| Mac.C1QB.FCGR1A | 0.03631 | −0.03412 | −0.05763 | −0.01396 | 0.04297 | 0.10946 | −0.05300 |
| Mono.Mac.CXCL10.LYZ | 0.03514 | −0.08443 | −0.08914 | −0.05355 | 0.02794 | 0.00192 | 0.04808 |
| Goblet.S100P.FCGBP | 0.03266 | −0.00186 | 0.00138 | 0.00726 | 0.01526 | 0.18207 | −0.10501 |
| Mac.CXCL2.TNF | 0.03256 | −0.06473 | −0.08739 | −0.05578 | 0.01233 | −0.01764 | 0.05849 |
| EC.LECT1.FAM26F | 0.03221 | 0.06141 | 0.00918 | 0.00345 | 0.00589 | 0.00381 | −0.10297 |
| EpithStem.LINC00176.RPS4Y1 | 0.03167 | 0.06440 | 0.01219 | 0.00529 | 0.01040 | 0.03014 | −0.10673 |
| Mono.Mac.CXCL10.CXCL11 | 0.03166 | −0.12291 | −0.10957 | −0.08115 | 0.03928 | 0.03372 | 0.07975 |
| EC.AKR1C1.TSPAN1 | 0.03141 | 0.01000 | −0.00113 | 0.01736 | 0.01424 | 0.16745 | −0.10688 |
| EC.RBP2.ALPI | 0.03111 | 0.01461 | 0.00471 | 0.02043 | 0.01623 | 0.16820 | −0.10798 |
| Mac.CXCL2.CXCL3 | 0.02982 | −0.07411 | −0.10159 | −0.07708 | 0.00591 | −0.02694 | 0.06437 |
| EC.MT1H.PHGDH | 0.02935 | 0.01227 | 0.00182 | 0.02091 | 0.01150 | 0.16157 | −0.10541 |
| EC.GSTA2.AADAC | 0.02916 | 0.06185 | 0.01784 | 0.00636 | 0.00839 | 0.02655 | −0.07724 |
| NK.GNLY.IFNG | 0.02889 | −0.12894 | −0.04181 | −0.08078 | 0.03929 | 0.06819 | 0.07873 |
| Mono.CXCL3.FCN1 | 0.02666 | −0.15065 | −0.06615 | −0.09148 | 0.00839 | 0.00990 | 0.08193 |
| Mac.DC.CXCL10.CCL19 | 0.02661 | 0.01442 | −0.00263 | 0.02312 | 0.01057 | 0.16259 | −0.10793 |
| EC.GSTA2.TMPRSS15 | 0.02606 | 0.07109 | 0.02009 | −0.01417 | 0.00126 | 0.01226 | −0.09187 |
| T.GNLY.IGLC2 | 0.02584 | −0.10169 | −0.06429 | −0.06510 | 0.01536 | 0.08168 | 0.03894 |
| Mono.CXCL10.TNF | 0.02526 | −0.11257 | −0.09578 | −0.07823 | 0.03549 | 0.04465 | 0.07481 |
| EC.ADH1C.GSTA1 | 0.02504 | 0.06567 | 0.01281 | 0.00116 | 0.00755 | 0.01477 | −0.09951 |
| EC.ARHGDIG.SULT1E1 | 0.02451 | −0.02875 | 0.09072 | 0.17211 | −0.05329 | −0.00429 | 0.08632 |
| Goblet.S100P.TFF1 | 0.02416 | 0.01354 | −0.00118 | 0.02170 | 0.01137 | 0.16673 | −0.10993 |
| Mac.cDC1.MKI67.IDO1 | 0.02351 | −0.04579 | 0.04423 | 0.14634 | −0.10712 | −0.01424 | 0.13432 |
| NK.GNLY.GZMB | 0.02286 | −0.12419 | 0.04901 | −0.02115 | −0.03429 | −0.02601 | 0.01608 |
| Mono.S100A8.S100A9 | 0.02187 | −0.09127 | −0.09855 | −0.04285 | 0.00952 | −0.01251 | 0.07000 |
| Mono.FCN1.S100A4 | 0.02089 | −0.12171 | 0.01754 | −0.03119 | −0.04100 | 0.00511 | 0.02149 |
| EC.RBP2.CYP3A4 | 0.02022 | 0.09315 | 0.05144 | −0.02742 | 0.06689 | 0.00816 | −0.07846 |
| Mac.LYZ.TXN | 0.01953 | −0.09424 | 0.03702 | −0.02434 | −0.05602 | −0.02107 | 0.02446 |
| Mac.AIF1.HBEGF | 0.01891 | −0.09785 | 0.03736 | −0.02440 | −0.05613 | −0.02136 | 0.02531 |
| Mac.MKI67.APOE | 0.01813 | −0.07461 | 0.08307 | −0.06594 | −0.05154 | −0.01242 | −0.02952 |
| T.MAF.CTLA4 | 0.01771 | −0.16163 | 0.03087 | −0.03733 | −0.05006 | −0.05805 | −0.02291 |
| Mono.cDC2.FCN1.CD1C | 0.01672 | −0.07449 | −0.05313 | −0.08575 | 0.04018 | −0.01586 | 0.02215 |
| EC.LYZ.OLFM4 | 0.01643 | −0.08748 | −0.05834 | −0.06440 | 0.04067 | 0.07977 | 0.06139 |
| Prolif.EC.UBE2C.CCNB2 | 0.01615 | 0.01614 | 0.01825 | 0.02498 | 0.00462 | 0.16319 | −0.11630 |
| NK.GNLY.XCL1 | 0.01561 | 0.02998 | −0.06169 | −0.07857 | −0.09568 | −0.04640 | −0.12011 |
| EC.UBD.IFITM1 | 0.01556 | −0.01597 | −0.00690 | 0.00582 | 0.04542 | 0.05751 | −0.03136 |
| Mac.C1QB.AIF1 | 0.01494 | −0.09865 | 0.04052 | −0.02016 | −0.05826 | −0.02340 | 0.02183 |
| T.GNLY.CSF2 | 0.01466 | −0.13984 | −0.11800 | −0.06906 | 0.00150 | 0.02778 | 0.07128 |
| Goblet.BPIFB1.AQP5 | 0.01451 | −0.07581 | −0.05424 | −0.05966 | 0.03656 | 0.07541 | 0.04635 |
| Mono.Mac.CXCL10.GBP1 | 0.01373 | −0.07319 | −0.03734 | −0.02393 | 0.05053 | 0.06720 | −0.02588 |
| EC.MT1H.MT1G | 0.01370 | −0.05260 | −0.02402 | −0.09016 | 0.01910 | 0.13690 | 0.02760 |
| EC.FABP1.ADIRF | 0.01361 | 0.07205 | 0.10902 | −0.03097 | 0.00081 | 0.18042 | −0.07938 |
| T.MT.CO2.CCR7 | 0.01326 | 0.07484 | −0.01241 | 0.01343 | 0.00374 | 0.02052 | −0.10343 |
| T.TNFRSF4.MAF | 0.01309 | −0.07415 | 0.02549 | −0.00180 | −0.00056 | 0.02485 | −0.04152 |
| Mac.IGSF6.TXNIP | 0.01211 | −0.10103 | 0.04727 | −0.02246 | −0.06082 | −0.02017 | 0.02005 |
| Enteroendocrine.ONECUT3.CCK | 0.00730 | −0.03072 | 0.00191 | 0.12491 | −0.01492 | 0.17983 | −0.08047 |
| T.TNFRSF18.FOXP3 | 0.00694 | −0.08926 | −0.06827 | −0.09388 | 0.03155 | 0.11391 | −0.00509 |
| T.MKI67.IFNG | 0.00598 | −0.13652 | −0.10254 | −0.04973 | 0.00828 | 0.01873 | 0.04549 |
| T.MKI67.CD38 | 0.00589 | 0.02959 | 0.05459 | 0.09108 | −0.07865 | −0.02812 | 0.05062 |
| EC.CA1.TMPRSS15 | 0.00574 | −0.05611 | −0.06223 | −0.03160 | 0.09894 | 0.07718 | 0.00936 |
| Mac.DC.CXCL10.CLEC4E | 0.00515 | −0.13362 | 0.05478 | −0.01438 | −0.05895 | −0.02149 | 0.00548 |
| Mac.CXCL3.APOC1 | 0.00181 | −0.14070 | −0.02359 | −0.04479 | 0.02392 | 0.05275 | 0.03147 |
| Mac.C1QB.CD14 | −0.00084 | −0.15374 | 0.01625 | −0.04884 | −0.00810 | 0.03713 | 0.03339 |
| Goblet.HES6.COLCA2 | −0.00150 | −0.08634 | 0.13525 | 0.06037 | −0.04460 | 0.11874 | −0.07183 |
| T.MKI67.FOXP3 | −0.00261 | −0.13820 | −0.10018 | −0.01412 | −0.01900 | 0.05494 | 0.02371 |
| Goblet.RETNLB.ITLN1 | −0.00603 | −0.15479 | −0.01251 | −0.04155 | −0.01400 | 0.06790 | 0.02859 |
| DC.CXCL10.IDO1 | −0.00833 | −0.00325 | 0.05098 | 0.01599 | 0.20503 | −0.06621 | −0.02125 |
| T.MKI67.IL22 | −0.00882 | −0.13262 | −0.04476 | −0.01918 | −0.07334 | −0.03289 | 0.01859 |
| EC.ANPEP.DUOX2 | −0.01534 | −0.02183 | 0.05824 | 0.01093 | 0.03125 | 0.09528 | −0.07045 |
| cDC2.CD1C.AREG | −0.02054 | −0.16689 | 0.09611 | 0.03843 | −0.02106 | −0.04146 | −0.03199 |
| EC.ADH1C.RPS4Y1 | −0.02062 | 0.07479 | 0.01736 | −0.00721 | −0.02718 | −0.01948 | −0.06955 |
| Mono.FCN1.LYST | −0.02562 | −0.10761 | 0.11347 | −0.03933 | −0.08169 | 0.04298 | −0.01881 |
| Goblet.ITLN1.CLCA1.1 | −0.02723 | −0.08920 | 0.13646 | 0.12368 | −0.07627 | −0.04655 | −0.00598 |
| EC.APOC3.APOA4 | −0.02851 | 0.02797 | 0.03407 | 0.00600 | 0.22477 | −0.08084 | 0.05231 |
| EC.APOC3.CUBN | −0.02991 | 0.02551 | 0.03304 | 0.00745 | 0.22424 | −0.08646 | 0.05560 |
| EC.CFTR.OLFM4 | −0.03007 | 0.02509 | 0.03304 | 0.00747 | 0.22438 | −0.08624 | 0.05587 |
| EC.FABP6.SLC26A3 | −0.03023 | 0.02542 | 0.03326 | 0.00697 | 0.22393 | −0.08634 | 0.05635 |
| EC.OLFM4.MT.ND2 | −0.03030 | −0.12852 | 0.04059 | −0.09586 | −0.05403 | 0.08017 | 0.08266 |
| Goblet.FCGBP.CLCA1 | −0.03105 | 0.02614 | 0.03304 | 0.00622 | 0.22352 | −0.08596 | 0.05710 |
| EC.PLCG2.MTRNR2L8 | −0.03118 | 0.02627 | 0.03327 | 0.00624 | 0.22348 | −0.08627 | 0.05706 |
| EC.MIF.MTRNR2L8 | −0.03208 | 0.02602 | 0.03210 | 0.00658 | 0.22336 | −0.08584 | 0.05712 |
| EC.APOB.APOC3 | −0.03234 | 0.03013 | 0.03858 | −0.00078 | 0.22157 | −0.08352 | 0.06042 |
| T.CCL20.RORA | −0.03365 | −0.16057 | 0.08105 | 0.02212 | −0.05798 | −0.06605 | −0.02912 |
| EC.NUPR1.LCN2 | −0.03437 | −0.14010 | 0.09686 | 0.03549 | −0.06753 | −0.04760 | −0.03277 |
| T.MKI67.MT.CO2 | −0.03749 | −0.03299 | −0.09921 | 0.08538 | 0.03787 | 0.09122 | −0.03337 |
| Goblet.ITLN1.CLCA1 | −0.03985 | −0.05495 | 0.14123 | 0.08879 | −0.03628 | −0.01440 | −0.07415 |
| NK.GNLY.FCER1G | −0.04137 | −0.13308 | 0.11131 | 0.04030 | −0.03959 | −0.01782 | −0.08889 |
| EC.FABP6.PLCG2 | −0.04276 | 0.08020 | 0.12149 | −0.12081 | −0.03378 | 0.04776 | 0.05589 |
| Secretory.GSTA1.REG1B | −0.04463 | 0.06633 | 0.10096 | −0.14288 | −0.06547 | 0.05426 | 0.09268 |
| Mac.CXCL8.HES1 | −0.04589 | 0.06910 | 0.09981 | −0.14484 | −0.05808 | 0.04856 | 0.08936 |
| Mono.Mac.DC.CXCL8.CLEC10A | −0.04647 | 0.06110 | 0.09065 | −0.15138 | −0.07221 | 0.04786 | 0.07433 |
| cDC2.CD1C.CD207 | −0.04709 | 0.06838 | 0.09913 | −0.14293 | −0.06236 | 0.05308 | 0.09184 |
| Mono.cDC2.CLEC10A.SDS | −0.04709 | 0.06838 | 0.09913 | −0.14293 | −0.06236 | 0.05308 | 0.09184 |
| EC.ADH1C.EDN1 | −0.04871 | 0.08372 | 0.10144 | −0.14492 | −0.06707 | 0.05774 | 0.07212 |
| pDC.IRF7.IL3RA | −0.05091 | −0.15064 | −0.06219 | 0.06931 | 0.04092 | −0.00061 | 0.01700 |
| EC.GNAT3.TRPM5 | −0.05348 | 0.09612 | 0.11569 | −0.11458 | 0.06311 | 0.00484 | 0.07432 |
| cDC2.FCER1A.CD1C | −0.05457 | 0.03235 | 0.00772 | −0.00253 | −0.03586 | −0.02940 | 0.00430 |
| T.CTLA4.IL2RA | −0.05457 | −0.09561 | 0.12380 | 0.07372 | −0.02247 | −0.04217 | −0.08527 |
| Secretory.REG1B.REG1A | −0.05670 | −0.10119 | 0.14552 | 0.06143 | −0.06513 | −0.03969 | −0.05275 |
| Mono.FCN1.AREG | −0.05726 | −0.09886 | 0.12458 | 0.07618 | −0.02790 | −0.05111 | −0.07854 |
| cDC2.MKI67.CD1C | −0.05733 | 0.05944 | 0.01732 | −0.07965 | −0.10858 | −0.07450 | 0.01251 |
| EC.HSPA1B.PSMA2 | −0.05803 | −0.08953 | 0.12274 | 0.07950 | −0.03044 | −0.05026 | −0.08289 |
| EC.SMOC2.ASCL2 | −0.05983 | −0.08911 | 0.12400 | 0.08015 | −0.03081 | −0.05125 | −0.08384 |
| EC.GSTA2.CES3 | −0.06048 | 0.04825 | 0.02707 | −0.03475 | −0.04606 | −0.01425 | 0.01780 |
| T.GZMA.JAML | −0.06179 | −0.05960 | 0.10872 | 0.06040 | 0.09040 | −0.11050 | −0.04978 |
| EC.TMPRSS15.FAM8A1 | −0.06292 | −0.08991 | 0.12617 | 0.07754 | 0.02658 | −0.06117 | −0.06922 |
| Goblet.FCGBP.HES6 | −0.06460 | 0.08392 | 0.10331 | −0.12415 | −0.08035 | 0.04580 | 0.09168 |
| Prolif.Secretory.REG1B.MGST1 | −0.06526 | −0.10705 | 0.12555 | 0.07794 | −0.05459 | −0.04629 | −0.05770 |
| ILC.AREG.AHR | −0.06993 | −0.07733 | 0.12661 | 0.08154 | 0.04874 | −0.07606 | −0.06251 |
| EC.PLCG2.RP11.727F159 | −0.07215 | 0.06240 | 0.11491 | −0.10752 | −0.08064 | 0.07096 | 0.06868 |
| Goblet.CENPA.UBE2C | −0.07415 | −0.07782 | 0.16598 | 0.06914 | −0.01368 | −0.02701 | −0.03832 |
| Goblet.FCGBP.ITLN1 | −0.07555 | 0.07243 | 0.10708 | −0.11563 | −0.08918 | 0.03350 | 0.07223 |
| Enteroendocrine.TFPI2.TPH1 | −0.07633 | 0.00347 | −0.05328 | 0.09304 | 0.09893 | 0.07534 | 0.04195 |
| cDC2.CLEC10A.FCGR2B | −0.07814 | 0.09073 | 0.04370 | −0.12409 | −0.08514 | 0.03961 | 0.06364 |
| Paneth.DEFA6.ITLN2 | −0.08845 | 0.03285 | 0.12944 | −0.03397 | 0.13815 | −0.02719 | 0.04700 |
| Myeloid.MKI67.IGKC | −0.09011 | 0.08480 | 0.05916 | −0.09357 | −0.06764 | −0.02326 | 0.05214 |
| EC.MTRNR2L1.MT.ND3 | −0.09634 | 0.07623 | 0.09797 | −0.12624 | −0.03566 | 0.05963 | 0.07062 |
| EC.PLCG2.MAFB | −0.10130 | 0.06050 | 0.07954 | −0.10665 | −0.06958 | 0.06086 | 0.07625 |
| T.GNLY.MT.CO2 | −0.10804 | −0.06736 | 0.08827 | 0.06996 | −0.05670 | −0.05665 | −0.06554 |
| T.MT.CO2.MT.CO1 | −0.10946 | 0.10483 | −0.03235 | −0.03463 | −0.08121 | −0.01468 | −0.06191 |
| Goblet.TFF1.TPSG1 | −0.11095 | −0.02579 | −0.16028 | 0.01836 | −0.02570 | 0.04478 | 0.02221 |
| EC.MIF.AC0904981 | −0.11980 | 0.03549 | −0.06240 | 0.03829 | 0.16066 | −0.05088 | 0.02283 |
| Goblet.FCGBP.SPINK4 | −0.12042 | 0.03526 | −0.01183 | −0.01674 | −0.06715 | 0.01328 | 0.00673 |
| Enteroendocrine.NEUROG3.MLN | −0.12438 | −0.04891 | −0.05951 | 0.09888 | −0.05303 | 0.03023 | −0.05939 |
| T.CTLA4.CD27 | −0.12464 | 0.03398 | −0.11252 | 0.05654 | −0.02455 | −0.00203 | −0.05194 |
| DC.CCR7.FSCN1 | −0.12487 | 0.00424 | −0.02399 | −0.06063 | 0.12525 | −0.07200 | 0.06399 |
| NK.MKI67.GZMA | −0.12659 | −0.11073 | −0.01864 | 0.06922 | 0.04323 | −0.07876 | −0.00735 |
| NK.CCL3.CD160 | −0.12721 | 0.00399 | −0.00253 | 0.02580 | −0.01769 | −0.03428 | −0.01702 |
| cDC2.RALA.CD1C | −0.13242 | 0.02458 | −0.13357 | 0.05624 | −0.04885 | 0.00342 | −0.00203 |
| cDC2.ZFAND2A.INHBA | −0.13242 | 0.02458 | −0.13357 | 0.05624 | −0.04885 | 0.00342 | −0.00203 |
| T.NR3C1.CCR6 | −0.13343 | 0.02584 | −0.13195 | 0.05374 | −0.05003 | 0.00437 | −0.00039 |
| T.GNLY.HOPX | −0.13352 | 0.02415 | −0.13490 | 0.05484 | −0.04967 | 0.00297 | −0.00111 |
| Mono.Mac.CCL4.DDX3Y | −0.13408 | 0.02577 | −0.13243 | 0.05670 | −0.03897 | −0.00043 | 0.00047 |
| Mono.FCN1.HSPA1A | −0.13427 | 0.02488 | −0.13481 | 0.05380 | −0.05042 | 0.00271 | 0.00024 |
| T.TIGIT.CTLA4 | −0.13442 | 0.02568 | −0.13305 | 0.05025 | −0.05124 | 0.00512 | 0.00201 |
| Mono.Mac.DC.CXCL10.IFIT2 | −0.13629 | 0.02529 | −0.13617 | 0.05477 | −0.05146 | 0.00062 | −0.00007 |
| TA.PLCG2.SOX4 | −0.13787 | −0.03778 | −0.05581 | 0.00371 | −0.05462 | 0.08090 | −0.01442 |
| DC.LTB.IL22RA2 | −0.13857 | 0.05446 | −0.02606 | −0.06090 | −0.13329 | −0.00438 | −0.02730 |
| T.MT.CO2.MT.CO3 | −0.13987 | −0.01004 | 0.01562 | 0.06037 | −0.07896 | −0.00281 | −0.08328 |
| T.B2M.MT.CO2 | −0.14113 | −0.02253 | −0.10995 | 0.06240 | −0.04406 | 0.01230 | −0.03483 |
| NK.GNLY.FCGR3A | −0.14150 | 0.03042 | −0.13497 | 0.05373 | −0.05538 | −0.00299 | −0.00180 |
| Prolif.EC.UBE2C.HMGB2 | −0.14297 | −0.02196 | 0.03903 | −0.02202 | 0.02523 | 0.04449 | −0.01250 |
| T.MT.CO2.PTGER4 | −0.14298 | 0.06087 | −0.09401 | 0.02386 | −0.06895 | −0.00587 | −0.02272 |
| Enteroendocrine.TPH1.CHGA | −0.14615 | 0.05545 | 0.04902 | −0.09685 | 0.00406 | 0.06528 | 0.08308 |
| cDC1.CLEC9A.IDO1 | −0.14656 | 0.01684 | −0.00914 | 0.02549 | 0.02168 | −0.06437 | −0.02835 |
| Myeloid.MKI67.PCNA | −0.14689 | 0.02138 | −0.01074 | 0.00273 | 0.04206 | −0.10778 | 0.02782 |
| Mac.JUN.EGR1 | −0.14963 | 0.03902 | −0.12480 | 0.04690 | −0.04904 | −0.00152 | 0.00393 |
| Mono.FCN1.EIF4A3 | −0.15371 | 0.04560 | −0.11014 | 0.03120 | −0.06991 | −0.00039 | 0.01188 |
| Goblet.REG4.SPINK4 | −0.17178 | −0.03924 | −0.03637 | 0.05295 | −0.06169 | −0.01241 | 0.00112 |
| cDC1.CLEC9A.XCR1 | −0.17389 | −0.03073 | −0.00920 | 0.01237 | −0.06020 | −0.00551 | −0.02181 |
| | PC8 | PC9 | PC10 | PC11 | PC12 | PC13 | PC14 |
| variance | 0.06206 | 0.05731 | 0.05161 | 0.04485 | 0.03908 | 0.03648 | 0.00000 |
| T.CCR7.GPR183 | −0.03192 | −0.01250 | 0.00904 | 0.00298 | 0.03087 | −0.01494 | −0.02134 |
| T.MKI67.RRM2 | 0.12139 | 0.06024 | 0.04157 | −0.13777 | 0.03199 | −0.04225 | 0.00004 |
| cDC2.CLEC10A.CD1E | 0.00896 | −0.07187 | −0.01315 | 0.00288 | 0.08386 | −0.10360 | 0.21201 |
| cDC2.CD1C.NDRG2 | 0.01542 | 0.03652 | 0.03542 | −0.05443 | −0.01830 | −0.05283 | 0.10267 |
| T.GNLY.GZMH | 0.02441 | −0.01691 | 0.01358 | 0.09480 | −0.03606 | 0.01675 | 0.00342 |
| T.MKI67.MAF | 0.00792 | 0.01939 | −0.10638 | 0.03188 | −0.09181 | −0.07191 | −0.04897 |
| T.CCL20.IFNG | 0.01251 | 0.06594 | 0.01748 | 0.09828 | −0.12161 | 0.05198 | −0.01365 |
| T.CCL4.GMZK | 0.01111 | 0.02601 | 0.01595 | −0.01410 | −0.02052 | 0.05573 | 0.02949 |
| T.IFI44L.PTGER4 | −0.09849 | −0.03901 | 0.03764 | 0.03200 | −0.12662 | 0.02934 | −0.02194 |
| T.GNLY.CTSW | 0.06744 | 0.11563 | 0.02964 | −0.00786 | −0.02283 | 0.10492 | −0.02588 |
| T.CD8B.TRGC2 | −0.04030 | 0.07885 | 0.03180 | 0.09800 | −0.07418 | −0.01705 | −0.02139 |
| T.GNLY.IFNG | 0.06053 | 0.09784 | 0.03369 | −0.02508 | 0.00298 | 0.09794 | −0.05661 |
| Mcell.CCL23.SPIB | −0.03337 | 0.00081 | −0.01493 | 0.00229 | −0.04480 | 0.02581 | −0.00005 |
| T.KLRB1.LTF | 0.01177 | 0.09395 | 0.03880 | −0.00778 | −0.00702 | 0.05886 | −0.01116 |
| T.LAG3.BATF | −0.02738 | −0.05598 | 0.02514 | −0.12877 | 0.10539 | −0.23476 | −0.01235 |
| Mono.cDC2.CLEC10A.AREG | 0.03882 | 0.09205 | 0.04155 | −0.01152 | −0.01407 | 0.08482 | −0.00011 |
| T.MKI67.ITGB7 | 0.00565 | 0.04309 | 0.01293 | −0.00174 | −0.02206 | 0.03228 | −0.00098 |
| Mac.DC.CXCL8.NLRP3 | 0.02716 | 0.11745 | 0.03156 | 0.04463 | 0.00234 | 0.03483 | −0.00932 |
| EC.IFI6.IFI27 | −0.02605 | 0.02136 | 0.00381 | −0.01993 | −0.04160 | 0.03846 | 0.00445 |
| ILC.KRT81.IL22 | −0.01358 | 0.02876 | 0.01498 | 0.00471 | −0.00912 | 0.02413 | 0.00465 |
| T.CCL20.IL22 | −0.01333 | 0.02704 | 0.01255 | −0.00237 | −0.01607 | 0.02065 | −0.00337 |
| T.IFI6.IRF7 | −0.01913 | 0.02167 | 0.00398 | −0.00632 | −0.02130 | 0.02420 | 0.01732 |
| Secretory.REG3G.IFI6 | −0.01244 | 0.02560 | 0.01299 | −0.00561 | −0.01555 | 0.02318 | 0.00532 |
| EC.TCL1A.IFI6 | −0.01433 | 0.02630 | 0.01657 | −0.00870 | −0.01247 | 0.01989 | −0.02235 |
| T.TRDC.TRGC2 | −0.03248 | 0.03555 | 0.01116 | −0.18858 | −0.02065 | 0.07897 | 0.02470 |
| T.CCR7.SELL | −0.05418 | −0.07305 | −0.04420 | −0.08951 | 0.14746 | −0.11395 | −0.04146 |
| Mac.FOLR2.LILRB5 | 0.05259 | 0.10487 | 0.05032 | −0.02273 | 0.01190 | 0.09970 | −0.00139 |
| EC.SLC28A2.GSTA2 | −0.04199 | −0.08707 | 0.01466 | −0.13461 | 0.09745 | −0.24160 | −0.00219 |
| ILC.LST1.AREG | 0.04958 | 0.11112 | 0.08709 | −0.00612 | 0.02977 | 0.10770 | 0.01340 |
| T.MKI67.CCR7 | 0.01516 | −0.03772 | 0.01041 | −0.13234 | 0.13109 | −0.25509 | −0.00416 |
| ILC.IL22.KIT | −0.01888 | −0.06737 | 0.01177 | −0.14148 | 0.12937 | −0.25015 | −0.04465 |
| T.CARD16.GB2 | 0.00535 | −0.13931 | 0.03358 | 0.05615 | 0.10421 | 0.12561 | −0.03474 |
| T.MKI67.NKG7 | −0.01925 | 0.01351 | −0.01462 | 0.02972 | −0.00844 | 0.04317 | 0.03647 |
| T.EGR1.TNF | −0.01247 | 0.08319 | 0.00412 | −0.03317 | −0.04859 | 0.10118 | 0.01827 |
| T.EGR1.IFNG | −0.02825 | −0.09530 | 0.04955 | 0.09506 | −0.24047 | 0.00523 | 0.01469 |
| Mono.Mac.CXCL10.FCN1 | −0.05550 | −0.00843 | −0.01353 | 0.13292 | 0.02092 | −0.06893 | −0.01661 |
| Mac.APOE.PTGDS | 0.14127 | −0.07519 | 0.05259 | 0.01321 | −0.00920 | 0.01126 | −0.02386 |
| NK.GNLY.KLRC1 | −0.11276 | 0.06507 | −0.02002 | −0.09174 | −0.03581 | 0.03249 | 0.01895 |
| Mcell.CSRP2.SPIB | −0.01654 | −0.04072 | 0.04612 | −0.12988 | 0.13688 | −0.24771 | −0.00582 |
| Mac.SEPP1.CXCL3 | 0.00053 | 0.14194 | 0.05500 | 0.09534 | −0.02846 | −0.03791 | −0.00180 |
| Mac.C1QB.FCGR1A | −0.01707 | 0.04216 | −0.00034 | 0.22440 | 0.16662 | 0.06975 | −0.00359 |
| Mono.Mac.CXCL10.LYZ | −0.09777 | 0.05267 | −0.02484 | 0.19546 | 0.03083 | −0.12225 | 0.01827 |
| Goblet.S100P.FCGBP | 0.14492 | 0.08961 | −0.03669 | 0.05181 | −0.05150 | 0.06196 | −0.01099 |
| Mac.CXCL2.TNF | −0.06614 | 0.05295 | −0.03740 | 0.19638 | −0.03147 | −0.18199 | −0.02851 |
| EC.LECT1.FAM26F | −0.14299 | −0.12573 | 0.02574 | 0.03937 | −0.21345 | 0.05515 | 0.00694 |
| EpithStem.LINC00176.RPS4Y1 | −0.13028 | −0.12335 | 0.02016 | 0.03946 | −0.22176 | 0.03685 | 0.00086 |
| Mono.Mac.CXCL10.CXCL11 | −0.08648 | 0.04425 | −0.02280 | −0.00963 | −0.03597 | −0.05799 | −0.00873 |
| EC.AKR1C1.TSPAN1 | 0.14655 | 0.11035 | −0.03319 | 0.04761 | −0.09003 | 0.02335 | −0.00990 |
| EC.RBP2.ALPI | 0.15022 | 0.10796 | −0.02635 | 0.06825 | −0.05611 | 0.02509 | 0.01276 |
| Mac.CXCL2.CXCL3 | −0.07452 | 0.07855 | −0.01593 | 0.14847 | −0.03079 | −0.16221 | −0.00078 |
| EC.MT1H.PHGDH | 0.15656 | 0.11191 | −0.03297 | 0.06012 | −0.07651 | 0.01463 | −0.00950 |
| EC.GSTA2.AADAC | −0.15928 | −0.13357 | 0.03704 | 0.06790 | −0.17785 | 0.09259 | −0.01667 |
| NK.GNLY.IFNG | −0.05537 | −0.00506 | 0.01750 | −0.14957 | −0.02487 | 0.06852 | 0.00893 |
| Mono.CXCL3.FCN1 | −0.04140 | 0.00007 | −0.04105 | 0.00312 | −0.06202 | −0.07251 | 0.02373 |
| Mac.DC.CXCL10.CCL19 | 0.15791 | 0.10994 | −0.02962 | 0.05610 | −0.07321 | 0.00755 | −0.00823 |
| EC.GSTA2.TMPRSS15 | −0.14534 | −0.12837 | 0.03293 | 0.03312 | −0.21027 | 0.03297 | −0.01432 |
| T.GNLY.IGLC2 | −0.01851 | 0.04164 | −0.01743 | −0.20688 | −0.08100 | 0.08402 | 0.02322 |
| Mono.CXCL10.TNF | −0.09965 | 0.07466 | −0.03791 | −0.08596 | −0.06424 | −0.02557 | 0.00655 |
| EC.ADH1C.GSTA1 | −0.14398 | −0.13488 | 0.00388 | 0.03339 | −0.21143 | 0.03648 | −0.01182 |
| EC.ARHGDIG.SULT1E1 | −0.07475 | 0.07041 | 0.04537 | 0.02021 | 0.05281 | 0.03213 | −0.00681 |
| Goblet.S100P.TFF1 | 0.15174 | 0.10321 | −0.05419 | 0.06582 | −0.05214 | 0.03417 | 0.03290 |
| Mac.cDC1.MKI67.IDO1 | 0.03126 | −0.00135 | −0.08413 | 0.04640 | −0.00917 | −0.07799 | 0.02791 |
| NK.GNLY.GZMB | 0.10772 | −0.18862 | −0.00797 | −0.00368 | −0.05296 | 0.00880 | 0.03433 |
| Mono.S100A8.S100A9 | −0.06744 | 0.05069 | −0.02340 | 0.16965 | −0.03511 | −0.16620 | 0.02958 |
| Mono.FCN1.S100A4 | 0.11891 | −0.19092 | 0.02889 | 0.03410 | 0.02385 | 0.03658 | 0.00191 |
| EC.RBP2.CYP3A4 | −0.14137 | −0.13234 | 0.06637 | 0.02964 | −0.12936 | 0.00994 | −0.00769 |
| Mac.LYZ.TXN | 0.14587 | −0.20365 | 0.01919 | 0.00010 | −0.02797 | 0.00745 | −0.02505 |
| Mac.AIF1.HBEGF | 0.14508 | −0.20002 | 0.01897 | −0.00127 | −0.02659 | 0.00532 | −0.00407 |
| Mac.MKI67.APOE | 0.18797 | −0.09748 | −0.04391 | 0.06919 | −0.08331 | 0.01248 | −0.05016 |
| T.MAF.CTLA4 | −0.06386 | −0.02458 | 0.00483 | 0.08938 | −0.02663 | −0.11094 | −0.01430 |
| Mono.cDC2.FCN1.CD1C | −0.08823 | −0.00273 | −0.24194 | −0.00843 | −0.03493 | 0.01977 | 0.00885 |
| EC.LYZ.OLFM4 | −0.07661 | 0.05762 | −0.00807 | −0.20622 | −0.04012 | 0.09336 | 0.02697 |
| Prolif.EC.UBE2C.CCNB2 | 0.13702 | 0.11467 | −0.06635 | 0.05084 | −0.07951 | 0.01516 | −0.00111 |
| NK.GNLY.XCL1 | 0.11139 | 0.11668 | −0.09675 | 0.05822 | −0.04927 | 0.08556 | 0.04626 |
| EC.UBD.IFITM1 | −0.07193 | −0.04393 | 0.05069 | 0.10906 | 0.25959 | 0.18855 | 0.01565 |
| Mac.C1QB.AIF1 | 0.14500 | −0.19878 | 0.02190 | −0.00095 | −0.02595 | 0.00413 | −0.01421 |
| T.GNLY.CSF2 | −0.04591 | 0.00726 | 0.00035 | −0.01038 | −0.07768 | −0.05426 | 0.02269 |
| Goblet.BPIFB1.AQP5 | −0.06967 | 0.05715 | −0.01578 | −0.23380 | −0.05462 | 0.09737 | 0.00642 |
| Mono.Mac.CXCL10.GBP1 | −0.08233 | −0.05076 | 0.06811 | 0.09452 | 0.20699 | 0.17613 | −0.01065 |
| EC.MT1H.MT1G | −0.01018 | 0.07124 | −0.02607 | −0.21694 | −0.06967 | 0.07569 | 0.04587 |
| EC.FABP1.ADIRF | 0.00048 | 0.06435 | 0.04715 | 0.05265 | −0.06767 | 0.00670 | 0.01140 |
| T.MT.CO2.CCR7 | −0.15750 | −0.13788 | 0.06129 | 0.00527 | −0.17166 | 0.00046 | −0.03505 |
| T.TNFRSF4.MAF | −0.11878 | −0.04723 | 0.02004 | 0.12640 | 0.22625 | 0.14924 | −0.00304 |
| Mac.IGSF6.TXNIP | 0.14166 | −0.19449 | 0.02553 | −0.00386 | −0.03080 | 0.00362 | 0.01302 |
| Enteroendocrine.ONECUT3.CCK | 0.05907 | 0.09094 | 0.05613 | 0.00256 | 0.03412 | 0.06664 | −0.00464 |
| T.TNFRSF18.FOXP3 | −0.05640 | 0.08505 | −0.08214 | 0.14223 | −0.03698 | −0.06270 | −0.01682 |
| T.MKI67.IFNG | −0.08164 | 0.03182 | 0.00184 | 0.11440 | −0.01404 | −0.10180 | 0.01854 |
| T.MKI67.CD38 | −0.02021 | −0.10390 | −0.23697 | −0.03630 | 0.00141 | −0.01187 | −0.00097 |
| EC.CA1.TMPRSS15 | −0.11416 | −0.01347 | 0.04758 | −0.02115 | 0.15409 | 0.20376 | −0.02418 |
| Mac.DC.CXCL10.CLEC4E | 0.10734 | −0.17001 | 0.02201 | −0.00345 | −0.01405 | 0.01174 | −0.01982 |
| Mac.CXCL3.APOC1 | −0.05684 | 0.05706 | −0.00505 | −0.18578 | −0.07554 | 0.03818 | −0.00987 |
| Mac.C1QB.CD14 | −0.03514 | −0.00354 | 0.01339 | −0.18367 | −0.05136 | 0.05167 | 0.02456 |
| Goblet.HES6.COLCA2 | 0.03230 | 0.07160 | 0.05669 | 0.03372 | −0.01658 | 0.06186 | −0.00866 |
| T.MKI67.FOXP3 | 0.01135 | 0.01576 | 0.03264 | 0.14187 | −0.02357 | −0.12100 | −0.06478 |
| Goblet.RETNLB.ITLN1 | −0.06172 | 0.05949 | 0.02300 | −0.13039 | −0.09565 | 0.01460 | 0.03309 |
| DC.CXCL10.IDO1 | −0.01444 | −0.07437 | 0.00874 | 0.07874 | 0.05682 | 0.14699 | 0.16695 |
| T.MKI67.IL22 | 0.10122 | −0.16149 | 0.03663 | 0.01790 | −0.00781 | 0.04955 | −0.01881 |
| EC.ANPEP.DUOX2 | −0.11568 | −0.02046 | 0.07324 | 0.10125 | 0.15777 | 0.20063 | 0.01053 |
| cDC2.CD1C.AREG | −0.02352 | −0.03185 | 0.04663 | 0.02802 | 0.00229 | −0.01998 | 0.06765 |
| EC.ADH1C.RPS4Y1 | −0.10132 | −0.10556 | −0.22135 | 0.01021 | −0.10009 | 0.04718 | −0.00369 |
| Mono.FCN1.LYST | 0.11687 | −0.08904 | −0.04756 | −0.03758 | −0.06805 | 0.01544 | −0.02021 |
| Goblet.ITLN1.CLCA1.1 | −0.05518 | −0.00151 | −0.03614 | −0.01138 | −0.03207 | −0.00113 | 0.00931 |
| EC.APOC3.APOA4 | 0.07971 | 0.00987 | 0.01741 | 0.00335 | −0.04023 | 0.00415 | 0.00482 |
| EC.APOC3.CUBN | 0.07334 | 0.00568 | 0.01717 | 0.00335 | −0.04050 | 0.00761 | −0.00607 |
| EC.CFTR.OLFM4 | 0.07338 | 0.00602 | 0.01737 | 0.00423 | −0.03887 | 0.00914 | −0.00984 |
| EC.FABP6.SLC26A3 | 0.07376 | 0.00630 | 0.01730 | 0.00368 | −0.04020 | 0.00807 | −0.02307 |
| EC.OLFM4.MT.ND2 | 0.06795 | 0.00862 | −0.05382 | −0.05396 | −0.08587 | −0.02008 | −0.00784 |
| Goblet.FCGBP.CLCA1 | 0.07405 | 0.00624 | 0.01821 | 0.00375 | −0.04014 | 0.00788 | −0.01335 |
| EC.PLCG2.MTRNR2L8 | 0.07398 | 0.00607 | 0.01564 | 0.00371 | −0.03980 | 0.00826 | 0.01044 |
| EC.MIF.MTRNR2L8 | 0.07461 | 0.00521 | 0.01937 | 0.00413 | −0.03920 | 0.00858 | 0.01499 |
| EC.APOB.APOC3 | 0.07213 | 0.00659 | 0.02186 | 0.00477 | −0.04125 | 0.00586 | 0.01611 |
| T.CCL20.RORA | 0.02096 | −0.06641 | 0.03376 | 0.03203 | −0.00408 | −0.01813 | 0.04895 |
| EC.NUPR1.LCN2 | 0.06491 | −0.09216 | 0.04536 | −0.01527 | −0.02810 | −0.03423 | 0.00171 |
| T.MKI67.MT.CO2 | 0.06438 | 0.08518 | −0.13572 | 0.08371 | −0.07186 | −0.12307 | −0.04969 |
| Goblet.ITLN1.CLCA1 | −0.13558 | 0.00371 | −0.00172 | 0.06123 | 0.06040 | 0.06461 | −0.01013 |
| NK.GNLY.FCER1G | −0.06713 | 0.08306 | 0.01826 | −0.01932 | −0.00366 | 0.00238 | 0.01301 |
| EC.FABP6.PLCG2 | −0.07035 | 0.00056 | 0.09728 | 0.03971 | −0.05574 | −0.00813 | 0.03770 |
| Secretory.GSTA1.REG1B | −0.00060 | 0.03210 | 0.08105 | 0.01968 | 0.01674 | −0.03928 | 0.03049 |
| Mac.CXCL8.HES1 | −0.00748 | 0.04253 | 0.08158 | 0.02550 | 0.01116 | −0.03922 | 0.01991 |
| Mono.Mac.DC.CXCL8.CLEC10A | −0.01873 | 0.02098 | 0.10710 | −0.00304 | 0.03517 | −0.01382 | 0.03874 |
| cDC2.CD1C.CD207 | −0.00858 | 0.03757 | 0.07996 | 0.01999 | 0.01777 | −0.03849 | −0.18322 |
| Mono.cDC2.CLEC10A.SDS | −0.00858 | 0.03757 | 0.07996 | 0.01999 | 0.01777 | −0.03849 | 0.02612 |
| EC.ADH1C.EDN1 | −0.00902 | 0.03291 | 0.03055 | 0.02652 | −0.00086 | −0.02821 | 0.01830 |
| pDC.IRF7.IL3RA | 0.02949 | 0.01267 | −0.02917 | 0.13777 | −0.01957 | −0.01585 | −0.01283 |
| EC.GNAT3.TRPM5 | −0.01892 | −0.00542 | 0.05905 | 0.03223 | −0.07963 | −0.01412 | 0.02550 |
| cDC2.FCER1A.CD1C | −0.00580 | −0.02422 | −0.30029 | −0.00669 | 0.04706 | 0.04024 | 0.18136 |
| T.CTLA4.IL2RA | −0.09038 | 0.08569 | 0.04555 | 0.00153 | 0.01373 | −0.03433 | 0.02228 |
| Secretory.REG1B.REG1A | −0.01814 | 0.04675 | 0.05780 | −0.00584 | −0.01326 | −0.06302 | 0.01138 |
| Mono.FCN1.AREG | −0.06516 | 0.08732 | 0.03516 | −0.01769 | −0.01015 | −0.05366 | 0.01749 |
| cDC2.MKI67.CD1C | 0.00582 | 0.03491 | −0.20594 | 0.04385 | 0.05594 | 0.05761 | 0.15047 |
| EC.HSPA1B.PSMA2 | −0.07323 | 0.09784 | 0.03389 | −0.00839 | −0.00542 | −0.05776 | 0.02450 |
| EC.SMOC2.ASCL2 | −0.07372 | 0.08820 | 0.03824 | −0.00681 | −0.01520 | −0.05615 | 0.00480 |
| EC.GSTA2.CES3 | −0.02004 | −0.02362 | −0.28416 | −0.01432 | 0.03934 | 0.03258 | 0.04009 |
| T.GZMA.JAML | −0.09193 | 0.08487 | 0.01022 | 0.01487 | −0.08189 | −0.01581 | −0.00004 |
| EC.TMPRSS15.FAM8A1 | −0.06637 | 0.09958 | 0.04048 | −0.01506 | 0.00080 | −0.03581 | 0.01490 |
| Goblet.FCGBP.HES6 | −0.02111 | 0.02560 | −0.02459 | 0.02130 | 0.01242 | −0.01894 | 0.03718 |
| Prolif.Secretory.REG1B.MGST1 | −0.02981 | 0.05180 | 0.05543 | −0.01140 | −0.01707 | −0.05802 | 0.01144 |
| ILC.AREG.AHR | −0.04361 | 0.09672 | 0.04224 | −0.00446 | −0.01558 | −0.04985 | 0.00094 |
| EC.PLCG2.RP11.727F159 | −0.00592 | 0.04860 | 0.08127 | 0.03753 | −0.00800 | −0.02999 | 0.03058 |
| Goblet.CENPA.UBE2C | 0.00799 | 0.06937 | −0.04162 | 0.01583 | −0.03350 | −0.03116 | 0.05435 |
| Goblet.FCGBP.ITLN1 | −0.01737 | 0.00336 | −0.08781 | 0.02616 | 0.01253 | −0.00741 | 0.04145 |
| Enteroendocrine.TFPI2.TPH1 | 0.11871 | 0.10678 | 0.08003 | 0.01010 | −0.10304 | 0.00745 | 0.04194 |
| cDC2.CLEC10A.FCGR2B | −0.01409 | 0.02009 | 0.12387 | 0.00682 | −0.02027 | −0.02607 | −0.26495 |
| Paneth.DEFA6.ITLN2 | 0.08078 | 0.04640 | 0.00344 | 0.01645 | −0.06711 | −0.01146 | 0.01017 |
| Myeloid.MKI67.IGKC | −0.01059 | −0.00697 | −0.16761 | 0.01405 | 0.05727 | 0.00241 | 0.03559 |
| EC.MTRNR2L1.MT.ND3 | −0.01075 | 0.04446 | 0.03010 | 0.01709 | 0.00013 | −0.00827 | 0.05653 |
| EC.PLCG2.MAFB | −0.01354 | 0.01813 | 0.11076 | 0.04018 | 0.03108 | −0.00628 | 0.02667 |
| T.GNLY.MT.CO2 | −0.08127 | 0.09649 | −0.04450 | −0.05179 | −0.02623 | −0.01544 | 0.00710 |
| T.MT.CO2.MT.CO1 | −0.09445 | −0.05803 | −0.07463 | 0.05525 | −0.04901 | 0.02267 | 0.04352 |
| Goblet.TFF1.TPSG1 | −0.00279 | 0.00023 | 0.08697 | −0.09142 | −0.01723 | 0.02884 | 0.02700 |
| EC.MIF.AC0904981 | 0.03788 | −0.05242 | 0.03510 | 0.03471 | −0.00907 | 0.05930 | 0.00652 |
| Goblet.FCGBP.SPINK4 | −0.02183 | −0.02224 | −0.22528 | −0.01216 | 0.04696 | 0.06296 | −0.00338 |
| Enteroendocrine.NEUROG3.MLN | −0.02316 | 0.07850 | 0.09386 | −0.00935 | −0.08102 | −0.04644 | 0.01620 |
| T.CTLA4.CD27 | −0.10883 | −0.06693 | 0.00828 | 0.08373 | −0.03020 | 0.00833 | 0.00243 |
| DC.CCR7.FSCN1 | −0.02825 | −0.01982 | −0.07259 | 0.02738 | −0.06363 | −0.11327 | 0.15234 |
| NK.MKI67.GZMA | −0.01253 | 0.03164 | 0.01008 | 0.07756 | 0.04351 | −0.02646 | −0.02971 |
| NK.CCL3.CD160 | −0.00856 | −0.00028 | −0.23394 | −0.01197 | 0.05993 | 0.05155 | 0.00630 |
| cDC2.RALA.CD1C | 0.03442 | −0.02375 | 0.10063 | −0.01446 | −0.00631 | −0.01372 | 0.56258 |
| cDC2.ZFANDZA.INHBA | 0.03442 | −0.02375 | 0.10063 | −0.01446 | −0.00631 | −0.01372 | −0.19992 |
| T.NR3C1.CCR6 | 0.03431 | −0.02310 | 0.10219 | −0.01412 | −0.00599 | −0.01443 | −0.00177 |
| T.GNLY.HOPX | 0.03288 | −0.02284 | 0.09470 | −0.01495 | −0.00569 | −0.01497 | −0.00692 |
| Mono.Mac.CCL4.DDX3Y | 0.03780 | −0.02354 | 0.10164 | −0.01433 | −0.00812 | −0.01338 | 0.00096 |
| Mono.FCN1.HSPA1A | 0.03288 | −0.02304 | 0.09228 | −0.01026 | −0.00548 | −0.01692 | −0.02382 |
| T.TIGIT.CTLA4 | 0.03261 | −0.02240 | 0.09900 | −0.00976 | −0.00552 | −0.01826 | −0.02310 |
| Mono.Mac.DC.CXCL10.IFIT2 | 0.03223 | −0.02423 | 0.07673 | −0.00938 | −0.00361 | −0.01591 | −0.02731 |
| TA.PLCG2.SOX4 | −0.07004 | 0.01053 | 0.09987 | 0.05888 | 0.10222 | 0.07632 | 0.01873 |
| DC.LTB.IL22RA2 | 0.07677 | 0.02913 | −0.02475 | −0.00977 | 0.00886 | −0.03597 | 0.04706 |
| T.MT.CO2.MT.CO3 | −0.06142 | 0.03697 | −0.14228 | −0.02985 | −0.01655 | 0.01618 | −0.05996 |
| T.B2M.MT.CO2 | −0.02964 | 0.01417 | 0.09129 | −0.05876 | 0.02640 | 0.05434 | 0.00472 |
| NK.GNLY.FCGR3A | 0.02622 | −0.03070 | 0.03802 | −0.00690 | −0.00482 | −0.01059 | 0.00411 |
| Prolif.EC.UBE2C.HMGB2 | −0.08592 | 0.06909 | −0.00609 | −0.14653 | −0.09262 | 0.06406 | −0.01864 |
| T.MT.CO2.PTGER4 | −0.04610 | −0.04901 | 0.06804 | 0.01248 | −0.07694 | −0.00509 | −0.02862 |
| Enteroendocrine.TPH1.CHGA | 0.00093 | 0.01799 | 0.01546 | 0.02420 | −0.04016 | 0.02629 | 0.00798 |
| cDC1.CLEC9A.IDO1 | −0.02773 | 0.01445 | −0.17977 | −0.03962 | 0.04963 | 0.05453 | −0.52619 |
| Myeloid.MKI67.PCNA | 0.13667 | 0.00243 | −0.04440 | −0.02722 | −0.02581 | −0.03502 | −0.02750 |
| Mac.JUN.EGR1 | 0.03758 | −0.02536 | 0.01960 | −0.00803 | −0.00122 | −0.00645 | −0.01613 |
| Mono.FCN1.EIF4A3 | 0.02976 | −0.02477 | −0.03320 | −0.00797 | 0.01570 | −0.00386 | 0.03813 |
| Goblet.REG4.SPINK4 | 0.04339 | −0.06444 | 0.02217 | −0.01473 | 0.01588 | 0.02318 | 0.03541 |
| cDC1.CLEC9A.XCR1 | 0.06296 | 0.00920 | −0.09713 | −0.01168 | −0.02294 | 0.01927 | −0.05201 |

TABLE 4
Markers for all cell subsets in FG atlas (ordered by adj p value for each subset)

| cluster | gene | avg_logFC | cluster | gene | avg_logFC | cluster | gene | avg_logFC |
|---|---|---|---|---|---|---|---|---|
| FG.B.IGHD.FCER2 | IGHD | 1.75 | FG.EC.GSTA2.AADAC | MT1E | 0.97 | FG.Fibro.CCL11.FABP5 | FHL2 | 1.21 |
| FG.B.IGHD.FCER2 | FCER2 | 1.49 | FG.EC.GSTA2.AADAC | ATF3 | 0.97 | FG.Fibro.CCL11.FABP5 | RBP5 | 1.20 |
| FG.B.IGHD.FCER2 | CD83 | 1.47 | FG.EC.GSTA2.AADAC | HBEGF | 0.97 | FG.Fibro.CCL11.FABP5 | OGN | 1.19 |
| FG.B.IGHD.FCER2 | CD79A | 1.43 | FG.EC.GSTA2.AADAC | EGR1 | 0.97 | FG.Fibro.CCL11.FABP5 | PROS1 | 1.17 |
| FG.B.IGHD.FCER2 | MS4A1 | 1.39 | FG.EC.GSTA2.AADAC | HSPA1A | 0.97 | FG.Fibro.CCL11.FABP5 | SEPP1 | 1.17 |
| FG.B.IGHD.FCER2 | HLA-DQB1 | 1.35 | FG.EC.GSTA2.AADAC | SLC25A3 | 0.97 | FG.Fibro.CCL11.FABP5 | EFEMP2 | 1.16 |
| FG.B.IGHD.FCER2 | TCL1A | 1.33 | FG.EC.GSTA2.AADAC | OTC | 0.97 | FG.Fibro.CCL11.FABP5 | NDN | 1.16 |
| FG.B.IGHD.FCER2 | CD74 | 1.33 | FG.EC.GSTA2.AADAC | PEPD | 0.96 | FG.Fibro.CCL11.FABP5 | DKK3 | 1.15 |
| FG.B.IGHD.FCER2 | HVCN1 | 1.29 | FG.EC.GSTA2.AADAC | S100A16 | 0.96 | FG.Fibro.CCL11.FABP5 | COL18A1 | 1.14 |
| FG.B.IGHD.FCER2 | VPREB3 | 1.29 | FG.EC.GSTA2.AADAC | LIPH | 0.96 | FG.Fibro.CCL11.FABP5 | LOXL1 | 1.12 |
| FG.B.IGHD.FCER2 | HLA-DRB1 | 1.24 | FG.EC.GSTA2.AADAC | MT1H | 0.96 | FG.Fibro.CCL11.FABP5 | GSTM3 | 1.12 |
| FG.B.IGHD.FCER2 | CD72 | 1.23 | FG.EC.GSTA2.AADAC | TMEM45B | 0.95 | FG.Fibro.CCL11.FABP5 | TDO2 | 1.11 |
| FG.B.IGHD.FCER2 | HLA-DRA | 1.18 | FG.EC.GSTA2.AADAC | COX6C | 0.95 | FG.Fibro.CCL11.FABP5 | EFEMP1 | 1.10 |
| FG.B.IGHD.FCER2 | MEF2C | 1.17 | FG.EC.GSTA2.AADAC | CYBRD1 | 0.95 | FG.Fibro.CCL11.FABP5 | ECM1 | 1.09 |
| FG.B.IGHD.FCER2 | CD37 | 1.13 | FG.EC.GSTA2.AADAC | REEP6 | 0.95 | FG.Fibro.CCL11.FABP5 | EHD2 | 1.09 |
| FG.B.IGHD.FCER2 | HLA-DQA1 | 1.13 | FG.EC.GSTA2.AADAC | ACADVL | 0.95 | FG.Fibro.CCL11.FABP5 | OLFML3 | 1.09 |
| FG.B.IGHD.FCER2 | HLA-DPB1 | 1.06 | FG.EC.GSTA2.AADAC | TM4SF4 | 0.94 | FG.Fibro.CCL11.FABP5 | STMN2 | 1.08 |
| FG.B.IGHD.FCER2 | PHACTR1 | 1.06 | FG.EC.GSTA2.AADAC | CHCHD10 | 0.94 | FG.Fibro.CCL11.FABP5 | TSPAN4 | 1.08 |
| FG.B.IGHD.FCER2 | CD52 | 1.06 | FG.EC.GSTA2.AADAC | MYO1A | 0.94 | FG.Fibro.CCL11.FABP5 | ZEB2 | 1.08 |
| FG.B.IGHD.FCER2 | GPR18 | 1.03 | FG.EC.GSTA2.AADAC | CREB3L3 | 0.93 | FG.Fibro.CCL11.FABP5 | TIMP2 | 1.08 |
| FG.B.IGHD.FCER2 | CD79B | 1.02 | FG.EC.GSTA2.AADAC | CISD1 | 0.93 | FG.Fibro.CCL11.FABP5 | RP11-14N7.2 | 1.07 |
| FG.B.IGHD.FCER2 | CD22 | 1.02 | FG.EC.GSTA2.AADAC | ZG16 | 0.93 | FG.Fibro.CCL11.FABP5 | MATN2 | 1.07 |
| FG.B.IGHD.FCER2 | HLA-DMB | 1.00 | FG.EC.GSTA2.AADAC | MT1F | 0.93 | FG.Fibro.CCL11.FABP5 | SERPINE2 | 1.07 |
| FG.B.IGHD.FCER2 | LY9 | 0.99 | FG.EC.GSTA2.AADAC | MT-ND1 | 0.93 | FG.Fibro.CCL11.FABP5 | IGFBP5 | 1.07 |
| FG.B.IGHD.FCER2 | SELL | 0.97 | FG.EC.GSTA2.AADAC | DECR1 | 0.93 | FG.Fibro.CCL11.FABP5 | CST3 | 1.07 |
| FG.B.IGHD.FCER2 | HLA-DPA1 | 0.97 | FG.EC.GSTA2.AADAC | ATP5D | 0.93 | FG.Fibro.CCL11.FABP5 | CNN3 | 1.05 |
| FG.B.IGHD.FCER2 | BTG1 | 0.97 | FG.EC.GSTA2.AADAC | NDUFA4 | 0.92 | FG.Fibro.CCL11.FABP5 | PPIC | 1.04 |
| FG.B.IGHD.FCER2 | CXCR4 | 0.96 | FG.EC.GSTA2.AADAC | MT-ND5 | 0.92 | FG.Fibro.CCL11.FABP5 | FNDC1 | 1.03 |
| FG.B.IGHD.FCER2 | BANK1 | 0.95 | FG.EC.GSTA2.AADAC | SLC25A37 | 0.92 | FG.Fibro.CCL11.FABP5 | SERPINH1 | 1.03 |
| FG.B.IGHD.FCER2 | CD19 | 0.91 | FG.EC.GSTA2.AADAC | PHLDA2 | 0.92 | FG.Fibro.CCL11.FABP5 | NNMT | 1.03 |
| FG.B.IGHD.FCER2 | RHOH | 0.91 | FG.EC.GSTA2.AADAC | MT-CO2 | 0.91 | FG.Fibro.CCL11.FABP5 | PLAT | 1.02 |
| FG.B.IGHD.FCER2 | LAPTM5 | 0.91 | FG.EC.GSTA2.AADAC | VNN1 | 0.90 | FG.Fibro.CCL11.FABP5 | NFIA | 1.02 |
| FG.B.IGHD.FCER2 | HLA-DMA | 0.90 | FG.EC.GSTA2.AADAC | DDC | 0.90 | FG.Fibro.CCL11.FABP5 | BCHE | 1.02 |
| FG.B.IGHD.FCER2 | BIRC3 | 0.90 | FG.EC.GSTA2.AADAC | MT-CYB | 0.90 | FG.Fibro.CCL11.FABP5 | COL12A1 | 1.02 |
| FG.B.IGHD.FCER2 | TNFRSF13C | 0.90 | FG.EC.GSTA2.AADAC | GSTO1 | 0.90 | FG.Fibro.CCL11.FABP5 | IGFBP4 | 1.02 |
| FG.B.IGHD.FCER2 | NCF1 | 0.84 | FG.EC.GSTA2.AADAC | VDAC1 | 0.90 | FG.Fibro.CCL11.FABP5 | FXYD6 | 1.01 |
| FG.B.IGHD.FCER2 | RPL18A | 0.77 | FG.EC.GSTA2.AADAC | FUOM | 0.89 | FG.Fibro.CCL11.FABP5 | ITIH5 | 1.00 |
| FG.B.IGHD.FCER2 | HERPUD1 | 0.77 | FG.EC.GSTA2.AADAC | ACE2 | 0.89 | FG.Fibro.CCL11.FABP5 | IL34 | 1.00 |
| FG.B.IGHD.FCER2 | SLC2A3 | 0.77 | FG.EC.GSTA2.AADAC | UQCRC2 | 0.88 | FG.Fibro.CCL11.FABP5 | C2 | 0.99 |
| FG.B.IGHD.FCER2 | CYTIP | 0.74 | FG.EC.GSTA2.AADAC | SLC22A18 | 0.88 | FG.Fibro.CCL11.FABP5 | C1orf21 | 0.98 |
| FG.B.IGHD.FCER2 | RPS5 | 0.64 | FG.EC.GSTA2.AADAC | MT-CO1 | 0.88 | FG.Fibro.CCL11.FABP5 | COL14A1 | 0.98 |
| FG.B.IGHD.FCER2 | JUNB | 0.59 | FG.EC.GSTA2.AADAC | COX8A | 0.88 | FG.Fibro.CCL11.FABP5 | SRPX | 0.97 |
| FG.B.IGHD.FCER2 | RPSA | 0.58 | FG.EC.GSTA2.AADAC | SAT2 | 0.88 | FG.Fibro.CCL11.FABP5 | PLEKHH2 | 0.97 |
| FG.B.IGHD.FCER2 | RPS10 | 0.54 | FG.EC.GSTA2.AADAC | ECHS1 | 0.88 | FG.Fibro.CCL11.FABP5 | FSTL1 | 0.96 |
| FG.B.IGHD.FCER2 | FAU | 0.53 | FG.EC.GSTA2.AADAC | ATP5J | 0.88 | FG.Fibro.CCL11.FABP5 | FOXF1 | 0.96 |
| FG.B.IGHD.FCER2 | RPL8 | 0.53 | FG.EC.GSTA2.AADAC | NDUFB9 | 0.87 | FG.Fibro.CCL11.FABP5 | SLC9A3R2 | 0.96 |
| FG.B.IGHD.FCER2 | RPS27 | 0.52 | FG.EC.GSTA2.AADAC | SLC4A7 | 0.87 | FG.Fibro.CCL11.FABP5 | RAB34 | 0.95 |
| FG.B.IGHD.FCER2 | RPS19 | 0.52 | FG.EC.GSTA2.AADAC | HSD17B11 | 0.87 | FG.Fibro.CCL11.FABP5 | FABP4 | 0.95 |
| FG.B.IGHD.FCER2 | RPS17 | 0.52 | FG.EC.GSTA2.AADAC | S100A6 | 0.86 | FG.Fibro.CCL11.FABP5 | COLEC11 | 0.94 |
| FG.B.IGHD.FCER2 | RPL26 | 0.51 | FG.EC.GSTA2.AADAC | SERPINB6 | 0.86 | FG.Fibro.CCL11.FABP5 | DDR2 | 0.94 |
| FG.B.IGHD.FCER2 | RPS23 | 0.51 | FG.EC.GSTA2.AADAC | CALM1 | 0.86 | FG.Fibro.CCL11.FABP5 | COL15A1 | 0.94 |
| FG.B.IGHD.FCER2 | RPL15 | 0.50 | FG.EC.GSTA2.AADAC | ETHE1 | 0.86 | FG.Fibro.CCL11.FABP5 | BMP4 | 0.93 |
| FG.B.IGHD.FCER2 | RPS11 | 0.49 | FG.EC.GSTA2.AADAC | FAM46A | 0.86 | FG.Fibro.CCL11.FABP5 | MXRA8 | 0.93 |
| FG.B.IGHD.FCER2 | RPL10A | 0.48 | FG.EC.GSTA2.AADAC | COX6B1 | 0.86 | FG.Fibro.CCL11.FABP5 | RARRES1 | 0.93 |
| FG.B.IGHD.FCER2 | RPL12 | 0.47 | FG.EC.GSTA2.AADAC | GNA11 | 0.86 | FG.Fibro.CCL11.FABP5 | FIBIN | 0.92 |
| FG.B.IGHD.FCER2 | RPL41 | 0.47 | FG.EC.GSTA2.AADAC | RBM47 | 0.86 | FG.Fibro.CCL11.FABP5 | LAMA4 | 0.91 |
| FG.B.IGHD.FCER2 | RPS8 | 0.46 | FG.EC.GSTA2.AADAC | VMP1 | 0.86 | FG.Fibro.CCL11.FABP5 | COL5A2 | 0.91 |
| FG.B.IGHD.FCER2 | RPL18 | 0.46 | FG.EC.GSTA2.AADAC | ARL4A | 0.86 | FG.Fibro.CCL11.FABP5 | MFGE8 | 0.90 |
| FG.B.IGHD.FCER2 | CD69 | 0.46 | FG.EC.GSTA2.AADAC | GLS | 0.85 | FG.Fibro.CCL11.FABP5 | FAM127A | 0.89 |
| FG.B.IGHD.FCER2 | RPL30 | 0.45 | FG.EC.GSTA2.AADAC | CHPT1 | 0.85 | FG.Fibro.CCL11.FABP5 | GSTM5 | 0.88 |
| FG.B.IGHD.FCER2 | RPL23 | 0.44 | FG.EC.GSTA2.AADAC | MT1X | 0.85 | FG.Fibro.CCL11.FABP5 | CPQ | 0.88 |
| FG.B.IGHD.FCER2 | RPS2 | 0.44 | FG.EC.GSTA2.AADAC | SLC35G1 | 0.85 | FG.Fibro.CCL11.FABP5 | COL5A1 | 0.88 |
| FG.B.IGHD.FCER2 | RPLP2 | 0.44 | FG.EC.GSTA2.AADAC | ATP5A1 | 0.85 | FG.Fibro.CCL11.FABP5 | NDUFA4L2 | 0.87 |
| FG.B.IGHD.FCER2 | RPL23A | 0.44 | FG.EC.GSTA2.AADAC | GNG12 | 0.85 | FG.Fibro.CCL11.FABP5 | CP | 0.87 |
| FG.B.IGHD.FCER2 | RPS27A | 0.44 | FG.EC.GSTA2.AADAC | GSTK1 | 0.84 | FG.Fibro.CCL11.FABP5 | TGFB111 | 0.84 |
| FG.B.IGHD.FCER2 | RPS21 | 0.44 | FG.EC.GSTA2.AADAC | SLC13A2 | 0.84 | FG.Fibro.CCL11.FABP5 | HTRA1 | 0.84 |
| FG.B.IGHD.FCER2 | RPL39 | 0.43 | FG.EC.GSTA2.AADAC | TPM1 | 0.84 | FG.Fibro.CCL11.FABP5 | GPC6 | 0.83 |
| FG.B.IGHD.FCER2 | RPL13 | 0.43 | FG.EC.GSTA2.AADAC | SLC51B | 0.84 | FG.Fibro.CCL11.FABP5 | RBPMS | 0.83 |
| FG.B.IGHD.FCER2 | RPL13A | 0.42 | FG.EC.GSTA2.AADAC | LGALS3BP | 0.84 | FG.Fibro.CCL11.FABP5 | PAM | 0.81 |
| FG.B.IGHD.FCER2 | GNB2L1 | 0.42 | FG.EC.GSTA2.AADAC | OCIAD2 | 0.83 | FG.Fibro.CCL11.FABP5 | CRISPLD2 | 0.80 |
| FG.B.IGHD.FCER2 | RPL19 | 0.42 | FG.EC.GSTA2.AADAC | GSTM4 | 0.83 | FG.Fibro.CCL11.FABP5 | SFRP1 | 0.80 |
| FG.B.IGHD.FCER2 | RPS9 | 0.41 | FG.EC.GSTA2.AADAC | SCP2 | 0.83 | FG.Fibro.CCL11.FABP5 | SCT | 0.80 |
| FG.B.IGHD.FCER2 | RPL11 | 0.41 | FG.EC.GSTA2.AADAC | ETFB | 0.83 | FG.Fibro.CCL11.FABP5 | TRIP6 | 0.79 |
| FG.B.IGHD.FCER2 | RPL9 | 0.41 | FG.EC.GSTA2.AADAC | GLRX | 0.83 | FG.Fibro.CCL11.FABP5 | TIMP3 | 0.78 |
| FG.B.IGHD.FCER2 | RPL35 | 0.40 | FG.EC.GSTA2.AADAC | FTL | 0.83 | FG.Fibro.CCL11.FABP5 | NEXN | 0.78 |
| FG.B.IGHD.FCER2 | RPL21 | 0.39 | FG.EC.GSTA2.AADAC | TXNDC17 | 0.83 | FG.Fibro.CCL11.FABP5 | NID1 | 0.78 |
| FG.B.IGHD.FCER2 | RPL29 | 0.39 | FG.EC.GSTA2.AADAC | MT-ND4 | 0.83 | FG.Fibro.CCL11.FABP5 | GULP1 | 0.78 |
| FG.B.IGHD.FCER2 | RPL32 | 0.39 | FG.EC.GSTA2.AADAC | SPINK1 | 0.83 | FG.Fibro.CCL11.FABP5 | CRIP2 | 0.78 |
| FG.B.IGHD.FCER2 | RPS3A | 0.38 | FG.EC.GSTA2.AADAC | FAM3C | 0.83 | FG.Fibro.CCL11.FABP5 | ALDH1A3 | 0.78 |
| FG.B.IGHD.FCER2 | RPL28 | 0.38 | FG.EC.GSTA2.AADAC | FLJ22763 | 0.82 | FG.Fibro.CCL11.FABP5 | FRZB | 0.74 |
| FG.B.IGHD.FCER2 | RPL31 | 0.37 | FG.EC.GSTA2.AADAC | AKR1A1 | 0.82 | FG.Fibro.CCL11.FABP5 | PCDH18 | 0.74 |
| FG.B.IGHD.FCER2 | RPS12 | 0.37 | FG.EC.GSTA2.AADAC | TPI1 | 0.82 | FG.Fibro.CCL11.FABP5 | AEBP1 | 0.73 |
| FG.B.IGHD.FCER2 | RPS7 | 0.37 | FG.EC.GSTA2.AADAC | ATP5H | 0.82 | FG.Fibro.CCL11.FABP5 | FAM198B | 0.73 |
| FG.B.IGHD.FCER2 | RPS25 | 0.37 | FG.EC.GSTA2.AADAC | SLC4A4 | 0.82 | FG.Fibro.CCL11.FABP5 | FBN1 | 0.73 |
| FG.B.IGHD.FCER2 | RPS13 | 0.37 | FG.EC.GSTA2.AADAC | EPHX2 | 0.82 | FG.Fibro.CCL11.FABP5 | MAMDC2 | 0.72 |
| FG.B.IGHD.FCER2 | RPL10 | 0.36 | FG.EC.GSTA2.AADAC | GDA | 0.81 | FG.Fibro.CCL11.FABP5 | BMP5 | 0.72 |
| FG.B.IGHD.FCER2 | RPS20 | 0.36 | FG.EC.GSTA2.AADAC | IQGAP2 | 0.81 | FG.Fibro.CCL11.FABP5 | HOXC9 | 0.71 |
| FG.B.IGHD.FCER2 | RPS15 | 0.35 | FG.EC.GSTA2.AADAC | NDUFB10 | 0.80 | FG.Fibro.CCL11.FABP5 | PDGFRA | 0.71 |
| FG.B.IGHD.FCER2 | RPL34 | 0.34 | FG.EC.GSTA2.AADAC | RAMP1 | 0.80 | FG.Fibro.CCL11.FABP5 | AKAP12 | 0.70 |
| FG.B.IGHD.FCER2 | RPL35A | 0.34 | FG.EC.GSTA2.AADAC | SLC39A4 | 0.80 | FG.Fibro.CCL11.FABP5 | WNT2B | 0.70 |
| FG.B.IGHD.FCER2 | RPL7 | 0.34 | FG.EC.GSTA2.AADAC | DNPH1 | 0.80 | FG.Fibro.CCL11.FABP5 | TCEAL7 | 0.70 |
| FG.B.IGHD.FCER2 | RPS16 | 0.34 | FG.EC.GSTA2.AADAC | ATP5F1 | 0.80 | FG.Fibro.CCL11.FABP5 | ISLR | 0.70 |
| FG.B.IGHD.FCER2 | RPL37 | 0.33 | FG.EC.GSTA2.AADAC | AOC1 | 0.80 | FG.Fibro.CCL11.FABP5 | ANGPTL2 | 0.69 |
| FG.B.IGHD.FCER2 | RPL27A | 0.30 | FG.EC.GSTA2.AADAC | NAPRT | 0.80 | FG.Fibro.CCL11.FABP5 | PDGFRB | 0.69 |
| FG.B.IGHD.FCER2 | RPS6 | 0.30 | FG.EC.GSTA2.AADAC | SPINT2 | 0.79 | FG.Fibro.CCL11.FABP5 | KCNS3 | 0.68 |
| FG.B.IGHD.FCER2 | RPS15A | 0.28 | FG.EC.GSTA2.AADAC | TST | 0.79 | FG.Fibro.CCL11.FABP5 | PCDH7 | 0.68 |
| FG.B.IGHD.FCER2 | RPL3 | 0.27 | FG.EC.GSTA2.AADAC | PYCARD | 0.79 | FG.Fibro.CCL11.FABP5 | C1QTNF2 | 0.68 |
| FG.B.IGHD.FCER2 | RP11-693J15.5 | 0.82 | FG.EC.GSTA2.AADAC | DHRS4L2 | 0.78 | FG.Fibro.CCL11.FABP5 | TNFAIP6 | 0.67 |
| FG.B.IGHD.FCER2 | CHI3L2 | 0.80 | FG.EC.GSTA2.AADAC | CLTB | 0.77 | FG.Fibro.CCL11.FABP5 | PAMR1 | 0.67 |
| FG.B.IGHD.FCER2 | H3F3A | 0.44 | FG.EC.GSTA2.AADAC | ALKBH7 | 0.77 | FG.Fibro.CCL11.FABP5 | TRIL | 0.67 |
| FG.B.IGHD.FCER2 | CYBA | 0.48 | FG.EC.GSTA2.AADAC | ADH4 | 0.77 | FG.Fibro.CCL11.FABP5 | OLFML1 | 0.67 |
| FG.B.IGLC6.IGLC7 | IGLC7 | 2.90 | FG.EC.GSTA2.AADAC | RTN4 | 0.77 | FG.Fibro.CCL11.FABP5 | ACVRL1 | 0.67 |
| FG.B.IGLC6.IGLC7 | IGLC6 | 1.92 | FG.EC.GSTA2.AADAC | S100A10 | 0.77 | FG.Fibro.CCL11.FABP5 | GLT8D2 | 0.65 |
| FG.B.TNFRSF13B.GPR183 | VPREB3 | 1.33 | FG.EC.GSTA2.AADAC | COA3 | 0.77 | FG.Fibro.CCL11.FABP5 | FKBP10 | 0.63 |
| FG.B.TNFRSF13B.GPR183 | BANK1 | 1.26 | FG.EC.GSTA2.AADAC | UQCRFS1 | 0.77 | FG.Fibro.CCL11.FABP5 | MXRA5 | 0.62 |
| FG.B.TNFRSF13B.GPR183 | MS4A1 | 1.09 | FG.EC.GSTA2.AADAC | NDUFV1 | 0.76 | FG.Fibro.CCL11.FABP5 | ELANE | 0.58 |
| FG.B.TNFRSF13B.GPR183 | CD79A | 0.99 | FG.EC.GSTA2.AADAC | PGRMC2 | 0.76 | FG.Fibro.CCL11.FABP5 | NOVA1 | 0.63 |
| FG.B.TNFRSF13B.GPR183 | RGS2 | 0.98 | FG.EC.GSTA2.AADAC | ETFA | 0.76 | FG.Fibro.CCL11.FABP5 | BST2 | 1.25 |
| FG.B.TNFRSF13B.GPR183 | RP5-887A10.1 | 0.98 | FG.EC.GSTA2.AADAC | ONECUT2 | 0.76 | FG.Fibro.CCL11.FABP5 | RP11-834C11.4 | 0.54 |
| FG.B.TNFRSF13B.GPR183 | TNFRSF13B | 0.97 | FG.EC.GSTA2.AADAC | USMG5 | 0.76 | FG.Fibro.CCL11.FABP5 | MMP23B | 0.62 |
| FG.B.TNFRSF13B.GPR183 | ARHGAP24 | 0.92 | FG.EC.GSTA2.AADAC | CIDEB | 0.76 | FG.Fibro.CCL11.FABP5 | TMEM100 | 0.61 |
| FG.B.TNFRSF13B.GPR183 | GPR183 | 0.91 | FG.EC.GSTA2.AADAC | SLC7A9 | 0.76 | FG.Fibro.CCL11.FABP5 | PDLIM3 | 0.73 |
| FG.B.TNFRSF13B.GPR183 | CD83 | 0.89 | FG.EC.GSTA2.AADAC | SDHB | 0.76 | FG.Fibro.CCL11.FABP5 | TCEAL4 | 1.02 |
| FG.B.TNFRSF13B.GPR183 | CD24 | 0.89 | FG.EC.GSTA2.AADAC | NCOA4 | 0.75 | FG.Fibro.CCL11.FABP5 | EVA1A | 0.56 |
| FG.B.TNFRSF13B.GPR183 | LY86 | 0.86 | FG.EC.GSTA2.AADAC | SELENBP1 | 0.75 | FG.Fibro.CCL11.FABP5 | SHISA3 | 0.54 |
| FG.B.TNFRSF13B.GPR183 | TNFRSF13C | 0.86 | FG.EC.GSTA2.AADAC | ALDH2 | 0.75 | FG.Fibro.CCL13.CFD | CCL13 | 4.43 |
| FG.B.TNFRSF13B.GPR183 | CD37 | 0.83 | FG.EC.GSTA2.AADAC | TMEM256 | 0.75 | FG.Fibro.CCL13.CFD | ADAMDEC1 | 3.59 |
| FG.B.TNFRSF13B.GPR183 | SMIM14 | 0.83 | FG.EC.GSTA2.AADAC | UQCR10 | 0.75 | FG.Fibro.CCL13.CFD | CFD | 3.46 |
| FG.B.TNFRSF13B.GPR183 | CD74 | 0.81 | FG.EC.GSTA2.AADAC | MEP1A | 0.75 | FG.Fibro.CCL13.CFD | LUM | 3.45 |
| FG.B.TNFRSF13B.GPR183 | CXCR4 | 0.77 | FG.EC.GSTA2.AADAC | EFNA1 | 0.75 | FG.Fibro.CCL13.CFD | CCL11 | 3.24 |
| FG.B.TNFRSF13B.GPR183 | HLA-DQB1 | 0.76 | FG.EC.GSTA2.AADAC | IDH1 | 0.75 | FG.Fibro.CCL13.CFD | CXCL14 | 3.19 |
| FG.B.TNFRSF13B.GPR183 | LTB | 0.72 | FG.EC.GSTA2.AADAC | APOA4 | 0.75 | FG.Fibro.CCL13.CFD | DCN | 3.06 |
| FG.B.TNFRSF13B.GPR183 | HLA-DRA | 0.66 | FG.EC.GSTA2.AADAC | B3GNT5 | 0.74 | FG.Fibro.CCL13.CFD | MFAP4 | 2.98 |
| FG.B.TNFRSF13B.GPR183 | RPL23A | 0.59 | FG.EC.GSTA2.AADAC | BLVRB | 0.74 | FG.Fibro.CCL13.CFD | APOE | 2.96 |
| FG.B.TNFRSF13B.GPR183 | RPLP2 | 0.59 | FG.EC.GSTA2.AADAC | LDHA | 0.74 | FG.Fibro.CCL13.CFD | CFH | 2.68 |
| FG.B.TNFRSF13B.GPR183 | HLA-DQA1 | 0.58 | FG.EC.GSTA2.AADAC | MTCH2 | 0.74 | FG.Fibro.CCL13.CFD | CCL8 | 2.67 |
| FG.B.TNFRSF13B.GPR183 | CD79B | 0.58 | FG.EC.GSTA2.AADAC | MT-ND3 | 0.74 | FG.Fibro.CCL13.CFD | C1S | 2.62 |
| FG.B.TNFRSF13B.GPR183 | RPS8 | 0.54 | FG.EC.GSTA2.AADAC | MT-ND2 | 0.74 | FG.Fibro.CCL13.CFD | RARRES2 | 2.58 |
| FG.B.TNFRSF13B.GPR183 | HLA-DPB1 | 0.53 | FG.EC.GSTA2.AADAC | GOLIM4 | 0.73 | FG.Fibro.CCL13.CFD | C1R | 2.48 |
| FG.B.TNFRSF13B.GPR183 | CD52 | 0.52 | FG.EC.GSTA2.AADAC | SFN | 0.73 | FG.Fibro.CCL13.CFD | CCL2 | 2.46 |
| FG.B.TNFRSF13B.GPR183 | RPL27A | 0.51 | FG.EC.GSTA2.AADAC | ACOX1 | 0.73 | FG.Fibro.CCL13.CFD | CALD1 | 2.43 |
| FG.B.TNFRSF13B.GPR183 | RPS29 | 0.51 | FG.EC.GSTA2.AADAC | SUCLG2 | 0.73 | FG.Fibro.CCL13.CFD | FBLN1 | 2.37 |
| FG.B.TNFRSF13B.GPR183 | RPL11 | 0.51 | FG.EC.GSTA2.AADAC | FAM195A | 0.73 | FG.Fibro.CCL13.CFD | IGFBP7 | 2.37 |
| FG.B.TNFRSF13B.GPR183 | RPS25 | 0.50 | FG.EC.GSTA2.AADAC | SLC3A1 | 0.73 | FG.Fibro.CCL13.CFD | GPX3 | 2.35 |
| FG.B.TNFRSF13B.GPR183 | RPS27 | 0.50 | FG.EC.GSTA2.AADAC | UQCRH | 0.73 | FG.Fibro.CCL13.CFD | IFITM3 | 2.34 |
| FG.B.TNFRSF13B.GPR183 | RPS21 | 0.49 | FG.EC.GSTA2.AADAC | TMEM41A | 0.73 | FG.Fibro.CCL13.CFD | ABCA8 | 2.30 |
| FG.B.TNFRSF13B.GPR183 | RPS11 | 0.49 | FG.EC.GSTA2.AADAC | ATP5I | 0.73 | FG.Fibro.CCL13.CFD | COL3A1 | 2.22 |
| FG.B.TNFRSF13B.GPR183 | RPS23 | 0.49 | FG.EC.GSTA2.AADAC | EDF1 | 0.73 | FG.Fibro.CCL13.CFD | COL1A2 | 2.15 |
| FG.B.TNFRSF13B.GPR183 | RPL39 | 0.48 | FG.EC.GSTA2.AADAC | HSPE1 | 0.73 | FG.Fibro.CCL13.CFD | PPP1R14A | 2.15 |
| FG.B.TNFRSF13B.GPR183 | RPSA | 0.48 | FG.EC.GSTA2.AADAC | STAP2 | 0.72 | FG.Fibro.CCL13.CFD | TMEM176B | 2.14 |
| FG.B.TNFRSF13B.GPR183 | RPL41 | 0.47 | FG.EC.GSTA2.AADAC | SDHA | 0.72 | FG.Fibro.CCL13.CFD | A2M | 2.12 |
| FG.B.TNFRSF13B.GPR183 | RPL30 | 0.47 | FG.EC.GSTA2.AADAC | GIPC2 | 0.72 | FG.Fibro.CCL13.CFD | RBP1 | 2.03 |
| FG.B.TNFRSF13B.GPR183 | RPS20 | 0.47 | FG.EC.GSTA2.AADAC | PRR15L | 0.72 | FG.Fibro.CCL13.CFD | CTSC | 1.99 |
| FG.B.TNFRSF13B.GPR183 | RPS5 | 0.46 | FG.EC.GSTA2.AADAC | PLS1 | 0.72 | FG.Fibro.CCL13.CFD | PLTP | 1.98 |
| FG.B.TNFRSF13B.GPR183 | RPS14 | 0.46 | FG.EC.GSTA2.AADAC | LCT | 0.72 | FG.Fibro.CCL13.CFD | CXCL12 | 1.97 |
| FG.B.TNFRSF13B.GPR183 | RPL13A | 0.45 | FG.EC.GSTA2.AADAC | CYP2C18 | 0.72 | FG.Fibro.CCL13.CFD | PLPP1 | 1.95 |
| FG.B.TNFRSF13B.GPR183 | RPS4X | 0.45 | FG.EC.GSTA2.AADAC | PPP1R14D | 0.72 | FG.Fibro.CCL13.CFD | HAPLN1 | 1.95 |
| FG.B.TNFRSF13B.GPR183 | RPS28 | 0.45 | FG.EC.GSTA2.AADAC | SLC37A4 | 0.72 | FG.Fibro.CCL13.CFD | ADH1B | 1.94 |
| FG.B.TNFRSF13B.GPR183 | RPL19 | 0.45 | FG.EC.GSTA2.AADAC | CD2AP | 0.71 | FG.Fibro.CCL13.CFD | LGALS1 | 1.93 |
| FG.B.TNFRSF13B.GPR183 | RPL9 | 0.44 | FG.EC.GSTA2.AADAC | ADI1 | 0.71 | FG.Fibro.CCL13.CFD | EDIL3 | 1.91 |
| FG.B.TNFRSF13B.GPR183 | RPL31 | 0.44 | FG.EC.GSTA2.AADAC | SLC6A19 | 0.71 | FG.Fibro.CCL13.CFD | QSOX1 | 1.90 |
| FG.B.TNFRSF13B.GPR183 | RPL27 | 0.44 | FG.EC.GSTA2.AADAC | AKR7A2 | 0.71 | FG.Fibro.CCL13.CFD | PTGDS | 1.89 |
| FG.B.TNFRSF13B.GPR183 | BTG1 | 0.44 | FG.EC.GSTA2.AADAC | HSPD1 | 0.70 | FG.Fibro.CCL13.CFD | SPARCL1 | 1.88 |
| FG.B.TNFRSF13B.GPR183 | RPS2 | 0.44 | FG.EC.GSTA2.AADAC | EIF6 | 0.70 | FG.Fibro.CCL13.CFD | TFPI | 1.84 |
| FG.B.TNFRSF13B.GPR183 | RPS16 | 0.43 | FG.EC.GSTA2.AADAC | PDK4 | 0.70 | FG.Fibro.CCL13.CFD | SERPING1 | 1.84 |
| FG.B.TNFRSF13B.GPR183 | FAU | 0.43 | FG.EC.GSTA2.AADAC | NDUFA1 | 0.70 | FG.Fibro.CCL13.CFD | GGT5 | 1.83 |
| FG.B.TNFRSF13B.GPR183 | RPS18 | 0.43 | FG.EC.GSTA2.AADAC | GCNT1 | 0.70 | FG.Fibro.CCL13.CFD | ASPN | 1.82 |
| FG.B.TNFRSF13B.GPR183 | RPL18A | 0.42 | FG.EC.GSTA2.AADAC | EDN1 | 0.70 | FG.Fibro.CCL13.CFD | CXCL1 | 1.80 |
| FG.B.TNFRSF13B.GPR183 | RPL26 | 0.42 | FG.EC.GSTA2.AADAC | SLC9A3R1 | 0.70 | FG.Fibro.CCL13.CFD | MEG3 | 1.76 |
| FG.B.TNFRSF13B.GPR183 | RPL38 | 0.42 | FG.EC.GSTA2.AADAC | GPD1 | 0.70 | FG.Fibro.CCL13.CFD | LTBP4 | 1.75 |
| FG.B.TNFRSF13B.GPR183 | HLA-DRB1 | 0.41 | FG.EC.GSTA2.AADAC | NDRG1 | 0.70 | FG.Fibro.CCL13.CFD | PLAC9 | 1.75 |
| FG.B.TNFRSF13B.GPR183 | HLA-DPA1 | 0.40 | FG.EC.GSTA2.AADAC | CYP27A1 | 0.70 | FG.Fibro.CCL13.CFD | TM4SF1 | 1.73 |
| FG.B.TNFRSF13B.GPR183 | RPL18 | 0.40 | FG.EC.GSTA2.AADAC | ATPIF1 | 0.69 | FG.Fibro.CCL13.CFD | NGFRAP1 | 1.72 |
| FG.B.TNFRSF13B.GPR183 | RPL32 | 0.39 | FG.EC.GSTA2.AADAC | MYL12B | 0.69 | FG.Fibro.CCL13.CFD | MMP2 | 1.72 |
| FG.B.TNFRSF13B.GPR183 | RPL14 | 0.39 | FG.EC.GSTA2.AADAC | CDH1 | 0.69 | FG.Fibro.CCL13.CFD | PTN | 1.70 |
| FG.B.TNFRSF13B.GPR183 | RPL8 | 0.39 | FG.EC.GSTA2.AADAC | FAM213A | 0.69 | FG.Fibro.CCL13.CFD | PLPP3 | 1.70 |
| FG.B.TNFRSF13B.GPR183 | RPL36 | 0.38 | FG.EC.GSTA2.AADAC | MYH14 | 0.69 | FG.Fibro.CCL13.CFD | FBLN5 | 1.69 |
| FG.B.TNFRSF13B.GPR183 | RPS3 | 0.38 | FG.EC.GSTA2.AADAC | RP3-335E1.1 | 0.68 | FG.Fibro.CCL13.CFD | GSN | 1.69 |
| FG.B.TNFRSF13B.GPR183 | RPL34 | 0.38 | FG.EC.GSTA2.AADAC | TSPO | 0.68 | FG.Fibro.CCL13.CFD | EMILIN1 | 1.68 |
| FG.B.TNFRSF13B.GPR183 | RPL10A | 0.38 | FG.EC.GSTA2.AADAC | MVP | 0.68 | FG.Fibro.CCL13.CFD | ANGPTL1 | 1.67 |
| FG.B.TNFRSF13B.GPR183 | RPS15A | 0.37 | FG.EC.GSTA2.AADAC | MGAT4B | 0.68 | FG.Fibro.CCL13.CFD | PMP22 | 1.66 |
| FG.B.TNFRSF13B.GPR183 | EEF1A1 | 0.37 | FG.EC.GSTA2.AADAC | DNAJC15 | 0.68 | FG.Fibro.CCL13.CFD | NUPR1 | 1.64 |
| FG.B.TNFRSF13B.GPR183 | RPL3 | 0.37 | FG.EC.GSTA2.AADAC | AHCYL1 | 0.68 | FG.Fibro.CCL13.CFD | TCF21 | 1.63 |
| FG.B.TNFRSF13B.GPR183 | RPS12 | 0.36 | FG.EC.GSTA2.AADAC | ACADS | 0.68 | FG.Fibro.CCL13.CFD | COL6A2 | 1.61 |
| FG.B.TNFRSF13B.GPR183 | RPS6 | 0.36 | FG.EC.GSTA2.AADAC | PRR13 | 0.68 | FG.Fibro.CCL13.CFD | LGALS3BP | 1.61 |
| FG.B.TNFRSF13B.GPR183 | RPL37 | 0.36 | FG.EC.GSTA2.AADAC | EPHX1 | 0.68 | FG.Fibro.CCL13.CFD | SPARC | 1.60 |
| FG.B.TNFRSF13B.GPR183 | RPS27A | 0.35 | FG.EC.GSTA2.AADAC | HSPA5 | 0.68 | FG.Fibro.CCL13.CFD | VCAM1 | 1.59 |
| FG.B.TNFRSF13B.GPR183 | RPS7 | 0.35 | FG.EC.GSTA2.AADAC | NDUFS3 | 0.67 | FG.Fibro.CCL13.CFD | CYGB | 1.59 |
| FG.B.TNFRSF13B.GPR183 | RPL35A | 0.33 | FG.EC.GSTA2.AADAC | PRSS8 | 0.67 | FG.Fibro.CCL13.CFD | IFI27 | 1.59 |
| FG.B.TNFRSF13B.GPR183 | RPL13 | 0.33 | FG.EC.GSTA2.AADAC | PLIN2 | 0.67 | FG.Fibro.CCL13.CFD | TMEM176A | 1.58 |
| FG.B.TNFRSF13B.GPR183 | RPL35 | 0.32 | FG.EC.GSTA2.AADAC | NDUFAB1 | 0.67 | FG.Fibro.CCL13.CFD | NBL1 | 1.54 |
| FG.B.TNFRSF13B.GPR183 | RPL12 | 0.32 | FG.EC.GSTA2.AADAC | NDUFS7 | 0.67 | FG.Fibro.CCL13.CFD | SPON2 | 1.53 |
| FG.B.TNFRSF13B.GPR183 | RPS3A | 0.32 | FG.EC.GSTA2.AADAC | SULT1A2 | 0.67 | FG.Fibro.CCL13.CFD | SERPINF1 | 1.51 |
| FG.B.TNFRSF13B.GPR183 | RPS13 | 0.32 | FG.EC.GSTA2.AADAC | ACAA1 | 0.67 | FG.Fibro.CCL13.CFD | COL1A1 | 1.50 |
| FG.B.TNFRSF13B.GPR183 | RPL21 | 0.31 | FG.EC.GSTA2.AADAC | CAT | 0.67 | FG.Fibro.CCL13.CFD | IGFBP6 | 1.50 |
| FG.B.TNFRSF13B.GPR183 | RPS15 | 0.28 | FG.EC.GSTA2.AADAC | ACOT7 | 0.66 | FG.Fibro.CCL13.CFD | MYL9 | 1.49 |
| FG.B.TNFRSF13B.GPR183 | RPL10 | 0.26 | FG.EC.GSTA2.AADAC | TSC22D1 | 0.66 | FG.Fibro.CCL13.CFD | FHL1 | 1.49 |
| FG.B.TNFRSF13B.GPR183 | FCRLA | 0.70 | FG.EC.GSTA2.AADAC | TM6SF2 | 0.66 | FG.Fibro.CCL13.CFD | CXCL6 | 1.48 |
| FG.B.TNFRSF13B.GPR183 | RPL23 | 0.43 | FG.EC.GSTA2.AADAC | CDX1 | 0.66 | FG.Fibro.CCL13.CFD | VCAN | 1.47 |
| FG.B/DZ.AICDA.IGKC | TCL1A | 2.06 | FG.EC.GSTA2.AADAC | KLF3 | 0.66 | FG.Fibro.CCL13.CFD | SNAI2 | 1.46 |
| FG.B/DZ.AICDA.IGKC | CD79B | 2.06 | FG.EC.GSTA2.AADAC | MT-ATP6 | 0.66 | FG.Fibro.CCL13.CFD | IL6ST | 1.46 |
| FG.B/DZ.AICDA.IGKC | SMIM14 | 1.70 | FG.EC.GSTA2.AADAC | LMO7 | 0.66 | FG.Fibro.CCL13.CFD | CLEC14A | 1.45 |
| FG.B/DZ.AICDA.IGKC | RGS13 | 1.68 | FG.EC.GSTA2.AADAC | CYCS | 0.65 | FG.Fibro.CCL13.CFD | S100A13 | 1.45 |
| FG.B/DZ.AICDA.IGKC | MARCKSL1 | 1.52 | FG.EC.GSTA2.AADAC | CAMTA1 | 0.65 | FG.Fibro.CCL13.CFD | C7 | 1.42 |
| FG.B/DZ.AICDA.IGKC | LIMD2 | 1.47 | FG.EC.GSTA2.AADAC | CLRN3 | 0.65 | FG.Fibro.CCL13.CFD | CLEC11A | 1.40 |
| FG.B/DZ.AICDA.IGKC | ISG20 | 1.47 | FG.EC.GSTA2.AADAC | USH1C | 0.65 | FG.Fibro.CCL13.CFD | SFTA1P | 1.39 |
| FG.B/DZ.AICDA.IGKC | NEIL1 | 1.37 | FG.EC.GSTA2.AADAC | TM9SF3 | 0.65 | FG.Fibro.CCL13.CFD | FILIP1L | 1.39 |
| FG.B/DZ.AICDA.IGKC | VPREB3 | 1.37 | FG.EC.GSTA2.AADAC | ERBB3 | 0.65 | FG.Fibro.CCL13.CFD | PCOLCE | 1.36 |
| FG.B/DZ.AICDA.IGKC | IRF8 | 1.36 | FG.EC.GSTA2.AADAC | AK2 | 0.64 | FG.Fibro.CCL13.CFD | PROCR | 1.34 |
| FG.B/DZ.AICDA.IGKC | SUGCT | 1.35 | FG.EC.GSTA2.AADAC | DSG2 | 0.64 | FG.Fibro.CCL13.CFD | FHL2 | 1.33 |
| FG.B/DZ.AICDA.IGKC | LTB | 1.34 | FG.EC.GSTA2.AADAC | SLC2A5 | 0.64 | FG.Fibro.CCL13.CFD | SEPP1 | 1.31 |
| FG.B/DZ.AICDA.IGKC | BCAS4 | 1.32 | FG.EC.GSTA2.AADAC | BCL2L15 | 0.64 | FG.Fibro.CCL13.CFD | SGCE | 1.30 |
| FG.B/DZ.AICDA.IGKC | RP11-231C14.7 | 1.32 | FG.EC.GSTA2.AADAC | ACAT1 | 0.64 | FG.Fibro.CCL13.CFD | LINC01082 | 1.26 |
| FG.B/DZ.AICDA.IGKC | AICDA | 1.31 | FG.EC.GSTA2.AADAC | CANX | 0.64 | FG.Fibro.CCL13.CFD | FXYD1 | 1.25 |
| FG.B/DZ.AICDA.IGKC | POU2AF1 | 1.29 | FG.EC.GSTA2.AADAC | TMC4 | 0.64 | FG.Fibro.CCL13.CFD | TNXB | 1.21 |
| FG.B/DZ.AICDA.IGKC | ATP5L | 1.26 | FG.EC.GSTA2.AADAC | ECH1 | 0.63 | FG.Fibro.CCL13.CFD | EFEMP1 | 1.21 |
| FG.B/DZ.AICDA.IGKC | ACTG1 | 1.22 | FG.EC.GSTA2.AADAC | GALM | 0.63 | FG.Fibro.CCL13.CFD | PRKCDBP | 1.20 |
| FG.B/DZ.AICDA.IGKC | METAP2 | 1.20 | FG.EC.GSTA2.AADAC | SERPINB1 | 0.63 | FG.Fibro.CCL13.CFD | BMP5 | 1.20 |
| FG.B/DZ.AICDA.IGKC | TCEA1 | 1.20 | FG.EC.GSTA2.AADAC | SAR1B | 0.63 | FG.Fibro.CCL13.CFD | PLEKHH2 | 1.19 |
| FG.B/DZ.AICDA.IGKC | BASP1 | 1.19 | FG.EC.GSTA2.AADAC | ADD3 | 0.63 | FG.Fibro.CCL13.CFD | EFEMP2 | 1.17 |
| FG.B/DZ.AICDA.IGKC | CD37 | 1.19 | FG.EC.GSTA2.AADAC | TDP2 | 0.63 | FG.Fibro.CCL13.CFD | ABCA6 | 1.17 |
| FG.B/DZ.AICDA.IGKC | SERPINA9 | 1.18 | FG.EC.GSTA2.AADAC | CDS1 | 0.63 | FG.Fibro.CCL13.CFD | CTSK | 1.17 |
| FG.B/DZ.AICDA.IGKC | NCF1 | 1.17 | FG.EC.GSTA2.AADAC | CDC42BPA | 0.63 | FG.Fibro.CCL13.CFD | DDR2 | 1.16 |
| FG.B/DZ.AICDA.IGKC | RGS16 | 1.17 | FG.EC.GSTA2.AADAC | RAB25 | 0.62 | FG.Fibro.CCL13.CFD | TPM2 | 1.16 |
| FG.B/DZ.AICDA.IGKC | SERF2 | 1.17 | FG.EC.GSTA2.AADAC | SLC39A14 | 0.62 | FG.Fibro.CCL13.CFD | PROS1 | 1.14 |
| FG.B/DZ.AICDA.IGKC | LRMP | 1.16 | FG.EC.GSTA2.AADAC | TTC36 | 0.62 | FG.Fibro.CCL13.CFD | NKX2-3 | 1.10 |
| FG.B/DZ.AICDA.IGKC | BIK | 1.15 | FG.EC.GSTA2.AADAC | NDUFS6 | 0.62 | FG.Fibro.CCL13.CFD | FN1 | 1.10 |
| FG.B/DZ.AICDA.IGKC | HMCES | 1.14 | FG.EC.GSTA2.AADAC | PERP | 0.62 | FG.Fibro.CCL13.CFD | TPPP3 | 1.09 |
| FG.B/DZ.AICDA.IGKC | UCP2 | 1.13 | FG.EC.GSTA2.AADAC | GK | 0.62 | FG.Fibro.CCL13.CFD | DKK3 | 1.08 |
| FG.B/DZ.AICDA.IGKC | SMARCB1 | 1.12 | FG.EC.GSTA2.AADAC | ABCG2 | 0.62 | FG.Fibro.CCL13.CFD | FSTL1 | 1.08 |
| FG.B/DZ.AICDA.IGKC | BCL7A | 1.12 | FG.EC.GSTA2.AADAC | DEGS2 | 0.62 | FG.Fibro.CCL13.CFD | PLAT | 1.07 |
| FG.B/DZ.AICDA.IGKC | CD79A | 1.11 | FG.EC.GSTA2.AADAC | PXMP2 | 0.62 | FG.Fibro.CCL13.CFD | GNG11 | 1.06 |
| FG.B/DZ.AICDA.IGKC | SUSD3 | 1.10 | FG.EC.GSTA2.AADAC | KRTCAP3 | 0.61 | FG.Fibro.CCL13.CFD | SDC2 | 1.05 |
| FG.B/DZ.AICDA.IGKC | GAPDH | 1.07 | FG.EC.GSTA2.AADAC | RETSAT | 0.61 | FG.Fibro.CCL13.CFD | PCDH7 | 1.05 |
| FG.B/DZ.AICDA.IGKC | UBE2J1 | 1.06 | FG.EC.GSTA2.AADAC | DSC2 | 0.61 | FG.Fibro.CCL13.CFD | SOD3 | 1.05 |
| FG.B/DZ.AICDA.IGKC | OAZ1 | 1.06 | FG.EC.GSTA2.AADAC | CLDN23 | 0.61 | FG.Fibro.CCL13.CFD | DPT | 1.04 |
| FG.B/DZ.AICDA.IGKC | ARPC3 | 1.06 | FG.EC.GSTA2.AADAC | NDUFC2 | 0.61 | FG.Fibro.CCL13.CFD | LAMA4 | 1.04 |
| FG.B/DZ.AICDA.IGKC | LAPTM5 | 1.04 | FG.EC.GSTA2.AADAC | SERINC2 | 0.60 | FG.Fibro.CCL13.CFD | MXRA8 | 1.03 |
| FG.B/DZ.AICDA.IGKC | HMGA1 | 1.03 | FG.EC.GSTA2.AADAC | S100G | 0.60 | FG.Fibro.CCL13.CFD | AKAP12 | 1.02 |
| FG.B/DZ.AICDA.IGKC | LSM10 | 1.03 | FG.EC.GSTA2.AADAC | HDHD3 | 0.60 | FG.Fibro.CCL13.CFD | NID1 | 1.02 |
| FG.B/DZ.AICDA.IGKC | RFTN1 | 1.03 | FG.EC.GSTA2.AADAC | FIS1 | 0.60 | FG.Fibro.CCL13.CFD | EHD2 | 1.02 |
| FG.B/DZ.AICDA.IGKC | GGA2 | 1.02 | FG.EC.GSTA2.AADAC | C11orf86 | 0.60 | FG.Fibro.CCL13.CFD | IL34 | 1.01 |
| FG.B/DZ.AICDA.IGKC | SPIB | 1.01 | FG.EC.GSTA2.AADAC | RAB11FIP1 | 0.60 | FG.Fibro.CCL13.CFD | BCHE | 1.01 |
| FG.B/DZ.AICDA.IGKC | AC079767.4 | 1.01 | FG.EC.GSTA2.AADAC | ATP1B3 | 0.60 | FG.Fibro.CCL13.CFD | ECM1 | 1.01 |
| FG.B/DZ.AICDA.IGKC | P2RX5 | 0.98 | FG.EC.GSTA2.AADAC | GPR160 | 0.60 | FG.Fibro.CCL13.CFD | SLC9A3R2 | 0.98 |
| FG.B/DZ.AICDA.IGKC | GCSAM | 0.97 | FG.EC.GSTA2.AADAC | CNDP2 | 0.60 | FG.Fibro.CCL13.CFD | COL5A2 | 0.96 |
| FG.B/DZ.AICDA.IGKC | MS4A1 | 0.97 | FG.EC.GSTA2.AADAC | MRPL12 | 0.59 | FG.Fibro.CCL13.CFD | COL15A1 | 0.96 |
| FG.B/DZ.AICDA.IGKC | PARP1 | 0.97 | FG.EC.GSTA2.AADAC | NDUFS2 | 0.59 | FG.Fibro.CCL13.CFD | LOXL1 | 0.95 |
| FG.B/DZ.AICDA.IGKC | MBD4 | 0.96 | FG.EC.GSTA2.AADAC | TFG | 0.59 | FG.Fibro.CCL13.CFD | COLEC11 | 0.94 |
| FG.B/DZ.AICDA.IGKC | TNFRSF13C | 0.95 | FG.EC.GSTA2.AADAC | CAMK2N1 | 0.59 | FG.Fibro.CCL13.CFD | KCNS3 | 0.92 |
| FG.B/DZ.AICDA.IGKC | DAAM1 | 0.95 | FG.EC.GSTA2.AADAC | ETS2 | 0.59 | FG.Fibro.CCL13.CFD | SFRP1 | 0.91 |
| FG.B/DZ.AICDA.IGKC | AC023590.1 | 0.94 | FG.EC.GSTA2.AADAC | NR1H4 | 0.59 | FG.Fibro.CCL13.CFD | OLFML3 | 0.88 |
| FG.B/DZ.AICDA.IGKC | RNGTT | 0.94 | FG.EC.GSTA2.AADAC | NDUFA6 | 0.59 | FG.Fibro.CCL13.CFD | FBN1 | 0.86 |
| FG.B/DZ.AICDA.IGKC | FCRLA | 0.92 | FG.EC.GSTA2.AADAC | UGT2B7 | 0.59 | FG.Fibro.CCL13.CFD | DIO2 | 0.86 |
| FG.B/DZ.AICDA.IGKC | CD22 | 0.91 | FG.EC.GSTA2.AADAC | MUC3A | 0.58 | FG.Fibro.CCL13.CFD | FOXF1 | 0.85 |
| FG.B/DZ.AICDA.IGKC | S1PR2 | 0.89 | FG.EC.GSTA2.AADAC | HNF4G | 0.58 | FG.Fibro.CCL13.CFD | PCDH18 | 0.81 |
| FG.B/DZ.AICDA.IGKC | DCAF12 | 0.86 | FG.EC.GSTA2.AADAC | LDLR | 0.58 | FG.Fibro.CCL13.CFD | MAMDC2 | 0.79 |
| FG.B/DZ.AICDA.IGKC | SH3TC1 | 0.85 | FG.EC.GSTA2.AADAC | ESRRA | 0.58 | FG.Fibro.CCL13.CFD | ITIH5 | 0.97 |
| FG.B/DZ.AICDA.IGKC | WDR66 | 0.83 | FG.EC.GSTA2.AADAC | LINC01133 | 0.58 | FG.Fibro.CCL13.CFD | COL6A3 | 0.99 |
| FG.B/DZ.AICDA.IGKC | ACTB | 0.76 | FG.EC.GSTA2.AADAC | NR0B2 | 0.58 | FG.Fibro.CCL13.CFD | THY1 | 1.00 |
| FG.B/DZ.AICDA.IGKC | HRK | 0.68 | FG.EC.GSTA2.AADAC | SPINT1 | 0.58 | FG.Fibro.CCL13.CFD | BMP4 | 0.95 |
| FG.B/DZ.AICDA.IGKC | RP11-132N15.3 | 0.67 | FG.EC.GSTA2.AADAC | RAC1 | 0.58 | FG.Fibro.CCL19.C3 | PTGDS | 3.17 |
| FG.B/DZ.AICDA.IGKC | BLK | 0.81 | FG.EC.GSTA2.AADAC | GOLT1A | 0.58 | FG.Fibro.CCL19.C3 | CCL19 | 3.10 |
| FG.B/DZ.AICDA.IGKC | IL4R | 0.77 | FG.EC.GSTA2.AADAC | MXD1 | 0.58 | FG.Fibro.CCL19.C3 | DCN | 2.56 |
| FG.B/DZ.AICDA.IGKC | SWAP70 | 0.86 | FG.EC.GSTA2.AADAC | DPP4 | 0.58 | FG.Fibro.CCL19.C3 | C3 | 2.21 |
| FG.B/DZ.AICDA.IGKC | SLC25A5 | 1.05 | FG.EC.GSTA2.AADAC | CBLC | 0.58 | FG.Fibro.CCL19.C3 | SOD3 | 1.85 |
| FG.B/DZ.AICDA.IGKC | HNRNPA1 | 0.89 | FG.EC.GSTA2.AADAC | AIG1 | 0.58 | FG.Fibro.CCL19.C3 | CTSK | 1.84 |
| FG.B/DZ.AICDA.IGKC | POU2F2 | 0.88 | FG.EC.GSTA2.AADAC | HADH | 0.58 | FG.Fibro.CCL19.C3 | TDO2 | 1.62 |
| FG.B/DZ.AICDA.IGLC2 | CD79B | 2.01 | FG.EC.GSTA2.AADAC | MDH2 | 0.58 | FG.Fibro.CCL19.C3 | C1R | 2.50 |
| FG.B/DZ.AICDA.IGLC2 | TCL1A | 1.97 | FG.EC.GSTA2.AADAC | CRLS1 | 0.58 | FG.Fibro.CCL19.C3 | C1S | 2.63 |
| FG.B/DZ.AICDA.IGLC2 | RGS13 | 1.73 | FG.EC.GSTA2.AADAC | ACTN4 | 0.57 | FG.Fibro.CCL21.CCL19 | CCL21 | 6.00 |
| FG.B/DZ.AICDA.IGLC2 | MARCKSL1 | 1.67 | FG.EC.GSTA2.AADAC | AK3 | 0.57 | FG.Fibro.CCL21.CCL19 | CCL19 | 5.34 |
| FG.B/DZ.AICDA.IGLC2 | NEIL1 | 1.41 | FG.EC.GSTA2.AADAC | TUBB2A | 0.57 | FG.Fibro.CCL21.CCL19 | PTGDS | 3.59 |
| FG.B/DZ.AICDA.IGLC2 | VPREB3 | 1.39 | FG.EC.GSTA2.AADAC | NDUFB5 | 0.57 | FG.Fibro.CCL21.CCL19 | ADH1B | 2.74 |
| FG.B/DZ.AICDA.IGLC2 | IRF8 | 1.30 | FG.EC.GSTA2.AADAC | RIOK3 | 0.57 | FG.Fibro.CCL21.CCL19 | C3 | 2.58 |
| FG.B/DZ.AICDA.IGLC2 | RP11-231C14.7 | 1.28 | FG.EC.GSTA2.AADAC | ACE | 0.57 | FG.Fibro.CCL21.CCL19 | IGFBP5 | 2.28 |
| FG.B/DZ.AICDA.IGLC2 | BIK | 1.24 | FG.EC.GSTA2.AADAC | MPC2 | 0.57 | FG.Fibro.CCL21.CCL19 | DCN | 2.59 |
| FG.B/DZ.AICDA.IGLC2 | LRMP | 1.22 | FG.EC.GSTA2.AADAC | CHMP2A | 0.57 | FG.Fibro.CFD.IGHM | CFD | 3.65 |
| FG.B/DZ.AICDA.IGLC2 | BCAS4 | 1.20 | FG.EC.GSTA2.AADAC | NDUFA10 | 0.57 | FG.Fibro.CFD.IGHM | ADAMDEC1 | 3.35 |
| FG.B/DZ.AICDA.IGLC2 | POU2AF1 | 1.19 | FG.EC.GSTA2.AADAC | C17orf78 | 0.57 | FG.Fibro.CFD.IGHM | APOE | 3.16 |
| FG.B/DZ.AICDA.IGLC2 | SUGCT | 1.17 | FG.EC.GSTA2.AADAC | TIMM13 | 0.57 | FG.Fibro.CFD.IGHM | LUM | 3.11 |
| FG.B/DZ.AICDA.IGLC2 | SERPINA9 | 1.17 | FG.EC.GSTA2.AADAC | A1CF | 0.57 | FG.Fibro.CFD.IGHM | MFAP4 | 2.77 |
| FG.B/DZ.AICDA.IGLC2 | BASP1 | 1.13 | FG.EC.GSTA2.AADAC | TMEM54 | 0.57 | FG.Fibro.CFD.IGHM | RARRES2 | 2.67 |
| FG.B/DZ.AICDA.IGLC2 | SPIB | 1.09 | FG.EC.GSTA2.AADAC | ESPN | 0.57 | FG.Fibro.CFD.IGHM | CXCL14 | 2.63 |
| FG.B/DZ.AICDA.IGLC2 | GGA2 | 1.08 | FG.EC.GSTA2.AADAC | NDUFA9 | 0.57 | FG.Fibro.CFD.IGHM | C1S | 2.58 |
| FG.B/DZ.AICDA.IGLC2 | BCL7A | 1.06 | FG.EC.GSTA2.AADAC | PNP | 0.56 | FG.Fibro.CFD.IGHM | DCN | 2.54 |
| FG.B/DZ.AICDA.IGLC2 | AICDA | 1.03 | FG.EC.GSTA2.AADAC | CCDC25 | 0.56 | FG.Fibro.CFD.IGHM | FBLN1 | 2.52 |
| FG.B/DZ.AICDA.IGLC2 | RFTN1 | 1.03 | FG.EC.GSTA2.AADAC | NDUFB7 | 0.56 | FG.Fibro.CFD.IGHM | CCL11 | 2.50 |
| FG.B/DZ.AICDA.IGLC2 | GCSAM | 0.94 | FG.EC.GSTA2.AADAC | PCSK5 | 0.56 | FG.Fibro.CFD.IGHM | C1R | 2.37 |
| FG.B/DZ.AICDA.IGLC2 | AC023590.1 | 0.94 | FG.EC.GSTA2.AADAC | MIR22HG | 0.56 | FG.Fibro.CFD.IGHM | IFITM3 | 2.36 |
| FG.B/DZ.AICDA.IGLC2 | HRK | 0.65 | FG.EC.GSTA2.AADAC | AC004556.1 | 0.56 | FG.Fibro.CFD.IGHM | A2M | 2.36 |
| FG.B/DZ.AICDA.IGLC2 | RGS16 | 1.16 | FG.EC.GSTA2.AADAC | RHOC | 0.56 | FG.Fibro.CFD.IGHM | COL3A1 | 2.34 |
| FG.B/DZ.AICDA.IGLC2 | LIMD2 | 1.40 | FG.EC.GSTA2.AADAC | PANK3 | 0.56 | FG.Fibro.CFD.IGHM | IGFBP7 | 2.34 |
| FG.B/DZ.AICDA.IGLC2 | P2RX5 | 1.00 | FG.EC.GSTA2.AADAC | EBP | 0.56 | FG.Fibro.CFD.IGHM | COL1A2 | 2.29 |
| FG.B/DZ.AICDA.IGLC2 | HMCES | 1.12 | FG.EC.GSTA2.AADAC | SLC20A1 | 0.56 | FG.Fibro.CFD.IGHM | PPP1R14A | 2.23 |
| FG.B/DZ.TOP2A.UBE2C | HMGB2 | 2.73 | FG.EC.GSTA2.AADAC | PHB2 | 0.56 | FG.Fibro.CFD.IGHM | CTSC | 2.17 |
| FG.B/DZ.TOP2A.UBE2C | HIST1H4C | 2.64 | FG.EC.GSTA2.AADAC | SLC28A2 | 0.55 | FG.Fibro.CFD.IGHM | RBP1 | 2.17 |
| FG.B/DZ.TOP2A.UBE2C | STMN1 | 2.63 | FG.EC.GSTA2.AADAC | ACO2 | 0.55 | FG.Fibro.CFD.IGHM | GGT5 | 2.16 |
| FG.B/DZ.TOP2A.UBE2C | HMGN2 | 2.25 | FG.EC.GSTA2.AADAC | RAB1A | 0.55 | FG.Fibro.CFD.IGHM | CALD1 | 2.12 |
| FG.B/DZ.TOP2A.UBE2C | TUBB | 2.12 | FG.EC.GSTA2.AADAC | FUT2 | 0.55 | FG.Fibro.CFD.IGHM | PTN | 2.11 |
| FG.B/DZ.TOP2A.UBE2C | TUBA1B | 2.07 | FG.EC.GSTA2.AADAC | ACO1 | 0.55 | FG.Fibro.CFD.IGHM | PLTP | 2.11 |
| FG.B/DZ.TOP2A.UBE2C | KIAA0101 | 2.04 | FG.EC.GSTA2.AADAC | PLPP2 | 0.55 | FG.Fibro.CFD.IGHM | TMEM176B | 2.10 |
| FG.B/DZ.TOP2A.UBE2C | H2AFZ | 2.04 | FG.EC.GSTA2.AADAC | SMLR1 | 0.55 | FG.Fibro.CFD.IGHM | CCL2 | 2.07 |
| FG.B/DZ.TOP2A.UBE2C | HMGB1 | 1.94 | FG.EC.GSTA2.AADAC | GOLGA4 | 0.55 | FG.Fibro.CFD.IGHM | ABCA8 | 2.03 |
| FG.B/DZ.TOP2A.UBE2C | UBE2C | 1.91 | FG.EC.GSTA2.AADAC | PLCB3 | 0.54 | FG.Fibro.CFD.IGHM | GPX3 | 2.03 |
| FG.B/DZ.TOP2A.UBE2C | TOP2A | 1.88 | FG.EC.GSTA2.AADAC | SEC13 | 0.54 | FG.Fibro.CFD.IGHM | LGALS1 | 2.00 |
| FG.B/DZ.TOP2A.UBE2C | PTTG1 | 1.88 | FG.EC.GSTA2.AADAC | SLC15A1 | 0.54 | FG.Fibro.CFD.IGHM | MMP2 | 1.99 |
| FG.B/DZ.TOP2A.UBE2C | NUSAP1 | 1.80 | FG.EC.GSTA2.AADAC | NDUFS8 | 0.54 | FG.Fibro.CFD.IGHM | CFH | 1.97 |
| FG.B/DZ.TOP2A.UBE2C | RGS13 | 1.76 | FG.EC.GSTA2.AADAC | PTPRF | 0.54 | FG.Fibro.CFD.IGHM | LTBP4 | 1.95 |
| FG.B/DZ.TOP2A.UBE2C | TCL1A | 1.74 | FG.EC.GSTA2.AADAC | NDUFC1 | 0.54 | FG.Fibro.CFD.IGHM | COL6A2 | 1.89 |
| FG.B/DZ.TOP2A.UBE2C | HMGA1 | 1.71 | FG.EC.GSTA2.AADAC | AC004754.3 | 0.54 | FG.Fibro.CFD.IGHM | HAPLN1 | 1.88 |
| FG.B/DZ.TOP2A.UBE2C | CDK1 | 1.68 | FG.EC.GSTA2.AADAC | FXYD3 | 0.54 | FG.Fibro.CFD.IGHM | CYGB | 1.87 |
| FG.B/DZ.TOP2A.UBE2C | MKI67 | 1.64 | FG.EC.GSTA2.AADAC | AP1S1 | 0.54 | FG.Fibro.CFD.IGHM | PLPP3 | 1.86 |
| FG.B/DZ.TOP2A.UBE2C | H2AFV | 1.60 | FG.EC.GSTA2.AADAC | TNFSF10 | 0.54 | FG.Fibro.CFD.IGHM | CCL8 | 1.84 |
| FG.B/DZ.TOP2A.UBE2C | DUT | 1.59 | FG.EC.GSTA2.AADAC | TOB1 | 0.54 | FG.Fibro.CFD.IGHM | EMILIN1 | 1.83 |
| FG.B/DZ.TOP2A.UBE2C | CENPF | 1.57 | FG.EC.GSTA2.AADAC | ERICH4 | 0.54 | FG.Fibro.CFD.IGHM | CCL13 | 1.83 |
| FG.B/DZ.TOP2A.UBE2C | TK1 | 1.56 | FG.EC.GSTA2.AADAC | LGALS2 | 0.54 | FG.Fibro.CFD.IGHM | SERPING1 | 1.81 |
| FG.B/DZ.TOP2A.UBE2C | HMGN1 | 1.55 | FG.EC.GSTA2.AADAC | ACAA2 | 0.54 | FG.Fibro.CFD.IGHM | GSN | 1.80 |
| FG.B/DZ.TOP2A.UBE2C | GAPDH | 1.55 | FG.EC.GSTA2.AADAC | NDUFB3 | 0.54 | FG.Fibro.CFD.IGHM | QSOX1 | 1.79 |
| FG.B/DZ.TOP2A.UBE2C | RRM2 | 1.53 | FG.EC.GSTA2.AADAC | ARF4 | 0.54 | FG.Fibro.CFD.IGHM | EDIL3 | 1.79 |
| FG.B/DZ.TOP2A.UBE2C | LRMP | 1.51 | FG.EC.GSTA2.AADAC | SLC25A1 | 0.53 | FG.Fibro.CFD.IGHM | CXCL12 | 1.76 |
| FG.B/DZ.TOP2A.UBE2C | SMC4 | 1.49 | FG.EC.GSTA2.AADAC | TMEM120A | 0.53 | FG.Fibro.CFD.IGHM | TMEM176A | 1.75 |
| FG.B/DZ.TOP2A.UBE2C | ANP32B | 1.49 | FG.EC.GSTA2.AADAC | XDH | 0.53 | FG.Fibro.CFD.IGHM | S100A13 | 1.74 |
| FG.B/DZ.TOP2A.UBE2C | BIRC5 | 1.48 | FG.EC.GSTA2.AADAC | SULT1C2 | 0.53 | FG.Fibro.CFD.IGHM | PLAC9 | 1.71 |
| FG.B/DZ.TOP2A.UBE2C | CKS2 | 1.46 | FG.EC.GSTA2.AADAC | TMEM98 | 0.53 | FG.Fibro.CFD.IGHM | SPARC | 1.70 |
| FG.B/DZ.TOP2A.UBE2C | CDKN3 | 1.44 | FG.EC.GSTA2.AADAC | PPIC | 0.53 | FG.Fibro.CFD.IGHM | PLPP1 | 1.69 |
| FG.B/DZ.TOP2A.UBE2C | CCNB2 | 1.42 | FG.EC.GSTA2.AADAC | HACL1 | 0.53 | FG.Fibro.CFD.IGHM | TM4SF1 | 1.69 |
| FG.B/DZ.TOP2A.UBE2C | CKS1B | 1.41 | FG.EC.GSTA2.AADAC | TUBA1C | 0.53 | FG.Fibro.CFD.IGHM | NGFRAP1 | 1.66 |
| FG.B/DZ.TOP2A.UBE2C | CDC20 | 1.40 | FG.EC.GSTA2.AADAC | CAST | 0.53 | FG.Fibro.CFD.IGHM | CD63 | 1.66 |
| FG.B/DZ.TOP2A.UBE2C | DEK | 1.40 | FG.EC.GSTA2.AADAC | NDUFS4 | 0.53 | FG.Fibro.CFD.IGHM | PDLIM1 | 1.64 |
| FG.B/DZ.TOP2A.UBE2C | MARCKSL1 | 1.39 | FG.EC.GSTA2.AADAC | TOM1L1 | 0.53 | FG.Fibro.CFD.IGHM | NBL1 | 1.63 |
| FG.B/DZ.TOP2A.UBE2C | RAN | 1.36 | FG.EC.GSTA2.AADAC | EPS8 | 0.52 | FG.Fibro.CFD.IGHM | LGALS3BP | 1.62 |
| FG.B/DZ.TOP2A.UBE2C | TYMS | 1.36 | FG.EC.GSTA2.AADAC | MAOB | 0.52 | FG.Fibro.CFD.IGHM | NUPR1 | 1.61 |
| FG.B/DZ.TOP2A.UBE2C | CCNB1 | 1.36 | FG.EC.GSTA2.AADAC | CXADR | 0.52 | FG.Fibro.CFD.IGHM | MYLS | 1.58 |
| FG.B/DZ.TOP2A.UBE2C | KPNA2 | 1.35 | FG.EC.GSTA2.AADAC | COMT | 0.52 | FG.Fibro.CFD.IGHM | PTGDS | 1.55 |
| FG.B/DZ.TOP2A.UBE2C | SLC25A5 | 1.35 | FG.EC.GSTA2.AADAC | NDUFA8 | 0.52 | FG.Fibro.CFD.IGHM | CLEC14A | 1.55 |
| FG.B/DZ.TOP2A.UBE2C | RANBP1 | 1.35 | FG.EC.GSTA2.AADAC | LINC01207 | 0.52 | FG.Fibro.CFD.IGHM | SPON2 | 1.54 |
| FG.B/DZ.TOP2A.UBE2C | AURKB | 1.31 | FG.EC.GSTA2.AADAC | MAL2 | 0.52 | FG.Fibro.CFD.IGHM | MEG3 | 1.54 |
| FG.B/DZ.TOP2A.UBE2C | HMCES | 1.29 | FG.EC.GSTA2.AADAC | VDAC2 | 0.52 | FG.Fibro.CFD.IGHM | TIMP1 | 1.54 |
| FG.B/DZ.TOP2A.UBE2C | BIK | 1.27 | FG.EC.GSTA2.AADAC | BPHL | 0.52 | FG.Fibro.CFD.IGHM | TCF21 | 1.53 |
| FG.B/DZ.TOP2A.UBE2C | CENPM | 1.26 | FG.EC.GSTA2.AADAC | AP1M2 | 0.52 | FG.Fibro.CFD.IGHM | SPARCL1 | 1.51 |
| FG.B/DZ.TOP2A.UBE2C | PCNA | 1.26 | FG.EC.GSTA2.AADAC | BRI3 | 0.52 | FG.Fibro.CFD.IGHM | PMP22 | 1.50 |
| FG.B/DZ.TOP2A.UBE2C | METAP2 | 1.25 | FG.EC.GSTA2.AADAC | TMEM141 | 0.52 | FG.Fibro.CFD.IGHM | IGFBP6 | 1.50 |
| FG.B/DZ.TOP2A.UBE2C | PKM | 1.24 | FG.EC.GSTA2.AADAC | TMPRSS4 | 0.52 | FG.Fibro.CFD.IGHM | LINC01082 | 1.48 |
| FG.B/DZ.TOP2A.UBE2C | SLBP | 1.23 | FG.EC.GSTA2.AADAC | CDX2 | 0.52 | FG.Fibro.CFD.IGHM | CLEC11A | 1.46 |
| FG.B/DZ.TOP2A.UBE2C | DCK | 1.22 | FG.EC.GSTA2.AADAC | CD68 | 0.52 | FG.Fibro.CFD.IGHM | PCOLCE | 1.45 |
| FG.B/DZ.TOP2A.UBE2C | PSIP1 | 1.20 | FG.EC.GSTA2.AADAC | ECI2 | 0.52 | FG.Fibro.CFD.IGHM | FHL1 | 1.45 |
| FG.B/DZ.TOP2A.UBE2C | PTMA | 1.17 | FG.EC.GSTA2.AADAC | MOGAT2 | 0.52 | FG.Fibro.CFD.IGHM | COL1A1 | 1.44 |
| FG.B/DZ.TOP2A.UBE2C | MCM7 | 1.16 | FG.EC.GSTA2.AADAC | DHRS1 | 0.52 | FG.Fibro.CFD.IGHM | VIM | 1.41 |
| FG.B/DZ.TOP2A.UBE2C | DNMT1 | 1.16 | FG.EC.GSTA2.AADAC | C6orf132 | 0.51 | FG.Fibro.CFD.IGHM | FXYD1 | 1.40 |
| FG.B/DZ.TOP2A.UBE2C | C12orf75 | 1.15 | FG.EC.GSTA2.AADAC | NDUFB6 | 0.51 | FG.Fibro.CFD.IGHM | SEPP1 | 1.39 |
| FG.B/DZ.TOP2A.UBE2C | TUBB4B | 1.13 | FG.EC.GSTA2.AADAC | YIF1A | 0.51 | FG.Fibro.CFD.IGHM | FBLN5 | 1.38 |
| FG.B/DZ.TOP2A.UBE2C | ARPC2 | 1.13 | FG.EC.GSTA2.AADAC | ASL | 0.51 | FG.Fibro.CFD.IGHM | IFI27 | 1.37 |
| FG.B/DZ.TOP2A.UBE2C | PARP1 | 1.12 | FG.EC.GSTA2.AADAC | HNF4A | 0.51 | FG.Fibro.CFD.IGHM | RAB13 | 1.36 |
| FG.B/DZ.TOP2A.UBE2C | UBE2J1 | 1.12 | FG.EC.GSTA2.AADAC | EPS8L3 | 0.51 | FG.Fibro.CFD.IGHM | PROCR | 1.33 |
| FG.B/DZ.TOP2A.UBE2C | ZWINT | 1.12 | FG.EC.GSTA2.AADAC | GLYCTK | 0.51 | FG.Fibro.CFD.IGHM | TNXB | 1.33 |
| FG.B/DZ.TOP2A.UBE2C | H3F3A | 1.12 | FG.EC.GSTA2.AADAC | ABHD2 | 0.51 | FG.Fibro.CFD.IGHM | TPM2 | 1.32 |
| FG.B/DZ.TOP2A.UBE2C | ACTG1 | 1.12 | FG.EC.GSTA2.AADAC | MYO6 | 0.51 | FG.Fibro.CFD.IGHM | TPPP3 | 1.30 |
| FG.B/DZ.TOP2A.UBE2C | SNRPD1 | 1.11 | FG.EC.GSTA2.AADAC | PGRMC1 | 0.50 | FG.Fibro.CFD.IGHM | VCAM1 | 1.30 |
| FG.B/DZ.TOP2A.UBE2C | TCEA1 | 1.11 | FG.EC.GSTA2.AADAC | RP11-532F12.5 | 0.50 | FG.Fibro.CFD.IGHM | SNAI2 | 1.30 |
| FG.B/DZ.TOP2A.UBE2C | AICDA | 1.11 | FG.EC.GSTA2.AADAC | APOA1 | 0.50 | FG.Fibro.CFD.IGHM | PTMS | 1.29 |
| FG.B/DZ.TOP2A.UBE2C | EAF2 | 1.11 | FG.EC.GSTA2.AADAC | EFHD2 | 0.50 | FG.Fibro.CFD.IGHM | NKX2-3 | 1.29 |
| FG.B/DZ.TOP2A.UBE2C | CBX3 | 1.11 | FG.EC.GSTA2.AADAC | SDHC | 0.50 | FG.Fibro.CFD.IGHM | WFDC1 | 1.27 |
| FG.B/DZ.TOP2A.UBE2C | CFL1 | 1.10 | FG.EC.GSTA2.AADAC | RP11-680F8.1 | 0.50 | FG.Fibro.CFD.IGHM | COX7A1 | 1.27 |
| FG.B/DZ.TOP2A.UBE2C | CD79B | 1.10 | FG.EC.GSTA2.AADAC | ALDH3A2 | 0.50 | FG.Fibro.CFD.IGHM | PRKCDBP | 1.25 |
| FG.B/DZ.TOP2A.UBE2C | DHFR | 1.09 | FG.EC.GSTA2.AADAC | OTOP3 | 0.50 | FG.Fibro.CFD.IGHM | COLEC11 | 1.22 |
| FG.B/DZ.TOP2A.UBE2C | GCSAM | 1.09 | FG.EC.GSTA2.AADAC | PLGRKT | 0.50 | FG.Fibro.CFD.IGHM | APOC1 | 1.20 |
| FG.B/DZ.TOP2A.UBE2C | DDX39A | 1.09 | FG.EC.GSTA2.AADAC | TMEM253 | 0.50 | FG.Fibro.CFD.IGHM | COL6A1 | 1.20 |
| FG.B/DZ.TOP2A.UBE2C | HNRNPA2B1 | 1.08 | FG.EC.GSTA2.AADAC | CCDC47 | 0.50 | FG.Fibro.CFD.IGHM | NID1 | 1.17 |
| FG.B/DZ.TOP2A.UBE2C | CCNA2 | 1.08 | FG.EC.GSTA2.AADAC | DHRS7 | 0.49 | FG.Fibro.CFD.IGHM | VCAN | 1.17 |
| FG.B/DZ.TOP2A.UBE2C | HMMR | 1.08 | FG.EC.GSTA2.AADAC | RAB17 | 0.49 | FG.Fibro.CFD.IGHM | SLC9A3R2 | 1.17 |
| FG.B/DZ.TOP2A.UBE2C | MRPL51 | 1.07 | FG.EC.GSTA2.AADAC | CNN3 | 0.49 | FG.Fibro.CFD.IGHM | IL34 | 1.17 |
| FG.B/DZ.TOP2A.UBE2C | ANP32E | 1.06 | FG.EC.GSTA2.AADAC | AIFM1 | 0.49 | FG.Fibro.CFD.IGHM | ADH1B | 1.16 |
| FG.B/DZ.TOP2A.UBE2C | CARHSP1 | 1.06 | FG.EC.GSTA2.AADAC | GHITM | 0.49 | FG.Fibro.CFD.IGHM | TIMP2 | 1.16 |
| FG.B/DZ.TOP2A.UBE2C | H2AFY | 1.05 | FG.EC.GSTA2.AADAC | SCRN2 | 0.49 | FG.Fibro.CFD.IGHM | BMP5 | 1.15 |
| FG.B/DZ.TOP2A.UBE2C | RPA3 | 1.05 | FG.EC.GSTA2.AADAC | SRI | 0.49 | FG.Fibro.CFD.IGHM | ECM1 | 1.15 |
| FG.B/DZ.TOP2A.UBE2C | UBE2S | 1.04 | FG.EC.GSTA2.AADAC | AAMDC | 0.49 | FG.Fibro.CFD.IGHM | SERPINF1 | 1.14 |
| FG.B/DZ.TOP2A.UBE2C | BCAS4 | 1.04 | FG.EC.GSTA2.AADAC | EPN1 | 0.49 | FG.Fibro.CFD.IGHM | FN1 | 1.13 |
| FG.B/DZ.TOP2A.UBE2C | NUF2 | 1.04 | FG.EC.GSTA2.AADAC | NDUFA2 | 0.49 | FG.Fibro.CFD.IGHM | EFEMP2 | 1.12 |
| FG.B/DZ.TOP2A.UBE2C | BASP1 | 1.03 | FG.EC.GSTA2.AADAC | NQO1 | 0.49 | FG.Fibro.CFD.IGHM | SFTA1P | 1.11 |
| FG.B/DZ.TOP2A.UBE2C | CALM3 | 1.02 | FG.EC.GSTA2.AADAC | ATP8B1 | 0.49 | FG.Fibro.CFD.IGHM | EHD2 | 1.09 |
| FG.B/DZ.TOP2A.UBE2C | HN1 | 1.02 | FG.EC.GSTA2.AADAC | TMC5 | 0.49 | FG.Fibro.CFD.IGHM | ANGPTL1 | 1.09 |
| FG.B/DZ.TOP2A.UBE2C | PPIA | 1.02 | FG.EC.GSTA2.AADAC | AVPI1 | 0.49 | FG.Fibro.CFD.IGHM | DPT | 1.09 |
| FG.B/DZ.TOP2A.UBE2C | CDCA7 | 1.02 | FG.EC.GSTA2.AADAC | TMEM82 | 0.48 | FG.Fibro.CFD.IGHM | ASPN | 1.08 |
| FG.B/DZ.TOP2A.UBE2C | PFN1 | 1.01 | FG.EC.GSTA2.AADAC | SULT1E1 | 0.48 | FG.Fibro.CFD.IGHM | PTCH1 | 1.08 |
| FG.B/DZ.TOP2A.UBE2C | GCHFR | 1.01 | FG.EC.GSTA2.AADAC | RARS | 0.48 | FG.Fibro.CFD.IGHM | COL18A1 | 1.06 |
| FG.B/DZ.TOP2A.UBE2C | GMNN | 1.01 | FG.EC.GSTA2.AADAC | METTL7A | 0.48 | FG.Fibro.CFD.IGHM | COL6A3 | 1.05 |
| FG.B/DZ.TOP2A.UBE2C | MAD2L1 | 1.01 | FG.EC.GSTA2.AADAC | MRPL41 | 0.48 | FG.Fibro.CFD.IGHM | GNG11 | 1.04 |
| FG.B/DZ.TOP2A.UBE2C | RBBP7 | 1.01 | FG.EC.GSTA2.AADAC | TMEM176A | 0.48 | FG.Fibro.CFD.IGHM | MXRA8 | 1.03 |
| FG.B/DZ.TOP2A.UBE2C | CORO1A | 1.00 | FG.EC.GSTA2.AADAC | NDUFS1 | 0.48 | FG.Fibro.CFD.IGHM | CST3 | 1.03 |
| FG.B/DZ.TOP2A.UBE2C | HNRNPA3 | 1.00 | FG.EC.GSTA2.AADAC | HTATIP2 | 0.48 | FG.Fibro.CFD.IGHM | COL15A1 | 1.02 |
| FG.B/DZ.TOP2A.UBE2C | TPX2 | 0.99 | FG.EC.GSTA2.AADAC | SLC35A3 | 0.47 | FG.Fibro.CFD.IGHM | CTSK | 1.02 |
| FG.B/DZ.TOP2A.UBE2C | PLK1 | 0.98 | FG.EC.GSTA2.AADAC | EPB41L3 | 0.47 | FG.Fibro.CFD.IGHM | STMN2 | 1.02 |
| FG.B/DZ.TOP2A.UBE2C | PBK | 0.98 | FG.EC.GSTA2.AADAC | COQ4 | 0.47 | FG.Fibro.CFD.IGHM | DDR2 | 1.01 |
| FG.B/DZ.TOP2A.UBE2C | NUCKS1 | 0.98 | FG.EC.GSTA2.AADAC | CENPV | 0.47 | FG.Fibro.CFD.IGHM | SGCE | 1.01 |
| FG.B/DZ.TOP2A.UBE2C | CDCA3 | 0.97 | FG.EC.GSTA2.AADAC | C1orf115 | 0.47 | FG.Fibro.CFD.IGHM | SOD3 | 1.01 |
| FG.B/DZ.TOP2A.UBE2C | TACC3 | 0.96 | FG.EC.GSTA2.AADAC | FAM162A | 0.47 | FG.Fibro.CFD.IGHM | PDGFRA | 0.99 |
| FG.B/DZ.TOP2A.UBE2C | LMNB1 | 0.96 | FG.EC.GSTA2.AADAC | AURKAIP1 | 0.47 | FG.Fibro.CFD.IGHM | NDN | 0.99 |
| FG.B/DZ.TOP2A.UBE2C | CENPU | 0.96 | FG.EC.GSTA2.AADAC | WASL | 0.47 | FG.Fibro.CFD.IGHM | LOXL1 | 0.99 |
| FG.B/DZ.TOP2A.UBE2C | ACTB | 0.95 | FG.EC.GSTA2.AADAC | COMTD1 | 0.47 | FG.Fibro.CFD.IGHM | FSTL1 | 0.97 |
| FG.B/DZ.TOP2A.UBE2C | GINS2 | 0.95 | FG.EC.GSTA2.AADAC | GGT6 | 0.47 | FG.Fibro.CFD.IGHM | AEBP1 | 0.97 |
| FG.B/DZ.TOP2A.UBE2C | AURKA | 0.95 | FG.EC.GSTA2.AADAC | SLC34A2 | 0.47 | FG.Fibro.CFD.IGHM | FOXF1 | 0.97 |
| FG.B/DZ.TOP2A.UBE2C | TFDP1 | 0.95 | FG.EC.GSTA2.AADAC | TSPAN3 | 0.47 | FG.Fibro.CFD.IGHM | CRISPLD2 | 0.93 |
| FG.B/DZ.TOP2A.UBE2C | LDHA | 0.95 | FG.EC.GSTA2.AADAC | ANKRD9 | 0.47 | FG.Fibro.CFD.IGHM | C2 | 0.93 |
| FG.B/DZ.TOP2A.UBE2C | ERH | 0.95 | FG.EC.GSTA2.AADAC | HSD17B4 | 0.47 | FG.Fibro.CFD.IGHM | GSTM5 | 0.93 |
| FG.B/DZ.TOP2A.UBE2C | IRF8 | 0.95 | FG.EC.GSTA2.AADAC | CFTR | 0.46 | FG.Fibro.CFD.IGHM | DKK3 | 0.93 |
| FG.B/DZ.TOP2A.UBE2C | PPP1CA | 0.95 | FG.EC.GSTA2.AADAC | MLEC | 0.46 | FG.Fibro.CFD.IGHM | PLEKHH2 | 0.92 |
| FG.B/DZ.TOP2A.UBE2C | ARPC5 | 0.95 | FG.EC.GSTA2.AADAC | TPMT | 0.46 | FG.Fibro.CFD.IGHM | SCARA5 | 0.91 |
| FG.B/DZ.TOP2A.UBE2C | ASF1B | 0.95 | FG.EC.GSTA2.AADAC | CCND2 | 0.46 | FG.Fibro.CFD.IGHM | HOXC9 | 0.90 |
| FG.B/DZ.TOP2A.UBE2C | SRSF3 | 0.95 | FG.EC.GSTA2.AADAC | CTNND1 | 0.46 | FG.Fibro.CFD.IGHM | PCDH18 | 0.87 |
| FG.B/DZ.TOP2A.UBE2C | TMPO | 0.94 | FG.EC.GSTA2.AADAC | MPC1 | 0.46 | FG.Fibro.CFD.IGHM | MAMDC2 | 0.86 |
| FG.B/DZ.TOP2A.UBE2C | SNRPB | 0.94 | FG.EC.GSTA2.AADAC | RRBP1 | 0.46 | FG.Fibro.CFD.IGHM | ALDH1A3 | 0.86 |
| FG.B/DZ.TOP2A.UBE2C | YBX1 | 0.94 | FG.EC.GSTA2.AADAC | ST14 | 0.46 | FG.Fibro.CFD.IGHM | FXYD6 | 0.85 |
| FG.B/DZ.TOP2A.UBE2C | RMI2 | 0.94 | FG.EC.GSTA2.AADAC | BTNL8 | 0.46 | FG.Fibro.CFD.IGHM | EMID1 | 0.83 |
| FG.B/DZ.TOP2A.UBE2C | SMC2 | 0.93 | FG.EC.GSTA2.AADAC | ATOX1 | 0.46 | FG.Fibro.CFD.IGHM | PAMR1 | 0.80 |
| FG.B/DZ.TOP2A.UBE2C | BCL7A | 0.93 | FG.EC.GSTA2.AADAC | ECI1 | 0.46 | FG.Fibro.CFD.IGHM | FKBP10 | 0.79 |
| FG.B/DZ.TOP2A.UBE2C | FEN1 | 0.93 | FG.EC.GSTA2.AADAC | GMDS | 0.46 | FG.Fibro.CFD.IGHM | DIO2 | 0.78 |
| FG.B/DZ.TOP2A.UBE2C | POUZAF1 | 0.93 | FG.EC.GSTA2.AADAC | PPA2 | 0.46 | FG.Fibro.CFD.IGHM | LOXL4 | 0.66 |
| FG.B/DZ.TOP2A.UBE2C | SMARCB1 | 0.92 | FG.EC.GSTA2.AADAC | FAHD1 | 0.46 | FG.Fibro.CFD.IGHM | LAMA4 | 0.90 |
| FG.B/DZ.TOP2A.UBE2C | ARPC3 | 0.92 | FG.EC.GSTA2.AADAC | POR | 0.46 | FG.Fibro.CFD.IGHM | MXRA5 | 0.83 |
| FG.B/DZ.TOP2A.UBE2C | HNRNPA1 | 0.92 | FG.EC.GSTA2.AADAC | ANKRD37 | 0.46 | FG.Fibro.CFD.IGHM | AKAP12 | 0.85 |
| FG.B/DZ.TOP2A.UBE2C | ACTR3 | 0.91 | FG.EC.GSTA2.AADAC | APLP2 | 0.46 | FG.Fibro.CFD.IGHM | KCNS3 | 0.81 |
| FG.B/DZ.TOP2A.UBE2C | ITGAE | 0.91 | FG.EC.GSTA2.AADAC | TMEM220 | 0.46 | FG.Fibro.CFD.IGHM | TSPAN4 | 1.02 |
| FG.B/DZ.TOP2A.UBE2C | DBI | 0.91 | FG.EC.GSTA2.AADAC | FAM3D | 0.45 | FG.Fibro.CFD.IGHM | RAB34 | 0.97 |
| FG.B/DZ.TOP2A.UBE2C | SERF2 | 0.91 | FG.EC.GSTA2.AADAC | SSX2IP | 0.45 | FG.Fibro.CFD.IGHM | THY1 | 0.88 |
| FG.B/DZ.TOP2A.UBE2C | RAD21 | 0.91 | FG.EC.GSTA2.AADAC | DDAH1 | 0.45 | FG.Fibro.CFD.IGHM | OLFML3 | 0.92 |
| FG.B/DZ.TOP2A.UBE2C | CENPA | 0.90 | FG.EC.GSTA2.AADAC | PDHA1 | 0.45 | FG.Fibro.CFD.IGHM | GPC3 | 0.75 |
| FG.B/DZ.TOP2A.UBE2C | SNRNP25 | 0.90 | FG.EC.GSTA2.AADAC | XPNPEP2 | 0.45 | FG.Fibro.FIGF.EDNRB | CXCL14 | 3.76 |
| FG.B/DZ.TOP2A.UBE2C | BUB3 | 0.90 | FG.EC.GSTA2.AADAC | GUCY2C | 0.45 | FG.Fibro.FIGF.EDNRB | TAGLN | 3.15 |
| FG.B/DZ.TOP2A.UBE2C | KIFC1 | 0.90 | FG.EC.GSTA2.AADAC | MGAM | 0.45 | FG.Fibro.FIGF.EDNRB | CALD1 | 3.01 |
| FG.B/DZ.TOP2A.UBE2C | SET | 0.89 | FG.EC.GSTA2.AADAC | TMEM30B | 0.45 | FG.Fibro.FIGF.EDNRB | EDNRB | 3.00 |
| FG.B/DZ.TOP2A.UBE2C | GGA2 | 0.89 | FG.EC.GSTA2.AADAC | PLAC8 | 0.45 | FG.Fibro.FIGF.EDNRB | ACTA2 | 2.95 |
| FG.B/DZ.TOP2A.UBE2C | DLGAP5 | 0.89 | FG.EC.GSTA2.AADAC | GOLM1 | 0.45 | FG.Fibro.FIGF.EDNRB | COL6A2 | 2.94 |
| FG.B/DZ.TOP2A.UBE2C | UBEZT | 0.89 | FG.EC.GSTA2.AADAC | IFT172 | 0.44 | FG.Fibro.FIGF.EDNRB | POSTN | 2.82 |
| FG.B/DZ.TOP2A.UBE2C | PRDX3 | 0.89 | FG.EC.GSTA2.AADAC | ME1 | 0.44 | FG.Fibro.FIGF.EDNRB | IGFBP7 | 2.76 |
| FG.B/DZ.TOP2A.UBE2C | SNRPG | 0.88 | FG.EC.GSTA2.AADAC | GPI | 0.44 | FG.Fibro.FIGF.EDNRB | COL6A1 | 2.70 |
| FG.B/DZ.TOP2A.UBE2C | CBX5 | 0.87 | FG.EC.GSTA2.AADAC | TUFM | 0.44 | FG.Fibro.FIGF.EDNRB | LGALS1 | 2.62 |
| FG.B/DZ.TOP2A.UBE2C | CDT1 | 0.87 | FG.EC.GSTA2.AADAC | HN1 | 0.44 | FG.Fibro.FIGF.EDNRB | FRZB | 2.60 |
| FG.B/DZ.TOP2A.UBE2C | SRSF9 | 0.87 | FG.EC.GSTA2.AADAC | MIR194-2HG | 0.44 | FG.Fibro.FIGF.EDNRB | APOD | 2.55 |
| FG.B/DZ.TOP2A.UBE2C | SUMO2 | 0.86 | FG.EC.GSTA2.AADAC | SDHD | 0.44 | FG.Fibro.FIGF.EDNRB | IGFBP5 | 2.47 |
| FG.B/DZ.TOP2A.UBE2C | ATP5L | 0.86 | FG.EC.GSTA2.AADAC | MS4A10 | 0.44 | FG.Fibro.FIGF.EDNRB | NBL1 | 2.40 |
| FG.B/DZ.TOP2A.UBE2C | MCM3 | 0.86 | FG.EC.GSTA2.AADAC | VIMP | 0.44 | FG.Fibro.FIGF.EDNRB | CTGF | 2.38 |
| FG.B/DZ.TOP2A.UBE2C | SPC25 | 0.86 | FG.EC.GSTA2.AADAC | ACOT11 | 0.44 | FG.Fibro.FIGF.EDNRB | MMP2 | 2.37 |
| FG.B/DZ.TOP2A.UBE2C | RFC1 | 0.85 | FG.EC.GSTA2.AADAC | MRPS25 | 0.44 | FG.Fibro.FIGF.EDNRB | COL1A2 | 2.37 |
| FG.B/DZ.TOP2A.UBE2C | CDCA8 | 0.85 | FG.EC.GSTA2.AADAC | MDH1 | 0.44 | FG.Fibro.FIGF.EDNRB | COL3A1 | 2.34 |
| FG.B/DZ.TOP2A.UBE2C | LSM5 | 0.85 | FG.EC.GSTA2.AADAC | OSGIN1 | 0.44 | FG.Fibro.FIGF.EDNRB | F3 | 2.32 |
| FG.B/DZ.TOP2A.UBE2C | NPM1 | 0.85 | FG.EC.GSTA2.AADAC | MRPS33 | 0.44 | FG.Fibro.FIGF.EDNRB | TPM2 | 2.32 |
| FG.B/DZ.TOP2A.UBE2C | PPP1CC | 0.85 | FG.EC.GSTA2.AADAC | TREH | 0.44 | FG.Fibro.FIGF.EDNRB | PDGFRA | 2.28 |
| FG.B/DZ.TOP2A.UBE2C | SNRPE | 0.85 | FG.EC.GSTA2.AADAC | ARHGDIG | 0.44 | FG.Fibro.FIGF.EDNRB | COL1A1 | 2.23 |
| FG.B/DZ.TOP2A.UBE2C | KIF22 | 0.84 | FG.EC.GSTA2.AADAC | ABHD11 | 0.43 | FG.Fibro.FIGF.EDNRB | SPARC | 2.22 |
| FG.B/DZ.TOP2A.UBE2C | IMP4 | 0.84 | FG.EC.GSTA2.AADAC | APOBEC1 | 0.43 | FG.Fibro.FIGF.EDNRB | BMP4 | 2.21 |
| FG.B/DZ.TOP2A.UBE2C | DNAJC9 | 0.84 | FG.EC.GSTA2.AADAC | SLC31A1 | 0.43 | FG.Fibro.FIGF.EDNRB | CPM | 2.19 |
| FG.B/DZ.TOP2A.UBE2C | MYBL2 | 0.84 | FG.EC.GSTA2.AADAC | PSMA5 | 0.43 | FG.Fibro.FIGF.EDNRB | CAV1 | 2.18 |
| FG.B/DZ.TOP2A.UBE2C | AP3S1 | 0.84 | FG.EC.GSTA2.AADAC | RBKS | 0.43 | FG.Fibro.FIGF.EDNRB | TMEM176B | 2.10 |
| FG.B/DZ.TOP2A.UBE2C | HNRNPD | 0.84 | FG.EC.GSTA2.AADAC | MARVELD3 | 0.43 | FG.Fibro.FIGF.EDNRB | C15 | 2.09 |
| FG.B/DZ.TOP2A.UBE2C | NEIL1 | 0.84 | FG.EC.GSTA2.AADAC | CKMT1B | 0.43 | FG.Fibro.FIGF.EDNRB | MYL9 | 2.07 |
| FG.B/DZ.TOP2A.UBE2C | HMGB3 | 0.83 | FG.EC.GSTA2.AADAC | SLC40A1 | 0.43 | FG.Fibro.FIGF.EDNRB | NPY | 2.04 |
| FG.B/DZ.TOP2A.UBE2C | YWHAH | 0.83 | FG.EC.GSTA2.AADAC | APP | 0.43 | FG.Fibro.FIGF.EDNRB | PLAT | 2.04 |
| FG.B/DZ.TOP2A.UBE2C | RGS16 | 0.83 | FG.EC.GSTA2.AADAC | PON2 | 0.43 | FG.Fibro.FIGF.EDNRB | RARRES2 | 2.00 |
| FG.B/DZ.TOP2A.UBE2C | ASPM | 0.83 | FG.EC.GSTA2.AADAC | CPT1A | 0.43 | FG.Fibro.FIGF.EDNRB | DMKN | 1.98 |
| FG.B/DZ.TOP2A.UBE2C | NANS | 0.83 | FG.EC.GSTA2.AADAC | HDLBP | 0.43 | FG.Fibro.FIGF.EDNRB | C1R | 1.95 |
| FG.B/DZ.TOP2A.UBE2C | CENPW | 0.83 | FG.EC.GSTA2.AADAC | VPS35 | 0.43 | FG.Fibro.FIGF.EDNRB | SPARCL1 | 1.94 |
| FG.B/DZ.TOP2A.UBE2C | RFTN1 | 0.83 | FG.EC.GSTA2.AADAC | TMEM219 | 0.43 | FG.Fibro.FIGF.EDNRB | IFITM3 | 1.92 |
| FG.B/DZ.TOP2A.UBE2C | DAAM1 | 0.82 | FG.EC.GSTA2.AADAC | TMPRSS2 | 0.43 | FG.Fibro.FIGF.EDNRB | MFAP4 | 1.89 |
| FG.B/DZ.TOP2A.UBE2C | LSM8 | 0.82 | FG.EC.GSTA2.AADAC | SLC30A1 | 0.42 | FG.Fibro.FIGF.EDNRB | SDC2 | 1.85 |
| FG.B/DZ.TOP2A.UBE2C | UCP2 | 0.82 | FG.EC.GSTA2.AADAC | CFDP1 | 0.42 | FG.Fibro.FIGF.EDNRB | IGFBP6 | 1.85 |
| FG.B/DZ.TOP2A.UBE2C | CLSPN | 0.82 | FG.EC.GSTA2.AADAC | ACADSB | 0.42 | FG.Fibro.FIGF.EDNRB | TMEM119 | 1.84 |
| FG.B/DZ.TOP2A.UBE2C | LCP1 | 0.82 | FG.EC.GSTA2.AADAC | FAM96A | 0.42 | FG.Fibro.FIGF.EDNRB | TGFBI | 1.81 |
| FG.B/DZ.TOP2A.UBE2C | SKA2 | 0.82 | FG.EC.GSTA2.AADAC | RNPEP | 0.42 | FG.Fibro.FIGF.EDNRB | MMP11 | 1.79 |
| FG.B/DZ.TOP2A.UBE2C | MCM5 | 0.82 | FG.EC.GSTA2.AADAC | CHP1 | 0.42 | FG.Fibro.FIGF.EDNRB | LAMA4 | 1.79 |
| FG.B/DZ.TOP2A.UBE2C | SERPINA9 | 0.82 | FG.EC.GSTA2.AADAC | ADTRP | 0.42 | FG.Fibro.FIGF.EDNRB | NUPR1 | 1.76 |
| FG.B/DZ.TOP2A.UBE2C | MND1 | 0.81 | FG.EC.GSTA2.AADAC | CKMT1A | 0.42 | FG.Fibro.FIGF.EDNRB | GPX3 | 1.76 |
| FG.B/DZ.TOP2A.UBE2C | SWAP70 | 0.81 | FG.EC.GSTA2.AADAC | TCEA3 | 0.42 | FG.Fibro.FIGF.EDNRB | ITGB1 | 1.74 |
| FG.B/DZ.TOP2A.UBE2C | RBBP4 | 0.81 | FG.EC.GSTA2.AADAC | PLEK2 | 0.42 | FG.Fibro.FIGF.EDNRB | FIGF | 1.73 |
| FG.B/DZ.TOP2A.UBE2C | TPI1 | 0.81 | FG.EC.GSTA2.AADAC | TMEM25 | 0.42 | FG.Fibro.FIGF.EDNRB | TSHZ2 | 1.70 |
| FG.B/DZ.TOP2A.UBE2C | GTSE1 | 0.81 | FG.EC.GSTA2.AADAC | CCNG1 | 0.42 | FG.Fibro.FIGF.EDNRB | ACTG2 | 1.68 |
| FG.B/DZ.TOP2A.UBE2C | MZT2B | 0.81 | FG.EC.GSTA2.AADAC | CCND1 | 0.42 | FG.Fibro.FIGF.EDNRB | AGT | 1.67 |
| FG.B/DZ.TOP2A.UBE2C | HDAC1 | 0.80 | FG.EC.GSTA2.AADAC | TUBB4B | 0.42 | FG.Fibro.FIGF.EDNRB | S100A13 | 1.65 |
| FG.B/DZ.TOP2A.UBE2C | CCDC167 | 0.80 | FG.EC.GSTA2.AADAC | TOR1AIP2 | 0.42 | FG.Fibro.FIGF.EDNRB | EMILIN1 | 1.65 |
| FG.B/DZ.TOP2A.UBE2C | TUBB2A | 0.80 | FG.EC.GSTA2.AADAC | C19orf70 | 0.42 | FG.Fibro.FIGF.EDNRB | NSG1 | 1.65 |
| FG.B/DZ.TOP2A.UBE2C | H2AFX | 0.80 | FG.EC.GSTA2.AADAC | CDHR2 | 0.42 | FG.Fibro.FIGF.EDNRB | SPON2 | 1.63 |
| FG.B/DZ.TOP2A.UBE2C | CENPE | 0.80 | FG.EC.GSTA2.AADAC | SLC16A10 | 0.42 | FG.Fibro.FIGF.EDNRB | DPT | 1.62 |
| FG.B/DZ.TOP2A.UBE2C | YWHAE | 0.80 | FG.EC.GSTA2.AADAC | PDZK1 | 0.42 | FG.Fibro.FIGF.EDNRB | BMP5 | 1.62 |
| FG.B/DZ.TOP2A.UBE2C | LIMD2 | 0.80 | FG.EC.GSTA2.AADAC | SNX6 | 0.42 | FG.Fibro.FIGF.EDNRB | CYGB | 1.61 |
| FG.B/DZ.TOP2A.UBE2C | USP1 | 0.80 | FG.EC.GSTA2.AADAC | TLCD2 | 0.41 | FG.Fibro.FIGF.EDNRB | COL12A1 | 1.61 |
| FG.B/DZ.TOP2A.UBE2C | SGOL2 | 0.79 | FG.EC.GSTA2.AADAC | PLEKHJ1 | 0.41 | FG.Fibro.FIGF.EDNRB | FHL1 | 1.60 |
| FG.B/DZ.TOP2A.UBE2C | HNRNPC | 0.79 | FG.EC.GSTA2.AADAC | DBP | 0.41 | FG.Fibro.FIGF.EDNRB | LTBP4 | 1.57 |
| FG.B/DZ.TOP2A.UBE2C | GDI2 | 0.79 | FG.EC.GSTA2.AADAC | LPCAT3 | 0.41 | FG.Fibro.FIGF.EDNRB | COX7A1 | 1.56 |
| FG.B/DZ.TOP2A.UBE2C | LBR | 0.78 | FG.EC.GSTA2.AADAC | WEE1 | 0.41 | FG.Fibro.FIGF.EDNRB | SOX6 | 1.53 |
| FG.B/DZ.TOP2A.UBE2C | EZH2 | 0.78 | FG.EC.GSTA2.AADAC | TJP2 | 0.41 | FG.Fibro.FIGF.EDNRB | PDGFD | 1.51 |
| FG.B/DZ.TOP2A.UBE2C | TPD52 | 0.78 | FG.EC.GSTA2.AADAC | IRF6 | 0.41 | FG.Fibro.FIGF.EDNRB | TNC | 1.49 |
| FG.B/DZ.TOP2A.UBE2C | SMIM14 | 0.78 | FG.EC.GSTA2.AADAC | ITGA6 | 0.41 | FG.Fibro.FIGF.EDNRB | SULF1 | 1.49 |
| FG.B/DZ.TOP2A.UBE2C | S1PR2 | 0.78 | FG.EC.GSTA2.AADAC | SYNJ2BP | 0.41 | FG.Fibro.FIGF.EDNRB | A2M | 1.48 |
| FG.B/DZ.TOP2A.UBE2C | OAZ1 | 0.78 | FG.EC.GSTA2.AADAC | ABO | 0.41 | FG.Fibro.FIGF.EDNRB | SOD3 | 1.45 |
| FG.B/DZ.TOP2A.UBE2C | CCND3 | 0.77 | FG.EC.GSTA2.AADAC | CYP4F12 | 0.41 | FG.Fibro.FIGF.EDNRB | COL6A3 | 1.44 |
| FG.B/DZ.TOP2A.UBE2C | SGOL1 | 0.77 | FG.EC.GSTA2.AADAC | CALML4 | 0.41 | FG.Fibro.FIGF.EDNRB | LIMCH1 | 1.43 |
| FG.B/DZ.TOP2A.UBE2C | SRSF10 | 0.77 | FG.EC.GSTA2.AADAC | MAP2K2 | 0.41 | FG.Fibro.FIGF.EDNRB | OLFML3 | 1.42 |
| FG.B/DZ.TOP2A.UBE2C | YWHAQ | 0.77 | FG.EC.GSTA2.AADAC | ADGRG7 | 0.41 | FG.Fibro.FIGF.EDNRB | FOXF1 | 1.41 |
| FG.B/DZ.TOP2A.UBE2C | TUBB2B | 0.77 | FG.EC.GSTA2.AADAC | 2-Mar | 0.41 | FG.Fibro.FIGF.EDNRB | FENDRR | 1.38 |
| FG.B/DZ.TOP2A.UBE2C | PSMA4 | 0.76 | FG.EC.GSTA2.AADAC | KDELR2 | 0.41 | FG.Fibro.FIGF.EDNRB | CFH | 1.36 |
| FG.B/DZ.TOP2A.UBE2C | CALM2 | 0.76 | FG.EC.GSTA2.AADAC | ARHGAP18 | 0.41 | FG.Fibro.FIGF.EDNRB | ABI3BP | 1.34 |
| FG.B/DZ.TOP2A.UBE2C | CD81 | 0.76 | FG.EC.GSTA2.AADAC | HSDL2 | 0.41 | FG.Fibro.FIGF.EDNRB | PCOLCE | 1.34 |
| FG.B/DZ.TOP2A.UBE2C | COX8A | 0.76 | FG.EC.GSTA2.AADAC | FAM234A | 0.41 | FG.Fibro.FIGF.EDNRB | COL4A2 | 1.33 |
| FG.B/DZ.TOP2A.UBE2C | MPC2 | 0.76 | FG.EC.GSTA2.AADAC | ACADM | 0.41 | FG.Fibro.FIGF.EDNRB | TGFB1I1 | 1.31 |
| FG.B/DZ.TOP2A.UBE2C | RPL39L | 0.76 | FG.EC.GSTA2.AADAC | ECHDC2 | 0.40 | FG.Fibro.FIGF.EDNRB | COL5A1 | 1.30 |
| FG.B/DZ.TOP2A.UBE2C | RRM1 | 0.76 | FG.EC.GSTA2.AADAC | SLC7A7 | 0.40 | FG.Fibro.FIGF.EDNRB | HTRA1 | 1.30 |
| FG.B/DZ.TOP2A.UBE2C | PLEKHF2 | 0.76 | FG.EC.GSTA2.AADAC | QPRT | 0.40 | FG.Fibro.FIGF.EDNRB | EFEMP2 | 1.28 |
| FG.B/DZ.TOP2A.UBE2C | KHDRBS1 | 0.76 | FG.EC.GSTA2.AADAC | ETFDH | 0.40 | FG.Fibro.FIGF.EDNRB | TRPA1 | 1.27 |
| FG.B/DZ.TOP2A.UBE2C | C19orf48 | 0.76 | FG.EC.GSTA2.AADAC | SBDS | 0.40 | FG.Fibro.FIGF.EDNRB | ENHO | 1.27 |
| FG.B/DZ.TOP2A.UBE2C | RP11-231C14.7 | 0.76 | FG.EC.GSTA2.AADAC | TP53I3 | 0.40 | FG.Fibro.FIGF.EDNRB | LY6H | 1.23 |
| FG.B/DZ.TOP2A.UBE2C | PRC1 | 0.75 | FG.EC.GSTA2.AADAC | BAIAP2L2 | 0.40 | FG.Fibro.FIGF.EDNRB | MXRA8 | 1.23 |
| FG.B/DZ.TOP2A.UBE2C | BRI3BP | 0.75 | FG.EC.GSTA2.AADAC | BTNL3 | 0.40 | FG.Fibro.FIGF.EDNRB | NDUFA4L2 | 1.22 |
| FG.B/DZ.TOP2A.UBE2C | SMS | 0.75 | FG.EC.GSTA2.AADAC | USP2 | 0.40 | FG.Fibro.FIGF.EDNRB | PCSK6 | 1.19 |
| FG.B/DZ.TOP2A.UBE2C | HIST1H1D | 0.75 | FG.EC.GSTA2.AADAC | GOT1 | 0.40 | FG.Fibro.FIGF.EDNRB | FAM150B | 1.15 |
| FG.B/DZ.TOP2A.UBE2C | NDC80 | 0.75 | FG.EC.GSTA2.AADAC | AIMP2 | 0.40 | FG.Fibro.FIGF.EDNRB | CDH13 | 1.11 |
| FG.B/DZ.TOP2A.UBE2C | FBXO5 | 0.75 | FG.EC.GSTA2.AADAC | ILVBL | 0.40 | FG.Fibro.FIGF.EDNRB | ECM1 | 1.11 |
| FG.B/DZ.TOP2A.UBE2C | DTYMK | 0.74 | FG.EC.GSTA2.AADAC | LUZP1 | 0.40 | FG.Fibro.FIGF.EDNRB | COL4A5 | 1.05 |
| FG.B/DZ.TOP2A.UBE2C | CKAP2 | 0.74 | FG.EC.GSTA2.AADAC | SLC35B1 | 0.40 | FG.Fibro.FIGF.EDNRB | VSTM2A | 0.93 |
| FG.B/DZ.TOP2A.UBE2C | SRP9 | 0.74 | FG.EC.GSTA2.AADAC | ZNF593 | 0.40 | FG.Fibro.FIGF.EDNRB | CLEC11A | 1.36 |
| FG.B/DZ.TOP2A.UBE2C | LSM6 | 0.74 | FG.EC.GSTA2.AADAC | ADH6 | 0.40 | FG.Fibro.FIGF.EDNRB | LHFP | 1.17 |
| FG.B/DZ.TOP2A.UBE2C | MBD4 | 0.74 | FG.EC.GSTA2.AADAC | SLC44A4 | 0.40 | FG.Fibro.FIGF.EDNRB | PDE1A | 0.70 |
| FG.B/DZ.TOP2A.UBE2C | EZR | 0.74 | FG.EC.GSTA2.AADAC | CDKN1A | 0.40 | FG.Fibro.FIGF.EDNRB | EMID1 | 1.07 |
| FG.B/DZ.TOP2A.UBE2C | CHCHD2 | 0.74 | FG.EC.GSTA2.AADAC | FLNB | 0.40 | FG.Fibro.FIGF.EDNRB | RP11-449D8.1 | 0.69 |
| FG.B/DZ.TOP2A.UBE2C | NCAPG | 0.74 | FG.EC.GSTA2.AADAC | RAB6A | 0.40 | FG.Fibro.FIGF.EDNRB | SGCE | 1.34 |
| FG.B/DZ.TOP2A.UBE2C | CCT8 | 0.74 | FG.EC.GSTA2.AADAC | SLC6A20 | 0.40 | FG.Fibro.IFITM1.ADAMDEC1 | ADAMDEC1 | 3.92 |
| FG.B/DZ.TOP2A.UBE2C | CENPH | 0.73 | FG.EC.GSTA2.AADAC | H2AFJ | 0.40 | FG.Fibro.IFITM1.ADAMDEC1 | LUM | 3.55 |
| FG.B/DZ.TOP2A.UBE2C | HNRNPM | 0.73 | FG.EC.GSTA2.AADAC | MSRB2 | 0.40 | FG.Fibro.IFITM1.ADAMDEC1 | CXCL14 | 3.46 |
| FG.B/DZ.TOP2A.UBE2C | ODC1 | 0.73 | FG.EC.GSTA2.AADAC | GBA2 | 0.40 | FG.Fibro.IFITM1.ADAMDEC1 | CCL11 | 3.22 |
| FG.B/DZ.TOP2A.UBE2C | UBE2G1 | 0.73 | FG.EC.GSTA2.AADAC | SLIRP | 0.40 | FG.Fibro.IFITM1.ADAMDEC1 | DCN | 3.21 |
| FG.B/DZ.TOP2A.UBE2C | HELLS | 0.73 | FG.EC.GSTA2.AADAC | GALE | 0.40 | FG.Fibro.IFITM1.ADAMDEC1 | CFH | 3.13 |
| FG.B/DZ.TOP2A.UBE2C | NUDT21 | 0.72 | FG.EC.GSTA2.AADAC | FAT1 | 0.39 | FG.Fibro.IFITM1.ADAMDEC1 | GPX3 | 2.86 |
| FG.B/DZ.TOP2A.UBE2C | SMARCA4 | 0.72 | FG.EC.GSTA2.AADAC | PBX1 | 0.39 | FG.Fibro.IFITM1.ADAMDEC1 | MFAP4 | 2.83 |
| FG.B/DZ.TOP2A.UBE2C | MOB1A | 0.72 | FG.EC.GSTA2.AADAC | DHRS4-AS1 | 0.39 | FG.Fibro.IFITM1.ADAMDEC1 | CCL8 | 2.70 |
| FG.B/DZ.TOP2A.UBE2C | GRHPR | 0.72 | FG.EC.GSTA2.AADAC | PPARA | 0.39 | FG.Fibro.IFITM1.ADAMDEC1 | APOE | 2.62 |
| FG.B/DZ.TOP2A.UBE2C | PLIN3 | 0.72 | FG.EC.GSTA2.AADAC | CTNNA1 | 0.39 | FG.Fibro.IFITM1.ADAMDEC1 | C1S | 2.54 |
| FG.B/DZ.TOP2A.UBE2C | RHOA | 0.72 | FG.EC.GSTA2.AADAC | ZNHIT1 | 0.39 | FG.Fibro.IFITM1.ADAMDEC1 | SPARCL1 | 2.50 |
| FG.B/DZ.TOP2A.UBE2C | STX7 | 0.72 | FG.EC.GSTA2.AADAC | PDE4C | 0.39 | FG.Fibro.IFITM1.ADAMDEC1 | CALD1 | 2.49 |
| FG.B/DZ.TOP2A.UBE2C | PTPN18 | 0.72 | FG.EC.GSTA2.AADAC | ALPI | 0.39 | FG.Fibro.IFITM1.ADAMDEC1 | ABCA8 | 2.43 |
| FG.B/DZ.TOP2A.UBE2C | COX5A | 0.71 | FG.EC.GSTA2.AADAC | TMEM236 | 0.39 | FG.Fibro.IFITM1.ADAMDEC1 | CCL2 | 2.43 |
| FG.B/DZ.TOP2A.UBE2C | ANAPC11 | 0.71 | FG.EC.GSTA2.AADAC | MRPS12 | 0.39 | FG.Fibro.IFITM1.ADAMDEC1 | C1R | 2.42 |
| FG.B/DZ.TOP2A.UBE2C | HNRNPF | 0.71 | FG.EC.GSTA2.AADAC | ID3 | 0.39 | FG.Fibro.IFITM1.ADAMDEC1 | COL3A1 | 2.41 |
| FG.B/DZ.TOP2A.UBE2C | VRK1 | 0.71 | FG.EC.GSTA2.AADAC | MAPK3 | 0.39 | FG.Fibro.IFITM1.ADAMDEC1 | RARRES2 | 2.40 |
| FG.B/DZ.TOP2A.UBE2C | P2RX5 | 0.71 | FG.EC.GSTA2.AADAC | MRPS34 | 0.39 | FG.Fibro.IFITM1.ADAMDEC1 | IGFBP7 | 2.34 |
| FG.B/DZ.TOP2A.UBE2C | LSM4 | 0.71 | FG.EC.GSTA2.AADAC | NUCB1 | 0.39 | FG.Fibro.IFITM1.ADAMDEC1 | CFD | 2.33 |
| FG.B/DZ.TOP2A.UBE2C | NAA38 | 0.71 | FG.EC.GSTA2.AADAC | ALDH18A1 | 0.39 | FG.Fibro.IFITM1.ADAMDEC1 | ASPN | 2.32 |
| FG.B/DZ.TOP2A.UBE2C | SIVA1 | 0.71 | FG.EC.GSTA2.AADAC | THOP1 | 0.39 | FG.Fibro.IFITM1.ADAMDEC1 | SERPING1 | 2.17 |
| FG.B/DZ.TOP2A.UBE2C | KLHL6 | 0.71 | FG.EC.GSTA2.AADAC | ZNF664 | 0.38 | FG.Fibro.IFITM1.ADAMDEC1 | ADH1B | 2.11 |
| FG.B/DZ.TOP2A.UBE2C | TIFA | 0.71 | FG.EC.GSTA2.AADAC | PDLIM5 | 0.38 | FG.Fibro.IFITM1.ADAMDEC1 | EDIL3 | 1.96 |
| FG.B/DZ.TOP2A.UBE2C | SRSF2 | 0.71 | FG.EC.GSTA2.AADAC | MAF | 0.38 | FG.Fibro.IFITM1.ADAMDEC1 | PTN | 1.95 |
| FG.B/DZ.TOP2A.UBE2C | COX17 | 0.71 | FG.EC.GSTA2.AADAC | SHROOM3 | 0.38 | FG.Fibro.IFITM1.ADAMDEC1 | ANGPTL1 | 1.92 |
| FG.B/DZ.TOP2A.UBE2C | SHCBP1 | 0.70 | FG.EC.GSTA2.AADAC | HEBP2 | 0.38 | FG.Fibro.IFITM1.ADAMDEC1 | BMP5 | 1.77 |
| FG.B/DZ.TOP2A.UBE2C | DEF8 | 0.70 | FG.EC.GSTA2.AADAC | TRIB1 | 0.38 | FG.Fibro.IFITM1.ADAMDEC1 | HAPLN1 | 1.74 |
| FG.B/DZ.TOP2A.UBE2C | HJURP | 0.70 | FG.EC.GSTA2.AADAC | FAM13A | 0.38 | FG.Fibro.IFITM1.ADAMDEC1 | SFTA1P | 1.53 |
| FG.B/DZ.TOP2A.UBE2C | PRDX6 | 0.70 | FG.EC.GSTA2.AADAC | ATP2B1 | 0.38 | FG.Fibro.IFITM1.ADAMDEC1 | SNAI2 | 1.62 |
| FG.B/DZ.TOP2A.UBE2C | UBE2N | 0.70 | FG.EC.GSTA2.AADAC | GALNT3 | 0.38 | FG.Fibro.MT-ND4L | CXCL14 | 3.14 |
| FG.B/DZ.TOP2A.UBE2C | PGAM1 | 0.70 | FG.EC.GSTA2.AADAC | PLD1 | 0.38 | FG.Fibro.MT-ND4L | ADAMDEC1 | 2.97 |
| FG.B/DZ.TOP2A.UBE2C | RP11-620J15.3 | 0.70 | FG.EC.GSTA2.AADAC | CEP70 | 0.38 | FG.Fibro.MT-ND4L | COL3A1 | 2.95 |
| FG.B/DZ.TOP2A.UBE2C | ILF2 | 0.70 | FG.EC.GSTA2.AADAC | CAPNS1 | 0.38 | FG.Fibro.MT-ND4L | CCL11 | 2.86 |
| FG.B/DZ.TOP2A.UBE2C | WDR76 | 0.70 | FG.EC.GSTA2.AADAC | COX14 | 0.38 | FG.Fibro.MT-ND4L | APOE | 2.84 |
| FG.B/DZ.TOP2A.UBE2C | CAPZA1 | 0.70 | FG.EC.GSTA2.AADAC | VDR | 0.38 | FG.Fibro.MT-ND4L | LUM | 2.62 |
| FG.B/DZ.TOP2A.UBE2C | MZT1 | 0.70 | FG.EC.GSTA2.AADAC | SMPD3 | 0.38 | FG.Fibro.MT-ND4L | DCN | 2.61 |
| FG.B/DZ.TOP2A.UBE2C | HAUS8 | 0.70 | FG.EC.GSTA2.AADAC | C11orf54 | 0.38 | FG.Fibro.MT-ND4L | CFD | 2.57 |
| FG.B/DZ.TOP2A.UBE2C | TKT | 0.70 | FG.EC.GSTA2.AADAC | KLC4 | 0.38 | FG.Fibro.MT-ND4L | COL1A2 | 2.50 |
| FG.B/DZ.TOP2A.UBE2C | ATAD2 | 0.69 | FG.EC.GSTA2.AADAC | HOOK2 | 0.38 | FG.Fibro.MT-ND4L | RARRES2 | 2.44 |
| FG.B/DZ.TOP2A.UBE2C | ATP5J2 | 0.69 | FG.EC.GSTA2.AADAC | PHPT1 | 0.38 | FG.Fibro.MT-ND4L | CALD1 | 2.43 |
| FG.B/DZ.TOP2A.UBE2C | MIS18BP1 | 0.69 | FG.EC.GSTA2.AADAC | SEC31A | 0.38 | FG.Fibro.MT-ND4L | CFH | 2.36 |
| FG.B/DZ.TOP2A.UBE2C | RBX1 | 0.69 | FG.EC.GSTA2.AADAC | PFKL | 0.38 | FG.Fibro.MT-ND4L | EDIL3 | 2.24 |
| FG.B/DZ.TOP2A.UBE2C | SNRPF | 0.69 | FG.EC.GSTA2.AADAC | PKP3 | 0.38 | FG.Fibro.MT-ND4L | FBLN1 | 2.19 |
| FG.B/DZ.TOP2A.UBE2C | COTL1 | 0.69 | FG.EC.GSTA2.AADAC | ANO10 | 0.38 | FG.Fibro.MT-ND4L | C1S | 2.26 |
| FG.B/DZ.TOP2A.UBE2C | KIF20B | 0.69 | FG.EC.GSTA2.AADAC | HIBCH | 0.38 | FG.Fibro.MT-ND4L | MFAP4 | 2.11 |
| FG.B/DZ.TOP2A.UBE2C | RFC3 | 0.69 | FG.EC.GSTA2.AADAC | ANO6 | 0.38 | FG.Fibro.MT-ND4L | C1R | 2.29 |
| FG.B/DZ.TOP2A.UBE2C | NASP | 0.69 | FG.EC.GSTA2.AADAC | SLC26A2 | 0.38 | FG.Fibro.RPS10.RPS12 | CXCL14 | 3.52 |
| FG.B/DZ.TOP2A.UBE2C | NAP1L1 | 0.69 | FG.EC.GSTA2.AADAC | NAMPT | 0.37 | FG.Fibro.RPS10.RPS12 | APOE | 2.96 |
| FG.B/DZ.TOP2A.UBE2C | MS4A1 | 0.69 | FG.EC.GSTA2.AADAC | HOOK1 | 0.37 | FG.Fibro.RPS10.RPS12 | CCL8 | 2.81 |
| FG.B/DZ.TOP2A.UBE2C | CDCA4 | 0.69 | FG.EC.GSTA2.AADAC | GPA33 | 0.37 | FG.Fibro.RPS10.RPS12 | CCL11 | 2.76 |
| FG.B/DZ.TOP2A.UBE2C | GSTP1 | 0.68 | FG.EC.GSTA2.AADAC | METTL7B | 0.37 | FG.Fibro.RPS10.RPS12 | CFD | 2.74 |
| FG.B/DZ.TOP2A.UBE2C | CCNI | 0.68 | FG.EC.GSTA2.AADAC | ENTPD5 | 0.37 | FG.Fibro.RPS10.RPS12 | PTGDS | 2.68 |
| FG.B/DZ.TOP2A.UBE2C | PIM1 | 0.68 | FG.EC.GSTA2.AADAC | IYD | 0.37 | FG.Fibro.RPS10.RPS12 | CALD1 | 2.63 |
| FG.B/DZ.TOP2A.UBE2C | RBMX | 0.68 | FG.EC.GSTA2.AADAC | AKAP1 | 0.37 | FG.Fibro.RPS10.RPS12 | COL3A1 | 2.46 |
| FG.B/DZ.TOP2A.UBE2C | BANF1 | 0.68 | FG.EC.GSTA2.AADAC | LAPTM4B | 0.37 | FG.Fibro.RPS10.RPS12 | ADAMDEC1 | 2.41 |
| FG.B/DZ.TOP2A.UBE2C | ENO1 | 0.68 | FG.EC.GSTA2.AADAC | ACKR4 | 0.37 | FG.Fibro.RPS10.RPS12 | COL1A2 | 2.39 |
| FG.B/DZ.TOP2A.UBE2C | LDHB | 0.67 | FG.EC.GSTA2.AADAC | ACY3 | 0.37 | FG.Fibro.RPS10.RPS12 | LUM | 2.39 |
| FG.B/DZ.TOP2A.UBE2C | SSRP1 | 0.67 | FG.EC.GSTA2.AADAC | ARHGEF35 | 0.37 | FG.Fibro.RPS10.RPS12 | RARRES2 | 2.34 |
| FG.B/DZ.TOP2A.UBE2C | ATP5G3 | 0.67 | FG.EC.GSTA2.AADAC | FDX1 | 0.37 | FG.Fibro.RPS10.RPS12 | A2M | 2.27 |
| FG.B/DZ.TOP2A.UBE2C | PPP4C | 0.67 | FG.EC.GSTA2.AADAC | IDH3G | 0.37 | FG.Fibro.RPS10.RPS12 | CCL2 | 2.23 |
| FG.B/DZ.TOP2A.UBE2C | VNN2 | 0.67 | FG.EC.GSTA2.AADAC | LASP1 | 0.37 | FG.Fibro.RPS10.RPS12 | DCN | 2.21 |
| FG.B/DZ.TOP2A.UBE2C | AC023590.1 | 0.67 | FG.EC.GSTA2.AADAC | PCYT1A | 0.37 | FG.Fibro.RPS10.RPS12 | ABCA8 | 2.16 |
| FG.B/DZ.TOP2A.UBE2C | LSM3 | 0.67 | FG.EC.GSTA2.AADAC | CLTA | 0.37 | FG.Fibro.RPS10.RPS12 | TAGLN | 2.11 |
| FG.B/DZ.TOP2A.UBE2C | UBE2E1 | 0.66 | FG.EC.GSTA2.AADAC | MEP1B | 0.37 | FG.Fibro.RPS10.RPS12 | RBP1 | 2.08 |
| FG.B/DZ.TOP2A.UBE2C | ACADM | 0.66 | FG.EC.GSTA2.AADAC | SURF4 | 0.37 | FG.Fibro.RPS10.RPS12 | C1S | 2.06 |
| FG.B/DZ.TOP2A.UBE2C | TBCA | 0.66 | FG.EC.GSTA2.AADAC | RHOB | 0.37 | FG.Fibro.RPS10.RPS12 | FBLN1 | 2.06 |
| FG.B/DZ.TOP2A.UBE2C | YWHAB | 0.66 | FG.EC.GSTA2.AADAC | MACC1 | 0.37 | FG.Fibro.RPS10.RPS12 | S100A13 | 2.05 |
| FG.B/DZ.TOP2A.UBE2C | PPP2CA | 0.66 | FG.EC.GSTA2.AADAC | PRELID3B | 0.37 | FG.Fibro.RPS10.RPS12 | PPP1R14A | 2.04 |
| FG.B/DZ.TOP2A.UBE2C | FCRLA | 0.66 | FG.EC.GSTA2.AADAC | CORO2A | 0.37 | FG.Fibro.RPS10.RPS12 | PTN | 2.04 |
| FG.B/DZ.TOP2A.UBE2C | CLTA | 0.66 | FG.EC.GSTA2.AADAC | ARPC1A | 0.36 | FG.Fibro.RPS10.RPS12 | MFAP4 | 2.00 |
| FG.B/DZ.TOP2A.UBE2C | PSMB2 | 0.66 | FG.EC.GSTA2.AADAC | NUDT9 | 0.36 | FG.Fibro.RPS10.RPS12 | EDIL3 | 1.99 |
| FG.B/DZ.TOP2A.UBE2C | CCT5 | 0.66 | FG.EC.GSTA2.AADAC | IFRD1 | 0.36 | FG.Fibro.RPS10.RPS12 | SPARCL1 | 1.94 |
| FG.B/DZ.TOP2A.UBE2C | ZCCHC7 | 0.66 | FG.EC.GSTA2.AADAC | APOL6 | 0.36 | FG.Fibro.RPS10.RPS12 | CFH | 1.90 |
| FG.B/DZ.TOP2A.UBE2C | TOP1 | 0.65 | FG.EC.GSTA2.AADAC | TMEM261 | 0.36 | FG.Fibro.RPS10.RPS12 | IGFBP7 | 1.84 |
| FG.B/DZ.TOP2A.UBE2C | MRPS6 | 0.65 | FG.EC.GSTA2.AADAC | DST | 0.36 | FG.Fibro.RPS10.RPS12 | MYL9 | 1.82 |
| FG.B/DZ.TOP2A.UBE2C | CCDC69 | 0.65 | FG.EC.GSTA2.AADAC | GATA4 | 0.36 | FG.Fibro.RPS10.RPS12 | PTCH1 | 1.79 |
| FG.B/DZ.TOP2A.UBE2C | HNRNPR | 0.65 | FG.EC.GSTA2.AADAC | PPP1R14B | 0.36 | FG.Fibro.RPS10.RPS12 | PLPP3 | 1.79 |
| FG.B/DZ.TOP2A.UBE2C | RFC4 | 0.65 | FG.EC.GSTA2.AADAC | ATRN | 0.36 | FG.Fibro.RPS10.RPS12 | TCF21 | 1.77 |
| FG.B/DZ.TOP2A.UBE2C | C14orf166 | 0.65 | FG.EC.GSTA2.AADAC | ESD | 0.36 | FG.Fibro.RPS10.RPS12 | TPM2 | 1.75 |
| FG.B/DZ.TOP2A.UBE2C | UBE2I | 0.65 | FG.EC.GSTA2.AADAC | FLRT3 | 0.36 | FG.Fibro.RPS10.RPS12 | ACTA2 | 1.75 |
| FG.B/DZ.TOP2A.UBE2C | SEC14L1 | 0.65 | FG.EC.GSTA2.AADAC | INPP1 | 0.36 | FG.Fibro.RPS10.RPS12 | COL6A2 | 1.74 |
| FG.B/DZ.TOP2A.UBE2C | DCAF12 | 0.64 | FG.EC.GSTA2.AADAC | PLIN3 | 0.36 | FG.Fibro.RPS10.RPS12 | C1R | 1.73 |
| FG.B/DZ.TOP2A.UBE2C | SUSD3 | 0.64 | FG.EC.GSTA2.AADAC | F11R | 0.36 | FG.Fibro.RPS10.RPS12 | MMP2 | 1.70 |
| FG.B/DZ.TOP2A.UBE2C | MCM4 | 0.64 | FG.EC.GSTA2.AADAC | FUCA1 | 0.36 | FG.Fibro.RPS10.RPS12 | LINC01082 | 1.65 |
| FG.B/DZ.TOP2A.UBE2C | NONO | 0.64 | FG.EC.GSTA2.AADAC | FZD5 | 0.36 | FG.Fibro.RPS10.RPS12 | PLEKHH2 | 1.57 |
| FG.B/DZ.TOP2A.UBE2C | SERBP1 | 0.64 | FG.EC.GSTA2.AADAC | NFIB | 0.36 | FG.Fibro.RPS10.RPS12 | WFDC1 | 1.54 |
| FG.B/DZ.TOP2A.UBE2C | HNRNPAB | 0.64 | FG.EC.GSTA2.AADAC | CNIH4 | 0.36 | FG.Fibro.RPS10.RPS12 | SPARC | 1.52 |
| FG.B/DZ.TOP2A.UBE2C | YEATS4 | 0.64 | FG.EC.GSTA2.AADAC | RNF128 | 0.36 | FG.Fibro.RPS10.RPS12 | NID1 | 1.51 |
| FG.B/DZ.TOP2A.UBE2C | E2F5 | 0.64 | FG.EC.GSTA2.AADAC | TMEM92 | 0.36 | FG.Fibro.RPS10.RPS12 | ITIH5 | 1.44 |
| FG.B/DZ.TOP2A.UBE2C | HNRNPK | 0.64 | FG.EC.GSTA2.AADAC | NCOA7 | 0.36 | FG.Fibro.RPS10.RPS12 | PDGFRA | 1.32 |
| FG.B/DZ.TOP2A.UBE2C | TPM4 | 0.64 | FG.EC.GSTA2.AADAC | BTD | 0.35 | FG.Fibro.RPS10.RPS12 | NOVA1 | 1.31 |
| FG.B/DZ.TOP2A.UBE2C | LPP | 0.64 | FG.EC.GSTA2.AADAC | NDFIP2 | 0.35 | FG.Fibro.RPS10.RPS12 | PCDH7 | 1.29 |
| FG.B/DZ.TOP2A.UBE2C | PSMA2 | 0.64 | FG.EC.GSTA2.AADAC | MRPL55 | 0.35 | FG.Fibro.TNFSF11.CD24 | C3 | 2.71 |
| FG.B/DZ.TOP2A.UBE2C | PPM1G | 0.64 | FG.EC.GSTA2.AADAC | MYRFL | 0.35 | FG.Fibro.TNFSF11.CD24 | SOD3 | 2.55 |
| FG.B/DZ.TOP2A.UBE2C | UHRF1 | 0.64 | FG.EC.GSTA2.AADAC | GJB1 | 0.35 | FG.Fibro.TNFSF11.CD24 | TNFSF11 | 1.87 |
| FG.B/DZ.TOP2A.UBE2C | SAE1 | 0.63 | FG.EC.GSTA2.AADAC | VPS28 | 0.35 | FG.Glial.BCYRN1.SPARC | PLP1 | 3.86 |
| FG.B/DZ.TOP2A.UBE2C | CNTRL | 0.63 | FG.EC.GSTA2.AADAC | CLINT1 | 0.35 | FG.Glial.BCYRN1.SPARC | SPP1 | 3.35 |
| FG.B/DZ.TOP2A.UBE2C | RUVBL1 | 0.63 | FG.EC.GSTA2.AADAC | AFG3L2 | 0.35 | FG.Glial.BCYRN1.SPARC | CRYAB | 3.23 |
| FG.B/DZ.TOP2A.UBE2C | GNG5 | 0.63 | FG.EC.GSTA2.AADAC | FNBP1L | 0.35 | FG.Glial.BCYRN1.SPARC | LGI4 | 2.94 |
| FG.B/DZ.TOP2A.UBE2C | FAM107B | 0.63 | FG.EC.GSTA2.AADAC | AQP11 | 0.35 | FG.Glial.BCYRN1.SPARC | MPZ | 2.73 |
| FG.B/DZ.TOP2A.UBE2C | POMP | 0.63 | FG.EC.GSTA2.AADAC | NAT2 | 0.35 | FG.Glial.BCYRN1.SPARC | CDH19 | 2.68 |
| FG.B/DZ.TOP2A.UBE2C | TRA2B | 0.63 | FG.EC.GSTA2.AADAC | KDELR1 | 0.35 | FG.Glial.BCYRN1.SPARC | GFRA3 | 2.62 |
| FG.B/DZ.TOP2A.UBE2C | DYNLL1 | 0.63 | FG.EC.GSTA2.AADAC | PHB | 0.35 | FG.Glial.BCYRN1.SPARC | S100B | 2.61 |
| FG.B/DZ.TOP2A.UBE2C | VPREB3 | 0.63 | FG.EC.GSTA2.AADAC | RP11-30P6.6 | 0.35 | FG.Glial.BCYRN1.SPARC | SCN7A | 2.59 |
| FG.B/DZ.TOP2A.UBE2C | KIF2C | 0.63 | FG.EC.GSTA2.AADAC | CMAS | 0.35 | FG.Glial.BCYRN1.SPARC | NRXN1 | 2.36 |
| FG.B/DZ.TOP2A.UBE2C | BCL2L12 | 0.63 | FG.EC.GSTA2.AADAC | PDXDC1 | 0.35 | FG.Glial.BCYRN1.SPARC | BCYRN1 | 2.20 |
| FG.B/DZ.TOP2A.UBE2C | MXD3 | 0.63 | FG.EC.GSTA2.AADAC | MYO7B | 0.35 | FG.Glial.BCYRN1.SPARC | ST6GALNAC2 | 2.02 |
| FG.B/DZ.TOP2A.UBE2C | CENPK | 0.63 | FG.EC.GSTA2.AADAC | SEC23A | 0.35 | FG.Glial.BCYRN1.SPARC | PCSK2 | 1.89 |
| FG.B/DZ.TOP2A.UBE2C | LYPLA1 | 0.63 | FG.EC.GSTA2.AADAC | CRAT | 0.35 | FG.Glial.BCYRN1.SPARC | MYOT | 1.83 |
| FG.B/DZ.TOP2A.UBE2C | SLC25A3 | 0.62 | FG.EC.GSTA2.AADAC | TMEM14A | 0.35 | FG.Glial.BCYRN1.SPARC | XKR4 | 1.79 |
| FG.B/DZ.TOP2A.UBE2C | MSH6 | 0.62 | FG.EC.GSTA2.AADAC | TRIM15 | 0.34 | FG.Glial.BCYRN1.SPARC | NGFR | 1.78 |
| FG.B/DZ.TOP2A.UBE2C | CASC5 | 0.62 | FG.EC.GSTA2.AADAC | OVOL1 | 0.34 | FG.Glial.BCYRN1.SPARC | NTM | 1.74 |
| FG.B/DZ.TOP2A.UBE2C | CDCA5 | 0.62 | FG.EC.GSTA2.AADAC | ARHGAP5 | 0.34 | FG.Glial.BCYRN1.SPARC | PTPRZ1 | 1.73 |
| FG.B/DZ.TOP2A.UBE2C | SMC3 | 0.62 | FG.EC.GSTA2.AADAC | ACSS2 | 0.34 | FG.Glial.BCYRN1.SPARC | LRRTM1 | 1.71 |
| FG.B/DZ.TOP2A.UBE2C | NUDT1 | 0.62 | FG.EC.GSTA2.AADAC | MLXIPL | 0.34 | FG.Glial.BCYRN1.SPARC | NRN1 | 2.13 |
| FG.B/DZ.TOP2A.UBE2C | DAZAP1 | 0.62 | FG.EC.GSTA2.AADAC | TJP3 | 0.34 | FG.Glial.HAND2.MT-ND4L | SPP1 | 3.72 |
| FG.B/DZ.TOP2A.UBE2C | ATP5J | 0.62 | FG.EC.GSTA2.AADAC | FH | 0.34 | FG.Glial.HAND2.MT-ND4L | CRYAB | 3.70 |
| FG.B/DZ.TOP2A.UBE2C | LSM14A | 0.62 | FG.EC.GSTA2.AADAC | HKDC1 | 0.34 | FG.Glial.HAND2.MT-ND4L | PLP1 | 3.29 |
| FG.B/DZ.TOP2A.UBE2C | BUB1 | 0.62 | FG.EC.GSTA2.AADAC | MEIS2 | 0.34 | FG.Glial.HAND2.MT-ND4L | FXYD1 | 3.23 |
| FG.B/DZ.TOP2A.UBE2C | HNRNPH3 | 0.61 | FG.EC.GSTA2.AADAC | NET1 | 0.34 | FG.Glial.HAND2.MT-ND4L | PMP22 | 2.94 |
| FG.B/DZ.TOP2A.UBE2C | PXK | 0.61 | FG.EC.GSTA2.AADAC | MIDN | 0.34 | FG.Glial.HAND2.MT-ND4L | SCN7A | 2.90 |
| FG.B/DZ.TOP2A.UBE2C | SPIB | 0.61 | FG.EC.GSTA2.AADAC | MYO15B | 0.34 | FG.Glial.HAND2.MT-ND4L | GPM6B | 2.88 |
| FG.B/DZ.TOP2A.UBE2C | MZT2A | 0.61 | FG.EC.GSTA2.AADAC | ABCG5 | 0.34 | FG.Glial.HAND2.MT-ND4L | SPARC | 2.87 |
| FG.B/DZ.TOP2A.UBE2C | NCF1 | 0.61 | FG.EC.GSTA2.AADAC | ABCC3 | 0.34 | FG.Glial.HAND2.MT-ND4L | S100B | 2.83 |
| FG.B/DZ.TOP2A.UBE2C | TUBA1C | 0.61 | FG.EC.GSTA2.AADAC | SCIN | 0.34 | FG.Glial.HAND2.MT-ND4L | LGI4 | 2.68 |
| FG.B/DZ.TOP2A.UBE2C | CCDC109B | 0.61 | FG.EC.GSTA2.AADAC | TMEM126A | 0.34 | FG.Glial.HAND2.MT-ND4L | TIMP3 | 2.63 |
| FG.B/DZ.TOP2A.UBE2C | TPGS2 | 0.61 | FG.EC.GSTA2.AADAC | FBLIM1 | 0.34 | FG.Glial.HAND2.MT-ND4L | CDH19 | 2.43 |
| FG.B/DZ.TOP2A.UBE2C | FAM111B | 0.61 | FG.EC.GSTA2.AADAC | MUC17 | 0.34 | FG.Glial.HAND2.MT-ND4L | GFRA3 | 2.38 |
| FG.B/DZ.TOP2A.UBE2C | PRPSAP2 | 0.60 | FG.EC.GSTA2.AADAC | PGM1 | 0.34 | FG.Glial.HAND2.MT-ND4L | CYR61 | 2.34 |
| FG.B/DZ.TOP2A.UBE2C | RFC2 | 0.60 | FG.EC.GSTA2.AADAC | OXA1L | 0.34 | FG.Glial. HAND2.MT-ND4L | NRXN1 | 2.32 |
| FG.B/DZ.TOP2A.UBE2C | ARHGDIB | 0.60 | FG.EC.GSTA2.AADAC | FMO5 | 0.34 | FG.Glial.HAND2.MT-ND4L | MYOT | 2.20 |
| FG.B/DZ.TOP2A.UBE2C | CDCA7L | 0.60 | FG.EC.GSTA2.AADAC | ARL5B | 0.34 | FG.Glial.HAND2.MT-ND4L | MPZ | 1.91 |
| FG.B/DZ.TOP2A.UBE2C | SYNE2 | 0.60 | FG.EC.GSTA2.AADAC | VDAC3 | 0.34 | FG.Glial.HAND2.MT-ND4L | NRN1 | 1.73 |
| FG.B/DZ.TOP2A.UBE2C | ORC6 | 0.60 | FG.EC.GSTA2.AADAC | CHMP4C | 0.34 | FG.Glial.HAND2.MT-ND4L | PTPRZ1 | 1.72 |
| FG.B/DZ.TOP2A.UBE2C | ATP5F1 | 0.60 | FG.EC.GSTA2.AADAC | GALK1 | 0.34 | FG.Glial.HAND2.MT-ND4L | HAND2 | 1.63 |
| FG.B/DZ.TOP2A.UBE2C | SUGCT | 0.60 | FG.EC.GSTA2.AADAC | CHN2 | 0.34 | FG.Glial.HAND2.MT-ND4L | XKR4 | 1.62 |
| FG.B/DZ.TOP2A.UBE2C | UBE2L3 | 0.60 | FG.EC.GSTA2.AADAC | SURF1 | 0.34 | FG.Glial.HAND2.MT-ND4L | NTM | 1.54 |
| FG.B/DZ.TOP2A.UBE2C | HAUS1 | 0.60 | FG.EC.GSTA2.AADAC | LACTB2 | 0.34 | FG.Glial.HAND2.MT-ND4L | COL21A1 | 1.48 |
| FG.B/DZ.TOP2A.UBE2C | TRAPPC1 | 0.60 | FG.EC.GSTA2.AADAC | TOMM20 | 0.34 | FG.Glial.HAND2.MT-ND4L | TSPAN11 | 1.35 |
| FG.B/DZ.TOP2A.UBE2C | SNRPC | 0.60 | FG.EC.GSTA2.AADAC | MRPL14 | 0.34 | FG.Glial.HAND2.MT-ND4L | L1CAM | 1.34 |
| FG.B/DZ.TOP2A.UBE2C | EIF2S2 | 0.59 | FG.EC.GSTA2.AADAC | CCL28 | 0.34 | FG.Glial.HAND2.MT-ND4L | FOXD3-AS1 | 1.29 |
| FG.B/DZ.TOP2A.UBE2C | RBM17 | 0.59 | FG.EC.GSTA2.AADAC | PHYH | 0.34 | FG.Glial.HAND2.MT-ND4L | LRRTM1 | 1.16 |
| FG.B/DZ.TOP2A.UBE2C | MTF2 | 0.59 | FG.EC.GSTA2.AADAC | NCKAP1 | 0.34 | FG.Glial.HAND2.MT-ND4L | ART3 | 1.15 |
| FG.B/DZ.TOP2A.UBE2C | SYPL1 | 0.59 | FG.EC.GSTA2.AADAC | MOB3B | 0.34 | FG.Glial.HSPA1B.SOCS3 | CYR61 | 4.14 |
| FG.B/DZ.TOP2A.UBE2C | RPRD1B | 0.59 | FG.EC.GSTA2.AADAC | GK5 | 0.34 | FG.Glial.HSPA1B.SOCS3 | SPARC | 3.84 |
| FG.B/DZ.TOP2A.UBE2C | NAA50 | 0.59 | FG.EC.GSTA2.AADAC | C1orf210 | 0.33 | FG.Glial.HSPA1B.SOCS3 | PLP1 | 3.82 |
| FG.B/DZ.TOP2A.UBE2C | SSBP2 | 0.59 | FG.EC.GSTA2.AADAC | RTCB | 0.33 | FG.Glial.HSPA1B.SOCS3 | CRYAB | 3.32 |
| FG.B/DZ.TOP2A.UBE2C | DEPDC1 | 0.59 | FG.EC.GSTA2.AADAC | RHOU | 0.33 | FG.Glial.HSPA1B.SOCS3 | SPP1 | 3.27 |
| FG.B/DZ.TOP2A.UBE2C | LSM10 | 0.59 | FG.EC.GSTA2.AADAC | SLC27A4 | 0.33 | FG.Glial.HSPA1B.SOCS3 | PMP22 | 3.25 |
| FG.B/DZ.TOP2A.UBE2C | SMC1A | 0.59 | FG.EC.GSTA2.AADAC | ABHD11-AS1 | 0.33 | FG.Glial.HSPA1B.SOCS3 | SEMA3B | 3.10 |
| FG.B/DZ.TOP2A.UBE2C | POU2F2 | 0.58 | FG.EC.GSTA2.AADAC | VPS13A | 0.33 | FG.Glial.HSPA1B.SOCS3 | LGI4 | 2.84 |
| FG.B/DZ.TOP2A.UBE2C | MTHFD2 | 0.58 | FG.EC.GSTA2.AADAC | CHMP4B | 0.33 | FG.Glial.HSPA1B.SOCS3 | MATN2 | 2.76 |
| FG.B/DZ.TOP2A.UBE2C | VPS29 | 0.58 | FG.EC.GSTA2.AADAC | H1F0 | 0.33 | FG.Glial.HSPA1B.SOCS3 | MPZ | 2.69 |
| FG.B/DZ.TOP2A.UBE2C | PSMB3 | 0.58 | FG.EC.GSTA2.AADAC | GADD45A | 0.33 | FG.Glial.HSPA1B.SOCS3 | CDH19 | 2.68 |
| FG.B/DZ.TOP2A.UBE2C | GTF2A2 | 0.58 | FG.EC.GSTA2.AADAC | TMCO1 | 0.33 | FG.Glial.HSPA1B.SOCS3 | GFRA3 | 2.68 |
| FG.B/DZ.TOP2A.UBE2C | EIF31 | 0.58 | FG.EC.GSTA2.AADAC | MRPL34 | 0.33 | FG.Glial.HSPA1B.SOCS3 | TIMP3 | 2.64 |
| FG.B/DZ.TOP2A.UBE2C | ACY3 | 0.58 | FG.EC.GSTA2.AADAC | TECR | 0.33 | FG.Glial.HSPA1B.SOCS3 | DKK3 | 2.50 |
| FG.B/DZ.TOP2A.UBE2C | FAM76B | 0.58 | FG.EC.GSTA2.AADAC | LAD1 | 0.33 | FG.Glial.HSPA1B.SOCS3 | S100B | 2.40 |
| FG.B/DZ.TOP2A.UBE2C | GLRX3 | 0.58 | FG.EC.GSTA2.AADAC | MRPS35 | 0.33 | FG.Glial.HSPA1B.SOCS3 | SCN7A | 2.34 |
| FG.B/DZ.TOP2A.UBE2C | SF3B6 | 0.58 | FG.EC.GSTA2.AADAC | CDKN2B | 0.33 | FG.Glial.HSPA1B.SOCS3 | FSTL3 | 2.30 |
| FG.B/DZ.TOP2A.UBE2C | BRK1 | 0.58 | FG.EC.GSTA2.AADAC | RP11-49111.1 | 0.33 | FG.Glial.HSPA1B.SOCS3 | MOXD1 | 2.10 |
| FG.B/DZ.TOP2A.UBE2C | STRBP | 0.58 | FG.EC.GSTA2.AADAC | MRPL35 | 0.33 | FG.Glial.HSPA1B.SOCS3 | NRXN1 | 2.09 |
| FG.B/DZ.TOP2A.UBE2C | CENPN | 0.58 | FG.EC.GSTA2.AADAC | MAN2A1 | 0.33 | FG.Glial.HSPA1B.SOCS3 | VWA1 | 2.06 |
| FG.B/DZ.TOP2A.UBE2C | ARPC1B | 0.57 | FG.EC.GSTA2.AADAC | HAAO | 0.33 | FG.Glial.HSPA1B.SOCS3 | MAP1B | 2.04 |
| FG.B/DZ.TOP2A.UBE2C | HSPB11 | 0.57 | FG.EC.GSTA2.AADAC | SLC25A24 | 0.33 | FG.Glial.HSPA1B.SOCS3 | NNMT | 1.90 |
| FG.B/DZ.TOP2A.UBE2C | CDK2AP2 | 0.57 | FG.EC.GSTA2.AADAC | PVRL3 | 0.33 | FG.Glial.HSPA1B.SOCS3 | PHLDA3 | 1.89 |
| FG.B/DZ.TOP2A.UBE2C | CD22 | 0.57 | FG.EC.GSTA2.AADAC | LINC00675 | 0.33 | FG.Glial.HSPA1B.SOCS3 | PLEKHB1 | 1.86 |
| FG.B/DZ.TOP2A.UBE2C | CDCA2 | 0.57 | FG.EC.GSTA2.AADAC | IDH3B | 0.33 | FG.Glial.HSPA1B.SOCS3 | XKR4 | 1.85 |
| FG.B/DZ.TOP2A.UBE2C | RALY | 0.57 | FG.EC.GSTA2.AADAC | C1orf21 | 0.33 | FG.Glial.HSPA1B.SOCS3 | NGFR | 1.83 |
| FG.B/DZ.TOP2A.UBE2C | UBE2A | 0.57 | FG.EC.GSTA2.AADAC | ZBTB7B | 0.33 | FG.Glial.HSPA1B.SOCS3 | NTM | 1.82 |
| FG.B/DZ.TOP2A.UBE2C | PHF19 | 0.57 | FG.EC.GSTA2.AADAC | RILP | 0.33 | FG.Glial.HSPA1B.SOCS3 | NRN1 | 1.82 |
| FG.B/DZ.TOP2A.UBE2C | CACYBP | 0.57 | FG.EC.GSTA2.AADAC | TMEM125 | 0.33 | FG.Glial.HSPA1B.SOCS3 | LRRTM1 | 1.82 |
| FG.B/DZ.TOP2A.UBE2C | TALDO1 | 0.57 | FG.EC.GSTA2.AADAC | RER1 | 0.33 | FG.Glial.HSPA1B.SOCS3 | SLC22A17 | 1.81 |
| FG.B/DZ.TOP2A.UBE2C | SNRPD3 | 0.57 | FG.EC.GSTA2.AADAC | QTRT1 | 0.33 | FG.Glial.HSPA1B.SOCS3 | TSPAN11 | 1.69 |
| FG.B/DZ.TOP2A.UBE2C | KIF11 | 0.57 | FG.EC.GSTA2.AADAC | ASAH2 | 0.33 | FG.Glial.HSPA1B.SOCS3 | MYOT | 1.69 |
| FG.B/DZ.TOP2A.UBE2C | ANP32A | 0.57 | FG.EC.GSTA2.AADAC | HEPH | 0.33 | FG.Glial.HSPA1B.SOCS3 | ST6GALNAC2 | 1.67 |
| FG.B/DZ.TOP2A.UBE2C | RRAS2 | 0.57 | FG.EC.GSTA2.AADAC | TMEM208 | 0.33 | FG.Glial.HSPA1B.SOCS3 | BCYRN1 | 1.60 |
| FG.B/DZ.TOP2A.UBE2C | WDR66 | 0.57 | FG.EC.GSTA2.AADAC | CROT | 0.32 | FG.Glial.HSPA1B.SOCS3 | SERPINA5 | 1.58 |
| FG.B/DZ.TOP2A.UBE2C | HMGN3 | 0.56 | FG.EC.GSTA2.AADAC | PTPRK | 0.32 | FG.Glial.HSPA1B.SOCS3 | PCSK2 | 1.56 |
| FG.B/DZ.TOP2A.UBE2C | SH3KBP1 | 0.56 | FG.EC.GSTA2.AADAC | MKKS | 0.32 | FG.Glial.HSPA1B.SOCS3 | CADM3 | 1.45 |
| FG.B/DZ.TOP2A.UBE2C | SPATS2 | 0.56 | FG.EC.GSTA2.AADAC | TMEM14C | 0.32 | FG.Glial.HSPA1B.SOCS3 | SNCA | 1.44 |
| FG.B/DZ.TOP2A.UBE2C | MYBL1 | 0.56 | FG.EC.GSTA2.AADAC | LPP | 0.32 | FG.Glial.HSPA1B.SOCS3 | COL8A1 | 1.44 |
| FG.B/DZ.TOP2A.UBE2C | LAPTM5 | 0.56 | FG.EC.GSTA2.AADAC | TMEM37 | 0.32 | FG.Glial.HSPA1B.SOCS3 | ANK2 | 1.43 |
| FG.B/DZ.TOP2A.UBE2C | POLR3K | 0.56 | FG.EC.GSTA2.AADAC | KIAA2013 | 0.32 | FG.Glial.HSPA1B.SOCS3 | SOX2 | 1.40 |
| FG.B/DZ.TOP2A.UBE2C | RNASEH2A | 0.56 | FG.EC.GSTA2.AADAC | TFPI | 0.32 | FG.Glial.HSPA1B.SOCS3 | RXRG | 1.37 |
| FG.B/DZ.TOP2A.UBE2C | BRCA1 | 0.56 | FG.EC.GSTA2.AADAC | STAU1 | 0.32 | FG.Glial.HSPA1B.SOCS3 | PTPRZ1 | 1.35 |
| FG.B/DZ.TOP2A.UBE2C | SELT | 0.56 | FG.EC.GSTA2.AADAC | NR1D1 | 0.32 | FG.Glial.HSPA1B.SOCS3 | CADM2 | 1.34 |
| FG.B/DZ.TOP2A.UBE2C | CCT2 | 0.56 | FG.EC.GSTA2.AADAC | TMED4 | 0.32 | FG.Glial.HSPA1B.SOCS3 | ANGPTL7 | 1.32 |
| FG.B/DZ.TOP2A.UBE2C | BLOC1S6 | 0.56 | FG.EC.GSTA2.AADAC | RAPGEFL1 | 0.32 | FG.Glial.HSPA1B.SOCS3 | COL28A1 | 1.31 |
| FG.B/DZ.TOP2A.UBE2C | BTF3 | 0.56 | FG.EC.GSTA2.AADAC | GCNT3 | 0.32 | FG.Glial.HSPA1B.SOCS3 | PMP2 | 1.31 |
| FG.B/DZ.TOP2A.UBE2C | HNRNPA0 | 0.56 | FG.EC.GSTA2.AADAC | TRAF4 | 0.32 | FG.Glial.HSPA1B.SOCS3 | CNN1 | 1.26 |
| FG.B/DZ.TOP2A.UBE2C | IDH2 | 0.55 | FG.EC.GSTA2.AADAC | PRADC1 | 0.32 | FG.Glial.HSPA1B.SOCS3 | NRXN3 | 1.25 |
| FG.B/DZ.TOP2A.UBE2C | OGG1 | 0.55 | FG.EC.GSTA2.AADAC | SH3YL1 | 0.32 | FG.Glial.HSPA1B.SOCS3 | INSC | 1.23 |
| FG.B/DZ.TOP2A.UBE2C | STAG3 | 0.55 | FG.EC.GSTA2.AADAC | RP11-467L13.7 | 0.32 | FG.Glial.HSPA1B.SOCS3 | TTYH1 | 1.23 |
| FG.B/DZ.TOP2A.UBE2C | CDC25B | 0.55 | FG.EC.GSTA2.AADAC | RP11-380P13.1 | 0.32 | FG.Glial.HSPA1B.SOCS3 | COL21A1 | 1.22 |
| FG.B/DZ.TOP2A.UBE2C | PSMD14 | 0.55 | FG.EC.GSTA2.AADAC | NBL1 | 0.32 | FG.Glial.HSPA1B.SOCS3 | FBLN2 | 1.21 |
| FG.B/DZ.TOP2A.UBE2C | FKBP3 | 0.55 | FG.EC.GSTA2.AADAC | CDC42EP5 | 0.32 | FG.Glial.HSPA1B.SOCS3 | SBSPON | 1.21 |
| FG.B/DZ.TOP2A.UBE2C | CCT6A | 0.55 | FG.EC.GSTA2.AADAC | TMEM139 | 0.32 | FG.Glial.HSPA1B.SOCS3 | MGAT4C | 1.21 |
| FG.B/DZ.TOP2A.UBE2C | RNGTT | 0.55 | FG.EC.GSTA2.AADAC | C1orf106 | 0.32 | FG.Glial.HSPA1B.SOCS3 | L1CAM | 1.21 |
| FG.B/DZ.TOP2A.UBE2C | ITGB3BP | 0.55 | FG.EC.GSTA2.AADAC | PVRL2 | 0.32 | FG.Glial.HSPA1B.SOCS3 | HAND2 | 1.19 |
| FG.B/DZ.TOP2A.UBE2C | ARPC4 | 0.55 | FG.EC.GSTA2.AADAC | JUP | 0.32 | FG.Glial.HSPA1B.SOCS3 | SHC4 | 1.18 |
| FG.B/DZ.TOP2A.UBE2C | KIAA0226L | 0.54 | FG.EC.GSTA2.AADAC | TM9SF2 | 0.32 | FG.Glial.HSPA1B.SOCS3 | CDH2 | 1.07 |
| FG.B/DZ.TOP2A.UBE2C | SFPQ | 0.54 | FG.EC.GSTA2.AADAC | APOA1BP | 0.32 | FG.Glial.HSPA1B.SOCS3 | GPR17 | 1.01 |
| FG.B/DZ.TOP2A.UBE2C | NUGGC | 0.54 | FG.EC.GSTA2.AADAC | RNF5 | 0.31 | FG.Glial.HSPA1B.SOCS3 | S1PR3 | 0.99 |
| FG.B/DZ.TOP2A.UBE2C | SHFM1 | 0.54 | FG.EC.GSTA2.AADAC | NUDT8 | 0.31 | FG.Glial.HSPA1B.SOCS3 | WDR86 | 0.94 |
| FG.B/DZ.TOP2A.UBE2C | HIST1H1E | 0.54 | FG.EC.GSTA2.AADAC | MAP7 | 0.31 | FG.Glial.HSPA1B.SOCS3 | SOX10 | 0.93 |
| FG.B/DZ.TOP2A.UBE2C | LRR1 | 0.54 | FG.EC.GSTA2.AADAC | SLC51A | 0.31 | FG.Glial.HSPA1B.SOCS3 | NLGN4X | 0.90 |
| FG.B/DZ.TOP2A.UBE2C | HIST1H1B | 0.54 | FG.EC.GSTA2.AADAC | GBP3 | 0.31 | FG.Glial.HSPA1B.SOCS3 | EFS | 0.89 |
| FG.B/DZ.TOP2A.UBE2C | RP11-132N15.3 | 0.54 | FG.EC.GSTA2.AADAC | ASS1 | 0.31 | FG.Glial.HSPA1B.SOCS3 | ST6GALNAC5 | 0.87 |
| FG.B/DZ.TOP2A.UBE2C | KIF23 | 0.54 | FG.EC.GSTA2.AADAC | TMBIM1 | 0.31 | FG.Glial.HSPA1B.SOCS3 | WISP2 | 0.78 |
| FG.B/DZ.TOP2A.UBE2C | PTP4A2 | 0.54 | FG.EC.GSTA2.AADAC | HRASLS2 | 0.31 | FG.Glial.HSPA1B.SOCS3 | GPM6B | 3.00 |
| FG.B/DZ.TOP2A.UBE2C | DBF4 | 0.54 | FG.EC.GSTA2.AADAC | GFPT1 | 0.31 | FG.Glial.HSPA1B.SOCS3 | MXRA8 | 1.79 |
| FG.B/DZ.TOP2A.UBE2C | NDUFV2 | 0.54 | FG.EC.GSTA2.AADAC | GSDMD | 0.31 | FG.Glial.HSPA1B.SOCS3 | HSPA2 | 1.99 |
| FG.B/DZ.TOP2A.UBE2C | ESCO2 | 0.54 | FG.EC.GSTA2.AADAC | MRPL15 | 0.31 | FG.Glial.HSPA1B.SOCS3 | SORCS1 | 1.13 |
| FG.B/DZ.TOP2A.UBE2C | RPLP0 | 0.54 | FG.EC.GSTA2.AADAC | UGDH | 0.31 | FG.Glial.HSPA1B.SOCS3 | SHISA9 | 0.61 |
| FG.B/DZ.TOP2A.UBE2C | KNSTRN | 0.53 | FG.EC.GSTA2.AADAC | DHDH | 0.31 | FG.Glial.IER2.JUNB | CRYAB | 3.99 |
| FG.B/DZ.TOP2A.UBE2C | CLIC4 | 0.53 | FG.EC.GSTA2.AADAC | SAMM50 | 0.31 | FG.Glial.IER2.JUNB | CYR61 | 3.61 |
| FG.B/DZ.TOP2A.UBE2C | POLR2J | 0.53 | FG.EC.GSTA2.AADAC | TNFRSF12A | 0.31 | FG.Glial.IER2.JUNB | SPP1 | 3.52 |
| FG.B/DZ.TOP2A.UBE2C | RACGAP1 | 0.53 | FG.EC.GSTA2.AADAC | SCCPDH | 0.31 | FG.Glial.IER2.JUNB | CLU | 3.30 |
| FG.B/DZ.TOP2A.UBE2C | EIF4H | 0.53 | FG.EC.GSTA2.AADAC | AKAP9 | 0.31 | FG.Glial.IER2.JUNB | PLP1 | 3.28 |
| FG.B/DZ.TOP2A.UBE2C | MAP2K1 | 0.53 | FG.EC.GSTA2.AADAC | GRTP1 | 0.31 | FG.Glial.IER2.JUNB | FXYD1 | 3.14 |
| FG.B/DZ.TOP2A.UBE2C | THRAP3 | 0.53 | FG.EC.GSTA2.AADAC | ABHD14B | 0.31 | FG.Glial.IER2.JUNB | PMP22 | 3.08 |
| FG.B/DZ.TOP2A.UBE2C | CHRAC1 | 0.53 | FG.EC.GSTA2.AADAC | CD46 | 0.31 | FG.Glial.IER2.JUNB | S100B | 3.00 |
| FG.B/DZ.TOP2A.UBE2C | FKBP1A | 0.53 | FG.EC.GSTA2.AADAC | MTUS1 | 0.30 | FG.Glial.IER2.JUNB | LGI4 | 2.92 |
| FG.B/DZ.TOP2A.UBE2C | LAT2 | 0.53 | FG.EC.GSTA2.AADAC | LLGL2 | 0.30 | FG.Glial.IER2.JUNB | GPM6B | 2.90 |
| FG.B/DZ.TOP2A.UBE2C | CCNG2 | 0.53 | FG.EC.GSTA2.AADAC | IMMT | 0.30 | FG.Glial.IER2.JUNB | CD9 | 2.89 |
| FG.B/DZ.TOP2A.UBE2C | CD38 | 0.53 | FG.EC.GSTA2.AADAC | RNF7 | 0.30 | FG.Glial.IER2.JUNB | CCL2 | 2.89 |
| FG.B/DZ.TOP2A.UBE2C | LYL1 | 0.52 | FG.EC.GSTA2.AADAC | ZNF91 | 0.30 | FG.Glial.IER2.JUNB | ALDH1A1 | 2.63 |
| FG.B/DZ.TOP2A.UBE2C | LIG1 | 0.52 | FG.EC.GSTA2.AADAC | MARVELD2 | 0.30 | FG.Glial.IER2.JUNB | SPARC | 2.58 |
| FG.B/DZ.TOP2A.UBE2C | DCTN3 | 0.52 | FG.EC.GSTA2.AADAC | SLC25A36 | 0.30 | FG.Glial.IER2.JUNB | TIMP3 | 2.54 |
| FG.B/DZ.TOP2A.UBE2C | PHGDH | 0.52 | FG.EC.GSTA2.AADAC | PECR | 0.30 | FG.Glial.IER2.JUNB | C8orf4 | 2.43 |
| FG.B/DZ.TOP2A.UBE2C | KIAA0922 | 0.52 | FG.EC.GSTA2.AADAC | LSR | 0.30 | FG.Glial.IER2.JUNB | PRNP | 2.42 |
| FG.B/DZ.TOP2A.UBE2C | RAD51AP1 | 0.52 | FG.EC.GSTA2.AADAC | ID2 | 0.30 | FG.Glial.IER2.JUNB | SEMA3B | 2.36 |
| FG.B/DZ.TOP2A.UBE2C | RASSF6 | 0.52 | FG.EC.GSTA2.AADAC | CRB3 | 0.30 | FG.Glial.IER2.JUNB | GFRA3 | 2.32 |
| FG.B/DZ.TOP2A.UBE2C | PRKDC | 0.52 | FG.EC.GSTA2.AADAC | SMPDL3B | 0.30 | FG.Glial.IER2.JUNB | SCN7A | 2.30 |
| FG.B/DZ.TOP2A.UBE2C | MCTS1 | 0.52 | FG.EC.GSTA2.AADAC | MRPS36 | 0.30 | FG.Glial.IER2.JUNB | NRXN1 | 2.12 |
| FG.B/DZ.TOP2A.UBE2C | SNRPA1 | 0.52 | FG.EC.GSTA2.AADAC | EHHADH | 0.30 | FG.Glial.IER2.JUNB | MATN2 | 2.01 |
| FG.B/DZ.TOP2A.UBE2C | MDH1 | 0.52 | FG.EC.GSTA2.AADAC | ARL14 | 0.30 | FG.Glial.IER2.JUNB | SPARCL1 | 1.92 |
| FG.B/DZ.TOP2A.UBE2C | HPRT1 | 0.52 | FG.EC.GSTA2.AADAC | GCNT4 | 0.30 | FG.Glial.IER2.JUNB | CDH19 | 1.92 |
| FG.B/DZ.TOP2A.UBE2C | MCM6 | 0.51 | FG.EC.GSTA2.AADAC | TMEM106C | 0.30 | FG.Glial.IER2.JUNB | IGFBP7 | 1.88 |
| FG.B/DZ.TOP2A.UBE2C | CNN2 | 0.51 | FG.EC.GSTA2.AADAC | SH3D19 | 0.30 | FG.Glial.IER2.JUNB | MPZ | 1.85 |
| FG.B/DZ.TOP2A.UBE2C | RASGRP3 | 0.51 | FG.EC.GSTA2.AADAC | APEX1 | 0.30 | FG.Glial.IER2.JUNB | DKK3 | 1.83 |
| FG.B/DZ.TOP2A.UBE2C | RAD51 | 0.51 | FG.EC.GSTA2.AADAC | PDX1 | 0.30 | FG.Glial.IER2.JUNB | MYOT | 1.78 |
| FG.B/DZ.TOP2A.UBE2C | NDUFB3 | 0.51 | FG.EC.GSTA2.AADAC | PTPRH | 0.30 | FG.Glial.IER2.JUNB | SORBS2 | 1.77 |
| FG.B/DZ.TOP2A.UBE2C | SPI1 | 0.51 | FG.EC.GSTA2.AADAC | MSRA | 0.30 | FG.Glial.IER2.JUNB | PLEKHB1 | 1.61 |
| FG.B/DZ.TOP2A.UBE2C | SH2B2 | 0.51 | FG.EC.GSTA2.AADAC | TFDP2 | 0.30 | FG.Glial.IER2.JUNB | CAB39L | 1.60 |
| FG.B/DZ.TOP2A.UBE2C | ANKRD13A | 0.51 | FG.EC.GSTA2.AADAC | VSIG10 | 0.30 | FG.Glial.IER2.JUNB | NTM | 1.55 |
| FG.B/DZ.TOP2A.UBE2C | ARPC5L | 0.50 | FG.EC.GSTA2.AADAC | MST1 | 0.30 | FG.Glial.IER2.JUNB | FSTL3 | 1.52 |
| FG.B/DZ.TOP2A.UBE2C | NUTF2 | 0.50 | FG.EC.GSTA2.AADAC | GNB1 | 0.30 | FG.Glial.IER2.JUNB | SMIM5 | 1.46 |
| FG.B/DZ.TOP2A.UBE2C | BARD1 | 0.50 | FG.EC.GSTA2.AADAC | FERMT1 | 0.30 | FG.Glial.IER2.JUNB | TSPAN11 | 1.42 |
| FG.B/DZ.TOP2A.UBE2C | DBNL | 0.50 | FG.EC.GSTA2.AADAC | ATP2A2 | 0.30 | FG.Glial.IER2.JUNB | MAP1B | 1.37 |
| FG.B/DZ.TOP2A.UBE2C | PXMP2 | 0.50 | FG.EC.GSTA2.AADAC | ERRFI1 | 0.30 | FG.Glial.IER2.JUNB | CMTM5 | 1.30 |
| FG.B/DZ.TOP2A.UBE2C | POLR2G | 0.50 | FG.EC.GSTA2.AADAC | BCL2L14 | 0.30 | FG.Glial.IER2.JUNB | XKR4 | 1.28 |
| FG.B/DZ.TOP2A.UBE2C | TCEB1 | 0.50 | FG.EC.GSTA2.AADAC | GPAT3 | 0.30 | FG.Glial.IER2.JUNB | CADM2 | 1.27 |
| FG.B/DZ.TOP2A.UBE2C | RNASEH2C | 0.50 | FG.EC.GSTA2.AADAC | ABCA5 | 0.30 | FG.Glial.IER2.JUNB | SOX2 | 1.23 |
| FG.B/DZ.TOP2A.UBE2C | SUMO1 | 0.50 | FG.EC.GSTA2.AADAC | EIF4G1 | 0.30 | FG.Glial.IER2.JUNB | ST6GALNAC2 | 1.20 |
| FG.B/DZ.TOP2A.UBE2C | RAC2 | 0.50 | FG.EC.GSTA2.AADAC | SCTR | 0.30 | FG.Glial.IER2.JUNB | COL28A1 | 0.91 |
| FG.B/DZ.TOP2A.UBE2C | XRCC5 | 0.50 | FG.EC.GSTA2.AADAC | HSD11B2 | 0.29 | FG.Glial.IER2.JUNB | SNCA | 1.25 |
| FG.B/DZ.TOP2A.UBE2C | CHAF1A | 0.50 | FG.EC.GSTA2.AADAC | SOWAHA | 0.29 | FG.Glial.IER2.JUNB | NRXN3 | 0.87 |
| FG.B/DZ.TOP2A.UBE2C | ANAPC15 | 0.50 | FG.EC.GSTA2.AADAC | TNFRSF11A | 0.29 | FG.Glial.IER2.JUNB | LRRTM1 | 0.94 |
| FG.B/DZ.TOP2A.UBE2C | PSMA5 | 0.50 | FG.EC.GSTA2.AADAC | MPP5 | 0.29 | FG.Glial.IER2.JUNB | PCDH9 | 1.44 |
| FG.B/DZ.TOP2A.UBE2C | CEP57 | 0.50 | FG.EC.GSTA2.AADAC | FAM120A | 0.29 | FG.Glial.MS4A6A.MT-CO2 | CRYAB | 2.82 |
| FG.B/DZ.TOP2A.UBE2C | PAICS | 0.50 | FG.EC.GSTA2.AADAC | CEBPG | 0.29 | FG.Glial.MS4A6A.MT-CO2 | SPP1 | 2.55 |
| FG.B/DZ.TOP2A.UBE2C | SNRPA | 0.50 | FG.EC.GSTA2.AADAC | ANXA11 | 0.29 | FG.Glial.MS4A6A.MT-CO2 | PLP1 | 2.52 |
| FG.B/DZ.TOP2A.UBE2C | TROAP | 0.50 | FG.EC.GSTA2.AADAC | SLC44A1 | 0.29 | FG.Glial.MS4A6A.MT-CO2 | S100B | 2.35 |
| FG.B/DZ.TOP2A.UBE2C | CDKN2C | 0.49 | FG.EC.GSTA2.AADAC | HDGF | 0.29 | FG.Glial.MS4A6A.MT-CO2 | NRXN1 | 2.25 |
| FG.B/DZ.TOP2A.UBE2C | DNAJC8 | 0.49 | FG.EC.GSTA2.AADAC | CD151 | 0.29 | FG.Glial.MS4A6A.MT-CO2 | LGI4 | 2.22 |
| FG.B/DZ.TOP2A.UBE2C | NEK2 | 0.49 | FG.EC.GSTA2.AADAC | NR2F6 | 0.29 | FG.Glial.MS4A6A.MT-CO2 | SCN7A | 2.10 |
| FG.B/DZ.TOP2A.UBE2C | PSMC3 | 0.49 | FG.EC.GSTA2.AADAC | C6orf222 | 0.29 | FG.Glial.MS4A6A.MT-CO2 | CDH19 | 1.92 |
| FG.B/DZ.TOP2A.UBE2C | CSNK2B | 0.49 | FG.EC.GSTA2.AADAC | FAM84A | 0.29 | FG.Glial.S100A10.IGHA2 | CRYAB | 3.95 |
| FG.B/DZ.TOP2A.UBE2C | TCP1 | 0.49 | FG.EC.GSTA2.AADAC | F2RL1 | 0.29 | FG.Glial.S100A10.IGHA2 | SPARC | 3.29 |
| FG.B/DZ.TOP2A.UBE2C | NUP37 | 0.49 | FG.EC.GSTA2.AADAC | MOGAT3 | 0.29 | FG.Glial.S100A10.IGHA2 | FXYD1 | 3.27 |
| FG.B/DZ.TOP2A.UBE2C | RAD51C | 0.49 | FG.EC.GSTA2.AADAC | MYO5B | 0.29 | FG.Glial.S100A10.IGHA2 | S100B | 3.17 |
| FG.B/DZ.TOP2A.UBE2C | WDR1 | 0.49 | FG.EC.GSTA2.AADAC | TNFRSF1A | 0.29 | FG.Glial.S100A10.IGHA2 | PLP1 | 2.83 |
| FG.B/DZ.TOP2A.UBE2C | PMVK | 0.49 | FG.EC.GSTA2.AADAC | PNKD | 0.29 | FG.Glial.S100A10.IGHA2 | GPM6B | 2.77 |
| FG.B/DZ.TOP2A.UBE2C | CEP55 | 0.49 | FG.EC.GSTA2.AADAC | IDNK | 0.29 | FG.Glial.S100A10.IGHA2 | SPP1 | 2.76 |
| FG.B/DZ.TOP2A.UBE2C | GRB2 | 0.49 | FG.EC.GSTA2.AADAC | KLF4 | 0.29 | FG.Glial.S100A10.IGHA2 | PMP22 | 2.76 |
| FG.B/DZ.TOP2A.UBE2C | PNRC2 | 0.49 | FG.EC.GSTA2.AADAC | CPNE3 | 0.29 | FG.Glial.S100A10.IGHA2 | SEMA3B | 2.43 |
| FG.B/DZ.TOP2A.UBE2C | ELAVL1 | 0.48 | FG.EC.GSTA2.AADAC | FHL2 | 0.29 | FG.Glial.S100A10.IGHA2 | LGI4 | 2.43 |
| FG.B/DZ.TOP2A.UBE2C | SAC3D1 | 0.48 | FG.EC.GSTA2.AADAC | ARF5 | 0.29 | FG.Glial.S100A10.IGHA2 | GFRA3 | 2.30 |
| FG.B/DZ.TOP2A.UBE2C | MRPL13 | 0.48 | FG.EC.GSTA2.AADAC | SLC30A10 | 0.29 | FG.Glial.S100A10.IGHA2 | MPZ | 2.22 |
| FG.B/DZ.TOP2A.UBE2C | SMIM20 | 0.48 | FG.EC.GSTA2.AADAC | ANKS4B | 0.29 | FG.Glial.S100A10.IGHA2 | NRXN1 | 2.10 |
| FG.B/DZ.TOP2A.UBE2C | HAT1 | 0.48 | FG.EC.GSTA2.AADAC | BAG1 | 0.29 | FG.Glial.S100A10.IGHA2 | SCN7A | 2.09 |
| FG.B/DZ.TOP2A.UBE2C | MAD2L2 | 0.48 | FG.EC.GSTA2.AADAC | SLC25A20 | 0.29 | FG.Glial.S100A10.IGHA2 | MYOT | 2.01 |
| FG.B/DZ.TOP2A.UBE2C | UBE2D2 | 0.48 | FG.EC.GSTA2.AADAC | PSMA3 | 0.29 | FG.Glial.S100A10.IGHA2 | NRN1 | 1.94 |
| FG.B/DZ.TOP2A.UBE2C | CDKN2D | 0.48 | FG.EC.GSTA2.AADAC | ARCN1 | 0.28 | FG.Glial.S100A10.IGHA2 | TSPAN11 | 1.70 |
| FG.B/DZ.TOP2A.UBE2C | AHSA1 | 0.48 | FG.EC.GSTA2.AADAC | RGN | 0.28 | FG.Glial.S100A10.IGHA2 | PCSK2 | 1.54 |
| FG.B/DZ.TOP2A.UBE2C | PHF6 | 0.48 | FG.EC.GSTA2.AADAC | RMDN1 | 0.28 | FG.Glial.S100A10.IGHA2 | CDH19 | 1.47 |
| FG.B/DZ.TOP2A.UBE2C | CBX1 | 0.48 | FG.EC.GSTA2.AADAC | HIBADH | 0.28 | FG.Glial.S100A10.IGHA2 | ST6GALNAC2 | 1.45 |
| FG.B/DZ.TOP2A.UBE2C | NFYB | 0.48 | FG.EC.GSTA2.AADAC | SLC16A1 | 0.28 | FG.Glial.S100A10.IGHA2 | LRRTM1 | 1.45 |
| FG.B/DZ.TOP2A.UBE2C | CCT3 | 0.48 | FG.EC.GSTA2.AADAC | TTC38 | 0.28 | FG.Glial.S100A10.IGHA2 | FOXD3-AS1 | 1.31 |
| FG.B/DZ.TOP2A.UBE2C | MTCH2 | 0.48 | FG.EC.GSTA2.AADAC | MYO1D | 0.28 | FG.Glial.S100A10.IGHA2 | XKR4 | 1.27 |
| FG.B/DZ.TOP2A.UBE2C | CKAP2L | 0.48 | FG.EC.GSTA2.AADAC | DYNLT1 | 0.28 | FG.Glial.S100A10.IGHA2 | NTM | 1.27 |
| FG.B/DZ.TOP2A.UBE2C | COMMD4 | 0.48 | FG.EC.GSTA2.AADAC | PHYHIPL | 0.28 | FG.Glial.S100A10.IGHA2 | IGFBP7 | 2.57 |
| FG.B/DZ.TOP2A.UBE2C | TERF2 | 0.47 | FG.EC.GSTA2.AADAC | HRSP12 | 0.28 | FG.Glial.S100A10.IGHA2 | L1CAM | 1.20 |
| FG.B/DZ.TOP2A.UBE2C | ARL5A | 0.47 | FG.EC.GSTA2.AADAC | MTMR11 | 0.28 | FG.Glial.S100A10.IGHA2 | SLITRK6 | 1.35 |
| FG.B/DZ.TOP2A.UBE2C | TAF9 | 0.47 | FG.EC.GSTA2.AADAC | THAP4 | 0.28 | FG.Glial/fDC.FDCSP.CXCL13 | FDCSP | 5.79 |
| FG.B/DZ.TOP2A.UBE2C | COPS3 | 0.47 | FG.EC.GSTA2.AADAC | B3GALT5 | 0.28 | FG.Glial/fDC.FDCSP.CXCL13 | CXCL13 | 3.68 |
| FG.B/DZ.TOP2A.UBE2C | ZFAND6 | 0.47 | FG.EC.GSTA2.AADAC | EHF | 0.28 | FG.Glial/fDC.FDCSP.CXCL13 | CR2 | 3.07 |
| FG.B/DZ.TOP2A.UBE2C | UGP2 | 0.47 | FG.EC.GSTA2.AADAC | SH3BGRL2 | 0.28 | FG.Glial/fDC.FDCSP.CXCL13 | CSTA | 2.97 |
| FG.B/DZ.TOP2A.UBE2C | RAP1B | 0.47 | FG.EC.GSTA2.AADAC | EXT1 | 0.28 | FG.Glial/fDC.FDCSP.CXCL13 | SERPINE2 | 2.78 |
| FG.B/DZ.TOP2A.UBE2C | SUZ12 | 0.47 | FG.EC.GSTA2.AADAC | TXN2 | 0.28 | FG.Glial/fDC.FDCSP.CXCL13 | LTF | 2.63 |
| FG.B/DZ.TOP2A.UBE2C | EWSR1 | 0.47 | FG.EC.GSTA2.AADAC | DNAJC22 | 0.28 | FG.Glial/fDC.FDCSP.CXCL13 | FCAMR | 2.28 |
| FG.B/DZ.TOP2A.UBE2C | MAGOH | 0.46 | FG.EC.GSTA2.AADAC | KIAA1522 | 0.28 | FG.Glial/fDC.FDCSP.CXCL13 | S100A1 | 1.98 |
| FG.B/DZ.TOP2A.UBE2C | RPS7 | 0.46 | FG.EC.GSTA2.AADAC | DNPEP | 0.28 | FG.Glial/fDC.FDCSP.CXCL13 | NPPC | 1.98 |
| FG.B/DZ.TOP2A.UBE2C | PHF5A | 0.46 | FG.EC.GSTA2.AADAC | KIF5B | 0.28 | FG.Glial/fDC.FDCSP.CXCL13 | CHN1 | 1.80 |
| FG.B/DZ.TOP2A.UBE2C | CCDC88A | 0.46 | FG.EC.GSTA2.AADAC | PPP2CB | 0.28 | FG.Glial/fDC.FDCSP.CXCL13 | PDLIM4 | 1.77 |
| FG.B/DZ.TOP2A.UBE2C | LINC00877 | 0.46 | FG.EC.GSTA2.AADAC | DDRGK1 | 0.28 | FG.Glial/fDC.FDCSP.CXCL13 | SERPINA5 | 1.64 |
| FG.B/DZ.TOP2A.UBE2C | COMMD8 | 0.46 | FG.EC.GSTA2.AADAC | RP11-119D9.1 | 0.28 | FG.Glial/fDC.FDCSP.CXCL13 | TMEM163 | 1.40 |
| FG.B/DZ.TOP2A.UBE2C | RAB30 | 0.46 | FG.EC.GSTA2.AADAC | PFKFB2 | 0.28 | FG.Glial/fDC.FDCSP.CXCL13 | NPB | 1.40 |
| FG.B/DZ.TOP2A.UBE2C | LYPLAL1 | 0.46 | FG.EC.GSTA2.AADAC | ACBD5 | 0.28 | FG.Glial/fDC.FDCSP.CXCL13 | RGS9 | 1.27 |
| FG.B/DZ.TOP2A.UBE2C | FERMT3 | 0.46 | FG.EC.GSTA2.AADAC | NHSL1 | 0.28 | FG.Glial/fDC.FDCSP.CXCL13 | ANKRD29 | 1.24 |
| FG.B/DZ.TOP2A.UBE2C | CCDC34 | 0.46 | FG.EC.GSTA2.AADAC | RAVER2 | 0.28 | FG.Glial/fDC.FDCSP.CXCL13 | TF | 1.04 |
| FG.B/DZ.TOP2A.UBE2C | RHOH | 0.46 | FG.EC.GSTA2.AADAC | TMEM205 | 0.28 | FG.Glial/fDC.FDCSP.CXCL13 | S1PR3 | 1.00 |
| FG.B/DZ.TOP2A.UBE2C | SNX5 | 0.46 | FG.EC.GSTA2.AADAC | VAMP3 | 0.28 | FG.Glial/fDC.FDCSP.CXCL13 | PRUNE2 | 0.95 |
| FG.B/DZ.TOP2A.UBE2C | SYK | 0.46 | FG.EC.GSTA2.AADAC | SDF4 | 0.28 | FG.Glial/fDC.FDCSP.CXCL13 | PRELP | 0.85 |
| FG.B/DZ.TOP2A.UBE2C | MELK | 0.45 | FG.EC.GSTA2.AADAC | TMEM150B | 0.28 | FG.Glial/fDC.FDCSP.CXCL13 | ALPK2 | 0.84 |
| FG.B/DZ.TOP2A.UBE2C | TUBG1 | 0.45 | FG.EC.GSTA2.AADAC | PBDC1 | 0.28 | FG.Glial/fDC.FDCSP.CXCL13 | TNNT3 | 0.82 |
| FG.B/DZ.TOP2A.UBE2C | CDC6 | 0.45 | FG.EC.GSTA2.AADAC | ERBB2 | 0.28 | FG.Glial/fDC.FDCSP.CXCL13 | GNAL | 0.81 |
| FG.B/DZ.TOP2A.UBE2C | UCHL1 | 0.45 | FG.EC.GSTA2.AADAC | CDC42EP4 | 0.28 | FG.Glial/fDC.FDCSP.CXCL13 | RBPMS-AS1 | 0.77 |
| FG.B/DZ.TOP2A.UBE2C | SUPT16H | 0.45 | FG.EC.GSTA2.AADAC | NOSTRIN | 0.28 | FG.Glial/fDC.FDCSP.CXCL13 | PCOLCE2 | 0.73 |
| FG.B/DZ.TOP2A.UBE2C | SRSF1 | 0.45 | FG.EC.GSTA2.AADAC | MRPL27 | 0.27 | FG.Glial/fDC.FDCSP.CXCL13 | CELF4 | 0.69 |
| FG.B/DZ.TOP2A.UBE2C | CD79A | 0.45 | FG.EC.GSTA2.AADAC | THOC7 | 0.27 | FG.Glial/fDC.FDCSP.CXCL13 | STAR | 0.62 |
| FG.B/DZ.TOP2A.UBE2C | IFI16 | 0.45 | FG.EC.GSTA2.AADAC | LEAP2 | 0.27 | FG.Glial/fDC.FDCSP.CXCL13 | RP11-283G6.4 | 0.51 |
| FG.B/DZ.TOP2A.UBE2C | SUMO3 | 0.45 | FG.EC.GSTA2.AADAC | CTSZ | 0.27 | FG.Glial/fDC.FDCSP.CXCL13 | CAPN6 | 0.50 |
| FG.B/DZ.TOP2A.UBE2C | ILF3 | 0.45 | FG.EC.GSTA2.AADAC | CGN | 0.27 | FG.Glial/fDC.FDCSP.CXCL13 | VCAM1 | 2.36 |
| FG.B/DZ.TOP2A.UBE2C | CDK4 | 0.45 | FG.EC.GSTA2.AADAC | LCOR | 0.27 | FG.Goblet.FCGBP.SPINK4 | SPINK4 | 4.41 |
| FG.B/DZ.TOP2A.UBE2C | DCP2 | 0.45 | FG.EC.GSTA2.AADAC | DNAJA4 | 0.27 | FG.Goblet.FCGBP.SPINK4 | FCGBP | 3.88 |
| FG.B/DZ.TOP2A.UBE2C | SPDL1 | 0.45 | FG.EC.GSTA2.AADAC | TWF1 | 0.27 | FG.Goblet.FCGBP.SPINK4 | CLCA1 | 3.45 |
| FG.B/DZ.TOP2A.UBE2C | SIT1 | 0.44 | FG.EC.GSTA2.AADAC | B3GNT8 | 0.27 | FG.Goblet.FCGBP.SPINK4 | ITLN1 | 2.75 |
| FG.B/DZ.TOP2A.UBE2C | CCDC18 | 0.44 | FG.EC.GSTA2.AADAC | NCOA1 | 0.27 | FG.Goblet.FCGBP.SPINK4 | REG4 | 2.58 |
| FG.B/DZ.TOP2A.UBE2C | BAZ1B | 0.44 | FG.EC.GSTA2.AADAC | CASP1 | 0.27 | FG.Goblet.FCGBP.SPINK4 | REP15 | 1.18 |
| FG.B/DZ.TOP2A.UBE2C | CHEK1 | 0.44 | FG.EC.GSTA2.AADAC | LRPPRC | 0.27 | FG.Goblet.ITLN1.SPINK4 | TFF3 | 5.20 |
| FG.B/DZ.TOP2A.UBE2C | PRKRA | 0.44 | FG.EC.GSTA2.AADAC | LTBR | 0.27 | FG.Goblet.ITLN1.SPINK4 | SPINK4 | 4.66 |
| FG.B/DZ.TOP2A.UBE2C | RAD23B | 0.44 | FG.EC.GSTA2.AADAC | PDLIM1 | 0.27 | FG.Goblet.ITLN1.SPINK4 | CLCA1 | 3.82 |
| FG.B/DZ.TOP2A.UBE2C | MRPS16 | 0.44 | FG.EC.GSTA2.AADAC | HSD17B12 | 0.27 | FG.Goblet.ITLN1.SPINK4 | REG4 | 3.31 |
| FG.B/DZ.TOP2A.UBE2C | ZNF106 | 0.44 | FG.EC.GSTA2.AADAC | C6orf136 | 0.27 | FG.Goblet.ITLN1.SPINK4 | ITLN1 | 3.31 |
| FG.B/DZ.TOP2A.UBE2C | PSMG2 | 0.44 | FG.EC.GSTA2.AADAC | MATN2 | 0.27 | FG.Goblet.ITLN1.SPINK4 | FCGBP | 3.24 |
| FG.B/DZ.TOP2A.UBE2C | CPSF6 | 0.44 | FG.EC.GSTA2.AADAC | MCUR1 | 0.27 | FG.Goblet.ITLN1.SPINK4 | RETNLB | 2.01 |
| FG.B/DZ.TOP2A.UBE2C | SNRNP70 | 0.44 | FG.EC.GSTA2.AADAC | PKP2 | 0.27 | FG.Goblet.ITLN1.SPINK4 | HEPACAM2 | 1.30 |
| FG.B/DZ.TOP2A.UBE2C | MRPL16 | 0.44 | FG.EC.GSTA2.AADAC | PSMB5 | 0.27 | FG.Goblet.ITLN1.SPINK4 | REP15 | 1.25 |
| FG.B/DZ.TOP2A.UBE2C | SSNA1 | 0.44 | FG.EC.GSTA2.AADAC | HMGCS1 | 0.27 | FG.Goblet.ITLN1.SPINK4 | LRRC26 | 1.02 |
| FG.B/DZ.TOP2A.UBE2C | MYL6B | 0.44 | FG.EC.GSTA2.AADAC | SHMT1 | 0.27 | FG.Goblet.ITLN1.SPINK4 | RAP1GAP | 0.90 |
| FG.B/DZ.TOP2A.UBE2C | RRP7A | 0.44 | FG.EC.GSTA2.AADAC | SDSL | 0.27 | FG.Goblet.ITLN1.SPINK4 | MB | 0.88 |
| FG.B/DZ.TOP2A.UBE2C | TXNL4A | 0.44 | FG.EC.GSTA2.AADAC | TALDO1 | 0.27 | FG.Goblet.ITLN1.SPINK4 | RP11-234B24.2 | 0.82 |
| FG.B/DZ.TOP2A.UBE2C | EXOSC3 | 0.44 | FG.EC.GSTA2.AADAC | GSTM3 | 0.27 | FG.Goblet.ITLN1.SPINK4 | ATOH1 | 0.68 |
| FG.B/DZ.TOP2A.UBE2C | ACTL6A | 0.44 | FG.EC.GSTA2.AADAC | MDK | 0.27 | FG.Goblet.ITLN1.SPINK4 | ABCA4 | 0.57 |
| FG.B/DZ.TOP2A.UBE2C | HCLS1 | 0.44 | FG.EC.GSTA2.AADAC | SMCO4 | 0.27 | FG.Goblet.ITLN1.SPINK4 | FOXA3 | 1.02 |
| FG.B/DZ.TOP2A.UBE2C | DEPDC1B | 0.44 | FG.EC.GSTA2.AADAC | PCYT2 | 0.27 | FG.Goblet.ITLN1.ZG16 | CLCA1 | 4.67 |
| FG.B/DZ.TOP2A.UBE2C | BLK | 0.44 | FG.EC.GSTA2.AADAC | MAPKAPK5 | 0.27 | FG.Goblet.ITLN1.ZG16 | FCGBP | 3.84 |
| FG.B/DZ.TOP2A.UBE2C | GOT2 | 0.43 | FG.EC.GSTA2.AADAC | NT5C | 0.26 | FG.Goblet.ITLN1.ZG16 | SPINK4 | 3.61 |
| FG.B/DZ.TOP2A.UBE2C | MED30 | 0.43 | FG.EC.GSTA2.AADAC | MT-ND6 | 0.26 | FG.Goblet.ITLN1.ZG16 | ITLN1 | 3.39 |
| FG.B/DZ.TOP2A.UBE2C | ATP6V1E1 | 0.43 | FG.EC.GSTA2.AADAC | RTKN | 0.26 | FG.Goblet.ITLN1.ZG16 | RETNLB | 2.43 |
| FG.B/DZ.TOP2A.UBE2C | UBA2 | 0.43 | FG.EC.GSTA2.AADAC | PDK2 | 0.26 | FG.Goblet.ITLN1.ZG16 | REP15 | 1.32 |
| FG.B/DZ.TOP2A.UBE2C | FIBP | 0.43 | FG.EC.GSTA2.AADAC | ST6GALNAC1 | 0.26 | FG.Goblet.ITLN1.ZG16 | HEPACAM2 | 1.24 |
| FG.B/DZ.TOP2A.UBE2C | PIF1 | 0.43 | FG.EC.GSTA2.AADAC | PTTG1IP | 0.26 | FG.Goblet.ITLN1.ZG16 | BCAS1 | 1.23 |
| FG.B/DZ.TOP2A.UBE2C | BACH2 | 0.43 | FG.EC.GSTA2.AADAC | LRRFIP2 | 0.26 | FG.Goblet.ITLN1.ZG16 | RP11-234B24.2 | 0.83 |
| FG.B/DZ.TOP2A.UBE2C | ARHGAP11A | 0.43 | FG.EC.GSTA2.AADAC | PDXK | 0.26 | FG.Goblet.ITLN1.ZG16 | ABCA4 | 0.83 |
| FG.B/DZ.TOP2A.UBE2C | HSBP1 | 0.42 | FG.EC.GSTA2.AADAC | SSFA2 | 0.26 | FG.Goblet.ITLN1.ZG16 | ATOH1 | 0.79 |
| FG.B/DZ.TOP2A.UBE2C | KIAA1524 | 0.42 | FG.EC.GSTA2.AADAC | MTRNR2L12 | 0.26 | FG.Goblet.SPINK4.ITLN1 | TFF3 | 3.94 |
| FG.B/DZ.TOP2A.UBE2C | MEF2C | 0.42 | FG.EC.GSTA2.AADAC | OCLN | 0.26 | FG.Goblet.SPINK4.ITLN1 | SPINK4 | 3.42 |
| FG.B/DZ.TOP2A.UBE2C | RUVBL2 | 0.42 | FG.EC.GSTA2.AADAC | SLC35D2 | 0.26 | FG.Goblet.SPINK4.ITLN1 | CLCA1 | 2.69 |
| FG.B/DZ.TOP2A.UBE2C | C4orf32 | 0.42 | FG.EC.GSTA2.AADAC | FAM177A1 | 0.26 | FG.Goblet.SPINK4.ITLN1 | FCGBP | 2.27 |
| FG.B/DZ.TOP2A.UBE2C | SPC24 | 0.42 | FG.EC.GSTA2.AADAC | DHRS4 | 0.26 | FG.Goblet.SPINK4.ITLN1 | REG4 | 2.20 |
| FG.B/DZ.TOP2A.UBE2C | PCNP | 0.42 | FG.EC.GSTA2.AADAC | OGDH | 0.26 | FG.Goblet.SPINK4.ITLN1 | ITLN1 | 2.20 |
| FG.B/DZ.TOP2A.UBE2C | PRKCD | 0.42 | FG.EC.GSTA2.AADAC | CEACAM1 | 0.26 | FG.Goblet.SPINK4.ITLN1 | GPX2 | 1.49 |
| FG.B/DZ.TOP2A.UBE2C | FAM49B | 0.42 | FG.EC.GSTA2.AADAC | CYP2J2 | 0.26 | FG.Goblet.SPINK4.ITLN1 | RNASE1 | 1.40 |
| FG.B/DZ.TOP2A.UBE2C | GLRX5 | 0.42 | FG.EC.GSTA2.AADAC | HGD | 0.26 | FG.Goblet.SPINK4.ITLN1 | PPP1R1B | 1.15 |
| FG.B/DZ.TOP2A.UBE2C | GGNBP2 | 0.42 | FG.EC.GSTA2.AADAC | COQ9 | 0.26 | FG.Goblet.SPINK4.ITLN1 | RETNLB | 1.12 |
| FG.B/DZ.TOP2A.UBE2C | ATAD5 | 0.41 | FG.EC.GSTA2.AADAC | KIF1C | 0.26 | FG.Goblet.SPINK4.ITLN1 | ST6GALNAC1 | 1.11 |
| FG.B/DZ.TOP2A.UBE2C | HIST2H2AC | 0.41 | FG.EC.GSTA2.AADAC | GLOD5 | 0.26 | FG.Goblet.SPINK4.ITLN1 | PLA2G2A | 1.11 |
| FG.B/DZ.TOP2A.UBE2C | COPS8 | 0.41 | FG.EC.GSTA2.AADAC | BCL10 | 0.26 | FG.Goblet.SPINK4.ITLN1 | KLK1 | 0.93 |
| FG.B/DZ.TOP2A.UBE2C | SHMT2 | 0.41 | FG.EC.GSTA2.AADAC | KCNE3 | 0.26 | FG.Goblet.SPINK4.ITLN1 | KCNJ13 | 0.74 |
| FG.B/DZ.TOP2A.UBE2C | PHAX | 0.41 | FG.EC.GSTA2.AADAC | CASP6 | 0.26 | FG.Goblet.SPINK4.ITLN1 | HEPACAM2 | 0.72 |
| FG.B/DZ.TOP2A.UBE2C | RPS2 | 0.41 | FG.EC.GSTA2.AADAC | SLC46A3 | 0.26 | FG.Goblet.SPINK4.ITLN1 | ATOH1 | 0.68 |
| FG.B/DZ.TOP2A.UBE2C | PAX5 | 0.41 | FG.EC.GSTA2.AADAC | MAP3K13 | 0.26 | FG.Goblet.SPINK4.ITLN1 | EPHB2 | 0.64 |
| FG.B/DZ.TOP2A.UBE2C | NUDCD2 | 0.41 | FG.EC.GSTA2.AADAC | NAGS | 0.26 | FG.Goblet.SPINK4.ITLN1 | FOXA3 | 0.63 |
| FG.B/DZ.TOP2A.UBE2C | CD180 | 0.41 | FG.EC.GSTA2.AADAC | CLTC | 0.26 | FG.Goblet.SPINK4.ITLN1 | REP15 | 0.56 |
| FG.B/DZ.TOP2A.UBE2C | BRWD1 | 0.41 | FG.EC.GSTA2.AADAC | HACD3 | 0.26 | FG.Goblet.SPINK4.ITLN1 | CREB3L1 | 0.55 |
| FG.B/DZ.TOP2A.UBE2C | PRIM1 | 0.41 | FG.EC.GSTA2.AADAC | MICALL2 | 0.26 | FG.Goblet.SPINK4.ITLN1 | TNNC2 | 0.55 |
| FG.B/DZ.TOP2A.UBE2C | SYVN1 | 0.41 | FG.EC.GSTA2.AADAC | ME2 | 0.26 | FG.Goblet.SPINK4.ITLN1 | URAD | 0.54 |
| FG.B/DZ.TOP2A.UBE2C | WHSC1 | 0.41 | FG.EC.GSTA2.AADAC | ANK3 | 0.26 | FG.Goblet.SPINK4.ITLN1 | RP11-357H14.17 | 0.53 |
| FG.B/DZ.TOP2A.UBE2C | RMDN1 | 0.41 | FG.EC.GSTA2.AADAC | ABCD3 | 0.26 | FG.Goblet.SPINK4.ITLN1 | LRRC26 | 0.52 |
| FG.B/DZ.TOP2A.UBE2C | NDC1 | 0.41 | FG.EC.GSTA2.AADAC | BAIAP2L1 | 0.26 | FG.Goblet.SPINK4.ITLN1 | HOXB7 | 0.50 |
| FG.B/DZ.TOP2A.UBE2C | PTBP1 | 0.41 | FG.EC.GSTA2.AADAC | TNIK | 0.25 | FG.Goblet.SPINK4.ITLN1 | RP11-234B24.2 | 0.49 |
| FG.B/DZ.TOP2A.UBE2C | SNTA1 | 0.41 | FG.EC.GSTA2.AADAC | CEBPA | 0.25 | FG.Goblet.SPINK4.ITLN1 | RAP1GAP | 0.42 |
| FG.B/DZ.TOP2A.UBE2C | GTF2E2 | 0.41 | FG.EC.GSTA2.AADAC | REEP3 | 0.25 | FG.Goblet.SPINK4.ITLN1 | EPHB3 | 0.38 |
| FG.B/DZ.TOP2A.UBE2C | CKAP5 | 0.41 | FG.EC.GSTA2.AADAC | CASP3 | 0.25 | FG.Goblet.SPINK4.ITLN1 | C10orf99 | 0.38 |
| FG.B/DZ.TOP2A.UBE2C | RAD17 | 0.41 | FG.EC.GSTA2.AADAC | SCO2 | 0.25 | FG.Goblet.SPINK4.ITLN1 | ABCA4 | 0.35 |
| FG.B/DZ.TOP2A.UBE2C | KPNB1 | 0.40 | FG.EC.GSTA2.AADAC | SERTAD3 | 0.25 | FG.Goblet.SPINK4.ITLN1 | WFDC2 | 0.35 |
| FG.B/DZ.TOP2A.UBE2C | WEE1 | 0.40 | FG.EC.GSTA2.AADAC | GUCD1 | 0.25 | FG.Goblet.SPINK4.ITLN1 | ERN2 | 0.34 |
| FG.B/DZ.TOP2A.UBE2C | ITSN2 | 0.40 | FG.EC.GSTA2.AADAC | EPS8L2 | 0.25 | FG.Goblet.SPINK4.ITLN1 | CA12 | 0.34 |
| FG.B/DZ.TOP2A.UBE2C | FAM111A | 0.40 | FG.EC.GSTA2.AADAC | GSTO2 | 0.25 | FG.Goblet.SPINK4.ITLN1 | HOXB9 | 0.33 |
| FG.B/DZ.TOP2A.UBE2C | MCM2 | 0.40 | FG.EC.GSTA2.AADAC | PDGFA | 0.25 | FG.Goblet.SPINK4.ITLN1 | FOXA2 | 0.31 |
| FG.B/DZ.TOP2A.UBE2C | AIM2 | 0.40 | FG.EC.GSTA2.AADAC | FBXL15 | 0.25 | FG.Goblet.SPINK4.ITLN1 | BCAS1 | 0.30 |
| FG.B/DZ.TOP2A.UBE2C | STAP1 | 0.40 | FG.EC.GSTA2.AADAC | KCTD3 | 0.25 | FG.Goblet.SPINK4.ITLN1 | RP11-519G16.5 | 0.28 |
| FG.B/DZ.TOP2A.UBE2C | CCM2 | 0.40 | FG.EC.GSTA2.AADAC | FGD4 | 0.25 | FG.Goblet.SPINK4.ITLN1 | ZG16B | 0.28 |
| FG.B/DZ.TOP2A.UBE2C | FANCI | 0.40 | FG.EC.GSTA2.AADAC | MALRD1 | 0.25 | FG.Goblet.SPINK4.ITLN1 | LINC00261 | 0.28 |
| FG.B/DZ.TOP2A.UBE2C | C20orf24 | 0.39 | FG.EC.GSTA2.AADAC | DAB1 | 0.25 | FG.Goblet.SPINK4.ITLN1 | SCNN1A | 0.27 |
| FG.B/DZ.TOP2A.UBE2C | C16orf87 | 0.39 | FG.EC.GSTA2.AADAC | PIP5K1B | 0.25 | FG.Goblet.SPINK4.ITLN1 | MB | 0.26 |
| FG.B/DZ.TOP2A.UBE2C | EED | 0.39 | FG.EC.GSTA2.AADAC | ELMO3 | 0.25 | FG.Goblet.SPINK4.ITLN1 | C2orf82 | 0.58 |
| FG.B/DZ.TOP2A.UBE2C | LUC7L2 | 0.39 | FG.EC.GSTA2.AADAC | TFRC | 0.25 | FG.Goblet.SPINK4.ITLN1 | SLC12A2 | 1.76 |
| FG.B/DZ.TOP2A.UBE2C | CBFB | 0.39 | FG.EC.GSTA2.AADAC | NEAT1 | 0.69 | FG.Goblet.TFF2.MUC6 | TFF2 | 6.75 |
| FG.B/DZ.TOP2A.UBE2C | TADA3 | 0.39 | FG.EC.GSTA2.AADAC | TKT | 0.31 | FG.Goblet.TFF2.MUC6 | PGC | 5.21 |
| FG.B/DZ.TOP2A.UBE2C | GMPS | 0.39 | FG.EC.GSTA2.AADAC | PTRHD1 | 0.26 | FG.Goblet.TFF2.MUC6 | MUC6 | 4.94 |
| FG.B/DZ.TOP2A.UBE2C | ATG3 | 0.39 | FG.EC.GSTA2.AADAC | ROMO1 | 0.42 | FG.Goblet.TFF2.MUC6 | C6orf58 | 4.60 |
| FG.B/DZ.TOP2A.UBE2C | PIK3AP1 | 0.39 | FG.EC.GSTA2.AADAC | EIF3K | 0.67 | FG.Goblet.TFF2.MUC6 | RP11-1143G9.4 | 3.62 |
| FG.B/DZ.TOP2A.UBE2C | EXOSC8 | 0.39 | FG.EC.GSTA2.AADAC | NDUFB4 | 0.38 | FG.Goblet.TFF2.MUC6 | ZG16B | 2.11 |
| FG.B/DZ.TOP2A.UBE2C | EMC9 | 0.39 | FG.EC.KIAA0101.FAM111B | DMBT1 | 2.20 | FG.Goblet.TFF2.MUC6 | GP2 | 2.03 |
| FG.B/DZ.TOP2A.UBE2C | XPO1 | 0.39 | FG.EC.KIAA0101.FAM111B | MT1H | 1.86 | FG.Goblet.TFF2.MUC6 | MUC1 | 1.90 |
| FG.B/DZ.TOP2A.UBE2C | OIP5 | 0.39 | FG.EC.KIAA0101.FAM111B | MGST1 | 1.56 | FG.Goblet.TFF2.MUC6 | BPIFB1 | 1.73 |
| FG.B/DZ.TOP2A.UBE2C | BLNK | 0.38 | FG.EC.KIAA0101.FAM111B | RAMP1 | 1.37 | FG.Goblet.TFF2.MUC6 | LIPF | 1.64 |
| FG.B/DZ.TOP2A.UBE2C | BPTF | 0.38 | FG.EC.KIAA0101.FAM111B | SFN | 1.34 | FG.Goblet.TFF2.MUC6 | PDIA2 | 1.62 |
| FG.B/DZ.TOP2A.UBE2C | MTA3 | 0.38 | FG.EC.KIAA0101.FAM111B | MT1M | 1.23 | FG.Goblet.TFF2.MUC6 | AQP5 | 1.61 |
| FG.B/DZ.TOP2A.UBE2C | NCAPD2 | 0.38 | FG.EC.KIAA0101.FAM111B | CPS1 | 1.21 | FG.Goblet.TFF2.MUC6 | TCN1 | 1.60 |
| FG.B/DZ.TOP2A.UBE2C | MAPRE1 | 0.38 | FG.EC.KIAA0101.FAM111B | PPP1R1B | 1.02 | FG.Goblet.TFF2.MUC6 | CDR1-AS | 1.53 |
| FG.B/DZ.TOP2A.UBE2C | RGS19 | 0.38 | FG.EC.KIAA0101.FAM111B | GOLM1 | 0.82 | FG.Goblet.TFF2.MUC6 | FMOD | 1.52 |
| FG.B/DZ.TOP2A.UBE2C | MSH2 | 0.38 | FG.EC.KIAA0101.FAM111B | EMP2 | 0.79 | FG.Goblet.TFF2.MUC6 | KCNE2 | 1.46 |
| FG.B/DZ.TOP2A.UBE2C | SEMA4A | 0.38 | FG.EC.KIAA0101.FAM111B | TCEA3 | 0.77 | FG.Goblet.TFF2.MUC6 | C16orf89 | 1.42 |
| FG.B/DZ.TOP2A.UBE2C | TBC1D1 | 0.38 | FG.EC.KIAA0101.FAM111B | TYMS | 0.73 | FG.Goblet.TFF2.MUC6 | RP13-870H17.3 | 1.31 |
| FG.B/DZ.TOP2A.UBE2C | CSK | 0.38 | FG.EC.KIAA0101.FAM111B | GINS2 | 0.71 | FG.Goblet.TFF2.MUC6 | S100A1 | 1.27 |
| FG.B/DZ.TOP2A.UBE2C | ASRGL1 | 0.38 | FG.EC.KIAA0101.FAM111B | FAM111B | 0.62 | FG.Goblet.TFF2.MUC6 | LINC00261 | 1.22 |
| FG.B/DZ.TOP2A.UBE2C | MIS18A | 0.37 | FG.EC.KIAA0101.FAM111B | PLEK2 | 0.62 | FG.Goblet.TFF2.MUC6 | TFF1 | 1.04 |
| FG.B/DZ.TOP2A.UBE2C | TEX30 | 0.37 | FG.EC.KIAA0101.FAM111B | SCTR | 0.59 | FG.Goblet.TFF2.MUC6 | FUT9 | 1.00 |
| FG.B/DZ.TOP2A.UBE2C | INO80C | 0.37 | FG.EC.KIAA0101.FAM111B | AQP1 | 0.58 | FG.Goblet.TFF2.MUC6 | VSIG2 | 0.93 |
| FG.B/DZ.TOP2A.UBE2C | ARL6IP6 | 0.37 | FG.EC.KIAA0101.FAM111B | MT1A | 0.56 | FG.Goblet.TFF2.MUC6 | A4GNT | 0.91 |
| FG.B/DZ.TOP2A.UBE2C | E2F1 | 0.37 | FG.EC.KIAA0101.FAM111B | LAD1 | 0.54 | FG.Goblet.TFF2.MUC6 | SLPI | 0.89 |
| FG.B/DZ.TOP2A.UBE2C | BUB1B | 0.37 | FG.EC.KIAA0101.FAM111B | CDC6 | 0.50 | FG.Goblet.TFF2.MUC6 | SCG5 | 0.88 |
| FG.B/DZ.TOP2A.UBE2C | PKMYT1 | 0.37 | FG.EC.KIAA0101.FAM111B | EPHB2 | 0.45 | FG.Goblet.TFF2.MUC6 | SERPINA4 | 0.75 |
| FG.B/DZ.TOP2A.UBE2C | ARHGAP11B | 0.37 | FG.EC.KIAA0101.FAM111B | TRIM54 | 0.43 | FG.Goblet.TFF2.MUC6 | CLDN10 | 0.67 |
| FG.B/DZ.TOP2A.UBE2C | CNP | 0.37 | FG.EC.KIAA0101.FAM111B | TMEM171 | 0.41 | FG.Goblet.TFF2.MUC6 | CXCL17 | 0.47 |
| FG.B/DZ.TOP2A.UBE2C | RCCD1 | 0.37 | FG.EC.KIAA0101.FAM111B | CA9 | 0.37 | FG.Goblet.TFF2.MUC6 | SCGB2A1 | 0.35 |
| FG.B/DZ.TOP2A.UBE2C | LRRK1 | 0.37 | FG.EC.KIAA0101.FAM111B | C2orf82 | 0.54 | FG.Mac.APOE.PTGDS | LYZ | 3.29 |
| FG.B/DZ.TOP2A.UBE2C | NCAPH | 0.36 | FG.EC.KIAA0101.FAM111B | ADH1C | 1.84 | FG.Mac.APOE.PTGDS | PTGDS | 3.04 |
| FG.B/DZ.TOP2A.UBE2C | MGME1 | 0.36 | FG.EC.KIAA0101.FAM111B | OTC | 0.80 | FG.Mac.APOE.PTGDS | APOE | 3.03 |
| FG.B/DZ.TOP2A.UBE2C | PRR11 | 0.36 | FG.EC.KIAA0101.FAM111B | GPX2 | 0.78 | FG.Mac.APOE.PTGDS | CCL18 | 2.94 |
| FG.B/DZ.TOP2A.UBE2C | ASB13 | 0.36 | FG.EC.KIAA0101.FAM111B | CDX1 | 0.85 | FG.Mac.APOE.PTGDS | MMP9 | 2.91 |
| FG.B/DZ.TOP2A.UBE2C | CTCF | 0.36 | FG.EC.KIAA0101.FAM111B | TK1 | 0.78 | FG.Mac.APOE.PTGDS | APOC1 | 2.91 |
| FG.B/DZ.TOP2A.UBE2C | CD19 | 0.36 | FG.EC.KIAA0101.FAM111B | AGR2 | 1.76 | FG.Mac.APOE.PTGDS | MMP12 | 2.77 |
| FG.B/DZ.TOP2A.UBE2C | PAG1 | 0.36 | FG.EC.KIAA0101.FAM111B | TMEM97 | 0.44 | FG.Mac.APOE.PTGDS | C1QA | 2.66 |
| FG.B/DZ.TOP2A.UBE2C | ACSF3 | 0.36 | FG.EC.MT-CO2.MT-CO3 | MT-ND4L | 2.47 | FG.Mac.APOE.PTGDS | DNASE1L3 | 2.56 |
| FG.B/DZ.TOP2A.UBE2C | TCF3 | 0.36 | FG.EC.MT-CO2.MT-CO3 | DMBT1 | 1.94 | FG.Mac.APOE.PTGDS | C1QC | 2.54 |
| FG.B/DZ.TOP2A.UBE2C | CHTOP | 0.36 | FG.EC.MT-CO2.MT-CO3 | MT-ND6 | 1.51 | FG.Mac.APOE.PTGDS | C1QB | 2.46 |
| FG.B/DZ.TOP2A.UBE2C | FABP5 | 0.36 | FG.EC.MT-CO2.MT-CO3 | PIGR | 1.51 | FG.Mac.APOE.PTGDS | FUCA1 | 2.45 |
| FG.B/DZ.TOP2A.UBE2C | DAPP1 | 0.36 | FG.EC.MT-CO2.MT-CO3 | MTRNR2L12 | 1.50 | FG.Mac.APOE.PTGDS | SEPP1 | 2.45 |
| FG.B/DZ.TOP2A.UBE2C | FUBP1 | 0.35 | FG.EC.MT-CO2.MT-CO3 | ELF3 | 1.47 | FG.Mac.APOE.PTGDS | FTL | 2.44 |
| FG.B/DZ.TOP2A.UBE2C | ATXN10 | 0.35 | FG.EC.MT-CO2.MT-CO3 | MT1G | 1.25 | FG.Mac.APOE.PTGDS | AIF1 | 2.30 |
| FG.B/DZ.TOP2A.UBE2C | ITGB1BP1 | 0.35 | FG.EC.MT-CO2.MT-CO3 | REG1A | 1.20 | FG.Mac.APOE.PTGDS | NPC2 | 2.28 |
| FG.B/DZ.TOP2A.UBE2C | MAP4K1 | 0.35 | FG.EC.MT-CO2.MT-CO3 | MT-CO2 | 2.31 | FG.Mac.APOE.PTGDS | CD68 | 2.22 |
| FG.B/DZ.TOP2A.UBE2C | RPA1 | 0.35 | FG.EC.MT-CO2.MT-CO3 | MT-CO3 | 2.33 | FG.Mac.APOE.PTGDS | GPNMB | 2.20 |
| FG.B/DZ.TOP2A.UBE2C | PAQR4 | 0.35 | FG.EC.MT-CO2.MT-CO3 | MT-ND4 | 2.17 | FG.Mac.APOE.PTGDS | CSTA | 2.10 |
| FG.B/DZ.TOP2A.UBE2C | SPAG5 | 0.35 | FG.EC.MT-CO2.MT-CO3 | MT-CYB | 2.21 | FG.Mac.APOE.PTGDS | TYROBP | 2.07 |
| FG.B/DZ.TOP2A.UBE2C | ACAT2 | 0.35 | FG.EC.NPY.ADGRF1 | CPA2 | 2.46 | FG.Mac.APOE.PTGDS | CST3 | 2.04 |
| FG.B/DZ.TOP2A.UBE2C | DONSON | 0.35 | FG.EC.NPY.ADGRF1 | BEST4 | 2.40 | FG.Mac.APOE.PTGDS | SERPINF1 | 2.03 |
| FG.B/DZ.TOP2A.UBE2C | RCC1 | 0.34 | FG.EC.NPY.ADGRF1 | CA7 | 2.29 | FG.Mac.APOE.PTGDS | RP11-1143G9.4 | 2.01 |
| FG.B/DZ.TOP2A.UBE2C | MILR1 | 0.34 | FG.EC.NPY.ADGRF1 | HEPACAM2 | 1.88 | FG.Mac.APOE.PTGDS | FCER1G | 1.96 |
| FG.B/DZ.TOP2A.UBE2C | POLD4 | 0.34 | FG.EC.NPY.ADGRF1 | CTSE | 1.73 | FG.Mac.APOE.PTGDS | PLA2G2D | 1.95 |
| FG.B/DZ.TOP2A.UBE2C | POC1A | 0.34 | FG.EC.NPY.ADGRF1 | ADGRG4 | 1.72 | FG.Mac.APOE.PTGDS | IL18 | 1.91 |
| FG.B/DZ.TOP2A.UBE2C | PANK2 | 0.34 | FG.EC.NPY.ADGRF1 | CACNA2D1 | 1.08 | FG.Mac.APOE.PTGDS | TMEM176B | 1.82 |
| FG.B/DZ.TOP2A.UBE2C | FANCA | 0.34 | FG.EC.NPY.ADGRF1 | RHOV | 1.02 | FG.Mac.APOE.PTGDS | MS4A6A | 1.78 |
| FG.B/DZ.TOP2A.UBE2C | BFSP2 | 0.34 | FG.EC.NPY.ADGRF1 | S100P | 0.98 | FG.Mac.APOE.PTGDS | IGSF6 | 1.60 |
| FG.B/DZ.TOP2A.UBE2C | EHD1 | 0.34 | FG.EC.NPY.ADGRF1 | PROX1 | 0.96 | FG.Mac.APOE.PTGDS | LST1 | 1.57 |
| FG.B/DZ.TOP2A.UBE2C | BCL11A | 0.33 | FG.EC.NPY.ADGRF1 | NEURL1 | 0.91 | FG.Mac.APOE.PTGDS | LILRB4 | 1.55 |
| FG.B/DZ.TOP2A.UBE2C | EML6 | 0.33 | FG.EC.NPY.ADGRF1 | ANKRD22 | 0.88 | FG.Mac.APOE.PTGDS | PLA2G7 | 1.52 |
| FG.B/DZ.TOP2A.UBE2C | AC079767.4 | 0.33 | FG.EC.NPY.ADGRF1 | ADGRF1 | 0.71 | FG.Mac.APOE.PTGDS | ENPP2 | 1.50 |
| FG.B/DZ.TOP2A.UBE2C | DLEU2 | 0.33 | FG.EC.NPY.ADGRF1 | CITED1 | 0.54 | FG.Mac.APOE.PTGDS | CD14 | 1.45 |
| FG.B/DZ.TOP2A.UBE2C | DSCC1 | 0.33 | FG.EC.NPY.ADGRF1 | KRT72 | 0.51 | FG.Mac.APOE.PTGDS | NPL | 0.94 |
| FG.B/DZ.TOP2A.UBE2C | KIF4A | 0.33 | FG.EC.NPY.ADGRF1 | RP11-771K4.1 | 0.49 | FG.Mac.APOE.PTGDS | MMP1 | 0.81 |
| FG.B/DZ.TOP2A.UBE2C | PARPBP | 0.33 | FG.EC.NPY.ADGRF1 | C1orf168 | 0.49 | FG.Mac.APOE.PTGDS | C1orf54 | 1.15 |
| FG.B/DZ.TOP2A.UBE2C | TTK | 0.32 | FG.EC.NPY.ADGRF1 | RP11-404P21.3 | 0.35 | FG.Mac.APOE.PTGDS | CSF1R | 0.84 |
| FG.B/DZ.TOP2A.UBE2C | DSN1 | 0.32 | FG.EC.NPY.ADGRF1 | RP11-411K7.1 | 0.28 | FG.Mac.APOE.PTGDS | CTSD | 2.48 |
| FG.B/DZ.TOP2A.UBE2C | TIMELESS | 0.32 | FG.EC.OLFM4.LDHB | PPP1R1B | 1.15 | FG.Mac.APOE.PTGDS | PSAP | 2.37 |
| FG.B/DZ.TOP2A.UBE2C | SKA3 | 0.32 | FG.EC.OLFM4.LDHB | GPX2 | 1.07 | FG.Mac.APOE.PTGDS | IL1B | 1.36 |
| FG.B/DZ.TOP2A.UBE2C | USP34 | 0.32 | FG.EC.OLFM4.LDHB | GOLM1 | 1.00 | FG.Mac.APOE.PTGDS | ATOX1 | 2.51 |
| FG.B/DZ.TOP2A.UBE2C | CDC45 | 0.32 | FG.EC.OLFM4.LDHB | AQP1 | 0.59 | FG.Mac.APOE.PTGDS | MS4A4A | 1.15 |
| FG.B/DZ.TOP2A.UBE2C | RFC5 | 0.32 | FG.EC.OLFM4.LDHB | CA9 | 0.46 | FG.Mac.C1QB.SEPP1 | C1QA | 3.64 |
| FG.B/DZ.TOP2A.UBE2C | SOCS1 | 0.31 | FG.EC.OLFM4.LDHB | CYP2W1 | 0.27 | FG.Mac.C1QB.SEPP1 | C1QB | 3.49 |
| FG.B/DZ.TOP2A.UBE2C | SKA1 | 0.31 | FG.EC.OLFM4.MARCKSL1 | CYP2W1 | 0.29 | FG.Mac.C1QB.SEPP1 | C1QC | 3.22 |
| FG.B/DZ.TOP2A.UBE2C | GNAI2 | 0.31 | FG.EC.OLFM4.MARCKSL1 | CA9 | 0.52 | FG.Mac.C1QB.SEPP1 | SEPP1 | 3.08 |
| FG.B/DZ.TOP2A.UBE2C | TMED8 | 0.31 | FG.EC.OLFM4.SLC12A2 | CA9 | 0.50 | FG.Mac.C1QB.SEPP1 | CST3 | 3.07 |
| FG.B/DZ.TOP2A.UBE2C | TCF19 | 0.31 | FG.EC.OLFM4.SLC12A2 | PPP1R1B | 1.08 | FG.Mac.C1QB.SEPP1 | TYROBP | 2.87 |
| FG.B/DZ.TOP2A.UBE2C | DCLRE1C | 0.31 | FG.EC.RBP2.ALDOB | RBP2 | 2.85 | FG.Mac.C1QB.SEPP1 | AIF1 | 2.79 |
| FG.B/DZ.TOP2A.UBE2C | FGD6 | 0.30 | FG.EC.RBP2.ALDOB | ALDOB | 2.58 | FG.Mac.C1QB.SEPP1 | SLC40A1 | 2.44 |
| FG.B/DZ.TOP2A.UBE2C | ZWILCH | 0.30 | FG.EC.RBP2.ALDOB | PIGR | 2.47 | FG.Mac.C1QB.SEPP1 | MS4A6A | 2.43 |
| FG.B/DZ.TOP2A.UBE2C | C21orf58 | 0.30 | FG.EC.RBP2.ALDOB | FABP1 | 2.45 | FG.Mac.C1QB.SEPP1 | HLA-DPA1 | 2.40 |
| FG.B/DZ.TOP2A.UBE2C | OXCT1 | 0.29 | FG.EC.RBP2.ALDOB | PRAP1 | 2.31 | FG.Mac.C1QB.SEPP1 | FCER1G | 2.35 |
| FG.B/DZ.TOP2A.UBE2C | PLEKHA2 | 0.29 | FG.EC.RBP2.ALDOB | APOA4 | 2.15 | FG.Mac.C1QB.SEPP1 | NPC2 | 2.29 |
| FG.B/DZ.TOP2A.UBE2C | KATNAL1 | 0.28 | FG.EC.RBP2.ALDOB | ANPEP | 2.15 | FG.Mac.C1QB.SEPP1 | DNASE1L3 | 2.29 |
| FG.B/DZ.TOP2A.UBE2C | ARHGEF39 | 0.28 | FG.EC.RBP2.ALDOB | SMIM24 | 2.14 | FG.Mac.C1QB.SEPP1 | FTL | 2.28 |
| FG.B/DZ.TOP2A.UBE2C | IL4R | 0.28 | FG.EC.RBP2.ALDOB | ADIRF | 2.12 | FG.Mac.C1QB.SEPP1 | LYZ | 2.26 |
| FG.B/DZ.TOP2A.UBE2C | E2F8 | 0.28 | FG.EC.RBP2.ALDOB | FABP2 | 2.11 | FG.Mac.C1QB.SEPP1 | CCL3 | 2.14 |
| FG.B/DZ.TOP2A.UBE2C | PA2G4 | 0.57 | FG.EC.RBP2.ALDOB | PCK1 | 2.01 | FG.Mac.C1QB.SEPP1 | CPVL | 2.13 |
| FG.B/DZ.TOP2A.UBE2C | IMMP1L | 0.33 | FG.EC.RBP2.ALDOB | MT-ND4L | 1.94 | FG.Mac.C1QB.SEPP1 | HLA-DRB1 | 2.11 |
| FG.B/DZ.TOP2A.UBE2C | RCSD1 | 0.35 | FG.EC.RBP2.ALDOB | PHGR1 | 1.93 | FG.Mac.C1QB.SEPP1 | HLA-DPB1 | 2.08 |
| FG.B/DZ.TOP2A.UBE2C | IMPDH2 | 0.49 | FG.EC.RBP2.ALDOB | MT-ND5 | 1.93 | FG.Mac.C1QB.SEPP1 | HLA-DRA | 2.06 |
| FG.B/DZ.TOP2A.UBE2C | SRSF4 | 0.38 | FG.EC.RBP2.ALDOB | C19orf33 | 1.90 | FG.Mac.C1QB.SEPP1 | MS4A7 | 2.02 |
| FG.B/DZ.TOP2A.UBE2C | NAP1L4 | 0.32 | FG.EC.RBP2.ALDOB | CDHR5 | 1.88 | FG.Mac.C1QB.SEPP1 | HLA-DRB5 | 2.00 |
| FG.B/DZ.TOP2A.UBE2C | SNRNP40 | 0.44 | FG.EC.RBP2.ALDOB | APOA1 | 1.88 | FG.Mac.C1QB.SEPP1 | IL1B | 1.98 |
| FG.B/DZ.TOP2A.UBE2C | MAP4K2 | 0.28 | FG.EC.RBP2.ALDOB | ELF3 | 1.86 | FG.Mac.C1QB.SEPP1 | FCGRT | 1.91 |
| FG.B/DZ.TOP2A.UBE2C | ZBTB8OS | 0.51 | FG.EC.RBP2.ALDOB | GSTA1 | 1.83 | FG.Mac.C1QB.SEPP1 | FGL2 | 1.87 |
| FG.B/DZ.TOP2A.UBE2C | POLH | 0.35 | FG.EC.RBP2.ALDOB | CLDN4 | 1.81 | FG.Mac.C1QB.SEPP1 | CXCL8 | 1.78 |
| FG.B/DZ.TOP2A.UBE2C | A4GALT | 0.29 | FG.EC.RBP2.ALDOB | MT-CO1 | 1.79 | FG.Mac.C1QB.SEPP1 | CCL3L3 | 1.77 |
| FG.B/DZ.TOP2A.UBE2C | PSMD8 | 0.46 | FG.EC.RBP2.ALDOB | MT-CO3 | 1.79 | FG.Mac.C1QB.SEPP1 | CCL4L2 | 1.76 |
| FG.B/DZ.TOP2A.UBE2C | MTPN | 0.34 | FG.EC.RBP2.ALDOB | SI | 1.77 | FG.Mac.C1QB.SEPP1 | PSAP | 1.74 |
| FG.B/DZ.TOP2A.UBE2C | MRPL27 | 0.45 | FG.EC.RBP2.ALDOB | KRT8 | 1.74 | FG.Mac.C1QB.SEPP1 | LST1 | 1.73 |
| FG.B/DZ.TOP2A.UBE2C | TUBA4A | 0.40 | FG.EC.RBP2.ALDOB | MT-ND1 | 1.70 | FG.Mac.C1QB.SEPP1 | HLA-DMA | 1.72 |
| FG.B/DZ.TOP2A.UBE2C | SLTM | 0.39 | FG.EC.RBP2.ALDOB | TMPRSS15 | 1.69 | FG.Mac.C1QB.SEPP1 | HLA-DQA1 | 1.71 |
| FG.B/DZ.TOP2A.UBE2C | NME1 | 0.63 | FG.EC.RBP2.ALDOB | CES2 | 1.69 | FG.Mac.C1QB.SEPP1 | CD14 | 1.70 |
| FG.B/DZ.TOP2A.UBE2C | FAM192A | 0.40 | FG.EC.RBP2.ALDOB | APOB | 1.69 | FG.Mac.C1QB.SEPP1 | RNASE6 | 1.70 |
| FG.B/DZ.TOP2A.UBE2C | MFHAS1 | 0.29 | FG.EC.RBP2.ALDOB | SLC5A1 | 1.68 | FG.Mac.C1QB.SEPP1 | CD68 | 1.69 |
| FG.B/DZ.TOP2A.UBE2C | TERF2IP | 0.43 | FG.EC.RBP2.ALDOB | MT-CYB | 1.66 | FG.Mac.C1QB.SEPP1 | LGMN | 1.67 |
| FG.B/DZ.TOP2A.UBE2C | VDAC1 | 0.53 | FG.EC.RBP2.ALDOB | AMN | 1.64 | FG.Mac.C1QB.SEPP1 | JAML | 1.67 |
| FG.B/DZ.TOP2A.UBE2C | DDB2 | 0.28 | FG.EC.RBP2.ALDOB | PLCG2 | 1.64 | FG.Mac.C1QB.SEPP1 | SAT1 | 1.65 |
| FG.B/DZ.TOP2A.UBE2C | CFAP20 | 0.32 | FG.EC.RBP2.ALDOB | AKR7A3 | 1.62 | FG.Mac.C1QB.SEPP1 | CTSS | 1.63 |
| FG.B/DZ.TOP2A.UBE2C | WDR34 | 0.38 | FG.EC.RBP2.ALDOB | MT-ND2 | 1.61 | FG.Mac.C1QB.SEPP1 | CD74 | 1.62 |
| FG.B/DZ.TOP2A.UBE2C | DTX1 | 0.25 | FG.EC.RBP2.ALDOB | MT-ND3 | 1.61 | FG.Mac.C1QB.SEPP1 | CTSC | 1.62 |
| FG.B/DZ.TOP2A.UBE2C | HSPD1 | 0.63 | FG.EC.RBP2.ALDOB | MT-ND4 | 1.58 | FG.Mac.C1QB.SEPP1 | GRN | 1.61 |
| FG.B/DZ.TOP2A.UBE2C | KIF18A | 0.32 | FG.EC.RBP2.ALDOB | MT-CO2 | 1.56 | FG.Mac.C1QB.SEPP1 | CTSB | 1.59 |
| FG.B/DZ.TOP2A.UBE2C | BRCA2 | 0.34 | FG.EC.RBP2.ALDOB | S100A14 | 1.53 | FG.Mac.C1QB.SEPP1 | MS4A4A | 1.57 |
| FG.B/DZ.TOP2A.UBE2C | PAFAH1B3 | 0.37 | FG.EC.RBP2.ALDOB | SLC26A3 | 1.51 | FG.Mac.C1QB.SEPP1 | CXCL2 | 1.55 |
| FG.B/DZ.TOP2A.UBE2C | LYN | 0.36 | FG.EC.RBP2.ALDOB | CBR1 | 1.49 | FG.Mac.C1QB.SEPP1 | RNASET2 | 1.55 |
| FG.B/DZ.TOP2A.UBE2C | AKR1B1 | 0.36 | FG.EC.RBP2.ALDOB | TM4SF5 | 1.47 | FG.Mac.C1QB.SEPP1 | VSIG4 | 1.53 |
| FG.B/DZ.TOP2A.UBE2C | BUD31 | 0.42 | FG.EC.RBP2.ALDOB | MT-ATP6 | 1.46 | FG.Mac.C1QB.SEPP1 | HLA-DQB1 | 1.52 |
| FG.B/DZ.TOP2A.UBE2C | PPP1R7 | 0.40 | FG.EC.RBP2.ALDOB | CLDN3 | 1.46 | FG.Mac.C1QB.SEPP1 | GPX1 | 1.50 |
| FG.B/DZ.TOP2A.UBE2C | PPP1R35 | 0.31 | FG.EC.RBP2.ALDOB | KRT19 | 1.46 | FG.Mac.C1QB.SEPP1 | HES1 | 1.49 |
| FG.B/DZ.TOP2A.UBE2C | PRKD3 | 0.32 | FG.EC.RBP2.ALDOB | PTGR1 | 1.45 | FG.Mac.C1QB.SEPP1 | IGSF6 | 1.48 |
| FG.B/DZ.TOP2A.UBE2C | XRCC6 | 0.49 | FG.EC.RBP2.ALDOB | KHK | 1.45 | FG.Mac.C1QB.SEPP1 | S100A11 | 1.45 |
| FG.B/DZ.TOP2A.UBE2C | CD2BP2 | 0.34 | FG.EC.RBP2.ALDOB | CYP3A4 | 1.44 | FG.Mac.C1QB.SEPP1 | LIPA | 1.45 |
| FG.B/DZ.TOP2A.UBE2C | RPAP3 | 0.35 | FG.EC.RBP2.ALDOB | LIPH | 1.43 | FG.Mac.C1QB.SEPP1 | FOLR2 | 1.44 |
| FG.B/DZ.TOP2A.UBE2C | MRPL22 | 0.39 | FG.EC.RBP2.ALDOB | DNASE1 | 1.42 | FG.Mac.C1QB.SEPP1 | CLEC10A | 1.44 |
| FG.B/DZ.TOP2A.UBE2C | TACC1 | 0.28 | FG.EC.RBP2.ALDOB | DGAT1 | 1.41 | FG.Mac.C1QB.SEPP1 | CSF1R | 1.43 |
| FG.B/DZ.TOP2A.UBE2C | TIPIN | 0.32 | FG.EC.RBP2.ALDOB | SULT1A1 | 1.40 | FG.Mac.C1QB.SEPP1 | FAM26F | 1.39 |
| FG.B/DZ.TOP2A.UBE2C | CCT4 | 0.47 | FG.EC.RBP2.ALDOB | LGALS4 | 1.39 | FG.Mac.C1QB.SEPP1 | ITM2B | 1.38 |
| FG.B/DZ.TOP2A.UBE2C | HSPA4 | 0.36 | FG.EC.RBP2.ALDOB | CYP3A5 | 1.37 | FG.Mac.C1QB.SEPP1 | C1orf54 | 1.38 |
| FG.B/DZ.TOP2A.UBE2C | VGLL4 | 0.31 | FG.EC.RBP2.ALDOB | PPP1R16A | 1.37 | FG.Mac.C1QB.SEPP1 | AXL | 1.38 |
| FG.B/DZ.TOP2A.UBE2C | CCT7 | 0.40 | FG.EC.RBP2.ALDOB | MT1G | 1.37 | FG.Mac.C1QB.SEPP1 | MPEG1 | 1.35 |
| FG.B/DZ.TOP2A.UBE2C | ATP5B | 0.56 | FG.EC.RBP2.ALDOB | PCK2 | 1.37 | FG.Mac.C1QB.SEPP1 | PLD3 | 1.34 |
| FG.B/DZ.TOP2A.UBE2C | NCAPG2 | 0.32 | FG.EC.RBP2.ALDOB | MME | 1.37 | FG.Mac.C1QB.SEPP1 | CD63 | 1.31 |
| FG.B/DZ.TOP2A.UBE2C | PRPF38A | 0.29 | FG.EC.RBP2.ALDOB | ATP5D | 1.35 | FG.Mac.C1QB.SEPP1 | CXCL3 | 1.30 |
| FG.B/DZ.TOP2A.UBE2C | TMEM106C | 0.43 | FG.EC.RBP2.ALDOB | KRT18 | 1.34 | FG.Mac.C1QB.SEPP1 | STAB1 | 1.30 |
| FG.B/DZ.TOP2A.UBE2C | SEL1L3 | 0.42 | FG.EC.RBP2.ALDOB | REEP6 | 1.29 | FG.Mac.C1QB.SEPP1 | PLAUR | 1.29 |
| FG.B/DZ.TOP2A.UBE2C | TIAL1 | 0.34 | FG.EC.RBP2.ALDOB | SMIM22 | 1.26 | FG.Mac.C1QB.SEPP1 | CYBA | 1.28 |
| FG.B/DZ.TOP2A.UBE2C | POLR2D | 0.28 | FG.EC.RBP2.ALDOB | CLDN15 | 1.26 | FG.Mac.C1QB.SEPP1 | GLUL | 1.25 |
| FG.B/LZ.CD74.CD83 | MARCKSL1 | 2.02 | FG.EC.RBP2.ALDOB | CYB5A | 1.26 | FG.Mac.C1QB.SEPP1 | VMO1 | 1.22 |
| FG.B/LZ.CD74.CD83 | RGS13 | 1.97 | FG.EC.RBP2.ALDOB | OAT | 1.25 | FG.Mac.C1QB.SEPP1 | GPR34 | 1.18 |
| FG.B/LZ.CD74.CD83 | MS4A1 | 1.54 | FG.EC.RBP2.ALDOB | MUC13 | 1.25 | FG.Mac.C1QB.SEPP1 | MRC1 | 1.16 |
| FG.B/LZ.CD74.CD83 | SMIM14 | 1.48 | FG.EC.RBP2.ALDOB | EPCAM | 1.25 | FG.Mac.C1QB.SEPP1 | RNF130 | 1.14 |
| FG.B/LZ.CD74.CD83 | SERPINA9 | 1.43 | FG.EC.RBP2.ALDOB | S100A6 | 1.24 | FG.Mac.C1QB.SEPP1 | TGFBI | 1.14 |
| FG.B/LZ.CD74.CD83 | BASP1 | 1.40 | FG.EC.RBP2.ALDOB | MYO1A | 1.24 | FG.Mac.C1QB.SEPP1 | TMEM176B | 1.14 |
| FG.B/LZ.CD74.CD83 | LRMP | 1.37 | FG.EC.RBP2.ALDOB | CYSTM1 | 1.23 | FG.Mac.C1QB.SEPP1 | FTH1 | 1.10 |
| FG.B/LZ.CD74.CD83 | CD79B | 1.25 | FG.EC.RBP2.ALDOB | MTTP | 1.22 | FG.Mac.C1QB.SEPP1 | IGF1 | 1.07 |
| FG.B/LZ.CD74.CD83 | BIK | 1.22 | FG.EC.RBP2.ALDOB | CLDN7 | 1.20 | FG.Mac.C1QB.SEPP1 | CLEC7A | 1.05 |
| FG.B/LZ.CD74.CD83 | GMDS | 1.19 | FG.EC.RBP2.ALDOB | TSPAN8 | 1.19 | FG.Mac.C1QB.SEPP1 | PTGS2 | 1.03 |
| FG.B/LZ.CD74.CD83 | HMGA1 | 1.15 | FG.EC.RBP2.ALDOB | AKR1B10 | 1.18 | FG.Mac.C1QB.SEPP1 | CCL4 | 1.03 |
| FG.B/LZ.CD74.CD83 | ARPC2 | 1.10 | FG.EC.RBP2.ALDOB | APOC3 | 1.18 | FG.Mac.C1QB.SEPP1 | DAB2 | 1.02 |
| FG.B/LZ.CD74.CD83 | CD40 | 1.10 | FG.EC.RBP2.ALDOB | NEAT1 | 1.18 | FG.Mac.C1QB.SEPP1 | CD36 | 1.01 |
| FG.B/LZ.CD74.CD83 | VPREB3 | 1.08 | FG.EC.RBP2.ALDOB | HSD17B2 | 1.17 | FG.Mac.C1QB.SEPP1 | LPAR6 | 0.98 |
| FG.B/LZ.CD74.CD83 | LMO2 | 1.08 | FG.EC.RBP2.ALDOB | SLC39A5 | 1.17 | FG.Mac.C1QB.SEPP1 | FPR3 | 0.96 |
| FG.B/LZ.CD74.CD83 | HLA-DMA | 1.08 | FG.EC.RBP2.ALDOB | DHRS11 | 1.16 | FG.Mac.C1QB.SEPP1 | TMSB4X | 0.96 |
| FG.B/LZ.CD74.CD83 | ACTG1 | 1.07 | FG.EC.RBP2.ALDOB | SAT2 | 1.16 | FG.Mac.C1QB.SEPP1 | CD209 | 0.94 |
| FG.B/LZ.CD74.CD83 | AC079767.4 | 1.05 | FG.EC.RBP2.ALDOB | HSPB1 | 1.16 | FG.Mac.C1QB.SEPP1 | MNDA | 0.94 |
| FG.B/LZ.CD74.CD83 | HLA-DMB | 1.04 | FG.EC.RBP2.ALDOB | MISP | 1.16 | FG.Mac.C1QB.SEPP1 | AOAH | 0.94 |
| FG.B/LZ.CD74.CD83 | RGS16 | 1.04 | FG.EC.RBP2.ALDOB | FBP1 | 1.15 | FG.Mac.C1QB.SEPP1 | HCK | 0.89 |
| FG.B/LZ.CD74.CD83 | FCRLA | 1.04 | FG.EC.RBP2.ALDOB | DSP | 1.15 | FG.Mac.C1QB.SEPP1 | LILRB5 | 0.85 |
| FG.B/LZ.CD74.CD83 | CD79A | 1.02 | FG.EC.RBP2.ALDOB | MPST | 1.15 | FG.Mac.C1QB.SEPP1 | CD163 | 0.82 |
| FG.B/LZ.CD74.CD83 | CD22 | 1.02 | FG.EC.RBP2.ALDOB | FUOM | 1.14 | FG.Mac.C1QB.SEPP1 | C3AR1 | 0.81 |
| FG.B/LZ.CD74.CD83 | TCL1A | 1.02 | FG.EC.RBP2.ALDOB | FAM46A | 1.13 | FG.Mac.C1QB.SEPP1 | CD163L1 | 0.74 |
| FG.B/LZ.CD74.CD83 | POU2F2 | 1.01 | FG.EC.RBP2.ALDOB | PRSS3 | 1.13 | FG.Mac.C1QB.SEPP1 | TBXAS1 | 0.87 |
| FG.B/LZ.CD74.CD83 | RFTN1 | 1.01 | FG.EC.RBP2.ALDOB | MTRNR2L8 | 1.12 | FG.Mac.C1QB.SEPP1 | P2RY13 | 0.78 |
| FG.B/LZ.CD74.CD83 | PARP1 | 1.00 | FG.EC.RBP2.ALDOB | CMBL | 1.11 | FG.Mac.CCL3.HES1 | CCL3 | 3.89 |
| FG.B/LZ.CD74.CD83 | CAMK1 | 1.00 | FG.EC.RBP2.ALDOB | COX5B | 1.11 | FG.Mac.CCL3.HES1 | CCL3L3 | 3.67 |
| FG.B/LZ.CD74.CD83 | SWAP70 | 1.00 | FG.EC.RBP2.ALDOB | GATM | 1.11 | FG.Mac.CCL3.HES1 | CCL4L2 | 3.28 |
| FG.B/LZ.CD74.CD83 | RP11-231C14.7 | 0.98 | FG.EC.RBP2.ALDOB | SLC25A37 | 1.10 | FG.Mac.CCL3.HES1 | C1QA | 3.17 |
| FG.B/LZ.CD74.CD83 | HLA-DQA1 | 0.96 | FG.EC.RBP2.ALDOB | EDF1 | 1.10 | FG.Mac.CCL3.HES1 | CXCL8 | 3.14 |
| FG.B/LZ.CD74.CD83 | LAT2 | 0.96 | FG.EC.RBP2.ALDOB | RP11-467L13.7 | 1.10 | FG.Mac.CCL3.HES1 | C1QB | 2.98 |
| FG.B/LZ.CD74.CD83 | AC023590.1 | 0.95 | FG.EC.RBP2.ALDOB | EFNA1 | 1.09 | FG.Mac.CCL3.HES1 | IL1B | 2.94 |
| FG.B/LZ.CD74.CD83 | SPIB | 0.95 | FG.EC.RBP2.ALDOB | ALPI | 1.08 | FG.Mac.CCL3.HES1 | C1QC | 2.94 |
| FG.B/LZ.CD74.CD83 | BCAS4 | 0.91 | FG.EC.RBP2.ALDOB | ACE | 1.08 | FG.Mac.CCL3.HES1 | CXCL2 | 2.89 |
| FG.B/LZ.CD74.CD83 | PRPSAP2 | 0.87 | FG.EC.RBP2.ALDOB | C11orf86 | 1.08 | FG.Mac.CCL3.HES1 | CXCL3 | 2.82 |
| FG.B/LZ.CD74.CD83 | RRAS2 | 0.86 | FG.EC.RBP2.ALDOB | ATF3 | 1.07 | FG.Mac.CCL3.HES1 | CCL4 | 2.74 |
| FG.B/LZ.CD74.CD83 | P2RX5 | 0.86 | FG.EC.RBP2.ALDOB | CA2 | 1.06 | FG.Mac.CCL3.HES1 | SEPP1 | 2.63 |
| FG.B/LZ.CD74.CD83 | MTMR14 | 0.83 | FG.EC.RBP2.ALDOB | PEBP1 | 1.06 | FG.Mac.CCL3.HES1 | TYROBP | 2.49 |
| FG.B/LZ.CD74.CD83 | TNFRSF13C | 0.82 | FG.EC.RBP2.ALDOB | AGPAT2 | 1.05 | FG.Mac.CCL3.HES1 | AIF1 | 2.37 |
| FG.B/LZ.CD74.CD83 | KLHL6 | 0.81 | FG.EC.RBP2.ALDOB | MXD1 | 1.05 | FG.Mac.CCL3.HES1 | MS4A6A | 2.18 |
| FG.B/LZ.CD74.CD83 | DHRS9 | 0.77 | FG.EC.RBP2.ALDOB | SLC6A19 | 1.04 | FG.Mac.CCL3.HES1 | IER3 | 2.15 |
| FG.B/LZ.CD74.CD83 | HTR3A | 0.72 | FG.EC.RBP2.ALDOB | TTC36 | 1.04 | FG.Mac.CCL3.HES1 | FCER1G | 2.14 |
| FG.B/LZ.CD74.CD83 | STAG3 | 0.72 | FG.EC.RBP2.ALDOB | MIR194-2HG | 1.03 | FG.Mac.CCL3.HES1 | MS4A7 | 2.09 |
| FG.B/LZ.CD74.CD83 | VNN2 | 0.70 | FG.EC.RBP2.ALDOB | CREB3L3 | 1.03 | FG.Mac.CCL3.HES1 | SLC40A1 | 2.00 |
| FG.B/LZ.CD74.CD83 | NEIL1 | 0.79 | FG.EC.RBP2.ALDOB | MUC3A | 1.03 | FG.Mac.CCL3.HES1 | DNASE1L3 | 1.99 |
| FG.B/LZ.CD74.CD83 | HLA-DQB1 | 1.05 | FG.EC.RBP2.ALDOB | CCL25 | 1.02 | FG.Mac.CCL3.HES1 | CPVL | 1.85 |
| FG.B/LZ.CD74.CD83 | GPR18 | 0.86 | FG.EC.RBP2.ALDOB | ZG16 | 1.02 | FG.Mac.CCL3.HES1 | PLAUR | 1.84 |
| FG.B/LZ.CD74.CD83 | BCL2A1 | 0.89 | FG.EC.RBP2.ALDOB | VIL1 | 1.01 | FG.Mac.CCL3.HES1 | SOD2 | 1.81 |
| FG.B/LZ.CD74.CD83 | LCP1 | 0.83 | FG.EC.RBP2.ALDOB | HBEGF | 1.00 | FG.Mac.CCL3.HES1 | CD14 | 1.78 |
| FG.B/LZ.CD74.CD83 | HOPX | 1.04 | FG.EC.RBP2.ALDOB | ADA | 1.00 | FG.Mac.CCL3.HES1 | FGL2 | 1.76 |
| FG.B/LZ.CD74.CD83 | HMCES | 1.07 | FG.EC.RBP2.ALDOB | KRT20 | 1.00 | FG.Mac.CCL3.HES1 | LYZ | 1.75 |
| FG.B/LZ.CD74.CD83 | CTSH | 0.90 | FG.EC.RBP2.ALDOB | CDH17 | 1.00 | FG.Mac.CCL3.HES1 | PTGS2 | 1.74 |
| FG.B/LZ.CD74.CD83 | ANP32B | 1.06 | FG.EC.RBP2.ALDOB | ALKBH7 | 0.99 | FG.Mac.CCL3.HES1 | TNF | 1.70 |
| FG.B/LZ.CD74.CD83 | EBI3 | 0.58 | FG.EC.RBP2.ALDOB | CISD3 | 0.99 | FG.Mac.CCL3.HES1 | JAML | 1.61 |
| FG.cDC1.CLEC9A.XCR1 | CPVL | 2.87 | FG.EC.RBP2.ALDOB | LGALS3 | 0.97 | FG.Mac.CCL3.HES1 | LST1 | 1.54 |
| FG.cDC1.CLEC9A.XCR1 | C1orf54 | 2.36 | FG.EC.RBP2.ALDOB | CDHR2 | 0.97 | FG.Mac.CCL3.HES1 | CSF1R | 1.48 |
| FG.cDC1.CLEC9A.XCR1 | LGALS2 | 2.30 | FG.EC.RBP2.ALDOB | ACADVL | 0.96 | FG.Mac.CCL3.HES1 | ICAM1 | 1.47 |
| FG.cDC1.CLEC9A.XCR1 | IDO1 | 2.28 | FG.EC.RBP2.ALDOB | ACSL5 | 0.96 | FG.Mac.CCL3.HES1 | VSIG4 | 1.46 |
| FG.cDC1.CLEC9A.XCR1 | DNASE1L3 | 2.25 | FG.EC.RBP2.ALDOB | TKFC | 0.95 | FG.Mac.CCL3.HES1 | AXL | 1.41 |
| FG.cDC1.CLEC9A.XCR1 | LYZ | 2.24 | FG.EC.RBP2.ALDOB | AOC1 | 0.95 | FG.Mac.CCL3.HES1 | MPEG1 | 1.39 |
| FG.cDC1.CLEC9A.XCR1 | CLEC9A | 1.94 | FG.EC.RBP2.ALDOB | ATP1A1 | 0.95 | FG.Mac.CCL3.HES1 | CD68 | 1.39 |
| FG.cDC1.CLEC9A.XCR1 | PPT1 | 1.73 | FG.EC.RBP2.ALDOB | P4HB | 0.93 | FG.Mac.CCL3.HES1 | RNASE6 | 1.39 |
| FG.cDC1.CLEC9A.XCR1 | SGK1 | 1.69 | FG.EC.RBP2.ALDOB | SLC4A7 | 0.93 | FG.Mac.CCL3.HES1 | CLEC10A | 1.39 |
| FG.cDC1.CLEC9A.XCR1 | CPNE3 | 1.69 | FG.EC.RBP2.ALDOB | MTRNR2L12 | 0.93 | FG.Mac.CCL3.HES1 | MAFB | 1.38 |
| FG.cDC1.CLEC9A.XCR1 | AIF1 | 1.67 | FG.EC.RBP2.ALDOB | MALL | 0.93 | FG.Mac.CCL3.HES1 | MS4A4A | 1.38 |
| FG.cDC1.CLEC9A.XCR1 | PTPRE | 1.62 | FG.EC.RBP2.ALDOB | TM4SF4 | 0.93 | FG.Mac.CCL3.HES1 | STAB1 | 1.33 |
| FG.cDC1.CLEC9A.XCR1 | PLEK | 1.48 | FG.EC.RBP2.ALDOB | MT2A | 0.93 | FG.Mac.CCL3.HES1 | FOLR2 | 1.32 |
| FG.cDC1.CLEC9A.XCR1 | MPEG1 | 1.47 | FG.EC.RBP2.ALDOB | CHP2 | 0.93 | FG.Mac.CCL3.HES1 | FAM26F | 1.29 |
| FG.cDC1.CLEC9A.XCR1 | FGL2 | 1.46 | FG.EC.RBP2.ALDOB | COX4I1 | 0.93 | FG.Mac.CCL3.HES1 | CLEC7A | 1.23 |
| FG.cDC1.CLEC9A.XCR1 | ASB2 | 1.35 | FG.EC.RBP2.ALDOB | LCT | 0.92 | FG.Mac.CCL3.HES1 | TGFBI | 1.23 |
| FG.cDC1.CLEC9A.XCR1 | SPI1 | 1.35 | FG.EC.RBP2.ALDOB | SLC13A2 | 0.90 | FG.Mac.CCL3.HES1 | C1orf54 | 1.20 |
| FG.cDC1.CLEC9A.XCR1 | CADM1 | 1.28 | FG.EC.RBP2.ALDOB | FLJ22763 | 0.89 | FG.Mac.CCL3.HES1 | IGSF6 | 1.17 |
| FG.cDC1.CLEC9A.XCR1 | RAB32 | 1.28 | FG.EC.RBP2.ALDOB | SULT1A2 | 0.89 | FG.Mac.CCL3.HES1 | MRC1 | 1.12 |
| FG.cDC1.CLEC9A.XCR1 | RP11-1143G9.4 | 1.23 | FG.EC.RBP2.ALDOB | HOOK2 | 0.89 | FG.Mac.CCL3.HES1 | LILRB5 | 1.12 |
| FG.cDC1.CLEC9A.XCR1 | MNDA | 1.20 | FG.EC.RBP2.ALDOB | MT-ATP8 | 0.89 | FG.Mac.CCL3.HES1 | LPAR6 | 1.07 |
| FG.cDC1.CLEC9A.XCR1 | CCND1 | 1.17 | FG.EC.RBP2.ALDOB | SLC15A1 | 0.89 | FG.Mac.CCL3.HES1 | CD209 | 1.02 |
| FG.cDC1.CLEC9A.XCR1 | SHTN1 | 1.16 | FG.EC.RBP2.ALDOB | GNA11 | 0.88 | FG.Mac.CCL3.HES1 | FPR3 | 0.97 |
| FG.cDC1.CLEC9A.XCR1 | FNIP2 | 1.13 | FG.EC.RBP2.ALDOB | S100G | 0.88 | FG.Mac.CCL3.HES1 | KB-1507C5.4 | 0.97 |
| FG.cDC1.CLEC9A.XCR1 | CLEC7A | 1.04 | FG.EC.RBP2.ALDOB | ANGPTL4 | 0.88 | FG.Mac.CCL3.HES1 | CD163 | 0.97 |
| FG.cDC1.CLEC9A.XCR1 | CLNK | 1.02 | FG.EC.RBP2.ALDOB | ATP5I | 0.88 | FG.Mac.CCL3.HES1 | AOAH | 0.93 |
| FG.cDC1.CLEC9A.XCR1 | VMO1 | 0.98 | FG.EC.RBP2.ALDOB | VMP1 | 0.87 | FG.Mac.CCL3.HES1 | TBXAS1 | 0.93 |
| FG.cDC1.CLEC9A.XCR1 | BATF3 | 0.97 | FG.EC.RBP2.ALDOB | MYH14 | 0.86 | FG.Mac.CCL3.HES1 | TNFAIP2 | 0.92 |
| FG.cDC1.CLEC9A.XCR1 | WDFY4 | 0.97 | FG.EC.RBP2.ALDOB | SLC2A5 | 0.86 | FG.Mac.CCL3.HES1 | CD163L1 | 0.90 |
| FG.cDC1.CLEC9A.XCR1 | XCR1 | 0.95 | FG.EC.RBP2.ALDOB | GPX4 | 0.85 | FG.Mac.CCL3.HES1 | PZRY13 | 0.88 |
| FG.cDC1.CLEC9A.XCR1 | TACSTD2 | 0.95 | FG.EC.RBP2.ALDOB | HEBP1 | 0.85 | FG.Mac.CCL3.HES1 | GPR34 | 0.88 |
| FG.cDC1.CLEC9A.XCR1 | RAB7B | 0.89 | FG.EC.RBP2.ALDOB | ATP1B1 | 0.85 | FG.Mac.CCL3.HES1 | DAB2 | 0.86 |
| FG.cDC1.CLEC9A.XCR1 | HCK | 0.85 | FG.EC.RBP2.ALDOB | ID1 | 0.84 | FG.Mac.CCL3.HES1 | VMO1 | 0.85 |
| FG.cDC1.CLEC9A.XCR1 | SLAMF8 | 0.77 | FG.EC.RBP2.ALDOB | COX6B1 | 0.84 | FG.Mac.CCL3.HES1 | NLRP3 | 0.85 |
| FG.cDC1.CLEC9A.XCR1 | P2RY14 | 0.74 | FG.EC.RBP2.ALDOB | SLC22A18 | 0.84 | FG.Mac.CCL3.HES1 | C3AR1 | 0.82 |
| FG.cDC1.CLEC9A.XCR1 | CSF2RA | 0.69 | FG.EC.RBP2.ALDOB | ACAA1 | 0.84 | FG.Mac.CCL3.HES1 | ADAP2 | 0.81 |
| FG.cDC1.CLEC9A.XCR1 | ENPP1 | 0.68 | FG.EC.RBP2.ALDOB | MIR22HG | 0.84 | FG.Mac.CCL3.HES1 | HCK | 0.76 |
| FG.cDC1.CLEC9A.XCR1 | DBN1 | 0.65 | FG.EC.RBP2.ALDOB | UGT2B17 | 0.83 | FG.Mac.CCL3.HES1 | ADORA3 | 0.48 |
| FG.cDC1.CLEC9A.XCR1 | FLT3 | 0.59 | FG.EC.RBP2.ALDOB | CHCHD10 | 0.83 | FG.Mac.CCL3.HES1 | IGF1 | 0.98 |
| FG.cDC1.CLEC9A.XCR1 | HAVCR2 | 0.54 | FG.EC.RBP2.ALDOB | SLC2A2 | 0.83 | FG.Mac.CCL3.HES1 | MSR1 | 0.71 |
| FG.cDC1.CLEC9A.XCR1 | SERPINF2 | 0.51 | FG.EC.RBP2.ALDOB | NDUFB7 | 0.82 | FG.Mac.CCL3.HES1 | BCL2A1 | 1.71 |
| FG.cDC1.CLEC9A.XCR1 | MYCL | 0.49 | FG.EC.RBP2.ALDOB | PBLD | 0.82 | FG.Mac.CCL3.HES1 | LACC1 | 0.75 |
| FG.cDC1.CLEC9A.XCR1 | SLC24A4 | 0.48 | FG.EC.RBP2.ALDOB | SLC39A4 | 0.82 | FG.Mac.CCL3.HES1 | CTSB | 1.65 |
| FG.cDC1.CLEC9A.XCR1 | RP11-798K3.3 | 0.45 | FG.EC.RBP2.ALDOB | SDCBP2 | 0.82 | FG.Mac.CCL3.HES1 | LILRB2 | 0.50 |
| FG.cDC1.CLEC9A.XCR1 | PPM1J | 0.34 | FG.EC.RBP2.ALDOB | SLC20A1 | 0.82 | FG.Mac.CCL3.HES1 | ADGRE2 | 0.58 |
| FG.cDC1.CLEC9A.XCR1 | C20orf27 | 1.03 | FG.EC.RBP2.ALDOB | FOLH1 | 0.82 | FG.Mac.CCL3.HES1 | CD36 | 0.92 |
| FG.cDC1.CLEC9A.XCR1 | P2RY6 | 0.48 | FG.EC.RBP2.ALDOB | RIOK3 | 0.82 | FG.Mac.CXCL3.CXCL8 | CCL3 | 4.06 |
| FG.cDC1.CLEC9A.XCR1 | FKBP1B | 0.59 | FG.EC.RBP2.ALDOB | ETFB | 0.81 | FG.Mac.CXCL3.CXCL8 | CXCL3 | 3.94 |
| FG.cDC1.CLEC9A.XCR1 | BID | 1.19 | FG.EC.RBP2.ALDOB | COMTD1 | 0.81 | FG.Mac.CXCL3.CXCL8 | CXCL8 | 3.64 |
| FG.cDC1.CLEC9A.XCR1 | CLEC1A | 0.33 | FG.EC.RBP2.ALDOB | SULT1B1 | 0.80 | FG.Mac.CXCL3.CXCL8 | CCL3L3 | 3.50 |
| FG.cDC2.AREG.CD1C | IL1B | 1.92 | FG.EC.RBP2.ALDOB | CRYL1 | 0.80 | FG.Mac.CXCL3.CXCL8 | CCL4L2 | 3.48 |
| FG.cDC2.AREG.CD1C | CLEC10A | 1.89 | FG.EC.RBP2.ALDOB | GDA | 0.80 | FG.Mac.CXCL3.CXCL8 | CXCL2 | 3.40 |
| FG.cDC2.AREG.CD1C | IFI30 | 1.79 | FG.EC.RBP2.ALDOB | MDK | 0.80 | FG.Mac.CXCL3.CXCL8 | PLAUR | 3.38 |
| FG.cDC2.AREG.CD1C | FCER1A | 1.64 | FG.EC.RBP2.ALDOB | BLVRB | 0.80 | FG.Mac.CXCL3.CXCL8 | CCL4 | 3.28 |
| FG.cDC2.AREG.CD1C | MNDA | 1.63 | FG.EC.RBP2.ALDOB | PDK4 | 0.80 | FG.Mac.CXCL3.CXCL8 | IL1B | 3.09 |
| FG.cDC2.AREG.CD1C | IGSF6 | 1.43 | FG.EC.RBP2.ALDOB | MS4A8 | 0.79 | FG.Mac.CXCL3.CXCL8 | C1QC | 2.94 |
| FG.cDC2.AREG.CD1C | CFP | 1.33 | FG.EC.RBP2.ALDOB | NAPRT | 0.79 | FG.Mac.CXCL3.CXCL8 | SEPP1 | 2.70 |
| FG.cDC2.AREG.CD1C | CD207 | 1.03 | FG.EC.RBP2.ALDOB | TM4SF20 | 0.78 | FG.Mac.CXCL3.CXCL8 | C1QA | 2.68 |
| FG.cDC2.AREG.CD1C | CD1E | 1.24 | FG.EC.RBP2.ALDOB | NDUFS6 | 0.78 | FG.Mac.CXCL3.CXCL8 | MS4A6A | 2.66 |
| FG.cDC2.AREG.CD1C | CSF2RA | 1.17 | FG.EC.RBP2.ALDOB | MAOA | 0.78 | FG.Mac.CXCL3.CXCL8 | C1QB | 2.61 |
| FG.cDC2.CD207.FGR | CD207 | 1.70 | FG.EC.RBP2.ALDOB | COX7B | 0.78 | FG.Mac.CXCL3.CXCL8 | SLC40A1 | 2.59 |
| FG.cDC2.CD207.FGR | CFP | 1.56 | FG.EC.RBP2.ALDOB | DDC | 0.77 | FG.Mac.CXCL3.CXCL8 | AIF1 | 2.55 |
| FG.cDC2.CD207.FGR | CD1E | 1.44 | FG.EC.RBP2.ALDOB | ESPN | 0.77 | FG.Mac.CXCL3.CXCL8 | BCL2A1 | 2.48 |
| FG.cDC2.CLEC10A.CD207 | CLEC10A | 2.20 | FG.EC.RBP2.ALDOB | AGR3 | 0.77 | FG.Mac.CXCL3.CXCL8 | SOD2 | 2.42 |
| FG.cDC2.CLEC10A.CD207 | IGSF6 | 1.58 | FG.EC.RBP2.ALDOB | NDUFS7 | 0.77 | FG.Mac.CXCL3.CXCL8 | HLA-DQA2 | 2.34 |
| FG.cDC2.CLEC10A.CD207 | CFP | 1.56 | FG.EC.RBP2.ALDOB | BDH2 | 0.77 | FG.Mac.CXCL3.CXCL8 | FCER1G | 2.33 |
| FG.cDC2.CLEC10A.CD207 | CSF2RA | 1.30 | FG.EC.RBP2.ALDOB | GLS | 0.77 | FG.Mac.CXCL3.CXCL8 | MS4A7 | 2.31 |
| FG.cDC2.CLEC10A.CD207 | CD207 | 1.22 | FG.EC.RBP2.ALDOB | ADH1C | 0.77 | FG.Mac.CXCL3.CXCL8 | IGSF6 | 2.21 |
| FG.cDC2.CLEC10A.CD207 | CSTA | 1.10 | FG.EC.RBP2.ALDOB | ACE2 | 0.77 | FG.Mac.CXCL3.CXCL8 | DNASE1L3 | 2.20 |
| FG.cDC2.CLEC10A.CD207 | MNDA | 1.58 | FG.EC.RBP2.ALDOB | NDUFB10 | 0.76 | FG.Mac.CXCL3.CXCL8 | CPVL | 2.17 |
| FG.DC.CCR7.FSCN1 | IDO1 | 2.65 | FG.EC.RBP2.ALDOB | EPS8L3 | 0.76 | FG.Mac.CXCL3.CXCL8 | FGL2 | 2.13 |
| FG.DC.CCR7.FSCN1 | FSCN1 | 2.46 | FG.EC.RBP2.ALDOB | ANXA13 | 0.76 | FG.Mac.CXCL3.CXCL8 | TYROBP | 2.08 |
| FG.DC.CCR7.FSCN1 | CCL19 | 2.28 | FG.EC.RBP2.ALDOB | ARL14 | 0.76 | FG.Mac.CXCL3.CXCL8 | ICAM1 | 1.83 |
| FG.DC.CCR7.FSCN1 | CD1E | 2.25 | FG.EC.RBP2.ALDOB | TPM1 | 0.75 | FG.Mac.CXCL3.CXCL8 | IER3 | 1.82 |
| FG.DC.CCR7.FSCN1 | CCL22 | 2.24 | FG.EC.RBP2.ALDOB | CLTB | 0.75 | FG.Mac.CXCL3.CXCL8 | GLUL | 1.77 |
| FG.DC.CCR7.FSCN1 | LAMP3 | 2.08 | FG.EC.RBP2.ALDOB | LDLR | 0.75 | FG.Mac.CXCL3.CXCL8 | MNDA | 1.76 |
| FG.DC.CCR7.FSCN1 | TREML1 | 0.78 | FG.EC.RBP2.ALDOB | UGT2A3 | 0.75 | FG.Mac.CXCL3.CXCL8 | MS4A4A | 1.76 |
| FG.DC.CCR7.FSCN1 | CD1B | 1.08 | FG.EC.RBP2.ALDOB | LINC01133 | 0.75 | FG.Mac.CXCL3.CXCL8 | CLEC10A | 1.72 |
| FG.DC.CCR7.FSCN1 | TNFAIP2 | 1.35 | FG.EC.RBP2.ALDOB | MT1X | 0.75 | FG.Mac.CXCL3.CXCL8 | JAML | 1.70 |
| FG.DC.CCR7.FSCN1 | CFP | 1.53 | FG.EC.RBP2.ALDOB | DNPH1 | 0.75 | FG.Mac.CXCL3.CXCL8 | RNASE6 | 1.68 |
| FG.DC.LTB.GSN | CFP | 1.84 | FG.EC.RBP2.ALDOB | ALDH1A1 | 0.74 | FG.Mac.CXCL3.CXCL8 | MRC1 | 1.67 |
| FG.DC.LTB.GSN | DEFA4 | 1.50 | FG.EC.RBP2.ALDOB | CPS1 | 0.74 | FG.Mac.CXCL3.CXCL8 | PTGS2 | 1.62 |
| FG.DC.LTB.GSN | ENHO | 1.25 | FG.EC.RBP2.ALDOB | MGST3 | 0.73 | FG.Mac.CXCL3.CXCL8 | MMP10 | 1.61 |
| FG.DC.LTB.GSN | SFTPD | 1.07 | FG.EC.RBP2.ALDOB | MYO15B | 0.72 | FG.Mac.CXCL3.CXCL8 | CD36 | 1.57 |
| FG.DC.LTB.GSN | NRXN2 | 0.74 | FG.EC.RBP2.ALDOB | CIDEB | 0.72 | FG.Mac.CXCL3.CXCL8 | TNF | 1.57 |
| FG.DC.LTB.GSN | CD300LG | 0.41 | FG.EC.RBP2.ALDOB | MT1E | 0.72 | FG.Mac.CXCL3.CXCL8 | VSIG4 | 1.55 |
| FG.DC.LTB.IL22RA2 | IL22RA2 | 1.87 | FG.EC.RBP2.ALDOB | PLS1 | 0.71 | FG.Mac.CXCL3.CXCL8 | C1orf54 | 1.55 |
| FG.EC.ANXA10.VSIG2 | ANXA10 | 1.39 | FG.EC.RBP2.ALDOB | FIS1 | 0.71 | FG.Mac.CXCL3.CXCL8 | C3AR1 | 1.49 |
| FG.EC.ANXA10.VSIG2 | VSIG2 | 0.82 | FG.EC.RBP2.ALDOB | FCGRT | 0.71 | FG.Mac.CXCL3.CXCL8 | LST1 | 1.48 |
| FG.EC.ANXA10.VSIG2 | PDIA2 | 0.56 | FG.EC.RBP2.ALDOB | EPHX2 | 0.71 | FG.Mac.CXCL3.CXCL8 | LPAR6 | 1.46 |
| FG.EC.ANXA10.VSIG2 | PROM1 | 0.44 | FG.EC.RBP2.ALDOB | FAM3B | 0.70 | FG.Mac.CXCL3.CXCL8 | STX11 | 1.44 |
| FG.EC.ANXA10.VSIG2 | MLPH | 0.43 | FG.EC.RBP2.ALDOB | RNF186 | 0.70 | FG.Mac.CXCL3.CXCL8 | GPR34 | 1.37 |
| FG.EC.ANXA10.VSIG2 | KCNE2 | 0.31 | FG.EC.RBP2.ALDOB | CYBRD1 | 0.70 | FG.Mac.CXCL3.CXCL8 | F13A1 | 1.30 |
| FG.EC.APOC3.APOA1 | APOA1 | 4.01 | FG.EC.RBP2.ALDOB | DPEP1 | 0.69 | FG.Mac.CXCL3.CXCL8 | STAB1 | 1.27 |
| FG.EC.APOC3.APOA1 | APOC3 | 3.98 | FG.EC.RBP2.ALDOB | ABHD11-AS1 | 0.69 | FG.Mac.CXCL3.CXCL8 | FCGR2A | 1.16 |
| FG.EC.APOC3.APOA1 | APOA4 | 3.66 | FG.EC.RBP2.ALDOB | GK | 0.69 | FG.Mac.CXCL3.CXCL8 | C5AR1 | 1.15 |
| FG.EC.APOC3.APOA1 | PRAP1 | 3.19 | FG.EC.RBP2.ALDOB | HADHA | 0.69 | FG.Mac.CXCL3.CXCL8 | TNFAIP2 | 1.13 |
| FG.EC.APOC3.APOA1 | ADIRF | 2.67 | FG.EC.RBP2.ALDOB | ATP5G3 | 0.68 | FG.Mac.CXCL3.CXCL8 | CD163L1 | 1.09 |
| FG.EC.APOC3.APOA1 | PHGR1 | 2.58 | FG.EC.RBP2.ALDOB | SFN | 0.68 | FG.Mac.CXCL3.CXCL8 | FPR3 | 1.08 |
| FG.EC.APOC3.APOA1 | ALDOB | 2.57 | FG.EC.RBP2.ALDOB | TMEM41A | 0.68 | FG.Mac.CXCL3.CXCL8 | CD163 | 1.04 |
| FG.EC.APOC3.APOA1 | MT-ND5 | 2.41 | FG.EC.RBP2.ALDOB | SLC25A1 | 0.68 | FG.Mac.CXCL3.CXCL8 | P2RY13 | 1.01 |
| FG.EC.APOC3.APOA1 | CDHR5 | 2.37 | FG.EC.RBP2.ALDOB | ACOX1 | 0.68 | FG.Mac.CXCL3.CXCL8 | NLRP3 | 1.01 |
| FG.EC.APOC3.APOA1 | SMIM24 | 2.27 | FG.EC.RBP2.ALDOB | ATOX1 | 0.68 | FG.Mac.CXCL3.CXCL8 | LILRB5 | 1.00 |
| FG.EC.APOC3.APOA1 | ANPEP | 2.23 | FG.EC.RBP2.ALDOB | EPS8L2 | 0.68 | FG.Mac.CXCL3.CXCL8 | CSF1R | 0.97 |
| FG.EC.APOC3.APOA1 | MT-CO1 | 2.21 | FG.EC.RBP2.ALDOB | FUT2 | 0.67 | FG.Mac.CXCL3.CXCL8 | HLA-DRB5 | 2.20 |
| FG.EC.APOC3.APOA1 | C19orf33 | 2.18 | FG.EC.RBP2.ALDOB | UQCRQ | 0.67 | FG.Mac.CXCL3.CXCL8 | CST3 | 2.11 |
| FG.EC.APOC3.APOA1 | RBP2 | 2.15 | FG.EC.RBP2.ALDOB | TMEM25 | 0.67 | FG.Mac.CXCL3.CXCL8 | PLEK | 1.83 |
| FG.EC.APOC3.APOA1 | MT-ND4L | 2.14 | FG.EC.RBP2.ALDOB | TREH | 0.67 | FG.Mac.CXCL3.CXCL8 | CD68 | 1.61 |
| FG.EC.APOC3.APOA1 | FABP1 | 2.10 | FG.EC.RBP2.ALDOB | TM6SF2 | 0.67 | FG.Mac.CXCL8.IL1B | CXCL8 | 3.93 |
| FG.EC.APOC3.APOA1 | TMPRSS15 | 1.95 | FG.EC.RBP2.ALDOB | ACTN4 | 0.67 | FG.Mac.CXCL8.IL1B | IL1B | 3.60 |
| FG.EC.APOC3.APOA1 | MT-ND2 | 1.91 | FG.EC.RBP2.ALDOB | CBLC | 0.66 | FG.Mac.CXCL8.IL1B | CXCL2 | 3.17 |
| FG.EC.APOC3.APOA1 | TM4SF5 | 1.90 | FG.EC.RBP2.ALDOB | ETS2 | 0.66 | FG.Mac.CXCL8.IL1B | CCL3 | 2.97 |
| FG.EC.APOC3.APOA1 | MT-ND1 | 1.89 | FG.EC.RBP2.ALDOB | HSPA1B | 0.66 | FG.Mac.CXCL8.IL1B | LYZ | 2.85 |
| FG.EC.APOC3.APOA1 | AMN | 1.89 | FG.EC.RBP2.ALDOB | AKR1C3 | 0.66 | FG.Mac.CXCL8.IL1B | CXCL3 | 2.84 |
| FG.EC.APOC3.APOA1 | MT-CO3 | 1.89 | FG.EC.RBP2.ALDOB | TMC5 | 0.66 | FG.Mac.CXCL8.IL1B | SLC40A1 | 2.62 |
| FG.EC.APOC3.APOA1 | MT-ND3 | 1.88 | FG.EC.RBP2.ALDOB | TMEM150B | 0.65 | FG.Mac.CXCL8.IL1B | C1QA | 2.57 |
| FG.EC.APOC3.APOA1 | MDK | 1.83 | FG.EC.RBP2.ALDOB | EPN1 | 0.65 | FG.Mac.CXCL8.IL1B | C1QB | 2.51 |
| FG.EC.APOC3.APOA1 | APOB | 1.77 | FG.EC.RBP2.ALDOB | UQCRC1 | 0.65 | FG.Mac.CXCL8.IL1B | PLAUR | 2.46 |
| FG.EC.APOC3.APOA1 | PCK1 | 1.76 | FG.EC.RBP2.ALDOB | ACADS | 0.64 | FG.Mac.CXCL8.IL1B | C1QC | 2.45 |
| FG.EC.APOC3.APOA1 | MT-ND4 | 1.76 | FG.EC.RBP2.ALDOB | CTSD | 0.64 | FG.Mac.CXCL8.IL1B | IER3 | 2.38 |
| FG.EC.APOC3.APOA1 | MT-CYB | 1.75 | FG.EC.RBP2.ALDOB | VNN1 | 0.64 | FG.Mac.CXCL8.IL1B | MS4A6A | 2.35 |
| FG.EC.APOC3.APOA1 | S100A6 | 1.75 | FG.EC.RBP2.ALDOB | SLC35G1 | 0.64 | FG.Mac.CXCL8.IL1B | MS4A7 | 2.32 |
| FG.EC.APOC3.APOA1 | PLCG2 | 1.73 | FG.EC.RBP2.ALDOB | HMGCS2 | 0.64 | FG.Mac.CXCL8.IL1B | CPVL | 2.30 |
| FG.EC.APOC3.APOA1 | FABP2 | 1.69 | FG.EC.RBP2.ALDOB | SQSTM1 | 0.63 | FG.Mac.CXCL8.IL1B | FGL2 | 2.26 |
| FG.EC.APOC3.APOA1 | MT-ATP6 | 1.69 | FG.EC.RBP2.ALDOB | SPINT1 | 0.63 | FG.Mac.CXCL8.IL1B | CCL3L3 | 2.25 |
| FG.EC.APOC3.APOA1 | SLC26A3 | 1.59 | FG.EC.RBP2.ALDOB | C6orf222 | 0.63 | FG.Mac.CXCL8.IL1B | CCL4L2 | 2.25 |
| FG.EC.APOC3.APOA1 | MT-CO2 | 1.57 | FG.EC.RBP2.ALDOB | ANXA4 | 0.63 | FG.Mac.CXCL8.IL1B | PTGS2 | 2.11 |
| FG.EC.APOC3.APOA1 | CDHR2 | 1.56 | FG.EC.RBP2.ALDOB | MT1H | 0.63 | FG.Mac.CXCL8.IL1B | TYROBP | 2.07 |
| FG.EC.APOC3.APOA1 | AKR7A3 | 1.53 | FG.EC.RBP2.ALDOB | SLC4A4 | 0.63 | FG.Mac.CXCL8.IL1B | STX11 | 1.95 |
| FG.EC.APOC3.APOA1 | CYSTM1 | 1.51 | FG.EC.RBP2.ALDOB | MYO7B | 0.63 | FG.Mac.CXCL8.IL1B | AIF1 | 1.88 |
| FG.EC.APOC3.APOA1 | SLC5A1 | 1.47 | FG.EC.RBP2.ALDOB | ONECUT2 | 0.63 | FG.Mac.CXCL8.IL1B | MAFB | 1.86 |
| FG.EC.APOC3.APOA1 | PIGR | 1.46 | FG.EC.RBP2.ALDOB | AC004754.3 | 0.62 | FG.Mac.CXCL8.IL1B | TNF | 1.81 |
| FG.EC.APOC3.APOA1 | SERPINA1 | 1.45 | FG.EC.RBP2.ALDOB | TXN | 0.62 | FG.Mac.CXCL8.IL1B | TGFBI | 1.76 |
| FG.EC.APOC3.APOA1 | CYP3A4 | 1.44 | FG.EC.RBP2.ALDOB | CDH1 | 0.62 | FG.Mac.CXCL8.IL1B | CLEC10A | 1.73 |
| FG.EC.APOC3.APOA1 | ALPI | 1.43 | FG.EC.RBP2.ALDOB | RAB17 | 0.62 | FG.Mac.CXCL8.IL1B | VSIG4 | 1.69 |
| FG.EC.APOC3.APOA1 | MTRNR2L8 | 1.42 | FG.EC.RBP2.ALDOB | PLCB3 | 0.62 | FG.Mac.CXCL8.IL1B | CSF1R | 1.58 |
| FG.EC.APOC3.APOA1 | KRT8 | 1.42 | FG.EC.RBP2.ALDOB | AADAC | 0.62 | FG.Mac.CXCL8.IL1B | MRC1 | 1.50 |
| FG.EC.APOC3.APOA1 | S100A14 | 1.41 | FG.EC.RBP2.ALDOB | CDC42BPA | 0.61 | FG.Mac.CXCL8.IL1B | C5AR1 | 1.40 |
| FG.EC.APOC3.APOA1 | ATP5D | 1.41 | FG.EC.RBP2.ALDOB | TST | 0.61 | FG.Mac.CXCL8.IL1B | FPR3 | 1.33 |
| FG.EC.APOC3.APOA1 | ELF3 | 1.40 | FG.EC.RBP2.ALDOB | S100A10 | 0.61 | FG.Mac.CXCL8.IL1B | NLRP3 | 1.31 |
| FG.EC.APOC3.APOA1 | FLJ22763 | 1.39 | FG.EC.RBP2.ALDOB | SULT2A1 | 0.61 | FG.Mac.CXCL8.IL1B | LILRB5 | 1.28 |
| FG.EC.APOC3.APOA1 | REEP6 | 1.39 | FG.EC.RBP2.ALDOB | PPP1R14D | 0.61 | FG.Mac.CXCL8.IL1B | DNASE1L3 | 1.84 |
| FG.EC.APOC3.APOA1 | DGAT1 | 1.36 | FG.EC.RBP2.ALDOB | RETSAT | 0.61 | FG.Mono/cDC2.AREG.IL1R2 | FCER1A | 2.32 |
| FG.EC.APOC3.APOA1 | EDF1 | 1.35 | FG.EC.RBP2.ALDOB | NDRG1 | 0.60 | FG.Mono/cDC2.AREG.IL1R2 | HLA-DQA2 | 2.15 |
| FG.EC.APOC3.APOA1 | CES2 | 1.35 | FG.EC.RBP2.ALDOB | ERICH4 | 0.60 | FG.Mono/cDC2.AREG.IL1R2 | CLEC10A | 2.08 |
| FG.EC.APOC3.APOA1 | SMIM22 | 1.32 | FG.EC.RBP2.ALDOB | CYP4F12 | 0.60 | FG.Mono/cDC2.AREG.IL1R2 | MNDA | 2.06 |
| FG.EC.APOC3.APOA1 | KRT19 | 1.32 | FG.EC.RBP2.ALDOB | PSME2 | 0.59 | FG.Mono/cDC2.AREG.IL1R2 | MS4A6A | 2.04 |
| FG.EC.APOC3.APOA1 | C11orf86 | 1.31 | FG.EC.RBP2.ALDOB | LGALS3BP | 0.59 | FG.Mono/cDC2.AREG.IL1R2 | CPVL | 1.84 |
| FG.EC.APOC3.APOA1 | S100G | 1.31 | FG.EC.RBP2.ALDOB | CYP2C18 | 0.59 | FG.Mono/cDC2.AREG.IL1R2 | CD1E | 1.83 |
| FG.EC.APOC3.APOA1 | SULT1A1 | 1.29 | FG.EC.RBP2.ALDOB | ASS1 | 0.58 | FG.Mono/cDC2.AREG.IL1R2 | IGSF6 | 1.61 |
| FG.EC.APOC3.APOA1 | GPX4 | 1.29 | FG.EC.RBP2.ALDOB | BAIAP2L2 | 0.58 | FG.Mono/cDC2.AREG.IL1R2 | CD207 | 1.36 |
| FG.EC.APOC3.APOA1 | MT-ATP8 | 1.28 | FG.EC.RBP2.ALDOB | GSTA2 | 0.58 | FG.Mono/cDC2.AREG.IL1R2 | CD86 | 1.35 |
| FG.EC.APOC3.APOA1 | ADA | 1.28 | FG.EC.RBP2.ALDOB | MEP1A | 0.58 | FG.Mono/cDC2.AREG.IL1R2 | P2RY13 | 1.27 |
| FG.EC.APOC3.APOA1 | CYP3A5 | 1.27 | FG.EC.RBP2.ALDOB | OTOP3 | 0.58 | FG.Mono/cDC2.AREG.IL1R2 | CFP | 1.23 |
| FG.EC.APOC3.APOA1 | FUOM | 1.26 | FG.EC.RBP2.ALDOB | GPD1 | 0.58 | FG.Mono/cDC2.AREG.IL1R2 | CSF2RA | 1.15 |
| FG.EC.APOC3.APOA1 | CLDN4 | 1.26 | FG.EC.RBP2.ALDOB | FAM132A | 0.58 | FG.Mono/cDC2.AREG.IL1R2 | CLEC4A | 1.08 |
| FG.EC.APOC3.APOA1 | KHK | 1.26 | FG.EC.RBP2.ALDOB | PCSK5 | 0.58 | FG.Mono/cDC2.AREG.IL1R2 | IL1R2 | 1.03 |
| FG.EC.APOC3.APOA1 | MME | 1.25 | FG.EC.RBP2.ALDOB | DHDH | 0.57 | FG.Mono/cDC2.AREG.IL1R2 | MRC1 | 1.02 |
| FG.EC.APOC3.APOA1 | DNASE1 | 1.24 | FG.EC.RBP2.ALDOB | ABCG5 | 0.56 | FG.Mono/cDC2.AREG.IL1R2 | HLA-DQB2 | 0.89 |
| FG.EC.APOC3.APOA1 | MTRNR2L12 | 1.23 | FG.EC.RBP2.ALDOB | DST | 0.56 | FG.Mono/cDC2.AREG.IL1R2 | CACNA2D3 | 0.82 |
| FG.EC.APOC3.APOA1 | SI | 1.23 | FG.EC.RBP2.ALDOB | MTRNR2L1 | 0.56 | FG.Mono/cDC2.AREG.IL1R2 | CD1C | 1.81 |
| FG.EC.APOC3.APOA1 | ENPP7 | 1.21 | FG.EC.RBP2.ALDOB | OSGIN1 | 0.55 | FG.Mono/cDC2.FCER1A.CD1C | FCER1A | 2.40 |
| FG.EC.APOC3.APOA1 | MYO1A | 1.20 | FG.EC.RBP2.ALDOB | XPNPEP2 | 0.55 | FG.Mono/cDC2.FCER1A.CD1C | CST3 | 2.30 |
| FG.EC.APOC3.APOA1 | PCK2 | 1.19 | FG.EC.RBP2.ALDOB | MGAM | 0.54 | FG.Mono/cDC2.FCER1A.CD1C | AIF1 | 2.21 |
| FG.EC.APOC3.APOA1 | AOC1 | 1.19 | FG.EC.RBP2.ALDOB | SLC51B | 0.53 | FG.Mono/cDC2.FCER1A.CD1C | LYZ | 2.11 |
| FG.EC.APOC3.APOA1 | SAT2 | 1.19 | FG.EC.RBP2.ALDOB | FTL | 0.36 | FG.Mono/cDC2.FCER1A.CD1C | MNDA | 2.11 |
| FG.EC.APOC3.APOA1 | MUC3A | 1.18 | FG.EC.RBP2.ALDOB | GOLIM4 | 0.61 | FG.Mono/cDC2.FCER1A.CD1C | LST1 | 2.00 |
| FG.EC.APOC3.APOA1 | PRSS3 | 1.17 | FG.EC.RBP2.ALDOB | PHLDA2 | 0.64 | FG.Mono/cDC2.FCER1A.CD1C | TYROBP | 1.96 |
| FG.EC.APOC3.APOA1 | RP11-160E2.6 | 1.17 | FG.EC.RBP2.ALDOB | KLF3 | 0.59 | FG.Mono/cDC2.FCER1A.CD1C | JAML | 1.96 |
| FG.EC.APOC3.APOA1 | MT1G | 1.15 | FG.EC.RBP2.ALDOB | MGST2 | 0.62 | FG.Mono/cDC2.FCER1A.CD1C | CPVL | 1.92 |
| FG.EC.APOC3.APOA1 | DPEP1 | 1.14 | FG.EC.RBP2.ALDOB | USH1C | 0.60 | FG.Mono/cDC2.FCER1A.CD1C | MS4A6A | 1.92 |
| FG.EC.APOC3.APOA1 | SULT1A2 | 1.13 | FG.EC.RBP2.ALDOB | SERINC2 | 0.61 | FG.Mono/cDC2.FCER1A.CD1C | LGALS2 | 1.85 |
| FG.EC.APOC3.APOA1 | ACE | 1.13 | FG.EC.XIST.OLFM4 | OLFM4 | 2.63 | FG.Mono/cDC2.FCER1A.CD1C | IL1B | 1.82 |
| FG.EC.APOC3.APOA1 | ACAA1 | 1.12 | FG.EC.XIST.OLFM4 | REG1A | 2.28 | FG.Mono/cDC2.FCER1A.CD1C | CLEC10A | 1.79 |
| FG.EC.APOC3.APOA1 | KRT18 | 1.12 | FG.Endth.FOS.FOSB | PLVAP | 3.57 | FG.Mono/cDC2.FCER1A.CD1C | FCER1G | 1.79 |
| FG.EC.APOC3.APOA1 | SLC4A7 | 1.12 | FG.Endth.FOS.FOSB | SLC9A3R2 | 2.67 | FG.Mono/cDC2.FCER1A.CD1C | PLAUR | 1.74 |
| FG.EC.APOC3.APOA1 | CYB5A | 1.12 | FG.Endth.FOS.FOSB | CAV1 | 2.66 | FG.Mono/cDC2.FCER1A.CD1C | IGSF6 | 1.71 |
| FG.EC.APOC3.APOA1 | PPP1R16A | 1.11 | FG.Endth.FOS.FOSB | GNG11 | 2.65 | FG.Mono/cDC2.FCER1A.CD1C | CD1E | 1.51 |
| FG.EC.APOC3.APOA1 | SLC2A5 | 1.10 | FG.Endth.FOS.FOSB | RAMP2 | 2.60 | FG.Mono/cDC2.FCER1A.CD1C | SGK1 | 1.48 |
| FG.EC.APOC3.APOA1 | FBP1 | 1.09 | FG.Endth.FOS.FOSB | CLDN5 | 2.47 | FG.Mono/cDC2.FCER1A.CD1C | CD1C | 1.46 |
| FG.EC.APOC3.APOA1 | SLC15A1 | 1.08 | FG.Endth.FOS.FOSB | TMEM88 | 2.29 | FG.Mono/cDC2.FCER1A.CD1C | SPI1 | 1.29 |
| FG.EC.APOC3.APOA1 | TM4SF20 | 1.08 | FG.Endth.FOS.FOSB | EGFL7 | 2.28 | FG.Mono/cDC2.FCER1A.CD1C | FGL2 | 1.27 |
| FG.EC.APOC3.APOA1 | TM4SF4 | 1.08 | FG.Endth.FOS.FOSB | MGP | 2.08 | FG.Mono/cDC2.FCER1A.CD1C | RNASE6 | 1.27 |
| FG.EC.APOC3.APOA1 | NEAT1 | 1.07 | FG.Endth.FOS.FOSB | ESAM | 2.01 | FG.Mono/cDC2.FCER1A.CD1C | IFI30 | 1.24 |
| FG.EC.APOC3.APOA1 | SLC46A1 | 1.07 | FG.Endth.FOS.FOSB | FLT1 | 1.99 | FG.Mono/cDC2.FCER1A.CD1C | PTPRE | 1.22 |
| FG.EC.APOC3.APOA1 | PPDPF | 1.06 | FG.Endth.FOS.FOSB | RBP7 | 1.95 | FG.Mono/cDC2.FCER1A.CD1C | SAMHD1 | 1.16 |
| FG.EC.APOC3.APOA1 | MUC13 | 1.06 | FG.Endth.FOS.FOSB | SDPR | 1.94 | FG.Mono/cDC2.FCER1A.CD1C | FAM26F | 1.15 |
| FG.EC.APOC3.APOA1 | CREB3L3 | 1.06 | FG.Endth.FOS.FOSB | PCAT19 | 1.85 | FG.Mono/cDC2.FCER1A.CD1C | MPEG1 | 1.14 |
| FG.EC.APOC3.APOA1 | CLDN7 | 1.05 | FG.Endth.FOS.FOSB | RAMP3 | 1.85 | FG.Mono/cDC2.FCER1A.CD1C | PLD4 | 1.12 |
| FG.EC.APOC3.APOA1 | CBR1 | 1.05 | FG.Endth.FOS.FOSB | EMCN | 1.75 | FG.Mono/cDC2.FCER1A.CD1C | CLEC4A | 1.03 |
| FG.EC.APOC3.APOA1 | LCT | 1.05 | FG.Endth.FOS.FOSB | VWA1 | 1.73 | FG.Mono/cDC2.FCER1A.CD1C | MS4A7 | 1.02 |
| FG.EC.APOC3.APOA1 | SLC13A2 | 1.05 | FG.Endth.FOS.FOSB | THBD | 1.67 | FG.Mono/cDC2.FCER1A.CD1C | CSTA | 1.01 |
| FG.EC.APOC3.APOA1 | HSPB1 | 1.05 | FG.Endth.FOS.FOSB | PODXL | 1.67 | FG.Mono/cDC2.FCER1A.CD1C | CFP | 1.01 |
| FG.EC.APOC3.APOA1 | SLC6A19 | 1.04 | FG.Endth.FOS.FOSB | CD34 | 1.63 | FG.Mono/cDC2.FCER1A.CD1C | CD86 | 1.01 |
| FG.EC.APOC3.APOA1 | COMTD1 | 1.04 | FG.Endth.FOS.FOSB | JAM2 | 1.60 | FG.Mono/cDC2.FCER1A.CD1C | CLEC7A | 0.91 |
| FG.EC.APOC3.APOA1 | H2AFJ | 1.04 | FG.Endth.FOS.FOSB | CYYR1 | 1.58 | FG.Mono/cDC2.FCER1A.CD1C | P2RY13 | 0.88 |
| FG.EC.APOC3.APOA1 | MT2A | 1.04 | FG.Endth.FOS.FOSB | LDB2 | 1.54 | FG.Mono/cDC2.FCER1A.CD1C | MRC1 | 0.82 |
| FG.EC.APOC3.APOA1 | TREH | 1.04 | FG.Endth.FOS.FOSB | NOTCH4 | 1.50 | FG.Mono/cDC2.FCER1A.CD1C | HCK | 0.82 |
| FG.EC.APOC3.APOA1 | LGALS4 | 1.04 | FG.Endth.FOS.FOSB | KANK3 | 1.48 | FG.Mono/cDC2.FCER1A.CD1C | CD207 | 0.82 |
| FG.EC.APOC3.APOA1 | CLDN3 | 1.04 | FG.Endth.FOS.FOSB | CA4 | 1.41 | FG.Mono/cDC2.FCER1A.CD1C | CACNA2D3 | 0.71 |
| FG.EC.APOC3.APOA1 | ZG16 | 1.03 | FG.Endth.FOS.FOSB | TM4SF18 | 1.34 | FG.Mono/cDC2.FCER1A.CD1C | PILRA | 0.68 |
| FG.EC.APOC3.APOA1 | ARL14 | 1.02 | FG.Endth.FOS.FOSB | CDH5 | 1.31 | FG.Mono/cDC2.FCER1A.CD1C | FAM110A | 1.19 |
| FG.EC.APOC3.APOA1 | TSPAN8 | 1.02 | FG.Endth.FOS.FOSB | VWF | 1.31 | FG.Mono/cDC2.FCN1.S100A9 | S100A9 | 2.86 |
| FG.EC.APOC3.APOA1 | ACADVL | 1.01 | FG.Endth.FOS.FOSB | TMEM255B | 1.30 | FG.Mono/cDC2.FCN1.S100A9 | IL1B | 2.78 |
| FG.EC.APOC3.APOA1 | EFNA1 | 1.00 | FG.Endth.FOS.FOSB | ADGRL4 | 1.23 | FG.Mono/cDC2.FCN1.S100A9 | FCN1 | 2.78 |
| FG.EC.APOC3.APOA1 | COX5B | 0.99 | FG.Endth.FOS.FOSB | PTRF | 1.73 | FG.Mono/cDC2.FCN1.S100A9 | S100A8 | 2.23 |
| FG.EC.APOC3.APOA1 | DHDH | 0.98 | FG.Endth.FOS.FOSB | ECSCR-1 | 1.41 | FG.Mono/cDC2.FCN1.S100A9 | MNDA | 1.81 |
| FG.EC.APOC3.APOA1 | CTSD | 0.98 | FG.Endth.FOS.FOSB | NOV | 1.21 | FG.Mono/cDC2.FCN1.S100A9 | CFP | 1.57 |
| FG.EC.APOC3.APOA1 | AGPAT2 | 0.98 | FG.Endth/Art.FRZB.NOTCH3 | RGS5 | 4.08 | FG.Mono/cDC2.FCN1.S100A9 | CSTA | 1.56 |
| FG.EC.APOC3.APOA1 | SLC39A5 | 0.97 | FG.Endth/Art.FRZB.NOTCH3 | ACTA2 | 3.29 | FG.Mono/cDC2.FCN1.S100A9 | IL1RN | 1.43 |
| FG.EC.APOC3.APOA1 | LINC01133 | 0.97 | FG.Endth/Art.FRZB.NOTCH3 | SPARCL1 | 2.97 | FG.Mono/cDC2.FCN1.S100A9 | LINC01272 | 1.04 |
| FG.EC.APOC3.APOA1 | ACSL5 | 0.97 | FG.Endth/Art.FRZB.NOTCH3 | PLVAP | 2.81 | FG.Mono/cDC2.FCN1.S100A9 | OLR1 | 1.03 |
| FG.EC.APOC3.APOA1 | MISP | 0.95 | FG.Endth/Art.FRZB.NOTCH3 | IGFBP7 | 2.79 | FG.Mono/cDC2.FCN1.S100A9 | CD300E | 0.97 |
| FG.EC.APOC3.APOA1 | ESPN | 0.95 | FG.Endth/Art.FRZB.NOTCH3 | MYL9 | 2.77 | FG.Mono/cDC2.FCN1.S100A9 | LILRB2 | 0.92 |
| FG.EC.APOC3.APOA1 | C8G | 0.95 | FG.Endth/Art.FRZB.NOTCH3 | CALD1 | 2.53 | FG.Mono/cDC2.FCN1.S100A9 | FPR1 | 0.77 |
| FG.EC.APOC3.APOA1 | MTTP | 0.94 | FG.Endth/Art.FRZB.NOTCH3 | TAGLN | 2.52 | FG.Mono/Mac/cDC2.LYZ.CLEC10A | CPVL | 2.42 |
| FG.EC.APOC3.APOA1 | SLC22A18 | 0.94 | FG.Endth/Art.FRZB.NOTCH3 | CD36 | 2.50 | FG.Mono/Mac/cDC2.LYZ.CLEC10A | CLEC10A | 2.26 |
| FG.EC.APOC3.APOA1 | EPS8L2 | 0.94 | FG.Endth/Art.FRZB.NOTCH3 | CAV1 | 2.49 | FG.Mono/Mac/cDC2.LYZ.CLEC10A | IL1B | 2.22 |
| FG.EC.APOC3.APOA1 | TTC36 | 0.94 | FG.Endth/Art.FRZB.NOTCH3 | NDUFA4L2 | 2.34 | FG.Mono/Mac/cDC2.LYZ.CLEC10A | FCER1A | 2.05 |
| FG.EC.APOC3.APOA1 | EPS8L3 | 0.94 | FG.Endth/Art.FRZB.NOTCH3 | IFITM3 | 2.12 | FG.Mono/Mac/cDC2.LYZ.CLEC10A | C1QC | 2.03 |
| FG.EC.APOC3.APOA1 | MPST | 0.94 | FG.Endth/Art.FRZB.NOTCH3 | TPM2 | 2.12 | FG.Mono/Mac/cDC2.LYZ.CLEC10A | MNDA | 1.94 |
| FG.EC.APOC3.APOA1 | SLC39A4 | 0.93 | FG.Endth/Art.FRZB.NOTCH3 | SOD3 | 2.11 | FG.Mono/Mac/cDC2.LYZ.CLEC10A | MS4A6A | 1.94 |
| FG.EC.APOC3.APOA1 | MXD1 | 0.93 | FG.Endth/Art.FRZB.NOTCH3 | CRIP2 | 2.10 | FG.Mono/Mac/cDC2.LYZ.CLEC10A | IGSF6 | 1.64 |
| FG.EC.APOC3.APOA1 | FOLH1 | 0.93 | FG.Endth/Art.FRZB.NOTCH3 | FRZB | 2.08 | FG.Mono/Mac/cDC2.LYZ.CLEC10A | VSIG4 | 1.62 |
| FG.EC.APOC3.APOA1 | CLDN15 | 0.93 | FG.Endth/Art.FRZB.NOTCH3 | IFI27 | 2.08 | FG.Mono/Mac/cDC2.LYZ.CLEC10A | CD1E | 1.55 |
| FG.EC.APOC3.APOA1 | DHRS11 | 0.92 | FG.Endth/Art.FRZB.NOTCH3 | HIGD1B | 2.07 | FG.Mono/Mac/cDC2.LYZ.CLEC10A | CLEC7A | 1.15 |
| FG.EC.APOC3.APOA1 | CISD3 | 0.92 | FG.Endth/Art.FRZB.NOTCH3 | TSC22D1 | 2.00 | FG.Mono/Mac/cDC2.LYZ.CLEC10A | AOAH | 1.12 |
| FG.EC.APOC3.APOA1 | MIR194-2HG | 0.91 | FG.Endth/Art.FRZB.NOTCH3 | GNG11 | 2.00 | FG.Mono/Mac/cDC2.LYZ.CLEC10A | CLEC4A | 0.95 |
| FG.EC.APOC3.APOA1 | AKR1B10 | 0.90 | FG.Endth/Art.FRZB.NOTCH3 | AZM | 1.99 | FG.Mono/Mac/cDC2.LYZ.CLEC10A | P2RY13 | 1.00 |
| FG.EC.APOC3.APOA1 | HOOK2 | 0.90 | FG.Endth/Art.FRZB.NOTCH3 | TM4SF1 | 1.90 | FG.Mono/Mac/cDC2.LYZ.CLEC10A | CD86 | 1.20 |
| FG.EC.APOC3.APOA1 | PTGR1 | 0.90 | FG.Endth/Art.FRZB.NOTCH3 | MGP | 1.88 | FG.Mono/Mac/cDC2.LYZ.CLEC10A | PLAUR | 2.09 |
| FG.EC.APOC3.APOA1 | ETFB | 0.89 | FG.Endth/Art.FRZB.NOTCH3 | CLDN5 | 1.87 | FG.Mstcl.HPGDS.TPSAB1 | TPSB2 | 5.52 |
| FG.EC.APOC3.APOA1 | ID1 | 0.87 | FG.Endth/Art.FRZB.NOTCH3 | TIMP3 | 1.86 | FG.Mstcl.HPGDS.TPSAB1 | TPSAB1 | 5.18 |
| FG.EC.APOC3.APOA1 | MALL | 0.87 | FG.Endth/Art.FRZB.NOTCH3 | ARHGAP29 | 1.85 | FG.Mstcl.HPGDS.TPSAB1 | CTSG | 3.54 |
| FG.EC.APOC3.APOA1 | CLTB | 0.87 | FG.Endth/Art.FRZB.NOTCH3 | SLC9A3R2 | 1.85 | FG.Mstcl.HPGDS.TPSAB1 | HPGDS | 3.03 |
| FG.EC.APOC3.APOA1 | ATOX1 | 0.87 | FG.Endth/Art.FRZB.NOTCH3 | TINAGL1 | 1.79 | FG.Mstcl.HPGDS.TPSAB1 | CPA3 | 2.89 |
| FG.EC.APOC3.APOA1 | ALKBH7 | 0.86 | FG.Endth/Art.FRZB.NOTCH3 | COX4I2 | 1.75 | FG.Mstcl.HPGDS.TPSAB1 | ANXA1 | 2.85 |
| FG.EC.APOC3.APOA1 | RHOC | 0.86 | FG.Endth/Art.FRZB.NOTCH3 | RAMP2 | 1.75 | FG.Mstcl.HPGDS.TPSAB1 | MS4A2 | 2.57 |
| FG.EC.APOC3.APOA1 | ABHD11-AS1 | 0.86 | FG.Endth/Art.FRZB.NOTCH3 | CPE | 1.74 | FG.Mstcl.HPGDS.TPSAB1 | VWA5A | 2.44 |
| FG.EC.APOC3.APOA1 | FAM132A | 0.86 | FG.Endth/Art.FRZB.NOTCH3 | SPARC | 1.74 | FG.Mstcl.HPGDS.TPSAB1 | HDC | 2.21 |
| FG.EC.APOC3.APOA1 | CIDEB | 0.85 | FG.Endth/Art.FRZB.NOTCH3 | EPAS1 | 1.72 | FG.Mstcl.HPGDS.TPSAB1 | NFKBIA | 2.16 |
| FG.EC.APOC3.APOA1 | TMEM220 | 0.84 | FG.Endth/Art.FRZB.NOTCH3 | PRSS23 | 1.71 | FG.Mstcl.HPGDS.TPSAB1 | SLC18A2 | 1.98 |
| FG.EC.APOC3.APOA1 | NDUFB7 | 0.84 | FG.Endth/Art.FRZB.NOTCH3 | TMEM88 | 1.70 | FG.Mstcl.HPGDS.TPSAB1 | KIT | 1.98 |
| FG.EC.APOC3.APOA1 | MYO15B | 0.84 | FG.Endth/Art.FRZB.NOTCH3 | CSRP2 | 1.69 | FG.Mstcl.HPGDS.TPSAB1 | CMA1 | 1.93 |
| FG.EC.APOC3.APOA1 | EPCAM | 0.84 | FG.Endth/Art.FRZB.NOTCH3 | FABP4 | 1.66 | FG.Mstcl.HPGDS.TPSAB1 | FCER1G | 1.74 |
| FG.EC.APOC3.APOA1 | S100A10 | 0.84 | FG.Endth/Art.FRZB.NOTCH3 | ESAM | 1.65 | FG.Mstcl.HPGDS.TPSAB1 | KRT1 | 1.72 |
| FG.EC.APOC3.APOA1 | COX4I1 | 0.83 | FG.Endth/Art.FRZB.NOTCH3 | GPX3 | 1.64 | FG.Mstcl.HPGDS.TPSAB1 | IL1RL1 | 1.67 |
| FG.EC.APOC3.APOA1 | GUCA2B | 0.83 | FG.Endth/Art.FRZB.NOTCH3 | PTRF | 1.64 | FG.Mstcl.HPGDS.TPSAB1 | FTH1 | 1.66 |
| FG.EC.APOC3.APOA1 | ATP5I | 0.83 | FG.Endth/Art.FRZB.NOTCH3 | STEAP4 | 1.63 | FG.Mstcl.HPGDS.TPSAB1 | GATA2 | 1.63 |
| FG.EC.APOC3.APOA1 | MYH14 | 0.82 | FG.Endth/Art.FRZB.NOTCH3 | MFGE8 | 1.59 | FG.Mstcl.HPGDS.TPSAB1 | RP11-354E11.2 | 1.33 |
| FG.EC.APOC3.APOA1 | HYI | 0.82 | FG.Endth/Art.FRZB.NOTCH3 | STOM | 1.59 | FG.Mstcl.HPGDS.TPSAB1 | AC004791.2 | 1.31 |
| FG.EC.APOC3.APOA1 | NAPRT | 0.82 | FG.Endth/Art.FRZB.NOTCH3 | PRKCDBP | 1.56 | FG.Mstcl.HPGDS.TPSAB1 | H3F3B | 1.48 |
| FG.EC.APOC3.APOA1 | OAT | 0.79 | FG.Endth/Art.FRZB.NOTCH3 | NOTCH3 | 1.56 | FG.MyoFibro.DES.MYH11 | TAGLN | 4.22 |
| FG.EC.APOC3.APOA1 | LGALS3 | 0.78 | FG.Endth/Art.FRZB.NOTCH3 | FLT1 | 1.50 | FG.MyoFibro.DES.MYH11 | ACTA2 | 4.16 |
| FG.EC.APOC3.APOA1 | NDUFS7 | 0.78 | FG.Endth/Art.FRZB.NOTCH3 | SDPR | 1.49 | FG.MyoFibro.DES.MYH11 | ACTG2 | 3.79 |
| FG.EC.APOC3.APOA1 | COX6B1 | 0.77 | FG.Endth/Art.FRZB.NOTCH3 | PDGFRB | 1.49 | FG.MyoFibro.DES.MYH11 | TPM2 | 3.43 |
| FG.EC.APOC3.APOA1 | TM6SF2 | 0.77 | FG.Endth/Art.FRZB.NOTCH3 | COX7A1 | 1.48 | FG.MyoFibro.DES.MYH11 | MYL9 | 3.36 |
| FG.EC.APOC3.APOA1 | RP11-467L13.7 | 0.77 | FG.Endth/Art.FRZB.NOTCH3 | EMCN | 1.46 | FG.MyoFibro.DES.MYH11 | SOSTDC1 | 2.99 |
| FG.EC.APOC3.APOA1 | BLVRB | 0.76 | FG.Endth/Art.FRZB.NOTCH3 | NR2F2 | 1.42 | FG.MyoFibro.DES.MYH11 | MYLK | 2.96 |
| FG.EC.APOC3.APOA1 | ARL6IP4 | 0.76 | FG.Endth/Art.FRZB.NOTCH3 | RBP7 | 1.41 | FG.MyoFibro.DES.MYH11 | MYH11 | 2.94 |
| FG.EC.APOC3.APOA1 | SPINT1 | 0.76 | FG.Endth/Art.FRZB.NOTCH3 | CAV2 | 1.39 | FG.MyoFibro.DES.MYH11 | IGFBP5 | 2.85 |
| FG.EC.APOC3.APOA1 | MS4A8 | 0.76 | FG.Endth/Art.FRZB.NOTCH3 | BCAM | 1.38 | FG.MyoFibro.DES.MYH11 | HHIP | 2.54 |
| FG.EC.APOC3.APOA1 | TPM1 | 0.75 | FG.Endth/Art.FRZB.NOTCH3 | EGFL7 | 1.33 | FG.MyoFibro.DES.MYH11 | CNN1 | 2.25 |
| FG.EC.APOC3.APOA1 | SLC25A1 | 0.75 | FG.Endth/Art.FRZB.NOTCH3 | RAMP3 | 1.31 | FG.MyoFibro.DES.MYH11 | DES | 1.89 |
| FG.EC.APOC3.APOA1 | P4HB | 0.74 | FG.Endth/Art.FRZB.NOTCH3 | PPP1R14A | 1.30 | FG.MyoFibro.DES.MYH11 | LMOD1 | 1.84 |
| FG.EC.APOC3.APOA1 | TMC5 | 0.74 | FG.Endth/Art.FRZB.NOTCH3 | LHFP | 1.29 | FG.MyoFibro.DES.MYH11 | CARMN | 1.54 |
| FG.EC.APOC3.APOA1 | FAM3B | 0.74 | FG.Endth/Art.FRZB.NOTCH3 | ADGRF5 | 1.27 | FG.MyoFibro.DES.MYH11 | PLN | 1.35 |
| FG.EC.APOC3.APOA1 | ERICH4 | 0.74 | FG.Endth/Art.FRZB.NOTCH3 | COL4A1 | 1.27 | FG.MyoFibro.DES.MYH11 | HSD17B6 | 1.16 |
| FG.EC.APOC3.APOA1 | PLS1 | 0.73 | FG.Endth/Art.FRZB.NOTCH3 | COL4A2 | 1.27 | FG.Paneth.ITLN2.PRSS2 | DEFA6 | 6.60 |
| FG.EC.APOC3.APOA1 | SLC25A37 | 0.73 | FG.Endth/Art.FRZB.NOTCH3 | BGN | 1.24 | FG.Paneth.ITLN2.PRSS2 | REG3A | 6.57 |
| FG.EC.APOC3.APOA1 | FIS1 | 0.73 | FG.Endth/Art.FRZB.NOTCH3 | VWA1 | 1.22 | FG.Paneth.ITLN2.PRSS2 | PRSS2 | 5.67 |
| FG.EC.APOC3.APOA1 | CA2 | 0.73 | FG.Endth/Art.FRZB.NOTCH3 | TM4SF18 | 1.16 | FG.Paneth.ITLN2.PRSS2 | PLA2G2A | 5.34 |
| FG.EC.APOC3.APOA1 | GPA33 | 0.72 | FG.Endth/Art.FRZB.NOTCH3 | LIFR | 1.15 | FG.Paneth.ITLN2.PRSS2 | ITLN2 | 4.21 |
| FG.EC.APOC3.APOA1 | CHP2 | 0.72 | FG.Endth/Art.FRZB.NOTCH3 | HSPG2 | 1.15 | FG.Paneth.ITLN2.PRSS2 | LYZ | 3.92 |
| FG.EC.APOC3.APOA1 | CMBL | 0.72 | FG.Endth/Art.FRZB.NOTCH3 | SORBS2 | 1.14 | FG.Paneth.ITLN2.PRSS2 | TFF3 | 2.89 |
| FG.EC.APOC3.APOA1 | MTRNR2L1 | 0.72 | FG.Endth/Art.FRZB.NOTCH3 | MYO1B | 1.14 | FG.Paneth.ITLN2.PRSS2 | GUCAZA | 2.72 |
| FG.EC.APOC3.APOA1 | OTOP3 | 0.72 | FG.Endth/Art.FRZB.NOTCH3 | CYYR1 | 1.13 | FG.Paneth.ITLN2.PRSS2 | SPINK1 | 1.89 |
| FG.EC.APOC3.APOA1 | KRT20 | 0.71 | FG.Endth/Art.FRZB.NOTCH3 | RCAN2 | 1.11 | FG.Paneth.ITLN2.PRSS2 | SERPINA1 | 1.89 |
| FG.EC.APOC3.APOA1 | AQP10 | 0.71 | FG.Endth/Art.FRZB.NOTCH3 | DLC1 | 1.09 | FG.Paneth.ITLN2.PRSS2 | RP11-1143G9.4 | 1.81 |
| FG.EC.APOC3.APOA1 | GSTA1 | 0.71 | FG.Endth/Art.FRZB.NOTCH3 | FAM13C | 1.07 | FG.Paneth.ITLN2.PRSS2 | IGFBP2 | 1.15 |
| FG.EC.APOC3.APOA1 | HSD17B2 | 0.71 | FG.Endth/Art.FRZB.NOTCH3 | NOV | 1.04 | FG.Paneth.ITLN2.PRSS2 | AZGP1 | 1.06 |
| FG.EC.APOC3.APOA1 | VIL1 | 0.71 | FG.Endth/Art.FRZB.NOTCH3 | PODXL | 1.03 | FG.Paneth.ITLN2.PRSS2 | KLK12 | 0.86 |
| FG.EC.APOC3.APOA1 | EFHD2 | 0.71 | FG.Endth/Art.FRZB.NOTCH3 | JAM2 | 1.00 | FG.Paneth.ITLN2.PRSS2 | MUC1 | 0.62 |
| FG.EC.APOC3.APOA1 | CHCHD10 | 0.71 | FG.Endth/Art.FRZB.NOTCH3 | ADGRL4 | 0.99 | FG.Paneth.ITLN2.PRSS2 | S100A1 | 0.58 |
| FG.EC.APOC3.APOA1 | ACE2 | 0.70 | FG.Endth/Art.FRZB.NOTCH3 | TMEM47 | 0.96 | FG.Paneth.ITLN2.PRSS2 | SMOC2 | 0.56 |
| FG.EC.APOC3.APOA1 | MYO7B | 0.70 | FG.Endth/Art.FRZB.NOTCH3 | MMRN2 | 0.94 | FG.Paneth.ITLN2.PRSS2 | PCSK1 | 0.51 |
| FG.EC.APOC3.APOA1 | CIDEC | 0.70 | FG.Endth/Art.FRZB.NOTCH3 | FAM162B | 0.91 | FG.Paneth.ITLN2.PRSS2 | HEPACAM2 | 0.48 |
| FG.EC.APOC3.APOA1 | NDUFS6 | 0.70 | FG.Endth/Art.FRZB.NOTCH3 | SNCG | 0.90 | FG.Paneth.ITLN2.PRSS2 | LRRC26 | 0.41 |
| FG.EC.APOC3.APOA1 | MT1E | 0.69 | FG.Endth/Art.FRZB.NOTCH3 | THBD | 0.90 | FG.Paneth.ITLN2.PRSS2 | KLK11 | 0.39 |
| FG.EC.APOC3.APOA1 | CDH17 | 0.69 | FG.Endth/Art.FRZB.NOTCH3 | CD34 | 0.90 | FG.Paneth.ITLN2.PRSS2 | SLCO4A1-AS1 | 0.30 |
| FG.EC.APOC3.APOA1 | UGT2B17 | 0.69 | FG.Endth/Art.FRZB.NOTCH3 | MCAM | 0.88 | FG.Paneth.ITLN2.PRSS2 | ATOH1 | 0.29 |
| FG.EC.APOC3.APOA1 | TMEM54 | 0.69 | FG.Endth/Art.FRZB.NOTCH3 | PTPRB | 0.87 | FG.Paneth.ITLN2.PRSS2 | LINC00261 | 0.41 |
| FG.EC.APOC3.APOA1 | ANKRD9 | 0.69 | FG.Endth/Art.FRZB.NOTCH3 | SLC14A1 | 0.87 | FG.Plsma/IgA.IGHA1.AL9287683 | AL928768.3 | 1.58 |
| FG.EC.APOC3.APOA1 | SFN | 0.69 | FG.Endth/Art.FRZB.NOTCH3 | LDB2 | 0.84 | FG.Plsma/IgA.IGHA1.AL9287683 | MZB1 | 1.55 |
| FG.EC.APOC3.APOA1 | SERINC2 | 0.69 | FG.Endth/Art.FRZB.NOTCH3 | KANK3 | 0.83 | FG.Plsma/IgA.IGHA1.AL9287683 | TNFRSF17 | 1.30 |
| FG.EC.APOC3.APOA1 | ATF3 | 0.68 | FG.Endth/Art.FRZB.NOTCH3 | VWF | 0.79 | FG.Plsma/IgA.IGHA1.AL9287683 | DERL3 | 1.22 |
| FG.EC.APOC3.APOA1 | HEBP1 | 0.68 | FG.Endth/Art.FRZB.NOTCH3 | GJA4 | 0.77 | FG.Plsma/IgA.IGHA1.AL9287683 | FKBP11 | 1.09 |
| FG.EC.APOC3.APOA1 | RBBP8NL | 0.68 | FG.Endth/Art.FRZB.NOTCH3 | EDNRA | 0.77 | FG.Plsma/IgA.IGHA1.AL9287683 | CCR10 | 0.48 |
| FG.EC.APOC3.APOA1 | DSP | 0.68 | FG.Endth/Art.FRZB.NOTCH3 | ECSCR-1 | 0.75 | FG.Plsma/IgA.IGHA2.APOA1 | IGHA2 | 4.06 |
| FG.EC.APOC3.APOA1 | GATM | 0.68 | FG.Endth/Art.FRZB.NOTCH3 | SLCO2A1 | 0.70 | FG.Plsma/IgA.IGHA2.APOA1 | IGKC | 2.71 |
| FG.EC.APOC3.APOA1 | SULT1B1 | 0.67 | FG.Endth/Art.FRZB.NOTCH3 | KDR | 0.67 | FG.Plsma/IgA.IGHA2.APOA1 | IGHA1 | 2.22 |
| FG.EC.APOC3.APOA1 | BAIAP2L2 | 0.67 | FG.Endth/Art.FRZB.NOTCH3 | FAM107A | 0.55 | FG.Plsma/IgA.IGHA2.APOA1 | JCHAIN | 1.90 |
| FG.EC.APOC3.APOA1 | AAMDC | 0.67 | FG.Endth/Art.FRZB.NOTCH3 | FERMT2 | 0.79 | FG.Plsma/IgA.IGHA2.APOA1 | MZB1 | 1.54 |
| FG.EC.APOC3.APOA1 | CTSE | 0.66 | FG.Endth/Art.FRZB.NOTCH3 | ITGA1 | 1.22 | FG.Plsma/IgA.IGHA2.APOA1 | SSR4 | 1.35 |
| FG.EC.APOC3.APOA1 | PRSS8 | 0.66 | FG.Endth/Art.FRZB.NOTCH3 | CD320 | 2.52 | FG.Plsma/IgA.IGHA2.APOA1 | DERL3 | 1.31 |
| FG.EC.APOC3.APOA1 | AC004754.3 | 0.65 | FG.Endth/Art.FRZB.NOTCH3 | NKX2-3 | 1.02 | FG.Plsma/IgA.IGHA2.APOA1 | TNFRSF17 | 1.26 |
| FG.EC.APOC3.APOA1 | USH1C | 0.65 | FG.Endth/Art.FRZB.NOTCH3 | CALCRL | 0.93 | FG.Plsma/IgA.IGHA2.APOA1 | FKBP11 | 1.14 |
| FG.EC.APOC3.APOA1 | DST | 0.65 | FG.Endth/Art.FRZB.NOTCH3 | GSN | 1.76 | FG.Plsma/IgA.IGHA2.APOA1 | XBP1 | 0.99 |
| FG.EC.APOC3.APOA1 | GLTPD2 | 0.65 | FG.Endth/Art.FRZB.NOTCH3 | SYNPO2 | 1.11 | FG.Plsma/IgA.IGHA2.APOA1 | AL928768.3 | 0.91 |
| FG.EC.APOC3.APOA1 | DNPH1 | 0.64 | FG.Endth/Art.FRZB.NOTCH3 | GPRC5B | 0.61 | FG.Plsma/IgA.IGHA2.APOA1 | IGKV1-12 | 0.89 |
| FG.EC.APOC3.APOA1 | COX7B | 0.64 | FG.Endth/Cap.PLVAP.FLT1 | PLVAP | 3.73 | FG.Plsma/IgA.IGHA2.APOA1 | PRDX4 | 0.80 |
| FG.EC.APOC3.APOA1 | ANGPTL4 | 0.64 | FG.Endth/Cap.PLVAP.FLT1 | FABP5 | 3.48 | FG.Plsma/IgA.IGHA2.APOA1 | SEC11C | 0.74 |
| FG.EC.APOC3.APOA1 | ACADS | 0.64 | FG.Endth/Cap.PLVAP.FLT1 | SPARCL1 | 3.20 | FG.Plsma/IgA.IGHA2.APOA1 | SSR3 | 0.66 |
| FG.EC.APOC3.APOA1 | MEIS2 | 0.64 | FG.Endth/Cap.PLVAP.FLT1 | IFI27 | 3.04 | FG.Plsma/IgA.IGHA2.APOA1 | RRBP1 | 0.61 |
| FG.EC.APOC3.APOA1 | BLOC1S1 | 0.64 | FG.Endth/Cap.PLVAP.FLT1 | CD320 | 3.03 | FG.Plsma/IgA.IGHA2.APOA1 | SDC1 | 0.55 |
| FG.EC.APOC3.APOA1 | FUT2 | 0.64 | FG.Endth/Cap.PLVAP.FLT1 | RAMP2 | 2.90 | FG.Plsma/IgA.IGHA2.APOA1 | CCR10 | 0.42 |
| FG.EC.APOC3.APOA1 | CBLC | 0.63 | FG.Endth/Cap.PLVAP.FLT1 | IGFBP7 | 2.81 | FG.Plsma/IgA.IGHA2.APOA1 | AMPD1 | 0.40 |
| FG.EC.APOC3.APOA1 | FCGRT | 0.63 | FG.Endth/Cap.PLVAP.FLT1 | CAV1 | 2.81 | FG.Plsma/IgA.IGHA2.APOA1 | FCRL5 | 0.39 |
| FG.EC.APOC3.APOA1 | NR0B2 | 0.63 | FG.Endth/Cap.PLVAP.FLT1 | GNG11 | 2.81 | FG.Plsma/IgA.IGHA2.APOA1 | SLAMF7 | 0.38 |
| FG.EC.APOC3.APOA1 | GDA | 0.62 | FG.Endth/Cap.PLVAP.FLT1 | CD36 | 2.67 | FG.Plsma/IgA.IGHA2.APOA1 | PDK1 | 0.30 |
| FG.EC.APOC3.APOA1 | ABCG5 | 0.62 | FG.Endth/Cap.PLVAP.FLT1 | GSN | 2.62 | FG.Plsma/IgA.IGHA2.APOA1 | RP11-290F5.1 | 0.29 |
| FG.EC.APOC3.APOA1 | SDCBP2 | 0.62 | FG.Endth/Cap.PLVAP.FLT1 | CLDN5 | 2.55 | FG.Plsma/IgA.IGHA2.APOA1 | RP11-492E3.2 | 0.27 |
| FG.EC.APOC3.APOA1 | FAM46A | 0.62 | FG.Endth/Cap.PLVAP.FLT1 | SLC9A3R2 | 2.55 | FG.Plsma/IgA.IGKC.AL9287683 | IGKC | 2.89 |
| FG.EC.APOC3.APOA1 | ATP1A1 | 0.62 | FG.Endth/Cap.PLVAP.FLT1 | CRIP2 | 2.54 | FG.Plsma/IgA.IGKC.AL9287683 | IGHA1 | 2.65 |
| FG.EC.APOC3.APOA1 | RNF186 | 0.62 | FG.Endth/Cap.PLVAP.FLT1 | TM4SF1 | 2.48 | FG.Plsma/IgA.IGKC.AL9287683 | JCHAIN | 2.58 |
| FG.EC.APOC3.APOA1 | TNFRSF12A | 0.61 | FG.Endth/Cap.PLVAP.FLT1 | IFITM3 | 2.46 | FG.Plsma/IgA.IGKC.AL9287683 | AL928768.3 | 1.60 |
| FG.EC.APOC3.APOA1 | VEGFA | 0.61 | FG.Endth/Cap.PLVAP.FLT1 | IGFBP4 | 2.39 | FG.Plsma/IgA.IGKC.AL9287683 | MZB1 | 1.56 |
| FG.EC.APOC3.APOA1 | ASS1 | 0.61 | FG.Endth/Cap.PLVAP.FLT1 | MGP | 2.35 | FG.Plsma/IgA.IGKC.AL9287683 | JUN | 1.32 |
| FG.EC.APOC3.APOA1 | ARHGEF16 | 0.59 | FG.Endth/Cap.PLVAP.FLT1 | RNASE1 | 2.23 | FG.Plsma/IgA.IGKC.AL9287683 | TNFRSF17 | 1.25 |
| FG.EC.APOC3.APOA1 | PPP1R14D | 0.59 | FG.Endth/Cap.PLVAP.FLT1 | TMEM88 | 2.23 | FG.Plsma/IgA.IGKC.AL9287683 | SSR4 | 1.22 |
| FG.EC.APOC3.APOA1 | TMEM25 | 0.59 | FG.Endth/Cap.PLVAP.FLT1 | FLT1 | 2.23 | FG.Plsma/IgA.IGKC.AL9287683 | DERL3 | 1.21 |
| FG.EC.APOC3.APOA1 | PEBP1 | 0.58 | FG.Endth/Cap.PLVAP.FLT1 | A2M | 2.20 | FG.Plsma/IgA.IGKC.AL9287683 | HSP90B1 | 1.15 |
| FG.EC.APOC3.APOA1 | RAB17 | 0.58 | FG.Endth/Cap.PLVAP.FLT1 | PLAT | 2.17 | FG.Plsma/IgA.IGKC.AL9287683 | XBP1 | 1.11 |
| FG.EC.APOC3.APOA1 | XPNPEP2 | 0.58 | FG.Endth/Cap.PLVAP.FLT1 | ENPP2 | 2.15 | FG.Plsma/IgA.IGKC.AL9287683 | FKBP11 | 1.10 |
| FG.EC.APOC3.APOA1 | PRR13 | 0.58 | FG.Endth/Cap.PLVAP.FLT1 | EMCN | 2.14 | FG.Plsma/IgA.IGKC.AL9287683 | HSPA1A | 1.02 |
| FG.EC.APOC3.APOA1 | TMEM82 | 0.57 | FG.Endth/Cap.PLVAP.FLT1 | PRSS23 | 2.06 | FG.Plsma/IgA.IGKC.AL9287683 | SEC11C | 0.98 |
| FG.EC.APOC3.APOA1 | TCEB2 | 0.56 | FG.Endth/Cap.PLVAP.FLT1 | VAMP5 | 2.06 | FG.Plsma/IgA.IGKC.AL9287683 | HERPUD1 | 0.97 |
| FG.EC.APOC3.APOA1 | LAMB3 | 0.56 | FG.Endth/Cap.PLVAP.FLT1 | CA4 | 2.04 | FG.Plsma/IgA.IGKC.AL9287683 | SPCS2 | 0.90 |
| FG.EC.APOC3.APOA1 | OSGIN1 | 0.55 | FG.Endth/Cap.PLVAP.FLT1 | CD59 | 2.01 | FG.Plsma/IgA.IGKC.AL9287683 | DNAJB9 | 0.86 |
| FG.EC.APOC3.APOA1 | IFI27 | 0.55 | FG.Endth/Cap.PLVAP.FLT1 | ESAM | 2.00 | FG.Plsma/IgA.IGKC.AL9287683 | PRDX4 | 0.80 |
| FG.EC.APOC3.APOA1 | NPNT | 0.55 | FG.Endth/Cap.PLVAP.FLT1 | PECAM1 | 2.00 | FG.Plsma/IgA.IGKC.AL9287683 | VIMP | 0.78 |
| FG.EC.APOC3.APOA1 | LGALS3BP | 0.54 | FG.Endth/Cap.PLVAP.FLT1 | HSPG2 | 2.00 | FG.Plsma/IgA.IGKC.AL9287683 | PDIA4 | 0.76 |
| FG.EC.APOC3.APOA1 | MEP1B | 0.53 | FG.Endth/Cap.PLVAP.FLT1 | SDPR | 2.00 | FG.Plsma/IgA.IGKC.AL9287683 | HSPA5 | 0.75 |
| FG.EC.APOC3.APOA1 | CCL25 | 0.52 | FG.Endth/Cap.PLVAP.FLT1 | EGFL7 | 2.00 | FG.Plsma/IgA.IGKC.AL9287683 | UBE2J1 | 0.70 |
| FG.EC.APOC3.APOA1 | FBXO2 | 0.51 | FG.Endth/Cap.PLVAP.FLT1 | RAMP3 | 1.98 | FG.Plsma/IgA.IGKC.AL9287683 | FKBP2 | 0.64 |
| FG.EC.APOC3.APOA1 | UGT1A1 | 0.49 | FG.Endth/Cap.PLVAP.FLT1 | BCAM | 1.94 | FG.Plsma/IgA.IGKC.AL9287683 | ITM2C | 0.61 |
| FG.EC.APOC3.APOA1 | ENTPD8 | 0.45 | FG.Endth/Cap.PLVAP.FLT1 | IGFBP3 | 1.94 | FG.Plsma/IgA.IGKC.AL9287683 | PDIA6 | 0.61 |
| FG.EC.APOC3.APOA1 | FTL | 0.34 | FG.Endth/Cap.PLVAP.FLT1 | ARHGAP29 | 1.92 | FG.Plsma/IgA.IGKC.AL9287683 | DUSP5 | 0.60 |
| FG.EC.APOC3.APOA1 | SEPP1 | 0.25 | FG.Endth/Cap.PLVAP.FLT1 | PTRF | 1.89 | FG.Plsma/IgA.IGKC.AL9287683 | SSR3 | 0.58 |
| FG.EC.APOC3.APOA1 | AURKAIP1 | 0.64 | FG.Endth/Cap.PLVAP.FLT1 | ENG | 1.87 | FG.Plsma/IgA.IGKC.AL9287683 | SDF2L1 | 0.57 |
| FG.EC.APOC3.APOA1 | RIOK3 | 0.64 | FG.Endth/Cap.PLVAP.FLT1 | TGFBR2 | 1.86 | FG.Plsma/IgA.IGKC.AL9287683 | CRELD2 | 0.55 |
| FG.EC.APOC3.APOA1 | ABCG2 | 0.54 | FG.Endth/Cap.PLVAP.FLT1 | CLEC14A | 1.85 | FG.Plsma/IgA.IGKC.AL9287683 | SDC1 | 0.54 |
| FG.EC.APOC3.APOA1 | CYP4F12 | 0.56 | FG.Endth/Cap.PLVAP.FLT1 | NFIB | 1.84 | FG.Plsma/IgA.IGKC.AL9287683 | SPCS3 | 0.52 |
| FG.EC.APOC3.APOA1 | MST1 | 0.54 | FG.Endth/Cap.PLVAP.FLT1 | TM4SF18 | 1.81 | FG.Plsma/IgA.IGKC.AL9287683 | TRAM1 | 0.52 |
| FG.EC.APOC3.APOA1 | PKP3 | 0.58 | FG.Endth/Cap.PLVAP.FLT1 | SPARC | 1.81 | FG.Plsma/IgA.IGKC.AL9287683 | MANF | 0.51 |
| FG.EC.APOC3.APOA1 | HLA-DRB5 | 0.27 | FG.Endth/Cap.PLVAP.FLT1 | JAM2 | 1.80 | FG.Plsma/IgA.IGKC.AL9287683 | UAP1 | 0.50 |
| FG.EC.APOC3.APOA1 | SLC35G1 | 0.54 | FG.Endth/Cap.PLVAP.FLT1 | ADGRL4 | 1.78 | FG.Plsma/IgA.IGKC.AL9287683 | CCR10 | 0.48 |
| FG.EC.APOC3.APOA1 | NDUFB10 | 0.61 | FG.Endth/Cap.PLVAP.FLT1 | RBP7 | 1.77 | FG.Plsma/IgA.IGKC.AL9287683 | FAM46C | 0.46 |
| FG.EC.APOC3.APOA1 | ADH4 | 0.55 | FG.Endth/Cap.PLVAP.FLT1 | PCAT19 | 1.77 | FG.Plsma/IgA.IGKC.AL9287683 | ANKRD28 | 0.45 |
| FG.EC.C15orf48.OLFM4 | REG1A | 3.32 | FG.Endth/Cap.PLVAP.FLT1 | VWA1 | 1.76 | FG.Plsma/IgA.IGKC.AL9287683 | CHPF | 0.45 |
| FG.EC.C15orf48.OLFM4 | MT1G | 2.76 | FG.Endth/Cap.PLVAP.FLT1 | EPAS1 | 1.75 | FG.Plsma/IgA.IGKC.AL9287683 | TXNDC11 | 0.44 |
| FG.EC.C15orf48.OLFM4 | OLFM4 | 2.74 | FG.Endth/Cap.PLVAP.FLT1 | CALCRL | 1.74 | FG.Plsma/IgA.IGKC.AL9287683 | DNAJC3 | 0.44 |
| FG.EC.C15orf48.OLFM4 | MT1E | 2.58 | FG.Endth/Cap.PLVAP.FLT1 | PODXL | 1.73 | FG.Plsma/IgA.IGKC.AL9287683 | IGHA2 | 0.43 |
| FG.EC.C15orf48.OLFM4 | SPINK1 | 2.51 | FG.Endth/Cap.PLVAP.FLT1 | LIFR | 1.72 | FG.Plsma/IgA.IGKC.AL9287683 | GNG7 | 0.41 |
| FG.EC.C15orf48.OLFM4 | C15orf48 | 2.41 | FG.Endth/Cap.PLVAP.FLT1 | VWF | 1.71 | FG.Plsma/IgA.IGKC.AL9287683 | PLPP5 | 0.40 |
| FG.EC.C15orf48.OLFM4 | KRT18 | 2.38 | FG.Endth/Cap.PLVAP.FLT1 | EHD4 | 1.71 | FG.Plsma/IgA.IGKC.AL9287683 | MEI1 | 0.40 |
| FG.EC.C15orf48.OLFM4 | MT1H | 2.30 | FG.Endth/Cap.PLVAP.FLT1 | STOM | 1.71 | FG.Plsma/IgA.IGKC.AL9287683 | CCPG1 | 0.40 |
| FG.EC.C15orf48.OLFM4 | KRT8 | 2.23 | FG.Endth/Cap.PLVAP.FLT1 | IGFBP6 | 1.67 | FG.Plsma/IgA.IGKC.AL9287683 | SLAMF7 | 0.36 |
| FG.EC.C15orf48.OLFM4 | AGR2 | 2.15 | FG.Endth/Cap.PLVAP.FLT1 | NPDC1 | 1.66 | FG.Plsma/IgA.IGKC.AL9287683 | AMPD1 | 0.32 |
| FG.EC.C15orf48.OLFM4 | TSPAN8 | 2.04 | FG.Endth/Cap.PLVAP.FLT1 | TIMP3 | 1.66 | FG.Plsma/IgA.IGKC.AL9287683 | NUCB2 | 0.35 |
| FG.EC.C15orf48.OLFM4 | KRT19 | 2.04 | FG.Endth/Cap.PLVAP.FLT1 | PLPP1 | 1.65 | FG.Plsma/IgA.IGKC.AL9287683 | WNT10A | 0.29 |
| FG.EC.C15orf48.OLFM4 | MT1F | 2.03 | FG.Endth/Cap.PLVAP.FLT1 | CTGF | 1.65 | FG.Plsma/IgA.IGKV1-12.IGKV3-20 | IGKV1-12 | 3.66 |
| FG.EC.C15orf48.OLFM4 | LGALS4 | 1.96 | FG.Endth/Cap.PLVAP.FLT1 | ECSCR-1 | 1.63 | FG.Plsma/IgA.IGKV3-20.IGKV4-1 | IGKV3-20 | 2.19 |
| FG.EC.C15orf48.OLFM4 | PHGR1 | 1.88 | FG.Endth/Cap.PLVAP.FLT1 | C8orf4 | 1.63 | FG.Plsma/IgA.IGKV3-20.IGKV4-1 | IGKV1-5 | 2.16 |
| FG.EC.C15orf48.OLFM4 | GPX2 | 1.74 | FG.Endth/Cap.PLVAP.FLT1 | CD34 | 1.59 | FG.Plsma/IgA.IGKV3-20.IGKV4-1 | IGKV4-1 | 2.11 |
| FG.EC.C15orf48.OLFM4 | LGALS2 | 1.73 | FG.Endth/Cap.PLVAP.FLT1 | CAV2 | 1.58 | FG.Plsma/IgA.IGKV3-20.IGKV4-1 | IGKV3-11 | 1.65 |
| FG.EC.C15orf48.OLFM4 | MT1M | 1.64 | FG.Endth/Cap.PLVAP.FLT1 | PTPRB | 1.56 | FG.Plsma/IgA.IGKV3-20.IGKV4-1 | MZB1 | 1.61 |
| FG.EC.C15orf48.OLFM4 | MGST1 | 1.53 | FG.Endth/Cap.PLVAP.FLT1 | NOTCH4 | 1.55 | FG.Plsma/IgA.IGKV3-20.IGKV4-1 | AL928768.3 | 1.52 |
| FG.EC.C15orf48.OLFM4 | AGR3 | 1.46 | FG.Endth/Cap.PLVAP.FLT1 | RBP5 | 1.52 | FG.Plsma/IgA.IGKV3-20.IGKV4-1 | IGHV3-23 | 0.82 |
| FG.EC.C15orf48.OLFM4 | TFF3 | 1.25 | FG.Endth/Cap.PLVAP.FLT1 | GIMAP4 | 1.48 | FG.Plsma/IgA.IGKV3-20.IGKV4-1 | IGKV1-39 | 0.73 |
| FG.EC.C15orf48.OLFM4 | PPP1R1B | 1.24 | FG.Endth/Cap.PLVAP.FLT1 | C10orf10 | 1.48 | FG.Plsma/IgA.IGKV3-20.IGKV4-1 | IGHV3-74 | 0.62 |
| FG.EC.C15orf48.OLFM4 | KCNJ13 | 1.12 | FG.Endth/Cap.PLVAP.FLT1 | SORBS2 | 1.47 | FG.Plsma/IgA.IGKV3-20.IGKV4-1 | IGHV5-51 | 0.51 |
| FG.EC.C15orf48.OLFM4 | PLA2G2A | 1.11 | FG.Endth/Cap.PLVAP.FLT1 | COL15A1 | 1.46 | FG.Plsma/IgA.IGKV3-20.IGKV4-1 | IGHV3-48 | 0.50 |
| FG.EC.C15orf48.OLFM4 | C2orf82 | 0.68 | FG.Endth/Cap.PLVAP.FLT1 | COX7A1 | 1.45 | FG.Plsma/IgA.IGKV3-20.IGKV4-1 | IGHV3-15 | 0.48 |
| FG.EC.C15orf48.OLFM4 | C10orf99 | 0.67 | FG.Endth/Cap.PLVAP.FLT1 | CYYR1 | 1.44 | FG.Plsma/IgA.IGKV3-20.IGKV4-1 | IGKV1-12 | 0.47 |
| FG.EC.C15orf48.OLFM4 | RP11-357H14.17 | 0.59 | FG.Endth/Cap.PLVAP.FLT1 | CD93 | 1.42 | FG.Plsma/IgA.IGKV3-20.IGKV4-1 | TNFRSF17 | 1.01 |
| FG.EC.C15orf48.OLFM4 | PDZK1IP1 | 0.65 | FG.Endth/Cap.PLVAP.FLT1 | TSPAN7 | 1.41 | FG.Plsma/IgA.IGKV4-1.IGKV1-5 | IGKV1-12 | 2.72 |
| FG.EC.CCL15.SPIB | TSPAN8 | 2.63 | FG.Endth/Cap.PLVAP.FLT1 | COL4A1 | 1.40 | FG.Plsma/IgA.IGKV4-1.IGKV1-5 | IGKV1-5 | 2.46 |
| FG.EC.CCL15.SPIB | OLFM4 | 2.36 | FG.Endth/Cap.PLVAP.FLT1 | NOV | 1.38 | FG.Plsma/IgA.IGKV4-1.IGKV1-5 | IGKV4-1 | 2.17 |
| FG.EC.CCL15.SPIB | GPX2 | 2.11 | FG.Endth/Cap.PLVAP.FLT1 | NQO1 | 1.37 | FG.Plsma/IgA.IGKV4-1.IGKV1-5 | IGKV3-11 | 1.67 |
| FG.EC.CCL15.SPIB | AGR2 | 1.62 | FG.Endth/Cap.PLVAP.FLT1 | LDB2 | 1.37 | FG.Plsma/IgA.IGKV4-1.IGKV1-5 | IGHV3-23 | 0.81 |
| FG.EC.CCL15.SPIB | SLC12A2 | 1.61 | FG.Endth/Cap.PLVAP.FLT1 | EMP2 | 1.37 | FG.Plsma/IgA.IGLC3.IGLC2 | IGLC3 | 4.15 |
| FG.EC.CCL15.SPIB | MGST1 | 1.60 | FG.Endth/Cap.PLVAP.FLT1 | KANK3 | 1.36 | FG.Plsma/IgA.IGLC3.IGLC2 | IGLC2 | 4.15 |
| FG.EC.CCL15.SPIB | CCL15 | 1.57 | FG.Endth/Cap.PLVAP.FLT1 | TINAGL1 | 1.34 | FG.Plsma/IgA.IGLC3.IGLC2 | IGHA1 | 2.69 |
| FG.EC.CCL15.SPIB | KLF5 | 1.47 | FG.Endth/Cap.PLVAP.FLT1 | COL4A2 | 1.32 | FG.Plsma/IgA.IGLC3.IGLC2 | IGHA2 | 2.59 |
| FG.EC.CCL15.SPIB | EDN1 | 1.46 | FG.Endth/Cap.PLVAP.FLT1 | TMEM204 | 1.31 | FG.Plsma/IgA.IGLC3.IGLC2 | JCHAIN | 2.42 |
| FG.EC.CCL15.SPIB | PPP1R1B | 1.26 | FG.Endth/Cap.PLVAP.FLT1 | ITGA1 | 1.31 | FG.Plsma/IgA.IGLC3.IGLC2 | MZB1 | 1.82 |
| FG.EC.CCL15.SPIB | AKR1C3 | 1.25 | FG.Endth/Cap.PLVAP.FLT1 | MMRN2 | 1.30 | FG.Plsma/IgA.IGLC3.IGLC2 | SSR4 | 1.64 |
| FG.EC.CCL15.SPIB | CDX1 | 1.24 | FG.Endth/Cap.PLVAP.FLT1 | SLC14A1 | 1.29 | FG.Plsma/IgA.IGLC3.IGLC2 | DERL3 | 1.49 |
| FG.EC.CCL15.SPIB | TFF3 | 1.15 | FG.Endth/Cap.PLVAP.FLT1 | SLCO2A1 | 1.29 | FG.Plsma/IgA.IGLC3.IGLC2 | IGLV6-57 | 1.47 |
| FG.EC.CCL15.SPIB | STARD10 | 1.09 | FG.Endth/Cap.PLVAP.FLT1 | HYAL2 | 1.29 | FG.Plsma/IgA.IGLC3.IGLC2 | AL928768.3 | 1.39 |
| FG.EC.CCL15.SPIB | FERMT1 | 0.89 | FG.Endth/Cap.PLVAP.FLT1 | NOSTRIN | 1.27 | FG.Plsma/IgA.IGLC3.IGLC2 | FKBP11 | 1.28 |
| FG.EC.CCL15.SPIB | KCNJ13 | 0.83 | FG.Endth/Cap.PLVAP.FLT1 | LIMCH1 | 1.26 | FG.Plsma/IgA.IGLC3.IGLC2 | TNFRSF17 | 1.21 |
| FG.EC.CCL15.SPIB | RP11-357H14.17 | 0.81 | FG.Endth/Cap.PLVAP.FLT1 | ACVRL1 | 1.25 | FG.Plsma/IgA.IGLC3.IGLC2 | HSP90B1 | 1.12 |
| FG.EC.CCL15.SPIB | AQP1 | 0.74 | FG.Endth/Cap.PLVAP.FLT1 | ADGRF5 | 1.24 | FG.Plsma/IgA.IGLC3.IGLC2 | SEC11C | 1.10 |
| FG.EC.CCL15.SPIB | TRABD2A | 0.67 | FG.Endth/Cap.PLVAP.FLT1 | LPAR6 | 1.23 | FG.Plsma/IgA.IGLC3.IGLC2 | JUN | 1.03 |
| FG.EC.CCL15.SPIB | SERPINB5 | 0.64 | FG.Endth/Cap.PLVAP.FLT1 | PRKCDBP | 1.23 | FG.Plsma/IgA.IGLC3.IGLC2 | XBP1 | 1.01 |
| FG.EC.CCL15.SPIB | OSR2 | 0.58 | FG.Endth/Cap.PLVAP.FLT1 | NKX2-3 | 1.19 | FG.Plsma/IgA.IGLC3.IGLC2 | PRDX4 | 0.86 |
| FG.EC.CCL15.SPIB | CSRP2 | 0.55 | FG.Endth/Cap.PLVAP.FLT1 | KDR | 1.19 | FG.Plsma/IgA.IGLC3.IGLC2 | HERPUD1 | 0.85 |
| FG.EC.CCL15.SPIB | HOXB9 | 0.53 | FG.Endth/Cap.PLVAP.FLT1 | FAM167B | 1.19 | FG.Plsma/IgA.IGLC3.IGLC2 | PDIA4 | 0.83 |
| FG.EC.CCL15.SPIB | NTRK2 | 0.50 | FG.Endth/Cap.PLVAP.FLT1 | MYCT1 | 1.17 | FG.Plsma/IgA.IGLC3.IGLC2 | DNAJB9 | 0.82 |
| FG.EC.CCL15.SPIB | ANKRD22 | 0.50 | FG.Endth/Cap.PLVAP.FLT1 | SYNPO | 1.17 | FG.Plsma/IgA.IGLC3.IGLC2 | FKBP2 | 0.81 |
| FG.EC.CCL15.SPIB | CHDH | 0.47 | FG.Endth/Cap.PLVAP.FLT1 | NRP1 | 1.15 | FG.Plsma/IgA.IGLC3.IGLC2 | VIMP | 0.78 |
| FG.EC.CCL15.SPIB | NOXO1 | 0.42 | FG.Endth/Cap.PLVAP.FLT1 | PALMD | 1.15 | FG.Plsma/IgA.IGLC3.IGLC2 | ITM2C | 0.77 |
| FG.EC.CCL15.SPIB | RP11-519G16.5 | 0.40 | FG.Endth/Cap.PLVAP.FLT1 | WWTR1 | 1.12 | FG.Plsma/IgA.IGLC3.IGLC2 | SPCS2 | 0.76 |
| FG.EC.CCL15.SPIB | SDR16C5 | 0.36 | FG.Endth/Cap.PLVAP.FLT1 | APLNR | 1.12 | FG.Plsma/IgA.IGLC3.IGLC2 | MANF | 0.71 |
| FG.EC.CCL15.SPIB | GABRP | 0.35 | FG.Endth/Cap.PLVAP.FLT1 | GPRC5B | 1.07 | FG.Plsma/IgA.IGLC3.IGLC2 | UBE2J1 | 0.68 |
| FG.EC.CCL15.SPIB | GJB3 | 0.32 | FG.Endth/Cap.PLVAP.FLT1 | SOX17 | 1.07 | FG.Plsma/IgA.IGLC3.IGLC2 | SSR3 | 0.67 |
| FG.EC.CCL15.SPIB | LAMC2 | 0.30 | FG.Endth/Cap.PLVAP.FLT1 | FAM110D | 1.05 | FG.Plsma/IgA.IGLC3.IGLC2 | SDF2L1 | 0.63 |
| FG.EC.CCL15.SPIB | KIAA1324 | 0.67 | FG.Endth/Cap.PLVAP.FLT1 | IL3RA | 1.03 | FG.Plsma/IgA.IGLC3.IGLC2 | CRELD2 | 0.60 |
| FG.EC.CCL15.SPIB | FXYD3 | 0.99 | FG.Endth/Cap.PLVAP.FLT1 | HEG1 | 1.03 | FG.Plsma/IgA.IGLC3.IGLC2 | HSPA5 | 0.58 |
| FG.EC.CCL15.SPIB | HOXA9 | 0.41 | FG.Endth/Cap.PLVAP.FLT1 | CDH5 | 1.02 | FG.Plsma/IgA.IGLC3.IGLC2 | SPCS1 | 0.58 |
| FG.EC.CCL15.SPIB | DSC2 | 0.86 | FG.Endth/Cap.PLVAP.FLT1 | LIMS2 | 1.01 | FG.Plsma/IgA.IGLC3.IGLC2 | MYDGF | 0.58 |
| FG.EC.CCL15.SPIB | CCL25 | 2.20 | FG.Endth/Cap.PLVAP.FLT1 | CALD1 | 1.00 | FG.Plsma/IgA.IGLC3.IGLC2 | PDIA6 | 0.55 |
| FG.EC.DMBT1.ADH1C | DMBT1 | 2.33 | FG.Endth/Cap.PLVAP.FLT1 | NES | 1.00 | FG.Plsma/IgA.IGLC3.IGLC2 | SPCS3 | 0.53 |
| FG.EC.DMBT1.ADH1C | PPP1R1B | 0.94 | FG.Endth/Cap.PLVAP.FLT1 | TMEM47 | 0.97 | FG.Plsma/IgA.IGLC3.IGLC2 | SDC1 | 0.52 |
| FG.EC.DMBT1.ADH1C | GOLM1 | 0.89 | FG.Endth/Cap.PLVAP.FLT1 | SNCG | 0.97 | FG.Plsma/IgA.IGLC3.IGLC2 | DUSP5 | 0.49 |
| FG.EC.DMBT1.ADH1C | OTC | 0.88 | FG.Endth/Cap.PLVAP.FLT1 | FAM107A | 0.96 | FG.Plsma/IgA.IGLC3.IGLC2 | MEI1 | 0.46 |
| FG.EC.DMBT1.ADH1C.1 | GSTA1 | 2.53 | FG.Endth/Cap.PLVAP.FLT1 | TIE1 | 0.94 | FG.Plsma/IgA.IGLC3.IGLC2 | CCR10 | 0.45 |
| FG.EC.DMBT1.ADH1C.1 | REG1A | 2.43 | FG.Endth/Cap.PLVAP.FLT1 | NUAK1 | 0.90 | FG.Plsma/IgA.IGLC3.IGLC2 | GNG7 | 0.44 |
| FG.EC.DMBT1.ADH1C.1 | PIGR | 2.38 | FG.Endth/Cap.PLVAP.FLT1 | ROBO4 | 0.87 | FG.Plsma/IgA.IGLC3.IGLC2 | CHPF | 0.43 |
| FG.EC.DMBT1.ADH1C.1 | EPCAM | 2.12 | FG.Endth/Cap.PLVAP.FLT1 | RASIP1 | 0.87 | FG.Plsma/IgA.IGLC3.IGLC2 | ANKRD28 | 0.43 |
| FG.EC.DMBT1.ADH1C.1 | LGALS4 | 2.11 | FG.Endth/Cap.PLVAP.FLT1 | SOX18 | 0.83 | FG.Plsma/IgA.IGLC3.IGLC2 | SLAMF7 | 0.42 |
| FG.EC.DMBT1.ADH1C.1 | ADH1C | 2.11 | FG.Endth/Cap.PLVAP.FLT1 | RAI14 | 0.81 | FG.Plsma/IgA.IGLC3.IGLC2 | PLPP5 | 0.42 |
| FG.EC.DMBT1.ADH1C.1 | MT1G | 2.08 | FG.Endth/Cap.PLVAP.FLT1 | ATOH8 | 0.79 | FG.Plsma/IgA.IGLC3.IGLC2 | NUCB2 | 0.42 |
| FG.EC.DMBT1.ADH1C.1 | CCL25 | 2.08 | FG.Endth/Cap.PLVAP.FLT1 | HSPA12B | 0.77 | FG.Plsma/IgA.IGLC3.IGLC2 | TRAM1 | 0.41 |
| FG.EC.DMBT1.ADH1C.1 | FABP1 | 2.03 | FG.Endth/Cap.PLVAP.FLT1 | TEK | 0.75 | FG.Plsma/IgA.IGLC3.IGLC2 | FCRL5 | 0.39 |
| FG.EC.DMBT1.ADH1C.1 | TSPAN8 | 2.03 | FG.Endth/Cap.PLVAP.FLT1 | GALNT18 | 0.75 | FG.Plsma/IgA.IGLC3.IGLC2 | DNAJC3 | 0.38 |
| FG.EC.DMBT1.ADH1C.1 | KRT8 | 2.02 | FG.Endth/Cap.PLVAP.FLT1 | TMTC1 | 0.74 | FG.Plsma/IgA.IGLC3.IGLC2 | TXNDC15 | 0.38 |
| FG.EC.DMBT1.ADH1C.1 | DMBT1 | 1.96 | FG.Endth/Cap.PLVAP.FLT1 | CXorf36 | 0.73 | FG.Plsma/IgA.IGLC3.IGLC2 | RPN2 | 0.37 |
| FG.EC.DMBT1.ADH1C.1 | RBP2 | 1.92 | FG.Endth/Cap.PLVAP.FLT1 | ADCY4 | 0.71 | FG.Plsma/IgA.IGLC3.IGLC2 | HM13 | 0.37 |
| FG.EC.DMBT1.ADH1C.1 | CES2 | 1.76 | FG.Endth/Cap.PLVAP.FLT1 | ERG | 0.70 | FG.Plsma/IgA.IGLC3.IGLC2 | TXNDC11 | 0.37 |
| FG.EC.DMBT1.ADH1C.1 | KRT18 | 1.70 | FG.Endth/Cap.PLVAP.FLT1 | PTPRM | 0.69 | FG.Plsma/IgA.IGLC3.IGLC2 | UAP1 | 0.36 |
| FG.EC.DMBT1.ADH1C.1 | CBR1 | 1.63 | FG.Endth/Cap.PLVAP.FLT1 | ARHGEF15 | 0.68 | FG.Plsma/IgA.IGLC3.IGLC2 | CCPG1 | 0.36 |
| FG.EC.DMBT1.ADH1C.1 | MT1E | 1.61 | FG.Endth/Cap.PLVAP.FLT1 | PLPP3 | 1.26 | FG.Plsma/IgA.IGLC3.IGLC2 | TMED9 | 0.36 |
| FG.EC.DMBT1.ADH1C.1 | CLDN3 | 1.60 | FG.Endth/Cap.PLVAP.FLT1 | BTNL9 | 0.85 | FG.Plsma/IgA.IGLC3.IGLC2 | KRTCAP2 | 0.35 |
| FG.EC.DMBT1.ADH1C.1 | KRT19 | 1.58 | FG.Endth/Cap.PLVAP.FLT1 | PLCB1 | 0.77 | FG.Plsma/IgA.IGLC3.IGLC2 | FAM46C | 0.35 |
| FG.EC.DMBT1.ADH1C.1 | AGR2 | 1.58 | FG.Endth/Cap.PLVAP.FLT1 | IQCK | 0.84 | FG.Plsma/IgA.IGLC3.IGLC2 | LMAN1 | 0.35 |
| FG.EC.DMBT1.ADH1C.1 | MT1H | 1.58 | FG.Endth/Cap.PLVAP.FLT1 | SHE | 0.69 | FG.Plsma/IgA.IGLC3.IGLC2 | SEL1L | 0.35 |
| FG.EC.DMBT1.ADH1C.1 | CLDN4 | 1.57 | FG.Endth/Cap.PLVAP.FLT1 | CLIC2 | 1.05 | FG.Plsma/IgA.IGLC3.IGLC2 | AMPD1 | 0.35 |
| FG.EC.DMBT1.ADH1C.1 | PTGR1 | 1.56 | FG.Endth/Cap.PLVAP.FLT1 | PCDH17 | 0.74 | FG.Plsma/IgA.IGLC3.IGLC2 | LMAN2 | 0.35 |
| FG.EC.DMBT1.ADH1C.1 | AKR1B10 | 1.50 | FG.Endth/Cap.PLVAP.FLT1 | MGLL | 1.28 | FG.Plsma/IgA.IGLC3.IGLC2 | RPN1 | 0.34 |
| FG.EC.DMBT1.ADH1C.1 | MT2A | 1.49 | FG.Endth/Lymph.CCL21.LYVE1 | CCL21 | 4.85 | FG.Plsma/IgA.IGLC3.IGLC2 | PABPC4 | 0.33 |
| FG.EC.DMBT1.ADH1C.1 | ELF3 | 1.48 | FG.Endth/Lymph.CCL21.LYVE1 | GNG11 | 3.05 | FG.Plsma/IgA.IGLC3.IGLC2 | EAF2 | 0.33 |
| FG.EC.DMBT1.ADH1C.1 | AKR1C3 | 1.48 | FG.Endth/Lymph.CCL21.LYVE1 | TFF3 | 2.98 | FG.Plsma/IgA.IGLC3.IGLC2 | ERLEC1 | 0.33 |
| FG.EC.DMBT1.ADH1C.1 | TXN | 1.47 | FG.Endth/Lymph.CCL21.LYVE1 | LYVE1 | 2.71 | FG.Plsma/IgA.IGLC3.IGLC2 | SELM | 0.32 |
| FG.EC.DMBT1.ADH1C.1 | CMBL | 1.46 | FG.Endth/Lymph.CCL21.LYVE1 | SDPR | 2.64 | FG.Plsma/IgA.IGLC3.IGLC2 | GMPPB | 0.31 |
| FG.EC.DMBT1.ADH1C.1 | MT1X | 1.44 | FG.Endth/Lymph.CCL21.LYVE1 | EFEMP1 | 2.48 | FG.Plsma/IgA.IGLC3.IGLC2 | MANEA | 0.30 |
| FG.EC.DMBT1.ADH1C.1 | GSTA2 | 1.43 | FG.Endth/Lymph.CCL21.LYVE1 | CLDN5 | 2.29 | FG.Plsma/IgA.IGLC3.IGLC2 | SPAG4 | 0.29 |
| FG.EC.DMBT1.ADH1C.1 | MGST3 | 1.42 | FG.Endth/Lymph.CCL21.LYVE1 | PCAT19 | 2.24 | FG.Plsma/IgA.IGLC3.IGLC2 | PDK1 | 0.27 |
| FG.EC.DMBT1.ADH1C.1 | SMIM24 | 1.41 | FG.Endth/Lymph.CCL21.LYVE1 | FXYD6 | 2.23 | FG.Plsma/IgA.IGLC3.IGLC2 | ALG5 | 0.26 |
| FG.EC.DMBT1.ADH1C.1 | LGALS3 | 1.37 | FG.Endth/Lymph.CCL21.LYVE1 | ECSCR-1 | 2.23 | FG.Plsma/IgA.IGLC3.IGLC2 | DDOST | 0.26 |
| FG.EC.DMBT1.ADH1C.1 | ID1 | 1.37 | FG.Endth/Lymph.CCL21.LYVE1 | MMRN1 | 2.15 | FG.Plsma/IgA.IGLC3.IGLC2 | SRPRB | 0.25 |
| FG.EC.DMBT1.ADH1C.1 | ANPEP | 1.37 | FG.Endth/Lymph.CCL21.LYVE1 | PROX1 | 1.92 | FG.Plsma/IgA.IGLC3.IGLC2 | DNAJC1 | 0.25 |
| FG.EC.DMBT1.ADH1C.1 | CLDN7 | 1.36 | FG.Endth/Lymph.CCL21.LYVE1 | NRP2 | 1.76 | FG.Plsma/IgA.IGLL5.IGLV2-14 | IGLL5 | 4.63 |
| FG.EC.DMBT1.ADH1C.1 | AGR3 | 1.33 | FG.Endth/Lymph.CCL21.LYVE1 | EGFL7 | 1.64 | FG.Plsma/IgA.IGLL5.IGLV2-14 | IGHA2 | 2.95 |
| FG.EC.DMBT1.ADH1C.1 | ATP5G3 | 1.32 | FG.Endth/Lymph.CCL21.LYVE1 | CCL14 | 1.51 | FG.Plsma/IgA.IGLL5.IGLV2-14 | IGHA1 | 2.92 |
| FG.EC.DMBT1.ADH1C.1 | OAT | 1.31 | FG.Endth/Lymph.CCL21.LYVE1 | FLT4 | 1.19 | FG.Plsma/IgA.IGLL5.IGLV2-14 | JCHAIN | 2.74 |
| FG.EC.DMBT1.ADH1C.1 | CYB5A | 1.30 | FG.Endth/Lymph.CCL21.LYVE1 | PARD6G | 1.13 | FG.Plsma/IgA.IGLL5.IGLV2-14 | MZB1 | 2.10 |
| FG.EC.DMBT1.ADH1C.1 | MT1F | 1.30 | FG.Endth/Lymph.CCL21.LYVE1 | RELN | 1.09 | FG.Plsma/IgA.IGLL5.IGLV2-14 | SSR4 | 2.01 |
| FG.EC.DMBT1.ADH1C.1 | PHGR1 | 1.29 | FG.Endth/Lymph.CCL21.LYVE1 | ADGRG3 | 1.09 | FG.Plsma/IgA.IGLL5.IGLV2-14 | DERL3 | 1.78 |
| FG.EC.DMBT1.ADH1C.1 | CLDN15 | 1.28 | FG.Endth/Lymph.CCL21.LYVE1 | TBX1 | 0.72 | FG.Plsma/IgA.IGLL5.IGLV2-14 | AL928768.3 | 1.69 |
| FG.EC.DMBT1.ADH1C.1 | DHRS11 | 1.26 | FG.Endth/Lymph.CCL21.LYVE1 | RP11-322E11.5 | 0.68 | FG.Plsma/IgA.IGLL5.IGLV2-14 | FKBP11 | 1.61 |
| FG.EC.DMBT1.ADH1C.1 | PEBP1 | 1.26 | FG.Endth/Lymph.CCL21.LYVE1 | TIE1 | 1.23 | FG.Plsma/IgA.IGLL5.IGLV2-14 | TNFRSF17 | 1.60 |
| FG.EC.DMBT1.ADH1C.1 | CPS1 | 1.26 | FG.Endth/Lymph.CCL21.LYVE1 | OMG | 1.10 | FG.Plsma/IgA.IGLL5.IGLV2-14 | IGLV6-57 | 1.43 |
| FG.EC.DMBT1.ADH1C.1 | FABP2 | 1.21 | FG.Endth/Lymph.CCL21.PROX1 | CCL21 | 3.98 | FG.Plsma/IgA.IGLL5.IGLV2-14 | SEC11C | 1.42 |
| FG.EC.DMBT1.ADH1C.1 | PCK1 | 1.20 | FG.Endth/Lymph.CCL21.PROX1 | PROX1 | 2.65 | FG.Plsma/IgA.IGLL5.IGLV2-14 | XBP1 | 1.33 |
| FG.EC.DMBT1.ADH1C.1 | HSD17B2 | 1.19 | FG.Endth/Lymph.CCL21.PROX1 | MMRN1 | 2.32 | FG.Plsma/IgA.IGLL5.IGLV2-14 | PRDX4 | 1.19 |
| FG.EC.DMBT1.ADH1C.1 | S100A14 | 1.19 | FG.Endth/Lymph.CCL21.PROX1 | LYVE1 | 2.27 | FG.Plsma/IgA.IGLL5.IGLV2-14 | SSR3 | 1.04 |
| FG.EC.DMBT1.ADH1C.1 | ALDH1A1 | 1.19 | FG.Endth/Lymph.RP11-136K72.LYVE1 | LYVE1 | 3.87 | FG.Plsma/IgA.IGLL5.IGLV2-14 | CCR10 | 0.57 |
| FG.EC.DMBT1.ADH1C.1 | SI | 1.19 | FG.Endth/Lymph.RP11-136K72.LYVE1 | EFEMP1 | 3.73 | FG.Plsma/IgG.IGHGP.IGHG2 | IGHGP | 3.96 |
| FG.EC.DMBT1.ADH1C.1 | ATP1B1 | 1.17 | FG.Endth/Lymph.RP11-136K72.LYVE1 | CCL21 | 3.66 | FG.Plsma/IgG.IGHGP.IGHG2.1 | IGHG1 | 4.69 |
| FG.EC.DMBT1.ADH1C.1 | FBP1 | 1.16 | FG.Endth/Lymph.RP11-136K72.LYVE1 | MMRN1 | 3.55 | FG.Plsma/IgG.IGHGP.IGHG2.1 | IGHG3 | 4.64 |
| FG.EC.DMBT1.ADH1C.1 | LIMA1 | 1.16 | FG.Endth/Lymph.RP11-136K72.LYVE1 | PROX1 | 2.70 | FG.Plsma/IgG.IGHGP.IGHG2.1 | IGHG4 | 4.25 |
| FG.EC.DMBT1.ADH1C.1 | MGST2 | 1.15 | FG.Endth/Lymph.RP11-136K72.LYVE1 | RP11-136K7.2 | 1.82 | FG.Plsma/IgG.IGHGP.IGHG2.1 | IGHG2 | 4.09 |
| FG.EC.DMBT1.ADH1C.1 | HMGCS2 | 1.13 | FG.Endth/Lymph.RP11-136K72.LYVE1 | RELN | 1.44 | FG.Plsma/IgG.IGHGP.IGHG2.1 | IGHGP | 4.06 |
| FG.EC.DMBT1.ADH1C.1 | KHK | 1.12 | FG.Endth/Lymph.RP11-136K72.LYVE1 | MEDAG | 1.30 | FG.Plsma/IgG.IGHGP.IGHG2.1 | IGKC | 2.94 |
| FG.EC.DMBT1.ADH1C.1 | GATM | 1.11 | FG.Endth/Lymph.RP11-136K72.LYVE1 | FZD9 | 0.70 | FG.Plsma/IgG.IGHGP.IGHG2.1 | MZB1 | 1.75 |
| FG.EC.DMBT1.ADH1C.1 | SUCLG1 | 1.10 | FG.Endth/Lymph.TFPI.LYVE1 | MMRN1 | 2.85 | FG.Plsma/IgG.IGHGP.IGHG2.1 | XBP1 | 1.58 |
| FG.EC.DMBT1.ADH1C.1 | AADAC | 1.09 | FG.Endth/Lymph.TFPI.LYVE1 | LYVE1 | 2.73 | FG.Plsma/IgG.IGHGP.IGHG2.1 | IGKV1-12 | 1.49 |
| FG.EC.DMBT1.ADH1C.1 | RAMP1 | 1.09 | FG.Endth/Lymph.TFPI.LYVE1 | CCL21 | 2.68 | FG.Plsma/IgG.IGHGP.IGHG2.1 | DERL3 | 1.00 |
| FG.EC.DMBT1.ADH1C.1 | FCGRT | 1.09 | FG.Endth/Lymph.TFPI.LYVE1 | PROX1 | 2.39 | FG.Plsma/IgG.IGHGP.IGHG2.2 | IGHG3 | 4.59 |
| FG.EC.DMBT1.ADH1C.1 | FAM3B | 1.08 | FG.Endth/Lymph.TFPI.LYVE1 | RELN | 1.68 | FG.Plsma/IgG.IGHGP.IGHG2.2 | IGHG1 | 4.57 |
| FG.EC.DMBT1.ADH1C.1 | AKR7A3 | 1.08 | FG.Endth/Lymph.TFPI.LYVE1 | CLEC4M | 1.31 | FG.Plsma/IgG.IGHGP.IGHG2.2 | IGHG4 | 4.20 |
| FG.EC.DMBT1.ADH1C.1 | ALDOB | 1.08 | FG.Endth/Lymph.TFPI.LYVE1 | EFEMP1 | 2.51 | FG.Plsma/IgG.IGHGP.IGHG2.2 | IGHG2 | 4.08 |
| FG.EC.DMBT1.ADH1C.1 | UGT2B17 | 1.07 | FG.Endth/Ven.ACKR1.MADCAM1 | ACKR1 | 3.55 | FG.Plsma/IgG.IGHGP.IGHG2.2 | IGHGP | 3.82 |
| FG.EC.DMBT1.ADH1C.1 | DSP | 1.06 | FG.Endth/Ven.ACKR1.MADCAM1 | CLDN5 | 3.30 | FG.Plsma/IgG.IGHGP.IGHG2.2 | MZB1 | 1.99 |
| FG.EC.DMBT1.ADH1C.1 | MGST1 | 1.06 | FG.Endth/Ven.ACKR1.MADCAM1 | SPARCL1 | 3.13 | FG.Plsma/IgG.IGHGP.IGHG2.2 | IGLV6-57 | 1.83 |
| FG.EC.DMBT1.ADH1C.1 | ANXA2 | 1.06 | FG.Endth/Ven.ACKR1.MADCAM1 | IFI27 | 2.86 | FG.Plsma/IgG.IGHGP.IGHG2.2 | DERL3 | 1.34 |
| FG.EC.DMBT1.ADH1C.1 | PPP1R16A | 1.05 | FG.Endth/Ven.ACKR1.MADCAM1 | FABP5 | 2.82 | FG.Plsma/IgM.IGHM.IGLV2-14 | IGHM | 5.94 |
| FG.EC.DMBT1.ADH1C.1 | SMIM22 | 1.04 | FG.Endth/Ven.ACKR1.MADCAM1 | CPE | 2.72 | FG.Plsma/IgM.IGHM.IGLV2-14 | JCHAIN | 2.22 |
| FG.EC.DMBT1.ADH1C.1 | PRSS3 | 1.02 | FG.Endth/Ven.ACKR1.MADCAM1 | IGFBP7 | 2.52 | FG.Plsma/IgM.IGHM.IGLV2-14 | MZB1 | 1.80 |
| FG.EC.DMBT1.ADH1C.1 | KLF5 | 1.02 | FG.Endth/Ven.ACKR1.MADCAM1 | CD320 | 2.40 | FG.Plsma/IgM.IGHM.IGLV2-14 | SSR4 | 1.38 |
| FG.EC.DMBT1.ADH1C.1 | CYC1 | 1.02 | FG.Endth/Ven.ACKR1.MADCAM1 | IGFBP4 | 2.33 | FG.Plsma/IgM.IGHM.IGLV2-14 | TNFRSF17 | 1.17 |
| FG.EC.DMBT1.ADH1C.1 | ATP1A1 | 1.01 | FG.Endth/Ven.ACKR1.MADCAM1 | GNG11 | 2.29 | FG.Plsma/IgM.IGHM.IGLV2-14 | SEC11C | 1.12 |
| FG.EC.DMBT1.ADH1C.1 | ANXA4 | 1.00 | FG.Endth/Ven.ACKR1.MADCAM1 | PLVAP | 2.29 | FG.Plsma/IgM.IGHM.IGLV2-14 | FKBP11 | 1.09 |
| FG.EC.DMBT1.ADH1C.1 | MME | 1.00 | FG.Endth/Ven.ACKR1.MADCAM1 | RAMP2 | 2.28 | FG.Plsma/IgM.IGHM.IGLV2-14 | DERL3 | 1.08 |
| FG.EC.DMBT1.ADH1C.1 | ADIRF | 1.00 | FG.Endth/Ven.ACKR1.MADCAM1 | CLU | 2.23 | FG.Plsma/IgM.IGHM.IGLV2-14 | XBP1 | 1.07 |
| FG.EC.DMBT1.ADH1C.1 | CYSTM1 | 1.00 | FG.Endth/Ven.ACKR1.MADCAM1 | CAV1 | 2.22 | FG.Plsma/IgM.IGHM.IGLV2-14 | HERPUD1 | 0.96 |
| FG.EC.DMBT1.ADH1C.1 | SLC5A1 | 0.99 | FG.Endth/Ven.ACKR1.MADCAM1 | TSPAN7 | 2.21 | FG.Plsma/IgM.IGHM.IGLV2-14 | HSP90B1 | 0.84 |
| FG.EC.DMBT1.ADH1C.1 | CISD3 | 0.99 | FG.Endth/Ven.ACKR1.MADCAM1 | NPC2 | 2.18 | FG.Plsma/IgM.IGHM.IGLV2-14 | PRDX4 | 0.74 |
| FG.EC.DMBT1.ADH1C.1 | SFN | 0.98 | FG.Endth/Ven.ACKR1.MADCAM1 | RAMP3 | 2.16 | FG.Plsma/IgM.IGHM.IGLV2-14 | MANF | 0.73 |
| FG.EC.DMBT1.ADH1C.1 | PHLDA2 | 0.97 | FG.Endth/Ven.ACKR1.MADCAM1 | CRIP2 | 2.12 | FG.Plsma/IgM.IGHM.IGLV2-14 | DNAJB9 | 0.70 |
| FG.EC.DMBT1.ADH1C.1 | MAOA | 0.97 | FG.Endth/Ven.ACKR1.MADCAM1 | JAM2 | 2.06 | FG.Plsma/IgM.IGHM.IGLV2-14 | SSR3 | 0.70 |
| FG.EC.DMBT1.ADH1C.1 | ATP5G1 | 0.96 | FG.Endth/Ven.ACKR1.MADCAM1 | TM4SF1 | 2.06 | FG.Plsma/IgM.IGHM.IGLV2-14 | VIMP | 0.69 |
| FG.EC.DMBT1.ADH1C.1 | MPST | 0.96 | FG.Endth/Ven.ACKR1.MADCAM1 | CTGF | 2.03 | FG.Plsma/IgM.IGHM.IGLV2-14 | PDIA4 | 0.68 |
| FG.EC.DMBT1.ADH1C.1 | C19orf33 | 0.96 | FG.Endth/Ven.ACKR1.MADCAM1 | RNASE1 | 1.98 | FG.Plsma/IgM.IGHM.IGLV2-14 | SDF2L1 | 0.66 |
| FG.EC.DMBT1.ADH1C.1 | S100A16 | 0.96 | FG.Endth/Ven.ACKR1.MADCAM1 | PECAM1 | 1.96 | FG.Plsma/IgM.IGHM.IGLV2-14 | CRELD2 | 0.63 |
| FG.EC.DMBT1.ADH1C.1 | EGR1 | 0.94 | FG.Endth/Ven.ACKR1.MADCAM1 | LY6E | 1.96 | FG.Plsma/IgM.IGHM.IGLV2-14 | ANKRD28 | 0.62 |
| FG.EC.DMBT1.ADH1C.1 | CRYL1 | 0.92 | FG.Endth/Ven.ACKR1.MADCAM1 | LIFR | 1.96 | FG.Plsma/IgM.IGHM.IGLV2-14 | UBE2J1 | 0.62 |
| FG.EC.DMBT1.ADH1C.1 | SLC26A3 | 0.91 | FG.Endth/Ven.ACKR1.MADCAM1 | MADCAM1 | 1.95 | FG.Plsma/IgM.IGHM.IGLV2-14 | SPCS3 | 0.52 |
| FG.EC.DMBT1.ADH1C.1 | TKFC | 0.90 | FG.Endth/Ven.ACKR1.MADCAM1 | IFITM3 | 1.95 | FG.Plsma/IgM.IGHM.IGLV2-14 | CCR10 | 0.49 |
| FG.EC.DMBT1.ADH1C.1 | AMN | 0.90 | FG.Endth/Ven.ACKR1.MADCAM1 | PCAT19 | 1.95 | FG.Plsma/IgM.IGHM.IGLV2-14 | FAM46C | 0.46 |
| FG.EC.DMBT1.ADH1C.1 | HPGD | 0.89 | FG.Endth/Ven.ACKR1.MADCAM1 | TNFSF10 | 1.93 | FG.Plsma/IgM.IGHM.IGLV2-14 | FCRL5 | 0.43 |
| FG.EC.DMBT1.ADH1C.1 | HEBP1 | 0.89 | FG.Endth/Ven.ACKR1.MADCAM1 | VWF | 1.90 | FG.Plsma/IgM.IGHM.IGLV2-14 | SLAMF7 | 0.41 |
| FG.EC.DMBT1.ADH1C.1 | DDT | 0.88 | FG.Endth/Ven.ACKR1.MADCAM1 | CALCRL | 1.81 | FG.Plsma/IgM.IGHM.IGLV2-14 | FKBP2 | 0.66 |
| FG.EC.DMBT1.ADH1C.1 | CHP2 | 0.88 | FG.Endth/Ven.ACKR1.MADCAM1 | TGFBR2 | 1.79 | FG.Plsma/IgM.IGHM.IGLV2-14 | SPCS2 | 0.72 |
| FG.EC.DMBT1.ADH1C.1 | SLC39A5 | 0.87 | FG.Endth/Ven.ACKR1.MADCAM1 | CTNNAL1 | 1.78 | FG.Prolif/EC.UBE2C.TOP2A | REG1A | 3.02 |
| FG.EC.DMBT1.ADH1C.1 | TMEM45B | 0.87 | FG.Endth/Ven.ACKR1.MADCAM1 | PTRF | 1.76 | FG.Prolif/EC.UBE2C.TOP2A | OLFM4 | 2.72 |
| FG.EC.DMBT1.ADH1C.1 | HSPA1B | 0.87 | FG.Endth/Ven.ACKR1.MADCAM1 | A2M | 1.76 | FG.Prolif/EC.UBE2C.TOP2A | MT1G | 2.38 |
| FG.EC.DMBT1.ADH1C.1 | SPINK1 | 0.87 | FG.Endth/Ven.ACKR1.MADCAM1 | APLNR | 1.72 | FG.Prolif/EC.UBE2C.TOP2A | MT1E | 2.25 |
| FG.EC.DMBT1.ADH1C.1 | PBLD | 0.87 | FG.Endth/Ven.ACKR1.MADCAM1 | CD59 | 1.71 | FG.Prolif/EC.UBE2C.TOP2A | MT2A | 2.13 |
| FG.EC.DMBT1.ADH1C.1 | ETHE1 | 0.86 | FG.Endth/Ven.ACKR1.MADCAM1 | BCAM | 1.69 | FG.Prolif/EC.UBE2C.TOP2A | AGR2 | 2.11 |
| FG.EC.DMBT1.ADH1C.1 | MISP | 0.86 | FG.Endth/Ven.ACKR1.MADCAM1 | EGFL7 | 1.67 | FG.Prolif/EC.UBE2C.TOP2A | CCL25 | 2.09 |
| FG.EC.DMBT1.ADH1C.1 | SULT1A1 | 0.85 | FG.Endth/Ven.ACKR1.MADCAM1 | CNN3 | 1.67 | FG.Prolif/EC.UBE2C.TOP2A | SPINK1 | 1.96 |
| FG.EC.DMBT1.ADH1C.1 | MTTP | 0.84 | FG.Endth/Ven.ACKR1.MADCAM1 | NPDC1 | 1.65 | FG.Prolif/EC.UBE2C.TOP2A | EPCAM | 1.95 |
| FG.EC.DMBT1.ADH1C.1 | SDCBP2 | 0.84 | FG.Endth/Ven.ACKR1.MADCAM1 | MGP | 1.65 | FG.Prolif/EC.UBE2C.TOP2A | C15orf48 | 1.92 |
| FG.EC.DMBT1.ADH1C.1 | EDN1 | 0.83 | FG.Endth/Ven.ACKR1.MADCAM1 | ENPP2 | 1.65 | FG.Prolif/EC.UBE2C.TOP2A | PIGR | 1.86 |
| FG.EC.DMBT1.ADH1C.1 | SERPINB6 | 0.83 | FG.Endth/Ven.ACKR1.MADCAM1 | NNMT | 1.63 | FG.Prolif/EC.UBE2C.TOP2A | LGALS4 | 1.85 |
| FG.EC.DMBT1.ADH1C.1 | TSPO | 0.82 | FG.Endth/Ven.ACKR1.MADCAM1 | ADGRG6 | 1.61 | FG.Prolif/EC.UBE2C.TOP2A | MT1H | 1.79 |
| FG.EC.DMBT1.ADH1C.1 | OTC | 0.82 | FG.Endth/Ven.ACKR1.MADCAM1 | IL33 | 1.61 | FG.Prolif/EC.UBE2C.TOP2A | DMBT1 | 1.76 |
| FG.EC.DMBT1.ADH1C.1 | DNASE1 | 0.81 | FG.Endth/Ven.ACKR1.MADCAM1 | ENG | 1.61 | FG.Prolif/EC.UBE2C.TOP2A | MT1X | 1.76 |
| FG.EC.DMBT1.ADH1C.1 | CA2 | 0.80 | FG.Endth/Ven.ACKR1.MADCAM1 | PLAT | 1.60 | FG.Prolif/EC.UBE2C.TOP2A | KRT18 | 1.75 |
| FG.EC.DMBT1.ADH1C.1 | HBEGF | 0.80 | FG.Endth/Ven.ACKR1.MADCAM1 | TIMP1 | 1.59 | FG.Prolif/EC.UBE2C.TOP2A | KRT8 | 1.74 |
| FG.EC.DMBT1.ADH1C.1 | VDAC1 | 0.80 | FG.Endth/Ven.ACKR1.MADCAM1 | CLEC14A | 1.54 | FG.Prolif/EC.UBE2C.TOP2A | TSPAN8 | 1.68 |
| FG.EC.DMBT1.ADH1C.1 | VIL1 | 0.79 | FG.Endth/Ven.ACKR1.MADCAM1 | SNCG | 1.48 | FG.Prolif/EC.UBE2C.TOP2A | TUBB4B | 1.66 |
| FG.EC.DMBT1.ADH1C.1 | CDH17 | 0.78 | FG.Endth/Ven.ACKR1.MADCAM1 | ECSCR-1 | 1.47 | FG.Prolif/EC.UBE2C.TOP2A | MT1F | 1.66 |
| FG.EC.DMBT1.ADH1C.1 | COA3 | 0.78 | FG.Endth/Ven.ACKR1.MADCAM1 | ESAM | 1.41 | FG.Prolif/EC.UBE2C.TOP2A | UBE2C | 1.62 |
| FG.EC.DMBT1.ADH1C.1 | LGALS3BP | 0.77 | FG.Endth/Ven.ACKR1.MADCAM1 | FAM167B | 1.39 | FG.Prolif/EC.UBE2C.TOP2A | MGST1 | 1.62 |
| FG.EC.DMBT1.ADH1C.1 | GSTM4 | 0.77 | FG.Endth/Ven.ACKR1.MADCAM1 | KCTD12 | 1.38 | FG.Prolif/EC.UBE2C.TOP2A | TXN | 1.58 |
| FG.EC.DMBT1.ADH1C.1 | UQCRC1 | 0.77 | FG.Endth/Ven.ACKR1.MADCAM1 | MTUS1 | 1.38 | FG.Prolif/EC.UBE2C.TOP2A | GPX2 | 1.48 |
| FG.EC.DMBT1.ADH1C.1 | MALL | 0.77 | FG.Endth/Ven.ACKR1.MADCAM1 | ADIRF | 1.37 | FG.Prolif/EC.UBE2C.TOP2A | CLDN3 | 1.43 |
| FG.EC.DMBT1.ADH1C.1 | PRAP1 | 0.77 | FG.Endth/Ven.ACKR1.MADCAM1 | CD34 | 1.37 | FG.Prolif/EC.UBE2C.TOP2A | CLDN4 | 1.42 |
| FG.EC.DMBT1.ADH1C.1 | CCND2 | 0.76 | FG.Endth/Ven.ACKR1.MADCAM1 | S100A13 | 1.37 | FG.Prolif/EC.UBE2C.TOP2A | HMGB2 | 1.41 |
| FG.EC.DMBT1.ADH1C.1 | LIPH | 0.76 | FG.Endth/Ven.ACKR1.MADCAM1 | CD93 | 1.36 | FG.Prolif/EC.UBE2C.TOP2A | KRT19 | 1.41 |
| FG.EC.DMBT1.ADH1C.1 | REEP6 | 0.76 | FG.Endth/Ven.ACKR1.MADCAM1 | SORBS2 | 1.36 | FG.Prolif/EC.UBE2C.TOP2A | AGR3 | 1.39 |
| FG.EC.DMBT1.ADH1C.1 | GOLM1 | 0.76 | FG.Endth/Ven.ACKR1.MADCAM1 | HYAL2 | 1.36 | FG.Prolif/EC.UBE2C.TOP2A | PHGR1 | 1.39 |
| FG.EC.DMBT1.ADH1C.1 | TIMM13 | 0.74 | FG.Endth/Ven.ACKR1.MADCAM1 | RBP5 | 1.34 | FG.Prolif/EC.UBE2C.TOP2A | ADH1C | 1.38 |
| FG.EC.DMBT1.ADH1C.1 | CYP3A5 | 0.74 | FG.Endth/Ven.ACKR1.MADCAM1 | IL6ST | 1.34 | FG.Prolif/EC.UBE2C.TOP2A | CKS2 | 1.37 |
| FG.EC.DMBT1.ADH1C.1 | VMP1 | 0.74 | FG.Endth/Ven.ACKR1.MADCAM1 | LIMCH1 | 1.34 | FG.Prolif/EC.UBE2C.TOP2A | TOP2A | 1.37 |
| FG.EC.DMBT1.ADH1C.1 | ATF3 | 0.73 | FG.Endth/Ven.ACKR1.MADCAM1 | SDPR | 1.34 | FG.Prolif/EC.UBE2C.TOP2A | ID1 | 1.34 |
| FG.EC.DMBT1.ADH1C.1 | UGT2A3 | 0.73 | FG.Endth/Ven.ACKR1.MADCAM1 | EMCN | 1.34 | FG.Prolif/EC.UBE2C.TOP2A | CLDN7 | 1.30 |
| FG.EC.DMBT1.ADH1C.1 | PPP1R1B | 0.72 | FG.Endth/Ven.ACKR1.MADCAM1 | CCL14 | 1.34 | FG.Prolif/EC.UBE2C.TOP2A | PTTG1 | 1.30 |
| FG.EC.DMBT1.ADH1C.1 | MLEC | 0.72 | FG.Endth/Ven.ACKR1.MADCAM1 | PTPRB | 1.32 | FG.Prolif/EC.UBE2C.TOP2A | STMN1 | 1.28 |
| FG.EC.DMBT1.ADH1C.1 | PYCARD | 0.72 | FG.Endth/Ven.ACKR1.MADCAM1 | ARHGAP29 | 1.32 | FG.Prolif/EC.UBE2C.TOP2A | SLC12A2 | 1.28 |
| FG.EC.DMBT1.ADH1C.1 | MRPL12 | 0.72 | FG.Endth/Ven.ACKR1.MADCAM1 | EPAS1 | 1.32 | FG.Prolif/EC.UBE2C.TOP2A | MKI67 | 1.28 |
| FG.EC.DMBT1.ADH1C.1 | CDX1 | 0.71 | FG.Endth/Ven.ACKR1.MADCAM1 | NFIB | 1.31 | FG.Prolif/EC.UBE2C.TOP2A | KLF5 | 1.24 |
| FG.EC.DMBT1.ADH1C.1 | DDC | 0.71 | FG.Endth/Ven.ACKR1.MADCAM1 | CYYR1 | 1.31 | FG.Prolif/EC.UBE2C.TOP2A | CENPF | 1.24 |
| FG.EC.DMBT1.ADH1C.1 | SLC25A37 | 0.71 | FG.Endth/Ven.ACKR1.MADCAM1 | GIMAP4 | 1.29 | FG.Prolif/EC.UBE2C.TOP2A | TUBA1C | 1.22 |
| FG.EC.DMBT1.ADH1C.1 | TM4SF5 | 0.71 | FG.Endth/Ven.ACKR1.MADCAM1 | NR2F2 | 1.27 | FG.Prolif/EC.UBE2C.TOP2A | MLEC | 1.22 |
| FG.EC.DMBT1.ADH1C.1 | EIF6 | 0.71 | FG.Endth/Ven.ACKR1.MADCAM1 | STOM | 1.26 | FG.Prolif/EC.UBE2C.TOP2A | ELF3 | 1.21 |
| FG.EC.DMBT1.ADH1C.1 | CHPT1 | 0.70 | FG.Endth/Ven.ACKR1.MADCAM1 | PRSS23 | 1.24 | FG.Prolif/EC.UBE2C.TOP2A | GSTA1 | 1.21 |
| FG.EC.DMBT1.ADH1C.1 | FAM195A | 0.70 | FG.Endth/Ven.ACKR1.MADCAM1 | TJP1 | 1.24 | FG.Prolif/EC.UBE2C.TOP2A | MT1M | 1.16 |
| FG.EC.DMBT1.ADH1C.1 | KRTCAP3 | 0.70 | FG.Endth/Ven.ACKR1.MADCAM1 | CFI | 1.22 | FG.Prolif/EC.UBE2C.TOP2A | CCNB1 | 1.15 |
| FG.EC.DMBT1.ADH1C.1 | ANXA13 | 0.70 | FG.Endth/Ven.ACKR1.MADCAM1 | POSTN | 1.20 | FG.Prolif/EC.UBE2C.TOP2A | NUSAP1 | 1.10 |
| FG.EC.DMBT1.ADH1C.1 | GOLIM4 | 0.70 | FG.Endth/Ven.ACKR1.MADCAM1 | LEPR | 1.17 | FG.Prolif/EC.UBE2C.TOP2A | SFN | 1.10 |
| FG.EC.DMBT1.ADH1C.1 | NDUFV1 | 0.69 | FG.Endth/Ven.ACKR1.MADCAM1 | PLK2 | 1.17 | FG.Prolif/EC.UBE2C.TOP2A | AKR1C3 | 1.09 |
| FG.EC.DMBT1.ADH1C.1 | SPINT2 | 0.69 | FG.Endth/Ven.ACKR1.MADCAM1 | MYCT1 | 1.17 | FG.Prolif/EC.UBE2C.TOP2A | IDH2 | 1.09 |
| FG.EC.DMBT1.ADH1C.1 | CISD1 | 0.69 | FG.Endth/Ven.ACKR1.MADCAM1 | NOSTRIN | 1.16 | FG.Prolif/EC.UBE2C.TOP2A | CPS1 | 1.08 |
| FG.EC.DMBT1.ADH1C.1 | KRT20 | 0.69 | FG.Endth/Ven.ACKR1.MADCAM1 | ACTN1 | 1.16 | FG.Prolif/EC.UBE2C.TOP2A | MGST2 | 1.04 |
| FG.EC.DMBT1.ADH1C.1 | HADHB | 0.69 | FG.Endth/Ven.ACKR1.MADCAM1 | ZNF385D | 1.15 | FG.Prolif/EC.UBE2C.TOP2A | CDKN3 | 1.04 |
| FG.EC.DMBT1.ADH1C.1 | ONECUT2 | 0.68 | FG.Endth/Ven.ACKR1.MADCAM1 | IL1R1 | 1.14 | FG.Prolif/EC.UBE2C.TOP2A | CDX1 | 1.04 |
| FG.EC.DMBT1.ADH1C.1 | RAB25 | 0.68 | FG.Endth/Ven.ACKR1.MADCAM1 | IFI44L | 1.14 | FG.Prolif/EC.UBE2C.TOP2A | CD9 | 1.04 |
| FG.EC.DMBT1.ADH1C.1 | KLF3 | 0.68 | FG.Endth/Ven.ACKR1.MADCAM1 | LMCD1 | 1.14 | FG.Prolif/EC.UBE2C.TOP2A | CDC20 | 1.04 |
| FG.EC.DMBT1.ADH1C.1 | MT1M | 0.68 | FG.Endth/Ven.ACKR1.MADCAM1 | TSPAN4 | 1.13 | FG.Prolif/EC.UBE2C.TOP2A | SMIM22 | 1.03 |
| FG.EC.DMBT1.ADH1C.1 | TMPRSS15 | 0.68 | FG.Endth/Ven.ACKR1.MADCAM1 | NRP1 | 1.13 | FG.Prolif/EC.UBE2C.TOP2A | BIRC5 | 1.03 |
| FG.EC.DMBT1.ADH1C.1 | ALDH2 | 0.67 | FG.Endth/Ven.ACKR1.MADCAM1 | ADGRL4 | 1.13 | FG.Prolif/EC.UBE2C.TOP2A | KIAA0101 | 1.03 |
| FG.EC.DMBT1.ADH1C.1 | RBM47 | 0.67 | FG.Endth/Ven.ACKR1.MADCAM1 | CAV2 | 1.13 | FG.Prolif/EC.UBE2C.TOP2A | PPP1R1B | 1.01 |
| FG.EC.DMBT1.ADH1C.1 | SLC4A7 | 0.66 | FG.Endth/Ven.ACKR1.MADCAM1 | ACVRL1 | 1.13 | FG.Prolif/EC.UBE2C.TOP2A | CDK1 | 1.01 |
| FG.EC.DMBT1.ADH1C.1 | BDH2 | 0.66 | FG.Endth/Ven.ACKR1.MADCAM1 | MMRN2 | 1.13 | FG.Prolif/EC.UBE2C.TOP2A | ASPM | 1.01 |
| FG.EC.DMBT1.ADH1C.1 | TST | 0.66 | FG.Endth/Ven.ACKR1.MADCAM1 | KANK3 | 1.12 | FG.Prolif/EC.UBE2C.TOP2A | RAMP1 | 1.00 |
| FG.EC.DMBT1.ADH1C.1 | TM9SF3 | 0.66 | FG.Endth/Ven.ACKR1.MADCAM1 | TMEM100 | 1.10 | FG.Prolif/EC.UBE2C.TOP2A | TMEM106C | 1.00 |
| FG.EC.DMBT1.ADH1C.1 | PCK2 | 0.66 | FG.Endth/Ven.ACKR1.MADCAM1 | CLIC2 | 1.09 | FG.Prolif/EC.UBE2C.TOP2A | UGT2B17 | 0.99 |
| FG.EC.DMBT1.ADH1C.1 | GCNT1 | 0.66 | FG.Endth/Ven.ACKR1.MADCAM1 | TINAGL1 | 1.09 | FG.Prolif/EC.UBE2C.TOP2A | CES2 | 0.99 |
| FG.EC.DMBT1.ADH1C.1 | SUCLG2 | 0.65 | FG.Endth/Ven.ACKR1.MADCAM1 | CYP1B1 | 1.06 | FG.Prolif/EC.UBE2C.TOP2A | CLDN15 | 0.97 |
| FG.EC.DMBT1.ADH1C.1 | AGPAT2 | 0.65 | FG.Endth/Ven.ACKR1.MADCAM1 | RASA4 | 1.05 | FG.Prolif/EC.UBE2C.TOP2A | CENPW | 0.97 |
| FG.EC.DMBT1.ADH1C.1 | DSG2 | 0.64 | FG.Endth/Ven.ACKR1.MADCAM1 | HSPG2 | 1.02 | FG.Prolif/EC.UBE2C.TOP2A | OAT | 0.96 |
| FG.EC.DMBT1.ADH1C.1 | SELENBP1 | 0.64 | FG.Endth/Ven.ACKR1.MADCAM1 | IL3RA | 1.01 | FG.Prolif/EC.UBE2C.TOP2A | ATP1B1 | 0.94 |
| FG.EC.DMBT1.ADH1C.1 | RP3-335E1.1 | 0.64 | FG.Endth/Ven.ACKR1.MADCAM1 | SPARC | 0.98 | FG.Prolif/EC.UBE2C.TOP2A | HMGCS2 | 0.93 |
| FG.EC.DMBT1.ADH1C.1 | NAPRT | 0.64 | FG.Endth/Ven.ACKR1.MADCAM1 | MKL2 | 0.96 | FG.Prolif/EC.UBE2C.TOP2A | CISD3 | 0.91 |
| FG.EC.DMBT1.ADH1C.1 | SLC2A2 | 0.63 | FG.Endth/Ven.ACKR1.MADCAM1 | TGFBR3 | 0.95 | FG.Prolif/EC.UBE2C.TOP2A | FABP1 | 0.91 |
| FG.EC.DMBT1.ADH1C.1 | GPX2 | 0.63 | FG.Endth/Ven.ACKR1.MADCAM1 | SMAD1 | 0.94 | FG.Prolif/EC.UBE2C.TOP2A | ALDH1A1 | 0.91 |
| FG.EC.DMBT1.ADH1C.1 | ECHS1 | 0.63 | FG.Endth/Ven.ACKR1.MADCAM1 | FLT1 | 0.94 | FG.Prolif/EC.UBE2C.TOP2A | FAM3B | 0.89 |
| FG.EC.DMBT1.ADH1C.1 | TXNDC17 | 0.63 | FG.Endth/Ven.ACKR1.MADCAM1 | GIMAP6 | 0.94 | FG.Prolif/EC.UBE2C.TOP2A | CKS1B | 0.89 |
| FG.EC.DMBT1.ADH1C.1 | STAP2 | 0.63 | FG.Endth/Ven.ACKR1.MADCAM1 | NRN1 | 0.93 | FG.Prolif/EC.UBE2C.TOP2A | CCNA2 | 0.89 |
| FG.EC.DMBT1.ADH1C.1 | ADH4 | 0.63 | FG.Endth/Ven.ACKR1.MADCAM1 | SELP | 0.91 | FG.Prolif/EC.UBE2C.TOP2A | PTGR1 | 0.88 |
| FG.EC.DMBT1.ADH1C.1 | PRR15L | 0.62 | FG.Endth/Ven.ACKR1.MADCAM1 | PKP4 | 0.90 | FG.Prolif/EC.UBE2C.TOP2A | RRM2 | 0.88 |
| FG.EC.DMBT1.ADH1C.1 | GLS | 0.62 | FG.Endth/Ven.ACKR1.MADCAM1 | NUAK1 | 0.90 | FG.Prolif/EC.UBE2C.TOP2A | SMC4 | 0.87 |
| FG.EC.DMBT1.ADH1C.1 | CYBRD1 | 0.62 | FG.Endth/Ven.ACKR1.MADCAM1 | CXorf36 | 0.89 | FG.Prolif/EC.UBE2C.TOP2A | EDN1 | 0.87 |
| FG.EC.DMBT1.ADH1C.1 | CDH1 | 0.62 | FG.Endth/Ven.ACKR1.MADCAM1 | MMRN1 | 0.89 | FG.Prolif/EC.UBE2C.TOP2A | DTYMK | 0.87 |
| FG.EC.DMBT1.ADH1C.1 | ADD3 | 0.62 | FG.Endth/Ven.ACKR1.MADCAM1 | SLCO2A1 | 0.89 | FG.Prolif/EC.UBE2C.TOP2A | CCNB2 | 0.86 |
| FG.EC.DMBT1.ADH1C.1 | GPR160 | 0.61 | FG.Endth/Ven.ACKR1.MADCAM1 | OLFM1 | 0.85 | FG.Prolif/EC.UBE2C.TOP2A | HADH | 0.86 |
| FG.EC.DMBT1.ADH1C.1 | HNF4A | 0.61 | FG.Endth/Ven.ACKR1.MADCAM1 | TEK | 0.84 | FG.Prolif/EC.UBE2C.TOP2A | HMMR | 0.85 |
| FG.EC.DMBT1.ADH1C.1 | PERP | 0.61 | FG.Endth/Ven.ACKR1.MADCAM1 | RASIP1 | 0.84 | FG.Prolif/EC.UBE2C.TOP2A | DHRS11 | 0.83 |
| FG.EC.DMBT1.ADH1C.1 | TMEM41A | 0.60 | FG.Endth/Ven.ACKR1.MADCAM1 | FILIP1 | 0.83 | FG.Prolif/EC.UBE2C.TOP2A | HMGB3 | 0.83 |
| FG.EC.DMBT1.ADH1C.1 | DECR1 | 0.60 | FG.Endth/Ven.ACKR1.MADCAM1 | RAB3C | 0.83 | FG.Prolif/EC.UBE2C.TOP2A | GPR160 | 0.82 |
| FG.EC.DMBT1.ADH1C.1 | FOLH1 | 0.60 | FG.Endth/Ven.ACKR1.MADCAM1 | ERG | 0.82 | FG.Prolif/EC.UBE2C.TOP2A | AURKB | 0.82 |
| FG.EC.DMBT1.ADH1C.1 | SDHB | 0.60 | FG.Endth/Ven.ACKR1.MADCAM1 | PCDH17 | 0.81 | FG.Prolif/EC.UBE2C.TOP2A | MAOA | 0.81 |
| FG.EC.DMBT1.ADH1C.1 | USH1C | 0.60 | FG.Endth/Ven.ACKR1.MADCAM1 | RAI14 | 0.81 | FG.Prolif/EC.UBE2C.TOP2A | LIMA1 | 0.81 |
| FG.EC.DMBT1.ADH1C.1 | TPM1 | 0.60 | FG.Endth/Ven.ACKR1.MADCAM1 | FAM110D | 0.81 | FG.Prolif/EC.UBE2C.TOP2A | TMEM54 | 0.79 |
| FG.EC.DMBT1.ADH1C.1 | BLVRB | 0.59 | FG.Endth/Ven.ACKR1.MADCAM1 | BCAT1 | 0.80 | FG.Prolif/EC.UBE2C.TOP2A | KRTCAP3 | 0.79 |
| FG.EC.DMBT1.ADH1C.1 | SLC22A18 | 0.59 | FG.Endth/Ven.ACKR1.MADCAM1 | LDB2 | 0.80 | FG.Prolif/EC.UBE2C.TOP2A | PBK | 0.78 |
| FG.EC.DMBT1.ADH1C.1 | NDUFS3 | 0.59 | FG.Endth/Ven.ACKR1.MADCAM1 | PALMD | 0.80 | FG.Prolif/EC.UBE2C.TOP2A | S100A14 | 0.77 |
| FG.EC.DMBT1.ADH1C.1 | OLFM4 | 0.59 | FG.Endth/Ven.ACKR1.MADCAM1 | TPD52L1 | 0.80 | FG.Prolif/EC.UBE2C.TOP2A | S100A16 | 0.77 |
| FG.EC.DMBT1.ADH1C.1 | MS4A8 | 0.59 | FG.Endth/Ven.ACKR1.MADCAM1 | LCN6 | 0.76 | FG.Prolif/EC.UBE2C.TOP2A | ETHE1 | 0.76 |
| FG.EC.DMBT1.ADH1C.1 | GNA11 | 0.59 | FG.Endth/Ven.ACKR1.MADCAM1 | FAM155A | 0.76 | FG.Prolif/EC.UBE2C.TOP2A | PLP2 | 0.75 |
| FG.EC.DMBT1.ADH1C.1 | CDHR5 | 0.59 | FG.Endth/Ven.ACKR1.MADCAM1 | FBLN2 | 0.74 | FG.Prolif/EC.UBE2C.TOP2A | HIST1H1D | 0.75 |
| FG.EC.DMBT1.ADH1C.1 | DGAT1 | 0.59 | FG.Endth/Ven.ACKR1.MADCAM1 | MEOX1 | 0.74 | FG.Prolif/EC.UBE2C.TOP2A | PLA2G2A | 0.75 |
| FG.EC.DMBT1.ADH1C.1 | ETFA | 0.58 | FG.Endth/Ven.ACKR1.MADCAM1 | ZNF521 | 0.74 | FG.Prolif/EC.UBE2C.TOP2A | LAD1 | 0.74 |
| FG.EC.DMBT1.ADH1C.1 | MYH14 | 0.58 | FG.Endth/Ven.ACKR1.MADCAM1 | CDH5 | 0.74 | FG.Prolif/EC.UBE2C.TOP2A | CHP2 | 0.74 |
| FG.EC.DMBT1.ADH1C.1 | CCND1 | 0.57 | FG.Endth/Ven.ACKR1.MADCAM1 | TIE1 | 0.73 | FG.Prolif/EC.UBE2C.TOP2A | DSP | 0.74 |
| FG.EC.DMBT1.ADH1C.1 | NDUFA10 | 0.57 | FG.Endth/Ven.ACKR1.MADCAM1 | NTN4 | 0.72 | FG.Prolif/EC.UBE2C.TOP2A | STRA13 | 0.74 |
| FG.EC.DMBT1.ADH1C.1 | TSC22D1 | 0.57 | FG.Endth/Ven.ACKR1.MADCAM1 | PGM5 | 0.71 | FG.Prolif/EC.UBE2C.TOP2A | TK1 | 0.74 |
| FG.EC.DMBT1.ADH1C.1 | CAT | 0.57 | FG.Endth/Ven.ACKR1.MADCAM1 | PTPRM | 0.71 | FG.Prolif/EC.UBE2C.TOP2A | MAD2L1 | 0.73 |
| FG.EC.DMBT1.ADH1C.1 | SULT1B1 | 0.57 | FG.Endth/Ven.ACKR1.MADCAM1 | CLEC3B | 0.71 | FG.Prolif/EC.UBE2C.TOP2A | SMC2 | 0.73 |
| FG.EC.DMBT1.ADH1C.1 | PLPP2 | 0.57 | FG.Endth/Ven.ACKR1.MADCAM1 | TLL1 | 0.70 | FG.Prolif/EC.UBE2C.TOP2A | EBP | 0.73 |
| FG.EC.DMBT1.ADH1C.1 | FAM3D | 0.57 | FG.Endth/Ven.ACKR1.MADCAM1 | SPHK1 | 0.70 | FG.Prolif/EC.UBE2C.TOP2A | ANXA4 | 0.72 |
| FG.EC.DMBT1.ADH1C.1 | ERBB3 | 0.57 | FG.Endth/Ven.ACKR1.MADCAM1 | ROBO4 | 0.67 | FG.Prolif/EC.UBE2C.TOP2A | PRC1 | 0.72 |
| FG.EC.DMBT1.ADH1C.1 | SLC39A14 | 0.56 | FG.Endth/Ven.ACKR1.MADCAM1 | CNTNAP3B | 0.63 | FG.Prolif/EC.UBE2C.TOP2A | UBE2T | 0.72 |
| FG.EC.DMBT1.ADH1C.1 | MTCH2 | 0.56 | FG.Endth/Ven.ACKR1.MADCAM1 | LHX6 | 0.60 | FG.Prolif/EC.UBE2C.TOP2A | ZWINT | 0.72 |
| FG.EC.DMBT1.ADH1C.1 | ETS2 | 0.56 | FG.Endth/Ven.ACKR1.MADCAM1 | MYRIP | 0.58 | FG.Prolif/EC.UBE2C.TOP2A | CDX2 | 0.70 |
| FG.EC.DMBT1.ADH1C.1 | IDH1 | 0.56 | FG.Endth/Ven.ACKR1.MADCAM1 | CD109 | 0.58 | FG.Prolif/EC.UBE2C.TOP2A | GOLM1 | 0.70 |
| FG.EC.DMBT1.ADH1C.1 | AC004556.1 | 0.56 | FG.Endth/Ven.ACKR1.MADCAM1 | ADCY4 | 0.57 | FG.Prolif/EC.UBE2C.TOP2A | CMBL | 0.69 |
| FG.EC.DMBT1.ADH1C.1 | SLC39A4 | 0.56 | FG.Endth/Ven.ACKR1.MADCAM1 | DOC2B | 0.56 | FG.Prolif/EC.UBE2C.TOP2A | HNF4A | 0.69 |
| FG.EC.DMBT1.ADH1C.1 | PRSS8 | 0.56 | FG.Endth/Ven.ACKR1.MADCAM1 | DUSP23 | 1.55 | FG.Prolif/EC.UBE2C.TOP2A | TPX2 | 0.69 |
| FG.EC.DMBT1.ADH1C.1 | TCEA3 | 0.56 | FG.Endth/Ven.ACKR1.MADCAM1 | VWA1 | 0.81 | FG.Prolif/EC.UBE2C.TOP2A | AQP1 | 0.69 |
| FG.EC.DMBT1.ADH1C.1 | CFTR | 0.56 | FG.Endth/Ven.ACKR1.MADCAM1 | ADGRL2 | 0.71 | FG.Prolif/EC.UBE2C.TOP2A | DSG2 | 0.69 |
| FG.EC.DMBT1.ADH1C.1 | GIPC2 | 0.55 | FG.Endth/Ven.ACKR1.MADCAM1 | THBD | 0.85 | FG.Prolif/EC.UBE2C.TOP2A | AURKA | 0.68 |
| FG.EC.DMBT1.ADH1C.1 | MGAT4B | 0.55 | FG.Endth/Ven.ACKR1.MADCAM1 | ITM2A | 1.46 | FG.Prolif/EC.UBE2C.TOP2A | EMP2 | 0.68 |
| FG.EC.DMBT1.ADH1C.1 | EBP | 0.55 | FG.Enteroendocrine.SCG3.ISL1 | CHGA | 4.85 | FG.Prolif/EC.UBE2C.TOP2A | OTC | 0.68 |
| FG.EC.DMBT1.ADH1C.1 | MYO1A | 0.55 | FG.Enteroendocrine.SCG3.ISL1 | PCSK1N | 3.97 | FG.Prolif/EC.UBE2C.TOP2A | CENPA | 0.68 |
| FG.EC.DMBT1.ADH1C.1 | ZNF593 | 0.54 | FG.Enteroendocrine.SCG3.ISL1 | PYY | 3.79 | FG.Prolif/EC.UBE2C.TOP2A | PXMP2 | 0.67 |
| FG.EC.DMBT1.ADH1C.1 | MUC13 | 0.54 | FG.Enteroendocrine.SCG3.ISL1 | CHGB | 2.71 | FG.Prolif/EC.UBE2C.TOP2A | PERP | 0.67 |
| FG.EC.DMBT1.ADH1C.1 | B3GNT5 | 0.53 | FG.Enteroendocrine.SCG3.ISL1 | TPH1 | 2.57 | FG.Prolif/EC.UBE2C.TOP2A | AHCY | 0.66 |
| FG.EC.DMBT1.ADH1C.1 | EPHX2 | 0.53 | FG.Enteroendocrine.SCG3.ISL1 | GC | 2.52 | FG.Prolif/EC.UBE2C.TOP2A | CDH17 | 0.66 |
| FG.EC.DMBT1.ADH1C.1 | ATP8B1 | 0.53 | FG.Enteroendocrine.SCG3.ISL1 | SCG2 | 2.34 | FG.Prolif/EC.UBE2C.TOP2A | TYMS | 0.65 |
| FG.EC.DMBT1.ADH1C.1 | GNG12 | 0.53 | FG.Enteroendocrine.SCG3.ISL1 | SCG5 | 2.30 | FG.Prolif/EC.UBE2C.TOP2A | CNN3 | 0.65 |
| FG.EC.DMBT1.ADH1C.1 | DEGS2 | 0.53 | FG.Enteroendocrine.SCG3.ISL1 | CES1 | 2.17 | FG.Prolif/EC.UBE2C.TOP2A | CDCA3 | 0.64 |
| FG.EC.DMBT1.ADH1C.1 | ACTN4 | 0.53 | FG.Enteroendocrine.SCG3.ISL1 | FEV | 2.06 | FG.Prolif/EC.UBE2C.TOP2A | RAB25 | 0.64 |
| FG.EC.DMBT1.ADH1C.1 | CCDC47 | 0.53 | FG.Enteroendocrine.SCG3.ISL1 | TTR | 2.06 | FG.Prolif/EC.UBE2C.TOP2A | GGH | 0.64 |
| FG.EC.DMBT1.ADH1C.1 | CRLS1 | 0.52 | FG.Enteroendocrine.SCG3.ISL1 | CPE | 1.98 | FG.Prolif/EC.UBE2C.TOP2A | TMEM98 | 0.63 |
| FG.EC.DMBT1.ADH1C.1 | CDX2 | 0.52 | FG.Enteroendocrine.SCG3.ISL1 | SCGN | 1.97 | FG.Prolif/EC.UBE2C.TOP2A | TMPO | 0.62 |
| FG.EC.DMBT1.ADH1C.1 | CBLC | 0.52 | FG.Enteroendocrine.SCG3.ISL1 | CRYBA2 | 1.92 | FG.Prolif/EC.UBE2C.TOP2A | VIL1 | 0.62 |
| FG.EC.DMBT1.ADH1C.1 | SLC35A3 | 0.52 | FG.Enteroendocrine.SCG3.ISL1 | NEUROD1 | 1.87 | FG.Prolif/EC.UBE2C.TOP2A | CCDC34 | 0.62 |
| FG.EC.DMBT1.ADH1C.1 | PTPRF | 0.52 | FG.Enteroendocrine.SCG3.ISL1 | ARC | 1.63 | FG.Prolif/EC.UBE2C.TOP2A | PLK1 | 0.61 |
| FG.EC.DMBT1.ADH1C.1 | TMEM54 | 0.51 | FG.Enteroendocrine.SCG3.ISL1 | PCSK1 | 1.50 | FG.Prolif/EC.UBE2C.TOP2A | HMGN5 | 0.61 |
| FG.EC.DMBT1.ADH1C.1 | SLC3A1 | 0.51 | FG.Enteroendocrine.SCG3.ISL1 | HEPACAM2 | 1.42 | FG.Prolif/EC.UBE2C.TOP2A | C2orf82 | 0.60 |
| FG.EC.DMBT1.ADH1C.1 | PXMP2 | 0.51 | FG.Enteroendocrine.SCG3.ISL1 | SSTR2 | 1.33 | FG.Prolif/EC.UBE2C.TOP2A | GGCT | 0.60 |
| FG.EC.DMBT1.ADH1C.1 | COMT | 0.51 | FG.Enteroendocrine.SCG3.ISL1 | ADGRG4 | 1.30 | FG.Prolif/EC.UBE2C.TOP2A | DLGAP5 | 0.59 |
| FG.EC.DMBT1.ADH1C.1 | CDS1 | 0.51 | FG.Enteroendocrine.SCG3.ISL1 | ISL1 | 1.21 | FG.Prolif/EC.UBE2C.TOP2A | GPSM2 | 0.59 |
| FG.EC.DMBT1.ADH1C.1 | HADH | 0.51 | FG.Enteroendocrine.SCG3.ISL1 | PSCA | 1.16 | FG.Prolif/EC.UBE2C.TOP2A | GTSE1 | 0.58 |
| FG.EC.DMBT1.ADH1C.1 | FXYD3 | 0.51 | FG.Enteroendocrine.SCG3.ISL1 | CADPS | 1.12 | FG.Prolif/EC.UBE2C.TOP2A | GSTM4 | 0.58 |
| FG.EC.DMBT1.ADH1C.1 | AP1M2 | 0.51 | FG.Enteroendocrine.SCG3.ISL1 | VTN | 1.12 | FG.Prolif/EC.UBE2C.TOP2A | PGRMC1 | 0.58 |
| FG.EC.DMBT1.ADH1C.1 | ADA | 0.51 | FG.Enteroendocrine.SCG3.ISL1 | MIR7-3HG | 1.09 | FG.Prolif/EC.UBE2C.TOP2A | RAD51AP1 | 0.58 |
| FG.EC.DMBT1.ADH1C.1 | RNF186 | 0.50 | FG.Enteroendocrine.SCG3.ISL1 | NKX2-2 | 1.04 | FG.Prolif/EC.UBE2C.TOP2A | SPC25 | 0.57 |
| FG.EC.DMBT1.ADH1C.1 | VNN1 | 0.50 | FG.Enteroendocrine.SCG3.ISL1 | BEX1 | 1.01 | FG.Prolif/EC.UBE2C.TOP2A | H2AFX | 0.57 |
| FG.EC.DMBT1.ADH1C.1 | AVPI1 | 0.50 | FG.Enteroendocrine.SCG3.ISL1 | RAB26 | 0.99 | FG.Prolif/EC.UBE2C.TOP2A | PKP2 | 0.57 |
| FG.EC.DMBT1.ADH1C.1 | CREB3L3 | 0.50 | FG.Enteroendocrine.SCG3.ISL1 | SCG3 | 0.97 | FG.Prolif/EC.UBE2C.TOP2A | FABP2 | 0.57 |
| FG.EC.DMBT1.ADH1C.1 | NDRG1 | 0.50 | FG.Enteroendocrine.SCG3.ISL1 | SMIM6 | 0.95 | FG.Prolif/EC.UBE2C.TOP2A | KIF20B | 0.56 |
| FG.EC.DMBT1.ADH1C.1 | TOB1 | 0.50 | FG.Enteroendocrine.SCG3.ISL1 | INSM1 | 0.94 | FG.Prolif/EC.UBE2C.TOP2A | CENPE | 0.56 |
| FG.EC.DMBT1.ADH1C.1 | ACADS | 0.50 | FG.Enteroendocrine.SCG3.ISL1 | CACNA2D1 | 0.88 | FG.Prolif/EC.UBE2C.TOP2A | CENPN | 0.56 |
| FG.EC.DMBT1.ADH1C.1 | MVP | 0.50 | FG.Enteroendocrine.SCG3.ISL1 | VWA5B2 | 0.85 | FG.Prolif/EC.UBE2C.TOP2A | RNASE1 | 0.55 |
| FG.EC.DMBT1.ADH1C.1 | AOC1 | 0.50 | FG.Enteroendocrine.SCG3.ISL1 | SNAP25 | 0.85 | FG.Prolif/EC.UBE2C.TOP2A | FAM84A | 0.55 |
| FG.EC.DMBT1.ADH1C.1 | TUBB2A | 0.49 | FG.Enteroendocrine.SCG3.ISL1 | RAB3B | 0.85 | FG.Prolif/EC.UBE2C.TOP2A | ESRP1 | 0.55 |
| FG.EC.DMBT1.ADH1C.1 | APP | 0.49 | FG.Enteroendocrine.SCG3.ISL1 | RPRML | 0.80 | FG.Prolif/EC.UBE2C.TOP2A | MXD3 | 0.55 |
| FG.EC.DMBT1.ADH1C.1 | TMPRSS4 | 0.49 | FG.Enteroendocrine.SCG3.ISL1 | PAX6 | 0.75 | FG.Prolif/EC.UBE2C.TOP2A | KIAA1324 | 0.55 |
| FG.EC.DMBT1.ADH1C.1 | IQGAP2 | 0.49 | FG.Enteroendocrine.SCG3.ISL1 | CACNA1A | 0.73 | FG.Prolif/EC.UBE2C.TOP2A | PLCB3 | 0.54 |
| FG.EC.DMBT1.ADH1C.1 | ABHD11 | 0.49 | FG.Enteroendocrine.SCG3.ISL1 | CALY | 0.71 | FG.Prolif/EC.UBE2C.TOP2A | ECT2 | 0.54 |
| FG.EC.DMBT1.ADH1C.1 | CDC42BPA | 0.49 | FG.Enteroendocrine.SCG3.ISL1 | SLC29A4 | 0.71 | FG.Prolif/EC.UBE2C.TOP2A | TCEA3 | 0.54 |
| FG.EC.DMBT1.ADH1C.1 | SLC37A4 | 0.49 | FG.Enteroendocrine.SCG3.ISL1 | RGS4 | 0.70 | FG.Prolif/EC.UBE2C.TOP2A | CDH1 | 0.53 |
| FG.EC.DMBT1.ADH1C.1 | MYO6 | 0.49 | FG.Enteroendocrine.SCG3.ISL1 | RIMBP2 | 0.69 | FG.Prolif/EC.UBE2C.TOP2A | ACADS | 0.53 |
| FG.EC.DMBT1.ADH1C.1 | CASP1 | 0.49 | FG.Enteroendocrine.SCG3.ISL1 | ST18 | 0.69 | FG.Prolif/EC.UBE2C.TOP2A | NDC80 | 0.53 |
| FG.EC.DMBT1.ADH1C.1 | SLC13A2 | 0.49 | FG.Enteroendocrine.SCG3.ISL1 | UCN3 | 0.59 | FG.Prolif/EC.UBE2C.TOP2A | LMNB1 | 0.53 |
| FG.EC.DMBT1.ADH1C.1 | AP1S1 | 0.49 | FG.Enteroendocrine.SCG3.ISL1 | KCNJ6 | 0.57 | FG.Prolif/EC.UBE2C.TOP2A | GJB1 | 0.53 |
| FG.EC.DMBT1.ADH1C.1 | ASL | 0.49 | FG.Enteroendocrine.SCG3.ISL1 | C2orf54 | 0.57 | FG.Prolif/EC.UBE2C.TOP2A | MAL2 | 0.53 |
| FG.EC.DMBT1.ADH1C.1 | TMC5 | 0.49 | FG.Enteroendocrine.SCG3.ISL1 | KCNK16 | 0.51 | FG.Prolif/EC.UBE2C.TOP2A | GOT1 | 0.52 |
| FG.EC.DMBT1.ADH1C.1 | SLC4A4 | 0.48 | FG.Enteroendocrine.SCG3.ISL1 | ONECUT3 | 0.46 | FG.Prolif/EC.UBE2C.TOP2A | SGOL2 | 0.52 |
| FG.EC.DMBT1.ADH1C.1 | AK3 | 0.48 | FG.Enteroendocrine.SCG3.ISL1 | KCNK3 | 0.45 | FG.Prolif/EC.UBE2C.TOP2A | APP | 0.52 |
| FG.EC.DMBT1.ADH1C.1 | GMDS | 0.48 | FG.Enteroendocrine.SCG3.ISL1 | ARX | 0.45 | FG.Prolif/EC.UBE2C.TOP2A | SLC39A5 | 0.51 |
| FG.EC.DMBT1.ADH1C.1 | EPS8L3 | 0.48 | FG.Enteroendocrine.SCG3.ISL1 | CELF3 | 0.44 | FG.Prolif/EC.UBE2C.TOP2A | STAP2 | 0.51 |
| FG.EC.DMBT1.ADH1C.1 | MRPS12 | 0.48 | FG.Enteroendocrine.SCG3.ISL1 | PTPRN | 0.43 | FG.Prolif/EC.UBE2C.TOP2A | DDAH1 | 0.51 |
| FG.EC.DMBT1.ADH1C.1 | ACSL5 | 0.48 | FG.Enteroendocrine.SCG3.ISL1 | KCNH6 | 0.42 | FG.Prolif/EC.UBE2C.TOP2A | SRPK1 | 0.51 |
| FG.EC.DMBT1.ADH1C.1 | HDHD3 | 0.47 | FG.Enteroendocrine.SCG3.ISL1 | TAGLN3 | 0.42 | FG.Prolif/EC.UBE2C.TOP2A | HDGF | 0.51 |
| FG.EC.DMBT1.ADH1C.1 | PLCB3 | 0.47 | FG.Enteroendocrine.SCG3.ISL1 | RFX6 | 0.40 | FG.Prolif/EC.UBE2C.TOP2A | KIFC1 | 0.51 |
| FG.EC.DMBT1.ADH1C.1 | MAL2 | 0.47 | FG.Enteroendocrine.SCG3.ISL1 | AC068535.3 | 0.40 | FG.Prolif/EC.UBE2C.TOP2A | PRSS8 | 0.51 |
| FG.EC.DMBT1.ADH1C.1 | ITGA6 | 0.47 | FG.Enteroendocrine.SCG3.ISL1 | ABCC8 | 0.39 | FG.Prolif/EC.UBE2C.TOP2A | CENPV | 0.51 |
| FG.EC.DMBT1.ADH1C.1 | SERINC2 | 0.47 | FG.Enteroendocrine.SCG3.ISL1 | KIF1A | 0.36 | FG.Prolif/EC.UBE2C.TOP2A | TST | 0.50 |
| FG.EC.DMBT1.ADH1C.1 | LAD1 | 0.47 | FG.Enteroendocrine.SCG3.ISL1 | ST8SIA3 | 0.35 | FG.Prolif/EC.UBE2C.TOP2A | MYO6 | 0.50 |
| FG.EC.DMBT1.ADH1C.1 | CLRN3 | 0.46 | FG.Enteroendocrine.SCG3.ISL1 | LINC00907 | 0.35 | FG.Prolif/EC.UBE2C.TOP2A | DEPDC1 | 0.50 |
| FG.EC.DMBT1.ADH1C.1 | ESRRA | 0.46 | FG.Enteroendocrine.SCG3.ISL1 | ETV1 | 0.46 | FG.Prolif/EC.UBE2C.TOP2A | HIST1H1B | 0.50 |
| FG.EC.DMBT1.ADH1C.1 | SLC20A1 | 0.46 | FG.EpithStem.LGR5.SMOC2 | OLFM4 | 3.43 | FG.Prolif/EC.UBE2C.TOP2A | IGFBP2 | 0.50 |
| FG.EC.DMBT1.ADH1C.1 | MIR22HG | 0.46 | FG.EpithStem.LGR5.SMOC2 | REG1A | 3.18 | FG.Prolif/EC.UBE2C.TOP2A | EPHB2 | 0.50 |
| FG.EC.DMBT1.ADH1C.1 | AHCYL1 | 0.46 | FG.EpithStem.LGR5.SMOC2 | SPINK1 | 2.71 | FG.Prolif/EC.UBE2C.TOP2A | IFT172 | 0.50 |
| FG.EC.DMBT1.ADH1C.1 | FUOM | 0.46 | FG.EpithStem.LGR5.SMOC2 | TSPAN8 | 2.64 | FG.Prolif/EC.UBE2C.TOP2A | EHF | 0.50 |
| FG.EC.DMBT1.ADH1C.1 | LDLR | 0.46 | FG.EpithStem.LGR5.SMOC2 | KRT18 | 2.23 | FG.Prolif/EC.UBE2C.TOP2A | PRR15L | 0.49 |
| FG.EC.DMBT1.ADH1C.1 | PPP1R14D | 0.46 | FG.EpithStem.LGR5.SMOC2 | DEFA5 | 2.11 | FG.Prolif/EC.UBE2C.TOP2A | STARD10 | 0.49 |
| FG.EC.DMBT1.ADH1C.1 | TMEM98 | 0.45 | FG.EpithStem.LGR5.SMOC2 | DEFA6 | 2.09 | FG.Prolif/EC.UBE2C.TOP2A | PLEK2 | 0.49 |
| FG.EC.DMBT1.ADH1C.1 | MRPS35 | 0.45 | FG.EpithStem.LGR5.SMOC2 | SLC12A2 | 1.92 | FG.Prolif/EC.UBE2C.TOP2A | KIF11 | 0.49 |
| FG.EC.DMBT1.ADH1C.1 | MRPS25 | 0.45 | FG.EpithStem.LGR5.SMOC2 | KRT8 | 1.84 | FG.Prolif/EC.UBE2C.TOP2A | ARHGAP11A | 0.48 |
| FG.EC.DMBT1.ADH1C.1 | GGT6 | 0.45 | FG.EpithStem.LGR5.SMOC2 | GPX2 | 1.82 | FG.Prolif/EC.UBE2C.TOP2A | NUF2 | 0.48 |
| FG.EC.DMBT1.ADH1C.1 | CENPV | 0.45 | FG.EpithStem.LGR5.SMOC2 | REG3A | 1.78 | FG.Prolif/EC.UBE2C.TOP2A | CERS6 | 0.48 |
| FG.EC.DMBT1.ADH1C.1 | FUT2 | 0.45 | FG.EpithStem.LGR5.SMOC2 | PLA2G2A | 1.78 | FG.Prolif/EC.UBE2C.TOP2A | IMPA2 | 0.48 |
| FG.EC.DMBT1.ADH1C.1 | PDE4C | 0.45 | FG.EpithStem.LGR5.SMOC2 | EDN1 | 1.74 | FG.Prolif/EC.UBE2C.TOP2A | AP1M2 | 0.48 |
| FG.EC.DMBT1.ADH1C.1 | IFT172 | 0.45 | FG.EpithStem.LGR5.SMOC2 | EPCAM | 1.74 | FG.Prolif/EC.UBE2C.TOP2A | ATP8B1 | 0.48 |
| FG.EC.DMBT1.ADH1C.1 | FAM213A | 0.45 | FG.EpithStem.LGR5.SMOC2 | CLDN4 | 1.72 | FG.Prolif/EC.UBE2C.TOP2A | AKAP1 | 0.47 |
| FG.EC.DMBT1.ADH1C.1 | DDAH1 | 0.45 | FG.EpithStem.LGR5.SMOC2 | CLDN3 | 1.70 | FG.Prolif/EC.UBE2C.TOP2A | NEK2 | 0.47 |
| FG.EC.DMBT1.ADH1C.1 | SPINT1 | 0.45 | FG.EpithStem.LGR5.SMOC2 | AGR2 | 1.70 | FG.Prolif/EC.UBE2C.TOP2A | FERMT1 | 0.47 |
| FG.EC.DMBT1.ADH1C.1 | ACOT7 | 0.45 | FG.EpithStem.LGR5.SMOC2 | MT1E | 1.54 | FG.Prolif/EC.UBE2C.TOP2A | KCNJ13 | 0.47 |
| FG.EC.DMBT1.ADH1C.1 | FAM162A | 0.44 | FG.EpithStem.LGR5.SMOC2 | LGALS4 | 1.46 | FG.Prolif/EC.UBE2C.TOP2A | TMEM45B | 0.47 |
| FG.EC.DMBT1.ADH1C.1 | DHRS4L2 | 0.44 | FG.EpithStem.LGR5.SMOC2 | MT1G | 1.44 | FG.Prolif/EC.UBE2C.TOP2A | PLPP2 | 0.46 |
| FG.EC.DMBT1.ADH1C.1 | ZG16 | 0.44 | FG.EpithStem.LGR5.SMOC2 | ASCL2 | 1.43 | FG.Prolif/EC.UBE2C.TOP2A | AC004556.1 | 0.46 |
| FG.EC.DMBT1.ADH1C.1 | PLEK2 | 0.44 | FG.EpithStem.LGR5.SMOC2 | GDF15 | 1.41 | FG.Prolif/EC.UBE2C.TOP2A | CENPM | 0.46 |
| FG.EC.DMBT1.ADH1C.1 | ST14 | 0.44 | FG.EpithStem.LGR5.SMOC2 | KLF5 | 1.38 | FG.Prolif/EC.UBE2C.TOP2A | FXYD3 | 0.46 |
| FG.EC.DMBT1.ADH1C.1 | CLINT1 | 0.44 | FG.EpithStem.LGR5.SMOC2 | MT1F | 1.35 | FG.Prolif/EC.UBE2C.TOP2A | CEP55 | 0.46 |
| FG.EC.DMBT1.ADH1C.1 | PLS1 | 0.44 | FG.EpithStem.LGR5.SMOC2 | SOX4 | 1.35 | FG.Prolif/EC.UBE2C.TOP2A | CENPK | 0.46 |
| FG.EC.DMBT1.ADH1C.1 | EPN1 | 0.44 | FG.EpithStem.LGR5.SMOC2 | ATP1B1 | 1.32 | FG.Prolif/EC.UBE2C.TOP2A | DHFR | 0.45 |
| FG.EC.DMBT1.ADH1C.1 | GALM | 0.44 | FG.EpithStem.LGR5.SMOC2 | ELF3 | 1.28 | FG.Prolif/EC.UBE2C.TOP2A | NCAPG | 0.45 |
| FG.EC.DMBT1.ADH1C.1 | SLC34A2 | 0.44 | FG.EpithStem.LGR5.SMOC2 | PPP1R1B | 1.28 | FG.Prolif/EC.UBE2C.TOP2A | HSD11B2 | 0.45 |
| FG.EC.DMBT1.ADH1C.1 | CAMK2N1 | 0.44 | FG.EpithStem.LGR5.SMOC2 | TFF3 | 1.27 | FG.Prolif/EC.UBE2C.TOP2A | ESCO2 | 0.45 |
| FG.EC.DMBT1.ADH1C.1 | PDHA1 | 0.44 | FG.EpithStem.LGR5.SMOC2 | HMGCS2 | 1.26 | FG.Prolif/EC.UBE2C.TOP2A | CCL15 | 0.45 |
| FG.EC.DMBT1.ADH1C.1 | A1CF | 0.43 | FG.EpithStem.LGR5.SMOC2 | UGT2B17 | 1.22 | FG.Prolif/EC.UBE2C.TOP2A | SGOL1 | 0.45 |
| FG.EC.DMBT1.ADH1C.1 | RP11-532F12.5 | 0.43 | FG.EpithStem.LGR5.SMOC2 | PRSS2 | 1.18 | FG.Prolif/EC.UBE2C.TOP2A | ALDH7A1 | 0.45 |
| FG.EC.DMBT1.ADH1C.1 | TMC4 | 0.43 | FG.EpithStem.LGR5.SMOC2 | ETS2 | 1.15 | FG.Prolif/EC.UBE2C.TOP2A | CFTR | 0.44 |
| FG.EC.DMBT1.ADH1C.1 | QTRT1 | 0.43 | FG.EpithStem.LGR5.SMOC2 | CLDN7 | 1.14 | FG.Prolif/EC.UBE2C.TOP2A | CDC42EP5 | 0.44 |
| FG.EC.DMBT1.ADH1C.1 | TMEM30B | 0.43 | FG.EpithStem.LGR5.SMOC2 | AGR3 | 1.14 | FG.Prolif/EC.UBE2C.TOP2A | GIPC2 | 0.44 |
| FG.EC.DMBT1.ADH1C.1 | TM4SF20 | 0.43 | FG.EpithStem.LGR5.SMOC2 | RAMP1 | 1.11 | FG.Prolif/EC.UBE2C.TOP2A | ATP2C2 | 0.44 |
| FG.EC.DMBT1.ADH1C.1 | BCL2L15 | 0.43 | FG.EpithStem.LGR5.SMOC2 | GPR160 | 1.10 | FG.Prolif/EC.UBE2C.TOP2A | KIF23 | 0.44 |
| FG.EC.DMBT1.ADH1C.1 | CD68 | 0.43 | FG.EpithStem.LGR5.SMOC2 | RGMB | 1.08 | FG.Prolif/EC.UBE2C.TOP2A | CASP6 | 0.43 |
| FG.EC.DMBT1.ADH1C.1 | DSC2 | 0.42 | FG.EpithStem.LGR5.SMOC2 | SMIM22 | 1.07 | FG.Prolif/EC.UBE2C.TOP2A | CDCA8 | 0.43 |
| FG.EC.DMBT1.ADH1C.1 | TSPAN3 | 0.42 | FG.EpithStem.LGR5.SMOC2 | CD9 | 1.06 | FG.Prolif/EC.UBE2C.TOP2A | MGAT4B | 0.43 |
| FG.EC.DMBT1.ADH1C.1 | GPD1 | 0.42 | FG.EpithStem.LGR5.SMOC2 | MGST1 | 1.02 | FG.Prolif/EC.UBE2C.TOP2A | CASC5 | 0.43 |
| FG.EC.DMBT1.ADH1C.1 | KCNE3 | 0.42 | FG.EpithStem.LGR5.SMOC2 | QTRT1 | 1.00 | FG.Prolif/EC.UBE2C.TOP2A | GSTO2 | 0.43 |
| FG.EC.DMBT1.ADH1C.1 | LMO7 | 0.42 | FG.EpithStem.LGR5.SMOC2 | SMOC2 | 1.00 | FG.Prolif/EC.UBE2C.TOP2A | ERBB3 | 0.43 |
| FG.EC.DMBT1.ADH1C.1 | SLC9A3R1 | 0.42 | FG.EpithStem.LGR5.SMOC2 | ALDH1B1 | 0.99 | FG.Prolif/EC.UBE2C.TOP2A | ABHD11 | 0.43 |
| FG.EC.DMBT1.ADH1C.1 | PVRL3 | 0.42 | FG.EpithStem.LGR5.SMOC2 | CDX1 | 0.97 | FG.Prolif/EC.UBE2C.TOP2A | DDC | 0.42 |
| FG.EC.DMBT1.ADH1C.1 | TOM1L1 | 0.42 | FG.EpithStem.LGR5.SMOC2 | FAM3B | 0.96 | FG.Prolif/EC.UBE2C.TOP2A | SOX9 | 0.42 |
| FG.EC.DMBT1.ADH1C.1 | SLC51B | 0.41 | FG.EpithStem.LGR5.SMOC2 | CDX2 | 0.90 | FG.Prolif/EC.UBE2C.TOP2A | HPGD | 0.42 |
| FG.EC.DMBT1.ADH1C.1 | SCTR | 0.41 | FG.EpithStem.LGR5.SMOC2 | SELENBP1 | 0.87 | FG.Prolif/EC.UBE2C.TOP2A | NFIB | 0.42 |
| FG.EC.DMBT1.ADH1C.1 | CYP27A1 | 0.41 | FG.EpithStem.LGR5.SMOC2 | CDK6 | 0.85 | FG.Prolif/EC.UBE2C.TOP2A | MLXIP | 0.42 |
| FG.EC.DMBT1.ADH1C.1 | CD2AP | 0.41 | FG.EpithStem.LGR5.SMOC2 | PERP | 0.84 | FG.Prolif/EC.UBE2C.TOP2A | HJURP | 0.41 |
| FG.EC.DMBT1.ADH1C.1 | EFHD2 | 0.41 | FG.EpithStem.LGR5.SMOC2 | STARD10 | 0.84 | FG.Prolif/EC.UBE2C.TOP2A | SYCE1L | 0.41 |
| FG.EC.DMBT1.ADH1C.1 | PCSK5 | 0.41 | FG.EpithStem.LGR5.SMOC2 | MAOA | 0.83 | FG.Prolif/EC.UBE2C.TOP2A | PTPRF | 0.41 |
| FG.EC.DMBT1.ADH1C.1 | BRI3 | 0.41 | FG.EpithStem.LGR5.SMOC2 | PDZK1IP1 | 0.82 | FG.Prolif/EC.UBE2C.TOP2A | CKAP5 | 0.41 |
| FG.EC.DMBT1.ADH1C.1 | CYP2C18 | 0.41 | FG.EpithStem.LGR5.SMOC2 | CDCA7 | 0.82 | FG.Prolif/EC.UBE2C.TOP2A | MACROD1 | 0.40 |
| FG.EC.DMBT1.ADH1C.1 | CTNND1 | 0.41 | FG.EpithStem.LGR5.SMOC2 | RNF43 | 0.81 | FG.Prolif/EC.UBE2C.TOP2A | KIF15 | 0.40 |
| FG.EC.DMBT1.ADH1C.1 | ECI1 | 0.41 | FG.EpithStem.LGR5.SMOC2 | DPEP1 | 0.79 | FG.Prolif/EC.UBE2C.TOP2A | CKMT1B | 0.40 |
| FG.EC.DMBT1.ADH1C.1 | GLYCTK | 0.40 | FG.EpithStem.LGR5.SMOC2 | ITGA6 | 0.79 | FG.Prolif/EC.UBE2C.TOP2A | TMEM97 | 0.40 |
| FG.EC.DMBT1.ADH1C.1 | CAPNS1 | 0.40 | FG.EpithStem.LGR5.SMOC2 | LGR5 | 0.77 | FG.Prolif/EC.UBE2C.TOP2A | ALDH1B1 | 0.40 |
| FG.EC.DMBT1.ADH1C.1 | LGALS2 | 0.40 | FG.EpithStem.LGR5.SMOC2 | ADH1C | 0.77 | FG.Prolif/EC.UBE2C.TOP2A | EPS8 | 0.40 |
| FG.EC.DMBT1.ADH1C.1 | CPT1A | 0.40 | FG.EpithStem.LGR5.SMOC2 | TMEM98 | 0.76 | FG.Prolif/EC.UBE2C.TOP2A | RNASEH2A | 0.39 |
| FG.EC.DMBT1.ADH1C.1 | AKAP1 | 0.40 | FG.EpithStem.LGR5.SMOC2 | CNN3 | 0.76 | FG.Prolif/EC.UBE2C.TOP2A | C9orf152 | 0.39 |
| FG.EC.DMBT1.ADH1C.1 | PLGRKT | 0.40 | FG.EpithStem.LGR5.SMOC2 | RAB25 | 0.75 | FG.Prolif/EC.UBE2C.TOP2A | CKMT1A | 0.39 |
| FG.EC.DMBT1.ADH1C.1 | CDC42EP5 | 0.40 | FG.EpithStem.LGR5.SMOC2 | KLK1 | 0.72 | FG.Prolif/EC.UBE2C.TOPZA | RACGAP1 | 0.39 |
| FG.EC.DMBT1.ADH1C.1 | OVOL1 | 0.40 | FG.EpithStem.LGR5.SMOC2 | KRTCAP3 | 0.72 | FG.Prolif/EC.UBE2C.TOP2A | CTBP2 | 0.39 |
| FG.EC.DMBT1.ADH1C.1 | HSD17B4 | 0.40 | FG.EpithStem.LGR5.SMOC2 | KIAA1324 | 0.70 | FG.Prolif/EC.UBE2C.TOP2A | FAM83D | 0.38 |
| FG.EC.DMBT1.ADH1C.1 | PKP2 | 0.39 | FG.EpithStem.LGR5.SMOC2 | AKR1C3 | 0.69 | FG.Prolif/EC.UBE2C.TOP2A | LRRCC1 | 0.38 |
| FG.EC.DMBT1.ADH1C.1 | EPS8 | 0.39 | FG.EpithStem.LGR5.SMOC2 | FAM84A | 0.67 | FG.Prolif/EC.UBE2C.TOP2A | MELK | 0.38 |
| FG.EC.DMBT1.ADH1C.1 | MIR194-2HG | 0.39 | FG.EpithStem.LGR5.SMOC2 | SOX9 | 0.64 | FG.Prolif/EC.UBE2C.TOP2A | TMPRSS4 | 0.38 |
| FG.EC.DMBT1.ADH1C.1 | EHF | 0.39 | FG.EpithStem.LGR5.SMOC2 | ALDH1A1 | 0.63 | FG.Prolif/EC.UBE2C.TOP2A | STXBP6 | 0.38 |
| FG.EC.DMBT1.ADH1C.1 | PKP3 | 0.39 | FG.EpithStem.LGR5.SMOC2 | ITLN2 | 0.62 | FG.Prolif/EC.UBE2C.TOP2A | TMEM171 | 0.38 |
| FG.EC.DMBT1.ADH1C.1 | CLDN23 | 0.39 | FG.EpithStem.LGR5.SMOC2 | RP11-357H14.17 | 0.62 | FG.Prolif/EC.UBE2C.TOP2A | FAM3D | 0.38 |
| FG.EC.DMBT1.ADH1C.1 | TLCD2 | 0.39 | FG.EpithStem.LGR5.SMOC2 | CERS6 | 0.62 | FG.Prolif/EC.UBE2C.TOP2A | ASF1B | 0.37 |
| FG.EC.DMBT1.ADH1C.1 | CKMT1B | 0.39 | FG.EpithStem.LGR5.SMOC2 | AQP1 | 0.61 | FG.Prolif/EC.UBE2C.TOP2A | GGT6 | 0.37 |
| FG.EC.DMBT1.ADH1C.1 | PGRMC1 | 0.39 | FG.EpithStem.LGR5.SMOC2 | IFT172 | 0.61 | FG.Prolif/EC.UBE2C.TOP2A | PARPBP | 0.36 |
| FG.EC.DMBT1.ADH1C.1 | NFIB | 0.38 | FG.EpithStem.LGR5.SMOC2 | ZNF703 | 0.60 | FG.Prolif/EC.UBE2C.TOP2A | ANLN | 0.36 |
| FG.EC.DMBT1.ADH1C.1 | CD46 | 0.38 | FG.EpithStem.LGR5.SMOC2 | STXBP6 | 0.59 | FG.Prolif/EC.UBE2C.TOP2A | WHSC1 | 0.35 |
| FG.EC.DMBT1.ADH1C.1 | LLGL2 | 0.38 | FG.EpithStem.LGR5.SMOC2 | EPHB2 | 0.58 | FG.Prolif/EC.UBE2C.TOP2A | TROAP | 0.35 |
| FG.EC.DMBT1.ADH1C.1 | ACO1 | 0.38 | FG.EpithStem.LGR5.SMOC2 | TRABD2A | 0.57 | FG.Prolif/EC.UBE2C.TOP2A | TTK | 0.35 |
| FG.EC.DMBT1.ADH1C.1 | C17orf78 | 0.38 | FG.EpithStem.LGR5.SMOC2 | CXCL3 | 0.57 | FG.Prolif/EC.UBE2C.TOP2A | FUT3 | 0.35 |
| FG.EC.DMBT1.ADH1C.1 | SLC44A4 | 0.38 | FG.EpithStem.LGR5.SMOC2 | CXADR | 0.56 | FG.Prolif/EC.UBE2C.TOP2A | KIF4A | 0.35 |
| FG.EC.DMBT1.ADH1C.1 | AIFM1 | 0.38 | FG.EpithStem.LGR5.SMOC2 | TCEA3 | 0.54 | FG.Prolif/EC.UBE2C.TOP2A | FOXM1 | 0.35 |
| FG.EC.DMBT1.ADH1C.1 | NDUFS1 | 0.38 | FG.EpithStem.LGR5.SMOC2 | URAD | 0.53 | FG.Prolif/EC.UBE2C.TOP2A | MIS18A | 0.35 |
| FG.EC.DMBT1.ADH1C.1 | AIMP2 | 0.38 | FG.EpithStem.LGR5.SMOC2 | OSR2 | 0.53 | FG.Prolif/EC.UBE2C.TOP2A | NCAPD2 | 0.35 |
| FG.EC.DMBT1.ADH1C.1 | ALDH3A2 | 0.38 | FG.EpithStem.LGR5.SMOC2 | FERMT1 | 0.53 | FG.Prolif/EC.UBE2C.TOP2A | KIF2C | 0.34 |
| FG.EC.DMBT1.ADH1C.1 | SHROOM3 | 0.38 | FG.EpithStem.LGR5.SMOC2 | FXYD3 | 0.52 | FG.Prolif/EC.UBE2C.TOP2A | PRTFDC1 | 0.34 |
| FG.EC.DMBT1.ADH1C.1 | WASL | 0.38 | FG.EpithStem.LGR5.SMOC2 | KCNJ13 | 0.51 | FG.Prolif/EC.UBE2C.TOP2A | CDCA2 | 0.34 |
| FG.EC.DMBT1.ADH1C.1 | GATA4 | 0.37 | FG.EpithStem.LGR5.SMOC2 | GPRC5A | 0.51 | FG.Prolif/EC.UBE2C.TOP2A | F12 | 0.34 |
| FG.EC.DMBT1.ADH1C.1 | ACOX1 | 0.37 | FG.EpithStem.LGR5.SMOC2 | EPHB3 | 0.50 | FG.Prolif/EC.UBE2C.TOP2A | BUB1 | 0.34 |
| FG.EC.DMBT1.ADH1C.1 | VDR | 0.37 | FG.EpithStem.LGR5.SMOC2 | HOXA9 | 0.50 | FG.Prolif/EC.UBE2C.TOP2A | ZNF703 | 0.33 |
| FG.EC.DMBT1.ADH1C.1 | MSRB2 | 0.37 | FG.EpithStem.LGR5.SMOC2 | PLCB4 | 0.50 | FG.Prolif/EC.UBE2C.TOP2A | SAPCD2 | 0.33 |
| FG.EC.DMBT1.ADH1C.1 | SLC28A2 | 0.37 | FG.EpithStem.LGR5.SMOC2 | CYR61 | 0.50 | FG.Prolif/EC.UBE2C.TOP2A | DIAPH3 | 0.33 |
| FG.EC.DMBT1.ADH1C.1 | ABCC3 | 0.37 | FG.EpithStem.LGR5.SMOC2 | CA12 | 0.49 | FG.Prolif/EC.UBE2C.TOP2A | DEPDC1B | 0.33 |
| FG.EC.DMBT1.ADH1C.1 | SCRN2 | 0.37 | FG.EpithStem.LGR5.SMOC2 | TSPAN6 | 0.49 | FG.Prolif/EC.UBE2C.TOP2A | MYO10 | 0.33 |
| FG.EC.DMBT1.ADH1C.1 | FAHD1 | 0.37 | FG.EpithStem.LGR5.SMOC2 | C9orf152 | 0.46 | FG.Prolif/EC.UBE2C.TOP2A | PRELID2 | 0.32 |
| FG.EC.DMBT1.ADH1C.1 | HSDL2 | 0.37 | FG.EpithStem.LGR5.SMOC2 | VSIG10 | 0.45 | FG.Prolif/EC.UBE2C.TOP2A | OIP5 | 0.32 |
| FG.EC.DMBT1.ADH1C.1 | GDA | 0.37 | FG.EpithStem.LGR5.SMOC2 | C2orf82 | 0.43 | FG.Prolif/EC.UBE2C.TOP2A | PRR11 | 0.32 |
| FG.EC.DMBT1.ADH1C.1 | SLC35G1 | 0.36 | FG.EpithStem.LGR5.SMOC2 | SNHG18 | 0.42 | FG.Prolif/EC.UBE2C.TOP2A | SPDL1 | 0.32 |
| FG.EC.DMBT1.ADH1C.1 | RETSAT | 0.36 | FG.EpithStem.LGR5.SMOC2 | FUT3 | 0.41 | FG.Prolif/EC.UBE2C.TOP2A | RHOBTB3 | 0.31 |
| FG.EC.DMBT1.ADH1C.1 | SLC7A9 | 0.36 | FG.EpithStem.LGR5.SMOC2 | CDHR1 | 0.41 | FG.Prolif/EC.UBE2C.TOP2A | FAAH | 0.31 |
| FG.EC.DMBT1.ADH1C.1 | MARVELD3 | 0.36 | FG.EpithStem.LGR5.SMOC2 | PKDCC | 0.41 | FG.Prolif/EC.UBE2C.TOP2A | ST6GALNAC1 | 0.31 |
| FG.EC.DMBT1.ADH1C.1 | ACOT11 | 0.36 | FG.EpithStem.LGR5.SMOC2 | SLC44A3 | 0.40 | FG.Prolif/EC.UBE2C.TOP2A | CA9 | 0.31 |
| FG.EC.DMBT1.ADH1C.1 | ANKRD9 | 0.36 | FG.EpithStem.LGR5.SMOC2 | C10orf99 | 0.39 | FG.Prolif/EC.UBE2C.TOP2A | TRIM2 | 0.30 |
| FG.EC.DMBT1.ADH1C.1 | AC004754.3 | 0.36 | FG.EpithStem.LGR5.SMOC2 | CHDH | 0.39 | FG.Prolif/EC.UBE2C.TOP2A | KIF14 | 0.30 |
| FG.EC.DMBT1.ADH1C.1 | LUZP1 | 0.36 | FG.EpithStem.LGR5.SMOC2 | FOXA3 | 0.39 | FG.Prolif/EC.UBE2C.TOP2A | COQ2 | 0.30 |
| FG.EC.DMBT1.ADH1C.1 | TNFRSF12A | 0.36 | FG.EpithStem.LGR5.SMOC2 | ID4 | 0.38 | FG.Prolif/EC.UBE2C.TOP2A | CDC25C | 0.29 |
| FG.EC.DMBT1.ADH1C.1 | C6orf132 | 0.36 | FG.EpithStem.LGR5.SMOC2 | ATP2C2 | 0.36 | FG.Prolif/EC.UBE2C.TOP2A | LINC01224 | 0.29 |
| FG.EC.DMBT1.ADH1C.1 | C2orf82 | 0.36 | FG.EpithStem.LGR5.SMOC2 | CLDN2 | 0.33 | FG.Prolif/EC.UBE2C.TOP2A | IQGAP3 | 0.28 |
| FG.EC.DMBT1.ADH1C.1 | GJB1 | 0.35 | FG.EpithStem.LGR5.SMOC2 | HOXB7 | 0.33 | FG.Prolif/EC.UBE2C.TOP2A | SLC25A10 | 0.28 |
| FG.EC.DMBT1.ADH1C.1 | NQO1 | 0.35 | FG.EpithStem.LGR5.SMOC2 | PTPRO | 0.33 | FG.Prolif/EC.UBE2C.TOP2A | NCAPH | 0.27 |
| FG.EC.DMBT1.ADH1C.1 | LRPPRC | 0.35 | FG.EpithStem.LGR5.SMOC2 | MYB | 0.32 | FG.Prolif/EC.UBE2C.TOP2A | CCNF | 0.27 |
| FG.EC.DMBT1.ADH1C.1 | TM6SF2 | 0.35 | FG.EpithStem.LGR5.SMOC2 | HOXD1 | 0.31 | FG.Prolif/EC.UBE2C.TOP2A | CDCA5 | 0.26 |
| FG.EC.DMBT1.ADH1C.1 | CNN3 | 0.35 | FG.EpithStem.LGR5.SMOC2 | UGT2B15 | 0.29 | FG.Prolif/EC.UBE2C.TOP2A | KCNQ1 | 0.26 |
| FG.EC.DMBT1.ADH1C.1 | CXADR | 0.35 | FG.EpithStem.LGR5.SMOC2 | PITX2 | 0.28 | FG.Prolif/EC.UBE2C.TOP2A | SKA3 | 0.26 |
| FG.EC.DMBT1.ADH1C.1 | GALNT3 | 0.35 | FG.EpithStem.LGR5.SMOC2 | TRIM2 | 0.42 | FG.Prolif/EC.UBE2C.TOP2A | LGR4 | 0.25 |
| FG.EC.DMBT1.ADH1C.1 | C1orf21 | 0.35 | FG.EpithStem.LGR5.SMOC2 | PHGR1 | 0.94 | FG.Prolif/EC.UBE2C.TOP2A | GCNT1 | 0.51 |
| FG.EC.DMBT1.ADH1C.1 | GCNT3 | 0.35 | FG.EpithStem.LGR5.SMOC2 | ARSE | 0.35 | FG.Prolif/EC.UBE2C.TOP2A | URAD | 0.39 |
| FG.EC.DMBT1.ADH1C.1 | SULT1C2 | 0.35 | FG.EpithStem.LGR5.SMOC2 | TINAGL1 | 0.41 | FG.Prolif/EC.UBE2C.TOP2A | GMDS | 0.57 |
| FG.EC.DMBT1.ADH1C.1 | PBX1 | 0.35 | FG.EpithStem.LGR5.SMOC2 | DSG2 | 0.55 | FG.Prolif/EC.UBE2C.TOP2A | PDZK1IP1 | 0.27 |
| FG.EC.DMBT1.ADH1C.1 | TMEM171 | 0.34 | FG.EpithStem.LGR5.SMOC2 | PGAP1 | 0.37 | FG.Prolif/EC.UBE2C.TOP2A | GATA6 | 0.28 |
| FG.EC.DMBT1.ADH1C.1 | GALE | 0.34 | FG.EpithStem.LGR5.SMOC2 | PIGR | 0.94 | FG.Prolif/EC.UBE2C.TOP2A | ABO | 0.34 |
| FG.EC.DMBT1.ADH1C.1 | ACE2 | 0.34 | FG.EpithStem.LGR5.SMOC2 | MLXIP | 0.56 | FG.Prolif/EC.UBE2C.TOP2A | HACD3 | 0.46 |
| FG.EC.DMBT1.ADH1C.1 | MEP1A | 0.34 | FG.EpithStem.LGR5.SMOC2 | RHOB | 0.94 | FG.Prolif/EC.UBE2C.TOP2A | C14orf80 | 0.29 |
| FG.EC.DMBT1.ADH1C.1 | KIAA1324 | 0.34 | FG.EpithStem.SMOC2.RGMB | SMOC2 | 1.90 | FG.Prolif/EC.UBE2C.TOP2A | C10orf99 | 0.25 |
| FG.EC.DMBT1.ADH1C.1 | CALML4 | 0.34 | FG.EpithStem.SMOC2.RGMB | PMFBP1 | 0.27 | FG.Prolif/EC.UBE2C.TOP2A | RRM1 | 0.40 |
| FG.EC.DMBT1.ADH1C.1 | IRF6 | 0.34 | FG.Fibro.ACKR3.RCN3 | APOE | 3.25 | FG.Prolif/EC.UBE2C.TOP2A | ZDHHC12 | 0.55 |
| FG.EC.DMBT1.ADH1C.1 | TJP3 | 0.34 | FG.Fibro.ACKR3.RCN3 | CCL11 | 3.23 | FG.Prolif/EC.UBE2C.TOP2A | GFPT1 | 0.40 |
| FG.EC.DMBT1.ADH1C.1 | SULT1E1 | 0.34 | FG.Fibro.ACKR3.RCN3 | CFD | 3.18 | FG.Prolif/EC.UBE2C.TOP2A | RP11-357H14.17 | 0.35 |
| FG.EC.DMBT1.ADH1C.1 | RP11-680F8.1 | 0.34 | FG.Fibro.ACKR3.RCN3 | CCL2 | 3.04 | FG.Prolif/EC.UBE2C.TOP2A | EPS8L3 | 0.44 |
| FG.EC.DMBT1.ADH1C.1 | FAT1 | 0.33 | FG.Fibro.ACKR3.RCN3 | MFAP4 | 3.00 | FG.Prolif/EC.UBE2C.TOP2A | BORA | 0.35 |
| FG.EC.DMBT1.ADH1C.1 | CKMT1A | 0.33 | FG.Fibro.ACKR3.RCN3 | LUM | 2.97 | FG.Prolif/EC.UBE2C.TOP2A | GALNT3 | 0.33 |
| FG.EC.DMBT1.ADH1C.1 | ARHGDIG | 0.33 | FG.Fibro.ACKR3.RCN3 | CXCL14 | 2.95 | FG.Prolif/EC.UBE2C.TOP2A | MRPL12 | 0.78 |
| FG.EC.DMBT1.ADH1C.1 | CERS6 | 0.33 | FG.Fibro.ACKR3.RCN3 | DCN | 2.77 | FG.Prolif/EC.UBE2C.TOP2A | SHMT1 | 0.38 |
| FG.EC.DMBT1.ADH1C.1 | HNF4G | 0.33 | FG.Fibro.ACKR3.RCN3 | C1S | 2.64 | FG.Prolif/EC.UBE2C.TOP2A | PVRL3 | 0.27 |
| FG.EC.DMBT1.ADH1C.1 | SLC12A2 | 0.33 | FG.Fibro.ACKR3.RCN3 | C1R | 2.57 | FG.Prolif/EC.UBE2C.TOP2A | SUCLG2 | 0.75 |
| FG.EC.DMBT1.ADH1C.1 | MAOB | 0.33 | FG.Fibro.ACKR3.RCN3 | RARRES2 | 2.55 | FG.Prolif/EC.UBE2C.TOP2A | LGALS2 | 0.93 |
| FG.EC.DMBT1.ADH1C.1 | GUCY2C | 0.33 | FG.Fibro.ACKR3.RCN3 | FBLN1 | 2.35 | FG.Prolif/EC.UBE2C.TOP2A | ETS2 | 0.72 |
| FG.EC.DMBT1.ADH1C.1 | LINC01207 | 0.33 | FG.Fibro.ACKR3.RCN3 | CCL8 | 2.33 | FG.Prolif/EC.UBE2C.TOP2A | ITGA6 | 0.52 |
| FG.EC.DMBT1.ADH1C.1 | NUDT8 | 0.33 | FG.Fibro.ACKR3.RCN3 | CALD1 | 2.32 | FG.Prolif/EC.UBE2C.TOP2A | ACAA2 | 0.67 |
| FG.EC.DMBT1.ADH1C.1 | ABO | 0.33 | FG.Fibro.ACKR3.RCN3 | CXCL12 | 2.26 | FG.Prolif/EC.UBE2C.TOP2A | LBR | 0.62 |
| FG.EC.DMBT1.ADH1C.1 | NIPSNAP1 | 0.32 | FG.Fibro.ACKR3.RCN3 | COL1A2 | 2.22 | FG.Prolif/EC.UBE2C.TOP2A | SERINC2 | 0.42 |
| FG.EC.DMBT1.ADH1C.1 | TMPRSS2 | 0.32 | FG.Fibro.ACKR3.RCN3 | PMP22 | 2.20 | FG.T.CCL5.CD8B | CCL5 | 2.36 |
| FG.EC.DMBT1.ADH1C.1 | FZD5 | 0.32 | FG.Fibro.ACKR3.RCN3 | CFH | 2.12 | FG.T.CCL5.CD8B | CD8B | 1.51 |
| FG.EC.DMBT1.ADH1C.1 | TMEM253 | 0.32 | FG.Fibro.ACKR3.RCN3 | PPP1R14A | 2.11 | FG.T.CCL5.CD8B | CD8A | 1.47 |
| FG.EC.DMBT1.ADH1C.1 | MYO10 | 0.31 | FG.Fibro.ACKR3.RCN3 | QSOX1 | 2.09 | FG.T.CCL5.CD8B | IL7R | 1.43 |
| FG.EC.DMBT1.ADH1C.1 | NBL1 | 0.31 | FG.Fibro.ACKR3.RCN3 | CXCL1 | 2.06 | FG.T.CCL5.CD8B | TNFAIP3 | 1.37 |
| FG.EC.DMBT1.ADH1C.1 | TRIM54 | 0.31 | FG.Fibro.ACKR3.RCN3 | RBP1 | 2.06 | FG.T.CCL5.CD8B | AC092580.4 | 1.21 |
| FG.EC.DMBT1.ADH1C.1 | GOLT1A | 0.31 | FG.Fibro.ACKR3.RCN3 | PTN | 2.02 | FG.T.GZMK.CMC1 | GZMK | 2.54 |
| FG.EC.DMBT1.ADH1C.1 | C11orf54 | 0.31 | FG.Fibro.ACKR3.RCN3 | GGT5 | 2.02 | FG.T.GZMK.CRTAM | GZMK | 2.73 |
| FG.EC.DMBT1.ADH1C.1 | FERMT1 | 0.31 | FG.Fibro.ACKR3.RCN3 | TM4SF1 | 1.97 | FG.T.GZMK.CRTAM | CCL5 | 2.35 |
| FG.EC.DMBT1.ADH1C.1 | MAP7 | 0.31 | FG.Fibro.ACKR3.RCN3 | CYGB | 1.97 | FG.T.GZMK.CRTAM | NKG7 | 2.16 |
| FG.EC.DMBT1.ADH1C.1 | SPDEF | 0.30 | FG.Fibro.ACKR3.RCN3 | COL3A1 | 1.96 | FG.T.GZMK.CRTAM | CST7 | 2.15 |
| FG.EC.DMBT1.ADH1C.1 | NR1H4 | 0.30 | FG.Fibro.ACKR3.RCN3 | HAPLN1 | 1.95 | FG.T.GZMK.CRTAM | GZMA | 2.02 |
| FG.EC.DMBT1.ADH1C.1 | ADTRP | 0.30 | FG.Fibro.ACKR3.RCN3 | PLTP | 1.93 | FG.T.GZMK.CRTAM | CCL4 | 1.84 |
| FG.EC.DMBT1.ADH1C.1 | FNBP1L | 0.30 | FG.Fibro.ACKR3.RCN3 | EMILIN1 | 1.92 | FG.T.GZMK.GZMA | GZMK | 2.72 |
| FG.EC.DMBT1.ADH1C.1 | ATRN | 0.30 | FG.Fibro.ACKR3.RCN3 | GPX3 | 1.91 | FG.T.GZMK.GZMA | GZMA | 2.17 |
| FG.EC.DMBT1.ADH1C.1 | RAPGEFL1 | 0.30 | FG.Fibro.ACKR3.RCN3 | MMP2 | 1.87 | FG.T.GZMK.IFNG | GZMK | 2.69 |
| FG.EC.DMBT1.ADH1C.1 | CORO2A | 0.29 | FG.Fibro.ACKR3.RCN3 | COL6A2 | 1.82 | FG.T.GZMK.NKG7 | GZMK | 2.54 |
| FG.EC.DMBT1.ADH1C.1 | C9orf152 | 0.29 | FG.Fibro.ACKR3.RCN3 | MEG3 | 1.82 | FG.T.GZMK.NKG7 | GZMA | 2.36 |
| FG.EC.DMBT1.ADH1C.1 | TRIM2 | 0.29 | FG.Fibro.ACKR3.RCN3 | TCF21 | 1.81 | FG.T.MT-ND4L.TSC22D3 | ANXA1 | 1.89 |
| FG.EC.DMBT1.ADH1C.1 | HOOK1 | 0.29 | FG.Fibro.ACKR3.RCN3 | PLPP3 | 1.81 | FG.T.MT-ND4L.TSC22D3 | TSC22D3 | 1.81 |
| FG.EC.DMBT1.ADH1C.1 | GPA33 | 0.29 | FG.Fibro.ACKR3.RCN3 | SPARC | 1.77 | FG.T.MT-ND4L.TSC22D3 | IL7R | 1.72 |
| FG.EC.DMBT1.ADH1C.1 | LINC00675 | 0.28 | FG.Fibro.ACKR3.RCN3 | SERPING1 | 1.74 | FG.T.MT-ND4L.TSC22D3 | CCL5 | 1.70 |
| FG.EC.DMBT1.ADH1C.1 | TPMT | 0.28 | FG.Fibro.ACKR3.RCN3 | FHL1 | 1.73 | FG.T.MT-ND4L.TSC22D3 | CD96 | 1.66 |
| FG.EC.DMBT1.ADH1C.1 | XDH | 0.28 | FG.Fibro.ACKR3.RCN3 | PLAC9 | 1.68 | FG.T.MT-ND4L.TSC22D3 | CD3D | 1.42 |
| FG.EC.DMBT1.ADH1C.1 | PFKFB2 | 0.28 | FG.Fibro.ACKR3.RCN3 | ABCA8 | 1.63 | FG.T.MT-ND4L.TSC22D3 | B2M | 1.28 |
| FG.EC.DMBT1.ADH1C.1 | GSTO2 | 0.28 | FG.Fibro.ACKR3.RCN3 | PCOLCE | 1.58 | FG.T.MT-ND4L.TSC22D3 | TMSB4X | 1.03 |
| FG.EC.DMBT1.ADH1C.1 | BCL2L14 | 0.28 | FG.Fibro.ACKR3.RCN3 | EDIL3 | 1.57 | FG.T.MT-ND4L.TSC22D3 | MALAT1 | 0.64 |
| FG.EC.DMBT1.ADH1C.1 | FUT4 | 0.28 | FG.Fibro.ACKR3.RCN3 | SPON2 | 1.55 | FG.T.RORA.CCR6 | KLRB1 | 1.95 |
| FG.EC.DMBT1.ADH1C.1 | GK5 | 0.27 | FG.Fibro.ACKR3.RCN3 | VCAN | 1.51 | FG.T.RORA.CCR6 | CCL20 | 1.68 |
| FG.EC.DMBT1.ADH1C.1 | AXDND1 | 0.27 | FG.Fibro.ACKR3.RCN3 | VCAM1 | 1.46 | FG.T.RORA.CCR6 | TNFAIP3 | 1.64 |
| FG.EC.DMBT1.ADH1C.1 | C1orf106 | 0.27 | FG.Fibro.ACKR3.RCN3 | COL1A1 | 1.42 | FG.T.RORA.CCR6 | IL7R | 1.60 |
| FG.EC.DMBT1.ADH1C.1 | ACKR4 | 0.27 | FG.Fibro.ACKR3.RCN3 | ANGPTL1 | 1.38 | FG.T.RORA.CCR6 | S100A4 | 1.49 |
| FG.EC.DMBT1.ADH1C.1 | ADH6 | 0.27 | FG.Fibro.ACKR3.RCN3 | CLEC14A | 1.37 | FG.T.RORA.CCR6 | ANXA1 | 1.38 |
| FG.EC.DMBT1.ADH1C.1 | ARHGAP32 | 0.26 | FG.Fibro.ACKR3.RCN3 | SNAI2 | 1.36 | FG.T.RORA.CCR6 | RORA | 1.34 |
| FG.EC.DMBT1.ADH1C.1 | GRTP1 | 0.26 | FG.Fibro.ACKR3.RCN3 | NKX2-3 | 1.32 | FG.T.RORA.CCR6 | CD3D | 1.18 |
| FG.EC.DMBT1.ADH1C.1 | AFG3L2 | 0.33 | FG.Fibro.ACKR3.RCN3 | LOXL1 | 1.27 | FG.T.RORA.CCR6 | AC092580.4 | 1.15 |
| FG.EC.DMBT1.ADH1C.1 | AKR7A2 | 0.49 | FG.Fibro.ACKR3.RCN3 | BMP5 | 1.21 | FG.T.RORA.CCR6 | CD2 | 1.14 |
| FG.EC.DMBT1.ADH1C.1 | CCDC25 | 0.41 | FG.Fibro.ACKR3.RCN3 | BMP4 | 1.20 | FG.T.RORA.CCR6 | TRAC | 1.09 |
| FG.EC.DMBT1.ADH1C.1 | HADHA | 0.76 | FG.Fibro.ACKR3.RCN3 | CRISPLD2 | 1.10 | FG.T.RORA.CCR6 | AC020571.3 | 1.05 |
| FG.EC.DMBT1.ADH1C.1 | BAIAP2L2 | 0.35 | FG.Fibro.ACKR3.RCN3 | KCNS3 | 1.10 | FG.T.RORA.CCR6 | CD3E | 0.95 |
| FG.EC.DMBT1.ADH1C.1 | PDK4 | 0.38 | FG.Fibro.ACKR3.RCN3 | PCDH7 | 1.01 | FG.T.RORA.CCR6 | RPLP1 | 0.75 |
| FG.EC.DMBT1.ADH1C.1 | CEP70 | 0.29 | FG.Fibro.ACKR3.RCN3 | SCARA5 | 0.95 | FG.T.RORA.CCR6 | TMSB4X | 0.61 |
| FG.EC.DMBT1.ADH1C.1 | AK2 | 0.47 | FG.Fibro.ACKR3.RCN3 | ACKR3 | 0.95 | FG.T.RORA.CCR6 | B2M | 0.58 |
| FG.EC.DMBT1.ADH1C.1 | HSD11B2 | 0.30 | FG.Fibro.ACKR3.RCN3 | PROCR | 1.35 | FG.T.RORA.CCR6 | CD69 | 1.08 |
| FG.EC.DMBT1.ADH1C.1 | EPHX1 | 0.34 | FG.Fibro.ACKR3.RCN3 | NUPR1 | 1.51 | FG.T.RORA.CCR6 | ICOS | 0.97 |
| FG.EC.DMBT1.ADH1C.1 | PTPRK | 0.27 | FG.Fibro.ACTA2.SOSTDC1 | ACTA2 | 3.43 | FG.T.RORA.CCR6 | IL32 | 0.88 |
| FG.EC.DMBT1.ADH1C.1 | NDUFA8 | 0.45 | FG.Fibro.ACTA2.SOSTDC1 | TAGLN | 2.64 | FG.T.TRDC.SOX4 | TRDC | 2.22 |
| FG.EC.DMBT1.ADH1C.1 | MEIS2 | 0.31 | FG.Fibro.ACTA2.SOSTDC1 | WFDC1 | 2.19 | FG.T.TRDC.SOX4 | TRGC2 | 1.62 |
| FG.EC.DMBT1.ADH1C.1 | ECI2 | 0.37 | FG.Fibro.ACTA2.SOSTDC1 | MYLK | 2.11 | FG.T.TRDC.SOX4 | CCL5 | 1.60 |
| FG.EC.DMBT1.ADH1C.1 | TUBA1C | 0.44 | FG.Fibro.ACTA2.SOSTDC1 | HHIP | 1.14 | FG.T.TRDC.ZNF683 | TRDC | 2.72 |
| FG.EC.DMBT1.ADH1C.1 | F12 | 0.29 | FG.Fibro.ACTA2.TAGLN | ACTA2 | 3.40 | FG.T.TRDC.ZNF683 | CD7 | 1.90 |
| FG.EC.DMBT1.ADH1C.1 | JUP | 0.32 | FG.Fibro.ACTA2.TAGLN | TAGLN | 2.66 | FG.T.TRDC.ZNF683 | CCL5 | 1.81 |
| FG.EC.DMBT1.ADH1C.1 | CTBP2 | 0.29 | FG.Fibro.ACTA2.TAGLN | WFDC1 | 1.87 | FG.T.TRDC.ZNF683 | TRGC2 | 1.74 |
| FG.EC.DMBT1.ADH1C.1 | HDLBP | 0.49 | FG.Fibro.ADAMDEC1.FBLN1 | ADAMDEC1 | 4.40 | FG.T.TRDC.ZNF683 | ZNF683 | 1.16 |
| FG.EC.DMBT1.ADH1C.1 | ARHGEF35 | 0.32 | FG.Fibro.ADAMDEC1.FBLN1 | LUM | 3.73 | FG.T/NK.CCL4.GZMB | CCL4 | 3.36 |
| FG.EC.DMBT1.ADH1C.1 | ADGRG7 | 0.26 | FG.Fibro.ADAMDEC1.FBLN1 | FBLN1 | 3.38 | FG.T/NK.CCL4.GZMB | NKG7 | 2.68 |
| FG.EC.DMBT1.ADH1C.1 | MARVELD2 | 0.27 | FG.Fibro.ADAMDEC1.FBLN1 | DCN | 3.37 | FG.T/NK.CCL4.GZMB | CCL4L2 | 2.54 |
| FG.EC.DMBT1.ADH1C.1 | HEPH | 0.27 | FG.Fibro.ADAMDEC1.FBLN1 | C1S | 3.22 | FG.T/NK.CCL4.GZMB | GZMA | 2.43 |
| FG.EC.DMBT1.ADH1C.1 | SH3YL1 | 0.33 | FG.Fibro.ADAMDEC1.FBLN1 | CFH | 3.15 | FG.T/NK.CCL4.GZMB | CCL5 | 2.37 |
| FG.EC.DMBT1.ADH1C.1 | SDHA | 0.51 | FG.Fibro.ADAMDEC1.FBLN1 | MFAP4 | 3.06 | FG.T/NK.CCL4.GZMB | KLRD1 | 2.11 |
| FG.EC.DMBT1.ADH1C.1 | RP11-467L13.7 | 0.38 | FG.Fibro.ADAMDEC1.FBLN1 | C1R | 3.00 | FG.T/NK.CCL4.GZMB | TRDC | 2.05 |
| FG.EC.DMBT1.ADH1C.1 | RAB11FIP1 | 0.61 | FG.Fibro.ADAMDEC1.FBLN1 | CXCL12 | 2.79 | FG.T/NK.CCL4.GZMB | GZMK | 2.04 |
| FG.EC.DMBT1.ADH1C.1 | ACSS2 | 0.30 | FG.Fibro.ADAMDEC1.FBLN1 | COL6A2 | 2.71 | FG.T/NK.CCL4.GZMB | GZMB | 2.01 |
| FG.EC.DMBT1.ADH1C.1 | SLC25A1 | 0.43 | FG.Fibro.ADAMDEC1.FBLN1 | COL3A1 | 2.66 | FG.T/NK.CCL4.GZMB | CMC1 | 1.96 |
| FG.EC.DMBT1.ADH1C.1 | NDUFS4 | 0.48 | FG.Fibro.ADAMDEC1.FBLN1 | SPARC | 2.60 | FG.T/NK.CCL4.GZMB | XCL1 | 1.86 |
| FG.EC.DMBT1.ADH1C.1 | SLC25A24 | 0.28 | FG.Fibro.ADAMDEC1.FBLN1 | CFD | 2.53 | FG.T/NK.CCL4.GZMB | XCL2 | 1.85 |
| FG.EC.DMBT1.ADH1C.1 | COX5A | 0.94 | FG.Fibro.ADAMDEC1.FBLN1 | QSOX1 | 2.46 | FG.T/NK.CCL4.GZMB | CST7 | 1.84 |
| FG.EC.DMBT1.ADH1C.1 | ILVBL | 0.33 | FG.Fibro.ADAMDEC1.FBLN1 | ASPN | 2.45 | FG.T/NK.CCL4.GZMB | IFNG | 1.76 |
| FG.EC.DMBT1.ADH1C.1 | P4HB | 0.92 | FG.Fibro.ADAMDEC1.FBLN1 | IGFBP3 | 2.45 | FG.T/NK.CCL4.GZMB | CCL3 | 1.68 |
| FG.EC.DMBT1.ADH1C.1 | DNPH1 | 0.73 | FG.Fibro.ADAMDEC1.FBLN1 | ADH1B | 2.44 | FG.T/NK.CCL4.GZMB | CTSW | 1.62 |
| FG.EC.DMBT1.NPM1 | PPP1R1B | 1.12 | FG.Fibro.ADAMDEC1.FBLN1 | MMP2 | 2.43 | FG.T/NK.CCL4.GZMB | HCST | 1.56 |
| FG.EC.DMBT1.REG1A | DMBT1 | 2.59 | FG.Fibro.ADAMDEC1.FBLN1 | GSN | 2.41 | FG.T/NK.CCL4.GZMB | FCER1G | 1.51 |
| FG.EC.FABP6.CUBN | FABP6 | 5.33 | FG.Fibro.ADAMDEC1.FBLN1 | ANGPTL1 | 2.41 | FG.T/NK.CCL4.GZMB | TYROBP | 1.51 |
| FG.EC.FABP6.CUBN | PHGR1 | 3.60 | FG.Fibro.ADAMDEC1.FBLN1 | CXCL14 | 2.40 | FG.T/NK.CCL4.GZMB | CD7 | 1.48 |
| FG.EC.FABP6.CUBN | GUCA2A | 3.26 | FG.Fibro.ADAMDEC1.FBLN1 | EDIL3 | 2.35 | FG.T/NK.CCL4.GZMB | IL2RB | 1.47 |
| FG.EC.FABP6.CUBN | REG1B | 2.64 | FG.Fibro.ADAMDEC1.FBLN1 | FHL1 | 2.31 | FG.T/NK.CCL4.GZMB | KLRC1 | 1.44 |
| FG.EC.FABP6.CUBN | FABP2 | 2.59 | FG.Fibro.ADAMDEC1.FBLN1 | MEG3 | 2.28 | FG.T/NK.CCL4.GZMB | KLRB1 | 1.44 |
| FG.EC.FABP6.CUBN | KRT19 | 2.57 | FG.Fibro.ADAMDEC1.FBLN1 | ABCA8 | 2.28 | FG.T/NK.CCL4.GZMB | CD160 | 1.43 |
| FG.EC.FABP6.CUBN | ADIRF | 2.51 | FG.Fibro.ADAMDEC1.FBLN1 | GGT5 | 2.27 | FG.T/NK.CCL4.GZMB | PRF1 | 1.42 |
| FG.EC.FABP6.CUBN | DPEP1 | 2.50 | FG.Fibro.ADAMDEC1.FBLN1 | FILIP1L | 2.22 | FG.T/NK.CCL4.GZMB | KLRF1 | 1.42 |
| FG.EC.FABP6.CUBN | SERPINA1 | 2.47 | FG.Fibro.ADAMDEC1.FBLN1 | GPX3 | 2.22 | FG.T/NK.CCL4.GZMB | HOPX | 1.39 |
| FG.EC.FABP6.CUBN | FABP1 | 2.36 | FG.Fibro.ADAMDEC1.FBLN1 | TM4SF1 | 2.21 | FG.T/NK.CCL4.GZMB | DUSP2 | 1.34 |
| FG.EC.FABP6.CUBN | C15orf48 | 2.35 | FG.Fibro.ADAMDEC1.FBLN1 | COL1A2 | 2.15 | FG.T/NK.CCL4.GZMB | CD69 | 1.31 |
| FG.EC.FABP6.CUBN | KRT8 | 2.31 | FG.Fibro.ADAMDEC1.FBLN1 | VCAM1 | 2.12 | FG.T/NK.CCL4.GZMB | GZMM | 1.30 |
| FG.EC.FABP6.CUBN | GUCA2B | 2.30 | FG.Fibro.ADAMDEC1.FBLN1 | IGFBP7 | 2.12 | FG.T/NK.CCL4.GZMB | IFITM2 | 1.30 |
| FG.EC.FABP6.CUBN | PRAP1 | 2.24 | FG.Fibro.ADAMDEC1.FBLN1 | HAPLN1 | 2.10 | FG.T/NK.CCL4.GZMB | CLIC3 | 1.29 |
| FG.EC.FABP6.CUBN | ALDOB | 2.00 | FG.Fibro.ADAMDEC1.FBLN1 | EMILIN1 | 2.09 | FG.T/NK.CCL4.GZMB | SRGN | 1.12 |
| FG.EC.FABP6.CUBN | LGALS2 | 1.87 | FG.Fibro.ADAMDEC1.FBLN1 | PMP22 | 2.08 | FG.T/NK.CCL4.GZMB | HLA-B | 0.74 |
| FG.EC.FABP6.CUBN | FXYD3 | 1.81 | FG.Fibro.ADAMDEC1.FBLN1 | CALD1 | 2.07 | FG.T/NK.CCL4.GZMB | TMSB4X | 0.61 |
| FG.EC.FABP6.CUBN | KRT20 | 1.79 | FG.Fibro.ADAMDEC1.FBLN1 | C7 | 2.05 | FG.T/NK.CCL4.GZMB | B2M | 0.60 |
| FG.EC.FABP6.CUBN | CDKN2B-AS1 | 1.37 | FG.Fibro.ADAMDEC1.FBLN1 | FBLN5 | 2.04 | FG.T/NK.CCL4.GZMB | GZMH | 1.03 |
| FG.EC.FABP6.CUBN | TMIGD1 | 1.34 | FG.Fibro.ADAMDEC1.FBLN1 | PLTP | 2.03 | FG.T/NK/ILC.CCR7.SELL | CCR7 | 1.15 |
| FG.EC.FABP6.CUBN | ENPEP | 1.33 | FG.Fibro.ADAMDEC1.FBLN1 | APOE | 2.00 | FG.T/NK/ILC.CCR7.SELL | RPS10 | 0.98 |
| FG.EC.FABP6.CUBN | FAM151A | 1.19 | FG.Fibro.ADAMDEC1.FBLN1 | MXRA8 | 1.99 | FG.T/NK/ILC.CCR7.SELL | GIMAP7 | 0.98 |
| FG.EC.FABP6.CUBN | CUBN | 1.18 | FG.Fibro.ADAMDEC1.FBLN1 | TGFBI | 1.99 | FG.T/NK/ILC.CCR7.SELL | CD7 | 0.93 |
| FG.EC.FABP6.CUBN | LYPD8 | 0.96 | FG.Fibro.ADAMDEC1.FBLN1 | AZM | 1.96 | FG.T/NK/ILC.CCR7.SELL | SELL | 0.91 |
| FG.EC.FABP6.CUBN | TSPAN1 | 0.85 | FG.Fibro.ADAMDEC1.FBLN1 | SPARCL1 | 1.95 | FG.T/NK/ILC.CCR7.SELL | LDHB | 0.88 |
| FG.EC.FABP6.CUBN | CPO | 0.79 | FG.Fibro.ADAMDEC1.FBLN1 | CLEC14A | 1.94 | FG.T/NK/ILC.CCR7.SELL | RPS12 | 0.88 |
| FG.EC.FABP6.CUBN | RP3-417L20.4 | 0.69 | FG.Fibro.ADAMDEC1.FBLN1 | NKX2-3 | 1.91 | FG.T/NK/ILC.CCR7.SELL | LEF1 | 0.86 |
| FG.EC.GSTA2.AADAC | GSTA1 | 3.26 | FG.Fibro.ADAMDEC1.FBLN1 | CYGB | 1.90 | FG.T/NK/ILC.CCR7.SELL | CD3D | 0.86 |
| FG.EC.GSTA2.AADAC | FABP1 | 2.91 | FG.Fibro.ADAMDEC1.FBLN1 | VCAN | 1.84 | FG.T/NK/ILC.CCR7.SELL | RPS15A | 0.84 |
| FG.EC.GSTA2.AADAC | RBP2 | 2.77 | FG.Fibro.ADAMDEC1.FBLN1 | SERPING1 | 1.82 | FG.T/NK/ILC.CCR7.SELL | CD3G | 0.81 |
| FG.EC.GSTA2.AADAC | PIGR | 2.47 | FG.Fibro.ADAMDEC1.FBLN1 | SPON2 | 1.80 | FG.T/NK/ILC.CCR7.SELL | TRBC1 | 0.80 |
| FG.EC.GSTA2.AADAC | FABP2 | 2.45 | FG.Fibro.ADAMDEC1.FBLN1 | SNAI2 | 1.79 | FG.T/NK/ILC.CCR7.SELL | RPL36A | 0.78 |
| FG.EC.GSTA2.AADAC | LGALS4 | 2.25 | FG.Fibro.ADAMDEC1.FBLN1 | COL1A1 | 1.77 | FG.T/NK/ILC.CCR7.SELL | RPL30 | 0.78 |
| FG.EC.GSTA2.AADAC | EPCAM | 2.25 | FG.Fibro.ADAMDEC1.FBLN1 | CXCL6 | 1.75 | FG.T/NK/ILC.CCR7.SELL | RPL38 | 0.77 |
| FG.EC.GSTA2.AADAC | CES2 | 2.21 | FG.Fibro.ADAMDEC1.FBLN1 | PLAT | 1.75 | FG.T/NK/ILC.CCR7.SELL | RPL32 | 0.76 |
| FG.EC.GSTA2.AADAC | CBR1 | 2.19 | FG.Fibro.ADAMDEC1.FBLN1 | FN1 | 1.73 | FG.T/NK/ILC.CCR7.SELL | CD3E | 0.75 |
| FG.EC.GSTA2.AADAC | TSPAN8 | 2.18 | FG.Fibro.ADAMDEC1.FBLN1 | BMP5 | 1.71 | FG.T/NK/ILC.CCR7.SELL | RPS3A | 0.75 |
| FG.EC.GSTA2.AADAC | AKR1B10 | 2.15 | FG.Fibro.ADAMDEC1.FBLN1 | BCHE | 1.70 | FG.T/NK/ILC.CCR7.SELL | RPL9 | 0.72 |
| FG.EC.GSTA2.AADAC | ALDOB | 2.14 | FG.Fibro.ADAMDEC1.FBLN1 | RARRES2 | 1.65 | FG.T/NK/ILC.CCR7.SELL | RPL31 | 0.72 |
| FG.EC.GSTA2.AADAC | KRT8 | 2.11 | FG.Fibro.ADAMDEC1.FBLN1 | LOXL1 | 1.64 | FG.T/NK/ILC.CCR7.SELL | RPL22 | 0.72 |
| FG.EC.GSTA2.AADAC | SMIM24 | 2.05 | FG.Fibro.ADAMDEC1.FBLN1 | DKK3 | 1.64 | FG.T/NK/ILC.CCR7.SELL | RPS13 | 0.71 |
| FG.EC.GSTA2.AADAC | PTGR1 | 2.01 | FG.Fibro.ADAMDEC1.FBLN1 | SDC2 | 1.64 | FG.T/NK/ILC.CCR7.SELL | RPS25 | 0.71 |
| FG.EC.GSTA2.AADAC | ADH1C | 1.99 | FG.Fibro.ADAMDEC1.FBLN1 | SGCE | 1.63 | FG.T/NK/ILC.CCR7.SELL | RPLP2 | 0.71 |
| FG.EC.GSTA2.AADAC | MGST3 | 1.96 | FG.Fibro.ADAMDEC1.FBLN1 | CCL11 | 1.60 | FG.T/NK/ILC.CCR7.SELL | RPL34 | 0.70 |
| FG.EC.GSTA2.AADAC | HSD17B2 | 1.93 | FG.Fibro.ADAMDEC1.FBLN1 | TCF21 | 1.59 | FG.T/NK/ILC.CCR7.SELL | RPL35A | 0.70 |
| FG.EC.GSTA2.AADAC | ANPEP | 1.85 | FG.Fibro.ADAMDEC1.FBLN1 | FOXF1 | 1.55 | FG.T/NK/ILC.CCR7.SELL | RPS27 | 0.69 |
| FG.EC.GSTA2.AADAC | SI | 1.82 | FG.Fibro.ADAMDEC1.FBLN1 | STMN2 | 1.54 | FG.T/NK/ILC.CCR7.SELL | RPS21 | 0.69 |
| FG.EC.GSTA2.AADAC | PCK1 | 1.81 | FG.Fibro.ADAMDEC1.FBLN1 | EFEMP1 | 1.50 | FG.T/NK/ILC.CCR7.SELL | RPL27 | 0.69 |
| FG.EC.GSTA2.AADAC | AKR7A3 | 1.80 | FG.Fibro.ADAMDEC1.FBLN1 | COL12A1 | 1.50 | FG.T/NK/ILC.CCR7.SELL | RPL37 | 0.69 |
| FG.EC.GSTA2.AADAC | FBP1 | 1.79 | FG.Fibro.ADAMDEC1.FBLN1 | FNDC1 | 1.39 | FG.T/NK/ILC.CCR7.SELL | RPS16 | 0.68 |
| FG.EC.GSTA2.AADAC | OAT | 1.79 | FG.Fibro.ADAMDEC1.FBLN1 | ALDH1A3 | 1.33 | FG.T/NK/ILC.CCR7.SELL | RPL21 | 0.68 |
| FG.EC.GSTA2.AADAC | CYB5A | 1.78 | FG.Fibro.ADAMDEC1.FBLN1 | DDR2 | 1.54 | FG.T/NK/ILC.CCR7.SELL | RPS17 | 0.65 |
| FG.EC.GSTA2.AADAC | CMBL | 1.77 | FG.Fibro.ADAMDEC1.FBLN1 | COL6A5 | 1.16 | FG.T/NK/ILC.CCR7.SELL | RPS27A | 0.65 |
| FG.EC.GSTA2.AADAC | CCL25 | 1.77 | FG.Fibro.ADAMDEC1.FBLN1 | OLFML3 | 1.42 | FG.T/NK/ILC.CCR7.SELL | TRAC | 0.64 |
| FG.EC.GSTA2.AADAC | SLC26A3 | 1.76 | FG.Fibro.ADAMDEC1.FBLN1 | PTN | 1.54 | FG.T/NK/ILC.CCR7.SELL | RPL39 | 0.64 |
| FG.EC.GSTA2.AADAC | GSTA2 | 1.75 | FG.Fibro.C3.FDCSP | C3 | 3.34 | FG.T/NK/ILC.CCR7.SELL | RPS6 | 0.64 |
| FG.EC.GSTA2.AADAC | AKR1C3 | 1.73 | FG.Fibro.CCL11.FABP5 | CCL11 | 3.65 | FG.T/NK/ILC.CCR7.SELL | RPS29 | 0.64 |
| FG.EC.GSTA2.AADAC | CLDN4 | 1.70 | FG.Fibro.CCL11.FABP5 | LUM | 3.60 | FG.T/NK/ILC.CCR7.SELL | RPS14 | 0.63 |
| FG.EC.GSTA2.AADAC | GATM | 1.69 | FG.Fibro.CCL11.FABP5 | CFD | 3.55 | FG.T/NK/ILC.CCR7.SELL | RPL14 | 0.63 |
| FG.EC.GSTA2.AADAC | SLC5A1 | 1.68 | FG.Fibro.CCL11.FABP5 | ADAMDEC1 | 3.39 | FG.T/NK/ILC.CCR7.SELL | RPS3 | 0.62 |
| FG.EC.GSTA2.AADAC | CLDN3 | 1.68 | FG.Fibro.CCL11.FABP5 | MFAP4 | 3.15 | FG.T/NK/ILC.CCR7.SELL | RPL13 | 0.62 |
| FG.EC.GSTA2.AADAC | LGALS3 | 1.67 | FG.Fibro.CCL11.FABP5 | DCN | 3.07 | FG.T/NK/ILC.CCR7.SELL | RPS20 | 0.62 |
| FG.EC.GSTA2.AADAC | PEBP1 | 1.65 | FG.Fibro.CCL11.FABP5 | FBLN1 | 2.97 | FG.T/NK/ILC.CCR7.SELL | RPL11 | 0.61 |
| FG.EC.GSTA2.AADAC | MTTP | 1.62 | FG.Fibro.CCL11.FABP5 | IGFBP7 | 2.73 | FG.T/NK/ILC.CCR7.SELL | RPL19 | 0.60 |
| FG.EC.GSTA2.AADAC | KHK | 1.61 | FG.Fibro.CCL11.FABP5 | C1S | 2.66 | FG.T/NK/ILC.CCR7.SELL | RPS23 | 0.60 |
| FG.EC.GSTA2.AADAC | APOB | 1.59 | FG.Fibro.CCL11.FABP5 | ADH1B | 2.50 | FG.T/NK/ILC.CCR7.SELL | TPT1 | 0.59 |
| FG.EC.GSTA2.AADAC | KRT19 | 1.59 | FG.Fibro.CCL11.FABP5 | C1R | 2.47 | FG.T/NK/ILC.CCR7.SELL | RPS8 | 0.59 |
| FG.EC.GSTA2.AADAC | ALDH1A1 | 1.59 | FG.Fibro.CCL11.FABP5 | RARRES2 | 2.43 | FG.T/NK/ILC.CCR7.SELL | RPS28 | 0.59 |
| FG.EC.GSTA2.AADAC | AGR3 | 1.57 | FG.Fibro.CCL11.FABP5 | IFITM3 | 2.41 | FG.T/NK/ILC.CCR7.SELL | RPL10 | 0.58 |
| FG.EC.GSTA2.AADAC | TMPRSS15 | 1.56 | FG.Fibro.CCL11.FABP5 | CCL2 | 2.38 | FG.T/NK/ILC.CCR7.SELL | RPL26 | 0.58 |
| FG.EC.GSTA2.AADAC | CA2 | 1.55 | FG.Fibro.CCL11.FABP5 | APOE | 2.37 | FG.T/NK/ILC.CCR7.SELL | RPL23A | 0.56 |
| FG.EC.GSTA2.AADAC | KRT18 | 1.55 | FG.Fibro.CCL11.FABP5 | CFH | 2.36 | FG.T/NK/ILC.CCR7.SELL | RPL5 | 0.55 |
| FG.EC.GSTA2.AADAC | MT1G | 1.53 | FG.Fibro.CCL11.FABP5 | COL3A1 | 2.33 | FG.T/NK/ILC.CCR7.SELL | RPL37A | 0.55 |
| FG.EC.GSTA2.AADAC | ATP5G3 | 1.53 | FG.Fibro.CCL11.FABP5 | LGALS1 | 2.28 | FG.T/NK/ILC.CCR7.SELL | RPL10A | 0.55 |
| FG.EC.GSTA2.AADAC | MME | 1.52 | FG.Fibro.CCL11.FABP5 | GSN | 2.26 | FG.T/NK/ILC.CCR7.SELL | RPL4 | 0.54 |
| FG.EC.GSTA2.AADAC | ATP1B1 | 1.51 | FG.Fibro.CCL11.FABP5 | TMEM176B | 2.23 | FG.T/NK/ILC.CCR7.SELL | RPSA | 0.54 |
| FG.EC.GSTA2.AADAC | PHGR1 | 1.51 | FG.Fibro.CCL11.FABP5 | PTGDS | 2.18 | FG.T/NK/ILC.CCR7.SELL | RPL41 | 0.53 |
| FG.EC.GSTA2.AADAC | CLDN7 | 1.51 | FG.Fibro.CCL11.FABP5 | CALD1 | 2.16 | FG.T/NK/ILC.CCR7.SELL | RPL7 | 0.53 |
| FG.EC.GSTA2.AADAC | ADIRF | 1.50 | FG.Fibro.CCL11.FABP5 | GPX3 | 2.15 | FG.T/NK/ILC.CCR7.SELL | RPL13A | 0.52 |
| FG.EC.GSTA2.AADAC | UGT2B17 | 1.50 | FG.Fibro.CCL11.FABP5 | CXCL12 | 2.15 | FG.T/NK/ILC.CCR7.SELL | UBA52 | 0.51 |
| FG.EC.GSTA2.AADAC | AADAC | 1.48 | FG.Fibro.CCL11.FABP5 | COL1A2 | 2.13 | FG.T/NK/ILC.CCR7.SELL | RPL24 | 0.51 |
| FG.EC.GSTA2.AADAC | DHRS11 | 1.48 | FG.Fibro.CCL11.FABP5 | COL6A2 | 2.08 | FG.T/NK/ILC.CCR7.SELL | RPL12 | 0.51 |
| FG.EC.GSTA2.AADAC | SUCLG1 | 1.47 | FG.Fibro.CCL11.FABP5 | FABP5 | 2.06 | FG.T/NK/ILC.CCR7.SELL | RPS5 | 0.51 |
| FG.EC.GSTA2.AADAC | DNASE1 | 1.45 | FG.Fibro.CCL11.FABP5 | A2M | 2.05 | FG.T/NK/ILC.CCR7.SELL | RPS4X | 0.50 |
| FG.EC.GSTA2.AADAC | S100A14 | 1.44 | FG.Fibro.CCL11.FABP5 | PPP1R14A | 2.04 | FG.T/NK/ILC.CCR7.SELL | RPL18 | 0.50 |
| FG.EC.GSTA2.AADAC | SULT1A1 | 1.42 | FG.Fibro.CCL11.FABP5 | PLTP | 2.03 | FG.T/NK/ILC.CCR7.SELL | TMSB10 | 0.49 |
| FG.EC.GSTA2.AADAC | ELF3 | 1.42 | FG.Fibro.CCL11.FABP5 | CXCL14 | 2.03 | FG.T/NK/ILC.CCR7.SELL | EEF1A1 | 0.49 |
| FG.EC.GSTA2.AADAC | CYP3A4 | 1.42 | FG.Fibro.CCL11.FABP5 | TCF21 | 2.01 | FG.T/NK/ILC.CCR7.SELL | RPL3 | 0.49 |
| FG.EC.GSTA2.AADAC | MGST2 | 1.40 | FG.Fibro.CCL11.FABP5 | IGFBP3 | 1.99 | FG.T/NK/ILC.CCR7.SELL | RPL6 | 0.48 |
| FG.EC.GSTA2.AADAC | ATP1A1 | 1.38 | FG.Fibro.CCL11.FABP5 | CD63 | 1.96 | FG.T/NK/ILC.CCR7.SELL | RPS18 | 0.48 |
| FG.EC.GSTA2.AADAC | PRAP1 | 1.38 | FG.Fibro.CCL11.FABP5 | PLAC9 | 1.94 | FG.T/NK/ILC.CCR7.SELL | RPL28 | 0.47 |
| FG.EC.GSTA2.AADAC | CLDN15 | 1.38 | FG.Fibro.CCL11.FABP5 | HAPLN1 | 1.92 | FG.T/NK/ILC.CCR7.SELL | RPS24 | 0.46 |
| FG.EC.GSTA2.AADAC | PCK2 | 1.37 | FG.Fibro.CCL11.FABP5 | CTSC | 1.91 | FG.T/NK/ILC.CCR7.SELL | RPS2 | 0.46 |
| FG.EC.GSTA2.AADAC | KRT20 | 1.36 | FG.Fibro.CCL11.FABP5 | LTBP4 | 1.89 | FG.T/NK/ILC.CCR7.SELL | RPL27A | 0.46 |
| FG.EC.GSTA2.AADAC | PPP1R16A | 1.35 | FG.Fibro.CCL11.FABP5 | QSOX1 | 1.89 | FG.T/NK/ILC.CCR7.SELL | MALAT1 | 0.45 |
| FG.EC.GSTA2.AADAC | TXN | 1.34 | FG.Fibro.CCL11.FABP5 | RBP1 | 1.80 | FG.T/NK/ILC.CCR7.SELL | RPL18A | 0.45 |
| FG.EC.GSTA2.AADAC | HSPB1 | 1.31 | FG.Fibro.CCL11.FABP5 | TMEM176A | 1.79 | FG.T/NK/ILC.CCR7.SELL | RPS15 | 0.44 |
| FG.EC.GSTA2.AADAC | REG1A | 1.30 | FG.Fibro.CCL11.FABP5 | SPARCL1 | 1.79 | FG.T/NK/ILC.CCR7.SELL | RPL29 | 0.44 |
| FG.EC.GSTA2.AADAC | ID1 | 1.30 | FG.Fibro.CCL11.FABP5 | SPARC | 1.79 | FG.T/NK/ILC.CCR7.SELL | RPL36 | 0.43 |
| FG.EC.GSTA2.AADAC | TKFC | 1.28 | FG.Fibro.CCL11.FABP5 | MMP2 | 1.78 | FG.T/NK/ILC.CCR7.SELL | RPS7 | 0.43 |
| FG.EC.GSTA2.AADAC | SLC2A2 | 1.28 | FG.Fibro.CCL11.FABP5 | PROCR | 1.77 | FG.T/NK/ILC.CCR7.SELL | RPS9 | 0.43 |
| FG.EC.GSTA2.AADAC | PRDX1 | 1.27 | FG.Fibro.CCL11.FABP5 | PMP22 | 1.76 | FG.T/NK/ILC.CCR7.SELL | RPS19 | 0.42 |
| FG.EC.GSTA2.AADAC | HEBP1 | 1.25 | FG.Fibro.CCL11.FABP5 | SPON2 | 1.73 | FG.T/NK/ILC.CCR7.SELL | RPL35 | 0.39 |
| FG.EC.GSTA2.AADAC | TM4SF5 | 1.25 | FG.Fibro.CCL11.FABP5 | TM4SF1 | 1.72 | FG.T/NK/ILC.GNLY.TYROBP | GNLY | 3.79 |
| FG.EC.GSTA2.AADAC | CPS1 | 1.25 | FG.Fibro.CCL11.FABP5 | IGFBP6 | 1.72 | FG.T/NK/ILC.GNLY.TYROBP | NKG7 | 2.01 |
| FG.EC.GSTA2.AADAC | HMGCS2 | 1.24 | FG.Fibro.CCL11.FABP5 | EDIL3 | 1.72 | FG.T/NK/ILC.GNLY.TYROBP | GZMB | 1.90 |
| FG.EC.GSTA2.AADAC | PSME2 | 1.22 | FG.Fibro.CCL11.FABP5 | GGT5 | 1.70 | FG.T/NK/ILC.GNLY.TYROBP | TRDC | 1.70 |
| FG.EC.GSTA2.AADAC | AMN | 1.22 | FG.Fibro.CCL11.FABP5 | CXCL1 | 1.70 | FG.T/NK/ILC.GNLY.TYROBP | KLRD1 | 1.64 |
| FG.EC.GSTA2.AADAC | FCGRT | 1.22 | FG.Fibro.CCL11.FABP5 | ASPN | 1.70 | FG.T/NK/ILC.GNLY.TYROBP | KLRB1 | 1.48 |
| FG.EC.GSTA2.AADAC | ANXA4 | 1.21 | FG.Fibro.CCL11.FABP5 | PDLIM1 | 1.68 | FG.T/NK/ILC.GNLY.TYROBP | CTSW | 1.47 |
| FG.EC.GSTA2.AADAC | CYSTM1 | 1.20 | FG.Fibro.CCL11.FABP5 | EMILIN1 | 1.68 | FG.T/NK/ILC.GNLY.TYROBP | CD7 | 1.41 |
| FG.EC.GSTA2.AADAC | MAOA | 1.19 | FG.Fibro.CCL11.FABP5 | TIMP1 | 1.68 | FG.T/NK/ILC.GNLY.TYROBP | KRT86 | 1.41 |
| FG.EC.GSTA2.AADAC | SDCBP2 | 1.19 | FG.Fibro.CCL11.FABP5 | CYGB | 1.67 | FG.T/NK/ILC.GNLY.TYROBP | TYROBP | 1.30 |
| FG.EC.GSTA2.AADAC | SLC25A5 | 1.18 | FG.Fibro.CCL11.FABP5 | S100A13 | 1.67 | FG.T/NK/ILC.GNLY.TYROBP | KLRC1 | 1.27 |
| FG.EC.GSTA2.AADAC | CRYL1 | 1.18 | FG.Fibro.CCL11.FABP5 | COL1A1 | 1.66 | FG.T/NK/ILC.GNLY.TYROBP | KRT81 | 1.06 |
| FG.EC.GSTA2.AADAC | LIMA1 | 1.18 | FG.Fibro.CCL11.FABP5 | S100A6 | 1.65 | FG.T/NK/ILC.MAF.RPS26 | TRBC1 | 1.48 |
| FG.EC.GSTA2.AADAC | C19orf33 | 1.17 | FG.Fibro.CCL11.FABP5 | FHL1 | 1.65 | FG.T/NK/ILC.MAF.RPS26 | TRBC2 | 1.31 |
| FG.EC.GSTA2.AADAC | COX5B | 1.17 | FG.Fibro.CCL11.FABP5 | NGFRAP1 | 1.65 | FG.T/NK/ILC.MAF.RPS26 | FYB | 1.30 |
| FG.EC.GSTA2.AADAC | MPST | 1.16 | FG.Fibro.CCL11.FABP5 | TNC | 1.63 | FG.T/NK/ILC.MAF.RPS26 | CD3D | 1.30 |
| FG.EC.GSTA2.AADAC | DSP | 1.16 | FG.Fibro.CCL11.FABP5 | SERPING1 | 1.63 | FG.T/NK/ILC.MAF.RPS26 | CD2 | 1.27 |
| FG.EC.GSTA2.AADAC | AGR2 | 1.15 | FG.Fibro.CCL11.FABP5 | CXCL6 | 1.61 | FG.T/NK/ILC.MAF.RPS26 | TRAC | 1.27 |
| FG.EC.GSTA2.AADAC | VIL1 | 1.15 | FG.Fibro.CCL11.FABP5 | PTN | 1.60 | FG.T/NK/ILC.MAF.RPS26 | KLRB1 | 1.21 |
| FG.EC.GSTA2.AADAC | PBLD | 1.15 | FG.Fibro.CCL11.FABP5 | PLPP1 | 1.58 | FG.T/NK/ILC.MAF.RPS26 | TIGIT | 1.14 |
| FG.EC.GSTA2.AADAC | ANXA2 | 1.15 | FG.Fibro.CCL11.FABP5 | LAPTM4A | 1.57 | FG.T/NK/ILC.MAF.RPS26 | IL32 | 1.05 |
| FG.EC.GSTA2.AADAC | ADA | 1.15 | FG.Fibro.CCL11.FABP5 | SELM | 1.57 | FG.T/NK/ILC.MAF.RPS26 | ICOS | 1.00 |
| FG.EC.GSTA2.AADAC | SMIM22 | 1.14 | FG.Fibro.CCL11.FABP5 | LGALS3BP | 1.57 | FG.T/NK/ILC.MAF.RPS26 | SRGN | 0.99 |
| FG.EC.GSTA2.AADAC | HSPA1B | 1.14 | FG.Fibro.CCL11.FABP5 | NUPR1 | 1.55 | FG.T/NK/ILC.MAF.RPS26 | CD3E | 0.96 |
| FG.EC.GSTA2.AADAC | UQCRQ | 1.13 | FG.Fibro.CCL11.FABP5 | VIM | 1.54 | FG.T/NK/ILC.MAF.RPS26 | PTPRC | 0.88 |
| FG.EC.GSTA2.AADAC | COX5A | 1.13 | FG.Fibro.CCL11.FABP5 | CLEC11A | 1.54 | FG.T/NK/ILC.MAF.RPS26 | CD7 | 0.77 |
| FG.EC.GSTA2.AADAC | COX7B | 1.13 | FG.Fibro.CCL11.FABP5 | VCAN | 1.54 | FG.T/NK/ILC.MAF.RPS26 | MALAT1 | 0.69 |
| FG.EC.GSTA2.AADAC | ACSL5 | 1.13 | FG.Fibro.CCL11.FABP5 | ANGPTL1 | 1.53 | FG.T/NK/ILC.MAF.RPS26 | HLA-A | 0.58 |
| FG.EC.GSTA2.AADAC | CHP2 | 1.12 | FG.Fibro.CCL11.FABP5 | FILIP1L | 1.53 | FG.T/NK/ILC.MAF.RPS26 | RPS29 | 0.56 |
| FG.EC.GSTA2.AADAC | P4HB | 1.12 | FG.Fibro.CCL11.FABP5 | SDC2 | 1.49 | FG.T/NK/ILC.MAF.RPS26 | B2M | 0.42 |
| FG.EC.GSTA2.AADAC | FAM3B | 1.12 | FG.Fibro.CCL11.FABP5 | IFI27 | 1.48 | FG.T/NK/ILC.MAF.RPS26 | RPS27 | 0.41 |
| FG.EC.GSTA2.AADAC | TM4SF20 | 1.12 | FG.Fibro.CCL11.FABP5 | MYL9 | 1.48 | FG.T/NK/ILC.MAF.RPS26 | RPS15A | 0.38 |
| FG.EC.GSTA2.AADAC | AGPAT2 | 1.11 | FG.Fibro.CCL11.FABP5 | VCAM1 | 1.48 | FG.T/NK/ILC.MAF.RPS26 | RPS4X | 0.35 |
| FG.EC.GSTA2.AADAC | SLC39A5 | 1.11 | FG.Fibro.CCL11.FABP5 | C7 | 1.47 | FG.T/NK/ILC.MAF.RPS26 | RPL34 | 0.35 |
| FG.EC.GSTA2.AADAC | DMBT1 | 1.10 | FG.Fibro.CCL11.FABP5 | SERPINF1 | 1.47 | FG.T/NK/ILC.MAF.RPS26 | RPL32 | 0.34 |
| FG.EC.GSTA2.AADAC | MALL | 1.09 | FG.Fibro.CCL11.FABP5 | FXYD1 | 1.46 | FG.T/NK/ILC.MAF.RPS26 | TMSB4X | 0.31 |
| FG.EC.GSTA2.AADAC | CDHR5 | 1.09 | FG.Fibro.CCL11.FABP5 | PLPP3 | 1.46 | FG.T/NK/ILC.MAF.RPS26 | RPS25 | 0.36 |
| FG.EC.GSTA2.AADAC | DDT | 1.09 | FG.Fibro.CCL11.FABP5 | COL6A3 | 1.45 | FG.T/NK/ILC.MAF.RPS26 | GIMAP7 | 0.92 |
| FG.EC.GSTA2.AADAC | CDH17 | 1.08 | FG.Fibro.CCL11.FABP5 | LINC01082 | 1.45 | FG.Tclls.JUN.FOS | CD69 | 1.91 |
| FG.EC.GSTA2.AADAC | RNF186 | 1.08 | FG.Fibro.CCL11.FABP5 | MEG3 | 1.45 | FG.Tclls.JUN.FOS | IL7R | 1.80 |
| FG.EC.GSTA2.AADAC | PRSS3 | 1.08 | FG.Fibro.CCL11.FABP5 | RAB13 | 1.44 | FG.Tclls.JUN.FOS | ANXA1 | 1.79 |
| FG.EC.GSTA2.AADAC | MT-ND4L | 1.06 | FG.Fibro.CCL11.FABP5 | CCDC80 | 1.44 | FG.Tclls.JUN.FOS | CCL5 | 1.58 |
| FG.EC.GSTA2.AADAC | HPGD | 1.06 | FG.Fibro.CCL11.FABP5 | ANXA2 | 1.43 | FG.Tclls.JUN.FOS | FOS | 1.50 |
| FG.EC.GSTA2.AADAC | ATP5B | 1.06 | FG.Fibro.CCL11.FABP5 | COL6A1 | 1.43 | FG.Tclls.JUN.FOS | TSC22D3 | 1.47 |
| FG.EC.GSTA2.AADAC | DGAT1 | 1.05 | FG.Fibro.CCL11.FABP5 | PTMS | 1.43 | FG.Tclls.JUN.FOS | MYADM | 1.45 |
| FG.EC.GSTA2.AADAC | MUC13 | 1.05 | FG.Fibro.CCL11.FABP5 | SNAI2 | 1.42 | FG.Tclls.JUN.FOS | TNFAIP3 | 1.44 |
| FG.EC.GSTA2.AADAC | HADHB | 1.04 | FG.Fibro.CCL11.FABP5 | TNXB | 1.41 | FG.Tclls.JUN.FOS | TRAC | 1.36 |
| FG.EC.GSTA2.AADAC | MGST1 | 1.04 | FG.Fibro.CCL11.FABP5 | GNG11 | 1.39 | FG.Tclls.JUN.FOS | PTPRC | 1.27 |
| FG.EC.GSTA2.AADAC | UGT2A3 | 1.04 | FG.Fibro.CCL11.FABP5 | CLEC14A | 1.38 | FG.Tclls.JUN.FOS | CD2 | 1.26 |
| FG.EC.GSTA2.AADAC | ATP5O | 1.03 | FG.Fibro.CCL11.FABP5 | PCOLCE | 1.38 | FG.Tclls.JUN.FOS | KLRB1 | 1.23 |
| FG.EC.GSTA2.AADAC | ANXA13 | 1.03 | FG.Fibro.CCL11.FABP5 | TPPP3 | 1.36 | FG.Tclls.JUN.FOS | HSPA8 | 1.18 |
| FG.EC.GSTA2.AADAC | SULT2A1 | 1.03 | FG.Fibro.CCL11.FABP5 | ABCA8 | 1.36 | FG.Tclls.JUN.FOS | SARAF | 1.17 |
| FG.EC.GSTA2.AADAC | COX6A1 | 1.03 | FG.Fibro.CCL11.FABP5 | SGCE | 1.36 | FG.Tclls.JUN.FOS | DDX5 | 1.14 |
| FG.EC.GSTA2.AADAC | ZFAND2A | 1.03 | FG.Fibro.CCL11.FABP5 | FBLN5 | 1.35 | FG.Tclls.JUN.FOS | PABPC1 | 1.09 |
| FG.EC.GSTA2.AADAC | CYP3A5 | 1.02 | FG.Fibro.CCL11.FABP5 | FN1 | 1.35 | FG.Tclls.JUN.FOS | CXCR4 | 1.02 |
| FG.EC.GSTA2.AADAC | CYC1 | 1.01 | FG.Fibro.CCL11.FABP5 | NBL1 | 1.33 | FG.Tclls.JUN.FOS | UBC | 1.00 |
| FG.EC.GSTA2.AADAC | KLF5 | 1.01 | FG.Fibro.CCL11.FABP5 | PRKCDBP | 1.33 | FG.Tclls.JUN.FOS | HLA-C | 0.95 |
| FG.EC.GSTA2.AADAC | FOLH1 | 1.01 | FG.Fibro.CCL11.FABP5 | SOD3 | 1.33 | FG.Tclls.JUN.FOS | HLA-A | 0.80 |
| FG.EC.GSTA2.AADAC | ATP5G1 | 1.01 | FG.Fibro.CCL11.FABP5 | TPM2 | 1.33 | FG.Tclls.JUN.FOS | HLA-B | 0.71 |
| FG.EC.GSTA2.AADAC | MT2A | 1.01 | FG.Fibro.CCL11.FABP5 | CCL8 | 1.30 | FG.Tclls.JUN.FOS | ACTB | 0.61 |
| FG.EC.GSTA2.AADAC | CISD3 | 1.01 | FG.Fibro.CCL11.FABP5 | CCL13 | 1.30 | FG.Tclls.JUN.FOS | EEF1A1 | 0.59 |
| FG.EC.GSTA2.AADAC | MS4A8 | 1.00 | FG.Fibro.CCL11.FABP5 | CTSK | 1.30 | FG.Tclls.JUN.FOS | B2M | 0.46 |
| FG.EC.GSTA2.AADAC | COX4I1 | 1.00 | FG.Fibro.CCL11.FABP5 | EPHX1 | 1.30 | FG.Tclls.JUN.FOS | CD3E | 1.09 |
| FG.EC.GSTA2.AADAC | ATP5C1 | 0.99 | FG.Fibro.CCL11.FABP5 | WBP5 | 1.29 | FG.Tuft.GNG13.SH2D6 | AZGP1 | 2.82 |
| FG.EC.GSTA2.AADAC | MT-CO3 | 0.99 | FG.Fibro.CCL11.FABP5 | DPT | 1.29 | FG.Tuft.GNG13.SH2D6 | SH2D6 | 2.74 |
| FG.EC.GSTA2.AADAC | BDH2 | 0.99 | FG.Fibro.CCL11.FABP5 | TGFBI | 1.26 | FG.Tuft.GNG13.SH2D6 | TRPM5 | 2.18 |
| FG.EC.GSTA2.AADAC | PRDX2 | 0.99 | FG.Fibro.CCL11.FABP5 | TFPI | 1.25 | FG.Tuft.GNG13.SH2D6 | BMX | 2.01 |
| FG.EC.GSTA2.AADAC | UQCRC1 | 0.99 | FG.Fibro.CCL11.FABP5 | SFTA1P | 1.25 | FG.Tuft.GNG13.SH2D6 | GNG13 | 1.83 |
| FG.EC.GSTA2.AADAC | SULT1B1 | 0.99 | FG.Fibro.CCL11.FABP5 | NKX2-3 | 1.24 | FG.Tuft.GNG13.SH2D6 | POU2F3 | 1.56 |
| FG.EC.GSTA2.AADAC | HADHA | 0.98 | FG.Fibro.CCL11.FABP5 | COX7A1 | 1.23 | FG.Tuft.GNG13.SH2D6 | HTR3E | 1.24 |
| FG.EC.GSTA2.AADAC | MISP | 0.98 | FG.Fibro.CCL11.FABP5 | THY1 | 1.22 | FG.Tuft.GNG13.SH2D6 | GNAT3 | 1.09 |

TABLE 5
Demographics and clinical characteristics of patients analyzed on PREDICT.

| | | |
|---|---|---|
| Crohn's Disease | FGID | |
| n = 14 | n = 13 | P-values |

Demographics

| | | | | | |
|---|---|---|---|---|---|
| Age at diagnosis | 12.5 | (9-19) | 16 | (10-18) | 0.096 |
| (year) | | | | | |
| Female sex | 47% | (9 patients) | 46% | (6 patients) | >0.99 |
| Caucasian | 79% | (11 patients) | 84% | (11 patients) | |
| African American | 0% | (0 patients) | 8% | (1 patient) | |
| Native American | 0% | (0 patients) | 0% | (0 patients) | |
| or Alaskan Native | | | | | |
| Asian | 7% | (1 patient) | 0% | (0 patients) | |
| Mixed race | 14% | (2 patients) | 0% | (0 patients) | |
| Other | 0% | (0 patients) | 8% | (1 patient) | |

Growth

| | | | |
|---|---|---|---|
| Weight Z score | −0.61 | 0.12 | 0.12 |
| | (−3.31 to 2.31) | (−1.34 to 2.54) | |
| Height Z score | 0.09 | 0.01 | 0.86 |
| | (−2.04 to 2.32) | (−1.82 to 2.3) | |
| BMI Z score | −0.44 | 0.2 | 0.23 |
| | (−3.74 to 2.1) | (−2.15 to 2.4) | |

Disease location/activity
Location

| | | |
|---|---|---|
| L1 | 0% | (0 patients) |
| L2 | 0% | (0 patients) |
| L3 | 93% | (13 patient) |
| P modifier | 14% | (2 patients) |

Behavior

| | | |
|---|---|---|
| B1 | 93% | (13 patient) |
| B2 | 0% | (0 patients) |
| B3 | 0% | (0 patients) |

Severity in wPCDAI

Laboratory evaluation

| | | | |
|---|---|---|---|
| CRP (mg/dl) | 3.9 ± 0.8 | 0.7 ± 0.1 | 0.0019* |
| | (0.8 to 8) | (0.1 to 1) | |
| ESR (mm/hr) | 35.8 ± 6.2 | 5.2 ± 0.9 | <0.0001* |
| | (7 to 69) | (2 to 8) | |
| Hgb (g/dl) | 11.2 ± 0.5 | 13.1 ± 0.5 | 0.0018* |
| | (8.3 to 13.7) | (9.4 to 15.5) | |
| PLT (k/mm3) | 424.4 ± 38.8 | 274.2 ± 19.3 | 0.0213* |
| | (176 to 704) | (186 to 433) | |
| WBC (k/mm3) | 7.1 ± 0.4 | 7.1 ± 0.6 | 0.914 |
| | (4.6 to 10.1) | (4.2 to 11.5) | |
| Albumin (g/dl) | 3.5 ± 0.2 | 4.5 ± 0.1 | 0.0023* |
| | (2.5 to 4.6) | (4.3 to 5) | |

*indicates significance detected between Crohn's Disease (CD) and Functional Gastrointestinal Disorder (FGID).

TABLE 6
Demographics and clinical characteristics of Crohn's Disease cohorts.

| | | |
|---|---|---|
| Partial Responder | Full Responder | Not on anti-TNF |
| n = 5 | n = 5 | n = 4 |

Demographics and follow-up

| | | | |
|---|---|---|---|
| Age at diagnosis (year) | 12 (10 to 14) | 12 (9 to 14) | 15.5 (9-19) |
| Female sex | 40% (2 patients) | 40% (2 patients) | 50% (2 patients) |
| Caucasian | 80% (4 patients) | 80% (4 patients) | 50% (2 patients) |
| African American | 0% (0 patient) | 0% (0 patient) | 0% (0 patient) |
| Native American or | 0% (0 patient) | 0% (0 patient) | 0% (0 patient) |
| Alaskan Native | | | |
| Asian | 0% (0 patient) | 0% (0 patient) | 0% (0 patient) |
| Mixed race | 20% (1 patient) | 0% (0 patient) | 25% (1 patient) |
| Other | 0% (0 patient) | 20% (1 patient) | 0% (0 patient) |

Disease location/activity

| Location | | | |
|---|---|---|---|
| L1 | 0% (0 patient) | 0% (0 patient) | 0% (0 patient) |
| L2 | 0% (0 patient) | 0% (0 patient) | 0% (0 patient) |
| L3 | 100% (5 patients) | 100% (5 patients) | 75% (3 patients) |
| P modifier | 20% (1 patient) | 0% (0 patients) | 25% (1 patient) |
| Behavior | | | |
| B1 | 100% (5 patients) | 100% (5 patients) | 75% (3 patients) |
| B2 | 0% (0 patients) | 0% (0 patients) | 0% (0 patients) |
| B3 | 0% (0 patients) | 0% (0 patients) | 0% (0 patients) |
| Severity in wPCDAI | 65 (35 to 77.5) | 65 (22.5 to 87.5) | 26.3 (15 to 57.5) |

Medications

| | | | |
|---|---|---|---|
| Mesalamine | 0% (0 patient) | 20% (1 patients) | 50% (2 patients) |
| Azathioprine | 20% (1 patient) | 20% (1 patient) | 25% (1 patient) |
| Methotrexate | 80% (4 patient) | 60% (3 patient) | 0% (0 patient) |
| Anti - TNF | 100% (5 patients) | 100% (5 patients) | 0% (0 patient) |
| Nutritional therapy | 20% (1 patient) | 20% (1 patient) | 25% (1 patient) |
| Steroids | 60% (3 patients) | 60% (3 patients) | 50% (2 patients) |

Median time since diagnosis

| | | | |
|---|---|---|---|
| Median | 31.7 (29.6 to 37.2) | 36.3 (29.6 to 37.2) | 32 (24.2 to 33.7) |

* indicates significance detected between

TABLE 7
Flow cytometry panels

| | | | PREDICT | PREDICT | PREDICT |
|---|---|---|---|---|---|
| | | | Panel #1.1 | Panel #1.2 | Panel #1.3 |
| | | | T and non-T | T cells; basic non-T | T cells; basic non-T |
| Laser | Filter | Fluorophore | population | subpopulations | subpopulations |
| UV | 379/28 | BUV 395 | CD11b | CD8a | CD8a |
| | 450/50 | BUV 496 | CD8a | | |
| | 670/25 | BUV 661 | CD11c | CD11c | CD11c |
| | 730/45 | BUV 737 | CD14 | CD14 | CD14 |
| Violet | 450/50 | BV421/V450/PacificBlue | CCR7 | CCR7 | CCR7 |
| | 510/10 | BV510, Aqua | Live/Dead Aqua | Live/Dead Aqua | Live/Dead Aqua |
| | 610/20 | BV605 | CD45RA | CD45RA | CD45RA |
| | 712/21 | BV711 | CD123 | CD123 | CD123 |
| | 795/20 | BV786 | CD4 | CD4 | CD4 |
| Blue | 530/30 | FITC/AlexaFluor488 | CD8b | CD56 | CD56 |
| | 695/40 | PerCP, PerCP-Cy5.5 | CD45 | CD45 | CD45 |
| Yellow | 586/15 | PE | CD3 | CD3 | CD3 |
| Green | 610/20 | PE-Texas Red/PE-CF- | CD56 | | |
| | | 594/ECD | | | |
| | 780/60 | PE-Cy7 | TCRgd | CD20 | CD20 |
| Red | 670/14 | APC/AlexaFluor647/eFluor660 | EpCAM | CD11b | CD138 |
| | 730/45 | AlexaFluor700 | CD20 | | |
| | 780/60 | eFlour 780/APC-Cy7/APC-H7 | HLA-DR | HLA-DR | HLA-DR |
| # samples: | 10 | | | | |

| | | | | PREDICT | PREDICT |
|---|---|---|---|---|---|
| | | | | Panel #2.2 | Panel #2.3 |
| | | | PREDICT | ILC; unconventional | ILC; unconventional |
| Laser | Filter | Fluorophore | Panel #2.1 | T cells | T cells |
| UV | 379/28 | BUV 395 | | CD8a | CD8a |
| | 450/50 | BUV 496 | | | |
| | 670/25 | BUV 661 | | CD3 | CD3 |
| | 730/45 | BUV 737 | | CD127 - biotin | CD127 - biotin |
| Violet | 450/50 | BV421/V450/PacificBlue | | CD117 | CD117 |
| | 510/10 | BV510, Aqua | | Live/Dead Aqua | Live/Dead Aqua |
| | 610/20 | BV605 | | CRTH2 | CRTH2 |
| | 712/21 | BV711 | | CD161 | CD161 |
| | 795/20 | BV786 | | CD4 | CD4 |
| Blue | 530/30 | FITC/AlexaFluor488 | | CD8b | CD8b |
| | 695/40 | PerCP, PerCP-Cy5.5 | | CD45 | CD45 |
| Yellow | 586/15 | PE | | NKp44 | alpha4beta7 |
| Green | 610/20 | PE-Texas Red/PE-CF- | | | |
| | | 594/ECD | | | |
| | 780/60 | PE-Cy7 | | TCRgd | TCRgd |
| Red | 670/14 | APC/AlexaFluor647/eFluor660 | | EpCAM | EpCAM |
| | 730/45 | AlexaFluor700 | | | |
| | 780/60 | eFlour 780/APC-Cy7/APC-H7 | | Lineage | Lineage |
| | | | | (CD14/Cd20/CD11c/ | (CD14/Cd20/CD11c/ |
| | | | | CD11b/CD56) | CD11b/CD56) |

TABLE 8
FGID end cell cluster descriptive names and short curated names. Table is organized by cluster name, avg_rnkscr, dataset, cell type, short name; cluster name, avg_rnkscr, dataset, cell type, short name; etc.

Hs.Bclls.Bclls_cycling.GerminalCenter_HLA-DQB1_CD40_CD79B.DarkZone_CXCR4_AICDA_Kappa, 2560.562812, FG, Bclls, FG.B/DZ.AICDA.IGKC;
Hs.Bclls.Bclls_cycling. GerminalCenter_HLA-DQB1_CD40_CD79B.DarkZone_CXCR4_AICDA_Lambda, 2267.431256, FG, Bclls, FG.B/DZ.AICDA.IGLC2;
Hs.Bclls.Bclls_cycling.GerminalCenter_HLA-DQB1_CD40_CD79B.GerminalCenter_Ribosomal, 62.79408799, FG, Bclls, FG.B/GC.ACTB.TMSB4X;
Hs.Bclls.Bclls_cycling.GerminalCenter_HLA-DQB1_CD40_CD79B.LightZone_HLA_CD83, 3425.913916, FG, Bclls, FG.B/LZ.CD74.CD83;
Hs.Bclls.Bclls_cycling.GerminalCenter_HLA-DQB1_CD40_CD79B.p034_dominated, 89.95804914, FG Bclls, FG.B.BASP1.ACTG1;
Hs.Bclls.Bclls_cycling.proliferating_DarkZone, 27095.18304, FG, Bclls, FG.B/DZ.TOP2A.UBE2C; Hs.Bclls.Bclls_cycling.tcll_doublets, 19377.05009, FG, Doublets/Bclls,
FG.Doublets/B.CD3D.CD2; Hs.Bclls.Bclls_noncycling.CD27_memory, 1147.616245, FG, Bclls, FG.B.TNFRSF13B.GPR183;
Hs.Bclls.Bclls_noncycling.FCER2_IGHD_follicular_transitioning, 1305.111499, FG, Bclls, FG.B.IGHD.FCER2; Hs.Bclls.Bclls_noncycling.IGLC3_IGLC6_IGLC7, 87293.93474, FG, Bclls, FG.B.IGLC6.IGLC7;
Hs.Bclls.Bclls_noncycling.p035_dominated, 279.5234282, FG, Bclls, FG.B.CXCR4.PABPC1; Hs.Bclls.Bclls_noncycling.p050_dominated_SPILLOVER,
6273.964637, FG, Doublets/Bclls, FG.Doublets/B.TPSB2.DEFA5; Hs.Endth.activated_endothelium, 216.3929873, FG, Endth, FG.Endth.FOS.FOSB;
Hs.Endth.arterioles_FRZB_NOTCH3, 16154.73509, FG, Endth, FG.Endth/Art.FRZB.NOTCH3; Hs.Endth.capillaries_PLVAP_FLT1, 149.8849781, FG, Endth,
FG.Endth/Cap.PLVAP.FLT1; Hs.Endth.doublets_epithelial, 404.2747987, FG, Doublets/Endth, FG.Doublets/Endth.RBP2.PIGR; Hs.Endth.doublets_plasma, 233.1517527,
FG, Doublets/Endth, FG.Doublets/Endth.IGKC.JCHAIN; Hs.Endth.doublets_tcll, 2778.001695, FG, Doublets/Endth, FG.Doublets/Endth.LTB.CD52;
Hs.Endth.lymphatics_CCL21_LYVE1_PROX1.IGF2, 1359.540037, FG, Endth, FG.Endth/Lymph.TFPI.LYVE1; Hs.Endth.lymphatics_CCL21_LYVE1_PROX1.mitochondrial,
1607.450574, FG, Endth, FG.Endth/Lymph.CCL21.PROX1; Hs.Endth.lymphatics_CCL21_LYVE1_PROX1.MYADM_DDX34, 3982.428739, FG, Endth, FG.Endth/Lymph.RP11-
136K72.LYVE1; Hs.Endth.lymphatics_CCL21_LYVE1_PROX1.ribosomal, 13596.30854, FG, Endth, FG.Endth/Lymph.CCL21.LYVE1;
Hs.Endth.postcapillaryvenules_ACKR1_MADCAM1, 12931.62061, FG, Endth, FG.Endth/Ven.ACKR1.MADCAM1; Hs.Epith.absorbative_p44dominated.APOA_late_entero,
3709.078173, FG, Epith, FG.EC.APOC3.APOA1; Hs.Epith.absorbative_p44dominated.glutathione_early_entero_GSTA, 2839.552039, FG, Epith, FG.EC.GSTA2.AADAC;
Hs.Epith.absorbative_p44dominated.LIPH_ELF3_SQSTM1_entero, 537.4365036, FG, Epith, FG.EC.RBP2.ALDOB; Hs.Epith.crypt_OLFM4_AGR2.ALDOB_enterocyte,
139239.2843, FG, Epith, FG.EC.FABP6.CUBN; Hs.Epith.crypt_OLFM4_AGR2.crypt_based_entero.early_absorbtive, 1364.260322, FG, Epith, FG.EC.C15orf48.OLFM4;
Hs.Epith.crypt_OLFM4_AGR2.crypt_based_entero.early_absorbtive_TPT1_CCL25, 48.76087015, FG, Epith, FG.EC.RPL4.REG1A;
Hs.Epith.crypt_OLFM4_AGR2.crypt_based_entero.mitochondrial, 374.7902862, FG, LowQual/Epith, FG.EC.XIST.OLFM4;
Hs.Epith.crypt_OLFM4_AGR2.crypt_based_entero.Tuft, 6.54E+301, FG, Epith, FG.Tuft.GNG13.SH2D6; Hs.Epith.crypt_OLFM4_AGR2.doublets_tcll, 32923.90378, FG,
Doublets/Epith, FG.Doublets/EC.CD37.CD52; Hs.Epith.crypt_OLFM4_AGR2.early_mid_entero_p044_dominated.AGR2_CCL25, 540.113307, FG, LowQual/Epith,
FG.EC.OLFM4.LDHB; Hs.Epith.crypt_OLFM4_AGR2.early_mid_entero_p044_dominated.ANXA10_VSIG2_LYZ, 30005.05995, FG, LowQual/Epith, FG.EC.ANXA10.VSIG2;
Hs.Epith.crypt_OLFM4_AGR2.early_mid_entero_p044_dominated.B3GNT7, 267.2863928, FG, LowQual/Epith, FG.EC.DMBT1.NPM1;
Hs.Epith.crypt_OLFM4_AGR2.early_mid_entero_p044_dominated.BEST4_CFTR_SPIB, 6.35E+301, FG LowQual/Epith, FG.EC.NPY.ADGRF1;
Hs.Epith.crypt_OLFM4_AGR2.early_mid_entero_p044_dominated.CTGF_ATF3, 573.9455072, FG, LowQual/Epith, FG.EC.OLFM4.MARCKSL1;
Hs.Epith.crypt_OLFM4_AGR2.early_mid_entero_p044_dominated.FABP1, 213.189666, FG, LowQual/Epith, FG.EC.DMBT1.MT1H;
Hs.Epith.crypt_OLFM4_AGR2.early_mid_entero_p044_dominated.GSTA1_CBR1, 369.6828905, FG LowQual/Epith, FG.EC.DMBT1.ADH1C;
Hs.Epith.crypt_OLFM4_AGR2.early_mid_entero_p044_dominated.mitochondrial, 282.472776, FG LowQual/Epith, FG.EC.DMBT1.REG1A;
Hs.Epith.crypt_OLFM4_AGR2.early_mid_entero_p044_dominated.OLFM4_SOX4, 561.6041083, FG, LowQual/Epith, FG.EC.OLFM4.SLC12A2;
Hs.Epith.crypt_OLFM4_AGR2.early_mid_entero_p044_dominated.Proliferating, 872.8060287, FG, LowQual/Epith, FG.EC.CENPF.PTTG1;
Hs.Epith.crypt_OLFM4_AGR2.early_mid_entero_p044_dominated.TK1_DHFR_H2AZ, 6243.34292, FG, LowQual/Epith, FG.EC.KIAA0101.FAM111B;
Hs.Epith.crypt_OLFM4_AGR2.enteroendocrine, 1.38E+302, FG, Epith, FG.Enteroendocrine.SCG3.ISL1; Hs.Epith.crypt_OLFM4_AGR2.goblet.CLCA1_ZG16_villi_based,
47449.92585, FG, Epith, FG.Goblet.ITLN1.ZG16; Hs.Epith.crypt_OLFM4_AGR2.goblet.crypt_based, 38495.28732, FG, Epith, FG.Goblet.SPINK4.ITLN1;
Hs.Epith.crypt_OLFM4_AGR2.goblet.mitochondrial, 32681.74132, FG, Epith, FG.Goblet.FCGBP.SPINK4; Hs.Epith.crypt_OLFM4_AGR2.goblet.REG4_SPINK4_TFF3,
53684.97798, FG, Epith, FG.Goblet.ITLN1.SPINK4; Hs.Epith.crypt_OLFM4_AGR2.mid_entero_p044_dominated, 1371.218357, FG, LowQual/Epith, FG.EC.DMBT1.ADH1C;
Hs.Epith.crypt_OLFM4_AGR2.mito, 481.9223398, FG, LowQual/Epith, FG.EC.MT-CO2.MT-CO3; Hs.Epith.crypt_OLFM4_AGR2.paneth, 102826.2325, FG, Epith,
FG.Paneth.ITLN2.PRSS2; Hs.Epith.crypt_OLFM4_AGR2.proliferating, 30391.16577, FG, Epith, FG.Prolif/EC.UBE2C.TOP2A;
Hs.Epith.crypt_OLFM4_AGR2.StemCell.APOA_p044_domainted, 7808.862639, FG, LowQual/Epith, FG.EpithStem.SMOC2.RGMB;
Hs.Epith.crypt_OLFM4_AGR2.StemCell.HLA_CCL15_CCL25, 3809.629873, FG, Epith, FG.EC.CCL15.SPIB; Hs.Epith.crypt_OLFM4_AGR2.StemCell.LGR5_OLFM4, 10844.93867,
FG, Epith, FG.EpithStem.LGR5.SMOC2; Hs.Epith.goblet, 2.69E+302, FG, Epith, FG.Goblet.TFF2.MUC6; Hs.Fibro.ADAMDEC1_FBLN1_SEPP1_ASPN, 221.2385525, FG, Fibro,
FG.Fibro.ADAMDEC1.FBLN1; Hs.Fibro.ADAMDEC1_LUM_CFH_IFITM1, 54.92186782, FG, Fibro, FG.Fibro.IFITM1.ADAMDEC1;
Hs.Fibro.APOE_CCL8_CCL11.ACTA2_CCL8_PTCH1, 107.5428813, FG, Fibro, FG.Fibro.RPS10.RPS12; Hs.Fibro.APOE_CCL8_CCL11.COL3A1, 3.305424445, FG, Fibro,
FG.Fibro.MT-ND4L; Hs.Fibro.APOE_CCL8_CCL11.schwann_PMP22_MFAP4_LMNA_ACKR3, 367.9638187, FG, Fibro, FG.Fibro.ACKR3.RCN3;
Hs.Fibro.CCL13_CCL11_LUM_IGF1, 2649.996359, FG, Fibro, FG.Fibro.CCL13.CFD; Hs.Fibro.CFD_PTN_PLTP, 227.3961904, FG, Fibro, FG.Fibro.CFD.IGHM;
Hs.Fibro.CPM_EDRNB_F3_POSTN_hirschsprung, 30123.09047, FG, Fibro, FG.Fibro.FIGF.EDNRB; Hs.Fibro.CXCL14_ADAMDEC1, 437.9978875, FG, Doublets/Fibro,
FG.Doublets/Fibro.IGKC.JCHAIN; Hs.Fibro.doublet_bcll, 43407.07235, FG, Doublets/Fibro, FG.Doublets/Fibro.VPREB3.MS4A1; Hs.Fibro.doublet_epithelial, 39225.93063,
FG, Doublets/Fibro, FG.Doublets/Fibro.RBP2.APOA1; Hs.Fibro.doublet_mast, 55918.12359, FG, Doublets/Fibro, FG.Doublets/Fibro.HPGDS.CPA3; Hs.Fibro.doublet_mito,
357.1345338, FG, Doublets/Fibro, FG.Doublets/Fibro.MT-CO3.MT-ND3; Hs.Fibro.doublet_tcll, 10324.14495, FG, Doublets/Fibro, FG.Doublets/Fibro.CCL5.KLRB1;
Hs.Fibro.FBLN1_CCL11_CFD_FABP5_TNC, 651.0942481, FG, Fibro, FG.Fibro.CCL11.FABP5; Hs.Fibro.myofibroblast_MYH11_ACTA2_ACTG2.CFD_FBLN1_CCL11,
290.0325714, FG, Fibro, FG.Fibro.ACTA2.TAGLN; Hs.Fibro.myofibroblast_MYH11_ACTA2_ACTG2.CXCL14_ADAMDEC1_ABCA6, 242.9787487, FG, Fibro,
FG.Fibro.ACTA2.SOSTDC1; Hs.Fibro.myofibroblast_MYH11_ACTA2_ACTG2.myofibroblast_ACTG2_MYH11, 75985.62763, FG, Fibro, FG.MyoFibro.DES.MYH11;
Hs.Fibro.tcllzonestromal_CCL19_CCL21_FDCSP.ADAMDEC1_CFD_CCL11_APOE, 1988.442935, FG, Fibro, FG.Fibro.CCL19.C3;
Hs.Fibro.tcllzonestromal_CCL19_CCL21_FDCSP.C3_mito, 5098.736724, FG, Fibro, FG.Fibro.C3.FDCSP;
Hs.Fibro.tcllzonestromal_CCL19_CCL21_FDCSP.tcllzonestromal_CCL19_CCL21, 69709.46977, FG, Fibro, FG.Fibro.CCL21.CCL19;
Hs.Fibro.tcllzonestromal_CCL19_CCL21_FDCSP.TNFSF11_LGALS1_CD9, 21748.45581, FG, Fibro, FG.Fibro.TNFSF11.CD24; Hs.Glial.activated_CYR61, 309.1112751, FG,
Glial, FG.Glial.IER2.JUNB; Hs.Glial.BCYRN1_GPR155_RHOB_VWA1, 146.7588488, FG, Glial, FG.Glial.BCYRN1.SPARC; Hs.Glial.CD9_SPARC_CRYAB, 37.69354389, FG, Glial,
FG.Glial.S100A10.IGHA2; Hs.Glial.CYR61_SPARC, 1059.630551, FG, Glial, FG.Glial.HSPA1B.SOCS3; Hs.Glial.doublets_plasma_myeloid, 270.6790591, FG, Doublets/Glial,
FG.Doublets/Glial.IGKC.JCHAIN; Hs.Glial.doublets_tcll, 5153.112278, FG, Doublets/Glial, FG.Doublets/Glial.CD2.CD3E;
Hs.Glial.folliculardendritic_FDCSP_CR2_SPIB_CXCL13, 4.77E+301, FG, Glial, FG.Glial/fDC.FDCSP.CXCL13; Hs.Glial.IGFBP7_SCN7A_HAND2, 19.21776326, FG, Glial,
FG.Glial.HAND2.MT-ND4L; Hs.Glial.mitochondrial, 179.6379589, FG, Glial, FG.Glial.MS4A6A.MT-CO2; Hs.IGA_IGK_Plsma.doublets.doublets_epithelial, 13098.05344, FG,
Doublets/Plsma, FG.Doublets/IGA.REG1A.LUM; Hs.IGA_IGK_Plsma.doublets.doublets_mast, 65177.88163, FG, Doublets/Plsma, FG.Doublets/IGA.TPSAB1.TPSB2;
Hs.IGA_IGK_Plsma.doublets.doublets_myeloid, 37074.09008, FG, Doublets/Plsma, FG.Doublets/IGA.AIF1.TYROBP; Hs.IGA_IGK_Plsma.doublets.doublets_stromal,
39543.92313, FG, Doublets/Plsma, FG.Doublets/IGA.CCL11.IGFBP7; Hs.IGA_IGK_Plsma.Plsma_1.Bclls, 1905.2326, FG, Plsma, FG.Plsma/IgA.HLA-DRA.MS4A1;
Hs.IGA_IGK_Plsma.Plsma_1.CD79A, 53.8403667, FG, Plsma, FG.Plsma/IgA.IGKC.DUSP5; Hs.IGA_IGK_Plsma.Plsma_1.doublet_tcll, 6204.663693, FG, Doublets/Plsma,
FG.Doublets/IGA.KLRB1.CD3D; Hs.IGA_IGK_Plsma.Plsma_1.IGG, 1683.460548, FG, Plsma, FG.Plsma/IgA.IGHG4.IGHG3; Hs.IGA_IGK_Plsma.Plsma_1.IGKV_IGLV_unique,
5362.934483, FG, Plsma, FG.Plsma/IgA.IGKV1-12.IGKV3-20; Hs.IGA_IGK_Plsma.Plsma_1.IGKV_unique, 1559.275561, FG, Plsma, FG.Plsma/IgA.IGKV3-20.IGKV4-1;
Hs.IGA_IGK_Plsma.Plsma_1.mito_high, 238.7033947, FG, Plsma, FG.Plsma/IgA.MT-ATP6.MT-ND1; Hs.IGA_IGK_Plsma.Plsma_1.Plsma, 220.8729699, FG, Plsma,
FG.Plsma/IgA.IGKC.AL9287683; Hs.IGA_IGK_Plsma.Plsma_1.spill_epi, 174.4181759, FG, Plsma, FG.Plsma/IgA.IGHA1.AL9287683; Hs.IGA_IGK_Plsma.Plsma_2.activated,
167.6665832, FG, Plsma, FG.Plsma/IgA.IGHA2.HSPA1B; Hs.IGA_IGK_Plsma.Plsma_2.doublet_stromal, 228.9033408, FG, Doublets/Plsma, FG.Doublets/IGA.HSPA1B.LMNA;
Hs.IGA_IGK_Plsma.Plsma_2.IGKV_unique, 656.7218767, FG, Plsma, FG.Plsma/IgA.IGKV4-1.IGKV1-5; Hs.IGA_IGK_Plsma.Plsma_2.JCHAIN, 173.3224284, FG, Plsma,
FG.Plsma/IgA.BIRC3.IGKV1D-8; Hs.IGA_IGK_Plsma.Plsma_2.ribosomal, 96.93748758, FG, Plsma, FG.Plsma/IgA.IGHA2.IGKC; Hs.IGA_IGK_Plsma.Plsma_2.spill_epi,
1768.360462, FG, Plsma, FG.Plsma/IgA.IGHA2.APOA1; Hs.IGA_IGL_Plsma.doublets_stromal, 25956.14572, FG, Doublets/Plsma, FG.Doublets/IGA.CALD1.IGFBP7;
Hs.IGA_IGL_Plsma.doublets_Tcll, 3080.979371, FG, Doublets/Plsma, FG.Doublets/IGA.KLRB1.IL32; Hs.IGA_IGL_Plsma.IGLC2_IGLC3, 3501.294965, FG, Plsma,
FG.Plsma/IgA.IGLC3.IGLC2; Hs.IGA_IGL_Plsma.IGLL5_JCHAIN, 8087.038016, FG, Plsma, FG.Plsma/IgA.IGLL5.IGLV2-14; Hs.IGG_Plsma_Bcll.Bcll_like.Bclls, 7382.91358, FG,
Plsma, FG.Plsma/IgG.HLA-DQA1.MS4A1; Hs.IGG_Plsma_Bcll.Bcll_like.mature_IGHG1.Bcll_CD37_CD20, 1709.302096, FG, Plsma, FG.Plsma/IgG.TCL1A.IGHG3;
Hs.IGG_Plsma_Bcll.Bcll_like.mature_IGHG1.CD2_Tclls_doublet, 1768.159436, FG, Doublets/Plsma, FG.Doublets/IgG.IGHGP.IGHG3;
Hs.IGG_Plsma_Bcll.Bcll_like.mature_IGHG1.IGKC, 3302.403862, FG, Plsma, FG.Plsma/IgG.IGHGP.IGHG2;
Hs.IGG_Plsma_Bcll.Bcll_like.mature_IGHG1.Marginal_PPIB_ISG20_MZB1_transitioning_Bclls, 1368.371383, FG, Plsma, FG.Plsma/IgG.HLA-DRB5.IGHG2;
Hs.IGG_Plsma_Bcll.Bcll_like.Tclls_doublets, 2350.862018, FG, Doublets/Plsma, FG.Doublets/Plsma.CXCR4.CD3D; Hs.IGG_Plsma_Bcll.IGKC, 22845.05117, FG, Plsma,
FG.Plsma/IgG.IGHGP.IGHG2; Hs.IGG_Plsma_Bcll.IGLC1_IGLC2, 16984.52194, FG, Plsma, FG.Plsma/IgG.IGHGP.IGHG2; Hs.IGM_Plsma, 5854.735788, FG, Plsma,
FG.Plsma/IgM.IGHM.IGLV2-14; Hs.Mloid.dendritic_CLEC9A_BATF3_IDO1, 21892.35356, FG, Mloid, FG.cDC1.CLEC9A.XCR1;
Hs.Mloid.dendritic_CLECL10A_CD1C_CD207.C1Q_VSIG4, 255.0030762, FG, Mloid, FG.Mono/Mac/cDC2.LYZ.CLEC10A;
Hs.Mloid.dendritic_CLECL10A_CD1C_CD207.CSF2RA_LTB_RUNX3.HLADQ_ribo, 132.2019958, FG, Mloid, FG.cDC2.AREG.CD1C;
Hs.Mloid.dendritic_CLECL10A_CD1C_CD207.CSF2RA_LTB_RUNX3.IL22RA2_BTLA_LTB, 2001.285167, FG, Mloid, FG.DC.LTB.IL22RA2;
Hs.Mloid.dendritic_CLECL10A_CD1C_CD207.CSF2RA_LTB_RUNX3.LILRA2, 62.61518627, FG, Mloid, FG.cDC2.AREG.PLAC8;
Hs.Mloid.dendritic_CLECL10A_CD1C_CD207.CSF2RA_LTB_RUNX3.TMEM176_immature, 348.5925006, FG, Mloid, FG.cDC2.CLEC10A.CD207;
Hs. Mloid.dendritic_CLECL10A_CD1C_CD207.doublets_enterocyte, 83486.82761, FG, Mloid, FG.DC.LTB.GSN; Hs.Mloid.dendritic_CLECL10A_CD1C_CD207.doublets_plasma,
1715.624546, FG, Mloid, FG.Mono/cDC2.FCER1A.CD1C; Hs.Mloid.dendritic_CLECL10A_CD1C_CD207.HLADQ_HLADR_CD207, 1545.211455, FG, Mloid,
FG.Mono/cDC2.AREG.IL1R2; Hs.Mloid.dendritic_CLECL10A_CD1C_CD207.migratory_CD207_FSCN1, 419.0893564, FG, Mloid, FG.cDC2.CD207.FGR;
Hs.Mloid.dendritic_CLECL10A_CD1C_CD207.S100A8_S100A9, 58255.54511, FG, Mloid, FG.Mono/cDC2.FCN1.S100A9; Hs.Mloid.dendritic_migratory_CCR7_FSCN1,
39966.32774, FG, Mloid, FG.DC.CCR7.FSCN1; Hs.Mloid.doublets_lymphoid.CQ1A_CQ1C_macrophage, 1634.385947, FG, Doublets/Mloid,
FG.Doublets/Mono/Mac.C1QA.CD3D; Hs.Mloid.doublets_lymphoid.doublets_lymphocytes, 2372.39601, FG, Doublets/Mloid, FG.Doublets/Mloid.TRAC.TRBC2;
Hs.Mloid.doublets_lymphoid.FCER1A_CD1E_dendritic, 327.4482583, FG, Doublets/Mloid, FG.Doublets/Mloid.CD3D.KLRB1; Hs.Mloid.doublets_lymphoid.IL32_KLDR1,
2977.80043, FG, Doublets/Mloid, FG.Doublets/Mloid.TRBC2.CD3E; Hs.Mloid.doublets_lymphoid.macrophage, 2295.755258, FG, Doublets/Mloid,
FG.Doublets/Mloid.TRAC.RORA; Hs.Mloid.doublets_lymphoid.plasmacytoid_dendritic, 25725.33077, FG, Mloid, FG.pDC.IRF7.LILRA4;
Hs.Mloid.doublets_lymphoid.Ribo_p009dominated, 915.9959289, FG, Doublets/Mloid, FG.Doublets/Mloid.CD3D.TRAC;
Hs.Mloid.doublets_mito_dendritic.dendritic_CLEC10A_CD207, 31.22256823, FG, Mloid, FG.cDC2.RPL23A.RPS29;
Hs.Mloid.doublets_mito_dendritic.dendritic_CLEC10A_IL1B, 7.747069603, FG, Mloid, FG.cDC2.RPL27.RPS12; Hs.Mloid.doublets_mito_dendritic.dendritic_CLEC9A_IRF8,
121.2020705, FG, Mloid, FG.cDC1.SNX3.ID01; Hs.Mloid.doublets_mito_dendritic.doublets, 504.6820828, FG, Doublets/Mloid, FG.Doublets/cDC2.IGHA1.IGKC;
Hs.Mloid.doublets_mito_dendritic.mito_high, 6.49408767, FG, Mloid, FG.cDC2.RPL37.RPS12; Hs.Mloid.doublets_plasma_macrophage, 1044.423265, FG, Doublets/Mloid,
FG.Doublets/Mac.MT-CO3-MT-CO1; Hs.Mloid.doublets_stromal, 79777.54355, FG, Doublets/Mloid, FG.Doublets/Mac.ADAMDEC1.COL3A1;
Hs.Mloid.macrophage_C1Q_CD63_CD14_AXL, 1148.392519, FG, Mloid, FG.Mac.C1QB.SEPP1; Hs.Mloid.macrophage_chemokine.doublets, 2075.625389, FG, Mloid,
FG.Mac.CCL3.HES1; Hs.Mloid.macrophage_chemokine.HLADQ_IFITM3_CXCL3_F13A1, 3750.159334, FG, Mloid, FG.Mac.CXCL3.CXCL8;
Hs.Mloid.macrophage_chemokine.LYZ, 784.2273149, FG, Mloid, FG.Mac.CXCL8.IL1B; Hs.Mloid.macrophage_tissue_MMP_APOE_CD68, 29037.98061, FG, Mloid,
FG.Mac.APOE.PTGDS; Hs.Mloid.proliferating, 49421.55322, FG, Mloid, FG.Mac.MKI67.LYZ; Hs.Mstcl.doublets_plasma, 0, FG, Doublets/Mstcl,
FG.Doublets/Mstcl.JCHAIN.IGKC; Hs.Mstcl.doublets_tcll, 0, FG, Doublets/Mstcl, FG.Doublets/Mstcl.CD3D.LTB; Hs.Mstcl.doublets_epithelial, 0, FG, Doublets/Mstcl,
FG.Doublets/Mstcl.DEFA5.REG1A; Hs.Mstcl.Mstcl, 103676.0868, FG, Mstcl, FG.Mstcl.HPGDS.TPSAB1; Hs.Tclls.activated.activated_cytotoxic, 10216.49502, FG, Tclls,
FG.T/NK.CCL4.GZMB; Hs.Tclls.activated.activated_p035_dominated, 1319.136173, FG, Tclls, FG.Tclls.JUN.FOS; Hs.Tclls.activated.cytotoxic_CD3.activated, 3927.535424,
FG, Tclls, FG.T.GZMK.IFNG; Hs.Tclls.activated.cytotoxic_CD3.CCR7_IL7R_SELL_KLF2_naive, 3833.453314, FG, Tclls, FG.T.GZMK.CMC1;
Hs.Tclls.activated.cytotoxic_CD3.CD8A_CD8B_CCL5, 5546.152101, FG, Tclls, FG.T.GZMK.CRTAM; Hs.Tclls.activated.cytotoxic_CD3.IL10_MAF, 4936.187049, FG, Tclls,
FG.T.GZMK.GZMA; Hs.Tclls.activated.cytotoxic_CD3.KLRB1_HOPX_RORA, 2276.96671, FG, Tclls, FG.T.GZMK.NKG7;
Hs.Tclls.activated.cytotoxic_CD3.LGALS3_S100A10_Mphage_doublets, 6637.054014, FG, Doublets/Tclls, FG.Doublets/T.CAV1.HAVCR2;
Hs.Tclls.activated.cytotoxic_CD3.MS4A1_CD79A_Bclls_doublets, 24234.66643, FG, Doublets/Tclls, FG.Doublets/T.VPREB3.MEF2C;
Hs.Tclls.activated.cytotoxic_CD3.Plsma_Mstcl_doublets, 4648.224401, FG, Doublets/Tclls, FG.Doublets/T.GZMK.GZMA; Hs.Tclls.activated.doublets_p017_dominated,
1828.467739, FG, Doublets/Tclls, FG.Doublets/T.IGLC2.JCHAIN; Hs.Tclls.activated.IL7R.CCR7_FOS_JUN_activated, 1577.332078, FG, Tclls, FG.T.CD69.JUN;
Hs.Tclls.activated.IL7R.CD8A_CD8B_RUNX3, 1223.178345, FG, Tclls, FG.T.CCL5.CD8B; Hs.Tclls.activated.IL7R.doublets_p043_dominated, 1875.163521, FG, Doublets/Tclls,
FG.Doublets/T.DEFA5.TXNIP; Hs.Tclls.activated.IL7R.GZMK_NCR3, 2649.011981, FG, Tclls, FG.T.TRDC.SOX4; Hs.Tclls.activated.IL7R.ICOS_CCR6, 2508.377312, FG, Tclls,
FG.T.RORA.CCR6; Hs.Tclls.activated.IL7R.IFNGR_ISG_p037_dominated, 733.93422, FG, LowQual/Tclls, FG.T.MT-ND4L.TSC22D3;
Hs.Tclls.activated.IL7R.spillover_doublets_p044_dominated, 34541.49706, FG, Doublets/Tclls, FG.Doublets/T.RBP2.APOA4;
Hs.Tclls.activated.ribosomal_p049_dominated.DEFA5_DEFA6_p050_dominated, 1109.48943, FG, Doublets/Tclls, FG.Doublets/T.DEFA5.AC0925804;
Hs.Tclls.activated.ribosomal_p049_dominated.IGHA_Bclls_doublet.CD4_KLRB1_CD40LG_LTB, 1338.945231, FG, Doublets/Tclls, FG.Doublets/T.IGHM.IGHA2;
Hs.Tclls.activated.ribosomal_p049_dominated.IGHA_Bclls_doublet.CD8A_CD8B_CD160, 1625.032084, FG, Doublets/Tclls, FG.Doublets/T.CCL5.IGHM;
Hs.Tclls.activated.ribosomal_p049_dominated.IGHA_Bclls_doublet.FOXP3_CTLA4_TIGIT, 339.0907098, FG, LowQual/Tclls, FG.T.IL32.CARD16;
Hs.Tclls.activated.ribosomal_p049_dominated.TRDC_TRGC2_CD7_SOX4, 7334.82634, FG, LowQual/Tclls, FG.T.TRDC.ZNF683; Hs.Tclls.cytotoxic, 40674.5418, FG, Tclls,
FG.T/NK/ILC.GNLY.TYROBP; Hs.Tclls.resting_naive.KLRB1_TIGIT_MAF, 191.8988095, FG, Tclls, FG.T/NK/ILC.MAF.RPS26; Hs.Tclls.resting_naive.SELL_SOX4_ribo,
1748.727688, FG, Tclls, FG.T/NK/ILC.CCR7.SELL; Hs.Tclls.resting_naive.spillover_p050_dominated, 512.4329663, FG, LowQual/Tclls, FG.T.CCR7.SELL;

TABLE 9
CD end cell cluster descriptive names and short curated names. Table is organized by cluster name, avg_rnkscr, dataset, cell type, short name; cluster name, avg_rnkscr, dataset, cell type, short name; etc.

CD.Bclls.CD69_GPR183_IGHG1, 814.7361818, CD, Bclls, CD.B.CD69.GPR183; CD.Bclls.doublets_epithelial, 61833.71294, CD, Doublets/Bclls, CD.Doublets/B.APOA1.APOA4;
CD.Bclls.doublets_epithelial.CD79A_IGHD, 1803.351831, CD, Doublets/Bclls, CD.Doublets/B.DEFA5.DEFA6; CD.Bclls.doublets_epithelial.IGKC_IGHM_GPR183,
1493.143925, CD, Doublets/Bclls, CD.Doublets/B.DEFA5.DEFA6; CD.Bclls.doublets_epithelial.IGLC2, 1218.379242, CD, Doublets/Bclls, CD.Doublets/B.DEFA5.IGLC2;
CD.Bclls.doublets_epithelial.KLF6_KLF2_JUN, 1455.742221, CD, Doublets/Bclls, CD.Doublets/B.DEFA5.AL9287683; CD.Bclls.doublets_epithelial.macrophage,
1850.734457, CD, Doublets/Bclls, CD.Doublets/B.C1QB.C1QA; CD.Bclls.doublets_epithelial.SMIM14, 1242.273582, CD, Doublets/Bclls, CD.Doublets/B.DEFA5.DEFA6;
CD.Bclls.doublets_epithelial.ZFP36, 185.7611501, CD, Doublets/Bclls, CD.Doublets/B.DEFA5.REG1A; CD.Bclls.doublets_Tclls.doublets_epithelial, 5466.846911, CD,
Doublets/Bclls, CD.Doublets/B.KLRB1.IL7R; CD.Bclls.doublets_Tclls.doublets_Tclls, 5508.464141, CD, Doublets/Bclls, CD.Doublets/B.IL32.KLRB1;
CD.Bclls.doublets_Tclls.IFI44L_IFI6, 3673.328601, CD, Doublets/Bclls, CD.Doublets/B.CD3D.CD2; CD.Bclls.doublets_Tclls.IGHG4, 6561.267321, CD, Doublets/Bclls,
CD.Doublets/B.CD3D.CD7; CD.Bclls.doublets_Tclls.IL7R_IGHA1_Doublet, 10806.38244, CD, Doublets/Bclls, CD.Doublets/B.IL7R.KLRB1; CD.Bclls.doublets_Tclls.ribosomal,
8233.049085, CD, Doublets/Bclls, CD.Doublets/B.CD3G.GIMAP7; CD.Bclls.doublets_Tclls.Tclls_CXCR6_IL2RA, 48326.7597, CD, Doublets/Bclls,
CD.Doublets/B.IL1R2.CXCR6; CD.Bclls.GerminalCenter_CD27_AICDA_CD38, 7503.379832, CD, Bclls, CD.B/DZ.RGS13.AICDA;
CD.Bclls.GerminalCenter_MKI67_AICDA_ITGAE, 57896.35134, CD, Bclls, CD.B/DZ.AICDA.PLK1; CD.Bclls.GerminalCenter_Prolif.AICDA_CDK1, 36873.12251, CD, Bclls,
CD.B/DZ.AICDA.CDK1; CD.Bclls.GerminalCenter_Prolif.HOPX_IGHA1_IGHG1, 28261.32002, CD, Bclls, CD.B/DZ.HIST1H1B.MKI67;
CD.Bclls.GerminalCenter_Prolif.IGHA1_DDX21, 18027.96554, CD, Bclls, CD.B/GC.RRM2.ZWINT; CD.Bclls.GerminalCenter_Prolif.p19, 16002.9146, CD, Bclls,
CD.B/GC.TYMS.MKI67; CD.Bclls.GerminalCenter_Prolif.p19_CDC20_TOP2A_CENPA, 34304.19846, CD, Bclls, CD.B/GC.CENPE.TOP2A;
CD.Bclls.GerminalCenter_Prolif.p19_PCNA_LTF, 24905.35098, CD, Bclls, CD.B/GC.HIST1H1A.RRM2; CD.Bclls.GerminalCenter_Prolif.p19_STXBP6, 6109.563231, CD, Bclls,
CD.B/GC.MKI67.RRM2.1; CD.Bclls.GerminalCenter_Prolif.p28_MX1_OAS1_IRF7_AICDA, 25862.77626, CD, Bclls, CD.B/GC.RRM2.TK1;
CD.Bclls.GerminalCenter_Prolif.PLAC8_HLA-A_TACI, 14182.78379, CD, Bclls, CD.B/GC.MKI67.RRM2; CD.Bclls.GerminalCenter_Prolif.SRM_NME1, 12939.1341, CD, Bclls,
CD.B/GC.RRM2.TYMS; CD.Bclls.GerminalCenter.CCR7_CD83_HLA-DQ_SLAMF7, 18552.87754, CD, Bclls, CD.B/LZ.CCL22.NPW;
CD.Bclls.GerminalCenter.CD79B_PTPRC_CD27, 11539.27199, CD, Bclls, CD.B/GC.KIAA0101.GINS2; CD.Bclls.GerminalCenter.doublets_Tclls, 3913.6556, CD, Doublets/Bclls,
CD.Doublets/B.UBE2C.STMN1; CD.Bclls.GerminalCenter.HMGB1_H2AFZ, 4583.204791, CD, Bclls, CD.B.UBE2C.KIAA0101; CD.Bclls.GPR183_TACI, 334.8401939, CD, Bclls,
CD.B.RPS29.RPS21; CD.Bclls.HLA-DP_IGHD_FCER2_CD79A, 409.4015067, CD, Bclls, CD.B.RPS10.FCER2; CD.Bclls.HLA-DR_CD69, 487.774149, CD, Bclls, CD.B.MT-CO2.MT-
CO1; CD.Bclls.IGHD_CD74_IGHG_IGHA_CD69, 949.628242, CD, Bclls, CD.B.MT-ND4L.IGHG1; CD.Bclls.IGLC2_HLA-DR_CD69_CD83_IGHD_IGHG1.HLA-DQ_MX1_IRF7,
1853.110866, CD, Bclls, CD.B.IFI44L.IGLC2; CD.Bclls.IGLC2_HLA-DR_CD69_CD83_IGHD_IGHG1.IGKC_IGHG1_IGHA1_IGHA2, 456.2070877, CD, Bclls, CD.B.IGHG1.IGLC2;
CD.Bclls.IGLC2_HLA-DR_CD69_CD83_IGHD_IGHG1.IGLC3_IGLC2, 665.5358177, CD, Bclls, CD.B.IGLC2.HLA-DRB1; CD.Bclls.IGLC2_IGLC3_IGHG4_IGHA2_IGHA1_CD69,
1336.834872, CD, Bclls, CD.B.IGLC3.IGLC2; CD.Bclls.IGLC3.doublets_epithelial, 559.6327546, CD, Doublets/Bclls, CD.Doublets/B.IGLC3.DEFA5; CD.Bclls.IGLC3.HLA-
DR_HLA-DQ_MEF2C_SELL, 642.0900057, CD, Bclls, CD.B.IGLC3.HLA-DRB1; CD.Bclls.IGLC3.IFI44L_MX1_IFITM2, 537.5938702, CD, Bclls, CD.B.IGLC3.IFI44L;
CD.Bclls.IGLC3.IGHA_CD27, 1235.507218, CD, Bclls, CD.B.IGLC3.JCHAIN; CD.Bclls.IGLC3.ribosomal, 501.1840809, CD, Bclls, CD.B.IGLC3.RP11-347P51;
CD.Bclls.interferon_high.HOPX_IGLC2_CD19_IL10_IRF7, 9375.48581, CD, Bclls, CD.B.HCK.DUSP4; CD.Bclls.interferon_high.IGHA1_AIM2_CD27, 4718.668256, CD, Bclls,
CD.B.FCRL4.OSTN-AS1; CD.Bclls.interferon_high.p26_CXCR4_CD69_SELL, 2723.728745, CD, Bclls, CD.B.IFI44L.ISG15; CD.Bclls.plasmablast_IGHA_JCHAIN_CXCR4,
1459.246672, CD, Bclls, CD.B.IGHA2.JCHAIN; CD.Bclls.ribosomal, 94.92228438, CD, Bclls, CD.B.ACTB.JCHAIN; CD.Endth.arteriolar_SEMA3G_HEY1_PCSK5, 3048.406761,
CD, Endth, CD.Endth/Art.SEMA3G.SSUH2; CD.Endth.capillary_PLVAP_FLT1, 623.4465056, CD, Endth, CD.Endth/Cap.CD36.FLT1; CD.Endth.doublets_bcll, 22954.21212, CD,
Doublets/Endth, CD.Doublets/Endth.CD79A.VPREB3; CD.Endth.doublets_myeloid, 15595.86145, CD, Doublets/Endth, CD.Doublets/Endth.FCN1.RP11-1143G94;
CD.Endth.doublets_Tclls, 4017.712656, CD, Doublets/Endth, CD.Doublets/Endth.TRAC.CD2; CD.Endth.IFI6_ISG15_LY6E, 1407.586443, CD, Endth, CD.Endth.IFI6.ISG15;
CD.Endth.lymphatics_CCL21_LYVE1_PROX1.GIMAP7_CCL21_CLEC4G, 5714.70253, CD, Endth, CD.Endth/Lymph.CCL21.LYVE1;
CD.Endth.lymphatics_CCL21_LYVE1_PROX1.HES1_LY6H, 4136.58466, CD, Endth, CD.Endth/Lymph.LY6H.CCL21;
CD.Endth.lymphatics_CCL21_LYVE1_PROX1.mitochondrial, 1496.72081, CD, Endth, CD.Endth/Lymph.CCL21.PROX1;
CD.Endth.lymphatics_CCL21_LYVE1_PROX1.MMRN1_HLA-DR, 2094.604509, CD, Endth, CD.Endth/Lymph.EFEMP1.LYVE1;
CD.Endth.lymphatics_CCL21_LYVE1_PROX1.SOD2_IL3RA_BACE2, 3323.715882, CD, Endth, CD.Endth/Lymph.EFEMP1.CCL21;
CD.Endth.lymphatics_CCL21_LYVE1_PROX1.spillover_ig, 1311.530471, CD, Endth, CD.Endth/Lymph.CCL21.DKK3; CD.Endth.mitochondrial, 375.8684688, CD, Endth,
CD.Endth.MT-CO3.MT-CO2; CD.Endth.mural_cells_FRZB_NOTCH3_PDGFRB, 30408.73481, CD, Endth, CD.Endth/Mural.HIGD1B.NDUFA4L2;
CD.Endth.postcapillary_venule_ACKR1_SELP_MADCAM1.CLEC3B_POSTN, 574.5732046, CD, Endth, CD.Endth/Ven.POSTN.ADGRG6;
CD. Endth.postcapillary_venule_ACKR1_SELP_MADCAM1.JUN_FOS_IRF1, 693.4410299, CD, Endth, CD.Endth/Ven.CCL23.ACKR1;
CD.Endth.postcapillary_venule_ACKR1_SELP_MADCAM1.mitochondrial, 152.4161283, CD, Endth, CD.Endth/Ven.ACKR1.ADGRG6;
CD. Endth.postcapillary_venule_ACKR1_SELP_MADCAM1.P2RY6_COL4A1_SELL, 10551.05088, CD, Endth, CD.Endth/Ven.LAMP3.LIPG;
CD. Endth.postcapillary_venule_ACKR1_SELP_MADCAM1.ribosomal, 541.2261728, CD, Endth, CD.Endth/Ven.POSTN.ACKR1;
CD.Endth.postcapillary_venule_ACKR1_SELP_MADCAM1.TXNIP_CD320_PLAT, 471.9626593, CD, Endth, CD.Endth/Ven.ADGRG6.ACKR1;
CD.Endth.postcapillary_venule_ACKR1_SELP_MADCAM1.ZFP36_FOS_JUN, 497.9982891, CD, Endth, CD.Endth/Ven.CPE.GALNT15;
CD.Endth.spillover_ig.doublets_epithelial, 1342.999835, CD, Doublets/Endth, CD.Doublets/Endth.PRAP1.FABP6; CD.Endth.spillover_ig.FCN3_SPARC_VAMP5,
35.25366721, CD, Doublets/Endth, CD.Doublets/Endth.JCHAIN.IGKC; CD.Endth.spillover_ig.mitochondrial, 48.3629474, CD, Doublets/Endth, CD.Doublets/Endth.DEFA5;
CD.Endth.spillover_ig.postcapillary_venule_ACKR1_NPC2_CLU, 33.23900926, CD, Doublets/Endth, CD.Doublets/Endth.IGLC2.JCHAIN; CD.Epith_Paneth, 2.14E+301, CD,
Epith, CD.Paneth.DEFA6.ITLN2; CD.Epith.crypt_based_secretory_REG1B_CD24_OLFM4.GSTA1_LIPH, 1124.821042, CD, Epith, CD.Secretory.GSTA1.REG1B;
CD.Epith.crypt_based_secretory_REG1B_CD24_OLFM4.IFI6_MX1_LY6E, 4963.712577, CD, Epith, CD.Secretory.REG3G.IFI6;
CD.Epith.crypt_based_secretory_REG1B_CD24_OLFM4.PHGDH_KRT20_LGALS3, 1383.942542, CD, Epith, CD.EC.MT1H.PHGDH;
CD.Epith.crypt_based_secretory_REG1B_CD24_OLFM4.proliferating_PLA2G2A, 1143.042453, CD, Epith, CD.Prolif/Secretory.REG1B.MGST1;
CD.Epith.crypt_based_secretory_REG1B_CD24_OLFM4.ribosomal, 243.3846678, CD, Epith, CD.Secretory.REG1B.REG1A;
CD.Epith.crypt_based_secretory_REG1B_CD24_OLFM4.StemCell_LGR5_NUPR1, 490.4291936, CD, Epith, CD.EC.NUPR1.LCN2;
CD.Epith.DEFA5_DEFA6_REG3A.CFTR_OLFM4, 478.9034841, CD, Epith, CD.EC.CFTR.OLFM4; CD.Epith.DEFA5_DEFA6_REG3A.IGFBP7_CXCL14, 153.6419993, CD,
Doublets/Epith, CD.Doublets/Epith.ADAMDEC1.IGFBP7; CD.Epith.DEFA5_DEFA6_REG3A.MIF_HMGB1_C15orf48, 870.3626356, CD, Epith, CD.EC.MIF.MTRNR2L8;
CD.Epith. DEFA5_DEFA6_REG3A.REG3A_REG1B_LYZ, 16.87311176, CD, Epith, CD.EC.OLFM4.MT-ND2; CD.Epith.DEFA5_DEFA6_REG3A.spillover_ig, 315.5891416, CD,
Doublets/Epith, CD.Doublets/Epith.JCHAIN.IGHA1; CD.Epith.doublets_bcll.doublets_bcll, 24863.42667, CD, Doublets/Epith, CD.Doublets/Epith.MS4A1.CD79A;
CD.Epith.doublets_bcll.doublets_DC, 51053.76464, CD, Doublets/Epith, CD.Doublets/Epith.CLEC9A.CPVL; CD.Epith.doublets_bcll.doublets_myeloid, 142.7494673, CD,
Doublets/Epith, CD.Doublets/Epith.CXCR4.PTPRC; CD.Epith.doublets_bcll.PIGR_IFI6_CCL2, 1259.438044, CD, Epith, CD.EC.TCL1A.IFI6; CD.Epith.doublets_Tcll,
12106.44471, CD, Doublets/Epith, CD.Doublets/Epith.CD2.TRAC; CD.Epith.early_entero_FABP4_ALDOB_ANPEP.APOA_SLC26A3_DEFA5, 6344.693487, CD, Epith,
CD.EC.FABP6.SLC26A3; CD.Epith.early_entero_FABP4_ALDOB_ANPEP.DUOX2_MUC1_mitochondrial, 901.5278839, CD, Epith, CD.EC.ANPEP.DUOX2;
CD.Epith.early_entero_FABP4_ALDOB_ANPEP.MGST1_AGR2_EPCAM.APOB_ZG16, 6073.080284, CD, Epith, CD.EC.GSTA2.AADAC;
CD.Epith.early_entero_FABP4_ALDOB_ANPEP.MGST1_AGR2_EPCAM.CCDC59_CBR1, 2522.158283, CD, Epith, CD.EC.GSTA2.TMPRSS15;
CD.Epith.early_entero_FABP4_ALDOB_ANPEP.MGST1_AGR2_EPCAM.IFI6_IFI27_IFI44L, 1476.201674, CD, Epith, CD.EC.ARHGDIG.SULT1E1;
CD.Epith.early_entero_FABP4_ALDOB_ANPEP.MGST1_AGR2_EPCAM.LYZ_REG4, 1620.741196, CD, Epith, CD.EC.CA1.TMPRSS15;
CD. Epith.early_entero_FABP4_ALDOB_ANPEP.MGST1_AGR2_EPCAM.MT1, 428.8101291, CD, Epith, CD.EC.AKR1C1.TSPAN1;
CD.Epith.early_entero_FABP4_ALDOB_ANPEP.MGST1_AGR2_EPCAM.PLA2G2A_FOLH1_REG1B, 1734.840662, CD, Epith, CD.EC.TMPRSS15.FAM8A1;
CD.Epith.early_entero_FABP4_ALDOB_ANPEP.MGST1_AGR2_EPCAM.SLC28A2_ST6GAL1, 7029.864492, CD, Epith, CD.EC.SLC28A2.GSTA2;
CD.Epith.early_entero_FABP4_ALDOB_ANPEP.MGST1_AGR2_EPCAM.spillover_ig, 1091.262387, CD, Epith, CD.EC.GSTA2.CES3;
CD.Epith.early_entero_FABP4_ALDOB_ANPEP.OAT_SULT1A1, 5142.296553, CD, Epith, CD.EC.RBP2.CYP3A4; CD.Epith.early_entero_FABP4_ALDOB_ANPEP.PLCG2_SOX4,
1798.289219, CD, Epith, CD.EC.FABP6.PLCG2; CD.Epith.early_entero_FABP4_ALDOB_ANPEP.REG1B_MT, 3782.424682, CD, Epith, CD.EC.RBP2.ALPI;
CD.Epith.early_entero_FABP4_ALDOB_ANPEP.ribosomal, 514.3307323, CD, Epith, CD.EC.FABP1.ADIRF; CD.Epith.enteroendocrine_CHGA_CSG5.mitochondrial,
60531.19535, CD, Epith, CD.Enteroendocrine.TPH1.CHGA; CD.Epith.enteroendocrine_CHGA_CSG5.NEUROG3_HES6_CD24, 2.38E+302, CD, Epith,
CD.Enteroendocrine.NEUROG3.MLN; CD.Epith.enteroendocrine_CHGA_CSG5.PYY_GCG_CCK, 5.65E+301, CD, Epith, CD.Enteroendocrine.ONECUT3.CCK;
CD.Epith.enteroendocrine_CHGA_CSG5.TPH1_CES1_CHGA, 4.21E+301, CD, Epith, CD.Enteroendocrine.TFPI2.TPH1; CD.Epith.goblet_MUC6_BPIFB1_MUC5B, 7.08E+302,
CD, Epith, CD.Goblet.BPIFB1.AQP5; CD.Epith.goblet_SPINK4_TFF3_ITLN1.AGR2_PDIA4, 2144.559906, CD, Epith, CD.Goblet.REG4.SPINK4;
CD.Epith.goblet_SPINK4_TFF3_ITLN1.APOA_FABP6_DEFA5, 3695.934722, CD, Epith, CD.Goblet.FCGBP.CLCA1; CD.Epith.goblet_SPINK4_TFF3_ITLN1.doublets_Tcll,
2894.807164, CD, Doublets/Epith, CD.Doublets/Epith.ITLN1.CLCA1; CD.Epith.goblet_SPINK4_TFF3_ITLN1.ITLN1_CLCA1_REG4, 15367.50216, CD, Epith,
CD.Goblet.ITLN1.CLCA1; CD.Epith.goblet_SPINK4_TFF3_ITLN1.mitochondrial, 5194.671668, CD, Epith, CD.Goblet.FCGBP.SPINK4;
CD.Epith.goblet_SPINK4_TFF3_ITLN1.OLFM4_CCL25_CD24.HLA-A_CD24_LGALS9, 1469.859641, CD, Epith, CD.Goblet.HES6.COLCA2;
CD.Epith.goblet_SPINK4_TFF3_ITLN1.OLFM4_CCL25_CD24.ITLN1, 1942.383538, CD, Epith, CD.Goblet.ITLN1.CLCA1.1;
CD.Epith.goblet_SPINK4_TFF3_ITLN1.OLFM4_CCL25_CD24.mitochondrial, 570.4195482, CD, Epith, CD.Goblet.FCGBP.HES6;
CD.Epith.goblet_SPINK4_TFF3_ITLN1.PLCG2_MAFB, 3087.593224, CD, Epith, CD.Goblet.FCGBP.ITLN1; CD.Epith.goblet_SPINK4_TFF3_ITLN1.proliferating, 9417.528883,
CD, Epith, CD.Goblet.CENPA.UBE2C; CD.Epith.goblet_SPINK4_TFF3_ITLN1.REG3A_LYZ_LCN2, 4668.696862, CD, Epith, CD.Goblet.RETNLB.ITLN1;
CD. Epith.goblet_SPINK4_TFF3_ITLN1.S100P_MT1, 21677.24876, CD, Epith, CD.Goblet.S100P.TFF1; CD.Epith.goblet_SPINK4_TFF3_ITLN1.TFF1_SPINK4_B3GNT7_FFAR4,
26223.48126, CD, Epith, CD.Goblet.TFF1.TPSG1; CD.Epith.goblet_SPINK4_TFF3_ITLN1.TM4SF20_NR1H4_CES2, 5857.31207, CD, Epith, CD.Goblet.S100P.FCGBP;
CD.Epith.late_enterocyte_APOA_APOC_APOB_p14.MT1, 11490.17986, CD, Epith, CD.EC.APOC3.APOA4; CD.Epith.late_enterocyte_APOA_APOC_APOB_p14.0AT_SLC2A2,
22424.82558, CD, Epith, CD.EC.APOB.APOC3; CD.Epith.late_enterocyte_APOA_APOC_APOB_p14.PLCG2_HES1, 25672.76053, CD, Epith, CD.EC.APOC3.CUBN;
CD.Epith.MGST1_CCL25_CD9_EPCAM.MCells_SPIB_SOX8_CCL23_CCL20, 83995.68275, CD, Epith, CD.Mcell.CCL23.SPIB; CD.Epith.MGST1_CCL25_CD9_EPCAM.proliferating, 4509.216972,
CD, Epith, CD.Prolif/EC.UBE2C.CCNB2; CD.Epith.MGST1_CCL25_CD9_EPCAM.ZFP36_OAT.CD74_IL2RG_CCL15, 4616.052827, CD, Epith,
CD.EC.LECT1.FAM26F; CD.Epith.MGST1_CCL25_CD9_EPCAM.ZFP36_OAT.doublets_plasma_IFITM1, 1160.028681, CD, Epith, CD.EC.UBD.IFITM1;
CD.Epith.MGST1_CCL25_CD9_EPCAM.ZFP36_OAT.GSTA1_MT, 489.4271267, CD, Epith, CD.EC.ADH1C.GSTA1;
CD.Epith.MGST1_CCL25_CD9_EPCAM.ZFP36_OAT.LYZ_S100P_REG3A, 203.3041536, CD, Epith, CD.EC.LYZ.OLFM4;
CD.Epith. MGST1_CCL25_CD9_EPCAM.ZFP36_OAT.MCells_SPIB_LTB_CCL25, 910.2906602, CD, Epith, CD.Mcell.CSRP2.SPIB;
CD.Epith.MGST1_CCL25_CD9_EPCAM.ZFP36_OAT.MT1_GSTA1_DHFR, 571.5620741, CD, Epith, CD.EC.ADH1C.RPS4Y1;
CD.Epith.MGST1_CCL25_CD9_EPCAM.ZFP36_OAT.StemCell_LGR5_SOX4, 3617.89084, CD, Epith, CD.EpithStem.LINC00176.RPS4Y1;
CD.Epith.MGST1_CCL25_CD9_EPCAM.ZFP36_OAT.TFRC_C15orf48, 455.248187, CD, Epith, CD.EC.HSPA1B.PSMA2; CD.Epith.mitochondrial, 1091.914334, CD, Epith,
CD.EC.MTRNR2L1.MT-ND3; CD.Epith.ribosomal_REG1A.APOA_MIF, 87.3900808, CD, Epith, CD.EC.MIF.AC0904981; CD.Epith.ribosomal_REG1A.doublets_plasma,
100.7508625, CD, Doublets/Epith, CD.Doublets/Epith.IGHA1.IGKC; CD.Epith.ribosomal_REG1A.HMGCS2_KLF4_OSR2, 978.0379448, CD, Epith, CD.EC.ADH1C.EDN1;
CD.Epith.ribosomal_REG1A.IFI6_IFI27_IFI44L, 306.1136687, CD, Epith, CD.EC.IFI6.IFI27; CD.Epith.ribosomal_REG1A.mitochondrial, 102.6565726, CD, LowQual/Epith,
CD.EC.MT-CO2.MT-CO3; CD.Epith.ribosomal_REG1A.MT1, 114.116691, CD, Epith, CD.EC.MT1H.MT1G; CD.Epith.ribosomal_REG1A.proliferating, 85.31509932, CD, Epith,
CD.Prolif/EC.UBE2C.HMGB2; CD.Epith.ribosomal_REG1A.ribosomal, 27.68754309, CD, LowQual/Epith, CD.EC.RPS15.RPL10;
CD.Epith.ribosomal_REG1A.SMOC2_ASCL2_CXCL3, 239.4152469, CD, Epith, CD.EC.SMOC2.ASCL2; CD.Epith.SOX4_HES1_HEXIM1_PLCG2.mitochondrial, 1024.904384, CD,
Epith, CD.TA.PLCG2.SOX4; CD.Epith.SOX4_HES1_HEXIM1_PLCG2.p14_APOA_APOC_DEFA, 1509.853979, CD, Epith, CD.EC.PLCG2.MTRNR2L8;
CD.Epith.SOX4_HES1_HEXIM1_PLCG2.PLCG2_ELF3, 2630.752324, CD, Epith, CD.EC.PLCG2.MAFB; CD.Epith.SOX4_HES1_HEXIM1_PLCG2.ribosomal, 737.4966631, CD,
Epith, CD.EC.PLCG2.RP11-727F159; CD.Epith.SOX4_HES1_HEXIM1_PLCG2.Tuft_HPGDS_TRPM5, 4.83E+301, CD, Epith, CD.EC.GNAT3.TRPM5;
CD.Fibroblast.ADAMDEC1_CXCL12_WNT2B, 1619.311574, CD, Fibro, CD.Fibro.ADAMDEC1.C7; CD.Fibroblast.ADAMEC1_IDO1.AGT_PCDH18, 27.2608709, CD, Fibro,
CD.Fibro.PCDH18.APOE; CD.Fibroblast.ADAMEC1_IDO1.COL3A1_CXCL1, 5.571364742, CD, Fibro, CD.Fibro.MT-ND4L.NEAT1; CD.Fibroblast.ADAMEC1_IDO1.CXCL14,
67.28276852, CD, Fibro, CD.Fibro.CXCL14.DEFA5; CD.Fibroblast.ADAMEC1_IDO1.doublets_epithelial, 527.1017291, CD, Doublets/Fibro,
CD.Doublets/Fibro.APOA4.APOA1; CD.Fibroblast.ADAMEC1_IDO1.doublets_plasma, 29.25438446, CD, Doublets/Fibro, CD.Doublets/Fibro.JCHAIN.IGHA1;
CD.Fibroblast.ADAMEC1_IDO1.IFI6_IFI27, 101.2165712, CD, Fibro, CD.Fibro.IFI6.IFI44L; CD.Fibroblast.ADAMEC1_IDO1.IGF1_COL6A5_APOD, 134.1822883, CD, Fibro,
CD.Fibro.COL6A5.SLITRK6; CD.Fibroblast.ADAMEC1_IDO1.mitochondrial, 25.51878327, CD, Fibro, CD.Fibro.DEFA6.DEFA5;
CD.Fibroblast.ADAMEC1_IDO1.PRELP_OGN_IL33, 54.95400128, CD, Fibro, CD.Fibro.PRELP.OGN; CD.Fibroblast.ADAMEC1_IDO1.TAGLN_ACTA2, 91.73803068, CD, Fibro,
CD.Fibro.PHGDH.STMN2; CD.Fibroblast.ADAMEC1_IDO1.TFPI2_CCL11, 39.58701265, CD, Fibro, CD.Fibro.RPS4Y1.DEFA5;
CD.Fibroblast.ADAMEC1_IDO1.WFDC1_CLEC14A, 154.2507602, CD, Fibro, CD.Fibro.WFDC1.PTN; CD.Fibroblast.ADAMEC1_IDO1.WNT2B_CXCL6_IGFBP3, 92.19684393,
CD, Fibro, CD.Fibro.STMN2.FABP5; CD.Fibroblast.C3_CTSH_CD24.CCL19_CCL21_TNFRSF11B_GREM1, 26854.38022, CD, Fibro, CD.Fibro.CCL21.CCL19;
CD.Fibroblast.C3_CTSH_CD24.CXCL14_CXCL10_IDO1, 2894.348201, CD, Fibro, CD.Fibro.TNFSF11.CD24;
CD.Fibroblast.C3_CTSH_CD24.folliculardendritic_FDCSP_CR2_SPIB_CXCL13, 1.81E+302, CD, Fibro, CD.Fibro/fDC.FDCSP.CXCL13; CD.Fibroblast.CFD_PTGDS_CCL13,
2863.646282, CD, Fibro, CD.Fibro.TFPI2.CCL13; CD.Fibroblast.CXCL6_CXCL1_CCL11.CCL19_SOD3.CHI3L1_CXCL6, 1541.654033, CD, Fibro, CD.Fibro.CHI3L1.CXCL3;
CD.Fibroblast.CXCL6_CXCL1_CCL11.CCL19_SOD3.GSN_IFI27_IRF7, 190.437808, CD, Fibro, CD.Fibro.CCL19.IRF7;
CD.Fibroblast.CXCL6_CXCL1_CCL11.CCL19_SOD3.OGN_C7, 744.0700333, CD, Fibro, CD.Fibro.OGN.C1QTNF3; CD.Fibroblast.CXCL6_CXCL1_CCL11.CCL8_CCL13,
896.3302718, CD, Fibro, CD.Fibro.CCL13.CXCL6; CD.Fibroblast.CXCL6_CXCL1_CCL11.COL6A5_ADAMDEC1, 762.0980508, CD, Fibro, CD.Fibro.COL6A5.CXCL6;
CD.Fibroblast.CXCL6_CXCL1_CCL11.CXCL13_CHI3L1, 102066.6097, CD, Fibro, CD.Fibro.CA12.SFRP2; CD.Fibroblast.CXCL6_CXCL1_CCL11.IGF2_RAMP1.CXCL14_LUM,
591.7783921, CD, Fibro, CD.Fibro.IGF2.CXCL6; CD.Fibroblast.CXCL6_CXCL1_CCL11.IGF2_RAMP1.MIF_IFI27, 161.49488, CD, Fibro, CD.Fibro.CD24.IGHG4;
CD.Fibroblast.CXCL6_CXCL1_CCL11.IGF2_RAMP1.RAMP1, 935.9895227, CD, Fibro, CD.Fibro.RAMP1.IGHG1; CD.Fibroblast.CXCL6_CXCL1_CCL11.TNFRSF11B_COL18A1,
5558.643351, CD, Fibro, CD.Fibro.TNFRSF11B.TFPI2; CD.Fibroblast.doublets_epithelial, 5415.789453, CD, Doublets/Fibro, CD.Doublets/Fibro.APOA1.FABP6;
CD.Fibroblast.doublets_myeloid, 29777.86416, CD, Doublets/Fibro, CD.Doublets/Fibro.IL1B.CLEC10A; CD.Fibroblast.doublets_Tclls.ADAMDEC1_IDO1, 356.70495, CD,
Doublets/Fibro, CD.Doublets/Fibro.LTB.VPREB3; CD.Fibroblast.doublets_Tclls.CACL1_CFD, 695.7874608, CD, Doublets/Fibro, CD.Doublets/Fibro.CST7.NKG7;
CD.Fibroblast.doublets_Tclls.CTGF_APOD, 395.9816354, CD, Doublets/Fibro, CD.Doublets/Fibro.LTB.TRAC; CD.Fibroblast.doublets_Tclls.CXCL1_CRYBG3, 496.6456824,
CD, Doublets/Fibro, CD.Doublets/Fibro.PTPRC.GZMA; CD.Fibroblast.doublets_Tclls.CYGB_KLN53, 902.4554795, CD, Doublets/Fibro,
CD.Doublets/Fibro.AC0925804.KLRB1; CD.Fibroblast.doublets_Tclls.doublets_bcll, 13343.71089, CD, Doublets/Fibro, CD.Doublets/Fibro. VPREB3.CD79A;
CD.Fibroblast.doublets_Tclls.doublets_mast, 81759.0793, CD, Doublets/Fibro, CD.Doublets/Fibro.MS4A2.HPGDS; CD.Fibroblast.doublets_Tclls.doublets_Tcll,
10633.26238, CD, Doublets/Fibro, CD.Doublets/Fibro.GNLY.TRDC; CD.Fibroblast.doublets_Tclls.FIGF_CPM, 990.1100043, CD, Doublets/Fibro, CD.Hirschsprung.FIGF.CPM;
CD.Fibroblast.doublets_Tclls.GSN_CFD, 279.7524335, CD, Doublets/Fibro, CD.Doublets/Fibro.AC0925804.KLRB1; CD.Fibroblast.doublets_Tclls.LUM_MFAP4,
649.7305838, CD, Doublets/Fibro, CD.Doublets/Fibro.CD3D.CD69; CD.Fibroblast.doublets_Tclls.MARCH1_SERPINA9_doublets_bcll, 4505.549498, CD, Doublets/Fibro,
CD.Doublets/Fibro.MS4A1.MARCH1; CD.Fibroblast.doublets_Tclls.NR2F2_IL33, 1176.9924, CD, Doublets/Fibro, CD.Doublets/Fibro.CCL5.CD8B;
CD.Fibroblast.doublets_Tclls.OAS1_IFI44L, 835.8577299, CD, Doublets/Fibro, CD.Doublets/Fibro.NKG7.LAG3; CD.Fibroblast.doublets_Tclls.OGN_EMCN, 1225.983865, CD,
Doublets/Fibro, CD.Doublets/Fibro.KLRB1.IL7R; CD.Fibroblast.doublets_Tclls.proliferating, 305.5702778, CD, Doublets/Fibro, CD.Doublets/Fibro.RGS1.CXCR4;
CD.Fibroblast.doublets_Tclls.PTPRS_CTNNB1, 1552.197099, CD, Doublets/Fibro, CD.Doublets/Fibro.NKG7.AC0925804; CD.Fibroblast.doublets_Tclls.SRPX_AMBRA1,
658.426331, CD, Doublets/Fibro, CD.Doublets/Fibro.GNLY.KLRD1; CD.Fibroblast.doublets_Tclls.Tclls, 1440.369354, CD, Doublets/Fibro, CD.Doublets/Fibro.TRAC.CD3D;
CD.Fibroblast.doublets_Tclls.TNFRSF11_TFPI2, 650.9903748, CD, Doublets/Fibro, CD.Doublets/Fibro.KLRB1.TFPI2; CD.Fibroblast.doublets_Tclls.WFDC1_TLR5,
877.7655395, CD, Doublets/Fibro, CD.Doublets/Fibro.KLRB1.GATA3; CD.Fibroblast.F3_EDNRB_FRZB.AGT_CD74_CD82, 1180.26328, CD, Fibro, CD.Fibro.AGT.F3;
CD.Fibroblast.F3_EDNRB_FRZB.COL6A2_MMP11_COL1A1, 8306.250855, CD, Fibro, CD.Fibro.FIGF.MMP11; CD.Fibroblast.F3_EDNRB_FRZB.doublets_epithelial,
1918.065075, CD, Doublets/Fibro, CD.Doublets/Fibro.VSTM2A.FABP6; CD.Fibroblast.F3_EDNRB_FRZB.LUM_APOE, 1117.904308, CD, Fibro, CD.Fibro.GDF15.EGR1;
CD.Fibroblast.F3_EDNRB_FRZB.PTGDS_LY6H_FN1.ACTA2_CTGF, 4530.554695, CD, Fibro, CD.Fibro.LY6H.PAPPA2;
CD.Fibroblast. F3_EDNRB_FRZB.PTGDS_LY6H_FN1.ALCAM_SPARC, 1410.16694, CD, Fibro, CD.Fibro.LY6H.PCSK6;
CD.Fibroblast. F3_EDNRB_FRZB.PTGDS_LY6H_FN1.mitochondrial, 92.29619461, CD, Fibro, CD.Fibro.F3.SOX6; CD.Fibroblast.F3_EDNRB_FRZB.spillover_ig.FBXW2_CCL2,
186.9410779, CD, Fibro, CD.Fibro.CPM.HSD17B2; CD.Fibroblast.F3_EDNRB_FRZB.spillover_ig.IGFBP5_PTGDS_APOD, 801.1587079, CD, Fibro, CD.Fibro.PCSK6.FIGF;
CD.Fibroblast.F3_EDNRB_FRZB.spillover_ig.mitochondrial, 43.15598563, CD, Doublets/Fibro, CD.Doublets/Fibro.JCHAIN.MT-ND1;
CD.Fibroblast. F3_EDNRB_FRZB.spillover_ig.PLAT_NPY, 1110.706323, CD, Fibro, CD.Fibro.NPY.HSD17B2; CD.Fibroblast.glial_S100B_SPP1_PLP1.COL28A1_CAB39L,
15520.39569, CD, Fibro, CD.Glial.COL28A1.SPP1; CD.Fibroblast.glial_S100B_SPP1_PLP1.doublets_Tclls, 8107.8814, CD, Doublets/Fibro, CD.Doublets/Glial.S100B.NRXN1;
CD.Fibroblast.glial_S100B_SPP1_PLP1.FOS_JUN, 7799.100975, CD, Fibro, CD.Glial.SPP1.NRXN1; CD.Fibroblast.glial_S100B_SPP1_PLP1.IDO1_CCL2, 17217.07408, CD, Fibro,
CD.Glial.SPP1.NOV; CD.Fibroblast.glial_S100B_SPP1_PLP1.IGFBP7_SPARC, 19879.58542, CD, Fibro, CD.Glial.S100B.SPP1;
CD.Fibroblast.glial_S100B_SPP1_PLP1.mitochondrial, 731.6093208, CD, Fibro, CD.Glial.S100B.SPP1.1; CD.Fibroblast.glial_S100B_SPP1_PLP1.ribosomal, 4534.539343, CD,
Fibro, CD.Glial.S100B.PLP1; CD.Fibroblast.glial_S100B_SPP1_PLP1.XKR4_XIST, 11855.04916, CD, Fibro, CD.Glial.XKR4.S100B;
CD.Fibroblast.mitochondrial_doublets.C3_CXCL14_GBP2, 123.2949835, CD, Doublets/Fibro, CD.Doublets/Fibro.DEFA5.C3;
CD.Fibroblast.mitochondrial_doublets.mitochondrial, 148.4823027, CD, Doublets/Fibro, CD.Doublets/Fibro.MT-CO1.MT-CO3;
CD.Fibroblast.mitochondrial_doublets.ribosomal, 41.26820311, CD, Doublets/Fibro, CD.Doublets/Fibro.MT-ND1.MT-CO2;
CD.Fibroblast.mitochondrial_doublets.XIST_CCL11_COL3A1, 40.64152624, CD, Doublets/Fibro, CD.Doublets/Fibro.APOA1.MT-CO3;
CD.Fibroblast.myofibroblast_ACTG2_MYH11, 78841.30997, CD, Fibro, CD.MyoFibro.MFAP5.SOSTDC1; CD.Fibroblast.NOTCH3_CD36, 24758.25147, CD, Fibro,
CD.Fibro.HIGD1B.RGS5; CD.Fibroblast.PLN_MYH11_NOTCH3, 3.52E+302, CD, Fibro, CD.Fibro.RERGL.NTRK2; CD.IGA_IGK_Plsma.doublets_epithelial, 13075.11534, CD,
Doublets/Plsma, CD.Doublets/IGA.PHGR1.KRT8; CD.IGA_IGK_Plsma.doublets_Tclls, 5159.175254, CD, Doublets/Plsma, CD.Doublets/IGA.KLRB1.IL32;
CD.IGA_IGK_Plsma.IGKV1-12, 1164.851176, CD, Plsma, CD.Plsma/IgA.IGHA2.IGKV1-12; CD.IGA_IGK_Plsma.IGKV1-12_IGKV4-1, 4435.222827, CD, Plsma,
CD.Plsma/IgA.IGKV1-12.IGHA2; CD.IGA_IGK_Plsma.IGLC3_IGHG3, 18.9378785, CD, Plsma, CD.Plsma/IgA.IGHA1.IGLV6-57;
CD.IGA_IGK_Plsma.inflam_macrophage_IL1B_CSF1R, 48897.61742, CD, Doublets/Plsma, CD.Doublets/IGA.C1QC.MS4A7; CD.IGA_IGK_Plsma.JUN_FOS_HSP90,
958.0300349, CD, Plsma, CD.Plsma/IgA.HSPA1B.HSPA1A; CD.IGA_IGK_Plsma.mitochondrial, 674.5457943, CD, Plsma, CD.Plsma/IgA.MT-ND5.MT-ND4L;
CD.IGA_IGK_Plsma.plasmacells, 494.464423, CD, Plsma, CD.Plsma/IgA.IGHA1.IGKC; CD.IGA_IGK_Plsma.proliferating, 37608.02027, CD, Plsma,
CD.Plsma/IgA.RRM2.TOP2A; CD.IGA_IGK_Plsma.ribo_CD79A, 638.3667502, CD, Plsma, CD.Plsma/IgA.AL9287683.RPL39; CD.IGA_IGL_Plsma.doublets_epithelial,
596.1344231, CD, Doublets/Plsma, CD.Doublets/IGA.PHGR1.IGLV2-23; CD.IGA_IGL_Plsma.doublets_fibroblast, 87215.15955, CD, Doublets/Plsma,
CD.Doublets/IGA.COL3A1.LUM; CD.IGA_IGL_Plsma.doublets_macrophage_Bcells, 1730.566615, CD, Doublets/Plsma, CD.Doublets/IGA.HLA-DPB1.HLA-DRA;
CD.IGA_IGL_Plsma.doublets_Tclls, 1519.955626, CD, Doublets/Plsma, CD.Doublets/IGA.KLRB1.CD3D; CD.IGA_IGL_Plsma.IGLL5, 7168.450584, CD, Plsma,
CD.Plsma/IgA.IGLL5.IGLV2-14; CD.IGA_IGL_Plsma.IGLV2-14, 6989.482034, CD, Plsma, CD.Plsma/IgA.IGLV2-14.IGLV3-19; CD.IGA_IGL_Plsma.IGLV6-57, 13049.03226, CD,
Plsma, CD.Plsma/IgA.IGLV1-51.IGLV10-54; CD.IGA_IGL_Plsma.JUN_FOS_HSP90, 622.9098025, CD, Plsma, CD.Plsma/IgA.IGLC3.IGLC2; CD.IGA_IGL_Plsma.plasmacells,
1586.265111, CD, Plsma, CD.Plsma/IgA.IGLC3.IGLC2.1; CD.IGA_IGL_Plsma.ribo_CD79A, 963.4238519, CD, Plsma, CD.Plsma/IgA.IGLC3.IGLC2.2; CD.IGG_Plsma,
40385.83494, CD, Plsma, CD.Plsma/IgG.IGHGP.IGHG2; CD.IGM_Plsma.doublets_Tcell_macrophage_Bcell, 2135.343449, CD, Doublets/Plsma, CD.Doublets/IGM.RGS13.HLA-
DRB5; CD.IGM_Plsma.IGKC, 1849.836654, CD, Plsma, CD.Plsma/IgM.IGHM.JCHAIN; CD.IGM_Plsma.IGLC, 1907.556478, CD, Plsma, CD.Plsma/IgM.IGHM.IGLV2-14;
CD.IGM_Plsma.MastCells, 135924.9011, CD, Plsma, CD.Mstcl.TPSAB1.HPGDS; CD.Mloid.AIF1_S100A4_FCN1_FCER1G_LYZ.CQ1A_CQ1C_macrophage.CCL3_CCL4,
84.37454894, CD, Mloid, CD.Mac.IGSF6.TXNIP; CD.Mloid.AIF1_S100A4_FCN1_FCER1G_LYZ.CQ1A_CQ1C_macrophage.CQ1A_CQ1C_macrophage, 69.50483313, CD, Mloid,
CD.Mac.C1QB.AIF1; CD.Mloid.AIF1_S100A4_FCN1_FCER1G_LYZ.CQ1A_CQ1C_macrophage.LYZ_TXN, 64.87259718, CD, Mloid, CD.Mac.LYZ.TXN;
CD.Mloid.AIF1_S100A4_FCN1_FCER1G_LYZ.CQ1A_CQ1C_macrophage.NFKB_IL1B_CXCL3_ICAM1, 93.48906382, CD, Mloid, CD.Mac.C1QB.CD14;
CD.Mloid.AIF1_S100A4_FCN1_FCER1G_LYZ.CQ1A_CQ1C_macrophage.S100A4_AIF1_FCN1_S100A9, 177.0781444, CD, Mloid, CD.Mac.AIF1.HBEGF;
CD.Mloid.AIF1_S100A4_FCN1_FCER1G_LYZ.HLADR_C1Q_CD68, 125.2674744, CD, Mloid, CD.Mac.C1QB.FCGR1A;
CD.Mloid.AIF1_S100A4_FCN1_FCER1G_LYZ.S100A10_CD55_KYNU.CD1C_FCER1A_FCGR2B, 1356.469117, CD, Mloid, CD.Mono/cDC2.FCN1.CD1C;
CD.Mloid.AIF1_S100A4_FCN1_FCER1G_LYZ.S100A10_CD55_KYNU.LST1_IFITM2_FCGR3A, 3878.224093, CD, Mloid, CD.Mono.FCN1.LYST;
CD.Mloid.AIF1_S100A4_FCN1_FCER1G_LYZ.S100A10_CD55_KYNU.ribosomal, 103.1246551, CD, Mloid, CD.Mono.FCN1.EIF4A3;
CD.Mloid.AIF1_S100A4_FCN1_FCER1G_LYZ.S100A10_CD55_KYNU.STAT1_C1Q_FCGR1A, 472.3664596, CD, Mloid, CD.Mono.FCN1.AREG;
CD.Mloid.AIF1_S100A4_FCN1_FCER1G_LYZ.S100A8_S100A9, 422.7386462, CD, Mloid, CD.Mono.FCN1.S100A4;
CD.Mloid.dendritic_CLEC10A_CD1C_FCER1A_ITGAX.CD209_CD163_VSIG4_AXL, 1346.783515, CD, Mloid, CD.Mono/cDC2.CLEC10A.SDS;
CD.Mloid.dendritic_CLEC10A_CD1C_FCER1A_ITGAX.CD209_VSIG4_C1Q_CD68_CSF1R, 573.9497877, CD, Mloid, CD.Mono/cDC2.CLEC10A.AREG;
CD.Mloid.dendritic_CLEC10A_CD1C_FCER1A_ITGAX.EGR1_patient11, 1345.193104, CD, Mloid, CD.cDC2.ZFAND2A.INHBA;
CD.Mloid.dendritic_CLEC10A_CD1C_FCER1A_ITGAX.HSP_patient11, 1734.699782, CD, Mloid, CD.cDC2.RALA.CD1C;
CD.Mloid.dendritic_CLEC10A_CD1C_FCER1A_ITGAX.IG_enriched_patient16, 3847.764938, CD, Mloid, CD.cDC2.FCER1A.CD1C;
CD.Mloid.dendritic_CLEC10A_CD1C_FCER1A_ITGAX.IL1R2_ribosomal, 2245.043859, CD, Mloid, CD.cDC2.CD1C.CD207;
CD.Mloid.dendritic_CLEC10A_CD1C_FCER1A_ITGAX.IL22RA2_SFTPD_LTB_IL23A, 30017.32588, CD, Mloid, CD.DC.LTB.IL22RA2;
CD.Mloid.dendritic_CLEC10A_CD1C_FCER1A_ITGAX.PSMA, 1207.114196, CD, Mloid, CD.cDC2.CD1C.AREG; CD.Mloid.dendritic_CLEC10A_CD1C_FCER1A_ITGAX.ribosomal,
3384.482282, CD, Mloid, CD.cDC2.CD1C.NDRG2; CD.Mloid.dendritic_CLEC10A_CD1C_FCER1A_ITGAX.S100A8_S100A9_patient11, 546.4589693, CD, Mloid,
CD.Mono.FCN1.HSPA1A; CD.Mloid.dendritic_CLEC10A_CD1C_FCER1A_ITGAX.TMEM176_CD1B_CD1E_CD40, 2019.602625, CD, Mloid, CD.cDC2.CLEC10A.CD1E;
CD.Mloid.dendritic_CLEC10A_CD1C_FCER1A_ITGAX.TXNIP_TNF_CX3CR1, 2011.330042, CD, Mloid, CD.cDC2.CLEC10A.FCGR2B;
CD.Mloid.dendritic_CLEC9A_BATF3_XCR1_IRF8, 48608.30682, CD, Mloid, CD.cDC1.CLEC9A.XCR1; CD.Mloid.dendritic_migratory_CCR7_FSCN1, 27852.46803, CD, Mloid,
CD.DC.CCR7.FSCN1; CD.Mloid.doublets_lymphoid, 1748.336034, CD, Doublets/Mloid, CD.Doublets/Mloid.CD3D.TRAC; CD.Mloid.doublets_lymphoid.doublet_bcll,
23497.64858, CD, Doublets/Mloid, CD.Doublets/Mloid.CD79A.MS4A1; CD.Mloid.doublets_lymphoid.doublets_Tcll, 7579.999153, CD, Doublets/Mloid,
CD.Doublets/Mloid.TRAC.CD2; CD.Mloid.doublets_lymphoid.FCER1G_IL1B.C1Q_APOE, 3002.016543, CD, Doublets/Mloid, CD.Doublets/Mac.CD3D.TRBC2;
CD.Mloid.doublets_lymphoid.FCER1G_IL1B.dendritic_CLEC10A_CD1C_FCER1A_CD207, 1312.492692, CD, Doublets/Mloid, CD.Doublets/cDC2.CD1C.CD3D;
CD.Mloid.doublets_lymphoid.FCER1G_IL1B.S100A8_S100A9_FCN1_AIF1, 2167.289657, CD, Doublets/Mloid, CD.Doublets/Mono.CD3D.IL7R;
CD.Mloid.macrophage_APOE_C1Q_CD63_CD14_AXL, 6660.412908, CD, Mloid, CD.Mac.APOE.PTGDS;
CD.Mloid.macrophage_chemokine.APOE_APOC1_CD163.doublets_plasma, 923.8587499, CD, Mloid, CD.Mac.CXCL2.CXCL3;
CD.Mloid.macrophage_chemokine.APOE_APOC1_CD163.macrophage_APOE_C1Q_CD63_FCER1G, 791.695301, CD, Mloid, CD.Mac.CXCL3.APOC1;
CD.Mloid.macrophage_chemokine.APOE_APOC1_CD163.VSIG4_CLEC10A_C1QC, 1500.162153, CD, Mloid, CD.Mac.CXCL2.TNF;
CD.Mloid.macrophage_chemokine.dendritic_CLEC10A_CD1C_AREG_CSF2RA_IL23A, 530.1973044, CD, Mloid, CD.Mac/DC.CXCL8.NLRP3;
CD.Mloid.macrophage_chemokine.doublets_plasma, 65.28110956, CD, Doublets/Mloid, CD.Doublets/Mac.IGHA1.JCHAIN; CD.Mloid.macrophage_chemokine.FCN1_AREG,
1161.620702, CD, Mloid, CD.Mono.CXCL3.FCN1; CD.Mloid.macrophage_chemokine.LGMN_FOLR2_MAF_AXL, 4992.115821, CD, Mloid, CD.Mac.FOLR2.LILRB5;
CD.Mloid.macrophage_chemokine.mitochondrial.CD74_C1Q_APOE, 324.9579509, CD, Mloid, CD.Mac.CXCL8.HES1;
CD.Mloid.macrophage_chemokine.mitochondrial.dendritic_CD1C_CD1E_S1PR4, 88.84486529, CD, Mloid, CD.Mono/Mac/DC.CXCL8.CLEC10A;
CD.Mloid.macrophage_chemokine.mitochondrial.doublets_plasma, 499.2950147, CD, Doublets/Mloid, CD.Doublets/DC.CCL4L2.IL1B;
CD.Mloid.macrophage_chemokine.mitochondrial.FOS_JUN_KLF4, 506.5752768, CD, Mloid, CD.Mac.JUN.EGR1;
CD.Mloid.macrophage_chemokine.mitochondrial.SOD2_FCN1, 328.7419931, CD, Mloid, CD.Mono/Mac.CCL4.DDX3Y;
CD.Mloid.macrophage_chemokine.S100A8_S100A9_CXCL9_CXCL10_TNF_inflamonocyte, 5394.319857, CD, Mloid, CD.Mono.CXCL10.TNF;
CD.Mloid.macrophage_chemokine.SEPP1_C1Q, 343.5765579, CD, Mloid, CD.Mac.SEPP1.CXCL3; CD.Mloid.macrophage_CXCL9_CXCL10_IDO1_STAT1, 841.9481997, CD,
Mloid, CD.Mac/DC.CXCL10.CCL19; CD.Mloid.macrophage_CXCL9_CXCL10_IDO1_STAT1.APOE_APOC1_ADAMDEC1, 1744.76063, CD, Mloid, CD.Mac/DC.CXCL10.CLEC4E;
CD.Mloid.macrophage_CXCL9_CXCL10_IDO1_STAT1.CD1E_P2RY14_P2RY13, 741.6153812, CD, Mloid, CD.DC.CXCL10.IDO1;
CD.Mloid.macrophage_CXCL9_CXCL10_IDO1_STAT1.doublet_bcll, 1244.503931, CD, Mloid, CD.Mono/Mac.CXCL10.GBP1;
CD.Mloid.macrophage_CXCL9_CXCL10_IDO1_STAT1.HSP, 323.1060191, CD, Mloid, CD.Mono/Mac/DC.CXCL10.IFIT2;
CD.Mloid.macrophage_CXCL9_CXCL10_IDO1_STAT1.macrophage_chemokine_TNF_CD163, 2176.044607, CD, Mloid, CD.Mono/Mac.CXCL10.CXCL11;
CD.Mloid.macrophage_CXCL9_CXCL10_IDO1_STAT1.NRG1_LYZ, 227.8834711, CD, Mloid, CD.Mono/Mac.CXCL10.LYZ;
CD.Mloid.macrophage_CXCL9_CXCL10_IDO1_STAT1.S100A8_S100A9_S100A4_FCN1_LILRA5, 1706.207989, CD, Mloid, CD.Mono/Mac.CXCL10.FCN1;
CD.Mloid.mitochondrial, 1078.75219, CD, LowQual/Mloid, CD.Mito.MT-CO2.MT-ND2; CD.Mloid.plasmacytoid_DC_IRF7_IL3RA, 1.66E+302, CD, Mloid, CD.pDC.IRF7.IL3RA;
CD.Mloid.proliferating, 84633.2359, CD, Mloid, CD.Mac/cDC1.MKI67.IDO1; CD.Mloid.proliferating.CQ1A_CQ1C_macrophage, 26205.64722, CD, Mloid,
CD.Mac.MKI67.APOE; CD.Mloid.proliferating.dendritic_CLEC10A_CD1C_FCER1A_CD207, 30743.62591, CD, Mloid, CD.cDC2.MKI67.CD1C;
CD.Mloid.proliferating.IG_enriched, 2355.031452, CD, Mloid, CD.Myeloid. MKI67.IGKC; CD.Mloid.proliferating.TMSB4X_ribosomal, 5160.118558, CD, Mloid,
CD.Myeloid.MKI67.PCNA; CD.Mloid.ribosomal.dendritic_CLEC10A_CD1C, 49.82933041, CD, Doublets/Mloid, CD.Doublets/cDC2.IGKC.CLEC10A;
CD.Mloid.ribosomal.dendritic_CLEC9A_IRF8_IDO1_lowBATF3, 1260.79923, CD, Mloid, CD.cDC1.CLEC9A.IDO1; CD.Mloid.ribosomal.doublet_mito_IgH_epithelial,
578.8119296, CD, Doublets/Mloid, CD.Doublets/Mloid.IGHA1.REG1A; CD.Mloid.ribosomal.doublets_epithelial, 10192.0858, CD, Doublets/Mloid,
CD.Doublets/Mloid.DEFA5.DEFA6; CD.Mloid.ribosomal.IL1B_SOD2_LYZ.HLADR_CD74_LYZ, 0, CD, Doublets/Mloid, CD.Doublets/Mloid.CD74;
CD.Mloid.ribosomal.IL1B_SOD2_LYZ.PRDX1_FTL_LCP1, 8.157640985, CD, Doublets/Mloid, CD.Doublets/Mloid.JCHAIN; CD.Mloid.ribosomal.IL1B_SOD2_LYZ.ribosomal,
37.60154543, CD, Doublets/Mloid, CD.Doublets/Mloid.REG1A.IGHA1; CD.Mloid.ribosomal.lower_quality, 9.770829062, CD, Doublets/Mloid,
CD.Doublets/Mloid.LGALS2.DEFA6; CD.Mloid.ribosomal.ribosomal, 20.22351234, CD, Doublets/Mloid, CD.Doublets/Mloid.RPL31.RPS21; CD.Mloid.ribosomal.SEPP1_C1Q,
43.0156189, CD, Doublets/Mloid, CD.Doublets/Mac.IGHA1.DEFA5; CD.Mloid.S100A8_S100A9_inflamonocyte, 29095.59737, CD, Mloid, CD.Mono.S100A8.S100A9;
CD.Mstcl.AREG_CPA3_GPR65, 299.3756193, CD, Mstcl, CD.Mstcl.AREG.ADCYAP1; CD.Mstcl.doublets_Bclls_myeloid, 1438.960969, CD, Doublets/Mstcl,
CD.Doublets/Mstcl.HLA-DQB1.HLA-DPA1; CD.Mstcl.doublets_epithelial, 289.5711762, CD, Doublets/Mstcl, CD.Doublets/Mstcl.PHGR1.KRT18;
CD.Mstcl.doublets_Tclls.CD74_IFI44L, 831.2721764, CD, Mstcl, CD.Mstcl.TRBC1.IFI44L; CD.Mstcl.doublets_Tclls.EEF_RPL, 2203.026502, CD, Mstcl, CD.Mstcl.CD3D.CD2;
CD.Mstcl.doublets_Tclls.TPSB2_CD69, 1675.122943, CD, Mstcl, CD.Mstcl.KLRB1.GNLY; CD.Mstcl.GATA2_HEXIM1.doublets_epithelial, 92.88518767, CD, Doublets/Mstcl,
CD.Doublets/Mstcl.FOS.JUN; CD.Mstcl.GATA2_HEXIM1.NUCB2_MANF, 134.2997712, CD, Mstcl, CD.Mstcl.NUCB2.HSPA5; CD.Mstcl.GATA2_HEXIM1.TPSB2_TPSAB1,
277.4291758, CD, Mstcl, CD.Mstcl.FOS.JUN; CD.Mstcl.HSP_high, 367.0670227, CD, Mstcl, CD.Mstcl.HSPA1A.HSP90AA1; CD.Mstcl.plasmablasts, 568.0556419, CD, Mstcl,
CD.Mstcl.JCHAIN.IGKC; CD.Mstcl.proliferating_Mastcells, 73371.26595, CD, Mstcl, CD.Mstcl.CDK1.KIAA0101; CD.Mstcl.ribosomal, 318.870581, CD, Mstcl,
CD.Mstcl.DEFA5.RPLP2; CD.Mstcl.TCL1A_AICDA_class_switched_Bcells, 14127.993, CD, Mstcl, CD.Mstcl.TCL1A.TK1; CD.Mstcl.TPSAB1_CTSG_CMA1, 173.3608487, CD,
Mstcl, CD.Mstcl.CTSG.CD69; CD.Tclls.CCR7_JUNB_CXCR4, 435.0597194, CD, Tclls, CD.T.CCR7.GPR183; CD.Tclls.cytotoxic_GNLY_NKG7_FCER1G_HOPX.doublet_bcll,
10803.89892, CD, Doublets/Tclls, CD.Doublets/T.BANK1.CD79A; CD.Tclls.cytotoxic_GNLY_NKG7_FCER1G_HOPX.doublets_epithelial, 15415.56429, CD, Doublets/Tclls,
CD.Doublets/T.GNLY.KLHL23; CD.Tclls.cytotoxic_GNLY_NKG7_FCER1G_HOPX.FGFBP2_FCGR3A_CX3CR1_PRF1.CD3D_IL32, 36148.49095, CD, Tclls, CD.T.GNLY.GZMH;
CD.Tclls.cytotoxic_GNLY_NKG7_FCER1G_HOPX.FGFBP2_FCGR3A_CX3CR1_PRF1.FCER1G_TYROBP_KLRB1, 53877.88262, CD, Tclls, CD.NK.GNLY.GZMB;
CD.Tclls.cytotoxic_GNLY_NKG7_FCER1G_HOPX.FGFBP2_FCGR3A_CX3CR1_PRF1.HSP, 21315.64366, CD, Tclls, CD.NK.GNLY.FCGR3A;
CD.Tclls.cytotoxic_GNLY_NKG7_FCER1G_HOPX.HSP, 8622.73046, CD, Tclls, CD.T.GNLY.HOPX;
CD.Tclls.cytotoxic_GNLY_NKG7_FCER1G_HOPX.innate_GZMA_KLRB1_FCER1G_AREG_TYROBP, 16273.23381, CD, Tclls, CD.NK.GNLY.FCER1G;
CD.Tclls.cytotoxic_GNLY_NKG7_FCER1G_HOPX.innate_SELL_KLRC1_AREG, 14624.80356, CD, Tclls, CD.NK.GNLY.KLRC1;
CD.Tclls.cytotoxic_GNLY_NKG7_FCER1G_HOPX.innate_TRDC_TYROBP_XCL1, 12887.23539, CD, Tclls, CD.NK.GNLY.XCL1;
CD.Tclls.cytotoxic_GNLY_NKG7_FCER1G_HOPX.NFKB_CD69_IFNG, 13476.95242, CD, Tclls, CD.NK.GNLY.IFNG;
CD.Tclls.cytotoxic_GNLY_NKG7_FCER1G_HOPX.Tclls_CD3D_CCL20_CXCR6_IL26_TRAC_IFNG, 11079.38738, CD, Tclls, CD.T.GNLY.CSF2;
CD.Tclls.cytotoxic_GNLY_NKG7_FCER1G_HOPX.Tclls_CD8A_CDB_TRAC_IL32_CD27, 9446.899769, CD, Tclls, CD.T.GNLY.CTSW;
CD.Tclls.cytotoxic_GNLY_NKG7_FCER1G_HOPX.Tclls_IL32_TRAC_LAG3_CD69_IFNG, 8178.545933, CD, Tclls, CD.T.GNLY.IFNG;
CD.Tclls.cytotoxic_GNLY_NKG7_FCER1G_HOPX.Tclls_mitochondrial, 8039.721173, CD, Tclls, CD.T.GNLY.MT-CO2;
CD.Tclls.cytotoxic_GNLY_NKG7_FCER1G_HOPX.Tclls_ribosomal, 5893.754126, CD, Tclls, CD.T.GNLY.IGLC2; CD.Tclls.cytotoxic_GZM_NKG7_CCL5, 5383.429489, CD, Tclls,
CD.T.CCL4.GMZK; CD.Tclls.cytotoxic_IEL_FCER1G_NKG7_TYROBP_CD160_AREG, 9393.948759, CD, Tclls, CD.NK.CCL3.CD160; CD.Tclls.doublet_bcll, 20420.33869, CD,
Doublets/Tclls, CD.Doublets/T.VPREB3.MS4A1; CD.Tclls.doublets_plasma_epi_patient14, 19774.37285, CD, Doublets/Tclls, CD.Doublets/T.APOA1.APOA4;
CD.Tclls.FOS_JUN_activated_NFKB.CCL20_LTB_IFNG_CSF2, 10895.08307, CD, Tclls, CD.T.CCL20.IFNG;
CD.Tclls. FOS_JUN_activated_NFKB.FOS_JUN_EGR1_CCL5.doublets_epithelial, 1539.342734, CD, Doublets/Tclls, CD.Doublets/T.APOA1.EGR4;
CD.Tclls.FOS_JUN_activated_NFKB.FOS_JUN_EGR1_CCL5.NKG7_GZMA_TNF, 7284.469404, CD, Tclls, CD.T.EGR1.TNF;
CD. Tclls.FOS_JUN_activated_NFKB.FOS_JUN_EGR1_CCL5.TNFAIP3_KLF6, 2702.03232, CD, Tclls, CD.T.EGR1.IFNG;
CD.Tclls.FOS_JUN_activated_NFKB.IFI44L_LY6E_ISG15_IL22, 9355.831312, CD, Tclls, CD.T.CCL20.IL22; CD.Tclls.IL32_BATF_TIGIT_CTLA4_CD27.ALOX5AP_JUNB,
792.2646191, CD, Tclls, CD.T.TNFRSF4.MAF; CD.Tclls.IL32_BATF_TIGIT_CTLA4_CD27.CCR7_FOXP1_IKZF3_IL10, 959.8632197, CD, Tclls, CD.T.LAG3.BATF;
CD.Tclls.IL32_BATF_TIGIT_CTLA4_CD27.doublets_epithelial, 1737.026552, CD, Doublets/Tclls, CD.Doublets/T.DEFA5.AC1336442;
CD.Tclls.IL32_BATF_TIGIT_CTLA4_CD27.IFI44L_MX1_ISG15, 2881.780265, CD, Tclls, CD.T.IFI6.IRF7; CD.Tclls.IL32_BATF_TIGIT_CTLA4_CD27.KLRB1_PTGER4_RORA,
662.8729312, CD, Tclls, CD.T.MAF.CTLA4; CD.Tclls.IL32_BATF_TIGIT_CTLA4_CD27.mitochondrial_IL32_CTLA4, 2605.837175, CD, Tclls, CD.T.CTLA4.IL2RA;
CD.Tclls.IL32_BATF_TIGIT_CTLA4_CD27.mitochondrial.CXCR4_NR3C1, 265.5496026, CD, Tclls, CD.T.NR3C1.CCR6;
CD.Tclls.IL32_BATF_TIGIT_CTLA4_CD27.mitochondrial.doublets_plasma, 148.5315372, CD, Doublets/Tclls, CD.Doublets/T.IGKC.JCHAIN;
CD.Tclls.IL32_BATF_TIGIT_CTLA4_CD27.mitochondrial.TIGIT_ICOS, 1257.003192, CD, Tclls, CD.T.TIGIT.CTLA4; CD.Tclls.IL32_BATF_TIGIT_CTLA4_CD27.ribosomal,
1186.583243, CD, Tclls, CD.T.CARD16.GB2; CD.Tclls.IL32_BATF_TIGIT_CTLA4_CD27.TNFRSF18_TNFRSF4_IL2RA_CTLA4_BATF, 8957.730377, CD, Tclls,
CD.T.TNFRSF18.FOXP3; CD.Tclls.IL7R_ANXA1_patient11_patient16.CD28_CCR6_ICOS, 1333.660772, CD, LowQual/Tclls, CD.T.ANXA1.IL7R;
CD.Tclls.IL7R_ANXA1_patient11_patient16.CD8A_CD8B_CCL5, 823.6150663, CD, LowQual/Tclls, CD.T.PDE3B.IL7R;
CD.Tclls.IL7R_ANXA1_patient11_patient16.doublets_plasma_patient16.CD8A_CD8B_CCL5_CD160_HOPX, 237.9461142, CD, LowQual/Tclls, CD.T.CD8A.CCL5;
CD.Tclls.IL7R_ANXA1_patient11_patient16.doublets_plasma_patient16.GNLY_NKG7_NCR3, 309.6012407, CD, LowQual/Tclls, CD.T.NCR3.KLRB1;
CD.Tclls.IL7R_ANXA1_patient11_patient16.doublets_plasma_patient16.LTB_CD2_CCR6, 576.2492252, CD, Doublets/Tclls, CD.Doublets/T.IGHA2.JCHAIN;
CD.Tclls.IL7R_ANXA1_patient11_patient16.doublets_plasma_patient16.MALAT1_JUNB, 416.5376645, CD, Doublets/Tclls, CD.Doublets/T.JCHAIN.IGHA2;
CD.Tclls.IL7R_ANXA1_patient11_patient16.HSP, 3837.262795, CD, LowQual/Tclls, CD.T.HSPA1A.HSP90AA1; CD.Tclls.IL7R_ANXA1_patient11_patient16.HSP_XIST,
949.465737, CD, LowQual/Tclls, CD.T.HSPA1B.HSPA1A; CD.Tclls.IL7R_ANXA1_patient11_patient16.TRDC_TRGC2_SOX4_GZMK, 861.2671534, CD, LowQual/Tclls,
CD.T.TRDC.SOX4; CD.Tclls.KLRB1_IL7R_CCL5_CD3D_CD69.CD69_activated_FOS_JUN, 1197.724883, CD, Tclls, CD.T.IFI44L.PTGER4;
CD.Tclls.KLRB1_IL7R_CCL5_CD3D_CD69.CD8A_CD8B_RUNX3_CD160, 2630.817707, CD, Tclls, CD.T.CD8B.TRGC2;
CD.Tclls.KLRB1_IL7R_CCL5_CD3D_CD69.doublets_epithelial.CD8A_CD8B_CCL5, 687.5188334, CD, Doublets/Tclls, CD.Doublets/T.CD8B.DEFA6;
CD.Tclls.KLRB1_IL7R_CCL5_CD3D_CD69.doublets_epithelial.FABP5_ALOX5AP_GZM, 623.841958, CD, Doublets/Tclls, CD.Doublets/T.LGALS3.DEFA6;
CD.Tclls.KLRB1_IL7R_CCL5_CD3D_CD69.doublets_epithelial.FOS_JUN_activated_KLF6, 684.5033338, CD, Doublets/Tclls, CD.Doublets/T.DEFA5.DEFA6;
CD.Tclls.KLRB1_IL7R_CCL5_CD3D_CD69.doublets_epithelial.KLRB1_TNFRSF13B_ICOS, 501.9464596, CD, Doublets/Tclls, CD.Doublets/T.KLRB1.DEFA5;
CD.Tclls.KLRB1_IL7R_CCL5_CD3D_CD69.doublets_plasma, 177.5401766, CD, Doublets/Tclls, CD.Doublets/T.ANXA1.IGKC;
CD.Tclls.KLRB1_IL7R_CCL5_CD3D_CD69.GZMA_IFNG, 767.5666072, CD, Tclls, CD.T.CCL20.RORA; CD.Tclls.KLRB1_IL7R_CCL5_CD3D_CD69.KLRB1_LTB, 991.4616015, CD,
Tclls, CD.T.KLRB1.LTF; CD.Tclls.KLRB1_IL7R_CCL5_CD3D_CD69.TRDC_TRGC2_SOX4, 984.4436261, CD, Tclls, CD.T.TRDC.TRGC2; CD.Tclls.mitochondrial.doublet_bcll,
2907.708502, CD, Doublets/Tclls, CD.Doublets/T.MS4A1.MT-ND4L; CD.Tclls.mitochondrial.doublets_epithelial, 2965.142853, CD, Doublets/Tclls,
CD.Doublets/T.APOA1.APOA4; CD.Tclls.mitochondrial.ribosomal, 611.3895344, CD, Tclls, CD.T.MT-CO2.MT-CO1; CD.Tclls.mitochondrial.ribosomal_KLRB1, 745.2051821,
CD, Tclls, CD.T.MT-CO2.MT-CO3; CD.Tclls.mitochondrial.TRAC_CD3D_CXCR4.CCL5_CD8A, 63.12983283, CD, Tclls, CD.T.MT-CO2.PTGER4;
CD.Tclls.mitochondrial.TRAC_CD3D_CXCR4.CCR7_CXCR4, 90.99002378, CD, Tclls, CD.T.MT-CO2.CCR7; CD.Tclls.mitochondrial.TRAC_CD3D_CXCR4.CD69_GZMH,
50.89909245, CD, Tclls, CD.T.GZMA.JAML; CD.Tclls.mitochondrial.TRAC_CD3D_CXCR4.CTLA4_TIGIT_IL2RA_TNFRSF18, 157.4728677, CD, Tclls, CD.T.CTLA4.CD27;
CD.Tclls.mitochondrial.TRAC_CD3D_CXCR4.doublets_plasma, 181.3465159, CD, Doublets/Tclls, CD.Doublets/T.JCHAIN.IGKC;
CD.Tclls.mitochondrial.TRAC_CD3D_CXCR4.ribosomal, 29.72062171, CD, Tclls, CD.T.B2M.MT-CO2; CD.Tclls.proliferating.CD27_IL2RA_CTLA4_BATF_TNFSRF4,
37495.44917, CD, Tclls, CD.T.MKI67.FOXP3; CD.Tclls.proliferating.FABP5_S100A4_HOPX, 37195.86915, CD, Tclls, CD.T.MKI67.IFNG;
CD.Tclls.proliferating.IFNG_IL22_CCL20, 69537.99537, CD, Tclls, CD.T.MKI67.IL22; CD.Tclls.proliferating.mitochondrial, 19103.10292, CD, Tclls, CD.T.MKI67.MT-CO2;
CD.Tclls.proliferating.ribosomal, 14219.05349, CD, Tclls, CD.T.MKI67.NKG7; CD.Tclls.proliferating.ribosomal.CD27_GZMK, 219357.6967, CD, Tclls, CD.T.MKI67.CD38;
CD.Tclls.proliferating.ribosomal.CXCR3_GPR171, 29132.50566, CD, Tclls, CD.T.MKI67.ITGB7; CD.Tclls.proliferating.ribosomal.HSP, 20166.41105, CD, Tclls,
CD.T.MKI67.RRM2; CD.Tclls.proliferating.ribosomal.proliferating, 36179.26211, CD, Tclls, CD.T.MKI67.MAF; CD.Tclls.proliferating.ribosomal.ribosomal, 10707.35323, CD,
Tclls, CD.T.MKI67.CCR7; CD.Tclls.proliferating.TYROBP_FCER1G_AREG_NKG7_GNLY, 54445.17066, CD, Tclls, CD.NK.MKI67.GZMA; CD.Tclls.ribosomal, 1806.804214, CD,
Tclls, CD.T.CCR7.SELL; CD.Tclls.Type17_ILC_LST1_TYROBP_AREG_KIT.AHR_CD164_STAT1, 21989.39314, CD, Tclls, CD.ILC.AREG.AHR;
CD.Tclls.Type17_ILC_LST1_TYROBP_AREG_KIT.doublets_plasma, 3124.098212, CD, Doublets/Tclls, CD.Doublets/T.KRT86.KIT;
CD.Tclls.Type17_ILC_LST1_TYROBP_AREG_KIT.IFI44L_LY6E_MX1, 42282.3875, CD, Tclls, CD.ILC.KRT81.IL22;
CD.Tclls.Type17_ILC_LST1_TYROBP_AREG_KIT.KLRC1_KLF2_IL22, 37796.4733, CD, Tclls, CD.ILC.IL22.KIT; CD.Tclls.Type17_ILC_LST1_TYROBP_AREG_KIT.ribosomal,
19539.08011, CD, Tclls, CD.ILC.LST1.AREG;

TABLE 10
Number of cells per patient per end cell cluster. Table organized by patient number, subset short name, Frequency; patient number, subset short name, Frequency; etc.

p011, CD.B.ACTB.JCHAIN, 20; p014, CD.B.ACTB.JCHAIN, 37; p016, CD.B.ACTB.JCHAIN, 71; p018, CD.B.ACTB.JCHAIN, 11; p019, CD.B.ACTB.JCHAIN, 136; p022,
CD.B.ACTB.JCHAIN, 12; p024, CD.B.ACTB.JCHAIN, 6; p026, CD.B.ACTB.JCHAIN, 85; p027, CD.B.ACTB.JCHAIN, 288; p031, CD.B.ACTB.JCHAIN, 44; p032, CD.B.ACTB.JCHAIN,
41; p041, CD.B.ACTB.JCHAIN, 32; p042, CD.B.ACTB.JCHAIN, 63; p048, CD.B.ACTB.JCHAIN, 85; p011, CD.B.FCRL4.OSTN-AS1, 3; p014, CD.B.FCRL4.OSTN-AS1, 1; p016,
CD.B.FCRL4.OSTN-AS1, 10; p018, CD.B.FCRL4.OSTN-AS1, 29; p019, CD.B.FCRL4.OSTN-AS1, 46; p022, CD.B.FCRL4.OSTN-AS1, 10; p024, CD.B.FCRL4.OSTN-AS1, 6; p026,
CD.B.FCRL4.OSTN-AS1, 8; p027, CD.B.FCRL4.OSTN-AS1, 49; p031, CD.B.FCRL4.OSTN-AS1, 91; p032, CD.B.FCRL4.OSTN-AS1, 26; p041, CD.B.FCRL4.OSTN-AS1, 16; p042,
CD.B.FCRL4.OSTN-AS1, 26; p048, CD.B.FCRL4.OSTN-AS1, 27; p011, CD.B.HCK.DUSP4, 1; p014, CD.B.HCK.DUSP4, 0; p016, CD.B.HCK.DUSP4, 0; p018, CD.B.HCK.DUSP4, 0;
p019, CD.B.HCK.DUSP4, 0; p022, CD.B.HCK.DUSP4, 0; p024, CD.B.HCK.DUSP4, 1; p026, CD.B.HCK.DUSP4, 348; p027, CD.B.HCK.DUSP4, 1; p031, CD.B.HCK.DUSP4, 1; p032,
CD.B.HCK.DUSP4, 0; p041, CD.B.HCK.DUSP4, 1; p042, CD.B.HCK.DUSP4, 0; p048, CD.B.HCK.DUSP4, 0; p011, CD.B.IFI44L.IGLC2, 0; p014, CD.B.IFI44L.IGLC2, 0; p016,
CD.B.IFI44L.IGLC2, 0; p018, CD.B.IFI44L.IGLC2, 0; p019, CD.B.IFI44L.IGLC2, 2; p022, CD.B.IFI44L.IGLC2, 0; p024, CD.B.IFI44L.IGLC2, 0; p026, CD.B.IFI44L.IGLC2, 167; p027,
CD.B.IFI44L.IGLC2, 4; p031, CD.B.IFI44L.IGLC2, 3; p032, CD.B.IFI44L.IGLC2, 4; p041, CD.B.IFI44L.IGLC2, 1; p042, CD.B.IFI44L.IGLC2, 0; p048, CD.B.IFI44L.IGLC2, 0; p011,
CD.B.IFI44L.ISG15, 0; p014, CD.B.IFI44L.ISG15, 0; p016, CD.B.IFI44L.ISG15, 2; p018, CD.B.IFI44L.ISG15, 0; p019, CD.B.IFI44L.ISG15, 28; p022, CD.B.IFI44L.ISG15, 3; p024,
CD.B.IFI44L.ISG15, 0; p026, CD.B.IFI44L.ISG15, 331; p027, CD.B.IFI44L.ISG15, 18; p031, CD.B.IFI44L.ISG15, 8; p032, CD.B.IFI44L.ISG15, 1; p041, CD.B.IFI44L.ISG15, 3;
p042, CD.B.IFI44L.ISG15, 1; p048, CD.B.IFI44L.ISG15, 0; p011, CD.B.IGHA2.JCHAIN, 1; p014, CD.B.IGHA2.JCHAIN, 0; p016, CD.B.IGHA2.JCHAIN, 1480; p018,
CD.B.IGHA2.JCHAIN, 1; p019, CD.B.IGHA2.JCHAIN, 2; p022, CD.B.IGHA2.JCHAIN, 8; p024, CD.B.IGHA2.JCHAIN, 13; p026, CD.B.IGHA2.JCHAIN, 1; p027, CD.B.IGHA2.JCHAIN,
5; p031, CD.B.IGHA2.JCHAIN, 6; p032, CD.B.IGHA2.JCHAIN, 2; p041, CD.B.IGHA2.JCHAIN, 0; p042, CD.B.IGHA2.JCHAIN, 2; p048, CD.B.IGHA2.JCHAIN, 15; p011,
CD.B.IGHG1.IGLC2, 1; p014, CD.B.IGHG1.IGLC2, 1; p016, CD.B.IGHG1.IGLC2, 1; p018, CD.B.IGHG1.IGLC2, 3; p019, CD.B.IGHG1.IGLC2, 5; p022, CD.B.IGHG1.IGLC2, 786; p024,
CD.B.IGHG1.IGLC2, 24; p026, CD.B.IGHG1.IGLC2, 2; p027, CD.B.IGHG1.IGLC2, 11; p031, CD.B.IGHG1.IGLC2, 15; p032, CD.B.IGHG1.IGLC2, 45; p041, CD.B.IGHG1.IGLC2, 38;
p042, CD.B.IGHG1.IGLC2, 8; p048, CD.B.IGHG1.IGLC2, 8; p011, CD.B.IGLC2.HLA-DRB1, 5; p014, CD.B.IGLC2.HLA-DRB1, 2; p016, CD.B.IGLC2.HLA-DRB1, 3; p018,
CD.B.IGLC2.HLA-DRB1, 21; p019, CD.B.IGLC2.HLA-DRB1, 48; p022, CD.B.IGLC2.HLA-DRB1, 38; p024, CD.B.IGLC2.HLA-DRB1, 20; p026, CD.B.IGLC2.HLA-DRB1, 9; p027,
CD.B.IGLC2.HLA-DRB1, 172; p031, CD.B.IGLC2.HLA-DRB1, 35; p032, CD.B.IGLC2.HLA-DRB1, 86; p041, CD.B.IGLC2.HLA-DRB1, 71; p042, CD.B.IGLC2.HLA-DRB1, 36; p048,
CD.B.IGLC2.HLA-DRB1, 46; p011, CD.B.IGLC3.HLA-DRB1, 5; p014, CD.B.IGLC3.HLA-DRB1, 0; p016, CD.B.IGLC3.HLA-DRB1, 1; p018, CD.B.IGLC3.HLA-DRB1, 0; p019,
CD.B.IGLC3.HLA-DRB1, 6; p022, CD.B.IGLC3.HLA-DRB1, 0; p024, CD.B.IGLC3.HLA-DRB1, 1; p026, CD.B.IGLC3.HLA-DRB1, 1; p027, CD.B.IGLC3.HLA-DRB1, 124; p031,
CD.B.IGLC3.HLA-DRB1, 59; p032, CD.B.IGLC3.HLA-DRB1, 7; p041, CD.B.IGLC3.HLA-DRB1, 3; p042, CD.B.IGLC3.HLA-DRB1, 8; p048, CD.B.IGLC3.HLA-DRB1, 7; p011,
CD.B.IGLC3.IFI44L, 0; p014, CD.B.IGLC3.IFI44L, 0; p016, CD.B.IGLC3.IFI44L, 0; p018, CD.B.IGLC3.IFI44L, 1; p019, CD.B.IGLC3.IFI44L, 0; p022, CD.B.IGLC3.IFI44L, 1; p024,
CD.B.IGLC3.IFI44L, 0; p026, CD.B.IGLC3.IFI44L, 93; p027, CD.B.IGLC3.IFI44L, 1; p031, CD.B.IGLC3.IFI44L, 0; p032, CD.B.IGLC3.IFI44L, 1; p041, CD.B.IGLC3.IFI44L, 0; p042,
CD.B.IGLC3.IFI44L, 0; p048, CD.B.IGLC3.IFI44L, 0; p011, CD.B.IGLC3.IGLC2, 0; p014, CD.B.IGLC3.IGLC2, 3; p016, CD.B.IGLC3.IGLC2, 1; p018, CD.B.IGLC3.IGLC2, 0; p019,
CD.B.IGLC3.IGLC2, 2; p022, CD.B.IGLC3.IGLC2, 7; p024, CD.B.IGLC3.IGLC2, 0; p026, CD.B.IGLC3.IGLC2, 5; p027, CD.B.IGLC3.IGLC2, 9; p031, CD.B.IGLC3.IGLC2, 21; p032,
CD.B.IGLC3.IGLC2, 592; p041, CD.B.IGLC3.IGLC2, 10; p042, CD.B.IGLC3.IGLC2, 2; p048, CD.B.IGLC3.IGLC2, 4; p011, CD.B.IGLC3.JCHAIN, 11; p014, CD.B.IGLC3.JCHAIN, 1;
p016, CD.B.IGLC3.JCHAIN, 3; p018, CD.B.IGLC3.JCHAIN, 8; p019, CD.B.IGLC3.JCHAIN, 17; p022, CD.B.IGLC3.JCHAIN, 2; p024, CD.B.IGLC3.JCHAIN, 9; p026,
CD.B.IGLC3.JCHAIN, 7; p027, CD.B.IGLC3.JCHAIN, 72; p031, CD.B.IGLC3.JCHAIN, 34; p032, CD.B.IGLC3.JCHAIN, 15; p041, CD.B.IGLC3.JCHAIN, 18; p042, CD.B.IGLC3.JCHAIN,
129; p048, CD.B.IGLC3.JCHAIN, 49; p011, CD.B.IGLC3.RP11-347P51, 1; p014, CD.B.IGLC3.RP11-347P51, 0; p016, CD.B.IGLC3.RP11-347P51, 0; p018, CD.B.IGLC3.RP11-
347P51, 4; p019, CD.B.IGLC3.RP11-347P51, 212; p022, CD.B.IGLC3.RP11-347P51, 0; p024, CD.B.IGLC3.RP11-347P51, 0; p026, CD.B.IGLC3.RP11-347P51, 0; p027,
CD.B.IGLC3.RP11-347P51, 7; p031, CD.B.IGLC3.RP11-347P51, 0; p032, CD.B.IGLC3.RP11-347P51, 1; p041, CD.B.IGLC3.RP11-347P51, 2; p042, CD.B.IGLC3.RP11-347P51,
3; p048, CD.B.IGLC3.RP11-347P51, 2; p011, CD.B.MKI67/GC.RRM2, 0; p014, CD.B.MKI67/GC.RRM2, 0; p016, CD.B.MKI67/GC.RRM2, 0; p018, CD.B.MKI67/GC.RRM2, 0;
p019, CD.B.MKI67/GC.RRM2, 17; p022, CD.B.MKI67/GC.RRM2, 0; p024, CD.B.MKI67/GC.RRM2, 0; p026, CD.B.MKI67/GC.RRM2, 0; p027, CD.B.MKI67/GC.RRM2, 0; p031,
CD.B.MKI67/GC.RRM2, 0; p032, CD.B.MKI67/GC.RRM2, 0; p041, CD.B.MKI67/GC.RRM2, 0; p042, CD.B.MKI67/GC.RRM2, 0; p048, CD.B.MKI67/GC.RRM2, 0; p011, CD.B.MT-
CO2.MT-CO1, 93; p014, CD.B.MT-CO2.MT-CO1, 9; p016, CD.B.MT-CO2.MT-CO1, 10; p018, CD.B.MT-CO2.MT-CO1, 32; p019, CD.B.MT-CO2.MT-CO1, 98; p022, CD.B.MT-
CO2.MT-CO1, 74; p024, CD.B.MT-CO2.MT-CO1, 28; p026, CD.B.MT-CO2.MT-CO1, 67; p027, CD.B.MT-CO2.MT-CO1, 226; p031, CD.B.MT-CO2.MT-CO1, 1272; p032, CD.B.MT-
CO2.MT-CO1, 44; p041, CD.B.MT-CO2.MT-CO1, 23; p042, CD.B.MT-CO2.MT-CO1, 23; p048, CD.B.MT-CO2.MT-CO1, 73; p011, CD.B.MT-ND4L.IGHG1, 3; p014, CD.B.MT-
ND4L.IGHG1, 11; p016, CD.B.MT-ND4L.IGHG1, 0; p018, CD.B.MT-ND4L.IGHG1, 0; p019, CD.B.MT-ND4L.IGHG1, 2; p022, CD.B.MT-ND4L.IGHG1, 50; p024, CD.B.MT-
ND4L.IGHG1, 7; p026, CD.B.MT-ND4L.IGHG1, 3; p027, CD.B.MT-ND4L.IGHG1, 25; p031, CD.B.MT-ND4L.IGHG1, 18; p032, CD.B.MT-ND4L.IGHG1, 2592; p041, CD.B.MT-
ND4L.IGHG1, 21; p042, CD.B.MT-ND4L.IGHG1, 3; p048, CD.B.MT-ND4L.IGHG1, 0; p011, CD.B.RPS10.FCER2, 14; p014, CD.B.RPS10.FCER2, 0; p016, CD.B.RPS10.FCER2, 1;
p018, CD.B.RPS10.FCER2, 17; p019, CD.B.RPS10.FCER2, 2102; p022, CD.B.RPS10.FCER2, 41; p024, CD.B.RPS10.FCER2, 7; p026, CD.B.RPS10.FCER2, 24; p027,
CD.B.RPS10.FCER2, 504; p031, CD.B.RPS10.FCER2, 33; p032, CD.B.RPS10.FCER2, 19; p041, CD.B.RPS10.FCER2, 91; p042, CD.B.RPS10.FCER2, 142; p048,
CD.B.RPS10.FCER2, 174; p011, CD.B.RPS29.RPS21, 46; p014, CD.B.RPS29.RPS21, 0; p016, CD.B.RPS29.RPS21, 11; p018, CD.B.RPS29.RPS21, 121; p019, CD.B.RPS29.RPS21,
1073; p022, CD.B.RPS29.RPS21, 178; p024, CD.B.RPS29.RPS21, 137; p026, CD.B.RPS29.RPS21, 2; p027, CD.B.RPS29.RPS21, 254; p031, CD.B.RPS29.RPS21, 30; p032,
CD.B.RPS29.RPS21, 49; p041, CD.B.RPS29.RPS21, 289; p042, CD.B.RPS29.RPS21, 740; p048, CD.B.RPS29.RPS21, 210; p011, CD.B.UBE2C.KIAA0101, 9; p014,
CD.B.UBE2C.KIAA0101, 7; p016, CD.B.UBE2C.KIAA0101, 11; p018, CD.B.UBE2C.KIAA0101, 8; p019, CD.B.UBE2C.KIAA0101, 293; p022, CD.B.UBE2C.KIAA0101, 2; p024,
CD.B.UBE2C.KIAA0101, 19; p026, CD.B.UBE2C.KIAA0101, 109; p027, CD.B.UBE2C.KIAA0101, 22; p031, CD.B.UBE2C.KIAA0101, 20; p032, CD.B.UBE2C.KIAA0101, 28; p041,
CD.B.UBE2C.KIAA0101, 13; p042, CD.B.UBE2C.KIAA0101, 59; p048, CD.B.UBE2C.KIAA0101, 16; p011, CD.B/DZ.AICDA.PLK1, 0; p014, CD.B/DZ.AICDA.PLK1, 0; p016,
CD.B/DZ.AICDA.PLK1, 0; p018, CD.B/DZ.AICDA.PLK1, 1; p019, CD.B/DZ.AICDA.PLK1, 31; p022, CD.B/DZ.AICDA.PLK1, 0; p024, CD.B/DZ.AICDA.PLK1, 4; p026,
CD.B/DZ.AICDA.PLK1, 11; p027, CD.B/DZ.AICDA.PLK1, 0; p031, CD.B/DZ.AICDA.PLK1, 0; p032, CD.B/DZ.AICDA.PLK1, 0; p041, CD.B/DZ.AICDA.PLK1, 0; p042,
CD.B/DZ.AICDA.PLK1, 11; p048, CD.B/DZ.AICDA.PLK1, 1; p011, CD.B/DZ.AICDA/CDK1, 0; p014, CD.B/DZ.AICDA/CDK1, 0; p016, CD.B/DZ.AICDA/CDK1, 0; p018,
CD.B/DZ.AICDA/CDK1, 0; p019, CD.B/DZ.AICDA/CDK1, 0; p022, CD.B/DZ.AICDA/CDK1, 0; p024, CD.B/DZ.AICDA/CDK1, 7; p026, CD.B/DZ.AICDA/CDK1, 3; p027,
CD.B/DZ.AICDA/CDK1, 0; p031, CD.B/DZ.AICDA/CDK1, 1; p032, CD.B/DZ.AICDA/CDK1, 0; p041, CD.B/DZ.AICDA/CDK1, 0; p042, CD.B/DZ.AICDA/CDK1, 17; p048,
CD.B/DZ.AICDA/CDK1, 0; p011, CD.B/DZ.HIST1H1B.MKI67, 0; p014, CD.B/DZ.HIST1H1B.MKI67, 0; p016, CD.B/DZ.HIST1H1B.MKI67, 0; p018, CD.B/DZ.HIST1H1B.MKI67,
1; p019, CD.B/DZ.HIST1H1B.MKI67, 0; p022, CD.B/DZ.HIST1H1B.MKI67, 0; p024, CD.B/DZ.HIST1H1B.MKI67, 17; p026, CD.B/DZ.HIST1H1B.MKI67, 0; p027,
CD.B/DZ.HIST1H1B.MKI67, 0; p031, CD.B/DZ.HIST1H1B.MKI67, 3; p032, CD.B/DZ.HIST1H1B.MKI67, 17; p041, CD.B/DZ.HIST1H1B.MKI67, 0; p042,
CD.B/DZ.HIST1H1B.MKI67, 2; p048, CD.B/DZ.HIST1H1B.MKI67, 2; p011, CD.B/DZ.RGS13.AICDA, 0; p014, CD.B/DZ.RGS13.AICDA, 0; p016, CD.B/DZ.RGS13.AICDA, 3; p018,
CD.B/DZ.RGS13.AICDA, 6; p019, CD.B/DZ.RGS13.AICDA, 470; p022, CD.B/DZ.RGS13.AICDA, 0; p024, CD.B/DZ.RGS13.AICDA, 36; p026, CD.B/DZ.RGS13.AICDA, 131; p027,
CD.B/DZ.RGS13.AICDA, 0; p031, CD.B/DZ.RGS13.AICDA, 4; p032, CD.B/DZ.RGS13.AICDA, 17; p041, CD.B/DZ.RGS13.AICDA, 2; p042, CD.B/DZ.RGS13.AICDA, 122; p048,
CD.B/DZ.RGS13.AICDA, 17; p011, CD.B/GC.CENPE.TOP2A, 0; p014, CD.B/GC.CENPE.TOP2A, 0; p016, CD.B/GC.CENPE.TOP2A, 0; p018, CD.B/GC.CENPE.TOP2A, 3; p019,
CD.B/GC.CENPE.TOP2A, 110; p022, CD.B/GC.CENPE.TOP2A, 0; p024, CD.B/GC.CENPE.TOP2A, 0; p026, CD.B/GC.CENPE.TOP2A, 0; p027, CD.B/GC.CENPE.TOP2A, 0; p031,
CD.B/GC.CENPE.TOP2A, 0; p032, CD.B/GC.CENPE.TOP2A, 0; p041, CD.B/GC.CENPE.TOP2A, 0; p042, CD.B/GC.CENPE.TOP2A, 0; p048, CD.B/GC.CENPE.TOP2A, 0; p011,
CD.B/GC.HIST1H1A.RRM2, 0; p014, CD.B/GC.HIST1H1A.RRM2, 0; p016, CD.B/GC.HIST1H1A.RRM2, 0; p018, CD.B/GC.HIST1H1A.RRM2, 0; p019,
CD.B/GC.HIST1H1A.RRM2, 68; p022, CD.B/GC.HIST1H1A.RRM2, 0; p024, CD.B/GC.HIST1H1A.RRM2, 0; p026, CD.B/GC.HIST1H1A.RRM2, 0; p027,
CD.B/GC.HIST1H1A.RRM2, 0; p031, CD.B/GC.HIST1H1A.RRM2, 0; p032, CD.B/GC.HIST1H1A.RRM2, 0; p041, CD.B/GC.HIST1H1A.RRM2, 0; p042,
CD.B/GC.HIST1H1A.RRM2, 0; p048, CD.B/GC.HIST1H1A.RRM2, 0; p011, CD.B/GC.KIAA0101.GINS2, 0; p014, CD.B/GC.KIAA0101.GINS2, 0; p016,
CD.B/GC.KIAA0101.GINS2, 4; p018, CD.B/GC.KIAA0101.GINS2, 4; p019, CD.B/GC.KIAA0101.GINS2, 222; p022, CD.B/GC.KIAA0101.GINS2, 0; p024,
CD.B/GC.KIAA0101.GINS2, 15; p026, CD.B/GC.KIAA0101.GINS2, 42; p027, CD.B/GC.KIAA0101.GINS2, 0; p031, CD.B/GC.KIAA0101.GINS2, 6; p032,
CD.B/GC.KIAA0101.GINS2, 6; p041, CD.B/GC.KIAA0101.GINS2, 0; p042, CD.B/GC.KIAA0101.GINS2, 66; p048, CD.B/GC.KIAA0101.GINS2, 1; p011, CD.B/GC.MKI67.RRM2,
1; p014, CD.B/GC.MKI67.RRM2, 0; p016, CD.B/GC.MKI67.RRM2, 0; p018, CD.B/GC.MKI67.RRM2, 3; p019, CD.B/GC.MKI67.RRM2, 4; p022, CD.B/GC.MKI67.RRM2, 0; p024,
CD.B/GC.MKI67.RRM2, 0; p026, CD.B/GC.MKI67.RRM2, 12; p027, CD.B/GC.MKI67.RRM2, 2; p031, CD.B/GC.MKI67.RRM2, 4; p032, CD.B/GC.MKI67.RRM2, 5; p041,
CD.B/GC.MKI67.RRM2, 2; p042, CD.B/GC.MKI67.RRM2, 0; p048, CD.B/GC.MKI67.RRM2, 2; p011, CD.B/GC.RRM2.TK1, 0; p014, CD.B/GC.RRM2.TK1, 0; p016,
CD.B/GC.RRM2.TK1, 0; p018, CD.B/GC.RRM2.TK1, 0; p019, CD.B/GC.RRM2.TK1, 0; p022, CD.B/GC.RRM2.TK1, 0; p024, CD.B/GC.RRM2.TK1, 0; p026, CD.B/GC.RRM2.TK1,
63; p027, CD.B/GC.RRM2.TK1, 0; p031, CD.B/GC.RRM2.TK1, 0; p032, CD.B/GC.RRM2.TK1, 0; p041, CD.B/GC.RRM2.TK1, 0; p042, CD.B/GC.RRM2.TK1, 0; p048,
CD.B/GC.RRM2.TK1, 0; p011, CD.B/GC.RRM2.TYMS, 0; p014, CD.B/GC.RRM2.TYMS, 0; p016, CD.B/GC.RRM2.TYMS, 0; p018, CD.B/GC.RRM2.TYMS, 2; p019,
CD.B/GC.RRM2.TYMS, 43; p022, CD.B/GC.RRM2.TYMS, 0; p024, CD.B/GC.RRM2.TYMS, 0; p026, CD.B/GC.RRM2.TYMS, 1; p027, CD.B/GC.RRM2.TYMS, 0; p031,
CD.B/GC.RRM2.TYMS, 0; p032, CD.B/GC.RRM2.TYMS, 0; p041, CD.B/GC.RRM2.TYMS, 0; p042, CD.B/GC.RRM2.TYMS, 1; p048, CD.B/GC.RRM2.TYMS, 0; p011,
CD.B/GC.RRM2.ZWINT, 0; p014, CD.B/GC.RRM2.ZWINT, 0; p016, CD.B/GC.RRM2.ZWINT, 0; p018, CD.B/GC.RRM2.ZWINT, 1; p019, CD.B/GC.RRM2.ZWINT, 1; p022,
CD.B/GC.RRM2.ZWINT, 0; p024, CD.B/GC.RRM2.ZWINT, 2; p026, CD.B/GC.RRM2.ZWINT, 0; p027, CD.B/GC.RRM2.ZWINT, 0; p031, CD.B/GC.RRM2.ZWINT, 0; p032,
CD.B/GC.RRM2.ZWINT, 0; p041, CD.B/GC.RRM2.ZWINT, 0; p042, CD.B/GC.RRM2.ZWINT, 44; p048, CD.B/GC.RRM2.ZWINT, 0; p011, CD.B/GC.TYMS.MKI67, 0; p014,
CD.B/GC.TYMS.MKI67, 0; p016, CD.B/GC.TYMS.MKI67, 0; p018, CD.B/GC.TYMS.MKI67, 0; p019, CD.B/GC.TYMS.MKI67, 59; p022, CD.B/GC.TYMS.MKI67, 0; p024,
CD.B/GC.TYMS.MKI67, 1; p026, CD.B/GC.TYMS.MKI67, 2; p027, CD.B/GC.TYMS.MKI67, 0; p031, CD.B/GC.TYMS.MKI67, 0; p032, CD.B/GC.TYMS.MKI67, 0; p041,
CD.B/GC.TYMS.MKI67, 0; p042, CD.B/GC.TYMS.MKI67, 1; p048, CD.B/GC.TYMS.MKI67, 0; p011, CD.B/LZ.CCL22.NPW, 0; p014, CD.B/LZ.CCL22.NPW, 0; p016,
CD.B/LZ.CCL22.NPW, 3; p018, CD.B/LZ.CCL22.NPW, 11; p019, CD.B/LZ.CCL22.NPW, 68; p022, CD.B/LZ.CCL22.NPW, 1; p024, CD.B/LZ.CCL22.NPW, 4; p026,
CD.B/LZ.CCL22.NPW, 5; p027, CD.B/LZ.CCL22.NPW, 8; p031, CD.B/LZ.CCL22.NPW, 2; p032, CD.B/LZ.CCL22.NPW, 6; p041, CD.B/LZ.CCL22.NPW, 5; p042,
CD.B/LZ.CCL22.NPW, 19; p048, CD.B/LZ.CCL22.NPW, 16; p011, CD.Bclls.CD69.GPR183, 110; p014, CD.Bclls.CD69.GPR183, 9; p016, CD.Bclls.CD69.GPR183, 5; p018,
CD.Bclls.CD69.GPR183, 15; p019, CD.Bclls.CD69.GPR183, 42; p022, CD.Bclls.CD69.GPR183, 96; p024, CD.Bclls.CD69.GPR183, 54; p026, CD.Bclls.CD69.GPR183, 4; p027,
CD.Bclls.CD69.GPR183, 76; p031, CD.Bclls.CD69.GPR183, 40; p032, CD.Bclls.CD69.GPR183, 821; p041, CD.Bclls.CD69.GPR183, 88; p042, CD.Bclls.CD69.GPR183, 46; p048,
CD.Bclls.CD69.GPR183, 26; p011, CD.cDC1.CLEC9A.IDO1, 52; p014, CD.cDC1.CLEC9A.IDO1, 29; p016, CD.cDC1.CLEC9A.IDO1, 29; p018, CD.cDC1.CLEC9A.IDO1, 15; p019,
CD.cDC1.CLEC9A.IDO1, 19; p022, CD.cDC1.CLEC9A.IDO1, 21; p024, CD.cDC1.CLEC9A.IDO1, 9; p026, CD.cDC1.CLEC9A.IDO1, 12; p027, CD.cDC1.CLEC9A.IDO1, 22; p031,
CD.cDC1.CLEC9A.IDO1, 68; p032, CD.cDC1.CLEC9A.IDO1, 19; p041, CD.cDC1.CLEC9A.IDO1, 12; p042, CD.cDC1.CLEC9A.IDO1, 32; p048, CD.cDC1.CLEC9A.IDO1, 20; p011,
CD.cDC1.CLEC9A.XCR1, 78; p014, CD.cDC1.CLEC9A.XCR1, 20; p016, CD.cDC1.CLEC9A.XCR1, 26; p018, CD.cDC1.CLEC9A.XCR1, 42; p019, CD.cDC1.CLEC9A.XCR1, 6; p022,
CD.cDC1.CLEC9A.XCR1, 23; p024, CD.cDC1.CLEC9A.XCR1, 38; p026, CD.cDC1.CLEC9A.XCR1, 10; p027, CD.cDC1.CLEC9A.XCR1, 16; p031, CD.cDC1.CLEC9A.XCR1, 89; p032,
CD.cDC1.CLEC9A.XCR1, 11; p041, CD.cDC1.CLEC9A.XCR1, 18; p042, CD.cDC1.CLEC9A.XCR1, 26; p048, CD.cDC1.CLEC9A.XCR1, 37; p011, CD.cDC2.CD1C.AREG, 1; p014,
CD.cDC2.CD1C.AREG, 5; p016, CD.cDC2.CD1C.AREG, 0; p018, CD.cDC2.CD1C.AREG, 1; p019, CD.cDC2.CD1C.AREG, 0; p022, CD.cDC2.CD1C.AREG, 12; p024,
CD.cDC2.CD1C.AREG, 42; p026, CD.cDC2.CD1C.AREG, 7; p027, CD.cDC2.CD1C.AREG, 6; p031, CD.cDC2.CD1C.AREG, 96; p032, CD.cDC2.CD1C.AREG, 13; p041,
CD.cDC2.CD1C.AREG, 18; p042, CD.cDC2.CD1C.AREG, 2; p048, CD.cDC2.CD1C.AREG, 0; p011, CD.cDC2.CD1C.CD207, 0; p014, CD.cDC2.CD1C.CD207, 0; p016,
CD.cDC2.CD1C.CD207, 0; p018, CD.cDC2.CD1C.CD207, 0; p019, CD.cDC2.CD1C.CD207, 0; p022, CD.cDC2.CD1C.CD207, 0; p024, CD.cDC2.CD1C.CD207, 0; p026,
CD.cDC2.CD1C.CD207, 0; p027, CD.cDC2.CD1C.CD207, 0; p031, CD.cDC2.CD1C.CD207, 0; p032, CD.cDC2.CD1C.CD207, 0; p041, CD.cDC2.CD1C.CD207, 0; p042,
CD.cDC2.CD1C.CD207, 0; p048, CD.cDC2.CD1C.CD207, 58; p011, CD.cDC2.CD1C.NDRG2, 0; p014, CD.cDC2.CD1C.NDRG2, 0; p016, CD.cDC2.CD1C.NDRG2, 0; p018,
CD.cDC2.CD1C.NDRG2, 18; p019, CD.cDC2.CD1C.NDRG2, 38; p022, CD.cDC2.CD1C.NDRG2, 1; p024, CD.cDC2.CD1C.NDRG2, 0; p026, CD.cDC2.CD1C.NDRG2, 1; p027,
CD.cDC2.CD1C.NDRG2, 24; p031, CD.cDC2.CD1C.NDRG2, 0; p032, CD.cDC2.CD1C.NDRG2, 0; p041, CD.cDC2.CD1C.NDRG2, 6; p042, CD.cDC2.CD1C.NDRG2, 73; p048,
CD.cDC2.CD1C.NDRG2, 0; p011, CD.cDC2.CLEC10A.CD1E, 1; p014, CD.cDC2.CLEC10A.CD1E, 0; p016, CD.cDC2.CLEC10A.CD1E, 1; p018, CD.cDC2.CLEC10A.CD1E, 11; p019,
CD.cDC2.CLEC10A.CD1E, 17; p022, CD.cDC2.CLEC10A.CD1E, 0; p024, CD.cDC2.CLEC10A.CD1E, 3; p026, CD.cDC2.CLEC10A.CD1E, 7; p027, CD.cDC2.CLEC10A.CD1E, 9; p031,
CD.cDC2.CLEC10A.CD1E, 0; p032, CD.cDC2.CLEC10A.CD1E, 5; p041, CD.cDC2.CLEC10A.CD1E, 3; p042, CD.cDC2.CLEC10A.CD1E, 8; p048, CD.cDC2.CLEC10A.CD1E, 0; p011,
CD.cDC2.CLEC10A.FCGR2B, 14; p014, CD.cDC2.CLEC10A.FCGR2B, 0; p016, CD.cDC2.CLEC10A.FCGR2B, 0; p018, CD.cDC2.CLEC10A.FCGR2B, 1; p019,
CD.cDC2.CLEC10A.FCGR2B, 2; p022, CD.cDC2.CLEC10A.FCGR2B, 1; p024, CD.cDC2.CLEC10A.FCGR2B, 0; p026, CD.cDC2.CLEC10A.FCGR2B, 1; p027,
CD.cDC2.CLEC10A.FCGR2B, 8; p031, CD.cDC2.CLEC10A.FCGR2B, 0; p032, CD.cDC2.CLEC10A.FCGR2B, 0; p041, CD.cDC2.CLEC10A.FCGR2B, 0; p042,
CD.cDC2.CLEC10A.FCGR2B, 8; p048, CD.cDC2.CLEC10A.FCGR2B, 39; p011, CD.cDC2.FCER1A.CD1C, 1; p014, CD.cDC2.FCER1A.CD1C, 0; p016, CD.cDC2.FCER1A.CD1C, 90;
p018, CD.cDC2.FCER1A.CD1C, 0; p019, CD.cDC2.FCER1A.CD1C, 0; p022, CD.cDC2.FCER1A.CD1C, 0; p024, CD.cDC2.FCER1A.CD1C, 0; p026, CD.cDC2.FCER1A.CD1C, 0; p027,
CD.cDC2.FCER1A.CD1C, 0; p031, CD.cDC2.FCER1A.CD1C, 0; p032, CD.cDC2.FCER1A.CD1C, 0; p041, CD.cDC2.FCER1A.CD1C, 0; p042, CD.cDC2.FCER1A.CD1C, 0; p048,
CD.cDC2.FCER1A.CD1C, 0; p011, CD.cDC2.MKI67.CD1C, 2; p014, CD.cDC2.MKI67.CD1C, 0; p016, CD.cDC2.MKI67.CD1C, 4; p018, CD.cDC2.MKI67.CD1C, 1; p019,
CD.cDC2.MKI67.CD1C, 0; p022, CD.cDC2.MKI67.CD1C, 0; p024, CD.cDC2.MKI67.CD1C, 1; p026, CD.cDC2.MKI67.CD1C, 1; p027, CD.cDC2.MKI67.CD1C, 1; p031,
CD.cDC2.MKI67.CD1C, 2; p032, CD.cDC2.MKI67.CD1C, 1; p041, CD.cDC2.MKI67.CD1C, 2; p042, CD.cDC2.MKI67.CD1C, 9; p048, CD.cDC2.MKI67.CD1C, 6; p011,
CD.cDC2.RALA.CD1C, 97; p014, CD.cDC2.RALA.CD1C, 0; p016, CD.cDC2.RALA.CD1C, 0; p018, CD.cDC2.RALA.CD1C, 0; p019, CD.cDC2.RALA.CD1C, 0; p022,
CD.cDC2.RALA.CD1C, 0; p024, CD.cDC2.RALA.CD1C, 0; p026, CD.cDC2.RALA.CD1C, 0; p027, CD.cDC2.RALA.CD1C, 0; p031, CD.cDC2.RALA.CD1C, 0; p032,
CD.cDC2.RALA.CD1C, 0; p041, CD.cDC2.RALA.CD1C, 0; p042, CD.cDC2.RALA.CD1C, 0; p048, CD.cDC2.RALA.CD1C, 0; p011, CD.cDC2.ZFAND2A.INHBA, 51; p014,
CD.cDC2.ZFAND2A.INHBA, 0; p016, CD.cDC2.ZFAND2A.INHBA, 0; p018, CD.cDC2.ZFAND2A.INHBA, 0; p019, CD.cDC2.ZFAND2A.INHBA, 0; p022, CD.cDC2.ZFAND2A.INHBA,
0; p024, CD.cDC2.ZFAND2A.INHBA, 0; p026, CD.cDC2.ZFAND2A.INHBA, 0; p027, CD.cDC2.ZFAND2A.INHBA, 0; p031, CD.cDC2.ZFAND2A.INHBA, 0; p032,
CD.cDC2.ZFAND2A.INHBA, 0; p041, CD.cDC2.ZFAND2A.INHBA, 0; p042, CD.cDC2.ZFAND2A.INHBA, 0; p048, CD.cDC2.ZFAND2A.INHBA, 0; p011, CD.DC.CCR7.FSCN1, 56;
p014, CD.DC.CCR7.FSCN1, 58; p016, CD.DC.CCR7.FSCN1, 21; p018, CD.DC.CCR7.FSCN1, 12; p019, CD.DC.CCR7.FSCN1, 34; p022, CD.DC.CCR7.FSCN1, 29; p024,
CD.DC.CCR7.FSCN1, 25; p026, CD.DC.CCR7.FSCN1, 12; p027, CD.DC.CCR7.FSCN1, 41; p031, CD.DC.CCR7.FSCN1, 56; p032, CD.DC.CCR7.FSCN1, 11; p041, CD.DC.CCR7.FSCN1,
56; p042, CD.DC.CCR7.FSCN1, 21; p048, CD.DC.CCR7.FSCN1, 48; p011, CD.DC.CXCL10.IDO1, 0; p014, CD.DC.CXCL10.IDO1, 9; p016, CD.DC.CXCL10.IDO1, 1; p018,
CD.DC.CXCL10.IDO1, 1; p019, CD.DC.CXCL10.IDO1, 1; p022, CD.DC.CXCL10.IDO1, 1; p024, CD.DC.CXCL10.IDO1, 3; p026, CD.DC.CXCL10.IDO1, 1; p027, CD.DC.CXCL10.IDO1,
6; p031, CD.DC.CXCL10.IDO1, 5; p032, CD.DC.CXCL10.IDO1, 7; p041, CD.DC.CXCL10.IDO1, 1; p042, CD.DC.CXCL10.IDO1, 3; p048, CD.DC.CXCL10.IDO1, 0; p011,
CD.DC.LTB.IL22RA2, 15; p014, CD.DC.LTB.IL22RA2, 1; p016, CD.DC.LTB.IL22RA2, 4; p018, CD.DC.LTB.IL22RA2, 8; p019, CD.DC.LTB.IL22RA2, 5; p022, CD.DC.LTB.IL22RA2,
2; p024, CD.DC.LTB.IL22RA2, 5; p026, CD.DC.LTB.IL22RA2, 1; p027, CD.DC.LTB.IL22RA2, 3; p031, CD.DC.LTB.IL22RA2, 9; p032, CD.DC.LTB.IL22RA2, 1; p041,
CD.DC.LTB.IL22RA2, 3; p042, CD.DC.LTB.IL22RA2, 12; p048, CD.DC.LTB.IL22RA2, 13; p011, CD.EC.ADH1C.EDN1, 0; p014, CD.EC.ADH1C.EDN1, 1; p016,
CD.EC.ADH1C.EDN1, 9; p018, CD.EC.ADH1C.EDN1, 16; p019, CD.EC.ADH1C.EDN1, 4; p022, CD.EC.ADH1C.EDN1, 0; p024, CD.EC.ADH1C.EDN1, 0; p026, CD.EC.ADH1C.EDN1,
0; p027, CD.EC.ADH1C.EDN1, 18; p031, CD.EC.ADH1C.EDN1, 0; p032, CD.EC.ADH1C.EDN1, 0; p041, CD.EC.ADH1C.EDN1, 1; p042, CD.EC.ADH1C.EDN1, 8; p048,
CD.EC.ADH1C.EDN1, 154; p011, CD.EC.ADH1C.GSTA1, 0; p014, CD.EC.ADH1C.GSTA1, 0; p016, CD.EC.ADH1C.GSTA1, 1; p018, CD.EC.ADH1C.GSTA1, 1; p019,
CD.EC.ADH1C.GSTA1, 5; p022, CD.EC.ADH1C.GSTA1, 0; p024, CD.EC.ADH1C.GSTA1, 0; p026, CD.EC.ADH1C.GSTA1, 0; p027, CD.EC.ADH1C.GSTA1, 52; p031,
CD.EC.ADH1C.GSTA1, 0; p032, CD.EC.ADH1C.GSTA1, 0; p041, CD.EC.ADH1C.GSTA1, 0; p042, CD.EC.ADH1C.GSTA1, 0; p048, CD.EC.ADH1C.GSTA1, 1; p011,
CD.EC.ADH1C.RPS4Y1, 0; p014, CD.EC.ADH1C.RPS4Y1, 0; p016, CD.EC.ADH1C.RPS4Y1, 17; p018, CD.EC.ADH1C.RPS4Y1, 1; p019, CD.EC.ADH1C.RPS4Y1, 8; p022,
CD.EC.ADH1C.RPS4Y1, 0; p024, CD.EC.ADH1C.RPS4Y1, 0; p026, CD.EC.ADH1C.RPS4Y1, 0; p027, CD.EC.ADH1C.RPS4Y1, 48; p031, CD.EC.ADH1C.RPS4Y1, 1; p032,
CD.EC.ADH1C.RPS4Y1, 0; p041, CD.EC.ADH1C.RPS4Y1, 0; p042, CD.EC.ADH1C.RPS4Y1, 5; p048, CD.EC.ADH1C.RPS4Y1, 3; p011, CD.EC.AKR1C1.TSPAN1, 0; p014,
CD.EC.AKR1C1.TSPAN1, 0; p016, CD.EC.AKR1C1.TSPAN1, 0; p018, CD.EC.AKR1C1.TSPAN1, 24; p019, CD.EC.AKR1C1.TSPAN1, 0; p022, CD.EC.AKR1C1.TSPAN1, 1; p024,
CD.EC.AKR1C1.TSPAN1, 0; p026, CD.EC.AKR1C1.TSPAN1, 0; p027, CD.EC.AKR1C1.TSPAN1, 1; p031, CD.EC.AKR1C1.TSPAN1, 0; p032, CD.EC.AKR1C1.TSPAN1, 0; p041,
CD.EC.AKR1C1.TSPAN1, 0; p042, CD.EC.AKR1C1.TSPAN1, 0; p048, CD.EC.AKR1C1.TSPAN1, 0; p011, CD.EC.ANPEP.DUOX2, 10; p014, CD.EC.ANPEP.DUOX2, 6; p016,
CD.EC.ANPEP.DUOX2, 3; p018, CD.EC.ANPEP.DUOX2, 29; p019, CD.EC.ANPEP.DUOX2, 1; p022, CD.EC.ANPEP.DUOX2, 16; p024, CD.EC.ANPEP.DUOX2, 7; p026,
CD.EC.ANPEP.DUOX2, 15; p027, CD.EC.ANPEP.DUOX2, 43; p031, CD.EC.ANPEP.DUOX2, 67; p032, CD.EC.ANPEP.DUOX2, 78; p041, CD.EC.ANPEP.DUOX2, 1; p042,
CD.EC.ANPEP.DUOX2, 1; p048, CD.EC.ANPEP.DUOX2, 28; p011, CD.EC.APOB.APOC3, 0; p014, CD.EC.APOB.APOC3, 173; p016, CD.EC.APOB.APOC3, 0; p018,
CD.EC.APOB.APOC3, 0; p019, CD.EC.APOB.APOC3, 1; p022, CD.EC.APOB.APOC3, 0; p024, CD.EC.APOB.APOC3, 0; p026, CD.EC.APOB.APOC3, 0; p027, CD.EC.APOB.APOC3, 3;
p031, CD.EC.APOB.APOC3, 0; p032, CD.EC.APOB.APOC3, 0; p041, CD.EC.APOB.APOC3, 0; p042, CD.EC.APOB.APOC3, 0; p048, CD.EC.APOB.APOC3, 14; p011,
CD.EC.APOC3.APOA4, 0; p014, CD.EC.APOC3.APOA4, 99; p016, CD.EC.APOC3.APOA4, 0; p018, CD.EC.APOC3.APOA4, 6; p019, CD.EC.APOC3.APOA4, 3; p022,
CD.EC.APOC3.APOA4, 0; p024, CD.EC.APOC3.APOA4, 0; p026, CD.EC.APOC3.APOA4, 0; p027, CD.EC.APOC3.APOA4, 0; p031, CD.EC.APOC3.APOA4, 0; p032,
CD.EC.APOC3.APOA4, 0; p041, CD.EC.APOC3.APOA4, 0; p042, CD.EC.APOC3.APOA4, 1; p048, CD.EC.APOC3.APOA4, 2; p011, CD.EC.APOC3.CUBN, 0; p014,
CD.EC.APOC3.CUBN, 424; p016, CD.EC.APOC3.CUBN, 0; p018, CD.EC.APOC3.CUBN, 0; p019, CD.EC.APOC3.CUBN, 2; p022, CD.EC.APOC3.CUBN, 0; p024, CD.EC.APOC3.CUBN,
0; p026, CD.EC.APOC3.CUBN, 0; p027, CD.EC.APOC3.CUBN, 2; p031, CD.EC.APOC3.CUBN, 2; p032, CD.EC.APOC3.CUBN, 0; p041, CD.EC.APOC3.CUBN, 0; p042,
CD.EC.APOC3.CUBN, 0; p048, CD.EC.APOC3.CUBN, 0; p011, CD.EC.ARHGDIG.SULT1E1, 0; p014, CD.EC.ARHGDIG.SULT1E1, 0; p016, CD.EC.ARHGDIG.SULT1E1, 0; p018,
CD.EC.ARHGDIG.SULT1E1, 0; p019, CD.EC.ARHGDIG.SULT1E1, 0; p022, CD.EC.ARHGDIG.SULT1E1, 0; p024, CD.EC.ARHGDIG.SULT1E1, 0; p026, CD.EC.ARHGDIG.SULT1E1,
7; p027, CD.EC.ARHGDIG.SULT1E1, 0; p031, CD.EC.ARHGDIG.SULT1E1, 5; p032, CD.EC.ARHGDIG.SULT1E1, 1; p041, CD.EC.ARHGDIG.SULT1E1, 0; p042,
CD.EC.ARHGDIG.SULT1E1, 0; p048, CD.EC.ARHGDIG.SULT1E1, 0; p011, CD.EC.CA1.TMPRSS15, 1; p014, CD.EC.CA1.TMPRSS15, 1; p016, CD.EC.CA1.TMPRSS15, 0; p018,
CD.EC.CA1.TMPRSS15, 0; p019, CD.EC.CA1.TMPRSS15, 0; p022, CD.EC.CA1.TMPRSS15, 4; p024, CD.EC.CA1.TMPRSS15, 0; p026, CD.EC.CA1.TMPRSS15, 0; p027,
CD.EC.CA1.TMPRSS15, 1; p031, CD.EC.CA1.TMPRSS15, 0; p032, CD.EC.CA1.TMPRSS15, 5; p041, CD.EC.CA1.TMPRSS15, 1; p042, CD.EC.CA1.TMPRSS15, 0; p048,
CD.EC.CA1.TMPRSS15, 0; p011, CD.EC.CFTR.OLFM4, 0; p014, CD.EC.CFTR.OLFM4, 178; p016, CD.EC.CFTR.OLFM4, 0; p018, CD.EC.CFTR.OLFM4, 0; p019,
CD.EC.CFTR.OLFM4, 0; p022, CD.EC.CFTR.OLFM4, 0; p024, CD.EC.CFTR.OLFM4, 0; p026, CD.EC.CFTR.OLFM4, 0; p027, CD.EC.CFTR.OLFM4, 0; p031, CD.EC.CFTR.OLFM4, 1;
p032, CD.EC.CFTR.OLFM4, 1; p041, CD.EC.CFTR.OLFM4, 0; p042, CD.EC.CFTR.OLFM4, 0; p048, CD.EC.CFTR.OLFM4, 0; p011, CD.EC.FABP1.ADIRF, 0; p014,
CD.EC.FABP1.ADIRF, 7; p016, CD.EC.FABP1.ADIRF, 2; p018, CD.EC.FABP1.ADIRF, 78; p019, CD.EC.FABP1.ADIRF, 24; p022, CD.EC.FABP1.ADIRF, 8; p024,
CD.EC.FABP1.ADIRF, 0; p026, CD.EC.FABP1.ADIRF, 25; p027, CD.EC.FABP1.ADIRF, 47; p031, CD.EC.FABP1.ADIRF, 36; p032, CD.EC.FABP1.ADIRF, 17; p041,
CD.EC.FABP1.ADIRF, 1; p042, CD.EC.FABP1.ADIRF, 1; p048, CD.EC.FABP1.ADIRF, 54; p011, CD.EC.FABP6.PLCG2, 0; p014, CD.EC.FABP6.PLCG2, 6; p016,
CD.EC.FABP6.PLCG2, 0; p018, CD.EC.FABP6.PLCG2, 1; p019, CD.EC.FABP6.PLCG2, 1; p022, CD.EC.FABP6.PLCG2, 1; p024, CD.EC.FABP6.PLCG2, 0; p026,
CD.EC.FABP6.PLCG2, 6; p027, CD.EC.FABP6.PLCG2, 32; p031, CD.EC.FABP6.PLCG2, 15; p032, CD.EC.FABP6.PLCG2, 4; p041, CD.EC.FABP6.PLCG2, 0; p042,
CD.EC.FABP6.PLCG2, 0; p048, CD.EC.FABP6.PLCG2, 71; p011, CD.EC.FABP6.SLC26A3, 0; p014, CD.EC.FABP6.SLC26A3, 247; p016, CD.EC.FABP6.SLC26A3, 0; p018,
CD.EC.FABP6.SLC26A3, 0; p019, CD.EC.FABP6.SLC26A3, 0; p022, CD.EC.FABP6.SLC26A3, 0; p024, CD.EC.FABP6.SLC26A3, 0; p026, CD.EC.FABP6.SLC26A3, 0; p027,
CD.EC.FABP6.SLC26A3, 0; p031, CD.EC.FABP6.SLC26A3, 1; p032, CD.EC.FABP6.SLC26A3, 0; p041, CD.EC.FABP6.SLC26A3, 0; p042, CD.EC.FABP6.SLC26A3, 0; p048,
CD.EC.FABP6.SLC26A3, 1; p011, CD.EC.GNAT3.TRPM5, 0; p014, CD.EC.GNAT3.TRPM5, 11; p016, CD.EC.GNAT3.TRPM5, 1; p018, CD.EC.GNAT3.TRPM5, 1; p019,
CD.EC.GNAT3.TRPM5, 1; p022, CD.EC.GNAT3.TRPM5, 0; p024, CD.EC.GNAT3.TRPM5, 0; p026, CD.EC.GNAT3.TRPM5, 1; p027, CD.EC.GNAT3.TRPM5, 12; p031,
CD.EC.GNAT3.TRPM5, 2; p032, CD.EC.GNAT3.TRPM5, 0; p041, CD.EC.GNAT3.TRPM5, 0; p042, CD.EC.GNAT3.TRPM5, 0; p048, CD.EC.GNAT3.TRPM5, 26; p011,
CD.EC.GSTA2.AADAC, 0; p014, CD.EC.GSTA2.AADAC, 0; p016, CD.EC.GSTA2.AADAC, 0; p018, CD.EC.GSTA2.AADAC, 0; p019, CD.EC.GSTA2.AADAC, 0; p022,
CD.EC.GSTA2.AADAC, 0; p024, CD.EC.GSTA2.AADAC, 0; p026, CD.EC.GSTA2.AADAC, 2; p027, CD.EC.GSTA2.AADAC, 17; p031, CD.EC.GSTA2.AADAC, 0; p032,
CD.EC.GSTA2.AADAC, 2; p041, CD.EC.GSTA2.AADAC, 0; p042, CD.EC.GSTA2.AADAC, 0; p048, CD.EC.GSTA2.AADAC, 1; p011, CD.EC.GSTA2.CES3, 0; p014, CD.EC.GSTA2.CES3,
0; p016, CD.EC.GSTA2.CES3, 10; p018, CD.EC.GSTA2.CES3, 0; p019, CD.EC.GSTA2.CES3, 1; p022, CD.EC.GSTA2.CES3, 1; p024, CD.EC.GSTA2.CES3, 0; p026,
CD.EC.GSTA2.CES3, 0; p027, CD.EC.GSTA2.CES3, 2; p031, CD.EC.GSTA2.CES3, 0; p032, CD.EC.GSTA2.CES3, 0; p041, CD.EC.GSTA2.CES3, 0; p042, CD.EC.GSTA2.CES3, 0; p048,
CD.EC.GSTA2.CES3, 6; p011, CD.EC.GSTA2.TMPRSS15, 0; p014, CD.EC.GSTA2.TMPRSS15, 0; p016, CD.EC.GSTA2.TMPRSS15, 0; p018, CD.EC.GSTA2.TMPRSS15, 0; p019,
CD.EC.GSTA2.TMPRSS15, 2; p022, CD.EC.GSTA2.TMPRSS15, 0; p024, CD.EC.GSTA2.TMPRSS15, 0; p026, CD.EC.GSTA2.TMPRSS15, 0; p027, CD.EC.GSTA2.TMPRSS15, 20;
p031, CD.EC.GSTA2.TMPRSS15, 0; p032, CD.EC.GSTA2.TMPRSS15, 0; p041, CD.EC.GSTA2.TMPRSS15, 0; p042, CD.EC.GSTA2.TMPRSS15, 1; p048, CD.EC.GSTA2.TMPRSS15,
2; p011, CD.EC.HSPA1B.PSMA2, 0; p014, CD.EC.HSPA1B.PSMA2, 0; p016, CD.EC.HSPA1B.PSMA2, 0; p018, CD.EC.HSPA1B.PSMA2, 0; p019, CD.EC.HSPA1B.PSMA2, 0; p022,
CD.EC.HSPA1B.PSMA2, 0; p024, CD.EC.HSPA1B.PSMA2, 0; p026, CD.EC.HSPA1B.PSMA2, 0; p027, CD.EC.HSPA1B.PSMA2, 0; p031, CD.EC.HSPA1B.PSMA2, 65; p032,
CD.EC.HSPA1B.PSMA2, 0; p041, CD.EC.HSPA1B.PSMA2, 0; p042, CD.EC.HSPA1B.PSMA2, 0; p048, CD.EC.HSPA1B.PSMA2, 0; p011, CD.EC.IFI6.IFI27, 2; p014,
CD.EC.IFI6.IFI27, 0; p016, CD.EC.IFI6.IFI27, 1; p018, CD.EC.IFI6.IFI27, 6; p019, CD.EC.IFI6.IFI27, 2; p022, CD.EC.IFI6.IFI27, 5; p024, CD.EC.IFI6.IFI27, 0; p026,
CD.EC.IFI6.IFI27, 115; p027, CD.EC.IFI6.IFI27, 9; p031, CD.EC.IFI6.IFI27, 1; p032, CD.EC.IFI6.IFI27, 1; p041, CD.EC.IFI6.IFI27, 0; p042, CD.EC.IFI6.IFI27, 0; p048,
CD.EC.IFI6.IFI27, 0; p011, CD.EC.LECT1.FAM26F, 0; p014, CD.EC.LECT1.FAM26F, 0; p016, CD.EC.LECT1.FAM26F, 0; p018, CD.EC.LECT1.FAM26F, 1; p019,
CD.EC.LECT1.FAM26F, 4; p022, CD.EC.LECT1.FAM26F, 0; p024, CD.EC.LECT1.FAM26F, 0; p026, CD.EC.LECT1.FAM26F, 1; p027, CD.EC.LECT1.FAM26F, 67; p031,
CD.EC.LECT1.FAM26F, 1; p032, CD.EC.LECT1.FAM26F, 1; p041, CD.EC.LECT1.FAM26F, 0; p042, CD.EC.LECT1.FAM26F, 6; p048, CD.EC.LECT1.FAM26F, 0; p011,
CD.EC.LYZ.OLFM4, 1; p014, CD.EC.LYZ.OLFM4, 1; p016, CD.EC.LYZ.OLFM4, 0; p018, CD.EC.LYZ.OLFM4, 2; p019, CD.EC.LYZ.OLFM4, 0; p022, CD.EC.LYZ.OLFM4, 38; p024,
CD.EC.LYZ.OLFM4, 2; p026, CD.EC.LYZ.OLFM4, 3; p027, CD.EC.LYZ.OLFM4, 0; p031, CD.EC.LYZ.OLFM4, 2; p032, CD.EC.LYZ.OLFM4, 3; p041, CD.EC.LYZ.OLFM4, 5; p042,
CD.EC.LYZ.OLFM4, 0; p048, CD.EC.LYZ.OLFM4, 2; p011, CD.EC.MIF.AC0904981, 66; p014, CD.EC.MIF.AC0904981, 58; p016, CD.EC.MIF.AC0904981, 8; p018,
CD.EC.MIF.AC0904981, 6; p019, CD.EC.MIF.AC0904981, 2; p022, CD.EC.MIF.AC0904981, 6; p024, CD.EC.MIF.AC0904981, 7; p026, CD.EC.MIF.AC0904981, 2; p027,
CD.EC.MIF.AC0904981, 24; p031, CD.EC.MIF.AC0904981, 9; p032, CD.EC.MIF.AC0904981, 21; p041, CD.EC.MIF.AC0904981, 8; p042, CD.EC.MIF.AC0904981, 1; p048,
CD.EC.MIF.AC0904981, 4; p011, CD.EC.MIF.MTRNR2L8, 4; p014, CD.EC.MIF.MTRNR2L8, 224; p016, CD.EC.MIF.MTRNR2L8, 0; p018, CD.EC.MIF.MTRNR2L8, 0; p019,
CD.EC.MIF.MTRNR2L8, 0; p022, CD.EC.MIF.MTRNR2L8, 0; p024, CD.EC.MIF.MTRNR2L8, 1; p026, CD.EC.MIF.MTRNR2L8, 0; p027, CD.EC.MIF.MTRNR2L8, 0; p031,
CD.EC.MIF.MTRNR2L8, 0; p032, CD.EC.MIF.MTRNR2L8, 1; p041, CD.EC.MIF.MTRNR2L8, 0; p042, CD.EC.MIF.MTRNR2L8, 0; p048, CD.EC.MIF.MTRNR2L8, 3; p011,
CD.EC.MT1H.MT1G, 1; p014, CD.EC.MT1H.MT1G, 1; p016, CD.EC.MT1H.MT1G, 4; p018, CD.EC.MT1H.MT1G, 59; p019, CD.EC.MT1H.MT1G, 14; p022, CD.EC.MT1H.MT1G,
109; p024, CD.EC.MT1H.MT1G, 13; p026, CD.EC.MT1H.MT1G, 0; p027, CD.EC.MT1H.MT1G, 6; p031, CD.EC.MT1H.MT1G, 0; p032, CD.EC.MT1H.MT1G, 0; p041,
CD.EC.MT1H.MT1G, 0; p042, CD.EC.MT1H.MT1G, 1; p048, CD.EC.MT1H.MT1G, 36; p011, CD.EC.MT1H.PHGDH, 0; p014, CD.EC.MT1H.PHGDH, 0; p016, CD.EC.MT1H.PHGDH,
0; p018, CD.EC.MT1H.PHGDH, 107; p019, CD.EC.MT1H.PHGDH, 0; p022, CD.EC.MT1H.PHGDH, 0; p024, CD.EC.MT1H.PHGDH, 0; p026, CD.EC.MT1H.PHGDH, 0; p027,
CD.EC.MT1H.PHGDH, 0; p031, CD.EC.MT1H.PHGDH, 0; p032, CD.EC.MT1H.PHGDH, 0; p041, CD.EC.MT1H.PHGDH, 0; p042, CD.EC.MT1H.PHGDH, 0; p048,
CD.EC.MT1H.PHGDH, 0; p011, CD.EC.MTRNR2L1.MT-ND3, 145; p014, CD.EC.MTRNR2L1.MT-ND3, 98; p016, CD.EC.MTRNR2L1.MT-ND3, 68; p018, CD.EC.MTRNR2L1.MT-
ND3, 124; p019, CD.EC.MTRNR2L1.MT-ND3, 17; p022, CD.EC.MTRNR2L1.MT-ND3, 60; p024, CD.EC.MTRNR2L1.MT-ND3, 9; p026, CD.EC.MTRNR2L1.MT-ND3, 18; p027,
CD.EC.MTRNR2L1.MT-ND3, 109; p031, CD.EC.MTRNR2L1.MT-ND3, 176; p032, CD.EC.MTRNR2L1.MT-ND3, 49; p041, CD.EC.MTRNR2L1.MT-ND3, 15; p042,
CD.EC.MTRNR2L1.MT-ND3, 12; p048, CD.EC.MTRNR2L1.MT-ND3, 709; p011, CD.EC.NUPR1.LCN2, 4; p014, CD.EC.NUPR1.LCN2, 0; p016, CD.EC.NUPR1.LCN2, 0; p018,
CD.EC.NUPR1.LCN2, 0; p019, CD.EC.NUPR1.LCN2, 0; p022, CD.EC.NUPR1.LCN2, 2; p024, CD.EC.NUPR1.LCN2, 36; p026, CD.EC.NUPR1.LCN2, 0; p027, CD.EC.NUPR1.LCN2,
0; p031, CD.EC.NUPR1.LCN2, 51; p032, CD.EC.NUPR1.LCN2, 0; p041, CD.EC.NUPR1.LCN2, 1; p042, CD.EC.NUPR1.LCN2, 0; p048, CD.EC.NUPR1.LCN2, 0; p011,
CD.EC.OLFM4.MT-ND2, 9; p014, CD.EC.OLFM4.MT-ND2, 5; p016, CD.EC.OLFM4.MT-ND2, 7; p018, CD.EC.OLFM4.MT-ND2, 28; p019, CD.EC.OLFM4.MT-ND2, 0; p022,
CD.EC.OLFM4.MT-ND2, 33; p024, CD.EC.OLFM4.MT-ND2, 41; p026, CD.EC.OLFM4.MT-ND2, 13; p027, CD.EC.OLFM4.MT-ND2, 1; p031, CD.EC.OLFM4.MT-ND2, 37; p032,
CD.EC.OLFM4.MT-ND2, 1; p041, CD.EC.OLFM4.MT-ND2, 24; p042, CD.EC.OLFM4.MT-ND2, 3; p048, CD.EC.OLFM4.MT-ND2, 31; p011, CD.EC.PLCG2.MAFB, 55; p014,
CD.EC.PLCG2.MAFB, 9; p016, CD.EC.PLCG2.MAFB, 5; p018, CD.EC.PLCG2.MAFB, 13; p019, CD.EC.PLCG2.MAFB, 0; p022, CD.EC.PLCG2.MAFB, 5; p024, CD.EC.PLCG2.MAFB,
14; p026, CD.EC.PLCG2.MAFB, 15; p027, CD.EC.PLCG2.MAFB, 19; p031, CD.EC.PLCG2.MAFB, 45; p032, CD.EC.PLCG2.MAFB, 22; p041, CD.EC.PLCG2.MAFB, 4; p042,
CD.EC.PLCG2.MAFB, 1; p048, CD.EC.PLCG2.MAFB, 159; p011, CD.EC.PLCG2.MTRNR2L8, 1; p014, CD.EC.PLCG2.MTRNR2L8, 264; p016, CD.EC.PLCG2.MTRNR2L8, 1; p018,
CD.EC.PLCG2.MTRNR2L8, 0; p019, CD.EC.PLCG2.MTRNR2L8, 0; p022, CD.EC.PLCG2.MTRNR2L8, 0; p024, CD.EC.PLCG2.MTRNR2L8, 0; p026, CD.EC.PLCG2.MTRNR2L8, 0;
p027, CD.EC.PLCG2.MTRNR2L8, 0; p031, CD.EC.PLCG2.MTRNR2L8, 0; p032, CD.EC.PLCG2.MTRNR2L8, 0; p041, CD.EC.PLCG2.MTRNR2L8, 0; p042,
CD.EC.PLCG2.MTRNR2L8, 0; p048, CD.EC.PLCG2.MTRNR2L8, 3; p011, CD.EC.PLCG2.RP11-727F159, 33; p014, CD.EC.PLCG2.RP11-727F159, 4; p016, CD.EC.PLCG2.RP11-
727F159, 7; p018, CD.EC.PLCG2.RP11-727F159, 43; p019, CD.EC.PLCG2.RP11-727F159, 0; p022, CD.EC.PLCG2.RP11-727F159, 2; p024, CD.EC.PLCG2.RP11-727F159, 13;
p026, CD.EC.PLCG2.RP11-727F159, 34; p027, CD.EC.PLCG2.RP11-727F159, 28; p031, CD.EC.PLCG2.RP11-727F159, 75; p032, CD.EC.PLCG2.RP11-727F159, 9; p041,
CD.EC.PLCG2.RP11-727F159, 3; p042, CD.EC.PLCG2.RP11-727F159, 2; p048, CD.EC.PLCG2.RP11-727F159, 223; p011, CD.EC.RBP2.ALPI, 0; p014, CD.EC.RBP2.ALPI, 1;
p016, CD.EC.RBP2.ALPI, 0; p018, CD.EC.RBP2.ALPI, 160; p019, CD.EC.RBP2.ALPI, 3; p022, CD.EC.RBP2.ALPI, 0; p024, CD.EC.RBP2.ALPI, 0; p026, CD.EC.RBP2.ALPI, 2; p027,
CD.EC.RBP2.ALPI, 2; p031, CD.EC.RBP2.ALPI, 1; p032, CD.EC.RBP2.ALPI, 8; p041, CD.EC.RBP2.ALPI, 0; p042, CD.EC.RBP2.ALPI, 0; p048, CD.EC.RBP2.ALPI, 3; p011,
CD.EC.RBP2.CYP3A4, 0; p014, CD.EC.RBP2.CYP3A4, 11; p016, CD.EC.RBP2.CYP3A4, 0; p018, CD.EC.RBP2.CYP3A4, 0; p019, CD.EC.RBP2.CYP3A4, 19; p022,
CD.EC.RBP2.CYP3A4, 0; p024, CD.EC.RBP2.CYP3A4, 0; p026, CD.EC.RBP2.CYP3A4, 2; p027, CD.EC.RBP2.CYP3A4, 60; p031, CD.EC.RBP2.CYP3A4, 4; p032,
CD.EC.RBP2.CYP3A4, 8; p041, CD.EC.RBP2.CYP3A4, 0; p042, CD.EC.RBP2.CYP3A4, 3; p048, CD.EC.RBP2.CYP3A4, 16; p011, CD.EC.SLC28A2.GSTA2, 0; p014,
CD.EC.SLC28A2.GSTA2, 0; p016, CD.EC.SLC28A2.GSTA2, 0; p018, CD.EC.SLC28A2.GSTA2, 0; p019, CD.EC.SLC28A2.GSTA2, 20; p022, CD.EC.SLC28A2.GSTA2, 0; p024,
CD.EC.SLC28A2.GSTA2, 0; p026, CD.EC.SLC28A2.GSTA2, 1; p027, CD.EC.SLC28A2.GSTA2, 4; p031, CD.EC.SLC28A2.GSTA2, 0; p032, CD.EC.SLC28A2.GSTA2, 0; p041,
CD.EC.SLC28A2.GSTA2, 0; p042, CD.EC.SLC28A2.GSTA2, 0; p048, CD.EC.SLC28A2.GSTA2, 0; p011, CD.EC.SMOC2.ASCL2, 2; p014, CD.EC.SMOC2.ASCL2, 1; p016,
CD.EC.SMOC2.ASCL2, 0; p018, CD.EC.SMOC2.ASCL2, 0; p019, CD.EC.SMOC2.ASCL2, 0; p022, CD.EC.SMOC2.ASCL2, 0; p024, CD.EC.SMOC2.ASCL2, 3; p026,
CD.EC.SMOC2.ASCL2, 1; p027, CD.EC.SMOC2.ASCL2, 5; p031, CD.EC.SMOC2.ASCL2, 179; p032, CD.EC.SMOC2.ASCL2, 0; p041, CD.EC.SMOC2.ASCL2, 0; p042,
CD.EC.SMOC2.ASCL2, 0; p048, CD.EC.SMOC2.ASCL2, 1; p011, CD.EC.TCL1A.IFI6, 3; p014, CD.EC.TCL1A.IFI6, 0; p016, CD.EC.TCL1A.IFI6, 0; p018, CD.EC.TCL1A.IF16, 0; p019,
CD.EC.TCL1A.IFI6, 3; p022, CD.EC.TCL1A.IFI6, 1; p024, CD.EC.TCL1A.IFI6, 1; p026, CD.EC.TCL1A.IFI6, 315; p027, CD.EC.TCL1A.IFI6, 0; p031, CD.EC.TCL1A.IF16, 8; p032,
CD.EC.TCL1A.IFI6, 1; p041, CD.EC.TCL1A.IFI6, 0; p042, CD.EC.TCL1A.IFI6, 0; p048, CD.EC.TCL1A.IFI6, 1; p011, CD.EC.TMPRSS15.FAM8A1, 0; p014,
CD.EC.TMPRSS15.FAM8A1, 3; p016, CD.EC.TMPRSS15.FAM8A1, 0; p018, CD.EC.TMPRSS15.FAM8A1, 0; p019, CD.EC.TMPRSS15.FAM8A1, 0; p022,
CD.EC.TMPRSS15.FAM8A1, 1; p024, CD.EC.TMPRSS15.FAM8A1, 0; p026, CD.EC.TMPRSS15.FAM8A1, 0; p027, CD.EC.TMPRSS15.FAM8A1, 0; p031,
CD.EC.TMPRSS15.FAM8A1, 29; p032, CD.EC.TMPRSS15.FAM8A1, 1; p041, CD.EC.TMPRSS15.FAM8A1, 0; p042, CD.EC.TMPRSS15.FAM8A1, 0; p048,
CD.EC.TMPRSS15.FAM8A1, 0; p011, CD.EC.UBD.IFITM1, 0; p014, CD.EC.UBD.IFITM1, 0; p016, CD.EC.UBD.IFITM1, 0; p018, CD.EC.UBD.IFITM1, 0; p019, CD.EC.UBD.IFITM1,
0; p022, CD.EC.UBD.IFITM1, 1; p024, CD.EC.UBD.IFITM1, 0; p026, CD.EC.UBD.IFITM1, 1; p027, CD.EC.UBD.IFITM1, 0; p031, CD.EC.UBD.IFITM1, 0; p032, CD.EC.UBD.IFITM1,
47; p041, CD.EC.UBD.IFITM1, 0; p042, CD.EC.UBD.IFITM1, 0; p048, CD.EC.UBD.IFITM1, 0; p011, CD.Endth.IFI6.ISG15, 1; p014, CD.Endth.IFI6.ISG15, 0; p016,
CD.Endth.IFI6.ISG15, 0; p018, CD.Endth.IFI6.ISG15, 0; p019, CD.Endth.IFI6.ISG15, 0; p022, CD.Endth.IFI6.ISG15, 0; p024, CD.Endth.IFI6.ISG15, 0; p026,
CD.Endth.IFI6.ISG15, 308; p027, CD.Endth.IFI6.ISG15, 1; p031, CD.Endth.IFI6.ISG15, 1; p032, CD.Endth.IFI6.ISG15, 0; p041, CD.Endth.IFI6.ISG15, 0; p042,
CD.Endth.IFI6.ISG15, 0; p048, CD.Endth.IFI6.ISG15, 1; p011, CD.Endth.MT-CO3.MT-CO2, 23; p014, CD.Endth.MT-CO3.MT-CO2, 16; p016, CD.Endth.MT-CO3.MT-CO2, 26;
p018, CD.Endth.MT-CO3.MT-CO2, 24; p019, CD.Endth.MT-CO3.MT-CO2, 5; p022, CD.Endth.MT-CO3.MT-CO2, 56; p024, CD.Endth.MT-CO3.MT-CO2, 8; p026, CD.Endth.MT-
CO3.MT-CO2, 3; p027, CD.Endth.MT-CO3.MT-CO2, 29; p031, CD.Endth.MT-CO3.MT-CO2, 20; p032, CD.Endth.MT-CO3.MT-CO2, 11; p041, CD.Endth.MT-CO3.MT-CO2, 24;
p042, CD.Endth.MT-CO3.MT-CO2, 5; p048, CD.Endth.MT-CO3.MT-CO2, 37; p011, CD.Endth/Art.SEMA3G.SSUH2, 19; p014, CD.Endth/Art.SEMA3G.SSUH2, 4; p016,
CD.Endth/Art.SEMA3G.SSUH2, 4; p018, CD.Endth/Art.SEMA3G.SSUH2, 8; p019, CD.Endth/Art.SEMA3G.SSUH2, 10; p022, CD.Endth/Art.SEMA3G.SSUH2, 21; p024,
CD.Endth/Art.SEMA3G.SSUH2, 12; p026, CD.Endth/Art.SEMA3G.SSUH2, 3; p027, CD.Endth/Art.SEMA3G.SSUH2, 13; p031, CD.Endth/Art.SEMA3G.SSUH2, 3; p032,
CD.Endth/Art.SEMA3G.SSUH2, 2; p041, CD.Endth/Art.SEMA3G.SSUH2, 13; p042, CD.Endth/Art.SEMA3G.SSUH2, 11; p048, CD.Endth/Art.SEMA3G.SSUH2, 3; p011,
CD.Endth/Cap.CD36.FLT1, 118; p014, CD.Endth/Cap.CD36.FLT1, 20; p016, CD.Endth/Cap.CD36.FLT1, 40; p018, CD.Endth/Cap.CD36.FLT1, 59; p019,
CD.Endth/Cap.CD36.FLT1, 8; p022, CD.Endth/Cap.CD36.FLT1, 37; p024, CD.Endth/Cap.CD36.FLT1, 53; p026, CD.Endth/Cap.CD36.FLT1, 2; p027,
CD.Endth/Cap.CD36.FLT1, 24; p031, CD.Endth/Cap.CD36.FLT1, 51; p032, CD.Endth/Cap.CD36.FLT1, 16; p041, CD.Endth/Cap.CD36.FLT1, 26; p042,
CD.Endth/Cap.CD36.FLT1, 23; p048, CD.Endth/Cap.CD36.FLT1, 66; p011, CD.Endth/Lymph.CCL21.DKK3, 0; p014, CD.Endth/Lymph.CCL21.DKK3, 0; p016,
CD.Endth/Lymph.CCL21.DKK3, 30; p018, CD.Endth/Lymph.CCL21.DKK3, 0; p019, CD.Endth/Lymph.CCL21.DKK3, 0; p022, CD.Endth/Lymph.CCL21.DKK3, 0; p024,
CD.Endth/Lymph.CCL21.DKK3, 0; p026, CD.Endth/Lymph.CCL21.DKK3, 0; p027, CD.Endth/Lymph.CCL21.DKK3, 0; p031, CD.Endth/Lymph.CCL21.DKK3, 0; p032,
CD.Endth/Lymph.CCL21.DKK3, 0; p041, CD.Endth/Lymph.CCL21.DKK3, 0; p042, CD.Endth/Lymph.CCL21.DKK3, 0; p048, CD.Endth/Lymph.CCL21.DKK3, 0; p011,
CD.Endth/Lymph.CCL21.LYVE1, 0; p014, CD.Endth/Lymph.CCL21.LYVE1, 2; p016, CD.Endth/Lymph.CCL21.LYVE1, 0; p018, CD.Endth/Lymph.CCL21.LYVE1, 0; p019,
CD.Endth/Lymph.CCL21.LYVE1, 1; p022, CD.Endth/Lymph.CCL21.LYVE1, 6; p024, CD.Endth/Lymph.CCL21.LYVE1, 71; p026, CD.Endth/Lymph.CCL21.LYVE1, 0; p027,
CD.Endth/Lymph.CCL21.LYVE1, 0; p031, CD.Endth/Lymph.CCL21.LYVE1, 0; p032, CD.Endth/Lymph.CCL21.LYVE1, 1; p041, CD.Endth/Lymph.CCL21.LYVE1, 0; p042,
CD.Endth/Lymph.CCL21.LYVE1, 0; p048, CD.Endth/Lymph.CCL21.LYVE1, 0; p011, CD.Endth/Lymph.CCL21.PROX1, 0; p014, CD.Endth/Lymph.CCL21.PROX1, 1; p016,
CD.Endth/Lymph.CCL21.PROX1, 1; p018, CD.Endth/Lymph.CCL21.PROX1, 14; p019, CD.Endth/Lymph.CCL21.PROX1, 3; p022, CD.Endth/Lymph.CCL21.PROX1, 8; p024,
CD.Endth/Lymph.CCL21.PROX1, 10; p026, CD.Endth/Lymph.CCL21.PROX1, 0; p027, CD.Endth/Lymph.CCL21.PROX1, 1; p031, CD.Endth/Lymph.CCL21.PROX1, 15; p032,
CD.Endth/Lymph.CCL21.PROX1, 1; p041, CD.Endth/Lymph.CCL21.PROX1, 9; p042, CD.Endth/Lymph.CCL21.PROX1, 4; p048, CD.Endth/Lymph.CCL21.PROX1, 0; p011,
CD.Endth/Lymph.EFEMP1.CCL21, 0; p014, CD.Endth/Lymph.EFEMP1.CCL21, 0; p016, CD.Endth/Lymph.EFEMP1.CCL21, 0; p018, CD.Endth/Lymph.EFEMP1.CCL21, 7;
p019, CD.Endth/Lymph.EFEMP1.CCL21, 9; p022, CD.Endth/Lymph.EFEMP1.CCL21, 6; p024, CD.Endth/Lymph.EFEMP1.CCL21, 0; p026, CD.Endth/Lymph.EFEMP1.CCL21,
2; p027, CD.Endth/Lymph.EFEMP1.CCL21, 0; p031, CD.Endth/Lymph.EFEMP1.CCL21, 1; p032, CD.Endth/Lymph.EFEMP1.CCL21, 8; p041,
CD.Endth/Lymph.EFEMP1.CCL21, 25; p042, CD.Endth/Lymph.EFEMP1.CCL21, 0; p048, CD.Endth/Lymph.EFEMP1.CCL21, 0; p011, CD.Endth/Lymph.EFEMP1.LYVE1, 0;
p014, CD.Endth/Lymph.EFEMP1.LYVE1, 0; p016, CD.Endth/Lymph.EFEMP1.LYVE1, 0; p018, CD.Endth/Lymph.EFEMP1.LYVE1, 0; p019,
CD.Endth/Lymph.EFEMP1.LYVE1, 0; p022, CD.Endth/Lymph.EFEMP1.LYVE1, 0; p024, CD.Endth/Lymph.EFEMP1.LYVE1, 0; p026, CD.Endth/Lymph.EFEMP1.LYVE1, 0;
p027, CD.Endth/Lymph.EFEMP1.LYVE1, 0; p031, CD, Endth/Lymph.EFEMP1.LYVE1, 40; p032, CD.Endth/Lymph.EFEMP1.LYVE1, 0; p041,
CD.Endth/Lymph.EFEMP1.LYVE1, 0; p042, CD.Endth/Lymph.EFEMP1.LYVE1, 0; p048, CD.Endth/Lymph.EFEMP1.LYVE1, 0; p011, CD.Endth/Lymph.LY6H.CCL21, 0; p014,
CD.Endth/Lymph.LY6H.CCL21, 0; p016, CD.Endth/Lymph.LY6H.CCL21, 0; p018, CD.Endth/Lymph.LY6H.CCL21, 3; p019, CD.Endth/Lymph.LY6H.CCL21, 2; p022,
CD.Endth/Lymph.LY6H.CCL21, 0; p024, CD.Endth/Lymph.LY6H.CCL21, 0; p026, CD.Endth/Lymph.LY6H.CCL21, 1; p027, CD.Endth/Lymph.LY6H.CCL21, 3; p031,
CD.Endth/Lymph.LY6H.CCL21, 0; p032, CD.Endth/Lymph.LY6H.CCL21, 0; p041, CD.Endth/Lymph.LY6H.CCL21, 6; p042, CD.Endth/Lymph.LY6H.CCL21, 55; p048,
CD.Endth/Lymph.LY6H.CCL21, 1; p011, CD.Endth/Mural.HIGD1B.NDUFA4L2, 24; p014, CD.Endth/Mural.HIGD1B.NDUFA4L2, 5; p016,
CD.Endth/Mural.HIGD1B.NDUFA4L2, 7; p018, CD.Endth/Mural.HIGD1B.NDUFA4L2, 31; p019, CD.Endth/Mural.HIGD1B.NDUFA4L2, 2; p022,
CD.Endth/Mural.HIGD1B.NDUFA4L2, 3; p024, CD.Endth/Mural.HIGD1B.NDUFA4L2, 5; p026, CD.Endth/Mural.HIGD1B.NDUFA4L2, 16; p027,
CD.Endth/Mural.HIGD1B.NDUFA4L2, 7; p031, CD.Endth/Mural. HIGD1B.NDUFA4L2, 22; p032, CD.Endth/Mural.HIGD1B.NDUFA4L2, 0; p041,
CD.Endth/Mural.HIGD1B.NDUFA4L2, 8; p042, CD.Endth/Mural. HIGD1B.NDUFA4L2, 3; p048, CD.Endth/Mural.HIGD1B.NDUFA4L2, 9; p011,
CD.Endth/Ven.ACKR1.ADGRG6, 11; p014, CD.Endth/Ven.ACKR1.ADGRG6, 0; p016, CD.Endth/Ven.ACKR1.ADGRG6, 4; p018, CD.Endth/Ven.ACKR1.ADGRG6, 14; p019,
CD.Endth/Ven.ACKR1.ADGRG6, 2; p022, CD.Endth/Ven.ACKR1.ADGRG6, 4; p024, CD.Endth/Ven.ACKR1.ADGRG6, 7; p026, CD.Endth/Ven.ACKR1.ADGRG6, 4; p027,
CD.Endth/Ven.ACKR1.ADGRG6, 8; p031, CD.Endth/Ven.ACKR1.ADGRG6, 13; p032, CD.Endth/Ven.ACKR1.ADGRG6, 5; p041, CD.Endth/Ven.ACKR1.ADGRG6, 13; p042,
CD.Endth/Ven.ACKR1.ADGRG6, 4; p048, CD.Endth/Ven.ACKR1.ADGRG6, 3; p011, CD.Endth/Ven.ADGRG6.ACKR1, 18; p014, CD.Endth/Ven.ADGRG6.ACKR1, 0; p016,
CD.Endth/Ven.ADGRG6.ACKR1, 8; p018, CD.Endth/Ven.ADGRG6.ACKR1, 7; p019, CD.Endth/Ven.ADGRG6.ACKR1, 1; p022, CD.Endth/Ven.ADGRG6.ACKR1, 0; p024,
CD.Endth/Ven.ADGRG6.ACKR1, 0; p026, CD.Endth/Ven.ADGRG6.ACKR1, 0; p027, CD.Endth/Ven.ADGRG6.ACKR1, 6; p031, CD.Endth/Ven.ADGRG6.ACKR1, 1; p032,
CD.Endth/Ven.ADGRG6.ACKR1, 3; p041, CD.Endth/Ven.ADGRG6.ACKR1, 2; p042, CD.Endth/Ven.ADGRG6.ACKR1, 6; p048, CD.Endth/Ven.ADGRG6.ACKR1, 2; p011,
CD.Endth/Ven.CCL23.ACKR1, 6; p014, CD.Endth/Ven.CCL23.ACKR1, 1; p016, CD.Endth/Ven.CCL23.ACKR1, 1; p018, CD.Endth/Ven.CCL23.ACKR1, 58; p019,
CD.Endth/Ven.CCL23.ACKR1, 5; p022, CD.Endth/Ven.CCL23.ACKR1, 1; p024, CD.Endth/Ven.CCL23.ACKR1, 0; p026, CD.Endth/Ven.CCL23.ACKR1, 0; p027,
CD.Endth/Ven.CCL23.ACKR1, 4; p031, CD.Endth/Ven.CCL23.ACKR1, 0; p032, CD.Endth/Ven.CCL23.ACKR1, 0; p041, CD.Endth/Ven.CCL23.ACKR1, 2; p042,
CD.Endth/Ven.CCL23.ACKR1, 1; p048, CD.Endth/Ven.CCL23.ACKR1, 2; p011, CD.Endth/Ven.CPE.GALNT15, 1; p014, CD.Endth/Ven.CPE.GALNT15, 0; p016,
CD.Endth/Ven.CPE.GALNT15, 0; p018, CD.Endth/Ven.CPE.GALNT15, 0; p019, CD.Endth/Ven.CPE.GALNT15, 0; p022, CD.Endth/Ven.CPE.GALNT15, 0; p024,
CD.Endth/Ven.CPE.GALNT15, 0; p026, CD.Endth/Ven.CPE.GALNT15, 0; p027, CD.Endth/Ven.CPE.GALNT15, 1; p031, CD.Endth/Ven.CPE.GALNT15, 1; p032,
CD.Endth/Ven.CPE.GALNT15, 0; p041, CD.Endth/Ven.CPE.GALNT15, 1; p042, CD.Endth/Ven.CPE.GALNT15, 11; p048, CD.Endth/Ven.CPE.GALNT15, 18; p011,
CD.Endth/Ven.LAMP3.LIPG, 0; p014, CD.Endth/Ven.LAMP3.LIPG, 0; p016, CD.Endth/Ven.LAMP3.LIPG, 0; p018, CD.Endth/Ven.LAMP3.LIPG, 0; p019,
CD.Endth/Ven.LAMP3.LIPG, 0; p022, CD.Endth/Ven.LAMP3.LIPG, 4; p024, CD.Endth/Ven.LAMP3.LIPG, 1; p026, CD.Endth/Ven.LAMP3.LIPG, 0; p027,
CD.Endth/Ven.LAMP3.LIPG, 0; p031, CD.Endth/Ven.LAMP3.LIPG, 1; p032, CD.Endth/Ven.LAMP3.LIPG, 2; p041, CD.Endth/Ven.LAMP3.LIPG, 67; p042,
CD.Endth/Ven.LAMP3.LIPG, 0; p048, CD.Endth/Ven.LAMP3.LIPG, 0; p011, CD.Endth/Ven.POSTN.ACKR1, 2; p014, CD.Endth/Ven.POSTN.ACKR1, 0; p016,
CD.Endth/Ven.POSTN.ACKR1, 0; p018, CD.Endth/Ven.POSTN.ACKR1, 2; p019, CD.Endth/Ven.POSTN.ACKR1, 2; p022, CD.Endth/Ven.POSTN.ACKR1, 57; p024,
CD.Endth/Ven.POSTN.ACKR1, 0; p026, CD.Endth/Ven.POSTN.ACKR1, 0; p027, CD.Endth/Ven.POSTN.ACKR1, 2; p031, CD.Endth/Ven.POSTN.ACKR1, 0; p032,
CD.Endth/Ven.POSTN.ACKR1, 6; p041, CD.Endth/Ven.POSTN.ACKR1, 18; p042, CD.Endth/Ven.POSTN.ACKR1, 0; p048, CD.Endth/Ven.POSTN.ACKR1, 0; p011,
CD.Endth/Ven.POSTN.ADGRG6, 1; p014, CD.Endth/Ven.POSTN.ADGRG6, 2; p016, CD.Endth/Ven.POSTN.ADGRG6, 0; p018, CD.Endth/Ven.POSTN.ADGRG6, 0; p019,
CD.Endth/Ven.POSTN.ADGRG6, 0; p022, CD.Endth/Ven.POSTN.ADGRG6, 6; p024, CD.Endth/Ven.POSTN.ADGRG6, 41; p026, CD.Endth/Ven.POSTN.ADGRG6, 0; p027,
CD.Endth/Ven.POSTN.ADGRG6, 0; p031, CD.Endth/Ven.POSTN.ADGRG6, 5; p032, CD.Endth/Ven.POSTN.ADGRG6, 0; p041, CD.Endth/Ven.POSTN.ADGRG6, 4; p042,
CD.Endth/Ven.POSTN.ADGRG6, 0; p048, CD.Endth/Ven.POSTN.ADGRG6, 0; p011, CD.Enteroendocrine.NEUROG3.MLN, 5; p014, CD.Enteroendocrine.NEUROG3.MLN, 0;
p016, CD.Enteroendocrine.NEUROG3.MLN, 0; p018, CD.Enteroendocrine.NEUROG3.MLN, 2; p019, CD.Enteroendocrine.NEUROG3.MLN, 0; p022,
CD.Enteroendocrine.NEUROG3.MLN, 1; p024, CD.Enteroendocrine.NEUROG3.MLN, 0; p026, CD.Enteroendocrine.NEUROG3.MLN, 1; p027,
CD.Enteroendocrine.NEUROG3.MLN, 1; p031, CD.Enteroendocrine.NEUROG3.MLN, 6; p032, CD.Enteroendocrine.NEUROG3.MLN, 0; p041,
CD.Enteroendocrine.NEUROG3.MLN, 1; p042, CD.Enteroendocrine.NEUROG3.MLN, 0; p048, CD.Enteroendocrine.NEUROG3.MLN, 0; p011,
CD.Enteroendocrine.ONECUT3.CCK, 3; p014, CD.Enteroendocrine.ONECUT3.CCK, 0; p016, CD.Enteroendocrine.ONECUT3.CCK, 0; p018,
CD.Enteroendocrine.ONECUT3.CCK, 9; p019, CD.Enteroendocrine.ONECUT3.CCK, 2; p022, CD.Enteroendocrine.ONECUT3.CCK, 2; p024,
CD.Enteroendocrine.ONECUT3.CCK, 1; p026, CD.Enteroendocrine.ONECUT3.CCK, 6; p027, CD.Enteroendocrine.ONECUT3.CCK, 1; p031,
CD.Enteroendocrine.ONECUT3.CCK, 6; p032, CD.Enteroendocrine.ONECUT3.CCK, 3; p041, CD.Enteroendocrine.ONECUT3.CCK, 0; p042,
CD.Enteroendocrine.ONECUT3.CCK, 0; p048, CD.Enteroendocrine.ONECUT3.CCK, 0; p011, CD.Enteroendocrine.TFPI2.TPH1, 12; p014, CD.Enteroendocrine.TFPI2.TPH1, 8;
p016, CD.Enteroendocrine.TFPI2.TPH1, 0; p018, CD.Enteroendocrine.TFPI2.TPH1, 14; p019, CD.Enteroendocrine. TFPI2.TPH1, 0; p022, CD.Enteroendocrine.TFPI2.TPH1,
4; p024, CD.Enteroendocrine.TFPI2.TPH1, 1; p026, CD.Enteroendocrine.TFPI2.TPH1, 11; p027, CD.Enteroendocrine.TFPI2.TPH1, 1; p031,
CD.Enteroendocrine. TFPI2.TPH1, 7; p032, CD.Enteroendocrine.TFPI2.TPH1, 1; p041, CD.Enteroendocrine.TFPI2.TPH1, 3; p042, CD.Enteroendocrine.TFPI2.TPH1, 0; p048,
CD.Enteroendocrine.TFPI2.TPH1, 2; p011, CD.Enteroendocrine.TPH1.CHGA, 7; p014, CD.Enteroendocrine.TPH1.CHGA, 4; p016, CD.Enteroendocrine.TPH1.CHGA, 2; p018,
CD.Enteroendocrine.TPH1.CHGA, 4; p019, CD.Enteroendocrine.TPH1.CHGA, 0; p022, CD.Enteroendocrine.TPH1.CHGA, 3; p024, CD.Enteroendocrine.TPH1.CHGA, 2; p026,
CD.Enteroendocrine.TPH1.CHGA, 2; p027, CD.Enteroendocrine.TPH1.CHGA, 4; p031, CD.Enteroendocrine.TPH1.CHGA, 5; p032, CD.Enteroendocrine.TPH1.CHGA, 2; p041,
CD.Enteroendocrine.TPH1.CHGA, 2; p042, CD.Enteroendocrine.TPH1.CHGA, 0; p048, CD.Enteroendocrine.TPH1.CHGA, 12; p011, CD.EpithStem.LINC00176.RPS4Y1, 0;
p014, CD.EpithStem.LINC00176.RPS4Y1, 0; p016, CD.EpithStem.LINC00176.RPS4Y1, 0; p018, CD.EpithStem.LINC00176.RPS4Y1, 7; p019,
CD.EpithStem.LINC00176.RPS4Y1, 6; p022, CD.EpithStem.LINC00176.RPS4Y1, 0; p024, CD.EpithStem.LINC00176.RPS4Y1, 0; p026, CD.EpithStem.LINC00176.RPS4Y1, 1;
p027, CD.EpithStem.LINC00176.RPS4Y1, 66; p031, CD.EpithStem.LINC00176.RPS4Y1, 0; p032, CD.EpithStem.LINC00176.RPS4Y1, 0; p041,
CD.EpithStem.LINC00176.RPS4Y1, 0; p042, CD.EpithStem.LINC00176.RPS4Y1, 0; p048, CD.EpithStem.LINC00176.RPS4Y1, 1; p011, CD.Fibro.ADAMDEC1.C7, 0; p014,
CD.Fibro.ADAMDEC1.C7, 5; p016, CD.Fibro.ADAMDEC1.C7, 0; p018, CD.Fibro.ADAMDEC1.C7, 0; p019, CD.Fibro.ADAMDEC1.C7, 3; p022, CD.Fibro.ADAMDEC1.C7, 0; p024,
CD.Fibro.ADAMDEC1.C7, 0; p026, CD.Fibro.ADAMDEC1.C7, 9; p027, CD.Fibro.ADAMDEC1.C7, 3; p031, CD.Fibro.ADAMDEC1.C7, 19; p032, CD.Fibro.ADAMDEC1.C7, 0; p041,
CD.Fibro.ADAMDEC1.C7, 0; p042, CD.Fibro.ADAMDEC1.C7, 472; p048, CD.Fibro.ADAMDEC1.C7, 133; p011, CD.Fibro.AGT.F3, 0; p014, CD.Fibro.AGT.F3, 1; p016,
CD.Fibro.AGT.F3, 0; p018, CD.Fibro.AGT.F3, 72; p019, CD.Fibro.AGT.F3, 3; p022, CD.Fibro.AGT.F3, 9; p024, CD.Fibro.AGT.F3, 15; p026, CD.Fibro.AGT.F3, 8; p027,
CD.Fibro.AGT.F3, 6; p031, CD.Fibro.AGT.F3, 4; p032, CD.Fibro.AGT.F3, 7; p041, CD.Fibro.AGT.F3, 7; p042, CD.Fibro.AGT.F3, 5; p048, CD.Fibro.AGT.F3, 0; p011,
CD.Fibro.CA12.SFRP2, 0; p014, CD.Fibro.CA12.SFRP2, 0; p016, CD.Fibro.CA12.SFRP2, 0; p018, CD.Fibro.CA12.SFRP2, 0; p019, CD.Fibro.CA12.SFRP2, 0; p022,
CD.Fibro.CA12.SFRP2, 55; p024, CD.Fibro.CA12.SFRP2, 0; p026, CD.Fibro.CA12.SFRP2, 0; p027, CD.Fibro.CA12.SFRP2, 0; p031, CD.Fibro.CA12.SFRP2, 0; p032,
CD.Fibro.CA12.SFRP2, 0; p041, CD.Fibro.CA12.SFRP2, 0; p042, CD.Fibro.CA12.SFRP2, 0; p048, CD.Fibro.CA12.SFRP2, 0; p011, CD.Fibro.CCL13.CXCL6, 0; p014,
CD.Fibro.CCL13.CXCL6, 2; p016, CD.Fibro.CCL13.CXCL6, 1; p018, CD.Fibro.CCL13.CXCL6, 1; p019, CD.Fibro.CCL13.CXCL6, 0; p022, CD.Fibro.CCL13.CXCL6, 0; p024,
CD.Fibro.CCL13.CXCL6, 209; p026, CD.Fibro.CCL13.CXCL6, 0; p027, CD.Fibro.CCL13.CXCL6, 0; p031, CD.Fibro.CCL13.CXCL6, 0; p032, CD.Fibro.CCL13.CXCL6, 0; p041,
CD.Fibro.CCL13.CXCL6, 2; p042, CD.Fibro.CCL13.CXCL6, 1; p048, CD.Fibro.CCL13.CXCL6, 0; p011, CD.Fibro.CCL19.IRF7, 0; p014, CD.Fibro.CCL19.IRF7, 0; p016,
CD.Fibro.CCL19.IRF7, 0; p018, CD.Fibro.CCL19.IRF7, 3; p019, CD.Fibro.CCL19.IRF7, 2; p022, CD.Fibro.CCL19.IRF7, 1; p024, CD.Fibro.CCL19.IRF7, 1; p026,
CD.Fibro.CCL19.IRF7, 26; p027, CD.Fibro.CCL19.IRF7, 4; p031, CD.Fibro.CCL19.IRF7, 0; p032, CD.Fibro.CCL19.IRF7, 0; p041, CD.Fibro.CCL19.IRF7, 4; p042,
CD.Fibro.CCL19.IRF7, 5; p048, CD.Fibro.CCL19.IRF7, 2; p011, CD.Fibro.CCL21.CCL19, 0; p014, CD.Fibro.CCL21.CCL19, 0; p016, CD.Fibro.CCL21.CCL19, 1; p018,
CD.Fibro.CCL21.CCL19, 27; p019, CD.Fibro.CCL21.CCL19, 42; p022, CD.Fibro.CCL21.CCL19, 2; p024, CD.Fibro.CCL21.CCL19, 0; p026, CD.Fibro.CCL21.CCL19, 21; p027,
CD.Fibro.CCL21.CCL19, 6; p031, CD.Fibro.CCL21.CCL19, 1; p032, CD.Fibro.CCL21.CCL19, 4; p041, CD.Fibro.CCL21.CCL19, 20; p042, CD.Fibro.CCL21.CCL19, 4; p048,
CD.Fibro.CCL21.CCL19, 0; p011, CD.Fibro.CD24.IGHG4, 0; p014, CD.Fibro.CD24.IGHG4, 0; p016, CD.Fibro.CD24.IGHG4, 0; p018, CD.Fibro.CD24.IGHG4, 0; p019,
CD.Fibro.CD24.IGHG4, 0; p022, CD.Fibro.CD24.IGHG4, 0; p024, CD.Fibro.CD24.IGHG4, 0; p026, CD.Fibro.CD24.IGHG4, 0; p027, CD.Fibro.CD24.IGHG4, 0; p031,
CD.Fibro.CD24.IGHG4, 0; p032, CD.Fibro.CD24.IGHG4, 36; p041, CD.Fibro.CD24.IGHG4, 1; p042, CD.Fibro.CD24.IGHG4, 0; p048, CD.Fibro.CD24.IGHG4, 0; p011,
CD.Fibro.CHI3L1.CXCL3, 0; p014, CD.Fibro.CHI3L1.CXCL3, 2; p016, CD.Fibro.CHI3L1.CXCL3, 0; p018, CD.Fibro.CHI3L1.CXCL3, 7; p019, CD.Fibro.CHI3L1.CXCL3, 0; p022,
CD.Fibro.CHI3L1.CXCL3, 6; p024, CD.Fibro.CHI3L1.CXCL3, 27; p026, CD.Fibro.CHI3L1.CXCL3, 5; p027, CD.Fibro.CHI3L1.CXCL3, 2; p031, CD.Fibro.CHI3L1.CXCL3, 1; p032,
CD.Fibro.CHI3L1.CXCL3, 5; p041, CD.Fibro.CHI3L1.CXCL3, 3; p042, CD.Fibro.CHI3L1.CXCL3, 0; p048, CD.Fibro.CHI3L1.CXCL3, 0; p011, CD.Fibro.COL6A5.CXCL6, 0; p014,
CD.Fibro.COL6A5.CXCL6, 0; p016, CD.Fibro.COL6A5.CXCL6, 0; p018, CD.Fibro.COL6A5.CXCL6, 0; p019, CD.Fibro.COL6A5.CXCL6, 0; p022, CD.Fibro.COL6A5.CXCL6, 0; p024,
CD.Fibro.COL6A5.CXCL6, 0; p026, CD.Fibro.COL6A5.CXCL6, 0; p027, CD.Fibro.COL6A5.CXCL6, 3; p031, CD.Fibro.COL6A5.CXCL6, 135; p032, CD.Fibro.COL6A5.CXCL6, 0;
p041, CD.Fibro.COL6A5.CXCL6, 0; p042, CD.Fibro.COL6A5.CXCL6, 0; p048, CD.Fibro.COL6A5.CXCL6, 0; p011, CD.Fibro.COL6A5.SLITRK6, 0; p014,
CD.Fibro.COL6A5.SLITRK6, 0; p016, CD.Fibro.COL6A5.SLITRK6, 0; p018, CD.Fibro.COL6A5.SLITRK6, 0; p019, CD.Fibro.COL6A5.SLITRK6, 0; p022,
CD.Fibro.COL6A5.SLITRK6, 0; p024, CD.Fibro.COL6A5.SLITRK6, 1; p026, CD.Fibro.COL6A5.SLITRK6, 0; p027, CD.Fibro.COL6A5.SLITRK6, 2; p031,
CD.Fibro.COL6A5.SLITRK6, 18; p032, CD.Fibro.COL6A5.SLITRK6, 1; p041, CD.Fibro.COL6A5.SLITRK6, 0; p042, CD.Fibro.COL6A5.SLITRK6, 0; p048,
CD.Fibro.COL6A5.SLITRK6, 0; p011, CD.Fibro.CPM.HSD17B2, 0; p014, CD.Fibro.CPM.HSD17B2, 0; p016, CD.Fibro.CPM.HSD17B2, 42; p018, CD.Fibro.CPM.HSD17B2, 0;
p019, CD.Fibro.CPM.HSD17B2, 0; p022, CD.Fibro.CPM.HSD17B2, 0; p024, CD.Fibro.CPM.HSD17B2, 0; p026, CD.Fibro.CPM.HSD17B2, 0; p027, CD.Fibro.CPM.HSD17B2, 0;
p031, CD.Fibro.CPM.HSD17B2, 0; p032, CD.Fibro.CPM.HSD17B2, 0; p041, CD.Fibro.CPM.HSD17B2, 0; p042, CD.Fibro.CPM.HSD17B2, 0; p048, CD.Fibro.CPM.HSD17B2, 0;
p011, CD.Fibro.CXCL14.DEFA5, 0; p014, CD.Fibro.CXCL14.DEFA5, 0; p016, CD.Fibro.CXCL14.DEFA5, 0; p018, CD.Fibro.CXCL14.DEFA5, 93; p019, CD.Fibro.CXCL14.DEFA5,
2; p022, CD.Fibro.CXCL14.DEFA5, 1; p024, CD.Fibro.CXCL14.DEFA5, 0; p026, CD.Fibro.CXCL14.DEFA5, 0; p027, CD.Fibro.CXCL14.DEFA5, 0; p031, CD.Fibro.CXCL14.DEFA5,
1; p032, CD.Fibro.CXCL14.DEFA5, 0; p041, CD.Fibro.CXCL14.DEFA5, 0; p042, CD.Fibro.CXCL14.DEFA5, 0; p048, CD.Fibro.CXCL14.DEFA5, 0; p011, CD.Fibro.DEFA6.DEFA5,
0; p014, CD.Fibro.DEFA6.DEFA5, 0; p016, CD.Fibro.DEFA6.DEFA5, 0; p018, CD.Fibro.DEFA6.DEFA5, 82; p019, CD.Fibro.DEFA6.DEFA5, 3; p022, CD.Fibro.DEFA6.DEFA5, 5;
p024, CD.Fibro.DEFA6.DEFA5, 1; p026, CD.Fibro.DEFA6.DEFA5, 0; p027, CD.Fibro.DEFA6.DEFA5, 0; p031, CD.Fibro.DEFA6.DEFA5, 1; p032, CD.Fibro.DEFA6.DEFA5, 0;
p041, CD.Fibro.DEFA6.DEFA5, 1; p042, CD.Fibro.DEFA6.DEFA5, 0; p048, CD.Fibro.DEFA6.DEFA5, 0; p011, CD.Fibro.F3.SOX6, 0; p014, CD.Fibro.F3.SOX6, 6; p016,
CD.Fibro.F3.SOX6, 0; p018, CD.Fibro.F3.SOX6, 0; p019, CD.Fibro.F3.SOX6, 2; p022, CD.Fibro.F3.SOX6, 5; p024, CD.Fibro.F3.SOX6, 11; p026, CD.Fibro.F3.SOX6, 2; p027,
CD.Fibro.F3.SOX6, 1; p031, CD.Fibro.F3.SOX6, 7; p032, CD.Fibro.F3.SOX6, 10; p041, CD.Fibro.F3.SOX6, 5; p042, CD.Fibro.F3.SOX6, 6; p048, CD.Fibro.F3.SOX6, 3; p011,
CD.Fibro.FIGF.MMP11, 0; p014, CD.Fibro.FIGF.MMP11, 0; p016, CD.Fibro.FIGF.MMP11, 0; p018, CD.Fibro.FIGF.MMP11, 0; p019, CD.Fibro.FIGF.MMP11, 1; p022,
CD.Fibro.FIGF.MMP11, 0; p024, CD.Fibro.FIGF.MMP11, 0; p026, CD.Fibro.FIGF.MMP11, 2; p027, CD.Fibro.FIGF.MMP11, 1; p031, CD.Fibro.FIGF.MMP11, 0; p032,
CD.Fibro.FIGF.MMP11, 0; p041, CD.Fibro.FIGF.MMP11, 0; p042, CD.Fibro.FIGF.MMP11, 152; p048, CD.Fibro.FIGF.MMP11, 19; p011, CD.Fibro.GDF15.EGR1, 0; p014,
CD.Fibro.GDF15.EGR1, 2; p016, CD.Fibro.GDF15.EGR1, 0; p018, CD.Fibro.GDF15.EGR1, 0; p019, CD.Fibro.GDF15.EGR1, 0; p022, CD.Fibro.GDF15.EGR1, 13; p024,
CD.Fibro.GDF15.EGR1, 0; p026, CD.Fibro.GDF15.EGR1, 2; p027, CD.Fibro.GDF15.EGR1, 0; p031, CD.Fibro.GDF15.EGR1, 0; p032, CD.Fibro.GDF15.EGR1, 13; p041,
CD.Fibro.GDF15.EGR1, 0; p042, CD.Fibro.GDF15.EGR1, 0; p048, CD.Fibro.GDF15.EGR1, 0; p011, CD.Fibro.HIGD1B.RGS5, 42; p014, CD.Fibro.HIGD1B.RGS5, 18; p016,
CD.Fibro. HIGD1B.RGS5, 14; p018, CD.Fibro.HIGD1B.RGS5, 31; p019, CD.Fibro.HIGD1B.RGS5, 2; p022, CD.Fibro.HIGD1B.RGS5, 101; p024, CD.Fibro.HIGD1B.RGS5, 27; p026,
CD.Fibro.HIGD1B.RGS5, 19; p027, CD.Fibro.HIGD1B.RGS5, 15; p031, CD.Fibro.HIGD1B.RGS5, 10; p032, CD.Fibro.HIGD1B.RGS5, 6; p041, CD.Fibro.HIGD1B.RGS5, 47; p042,
CD.Fibro. HIGD1B.RGS5, 9; p048, CD.Fibro.HIGD1B.RGS5, 27; p011, CD.Fibro.IFI6.IFI44L, 0; p014, CD.Fibro.IFI6.IFI44L, 1; p016, CD.Fibro.IFI6.IFI44L, 0; p018,
CD.Fibro.IFI6.IFI44L, 0; p019, CD.Fibro.IFI6.IFI44L, 5; p022, CD.Fibro.IFI6.IFI44L, 0; p024, CD.Fibro.IFI6.IFI44L, 0; p026, CD.Fibro.IFI6.IFI44L, 44; p027,
CD.Fibro.IFI6.IFI44L, 2; p031, CD.Fibro.IFI6.IFI44L, 0; p032, CD.Fibro.IFI6.IFI44L, 2; p041, CD.Fibro.IFI6.IFI44L, 0; p042, CD.Fibro.IFI6.IFI44L, 9; p048,
CD.Fibro.IFI6.IFI44L, 1; p011, CD.Fibro.IGF2.CXCL6, 0; p014, CD.Fibro.IGF2.CXCL6, 0; p016, CD.Fibro.IGF2.CXCL6, 0; p018, CD.Fibro.IGF2.CXCL6, 0; p019,
CD.Fibro.IGF2.CXCL6, 1; p022, CD.Fibro.IGF2.CXCL6, 35; p024, CD.Fibro.IGF2.CXCL6, 0; p026, CD.Fibro.IGF2.CXCL6, 0; p027, CD.Fibro.IGF2.CXCL6, 0; p031,
CD.Fibro.IGF2.CXCL6, 0; p032, CD.Fibro.IGF2.CXCL6, 0; p041, CD.Fibro.IGF2.CXCL6, 0; p042, CD.Fibro.IGF2.CXCL6, 0; p048, CD.Fibro.IGF2.CXCL6, 0; p011,
CD.Fibro.LY6H.PAPPA2, 0; p014, CD.Fibro.LY6H.PAPPA2, 0; p016, CD.Fibro.LY6H.PAPPA2, 0; p018, CD.Fibro.LY6H.PAPPA2, 1; p019, CD.Fibro.LY6H.PAPPA2, 1; p022,
CD.Fibro.LY6H.PAPPA2, 19; p024, CD.Fibro.LY6H.PAPPA2, 38; p026, CD.Fibro.LY6H.PAPPA2, 5; p027, CD.Fibro.LY6H.PAPPA2, 1; p031, CD.Fibro.LY6H.PAPPA2, 1; p032,
CD.Fibro.LY6H.PAPPA2, 13; p041, CD.Fibro.LY6H.PAPPA2, 30; p042, CD.Fibro.LY6H.PAPPA2, 0; p048, CD.Fibro.LY6H.PAPPA2, 1; p011, CD.Fibro.LY6H.PCSK6, 0; p014,
CD.Fibro.LY6H.PCSK6, 1; p016, CD.Fibro.LY6H.PCSK6, 0; p018, CD.Fibro.LY6H.PCSK6, 0; p019, CD.Fibro.LY6H.PCSK6, 0; p022, CD.Fibro.LY6H.PCSK6, 0; p024,
CD.Fibro.LY6H.PCSK6, 0; p026, CD.Fibro.LY6H.PCSK6, 1; p027, CD.Fibro.LY6H.PCSK6, 0; p031, CD.Fibro.LY6H.PCSK6, 52; p032, CD.Fibro.LY6H.PCSK6, 0; p041,
CD.Fibro.LY6H.PCSK6, 0; p042, CD.Fibro.LY6H.PCSK6, 0; p048, CD.Fibro.LY6H.PCSK6, 1; p011, CD.Fibro.MT-ND4L.NEAT1, 0; p014, CD.Fibro.MT-ND4L.NEAT1, 9; p016,
CD.Fibro.MT-ND4L.NEAT1, 0; p018, CD.Fibro.MT-ND4L.NEAT1, 0; p019, CD.Fibro.MT-ND4L.NEAT1, 1; p022, CD.Fibro.MT-ND4L.NEAT1, 5; p024, CD.Fibro.MT-
ND4L.NEAT1, 12; p026, CD.Fibro. MT-ND4L.NEAT1, 2; p027, CD.Fibro.MT-ND4L.NEAT1, 0; p031, CD.Fibro.MT-ND4L.NEAT1, 11; p032, CD.Fibro. MT-ND4L.NEAT1, 7; p041,
CD.Fibro.MT-ND4L.NEAT1, 4; p042, CD.Fibro.MT-ND4L.NEAT1, 2; p048, CD.Fibro.MT-ND4L.NEAT1, 0; p011, CD.Fibro.NPY.HSD17B2, 0; p014, CD.Fibro.NPY.HSD17B2, 1;
p016, CD.Fibro.NPY.HSD17B2, 78; p018, CD.Fibro.NPY.HSD17B2, 0; p019, CD.Fibro.NPY.HSD17B2, 0; p022, CD.Fibro.NPY.HSD17B2, 0; p024, CD.Fibro.NPY.HSD17B2, 0;
p026, CD.Fibro.NPY.HSD17B2, 0; p027, CD.Fibro.NPY.HSD17B2, 0; p031, CD.Fibro.NPY.HSD17B2, 0; p032, CD.Fibro.NPY.HSD17B2, 0; p041, CD.Fibro.NPY.HSD17B2, 0;
p042, CD.Fibro.NPY.HSD17B2, 0; p048, CD.Fibro.NPY.HSD17B2, 0; p011, CD.Fibro.OGN.C1QTNF3, 0; p014, CD.Fibro.OGN.C1QTNF3, 4; p016, CD.Fibro.OGN.C1QTNF3, 13;
p018, CD.Fibro.OGN.C1QTNF3, 0; p019, CD.Fibro.OGN.C1QTNF3, 0; p022, CD.Fibro.OGN.C1QTNF3, 3; p024, CD.Fibro.OGN.C1QTNF3, 5; p026, CD.Fibro.OGN.C1QTNF3, 0;
p027, CD.Fibro.OGN.C1QTNF3, 1; p031, CD.Fibro.OGN.C1QTNF3, 1; p032, CD.Fibro.OGN.C1QTNF3, 0; p041, CD.Fibro.OGN.C1QTNF3, 4; p042, CD.Fibro.OGN.C1QTNF3, 0;
p048, CD.Fibro.OGN.C1QTNF3, 1; p011, CD.Fibro.PCDH18.APOE, 0; p014, CD.Fibro.PCDH18.APOE, 0; p016, CD.Fibro.PCDH18.APOE, 0; p018, CD.Fibro.PCDH18.APOE, 0;
p019, CD.Fibro. PCDH18.APOE, 3; p022, CD.Fibro.PCDH18.APOE, 5; p024, CD.Fibro.PCDH18.APOE, 38; p026, CD.Fibro.PCDH18.APOE, 0; p027, CD.Fibro.PCDH18.APOE, 1;
p031, CD.Fibro.PCDH18.APOE, 6; p032, CD.Fibro.PCDH18.APOE, 5; p041, CD.Fibro.PCDH18.APOE, 1; p042, CD.Fibro.PCDH18.APOE, 0; p048, CD.Fibro.PCDH18.APOE, 2;
p011, CD.Fibro.PCSK6.FIGF, 0; p014, CD.Fibro.PCSK6.FIGF, 0; p016, CD.Fibro.PCSK6.FIGF, 48; p018, CD.Fibro.PCSK6.FIGF, 0; p019, CD.Fibro.PCSK6.FIGF, 0; p022,
CD.Fibro.PCSK6.FIGF, 0; p024, CD.Fibro.PCSK6.FIGF, 0; p026, CD.Fibro.PCSK6.FIGF, 0; p027, CD.Fibro.PCSK6.FIGF, 0; p031, CD.Fibro.PCSK6.FIGF, 1; p032,
CD.Fibro.PCSK6.FIGF, 0; p041, CD.Fibro.PCSK6.FIGF, 0; p042, CD.Fibro.PCSK6.FIGF, 0; p048, CD.Fibro.PCSK6.FIGF, 0; p011, CD.Fibro.PHGDH.STMN2, 0; p014,
CD.Fibro.PHGDH.STMN2, 0; p016, CD.Fibro.PHGDH.STMN2, 0; p018, CD.Fibro.PHGDH.STMN2, 79; p019, CD.Fibro.PHGDH.STMN2, 0; p022, CD.Fibro.PHGDH.STMN2, 1;
p024, CD.Fibro.PHGDH.STMN2, 0; p026, CD.Fibro.PHGDH.STMN2, 0; p027, CD.Fibro.PHGDH.STMN2, 0; p031, CD.Fibro.PHGDH.STMN2, 0; p032, CD.Fibro.PHGDH.STMN2,
0; p041, CD.Fibro.PHGDH.STMN2, 0; p042, CD.Fibro.PHGDH.STMN2, 0; p048, CD.Fibro.PHGDH.STMN2, 0; p011, CD.Fibro.PRELP.OGN, 0; p014, CD.Fibro.PRELP.OGN, 1;
p016, CD.Fibro.PRELP.OGN, 0; p018, CD.Fibro.PRELP.OGN, 0; p019, CD.Fibro.PRELP.OGN, 1; p022, CD.Fibro.PRELP.OGN, 2; p024, CD.Fibro.PRELP.OGN, 3; p026,
CD.Fibro.PRELP.OGN, 1; p027, CD.Fibro.PRELP.OGN, 1; p031, CD.Fibro.PRELP.OGN, 1; p032, CD.Fibro.PRELP.OGN, 3; p041, CD.Fibro.PRELP.OGN, 0; p042,
CD.Fibro.PRELP.OGN, 11; p048, CD.Fibro.PRELP.OGN, 2; p011, CD.Fibro.RAMP1.IGHG1, 0; p014, CD.Fibro.RAMP1.IGHG1, 0; p016, CD.Fibro.RAMP1.IGHG1, 1; p018,
CD.Fibro.RAMP1.IGHG1, 1; p019, CD.Fibro.RAMP1.IGHG1, 0; p022, CD.Fibro.RAMP1.IGHG1, 40; p024, CD.Fibro.RAMP1.IGHG1, 0; p026, CD.Fibro.RAMP1.IGHG1, 0; p027,
CD.Fibro.RAMP1.IGHG1, 0; p031, CD.Fibro.RAMP1.IGHG1, 0; p032, CD.Fibro.RAMP1.IGHG1, 0; p041, CD.Fibro.RAMP1.IGHG1, 3; p042, CD.Fibro.RAMP1.IGHG1, 0; p048,
CD.Fibro.RAMP1.IGHG1, 0; p011, CD.Fibro.RERGL.NTRK2, 0; p014, CD.Fibro.RERGL.NTRK2, 6; p016, CD.Fibro.RERGL.NTRK2, 9; p018, CD.Fibro.RERGL.NTRK2, 74; p019,
CD.Fibro.RERGL.NTRK2, 6; p022, CD.Fibro.RERGL.NTRK2, 45; p024, CD.Fibro.RERGL.NTRK2, 8; p026, CD.Fibro.RERGL.NTRK2, 12; p027, CD.Fibro.RERGL.NTRK2, 5; p031,
CD.Fibro.RERGL.NTRK2, 5; p032, CD.Fibro.RERGL.NTRK2, 0; p041, CD.Fibro.RERGL.NTRK2, 1; p042, CD.Fibro.RERGL.NTRK2, 3; p048, CD.Fibro.RERGL.NTRK2, 4; p011,
CD.Fibro.RPS4Y1.DEFA5, 0; p014, CD.Fibro.RPS4Y1.DEFA5, 0; p016, CD.Fibro.RPS4Y1.DEFA5, 0; p018, CD.Fibro.RPS4Y1.DEFA5, 81; p019, CD.Fibro.RPS4Y1.DEFA5, 3;
p022, CD.Fibro.RPS4Y1.DEFA5, 1; p024, CD.Fibro.RPS4Y1.DEFA5, 0; p026, CD.Fibro.RPS4Y1.DEFA5, 0; p027, CD.Fibro.RPS4Y1.DEFA5, 0; p031, CD.Fibro.RPS4Y1.DEFA5,
0; p032, CD.Fibro.RPS4Y1.DEFA5, 1; p041, CD.Fibro.RPS4Y1.DEFA5, 0; p042, CD.Fibro.RPS4Y1.DEFA5, 0; p048, CD.Fibro.RPS4Y1.DEFA5, 0; p011, CD.Fibro.STMN2.FABP5,
0; p014, CD.Fibro.STMN2.FABP5, 0; p016, CD.Fibro.STMN2.FABP5, 0; p018, CD.Fibro.STMN2.FABP5, 57; p019, CD.Fibro.STMN2.FABP5, 5; p022, CD.Fibro.STMN2.FABP5,
0; p024, CD.Fibro.STMN2.FABP5, 0; p026, CD.Fibro.STMN2.FABP5, 1; p027, CD.Fibro.STMN2.FABP5, 0; p031, CD.Fibro.STMN2.FABP5, 0; p032, CD.Fibro.STMN2.FABP5, 0;
p041, CD.Fibro.STMN2.FABP5, 0; p042, CD.Fibro.STMN2.FABP5, 0; p048, CD.Fibro.STMN2.FABP5, 0; p011, CD.Fibro.TFPI2.CCL13, 0; p014, CD.Fibro.TFPI2.CCL13, 0; p016,
CD.Fibro.TFPI2.CCL13, 369; p018, CD.Fibro.TFPI2.CCL13, 2; p019, CD.Fibro.TFPI2.CCL13, 0; p022, CD.Fibro.TFPI2.CCL13, 16; p024, CD.Fibro.TFPI2.CCL13, 9; p026,
CD.Fibro.TFPI2.CCL13, 1; p027, CD.Fibro.TFPI2.CCL13, 2; p031, CD.Fibro.TFPI2.CCL13, 8; p032, CD.Fibro.TFPI2.CCL13, 0; p041, CD.Fibro.TFPI2.CCL13, 74; p042,
CD.Fibro.TFPI2.CCL13, 2; p048, CD.Fibro.TFPI2.CCL13, 8; p011, CD.Fibro.TNFRSF11B.TFPI2, 0; p014, CD.Fibro.TNFRSF11B.TFP12, 0; p016, CD.Fibro.TNFRSF11B.TFPI2, 0;
p018, CD.Fibro.TNFRSF11B.TFPI2, 0; p019, CD.Fibro.TNFRSF11B.TFPI2, 0; p022, CD.Fibro.TNFRSF11B.TFPI2, 0; p024, CD.Fibro.TNFRSF11B.TFPI2, 0; p026,
CD.Fibro.TNFRSF11B.TFPI2, 0; p027, CD.Fibro.TNFRSF11B.TFPI2, 0; p031, CD.Fibro.TNFRSF11B.TFPI2, 0; p032, CD.Fibro.TNFRSF11B.TFPI2, 0; p041,
CD.Fibro.TNFRSF11B.TFPI2, 49; p042, CD.Fibro.TNFRSF11B.TFPI2, 0; p048, CD.Fibro.TNFRSF11B.TFPI2, 0; p011, CD.Fibro.TNFSF11.CD24, 0; p014,
CD.Fibro.TNFSF11.CD24, 0; p016, CD.Fibro.TNFSF11.CD24, 8; p018, CD.Fibro.TNFSF11.CD24, 142; p019, CD.Fibro.TNFSF11.CD24, 30; p022, CD.Fibro.TNFSF11.CD24, 2;
p024, CD.Fibro.TNFSF11.CD24, 3; p026, CD.Fibro.TNFSF11.CD24, 49; p027, CD.Fibro.TNFSF11.CD24, 13; p031, CD.Fibro.TNFSF11.CD24, 13; p032,
CD.Fibro.TNFSF11.CD24, 9; p041, CD.Fibro.TNFSF11.CD24, 4; p042, CD.Fibro.TNFSF11.CD24, 7; p048, CD.Fibro.TNFSF11.CD24, 0; p011, CD.Fibro.WFDC1.PTN, 0; p014,
CD.Fibro.WFDC1.PTN, 0; p016, CD.Fibro.WFDC1.PTN, 0; p018, CD.Fibro.WFDC1.PTN, 0; p019, CD.Fibro.WFDC1.PTN, 2; p022, CD.Fibro.WFDC1.PTN, 0; p024,
CD.Fibro.WFDC1.PTN, 1; p026, CD.Fibro.WFDC1.PTN, 0; p027, CD.Fibro.WFDC1.PTN, 10; p031, CD.Fibro.WFDC1.PTN, 6; p032, CD.Fibro.WFDC1.PTN, 0; p041,
CD.Fibro.WFDC1.PTN, 0; p042, CD.Fibro.WFDC1.PTN, 19; p048, CD.Fibro.WFDC1.PTN, 18; p011, CD.Fibro/fDC.FDCSP.CXCL13, 0; p014, CD.Fibro/fDC.FDCSP.CXCL13, 0;
p016, CD.Fibro/fDC.FDCSP.CXCL13, 5; p018, CD.Fibro/fDC.FDCSP.CXCL13, 9; p019, CD.Fibro/fDC.FDCSP.CXCL13, 43; p022, CD.Fibro/fDC.FDCSP.CXCL13, 1; p024,
CD.Fibro/fDC.FDCSP.CXCL13, 1; p026, CD.Fibro/fDC.FDCSP.CXCL13, 7; p027, CD.Fibro/fDC.FDCSP.CXCL13, 0; p031, CD.Fibro/fDC.FDCSP.CXCL13, 0; p032,
CD.Fibro/fDC.FDCSP.CXCL13, 0; p041, CD.Fibro/fDC.FDCSP.CXCL13, 2; p042, CD.Fibro/fDC.FDCSP.CXCL13, 2; p048, CD.Fibro/fDC.FDCSP.CXCL13, 0; p011,
CD.Glial.COL28A1.SPP1, 0; p014, CD.Glial.COL28A1.SPP1, 35; p016, CD.Glial.COL28A1.SPP1, 0; p018, CD.Glial.COL28A1.SPP1, 0; p019, CD.Glial.COL28A1.SPP1, 0; p022,
CD.Glial.COL28A1.SPP1, 0; p024, CD.Glial.COL28A1.SPP1, 0; p026, CD.Glial.COL28A1.SPP1, 0; p027, CD.Glial.COL28A1.SPP1, 0; p031, CD.Glial.COL28A1.SPP1, 0; p032,
CD.Glial.COL28A1.SPP1, 0; p041, CD.Glial.COL28A1.SPP1, 0; p042, CD.Glial.COL28A1.SPP1, 0; p048, CD.Glial.COL28A1.SPP1, 0; p011, CD.Glial.S100B.PLP1, 3; p014,
CD.Glial.S100B.PLP1, 1; p016, CD.Glial.S100B.PLP1, 0; p018, CD.Glial.S100B.PLP1, 16; p019, CD.Glial.S100B.PLP1, 0; p022, CD.Glial.S100B.PLP1, 3; p024,
CD.Glial.S100B.PLP1, 2; p026, CD.Glial.S100B.PLP1, 8; p027, CD.Glial.S100B.PLP1, 4; p031, CD.Glial.S100B.PLP1, 9; p032, CD.Glial.S100B.PLP1, 0; p041,
CD.Glial.S100B.PLP1, 8; p042, CD.Glial.S100B.PLP1, 11; p048, CD.Glial.S100B.PLP1, 4; p011, CD.Glial.S100B.SPP1, 4; p014, CD.Glial.S100B.SPP1, 0; p016,
CD.Glial.S100B.SPP1, 9; p018, CD.Glial.S100B.SPP1, 1; p019, CD.Glial.S100B.SPP1, 0; p022, CD.Glial.S100B.SPP1, 0; p024, CD.Glial.S100B.SPP1, 1; p026,
CD.Glial.S100B.SPP1, 1; p027, CD.Glial.S100B.SPP1, 3; p031, CD.Glial.S100B.SPP1, 3; p032, CD.Glial.S100B.SPP1, 0; p041, CD.Glial.S100B.SPP1, 17; p042,
CD.Glial.S100B.SPP1, 13; p048, CD.Glial.S100B.SPP1, 16; p011, CD.Glial.S100B.SPP1.1, 2; p014, CD.Glial.S100B.SPP1.1, 15; p016, CD.Glial.S100B.SPP1.1, 1; p018,
CD.Glial.S100B.SPP1.1, 0; p019, CD.Glial.S100B.SPP1.1, 0; p022, CD.Glial.S100B.SPP1.1, 1; p024, CD.Glial.S100B.SPP1.1, 1; p026, CD.Glial.S100B.SPP1.1, 1; p027,
CD.Glial.S100B.SPP1.1, 3; p031, CD.Glial.S100B.SPP1.1, 3; p032, CD.Glial.S100B.SPP1.1, 0; p041, CD.Glial.S100B.SPP1.1, 5; p042, CD.Glial.S100B.SPP1.1, 1; p048,
CD.Glial.S100B.SPP1.1, 13; p011, CD.Glial.SPP1.NOV, 0; p014, CD.Glial.SPP1.NOV, 0; p016, CD.Glial.SPP1.NOV, 0; p018, CD.Glial.SPP1.NOV, 0; p019, CD.Glial.SPP1.NOV, 0;
p022, CD.Glial.SPP1.NOV, 1; p024, CD.Glial.SPP1.NOV, 1; p026, CD.Glial.SPP1.NOV, 17; p027, CD.Glial.SPP1.NOV, 2; p031, CD.Glial.SPP1.NOV, 0; p032, CD.Glial.SPP1.NOV, 0;
p041, CD.Glial.SPP1.NOV, 30; p042, CD.Glial.SPP1.NOV, 0; p048, CD.Glial.SPP1.NOV, 0; p011, CD.Glial.SPP1.NRXN1, 2; p014, CD.Glial.SPP1.NRXN1, 3; p016,
CD.Glial.SPP1.NRXN1, 8; p018, CD.Glial.SPP1.NRXN1, 6; p019, CD.Glial.SPP1.NRXN1, 0; p022, CD.Glial.SPP1.NRXN1, 4; p024, CD.Glial.SPP1.NRXN1, 2; p026,
CD.Glial.SPP1.NRXN1, 1; p027, CD.Glial.SPP1.NRXN1, 1; p031, CD.Glial.SPP1.NRXN1, 1; p032, CD.Glial.SPP1.NRXN1, 0; p041, CD.Glial.SPP1.NRXN1, 4; p042,
CD.Glial.SPP1.NRXN1, 20; p048, CD.Glial.SPP1.NRXN1, 5; p011, CD.Glial.XKR4.S100B, 2; p014, CD.Glial.XKR4.S100B, 0; p016, CD.Glial.XKR4.S100B, 0; p018,
CD.Glial.XKR4.S100B, 0; p019, CD.Glial.XKR4.S100B, 0; p022, CD.Glial.XKR4.S100B, 1; p024, CD.Glial.XKR4.S100B, 3; p026, CD.Glial.XKR4.S100B, 0; p027,
CD.Glial.XKR4.S100B, 2; p031, CD.Glial.XKR4.S100B, 34; p032, CD.Glial.XKR4.S100B, 0; p041, CD.Glial.XKR4.S100B, 1; p042, CD.Glial.XKR4.S100B, 1; p048,
CD.Glial.XKR4.S100B, 0; p011, CD.Goblet.BPIFB1.AQP5, 0; p014, CD.Goblet.BPIFB1.AQP5, 0; p016, CD.Goblet.BPIFB1.AQP5, 0; p018, CD.Goblet.BPIFB1.AQP5, 3; p019,
CD.Goblet.BPIFB1.AQP5, 0; p022, CD.Goblet.BPIFB1.AQP5, 82; p024, CD.Goblet.BPIFB1.AQP5, 0; p026, CD.Goblet.BPIFB1.AQP5, 0; p027, CD.Goblet.BPIFB1.AQP5, 0; p031,
CD.Goblet.BPIFB1.AQP5, 0; p032, CD.Goblet.BPIFB1.AQP5, 0; p041, CD.Goblet.BPIFB1.AQP5, 0; p042, CD.Goblet.BPIFB1.AQP5, 0; p048, CD.Goblet.BPIFB1.AQP5, 0; p011,
CD.Goblet.CENPA.UBE2C, 1; p014, CD.Goblet.CENPA.UBE2C, 5; p016, CD.Goblet.CENPA.UBE2C, 3; p018, CD.Goblet.CENPA.UBE2C, 8; p019, CD.Goblet.CENPA.UBE2C, 0;
p022, CD.Goblet.CENPA.UBE2C, 1; p024, CD.Goblet.CENPA.UBE2C, 7; p026, CD.Goblet.CENPA.UBE2C, 7; p027, CD.Goblet.CENPA.UBE2C, 2; p031, CD.Goblet.CENPA.UBE2C,
39; p032, CD.Goblet.CENPA.UBE2C, 1; p041, CD.Goblet.CENPA.UBE2C, 1; p042, CD.Goblet.CENPA.UBE2C, 0; p048, CD.Goblet.CENPA.UBE2C, 6; p011,
CD.Goblet.FCGBP.CLCA1, 1; p014, CD.Goblet.FCGBP.CLCA1, 156; p016, CD.Goblet.FCGBP.CLCA1, 0; p018, CD.Goblet.FCGBP.CLCA1, 0; p019, CD.Goblet.FCGBP.CLCA1, 0;
p022, CD.Goblet.FCGBP.CLCA1, 0; p024, CD.Goblet.FCGBP.CLCA1, 0; p026, CD.Goblet.FCGBP.CLCA1, 0; p027, CD.Goblet.FCGBP.CLCA1, 0; p031, CD.Goblet.FCGBP.CLCA1, 0;
p032, CD.Goblet.FCGBP.CLCA1, 0; p041, CD.Goblet.FCGBP.CLCA1, 0; p042, CD.Goblet.FCGBP.CLCA1, 0; p048, CD.Goblet.FCGBP.CLCA1, 2; p011, CD.Goblet.FCGBP.HES6, 1;
p014, CD.Goblet.FCGBP.HES6, 0; p016, CD.Goblet. FCGBP.HES6, 4; p018, CD.Goblet.FCGBP.HES6, 1; p019, CD.Goblet. FCGBP.HES6, 0; p022, CD.Goblet.FCGBP.HES6, 0; p024,
CD.Goblet.FCGBP.HES6, 0; p026, CD.Goblet.FCGBP.HES6, 4; p027, CD.Goblet.FCGBP.HES6, 3; p031, CD.Goblet.FCGBP.HES6, 2; p032, CD.Goblet.FCGBP.HES6, 0; p041,
CD.Goblet.FCGBP.HES6, 0; p042, CD.Goblet.FCGBP.HES6, 0; p048, CD.Goblet.FCGBP.HES6, 31; p011, CD.Goblet.FCGBP.ITLN1, 3; p014, CD.Goblet.FCGBP.ITLN1, 0; p016,
CD.Goblet.FCGBP.ITLN1, 14; p018, CD.Goblet.FCGBP.ITLN1, 4; p019, CD.Goblet.FCGBP.ITLN1, 0; p022, CD.Goblet.FCGBP.ITLN1, 0; p024, CD.Goblet.FCGBP.ITLN1, 6; p026,
CD.Goblet.FCGBP.ITLN1, 6; p027, CD.Goblet.FCGBP.ITLN1, 10; p031, CD.Goblet.FCGBP.ITLN1, 12; p032, CD.Goblet.FCGBP.ITLN1, 1; p041, CD.Goblet.FCGBP.ITLN1, 1; p042,
CD.Goblet.FCGBP.ITLN1, 1; p048, CD.Goblet.FCGBP.ITLN1, 64; p011, CD.Goblet.FCGBP.SPINK4, 60; p014, CD.Goblet.FCGBP.SPINK4, 1; p016, CD.Goblet.FCGBP.SPINK4, 50;
p018, CD.Goblet.FCGBP.SPINK4, 16; p019, CD.Goblet.FCGBP.SPINK4, 1; p022, CD.Goblet.FCGBP.SPINK4, 19; p024, CD.Goblet.FCGBP.SPINK4, 9; p026,
CD.Goblet.FCGBP.SPINK4, 4; p027, CD.Goblet.FCGBP.SPINK4, 19; p031, CD.Goblet.FCGBP.SPINK4, 33; p032, CD.Goblet.FCGBP.SPINK4, 17; p041, CD.Goblet.FCGBP.SPINK4,
5; p042, CD.Goblet.FCGBP.SPINK4, 2; p048, CD.Goblet.FCGBP.SPINK4, 42; p011, CD.Goblet.HES6.COLCA2, 0; p014, CD.Goblet.HES6.COLCA2, 0; p016,
CD.Goblet.HES6.COLCA2, 0; p018, CD.Goblet.HES6.COLCA2, 9; p019, CD.Goblet.HES6.COLCA2, 0; p022, CD.Goblet.HES6.COLCA2, 2; p024, CD.Goblet.HES6.COLCA2, 4; p026,
CD.Goblet.HES6.COLCA2, 5; p027, CD.Goblet.HES6.COLCA2, 2; p031, CD.Goblet.HES6.COLCA2, 14; p032, CD.Goblet.HES6.COLCA2, 3; p041, CD.Goblet.HES6.COLCA2, 0;
p042, CD.Goblet.HES6.COLCA2, 0; p048, CD.Goblet.HES6.COLCA2, 3; p011, CD.Goblet.ITLN1.CLCA1, 0; p014, CD.Goblet.ITLN1.CLCA1, 1; p016, CD.Goblet.ITLN1.CLCA1, 7;
p018, CD.Goblet.ITLN1.CLCA1, 2; p019, CD.Goblet.ITLN1.CLCA1, 3; p022, CD.Goblet.ITLN1.CLCA1, 0; p024, CD.Goblet.ITLN1.CLCA1, 9; p026, CD.Goblet.ITLN1.CLCA1, 27;
p027, CD.Goblet.ITLN1.CLCA1, 29; p031, CD.Goblet.ITLN1.CLCA1, 106; p032, CD.Goblet.ITLN1.CLCA1, 27; p041, CD.Goblet.ITLN1.CLCA1, 0; p042, CD.Goblet.ITLN1.CLCA1,
1; p048, CD.Goblet.ITLN1.CLCA1, 10; p011, CD.Goblet.ITLN1.CLCA1.1, 0; p014, CD.Goblet.ITLN1.CLCA1.1, 0; p016, CD.Goblet.ITLN1.CLCA1.1, 2; p018,
CD.Goblet.ITLN1.CLCA1.1, 0; p019, CD.Goblet.ITLN1.CLCA1.1, 0; p022, CD.Goblet.ITLN1.CLCA1.1, 1; p024, CD.Goblet.ITLN1.CLCA1.1, 7; p026, CD.Goblet.ITLN1.CLCA1.1,
14; p027, CD.Goblet.ITLN1.CLCA1.1, 4; p031, CD.Goblet.ITLN1.CLCA1.1, 29; p032, CD.Goblet.ITLN1.CLCA1.1, 1; p041, CD.Goblet.ITLN1.CLCA1.1, 0; p042,
CD.Goblet.ITLN1.CLCA1.1, 0; p048, CD.Goblet.ITLN1.CLCA1.1, 0; p011, CD.Goblet. REG4.SPINK4, 45; p014, CD.Goblet.REG4.SPINK4, 6; p016, CD.Goblet.REG4.SPINK4, 7;
p018, CD.Goblet.REG4.SPINK4, 4; p019, CD.Goblet.REG4.SPINK4, 0; p022, CD.Goblet.REG4.SPINK4, 6; p024, CD.Goblet. REG4.SPINK4, 20; p026, CD.Goblet.REG4.SPINK4, 5;
p027, CD.Goblet.REG4.SPINK4, 4; p031, CD.Goblet.REG4.SPINK4, 36; p032, CD.Goblet. REG4.SPINK4, 7; p041, CD.Goblet.REG4.SPINK4, 2; p042, CD.Goblet.REG4.SPINK4, 0;
p048, CD.Goblet.REG4.SPINK4, 10; p011, CD.Goblet.RETNLB.ITLN1, 6; p014, CD.Goblet.RETNLB.ITLN1, 0; p016, CD.Goblet.RETNLB.ITLN1, 0; p018,
CD.Goblet.RETNLB.ITLN1, 10; p019, CD.Goblet.RETNLB.ITLN1, 0; p022, CD.Goblet.RETNLB.ITLN1, 32; p024, CD.Goblet.RETNLB.ITLN1, 15; p026,
CD.Goblet.RETNLB.ITLN1, 6; p027, CD.Goblet.RETNLB.ITLN1, 5; p031, CD.Goblet.RETNLB.ITLN1, 34; p032, CD.Goblet. RETNLB.ITLN1, 2; p041, CD.Goblet.RETNLB.ITLN1,
15; p042, CD.Goblet.RETNLB.ITLN1, 2; p048, CD.Goblet.RETNLB.ITLN1, 6; p011, CD.Goblet.S100P.FCGBP, 0; p014, CD.Goblet.S100P.FCGBP, 0; p016,
CD.Goblet.S100P.FCGBP, 1; p018, CD.Goblet.S100P.FCGBP, 71; p019, CD.Goblet.S100P.FCGBP, 0; p022, CD.Goblet.S100P.FCGBP, 6; p024, CD.Goblet.S100P.FCGBP, 5; p026,
CD.Goblet.S100P.FCGBP, 0; p027, CD.Goblet.S100P.FCGBP, 3; p031, CD.Goblet.S100P.FCGBP, 0; p032, CD.Goblet.S100P.FCGBP, 7; p041, CD.Goblet.S100P.FCGBP, 0; p042,
CD.Goblet.S100P.FCGBP, 0; p048, CD.Goblet.S100P.FCGBP, 2; p011, CD.Goblet.S100P.TFF1, 2; p014, CD.Goblet.S100P.TFF1, 0; p016, CD.Goblet.S100P.TFF1, 3; p018,
CD.Goblet.S100P.TFF1, 113; p019, CD.Goblet.S100P.TFF1, 1; p022, CD.Goblet.S100P.TFF1, 1; p024, CD.Goblet.S100P.TFF1, 1; p026, CD.Goblet.S100P.TFF1, 0; p027,
CD.Goblet.S100P.TFF1, 1; p031, CD.Goblet.S100P.TFF1, 0; p032, CD.Goblet.S100P.TFF1, 6; p041, CD.Goblet.S100P.TFF1, 0; p042, CD.Goblet.S100P.TFF1, 0; p048,
CD.Goblet.S100P.TFF1, 0; p011, CD.Goblet.TFF1.TPSG1, 60; p014, CD.Goblet.TFF1.TPSG1, 0; p016, CD.Goblet.TFF1.TPSG1, 0; p018, CD.Goblet.TFF1.TPSG1, 3; p019,
CD.Goblet.TFF1.TPSG1, 0; p022, CD.Goblet.TFF1.TPSG1, 25; p024, CD.Goblet.TFF1.TPSG1, 3; p026, CD.Goblet.TFF1.TPSG1, 0; p027, CD.Goblet.TFF1.TPSG1, 0; p031,
CD.Goblet.TFF1.TPSG1, 0; p032, CD.Goblet.TFF1.TPSG1, 4; p041, CD.Goblet.TFF1.TPSG1, 7; p042, CD.Goblet.TFF1.TPSG1, 0; p048, CD.Goblet.TFF1.TPSG1, 0; p011,
CD.ILC.AREG.AHR, 2; p014, CD.ILC.AREG.AHR, 18; p016, CD.ILC.AREG.AHR, 0; p018, CD.ILC.AREG.AHR, 1; p019, CD.ILC.AREG.AHR, 0; p022, CD.ILC.AREG.AHR, 0; p024,
CD.ILC.AREG.AHR, 0; p026, CD.ILC.AREG.AHR, 0; p027, CD.ILC.AREG.AHR, 0; p031, CD.ILC.AREG.AHR, 110; p032, CD.ILC.AREG.AHR, 1; p041, CD.ILC.AREG.AHR, 0; p042,
CD.ILC.AREG.AHR, 0; p048, CD.ILC.AREG.AHR, 0; p011, CD.ILC.IL22.KIT, 0; p014, CD.ILC.IL22.KIT, 0; p016, CD.ILC.IL22.KIT, 0; p018, CD.ILC.IL22.KIT, 1; p019,
CD.ILC.IL22.KIT, 79; p022, CD.ILC.IL22.KIT, 0; p024, CD.ILC.IL22.KIT, 0; p026, CD.ILC.IL22.KIT, 0; p027, CD.ILC.IL22.KIT, 5; p031, CD.ILC.IL22.KIT, 0; p032, CD.ILC.IL22.KIT, 0; p041, CD.ILC.IL22.KIT,
0; p042, CD.ILC.IL22.KIT, 2; p048, CD.ILC.IL22.KIT, 0; p011, CD.ILC.KRT81.IL22, 0; p014, CD.ILC.KRT81.IL22, 0; p016, CD.ILC.KRT81.IL22, 0;
p018, CD.ILC.KRT81.IL22, 1; p019, CD.ILC.KRT81.IL22, 0; p022, CD.ILC.KRT81.IL22, 0; p024, CD.ILC.KRT81.IL22, 0; p026, CD.ILC.KRT81.IL22, 70; p027,
CD.ILC.KRT81.IL22, 0; p031, CD.ILC.KRT81.IL22, 0; p032, CD.ILC.KRT81.IL22, 1; p041, CD.ILC.KRT81.IL22, 1; p042, CD.ILC.KRT81.IL22, 0; p048, CD.ILC.KRT81.IL22, 1;
p011, CD.ILC.LST1.AREG, 2; p014, CD.ILC.LST1.AREG, 2; p016, CD.ILC.LST1.AREG, 0; p018, CD.ILC.LST1.AREG, 14; p019, CD.ILC.LST1.AREG, 7; p022, CD.ILC.LST1.AREG, 7;
p024, CD.ILC.LST1.AREG, 6; p026, CD.ILC.LST1.AREG, 2; p027, CD.ILC.LST1.AREG, 7; p031, CD.ILC.LST1.AREG, 4; p032, CD.ILC.LST1.AREG, 9; p041, CD.ILC.LST1.AREG, 6;
p042, CD.ILC.LST1.AREG, 115; p048, CD.ILC.LST1.AREG, 32; p011, CD.Mac.AIF1.HBEGF, 0; p014, CD.Mac.AIF1.HBEGF, 0; p016, CD.Mac.AIF1.HBEGF, 0; p018,
CD.Mac.AIF1.HBEGF, 0; p019, CD.Mac.AIF1.HBEGF, 0; p022, CD.Mac.AIF1.HBEGF, 1; p024, CD.Mac.AIF1.HBEGF, 106; p026, CD.Mac.AIF1.HBEGF, 0; p027,
CD.Mac.AIF1.HBEGF, 0; p031, CD.Mac.AIF1.HBEGF, 4; p032, CD.Mac.AIF1.HBEGF, 0; p041, CD.Mac.AIF1.HBEGF, 1; p042, CD.Mac.AIF1.HBEGF, 0; p048, CD.Mac.AIF1.HBEGF,
0; p011, CD.Mac.APOE.PTGDS, 67; p014, CD.Mac.APOE.PTGDS, 45; p016, CD.Mac.APOE.PTGDS, 31; p018, CD.Mac.APOE.PTGDS, 354; p019, CD.Mac.APOE.PTGDS, 156; p022,
CD.Mac.APOE.PTGDS, 174; p024, CD.Mac.APOE.PTGDS, 511; p026, CD.Mac.APOE.PTGDS, 190; p027, CD.Mac.APOE.PTGDS, 118; p031, CD.Mac.APOE.PTGDS, 309; p032,
CD.Mac.APOE.PTGDS, 134; p041, CD.Mac.APOE.PTGDS, 170; p042, CD.Mac.APOE.PTGDS, 298; p048, CD.Mac.APOE.PTGDS, 309; p011, CD.Mac.C1QB.AIF1, 1; p014,
CD.Mac.C1QB.AIF1, 0; p016, CD.Mac.C1QB.AIF1, 0; p018, CD.Mac.C1QB.AIF1, 0; p019, CD.Mac.C1QB.AIF1, 0; p022, CD.Mac.C1QB.AIF1, 0; p024, CD.Mac.C1QB.AIF1, 71;
p026, CD.Mac.C1QB.AIF1, 0; p027, CD.Mac.C1QB.AIF1, 0; p031, CD.Mac.C1QB.AIF1, 6; p032, CD.Mac.C1QB.AIF1, 0; p041, CD.Mac.C1QB.AIF1, 0; p042, CD.Mac.C1QB.AIF1,
0; p048, CD.Mac.C1QB.AIF1, 0; p011, CD.Mac.C1QB.CD14, 0; p014, CD.Mac.C1QB.CD14, 0; p016, CD.Mac.C1QB.CD14, 0; p018, CD.Mac.C1QB.CD14, 0; p019,
CD.Mac.C1QB.CD14, 0; p022, CD.Mac.C1QB.CD14, 20; p024, CD.Mac.C1QB.CD14, 13; p026, CD.Mac.C1QB.CD14, 0; p027, CD.Mac.C1QB.CD14, 0; p031, CD.Mac.C1QB.CD14,
18; p032, CD.Mac.C1QB.CD14, 1; p041, CD.Mac.C1QB.CD14, 3; p042, CD.Mac.C1QB.CD14, 0; p048, CD.Mac.C1QB.CD14, 2; p011, CD.Mac.C1QB.FCGR1A, 3; p014,
CD.Mac.C1QB.FCGR1A, 0; p016, CD.Mac.C1QB.FCGR1A, 1; p018, CD.Mac.C1QB.FCGR1A, 32; p019, CD.Mac.C1QB.FCGR1A, 1; p022, CD.Mac.C1QB.FCGR1A, 0; p024,
CD.Mac.C1QB.FCGR1A, 0; p026, CD.Mac.C1QB.FCGR1A, 0; p027, CD.Mac.C1QB.FCGR1A, 1; p031, CD.Mac.C1QB.FCGR1A, 0; p032, CD.Mac.C1QB.FCGR1A, 34; p041,
CD.Mac.C1QB.FCGR1A, 27; p042, CD.Mac.C1QB.FCGR1A, 3; p048, CD.Mac.C1QB.FCGR1A, 1; p011, CD.Mac.CXCL2.CXCL3, 4; p014, CD.Mac.CXCL2.CXCL3, 1; p016,
CD.Mac.CXCL2.CXCL3, 0; p018, CD.Mac.CXCL2.CXCL3, 0; p019, CD.Mac.CXCL2.CXCL3, 2; p022, CD.Mac.CXCL2.CXCL3, 8; p024, CD.Mac.CXCL2.CXCL3, 1; p026,
CD.Mac.CXCL2.CXCL3, 1; p027, CD.Mac.CXCL2.CXCL3, 0; p031, CD.Mac.CXCL2.CXCL3, 4; p032, CD.Mac.CXCL2.CXCL3, 1; p041, CD.Mac.CXCL2.CXCL3, 47; p042,
CD.Mac.CXCL2.CXCL3, 11; p048, CD.Mac.CXCL2.CXCL3, 5; p011, CD.Mac.CXCL2.TNF, 0; p014, CD.Mac.CXCL2.TNF, 0; p016, CD.Mac.CXCL2.TNF, 0; p018, CD.Mac.CXCL2.TNF,
0; p019, CD.Mac.CXCL2.TNF, 0; p022, CD.Mac.CXCL2.TNF, 0; p024, CD.Mac.CXCL2.TNF, 0; p026, CD.Mac.CXCL2.TNF, 0; p027, CD.Mac.CXCL2.TNF, 0; p031,
CD.Mac.CXCL2.TNF, 0; p032, CD.Mac.CXCL2.TNF, 0; p041, CD.Mac.CXCL2.TNF, 49; p042, CD.Mac.CXCL2.TNF, 0; p048, CD.Mac.CXCL2.TNF, 1; p011, CD.Mac.CXCL3.APOC1,
1; p014, CD.Mac.CXCL3.APOC1, 1; p016, CD.Mac.CXCL3.APOC1, 0; p018, CD.Mac.CXCL3.APOC1, 3; p019, CD.Mac.CXCL3.APOC1, 0; p022, CD.Mac.CXCL3.APOC1, 23; p024,
CD.Mac.CXCL3.APOC1, 7; p026, CD.Mac.CXCL3.APOC1, 0; p027, CD.Mac.CXCL3.APOC1, 0; p031, CD.Mac.CXCL3.APOC1, 16; p032, CD.Mac.CXCL3.APOC1, 0; p041,
CD.Mac.CXCL3.APOC1, 6; p042, CD.Mac.CXCL3.APOC1, 0; p048, CD.Mac.CXCL3.APOC1, 0; p011, CD.Mac.CXCL8.HES1, 0; p014, CD.Mac.CXCL8.HES1, 1; p016,
CD.Mac.CXCL8.HES1, 0; p018, CD.Mac.CXCL8.HES1, 1; p019, CD.Mac.CXCL8.HES1, 0; p022, CD.Mac.CXCL8.HES1, 0; p024, CD.Mac.CXCL8.HES1, 0; p026, CD.Mac.CXCL8.HES1,
0; p027, CD.Mac.CXCL8.HES1, 1; p031, CD.Mac.CXCL8.HES1, 1; p032, CD.Mac.CXCL8.HES1, 0; p041, CD.Mac.CXCL8.HES1, 1; p042, CD.Mac.CXCL8.HES1, 2; p048,
CD.Mac.CXCL8.HES1, 51; p011, CD.Mac.FOLR2.LILRB5, 0; p014, CD.Mac.FOLR2.LILRB5, 3; p016, CD.Mac.FOLR2.LILRB5, 0; p018, CD.Mac.FOLR2.LILRB5, 0; p019,
CD.Mac. FOLR2.LILRB5, 5; p022, CD.Mac.FOLR2.LILRB5, 0; p024, CD.Mac.FOLR2.LILRB5, 1; p026, CD.Mac.FOLR2.LILRB5, 5; p027, CD.Mac.FOLR2.LILRB5, 1; p031,
CD.Mac. FOLR2.LILRB5, 3; p032, CD.Mac.FOLR2.LILRB5, 0; p041, CD.Mac.FOLR2.LILRB5, 3; p042, CD.Mac.FOLR2.LILRB5, 282; p048, CD.Mac.FOLR2.LILRB5, 14; p011,
CD.Mac.IGSF6.TXNIP, 1; p014, CD.Mac.IGSF6.TXNIP, 0; p016, CD.Mac.IGSF6.TXNIP, 0; p018, CD.Mac.IGSF6.TXNIP, 1; p019, CD.Mac.IGSF6.TXNIP, 0; p022,
CD.Mac.IGSF6.TXNIP, 1; p024, CD.Mac.IGSF6.TXNIP, 62; p026, CD.Mac.IGSF6.TXNIP, 0; p027, CD.Mac.IGSF6.TXNIP, 1; p031, CD.Mac.IGSF6.TXNIP, 9; p032,
CD.Mac.IGSF6.TXNIP, 0; p041, CD.Mac.IGSF6.TXNIP, 0; p042, CD.Mac.IGSF6.TXNIP, 0; p048, CD.Mac.IGSF6.TXNIP, 2; p011, CD.Mac.JUN.EGR1, 49; p014, CD.Mac.JUN.EGR1,
2; p016, CD.Mac.JUN.EGR1, 5; p018, CD.Mac.JUN.EGR1, 2; p019, CD.Mac.JUN.EGR1, 0; p022, CD.Mac.JUN.EGR1, 0; p024, CD.Mac.JUN.EGR1, 0; p026, CD.Mac.JUN.EGR1, 0;
p027, CD.Mac.JUN.EGR1, 1; p031, CD.Mac.JUN.EGR1, 0; p032, CD.Mac.JUN.EGR1, 0; p041, CD.Mac.JUN.EGR1, 1; p042, CD.Mac.JUN.EGR1, 0; p048, CD.Mac.JUN.EGR1, 3; p011,
CD.Mac.LYZ.TXN, 0; p014, CD.Mac.LYZ.TXN, 0; p016, CD.Mac.LYZ.TXN, 0; p018, CD.Mac.LYZ.TXN, 0; p019, CD.Mac.LYZ.TXN, 0; p022, CD.Mac.LYZ.TXN, 0; p024,
CD.Mac.LYZ.TXN, 77; p026, CD.Mac.LYZ.TXN, 0; p027, CD.Mac.LYZ.TXN, 1; p031, CD.Mac.LYZ.TXN, 1; p032, CD.Mac.LYZ.TXN, 0; p041, CD.Mac.LYZ.TXN, 0; p042,
CD.Mac.LYZ.TXN, 0; p048, CD.Mac.LYZ.TXN, 0; p011, CD.Mac.MKI67.APOE, 0; p014, CD.Mac.MKI67.APOE, 3; p016, CD.Mac.MKI67.APOE, 2; p018, CD.Mac.MKI67.APOE, 11;
p019, CD.Mac.MKI67.APOE, 1; p022, CD.Mac.MKI67.APOE, 1; p024, CD.Mac.MKI67.APOE, 17; p026, CD.Mac.MKI67.APOE, 1; p027, CD.Mac.MKI67.APOE, 4; p031,
CD.Mac.MKI67.APOE, 8; p032, CD.Mac.MKI67.APOE, 1; p041, CD.Mac.MKI67.APOE, 4; p042, CD.Mac.MKI67.APOE, 9; p048, CD.Mac.MKI67.APOE, 6; p011,
CD.Mac.SEPP1.CXCL3, 3; p014, CD.Mac.SEPP1.CXCL3, 5; p016, CD.Mac.SEPP1.CXCL3, 1; p018, CD.Mac.SEPP1.CXCL3, 0; p019, CD.Mac.SEPP1.CXCL3, 0; p022,
CD.Mac.SEPP1.CXCL3, 2; p024, CD.Mac.SEPP1.CXCL3, 6; p026, CD.Mac.SEPP1.CXCL3, 13; p027, CD.Mac.SEPP1.CXCL3, 2; p031, CD.Mac.SEPP1.CXCL3, 22; p032,
CD.Mac.SEPP1.CXCL3, 0; p041, CD.Mac.SEPP1.CXCL3, 25; p042, CD.Mac.SEPP1.CXCL3, 60; p048, CD.Mac.SEPP1.CXCL3, 19; p011, CD.Mac/cDC1.MKI67.IDO1, 4; p014,
CD.Mac/cDC1.MKI67.IDO1, 1; p016, CD.Mac/cDC1.MKI67.IDO1, 3; p018, CD.Mac/cDC1.MKI67.IDO1, 5; p019, CD.Mac/cDC1.MKI67.IDO1, 4; p022,
CD.Mac/cDC1.MKI67.IDO1, 1; p024, CD.Mac/cDC1.MKI67.IDO1, 9; p026, CD.Mac/cDC1.MKI67.IDO1, 29; p027, CD.Mac/cDC1.MKI67.IDO1, 1; p031,
CD.Mac/cDC1.MKI67.IDO1, 15; p032, CD.Mac/cDC1.MKI67.IDO1, 1; p041, CD.Mac/cDC1.MKI67.IDO1, 7; p042, CD.Mac/cDC1.MKI67.IDO1, 3; p048,
CD.Mac/cDC1.MKI67.IDO1, 2; p011, CD.Mac/DC.CXCL10.CCL19, 1; p014, CD.Mac/DC.CXCL10.CCL19, 0; p016, CD.Mac/DC.CXCL10.CCL19, 0; p018,
CD.Mac/DC.CXCL10.CCL19, 39; p019, CD.Mac/DC.CXCL10.CCL19, 1; p022, CD.Mac/DC.CXCL10.CCL19, 0; p024, CD.Mac/DC.CXCL10.CCL19, 0; p026,
CD.Mac/DC.CXCL10.CCL19, 0; p027, CD.Mac/DC.CXCL10.CCL19, 0; p031, CD.Mac/DC.CXCL10.CCL19, 0; p032, CD.Mac/DC.CXCL10.CCL19, 0; p041,
CD.Mac/DC.CXCL10.CCL19, 0; p042, CD.Mac/DC.CXCL10.CCL19, 0; p048, CD.Mac/DC.CXCL10.CCL19, 0; p011, CD.Mac/DC.CXCL10.CLEC4E, 2; p014,
CD.Mac/DC.CXCL10.CLEC4E, 0; p016, CD.Mac/DC.CXCL10.CLEC4E, 1; p018, CD.Mac/DC.CXCL10.CLEC4E, 2; p019, CD.Mac/DC.CXCL10.CLEC4E, 0; p022,
CD.Mac/DC.CXCL10.CLEC4E, 8; p024, CD.Mac/DC.CXCL10.CLEC4E, 74; p026, CD.Mac/DC.CXCL10.CLEC4E, 0; p027, CD. Mac/DC.CXCL10.CLEC4E, 2; p031,
CD.Mac/DC.CXCL10.CLEC4E, 35; p032, CD.Mac/DC.CXCL10.CLEC4E, 6; p041, CD.Mac/DC.CXCL10.CLEC4E, 6; p042, CD.Mac/DC.CXCL10.CLEC4E, 2; p048,
CD.Mac/DC.CXCL10.CLEC4E, 1; p011, CD.Mac/DC.CXCL8.NLRP3, 0; p014, CD.Mac/DC.CXCL8.NLRP3, 0; p016, CD.Mac/DC.CXCL8.NLRP3, 0; p018,
CD.Mac/DC.CXCL8.NLRP3, 0; p019, CD.Mac/DC.CXCL8.NLRP3, 1; p022, CD.Mac/DC.CXCL8.NLRP3, 0; p024, CD.Mac/DC.CXCL8.NLRP3, 0; p026, CD.Mac/DC.CXCL8.NLRP3,
0; p027, CD.Mac/DC.CXCL8.NLRP3, 0; p031, CD.Mac/DC.CXCL8.NLRP3, 0; p032, CD.Mac/DC.CXCL8.NLRP3, 0; p041, CD.Mac/DC.CXCL8.NLRP3, 13; p042
CD.Mac/DC.CXCL8.NLRP3, 61; p048, CD.Mac/DC.CXCL8.NLRP3, 2; p011, CD.Mcell.CCL23.SPIB, 0; p014, CD.Mcell.CCL23.SPIB, 0; p016, CD.Mcell.CCL23.SPIB, 3; p018,
CD.Mcell.CCL23.SPIB, 12; p019, CD.Mcell.CCL23.SPIB, 10; p022, CD.Mcell.CCL23.SPIB, 0; p024, CD.Mcell.CCL23.SPIB, 0; p026, CD.Mcell.CCL23.SPIB, 124; p027,
CD.Mcell.CCL23.SPIB, 21; p031, CD.Mcell.CCL23.SPIB, 2; p032, CD.Mcell.CCL23.SPIB, 3; p041, CD.Mcell.CCL23.SPIB, 0; p042, CD.Mcell.CCL23.SPIB, 2; p048,
CD.Mcell.CCL23.SPIB, 1; p011, CD.Mcell.CSRP2.SPIB, 0; p014, CD.Mcell.CSRP2.SPIB, 0; p016, CD.Mcell.CSRP2.SPIB, 0; p018, CD.Mcell.CSRP2.SPIB, 0; p019,
CD.Mcell.CSRP2.SPIB, 25; p022, CD.Mcell.CSRP2.SPIB, 0; p024, CD.Mcell.CSRP2.SPIB, 0; p026, CD.Mcell.CSRP2.SPIB, 0; p027, CD.Mcell.CSRP2.SPIB, 1; p031,
CD.Mcell.CSRP2.SPIB, 0; p032, CD.Mcell.CSRP2.SPIB, 0; p041, CD.Mcell.CSRP2.SPIB, 0; p042, CD.Mcell.CSRP2.SPIB, 3; p048, CD.Mcell.CSRP2.SPIB, 9; p011,
CD.Mono.CXCL10.TNF, 6; p014, CD.Mono.CXCL10.TNF, 2; p016, CD.Mono.CXCL10.TNF, 2; p018, CD.Mono.CXCL10.TNF, 0; p019, CD.Mono.CXCL10.TNF, 0; p022,
CD.Mono.CXCL10.TNF, 152; p024, CD.Mono.CXCL10.TNF, 9; p026, CD.Mono.CXCL10.TNF, 3; p027, CD.Mono.CXCL10.TNF, 0; p031, CD.Mono.CXCL10.TNF, 19; p032,
CD.Mono.CXCL10.TNF, 1; p041, CD.Mono.CXCL10.TNF, 124; p042, CD.Mono.CXCL10.TNF, 2; p048, CD.Mono.CXCL10.TNF, 0; p011, CD.Mono.CXCL3.FCN1, 2; p014,
CD.Mono.CXCL3.FCN1, 4; p016, CD.Mono.CXCL3.FCN1, 3; p018, CD.Mono.CXCL3.FCN1, 1; p019, CD.Mono.CXCL3.FCN1, 0; p022, CD.Mono.CXCL3.FCN1, 53; p024,
CD.Mono.CXCL3.FCN1, 44; p026, CD.Mono.CXCL3.FCN1, 0; p027, CD.Mono.CXCL3.FCN1, 1; p031, CD.Mono.CXCL3.FCN1, 24; p032, CD.Mono.CXCL3.FCN1, 3; p041,
CD.Mono.CXCL3.FCN1, 74; p042, CD.Mono.CXCL3.FCN1, 5; p048, CD.Mono.CXCL3.FCN1, 7; p011, CD.Mono.FCN1.AREG, 0; p014, CD.Mono.FCN1.AREG, 1; p016,
CD.Mono.FCN1.AREG, 0; p018, CD.Mono.FCN1.AREG, 0; p019, CD.Mono.FCN1.AREG, 0; p022, CD.Mono.FCN1.AREG, 2; p024, CD.Mono.FCN1.AREG, 4; p026,
CD.Mono.FCN1.AREG, 0; p027, CD.Mono.FCN1.AREG, 0; p031, CD.Mono.FCN1.AREG, 99; p032, CD.Mono.FCN1.AREG, 0; p041, CD.Mono.FCN1.AREG, 0; p042,
CD.Mono.FCN1.AREG, 0; p048, CD.Mono.FCN1.AREG, 0; p011, CD.Mono.FCN1.EIF4A3, 40; p014, CD.Mono.FCN1.EIF4A3, 0; p016, CD.Mono.FCN1.EIF4A3, 7; p018,
CD.Mono.FCN1.EIF4A3, 1; p019, CD.Mono.FCN1.EIF4A3, 0; p022, CD.Mono.FCN1.EIF4A3, 0; p024, CD.Mono.FCN1.EIF4A3, 0; p026, CD.Mono.FCN1.EIF4A3, 0; p027,
CD.Mono.FCN1.EIF4A3, 0; p031, CD.Mono.FCN1.EIF4A3, 0; p032, CD.Mono.FCN1.EIF4A3, 0; p041, CD.Mono.FCN1.EIF4A3, 1; p042, CD.Mono.FCN1.EIF4A3, 0; p048,
CD.Mono.FCN1.EIF4A3, 6; p011, CD.Mono.FCN1.HSPA1A, 96; p014, CD.Mono.FCN1.HSPA1A, 0; p016, CD.Mono.FCN1.HSPA1A, 1; p018, CD.Mono.FCN1.HSPA1A, 0; p019,
CD.Mono.FCN1.HSPA1A, 0; p022, CD.Mono.FCN1.HSPA1A, 0; p024, CD.Mono.FCN1.HSPA1A, 0; p026, CD.Mono.FCN1.HSPA1A, 0; p027, CD.Mono.FCN1.HSPA1A, 0; p031,
CD.Mono.FCN1.HSPA1A, 0; p032, CD.Mono.FCN1.HSPA1A, 0; p041, CD.Mono.FCN1.HSPA1A, 2; p042, CD.Mono.FCN1.HSPA1A, 0; p048, CD.Mono.FCN1.HSPA1A, 1; p011,
CD.Mono.FCN1.LYST, 0; p014, CD.Mono.FCN1.LYST, 0; p016, CD.Mono.FCN1.LYST, 1; p018, CD.Mono.FCN1.LYST, 4; p019, CD.Mono.FCN1.LYST, 0; p022,
CD.Mono.FCN1.LYST, 2; p024, CD.Mono.FCN1.LYST, 8; p026, CD.Mono.FCN1.LYST, 0; p027, CD.Mono.FCN1.LYST, 1; p031, CD.Mono.FCN1.LYST, 7; p032,
CD.Mono.FCN1.LYST, 0; p041, CD.Mono.FCN1.LYST, 0; p042, CD.Mono.FCN1.LYST, 0; p048, CD.Mono.FCN1.LYST, 3; p011, CD.Mono.FCN1.S100A4, 22; p014,
CD.Mono.FCN1.S100A4, 5; p016, CD.Mono.FCN1.S100A4, 2; p018, CD.Mono.FCN1.S100A4, 32; p019, CD.Mono.FCN1.S100A4, 3; p022, CD.Mono.FCN1.S100A4, 40; p024,
CD.Mono.FCN1.S100A4, 374; p026, CD.Mono.FCN1.S100A4, 6; p027, CD.Mono.FCN1.S100A4, 6; p031, CD.Mono.FCN1.S100A4, 46; p032, CD.Mono.FCN1.S100A4, 76; p041,
CD.Mono.FCN1.S100A4, 59; p042, CD.Mono.FCN1.S100A4, 6; p048, CD.Mono.FCN1.S100A4, 7; p011, CD.Mono.S100A8.S100A9, 44; p014, CD.Mono.S100A8.S100A9, 8;
p016, CD.Mono.S100A8.S100A9, 1; p018, CD.Mono.S100A8.S100A9, 7; p019, CD.Mono.S100A8.S100A9, 0; p022, CD.Mono.S100A8.S100A9, 47; p024,
CD.Mono.S100A8.S100A9, 36; p026, CD.Mono.S100A8.S100A9, 41; p027, CD.Mono.S100A8.S100A9, 1; p031, CD.Mono.S100A8.S100A9, 58; p032,
CD.Mono.S100A8.S100A9, 14; p041, CD.Mono.S100A8.S100A9, 369; p042, CD.Mono.S100A8.S100A9, 5; p048, CD.Mono.S100A8.S100A9, 2; p011,
CD.Mono/cDC2.CLEC10A.AREG, 0; p014, CD.Mono/cDC2.CLEC10A.AREG, 0; p016, CD.Mono/cDC2.CLEC10A.AREG, 0; p018, CD.Mono/cDC2.CLEC10A.AREG, 3; p019,
CD.Mono/cDC2.CLEC10A.AREG, 4; p022, CD.Mono/cDC2.CLEC10A.AREG, 0; p024, CD.Mono/cDC2.CLEC10A.AREG, 0; p026, CD.Mono/cDC2.CLEC10A.AREG, 0; p027,
CD.Mono/cDC2.CLEC10A.AREG, 6; p031, CD.Mono/cDC2.CLEC10A.AREG, 0; p032, CD.Mono/cDC2.CLEC10A.AREG, 0; p041, CD.Mono/cDC2.CLEC10A.AREG, 3; p042,
CD.Mono/cDC2.CLEC10A.AREG, 66; p048, CD.Mono/cDC2.CLEC10A.AREG, 0; p011, CD.Mono/cDC2.CLEC10A.SDS, 0; p014, CD.Mono/cDC2.CLEC10A.SDS, 0; p016,
CD.Mono/cDC2.CLEC10A.SDS, 0; p018, CD.Mono/cDC2.CLEC10A.SDS, 0; p019, CD.Mono/cDC2.CLEC10A.SDS, 0; p022, CD.Mono/cDC2.CLEC10A.SDS, 0; p024,
CD.Mono/cDC2.CLEC10A.SDS, 0; p026, CD.Mono/cDC2.CLEC10A.SDS, 0; p027, CD.Mono/cDC2.CLEC10A.SDS, 0; p031, CD.Mono/cDC2.CLEC10A.SDS, 0; p032,
CD.Mono/cDC2.CLEC10A.SDS, 0; p041, CD.Mono/cDC2.CLEC10A.SDS, 0; p042, CD.Mono/cDC2.CLEC10A.SDS, 0; p048, CD.Mono/cDC2.CLEC10A.SDS, 68; p011,
CD.Mono/cDC2.FCN1.CD1C, 0; p014, CD.Mono/cDC2.FCN1.CD1C, 2; p016, CD.Mono/cDC2.FCN1.CD1C, 3; p018, CD.Mono/cDC2.FCN1.CD1C, 2; p019,
CD.Mono/cDC2.FCN1.CD1C, 2; p022, CD.Mono/cDC2.FCN1.CD1C, 6; p024, CD.Mono/cDC2.FCN1.CD1C, 3; p026, CD.Mono/cDC2.FCN1.CD1C, 1; p027,
CD.Mono/cDC2.FCN1.CD1C, 4; p031, CD.Mono/cDC2.FCN1.CD1C, 3; p032, CD.Mono/cDC2.FCN1.CD1C, 2; p041, CD.Mono/cDC2.FCN1.CD1C, 7; p042,
CD.Mono/cDC2.FCN1.CD1C, 4; p048, CD.Mono/cDC2.FCN1.CD1C, 1; p011, CD.Mono/Mac.CCL4.DDX3Y, 62; p014, CD.Mono/Mac.CCL4.DDX3Y, 2; p016,
CD.Mono/Mac.CCL4.DDX3Y, 0; p018, CD.Mono/Mac.CCL4.DDX3Y, 0; p019, CD.Mono/Mac.CCL4.DDX3Y, 0; p022, CD.Mono/Mac.CCL4.DDX3Y, 0; p024,
CD.Mono/Mac.CCL4.DDX3Y, 0; p026, CD.Mono/Mac.CCL4.DDX3Y, 0; p027, CD.Mono/Mac.CCL4.DDX3Y, 0; p031, CD.Mono/Mac.CCL4.DDX3Y, 0; p032,
CD.Mono/Mac.CCL4.DDX3Y, 0; p041, CD.Mono/Mac.CCL4.DDX3Y, 0; p042, CD.Mono/Mac.CCL4.DDX3Y, 0; p048, CD.Mono/Mac.CCL4.DDX3Y, 0; p011,
CD.Mono/Mac.CXCL10.CXCL11, 4; p014, CD.Mono/Mac.CXCL10.CXCL11, 2; p016, CD.Mono/Mac.CXCL10.CXCL11, 0; p018, CD.Mono/Mac.CXCL10.CXCL11, 0; p019,
CD.Mono/Mac.CXCL10.CXCL11, 2; p022, CD.Mono/Mac.CXCL10.CXCL11, 34; p024, CD.Mono/Mac.CXCL10.CXCL11, 9; p026, CD.Mono/Mac.CXCL10.CXCL11, 1; p027,
CD.Mono/Mac.CXCL10.CXCL11, 0; p031, CD.Mono/Mac.CXCL10.CXCL11, 3; p032, CD.Mono/Mac.CXCL10.CXCL11, 5; p041, CD.Mono/Mac.CXCL10.CXCL11, 43; p042,
CD.Mono/Mac.CXCL10.CXCL11, 1; p048, CD.Mono/Mac.CXCL10.CXCL11, 0; p011, CD.Mono/Mac.CXCL10.FCN1, 1; p014, CD.Mono/Mac.CXCL10.FCN1, 1; p016,
CD.Mono/Mac.CXCL10.FCN1, 0; p018, CD.Mono/Mac.CXCL10.FCN1, 5; p019, CD.Mono/Mac.CXCL10.FCN1, 0; p022, CD.Mono/Mac.CXCL10.FCN1, 22; p024,
CD.Mono/Mac.CXCL10.FCN1, 26; p026, CD.Mono/Mac.CXCL10.FCN1, 0; p027, CD.Mono/Mac.CXCL10.FCN1, 1; p031, CD.Mono/Mac.CXCL10.FCN1, 12; p032,
CD.Mono/Mac.CXCL10.FCN1, 18; p041, CD.Mono/Mac.CXCL10.FCN1, 62; p042, CD.Mono/Mac.CXCL10.FCN1, 0; p048, CD.Mono/Mac.CXCL10.FCN1, 0; p011,
CD.Mono/Mac.CXCL10.GBP1, 8; p014, CD.Mono/Mac.CXCL10.GBP1, 3; p016, CD.Mono/Mac.CXCL10.GBP1, 0; p018, CD.Mono/Mac.CXCL10.GBP1, 6; p019,
CD.Mono/Mac.CXCL10.GBP1, 2; p022, CD.Mono/Mac.CXCL10.GBP1, 16; p024, CD.Mono/Mac.CXCL10.GBP1, 13; p026, CD.Mono/Mac.CXCL10.GBP1, 0; p027,
CD.Mono/Mac.CXCL10.GBP1, 8; p031, CD.Mono/Mac.CXCL10.GBP1, 12; p032, CD.Mono/Mac.CXCL10.GBP1, 45; p041, CD.Mono/Mac.CXCL10.GBP1, 15; p042,
CD.Mono/Mac.CXCL10.GBP1, 6; p048, CD.Mono/Mac.CXCL10.GBP1, 3; p011, CD.Mono/Mac.CXCL10.LYZ, 0; p014, CD.Mono/Mac.CXCL10.LYZ, 0; p016,
CD.Mono/Mac.CXCL10.LYZ, 0; p018, CD.Mono/Mac.CXCL10.LYZ, 0; p019, CD.Mono/Mac.CXCL10.LYZ, 0; p022, CD.Mono/Mac.CXCL10.LYZ, 2; p024,
CD.Mono/Mac.CXCL10.LYZ, 0; p026, CD.Mono/Mac.CXCL10.LYZ, 0; p027, CD.Mono/Mac.CXCL10.LYZ, 0; p031, CD.Mono/Mac.CXCL10.LYZ, 2; p032,
CD.Mono/Mac.CXCL10.LYZ, 4; p041, CD.Mono/Mac.CXCL10.LYZ, 19; p042, CD.Mono/Mac.CXCL10.LYZ, 0; p048, CD.Mono/Mac.CXCL10.LYZ, 0; p011,
CD.Mono/Mac/DC.CXCL10.IFIT2, 36; p014, CD.Mono/Mac/DC.CXCL10.IFIT2, 0; p016, CD.Mono/Mac/DC.CXCL10.IFIT2, 1; p018, CD.Mono/Mac/DC.CXCL10.IFIT2, 0; p019,
CD.Mono/Mac/DC.CXCL10.IFIT2, 0; p022, CD.Mono/Mac/DC.CXCL10.IFIT2, 0; p024, CD.Mono/Mac/DC.CXCL10.IFIT2, 0; p026, CD.Mono/Mac/DC.CXCL10.IFIT2, 0; p027,
CD.Mono/Mac/DC.CXCL10.IFIT2, 0; p031, CD.Mono/Mac/DC.CXCL10.IFIT2, 0; p032, CD.Mono/Mac/DC.CXCL10.IFIT2, 0; p041, CD.Mono/Mac/DC.CXCL10.IFIT2, 1; p042,
CD.Mono/Mac/DC.CXCL10.IFIT2, 0; p048, CD.Mono/Mac/DC.CXCL10.IFIT2, 0; p011, CD.Mono/Mac/DC.CXCL8.CLEC10A, 1; p014, CD.Mono/Mac/DC.CXCL8.CLEC10A, 0;
p016, CD.Mono/Mac/DC.CXCL8.CLEC10A, 0; p018, CD.Mono/Mac/DC.CXCL8.CLEC10A, 0; p019, CD.Mono/Mac/DC.CXCL8.CLEC10A, 1; p022,
CD.Mono/Mac/DC.CXCL8.CLEC10A, 1; p024, CD.Mono/Mac/DC.CXCL8.CLEC10A, 1; p026, CD.Mono/Mac/DC.CXCL8.CLEC10A, 0; p027, CD.Mono/Mac/DC.CXCL8.CLEC10A,
1; p031, CD.Mono/Mac/DC.CXCL8.CLEC10A, 1; p032, CD.Mono/Mac/DC.CXCL8.CLEC10A, 1; p041, CD.Mono/Mac/DC.CXCL8.CLEC10A, 0; p042,
CD.Mono/Mac/DC.CXCL8.CLEC10A, 2; p048, CD.Mono/Mac/DC.CXCL8.CLEC10A, 12; p011, CD.Mstcl.AREG.ADCYAP1, 1; p014, CD.Mstcl.AREG.ADCYAP1, 0; p016,
CD.Mstcl.AREG.ADCYAP1, 0; p018, CD.Mstcl.AREG.ADCYAP1, 2; p019, CD.Mstcl.AREG.ADCYAP1, 0; p022, CD.Mstcl.AREG.ADCYAP1, 44; p024, CD.Mstcl.AREG.ADCYAP1, 4;
p026, CD.Mstcl.AREG.ADCYAP1, 2; p027, CD.Mstcl.AREG.ADCYAP1, 2; p031, CD.Mstcl.AREG.ADCYAP1, 5; p032, CD.Mstcl.AREG.ADCYAP1, 29; p041,
CD.Mstcl.AREG.ADCYAP1, 96; p042, CD.Mstcl.AREG.ADCYAP1, 4; p048, CD.Mstcl.AREG.ADCYAP1, 1; p011, CD.Mstcl.CD3D.CD2, 25; p014, CD.Mstcl.CD3D.CD2, 2; p016,
CD.Mstcl.CD3D.CD2, 2; p018, CD.Mstcl.CD3D.CD2, 32; p019, CD.Mstcl.CD3D.CD2, 1; p022, CD.Mstcl.CD3D.CD2, 1; p024, CD.Mstcl.CD3D.CD2, 1; p026, CD.Mstcl.CD3D.CD2,
1; p027, CD.Mstcl.CD3D.CD2, 3; p031, CD.Mstcl.CD3D.CD2, 2; p032, CD.Mstcl.CD3D.CD2, 3; p041, CD.Mstcl.CD3D.CD2, 2; p042, CD.Mstcl.CD3D.CD2, 2; p048,
CD.Mstcl.CD3D.CD2, 2; p011, CD.Mstcl.CDK1.KIAA0101, 8; p014, CD.Mstcl.CDK1.KIAA0101, 2; p016, CD.Mstcl.CDK1.KIAA0101, 0; p018, CD.Mstcl.CDK1.KIAA0101, 0; p019,
CD.Mstcl.CDK1.KIAA0101, 0; p022, CD.Mstcl.CDK1.KIAA0101, 0; p024, CD.Mstcl.CDK1.KIAA0101, 6; p026, CD.Mstcl.CDK1.KIAA0101, 0; p027, CD.Mstcl.CDK1.KIAA0101,
2; p031, CD.Mstcl.CDK1.KIAA0101, 4; p032, CD.Mstcl.CDK1.KIAA0101, 1; p041, CD.Mstcl.CDK1.KIAA0101, 3; p042, CD.Mstcl.CDK1.KIAA0101, 6; p048,
CD.Mstcl.CDK1.KIAA0101, 0; p011, CD.Mstcl.CTSG.CD69, 8; p014, CD.Mstcl.CTSG.CD69, 7; p016, CD.Mstcl.CTSG.CD69, 1; p018, CD.Mstcl.CTSG.CD69, 5; p019,
CD.Mstcl.CTSG.CD69, 17; p022, CD.Mstcl.CTSG.CD69, 22; p024, CD.Mstcl.CTSG.CD69, 14; p026, CD.Mstcl.CTSG.CD69, 31; p027, CD.Mstcl.CTSG.CD69, 64; p031,
CD.Mstcl.CTSG.CD69, 144; p032, CD.Mstcl.CTSG.CD69, 29; p041, CD.Mstcl.CTSG.CD69, 70; p042, CD.Mstcl.CTSG.CD69, 264; p048, CD.Mstcl.CTSG.CD69, 19; p011,
CD.Mstcl.DEFA5.RPLP2, 4; p014, CD.Mstcl.DEFA5.RPLP2, 1; p016, CD.Mstcl.DEFA5.RPLP2, 0; p018, CD.Mstcl.DEFA5.RPLP2, 263; p019, CD.Mstcl.DEFA5.RPLP2, 8; p022,
CD.Mstcl.DEFA5.RPLP2, 1; p024, CD.Mstcl.DEFA5.RPLP2, 1; p026, CD.Mstcl.DEFA5.RPLP2, 1; p027, CD.Mstcl.DEFA5.RPLP2, 14; p031, CD.Mstcl.DEFA5.RPLP2, 1; p032,
CD.Mstcl.DEFA5.RPLP2, 6; p041, CD.Mstcl.DEFA5.RPLP2, 8; p042, CD.Mstcl.DEFA5.RPLP2, 5; p048, CD.Mstcl.DEFA5.RPLP2, 13; p011, CD.Mstcl.FOS.JUN, 4; p014,
CD.Mstcl.FOS.JUN, 4; p016, CD.Mstcl.FOS.JUN, 3; p018, CD.Mstcl.FOS.JUN, 1; p019, CD.Mstcl.FOS.JUN, 6; p022, CD.Mstcl.FOS.JUN, 8; p024, CD.Mstcl.FOS.JUN, 10; p026,
CD. Mstcl.FOS.JUN, 14; p027, CD.Mstcl.FOS.JUN, 22; p031, CD.Mstcl.FOS.JUN, 10; p032, CD.Mstcl.FOS.JUN, 7; p041, CD.Mstcl.FOS.JUN, 30; p042, CD.Mstcl.FOS.JUN, 33; p048,
CD.Mstcl.FOS.JUN, 23; p011, CD.Mstcl.HSPA1A.HSP90AA1, 173; p014, CD.Mstcl.HSPA1A.HSP90AA1, 2; p016, CD.Mstcl.HSPA1A.HSP90AA1, 4; p018,
CD.Mstcl.HSPA1A.HSP90AA1, 0; p019, CD.Mstcl.HSPA1A.HSP90AA1, 0; p022, CD.Mstcl.HSPA1A.HSP90AA1, 0; p024, CD.Mstcl.HSPA1A.HSP90AA1, 0; p026,
CD.Mstcl.HSPA1A.HSP90AA1, 0; p027, CD.Mstcl.HSPA1A.HSP90AA1, 3; p031, CD.Mstcl.HSPA1A.HSP90AA1, 1; p032, CD.Mstcl.HSPA1A.HSP90AA1, 0; p041,
CD.Mstcl.HSPA1A.HSP90AA1, 4; p042, CD.Mstcl.HSPA1A.HSP90AA1, 4; p048, CD.Mstcl.HSPA1A.HSP90AA1, 88; p011, CD.Mstcl.JCHAIN.IGKC, 8; p014,
CD.Mstcl.JCHAIN.IGKC, 1; p016, CD.Mstcl.JCHAIN.IGKC, 555; p018, CD.Mstcl.JCHAIN.IGKC, 1; p019, CD.Mstcl.JCHAIN.IGKC, 0; p022, CD.Mstcl.JCHAIN.IGKC, 8; p024,
CD.Mstcl.JCHAIN.IGKC, 3; p026, CD.Mstcl.JCHAIN.IGKC, 0; p027, CD.Mstcl.JCHAIN.IGKC, 1; p031, CD.Mstcl.JCHAIN.IGKC, 0; p032, CD.Mstcl.JCHAIN.IGKC, 1; p041,
CD.Mstcl.JCHAIN.IGKC, 1; p042, CD.Mstcl.JCHAIN.IGKC, 3; p048, CD.Mstcl.JCHAIN.IGKC, 10; p011, CD.Mstcl.KLRB1.GNLY, 2; p014, CD.Mstcl.KLRB1.GNLY, 3; p016,
CD.Mstcl.KLRB1.GNLY, 12; p018, CD.Mstcl.KLRB1.GNLY, 3; p019, CD.Mstcl.KLRB1.GNLY, 0; p022, CD.Mstcl.KLRB1.GNLY, 4; p024, CD.Mstcl.KLRB1.GNLY, 3; p026,
CD.Mstcl.KLRB1.GNLY, 12; p027, CD.Mstcl.KLRB1.GNLY, 4; p031, CD.Mstcl.KLRB1.GNLY, 18; p032, CD.Mstcl.KLRB1.GNLY, 0; p041, CD.Mstcl.KLRB1.GNLY, 9; p042,
CD.Mstcl.KLRB1.GNLY, 23; p048, CD.Mstcl.KLRB1.GNLY, 2; p011, CD.Mstcl.NUCB2.HSPA5, 36; p014, CD.Mstcl.NUCB2.HSPA5, 0; p016, CD.Mstcl.NUCB2.HSPA5, 0; p018,
CD.Mstcl.NUCB2.HSPA5, 0; p019, CD.Mstcl.NUCB2.HSPA5, 0; p022, CD.Mstcl.NUCB2.HSPA5, 0; p024, CD.Mstcl.NUCB2.HSPA5, 0; p026, CD.Mstcl.NUCB2.HSPA5, 0; p027,
CD.Mstcl.NUCB2.HSPA5, 0; p031, CD.Mstcl.NUCB2.HSPA5, 0; p032, CD.Mstcl.NUCB2.HSPA5, 0; p041, CD.Mstcl.NUCB2.HSPA5, 1; p042, CD.Mstcl.NUCB2.HSPA5, 0; p048,
CD.Mstcl.NUCB2.HSPA5, 0; p011, CD.Mstcl.TCL1A.TK1, 0; p014, CD.Mstcl.TCL1A.TK1, 0; p016, CD.Mstcl.TCL1A.TK1, 0; p018, CD.Mstcl.TCL1A.TK1, 0; p019,
CD.Mstcl.TCL1A.TK1, 0; p022, CD.Mstcl.TCL1A.TK1, 0; p024, CD.Mstcl.TCL1A.TK1, 0; p026, CD.Mstcl.TCL1A.TK1, 43; p027, CD.Mstcl.TCL1A.TK1, 0; p031,
CD.Mstcl.TCL1A.TK1, 1; p032, CD.Mstcl.TCL1A.TK1, 1; p041, CD.Mstcl.TCL1A.TK1, 0; p042, CD.Mstcl.TCL1A.TK1, 1; p048, CD.Mstcl.TCL1A.TK1, 0; p011,
CD.Mstcl.TPSAB1.HPGDS, 3; p014, CD.Mstcl.TPSAB1.HPGDS, 0; p016, CD.Mstcl.TPSAB1.HPGDS, 18; p018, CD.Mstcl.TPSAB1.HPGDS, 6; p019, CD.Mstcl.TPSAB1.HPGDS, 0;
p022, CD.Mstcl.TPSAB1.HPGDS, 1; p024, CD.Mstcl.TPSAB1.HPGDS, 9; p026, CD.Mstcl.TPSAB1.HPGDS, 3; p027, CD.Mstcl.TPSAB1.HPGDS, 0; p031, CD.Mstcl.TPSAB1.HPGDS,
7; p032, CD.Mstcl.TPSAB1.HPGDS, 0; p041, CD.Mstcl.TPSAB1.HPGDS, 4; p042, CD.Mstcl.TPSAB1.HPGDS, 18; p048, CD.Mstcl.TPSAB1.HPGDS, 7; p011,
CD.Mstcl.TRBC1.IFI44L, 1; p014, CD.Mstcl.TRBC1.IFI44L, 0; p016, CD.Mstcl.TRBC1.IFI44L, 0; p018, CD.Mstcl.TRBC1.IFI44L, 1; p019, CD.Mstcl.TRBC1.IFI44L, 1; p022,
CD.Mstcl.TRBC1.IFI44L, 0; p024, CD.Mstcl.TRBC1.IFI44L, 0; p026, CD.Mstcl.TRBC1.IFI44L, 41; p027, CD.Mstcl.TRBC1.IFI44L, 1; p031, CD.Mstcl.TRBC1.IFI44L, 3; p032,
CD.Mstcl.TRBC1.IFI44L, 0; p041, CD.Mstcl.TRBC1.IFI44L, 2; p042, CD.Mstcl.TRBC1.IFI44L, 3; p048, CD.Mstcl.TRBC1.IFI44L, 0; p011, CD.Myeloid. MKI67.IGKC, 6; p014,
CD.Myeloid.MKI67.IGKC, 3; p016, CD.Myeloid.MKI67.IGKC, 10; p018, CD.Myeloid.MKI67.IGKC, 0; p019, CD.Myeloid.MKI67.IGKC, 4; p022, CD.Myeloid.MKI67.IGKC, 0; p024,
CD.Myeloid.MKI67.IGKC, 2; p026, CD.Myeloid.MKI67.IGKC, 2; p027, CD.Myeloid.MKI67.IGKC, 4; p031, CD.Myeloid.MKI67.IGKC, 4; p032, CD.Myeloid.MKI67.IGKC, 2; p041,
CD.Myeloid.MKI67.IGKC, 2; p042, CD.Myeloid.MKI67.IGKC, 8; p048, CD.Myeloid.MKI67.IGKC, 24; p011, CD.Myeloid.MKI67.PCNA, 14; p014, CD.Myeloid.MKI67.PCNA, 10;
p016, CD.Myeloid.MKI67.PCNA, 4; p018, CD.Myeloid.MKI67.PCNA, 5; p019, CD.Myeloid.MKI67.PCNA, 4; p022, CD.Myeloid.MKI67.PCNA, 2; p024, CD.Myeloid.MKI67.PCNA,
7; p026, CD.Myeloid.MKI67.PCNA, 3; p027, CD.Myeloid.MKI67.PCNA, 1; p031, CD.Myeloid.MKI67.PCNA, 11; p032, CD.Myeloid.MKI67.PCNA, 0; p041,
CD.Myeloid.MKI67.PCNA, 3; p042, CD.Myeloid.MKI67.PCNA, 10; p048, CD.Myeloid.MKI67.PCNA, 7; p011, CD.MyoFibro.MFAP5.SOSTDC1, 1; p014,
CD.MyoFibro.MFAP5.SOSTDC1, 30; p016, CD.MyoFibro.MFAP5.SOSTDC1, 53; p018, CD.MyoFibro.MFAP5.SOSTDC1, 27; p019, CD.MyoFibro.MFAP5.SOSTDC1, 2; p022,
CD.MyoFibro.MFAP5.SOSTDC1, 31; p024, CD.MyoFibro.MFAP5.SOSTDC1, 53; p026, CD.MyoFibro.MFAP5.SOSTDC1, 10; p027, CD.MyoFibro.MFAP5.SOSTDC1, 4; p031,
CD.MyoFibro.MFAP5.SOSTDC1, 19; p032, CD.MyoFibro.MFAP5.SOSTDC1, 4; p041, CD.MyoFibro.MFAP5.SOSTDC1, 22; p042, CD.MyoFibro.MFAP5.SOSTDC1, 42; p048,
CD.MyoFibro.MFAP5.SOSTDC1, 22; p011, CD.NK.CCL3.CD160, 342; p014, CD.NK.CCL3.CD160, 129; p016, CD.NK.CCL3.CD160, 282; p018, CD.NK.CCL3.CD160, 108; p019,
CD.NK.CCL3.CD160, 47; p022, CD.NK.CCL3.CD160, 106; p024, CD.NK.CCL3.CD160, 76; p026, CD.NK.CCL3.CD160, 36; p027, CD.NK.CCL3.CD160, 60; p031,
CD.NK.CCL3.CD160, 460; p032, CD.NK.CCL3.CD160, 118; p041, CD.NK.CCL3.CD160, 65; p042, CD.NK.CCL3.CD160, 86; p048, CD.NK.CCL3.CD160, 91; p011,
CD.NK.GNLY.FCER1G, 2; p014, CD.NK.GNLY.FCER1G, 0; p016, CD.NK.GNLY.FCER1G, 4; p018, CD.NK.GNLY.FCER1G, 21; p019, CD.NK.GNLY.FCER1G, 1; p022,
CD.NK.GNLY.FCER1G, 26; p024, CD.NK.GNLY.FCER1G, 26; p026, CD.NK.GNLY.FCER1G, 1; p027, CD.NK.GNLY.FCER1G, 11; p031, CD.NK.GNLY.FCER1G, 173; p032,
CD.NK.GNLY.FCER1G, 16; p041, CD.NK.GNLY.FCER1G, 14; p042, CD.NK.GNLY.FCER1G, 19; p048, CD.NK.GNLY.FCER1G, 9; p011, CD.NK.GNLY.FCGR3A, 27; p014,
CD.NK.GNLY.FCGR3A, 0; p016, CD.NK.GNLY.FCGR3A, 2; p018, CD.NK.GNLY.FCGR3A, 0; p019, CD.NK.GNLY.FCGR3A, 0; p022, CD.NK.GNLY.FCGR3A, 0; p024,
CD.NK.GNLY.FCGR3A, 0; p026, CD.NK.GNLY.FCGR3A, 0; p027, CD.NK.GNLY.FCGR3A, 1; p031, CD.NK.GNLY.FCGR3A, 0; p032, CD.NK.GNLY.FCGR3A, 0; p041,
CD.NK.GNLY.FCGR3A, 1; p042, CD.NK.GNLY.FCGR3A, 0; p048, CD.NK.GNLY.FCGR3A, 0; p011, CD.NK.GNLY.GZMB, 0; p014, CD.NK.GNLY.GZMB, 4; p016, CD.NK.GNLY.GZMB,
2; p018, CD.NK.GNLY.GZMB, 4; p019, CD.NK.GNLY.GZMB, 3; p022, CD.NK.GNLY.GZMB, 7; p024, CD.NK.GNLY.GZMB, 44; p026, CD.NK.GNLY.GZMB, 5; p027,
CD.NK.GNLY.GZMB, 8; p031, CD.NK.GNLY.GZMB, 16; p032, CD.NK.GNLY.GZMB, 3; p041, CD.NK.GNLY.GZMB, 7; p042, CD.NK.GNLY.GZMB, 4; p048, CD.NK.GNLY.GZMB, 1;
p011, CD.NK.GNLY.IFNG, 3; p014, CD.NK.GNLY.IFNG, 7; p016, CD.NK.GNLY.IFNG, 0; p018, CD.NK.GNLY.IFNG, 5; p019, CD.NK.GNLY.IFNG, 6; p022, CD.NK.GNLY.IFNG, 62;
p024, CD.NK.GNLY.IFNG, 30; p026, CD.NK.GNLY.IFNG, 12; p027, CD.NK.GNLY.IFNG, 6; p031, CD.NK.GNLY.IFNG, 16; p032, CD.NK.GNLY.IFNG, 14; p041, CD.NK.GNLY.IFNG,
24; p042, CD.NK.GNLY.IFNG, 4; p048, CD.NK.GNLY.IFNG, 11; p011, CD.NK.GNLY.KLRC1, 1; p014, CD.NK.GNLY.KLRC1, 0; p016, CD.NK.GNLY.KLRC1, 0; p018,
CD.NK.GNLY.KLRC1, 11; p019, CD.NK.GNLY.KLRC1, 4; p022, CD.NK.GNLY.KLRC1, 131; p024, CD.NK.GNLY.KLRC1, 6; p026, CD.NK.GNLY.KLRC1, 1; p027,
CD.NK.GNLY.KLRC1, 15; p031, CD.NK.GNLY.KLRC1, 7; p032, CD.NK.GNLY.KLRC1, 24; p041, CD.NK.GNLY.KLRC1, 87; p042, CD.NK.GNLY.KLRC1, 13; p048,
CD.NK.GNLY.KLRC1, 2; p011, CD.NK.GNLY.XCL1, 15; p014, CD.NK.GNLY.XCL1, 3; p016, CD.NK.GNLY.XCL1, 9; p018, CD.NK.GNLY.XCL1, 38; p019, CD.NK.GNLY.XCL1, 7; p022,
CD.NK.GNLY.XCL1, 8; p024, CD.NK.GNLY.XCL1, 11; p026, CD.NK.GNLY.XCL1, 3; p027, CD.NK.GNLY.XCL1, 14; p031, CD.NK.GNLY.XCL1, 12; p032, CD.NK.GNLY.XCL1, 6; p041,
CD.NK.GNLY.XCL1, 16; p042, CD.NK.GNLY.XCL1, 65; p048, CD.NK.GNLY.XCL1, 14; p011, CD.NK.MKI67.GZMA, 17; p014, CD.NK.MKI67.GZMA, 9; p016, CD.NK.MKI67.GZMA,
3; p018, CD.NK.MKI67.GZMA, 2; p019, CD.NK.MKI67.GZMA, 0; p022, CD.NK.MKI67.GZMA, 4; p024, CD.NK.MKI67.GZMA, 8; p026, CD.NK.MKI67.GZMA, 3; p027,
CD.NK.MKI67.GZMA, 0; p031, CD.NK.MKI67.GZMA, 33; p032, CD.NK.MKI67.GZMA, 7; p041, CD.NK.MKI67.GZMA, 12; p042, CD.NK.MKI67.GZMA, 4; p048,
CD.NK.MKI67.GZMA, 0; p011, CD.Paneth.DEFA6.ITLN2, 26; p014, CD.Paneth.DEFA6.ITLN2, 140; p016, CD.Paneth.DEFA6.ITLN2, 21; p018, CD.Paneth.DEFA6.ITLN2, 76;
p019, CD.Paneth.DEFA6.ITLN2, 7; p022, CD.Paneth.DEFA6.ITLN2, 12; p024, CD.Paneth.DEFA6.ITLN2, 34; p026, CD.Paneth.DEFA6.ITLN2, 22; p027,
CD.Paneth.DEFA6.ITLN2, 32; p031, CD.Paneth.DEFA6.ITLN2, 137; p032, CD.Paneth.DEFA6.ITLN2, 4; p041, CD.Paneth.DEFA6.ITLN2, 3; p042, CD.Paneth.DEFA6.ITLN2, 6;
p048, CD.Paneth.DEFA6.ITLN2, 129; p011, CD.pDC.IRF7.IL3RA, 26; p014, CD.pDC.IRF7.IL3RA, 15; p016, CD.pDC.IRF7.IL3RA, 6; p018, CD.pDC.IRF7.IL3RA, 25; p019,
CD.pDC.IRF7.IL3RA, 4; p022, CD.pDC.IRF7.IL3RA, 15; p024, CD.pDC.IRF7.IL3RA, 26; p026, CD.pDC.IRF7.IL3RA, 25; p027, CD.pDC.IRF7.IL3RA, 10; p031,
CD.pDC.IRF7.IL3RA, 46; p032, CD.pDC.IRF7.IL3RA, 16; p041, CD.pDC.IRF7.IL3RA, 34; p042, CD.pDC.IRF7.IL3RA, 12; p048, CD.pDC.IRF7.IL3RA, 0; p011, CD.Plsma/IgA,
1045; p014, CD.Plsma/IgA, 82; p016, CD.Plsma/IgA, 992; p018, CD.Plsma/IgA, 800; p019, CD.Plsma/IgA, 78; p022, CD.Plsma/IgA, 685; p024, CD.Plsma/IgA, 2002; p026,
CD.Plsma/IgA, 2; p027, CD.Plsma/IgA, 196; p031, CD.Plsma/IgA, 438; p032, CD.Plsma/IgA, 317; p041, CD.Plsma/IgA, 841; p042, CD.Plsma/IgA, 992; p048, CD.Plsma/IgA,
766; p011, CD.Plsma/IgG, 434; p014, CD.Plsma/IgG, 39; p016, CD.Plsma/IgG, 26; p018, CD.Plsma/IgG, 278; p019, CD.Plsma/IgG, 110; p022, CD.Plsma/IgG, 949; p024,
CD.Plsma/IgG, 397; p026, CD.Plsma/IgG, 61; p027, CD.Plsma/IgG, 84; p031, CD.Plsma/IgG, 356; p032, CD.Plsma/IgG, 381; p041, CD.Plsma/IgG, 326; p042, CD.Plsma/IgG,
67; p048, CD.Plsma/IgG, 108; p011, CD.Plsma/IgM, 334; p014, CD.Plsma/IgM, 38; p016, CD.Plsma/IgM, 330; p018, CD.Plsma/IgM, 57; p019, CD.Plsma/IgM, 12; p022,
CD.Plsma/IgM, 42; p024, CD.Plsma/IgM, 386; p026, CD.Plsma/IgM, 143; p027, CD.Plsma/IgM, 44; p031, CD.Plsma/IgM, 85; p032, CD.Plsma/IgM, 33; p041, CD.Plsma/IgM,
215; p042, CD.Plsma/IgM, 300; p048, CD.Plsma/IgM, 164; p011, CD.Prolif/EC.UBE2C.CCNB2, 9; p014, CD.Prolif/EC.UBE2C.CCNB2, 5; p016, CD.Prolif/EC.UBE2C.CCNB2,
19; p018, CD.Prolif/EC.UBE2C.CCNB2, 428; p019, CD.Prolif/EC.UBE2C.CCNB2, 19; p022, CD.Prolif/EC.UBE2C.CCNB2, 13; p024, CD.Prolif/EC.UBE2C.CCNB2, 1; p026,
CD.Prolif/EC.UBE2C.CCNB2, 16; p027, CD.Prolif/EC.UBE2C.CCNB2, 32; p031, CD.Prolif/EC.UBE2C.CCNB2, 63; p032, CD.Prolif/EC.UBE2C.CCNB2, 7; p041,
CD.Prolif/EC.UBE2C.CCNB2, 2; p042, CD.Prolif/EC.UBE2C.CCNB2, 5; p048, CD.Prolif/EC.UBE2C.CCNB2, 21; p011, CD.Prolif/EC.UBE2C.HMGB2, 32; p014,
CD.Prolif/EC.UBE2C.HMGB2, 16; p016, CD.Prolif/EC.UBE2C.HMGB2, 9; p018, CD.Prolif/EC.UBE2C.HMGB2, 16; p019, CD.Prolif/EC.UBE2C.HMGB2, 5; p022,
CD.Prolif/EC.UBE2C.HMGB2, 34; p024, CD.Prolif/EC.UBE2C.HMGB2, 3; p026, CD.Prolif/EC.UBE2C.HMGB2, 4; p027, CD.Prolif/EC.UBE2C.HMGB2, 25; p031,
CD.Prolif/EC.UBE2C.HMGB2, 61; p032, CD.Prolif/EC.UBE2C.HMGB2, 6; p041, CD.Prolif/EC.UBE2C.HMGB2, 1; p042, CD.Prolif/EC.UBE2C.HMGB2, 4; p048,
CD.Prolif/EC.UBE2C.HMGB2, 30; p011, CD.Prolif/Secretory.REG1B.MGST1, 10; p014, CD.Prolif/Secretory.REG1B.MGST1, 1; p016, CD.Prolif/Secretory.REG1B.MGST1, 0;
p018, CD.Prolif/Secretory.REG1B.MGST1, 3; p019, CD.Prolif/Secretory.REG1B.MGST1, 0; p022, CD.Prolif/Secretory.REG1B.MGST1, 2; p024,
CD.Prolif/Secretory.REG1B.MGST1, 24; p026, CD.Prolif/Secretory.REG1B.MGST1, 11; p027, CD.Prolif/Secretory.REG1B.MGST1, 0; p031,
CD.Prolif/Secretory.REG1B.MGST1, 149; p032, CD.Prolif/Secretory.REG1B.MGST1, 0; p041, CD.Prolif/Secretory.REG1B.MGST1, 1; p042,
CD.Prolif/Secretory.REG1B.MGST1, 0; p048, CD.Prolif/Secretory.REG1B.MGST1, 7; p011, CD.Secretory.GSTA1.REG1B, 0; p014, CD.Secretory.GSTA1.REG1B, 0; p016,
CD.Secretory.GSTA1.REG1B, 0; p018, CD.Secretory.GSTA1.REG1B, 2; p019, CD.Secretory.GSTA1.REG1B, 1; p022, CD.Secretory.GSTA1.REG1B, 0; p024,
CD.Secretory.GSTA1.REG1B, 4; p026, CD.Secretory.GSTA1.REG1B, 2; p027, CD.Secretory.GSTA1.REG1B, 0; p031, CD.Secretory.GSTA1.REG1B, 0; p032,
CD.Secretory.GSTA1.REG1B, 0; p041, CD.Secretory.GSTA1.REG1B, 0; p042, CD.Secretory.GSTA1.REG1B, 1; p048, CD.Secretory.GSTA1.REG1B, 112; p011,
CD.Secretory.REG1B.REG1A, 2; p014, CD.Secretory.REG1B.REG1A, 0; p016, CD.Secretory.REG1B.REG1A, 0; p018, CD.Secretory.REG1B.REG1A, 3; p019,
CD.Secretory.REG1B.REG1A, 1; p022, CD.Secretory.REG1B.REG1A, 0; p024, CD.Secretory.REG1B.REG1A, 14; p026, CD.Secretory.REG1B.REG1A, 6; p027,
CD.Secretory.REG1B.REG1A, 0; p031, CD.Secretory.REG1B.REG1A, 72; p032, CD.Secretory.REG1B.REG1A, 0; p041, CD.Secretory.REG1B.REG1A, 0; p042,
CD.Secretory.REG1B.REG1A, 1; p048, CD.Secretory.REG1B.REG1A, 8; p011, CD.Secretory.REG3G.IFI6, 0; p014, CD.Secretory.REG3G.IFI6, 0; p016, CD.Secretory.REG3G.IFI6,
0; p018, CD.Secretory.REG3G.IFI6, 0; p019, CD.Secretory.REG3G.IFI6, 0; p022, CD.Secretory.REG3G.IFI6, 0; p024, CD.Secretory.REG3G.IFI6, 0; p026,
CD.Secretory.REG3G.IF16, 97; p027, CD.Secretory.REG3G.IFI6, 0; p031, CD.Secretory.REG3G.IFI6, 0; p032, CD.Secretory.REG3G.IFI6, 0; p041, CD.Secretory.REG3G.IFI6, 0;
p042, CD.Secretory.REG3G.IFI6, 0; p048, CD.Secretory.REG3G.IFI6, 0; p011, CD.T.B2M.MT-CO2, 28; p014, CD.T.B2M.MT-CO2, 0; p016, CD.T.B2M.MT-CO2, 1; p018,
CD.T.B2M.MT-CO2, 1; p019, CD.T.B2M.MT-CO2, 0; p022, CD.T.B2M.MT-CO2, 7; p024, CD.T.B2M.MT-CO2, 0; p026, CD.T.B2M.MT-CO2, 0; p027, CD.T.B2M.MT-CO2, 2; p031,
CD.T.B2M.MT-CO2, 15; p032, CD.T.B2M.MT-CO2, 5; p041, CD.T.B2M.MT-CO2, 0; p042, CD.T.B2M.MT-CO2, 3; p048, CD.T.B2M.MT-CO2, 0; p011, CD.T.CARD16.GB2, 3; p014,
CD.T.CARD16.GB2, 2; p016, CD.T.CARD16.GB2, 11; p018, CD.T.CARD16.GB2, 18; p019, CD.T.CARD16.GB2, 8; p022, CD.T.CARD16.GB2, 160; p024, CD.T.CARD16.GB2, 355;
p026, CD.T.CARD16.GB2, 6; p027, CD.T.CARD16.GB2, 68; p031, CD.T.CARD16.GB2, 26; p032, CD.T.CARD16.GB2, 237; p041, CD.T.CARD16.GB2, 166; p042,
CD.T.CARD16.GB2, 131; p048, CD.T.CARD16.GB2, 80; p011, CD.T.CCL20.IFNG, 6; p014, CD.T.CCL20.IFNG, 3; p016, CD.T.CCL20.IFNG, 2; p018, CD.T.CCL20.IFNG, 20; p019,
CD.T.CCL20.IFNG, 2; p022, CD.T.CCL20.IFNG, 12; p024, CD.T.CCL20.IFNG, 19; p026, CD.T.CCL20.IFNG, 13; p027, CD.T.CCL20.IFNG, 23; p031, CD.T.CCL20.IFNG, 19; p032,
CD.T.CCL20.IFNG, 6; p041, CD.T.CCL20.IFNG, 31; p042, CD.T.CCL20.IFNG, 69; p048, CD.T.CCL20.IFNG, 4; p011, CD.T.CCL20.IL22, 0; p014, CD.T.CCL20.IL22, 0; p016,
CD.T.CCL20.IL22, 0; p018, CD.T.CCL20.IL22, 0; p019, CD.T.CCL20.IL22, 0; p022, CD.T.CCL20.IL22, 0; p024, CD.T.CCL20.IL22, 0; p026, CD.T.CCL20.IL22, 207; p027,
CD.T.CCL20.IL22, 0; p031, CD.T.CCL20.IL22, 0; p032, CD.T.CCL20.IL22, 0; p041, CD.T.CCL20.IL22, 2; p042, CD.T.CCL20.IL22, 1; p048, CD.T.CCL20.IL22, 0; p011,
CD.T.CCL20.RORA, 63; p014, CD.T.CCL20.RORA, 27; p016, CD.T.CCL20.RORA, 18; p018, CD.T.CCL20.RORA, 10; p019, CD.T.CCL20.RORA, 4; p022, CD.T.CCL20.RORA, 60;
p024, CD.T.CCL20.RORA, 336; p026, CD.T.CCL20.RORA, 36; p027, CD.T.CCL20.RORA, 41; p031, CD.T.CCL20.RORA, 560; p032, CD.T.CCL20.RORA, 68; p041,
CD.T.CCL20.RORA, 118; p042, CD.T.CCL20.RORA, 95; p048, CD.T.CCL20.RORA, 28; p011, CD.T.CCL4.GMZK, 102; p014, CD.T.CCL4.GMZK, 78; p016, CD.T.CCL4.GMZK, 51;
p018, CD.T.CCL4.GMZK, 195; p019, CD.T.CCL4.GMZK, 165; p022, CD.T.CCL4.GMZK, 105; p024, CD.T.CCL4.GMZK, 182; p026, CD.T.CCL4.GMZK, 1636; p027,
CD.T.CCL4.GMZK, 189; p031, CD.T.CCL4.GMZK, 153; p032, CD.T.CCL4.GMZK, 98; p041, CD.T.CCL4.GMZK, 94; p042, CD.T.CCL4.GMZK, 502; p048, CD.T.CCL4.GMZK, 150;
p011, CD.T.CCR7.GPR183, 90; p014, CD.T.CCR7.GPR183, 70; p016, CD.T.CCR7.GPR183, 63; p018, CD.T.CCR7.GPR183, 918; p019, CD.T.CCR7.GPR183, 952; p022,
CD.T.CCR7.GPR183, 210; p024, CD.T.CCR7.GPR183, 179; p026, CD.T.CCR7.GPR183, 1840; p027, CD.T.CCR7.GPR183, 735; p031, CD.T.CCR7.GPR183, 290; p032,
CD.T.CCR7.GPR183, 452; p041, CD.T.CCR7.GPR183, 248; p042, CD.T.CCR7.GPR183, 409; p048, CD.T.CCR7.GPR183, 78; p011, CD.T.CCR7.SELL, 134; p014, CD.T.CCR7.SELL,
6; p016, CD.T.CCR7.SELL, 176; p018, CD.T.CCR7.SELL, 892; p019, CD.T.CCR7.SELL, 2144; p022, CD.T.CCR7.SELL, 417; p024, CD.T.CCR7.SELL, 125; p026, CD.T.CCR7.SELL,
50; p027, CD.T.CCR7.SELL, 789; p031, CD.T.CCR7.SELL, 120; p032, CD.T.CCR7.SELL, 668; p041, CD.T.CCR7.SELL, 223; p042, CD.T.CCR7.SELL, 192; p048, CD.T.CCR7.SELL,
96; p011, CD.T.CD8B.TRGC2, 20; p014, CD.T.CD8B.TRGC2, 1; p016, CD.T.CD8B.TRGC2, 6; p018, CD.T.CD8B.TRGC2, 17; p019, CD.T.CD8B.TRGC2, 72; p022,
CD.T.CD8B.TRGC2, 13; p024, CD.T.CD8B.TRGC2, 19; p026, CD.T.CD8B.TRGC2, 125; p027, CD.T.CD8B.TRGC2, 254; p031, CD.T.CD8B.TRGC2, 17; p032, CD.T.CD8B.TRGC2, 2;
p041, CD.T.CD8B.TRGC2, 370; p042, CD.T.CD8B.TRGC2, 931; p048, CD.T.CD8B.TRGC2, 104; p011, CD.T.CTLA4.CD27, 32; p014, CD.T.CTLA4.CD27, 3; p016,
CD.T.CTLA4.CD27, 5; p018, CD.T.CTLA4.CD27, 2; p019, CD.T.CTLA4.CD27, 5; p022, CD.T.CTLA4.CD27, 2; p024, CD.T.CTLA4.CD27, 0; p026, CD.T.CTLA4.CD27, 5; p027,
CD.T.CTLA4.CD27, 23; p031, CD.T.CTLA4.CD27, 14; p032, CD.T.CTLA4.CD27, 9; p041, CD.T.CTLA4.CD27, 11; p042, CD.T.CTLA4.CD27, 3; p048, CD.T.CTLA4.CD27, 3; p011,
CD.T.CTLA4.IL2RA, 2; p014, CD.T.CTLA4.IL2RA, 5; p016, CD.T.CTLA4.IL2RA, 0; p018, CD.T.CTLA4.IL2RA, 1; p019, CD.T.CTLA4.IL2RA, 5; p022, CD.T.CTLA4.IL2RA, 13; p024,
CD.T.CTLA4.IL2RA, 11; p026, CD.T.CTLA4.IL2RA, 5; p027, CD.T.CTLA4.IL2RA, 20; p031, CD.T.CTLA4.IL2RA, 498; p032, CD.T.CTLA4.IL2RA, 31; p041, CD.T.CTLA4.IL2RA, 9;
p042, CD.T.CTLA4.IL2RA, 5; p048, CD.T.CTLA4.IL2RA, 10; p011, CD.T.EGR1.IFNG, 22; p014, CD.T.EGR1.IFNG, 0; p016, CD.T.EGR1.IFNG, 1; p018, CD.T.EGR1.IFNG, 48; p019,
CD.T.EGR1.IFNG, 17; p022, CD.T.EGR1.IFNG, 1; p024, CD.T.EGR1.IFNG, 19; p026, CD.T.EGR1.IFNG, 26; p027, CD.T.EGR1.IFNG, 87; p031, CD.T.EGR1.IFNG, 2; p032,
CD.T.EGR1.IFNG, 3; p041, CD.T.EGR1.IFNG, 18; p042, CD.T.EGR1.IFNG, 29; p048, CD.T.EGR1.IFNG, 13; p011, CD.T.EGR1.TNF, 17; p014, CD.T.EGR1.TNF, 4; p016,
CD.T.EGR1.TNF, 6; p018, CD.T.EGR1.TNF, 2; p019, CD.T.EGR1.TNF, 8; p022, CD.T.EGR1.TNF, 11; p024, CD.T.EGR1.TNF, 6; p026, CD.T.EGR1.TNF, 29; p027, CD.T.EGR1.TNF,
20; p031, CD.T.EGR1.TNF, 14; p032, CD.T.EGR1.TNF, 3; p041, CD.T.EGR1.TNF, 14; p042, CD.T.EGR1.TNF, 93; p048, CD.T.EGR1.TNF, 8; p011, CD.T.GNLY.CSF2, 21; p014,
CD.T.GNLY.CSF2, 1; p016, CD.T.GNLY.CSF2, 0; p018, CD.T.GNLY.CSF2, 6; p019, CD.T.GNLY.CSF2, 0; p022, CD.T.GNLY.CSF2, 50; p024, CD.T.GNLY.CSF2, 32; p026,
CD.T.GNLY.CSF2, 5; p027, CD.T.GNLY.CSF2, 6; p031, CD.T.GNLY.CSF2, 13; p032, CD.T.GNLY.CSF2, 4; p041, CD.T.GNLY.CSF2, 63; p042, CD.T.GNLY.CSF2, 8; p048,
CD.T.GNLY.CSF2, 0; p011, CD.T.GNLY.CTSW, 0; p014, CD.T.GNLY.CTSW, 0; p016, CD.T.GNLY.CTSW, 1; p018, CD.T.GNLY.CTSW, 20; p019, CD.T.GNLY.CTSW, 4; p022,
CD.T.GNLY.CTSW, 4; p024, CD.T.GNLY.CTSW, 5; p026, CD.T.GNLY.CTSW, 17; p027, CD.T.GNLY.CTSW, 8; p031, CD.T.GNLY.CTSW, 4; p032, CD.T.GNLY.CTSW, 1; p041,
CD.T.GNLY.CTSW, 8; p042, CD.T.GNLY.CTSW, 136; p048, CD.T.GNLY.CTSW, 1; p011, CD.T.GNLY.GZMH, 3; p014, CD.T.GNLY.GZMH, 0; p016, CD.T.GNLY.GZMH, 1; p018,
CD.T.GNLY.GZMH, 2; p019, CD.T.GNLY.GZMH, 2; p022, CD.T.GNLY.GZMH, 2; p024, CD.T.GNLY.GZMH, 7; p026, CD.T.GNLY.GZMH, 15; p027, CD.T.GNLY.GZMH, 4; p031,
CD.T.GNLY.GZMH, 1; p032, CD.T.GNLY.GZMH, 2; p041, CD.T.GNLY.GZMH, 8; p042, CD.T.GNLY.GZMH, 15; p048, CD.T.GNLY.GZMH, 3; p011, CD.T.GNLY.HOPX, 269; p014,
CD.T.GNLY.HOPX, 0; p016, CD.T.GNLY.HOPX, 2; p018, CD.T.GNLY.HOPX, 0; p019, CD.T.GNLY.HOPX, 1; p022, CD.T.GNLY.HOPX, 2; p024, CD.T.GNLY.HOPX, 0; p026,
CD.T.GNLY.HOPX, 0; p027, CD.T.GNLY.HOPX, 0; p031, CD.T.GNLY.HOPX, 1; p032, CD.T.GNLY.HOPX, 0; p041, CD.T.GNLY.HOPX, 3; p042, CD.T.GNLY.HOPX, 1; p048,
CD.T.GNLY.HOPX, 1; p011, CD.T.GNLY.IFNG, 1; p014, CD.T.GNLY.IFNG, 1; p016, CD.T.GNLY.IFNG, 1; p018, CD.T.GNLY.IFNG, 4; p019, CD.T.GNLY.IFNG, 4; p022,
CD.T.GNLY.IFNG, 3; p024, CD.T.GNLY.IFNG, 6; p026, CD.T.GNLY.IFNG, 13; p027, CD.T.GNLY.IFNG, 2; p031, CD.T.GNLY.IFNG, 5; p032, CD.T.GNLY.IFNG, 1; p041,
CD.T.GNLY.IFNG, 5; p042, CD.T.GNLY.IFNG, 86; p048, CD.T.GNLY.IFNG, 1; p011, CD.T.GNLY.IGLC2, 7; p014, CD.T.GNLY.IGLC2, 0; p016, CD.T.GNLY.IGLC2, 1; p018,
CD.T.GNLY.IGLC2, 27; p019, CD.T.GNLY.IGLC2, 2; p022, CD.T.GNLY.IGLC2, 96; p024, CD.T.GNLY.IGLC2, 27; p026, CD.T.GNLY.IGLC2, 6; p027, CD.T.GNLY.IGLC2, 3; p031,
CD.T.GNLY.IGLC2, 6; p032, CD.T.GNLY.IGLC2, 0; p041, CD.T.GNLY.IGLC2, 15; p042, CD.T.GNLY.IGLC2, 15; p048, CD.T.GNLY.IGLC2, 1; p011, CD.T.GNLY.MT-CO2, 14; p014,
CD.T.GNLY.MT-CO2, 2; p016, CD.T.GNLY.MT-CO2, 7; p018, CD.T.GNLY.MT-CO2, 2; p019, CD.T.GNLY.MT-CO2, 0; p022, CD.T.GNLY.MT-CO2, 8; p024, CD.T.GNLY.MT-CO2, 1;
p026, CD.T.GNLY.MT-CO2, 5; p027, CD.T.GNLY.MT-CO2, 6; p031, CD.T.GNLY.MT-CO2, 78; p032, CD.T.GNLY.MT-CO2, 0; p041, CD.T.GNLY.MT-CO2, 1; p042, CD.T.GNLY.MT-
CO2, 8; p048, CD.T.GNLY.MT-CO2, 6; p011, CD.T.GZMA.JAML, 1; p014, CD.T.GZMA.JAML, 7; p016, CD.T.GZMA.JAML, 1; p018, CD.T.GZMA.JAML, 0; p019, CD.T.GZMA.JAML,
0; p022, CD.T.GZMA.JAML, 2; p024, CD.T.GZMA.JAML, 0; p026, CD.T.GZMA.JAML, 2; p027, CD.T.GZMA.JAML, 6; p031, CD.T.GZMA.JAML, 25; p032, CD.T.GZMA.JAML, 1; p041,
CD.T.GZMA.JAML, 3; p042, CD.T.GZMA.JAML, 4; p048, CD.T.GZMA.JAML, 1; p011, CD.T.IFI44L.PTGER4, 21; p014, CD.T.IFI44L.PTGER4, 12; p016, CD.T.IFI44L.PTGER4, 4;
p018, CD.T.IFI44L.PTGER4, 13; p019, CD.T.IFI44L.PTGER4, 62; p022, CD.T.IFI44L.PTGER4, 8; p024, CD.T.IFI44L.PTGER4, 14; p026, CD.T.IFI44L.PTGER4, 641; p027,
CD.T.IFI44L.PTGER4, 301; p031, CD.T.IFI44L.PTGER4, 26; p032, CD.T.IFI44L.PTGER4, 19; p041, CD.T.IFI44L.PTGER4, 55; p042, CD.T.IFI44L.PTGER4, 122; p048,
CD.T.IFI44L.PTGER4, 39; p011, CD.T.IFI6.IRF7, 0; p014, CD.T.IFI6.IRF7, 0; p016, CD.T.IFI6.IRF7, 2; p018, CD.T.IFI6.IRF7, 0; p019, CD.T.IFI6.IRF7, 2; p022, CD.T.IFI6.IRF7,
1; p024, CD.T.IFI6.IRF7, 0; p026, CD.T.IFI6.IRF7, 273; p027, CD.T.IFI6.IRF7, 7; p031, CD.T.IFI6.IRF7, 1; p032, CD.T.IFI6.IRF7, 0; p041, CD.T.IFI6.IRF7, 1; p042, CD.T.IFI6.IRF7,
2; p048, CD.T.IFI6.IRF7, 1; p011, CD.T.KLRB1.LTF, 31; p014, CD.T.KLRB1.LTF, 0; p016, CD.T.KLRB1.LTF, 14; p018, CD.T.KLRB1.LTF, 16; p019, CD.T.KLRB1.LTF, 152; p022,
CD.T.KLRB1.LTF, 72; p024, CD.T.KLRB1.LTF, 22; p026, CD.T.KLRB1.LTF, 46; p027, CD.T.KLRB1.LTF, 166; p031, CD.T.KLRB1.LTF, 16; p032, CD.T.KLRB1.LTF, 22; p041,
CD.T.KLRB1.LTF, 214; p042, CD.T.KLRB1.LTF, 1613; p048, CD.T.KLRB1.LTF, 94; p011, CD.T.LAG3.BATF, 0; p014, CD.T.LAG3.BATF, 0; p016, CD.T.LAG3.BATF, 0; p018,
CD.T.LAG3.BATF, 9; p019, CD.T.LAG3.BATF, 175; p022, CD.T.LAG3.BATF, 1; p024, CD.T.LAG3.BATF, 2; p026, CD.T.LAG3.BATF, 23; p027, CD.T.LAG3.BATF, 31; p031,
CD.T.LAG3.BATF, 4; p032, CD.T.LAG3.BATF, 2; p041, CD.T.LAG3.BATF, 8; p042, CD.T.LAG3.BATF, 46; p048, CD.T.LAG3.BATF, 7; p011, CD.T.MAF.CTLA4, 1; p014,
CD.T.MAF.CTLA4, 0; p016, CD.T.MAF.CTLA4, 4; p018, CD.T.MAF.CTLA4, 2; p019, CD.T.MAF.CTLA4, 17; p022, CD.T.MAF.CTLA4, 24; p024, CD.T.MAF.CTLA4, 65; p026,
CD.T.MAF.CTLA4, 3; p027, CD.T.MAF.CTLA4, 26; p031, CD.T.MAF.CTLA4, 119; p032, CD.T.MAF.CTLA4, 16; p041, CD.T.MAF.CTLA4, 77; p042, CD.T.MAF.CTLA4, 35; p048,
CD.T.MAF.CTLA4, 13; p011, CD.T.MKI67.CCR7, 0; p014, CD.T.MKI67.CCR7, 0; p016, CD.T.MKI67.CCR7, 0; p018, CD.T.MKI67.CCR7, 3; p019, CD.T.MKI67.CCR7, 16; p022,
CD.T.MKI67.CCR7, 0; p024, CD.T.MKI67.CCR7, 0; p026, CD.T.MKI67.CCR7, 1; p027, CD.T.MKI67.CCR7, 0; p031, CD.T.MKI67.CCR7, 0; p032, CD.T.MKI67.CCR7, 0; p041,
CD.T.MKI67.CCR7, 0; p042, CD.T.MKI67.CCR7, 0; p048, CD.T.MKI67.CCR7, 1; p011, CD.T.MKI67.CD38, 0; p014, CD.T.MKI67.CD38, 0; p016, CD.T.MKI67.CD38, 1; p018,
CD.T.MKI67.CD38, 0; p019, CD.T.MKI67.CD38, 1; p022, CD.T.MKI67.CD38, 0; p024, CD.T.MKI67.CD38, 1; p026, CD.T.MKI67.CD38, 3; p027, CD.T.MKI67.CD38, 1; p031,
CD.T.MKI67.CD38, 1; p032, CD.T.MKI67.CD38, 0; p041, CD.T.MKI67.CD38, 0; p042, CD.T.MKI67.CD38, 0; p048, CD.T.MKI67.CD38, 0; p011, CD.T.MKI67.FOXP3, 17; p014,
CD.T.MKI67.FOXP3, 1; p016, CD.T.MKI67.FOXP3, 0; p018, CD.T.MKI67.FOXP3, 18; p019, CD.T.MKI67.FOXP3, 4; p022, CD.T.MKI67.FOXP3, 11; p024, CD.T.MKI67.FOXP3,
19; p026, CD.T.MKI67.FOXP3, 8; p027, CD.T.MKI67.FOXP3, 2; p031, CD.T.MKI67.FOXP3, 20; p032, CD.T.MKI67.FOXP3, 8; p041, CD.T.MKI67.FOXP3, 36; p042,
CD.T.MKI67.FOXP3, 2; p048, CD.T.MKI67.FOXP3, 2; p011, CD.T.MKI67.IFNG, 10; p014, CD.T.MKI67.IFNG, 0; p016, CD.T.MKI67.IFNG, 0; p018, CD.T.MKI67.IFNG, 3; p019,
CD.T.MKI67.IFNG, 0; p022, CD.T.MKI67.IFNG, 16; p024, CD.T.MKI67.IFNG, 11; p026, CD.T.MKI67.IFNG, 0; p027, CD.T.MKI67.IFNG, 2; p031, CD.T.MKI67.IFNG, 17; p032,
CD.T.MKI67.IFNG, 8; p041, CD.T.MKI67.IFNG, 42; p042, CD.T.MKI67.IFNG, 0; p048, CD.T.MKI67.IFNG, 0; p011, CD.T.MKI67.IL22, 18; p014, CD.T.MKI67.IL22, 1; p016,
CD.T.MKI67.IL22, 2; p018, CD.T.MKI67.IL22, 4; p019, CD.T.MKI67.IL22, 0; p022, CD.T.MKI67.IL22, 11; p024, CD.T.MKI67.IL22, 45; p026, CD.T.MKI67.IL22, 6; p027,
CD.T.MKI67.IL22, 5; p031, CD.T.MKI67.IL22, 14; p032, CD.T.MKI67.IL22, 9; p041, CD.T.MKI67.IL22, 14; p042, CD.T.MKI67.IL22, 17; p048, CD.T.MKI67.IL22, 0; p011,
CD.T.MKI67.ITGB7, 0; p014, CD.T.MKI67.ITGB7, 0; p016, CD.T.MKI67.ITGB7, 0; p018, CD.T.MKI67.ITGB7, 1; p019, CD.T.MKI67.ITGB7, 0; p022, CD.T.MKI67.ITGB7, 0; p024,
CD.T.MKI67.ITGB7, 0; p026, CD.T.MKI67.ITGB7, 15; p027, CD.T.MKI67.ITGB7, 0; p031, CD.T.MKI67.ITGB7, 0; p032, CD.T.MKI67.ITGB7, 0; p041, CD.T.MKI67.ITGB7, 0;
p042, CD.T.MKI67.ITGB7, 1; p048, CD.T.MKI67.ITGB7, 0; p011, CD.T.MKI67.MAF, 1; p014, CD.T.MKI67.MAF, 0; p016, CD.T.MKI67.MAF, 1; p018, CD.T.MKI67.MAF, 5; p019,
CD.T.MKI67.MAF, 3; p022, CD.T.MKI67.MAF, 1; p024, CD.T.MKI67.MAF, 1; p026, CD.T.MKI67.MAF, 12; p027, CD.T.MKI67.MAF, 3; p031, CD.T.MKI67.MAF, 0; p032,
CD.T.MKI67.MAF, 0; p041, CD.T.MKI67.MAF, 3; p042, CD.T.MKI67.MAF, 1; p048, CD.T.MKI67.MAF, 0; p011, CD.T.MKI67.MT-CO2, 9; p014, CD.T.MKI67.MT-CO2, 4; p016,
CD.T.MKI67.MT-CO2, 3; p018, CD.T.MKI67.MT-CO2, 15; p019, CD.T.MKI67.MT-CO2, 6; p022, CD.T.MKI67.MT-CO2, 4; p024, CD.T.MKI67.MT-CO2, 3; p026, CD.T.MKI67.MT-
CO2, 9; p027, CD.T.MKI67.MT-CO2, 4; p031, CD.T.MKI67.MT-CO2, 9; p032, CD.T.MKI67.MT-CO2, 2; p041, CD.T.MKI67.MT-CO2, 10; p042, CD.T.MKI67.MT-CO2, 2; p048,
CD.T.MKI67.MT-CO2, 0; p011, CD.T.MKI67.NKG7, 12; p014, CD.T.MKI67.NKG7, 2; p016, CD.T.MKI67.NKG7, 4; p018, CD.T.MKI67.NKG7, 3; p019, CD.T.MKI67.NKG7, 2; p022,
CD.T.MKI67.NKG7, 2; p024, CD.T.MKI67.NKG7, 5; p026, CD.T.MKI67.NKG7, 82; p027, CD.T.MKI67.NKG7, 7; p031, CD.T.MKI67.NKG7, 6; p032, CD.T.MKI67.NKG7, 5; p041,
CD.T.MKI67.NKG7, 8; p042, CD.T.MKI67.NKG7, 13; p048, CD.T.MKI67.NKG7, 3; p011, CD.T.MKI67.RRM2, 0; p014, CD.T.MKI67.RRM2, 0; p016, CD.T.MKI67.RRM2, 0; p018,
CD.T.MKI67.RRM2, 3; p019, CD.T.MKI67.RRM2, 4; p022, CD.T.MKI67.RRM2, 1; p024, CD.T.MKI67.RRM2, 2; p026, CD.T.MKI67.RRM2, 4; p027, CD.T.MKI67.RRM2, 0; p031,
CD.T.MKI67.RRM2, 2; p032, CD.T.MKI67.RRM2, 0; p041, CD.T.MKI67.RRM2, 0; p042, CD.T.MKI67.RRM2, 7; p048, CD.T.MKI67.RRM2, 0; p011, CD.T.MT-CO2.CCR7, 12; p014,
CD.T.MT-CO2.CCR7, 1; p016, CD.T.MT-CO2.CCR7, 0; p018, CD.T.MT-CO2.CCR7, 1; p019, CD.T.MT-CO2.CCR7, 19; p022, CD.T.MT-CO2.CCR7, 3; p024, CD.T.MT-CO2.CCR7, 0;
p026, CD.T.MT-CO2.CCR7, 6; p027, CD.T.MT-CO2.CCR7, 66; p031, CD.T.MT-CO2.CCR7, 5; p032, CD.T.MT-CO2.CCR7, 4; p041, CD.T.MT-CO2.CCR7, 2; p042, CD.T.MT-
CO2.CCR7, 4; p048, CD.T.MT-CO2.CCR7, 5; p011, CD.T.MT-CO2.MT-CO1, 172; p014, CD.T.MT-CO2.MT-CO1, 15; p016, CD.T.MT-CO2.MT-CO1, 66; p018, CD.T.MT-CO2.MT-
CO1, 41; p019, CD.T.MT-CO2.MT-CO1, 64; p022, CD.T.MT-CO2.MT-CO1, 9; p024, CD.T.MT-CO2.MT-CO1, 7; p026, CD.T.MT-CO2.MT-CO1, 23; p027, CD.T.MT-CO2.MT-CO1,
208; p031, CD.T.MT-CO2.MT-CO1, 81; p032, CD.T.MT-CO2.MT-CO1, 45; p041, CD.T.MT-CO2.MT-CO1, 49; p042, CD.T.MT-CO2.MT-CO1, 118; p048, CD.T.MT-CO2.MT-CO1,
137; p011, CD.T.MT-CO2.MT-CO3, 134; p014, CD.T.MT-CO2.MT-CO3, 14; p016, CD.T.MT-CO2.MT-CO3, 63; p018, CD.T.MT-CO2.MT-CO3, 76; p019, CD.T.MT-CO2.MT-CO3,
42; p022, CD.T.MT-CO2.MT-CO3, 48; p024, CD.T.MT-CO2.MT-CO3, 25; p026, CD.T.MT-CO2.MT-CO3, 48; p027, CD.T.MT-CO2.MT-CO3, 85; p031, CD.T.MT-CO2.MT-CO3, 251;
p032, CD.T.MT-CO2.MT-CO3, 31; p041, CD.T.MT-CO2.MT-CO3, 28; p042, CD.T.MT-CO2.MT-CO3, 55; p048, CD.T.MT-CO2.MT-CO3, 51; p011, CD.T.MT-CO2.PTGER4, 35;
p014, CD.T.MT-CO2.PTGER4, 1; p016, CD.T.MT-CO2.PTGER4, 3; p018, CD.T.MT-CO2.PTGER4, 0; p019, CD.T.MT-CO2.PTGER4, 2; p022, CD.T.MT-CO2.PTGER4, 1; p024,
CD.T.MT-CO2.PTGER4, 0; p026, CD.T.MT-CO2.PTGER4, 2; p027, CD.T.MT-CO2.PTGER4, 18; p031, CD.T.MT-CO2.PTGER4, 8; p032, CD.T.MT-CO2.PTGER4, 1; p041, CD.T.MT-
CO2.PTGER4, 3; p042, CD.T.MT-CO2.PTGER4, 5; p048, CD.T.MT-CO2.PTGER4, 10; p011, CD.T.NR3C1.CCR6, 161; p014, CD.T.NR3C1.CCR6, 0; p016, CD.T.NR3C1.CCR6, 0;
p018, CD.T.NR3C1.CCR6, 0; p019, CD.T.NR3C1.CCR6, 0; p022, CD.T.NR3C1.CCR6, 0; p024, CD.T.NR3C1.CCR6, 0; p026, CD.T.NR3C1.CCR6, 0; p027, CD.T.NR3C1.CCR6, 0;
p031, CD.T.NR3C1.CCR6, 0; p032, CD.T.NR3C1.CCR6, 0; p041, CD.T.NR3C1.CCR6, 0; p042, CD.T.NR3C1.CCR6, 0; p048, CD.T.NR3C1.CCR6, 3; p011, CD.T.TIGIT.CTLA4, 398;
p014, CD.T.TIGIT.CTLA4, 0; p016, CD.T.TIGIT.CTLA4, 2; p018, CD.T.TIGIT.CTLA4, 1; p019, CD.T.TIGIT.CTLA4, 1; p022, CD.T.TIGIT.CTLA4, 1; p024, CD.T.TIGIT.CTLA4, 1;
p026, CD.T.TIGIT.CTLA4, 0; p027, CD.T.TIGIT.CTLA4, 1; p031, CD.T.TIGIT.CTLA4, 0; p032, CD.T.TIGIT.CTLA4, 1; p041, CD.T.TIGIT.CTLA4, 9; p042, CD.T.TIGIT.CTLA4, 0;
p048, CD.T.TIGIT.CTLA4, 14; p011, CD.T.TNFRSF18.FOXP3, 21; p014, CD.T.TNFRSF18.FOXP3, 13; p016, CD.T.TNFRSF18.FOXP3, 12; p018, CD.T.TNFRSF18.FOXP3, 66;
p019, CD.T.TNFRSF18.FOXP3, 18; p022, CD.T.TNFRSF18.FOXP3, 42; p024, CD.T.TNFRSF18.FOXP3, 22; p026, CD.T.TNFRSF18.FOXP3, 12; p027, CD.T.TNFRSF18.FOXP3, 32;
p031, CD.T.TNFRSF18.FOXP3, 44; p032, CD.T.TNFRSF18.FOXP3, 29; p041, CD.T.TNFRSF18.FOXP3, 82; p042, CD.T.TNFRSF18.FOXP3, 19; p048, CD.T.TNFRSF18.FOXP3, 33;
p011, CD.T.TNFRSF4.MAF, 8; p014, CD.T.TNFRSF4.MAF, 2; p016, CD.T.TNFRSF4.MAF, 11; p018, CD.T.TNFRSF4.MAF, 8; p019, CD.T.TNFRSF4.MAF, 17; p022,
CD.T.TNFRSF4.MAF, 23; p024, CD.T.TNFRSF4.MAF, 38; p026, CD.T.TNFRSF4.MAF, 29; p027, CD.T.TNFRSF4.MAF, 33; p031, CD.T.TNFRSF4.MAF, 92; p032,
CD.T.TNFRSF4.MAF, 105; p041, CD.T.TNFRSF4.MAF, 41; p042, CD.T.TNFRSF4.MAF, 36; p048, CD.T.TNFRSF4.MAF, 21; p011, CD.T.TRDC.TRGC2, 44; p014,
CD.T.TRDC.TRGC2, 3; p016, CD.T.TRDC.TRGC2, 19; p018, CD.T.TRDC.TRGC2, 25; p019, CD.T.TRDC.TRGC2, 113; p022, CD.T.TRDC.TRGC2, 155; p024, CD.T.TRDC.TRGC2, 83;
p026, CD.T.TRDC.TRGC2, 20; p027, CD.T.TRDC.TRGC2, 89; p031, CD.T.TRDC.TRGC2, 98; p032, CD.T.TRDC.TRGC2, 28; p041, CD.T.TRDC.TRGC2, 52; p042,
CD.T.TRDC.TRGC2, 371; p048, CD.T.TRDC.TRGC2, 26; p011, CD.TA.PLCG2.SOX4, 111; p014, CD.TA.PLCG2.SOX4, 0; p016, CD.TA.PLCG2.SOX4, 9; p018, CD.TA.PLCG2.SOX4,
29; p019, CD.TA.PLCG2.SOX4, 0; p022, CD.TA.PLCG2.SOX4, 34; p024, CD.TA.PLCG2.SOX4, 18; p026, CD.TA.PLCG2.SOX4, 11; p027, CD.TA.PLCG2.SOX4, 24; p031,
CD.TA.PLCG2.SOX4, 94; p032, CD.TA.PLCG2.SOX4, 63; p041, CD.TA.PLCG2.SOX4, 32; p042, CD.TA.PLCG2.SOX4, 2; p048, CD.TA.PLCG2.SOX4, 55

TABLE 11
PCA Loadings for joint Epithelial, Myeloid, T/NK/ILC vectors

| | PC1 | PC2 | PC3 | PC4 | PC5 | PC6 | PC7 |
|---|---|---|---|---|---|---|---|
| CD.Mac.APOE.PTGDS | −0.037 | −0.124 | 0.025 | 0.023 | 0.015 | 0.068 | −0.051 |
| CD.T.CCR7.GPR183 | −0.047 | −0.120 | −0.041 | 0.042 | −0.038 | −0.032 | −0.053 |
| CD.T.MKI67.RRM2 | −0.027 | −0.100 | 0.037 | −0.065 | 0.020 | 0.075 | −0.044 |
| CD.Fibro.TNFSF11.CD24 | −0.077 | −0.100 | 0.004 | 0.078 | 0.003 | 0.022 | −0.057 |
| CD.Mcell.CCL23.SPIB | −0.085 | −0.096 | −0.090 | −0.026 | −0.032 | −0.015 | −0.020 |
| CD.B/DZ.RGS13.AICDA | −0.044 | −0.095 | 0.026 | −0.092 | 0.000 | 0.060 | −0.021 |
| CD.Fibro.IFI6.IFI44L | −0.069 | −0.091 | −0.112 | −0.020 | −0.036 | −0.023 | −0.022 |
| CD.B/DZ.AICDA.PLK1 | −0.024 | −0.090 | 0.019 | −0.108 | 0.010 | 0.078 | −0.008 |
| CD.B/GC.KIAA0101.GINS2 | −0.042 | −0.087 | 0.050 | −0.086 | 0.008 | 0.058 | −0.023 |
| CD.T.MKI67.MAF | −0.037 | −0.087 | −0.088 | −0.002 | −0.078 | 0.023 | 0.006 |
| CD.cDC2.CLEC10A.CD1E | −0.083 | −0.087 | 0.056 | 0.060 | −0.014 | 0.006 | −0.058 |
| CD.T.CCL4.GMZK | −0.049 | −0.084 | −0.111 | −0.070 | −0.034 | −0.011 | −0.011 |
| CD.Mac/cDC1.MKI67.IDO1 | −0.050 | −0.084 | −0.136 | −0.025 | −0.015 | 0.004 | −0.018 |
| CD.Mstcl.TRBC1.IFI44L | −0.056 | −0.083 | −0.132 | −0.030 | −0.033 | −0.007 | −0.017 |
| CD.EC.IFI6.IFI27 | −0.054 | −0.081 | −0.132 | −0.020 | −0.037 | −0.010 | −0.007 |
| CD.Fibro.CCL19.IRF7 | −0.065 | −0.080 | −0.101 | −0.033 | −0.049 | −0.034 | −0.032 |
| CD.Enteroendocrine.ONECUT3.CCK | −0.017 | −0.079 | −0.010 | 0.101 | −0.008 | 0.054 | −0.072 |
| CD.T.MKI67.ITGB7 | −0.054 | −0.079 | −0.134 | −0.034 | −0.032 | 0.002 | −0.004 |
| CD.Plsma/IgA.RRM2.TOP2A | 0.094 | −0.079 | 0.070 | −0.037 | −0.005 | 0.048 | −0.002 |
| CD.B.IFI44L.ISG15 | −0.051 | −0.079 | −0.137 | −0.031 | −0.033 | −0.012 | −0.010 |
| CD.EC.TCL1A.IFI6 | −0.051 | −0.079 | −0.137 | −0.030 | −0.032 | −0.005 | −0.009 |
| CD.Fibro.CCL21.CCL19 | −0.061 | −0.078 | 0.051 | 0.054 | −0.036 | −0.001 | −0.061 |
| CD.Mstcl.TCL1A.TK1 | −0.049 | −0.078 | −0.139 | −0.031 | −0.031 | −0.010 | −0.006 |
| CD.B.IGLC3.IFI44L | −0.049 | −0.077 | −0.138 | −0.031 | −0.033 | −0.007 | −0.004 |
| CD.B.IFI44L.IGLC2 | −0.049 | −0.077 | −0.139 | −0.031 | −0.032 | −0.010 | −0.008 |
| CD.T.CCL20.IL22 | −0.049 | −0.077 | −0.139 | −0.033 | −0.035 | −0.007 | −0.005 |
| CD.Secretory.REG3G.IFI6 | −0.050 | −0.077 | −0.139 | −0.032 | −0.032 | −0.006 | −0.005 |
| CD.B/GC.RRM2.TK1 | −0.050 | −0.077 | −0.139 | −0.032 | −0.032 | −0.006 | −0.005 |
| CD.EC.ARHGDIG.SULT1E1 | −0.023 | −0.077 | −0.139 | −0.012 | 0.002 | −0.047 | −0.037 |
| CD.T.IFI6.IRF7 | −0.052 | −0.076 | −0.137 | −0.033 | −0.033 | −0.011 | −0.004 |
| CD.Endth.IFI6.ISG15 | −0.050 | −0.076 | −0.139 | −0.032 | −0.032 | −0.007 | −0.006 |
| CD.ILC.KRT81.IL22 | −0.049 | −0.076 | −0.138 | −0.032 | −0.036 | −0.009 | −0.005 |
| CD.B.HCK.DUSP4 | −0.049 | −0.076 | −0.140 | −0.033 | −0.033 | −0.004 | −0.006 |
| CD.Mstcl.FOS.JUN | 0.021 | −0.075 | 0.064 | −0.077 | −0.036 | −0.006 | −0.048 |
| CD.Fibro.AGT.F3 | 0.017 | −0.074 | 0.029 | 0.079 | 0.004 | 0.045 | −0.018 |
| CD.B.UBE2C.KIAA0101 | −0.059 | −0.074 | −0.027 | −0.080 | −0.022 | 0.056 | −0.050 |
| CD.EC.FABP1.ADIRF | −0.072 | −0.074 | 0.079 | 0.079 | −0.005 | −0.016 | −0.066 |
| CD.Goblet.HES6.COLCA2 | 0.085 | −0.072 | −0.025 | −0.012 | 0.076 | 0.056 | −0.015 |
| CD.B/GC.TYMS.MKI67 | −0.051 | −0.072 | 0.066 | 0.026 | −0.001 | 0.033 | −0.046 |
| CD.Endth/Lymph.CCL21.PROX1 | 0.067 | −0.066 | 0.065 | 0.014 | 0.073 | 0.049 | −0.077 |
| CD.T.IFI44L.PTGER4 | −0.064 | −0.064 | −0.084 | −0.046 | −0.045 | −0.060 | −0.042 |
| CD.Fibro.CHI3L1.CXCL3 | 0.088 | −0.064 | −0.004 | −0.024 | 0.022 | 0.033 | 0.023 |
| CD.T.MKI67.NKG7 | −0.039 | −0.064 | −0.141 | −0.051 | −0.045 | −0.019 | −0.008 |
| CD.T.LAG3.BATF | −0.074 | −0.064 | 0.083 | 0.023 | −0.020 | −0.002 | −0.065 |
| CD.B/GC.RRM2.TYMS | −0.066 | −0.064 | 0.070 | 0.043 | −0.010 | 0.019 | −0.051 |
| CD.Fibro/fDC.FDCSP.CXCL13 | −0.062 | −0.063 | 0.064 | 0.049 | −0.010 | 0.017 | −0.047 |
| CD.Mstcl.KLRB1.GNLY | 0.038 | −0.063 | −0.066 | −0.125 | 0.025 | −0.002 | −0.026 |
| CD.Mstcl.CTSG.CD69 | 0.004 | −0.061 | 0.074 | −0.076 | 0.009 | −0.064 | −0.110 |
| CD.Fibro.RERGL.NTRK2 | 0.008 | −0.060 | 0.024 | 0.076 | −0.028 | 0.043 | 0.021 |
| CD.T.CCR7.SELL | −0.021 | −0.060 | 0.099 | 0.102 | −0.028 | −0.035 | −0.034 |
| CD.pDC.IRF7.IL3RA | 0.009 | −0.059 | −0.134 | 0.062 | 0.001 | 0.007 | −0.032 |
| CD.EC.SLC28A2.GSTA2 | −0.060 | −0.057 | 0.073 | 0.050 | −0.008 | 0.010 | −0.053 |
| CD.B/GC.CENPE.TOP2A | −0.059 | −0.057 | 0.073 | 0.052 | −0.007 | 0.017 | −0.050 |
| CD.ILC.IL22.KIT | −0.062 | −0.057 | 0.077 | 0.049 | −0.007 | 0.009 | −0.054 |
| CD.T.MKI67.CCR7 | −0.068 | −0.057 | 0.073 | 0.052 | −0.006 | 0.029 | −0.049 |
| CD.B/GC.HIST1H1A.RRM2 | −0.058 | −0.057 | 0.072 | 0.050 | −0.007 | 0.014 | −0.051 |
| CD.B.MKI67/GC.RRM2 | −0.058 | −0.057 | 0.072 | 0.050 | −0.007 | 0.014 | −0.051 |
| CD.Endth/Lymph.EFEMP1.CCL21 | 0.013 | −0.057 | 0.058 | 0.085 | −0.060 | −0.043 | −0.038 |
| CD.cDC2.CD1C.NDRG2 | −0.084 | −0.056 | 0.112 | 0.002 | −0.016 | 0.006 | −0.060 |
| CD.Mcell.CSRP2.SPIB | −0.070 | −0.055 | 0.093 | 0.012 | −0.014 | 0.013 | −0.051 |
| CD.T.MKI67.CD38 | −0.028 | −0.054 | −0.077 | −0.041 | 0.075 | −0.027 | 0.037 |
| CD.Fibro.PCDH18.APOE | 0.085 | −0.052 | 0.034 | −0.036 | 0.058 | 0.047 | −0.004 |
| CD.Fibro.STMN2.FABP5 | −0.046 | −0.051 | 0.047 | 0.093 | 0.019 | 0.117 | −0.008 |
| CD.T.GNLY.GZMH | 0.025 | −0.050 | −0.021 | −0.135 | −0.096 | 0.012 | −0.001 |
| CD.B.IGLC3.RP11-347P51 | −0.062 | −0.048 | 0.078 | 0.045 | −0.018 | 0.013 | −0.061 |
| CD.EC.RBP2.CYP3A4 | −0.077 | −0.047 | 0.103 | 0.013 | −0.011 | −0.052 | −0.070 |
| CD.Plsma/IgG.IGHGP.IGHG2 | 0.067 | −0.046 | 0.024 | 0.113 | −0.030 | −0.054 | −0.074 |
| CD.Fibro.LY6H.PAPPA2 | 0.119 | −0.045 | 0.007 | −0.005 | −0.064 | −0.012 | 0.017 |
| CD.Goblet.ITLN1.CLCA1 | 0.030 | −0.044 | −0.021 | 0.001 | 0.090 | −0.108 | −0.042 |
| CD.Goblet.ITLN1.CLCA1 | 0.030 | −0.044 | −0.021 | 0.001 | 0.090 | −0.108 | −0.042 |
| CD.Mono.FCN1.S100A4 | 0.105 | −0.043 | 0.012 | −0.010 | 0.030 | 0.051 | 0.014 |
| CD.NK.GNLY.GZMB | 0.099 | −0.043 | 0.010 | −0.068 | 0.060 | 0.068 | 0.011 |
| CD.B/DZ.AICDA/CDK1 | 0.036 | −0.042 | 0.036 | −0.129 | 0.036 | 0.085 | 0.019 |
| CD.Endth/Lymph.CCL21.LYVE1 | 0.074 | −0.041 | 0.017 | −0.064 | 0.052 | 0.095 | 0.033 |
| CD.Fibro.MT-ND4L.NEAT1 | 0.098 | −0.041 | −0.012 | 0.034 | 0.053 | −0.060 | −0.014 |
| CD.Fibro.PRELP.OGN | 0.012 | −0.041 | 0.067 | −0.008 | −0.003 | −0.095 | −0.028 |
| CD.Mac.MKI67.APOE | −0.006 | −0.039 | 0.059 | −0.014 | 0.085 | 0.092 | 0.027 |
| CD.Goblet.S100P.FCGBP | 0.030 | −0.039 | 0.034 | 0.079 | 0.028 | 0.108 | 0.040 |
| CD.Mono/cDC2.FCN1.CD1C | 0.009 | −0.039 | 0.065 | 0.048 | −0.043 | −0.108 | 0.059 |
| CD.Fibro.DEFA6.DEFA5 | −0.018 | −0.038 | 0.038 | 0.090 | 0.016 | 0.122 | 0.008 |
| CD.Fibro.F3.SOX6 | 0.072 | −0.038 | 0.019 | 0.038 | 0.011 | −0.084 | −0.017 |
| CD.Glial.SPP1.NOV | 0.024 | −0.038 | −0.071 | −0.034 | −0.116 | −0.027 | −0.014 |
| CD.Mstcl.DEFA5.RPLP2 | −0.036 | −0.037 | 0.051 | 0.091 | 0.018 | 0.109 | −0.006 |
| CD.Mac.AIF1.HBEGF | 0.075 | −0.037 | 0.011 | −0.073 | 0.057 | 0.097 | 0.025 |
| CD.Mac.LYZ.TXN | 0.071 | −0.037 | 0.013 | −0.073 | 0.057 | 0.095 | 0.025 |
| CD.Fibro.RPS4Y1.DEFA5 | −0.026 | −0.037 | 0.038 | 0.093 | 0.022 | 0.117 | 0.007 |
| CD.Fibro.CCL13.CXCL6 | 0.072 | −0.037 | 0.012 | −0.073 | 0.054 | 0.099 | 0.030 |
| CD.Secretory.REG1B.REG1A | 0.066 | −0.036 | −0.001 | −0.094 | 0.109 | 0.067 | −0.052 |
| CD.B/DZ.HIST1H1B.MKI67 | 0.075 | −0.036 | 0.016 | −0.074 | 0.059 | 0.089 | 0.024 |
| CD.EC.MT1H.MT1G | 0.047 | −0.036 | 0.052 | 0.041 | −0.051 | 0.023 | 0.030 |
| CD.Endth/Ven.POSTN.ADGRG6 | 0.088 | −0.035 | 0.008 | −0.068 | 0.056 | 0.092 | 0.024 |
| CD.EC.RBP2.ALPI | −0.022 | −0.035 | 0.031 | 0.095 | 0.023 | 0.105 | 0.009 |
| CD.Mac.C1QB.AIF1 | 0.075 | −0.034 | 0.008 | −0.073 | 0.066 | 0.096 | 0.017 |
| CD.Mac.IGSF6.TXNIP | 0.078 | −0.033 | 0.010 | −0.072 | 0.073 | 0.093 | 0.007 |
| CD.Mac/DC.CXCL10.CLEC4E | 0.112 | −0.033 | 0.005 | −0.049 | 0.090 | 0.044 | −0.026 |
| CD.Prolif/EC.UBE2C.CCNB2 | −0.045 | −0.032 | 0.053 | 0.074 | 0.027 | 0.101 | 0.018 |
| CD.EC.NUPR1.LCN2 | 0.086 | −0.031 | 0.004 | −0.073 | 0.066 | 0.093 | 0.005 |
| CD.Fibro.CXCL14.DEFA5 | −0.023 | −0.031 | 0.030 | 0.084 | 0.026 | 0.121 | 0.009 |
| CD.T.CARD16.GB2 | 0.117 | −0.030 | 0.040 | −0.035 | −0.034 | −0.004 | 0.027 |
| CD.Endth/Ven.CCL23.ACKR1 | −0.051 | −0.030 | 0.055 | 0.094 | 0.015 | 0.116 | −0.012 |
| CD.cDC2.CD1C.AREG | 0.091 | −0.030 | −0.046 | −0.006 | 0.097 | −0.064 | −0.095 |
| CD.Plsma/IgA.AL9287683.RPL39 | −0.046 | −0.029 | 0.084 | 0.018 | 0.014 | 0.083 | −0.017 |
| CD.Endth/Art.SEMA3G.SSUH2 | −0.034 | −0.028 | 0.110 | 0.026 | −0.050 | 0.011 | −0.057 |
| CD.T.TRDC.TRGC2 | 0.046 | −0.028 | 0.088 | −0.062 | −0.045 | −0.020 | −0.021 |
| CD.Plsma/IgA.IGHA1.IGKC | 0.126 | −0.028 | 0.022 | −0.065 | −0.019 | 0.038 | −0.010 |
| CD.T.MKI67.IL22 | 0.117 | −0.026 | 0.007 | −0.068 | 0.014 | 0.073 | 0.004 |
| CD.NK.GNLY.IFNG | 0.106 | −0.025 | 0.019 | 0.008 | −0.068 | −0.013 | 0.021 |
| CD.T.GNLY.IFNG | −0.025 | −0.025 | 0.052 | −0.119 | −0.026 | 0.010 | −0.007 |
| CD.EC.AKR1C1.TSPAN1 | −0.013 | −0.025 | 0.026 | 0.082 | 0.017 | 0.113 | 0.018 |
| CD.T.CCL20.IFNG | 0.062 | −0.025 | 0.051 | −0.100 | −0.065 | −0.004 | −0.030 |
| CD.Endth/Ven.POSTN.ACKR1 | 0.080 | −0.024 | 0.031 | 0.069 | −0.095 | −0.053 | 0.014 |
| CD.Mac/DC.CXCL10.CCL19 | −0.022 | −0.024 | 0.024 | 0.080 | 0.024 | 0.123 | 0.013 |
| CD.Goblet.CENPA.UBE2C | 0.087 | −0.024 | −0.047 | −0.061 | 0.105 | 0.042 | 0.014 |
| CD.T.GNLY.CTSW | −0.033 | −0.024 | 0.059 | −0.105 | −0.031 | 0.015 | −0.006 |
| CD.Fibro.GDF15.EGR1 | 0.054 | −0.024 | 0.004 | 0.074 | −0.029 | −0.087 | 0.013 |
| CD.Fibro.PHGDH.STMN2 | −0.017 | −0.024 | 0.022 | 0.078 | 0.023 | 0.120 | 0.018 |
| CD.T.GNLY.IGLC2 | 0.080 | −0.023 | 0.022 | 0.021 | −0.065 | 0.017 | 0.032 |
| CD.EC.MT1H.PHGDH | −0.019 | −0.023 | 0.021 | 0.077 | 0.025 | 0.120 | 0.017 |
| CD.Mac.SEPP1.CXCL3 | −0.034 | −0.023 | −0.014 | −0.134 | −0.035 | −0.024 | −0.025 |
| CD.B.RPS10.FCER2 | −0.067 | −0.022 | 0.098 | −0.001 | −0.043 | −0.037 | −0.081 |
| CD.Mono/Mac.CXCL10.FCN1 | 0.121 | −0.022 | 0.010 | 0.032 | −0.087 | −0.038 | 0.002 |
| CD.Mac.C1QB.FCGR1A | 0.032 | −0.022 | 0.022 | 0.093 | −0.020 | −0.007 | 0.005 |
| CD.Goblet.S100P.TFF1 | −0.014 | −0.021 | 0.026 | 0.093 | 0.030 | 0.107 | 0.033 |
| CD.Mstcl.AREG.ADCYAP1 | 0.112 | −0.021 | 0.014 | 0.034 | −0.113 | −0.051 | 0.016 |
| CD.EpithStem.LINC00176.RPS4Y1 | −0.052 | −0.020 | 0.057 | 0.025 | 0.000 | −0.060 | −0.061 |
| CD.Mac.C1QB.CD14 | 0.106 | −0.020 | 0.007 | 0.010 | 0.004 | −0.017 | −0.019 |
| CD.EC.GSTA2.TMPRSS15 | −0.067 | −0.019 | 0.088 | −0.035 | −0.012 | −0.059 | −0.059 |
| CD.B/LZ.CCL22.NPW | −0.043 | −0.019 | 0.099 | −0.103 | −0.026 | 0.012 | −0.026 |
| CD.Fibro.IGF2.CXCL6 | 0.057 | −0.019 | 0.023 | 0.041 | −0.066 | −0.014 | 0.026 |
| CD.T.TNFRSF4.MAF | 0.086 | −0.018 | 0.008 | 0.018 | 0.018 | −0.109 | −0.046 |
| CD.Prolif/Secretory.REG1B.MGST1 | 0.088 | −0.018 | −0.033 | −0.064 | 0.105 | 0.061 | −0.062 |
| CD.Mono/cDC2.CLEC10A.AREG | −0.053 | −0.018 | 0.086 | −0.091 | −0.024 | −0.002 | −0.022 |
| CD.T.MKI67.MT-CO2 | 0.063 | −0.017 | −0.043 | 0.062 | −0.043 | 0.040 | −0.022 |
| CD.B/GC.MKI67.RRM2 | −0.030 | −0.017 | −0.140 | −0.023 | −0.052 | −0.015 | −0.088 |
| CD.Mono/Mac.CXCL10.CXCL11 | 0.099 | −0.017 | 0.011 | 0.030 | −0.124 | −0.024 | 0.015 |
| CD.B.IGHG1.IGLC2 | 0.073 | −0.017 | 0.019 | 0.029 | −0.069 | −0.010 | 0.030 |
| CD.EC.ANPEP.DUOX2 | 0.047 | −0.016 | 0.011 | 0.061 | 0.002 | −0.092 | −0.020 |
| CD.EC.LYZ.OLFM4 | 0.093 | −0.016 | 0.010 | 0.025 | −0.112 | −0.018 | 0.025 |
| CD.EC.ADH1C.GSTA1 | −0.053 | −0.016 | 0.058 | 0.023 | 0.002 | −0.067 | −0.054 |
| CD.EC.UBD.IFITM1 | 0.038 | −0.016 | 0.007 | 0.063 | −0.007 | −0.083 | −0.001 |
| CD.Mac.CXCL3.APOC1 | 0.094 | −0.016 | 0.006 | 0.035 | −0.021 | −0.002 | −0.006 |
| CD.EC.OLFM4.MT-ND2 | 0.103 | −0.015 | 0.009 | −0.067 | −0.077 | 0.048 | 0.027 |
| CD.Plsma/IgM.IGHM.JCHAIN | −0.057 | −0.015 | −0.113 | −0.101 | −0.019 | −0.011 | 0.054 |
| CD.NK.GNLY.KLRC1 | 0.092 | −0.015 | 0.021 | 0.031 | −0.114 | −0.033 | 0.019 |
| CD.Mono/Mac.CXCL10.GBP1 | 0.055 | −0.015 | 0.021 | 0.073 | −0.019 | −0.084 | −0.015 |
| CD.T.MAF.CTLA4 | 0.118 | −0.015 | 0.010 | −0.057 | 0.019 | −0.026 | −0.072 |
| CD.B.MT-ND4L.IGHG1 | 0.046 | −0.015 | 0.008 | 0.065 | −0.011 | −0.085 | 0.000 |
| CD.Fibro.CD24.IGHG4 | 0.038 | −0.015 | 0.008 | 0.063 | −0.007 | −0.083 | −0.001 |
| CD.Goblet.BPIFB1.AQP5 | 0.061 | −0.015 | 0.018 | 0.038 | −0.065 | −0.014 | 0.030 |
| CD.Goblet.RETNLB.ITLN1 | 0.105 | −0.014 | 0.012 | −0.044 | −0.108 | 0.017 | 0.001 |
| CD.Fibro.CA12.SFRP2 | 0.061 | −0.014 | 0.018 | 0.037 | −0.065 | −0.015 | 0.030 |
| CD.T.CCL20.RORA | 0.109 | −0.014 | −0.014 | −0.056 | 0.099 | 0.003 | −0.072 |
| CD.Mono.FCN1.LYST | 0.055 | −0.014 | 0.022 | 0.006 | 0.118 | 0.075 | −0.007 |
| CD.EC.LECT1.FAM26F | −0.060 | −0.014 | 0.084 | −0.068 | −0.015 | −0.050 | −0.045 |
| CD.B.IGLC3.IGLC2 | 0.046 | −0.014 | 0.005 | 0.062 | −0.008 | −0.095 | −0.009 |
| CD.Fibro.RAMP1.IGHG1 | 0.066 | −0.014 | 0.018 | 0.036 | −0.074 | −0.016 | 0.032 |
| CD.DC.CXCL10.IDO1 | −0.006 | −0.014 | 0.008 | 0.060 | 0.040 | −0.105 | 0.029 |
| CD.B/GC.RRM2.ZWINT | −0.026 | −0.013 | 0.067 | −0.117 | −0.011 | 0.020 | 0.001 |
| CD.Mstcl.CDK1.KIAA0101 | 0.074 | −0.012 | 0.008 | −0.078 | 0.051 | 0.098 | 0.001 |
| CD.Endth/Lymph.LY6H.CCL21 | −0.042 | −0.011 | 0.073 | −0.103 | −0.026 | 0.002 | −0.011 |
| CD.T.GNLY.CSF2 | 0.116 | −0.011 | 0.007 | −0.010 | −0.106 | 0.014 | 0.007 |
| CD.Mono.CXCL3.FCN1 | 0.121 | −0.011 | 0.012 | −0.008 | −0.084 | −0.009 | 0.022 |
| CD.Mac.FOLR2.LILRB5 | −0.043 | −0.011 | 0.064 | −0.107 | −0.021 | 0.002 | −0.006 |
| CD.T.MKI67.FOXP3 | 0.119 | −0.011 | −0.011 | −0.009 | −0.070 | 0.030 | −0.037 |
| CD.Mono.CXCL10.TNF | 0.089 | −0.010 | 0.007 | 0.023 | −0.106 | −0.021 | 0.021 |
| CD.Mono.S100A8.S100A9 | 0.069 | −0.010 | −0.053 | −0.015 | −0.117 | −0.011 | −0.020 |
| CD.Plsma/IgA.IGHA1.IGLV6-57 | 0.099 | −0.009 | 0.044 | −0.065 | 0.024 | 0.061 | 0.041 |
| CD.T.EGR1.IFNG | −0.035 | −0.008 | 0.042 | −0.023 | −0.024 | −0.005 | −0.054 |
| CD.EC.APOC3.APOA4 | −0.044 | −0.008 | 0.021 | 0.024 | 0.028 | 0.001 | 0.062 |
| CD.Mac/DC.CXCL8.NLRP3 | −0.030 | −0.007 | 0.068 | −0.108 | −0.041 | 0.001 | −0.007 |
| CD.EC.GSTA2.AADAC | −0.026 | −0.007 | 0.020 | 0.014 | −0.003 | −0.096 | −0.045 |
| CD.EC.CA1.TMPRSS15 | 0.091 | −0.006 | 0.010 | 0.063 | −0.102 | −0.079 | 0.004 |
| CD.Mono/Mac.CXCL10.LYZ | 0.086 | −0.005 | 0.000 | 0.020 | −0.102 | −0.061 | −0.011 |
| CD.T.KLRB1.LTF | −0.029 | −0.005 | 0.081 | −0.112 | −0.061 | −0.017 | −0.015 |
| CD.Plsma/IgA.IGLV1-51.IGLV10-54 | 0.071 | −0.004 | 0.070 | 0.024 | −0.010 | −0.118 | −0.014 |
| CD.T.EGR1.TNF | −0.026 | −0.003 | 0.038 | −0.129 | −0.056 | −0.038 | −0.023 |
| CD.T.MKI67.IFNG | 0.116 | −0.002 | −0.003 | −0.002 | −0.095 | −0.017 | −0.023 |
| CD.NK.GNLY.FCER1G | 0.081 | −0.002 | −0.013 | −0.009 | 0.104 | −0.048 | −0.099 |
| CD.B.RPS29.RPS21 | 0.021 | −0.001 | 0.092 | −0.121 | −0.085 | 0.040 | −0.030 |
| CD.Endth/Ven.LAMP3.LIPG | 0.071 | 0.000 | −0.002 | −0.008 | −0.112 | −0.026 | −0.006 |
| CD.T.CD8B.TRGC2 | −0.011 | 0.000 | 0.057 | −0.110 | −0.096 | −0.035 | −0.027 |
| CD.Mac.CXCL2.CXCL3 | 0.057 | 0.002 | 0.015 | −0.031 | −0.132 | −0.016 | −0.018 |
| CD.EC.TMPRSS15.FAM8A1 | 0.060 | 0.002 | −0.029 | 0.017 | 0.111 | −0.069 | −0.098 |
| CD.Fibro.COL6A5.SLITRK6 | 0.041 | 0.003 | −0.016 | 0.002 | 0.116 | −0.083 | −0.123 |
| CD.T.MT-CO2.CCR7 | −0.042 | 0.003 | 0.043 | 0.013 | −0.009 | −0.081 | −0.073 |
| CD.EC.ADH1C.RPS4Y1 | −0.076 | 0.004 | 0.082 | −0.050 | 0.015 | −0.059 | 0.041 |
| CD.Fibro.TNFRSF11B.TFPI2 | 0.059 | 0.004 | −0.004 | −0.019 | −0.110 | −0.013 | −0.006 |
| CD.Endth/Ven.ACKR1.ADGRG6 | 0.047 | 0.004 | 0.038 | 0.053 | 0.026 | −0.055 | −0.098 |
| CD.T.CTLA4.IL2RA | 0.052 | 0.004 | −0.027 | −0.001 | 0.115 | −0.061 | −0.114 |
| CD.Mono.FCN1.AREG | 0.049 | 0.004 | −0.030 | −0.005 | 0.119 | −0.047 | −0.108 |
| CD.EC.SMOC2.ASCL2 | 0.051 | 0.005 | −0.031 | −0.014 | 0.125 | −0.040 | −0.114 |
| CD.Plsma/IgM.IGHM.IGLV2-14 | −0.057 | 0.005 | −0.125 | −0.083 | −0.010 | −0.012 | 0.045 |
| CD.Mac.CXCL2.TNF | 0.058 | 0.005 | −0.004 | −0.020 | −0.110 | −0.014 | −0.006 |
| CD.Fibro.LY6H.PCSK6 | 0.043 | 0.006 | −0.033 | −0.006 | 0.118 | −0.050 | −0.111 |
| CD.Endth/Lymph.EFEMP1.LYVE1 | 0.045 | 0.006 | −0.030 | −0.005 | 0.118 | −0.049 | −0.112 |
| CD.EC.HSPA1B.PSMA2 | 0.045 | 0.006 | −0.030 | −0.005 | 0.118 | −0.049 | −0.112 |
| CD.Fibro.COL6A5.CXCL6 | 0.043 | 0.006 | −0.028 | −0.005 | 0.119 | −0.055 | −0.115 |
| CD.MyoFibro.MFAP5.SOSTDC1 | 0.088 | 0.007 | 0.011 | −0.078 | 0.036 | 0.017 | 0.111 |
| CD.Mstcl.TPSAB1.HPGDS | −0.036 | 0.008 | −0.029 | −0.103 | 0.052 | −0.005 | 0.057 |
| CD.ILC.AREG.AHR | 0.043 | 0.009 | −0.034 | 0.003 | 0.124 | −0.051 | −0.100 |
| CD.EC.APOC3.CUBN | −0.018 | 0.009 | −0.017 | 0.036 | 0.034 | −0.014 | 0.075 |
| CD.Fibro.FIGF.MMP11 | −0.055 | 0.010 | 0.072 | −0.123 | −0.023 | −0.011 | −0.010 |
| CD.EC.CFTR.OLFM4 | −0.014 | 0.011 | −0.020 | 0.036 | 0.034 | −0.016 | 0.078 |
| CD.Glial.COL28A1.SPP1 | −0.016 | 0.011 | −0.020 | 0.034 | 0.034 | −0.014 | 0.078 |
| CD.EC.FABP6.SLC26A3 | −0.016 | 0.012 | −0.020 | 0.034 | 0.034 | −0.014 | 0.078 |
| CD.T.TNFRSF18.FOXP3 | 0.093 | 0.012 | 0.031 | 0.010 | −0.087 | −0.036 | −0.008 |
| CD.EC.APOB.APOC3 | −0.023 | 0.013 | −0.013 | 0.033 | 0.034 | −0.018 | 0.074 |
| CD.B.IGLC2.HLA-DRB1 | 0.053 | 0.013 | 0.045 | −0.057 | −0.077 | −0.053 | −0.049 |
| CD.T.GZMA.JAML | 0.045 | 0.014 | −0.028 | −0.005 | 0.101 | −0.088 | −0.092 |
| CD.Goblet.FCGBP.CLCA1 | −0.016 | 0.014 | −0.021 | 0.034 | 0.033 | −0.012 | 0.077 |
| CD.EC.PLCG2.MTRNR2L8 | −0.017 | 0.015 | −0.021 | 0.034 | 0.035 | −0.014 | 0.081 |
| CD.EC.MIF.MTRNR2L8 | −0.014 | 0.017 | −0.022 | 0.034 | 0.034 | −0.008 | 0.076 |
| CD.ILC.LST1.AREG | −0.035 | 0.019 | 0.085 | −0.110 | −0.029 | −0.017 | −0.009 |
| CD.DC.CCR7.FSCN1 | −0.053 | 0.021 | 0.021 | 0.063 | 0.008 | −0.057 | −0.003 |
| CD.Paneth.DEFA6.ITLN2 | 0.008 | 0.021 | 0.040 | −0.105 | 0.113 | 0.076 | 0.071 |
| CD.EC.FABP6.PLCG2 | −0.044 | 0.023 | 0.032 | −0.021 | 0.008 | −0.101 | −0.053 |
| CD.Plsma/IgA.IGHA2.IGKV1-12 | 0.025 | 0.024 | 0.006 | 0.025 | 0.045 | −0.052 | 0.118 |
| CD.Fibro.WFDC1.PTN | −0.050 | 0.025 | 0.061 | −0.045 | 0.013 | −0.083 | −0.064 |
| CD.Enteroendocrine.TFPI2.TPH1 | 0.047 | 0.028 | −0.081 | 0.022 | −0.121 | 0.068 | −0.036 |
| CD.NK.MKI67.GZMA | 0.104 | 0.028 | −0.048 | 0.002 | 0.062 | −0.034 | −0.074 |
| CD.EC.ADH1C.EDN1 | −0.069 | 0.028 | 0.088 | −0.112 | −0.013 | −0.025 | 0.018 |
| CD.B.FCRL4.OSTN-AS1 | 0.037 | 0.028 | 0.020 | −0.064 | 0.069 | −0.069 | −0.128 |
| CD.T.GNLY.MT-CO2 | 0.042 | 0.029 | −0.031 | −0.012 | 0.112 | −0.058 | −0.095 |
| CD.Fibro.ADAMDEC1.C7 | −0.053 | 0.029 | 0.064 | −0.120 | −0.010 | −0.024 | −0.017 |
| CD.Fibro.OGN.C1QTNF3 | 0.054 | 0.030 | 0.005 | −0.026 | 0.016 | −0.042 | 0.139 |
| CD.NK.GNLY.XCL1 | −0.004 | 0.030 | 0.096 | −0.091 | −0.028 | 0.029 | 0.016 |
| CD.B.IGLC3.HLA-DRB1 | −0.010 | 0.031 | 0.013 | −0.010 | 0.045 | −0.080 | −0.111 |
| CD.EC.GNAT3.TRPM5 | −0.072 | 0.033 | 0.047 | −0.013 | 0.021 | −0.083 | −0.003 |
| CD.Secretory.GSTA1.REG1B | −0.031 | 0.033 | 0.047 | −0.092 | 0.002 | 0.003 | −0.002 |
| CD.Mono/Mac/DC.CXCL8.CLEC10A | −0.041 | 0.038 | 0.071 | −0.043 | −0.012 | −0.051 | −0.045 |
| CD.Plsma/IgA.MT-ND5.MT-ND4L | 0.004 | 0.041 | −0.009 | 0.014 | 0.053 | −0.018 | 0.068 |
| CD.Endth/Ven.CPE.GALNT15 | −0.049 | 0.041 | 0.065 | −0.116 | −0.017 | −0.023 | −0.015 |
| CD.EC.GSTA2.CES3 | −0.028 | 0.041 | 0.003 | 0.005 | 0.044 | −0.048 | 0.134 |
| CD.B.MT-CO2.MT-CO1 | 0.041 | 0.042 | −0.042 | −0.006 | 0.100 | −0.028 | −0.139 |
| CD.Plsma/IgA.IGLV2-14.IGLV3-19 | −0.002 | 0.043 | −0.010 | 0.001 | 0.055 | −0.043 | 0.137 |
| CD.Myeloid.MKI67.PCNA | −0.068 | 0.044 | −0.026 | 0.020 | 0.053 | 0.062 | 0.027 |
| CD.Endth/Lymph.CCL21.DKK3 | −0.019 | 0.044 | −0.012 | 0.001 | 0.048 | −0.042 | 0.141 |
| CD.Fibro.CPM.HSD17B2 | −0.019 | 0.044 | −0.012 | 0.001 | 0.048 | −0.042 | 0.141 |
| CD.B.IGHA2.JCHAIN | −0.017 | 0.045 | −0.011 | −0.002 | 0.050 | −0.040 | 0.141 |
| CD.Fibro.TFPI2.CCL13 | 0.011 | 0.045 | −0.011 | −0.010 | 0.004 | −0.049 | 0.131 |
| CD.Fibro.NPY.HSD17B2 | −0.019 | 0.045 | −0.013 | 0.002 | 0.049 | −0.042 | 0.142 |
| CD.Fibro.PCSK6.FIGF | −0.017 | 0.045 | −0.013 | 0.001 | 0.053 | −0.044 | 0.136 |
| CD.cDC2.FCER1A.CD1C | −0.019 | 0.045 | −0.012 | 0.001 | 0.048 | −0.041 | 0.140 |
| CD.Mstcl.JCHAIN.IGKC | −0.012 | 0.047 | −0.009 | −0.003 | 0.045 | −0.037 | 0.145 |
| CD.cDC1.CLEC9A.IDO1 | −0.060 | 0.048 | −0.011 | 0.081 | 0.072 | −0.078 | 0.006 |
| CD.Mstcl.CD3D.CD2 | −0.020 | 0.049 | 0.000 | 0.095 | −0.007 | 0.142 | −0.054 |
| CD.Glial.XKR4.S100B | 0.040 | 0.049 | −0.042 | 0.000 | 0.096 | −0.019 | −0.142 |
| CD.cDC2.MKI67.CD1C | −0.053 | 0.051 | 0.021 | −0.091 | 0.014 | −0.047 | 0.076 |
| CD.Mono/cDC2.CLEC10A.SDS | −0.031 | 0.051 | 0.028 | −0.055 | −0.005 | −0.025 | −0.004 |
| CD.cDC2.CD1C.CD207 | −0.031 | 0.051 | 0.028 | −0.055 | −0.005 | −0.025 | −0.004 |
| CD.B.IGLC3.JCHAIN | −0.030 | 0.052 | 0.064 | −0.136 | −0.026 | −0.008 | −0.063 |
| CD.Mac.CXCL8.HES1 | −0.035 | 0.052 | 0.032 | −0.056 | −0.003 | −0.027 | −0.004 |
| CD.TA.PLCG2.SOX4 | 0.087 | 0.053 | −0.019 | 0.012 | −0.109 | −0.004 | −0.036 |
| CD.Prolif/EC.UBE2C.HMGB2 | 0.009 | 0.053 | 0.053 | −0.009 | −0.038 | −0.013 | −0.017 |
| CD.Plsma/IgA.IGLL5.IGLV2-14 | 0.006 | 0.055 | 0.082 | −0.090 | −0.015 | 0.018 | 0.018 |
| CD.Endth.MT-CO3.MT-CO2 | 0.018 | 0.056 | 0.057 | 0.067 | −0.006 | −0.129 | 0.041 |
| CD.Endth/Mural.HIGD1B.NDUFA4L2 | −0.032 | 0.057 | −0.041 | 0.040 | 0.090 | 0.072 | −0.082 |
| CD.Goblet.FCGBP.ITLN1 | −0.019 | 0.057 | 0.010 | −0.081 | 0.047 | −0.031 | 0.113 |
| CD.B.ACTB.JCHAIN | −0.066 | 0.058 | 0.003 | −0.040 | −0.012 | −0.085 | −0.019 |
| CD.T.MT-CO2.MT-CO3 | 0.033 | 0.058 | −0.019 | 0.002 | 0.104 | −0.069 | −0.078 |
| CD.DC.LTB.IL22RA2 | −0.088 | 0.062 | 0.065 | 0.014 | 0.019 | 0.087 | −0.064 |
| CD.Myeloid.MKI67.IGKC | −0.086 | 0.062 | 0.029 | −0.032 | 0.034 | −0.077 | 0.073 |
| CD.Goblet.FCGBP.HES6 | −0.054 | 0.065 | −0.015 | −0.044 | 0.029 | −0.056 | 0.082 |
| CD.Goblet.REG4.SPINK4 | 0.069 | 0.066 | −0.041 | −0.026 | 0.019 | 0.100 | −0.006 |
| CD.EC.PLCG2.RP11-727F159 | −0.018 | 0.069 | −0.015 | −0.085 | 0.012 | 0.015 | −0.036 |
| CD.Enteroendocrine.NEUROG3.MLN | 0.051 | 0.070 | −0.051 | 0.016 | −0.097 | 0.041 | −0.080 |
| CD.cDC2.CLEC10A.FCGR2B | −0.070 | 0.075 | 0.040 | −0.048 | −0.028 | −0.019 | −0.066 |
| CD.Enteroendocrine.TPH1.CHGA | 0.079 | 0.076 | −0.028 | −0.007 | −0.097 | 0.012 | 0.019 |
| CD.T.B2M.MT-CO2 | 0.055 | 0.076 | −0.037 | 0.050 | 0.014 | −0.009 | −0.100 |
| CD.Glial.S100B.PLP1 | −0.018 | 0.077 | −0.069 | −0.003 | −0.042 | 0.046 | −0.124 |
| CD.Plsma/IgA.IGKV1-12.IGHA2 | −0.016 | 0.079 | −0.017 | 0.049 | 0.024 | −0.070 | 0.072 |
| CD.Goblet.TFF1.TPSG1 | 0.059 | 0.080 | −0.033 | 0.028 | −0.116 | 0.060 | −0.043 |
| CD.B.CD69.GPR183 | 0.060 | 0.080 | −0.025 | 0.023 | −0.064 | 0.045 | −0.059 |
| CD.NK.CCL3.CD160 | 0.037 | 0.080 | −0.029 | 0.016 | 0.099 | −0.066 | 0.043 |
| CD.T.CTLA4.CD27 | 0.015 | 0.083 | −0.020 | 0.036 | −0.027 | −0.089 | −0.091 |
| CD.Endth/Ven.ADGRG6.ACKR1 | −0.045 | 0.084 | 0.030 | 0.027 | −0.008 | −0.025 | −0.004 |
| CD.Goblet.FCGBP.SPINK4 | 0.008 | 0.089 | −0.016 | 0.002 | 0.006 | −0.018 | 0.105 |
| CD.T.MT-CO2.MT-CO1 | −0.049 | 0.090 | 0.043 | −0.040 | 0.002 | −0.086 | −0.035 |
| CD.EC.MIF.AC0904981 | 0.037 | 0.096 | −0.028 | 0.032 | −0.078 | 0.020 | −0.018 |
| CD.cDC1.CLEC9A.XCR1 | −0.009 | 0.097 | −0.046 | 0.061 | 0.091 | 0.060 | −0.023 |
| CD.Plsma/IgA.HSPA1B.HSPA1A | −0.043 | 0.100 | 0.029 | −0.077 | −0.033 | 0.018 | −0.058 |
| CD.EC.PLCG2.MAFB | 0.026 | 0.101 | −0.021 | −0.038 | −0.026 | 0.021 | −0.048 |
| CD.Fibro.HIGD1B.RGS5 | 0.009 | 0.106 | −0.046 | 0.029 | −0.069 | 0.070 | −0.065 |
| CD.cDC2.ZFAND2A.INHBA | −0.014 | 0.109 | −0.049 | 0.025 | −0.037 | 0.085 | −0.069 |
| CD.cDC2.RALA.CD1C | −0.014 | 0.109 | −0.049 | 0.025 | −0.037 | 0.085 | −0.069 |
| CD.Mstcl.NUCB2.HSPA5 | −0.012 | 0.109 | −0.050 | 0.024 | −0.040 | 0.085 | −0.069 |
| CD.Mono/Mac.CCL4.DDX3Y | −0.015 | 0.110 | −0.051 | 0.027 | −0.034 | 0.084 | −0.064 |
| CD.Mono.FCN1.HSPA1A | −0.014 | 0.110 | −0.050 | 0.024 | −0.038 | 0.084 | −0.066 |
| CD.T.GNLY.HOPX | −0.012 | 0.110 | −0.049 | 0.024 | −0.039 | 0.084 | −0.067 |
| CD.T.NR3C1.CCR6 | −0.016 | 0.112 | −0.048 | 0.022 | −0.037 | 0.084 | −0.069 |
| CD.Mono/Mac/DC.CXCL10.IFIT2 | −0.014 | 0.112 | −0.050 | 0.024 | −0.036 | 0.083 | −0.061 |
| CD.T.TIGIT.CTLA4 | −0.014 | 0.116 | −0.047 | 0.019 | −0.043 | 0.082 | −0.068 |
| CD.EC.MTRNR2L1.MT-ND3 | −0.034 | 0.119 | 0.044 | −0.035 | −0.028 | −0.040 | 0.026 |
| CD.NK.GNLY.FCGR3A | −0.015 | 0.119 | −0.051 | 0.023 | −0.035 | 0.070 | −0.040 |
| CD.T.MT-CO2.PTGER4 | −0.038 | 0.122 | −0.004 | −0.024 | −0.012 | −0.028 | −0.085 |
| CD.Mac.JUN.EGR1 | −0.023 | 0.122 | −0.050 | 0.030 | −0.024 | 0.078 | −0.033 |
| CD.Glial.S100B.SPP1 | −0.012 | 0.125 | −0.017 | −0.037 | −0.076 | 0.022 | −0.065 |
| CD.Glial.S100B.SPP1 | −0.012 | 0.125 | −0.017 | −0.037 | −0.076 | 0.022 | −0.065 |
| CD.Mstcl.HSPA1A.HSP90AA1 | −0.031 | 0.125 | −0.027 | −0.011 | −0.036 | 0.057 | −0.062 |
| CD.Mono.FCN1.EIF4A3 | −0.024 | 0.126 | −0.049 | 0.021 | −0.019 | 0.068 | −0.014 |
| CD.Endth/Cap.CD36.FLT1 | 0.013 | 0.128 | 0.031 | 0.009 | 0.058 | 0.027 | −0.023 |
| CD.Glial.SPP1.NRXN1 | −0.024 | 0.131 | −0.009 | −0.039 | −0.063 | 0.067 | −0.024 |
| CD.Plsma/IgA.IGLC3.IGLC2 | −0.029 | 0.131 | −0.024 | −0.023 | −0.027 | 0.052 | −0.063 |
| CD.Plsma/IgA.IGLC3.IGLC2 | −0.029 | 0.131 | −0.024 | −0.023 | −0.027 | 0.052 | −0.063 |
| CD.Plsma/IgA.IGLC3.IGLC2 | −0.029 | 0.131 | −0.024 | −0.023 | −0.027 | 0.052 | −0.063 |
| | PC8 | PC9 | PC10 | PC11 | PC12 | PC13 | PC14 |
| CD.Mac.APOE.PTGDS | −0.005 | −0.084 | 0.010 | −0.012 | 0.039 | −0.014 | −0.001 |
| CD.T.CCR7.GPR183 | −0.039 | −0.066 | −0.019 | −0.014 | 0.008 | 0.030 | −0.008 |
| CD.T.MKI67.RRM2 | 0.047 | 0.022 | −0.038 | 0.102 | 0.035 | 0.004 | −0.040 |
| CD.Fibro.TNFSF11.CD24 | −0.020 | −0.046 | −0.043 | −0.040 | −0.018 | 0.013 | −0.023 |
| CD.Mcell.CCL23.SPIB | −0.019 | −0.003 | −0.038 | 0.015 | 0.019 | 0.010 | −0.017 |
| CD.B/DZ.RGS13.AICDA | −0.063 | 0.057 | −0.033 | 0.048 | 0.014 | −0.039 | −0.135 |
| CD.Fibro.IFI6.IFI44L | −0.030 | −0.004 | −0.012 | 0.002 | 0.030 | 0.000 | −0.002 |
| CD.B/DZ.AICDA.PLK1 | −0.066 | 0.047 | −0.019 | 0.046 | 0.014 | −0.007 | 0.101 |
| CD.B/GC.KIAA0101.GINS2 | −0.047 | 0.063 | −0.044 | 0.081 | −0.006 | −0.019 | −0.041 |
| CD.T.MKI67.MAF | 0.017 | −0.048 | −0.074 | −0.070 | −0.039 | 0.035 | −0.009 |
| CD.cDC2.CLEC10A.CD1E | −0.054 | 0.016 | −0.062 | −0.015 | −0.028 | −0.002 | −0.045 |
| CD.T.CCL4.GMZK | −0.005 | −0.035 | 0.009 | 0.000 | 0.060 | −0.005 | 0.012 |
| CD.Mac/cDC1.MKI67.IDO1 | −0.001 | −0.013 | −0.034 | −0.001 | 0.018 | −0.021 | 0.006 |
| CD.Mstcl.TRBC1.IFI44L | −0.005 | −0.008 | −0.017 | 0.001 | 0.028 | −0.008 | 0.012 |
| CD.EC.IFI6.IFI27 | −0.007 | −0.013 | −0.018 | −0.006 | 0.048 | 0.015 | 0.030 |
| CD.Fibro.CCL19.IRF7 | −0.017 | −0.025 | −0.016 | −0.065 | 0.011 | 0.063 | −0.005 |
| CD.Enteroendocrine.ONECUT3.CCK | −0.077 | 0.000 | −0.036 | 0.060 | 0.061 | −0.041 | 0.040 |
| CD.T.MKI67.ITGB7 | 0.003 | −0.027 | −0.002 | 0.009 | 0.033 | 0.001 | −0.025 |
| CD.Plsma/IgA.RRM2.TOP2A | 0.030 | 0.002 | −0.084 | 0.024 | 0.023 | 0.000 | −0.010 |
| CD.B.IFI44L.ISG15 | −0.005 | −0.015 | −0.011 | −0.006 | 0.032 | 0.005 | 0.049 |
| CD.EC.TCL1A.IFI6 | −0.009 | −0.012 | −0.010 | 0.001 | 0.037 | −0.006 | 0.019 |
| CD.Fibro.CCL21.CCL19 | −0.046 | 0.069 | −0.084 | −0.030 | −0.040 | −0.066 | −0.016 |
| CD.Mstcl.TCL1A.TK1 | −0.009 | −0.020 | −0.003 | 0.007 | 0.035 | −0.004 | 0.018 |
| CD.B.IGLC3.IFI44L | −0.006 | −0.017 | −0.006 | 0.000 | 0.038 | 0.001 | −0.001 |
| CD.B.IFI44L.IGLC2 | −0.006 | −0.018 | −0.006 | −0.002 | 0.032 | 0.000 | 0.007 |
| CD.T.CCL20.IL22 | −0.005 | −0.017 | −0.006 | 0.000 | 0.032 | −0.004 | 0.031 |
| CD.Secretory.REG3G.IFI6 | −0.006 | −0.017 | −0.006 | 0.000 | 0.036 | −0.002 | 0.001 |
| CD.B/GC.RRM2.TK1 | −0.006 | −0.017 | −0.006 | 0.000 | 0.036 | −0.002 | −0.237 |
| CD.EC.ARHGDIG.SULT1E1 | −0.018 | −0.038 | 0.007 | 0.041 | 0.030 | −0.029 | −0.020 |
| CD.T.IFI6.IRF7 | −0.006 | −0.018 | −0.009 | −0.004 | 0.036 | 0.004 | 0.036 |
| CD.Endth.IFI6.ISG15 | −0.006 | −0.017 | −0.005 | −0.001 | 0.036 | −0.001 | 0.024 |
| CD.ILC.KRT81.IL22 | −0.008 | −0.024 | −0.001 | −0.003 | 0.035 | −0.013 | 0.006 |
| CD.B.HCK.DUSP4 | −0.008 | −0.016 | −0.006 | 0.000 | 0.035 | −0.001 | 0.015 |
| CD.Mstcl.FOS.JUN | −0.101 | 0.042 | −0.014 | −0.094 | 0.002 | 0.026 | −0.032 |
| CD.Fibro.AGT.F3 | −0.022 | −0.137 | 0.033 | −0.048 | −0.019 | 0.096 | 0.040 |
| CD.B.UBE2C.KIAA0101 | −0.074 | 0.085 | −0.037 | 0.024 | −0.014 | −0.043 | −0.010 |
| CD.EC.FABP1.ADIRF | −0.038 | −0.004 | −0.044 | −0.036 | 0.009 | −0.038 | −0.025 |
| CD.Goblet.HES6.COLCA2 | −0.069 | −0.064 | 0.032 | −0.011 | 0.063 | 0.026 | −0.055 |
| CD.B/GC.TYMS.MKI67 | −0.062 | 0.107 | −0.083 | −0.004 | −0.006 | −0.080 | 0.176 |
| CD.Endth/Lymph.CCL21.PROX1 | 0.064 | 0.013 | −0.058 | 0.023 | −0.038 | −0.046 | 0.051 |
| CD.T.IFI44L.PTGER4 | −0.020 | −0.039 | 0.000 | −0.072 | 0.003 | 0.098 | 0.014 |
| CD.Fibro.CHI3L1.CXCL3 | −0.129 | −0.026 | 0.043 | −0.021 | 0.021 | 0.072 | 0.068 |
| CD.T.MKI67.NKG7 | −0.017 | −0.034 | −0.016 | 0.007 | 0.013 | 0.000 | −0.013 |
| CD.T.LAG3.BATF | −0.037 | 0.082 | −0.076 | −0.012 | −0.018 | −0.056 | −0.026 |
| CD.B/GC.RRM2.TYMS | −0.038 | 0.096 | −0.082 | 0.007 | −0.010 | −0.086 | 0.009 |
| CD.Fibro/fDC.FDCSP.CXCL13 | −0.042 | 0.098 | −0.089 | −0.005 | −0.010 | −0.091 | −0.001 |
| CD.Mstcl.KLRB1.GNLY | 0.064 | 0.011 | −0.026 | 0.028 | 0.001 | 0.071 | −0.036 |
| CD.Mstcl.CTSG.CD69 | 0.014 | 0.016 | −0.022 | 0.079 | −0.042 | 0.055 | 0.018 |
| CD.Fibro.RERGL.NTRK2 | 0.090 | −0.015 | −0.026 | −0.059 | 0.141 | 0.092 | −0.007 |
| CD.T.CCR7.SELL | −0.066 | 0.008 | −0.065 | −0.001 | 0.019 | −0.018 | −0.024 |
| CD.pDC.IRF7.IL3RA | 0.024 | −0.045 | 0.043 | 0.033 | −0.064 | 0.046 | −0.038 |
| CD.EC.SLC28A2.GSTA2 | −0.046 | 0.102 | −0.085 | −0.010 | −0.010 | −0.079 | −0.032 |
| CD.B/GC.CENPE.TOP2A | −0.043 | 0.100 | −0.084 | −0.005 | −0.010 | −0.089 | 0.321 |
| CD.ILC.IL22.KIT | −0.046 | 0.099 | −0.084 | −0.011 | −0.012 | −0.072 | −0.048 |
| CD.T.MKI67.CCR7 | −0.035 | 0.077 | −0.071 | −0.024 | 0.006 | −0.112 | −0.003 |
| CD.B/GC.HIST 1H1A.RRM2 | −0.045 | 0.103 | −0.084 | −0.004 | −0.009 | −0.089 | 0.239 |
| CD.B.MKI67/GC.RRM2 | −0.045 | 0.103 | −0.084 | −0.004 | −0.009 | −0.089 | −0.028 |
| CD.Endth/Lymph.EFEMP1.CCL21 | −0.078 | 0.008 | −0.027 | 0.047 | −0.059 | −0.122 | −0.014 |
| CD.cDC2.CD1C.NDRG2 | −0.003 | 0.034 | −0.052 | 0.022 | −0.042 | 0.041 | −0.016 |
| CD.Mcell.CSRP2.SPIB | −0.026 | 0.093 | −0.071 | 0.041 | −0.014 | −0.075 | −0.042 |
| CD.T.MKI67.CD38 | −0.053 | 0.005 | −0.140 | −0.045 | 0.001 | 0.043 | 0.050 |
| CD.Fibro.PCDH18.APOE | −0.133 | 0.021 | 0.007 | −0.016 | 0.031 | 0.005 | −0.050 |
| CD.Fibro.STMN2.FABP5 | 0.047 | −0.073 | −0.014 | −0.023 | −0.021 | −0.040 | 0.002 |
| CD.T.GNLY.GZMH | −0.020 | −0.046 | −0.003 | −0.015 | −0.042 | −0.013 | 0.034 |
| CD.B.IGLC3.RP11-347P51 | −0.046 | 0.098 | −0.084 | −0.014 | −0.018 | −0.080 | −0.024 |
| CD.EC.RBP2.CYP3A4 | −0.054 | 0.048 | −0.036 | −0.009 | −0.029 | 0.057 | −0.074 |
| CD.Plsma/IgG.IGHGP.IGHG2 | −0.008 | 0.025 | −0.001 | 0.046 | 0.080 | −0.007 | −0.038 |
| CD.Fibro.LY6H.PAPPA2 | −0.093 | −0.026 | 0.017 | 0.002 | −0.030 | −0.026 | −0.070 |
| CD.Goblet.ITLN1.CLCA1 | −0.103 | −0.068 | −0.015 | 0.064 | −0.009 | 0.019 | −0.015 |
| CD.Goblet.ITLN1.CLCA1 | −0.103 | −0.068 | −0.015 | 0.064 | −0.009 | 0.019 | −0.015 |
| CD.Mono.FCN1.S100A4 | −0.126 | −0.033 | 0.039 | 0.021 | 0.007 | 0.006 | −0.054 |
| CD.NK.GNLY.GZMB | −0.069 | 0.031 | −0.011 | −0.054 | 0.008 | 0.045 | −0.066 |
| CD.B/DZ.AICDA/CDK1 | −0.052 | 0.012 | 0.018 | 0.057 | −0.005 | 0.055 | 0.546 |
| CD.Endth/Lymph.CCL21.LYVE1 | −0.104 | 0.039 | 0.008 | −0.037 | 0.018 | 0.032 | −0.050 |
| CD.Fibro.MT-ND4L.NEAT1 | −0.071 | 0.020 | 0.084 | 0.076 | −0.022 | −0.049 | 0.004 |
| CD.Fibro.PRELP.OGN | −0.113 | −0.047 | 0.088 | 0.090 | 0.048 | 0.016 | 0.046 |
| CD.Mac.MKI67.APOE | 0.048 | −0.098 | 0.052 | −0.057 | −0.062 | 0.079 | 0.074 |
| CD.Goblet.S100P.FCGBP | 0.013 | −0.135 | 0.036 | −0.003 | 0.027 | 0.024 | 0.057 |
| CD.Mono/cDC2.FCN1.CD1C | 0.027 | 0.015 | −0.074 | −0.042 | −0.031 | 0.137 | −0.020 |
| CD.Fibro.DEFA6.DEFA5 | 0.075 | −0.101 | 0.004 | −0.024 | −0.003 | −0.011 | −0.007 |
| CD.Fibro.F3.SOX6 | −0.115 | −0.028 | 0.085 | 0.073 | −0.005 | −0.055 | −0.006 |
| CD.Glial.SPP1.NOV | 0.023 | −0.024 | −0.033 | −0.080 | −0.126 | −0.023 | 0.030 |
| CD.Mstcl.DEFA5.RPLP2 | 0.045 | −0.111 | 0.013 | −0.040 | −0.022 | −0.013 | 0.012 |
| CD.Mac.AIF1.HBEGF | −0.097 | 0.029 | 0.000 | −0.042 | 0.011 | 0.032 | 0.000 |
| CD.Mac.LYZ.TXN | −0.102 | 0.026 | 0.002 | −0.048 | 0.008 | 0.043 | 0.006 |
| CD.Fibro.RPS4Y1.DEFA5 | 0.057 | −0.112 | 0.014 | −0.015 | −0.017 | −0.019 | 0.004 |
| CD.Fibro.CCL13.CXCL6 | −0.101 | 0.028 | 0.004 | −0.043 | 0.007 | 0.033 | −0.030 |
| CD.Secretory.REG1B.REG1A | −0.031 | 0.027 | −0.015 | 0.006 | 0.006 | −0.002 | 0.012 |
| CD.B/DZ.HIST1H1B.MKI67 | −0.109 | 0.016 | 0.014 | −0.032 | 0.015 | 0.020 | −0.105 |
| CD.EC.MT1H.MT1G | 0.058 | 0.057 | −0.049 | −0.001 | 0.178 | 0.041 | −0.001 |
| CD.Endth/Ven.POSTN.ADGRG6 | −0.079 | 0.046 | 0.006 | −0.044 | 0.012 | 0.033 | −0.012 |
| CD.EC.RBP2.ALPI | 0.046 | −0.131 | 0.031 | −0.007 | −0.020 | −0.017 | 0.003 |
| CD.Mac.C1QB.AIF1 | −0.095 | 0.029 | −0.001 | −0.040 | 0.009 | 0.030 | −0.030 |
| CD.Mac.IGSF6.TXNIP | −0.087 | 0.023 | −0.001 | −0.050 | 0.016 | 0.037 | −0.015 |
| CD.Mac/DC.CXCL10.CLEC4E | −0.062 | 0.007 | −0.009 | −0.010 | 0.019 | 0.025 | 0.013 |
| CD.Prolif/EC.UBE2C.CCNB2 | 0.076 | −0.114 | −0.031 | 0.007 | −0.026 | 0.000 | −0.028 |
| CD.EC.NUPR1.LCN2 | −0.084 | 0.032 | −0.008 | −0.041 | 0.003 | 0.025 | −0.048 |
| CD.Fibro.CXCL14.DEFA5 | 0.072 | −0.116 | 0.016 | −0.022 | −0.017 | −0.009 | −0.004 |
| CD.T.CARD16.GB2 | −0.097 | −0.030 | 0.040 | 0.000 | 0.045 | −0.014 | 0.001 |
| CD.Endth/Ven.CCL23.ACKR1 | 0.047 | −0.081 | −0.007 | −0.039 | −0.027 | −0.005 | −0.001 |
| CD.cDC2.CD1C.AREG | 0.025 | 0.003 | 0.003 | 0.015 | −0.014 | −0.004 | 0.000 |
| CD.Plsma/IgA.AL9287683.RPL39 | 0.097 | −0.122 | 0.015 | 0.018 | −0.024 | 0.039 | 0.007 |
| CD.Endth/Art.SEMA3G.SSUH2 | −0.047 | 0.101 | −0.072 | 0.006 | 0.001 | 0.081 | −0.100 |
| CD.T.TRDC.TRGC2 | 0.069 | 0.036 | −0.040 | 0.079 | 0.100 | 0.082 | −0.042 |
| CD.Plsma/IgA.IGHA1.IGKC | 0.006 | 0.031 | −0.039 | −0.069 | −0.015 | 0.029 | −0.012 |
| CD.T.MKI67.IL22 | −0.075 | 0.017 | −0.012 | 0.002 | 0.001 | 0.045 | 0.019 |
| CD.NK.GNLY.IFNG | 0.040 | 0.039 | −0.005 | −0.022 | 0.145 | 0.018 | 0.016 |
| CD.T.GNLY.IFNG | 0.060 | −0.016 | 0.017 | 0.147 | −0.027 | 0.042 | 0.030 |
| CD.EC.AKR1C1.TSPAN1 | 0.081 | −0.126 | 0.024 | −0.029 | 0.001 | 0.025 | 0.013 |
| CD.T.CCL20.IFNG | 0.068 | −0.050 | −0.002 | 0.019 | −0.089 | 0.066 | −0.014 |
| CD.Endth/Ven.POSTN.ACKR1 | 0.022 | 0.017 | −0.012 | 0.040 | 0.117 | 0.024 | 0.024 |
| CD.Mac/DC.CXCL10.CCL19 | 0.070 | −0.124 | 0.023 | −0.022 | −0.020 | −0.002 | −0.013 |
| CD.Goblet.CENPA.UBE2C | −0.022 | −0.012 | −0.065 | −0.054 | −0.035 | −0.020 | −0.040 |
| CD.T.GNLY.CTSW | 0.070 | −0.039 | 0.022 | 0.138 | −0.035 | 0.056 | −0.029 |
| CD.Fibro.GDF15.EGR1 | −0.082 | −0.052 | 0.069 | 0.114 | 0.060 | −0.020 | −0.001 |
| CD.Fibro.PHGDH.STMN2 | 0.075 | −0.128 | 0.027 | −0.022 | −0.014 | 0.003 | −0.018 |
| CD.T.GNLY.IGLC2 | 0.073 | 0.039 | −0.029 | 0.003 | 0.157 | 0.067 | −0.009 |
| CD.EC.MT1H.PHGDH | 0.073 | −0.129 | 0.027 | −0.022 | −0.019 | 0.001 | 0.019 |
| CD.Mac.SEPP1.CXCL3 | 0.084 | −0.011 | 0.038 | 0.096 | −0.048 | −0.005 | −0.004 |
| CD.B.RPS10.FCER2 | −0.039 | 0.053 | −0.054 | −0.098 | −0.012 | −0.023 | −0.018 |
| CD.Mono/Mac.CXCL10.FCN1 | −0.015 | −0.040 | 0.011 | 0.010 | −0.058 | −0.046 | −0.001 |
| CD.Mac.C1QB.FCGR1A | −0.051 | −0.144 | 0.065 | 0.072 | −0.055 | −0.053 | −0.010 |
| CD.Goblet.S100P.TFF1 | 0.043 | −0.140 | 0.013 | 0.001 | −0.018 | −0.007 | 0.016 |
| CD.Mstcl.AREG.ADCYAP1 | 0.009 | −0.011 | −0.002 | 0.020 | 0.020 | −0.016 | 0.015 |
| CD.EpithStem.LINC00176.RPS4Y1 | −0.042 | −0.004 | −0.029 | −0.117 | −0.029 | 0.172 | 0.060 |
| CD.Mac.C1QB.CD14 | 0.074 | 0.049 | −0.034 | 0.006 | 0.150 | 0.031 | 0.007 |
| CD.EC.GSTA2.TMPRSS15 | −0.015 | −0.003 | −0.014 | −0.029 | −0.036 | 0.171 | −0.029 |
| CD.B/LZ.CCL22.NPW | −0.033 | 0.001 | 0.002 | −0.059 | 0.026 | −0.127 | −0.014 |
| CD.Fibro.IGF2.CXCL6 | 0.074 | 0.057 | −0.032 | 0.014 | 0.175 | 0.058 | 0.050 |
| CD.T.TNFRSF4.MAF | −0.075 | −0.077 | 0.034 | 0.065 | −0.002 | −0.065 | −0.034 |
| CD.Prolif/Secretory.REG1B.MGST1 | −0.028 | 0.030 | −0.026 | −0.025 | 0.002 | −0.004 | −0.023 |
| CD.Mono/cDC2.CLEC10A.AREG | 0.048 | −0.016 | 0.014 | 0.125 | −0.042 | 0.075 | −0.024 |
| CD.T.MKI67.MT-CO2 | 0.092 | −0.030 | −0.087 | −0.046 | −0.124 | −0.020 | −0.065 |
| CD.B/GC.MKI67.RRM2 | −0.009 | −0.035 | −0.011 | −0.035 | −0.003 | −0.064 | 0.207 |
| CD.Mono/Mac.CXCL10.CXCL11 | 0.053 | 0.044 | −0.029 | −0.006 | 0.033 | −0.007 | −0.009 |
| CD.B.IGHG1.IGLC2 | 0.071 | 0.050 | −0.027 | 0.007 | 0.165 | 0.061 | 0.014 |
| CD.EC.ANPEP.DUOX2 | −0.108 | −0.093 | 0.066 | 0.090 | 0.022 | 0.010 | 0.024 |
| CD.EC.LYZ.OLFM4 | 0.072 | 0.034 | −0.033 | −0.012 | 0.086 | 0.022 | 0.019 |
| CD.EC.ADH1C.GSTA1 | −0.047 | 0.003 | −0.040 | −0.114 | −0.029 | 0.168 | −0.011 |
| CD.EC.UBD.IFITM1 | −0.113 | −0.077 | 0.072 | 0.113 | 0.002 | −0.044 | −0.016 |
| CD.Mac.CXCL3.APOC1 | 0.111 | 0.045 | −0.031 | 0.002 | 0.134 | 0.042 | −0.025 |
| CD.EC.OLFM4.MT-ND2 | 0.010 | 0.004 | −0.042 | −0.053 | −0.086 | −0.028 | −0.002 |
| CD.Plsma/IgM.IGHM.JCHAIN | −0.020 | −0.028 | −0.055 | 0.014 | −0.026 | 0.018 | −0.058 |
| CD.NK.GNLY.KLRC1 | 0.072 | 0.022 | −0.029 | −0.007 | 0.077 | 0.027 | 0.008 |
| CD.Mono/Mac.CXCL10.GBP1 | −0.100 | −0.073 | 0.073 | 0.096 | 0.005 | −0.003 | −0.037 |
| CD.T.MAF.CTLA4 | 0.033 | 0.005 | −0.044 | −0.051 | −0.067 | −0.047 | −0.013 |
| CD.B.MT-ND4L.IGHG1 | −0.110 | −0.071 | 0.075 | 0.109 | 0.000 | −0.038 | 0.021 |
| CD.Fibro.CD24.IGHG4 | −0.113 | −0.078 | 0.072 | 0.112 | −0.003 | −0.045 | −0.012 |
| CD.Goblet.BPIFB1.AQP5 | 0.077 | 0.048 | −0.026 | 0.014 | 0.175 | 0.065 | 0.021 |
| CD.Goblet.RETNLB.ITLN1 | 0.049 | 0.011 | −0.036 | −0.036 | −0.065 | −0.012 | −0.030 |
| CD.Fibro.CA12.SFRP2 | 0.077 | 0.049 | −0.026 | 0.015 | 0.175 | 0.065 | 0.009 |
| CD.T.CCL20.RORA | 0.006 | 0.016 | −0.025 | −0.009 | −0.017 | −0.023 | −0.002 |
| CD.Mono.FCN1.LYST | 0.054 | −0.083 | −0.030 | −0.084 | 0.055 | 0.031 | 0.007 |
| CD.EC.LECT1.FAM26F | 0.010 | −0.022 | 0.008 | 0.035 | −0.041 | 0.172 | 0.020 |
| CD.B.IGLC3.IGLC2 | −0.101 | −0.075 | 0.075 | 0.103 | −0.011 | −0.051 | −0.022 |
| CD.Fibro.RAMP1.IGHG1 | 0.081 | 0.047 | −0.031 | 0.009 | 0.162 | 0.059 | 0.033 |
| CD.DC.CXCL10.IDO1 | −0.047 | 0.037 | 0.141 | 0.009 | −0.074 | 0.097 | −0.024 |
| CD.B/GC.RRM2.ZWINT | 0.040 | −0.013 | 0.029 | 0.149 | −0.025 | 0.051 | 0.312 |
| CD.Mstcl.CDK1.KIAA0101 | −0.105 | 0.036 | 0.016 | −0.018 | −0.019 | 0.061 | −0.025 |
| CD.Endth/Lymph.LY6H.CCL21 | 0.055 | −0.015 | 0.022 | 0.145 | −0.037 | 0.046 | −0.027 |
| CD.T.GNLY.CSF2 | 0.049 | 0.031 | −0.042 | −0.040 | 0.003 | 0.014 | 0.016 |
| CD.Mono.CXCL3.FCN1 | 0.068 | 0.042 | −0.037 | −0.037 | 0.021 | −0.001 | −0.038 |
| CD.Mac.FOLR2.LILRB5 | 0.056 | −0.011 | 0.030 | 0.151 | −0.025 | 0.037 | −0.062 |
| CD.T.MKI67.FOXP3 | 0.023 | −0.018 | −0.030 | −0.048 | −0.092 | −0.058 | 0.010 |
| CD.Mono.CXCL10.TNF | 0.090 | 0.043 | −0.042 | −0.013 | 0.083 | 0.024 | 0.039 |
| CD.Mono.S100A8.S100A9 | 0.041 | −0.005 | −0.031 | −0.043 | −0.123 | −0.061 | 0.015 |
| CD.Plsma/IgA.IGHA1.IGLV6-57 | −0.084 | −0.019 | −0.036 | −0.058 | −0.060 | 0.016 | −0.005 |
| CD.T.EGR1.IFNG | −0.032 | −0.083 | 0.010 | −0.147 | −0.044 | 0.164 | −0.007 |
| CD.EC.APOC3.APOA4 | 0.045 | 0.141 | 0.132 | 0.023 | −0.078 | −0.005 | −0.025 |
| CD.Mac/DC.CXCL8.NLRP3 | 0.061 | −0.015 | 0.020 | 0.141 | −0.055 | 0.024 | −0.030 |
| CD.EC.GSTA2.AADAC | −0.060 | −0.057 | 0.019 | −0.092 | −0.020 | 0.197 | −0.006 |
| CD.EC.CA1.TMPRSS15 | −0.036 | −0.037 | 0.029 | 0.059 | 0.002 | −0.019 | 0.041 |
| CD.Mono/Mac.CXCL10.LYZ | −0.006 | −0.042 | 0.000 | 0.003 | −0.122 | −0.083 | 0.034 |
| CD.T.KLRB1.LTF | 0.060 | −0.019 | 0.016 | 0.106 | −0.040 | 0.029 | −0.013 |
| CD.Plsma/IgA.IGLV1-51.IGLV10-54 | −0.088 | −0.088 | −0.016 | 0.008 | 0.001 | −0.027 | −0.071 |
| CD.T.EGR1.TNF | 0.071 | −0.018 | −0.008 | 0.100 | −0.004 | 0.069 | −0.016 |
| CD.T.MKI67.IFNG | 0.040 | 0.001 | −0.037 | −0.036 | −0.081 | −0.043 | −0.007 |
| CD.NK.GNLY.FCER1G | 0.080 | −0.005 | −0.032 | 0.013 | 0.023 | −0.034 | −0.028 |
| CD.B.RPS29.RPS21 | 0.001 | 0.018 | −0.011 | 0.006 | −0.047 | −0.015 | −0.056 |
| CD.Endth/Ven.LAMP3.LIPG | 0.024 | −0.019 | −0.024 | −0.047 | −0.150 | −0.075 | 0.006 |
| CD.T.CD8B.TRGC2 | 0.055 | −0.038 | 0.008 | 0.009 | −0.107 | 0.012 | 0.018 |
| CD.Mac.CXCL2.CXCL3 | 0.061 | 0.015 | −0.031 | −0.018 | −0.117 | −0.073 | 0.000 |
| CD.EC.TMPRSS15.FAM8A1 | 0.079 | 0.018 | −0.009 | 0.039 | 0.007 | −0.039 | 0.028 |
| CD.Fibro.COL6A5.SLITRK6 | 0.048 | −0.014 | −0.024 | −0.014 | −0.022 | 0.035 | 0.060 |
| CD.T.MT-CO2.CCR7 | −0.044 | −0.018 | −0.008 | −0.117 | −0.006 | 0.178 | 0.014 |
| CD.EC.ADH1C.RPS4Y1 | −0.001 | −0.008 | −0.104 | 0.048 | −0.046 | 0.105 | −0.055 |
| CD.Fibro.TNFRSF11B.TFPI2 | 0.036 | −0.011 | −0.032 | −0.064 | −0.160 | −0.072 | −0.013 |
| CD.Endth/Ven.ACKR1.ADGRG6 | −0.072 | −0.118 | −0.021 | 0.041 | −0.077 | 0.037 | 0.084 |
| CD.T.CTLA4.IL2RA | 0.073 | 0.003 | −0.027 | 0.018 | −0.006 | −0.043 | −0.005 |
| CD.Mono.FCN1.AREG | 0.084 | 0.016 | −0.033 | 0.015 | −0.008 | −0.038 | 0.018 |
| CD.EC.SMOC2.ASCL2 | 0.067 | 0.012 | −0.035 | 0.004 | −0.012 | −0.022 | 0.001 |
| CD.Plsma/IgM.IGHM.IGLV2-14 | 0.018 | 0.047 | 0.034 | 0.007 | −0.070 | 0.002 | −0.020 |
| CD.Mac.CXCL2.TNF | 0.036 | −0.012 | −0.031 | −0.066 | −0.158 | −0.075 | −0.012 |
| CD.Fibro.LY6H.PCSK6 | 0.084 | 0.010 | −0.032 | 0.013 | −0.010 | −0.046 | 0.017 |
| CD.Endth/Lymph.EFEMP1.LYVE1 | 0.083 | 0.009 | −0.036 | 0.016 | −0.013 | −0.042 | −0.014 |
| CD.EC.HSPA1B.PSMA2 | 0.083 | 0.009 | −0.036 | 0.016 | −0.013 | −0.042 | −0.014 |
| CD.Fibro.COL6A5.CXCL6 | 0.081 | 0.007 | −0.036 | 0.007 | −0.015 | −0.025 | −0.007 |
| CD.MyoFibro.MFAP5.SOSTDC1 | 0.003 | 0.044 | 0.019 | −0.087 | 0.043 | −0.008 | 0.055 |
| CD.Mstcl.TPSAB1.HPGDS | 0.074 | −0.080 | −0.087 | 0.076 | −0.007 | −0.047 | 0.016 |
| CD.ILC.AREG.AHR | 0.088 | 0.029 | −0.010 | 0.016 | −0.024 | −0.040 | 0.025 |
| CD.EC.APOC3.CUBN | 0.036 | 0.134 | 0.145 | −0.015 | −0.071 | 0.009 | −0.009 |
| CD.Fibro.FIGF.MMP11 | 0.047 | −0.032 | 0.052 | 0.095 | 0.022 | −0.012 | 0.021 |
| CD.EC.CFTR.OLFM4 | 0.035 | 0.128 | 0.151 | −0.011 | −0.071 | 0.010 | 0.023 |
| CD.Glial.COL28A1.SPP1 | 0.038 | 0.130 | 0.149 | −0.014 | −0.070 | 0.011 | −0.028 |
| CD.EC.FABP6.SLC26A3 | 0.038 | 0.129 | 0.149 | −0.014 | −0.070 | 0.011 | 0.018 |
| CD.T.TNFRSF18.FOXP3 | 0.058 | −0.063 | −0.007 | −0.095 | −0.023 | −0.087 | 0.034 |
| CD.EC.APOB.APOC3 | 0.034 | 0.132 | 0.151 | −0.028 | −0.063 | 0.002 | 0.019 |
| CD.B.IGLC2.HLA-DRB1 | −0.017 | −0.035 | 0.003 | −0.147 | −0.066 | 0.060 | 0.037 |
| CD.T.GZMA.JAML | 0.103 | 0.031 | −0.002 | −0.009 | −0.038 | 0.001 | 0.000 |
| CD.Goblet.FCGBP.CLCA1 | 0.037 | 0.130 | 0.150 | −0.014 | −0.069 | 0.010 | 0.003 |
| CD.EC.PLCG2.MTRNR2L8 | 0.037 | 0.129 | 0.146 | −0.014 | −0.070 | 0.010 | 0.041 |
| CD.EC.MIF.MTRNR2L8 | 0.030 | 0.131 | 0.152 | −0.011 | −0.069 | 0.012 | −0.009 |
| CD.ILC.LST1.AREG | 0.036 | −0.057 | 0.078 | 0.053 | 0.062 | −0.064 | −0.021 |
| CD.DC.CCR7.FSCN1 | −0.001 | 0.153 | 0.027 | −0.082 | −0.077 | 0.059 | −0.031 |
| CD.Paneth.DEFA6.ITLN2 | 0.003 | 0.033 | 0.008 | −0.014 | −0.015 | 0.008 | 0.049 |
| CD.EC.FABP6.PLCG2 | −0.040 | −0.057 | 0.061 | −0.152 | 0.070 | 0.030 | 0.077 |
| CD.Plsma/IgA.IGHA2.IGKV1-12 | −0.118 | −0.041 | −0.094 | 0.060 | −0.004 | 0.005 | −0.032 |
| CD.Fibro.WFDC1.PTN | −0.017 | −0.044 | 0.030 | −0.141 | 0.037 | 0.088 | 0.010 |
| CD.Enteroendocrine.TFPI2.TPH1 | 0.035 | −0.010 | −0.014 | −0.028 | −0.085 | −0.018 | 0.002 |
| CD.NK.MKI67.GZMA | 0.045 | 0.029 | −0.020 | 0.040 | −0.078 | −0.052 | 0.017 |
| CD.EC.ADH1C.EDN1 | 0.039 | −0.049 | 0.016 | 0.045 | 0.024 | −0.042 | −0.029 |
| CD.B.FCRL4.OSTN-AS1 | 0.063 | −0.027 | −0.024 | −0.071 | −0.013 | −0.036 | −0.024 |
| CD.T.GNLY.MT-CO2 | 0.095 | 0.008 | −0.055 | 0.011 | 0.028 | −0.032 | 0.015 |
| CD.Fibro.ADAMDEC1.C7 | 0.042 | −0.040 | 0.070 | 0.029 | 0.063 | −0.077 | −0.019 |
| CD.Fibro.OGN.C1QTNF3 | −0.001 | 0.042 | −0.071 | −0.104 | −0.058 | 0.058 | −0.001 |
| CD.NK.GNLY.XCL1 | 0.078 | −0.116 | 0.005 | 0.034 | −0.016 | −0.017 | −0.043 |
| CD.B.IGLC3.HLA-DRB1 | 0.000 | −0.028 | −0.014 | −0.095 | −0.028 | 0.171 | 0.008 |
| CD.EC.GNAT3.TRPM5 | −0.025 | 0.000 | 0.041 | −0.174 | 0.036 | 0.028 | 0.041 |
| CD.Secretory.GSTA1.REG1B | −0.020 | −0.034 | 0.068 | −0.085 | 0.109 | −0.142 | −0.031 |
| CD.Mono/Mac/DC.CXCL8.CLEC10A | −0.042 | −0.027 | 0.058 | −0.077 | 0.126 | −0.116 | 0.028 |
| CD.Plsma/IgA.MT-ND5.MT-ND4L | 0.059 | 0.112 | 0.143 | −0.063 | −0.058 | −0.015 | 0.026 |
| CD.Endth/Ven.CPE.GALNT15 | 0.033 | −0.047 | 0.072 | 0.007 | 0.067 | −0.082 | −0.004 |
| CD.EC.GSTA2.CES3 | −0.013 | −0.014 | −0.156 | 0.002 | −0.002 | −0.018 | 0.039 |
| CD.B.MT-CO2.MT-CO1 | 0.059 | 0.020 | −0.037 | 0.005 | 0.006 | −0.006 | −0.019 |
| CD.Plsma/IgA.IGLV2-14.IGLV3-19 | −0.048 | −0.036 | −0.137 | 0.024 | −0.021 | 0.002 | 0.011 |
| CD.Myeloid.MKI67.PCNA | 0.047 | 0.141 | 0.047 | 0.044 | −0.061 | −0.016 | −0.050 |
| CD.Endth/Lymph.CCL21.DKK3 | −0.008 | −0.025 | −0.147 | 0.018 | −0.018 | −0.010 | 0.001 |
| CD.Fibro.CPM.HSD17B2 | −0.008 | −0.025 | −0.147 | 0.018 | −0.018 | −0.010 | 0.001 |
| CD.B.IGHA2.JCHAIN | −0.011 | −0.025 | −0.146 | 0.015 | −0.015 | −0.011 | 0.019 |
| CD.Fibro.TFPI2.CCL13 | 0.011 | −0.028 | −0.155 | −0.017 | −0.064 | −0.035 | 0.003 |
| CD.Fibro.NPY.HSD17B2 | −0.008 | −0.022 | −0.143 | 0.018 | −0.019 | −0.010 | −0.001 |
| CD.Fibro.PCSK6.FIGF | −0.005 | −0.025 | −0.148 | 0.019 | −0.018 | −0.012 | −0.027 |
| CD.cDC2.FCER1A.CD1C | −0.009 | −0.025 | −0.147 | 0.019 | −0.018 | −0.010 | 0.008 |
| CD.Mstcl.JCHAIN.IGKC | −0.011 | −0.021 | −0.146 | 0.015 | 0.001 | −0.008 | −0.003 |
| CD.cDC1.CLEC9A.IDO1 | −0.006 | 0.082 | −0.045 | 0.065 | −0.040 | 0.064 | −0.006 |
| CD.Mstcl.CD3D.CD2 | −0.033 | −0.061 | 0.019 | 0.025 | −0.009 | 0.038 | −0.031 |
| CD.Glial.XKR4.S100B | 0.049 | 0.020 | −0.043 | 0.014 | −0.013 | 0.023 | 0.007 |
| CD.cDC2.MKI67.CD1C | 0.016 | −0.090 | −0.067 | 0.088 | −0.015 | −0.006 | −0.001 |
| CD.Mono/cDC2.CLEC10A.SDS | −0.007 | −0.041 | 0.068 | −0.111 | 0.114 | −0.153 | −0.018 |
| CD.cDC2.CD1C.CD207 | −0.007 | −0.041 | 0.068 | −0.111 | 0.114 | −0.153 | 0.016 |
| CD.B.IGLC3.JCHAIN | 0.019 | −0.037 | 0.044 | 0.027 | −0.007 | 0.029 | −0.005 |
| CD.Mac.CXCL8.HES1 | 0.002 | −0.043 | 0.079 | −0.115 | 0.104 | −0.142 | 0.018 |
| CD.TA.PLCG2.SOX4 | −0.041 | −0.034 | −0.018 | 0.024 | −0.097 | −0.040 | 0.047 |
| CD.Prolif/EC.UBE2C.HMGB2 | 0.062 | 0.042 | −0.077 | 0.114 | 0.124 | 0.114 | −0.098 |
| CD.Plsma/IgA.IGLL5.IGLV2-14 | 0.003 | −0.124 | −0.045 | 0.011 | −0.086 | 0.002 | 0.021 |
| CD.Endth.MT-CO3.MT-CO2 | −0.014 | −0.024 | 0.017 | −0.073 | 0.090 | 0.050 | 0.066 |
| CD.Endth/Mural.HIGD1B.NDUFA4L2 | 0.083 | −0.047 | −0.030 | −0.056 | −0.034 | 0.013 | −0.008 |
| CD.Goblet.FCGBP.ITLN1 | −0.034 | −0.059 | −0.096 | −0.049 | 0.020 | −0.045 | 0.029 |
| CD.B.ACTB.JCHAIN | −0.019 | 0.013 | 0.044 | −0.139 | −0.028 | 0.111 | 0.002 |
| CD.T.MT-CO2.MT-CO3 | 0.069 | −0.031 | −0.090 | −0.015 | 0.048 | −0.022 | −0.040 |
| CD.DC.LTB.IL22RA2 | 0.006 | −0.018 | −0.077 | 0.036 | 0.008 | −0.023 | −0.064 |
| CD.Myeloid.MKI67.IGKC | −0.045 | 0.004 | −0.075 | −0.036 | 0.022 | −0.069 | 0.005 |
| CD.Goblet.FCGBP.HES6 | −0.016 | −0.061 | −0.058 | −0.089 | 0.078 | −0.089 | 0.057 |
| CD.Goblet.REG4.SPINK4 | −0.112 | 0.028 | −0.050 | 0.019 | 0.006 | 0.038 | −0.021 |
| CD.EC.PLCG2.RP11-727F159 | −0.038 | −0.092 | 0.041 | −0.096 | 0.082 | −0.095 | −0.057 |
| CD.Enteroendocrine.NEUROG3.MLN | 0.035 | 0.006 | −0.050 | −0.019 | −0.093 | −0.002 | −0.015 |
| CD.cDC2.CLEC10A.FCGR2B | −0.034 | −0.032 | 0.044 | −0.104 | 0.089 | 0.003 | 0.049 |
| CD.Enteroendocrine.TPH1.CHGA | −0.029 | −0.025 | −0.054 | −0.075 | −0.069 | −0.027 | 0.011 |
| CD.T.B2M.MT-CO2 | 0.018 | 0.021 | −0.033 | 0.104 | 0.083 | 0.046 | 0.027 |
| CD.Glial.S100B.PLP1 | 0.010 | −0.032 | −0.013 | −0.039 | −0.023 | 0.108 | −0.001 |
| CD.Plsma/IgA.IGKV1-12.IGHA2 | −0.097 | −0.046 | −0.074 | 0.049 | −0.028 | 0.076 | 0.007 |
| CD.Goblet.TFF1.TPSG1 | −0.004 | 0.040 | −0.037 | 0.026 | 0.005 | 0.026 | 0.037 |
| CD.B.CD69.GPR183 | −0.111 | 0.004 | 0.019 | 0.072 | −0.007 | 0.026 | 0.088 |
| CD.NK.CCL3.CD160 | 0.028 | −0.010 | −0.108 | 0.037 | 0.020 | −0.051 | 0.043 |
| CD.T.CTLA4.CD27 | −0.050 | −0.037 | −0.044 | −0.035 | −0.081 | 0.086 | 0.039 |
| CD.Endth/Ven.ADGRG6.ACKR1 | −0.082 | −0.082 | −0.048 | 0.107 | −0.038 | 0.092 | 0.039 |
| CD.Goblet.FCGBP.SPINK4 | −0.038 | −0.029 | −0.147 | 0.050 | −0.007 | 0.012 | −0.014 |
| CD.T.MT-CO2.MT-CO1 | −0.033 | −0.071 | −0.007 | −0.107 | 0.036 | −0.014 | −0.044 |
| CD.EC.MIF.AC0904981 | −0.085 | 0.021 | −0.007 | 0.048 | −0.105 | 0.037 | 0.017 |
| CD.cDC1.CLEC9A.XCR1 | 0.053 | −0.030 | −0.035 | 0.028 | −0.003 | 0.057 | 0.079 |
| CD.Plsma/IgA.HSPA1B.HSPA1A | 0.005 | −0.005 | 0.055 | −0.017 | 0.053 | −0.110 | −0.030 |
| CD.EC.PLCG2.MAFB | −0.113 | −0.053 | 0.045 | −0.023 | 0.053 | −0.071 | 0.009 |
| CD.Fibro.HIGD1B.RGS5 | −0.034 | 0.039 | −0.017 | 0.023 | 0.046 | 0.071 | 0.023 |
| CD.cDC2.ZFAND2A.INHBA | −0.051 | 0.036 | −0.018 | 0.057 | 0.010 | 0.043 | 0.038 |
| CD.cDC2.RALA.CD1C | −0.051 | 0.036 | −0.018 | 0.057 | 0.010 | 0.043 | 0.014 |
| CD.Mstcl.NUCB2.HSPA5 | −0.050 | 0.035 | −0.019 | 0.055 | 0.005 | 0.041 | −0.012 |
| CD.Mono/Mac.CCL4.DDX3Y | −0.048 | 0.044 | −0.008 | 0.057 | 0.005 | 0.044 | −0.002 |
| CD.Mono.FCN1.HSPA1A | −0.050 | 0.035 | −0.021 | 0.056 | 0.007 | 0.040 | 0.000 |
| CD.T.GNLY.HOPX | −0.048 | 0.036 | −0.022 | 0.056 | 0.010 | 0.040 | 0.001 |
| CD.T.NR3C1.CCR6 | −0.051 | 0.033 | −0.014 | 0.051 | 0.016 | 0.034 | −0.027 |
| CD.Mono/Mac/DC.CXCL10.IFIT2 | −0.051 | 0.034 | −0.027 | 0.057 | 0.005 | 0.041 | −0.014 |
| CD.T.TIGIT.CTLA4 | −0.051 | 0.030 | −0.014 | 0.042 | 0.015 | 0.023 | 0.009 |
| CD.EC.MTRNR2L1.MT-ND3 | −0.007 | −0.057 | −0.030 | −0.023 | 0.075 | −0.085 | 0.017 |
| CD.NK.GNLY.FCGR3A | −0.051 | 0.027 | −0.054 | 0.049 | −0.010 | 0.047 | −0.012 |
| CD.T.MT-CO2.PTGER4 | −0.034 | −0.021 | −0.005 | −0.084 | 0.042 | 0.041 | −0.013 |
| CD.Mac.JUN.EGR1 | −0.046 | 0.030 | −0.034 | 0.048 | 0.000 | 0.044 | 0.017 |
| CD.Glial.S100B.SPP1 | −0.025 | −0.017 | −0.010 | −0.062 | −0.023 | −0.027 | 0.007 |
| CD.Glial.S100B.SPP1 | −0.025 | −0.017 | −0.010 | −0.062 | −0.023 | −0.027 | 0.007 |
| CD.Mstcl.HSPA1A.HSP90AA1 | −0.047 | 0.008 | 0.024 | −0.015 | 0.067 | −0.044 | 0.003 |
| CD.Mono.FCN1.EIF4A3 | −0.050 | 0.016 | −0.064 | 0.049 | 0.011 | 0.020 | −0.004 |
| CD.Endth/Cap.CD36.FLT1 | −0.080 | −0.032 | 0.016 | 0.020 | 0.056 | −0.029 | −0.026 |
| CD.Glial.SPP1.NRXN1 | 0.009 | 0.007 | −0.001 | 0.039 | 0.021 | 0.046 | −0.026 |
| CD.Plsma/IgA.IGLC3.IGLC2 | −0.031 | 0.005 | 0.018 | −0.005 | 0.058 | −0.054 | −0.028 |
| CD.Plsma/IgA.IGLC3.IGLC2 | −0.031 | 0.005 | 0.018 | −0.005 | 0.058 | −0.054 | −0.028 |
| CD.Plsma/IgA.IGLC3.IGLC2 | −0.031 | 0.005 | 0.018 | −0.005 | 0.058 | −0.054 | −0.028 |

TABLE 12
Differential composition testing for NOA vs FR, NOA vs PR and PR vs FR categories
of anti-TNF response with CD patients. The method used was Wilcoxon rank sum
test with continuity correction and the alternative method was two.sided.

| Cell | Comp | | | | | |
|---|---|---|---|---|---|---|
| type | label | subset | statistic | p.value | mean.null | mean.altr |
| 2 | NOAvsPR | CD.T.IFI6.IRF7 | 20 | 0.018 | 13369.862 | 141.185 |
| 6 | NOAvsPR | CD.cDC2.CLEC10A.FCGR2B | 20 | 0.015 | 21125.624 | 307.220 |
| 1 | NOAvsRESP | CD.EC.RBP2.ALPI | 20 | 0.015 | 5020.434 | 98.232 |
| 8 | NOAvsRESP | CD.Fibro.CCL19.IRF7 | 20 | 0.018 | 40534.812 | 1677.835 |
| 1 | NOAvsRESP | CD.EC.FABP6.PLCG2 | 20 | 0.018 | 20682.781 | 945.263 |
| 1 | NOAvsPR | CD.EC.GNAT3.TRPM5 | 19 | 0.034 | 8088.674 | 387.911 |
| 8 | NOAvsPR | CD.Fibro.IFI6.IFI44L | 19 | 0.030 | 56794.532 | 3174.603 |
| 8 | NOAvsPR | CD.Fibro.CCL19.IRF7 | 19 | 0.037 | 40534.812 | 3454.013 |
| 6 | NOAvsPR | CD.Mac.FOLR2.LILRB5 | 20 | 0.019 | 12546.643 | 1117.550 |
| 2 | NOAvsPR | CD.T.LAG3.BATF | 20 | 0.016 | 14967.050 | 1965.518 |
| 6 | NOAvsPR | CD.Myeloid.MKI67.IGKC | 20 | 0.016 | 15358.891 | 2187.314 |
| 1 | NOAvsRESP | CD.EC.FABP1.ADIRF | 20 | 0.016 | 55014.890 | 7835.784 |
| 2 | NOAvsPR | CD.T.IFI44L.PTGER4 | 19 | 0.032 | 62653.064 | 10332.762 |
| 8 | RESPvsPR | CD.Glial.SPP1.NRXN1 | 1 | 0.016 | 20474.243 | 5154.846 |
| 2 | RESPvsPR | CD.T.EGR1.TNF | 1 | 0.016 | 9096.243 | 3037.783 |
| 6 | NOAvsRESP | CD.Mac.APOE.PTGDS | 20 | 0.016 | 442550.064 | 181098.617 |
| 9 | RESPvsPR | CD.Endth/Ven.ACKR1.ADGRG6 | 24 | 0.016 | 26764.815 | 64116.523 |
| 8 | RESPvsPR | CD.Fibro.LY6H.PAPPA2 | 23 | 0.031 | 9178.744 | 54679.940 |
| 8 | RESPvsPR | CD.Fibro.AGT.F3 | 23 | 0.036 | 6941.171 | 41355.392 |
| 2 | NOAvsPR | CD.NK.GNLY.FCER1G | 1 | 0.032 | 2751.274 | 16839.853 |
| 2 | NOAvsRESP | CD.T.MKI67.IL22 | 0 | 0.019 | 632.878 | 4173.094 |
| 2 | NOAvsPR | CD.T.MKI67.FOXP3 | 0 | 0.016 | 1158.316 | 8252.129 |
| 6 | NOAvsPR | CD.Mono.FCN1.S100A4 | 0 | 0.016 | 13397.714 | 107730.696 |
| 1 | RESPvsPR | CD.Goblet.HES6.COLCA2 | 22 | 0.045 | 711.744 | 7001.105 |
| 2 | NOAvsPR | CD.T.MKI67.IL22 | 0 | 0.019 | 632.878 | 7851.364 |
| 2 | NOAvsPR | CD.T.GNLY.CSF2 | 0 | 0.019 | 658.351 | 10227.522 |
| 6 | NOAvsPR | CD.Mono/Mac.CXCL10.FCN1 | 0 | 0.018 | 798.722 | 24690.722 |
| 2 | NOAvsPR | CD.NK.MKI67.GZMA | 0 | 0.018 | 137.868 | 4540.136 |
| 3 | RESPvsPR | CD.B/DZ.HIST1H1B.MKI67 | 22 | 0.045 | 254.130 | 9905.256 |
| 2 | NOAvsPR | CD.T.MKI67.IFNG | 0 | 0.018 | 142.857 | 6635.144 |
| 2 | NOAvsRESP | CD.NK.MKI67.GZMA | 0 | 0.018 | 137.868 | 6681.911 |
| 9 | NOAvsPR | CD.Endth/Ven.LAMP3.LIPG | 2 | 0.044 | 0.000 | 70851.581 |
| 1 | NOAvsPR | CD.Goblet.TFF1.TPSG1 | 2 | 0.044 | 0.000 | 14427.968 |
| 6 | NOAvsPR | CD.Mac.CXCL3.APOC1 | 2 | 0.044 | 0.000 | 5546.346 |
| 1 | NOAvsPR | CD.EC.APOB.APOC3 | 17.5 | 0.042 | 3788.094 | 0.000 |
| 1 | NOAvsPR | CD.EC.GSTA2.CES3 | 17.5 | 0.042 | 2501.159 | 0.000 |
| 1 | NOAvsPR | CD.EC.GSTA2.TMPRSS15 | 17.5 | 0.042 | 8239.155 | 0.000 |
| 1 | NOAvsPR | CD.EC.SLC28A2.GSTA2 | 17.5 | 0.042 | 25083.713 | 0.000 |
| 8 | NOAvsPR | CD.Fibro.FIGF.MMP11 | 20 | 0.011 | 22889.519 | 0.000 |
| 1 | NOAvsPR | CD.Mcell.CSRP2.SPIB | 17.5 | 0.042 | 31033.543 | 0.000 |
| 3 | NOAvsRESP | CD.B.IFI44L.IGLC2 | 17.5 | 0.042 | 27809.496 | 0.000 |
| 1 | NOAvsRESP | CD.EC.GSTA2.AADAC | 17.5 | 0.042 | 5427.906 | 0.000 |
| 1 | NOAvsRESP | CD.EC.SLC28A2.GSTA2 | 17.5 | 0.042 | 25083.713 | 0.000 |
| 1 | NOAvsRESP | CD.EpithStem.LINC00176.RPS4Y1 | 20 | 0.011 | 26037.520 | 0.000 |
| 2 | NOAvsRESP | CD.T.MKI67.CCR7 17.5 | 17.5 | 0.042 | 1189.726 | 0.000 |

| Cell | | | | | | |
|---|---|---|---|---|---|---|
| type | avg_log2FC | id.null | id.altr | fdr_full | fdr_celltype | fdr_celltype_response |
| 2 | −6.565 | NOA | PR | 0.721 | 0.333 | 0.158 |
| 6 | −6.104 | NOA | PR | 0.721 | 0.506 | 0.195 |
| 1 | −5.675 | NOA | RESP | 0.721 | 0.632 | 0.292 |
| 8 | −4.594 | NOA | RESP | 0.721 | 0.684 | 0.751 |
| 1 | −4.452 | NOA | RESP | 0.721 | 0.632 | 0.292 |
| 1 | −4.382 | NOA | PR | 0.816 | 0.632 | 0.363 |
| 8 | −4.161 | NOA | PR | 0.816 | 0.684 | 0.499 |
| 8 | −3.553 | NOA | PR | 0.816 | 0.684 | 0.499 |
| 6 | −3.489 | NOA | PR | 0.721 | 0.506 | 0.195 |
| 2 | −2.929 | NOA | PR | 0.721 | 0.333 | 0.158 |
| 6 | −2.812 | NOA | PR | 0.721 | 0.506 | 0.195 |
| 1 | −2.812 | NOA | RESP | 0.721 | 0.632 | 0.292 |
| 2 | −2.600 | NOA | PR | 0.816 | 0.452 | 0.201 |
| 8 | −1.990 | RESP | PR | 0.721 | 0.684 | 0.554 |
| 2 | −1.582 | RESP | PR | 0.721 | 0.333 | 0.821 |
| 6 | −1.289 | NOA | RESP | 0.721 | 0.506 | 0.857 |
| 9 | 1.260 | RESP | PR | 0.721 | 0.562 | 0.286 |
| 8 | 2.575 | RESP | PR | 0.816 | 0.684 | 0.554 |
| 8 | 2.575 | RESP | PR | 0.816 | 0.684 | 0.554 |
| 2 | 2.614 | NOA | PR | 0.816 | 0.452 | 0.201 |
| 2 | 2.721 | NOA | RESP | 0.721 | 0.333 | 0.517 |
| 2 | 2.833 | NOA | PR | 0.721 | 0.333 | 0.158 |
| 6 | 3.007 | NOA | PR | 0.721 | 0.506 | 0.195 |
| 1 | 3.298 | RESP | PR | 0.816 | 0.632 | 0.930 |
| 2 | 3.633 | NOA | PR | 0.721 | 0.333 | 0.158 |
| 2 | 3.957 | NOA | PR | 0.721 | 0.333 | 0.158 |
| 6 | 4.950 | NOA | PR | 0.721 | 0.506 | 0.195 |
| 2 | 5.041 | NOA | PR | 0.721 | 0.333 | 0.158 |
| 3 | 5.285 | RESP | PR | 0.816 | 0.987 | 0.907 |
| 2 | 5.537 | NOA | PR | 0.721 | 0.333 | 0.158 |
| 2 | 5.599 | NOA | RESP | 0.721 | 0.333 | 0.517 |
| 9 | Inf | NOA | PR | 0.816 | 0.562 | 0.467 |
| 1 | Inf | NOA | PR | 0.816 | 0.632 | 0.363 |
| 6 | Inf | NOA | PR | 0.816 | 0.719 | 0.368 |
| 1 | #NAME? | NOA | PR | 0.816 | 0.632 | 0.363 |
| 1 | #NAME? | NOA | PR | 0.816 | 0.632 | 0.363 |
| 1 | #NAME? | NOA | PR | 0.816 | 0.632 | 0.363 |
| 1 | #NAME? | NOA | PR | 0.816 | 0.632 | 0.363 |
| 8 | #NAME? | NOA | PR | 0.721 | 0.684 | 0.443 |
| 1 | #NAME? | NOA | PR | 0.816 | 0.632 | 0.363 |
| 3 | #NAME? | NOA | RESP | 0.816 | 0.987 | 0.765 |
| 1 | #NAME? | NOA | RESP | 0.816 | 0.632 | 0.451 |
| 1 | #NAME? | NOA | RESP | 0.816 | 0.632 | 0.451 |
| 1 | #NAME? | NOA | RESP | 0.721 | 0.632 | 0.292 |
| 2 | #NAME? | NOA | RESP | 0.816 | 0.543 | 0.517 |

TABLE 13
PC2 top positive and negative cell clusters

| | | | |
|---|---|---|---|
| PC2 most positive | | Mono.FCN1.LYST | −0.1076114 |
| T.MT.CO2.MT.CO1 | 0.10483214 | NK.MKI67.GZMA | −0.11073 |
| EC.GNAT3.TRPM5 | 0.09611848 | Mono.CXCL10.TNF | −0.1125706 |
| EC.RBP2.CYP3A4 | 0.09314928 | T.CARD16.GB2 | −0.1192552 |
| CDC2.CLEC10A.FCGR2B | 0.09072926 | Mono.FCN1.S100A4 | −0.1217137 |
| Myeloid. MKI67.IGKC | 0.08479524 | Mono. Mac.CXCL10.CXCL11 | −0.1229109 |
| Goblet.FCGBP.HES6 | 0.08392152 | NK.GNLY.GZMB | −0.1241935 |
| EC.ADH1C.EDN1 | 0.08372226 | EC.OLFM4.MT.ND2 | −0.1285151 |
| Mcell.CSRP2.SPIB | 0.080534 | NK.GNLY.IFNG | −0.1289419 |
| EC.FABP6.PLCG2 | 0.08019618 | T.MKI67.IL22 | −0.1326235 |
| EC.MTRNR2L1.MT.ND3 | 0.07622611 | NK.GNLY.FCER1G | −0.133085 |
| T.MT.CO2.CCR7 | 0.0748362 | Mac.DC.CXCL10.CLEC4E | −0.1336162 |
| EC.ADH1C.RPS4Y1 | 0.07479161 | T.MKI67.IFNG | −0.1365221 |
| T.IFI44L.PTGER4 | 0.07375801 | T.MKI67.FOXP3 | −0.1381972 |
| cDC2.CD1C.NDRG2 | 0.07302601 | Mono.Mac.CXCL10.FCN1 | −0.1390505 |
| Goblet.FCGBP.ITLN1 | 0.07243466 | T.GNLY.CSF2 | −0.1398405 |
| EC.FABP1.ADIRF | 0.07205058 | EC.NUPR1.LCN2 | −0.1401013 |
| EC.GSTA2.TMPRSS15 | 0.07109092 | Mac.CXCL3.APOC1 | −0.140698 |
| cDC2.CLEC10A.CD1E | 0.06972422 | pDC.IRF7.IL3RA | −0.150641 |
| Mac.CXCL8. HES1 | 0.06909605 | Mono.CXCL3.FCN1 | −0.1506537 |
| cDC2.CD1C.CD207 | 0.06837918 | Mac.C1QB.CD14 | −0.1537369 |
| Mono.cDC2.CLEC10A.SDS | 0.06837918 | Goblet.RETNLB.ITLN1 | −0.1547851 |
| T.LAG3.BATF | 0.06800467 | T.CCL20.RORA | −0.1605739 |
| Secretory.GSTA1.REG1B | 0.06632543 | T.MAF.CTLA4 | −0.161625 |
| EC.ADH1C.GSTA1 | 0.06566983 | cDC2.CD1C.AREG | −0.1668905 |
| EpithStem.LINC00176.RPS4Y1 | 0.06439734 | PC2 most negative | |

TABLE 14
92 markers derived from PC2 used for bulk RNA seq extension

| | |
|---|---|
| 1 | CD86 |
| 2 | LCN2 |
| 3 | REG3A |
| 4 | PRSS2 |
| 5 | PLA2G2A |
| 6 | FXYD3 |
| 7 | FCGR1A |
| 8 | FCN1 |
| 9 | CD14 |
| 10 | MS4A4A |
| 11 | GPNMB |
| 12 | CD163 |
| 13 | APOC1 |
| 14 | CCL4L2 |
| 15 | CXCL3 |
| 16 | TNF |
| 17 | CXCL9 |
| 18 | CXCL10 |
| 19 | CLEC4E |
| 20 | PLA2G7 |
| 21 | FAM26F |
| 22 | C1QB |
| 23 | RP11-1143G9.4 |
| 24 | FGL2 |
| 25 | IL1RN |
| 26 | S100A8 |
| 27 | TNFAIP6 |
| 28 | S100A9 |
| 29 | EREG |
| 30 | CCL3 |
| 31 | PLAUR |
| 32 | LINC01272 |
| 33 | FCGR3A |
| 34 | APOBEC3A |
| 35 | C5AR1 |
| 36 | SERPINA1 |
| 37 | OLR1 |
| 38 | CXCL11 |
| 39 | GBP1 |
| 40 | GNLY |
| 41 | KLRF1 |
| 42 | GZMB |
| 43 | KLRC1 |
| 44 | TRDC |
| 45 | KLRD1 |
| 46 | GZMA |
| 47 | GZMH |
| 48 | NKG7 |
| 49 | IL2RB |
| 50 | FGFBP2 |
| 51 | MYOM2 |
| 52 | PRF1 |
| 53 | CX3CR1 |
| 54 | CTSW |
| 55 | XCL1 |
| 56 | IFNG |
| 57 | XCL2 |
| 58 | ASPM |
| 59 | MKI67 |
| 60 | TOP2A |
| 61 | CENPF |
| 62 | TYMS |
| 63 | LILRA4 |
| 64 | PTCRA |
| 65 | SCT |
| 66 | IL3RA |
| 67 | SMPD3 |
| 68 | CLIC3 |
| 69 | PTPRS |
| 70 | MYBL2 |
| 71 | FOXP3 |
| 72 | RTKN2 |
| 73 | TIGIT |
| 74 | CXCR6 |
| 75 | IL32 |
| 76 | MIR4435-2HG |
| 77 | CTLA4 |
| 78 | AC092580.4 |
| 79 | CHN1 |
| 80 | RORA |
| 81 | S100A4 |
| 82 | IL26 |
| 83 | CSF2 |
| 84 | PYHIN1 |
| 85 | MAF |
| 86 | ICOS |
| 87 | BIRC5 |
| 88 | NUSAP1 |
| 89 | RRM2 |
| 90 | TK1 |
| 91 | ADA |
| 92 | DLGAP5 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating a subject suffering from inflammatory bowel disease (IBD) comprising:
determining whether the subject belongs to a risk group selected from: (i) well controlled without anti-TNF-blockade (NOA), (ii) anti-TNF-blockade full responder (FR), and (iii) anti-TNF-blockade partial responder (PR) by:
detecting in a sample obtained from the subject at diagnosis or before treatment the frequency of one or more T cell/Natural Killer/Innate lymphoid cell (T/NK/ILC), myeloid and/or epithelial cell subsets selected from Table 1,
determining the risk group of the subject by comparing the frequency of the detected cell subsets to a control frequency for the subsets along a trajectory of disease severity from NOA to FR to PR; and
if the subject is in the NOA group, then treating the subject with a treatment that does not comprise anti-TNF-blockade;
if the subject is in the FR group, then treating the subject with a treatment comprising anti-TNF-blockade;
if the subject is in the PR group, then treating the subject with a treatment comprising anti-TNF-blockade and/or an additional treatment.

2. The method of claim 1, wherein the cell subsets are selected from the group consisting of: CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, CD.Goblet.TFF1.TPSG1, CD.T.LAG3.BATF, CD.T.IFI44L.PTGER4, CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15,
wherein the frequency of the CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, and CD.Goblet.TFF1.TPSG1 subsets is increased in PR subjects as compared to NOA subjects, and
wherein the frequency of the CD.T.LAG3.BATF, CD.T.IFI44L.PTGER4, and CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15 subsets is decreased in PR subjects as compared to NOA subjects.

3. The method of claim 1, wherein the cell subsets are selected from the group consisting of: CD.NK.MKI67.GZMA, CD.T.MKI67.IL22, CD.Fibro.CCL19.IRF7 and CD.EC.SLC28A2.GSTA2,
wherein the frequency of the CD.NK.MKI67.GZMA and CD.T.MKI67.IL22 subsets is increased in FR and PR subjects as compared to NOA subjects, and
wherein the frequency of the CD.Fibro.CCL19.IRF7 and CD.EC.SLC28A2.GSTA2 subsets is decreased in FR and PR subjects as compared to NOA subjects.

4. The method of claim 1, wherein the cell subsets are selected from the group consisting of: cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, Mac.DC.CXCL10.CLEC4E, NK.GNLY.FCER1G, T.MKI67.IL22, NK.GNLY.IFNG, EC.OLFM4.MT.ND2, NK.GNLY.GZMB, Mono.Mac.CXCL10.CXCL11, Mono.FCN1.S100A4, T.CARD16.GB2, Mono.CXCL10.TNF, and NK.MKI67.GZMA,
wherein the frequency of at least one subset from each of the T/NK/ILC, myeloid and epithelial cell states subsets is increased in PR subjects as compared to FR and NOA subjects.

5. The method of claim 1, wherein the cell subsets are selected from the group consisting of: CD.EpithStem.LINC00176.RPS4Y1, CD.MCell.CSRP2.SPIB, CD.EC.FABP6.PLCG2, and CD.EC.FABP1.ADIRF,
wherein the frequency of the CD.EpithStem.LINC00176.RPS4Y1, CD.MCell.CSRP2.SPIB, CD.EC.FABP6.PLCG2, and CD.EC.FABP1.ADIRF subsets is decreased in FR subjects as compared to NOA subjects.

6. The method of claim 1, wherein the cell subset is the CD.B/DZ.HIST1H1B.MKI67 subset, wherein the frequency of the CD.B/DZ.HIST1H1B.MKI67 subset is increased in PR subjects as compared to FR subjects.

7. A method of treating a subject suffering from inflammatory bowel disease (IBD) comprising:
detecting in a sample obtained from the subject at diagnosis or before treatment the expression of one or more genes selected from Table 2;
determining whether the subject is in the FR or PR risk group by comparing to a control level in FR and/or PR subjects; and
if the subject is in the FR group, then treating the subject with a treatment comprising anti-TNF-blockade;
if the subject is in the PR group, then treating the subject with a treatment comprising anti-TNF-blockade and/or an additional treatment.

8. The method of claim 7, wherein the one or more genes are detected in one or more cell subsets selected from the group consisting of CD.NK.CCL3.CD160, CD.Fibro.TFPI2.CCL13, CD.Paneth.DEFA6.ITLN2 and CD.Mac.APOE.PTGDS, wherein the one or more cell subsets are detected according to one or more genes in Table 1.

9. The method of claim 7, wherein the one or more genes are selected from the group consisting of IFITM1, APOA1, TPT1, FABP6, NACA, APOA4, MIF, HOPX, SPINK4, CMC1, TNFRSF11B, BRI3, COL1A2, NKG7, APOE, TFPI2, AREG, KLRC1, HTRA3, COL1A1, HIF1A, STAT1, SLC16A4, SERPINE2, CCL11, SAMHD1, TAX1BP1, TXN, GPR65, CEBPB, GSN, EMILIN1, CTNNB1, COL4A1, CLEC12A, PTGER4, BDKRB1, SKIL, and PFN1,
wherein APOA1, FABP6, NACA, APOA4, TPT1, SPINK4, MIF, IFITM1, and HOPX are increased in FR relative to PR, and
wherein TNFRSF11B, TFPI2, SERPINE2, GSN, COL1A1, HIF1A, COL1A2, CTNNB1, CCL11, EMILIN1, CEBPB, SLC16A4, HTRA3, CMC1, AREG, COL4A1, SKIL, KLRC1, PTGER4, BRI3, APOE, BDKRB1, TXN, GPR65, NKG7, SAMHD1, CLEC12A, STAT1, PFN1, and TAX1BP1 are increased in PR relative to FR.

10. A method of treating a subject suffering from inflammatory bowel disease (IBD) comprising:
detecting in a sample obtained from the subject at diagnosis or before treatment the expression of one or more genes selected from the group consisting of TNFAIP6, GZMB, S100A8, CSF2, CLEC4E, S100A9, IL1RN, FCGR1A, CLIC3, CD14, PLA2G7, FAM26F, IL3RA, NKG7, IL32, CCL3, OLR1, LILRA4, APOC1, and MYBL2; or Table 14; and
if the subject has decreased expression of the one or more genes compared to a control, then treating the subject with a treatment comprising anti-TNF-blockade;
if the subject has increased expression of the one or more genes compared to a control, then treating the subject with a treatment comprising anti-TNF-blockade and/or an additional treatment.

11. The method of claim 1, wherein the anti-TNF-blockade is a monoclonal antibody.

12. A method of stratifying subjects suffering from IBD into a risk group comprising:
detecting in a sample obtained from a subject at diagnosis or before treatment the frequency of one or more T cell/Natural Killer/Innate lymphoid cell (T/NK/ILC), myeloid and/or epithelial cell subsets selected from Table 1, and
determining if the subject is in a well-controlled without anti-TNF-blockade (NOA) risk group, an anti-TNF-blockade full responder (FR) risk group, or anti-TNF-blockade partial responder (PR) risk group by comparing the frequency of the detected cell subsets to a control frequency for the subsets along a trajectory of disease severity from NOA to FR to PR; or
detecting in a sample obtained from a subject at diagnosis or before treatment the expression of one or more genes selected from the group consisting of TNFAIP6, GZMB, S100A8, CSF2, CLEC4E, S100A9, IL1RN, FCGR1A, CLIC3, CD14, PLA2G7, FAM26F, IL3RA, NKG7, IL32, CCL3, OLR1, LILRA4, APOC1, and MYBL2: or Table 14, and
determining if the subject is in a well-controlled without anti-TNF-blockade (NOA) risk group, an anti-TNF-blockade full responder (FR) risk group, or anti-TNF-blockade partial responder (PR) risk group by comparing the expression of the one or more genes to a control expression for the subsets along a trajectory of disease severity from NOA to FR to PR.

13. The method of claim 12, wherein the cell subsets are selected from the group consisting of: CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, CD.Goblet.TFF1.TPSG1, CD.T.LAG3.BATF, CD.T.IFI44L.PTGER4, CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15,
wherein the frequency of the CD.T.MKI67.IFNG, CD.T.MKI67.FOXP3, CD.T.GNLY.CSF2, CD.NK.GNLY.FCER1G, CD.Mac.CXCL3.APOC1, CD.Mono/Mac.CXCL10.FCN1, CD.Mono.FCN1.S100A4, CD.Endth/Ven.LAMP3.LIPG, and CD.Goblet.TFF1.TPSG1 subsets is increased in PR subjects as compared to NOA subjects, and
wherein the frequency of the CD.T.LAG3.BATF, CD.T.IFI44L.PTGER4, and CD.T.IFI6.IRF7, CD.cDC2.CLEC10A.FCGR2B, CD.Fibro.IFI6.IFI44L, CD.Tuft.GNAT3.TRPM5, CD.EC.GSTA2.CES3, and CD.EC.GSTA2.TMPRSS15 subsets is decreased in PR subjects as compared to NOA subjects.

14. The method of claim 12, wherein the cell subsets are selected from the group consisting of: CD.NK.MKI67.GZMA, CD.T.MKI67.IL22, CD.Fibro.CCL19.IRF7 and CD.EC.SLC28A2.GSTA2,
wherein the frequency of the CD.NK.MKI67.GZMA and CD.T.MKI67.IL22 subsets is increased in FR and PR subjects as compared to NOA subjects, and
wherein the frequency of the CD.Fibro.CCL19.IRF7 and CD.EC.SLC28A2.GSTA2 subsets is decreased in FR and PR subjects as compared to NOA subjects.

15. The method of claim 12, wherein the cell subsets are selected from the group consisting of: cDC2.CD1C.AREG, T.MAF.CTLA4, T.CCL20.RORA, Goblet.RETNLB.ITLN1, Mac.C1QB.CD14, Mono.CXCL3.FCN1, pDC.IRF7.IL3RA, Mac.CXCL3.APOC1, EC.NUPR1.LCN2, T.GNLY.CSF2, Mono.Mac.CXCL10.FCN1, T.MKI67.FOXP3, T.MKI67.IFNG, Mac.DC.CXCL10.CLEC4E, NK.GNLY.FCER1G, T.MKI67.IL22, NK.GNLY.IFNG, EC.OLFM4.MT.ND2, NK.GNLY.GZMB, Mono.Mac.CXCL10.CXCL11, Mono.FCN1.S100A4, T.CARD16.GB2, Mono.CXCL10.TNF, and NK.MKI67.GZMA,
wherein the frequency of at least one subset from each of the T/NK/ILC, myeloid and epithelial cell states subsets is increased in PR subjects as compared to FR and NOA subjects.

16. The method of claim 12, wherein the cell subsets are selected from the group consisting of. CD.EpithStem.LINC00176.RPS4Y1, CD.MCell.CSRP2.SPIB, CD.EC.FABP6.PLCG2, and CD.EC.FABP1.ADIRF,
wherein the frequency of the CD.EpithStem.LINC00176.RPS4Y1, CD.MCell.CSRP2.SPIB, CD.EC.FABP6.PLCG2, and CD.EC.FABP1.ADIRF subsets is decreased in FR subjects as compared to NOA subjects.

17. The method of claim 12, wherein the cell subset is the CD.B/DZ.HIST1H1B.MKI67 subset, wherein the frequency of the CD.B/DZ.HIST1H1B.MKI67 subset is increased in PR subjects as compared to FR subjects.

18. (canceled)

19. The method of claim 1, wherein the IBD is Crohn's Disease (CD).

20. The method of claim 1, wherein the cell states or genes are detected by RNA-seq, immunohistochemistry (IHC), fluorescently bar-coded oligonucleotide probes, RNA FISH, FACS, or any combination thereof, optionally,
wherein the cell states are inferred from bulk RNA-seq; or
wherein the cell states are determined by single cell RNA-seq.

21-22. (canceled)

23. The method of claim 1, wherein the sample is obtained by biopsy; and/or
wherein the subject is younger than 35, 25, 20, or 18 years old; and/or
wherein when the frequency of a cell state increases, the frequency of a cell state in the parent cells for the control subject is less than 0, 5, 10, or 50 percent of the parent cell; and/or
wherein when the frequency of a cell state decreases, the frequency of a cell state in the parent cells for the control subject is greater than 0, 5, 10, or 50 percent of the parent cell.

24-26. (canceled)

27. The method of claim 1, wherein the CD.NK.MKI67.GZMA cell state is detected by detecting one or more genes selected from the group consisting of GNLY, CCL3, KLRD1, IL2RB and EOMES; and/or
wherein the CD.T.MKI67.IL22 cell state is detected by detecting one or more genes selected from the group consisting of IFNG, CCL20, IL22, IL26, CD40LG and ITGAE; and/or
wherein the CD.Fibro.CCL9.IRF7 cell state is detected by detecting one or more genes selected from the group consisting of CCL19, CCL11, CXCL1, CCL2, OAS1 and IRF7; and/or
wherein the CD.EC.SLC28A2.GSTA2 cell state is detected by detecting one or more genes selected from the group consisting of SLC28A2 and GSTA2; and/or
wherein the CD.T.MKI67.IFNG cell state is detected by detecting one or more genes selected from the group consisting of IFNG, GNLY, HOPX, ITGAE and IL26; and/or
wherein the CD.T.MKI67.FOXP3cell state is detected by detecting one or more genes selected from the group consisting of IL2RA, BATF, CTLA4, TNFRSF1B, CXCR3, and FOXP3; and/or
wherein the CD.T.GNLY.CSF2 cell state is detected by detecting one or more genes selected from the group consisting of GNLY, GZMB, GZMA, PRF1, IFNG, CXCR6, and CSF2; and/or
wherein the CD.NK.GNLY.FCER1G cell state is detected by detecting one or more genes selected from the group consisting of GNLY, GZMB, GZMA, PRF1, AREG, TYROBP, and KLRF1; and/or
wherein the CD.Mac.CXCL3.APOC1 cell state is detected by detecting one or more genes selected from the group consisting of CCL3, CCL4, CXCL3, CXCL2, CXCL1, CCL20, CCL8, TNF and IL1B; and/or
wherein the CD.Mono/Mac.CXCL10.FCN1 cell state is detected by detecting one or more genes selected from the group consisting of CXCL9, CXCL10, CXCL11, GBP1, GBP2, GBP4, GBP5, and Type II IFN-gamma; and/or
wherein the CD.Mono.FCN1.S100A4 cell state is detected by detecting one or more genes selected from the group consisting of S100A4, S100A6, and FCN1.

28-37. (canceled)
